SEQUENCE LISTING (310 sequences)

SEQ ID NO: 1 - protein, 379 aa, Homo sapiens
MSSAPEKQQPPHGGGGGGGGGGGAAMDPASSGPSKAKKTNAGIRRPEKPPYSYIALIVMAIQSSPTKRLTLSEIYQFLQSRFPFFRGSYQGWKNSVRHNLSLNECFIKLPKGLGRPGKGHYWTIDPASEFMFEEGSFRRRPRGFRRKCQALKPMYSMMNGLGFNHLPDTYGFQGSAGGLSCPPNSLALEGGLGMMNGHLPGNVDGMALPSHSVPHLPSNGGHSYMGGCGGAAAGEYPHHDSSVPASPLLPTGAGGVMEPHAVYSGSAAAWPPSASAALNSGASYIKQQPLSPCNPAANPLSGSLSTHSLEQPYLHQNSHNAPAELQGIPRYHSQSPSMCDRKEFVFSFNAMASSSMHSAGGGSYYHQQVTYQDIKPCVM

SEQ ID NO: 2 - DNA, 1137 bp, Homo sapiens
  source 1..1137: mol_type = genomic DNA
atgtcttcggcgcccgagaagcagcagccaccgcacggcggcggcggcggcggcggcgggggaggcggcgcggccatggaccccgcgtcgtccggcccgtccaaggccaagaagaccaacgccggcatccggcgcccggagaagccgccctattcctacatcgcgctcatcgtcatggccatccagagttcacccaccaagcgcctgacgctgagcgagatctaccagttcctgcagagccgcttccccttcttccggggctcctaccagggctggaagaactccgtgcgccacaacctctcgctcaacgagtgcttcatcaagctacccaagggccttgggcggcccggcaagggccactactggaccatcgacccggccagcgagttcatgttcgaggagggctcctttcggcggcggccgcgcggcttccgaaggaaatgccaggcgctcaagcccatgtacagcatgatgaacgggctcggcttcaaccacctcccggacacctacggcttccagggctcggccggcggcctctcgtgcccgcccaacagcctggcgctggagggcggcctgggcatgatgaacggccacttgccgggcaacgtggacggcatggccctgcccagccactcggtgccccacctgccttccaacggcggccactcgtacatgggcggctgcggcggcgcggcggccggcgagtacccgcaccacgacagctcggtgcccgcctccccgctgctgcccaccggcgccggtggggtcatggagccgcacgccgtctactcgggctcggcggcggcctggccgccctcggcgtccgcggcgctcaacagcggcgcctcttatatcaagcagcagcccctgtccccctgtaaccccgcggccaaccccctgtccggcagcctctccacgcactccctggagcagccgtatctgcaccagaacagccacaacgccccagccgagctgcaaggcatcccgcggtatcactcgcagtcgcccagcatgtgtgaccgaaaggagtttgtcttctctttcaacgccatggcgtcctcttccatgcactcggccggcgggggctcctactaccaccagcaggtcacctaccaagacatcaagccttgcgtgatg

SEQ ID NO: 3 - protein, 472 aa, Homo sapiens
MLGTVKMEGHETSDWNSYYADTQEAYSSVPVSNMNSGLGSMNSMNTYMTMNTMTTSGNMTPASFNMSYANPGLGAGLSPGAVAGMPGGSAGAMNSMTAAGVTAMGTALSPSGMGAMGAQQAASMNGLGPYAAAMNPCMSPMAYAPSNLGRSRAGGGGDAKTFKRSYPHAKPPYSYISLITMAIQQAPSKMLTLSEIYQWIMDLFPYYRQNQQRWQNSIRHSLSFNDCFVKVARSPDKPGKGSYWTLHPDSGNMFENGCYLRRQKRFKCEKQPGAGGGGGSGSGGSGAKGGPESRKDPSGASNPSADSPLHRGVHGKTGQLEGAPAPGPAASPQTLDHSGATATGGASELKTPASSTAPPISSGPGALASVPASHPAHGLAPHESQLHLKGDPHYSFNHPFSINNLMSSSEQQHKLDFKAYEQALQYSPYGSTLPASLPLGSASVTTRSPIEPSALEPAYYQGVYSRPVLNTS

SEQ ID NO: 4 - DNA, 1416 bp, Homo sapiens
  source 1..1416: mol_type = genomic DNA
atgttaggaactgtgaagatggaagggcatgaaaccagcgactggaacagctactacgcagacacgcaggaggcctactcctccgtcccggtcagcaacatgaactcaggcctgggctccatgaactccatgaacacctacatgaccatgaacaccatgactacgagcggcaacatgaccccggcgtccttcaacatgtcctatgccaacccgggcctaggggccggcctgagtcccggcgcagtagccggcatgccggggggctcggcgggcgccatgaacagcatgactgcggccggcgtgacggccatgggtacggcgctgagcccgagcggcatgggcgccatgggtgcgcagcaggcggcctccatgaatggcctgggcccctacgcggccgccatgaacccgtgcatgagccccatggcgtacgcgccgtccaacctgggccgcagccgcgcgggcggcggcggcgacgccaagacgttcaagcgcagctacccgcacgccaagccgccctactcgtacatctcgctcatcaccatggccatccagcaggcgcccagcaagatgctcacgctgagcgagatctaccagtggatcatggacctcttcccctattaccggcagaaccagcagcgctggcagaactccatccgccactcgctgtccttcaatgactgcttcgtcaaggtggcacgctccccggacaagccgggcaagggctcctactggacgctgcacccggactccggcaacatgttcgagaacggctgctacttgcgccgccagaagcgcttcaagtgcgagaagcagccgggggccggcggcgggggcgggagcggaagcgggggcagcggcgccaagggcggccctgagagccgcaaggacccctctggcgcctctaaccccagcgccgactcgcccctccatcggggtgtgcacgggaagaccggccagctagagggcgcgccggcccccgggcccgccgccagcccccagactctggaccacagtggggcgacggcgacagggggcgcctcggagttgaagactccagcctcctcaactgcgccccccataagctccgggcccggggcgctggcctctgtgcccgcctctcacccggcacacggcttggcaccccacgagtcccagctgcacctgaaaggggacccccactactccttcaaccacccgttctccatcaacaacctcatgtcctcctcggagcagcagcataagctggacttcaaggcatacgaacaggcactgcaatactcgccttacggctctacgttgcccgccagcctgcctctaggcagcgcctcggtgaccaccaggagccccatcgagccctcagccctggagccggcgtactaccaaggtgtgtattccagacccgtcctaaacacttcc

SEQ ID NO: 5 - protein, 457 aa, Homo sapiens
MLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 6 - DNA, 1371 bp, Homo sapiens
  source 1..1371: mol_type = genomic DNA
atgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggctggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggggcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccagccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctct

SEQ ID NO: 7 - protein, 350 aa, Homo sapiens
MLGSVKMEAHDLAEWSYYPEAGEVYSPVTPVPTMAPLNSYMTLNPLSSPYPPGGLPASPLPSGPLAPPAPAAPLGPTFPGLGVSGGSSSSGYGAPGPGLVHGKEMPKGYRRPLAHAKPPYSYISLITMAIQQAPGKMLTLSEIYQWIMDLFPYYRENQQRWQNSIRHSLSFNDCFVKVARSPDKPGKGSYWALHPSSGNMFENGCYLRRQKRFKLEEKVKKGGSGAATTTRNGTGSAASTTTPAATVTSPPQPPPPAPEPEAQGGEDVGALDCGSPASSTPYFTGLELPGELKLDAPYNFNHPFSINNLMSEQTPAPPKLDVGFGGYGAEGGEPGVYYQGLYSRSLLNAS

SEQ ID NO: 8 - DNA, 1050 bp, Homo sapiens
  source 1..1050: mol_type = genomic DNA
atgctgggctcagtgaagatggaggcccatgacctggccgagtggagctactacccggaggcgggcgaggtctactcgccggtgaccccagtgcccaccatggcccccctcaactcctacatgaccctgaatcctctaagctctccctatccccctggggggctccctgcctccccactgccctcaggacccctggcacccccagcacctgcagcccccctggggcccactttcccaggcctgggtgtcagcggtggcagcagcagctccgggtacggggccccgggtcctgggctggtgcacgggaaggagatgccgaaggggtatcggcggcccctggcacacgccaagccaccgtattcctatatctcactcatcaccatggccatccagcaggcgccgggcaagatgctgaccttgagtgaaatctaccagtggatcatggacctcttcccttactaccgggagaatcagcagcgctggcagaactccattcgccactcgctgtctttcaacgactgcttcgtcaaggtggcgcgttccccagacaagcctggcaagggctcctactgggccctacaccccagctcagggaacatgtttgagaatggctgctacctgcgccgccagaaacgcttcaagctggaggagaaggtgaaaaaagggggcagcggggctgccaccaccaccaggaacgggacagggtctgctgcctcgaccaccacccccgcggccacagtcacctccccgccccagcccccgcctccagcccctgagcctgaggcccagggcggggaagatgtgggggctctggactgtggctcacccgcttcctccacaccctatttcactggcctggagctcccaggggagctgaagctggacgcgccctacaacttcaaccaccctttctccatcaacaacctaatgtcagaacagacaccagcacctcccaaactggacgtggggtttgggggctacggggctgaaggtggggagcctggagtctactaccagggcctctattcccgctctttgcttaatgcatcc

SEQ ID NO: 9 - protein, 325 aa, Homo sapiens
MPRPGRNTYSDQKPPYSYISLTAMAIQSSPEKMLPLSEIYKFIMDRFPYYRENTQRWQNSLRHNLSFNDCFIKIPRRPDQPGKGSFWALHPSCGDMFENGSFLRRRKRFKVLKSDHLAPSKPADAAQYLQQQAKLRLSALAASGTHLPQMPAAAYNLGGVAQPSGFKHPFAIENIIAREYKMPGGLAFSAMQPVPAAYPLPNQLTTMGSSLGTGWPHVYGSAGMIDSATPISMASGDYSAYGVPLKPLCHAAGQTLPAIPVPIKPTPAAVPALPALPAPIPTLLSNSPPSLSPTSSQTATSQSSPATPSETLTSPASALHSVAVH

SEQ ID NO: 10 - DNA, 975 bp, Homo sapiens
  source 1..975: mol_type = genomic DNA
atgcctcggcccggccgcaacacgtacagcgaccagaagccgccctactcgtacatctcgctgaccgctatggccatccagagctctcccgagaagatgctgccgctgagcgagatctacaagttcatcatggaccgcttcccctactacagggagaacacgcagcgctggcagaacagtctgcgccacaacctctccttcaacgactgcttcatcaagatcccgcggcggccggaccagccaggcaagggcagcttctgggcgctgcacccaagctgcggggacatgttcgagaacggcagcttcctgcggcgccgcaagcgcttcaaggtgcttaagtccgaccacctggcgcccagcaagccagccgacgcggcgcagtacctgcagcagcaggccaagctgcggctcagcgcgctggcggcctcgggcacgcacctgccacagatgcccgccgccgcctacaacttgggcggcgtggcgcagccctcgggcttcaagcaccccttcgccatcgagaacatcatcgcgcgggaatacaagatgcctggggggctggccttctccgccatgcagccggtgcccgctgcctacccgctccccaaccagttgactaccatgggcagctcgctgggcaccggctggccacacgtgtatggctccgccggcatgatcgactcggccacccccatctccatggcgagtggcgactacagcgcctacggcgtgccgttgaagccgctgtgccacgcggcgggccaaacgctgcccgccatccccgtgcccattaagcccacgccggccgccgtgcccgcgctgcctgcgctgccagcgcccatccccaccttgctctcgaactcgccgccctcgctcagccccacgtcctcgcaaacagccaccagccaaagcagccccgccacccccagcgaaacgctcaccagcccggcctccgccttgcactcggtggcggtgcac

SEQ ID NO: 11 - protein, 432 aa, Homo sapiens
MPRPGKSSYSDQKPPYSYISLTAMAIQHSAEKMLPLSDIYKFIMERFPYYREHTQRWQNSLRHNLSFNDCFIKIPRRPDQPGKGSFWALHPDCGDMFENGSFLRRRKRFKVLRADHTHLHAGSTKSAPGAGPGGHLHPHHHHHPHHHHHHHAAAHHHHHHHPPQPPPPPPPPPPHMVHYFHQQPPTAPQPPPHLPSQPPQQPPQQSQPQQPSHPGKMQEAAAVAAAAAAAAAAAVGSVGRLSQFPPYGLGSAAAAAAAAAASTSGFKHPFAIENIIGRDYKGVLQAGGLPLASVMHHLGYPVPGQLGNVVSSVWPHVGVMDSVAAAAAAAAAAGVPVGPEYGAFGVPVKSLCHSASQSLPAMPVPIKPTPALPPVSALQPGLTVPAASQQPPAPSTVCSAAAASPVASLLEPTAPTSAESKGGSLHSVLVHS

SEQ ID NO: 12 - DNA, 1295 bp, Homo sapiens
  source 1..1295: mol_type = genomic DNA
atgcctcggcccggcaagtcatcttattctgatcaaaagccaccctactcatatattagcctcacagcgatggctatacagcattcagctgagaagatgttgcctctctccgacatctacaaatcatcatggagcggttcccctactaccgcgaacacacccagcggtggcagaactcacttagacacaacctgagcttcaatgattgttttattaagattcccaggaggccggaccagccaggcaagggttcattctgggcactccaccccgattgcggagacatgtttgaaaacgggagctttctccgacgacggaagagatttaaggtcctgagagccgatcatacccatctccacgccgggtccactaaatctgcaccgggggccggcccaggcgggcatctccatccccaccaccaccatcacccccatcaccatcatcatcaccacgccgctgcacaccaccaccatcaccaccaccccccacaaccaccccctcccccgccacccccgccaccccacatggtccactactttcaccaacagccccccaccgccccgcagcccccgccccacctgccatcacagcccccccagcagcccccacagcaaagccagccccagcaacctagccatcctggtaaaatgcaggaggctgcggcggtggctgcggctgcagctgccgctgctgctgcggctgttgggtctgtgggcagactgagccagttccctccctacggcttgggttccgccgccgcggcggccgccgccgctgcagccagcacttccggctttaagcatccatttgctattgagaacatcattggccgcgactataaaggcgtcctccaagccggaggactcccactcgcgagtgtgatgcatcacttgggctatccagtgccaggccagctgggtaacgtcgtgtcctccgtctggccccacgtgggggtaatggacagtgtggcagcagccgctgccgctgcagctgccgctggcgttccagtaggtcccgaatatggagcattcggcgtgcccgtgaagtccctgtgccactctgcaagccagagcctgccagccatgccggtgcccatcaagccaacaccagccctcccaccagtgtctgccttgcagccaggactcacggtgcccgccgcatctcagcagcctccagcaccctcaacggtgtgcagcgccgcagccgctagccccgtggccagcctcctggaacccactgcacccacatcagctgagtcaaaaggtggaagccttcattccgtgttggtgcactca

SEQ ID NO: 13 - protein, 553 aa, Homo sapiens
MQARYSVSSPNSLGVVPYLGGEQSYYRAAAAAAGGGYTAMPAPMSVYSHPAHAEQYPGGMARAYGPYTPQPQPKDMVKPPYSYIALITMAIQNAPDKKITLNGIYQFIMDRFPFYRDNKQGWQNSIRHNLSLNECFVKVPRDDKKPGKGSYWTLDPDSYNMFENGSFLRRRRRFKKKDAVKDKEEKDRLHLKEPPPPGRQPPPAPPEQADGNAPGPQPPPVRIQDIKTENGTCPSPPQPLSPAAALGSGSAAAVPKIESPDSSSSSLSSGSSPPGSLPSARPLSLDGADSAPPPPAPSAPPPHHSQGFSVDNIMTSLRGSPQSAAAELSSGLLASAAASSRAGIAPPLALGAYSPGQSSLYSSPCSQTSSAGSSGGGGGGAGAAGGAGGAGTYHCNLQAMSLYAAGERGGHLQGAPGGAGGSAVDDPLPDYSLPPVTSSSSSSLSHGGGGGGGGGGQEAGHHPAAHQGRLTSWYLNQAGGDLGHLASAAAAAAAAGYPGQQQNFHSVREMFESQRIGLNNSPVNGNSSCQMAFPSSQSLYRTSGAFVYDCSKF

SEQ ID NO: 14 - DNA, 1659 bp, Homo sapiens
  source 1..1659: mol_type = genomic DNA
atgcaggcgcgctactccgtgtccagccccaactccctgggagtggtgccctacctcggcggcgagcagagctactaccgcgcggcggccgcggcggccgggggcggctacaccgccatgccggcccccatgagcgtgtactcgcaccctgcgcacgccgagcagtacccgggcggcatggcccgcgcctacgggccctacacgccgcagccgcagcccaaggacatggtgaagccgccctatagctacatcgcgctcatcaccatggccatccagaacgccccggacaagaagatcaccctgaacggcatctaccagttcatcatggaccgcttccccttctaccgggacaacaagcagggctggcagaacagcatccgccacaacctctcgctcaacgagtgcttcgtcaaggtgccgcgcgacgacaagaagccgggcaagggcagctactggacgctggacccggactcctacaacatgttcgagaacggcagcttcctgcggcggcggcggcgcttcaagaagaaggacgcggtgaaggacaaggaggagaaggacaggctgcacctcaaggagccgcccccgcccggccgccagcccccgcccgcgccgccggagcaggccgacggcaacgcgcccggtccgcagccgccgcccgtgcgcatccaggacatcaagaccgagaacggtacgtgcccctcgccgccccagcccctgtccccggccgccgccctgggcagcggcagcgccgccgcggtgcccaagatcgagagccccgacagcagcagcagcagcctgtccagcgggagcagccccccgggcagcctgccgtcggcgcggccgctcagcctggacggtgcggattccgcgccgccgccgcccgcgccctccgccccgccgccgcaccatagccagggcttcagcgtggacaacatcatgacgtcgctgcgggggtcgccgcagagcgcggccgcggagctcagctccggccttctggcctcggcggccgcgtcctcgcgcgcggggatcgcacccccgctggcgctcggcgcctactcgcccggccagagctccctctacagctccccctgcagccagacctccagcgcgggcagctcgggcggcggcggcggcggcgcgggggccgcggggggcgcgggcggcgccgggacctaccactgcaacctgcaagccatgagcctgtacgcggccggcgagcgcgggggccacttgcagggcgcgcccgggggcgcgggcggctcggccgtggacgaccccctgcccgactactctctgcctccggtcaccagcagcagctcgtcgtccctgagtcacggcggcggcggcggcggcggcgggggaggccaggaggccggccaccaccctgcggcccaccaaggccgcctcacctcgtggtacctgaaccaggcgggcggagacctgggccacttggcgagcgcggcggcggcggcggcggccgcaggctacccgggccagcagcagaacttccactcggtgcgggagatgttcgagtcacagaggatcggcttgaacaactctccagtgaacgggaatagtagctgtcaaatggccttcccttccagccagtctctgtaccgcacgtccggagctttcgtctacgactgtagcaagttt

SEQ ID NO: 15 - protein, 501 aa, Homo sapiens
MQARYSVSDPNALGVVPYLSEQNYYRAAGSYGGMASPMGVYSGHPEQYSAGMGRSYAPYHHHQPAAPKDLVKPPYSYIALITMAIQNAPEKKITLNGIYQFIMDRFPFYRENKQGWQNSIRHNLSLNECFVKVPRDDKKPGKGSYWTLDPDSYNMFENGSFLRRRRRFKKKDVSKEKEERAHLKEPPPAASKGAPATPHLADAPKEAEKKVVIKSEAASPALPVITKVETLSPESALQGSPRSAASTPAGSPDGSLPEHHAAAPNGLPGFSVENIMTLRTSPPGGELSPGAGRAGLVVPPLALPYAAAPPAAYGQPCAQGLEAGAAGGYQCSMRAMSLYTGAERPAHMCVPPALDEALSDHPSGPTSPLSALNLAAGQEGALAATGHHHQHHGHHHPQAPPPPPAPQPQPTPQPGAAAAQAASWYLNHSGDLNHLPGHTFAAQQQTFPNVREMFNSHRLGIENSTLGESQVSGNASCQLPYRSTPPLYRHAAPYSYDCTKY

SEQ ID NO: 16 - DNA, 1503 bp, Homo sapiens
  source 1..1503: mol_type = genomic DNA
atgcaggcgcgctactccgtgtccgaccccaacgccctgggagtggtgccctacctgagcgagcagaattactaccgggctgcgggcagctacggcggcatggccagccccatgggcgtctattccggccacccggagcagtacagcgcggggatgggccgctcctacgcgccctaccaccaccaccagcccgcggcgcctaaggacctggtgaagccgccctacagctacatcgcgctcatcaccatggccatccagaacgcgcccgagaagaagatcaccttgaacggcatctaccagttcatcatggaccgcttccccttctaccgggagaacaagcagggctggcagaacagcatccgccacaacctctcgctcaacgagtgcttcgtcaaggtgccccgcgacgacaagaagcccggcaagggcagttactggaccctggacccggactcctacaacatgttcgagaacggcagcttcctgcggcgccggcggcgcttcaaaaagaaggacgtgtccaaggagaaggaggagcgggcccacctcaaggagccgcccccggcggcgtccaagggcgccccggccaccccccacctagcggacgcccccaaggaggccgagaagaaggtggtgatcaagagcgaggcggcgtccccggcgctgccggtcatcaccaaggtggagacgctgagccccgagagcgcgctgcagggcagcccgcgcagcgcggcctccacgcccgccggctcccccgacggctcgctgccggagcaccacgccgcggcgcccaacgggctgcctggcttcagcgtggagaacatcatgaccctgcgaacgtcgccgccgggcggagagctgagcccgggggccggacgcgcgggcctggtggtgccgccgctggcgctgccctacgccgccgcgccgcccgccgcctacggccagccgtgcgctcagggcctggaggccggggccgccgggggctaccagtgcagcatgcgagcgatgagcctgtacaccggggccgagcggccggcgcacatgtgcgtcccgcccgccctggacgaggccctctcggaccacccgagcggccccacgtcgcccctgagcgctctcaacctcgccgccggccaggagggcgcgctcgccgccacgggccaccaccaccagcaccacggccaccaccacccgcaggcgccgccgcccccgccggctccccagccccagccgacgccgcagcccggggccgccgcggcgcaggcggcctcctggtatctcaaccacagcggggacctgaaccacctccccggccacacgttcgcggcccagcagcaaactttccccaacgtgcgggagatgttcaactcccaccggctggggattgagaactcgaccctcggggagtcccaggtgagtggcaatgccagctgccagctgccctacagatccacgccgcctctctatcgccacgcagccccctactcctacgactgcacgaaatac

SEQ ID NO: 17 - protein, 465 aa, Homo sapiens
MTLSTEMSDASGLAEETDIDVVGEGEDEEDEEEEDDDEGGGGGPRLAVPAQRRRRRRSYAGEDELEDLEEEEDDDDILLAPPAGGSPAPPGPAPAAGAGAGGGGGGGGAGGGGSAGSGAKNPLVKPPYSYIALITMAILQSPKKRLTLSEICEFISGRFPYYREKFPAWQNSIRHNLSLNDCFVKIPREPGNPGKGNYWTLDPESADMFDNGSFLRRRKRFKRQPLLPPNAAAAESLLLRGAGAAGGAGDPAAAAALFPPAPPPPPHAYGYGPYGCGYGLQLPPYAPPSALFAAAAAAAAAAAFHPHSPPPPPPPHGAAAELARTAFGYRPHPLGAALPGPLPASAAKAGGPGASALARSPFSIESIIGGSLGPAAAAAAAAQAAAAAQASPSPSPVAAPPAPGSSGGGCAAQAAVGPAAALTRSLVAAAAAAASSVSSSAALGTLHQGTALSSVENFTARISNC

SEQ ID NO: 18 - DNA, 1395 bp, Homo sapiens
  source 1..1395: mol_type = genomic DNA
atgaccctgagcactgagatgtccgatgcctctggcctcgccgaggaaacagacatcgacgtggtgggggagggcgaggacgaagaagacgaggaagaggaggacgacgacgagggcggcggtggcgggccccggctggctgtccccgcgcagcggcggcggcggcggcgctcgtacgccggggaggacgagctggaggatctggaggaggaggaggacgacgatgacatcctgctggccccgcctgctgggggctccccggcgcccccgggcccggccccggcggcgggggcaggagccggtgggggcggcggcggcggcggcgcgggcggcggcgggagcgcgggtagcggcgccaagaacccgctggtgaagccgccctactcgtatatcgcgctcatcactatggccatcctgcagagccccaagaagcggctgacgctgagcgagatctgtgagttcatcagcggccgcttcccctactaccgggagaagttccccgcctggcagaacagcatccgccacaacctctcgctcaacgactgcttcgtcaagatcccccgcgagcccggcaacccgggcaagggcaactactggacgctggacccggagtccgccgacatgttcgacaacggcagcttcctgcgccggaggaagcgcttcaagcggcagccgctgctcccacccaacgccgcggccgccgagtctctgctgctgcgcggcgcgggagccgcagggggcgcgggcgacccggcagccgccgccgcgctcttcccgcccgcgcccccgccgcccccgcatgcctacggctacggcccctacggctgcggctacggcctgcagctgccgccttacgcgccgccctcggccctcttcgccgccgcagcggccgccgccgccgccgccgccttccacccgcactcgcccccgccgcccccgccaccgcacggcgcggccgccgagctggcccggaccgccttcggctaccggccgcacccgctcggcgccgccctacccggccccctgccggcctccgcggccaaggcgggcggcccgggcgcctcagcgctggcgcgctcgcccttctccatcgagagcatcatcgggggcagcttgggcccggccgccgctgccgccgccgccgcgcaggccgccgccgccgctcaggcctcgccctcgccctcgccggtggcggcgccgccagctcccggatccagcggaggaggctgcgcggcgcaggcggccgtgggcccggcggccgcgctcacccgatccctcgtggccgccgcggccgccgccgcctcctcagtctcctcgtccgccgccttggggactctgcaccaagggactgccctgtccagtgtcgagaactttactgctaggatttccaattgt

SEQ ID NO: 19 - protein, 495 aa, Homo sapiens
MTLGSCCCEIMSSESSPAALSEADADIDVVGGGSGGGELPARSGPRAPRDVLPHGHEPPAEEAEADLAEDEEESGGCSDGEPRALASRGAAAAAGSPGPGAAAARGAAGPGPGPPSGGAATRSPLVKPPYSYIALITMAILQSPKKRLTLSEICEFISGRFPYYREKFPAWQNSIRHNLSLNDCFVKIPREPGNPGKGNYWTLDPESADMFDNGSFLRRRKRFKRQPLPPPHPHPHPHPELLLRGGAAAAGDPGAFLPGFAAYGAYGYGYGLALPAYGAPPPGPAPHPHPHPHAFAFAAAAAAAPCQLSVPPGRAAAPPPGPPTASVFAGAGSAPAPAPASGSGPGPGPAGLPAFLGAELGCAKAFYPASLSPPAAGTAAGLPTALLRQGLKTDAGGGAGGGGAGAGQRPSFSIDHIMGHGGGGAAPPGAGEGSPGPPFAAAAGPGGQAQVLAMLTAPALAPVAGHIRLSHPGDALLSSGSRFASKVAGLSGCHF

SEQ ID NO: 20 - DNA, 1485 bp, Homo sapiens
  source 1..1485: mol_type = genomic DNA
atgaccctgggcagctgctgctgcgagatcatgtcctccgagagctccccggccgcgctgtccgaggccgacgcagacatagacgtggtgggcggcggcagcggcgggggggagctcccagctcgctccgggccccgcgccccccgggacgtgctcccccacggccacgagcctcccgcggaggaagccgaggcagacttagccgaggacgaggaggagtctggtggctgctcggacggcgagccccgcgctctggcgtcccggggggcggcggccgcagcggggagcccggggccaggcgccgcggcggcccgcggcgcagcggggcccgggccgggaccgccgtcggggggcgcggcgacgcggagcccgctggtgaagccgccctactcgtacatcgcgctcatcaccatggccatcctgcagagccccaagaagcggctgacgttgagcgagatctgcgagttcatcagcggccgcttcccctactaccgggagaagttccccgcctggcagaacagcatccgccacaacctctctctcaacgactgcttcgtcaagatcccccgcgagccgggcaacccgggcaagggcaactactggacgctggacccggagtcggccgacatgttcgacaacggcagcttcctgcggcgtcgcaagcgcttcaagcggcagcccctgccgccgccgcacccacacccgcaccctcacccggagctgctgctgcgtggcggggccgcggcggcgggggatcccggcgctttcctgcccggcttcgctgcctacggcgcctacggctacggctacgggctggctctcccggcctacggcgcacccccgccggggccggccccgcatccgcacccgcacccgcacgccttcgctttcgccgcggcagccgccgccgctccttgccagctgtcggtacccccaggccgcgccgccgcgcctccacccggacctccgacggcctcggtgttcgcaggcgcgggatcggccccagctcctgcgcctgcctcaggctcgggcccgggcccgggccccgcaggcctgcccgccttcctgggcgcggagctgggctgcgccaaagccttctacccggcgtccctgagtcctcccgcagccggcaccgcggcgggtctgcccaccgcacttctgcgccagggcctcaagacggacgcgggcggtggtgcaggcggcgggggcgccggggcagggcagaggccttccttctctatagaccacatcatgggccacggtggcggcggggcagcacccccgggcgccggcgagggctctccgggaccgccattcgcggcagccgcgggtcctgggggccaagcccaggtcttggccatgctgactgctccggccctggctcccgttgctggccacattcgcctctcgcatcccggggacgcgctgctgtcctcagggtcccggtttgccagcaaagtcgccggccttagtggctgccacttc

SEQ ID NO: 21 - protein, 478 aa, Homo sapiens
MTLSGGGSASDMSGQTVLTAEDVDIDVVGEGDDGLEEKDSDAGCDSPAGPPELRLDEADEVPPAAPHHGQPQPPHQQPLTLPKEAAGAGAGPGGDVGAPEADGCKGGVGGEEGGASGGGPGAGSGSAGGLAPSKPKNSLVKPPYSYIALITMAILQSPQKKLTLSGICEFISNRFPYYREKFPAWQNSIRHNLSLNDCFVKIPREPGNPGKGNYWTLDPQSEDMFDNGSFLRRRKRFKRHQQEHLREQTALMMQSFGAYSLAAAAGAAGPYGRPYGLHPAAAAGAYSHPAAAAAAAAAAALQYPYALPPVAPVLPPAVPLLPSGELGRKAAAFGSQLGPGLQLQLNSLGAAAAAAGTAGAAGTTASLIKSEPSARPSFSIENIIGGGPAAPGGSAVGAGVAGGTGGSGGGSTAQSFLRPPGTVQSAALMATHQPLSLSRTTATIAPILSVPLSGQFLQPAASAAAAAAAAAQAKWPAQ

SEQ ID NO: 22 - DNA, 1434 bp, Homo sapiens
  source 1..1434: mol_type = genomic DNA
atgaccctctccggcggcggcagcgccagcgacatgtccggccagacggtgctgacggccgaggacgtggacatcgatgtggtgggcgagggcgacgacgggctggaagagaaggacagcgacgcaggttgcgatagccccgcggggccgccggagctgcgcctggacgaggcggacgaggtgcccccggcggcaccccatcacggacagcctcagccgccccaccagcagcccctgacattgcccaaggaggcggccggagccggggccggaccggggggcgacgtgggcgcgccggaggcggacggctgcaagggcggtgttggcggcgaggagggcggcgcgagcggcggcgggcctggcgcgggcagcggttcggcgggaggcctggccccgagcaagcccaagaacagcctagtgaagccgccttactcgtacatcgcgctcatcaccatggccatcctgcagagcccgcagaagaagctgaccctgagcggcatctgcgagttcatcagcaaccgcttcccctactacagggagaagttccccgcctggcagaacagcatccgccacaacctctcactcaacgactgcttcgtcaagatcccccgcgagccgggcaacccgggcaagggcaactactggaccctggacccgcagtccgaggacatgttcgacaacggcagcttcctgcggcgccggaaacgcttcaagcgccaccagcaggagcacctgcgcgagcagacggcgctcatgatgcagagcttcggcgcttacagcctggcggcggcggccggcgccgcgggaccctacggccgcccctacggcctgcaccctgcggcggcggccggtgcctattcgcacccggcagcggcggcggccgcggctgctgcggcggcgctccagtacccgtacgcgctgccgccggtggcaccggtgctgcctcccgctgtgccgctgctgccctcgggcgagctgggccgcaaagcggccgccttcggctcacagctcggcccgggcctgcagctgcagctcaatagcctgggcgccgccgcggccgctgcgggcacagcgggcgccgcgggcaccaccgcgtcgctcatcaagtccgagccaagcgcgcggccgtcgttcagcatcgagaacatcataggtgggggccccgcggctcctgggggctcggcggtgggcgctggggtcgccggcggcactgggggttcagggggcggcagcacggcgcagtcgtttctgcggccacccgggaccgtgcagtcggcagcgctcatggccacccaccaaccgctgtcgctgagccggacgactgccaccatcgcgcccattcttagcgtgccactctccggacagtttctgcagcccgcagcctcggccgccgccgctgctgcggccgccgctcaagccaaatggccggcgcaa

SEQ ID NO: 23 - protein, 439 aa, Homo sapiens
MNLPRAERLRSTPQRSLRDSDGEDGKIDVLGEEEDEDEEEAASQQFLEQSLQPGLQVARWGGVALPREHIEGGGGPSDPSEFGTEFRAPPRSAAASEDARQPAKPPSSYIALITMAILQSPHKRLTLSGICAFISDRFPYYRRKFPAWQNSIRHNLSLNDCFVKIPREPGRPGKGNYWSLDPASQDMFDNGSFLRRRKRFQRHQPTPGAHLPHPFPLPAAHAALHNPRPGPLLGAPAPPQPVPGAYPNTGPGRRPYALLHPHPPRYLLLSAPAYAGAPKKAEGADLATPAPFPCCSPHLVLSLGRRARVWRRHREADASLSALRVSCKGSGERVQGLRRVCPRPRGATAPCSSDRQACRTILQQQQRHQEEDCANGCAPTKGAVLGGHLSAASALLRYQAVAEGSGLTSLAAPLGGEGTSPVFLVSPTPSSLAESAGPS

SEQ ID NO: 24 - DNA, 1317 bp, Homo sapiens
  source 1..1317: mol_type = genomic DNA
atgaacttgccaagagctgagcgccttcgctccacaccgcagcgcagcctccgggactccgatggggaagacggtaaaatcgatgtcctgggagaggaggaagatgaagacgaggaggaggcggcgagccagcagttcctagagcagtcgctccagccggggctgcaggtggcccggtggggcggggttgcgcttccccgagagcacatcgagggcggcggcggcccgagcgacccctcagagtttggcaccgagttcagggcaccgccaaggtctgcggcggcctctgaagatgcccggcagccggcaaagcccccctcctcgtacatcgcgctcatcaccatggccatcctgcaaagcccgcacaagcgcctcacgctcagcggcatctgcgccttcattagtgaccgcttcccctactaccgccgcaagttccccgcctggcagaacagcatccgccacaacctctcgctgaacgactgcttcgtcaagatcccccgcgagccgggccgcccaggcaagggcaactactggagcctggaccccgcctcccaggacatgttcgacaatggcagctttctccggcgtaggaagcgtttccagcgccaccaaccgaccccgggagcccacctgccccaccccttccctctacctgctgcacacgccgccctgcacaacccccgcccaggccctctgcttggggcccctgccccgccgcagccagtcccgggggcctaccccaacaccggccccgggagacgcccttacgctctgctgcacccgcatcctcctcgctacctactgctctcggcccccgcctatgccggggcaccgaagaaagcagaaggcgcggacctggcgaccccggcacccttcccgtgctgcagccctcacttggtcctcagccttgggaggagggcaagggtctggcgtcgccaccgggaggcggatgcatctctttcagcattgagagtatcatgcaaggggtcaggggagcgggtacaggggctgcgcagagtttgtccccgaccgcgtggagctactgccccctgctccagcgaccgtcaagcctgtcggacaattttgcagcaacagcagcggcatcaggaggaggactgcgccaacggctgcgctcccaccaagggcgcggtgctgggcgggcacctgtcggccgcgtcggcgctgctgcggtatcaggcggtggcagagggctctgggctgacatcgctggccgcccctttgggcggagaggggacctcaccagtttttttagtatcgcccacgcccagttccctggccgagtccgcagggccctcc

SEQ ID NO: 25 - protein, 408 aa, Homo sapiens
MNLPRAERPRSTPQRSLRDSDGEDGKIDVLGEEEDEDEVEDEEEEASQKFLEQSLQPGLQVARWGGVALPREHIEGGGPSDPSEFGTEFRAPPRSAAASEDARQPAKPPYSYIALITMAILQSPHKRLTLSGICAFISGRFPYYRRKFPAWQNSIRHNLSLNDCFVKIPREPGHPGKGTYWSLDPASQDMFDNGSFLRRRKRFKRHQLTPGAHLPHPFPLPAAHAALHNPRPGPLLGAPALPQPVPGAYPNTAPGRRPYALLHPHPPRYLLLSAPAYAGAPKKAEGADLATPGTLPVLQPSLGPQPWEEGKGLASPPGGGCISFSIESIMQGVRGAGTGAAQSLSPTAWSYCPLLQRPSSLSDNFAATAAASGGGLRQRLRSHQGRGAGRAPVGRVGAAAVSGGGRGL

SEQ ID NO: 26 - DNA, 2067 bp, Homo sapiens
  source 1..2067: mol_type = genomic DNA
atctttgccggacgttgttgcaaaggagtagaaacaagcagaggaaaacatcccaaagggtaaccactagcgttcctgcttcttgcaacattcatcccaggcttccagctcagcccgccccgggccaggtgatcggccgccacatcccctgcgactgaagcacctgctccgccatgaacctgccaagagctgagcgccctcgctccacaccgcagcgcagcctccgggactccgatggggaagacggtaaaatcgatgtcctgggagaggaggaagatgaagacgaggtggaagacgaggaggaggaggcgagccagaagttcctagagcagtcgctccagccggggctgcaggtggcccggtggggcggggttgcgcttccccgagagcacatcgagggcggcggcccgagcgacccctcagagtttggcaccgagttcagggcaccgccaaggtctgcggcggcctctgaagatgcccggcagccggcaaagcccccctactcgtacatcgcgctcatcaccatggccatcctgcaaagcccgcacaagcgcctcacgctcagcggcatctgcgccttcattagtggccgcttcccctactaccgccgcaagttccccgcctggcagaacagcatccgccacaacctctcgctgaacgactgcttcgtcaagatcccccgcgagccgggccacccaggcaagggcacctactggagcctggaccccgcctcccaggacatgttcgacaatggcagctttctccggcgtaggaagcgtttcaagcgccaccaactgaccccgggagcccacctgccccaccccttccctctacctgctgcacacgccgccctgcacaacccccgcccaggccctctgcttggggcccctgccctgccgcagccagtcccgggggcctaccccaacaccgcccccgggagacgcccttacgctctgctgcacccgcatcctcctcgctacctactgctctcggcccccgcctatgccggggcaccgaagaaagcagaaggcgcggacctggcgacccccggcacccttcccgtgctgcagccctcacttggtcctcagccttgggaggagggcaagggtctggcgtcgccaccgggaggcggatgcatctctttcagcattgagagtatcatgcaaggggtcaggggagcgggtacaggggctgcgcagagtttgtccccgaccgcgtggagctactgccccctgctccagcgaccgtcaagcctgtcggacaattttgcagcaacagcagcagcatcaggaggaggactgcgccaacggctgcgctcccaccaagggcgcggtgctgggcgggcacctgtcggccgcgtcggcgctgctgcggtatcaggcggtggcagagggctctaggctgacatcgctggctgcccctttgggcggagaggggacctcaccagtttttttagtatcgcccacgcccagttccctggccaagtccgcagggccctcctagagccaggtgggagtggggagcgatccgcagctgctcactccaccttgcgcggcccatactgggcgtgtgcatctgaatcctgctggagagcaaacacgaacttctgttccctgcaaaatggttagaaagaaacagctggattacgttcctctaaaaaccacctgaacgtaaccttcgcagggcgtcaagtcatcttttcttgccttcggctgtggcttctgtggctttccggatttgcacatttcctggggtactatgaacgtgagtggggtattttgttctggcattagaagaaaaacaagcaagcaaacaaaaacacagcctccgatgccaaacatgttcccccttcttcacttccttggaactggaagtgttattcctaagtctagtgcaaaatgcttctactctctgtgtcttcctgatagggatgtttaatgtaagtaggatattaatttcagaacattgatttcttatctgtgtgtctgacgtgccatctttaatgttaaaattaaggtgttaaaattaagcctagttatatagacgaaataaaatgctaagtcacta

SEQ ID NO: 27 - protein, 359 aa, Homo sapiens
MNLPRAERLRSTPQRSLRDSDGEDGKIDVLGEEEDEDEVEDEEEAASQQFLEQSLQPGLQVARWGGVALPREHIEGGGGPSDPSEFGTKFRAPPRSAAASEDARQPAKPPYSYIALITMAILQNPHKRLTLSGICAFISGRFPYYRRKFPAWQNSIRHNLSLNDCFVKIPREPGHPGKGNYWSLDPASQDMFDNGSFLRRRKRFKRHQLTPGAHLPHPFPLPAAHAALHNPRPGPLLGAPAPPQPVPGAYPNTAPGRRPYALLHPHPLRYLLLSAPVYAGAPKKAEGAALATPAPFPCCSPHLVLSLGRRARVWRRHREADASLSALRVLCKGSRTAPTAALPPRARCWAGTCRPRRPC

SEQ ID NO: 28 - DNA, 2218 bp, Homo sapiens
  source 1..2218: mol_type = genomic DNA
cattcatcccaggcttccagctcagcccgccccaggccaggtgatcggccgccacatcccctgcgactgaagcacctgctcctccatgaacctgccaagagctgagcgccttcgctccacaccgcagcgcagcctccgggactccgatggggaagacggtaaaatcgatgtcctgggagaggaggaagatgaagacgaggtggaagacgaggaggaggcggcgagccagcagttcctagagcagtcgctccagccggggctgcaggtggcccggtggggcggggttgcgcttccccgagagcacatcgagggcggcggcggcccgagcgacccctcagagtttggcaccaagttcagggcaccgccaaggtctgcggcggcctctgaagatgcccggcagccggcaaagcccccctactcgtacatcgcgctcatcaccatggccatcctgcaaaacccgcacaagcgcctcacgctcagcggcatctgcgccttcattagtggccgcttcccctactaccgccgcaagttccccgcctggcagaacagcatccgccacaacctctcgctgaacgactgcttcgttaagatcccccgcgagccgggccacccaggcaagggcaactactggagcctggaccccgcctcccaagacatgttcgacaatggcagctttctccggcgtaggaagcgtttcaagcgccaccaactgaccccgggagcccacctgccccaccccttccctctacctgctgcacacgccgccctgcacaacccccgcccaggccctctgcttggggcccctgccccgccgcagccagtcccgggggcctaccccaacaccgcccccgggagacgcccttacgctctgctgcacccgcatcctcttcgctacctactgctctcggcccccgtctatgccggggcaccgaagaaagcagaaggcgcggccctggcgaccccggcacccttcccgtgctgcagccctcacttggtcctcagccttgggaggagggcaagggtctggcgtcgccaccgggaggcggatgcatctctttcagcattgagagtattatgcaaggggtcaggggagcgggtacaggggctgcgcagaatttgtccccgaccgcgtggagctactgccacctgctccagcgaccatcaagcctgttgcatccccagaccgctgcccctttgctgcaagtgtccgccgccgccgctgctcggacaattttgcagcaatagcagcagcatcaggaggaggactgcgccaacggctgcgctcccaccaagggcgcggtgctgggcgggcacctgtcggcctcgtcggccctgctgaggtatcaggcagtggcagagggctctaggctgacatcgctggctgcccctttgggcggagaggggacctcaccagtttttttagtatcgcccacgcccagttccctggccaactccgcagggccctcctagagccaggtgggagtggggagcgacccgcagctgctcactccaccttgcgcggcccatactgggcgtgtgcatctgaatcccgctggagagcaaacacgaacttctgttcgctgcaaaatggttagaaagaaacagctggattacgttcctctaaaaaccacctgaacgtaaccttcgcagggcgtcaagtcatcttttcttgccttcggttgtggcttctatggctgtcccgatttgcgcatttcctggggtactatgaacgtgagtggggtattttgttctggcattaaaagaaaaacaagcaagcaaacaaaaacacagcctccgatgccaaacatgttcccccttcttcacttccttggagctggaagtattattcctaagtctagtgcaaaatgcttctactctctgtgtcttcctgatagggatgtttaatgtaagtaggatattaatttcagaacattgatttcttatctgtgtgtctgacgtgccatctttaatgttaaaattaaggtgttaaaattaagcctagttatatagacgaaataaaatgctaagtcactacactacatcgtttattttctattacatctcattcttccctttctaaatggaactttttaaaacctacgttattttccctcaaacaatttattttcacaattcatatttattatagatagcagaagtaatccattttaatatggcctttaaaaattccaaatatttgaggttgaaaatgtcctgg

SEQ ID NO: 29 - protein, 373 aa, Homo sapiens
MTAESGPPPPQPEVLATVKEERGETAAGAGVPGEATGRGAGGRRRKRPLQRGKPPYSYIALIAMAIAHAPERRLTLGGIYKFITERFPFYRDNPKKWQNSIRHNLTLNDCFLKIPREAGRPGKGNYWALDPNAEDMFESGSFLRRRKRFKRSDLSTYPAYMHDAAAAAAAAAAAAAAAAIFPGAVPAARPPYPGAVYAGYAPPSLAAPPPVYYPAASPGPCRVFGLVPERPLSPELGPAPSGPGGSCAFASAGAPATTTGYQPAGCTGARPANPSAYAAAYAGPDGAYPQGAGSAIFAAAGRLAGPASPPAGGSSGGVETTVDFYGRTSPGQFGALGACYNPGGQLGGASAGAYHARHAAAYPGGIDRFVSAM

SEQ ID NO: 30 - DNA, 1118 bp, Homo sapiens
  source 1..1118: mol_type = genomic DNA
atgactgccgagagcgggccgccgccgccgcagccggaggtgctggctaccgtgaaggaagagcgcggcgagacggcagcaggggccggggtcccaggggaggccacgggccgcggggcgggcgggcggcgccgcaagcgccccctgcagcgcgggaagccgccctacagctacatcgcgctcatcgccatggccatcgcgcacgcgcccgagcgccgcctcacgctgggcggcatctacaagttcatcaccgagcgcttccccttctaccgcgacaaccccaaaaagtggcagaacagcatccgccacaacctcacactcaacgactgcttcctcaagatcccgcgcgaggccggccgcccgggtaagggcaactactgggcgcttgaccccaacgcggaggacatgttcgagagcggcagcttcctgcgccgccgcaagcgcttcaagcgctcggacctctccacctacccggcttacatgcacgacgcggcggctgccgcagccgccgccgccgccgccgccgccgccgccgccatcttcccaggcgcggtgcccgccgcgcgccccccctacccgggcgccgtctatgcaggctacgcgccgccgtcgctggccgcgccgcctccagtctactaccccgcggcgtcgcccggcccttgccgcgtcttcggcctggttcctgagcggccgctcagcccagagctggggcccgcaccgtcggggcccggcggctcttgcgcctttgcctccgccgggcccccgctaccaccaccggctaccagcccgcaggctgcaccggggcccggccggccaacccctccgcctatgcggctgcctacgcgggccccgacggcgcgtacccgcagggcgccggcagtgcgatctttgccgctgctggccgcctggcgggacccgcttcgcccccagcgggcggcagcagtggcggcgtggagaccacggtggacttctacgggcgcacgtcgcccggccagttcggagcgctgggagcctgctacaaccctggcgggcagctcggaggggccagtgcaggcgcctaccatgctcgccatgctgccgcttatcccggtgggatagatcggttcgtgtccgccatg

SEQ ID NO: 31 - protein, 319 aa, Homo sapiens
MAGRSDMDPPAAFSGFPALPAVAPSGPPPSPLAGAEPGREPEEAAAGRGEAAPTPAPGPGRRRRRPLQRGKPPYSYIALIAMALAHAPGRRLTLAAIYRFITERFAFYRDSPRKWQNSIRHNLTLNDCFVKVPREPGNPGKGNYWTLDPAAADMFDNGSFLRRRKRFKRAELPAHAAAAPGPPLPFPYAPYAPAPGPALLVPPPSAGPGPSPPARLFSVDSLVNLQPELAGLGAPEPPCCAAPDAAAAAFPPCAAAASPPLYSQVPDRLVLPATRPGPGPLPAEPLLALAGPAAALGPLSPGEAYLRQPGFASGLERYL

SEQ ID NO: 32 - DNA, 957 bp, Homo sapiens
  source 1..957: mol_type = genomic DNA
atggcggggcgcagcgacatggatccgcccgccgcgttctctggcttccctgccctgccagcggtcgcgccgtcggggccgccgccgtcgccgctcgcaggagccgagccagggcgggagccagaggaggcggcggctggccgcggagaggcggcccccacgcccgcgcccggcccggggcggcggcggcggcggcccctgcagcgcgggaagccgccctactcgtacatcgcgctcatcgccatggctctggcgcacgccccgggccgccgcctcacgctggccgccatctaccgcttcatcaccgaacgctttgccttctaccgcgacagcccgcgcaagtggcagaacagcatccgccacaatctcacgctcaacgactgcttcgtcaaggtgccccgcgagccgggcaacccgggcaagggcaactactggacgctggaccccgcggccgcagacatgttcgacaacggcagcttcctgcggcgccgcaagcgcttcaagcgcgccgagctgcccgcgcacgcggccgcggcgccagggccgccgctccccttcccctacgcgccctacgcgcccgcgcccggccccgcgctgctggtgccgccgccttctgccggaccgggcccctcgccgcccgcgcgtctgttcagcgtcgacagcctggtgaacctgcagccggagctagcggggctgggcgcccccgagccgccctgctgcgccgcgcccgacgccgcagccgcagccttcccgccctgcgctgccgccgcctccccgccactctactcgcaggtccccgaccgcctggtactgcccgcgacgcgccccggccccggcccgctgcccgctgagcccctcctggccttggccgggccggcagccgctctcggcccgctcagccctggggaggcctacctgaggcagccgggcttcgcgtcggggctggagcgctacctg

SEQ ID NO: 33 - protein, 489 aa, Homo sapiens
MLDMGDRKEVKMIPKSSFSINSLVPEAVQNDNHHASHGHHNSHHPQHHHHHHHHHHHPPPPAPQPPPPPQQQQPPPPPPPAPQPPQTRGAPAADDDKGPQQLLLPPPPPPPPAAALDGAKAVGLGGKGEPGGGPGELAPVGPDEKEKGAGAGGEEKKGAGEGGKDGEGGKEGEKKNGKYEKPPFSYNALIMMAIRQSPEKRLTLNGIYEFIMKNFPYYRENKQGWQNSIRHNLSLNMCFVKVPRHYDDPGKGNYWMLDPSSDDVFIGGTTGKLRRRSTTSRAKLAFKRGARLTSTGLTFMDRAGSLYWPMSPFLSLHHPRASSTLSYNGTTSAYPSHPMPYSSVLTQNSLGNNHSFSTANGLSVDRLVNGEIPYATHHLTAAALAASVPCGLSVPCSGTYSLNPCSVNLLAGQTSYFFPHVPHPSMTSQSSTSMSARATSSSTSPQAPSTLPCESLRPSLPSFTTGLSGGLSDYFTHQNQGSSSNPLIH

SEQ ID NO: 34 - DNA, 1467 bp, Homo sapiens
  source 1..1467: mol_type = genomic DNA
atgctggacatgggagataggaaagaggtgaaaatgatccccaagtcctcgttcagcatcaacagcctggtgcccgaggcggtccagaacgacaaccaccacgcgagccacggccaccacaacagccaccacccccagcaccaccaccaccaccaccaccatcaccaccacccgccgccgcccgccccgcaaccgccgccgccgccgcagcagcagcagccgccgccgccgccgcccccggcaccgcagcccccccagacgcggggcgccccggccgccgacgacgacaagggcccccagcagctgctgctcccgccgccgccaccgccaccaccggccgccgccctggacggggctaaagcggtcgggctgggcggcaagggcgagccgggcggcgggccgggggagctggcgcccgtcgggccggacgagaaggagaagggcgccggcgccgggggggaggagaagaagggggcgggcgagggcggcaaggacggggaggggggcaaggagggcgagaagaagaacggcaagtacgagaagccgccgttcagctacaacgcgctcatcatgatggccatccggcagagccccgagaagcggctcacgctcaacggcatctacgagttcatcatgaagaacttcccttactaccgcgagaacaagcagggctggcagaactccatccgccacaatctgtccctcaacatgtgcttcgtgaaggtgccgcgccactacgacgacccgggcaagggcaactactggatgctggacccgtcgagcgacgacgtgttcatcggcggcaccacgggcaagctgcggcgccgctccaccacctcgcgggccaagctggccttcaagcgcggtgcgcgcctcacctccaccggcctcaccttcatggaccgcgccggctccctctactggcccatgtcgcccttcctgtccctgcaccacccccgcgccagcagcactttgagttacaacggcaccacgtcggcctaccccagccaccccatgccctacagctccgtgttgactcagaactcgctgggcaacaaccactccttctccaccgccaacggcctgagcgtggaccggctggtcaacggggagatcccgtacgccacgcaccacctcacggccgccgcgctagccgcctcggtgccctgcggcctgtcggtgccctgctctgggacctactccctcaacccctgctccgtcaacctgctcgcgggccagaccagttactttttcccccacgtcccgcacccgtcaatgacttcgcagagcagcacgtccatgagcgccagggccacgtcctcctccacgtcgccgcaggccccctcgaccctgccctgtgagtctttaagaccctctttgccaagttttacgacgggactgtctgggggactgtctgattatttcacacatcaaaatcaggggtcttcttccaaccctttaatacat

SEQ ID NO: 35 - protein, 365 aa, Homo sapiens
MGPCSGSRLGPPEAESPSQPPKRRKKRYLRHDKPPYTYLAMIALVIQAAPSRRLKLAQIIRQVQAVFPFFREDYEGWKDSIRHNLSSNRCFRKVPKDPAKPQAKGNFWAVDVSLIPAEALRLQNTALCRRWQNGGARGAFAKDLGPYVLHGRPYRPPSPPPPPSEGFSIKSLLGGSGEGAPWPGLAPQSSPVPAGTGNSGEEAVPTPPLPSSERPLWPLCPLPGPTRVEGETVQGGAIGPSTLSPEPRAWPLHLLQGTAVPGGRSSGGHRASLWGQLPTSYLPIYTPNVVMPLAPPPTSCPQCPSTSPAYWGVAPETRGPPGLLCDLDALFQGVPPNKSIYDVWVSHPRDLAAPGPGWLLSWCSL

SEQ ID NO: 36 - DNA, 1095 bp, Homo sapiens
  source 1..1095: mol_type = genomic DNA
atggggccctgcagcggctcccgcctggggcccccagaggcagagtcgccctcccagccccctaagaggaggaagaagaggtacctgcgacatgacaagcccccctacacctacttggccatgatcgccttggtgattcaggccgctccctcccgcagactgaagctggcccagatcatccgtcaggtccaggccgtgttccccttcttcagggaagactacgagggctggaaagactccattcgccacaacctttcctccaaccgatgcttccgcaaggtgcccaaggaccctgcaaagccccaggccaagggcaacttctgggcggtcgacgtgagcctgatcccagctgaggcgctccggctgcagaacaccgccctgtgccggcgctggcagaacggaggtgcgcgtggagccttcgccaaggacctgggcccctacgtgctgcacggccggccataccggccgcccagtcccccgccaccacccagtgagggcttcagcatcaagtccctgctaggagggtccggggagggggcaccctggccggggctagctccacagagcagcccagttcctgcaggcacagggaacagtggggaggaggcggtgcccaccccaccccttccctcttctgagaggcctctgtggcccctctgcccccttcctggccccacgagagtggagggggagactgtgcaggggggagccatcgggccctcaaccctctccccagagcctagggcctggcctctccacttactgcagggcaccgcagttcctgggggacggtccagcgggggacacagggcctccctctgggggcagctgcccacctcctacttgcctatctacactcccaatgtggtaatgcccttggcaccaccacccacctcctgtccccagtgtccgtcaaccagccctgcctactggggggtggcccctgaaacccgagggcccccagggctgctctgcgatctagacgccctcttccaaggggtgccacccaacaaaagcatctacgacgtttgggtcagccaccctcgggacctggcggcccctggcccaggctggctgctctcctggtgcagcctg

SEQ ID NO: 37 - protein, 378 aa, Homo sapiens
MSSFDLPAPSPPRCSPQFPSIGQEPPEMNLYYENFFHPQGVPSPQRPSFEGGGEYGATPNPYLWFNGPTMTPPPYLPGPNASPFLPQAYGVQRPLLPSVSGLGGSDLGWLPIPSQEELMKLVRPPYSYSALIAMAIHGAPDKRLTLSQIYQYVADNFPFYNKSKAGWQNSIRHNLSLNDCFKKVPRDEDDPGKGNYWTLDPNCEKMFDNGNFRRKRKRKSDVSSSTASLALEKTESSLPVDSPKTTEPQDILDGASPGGTTSSPEKRPSPPPSGAPCLNSFLSSMTAYVSGGSPTSHPLVTPGLSPEPSDKTGQNSLTFNSFSPLTNLSNHSGGGDWANPMPTNMLSYGGSVLSQFSPHFYNSVNTSGVLYPREGTEV

SEQ ID NO: 38 - DNA, 1134 bp, Homo sapiens
  source 1..1134: mol_type = genomic DNA
atgagctccttcgacctgccggcgccctccccacctcgctgcagcccccagttccccagcatcggccaggagccccccgagatgaacctctactatgagaacttcttccacccacagggcgtgcccagccctcagcggccctccttcgaggggggcggcgagtatggggccacccccaacccctacctctggttcaacgggcccaccatgaccccgccaccctacctgcccggccccaacgccagccccttcctgccccaggcctatggagtgcagagaccgctgctgcccagcgtgtcggggcttggggggagcgacctgggctggctgcccatcccctcgcaggaggagctgatgaagctggtgcggccaccctattcctactcggctctcatcgccatggccatccacggggcacccgacaagcgcctcactctcagccagatctaccagtacgtggccgacaacttccccttctacaacaagagcaaggccggctggcagaactccatccgccacaacctgtcgctcaacgactgcttcaagaaggtgccccgcgacgaggacgacccgggcaaagggaattactggaccctggaccccaactgtgagaaaatgttcgacaatggaaatttccgcaggaaaaggaagagaaaatcagatgtttcctctagcacagcctccttggccttagagaagacagagagcagtctcccggtggacagccccaagaccacggagcctcaggacatcttggatggagcctcaccagggggcaccaccagctccccagagaagcggccctcccctcccccatcaggcgccccttgccttaacagcttcctttcctctatgacagcctatgtgagcggggggagccccacgagccaccccttggtcacaccaggactgagccctgagcccagtgacaagacggggcagaactcactgaccttcaactccttctccccgctcaccaacctcagcaaccacagcggtgggggtgactgggcgaaccccatgcccaccaacatgctcagctacggaggatctgtgctcagccaattcagccctcacttctacaacagtgtcaacaccagtggtgtcctctaccccagggagggcaccgaggtc

SEQ ID NO: 39 - protein, 420 aa, Homo sapiens
MAESWLRLSGAGPAEEAGPEGGLEEPDALDDSLTSLQWLQEFSILNAKAPALPPGGTDPHGYHQVPGSAAPGSPLAADPACLGQPHTPGKPTSSCTSRSAPPGLQAPPPDDVDYATNPHVKPPYYATLICMAMQASKATKITLSAIYKWITDNFCYFRHADPTWQNSIRHNLSLNKCFIKVPREKDEPGKGGFWRIDPQYAERLLSGAFKKRRLPPVHIHPAFARQAAQEPSAVPRAGPLTVNTEAQQLLREFEEATGEAGWGAGEGRLGHKRKQPLPKRVAKVPRPPSTLLPTPEEQGELEPLKGNFDWEAIFDAGTLGGELGALEALELSPPLSPASHVDVDLTIHGRHIDCPATWGPSVEQAADSLDFDETFLATSFLQHPWDESGSGCLPPEPLFEAGDATLASDLQDWASVGAFL

SEQ ID NO: 40 - DNA, 1263 bp, Homo sapiens
  source 1..1263: mol_type = genomic DNA
atggcggagagctggctgcgcctctcgggagccgggccggcggaggaggccgggccggagggcggcctggaggagcccgacgccctggatgacagcctgaccagcctgcagtggctgcaggaattctccattctcaacgccaaggcccccgccctgcccccggggggcaccgacccccacggctaccaccaggtgccaggttcagcggcgcccgggtcccccctggcggccgaccccgcctgcctggggcagccacacacgccgggcaagcccacgtcgtcgtgcacgtcgcggagcgcgcccccggggctgcaggccccaccccccgacgacgtggactacgccaccaatccgcacgtgaagcctccctactcgtatgccacgctcatctgcatggccatgcaggccagcaaggccaccaagatcaccctgtcggccatctacaagtggatcacggacaacttctgctacttccgccacgcagatcccacctggcagaattcaatccgccacaacctgtctctgaacaagtgcttcatcaaagtgcctcgggagaaggacgaaccaggcaaggggggcttctggcgcattgacccccagtacgcggagcggctactgagcggcgctttcaagaagcggcgactgccccctgtccacatccacccagcctttgcccgccaggccgcgcaggagcccagcgctgtcccccgggccgggccgctgacggtgaataccgaggcccagcagctgctgcgggagttcgaggaggccaccggggaggcgggctggggtgcaggcgagggcaggctggggcataagcgcaaacagccgctgcccaagcgggtggccaaggtcccgcggccccccagcaccctgctgcccaccccggaggagcagggtgagctggaacccctcaaaggcaactttgactgggaggccatcttcgacgccggcactctgggcggggagctgggtgcactggaggccctggagctgagcccgcctctgagccccgcctcacacgtggacgtggacctcaccatccacggccgccacatcgactgccctgccacctgggggccttcggtggagcaggctgccgacagcctggacttcgatgagaccttcctggccacatccttcctgcagcacccctgggacgagagcggcagtggctgcctgcccccggagcccctctttgaggctggggatgccaccctggcctccgacctgcaggactgggccagcgtgggggccttcttg

SEQ ID NO: 41 - protein, 733 aa, Homo sapiens
MAEVGEDSGARALLALRSAPCSPVLCAAAAAAAFPAAAPPPAPAQPQPPPGPPPPPPPPLPPGAIAGAGSSGGSSGVSGDSAVAGAAPALVAAAAASVRQSPGPALARLEGREFEFLMRQPSVTIGRNSSQGSVDLSMGLSSFISRRHLQLSFQEPHFYLRCLGKNGVFVDGAFQRRGAPALQLPKQCTFRFPSTAIKIQFTSLYHKEEAPASPLRPLYPQISPLKIHIPEPDLRSMVSPVPSPTGTISVPNSCPASPRGAGSSSYRFVQNVTSDLQLAAEFAAKAASEQQADTSGGDSPKDESKPPFSYAQLIVQAISSAQDRQLTLSGIYAHITKHYPYYRTADKGWQNSIRHNLSLNRYFIKVPRSQEEPGKGSFWRIDPASEAKLVEQAFRKRRQRGVSCFRTPFGPLSSRSAPASPTHPGLMSPRSGGLQTPECLSREGSPIPHDPEFGSKLASVPEYRYSQSAPGSPVSAQPVIMAVPPRPSSLVAKPVAYMPASIVTSQQPAGHAIHVVQQAPTVTMVRVVTTSANSANGYILTSQGAAGGSHDAAGAAVLDLGSEARGLEEKPTIAFATIPAAGGVIQTVASQMAPGVPGHTVTILQPATPVTLGQHHLPVRAVTQNGKHAVPTNSLAGNAYALTSPLQLLATQASSSAPVVVTRVCEVGPKEPAAAVAATATTTPATATTASASASSTGEPEVKRSRVEEPSGAVTTPAGVIAAAGPQGPGTGE

SEQ ID NO: 42 - DNA, 2199 bp, Homo sapiens
  source 1..2199: mol_type = genomic DNA
atggccgaagtcggcgaggacagcggcgcccgcgccctgctcgcgctgcgctcggcgccctgcagcccagtgctgtgcgccgcagccgccgccgccgccttccccgcggccgcacccccgccggcccccgcgcagccccagcctccgcccgggccgccgccgccgccgccaccgccgctgcctccgggcgcgatcgcgggcgcgggctcctccgggggctcctccggggtatccggggactccgcggtcgcgggcgcggcgccggccctggtggccgcggcggccgcctcggtacggcagagcccggggccggcgctggcgcggctggagggccgcgagttcgagttcctcatgcgccagcccagcgtcaccatcggccgcaactcgtcgcagggctcggtggacttgagcatgggcctgtccagcttcatctcgcggcgccacctgcagctcagcttccaggagccgcacttctacctgcgctgcctcggcaagaacggcgtcttcgtggacggggccttccagagacgcggcgcgcccgccctgcagctgcccaagcagtgtaccttccggtttcccagcacggccatcaagatccagttcacgtcgctctatcacaaagaagaggccccagcctccccgctgcggccactgtacccccagatctcccctctgaagatccacatcccggagccggacctccggagcatggtcagccccgtcccctccccgacgggcaccatcagtgtccccaactcctgcccagccagtccacgcggtgccggctcctccagttaccgctttgtgcagaacgtgacctcggacctgcagctggcagcagagtttgcagcaaaggccgcgtcggagcagcaggcagacacgtctggaggagacagccccaaggatgagtcaaagccgccgttctcctacgcgcagctgatcgtgcaggccatctcctccgcccaggaccggcagctgaccctgagcgggatctacgcccacatcaccaagcattacccctactaccggacggccgacaaaggctggcagaattctatccggcacaacctctctttgaaccgttactttatcaaagtcccacgttcccaggaggagcctgggaaggggtccttttggcgaatagaccctgcctctgaagccaagctcgtggaacaggcattccggaaacggaggcagaggggtgtctcctgcttccgcacccccttcgggcctctgtcctcaaggagcgctccagcttcgcccacacaccccgggctgatgtcccctcgctccggcggcctgcagaccccagagtgcctgtctcgggagggctcccccattccacacgaccctgagtttgggtccaagttagcttctgtcccagagtaccggtattcccaaagcgcacccggctcccccgtcagcgcccagccagtgatcatggccgtgcctccccgaccgtccagcctcgtggccaagcccgtggcctacatgcccgcctccatcgtaacctcacagcagcccgcgggccacgccatccacgtcgtgcagcaggcccccaccgtcaccatggtcagggtggtcaccacatctgccaactcggccaacggatacatcctcaccagccagggcgcggcggggggctcccatgatgcggcgggcgcagccgtgctggacctgggcagcgaggccagaggcctggaggagaaacccaccattgcgtttgccacaatccccgcggctggtggagtcatccagacggtggccagccagatggcccccggggtccccggacacacggtcaccatcctgcagcccgccacacccgtgaccctcgggcagcaccaccttccagtccgggccgtgacccagaacggaaagcatgcggttcccacgaacagtttagccggcaacgcttacgccctcaccagccctttgcagctccttgcgacccaagcgagttcatccgcgccggtggtggtcacccgggtgtgcgaggtggggcccaaggagccagcagcagccgtcgcggccacggccaccaccaccccagccactgccaccaccgcctctgcctccgcctcttccactggagagcccgaggtcaaaaggtcccgggtggaggagcccagtggggctgtaaccacaccggctggagtgatcgcagctgccggcccccaggggccaggcaccggggag

SEQ ID NO: 43 - protein, 345 aa, Homo sapiens
MSHLFDPRLPALAASPMLYLYGPERPGLPLAFAPAAALAASGRAETPQKPPYSYIALIAMAIQDAPEQRVTLNGIYQFIMDRFPFYHDNRQGWQNSIRHNLSLNDCFVKVPREKGRPGKGSYWTLDPRCLDMFENGNYRRRKRKPKPGPGAPEAKRPRAETHQRSAEAQPEAGSGAGGSGPAISRLQAAPAGPSPLLDGPSPPAPLHWPGTASPNEDAGDAAQGAAAVAVGQAARTGDGPGSPLRPASRSSPKSSDKSKSFSIDSILAGKQGQKPPSGDELLGGAKPGPGGRLGASLLAASSSLRPPFNASLMLDPHVQGGFYQLGIPFLSYFPLQVPDTVLHFQ

SEQ ID NO: 44 - DNA, 1035 bp, Homo sapiens
  source 1..1035: mol_type = genomic DNA
atgagtcacctcttcgatccccggctgcctgccctggccgcctcgcccatgctgtatctgtacggtcccgagagacccggcctccctctggccttcgcccccgcggctgctctagctgcctcgggccgggccgagaccccgcagaagcctccctacagctacatcgcgctcatcgccatggcgatccaggacgcgcccgagcagagggtcacgctcaacggcatctaccagttcatcatggaccgcttccccttctaccacgacaaccggcagggctggcagaacagcatccgccacaacctctcgctcaacgactgcttcgtcaaggtgccccgcgagaaagggcggccgggcaagggcagctactggacgctggacccccgctgcctggacatgtttgagaacggcaactaccggcgccggaagaggaagcccaagccgggccccggggccccggaggccaagaggccccgcgccgagacgcaccagcgcagcgcggaggcgcagccggaggcggggagcggggcagggggctcgggccccgcaatctcccgcctgcaggcagcgcccgcgggcccctcgcccctcctggacggcccctctccgccggcgcccctccactggccggggaccgcgtccccgaacgaggacgctggtgacgctgcccagggcgcagcggccgtggcggtcggccaggcagcgcgcacaggggacggcccggggtcccctctgcgccccgcctcccgcagctctccgaagagctccgacaagtccaagagcttcagcatagacagcatcctggcgggaaagcagggccagaagccgccttcaggggacgaactcctagggggtgccaagcctgggcccggcggccgtctgggtgcctcgctcctggccgcctcctccagcctccgtccgcctttcaacgcttccctgatgctcgacccgcatgtccagggcggcttttaccagctcgggatccccttcctctcttatttccccctgcaggttcccgacacggtactccacttccag

SEQ ID NO: 45 - protein, 376 aa, Homo sapiens
MMASYPEPEDAAGALLAPETGRTVKEPEGPPPSPGKGGGGGGGTAPEKPDPAQKPPYSYVALIAMAIRESAEKRLTLSGIYQYIIAKFPFYEKNKKGWQNSIRHNLSLNECFIKVPREGGGERKGNYWTLDPACEDMFEKGNYRRRRRMKRPFRPPPAHFQPGKGLFGAGGAAGGCGVAGAGADGYGYLAPPKYLQSGFLNNSWPLPQPPSPMPYASCQMAAAAAAAAAAAAAAGPGSPGAAAVVKGLAGPAASYGPYTRVQSMALPPGVVNSYNGLGGPPAAPPPPPHPHPHPHAHHLHAAAAPPPAPPHHGAAAPPPGQLSPASPATAAPPAPAPTSAPGLQFACARQPELAMMHCSYWDHDSKTGALHSRLDL

SEQ ID NO: 46 - DNA, 1128 bp, Homo sapiens
  source 1..1128: mol_type = genomic DNA
atgatggccagctaccccgagcccgaggacgcggcgggggccctgctggccccagagaccggtcgcacagtcaaggagccagaagggccgccgccgagcccaggcaagggcggtgggggtggcggcgggacagccccggagaagccggacccggcgcagaagcccccgtactcgtacgtggcgctcatcgccatggcgatccgcgagagcgcggagaagaggctcacgctgtccggcatctaccagtacatcatcgcgaagttcccgttctacgagaagaataagaagggctggcaaaatagcatccgccacaacctcagcctcaacgagtgcttcatcaaggtgccgcgcgagggcggcggcgagcgcaagggcaactactggacgctggacccggcctgcgaagacatgttcgagaagggcaactaccggcgccgccgccgcatgaagaggcccttccggccgccgcccgcgcacttccagcccggcaaggggctcttcggggccggaggcgccgcaggcgggtgcggcgtggcgggcgccggggccgacggctacggctacctggcgccccccaagtacctgcagtctggcttcctcaacaactcgtggccgctaccgcagcctccctcacccatgccctatgcctcctgccagatggcggcagccgcagcggctgcagcagctgcggctgcagccgcgggccccggtagccctggcgcggccgctgtggtcaaggggctggcgggcccggccgcctcgtacgggccgtacacacgcgtgcagagcatggcgctgccccccggcgtagtgaactcgtacaatggcctgggaggcccgccggccgcacccccgcctccgccgcacccccacccgcatccgcacgcacaccatctgcacgcggccgccgcaccgccgcctgccccaccgcaccacggggccgccgcgccgccgccgggccagctcagccctgccagcccagccaccgccgcgcccccggcgcccgcgcccaccagtgcgccgggcctgcagttcgcttgtgcccggcagcccgagctcgccatgatgcattgctcttactgggaccacgacagcaagaccggcgcgctgcattcgcgcctcgatctc

SEQ ID NO: 47 - protein, 763 aa, Homo sapiens
MKTSPRRPLILKRRRLPLPVQNAPSETSEEEPKRSPAQQESNQAEASKEVAESNSCKFPAGIKIINHPTMPNTQVVAIPNNANIHSIITALTAKGKESGSSGPNKFILISCGGAPTQPPGLRPQTQTSYDAKRTEVTLETLGPKPAARDVNLPRPPGALCEQKRETCADGEAAGCTINNSLSNIQWLRKMSSDGLGSRSIKQEMEEKENCHLEQRQVKVEEPSRPSASWQNSVSERPPYSYMAMIQFAINSTERKRMTLKDIYTWIEDHFPYFKHIAKPGWKNSIRHNLSLHDMFVRETSANGKVSFWTIHPSANRYLTLDQVFKPLDPGSPQLPEHLESQQKRPNPELRRNMTIKTELPLGARRKMKPLLPRVSSYLVPIQFPVNQSLVLQPSVKVPLPLAASLMSSELARHSKRVRIAPKVLLAEEGIAPLSSAGPGKEEKLLFGEGFSPLLPVQTIKEEEIQPGEEMPHLARPIKVESPPLEEWPSPAPSFKEESSHSWEDSSQSPTPRPKKSYSGLRSPTRCVSEMLVIQHRERRERSRSRRKQHLLPPCVDEPELLFSEGPSTSRWAAELPFPADSSDPASQLSYSQEVGGPFKTPIKETLPISSTPSKSVLPRTPESWRLTPPAKVGGLDFSPVQTSQGASDPLPDPLGLMDLSTTPLQSAPPLESPQRLLSSEPLDLISVPFGNSSPSDIDVPKPGSPEPQVSGLAANRSLTEGLVLDTMNDSLSKILLDISFPGLDEDPLGPDNINWSQFIPELQ

SEQ ID NO: 48 - DNA, 2289 bp, Homo sapiens
  source 1..2289: mol_type = genomic DNA
atgaaaactagcccccgtcggccactgattctcaaaagacggaggctgccccttcctgttcaaaatgccccaagtgaaacatcagaggaggaacctaagagatcccctgcccaacaggagtctaatcaagcagaggcctccaaggaagtggcagagtccaactcttgcaagtttccagctgggatcaagattattaaccaccccaccatgcccaacacgcaagtagtggccatccccaacaatgctaatattcacagcatcatcacagcactgactgccaagggaaaagagagtggcagtagtgggcccaacaaattcatcctcatcagctgtgggggagccccaactcagcctccaggactccggcctcaaacccaaaccagctatgatgccaaaaggacagaagtgaccctggagaccttgggaccaaaacctgcagctagggatgtgaatcttcctagaccacctggagccctttgcgagcagaaacgggagacctgtgcagatggtgaggcagcaggctgcactatcaacaatagcctatccaacatccagtggcttcgaaagatgagttctgatggactgggctcccgcagcatcaagcaagagatggaggaaaaggagaattgtcacctggagcagcgacaggttaaggttgaggagccttcgagaccatcagcgtcctggcagaactctgtgtctgagcggccaccctactcttacatggccatgatacaattcgccatcaacagcactgagaggaagcgcatgactttgaaagacatctatacgtggattgaggaccactttccctactttaagcacattgccaagccaggctggaagaactccatccgccacaacctttccctgcacgacatgtttgtccgggagacgtctgccaatggcaaggtctccttctggaccattcaccccagtgccaaccgctacttgacattggaccaggtgtttaagccactggacccagggtctccacaattgcccgagcacttggaatcacagcagaaacgaccgaatccagagctccgccggaacatgaccatcaaaaccgaactccccctgggcgcacggcggaagatgaagccactgctaccacgggtcagctcatacctggtacctatccagttcccggtgaaccagtcactggtgttgcagccctcggtgaaggtgccattgcccctggcggcttccctcatgagctcagagcttgcccgccatagcaagcgagtccgcattgcccccaaggtgctgctagctgaggaggggatagctcctctttcttctgcaggaccagggaaagaggagaaactcctgtttggagaagggttttctcctttgcttccagttcagactatcaaggaggaagaaatccagcctggggaggaaatgccacacttagcgagacccatcaaagtggagagccctcccttggaagagtggccctccccggccccatctttcaaagaggaatcatctcactcctgggaggattcgtcccaatctcccaccccaagacccaagaagtcctacagtgggcttaggtccccaacccggtgtgtctcggaaatgcttgtgattcaacacagggagaggagggagaggagccggtctcggaggaaacagcatctactgcctccctgtgtggatgagccggagctgctcttctcagaggggcccagtacttcccgctgggccgcagagctcccgttcccagcagactcctctgaccctgcctcccagctcagctactcccaggaagtgggaggaccttttaagacacccattaaggaaacgctgcccatctcctccaccccgagcaaatctgtcctccccagaacccctgaatcctggaggctcacgcccccagccaaagtagggggactggatttcagcccagtacaaacctcccagggtgcctctgaccccttgcctgaccccctggggctgatggatctcagcaccactcccttgcaaagtgctcccccccttgaatcaccgcaaaggctcctcagttcagaacccttagacctcatctccgtcccctttggcaactcttctccctcagatatagacgtccccaagccaggctccccggagccacaggtttctggccttgcagccaatcgttctctgacagaaggcctggtcctggacacaatgaatgacagcctcagcaagatcctgctggacatcagctttcctggcctggacgaggacccactgggccctgacaacatcaactggtcccagtttattcctgagctacag

SEQ ID NO: 49 - protein, 648 aa, Homo sapiens
MVSLPPPQSDVTLPGPTRLEGERQGDLMQAPGLPGSPAPQSKHAGFSCSSFVSDGPPERTPSLPPHSPRIASPGPEQVQGHCPAGPGPGPFRLSPSDKYPGFGFEEAAASSPGRFLKGSHAPFHPYKRPFHEDVFPEAETTLALKGHSFKTPGPLEAFEEIPVDVAEAEAFLPGFSAEAWCNGLPYPSQEHGPQVLGSEVKVKPPVLESGAGMFCYQPPLQHMYCSSQPPFHQYSPGGGSYPIPYLGSSHYQYQRMAPQASTDGHQPLFPKPIYSYSILIFMALKNSKTGSLPVSEIYNFMTEHFPYFKTAPDGWKNSVRHNLSLNKCFEKVENKSGSSSRKGCLWALNPAKIDKMQEELQKWKRKDPIAVRKSMAKPEELDSLIGDKREKLGSPLLGCPPPGLSGSGPIRPLAPPAGLSPPLHSLHPAPGPIPGKNPLQDLLMGHTPSCYGQTYLHLSPGLAPPGPPQPLFPQPDGHLELRAQPGTPQDSPLPAHTPPSHSAKLLAEPSPARTMHDTLLPDGDLGTDLDAINPSLTDFDFQGNLWEQLKDDSLALDPLVLVTSSPTSSSMPPPQPPPHCFPPGPCLTETGSGAGDLAAPGSGGSGALGDLHLTTLYSAFMELEPTPPTAPAGPSVYLSPSSKPVALA

SEQ ID NO: 50 - DNA, 1944 bp, Homo sapiens
  source 1..1944: mol_type = genomic DNA
atggtgtcgctacccccgccgcagtctgacgtcacgctgccgggccccaccagactggagggcgagcgccaaggggacctcatgcaggcaccgggcctcccaggctcccctgccccacagagtaagcatgccggcttcagctgctcgtcatttgtgtccgacggccctccagagaggacaccctcactgcccccacacagcccccgcattgcgtcaccagggcccgagcaagtccagggccactgcccagccggccccggccctgggcccttcaggctctcaccctcagacaagtatcctggctttggctttgaggaggccgcagcaagcagccctgggcgattcctcaagggcagccacgcgcccttccacccgtacaagcggcctttccatgaggacgtcttcccagaggccgagaccaccctggccctcaaaggacactcctttaagaccccagggccgctggaggccttcgaggagatcccagtggacgtggcggaggccgaggccttcctgcctggcttctcagcagaggcctggtgtaacgggctcccctaccccagccaggagcatggcccccaagtcctgggttcagaggtcaaagtcaagcccccagttctggagagtggtgctgggatgttctgctaccagcctcccttgcagcatatgtactgctcctcccagccccccttccaccagtactcgccaggtggtggcagctaccccataccctacctgggctcctcacactatcagtaccagcgaatggcaccccaggccagcaccgatgggcaccagcctctcttcccaaaacccatctattcctacagcatcctcatcttcatggcccttaagaacagtaaaactgggagccttcccgtcagcgagatctacaattttatgacggagcactttccttacttcaagacagcacccgatggctggaagaattctgtccggcacaacctatccctcaacaagtgcttcgagaaggtggagaacaaatcaggaagttcctcccgcaagggctgcctgtgggccctcaatccggccaagatcgacaagatgcaagaggagctgcaaaaatggaagaggaaagatcccattgctgtgcgcaaaagcatggccaagccagaagagctggacagcctcattggagacaagagagaaaagctgggctccccactcctgggctgtccgccccctgggctgtccggctcaggccccatccggcccctggcacccccagctggcctctccccaccactgcactcactccacccagctccaggccccattcctggcaagaaccccctgcaggacctacttatggggcacacaccctcctgctatgggcagacatacttgcacctctcaccaggcctggcccctcctggacccccgcagccattgttcccacagccggacgggcaccttgagctgcgggcccagccaggcaccccccaggactcgcctctgcctgcccacaccccacccagccacagtgccaagctactggccgagccttccccagccaggactatgcacgacaccctgctgccagatggagaccttggcactgacctggatgccatcaatccctcactcactgacttcgacttccagggaaacctgtgggaacagttgaaggatgatagcttggccctcgaccccctggtactggtgacctcatccccgacatcatcttcgatgccaccaccccagccaccacctcactgcttcccccctgggccctgtctgacagagacaggcagtggggcaggtgacttggcagccccgggcagtggtggctccggggcactgggtgacctgcacctcaccaccctctactctgcctttatggagctggagcccacgccccccacggcccctgcaggcccctctgtgtacctcagccccagctccaagcccgtggccctggca

SEQ ID NO: 51 - protein, 431 aa, Homo sapiens
MGPVIGMTPDKRAETPGAEKIAGLSQIYKMGSLPEAVDAARPKATLVDSESADDELTNLNWLHESTNLLTNFSLGSEGLPIVSPLYDIEGDDVPSFGPACYQNPEKKSATSKPPYSFSLLIYMAIEHSPNKCLPVKEIYSWILDHFPYFATAPTGWKNSVRHNLSLNKCFQKVERSHGKVNGKGSLWCVDPEYKPNLIQALKKQPFSSASSQNGSLSPHYLSSVIKQNQVRNLKESDIDAAAAMMLLNTSIEQGILECEKPLPLKTALQKKRSYGNAFHHPSAVRLQESDSLATSIDPKEDHNYSASSMAAQRCASRSSVSSLSSVDEVYEFIPKNSHVGSDGSEGFHSEEDTDVDYEDDPLGDSGYASQPCAKISEKGQSGKKMRKQTCQEIDEELKEAAGSLLHLAGIRTCLGSLISTAKTQNQKQRKK

SEQ ID NO: 52 - DNA, 1293 bp, Homo sapiens
  source 1..1293: mol_type = genomic DNA
atgggtccagtaattggaatgactccagataagagagctgaaaccccaggagctgaaaagattgcaggattaagccagatttacaaaatgggaagcttgcctgaagctgttgatgctgccaggccgaaggccactctagtggacagtgagtcagcagatgatgaactcacaaacttgaactggcttcatgaaagcactaatcttctaacaaacttcagcctcggaagtgagggtcttccaattgttagtccattgtatgacatagagggagatgatgtgccatcctttggaccagcttgctaccagaacccagaaaaaaaatcagcgacttcaaagcccccatactcctttagtcttctcatttatatggccattgagcactctccaaataaatgtttgcctgtcaaagaaatttatagctggattctggaccattttccatattttgctactgcaccaacaggctggaagaattctgttcgacataatctgtccctgaataaatgttttcagaaagtggaaagaagccatggcaaggttaatggaaaaggttccttatggtgtgttgatccggaatataaacccaatcttatccaggcactgaagaagcaacctttttcttcagcatcttcacaaaatggttctttatcacctcactatttaagctctgtaatcaagcagaaccaggtgcgaaacctcaaagaatctgatattgatgctgctgctgcaatgatgcttttaaatacttctatagaacaaggaattttagaatgtgagaagcctcttcctcttaaaacagcattgcaaaaaaagaggagttacggcaatgcatttcatcatcccagtgctgtacgattacaagagagtgattctttagccaccagcattgatccaaaagaagatcacaattacagtgcaagtagcatggcagcacagcgttgtgcatccaggtctagcgtgtcttctctgtcttctgtggatgaggtatatgaatttatcccaaagaatagtcacgtgggaagtgatggcagtgaaggatttcacagtgaagaagatacagacgttgattatgaagatgatcctcttggagacagtggctatgcatcacagccttgtgcaaaaatctctgaaaaagggcagtcaggcaaaaagatgcgaaaacagacatgtcaagaaattgatgaggagctcaaagaggcagctggatctctgctccaccttgctggaattcgtacatgtttaggttccctaataagtactgcaaagacacaaaatcaaaagcaacggaaaaaa

SEQ ID NO: 53 - protein, 490 aa, Homo sapiens
MGPVMPPSKKPESSGISVSSGLSQCYGGSGFSKALQEDDDLDFSLPDIRLEEGAMEDEELTNLNWLHESKNLLKSFGESVLRSVSPVQDLDDDTPPSPAHSDMPYDARQNPNCKPPYSFSCLIFMAIEDSPTKRLPVKDIYNWILEHFPYFANAPTGWKNSVRHNLSLNKCFKKVDKERSQSIGKGSLWCIDPEYRQNLIQALKKTPYHPHPHVFNTPPTCPQAYQSTSGPPIWPGSTFFKRNGALLQDPDIDAASAMMLLNTPPEIQAGFPPGVIQNGARVLSRGLFPGVRPLPITPIGVTAAMRNGITSCRMRTESEPSCGSPVVSGDPKEDHNYSSAKSSNARSTSPTSDSISSSSSSADDHYEFATKGSQEGSEGSEGSFRSHESPSDTEEDDRKHSQKEPKDSLGDSGYASQHKKRQHFAKARKVPSDTLPLKKRRTEKPPESDDEEMKEAAGSLLHLAGIRSCLNNITNRTAKGQKEQKETTKN

SEQ ID NO: 54 - DNA, 1470 bp, Homo sapiens
  source 1..1470: mol_type = genomic DNA
atgggtccagtcatgcctcccagtaagaagccagaaagctcaggaattagtgtctccagtggactgagtcagtgttacgggggcagcggtttctccaaggcccttcaggaagacgatgacctcgacttttctctgcctgacatccgattagaagagggggccatggaagatgaagagctgaccaacctgaactggctgcacgagagcaagaacttgctgaagagctttggggagtcggtcctcaggagtgtcagccccgtccaggacctggacgatgacacccccccatcccctgcccactctgacatgccctacgatgccaggcagaaccccaactgcaaacccccctactccttcagctgcctcatatttatggccatcgaggactctccaaccaagcgcctgccagtgaaggatatctacaactggatcttggaacattttccgtattttgcaaatgcacctactgggtggaaaaactcagtgagacacaatttatcattgaataagtgttttaagaaagtggacaaagagaggagtcagagtattgggaaagggtcgttgtggtgcatagacccagagtatagacaaaatctaattcaggctttgaaaaagacaccttatcacccacacccacacgtgttcaatacacctcccacctgtcctcaggcatatcaaagcacatcaggtccacccatctggccgggcagtaccttcttcaagagaaatggagcccttctccaagatcctgacattgatgctgccagtgccatgatgcttttgaatactccccctgagatacaagcaggttttcctccaggagtgatccaaaatggagcgcgggtcctgagccgagggctgtttcctggcgtgcggccgctgccaatcactcccattggggtgacagcggccatgaggaatggcatcaccagctgccggatgcggactgagagtgagccatcttgtggctccccagtggtcagcggagaccccaaggaggatcacaactacagcagtgccaagtcctccaacgcccggagcacctcgcccaccagcgactccatctcctcctcctcctcctcagccgacgaccactatgagtttgccaccaaggggagccaggagggcagcgagggcagcgaggggagcttccggagccacgagagccccagcgacacggaagaggacgacaggaagcacagccagaaggagcccaaggattctctgggggacagcgggtacgcatcccagcacaagaagcgccagcacttcgccaaggccaggaaggtccccagcgacacactgcccctcaaaaagagacgcaccgaaaagccccccgagagcgatgatgaggagatgaaagaagcggcagggtccctcctgcacttagcagggatccggtcctgtttgaataacatcaccaatcggacggcaaaggggcagaaagagcaaaaggaaaccacaaaaaat

SEQ ID NO: 55 - protein, 517 aa, Homo sapiens
MIESDTSSIMSGIIRNSGQNHHPSPQEYRLLATTSDDDLPGDLQSLSWLTAVDVPRLQQMASGRVDLGGPCVPHPHPGALAGVADLHVGATPSPLLHGPAGMAPRGMPGLGPITGHRDSMSQFPVGGQPSSGLQDPPHLYSPATQPQFPLPPGAQQCPPVGLYGPPFGVRPPYPQPHVAVHSSQELHPKHYPKPIYSYSCLIAMALKNSKTGSLPVSEIYSFMKEHFPYFKTAPDGWKNSVRHNLSLNKCFEKVENKMSGSSRKGCLWALNLARIDKMEEEMHKWKRKDLAAIHRSMANPEELDKLISDRPESCRRPGKPGEPEAPVLTHATTVAVAHGCLAVSQLPPQPLMTLSLQSVPLHHQVQPQAHLAPDSPAPAQTPPLHALPDLSPSPLPHPAMGRAPVDFINISTDMNTEVDALDPSIMDFALQGNLWEEMKDEGFSLDTLGAFADSPLGCDLGASGLTPASGGSDQSFPDLQVTGLYTAYSTPDSVAASGTSSSSQYLGAQGNKPIALL

SEQ ID NO: 56 - DNA, 1551 bp, Homo sapiens
  source 1..1551: mol_type = genomic DNA
atgatagaaagtgacacctcatccataatgtcaggaattattcgaaactcagggcaaaatcaccacccctctccacaggaatacaggcttctagccaccaccagcgatgatgaccttcccggggacctgcagtcgctgtcgtggctcacggcggtggatgtgcctcggctgcagcagatggcaagtggccgcgtggacctgggtggcccctgcgtgccacatccacacccaggtgccttggctggggtggccgacctgcatgtgggagccactccaagtccccttctccatggcccagcaggcatggccccccgaggcatgccaggtctgggccccataactggccacagagacagcatgagccagttccccgtggggggccagccctcatctggcctgcaggacccgccgcatctgtactcacctgccacccaaccacagttcccgctccccccgggtgcccagcagtgccctcctgtgggcctctatggccccccatttggggtgcggcccccctacccccagccccacgtggctgtgcattcatctcaagaactgcaccccaaacactaccccaagcccatctactcgtacagctgtctgatcgccatggccctgaagaacagcaagacaggcagcctgcctgtgagcgagatctacagcttcatgaaggagcacttcccctacttcaagacggcccccgacgggtggaagaactcggtgcggcacaacctgtctctgaacaagtgcttcgagaaggtggagaacaagatgagcggctcctcccgcaagggctgcctgtgggctctgaacctggcccgcatcgacaagatggaggaggagatgcacaagtggaagaggaaggacctggctgccatccaccggagtatggccaaccctgaggagttggacaagctgatctccgaccggcctgaaagctgccggcgccccggcaaaccgggggaaccagaggcccccgtgctgactcacgccaccacagtggccgtggcgcatggctgcctggctgtctcccagctcccaccccagccactgatgaccctgtccctgcagtcagtccccctgcaccaccaggtccagccccaggcacatcttgctccagactctccagcaccagcccagaccccgccactgcacgccctgccggacctcagccccagcccgctcccccaccccgccatgggaagggctcctgtagacttcatcaacatcagcaccgacatgaacactgaggtggatgccctcgacccgagcatcatggacttcgctctgcaggggaacctgtgggaggagatgaaggatgagggattcagcttggacacactgggcgcctttgcagactccccgcttggctgtgacctgggggcctcaggcctaacccctgcctcgggtggcagcgaccagtccttcccagacttgcaggtgacgggtctctacacagcgtactccactccggacagtgtggctgcatcgggcaccagctcctcctcccagtacctgggtgcacaggggaacaagcctatagccctgctt

SEQ ID NO: 57 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQVSHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 58 - DNA, 1965 bp, Homo sapiens
  source 1..1965: mol_type = genomic DNA
atggccgaggcgcctcaggtggtggagatcgacccggacttcgagccgctgccccggccgcgctcgtgcacctggccgctgcccaggccggagtttagccagtccaactcggccacctccagcccggcgccgtcgggcagcgcggctgccaaccccgacgccgcggcgggcctgccctcggcctcggctgccgctgtcagcgccgacttcatgagcaacctgagcttgctggaggagagcgaggacttcccgcaggcgcccggctccgtggcggcggcggtggcggcggcggccgccgcggccgccaccggggggctgtgcggggacttccagggcccggaggcgggctgcctgcacccagcgccaccgcagcccccgccgcccgggccgctgtcgcagcacccgccggtgccccccgccgccgctgggccgctcgcggggcagccgcgcaagagcagctcgtcccgccgcaacgcgtggggcaacctgtcctacgccgacctcatcaccaaggccatcgagagctcggcggagaagcggctcacgctgtcgcagatctacgagtggatggtcaagagcgtgccctacttcaaggataagggtgacagcaacagctcggcgggctggaagaattcaattcgtcataatctgtccctacacagcaagttcattcgtgtgcagaatgaaggaactggaaaaagttcttggtggatgctcaatccagagggtggcaagagcgggaaatctcctaggagaagagctgcatccatggacaacaacagtaaatttgctaagagccgaagccgagctgccaagaagaaagcatctctccagtctggccaggagggtgctggggacagccctggatcacagttttccaaatggcctgcaagccctggctctcacagcaatgatgactttgataactggagtacatttcgccctcgaactagctcaaatgctagtactattagtgggagactctcacccattatgaccgaacaggatgatcttggagaaggggatgtgcattctatggtgtacccgccatctgccgcaaagatggcctctactttacccagtctgtctgagataagcaatcccgaaaacatggaaaatcttttggataatctcaaccttctctcatcaccaacatcattaactgtttcgacccagtcctcacctggcaccatgatgcagcagacgccgtgctactcgtttgcgccaccaaacaccagtttgaattcacccagcccaaactaccaaaaatatacatatggccaatccagcatgagccctttgccccagatgcctatacaaacacttcaggacaataagtcgagttatggaggtatgagtcagtataactgtgcgcctggactcttgaaggagttgctgacttctgactctcctccccataatgacattatgacaccagttgatcctggggtagcccagcccaacagccgggttctgggccagaacgtcatgatgggccctaattcggtcatgtcaacctatggcagccaggtatctcataacaaaatgatgaatcccagctcccatacccaccctggacatgctcagcagacatctgcagttaacgggcgtcccctgccccacacggtaagcaccatgccccacacctcgggtatgaaccgcctgacccaagtgaagacacctgtacaagtgcctctgccccaccccatgcagatgagtgccctggggggctactcctccgtgagcagctgcaatggctatggcagaatgggccttctccaccaggagaagctcccaagtgacttggatggcatgttcattgagcgcttagactgtgacatggaatccatcattcggaatgacctcatggatggagatacattggattttaactttgacaatgtgttgcccaaccaaagcttcccacacagtgtcaagacaacgacacatagctgggtgtcaggc

SEQ ID NO: 59 - protein, 673 aa, Homo sapiens
MAEAPASPAPLSPLEVELDPEFEPQSRPRSCTWPLQRPELQASPAKPSGETAADSMIPEEEDDEDDEDGGGRAGSAMAIGGGGGSGTLGSGLLLEDSARVLAPGGQDPGSGPATAAGGLSGGTQALLQPQQPLPPPQPGAAGGSGQPRKCSSRRNAWGNLSYADLITRAIESSPDKRLTLSQIYEWMVRCVPYFKDKGDSNSSAGWKNSIRHNLSLHSRFMRVQNEGTGKSSWWIINPDGGKSGKAPRRRAVSMDNSNKYTKSRGRAAKKKAALQTAPESADDSPSQLSKWPGSPTSRSSDELDAWTDFRSRTNSNASTVSGRLSPIMASTELDEVQDDDAPLSPMLYSSSASLSPSVSKPCTVELPRLTDMAGTMNLNDGLTENLMDDLLDNITLPPSQPSPTGGLMQRSSSFPYTTKGSGLGSPTSSFNSTVFGPSSLNSLRQSPMQTIQENKPATFSSMSHYGNQTLQDLLTSDSLSHSDVMMTQSDPLMSQASTAVSAQNSRRNVMLRNDPMMSFAAQPNQGSLVNQNLLHHQHQTQGALGGSRALSNSVSNMGLSESSSLGSAKHQQQSPVSQSMQTLSDSLSGSSLYSTSANLPVMGHEKFPSDLDLDMFNGSLECDMESIIRSELMDADGLDFNFDSLISTQNVVGLNVGNFTGAKQASSQSWVPG

SEQ ID NO: 60 - DNA, 2019 bp, Homo sapiens
  source 1..2019: mol_type = genomic DNA
atggcagaggcaccggcttccccggccccgctctctccgctcgaagtggagctggacccggagttcgagccccagagccgtccgcgatcctgtacgtggcccctgcaaaggccggagctccaagcgagccctgccaagccctcgggggagacggccgctgactccatgatccccgaggaggaggacgatgaagacgacgaggacggcgggggacgggccggctcggccatggcgatcggcggcggcggcgggagcggcacgctgggctccgggctgctccttgaggactcggcccgggtgctggcacccggagggcaagaccccgggtctgggccagccaccgcggcgggcgggctgagcgggggtacacaggcgctgctgcagcctcagcaaccgctgccaccgccgcagccgggggcggctgggggctccgggcagccgaggaaatgttcgtcgcggcggaacgcctggggaaacctgtcctacgcggacctgatcacccgcgccatcgagagctccccggacaaacggctcactctgtcccagatctacgagtggatggtgcgttgcgtgccctacttcaaggataagggcgacagcaacagctctgccggctggaagaactccatccggcacaacctgtcactgcatagtcgattcatgcgggtccagaatgagggaactggcaagagctcttggtggatcatcaaccctgatggggggaagagcggaaaagccccccggcggcgggctgtctccatggacaatagcaacaagtataccaagagccgtggccgcgcagccaagaagaaggcagccctgcagacagcccccgaatcagctgacgacagtccctcccagctctccaagtggcctggcagccccacgtcacgcagcagtgatgagctggatgcgtggacggacttccgttcacgcaccaattctaacgccagcacagtcagtggccgcctgtcgcccatcatggcaagcacagagttggatgaagtccaggacgatgatgcgcctctctcgcccatgctctacagcagctcagccagcctgtcaccttcagtaagcaagccgtgcacggtggaactgccacggctgactgatatggcaggcaccatgaatctgaatgatgggctgactgaaaacctcatggacgacctgctggataacatcacgctcccgccatcccagccatcgcccactgggggactcatgcagcggagctctagcttcccgtataccaccaagggctcgggcctgggctccccaaccagctcctttaacagcacggtgttcggaccttcatctctgaactccctacgccagtctcccatgcagaccatccaagagaacaagccagctaccttctcttccatgtcacactatggtaaccagacactccaggacctgctcacttcggactcacttagccacagcgatgtcatgatgacacagtcggaccccttgatgtctcaggccagcaccgctgtgtctgcccagaattcccgccggaacgtgatgcttcgcaatgatccgatgatgtcctttgctgcccagcctaaccagggaagtttggtcaatcagaacttgctccaccaccagcaccaaacccagggcgctcttggtggcagccgtgccttgtcgaattctgtcagcaacatgggcttgagtgagtccagcagccttgggtcagccaaacaccagcagcagtctcctgtcagccagtctatgcaaaccctctcggactctctctcaggctcctccttgtactcaactagtgcaaacctgcccgtcatgggccatgagaagttccccagcgacttggacctggacatgttcaatgggagcttggaatgtgacatggagtccattatccgtagtgaactcatggatgctgatgggttggattttaactttgattccctcatctccacacagaatgttgttggtttgaacgtggggaacttcactggtgctaagcaggcctcatctcagagctgggtgccaggc

SEQ ID NO: 61 - protein, 505 aa, Homo sapiens
MDPGNENSATEAAAIIDLDPDFEPQSRPRSCTWPLPRPEIANQPSEPPEVEPDLGEKVHTEGRSEPILLPSRLPEPAGGPQPGILGAVTGPRKGGSRRNAWGNQSYAELISQAIESAPEKRLTLAQIYEWMVRTVPYFKDKGDSNSSAGWKNSIRHNLSLHSKFIKVHNEATGKSSWWMLNPEGGKSGKAPRRRAASMDSSSKLLRGRSKAPKKKPSVLPAPPEGATPTSPVGHFAKWSGSPCSRNREEADMWTTFRPRSSSNASSVSTRLSPLRPESEVLAEEIPASVSSYAGGVPPTLNEGLELLDGLNLTSSHSLLSRSGLSGFSLQHPGVTGPLHTYSSSLFSPAEGPLSAGEGCFSSSQALEALLTSDTPPPPADVLMTQVDPILSQAPTLLLLGGLPSSSKLATGVGLCPKPLEAPGPSSLVPTLSMIAPPPVMASAPIPKALGTPVLTPPTEAASQDRMPQDLDLDMYMENLECDMDNIISDLMDEGEGLDFNFEPDP

SEQ ID NO: 62 - DNA, 1515 bp, Homo sapiens
  source 1..1515: mol_type = genomic DNA
atggatccggggaatgagaattcagccacagaggctgccgcgatcatagacctagatcccgacttcgaaccccagagccgtccccgctcctgcacctggccccttccccgaccagagatcgctaaccagccgtccgagccgcccgaggtggagccagatctgggggaaaaggtacacacggaggggcgctcagagccgatcctgttgccctctcggctcccagagccggccgggggcccccagcccggaatcctgggggctgtaacaggtcctcggaagggaggctcccgccggaatgcctggggaaatcagtcatatgcagaactcatcagccaggccattgaaagcgccccggagaagcgactgacacttgcccagatctacgagtggatggtccgtactgtaccctacttcaaggacaagggtgacagcaacagctcagcaggatggaagaactcgatccgccacaacctgtccctgcacagcaagttcatcaaggttcacaacgaggccaccggcaaaagctcttggtggatgctgaaccctgagggaggcaagagcggcaaagccccccgccgccgggccgcctccatggatagcagcagcaagctgctccggggccgcagtaaagcccccaagaagaaaccatctgtgctgccagctccacccgaaggtgccactccaacgagccctgtcggccactttgccaagtggtcaggcagcccttgctctcgaaaccgtgaagaagccgatatgtggaccaccttccgtccacgaagcagttcaaatgccagcagtgtcagcacccggctgtcccccttgaggccagagtctgaggtgctggcggaggaaataccagcttcagtcagcagttatgcagggggtgtccctcccaccctcaatgaaggtctagagctgttagatgggctcaatctcacctcttcccattccctgctatctcggagtggtctctctggcttctctttgcagcatcctggggttaccggccccttacacacctacagcagctcccttttcagcccagcagaggggcccctgtcagcaggagaagggtgcttctccagctcccaggctctggaggccctgctcacctctgatacgccaccaccccctgctgacgtcctcatgacccaggtagatcccattctgtcccaggctccgactcttctgttgctgggggggcttccttcctccagtaagctggccacgggcgtcggcctgtgtcccaagcccctagaggctccaggccccagcagtctggttcccaccctttctatgatagcaccacctccagtcatggcaagtgcccccatccccaaggctctggggactcctgtgctcacaccccctactgaagctgcaagccaagacagaatgcctcaggatctagatcttgatatgtatatggagaacctggagtgtgacatggataacatcatcagtgacctcatggatgagggcgagggactggacttcaactttgagccagatccc

SEQ ID NO: 63 - protein, 492 aa, Homo sapiens
MAAKLRAHQVDVDPDFAPQSRPRSCTWPLPQPDLAGDEDGALGAGVAEGAEDCGPERRATAPAMAPAPPLGAEVGPLRKAKSSRRNAWGNLSYADLITKAIESAPDKRLTLSQIYDWMVRYVPYFKDKGDSNSSAGWKNSIRHNLSLHTRFIRVQNEGTGKSSWWMLNPEGGKTGKTPRRRAVSMDNGAKFLRIKGKASKKKQLQAPERSPDDSSPSAPAPGPVPAAAKWAASPASHASDDYEAWADFRGGGRPLLGEAAELEDDEALEALAPSSPLMYPSPASALSPALGSRCPGELPRLAELGGPLGLHGGGGAGLPEGLLDGAQDAYGPRAAPRPGPVLGAPGELALAGAAAAYPGKGAAPYAPPAPSRSALAHPISLMTLPGEAGAAGLAPPGHAAAFGGPPGGLLLDALPGPYAAAAAGPLGAAPDRFPADLDLDMFSGSLECDVESIILNDFMDSDEMDFNFDSALPPPPPGLAGAPPPNQSWVPG

SEQ ID NO: 64 - DNA, 1476 bp, Homo sapiens
  source 1..1476: mol_type = genomic DNA
atggctgcgaagctgcgagcgcatcaggtggacgtggacccggacttcgcgccgcagagccggccgcgctcgtgtacctggcccctgccgcagcctgacttggccggcgacgaggacggagcgctgggcgcaggggtggccgagggcgccgaggactgcgggccggagcgccgggctacggccccggcgatggccccagcgccgcccctgggcgcggaggtcggaccgctgcggaaagcgaagagctctcggcggaacgcgtgggggaacctgtcctacgccgacctcatcaccaaagccatcgagagcgccccggacaagcggctcacgctctcgcagatctacgactggatggtccgttacgtgccctacttcaaggataaaggcgacagcaacagctcggccggctggaagaactccatccggcacaacctgtcgctgcacacccgtttcatccgcgtgcagaacgagggcaccggcaagagttcgtggtggatgctgaaccccgagggcggaaagacagggaagaccccgcggcgcagggccgtgtccatggacaacggggccaagttcctgcgcatcaagggcaaggcgagcaagaagaagcagctgcaggcgcccgagcgaagcccggacgacagctccccgagtgcgcccgccccggggccggtgcctgccgcagccaagtgggccgccagccccgcctcgcacgccagcgacgactacgaggcttgggccgacttccgcggcggcgggagacccctgctcggggaggcggccgagctggaggacgacgaggccctggaggccctggcgccatcatcgccgctcatgtacccaagccccgccagcgcgctgtcgccggcgctgggctcgcgctgtccgggtgagctgccccgcctggccgagctgggaggcccgctgggcctgcacggcggcggcggcgcggggctgcccgagggcctgctggacggcgcgcaggacgcgtacgggccgcgggccgcgcccaggcccggcccggtgctgggtgcgccgggggagctggcgctggcgggcgcagccgccgcctaccccggcaaaggggcggccccgtacgcgccgcccgcgccctcgcgcagtgccttagcccaccccatcagccttatgacgctgcccggcgaggcgggcgccgcgggcctggcaccgccgggccacgccgccgccttcgggggcccgcccggcggcctcctgctggacgctctgccggggccctacgctgccgccgccgccgggccgctgggcgccgcgcccgaccgcttcccggccgacctggacctcgacatgttcagcgggagcctcgagtgcgacgtggagtccatcatcctcaacgacttcatggacagcgacgaaatggacttcaacttcgattcggccctgcctccgccgccgccgggcctggccggggccccgccccccaaccagagctgggtgccgggc

SEQ ID NO: 65 - protein, 677 aa, Homo sapiens
MMQESGTETKSNGSAIQNGSGGSNHLLECGGLREGRSNGETPAVDIGAADLAHAQQQQQQALQVARQLLLQQQQQQQVSGLKSPKRNDKQPALQVPVSVAMMTPQVITPQQMQQILQQQVLSPQQLQVLLQQQQALMLQQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLSAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 66 - DNA, 2031 bp, Homo sapiens
  source 1..2031: mol_type = genomic DNA
atgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcagtgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggag

SEQ ID NO: 67 - protein, 715 aa, Homo sapiens
MMQESATETISNSSMNQNGMSTLSSQLDAGSRDGRSSGDTSSEVSTVELLHLQQQQALQAARQLLLQQQTSGLKSPKSSDKQRPLQVPVSVAMMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPLNLVSSVTMSKNMLETSPQSLPQTPTTPTAPVTPITQGPSVITPASVPNVGAIRRRHSDKYNIPMSSEIAPNYEFYKNADVRPPFTYATLIRQAIMESSDRQLTLNEIYSWFTRTFAYFRRNAATWKNAVRHNLSLHKCFVRVENVKGAVWTVDEVEYQKRRSQKITGSPTLVKNIPTSLGYGAALNASLQAALAESSLPLLSNPGLINNASSGLLQAVHEDLNGSLDHIDSNGNSSPGCSPQPHIHSIHVKEEPVIAEDEDCPMSLVTTANHSPELEDDREIEEEPLSEDLE

SEQ ID NO: 68 - DNA, 2145 bp, Homo sapiens
  source 1..2145: mol_type = genomic DNA
atgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatacattcaatccacgtcaaggaagagccagtgattgcagaggatgaagactgcccaatgtccttagtgacaacagctaatcacagtccagaattagaagacgacagagagattgaagaagagcctttatctgaagatctggaa

SEQ ID NO: 69 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 70 - DNA, 1293 bp, Homo sapiens
  source 1..1293: mol_type = genomic DNA
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccc

SEQ ID NO: 71 - protein, 678 aa, Homo sapiens
MMVESASETIRSAPSGQNGVGSLSGQADGSSGGATGTTASGTGREVTTGADSNGEMSPAELLHFQQQQALQVARQFLLQQASGLSSPGNNDSKQSASAVQVPVSVAMMSPQMLTPQQMQQILSPPQLQALLQQQQALMLQQEYYKKQQEQLHLQLLTQQQAGKPQPKEALGNKQLAFQQQLLQMQQLQQQHLLNLQRQGLVSLQPNQASGPLQTLPQAAVCPTDLPQLWKGEGAPGQPAEDSVKQEGLDLTGTAATATSFAAPPKVSPPLSHHTLPNGQPTVLTSRRDSSSHEETPGSHPLYGHGECKWPGCETLCEDLGQFIKHLNTEHALDDRSTAQCRVQMQVVQQLEIQLAKESERLQAMMAHLHMRPSEPKPFSQPLNPVPGSSSFSKVTVSAADSFPDGLVHPPTSAAAPVTPLRPPGLGSASLHGGGPARRRSSDKFCSPISSELAQNHEFYKNADVRPPFTYASLIRQAILETPDRQLTLNEIYNWFTRMFAYFRRNTATWKNAVRHNLSLHKCFVRVENVKGAVWTVDEREYQKRRPPKMTGSPTLVKNMISGLSYGALNASYQAALAESSFPLLNSPGMLNPGSASSLLPLSHDDVGAPVEPLPSNGSSSPPRLSPPQYSHQVQVKEEPAEAEEDRQPGPPLGAPNPSASGPPEDRDLEEELPGEELS

SEQ ID NO: 72 - DNA, 2034 bp, Homo sapiens
  source 1..2034: mol_type = genomic DNA
atgatggtggaatctgcctcggagacaatcaggtcggctccatctggtcagaatggcgtgggcagcctctctgggcaagcagatggcagcagcggcggggccacagggacaactgcaagtggcacgggcagggaagtgaccacgggtgcagacagcaatggtgagatgagtcccgcagagctgctgcacttccagcagcaacaggctctccaagtggcccggcagttcctgctgcagcaggcctcaggcctgagctccccagggaacaatgacagcaaacagtctgcctctgctgtgcaggtgcctgtgtcggtggccatgatgtcgccgcagatgcttaccccgcaacagatgcagcagatcctgtcgcccccgcagctgcaggccttgctccagcagcagcaagccctcatgctccagcaggagtactacaagaagcagcaggagcagctccacctgcagctcctcacccagcagcaggctgggaaaccgcagcccaaagaggcactggggaacaagcagctggccttccagcagcagctcctgcaaatgcaacagttgcagcagcagcacctgctcaacctgcagaggcaggggctggtcagcctgcagcccaaccaagcctcggggcccctccagacccttccgcaagcagctgtttgcccaacagacctgccccagctgtggaagggcgagggtgcccccgggcagcctgccgaggacagcgtcaagcaggaggggctggacctcactggcacggccgccaccgctacctcgtttgccgctccccccaaggtctcaccccccctctcccaccataccctgcccaacggacagcctactgtgctcacatctcggagagacagctcttcccacgaggagacccccggctcccaccccctgtacggacacggagagtgcaagtggccaggctgtgagaccctgtgtgaagacctgggccagtttatcaaacacctcaacacagagcacgccctggatgaccggagtacagcccagtgccgggtacagatgcaggtggtgcagcagctggagatccagctcgccaaggagagcgagcggctgcaggccatgatggcccacctgcacatgcggccctcggagcccaagcccttcagccagccactgaacccggtccccggctcctcctcattctccaaggtgaccgtctctgcagcagactcattcccagatggtctcgtgcaccccccgacctcggccgcagcccctgtcacccctctacggccccctggcctgggctctgcctccctgcatggtgggggcccagcccgtcggagaagcagtgacaagttctgctcccccatctcctcagagctggcccagaatcatgagttctacaagaacgccgacgtccggccccccttcacctacgcctccctcatccgccaggccatcctggaaacccctgacaggcagctgaccctgaatgagatctataactggttcaccaggatgttcgcctatttccgcagaaacactgccacctggaagaacgccgtgcgccacaacctcagcctgcacaagtgcttcgtccgcgtggagaacgtcaagggtgccgtgtggactgtggacgagcgggagtatcagaagcggagaccgccaaagatgacagggagccccaccctggtgaagaacatgatctctggcctcagctatggagcacttaatgccagctaccaggccgccctggccgagagcagcttccccctcctcaacagccctggcatgctgaaccctggctccgccagcagcctgctgcccctcagccacgatgacgtgggtgcccccgtggagccgctgcccagcaacggcagcagcagccctcctcgcctctccccgccccagtacagccaccaggtgcaggtgaaggaggagccagcagaggcagaggaagacaggcagcccgggcctcccctgggcgcccctaaccccagcgcctcggggcctccggaagacagggacctggaggaggagctgccgggagaagaactgtcc

SEQ ID NO: 73 - protein, 403 aa, Homo sapiens
MKLEVFVPRAAHGDKQGSDLEGAGGSDAPSPLSAAGDDSLGSDGDCAANSPAAGGGARDPPGDGEQSAGGGPGAEEAIPAAAAAAVVAEGAEAGAAGPGAGGAGSGEGARSKPYTRRPKPPYSYIALIAMAIRDSAGGRLTLAEINEYLMGKFPFFRGSYTGWRNSVRHNLSLNDCFVKVLRDPSRPWGKDNYWMLNPNSEYTFADGVFRRRRKRLSHRAPVPAPGLRPEEAPGLPAAPPPAPAAPASPRMRSPARQEERASPAGKFSSSFAIDSILRKPFRSRRLRDTAPGTTLQWGAAPCPPLPAFPALLPAAPCRALLPLCAYGAGEPARLGAREAEVPPTAPPLLLAPLPAAAPAKPLRGPAAGGAHLYCPLRLPAALQAASVRRPGPHLPYPVETLLA

SEQ ID NO: 74 - DNA, 1209 bp, Homo sapiens
  source 1..1209: mol_type = genomic DNA
atgaagttggaggtgttcgtccctcgcgcggcccacggggacaagcagggcagtgacctggagggcgcgggcggcagcgacgcgccgtccccgctgtcggcggcgggagacgactccctgggctcagatggggactgcgcggccaacagcccggccgcgggcggcggcgccagagatccgccgggcgacggcgaacagagtgcgggaggcgggccgggcgcggaggaggcgatcccggcagcagctgctgcagcggtggtggcggagggcgcggaggccggggcggcggggccaggcgcgggcggcgcggggagcggcgagggtgcacgcagcaagccatatacgcggcggcccaagcccccctactcgtacatcgcgctcatcgccatggccatccgcgactcggcgggcgggcgcttgacgctggcggagatcaacgagtacctcatgggcaagttcccctttttccgcggcagctacacgggctggcgcaactccgtgcgccacaacctttcgctcaacgactgcttcgtcaaggtgctgcgcgacccctcgcggccctggggcaaggacaactactggatgctcaaccccaacagcgagtacaccttcgccgacggggtcttccgccgccgccgcaagcgcctcagccaccgcgcgccggtccccgcgcccgggctgcggcccgaggaggccccgggcctccccgccgccccgccgcccgcgcccgccgccccggcctcgccccgcatgcgctcgcccgcccgccaggaggagcgcgccagccccgcgggcaagttctccagctccttcgccatcgacagcatcctgcgcaagcccttccgcagccgccgcctcagggacacggcccccgggacgacgcttcagtggggcgccgcgccctgcccgccgctgcccgcgttccccgcgctcctccccgcggcgccctgcagggccctgctgccgctctgcgcgtacggcgcgggcgagccggcgcggctgggcgcgcgcgaggccgaggtgccaccgaccgcgccgcccctcctgcttgcacctctcccggcggcggcccccgccaagccactccgaggcccggcggccggcggcgcgcacctgtactgccccctgcggctgcccgcagccctgcaggcggcctcagtccgccgccctggcccgcacctgccgtacccggtggagacgctcctagcc

SEQ ID NO: 75 - protein, 292 aa, Homo sapiens
MGNELFLAFTTSHLPLAEQKLARYKLRIVKPPKLPLEKKPNPDKDGPDYEPNLWMWVNPNIVYPPGKLEVSGRRKREDLTSTLPSSQPPQKEEDASCSEAAGVESLSQSSSKRSPPRKRFAFSPSTWELTEEEEAEDQEDSSSMALPSPHKRAPLQSRRLRQASSQAGRLWSRPPLNYFHLIALALRNSSPCGLNVQQIYSFTRKHFPFFRTAPEGWKNTVRHNLCFRDSFEKVPVSMQGGASTRPRSCLWKLTEEGHRRFAEEARALASTRLESIQQCMSQPDVMPFLFDL

SEQ ID NO: 76 - DNA, 876 bp, Homo sapiens
  source 1..876: mol_type = genomic DNA
atggggaacgagctctttctggccttcaccacatctcacctccccttagcggagcagaaacttgccaggtataaactccgaattgttaagccaccaaaattacccctagagaaaaaacccaaccctgataaggatggtccagattatgagcccaacctctggatgtgggtaaatcccaacattgtgtatccccctggaaagctggaggtctcaggacgtaggaagagggaggacctgacaagcacactcccctcctctcagccaccccagaaggaggaagatgccagctgctcagaggccgcaggggtggaatcactgtcccagtcctccagcaagcggtctccccctcggaagcggtttgccttttcccccagcacctgggagctcacagaagaggaggaggctgaggaccaggaagacagctcctctatggctctcccatcccctcacaaaagggcccccctccagagtcggaggcttcggcaagccagcagccaggcggggaggctctggtcccggccccctctcaattacttccacctaattgccctggcattaagaaacagttccccctgtggcctcaacgtgcaacagatctacagtttcactcgaaagcacttcccctttttccggacggccccggaaggctggaagaatactgtccgtcacaatctctgttttcgagacagctttgagaaagtgcctgtcagcatgcagggcggggccagcacacggcctcgatcttgcctctggaagttgaccgaggagggacaccgccgctttgcggaggaggcccgcgccttggcttccactcggctagaaagtatccaacagtgcatgagccagccagatgtgatgcccttcctctttgatctt

SEQ ID NO: 77 - protein, 311 aa, Homo sapiens
MDLKLKDCEFWYSLHGQVPGLLDWDMRNELFLPCTTDQCSLAEQILAKYRVGVMKPPEMPQKRRPSPDGDGPPCEPNLWMWVDPNILCPLGSQEAPKPSGKEDLTNISPFPQPPQKDEGSNCSEDKVVESLPSSSSEQSPLQKQGIHSPSDFELTEEEAEEPDDNSLQSPEMKCYQSQKLWQINNQEKSWQRPPLNCSHLIALALRNNPHCGLSVQEIYNFTRQHFPFFWTAPDGWKSTIHYNLCFLDSFEKVPDSLKDEDNARPRSCLWKLTKEGHRRFWEETRVLAFAQRERIQECMSQPELLTSLFDL

SEQ ID NO: 78 - DNA, 933 bp, Homo sapiens
  source 1..933: mol_type = genomic DNA
atggacttaaaactaaaagactgtgaattttggtatagtctccatggccaggtcccagggctgctggactgggacatgaggaatgagttatttctgccttgtaccacagaccagtgctctttagctgagcaaatccttgccaaatacagagtcggagtaatgaagcccccagaaatgcctcagaagaggagacccagtcctgatggagatggtcctccctgtgaacccaatctgtggatgtgggtggaccccaatatcctgtgcccccttggcagccaggaggccccaaagcccagtggaaaagaggatctgacaaacatttctcctttccctcagcccccacaaaaagacgaagggtctaactgctcagaggacaaagtggtagagtctctgccatcttcctccagtgagcagtctcctttacagaagcagggtatccattcccccagtgactttgagctcacagaagaggaggctgaggaaccagacgacaactccctccagtcccctgaaatgaaatgttaccagagccagaaactatggcaaatcaacaaccaagagaagtcctggcaaaggccccctctcaattgtagccaccttattgccctagcattaagaaacaacccccactgtggcctcagtgtgcaggagatctacaatttcacccgacagcatttcccctttttctggacagctccggatggctggaagagcaccattcattacaacctctgcttcctggacagctttgagaaggtgccagacagccttaaggatgaagataatgcaagacctcgctcttgcctttggaagctcactaaggaggggcaccgccgcttttgggaggagactcgtgtcttagcctttgctcaaagggagagaatccaagagtgcatgagtcagccagagttgttgacctctctctttgatctt

SEQ ID NO: 79 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 80 - DNA, 2478 bp, Homo sapiens
  source 1..2478: mol_type = genomic DNA
atggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaac

SEQ ID NO: 81 - protein, 870 aa, Homo sapiens
MTADKEKKRSSSERRKEKSRDAARCRRSKETEVFYELAHELPLPHSVSSHLDKASIMRLAISFLRTHKLLSSVCSENESEAEADQQMDNLYLKALEGFIAVVTQDGDMIFLSENISKFMGLTQVELTGHSIFDFTHPCDHEEIRENLSLKNGSGFGKKSKDMSTERDFFMRMKCTVTNRGRTVNLKSATWKVLHCTGQVKVYNNCPPHNSLCGYKEPLLSCLIIMCEPIQHPSHMDIPLDSKTFLSRHSMDMKFTYCDDRITELIGYHPEELLGRSAYEFYHALDSENMTKSHQNLCTKGQVVSGQYRMLAKHGGYVWLETQGTVIYNPRNLQPQCIMCVNYVLSEIEKNDVVFSMDQTESLFKPHLMAMNSIFDSSGKGAVSEKSNFLFTKLKEEPEELAQLAPTPGDAIISLDFGNQNFEESSAYGKAILPPSQPWATELRSHSTQSEAGSLPAFTVPQAAAPGSTTPSATSSSSSCSTPNSPEDYYTSLDNDLKIEVIEKLFAMDTEAKDQCSTQTDFNELDLETLAPYIPMDGEDFQLSPICPEERLLAENPQSTPQHCFSAMTNIFQPLAPVAPHSPFLLDKFQQQLESKKTEPEHRPMSSIFFDAGSKASLPPCCGQASTPLSSMGGRSNTQWPPDPPLHFGPTKWAVGDQRTEFLGAAPLGPPVSPPHVSTFKTRSAKGFGARGPDVLSPAMVALSNKLKLKRQLEYEEQAFQDLSGGDPPGGSTSHLMWKRMKNLRGGSCPLMPDKPLSANVPNDKFTQNPMRGLGHPLRHLPLPQPPSAISPGENSKSRFPPQCYATQYQDYSLSSAHKVSGMASRLLGPSFESYLLPELTRYDCEVNVPVLGSSTLLQGGDLLRALDQAT

SEQ ID NO: 82 - DNA, 2610 bp, Homo sapiens
  source 1..2610: mol_type = genomic DNA
atgacagctgacaaggagaagaaaaggagtagctcggagaggaggaaggagaagtcccgggatgctgcgcggtgccggcggagcaaggagacggaggtgttctatgagctggcccatgagctgcctctgccccacagtgtgagctcccatctggacaaggcctccatcatgcgactggcaatcagcttcctgcgaacacacaagctcctctcctcagtttgctctgaaaacgagtccgaagccgaagctgaccagcagatggacaacttgtacctgaaagccttggagggtttcattgccgtggtgacccaagatggcgacatgatctttctgtcagaaaacatcagcaagttcatgggacttacacaggtggagctaacaggacatagtatctttgacttcactcatccctgcgaccatgaggagattcgtgagaacctgagtctcaaaaatggctctggttttgggaaaaaaagcaaagacatgtccacagagcgggacttcttcatgaggatgaagtgcacggtcaccaacagaggccgtactgtcaacctcaagtcagccacctggaaggtcttgcactgcacgggccaggtgaaagtctacaacaactgccctcctcacaatagtctgtgtggctacaaggagcccctgctgtcctgcctcatcatcatgtgtgaaccaatccagcacccatcccacatggacatccccctggatagcaagaccttcctgagccgccacagcatggacatgaagttcacctactgtgatgacagaatcacagaactgattggttaccaccctgaggagctgcttggccgctcagcctatgaattctaccatgcgctagactccgagaacatgaccaagagtcaccagaacttgtgcaccaagggtcaggtagtaagtggccagtaccggatgctcgcaaagcatgggggctacgtgtggctggagacccaggggacggtcatctacaaccctcgcaacctgcagccccagtgcatcatgtgtgtcaactacgtcctgagtgagattgagaagaatgacgtggtgttctccatggaccagactgaatccctgttcaagccccacctgatggccatgaacagcatctttgatagcagtggcaagggggctgtgtctgagaagagtaacttcctattcaccaagctaaaggaggagcccgaggagctggcccagctggctcccaccccaggagacgccatcatctctctggatttcgggaatcagaacttcgaggagtcctcagcctatggcaaggccatcctgcccccgagccagccatgggccacggagttgaggagccacagcacccagagcgaggctgggagcctgcctgccttcaccgtgccccaggcagctgccccgggcagcaccacccccagtgccaccagcagcagcagcagctgctccacgcccaatagccctgaagactattacacatctttggataacgacctgaagattgaagtgattgagaagctcttcgccatggacacagaggccaaggaccaatgcagtacccagacggatttcaatgagctggacttggagacactggcaccctatatccccatggacggggaagacttccagctaagccccatctgccccgaggagcggctcttggcggagaacccacagtccaccccccagcactgcttcagtgccatgacaaacatcttccagccactggcccctgtagccccgcacagtcccttcctcctggacaagtttcagcagcagctggagagcaagaagacagagcccgagcaccggcccatgtcctccatcttctttgatgccggaagcaaagcatccctgccaccgtgctgtggccaggccagcacccctctctcttccatggggggcagatccaatacccagtggcccccagatccaccattacattttgggcccacaaagtgggccgtcggggatcagcgcacagagttcttgggagcagcgccgttggggccccctgtctctccaccccatgtctccaccttcaagacaaggtctgcaaagggttttggggctcgaggcccagacgtgctgagtccggccatggtagccctctccaacaagctgaagctgaagcgacagctggagtatgaagagcaagccttccaggacctgagcgggggggacccacctggtggcagcacctcacatttgatgtggaaacggatgaagaacctcaggggtgggagctgccctttgatgccggacaagccactgagcgcaaatgtacccaatgataagttcacccaaaaccccatgaggggcctgggccatcccctgagacatctgccgctgccacagcctccatctgccatcagtcccggggagaacagcaagagcaggttccccccacagtgctacgccacccagtaccaggactacagcctgtcgtcagcccacaaggtgtcaggcatggcaagccggctgctcgggccctcatttgagtcctacctgctgcccgaactgaccagatatgactgtgaggtgaacgtgcccgtgctgggaagctccacgctcctgcaaggaggggacctcctcagagccctggaccaggccacc

SEQ ID NO: 83 - protein, 465 aa, Homo sapiens
MGEMLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSS

SEQ ID NO: 84 - DNA, 1395 bp, Homo sapiens
  source 1..1395: mol_type = genomic DNA
atgggcgagatgctgctgctggcgagatgtctgctgctagtcctcgtctcctcgctgctggtatgctcgggactggcgtgcggaccgggcagggggttcgggaagaggaggcaccccaaaaagctgacccctttagcctacaagcagtttatccccaatgtggccgagaagaccctaggcgccagcggaaggtatgaagggaagatctccagaaactccgagcgatttaaggaactcacccccaattacaaccccgacatcatatttaaggatgaagaaaacaccggagcggacaggctgatgactcagaggtgtaaggacaagttgaacgctttggccatctcggtgatgaaccagtggccaggagtgaaactgcgggtgaccgagggctgggacgaagatggccaccactcagaggagtctctgcactacgagggccgcgcagtggacatcaccacgtctgaccgcgaccgcagcaagtacggcatgctggcccgcctggcggtggaggccggcttcgactgggtgtactacgagtccaaggcacatatccactgctcggtgaaagcagagaactcggtggcggccaaatcgggaggctgcttcccgggctcggccacggtgcacctggagcagggcggcaccaagctggtgaaggacctgagccccggggaccgcgtgctggcggcggacgaccagggccggctgctctacagcgacttcctcactttcctggaccgcgacgacggcgccaagaaggtcttctacgtgatcgagacgcgggagccgcgcgagcgcctgctgctcaccgccgcgcacctgctctttgtggcgccgcacaacgactcggccaccggggagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagc

SEQ ID NO: 85 - protein, 396 aa, Homo sapiens
MALLTNLLPLCCLALLALPAQSCGPGRGPVGRRRYARKQLVPLLYKQFVPGVPERTLGASGPAEGRVARGSERFRDLVPNYNPDIIFKDEENSGADRLMTERCKERVNALAIAVMNMWPGVRLRVTEGWDEDGHHAQDSLHYEGRALDITTSDRDRNKYGLLARLAVEAGFDWVYYESRNHVHVSVKADNSLAVRAGGCFPGNATVRLWSGERKGLRELHRGDWVLAADASGRVVPTPVLLFLDRDLQRRASFVAVETEWPPRKLLLTPWHLVFAARGPAPAPGDFAPVFARRLRAGDSVLAPGGDALRPARVARVAREEAVGVFAPLTAHGTLLVNDVLASCYAVLESHQWAHRAFAPLRLLHALGALLPGGAVQPTGMHWYSRLLYRLAEELLG

SEQ ID NO: 86 - DNA, 1188 bp, Homo sapiens
  source 1..1188: mol_type = genomic DNA
atggctctcctgaccaatctactgcccctgtgctgcttggcacttctggcgctgccagcccagagctgcgggccgggccgggggccggttggccggcgccgctatgcgcgcaagcagctcgtgccgctactctacaagcaatttgtgcccggcgtgccagagcggaccctgggcgccagtgggccagcggaggggagggtggcaaggggctccgagcgcttccgggacctcgtgcccaactacaaccccgacatcatcttcaaggatgaggagaacagtggagccgaccgcctgatgaccgagcgttgtaaggagcgggtgaacgctttggccattgccgtgatgaacatgtggcccggagtgcgcctacgagtgactgagggctgggacgaggacggccaccacgctcaggattcactccactacgaaggccgtgctttggacatcactacgtctgaccgcgaccgcaacaagtatgggttgctggcgcgcctcgcagtggaagccggcttcgactgggtctactacgagtcccgcaaccacgtccacgtgtcggtcaaagctgataactcactggcggtccgggcgggcggctgctttccgggaaatgcaactgtgcgcctgtggagcggcgagcggaaagggctgcgggaactgcaccgcggagactgggttttggcggccgatgcgtcaggccgggtggtgcccacgccggtgctgctcttcctggaccgggacttgcagcgccgggcttcatttgtggctgtggagaccgagtggcctccacgcaaactgttgctcacgccctggcacctggtgtttgccgctcgagggccggcgcccgcgccaggcgactttgcaccggtgttcgcgcgccggctacgcgctggggactcggtgctggcgcccggcggggatgcgcttcggccagcgcgcgtggcccgtgtggcgcgggaggaagccgtgggcgtgttcgcgccgctcaccgcgcacgggacgctgctggtgaacgatgtcctggcctcttgctacgcggttctggagagtcaccagtgggcgcaccgcgcttttgcccccttgagactgctgcacgcgctaggggcgctgctccccggcggggccgtccagccgactggcatgcattggtactctcggctcctctaccgcttagcggaggagctactgggc

SEQ ID NO: 87 - protein, 411 aa, Homo sapiens
MSPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 88 - DNA, 1233 bp, Homo sapiens
  source 1..1233: mol_type = genomic DNA
atgtctcccgcccggctccggccccgactgcacttctgcctggtcctgttgctgctgctggtggtgccggcggcatggggctgcgggccgggtcgggtggtgggcagccgccggcgaccgccacgcaaactcgtgccgctcgcctacaagcagttcagccccaatgtgcccgagaagaccctgggcgccagcggacgctatgaaggcaagatcgctcgcagctccgagcgcttcaaggagctcacccccaattacaatccagacatcatcttcaaggacgaggagaacacaggcgccgaccgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacaggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagcctcacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggaccccgggggagggtgtgcattggtacccccagctgctctaccgcctggggcgtctcctgctagaagagggcagcttccacccactgggcatgtccggggcagggagc

SEQ ID NO: 89 - protein, 436 aa, Homo sapiens
MSSPGTESAGKSLQYRVDHLLSAVENELQAGSEKGDPTERELRVGLEESELWLRFKELTNEMIVTKNGRRMFPVLKVNVSGLDPNAMYSFLLDFVAADNHRWKYVNGEWVPGGKPEPQAPSCVYIHPDSPNFGAHWMKAPVSFSKVKLTNKLNGGGQIMLNSLHKYEPRIHIVRVGGPQRMITSHCFPETQFIAVTAYQNEEITALKIKYNPFAKAFLDAKERSDHKEMMEEPGDSQQPGYSQSGGWLLPGTSTLCPPANPHPQFGGALSLPSTHSCDRYPTLRSHRSSPYPSPYAHRNNSPTYSDNSPACLSMLQSHDNWSSLGMPAHPSMLPVSHNASPPTSSSQYPSLWSVSNGAVTPGSQAAAVSNGLGAQFFRGSPAHYTPLTHPVSAPSSSGSPLYEGAAAATDIVDSQYDAAAQGRLIASWTPVSPPSM

SEQ ID NO: 90 - DNA, 1308 bp, Homo sapiens
  source 1..1308: mol_type = genomic DNA
atgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgcttatcagaacgaggagatcacagctcttaaaattaagtacaatccatttgcaaaagctttccttgatgcaaaggaaagaagtgatcacaaagagatgatggaggaacccggagacagccagcaacctgggtactcccaatcaggggggtggcttcttcctggaaccagcaccctgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatg

SEQ ID NO: 91 - protein, 682 aa, Homo sapiens
MQLEHCLSPSIMLSKKFLNVSSSYPHSGGSELVLHDHPIISTTDNLERSSPLKKITRGMTNQSDTDNFPDSKDSPGDVQRSKLSPVLDGVSELRHSFDGSAADRYLLSQSSQPQSAATAPSAMFPYPGQHGPAHPAFSIGSPSRYMAHHPVITNGAYNSLLSNSSPQGYPTAGYPYPQQYGHSYQGAPFYQFSSTQPGLVPGKAQVYLCNRPLWLKFHRHQTEMIITKQGRRMFPFLSFNISGLDPTAHYNIFVDVILADPNHWRFQGGKWVPCGKADTNVQGNRVYMHPDSPNTGAHWMRQEISFGKLKLTNNKGASNNNGQMVVLQSLHKYQPRLHVVEVNEDGTEDTSQPGRVQTFTFPETQFIAVTAYQNTDITQLKIDHNPFAKGFRDNYDTIYTGCDMDRLTPSPNDSPRSQIVPGARYAMAGSFLQDQFVSNYAKARFHPGAGAGPGPGTDRSVPHTNGLLSPQQAEDPGAPSPQRWFVTPANNRLDFAASAYDTATDFAGNAATLLSYAAAGVKALPLQAAGCTGRPLGYYADPSGWGARSPPQYCGTKSGSVLPCWPNSAAAAARMAGANPYLGEEAEGLAAERSPLPPGAAEDAKPKDLSDSSWIETPSSIKSIDSSDSGIYEQAKRRRISPADTPVSESSSPLKSEVLAQRDCEKNCAKDISGYYGFYSHS

SEQ ID NO: 92 - DNA, 2046 bp, Homo sapiens
  source 1..2046: mol_type = genomic DNA
atgcagctggagcactgcctttctccttctatcatgctctccaagaaatttctcaatgtgagcagcagctacccacattcaggcggatccgagcttgtcttgcacgatcatcccattatctcgaccactgacaacctggagagaagttcacctttgaaaaaaattaccagggggatgacgaatcagtcagatacagacaattttcctgactccaaggactcaccaggggacgtccagagaagtaaactctctcctgtcttggacggggtctctgagcttcgtcacagtttcgatggctctgctgcagatcgctacctcctctctcagtccagccagccacagtctgcggccactgctcccagtgccatgttcccgtaccccggccagcacggaccggcgcaccccgccttctccatcggcagccctagccgctacatggcccaccacccggtcatcaccaacggagcctacaacagcctcctgtccaactcctcgccgcagggataccccacggccggctacccctacccacagcagtacggccactcctaccaaggagctccgttctaccagttctcctccacccagccggggctggtgcccggcaaagcacaggtgtacctgtgcaacaggcccctttggctgaaatttcaccggcaccaaacggagatgatcatcaccaaacagggaaggcgcatgtttccttttttaagttttaacatttctggtctcgatcccacggctcattacaatatttttgtggatgtgattttggcggatcccaatcactggaggtttcaaggaggcaaatgggttccttgcggcaaagcggacaccaatgtgcaaggaaatcgggtctatatgcatccggattcccccaacactggggctcactggatgcgccaagaaatctcttttggaaaattaaaacttacgaacaacaaaggagcttcaaataacaatgggcagatggtggttttacagtccttgcacaagtaccagccccgcctgcatgtggtggaagtgaacgaggacggcacggaggacactagccagcccggccgcgtgcagacgttcactttccctgagactcagttcatcgccgtcaccgcctaccagaacacggatattacacaactgaaaatagatcacaacccttttgcaaaaggatttcgggataattatgacacgatctacaccggctgtgacatggaccgcctgaccccctcgcccaacgactcgccgcgctcgcagatcgtgcccggggcccgctacgccatggccggctctttcctgcaggaccagttcgtgagcaactacgccaaggcccgcttccacccgggcgcgggcgcgggccccgggccgggtacggaccgcagcgtgccgcacaccaacgggctgctgtcgccgcagcaggccgaggacccgggcgcgccctcgccgcaacgctggtttgtgacgccggccaacaaccggctggacttcgcggcctcggcctatgacacggccacggacttcgcgggcaacgcggccacgctgctctcttacgcggcggcgggcgtgaaggcgctgccgctgcaggctgcaggctgcactggccgcccgctcggctactacgccgacccgtcgggctggggcgcccgcagtcccccgcagtactgcggcaccaagtcgggctcggtgctgccctgctggcccaacagcgccgcggccgccgcgcgcatggccggcgccaatccctacctgggcgaggaggccgagggcctggccgccgagcgctcgccgctgccgcccggcgccgccgaggacgccaagcccaaggacctgtccgattccagctggatcgagacgccctcctcgatcaagtccatcgactccagcgactcggggatttacgagcaggccaagcggaggcggatctcgccggccgacacgcccgtgtccgagagttcgtccccgctcaagagcgaggtgctggcccagcgggactgcgagaagaactgcgccaaggacattagcggctactatggcttctactcgcacagc

SEQ ID NO: 93 - protein, 494 aa, Homo sapiens
MHFSTVTRDMEAFTASSLSSLGAAGGFPGAASPGADPYGPREPPPPPRYDPCAAAAPGAPGPPPPPHAYPFAPAAGAATSAAAEPEGPGASCAAAAKAPVKKNAKVAGVSVQLEMKALWDEFNQLGTEMIVTKAGRRMFPTFQVKLFGMDPMADYMLLMDFVPVDDKRYRYAFHSSSWLVAGKADPATPGRVHYHPDSPAKGAQWMKQIVSFDKLKLTNNLLDDNGHIILNSMHRYQPRFHVVYVDPRKDSEKYAEENFKTFVFEETRFTAVTAYQNHRITQLKIASNPFAKGFRDCDPEDWPRNHRPGALPLMSAFARSRNPVASPTQPSGTEKDAAEARREFQRDAGGPAVLGDPAHPPQLLARVLSPSLPGAGGAGGLVPLPGAPGGRPSPPNPELRLEAPGASEPLHHHPYKYPAAAYDHYLGAKSRPAPYPLPGLRGHGYHPHAHPHHHHHPVSPAAAAAAAAAAAAAAANMYSSAGAAPPGSYDYCPR

SEQ ID NO: 94 - DNA, 1482 bp, Homo sapiens
  source 1..1482: mol_type = genomic DNA
atgcacttcagcaccgtcaccagggacatggaagccttcacggccagcagcctgagcagcctgggggccgcggggggcttcccgggcgccgcgtcgcccggcgccgacccgtacggcccgcgcgagcccccgccgccgccgcgctacgacccgtgcgccgccgccgcccccggcgccccgggtccgccgccgccgccgcacgcctacccgtttgcgccggccgccggggccgccaccagcgccgccgccgagcccgagggccccggggccagctgcgcggccgcagccaaggcgccggtgaagaagaacgcgaaggtggccggtgtgagcgtgcagctagagatgaaggcgctgtgggacgagttcaaccagctgggcaccgagatgatcgtcaccaaggccggcaggcggatgtttcccaccttccaagtgaagctcttcggcatggatcccatggccgactatatgctgctcatggacttcgtgccggtggacgataagcgctaccggtacgccttccacagctcctcctggctggtggcggggaaggccgaccctgccacgccaggccgcgtgcactaccacccggactcgcctgccaagggcgcgcagtggatgaagcaaatcgtgtccttcgacaagctcaagctgaccaacaacttactggacgacaacggccacattattctgaattccatgcacagataccagccccgcttccacgtggtctatgtggacccacgcaaagatagcgagaaatatgccgaggagaacttcaaaacctttgtgttcgaggagacacgattcaccgcggtcactgcctaccagaaccatcggatcacgcagctcaagattgccagcaatcccttcgcgaaaggcttccgggactgtgaccctgaggactggccccggaaccaccggcccggcgcactgccgctcatgagcgccttcgcgcgctcgcggaaccccgtggcttccccgacgcagcccagcggcacggagaaagacgcggctgaggcccggcgagaattccagcgcgacgcgggcgggccagcggtgctcggggacccggcgcatcctccgcagctgctggcccgggtgctaagcccctcgctgcccggggccggcggcgccggcggcttagtcccgctgcccggcgcgcccggaggccggcccagtcccccgaaccccgagctgcgcctggaggcgcccggcgcatcggagccgctgcaccaccacccctacaaatatccggccgccgcctacgaccactatctcggggccaagagccggccggcgccctacccgctgcccggcctgcgtggccacggctaccacccgcacgcgcatccgcaccaccaccaccaccccgtgagtccagccgccgcggccgccgccgccgctgccgcagctgccgcggccgccaacatgtactcgtcggccggagccgcgccgcccggctcctacgactattgccccaga

SEQ ID NO: 95 - protein, 712 aa, Homo sapiens
MREPALAASAMAYHPFHAPRPADFPMSAFLAAAQPSFFPALALPPGALAKPLPDPGLAGAAAAAAAAAAAAEAGLHVSALGPHPPAAHLRSLKSLEPEDEVEDDPKVTLEAKELWDQFHKLGTEMVITKSGRRMFPPFKVRVSGLDKKAKYILLMDIVAADDCRYKFHNSRWMVAGKADPEMPKRMYIHPDSPATGEQWMAKPVAFHKLKLTNNISDKHGFTILNSMHKYQPRFHIVRANDILKLPYSTFRTYVFPETDFIAVTAYQNDKITQLKIDNNPFAKGFRDTGNGRREKRKQLTLPSLRLYEEHCKPERDGAESDASSCDPPPAREPPTSPGAAPSPLRLHRARAEEKSCAADSDPEPERLSEERAGAPLGRSPAPDSASPTRLTEPERARERRSPERGKEPAESGGDGPFGLRSLEKERAEARRKDEGRKEAAEGKEQGLAPLVVQTDSASPLGAGHLPGLAFSSHLHGQQFFGPLGAGQPLFLHPGQFTMGPGAFSAMGMGHLLASVAGGGNGGGGGPGTAAGLDAGGLGPAASAASTAAPFPFHLSQHMLASQGIPMPTFGGLFPYPYTYMAAAAAAASALPATSAAAAAAAAAGSLSRSPFLGSARPRLRFSPYQIPVTIPPSTSLLTTGLASEGSKAAGGNSREPSPLPELALRKVGAPSRGALSPSGSAKEAANELQSIQRLVSGLESQRALSPGRESPK

SEQ ID NO: 96 - DNA, 2136 bp, Homo sapiens
  source 1..2136: mol_type = genomic DNA
atgagagagccggcgctggcggccagcgccatggcttaccacccgttccacgcgccacggcccgccgacttccccatgtccgcctttctggcggcggcgcagccctccttcttcccggcactcgcgctgccgcccggcgcgctggccaagccgctgcccgacccgggcctggcgggggcggcggccgcggcggcggcggcggcagcagcggccgaggcggggctgcacgtctcggcactgggcccgcacccgcccgccgcgcatctgcgctccctcaagagcctggagcccgaggacgaggtggaggacgaccccaaggtgacgctggaggccaaggagctgtgggaccagttccacaagctaggcacggagatggtcatcaccaagtccgggaggcggatgttcccccccttcaaggtgcgagtcagcggcctggacaagaaggccaagtatatcctgctgatggacattgtagccgctgacgattgccgctataagttccacaactcgcgctggatggtggcgggcaaggccgaccctgagatgcccaaacgcatgtacatccacccagacagcccagccacgggggagcagtggatggctaagcctgtggccttccacaagctgaagctgaccaacaacatctctgacaagcacggcttcaccatcctaaactccatgcacaagtaccagccgcgcttccacatagtgcgagccaacgacatcctgaagctgccttacagcaccttccgcacctacgtgttcccggagaccgacttcatcgccgtcactgcctaccagaatgacaagatcacacagctgaagatcgacaacaacccgtttgccaagggcttccgggacaccgggaacggccggcgggagaaaaggaagcagctgacgctgccgtctctacgcttgtacgaggagcactgcaaacccgagcgcgatggcgcggagtcagacgcctcgtcgtgcgaccctccccccgcgcgggaaccacccacctccccgggcgcagcgcccagtccgctgcgcctgcaccgggcccgagctgaggagaagtcgtgcgccgcggacagcgacccggagcctgagcggttgagcgaggagcgtgcgggggcgccgctaggccgcagcccggctccagacagcgccagccccactcgcttgaccgaacccgagcgcgcccgggagcggcgtagtcccgagaggggcaaggagccggccgagagcggcggggacggcccgttcggcctgaggagcctggagaaggagcgcgccgaagctcggaggaaggacgaggggcgcaaggaggcggccgagggcaaggagcagggcctggcgccgctggtggtgcagacagacagtgcgtcccccctgggcgccggacacctgcccggcctggccttttccagccacttgcacgggcagcagttctttgggccgctgggagccggccagccgctcttcctgcaccctggacagttcaccatgggccctggcgccttctccgccatgggcatgggtcacctactggcctcggtggcaggcggcggcaacggcggaggtggcgggcctgggaccgccgcggggctggacgcaggcgggctgggtcccgcggccagcgcagcaagcaccgccgcgcccttcccgttccacctctcccagcacatgctggcatctcagggaattccaatgcccactttcggaggcctcttcccctacccctacacctacatggcagcagcagccgcagccgcctcggctttgcccgccactagtgctgcagctgccgccgccgcagccgccggctccctctcccggagccccttcctgggcagtgcccggccccgactgcgtttcagcccctatcagatcccggtcaccatcccgcctagcactagcctcctcaccaccgggctggcctctgagggctccaaggccgctggtggaaacagccgggagcctagccccctgcccgagctggctctccgcaaagtaggggccccatcccgcggtgccctgtcgcccagtggctcggccaaggaggcggccaatgaactgcagagcatccagagactggtgagtgggctggagagccagcgagccctctccccaggccgggagtcgcccaag

SEQ ID NO: 97 - protein, 743 aa, Homo sapiens
MSLSMRDPVIPGTSMAYHPFLPHRAPDFAMSAVLGHQPPFFPALTLPPNGAAALSLPGALAKPIMDQLVGAAETGIPFSSLGPQAHLRPLKTMEPEEEVEDDPKVHLEAKELWDQFHKRGTEMVITKSGRRMFPPFKVRCSGLDKKAKYILLMDIIAADDCRYKFHNSRWMVAGKADPEMPKRMYIHPDSPATGEQWMSKVVTFHKLKLTNNISDKHGFTLAFPSDHATWQGNYSFGTQTILNSMHKYQPRFHIVRANDILKLPYSTFRTYLFPETEFIAVTAYQNDKITQLKIDNNPFAKGFRDTGNGRREKRKQLTLQSMRVFDERHKKENGTSDESSSEQAAFNCFAQASSPAASTVGTSNLKDLCPSEGESDAEAESKEEHGPEACDAAKISTTTSEEPCRDKGSPAVKAHLFAAERPRDSGRLDKASPDSRHSPATISSSTRGLGAEERRSPVREGTAPAKVEEARALPGKEAFAPLTVQTDAAAAHLAQGPLPGLGFAPGLAGQQFFNGHPLFLHPSQFAMGGAFSSMAAAGMGPLLATVSGASTGVSGLDSTAMASAAAAQGLSGASAATLPFHLQQHVLASQGLAMSPFGSLFPYPYTYMAAAAAASSAAASSSVHRHPFLNLNTMRPRLRYSPYSIPVPVPDGSSLLTTALPSMAAAAGPLDGKVAALAASPASVAVDSGSELNSRSSTLSSSSMSLSPKLCAEKEAATSELQSIQRLVSGLEAKPDRSRSASP

SEQ ID NO: 98 - DNA, 2229 bp, Homo sapiens
  source 1..2229: mol_type = genomic DNA
atgagcctctccatgagagatccggtcattcctgggacaagcatggcctaccatccgttcctacctcaccgggcgccggacttcgccatgagcgcggtgctgggtcaccagccgccgttcttccccgcgctgacgctgcctcccaacggcgcggcggcgctctcgctgccgggcgccctggccaagccgatcatggatcaattggtgggggcggccgagaccggcatcccgttctcctccctggggccccaggcgcatctgaggcctttgaagaccatggagcccgaagaagaggtggaggacgaccccaaggtgcacctggaggctaaagaactttgggatcagtttcacaagcggggcaccgagatggtcattaccaagtcgggaaggcgaatgtttcctccatttaaagtgagatgttctgggctggataaaaaagccaaatacattttattgatggacattatagctgctgatgactgtcgttataaatttcacaattctcggtggatggtggctggtaaggccgaccccgaaatgccaaagaggatgtacattcacccggacagccccgctactggggaacagtggatgtccaaagtcgtcactttccacaaactgaaactcaccaacaacatttcagacaaacatggatttactttggccttcccaagtgatcacgctacgtggcaggggaattatagttttggtactcagactatattgaactccatgcacaaataccagccccggttccacattgtaagagccaatgacatcttgaaactcccttatagtacatttcggacatacttgttccccgaaactgaattcatcgctgtgactgcataccagaatgataagataacccagttaaaaatagacaacaacccttttgcaaaaggtttccgggacactggaaatggccgaagagaaaaaagaaaacagctcaccctgcagtccatgagggtgtttgatgaaagacacaaaaaggagaatgggacctctgatgagtcctccagtgaacaagcagctttcaactgcttcgcccaggcttcttctccagccgcctccactgtagggacatcgaacctcaaagatttatgtcccagcgagggtgagagcgacgccgaggccgagagcaaagaggagcatggccccgaggcctgcgacgcggccaagatctccaccaccacgtcggaggagccctgccgtgacaagggcagccccgcggtcaaggctcaccttttcgctgctgagcggccccgggacagcgggcggctggacaaagcgtcgcccgactcacgccatagccccgccaccatctcgtccagcactcgcggcctgggcgcggaggagcgcaggagcccggttcgcgagggcacagcgccggccaaggtggaagaggcgcgcgcgctcccgggcaaggaggccttcgcgccgctcacggtgcagacggacgcggccgccgcgcacctggcccagggccccctgcctggcctcggcttcgccccgggcctggcgggccaacagttcttcaacgggcacccgctcttcctgcaccccagccagtttgccatggggggcgccttctccagcatggcggccgctggcatgggtcccctcctggccacggtttctggggcctccaccggtgtctcgggcctggattccacggccatggcctctgccgctgcggcgcagggactgtccggggcgtccgcggccaccctgcccttccacctccagcagcacgtcctggcctctcagggcctggccatgtcccctttcggaagcctgttcccttacccctacacgtacatggccgcagcggcggccgcctcctctgcggcagcctccagctcggtgcaccgccaccccttcctcaatctgaacaccatgcgcccgcggctgcgctacagcccctactccatcccggtgccggtcccggacggcagcagtctgctcaccaccgccctgccctccatggcggcggccgcggggcccctggacggcaaagtcgccgccctggccgccagcccggcctcggtggcagtggactcgggctctgaactcaacagccgctcctccacgctctcctccagctccatgtccttgtcgcccaaactctgcgcggagaaagaggcggccaccagcgaactgcagagcatccagcggttggttagcggcttggaagccaagccggacaggtcccgcagcgcgtccccg

SEQ ID NO: 99 - protein, 545 aa, Homo sapiens
MLQDKGLSESEEAFRAPGPALGEASAANAPEPALAAPGLSGAALGSPPGPGADVVAAAAAEQTIENIKVGLHEKELWKKFHEAGTEMIITKAGRRMFPSYKVKVTGMNPKTKYILLIDIVPADDHRYKFCDNKWMVAGKAEPAMPGRLYVHPDSPATGAHWMRQLVSFQKLKLTNNHLDPFGHIILNSMHKYQPRLHIVKADENNAFGSKNTAFCTHVFPETSFISVTSYQNHKITQLKIENNPFAKGFRGSDDSDLRVARLQSKEYPVISKSIMRQRLISPQLSATPDVGPLLGTHQALQHYQHENGAHSQLAEPQDLPLSTFPTQRDSSLFYHCLKRRDGTRHLDLPCKRSYLEAPSSVGEDHYFRSPPPYDQQMLSPSYCSEVTPREACMYSGSGPEIAGVSGVDDLPPPPLSCNMWTSVSPYTSYSVQTMETVPYQPFPTHFTATTMMPRLPTLSAQSSQPPGNAHFSVYNQLSQSQVRERGPSASFPRERGLPQGCERKPPSPHLNAANEFLYSQTFSLSRESSLQYHSGMGTVENWTDG

SEQ ID NO: 100 - DNA, 1635 bp, Homo sapiens
  source 1..1635: mol_type = genomic DNA
atgctgcaggataagggcctgtccgagagcgaggaggccttccgggccccgggcccagcgctcggagaggccagcgcagccaacgcccccgagcccgcgctggcagcgccgggcctcagcggagccgcgctaggcagccccccgggacccggggccgacgtcgtcgccgccgccgccgcggagcagaccatcgagaacatcaaggtggggctgcatgagaaggagctctggaagaagttccacgaggcgggcaccgagatgatcatcactaaggctggcaggaggatgttccccagctacaaggtaaaagtcacaggcatgaaccccaagaccaagtatatcctgctgattgacattgtccctgccgatgaccatcgctacaagttctgtgacaacaaatggatggtggcagggaaggctgagccagccatgccaggaaggctgtatgtccacccggattctcctgccacaggagcccactggatgcggcagctggtctccttccagaagctgaagctgacaaacaaccacctggacccctttggccatatcatcctcaactctatgcacaagtaccagccgcggctccacatcgttaaggctgatgagaacaatgctttcggctccaaaaacactgctttctgcacccacgtgttcccagagacctccttcatctctgtgacctcctaccagaatcacaagatcacccagctgaaaattgagaacaacccttttgccaagggattccggggcagtgatgacagtgacctgcgtgtggcccgactgcagagcaaagaataccccgtgatttccaaaagcatcatgaggcagaggctcatctccccccagctctcagccacaccggacgtgggccccctgctcggcacccaccaggcactccagcactaccagcacgagaacggggcacactcacagctcgcggagccgcaggacctgcccctcagcacctttcccacccagagggactcaagcctcttctatcactgcctgaaaagacgagacggtacccgccacctggacttaccttgcaagcgatcctatctggaagccccctcttcggtgggggaggatcactatttccgttccccccctccctacgaccagcaaatgctgagcccctcctactgcagtgaggtgacccccagagaagcatgtatgtactcaggttcagggcccgagattgccggggtgtctggggtggacgacctgcccccacctccgctgagctgtaacatgtggacttcagtgtcgccgtacaccagctatagcgtgcagacgatggagactgtgccgtaccagcccttccccacgcacttcaccgccaccaccatgatgccgcggctgcccaccctctccgctcagagctcccagccaccaggaaatgcccactttagtgtctacaatcagctctcccagtctcaggtccgagagcgggggcccagcgcctcattcccaagagagcgcggcctcccccaagggtgtgagaggaagccaccctcgccacatctaaatgctgccaatgagtttctctactctcaaaccttctccttgtcccgagaatcttccttacagtaccattcaggaatggggactgtggagaactggactgacgga

SEQ ID NO: 101 - protein, 518 aa, Homo sapiens
MADADEGFGLAHTPLEPDAKDLPCDSKPESALGAPSKSPSSPQAAFTQQGMEGIKVFLHERELWLKFHEVGTEMIITKAGRRMFPSYKVKVTGLNPKTKYILLMDIVPADDHRYKFADNKWSVTGKAEPAMPGRLYVHPDSPATGAHWMRQLVSFQKLKLTNNHLDPFGHIILNSMHKYQPRLHIVKADENNGFGSKNTAFCTHVFPETAFIAVTSYQNHKITQLKIENNPFAKGFRGSDDMELHRMSRMQSKEYPVVPRSTVRQKVASNHSPFSSESRALSTSSNLGSQYQCENGVSGPSQDLLPPPNPYPLPQEHSQIYHCTKRKEEECSTTDHPYKKPYMETSPSEEDSFYRSSYPQQQGLGASYRTESAQRQACMYASSAPPSEPVPSLEDISCNTWPSMPSYSSCTVTTVQPMDRLPYQHFSAHFTSGPLVPRLAGMANHGSPQLGEGMFQHQTSVAHQPVVRQCGPQTGLQSPGTLQPPEFLYSHGVPRTLSPHQYHSVHGVGMVPEWSDNS

SEQ ID NO: 102 - DNA, 1554 bp, Homo sapiens
  source 1..1554: mol_type = genomic DNA
atggccgacgcagacgagggctttggcctggcgcacacgcctctggagcctgacgcaaaagacctgccctgcgattcgaaacccgagagcgcgctcggggcccccagcaagtccccgtcgtccccgcaggccgccttcacccagcagggcatggagggaatcaaagtgtttctccatgaaagagaactgtggctaaaattccacgaagtgggcacggaaatgatcataaccaaggctggaaggcggatgtttcccagttacaaagtgaaggtgacgggccttaatcccaaaacgaagtacattcttctcatggacattgtacctgccgacgatcacagatacaaattcgcagataataaatggtctgtgacgggcaaagctgagcccgccatgcctggccgcctgtacgtgcacccagactcccccgccaccggggcgcattggatgaggcagctcgtctccttccagaaactcaagctcaccaacaaccacctggacccatttgggcatattattctaaattccatgcacaaataccagcctagattacacatcgtgaaagcggatgaaaataatggatttggctcaaaaaatacagcgttctgcactcacgtctttcctgagactgcgtttatagcagtgacttcttaccagaaccacaagatcacgcaattaaagattgagaataatccctttgccaaaggatttcggggcagtgatgacatggagctgcacagaatgtcaagaatgcaaagtaaagaatatcccgtggtccccaggagcaccgtgaggcaaaaagtggcctccaaccacagtcctttcagcagcgagtctcgagctctctccacctcatccaatttggggtcccaataccagtgtgagaatggtgtttccggcccctcccaggacctcctgcctccacccaacccatacccactgccccaggagcatagccaaatttaccattgtaccaagaggaaagaggaagaatgttccaccacagaccatccctataagaagccctacatggagacatcacccagtgaagaagattccttctaccgctctagctatccacagcagcagggcctgggtgcctcctacaggacagagtcggcacagcggcaagcttgcatgtatgccagctctgcgccccccagcgagcctgtgcccagcctagaggacatcagctgcaacacgtggccaagcatgccttcctacagcagctgcaccgtcaccaccgtgcagcccatggacaggctaccctaccagcacttctccgctcacttcacctcggggcccctggtccctcggctggctggcatggccaaccatggctccccacagctgggagagggaatgttccagcaccagacctccgtggcccaccagcctgtggtcaggcagtgtgggcctcagactggcctgcagtcccctggcacccttcagccccctgagttcctctactctcatggcgtgccaaggactctatcccctcatcagtaccactctgtgcacggagttggcatggtgccagagtggagcgacaatagc

SEQ ID NO: 103 - protein, 295 aa, Homo sapiens
MYHPRELYPSLGAGYRLGPAQPGADSSFPPALAEGYRYPELDTPKLDCFLSGMEAAPRTLAAHPPLPLLPPAMGTEPAPSAPEALHSLPGVSLSLENRELWKEFSSVGTEMIITKAGRRMFPACRVSVTGLDPEARYLFLLDVIPVDGARYRWQGRRWEPSGKAEPRLPDRVYIHPDSPATGAHWMRQPVSFHRVKLTNSTLDPHGHLILHSMHKYQPRIHLVRAAQLCSQHWGGMASFRFPETTFISVTAYQNPQITQLKIAANPFAKGFRENGRNCKRWELFIHLFMHSTNVY

SEQ ID NO: 104 - DNA, 885 bp, Homo sapiens
  source 1..885: mol_type = genomic DNA
atgtaccatccacgagaattgtacccgtccctgggggccggctaccgcctggggcccgcccaacctggggccgactccagcttcccacccgccctagcggagggctaccgctaccccgaactggacacccctaaactggattgcttcctctccgggatggaggctgctccccgcaccctggccgcgcacccacctctgccccttctgccccctgccatgggcactgagccggccccatcagctccagaggccctccattccctcccgggggtcagcctgagcctggagaaccgggagctatggaaggagttcagctctgtgggaacagaaatgatcatcaccaaagctgggaggcgcatgttccctgcctgccgagtgtcagtcactggcctggaccccgaggcccgctacttgtttcttctggatgtgattccggtggatggggctcgctaccgctggcagggccggcgctgggagcccagcggcaaggcagagccccgcctgcctgaccgtgtctacattcaccccgactctcctgccactggtgcacattggatgcggcagcctgtgtctttccatcgtgtcaagctcaccaacagcacgctggacccccacggccacctgatcctgcactccatgcacaagtaccaaccccgcatacacctagttcgggcagcccagctctgcagccagcactgggggggcatggcctccttccgcttccccgagaccacattcatctccgtgacagcctaccagaacccacagatcacacaactgaagattgcagccaatccctttgccaaaggcttccgggagaacggcagaaactgtaagaggtgggagttgttcattcatttgttcatgcattcaacaaatgtttat

SEQ ID NO: 105 - protein, 385 aa, Homo sapiens
MAAFLSAGLGILAPSETYPLPTTSSGWEPRLGSPFPSGPCTSSTGAQAVAEPTGQGPKNPRVSRVTVQLEMKPLWEEFNQLGTEMIVTKAGRRMFPPFQVKILGMDSLADYALLMDFIPLDDKRYRYAFHSSAWLVAGKADPATPGRVHFHPDSPAKGAQWMRQIVSFDKLKLTNNLLDDNGHIILNSMHRYQPRFHVVFVDPRKDSERYAQENFKSFIFTETQFTAVTAYQNHRITQLKIASNPFAKGFRESDLDSWPVAPRPLLSVPARSHSSLSPCVLKGATDREKDPNKASASTSKTPAWLHHQLLPPPEVLLAPATYRPVTYQSLYSGAPSHLGIPRTRPAPYPLPNIRADRDQGGLPLPAGLGLLSPTVVCLGPGQDSQ

SEQ ID NO: 106 - DNA, 1155 bp, Homo sapiens
  source 1..1155: mol_type = genomic DNA
atggcagccttcctatctgctggcctcggcatacttgcaccctcagagacctaccccctacctacaaccagctctggctgggagccccggctggggtcaccattcccatcaggcccttgcaccagctctactggggcccaagctgtggccgagcccactgggcagggccccaagaacccacgtgtgtccagagtgacagttcagctggagatgaagcctctgtgggaggaattcaaccagctgggcactgagatgatcgtcaccaaggcaggcaggaggatgttcccccccttccaggtgaagatcctgggcatggactccctggccgactacgccctgctcatggacttcatccccctggacgacaagagatacaggtatgccttccacagctcggcctggctggtggcgggcaaggcagacccagccacacctggccgcgtgcacttccaccccgactcgccagccaagggtgcccagtggatgcgccagattgtgtcctttgacaagctcaagctgaccaacaacctgctggatgacaatggccacatcattctcaactctatgcaccgctaccagccccgtttccacgtggtcttcgtggacccacgcaaggacagtgagcgctatgcccaggagaacttcaagtccttcatcttcacagagacccagttcacagcagtgacagcctatcagaaccacaggatcacccagctgaaaatcgccagcaacccttttgccaaaggctttagagagagtgacctggactcctggcctgtggccccacggcccctgctcagtgtcccagcccggagtcacagcagcctcagtccctgtgtgctgaagggtgccacagacagggagaaagaccccaacaaagcttcagcttccacctccaagacccctgcttggctccatcatcagctgctgcccccacctgaggtcctgctggccccggccacctacaggcctgtcacgtatcagagcctgtactctggagccccgagccacctagggatcccaaggacccgaccagcaccataccccctccccaacatccgggctgatagggatcaaggaggcctgcctctcccagctgggctggggctcctgtcccccactgtggtgtgcctggggcctggccaggactcccag

SEQ ID NO: 107 - protein, 496 aa, Homo sapiens
MSSMEEIQVELQCADLWKRFHDIGTEMIITKAGRRMFPAMRVKITGLDPHQQYYIAMDIVPVDNKRYRYVYHSSKWMVAGNADSPVPPRVYIHPDSLASGDTWMRQVVSFDKLKLTNNELDDQGHIILHSMHKYQPRVHVIRKDFSSDLSPTKPVPVGDGVKTFNFPETVFTTVTAYQNQQITRLKIDRNPFAKGFRDSGRNRTGLEAIMETYAFWRPPVRTLTFEDFTTMQKQQGGSTGTSPTTSSTGTPSPSASSHLLSPSCSPPTFHLAPNTFNVGCRESQLCNLNLSDYPPCARSNMAALQSYPGLSDSGYNRLQSGTTSATQPSETFMPQRTPSLISGIPTPPSLPGNSKMEAYGGQLGSFPTSQFQYVMQAGNAASSSSSPHMFGGSHMQQSSYNAFSLHNPYNLYGYNFPTSPRLAASPEKLSASQSTLLCSSPSNGAFGERQYLPSGMEHSMHMISPSPNNQQATNTCDGRQYGAVPGSSSQMSVHMV

SEQ ID NO: 108 - DNA, 1488 bp, Homo sapiens
  source 1..1488: mol_type = genomic DNA
atgtcttccatggaggagattcaggtggagctgcaatgtgctgacctctggaagcggttccatgatattggaactgaaatgatcatcaccaaagcaggcaggaggatgtttcctgccatgagagtgaaaatcactggcctagatccacatcagcagtactacatagcaatggacattgtgcctgtggacaataaaagatacagatatgtgtatcatagctccaagtggatggtggctggcaatgctgattcccctgtgcccccaagagtttatatacaccctgattctctagcttctggagacacctggatgagacaggtggtcagttttgacaaactcaagcttaccaacaatgagttggatgatcaaggacatatcattctgcactctatgcacaaataccagcctcgagttcatgtgattcgcaaagacttcagcagtgacctttcacccactaagcctgttcctgttggggatggggtgaaaacgttcaactttcctgagactgtgttcaccacagttacggcctatcagaatcagcagattaccagattaaaaattgaccgaaacccttttgctaaaggattcagagattctgggagaaacagaactggacttgaagccatcatggagacatatgcattctggagacctcctgtgcgcacactcaccttcgaagacttcaccaccatgcagaagcagcaaggaggcagcacaggcacttccccaaccacctccagcactgggacaccatccccttcggcttcttctcatcttttatctccatcctgttctcctccaacttttcatctggcccccaacactttcaatgtgggctgccgagaaagccagctgtgtaatctaaacctctctgattatccaccatgtgcccgaagcaacatggctgccttgcagagctacccagggctgagtgacagtggctacaacaggcttcagagtggcaccacttcagccactcagccctctgaaaccttcatgcctcagaggactccatccctgatctcaggaataccaactcctccctcgttgcctggcaacagcaagatggaagcctacggtggccagctggggtcctttcccacttcccagtttcagtatgtcatgcaggcaggcaatgctgcctccagctcctcatcaccacacatgttcgggggcagccacatgcagcagagctcctacaatgccttctcccttcacaacccttacaacctgtatggatacaatttccccacttcccctaggctagctgcaagcccggaaaaactgagcgcctctcaaagcactttactctgttcttctccttccaacggggcctttggagagaggcagtacctgccgtcagggatggagcacagcatgcacatgattagtccttcacccaataaccaacaggcaaccaacacttgtgatggccggcagtatggggcagttccaggctcctcctcccagatgtccgtgcacatggtt

SEQ ID NO: 109 - protein, 607 aa, Homo sapiens [sequence omitted]

SEQ ID NO: 110 - DNA, 1821 bp, Homo sapiens
  source 1..1821: mol_type = genomic DNA
atggccgagaagcgaaggggctcgccgtgcagcatgctaagcctcaaggcgcacgctttctcggtggaggcgctgatcggcgccgagaagcagcaacagcttcagaagaagcggcgaaaactgggcgccgaagaggcggcgagggccgtggacgacggaggctgcagccgcggcggcggcgcgggcgaaaagggttcttctgagggagacgaaggcgctgcgctcccgccgccggctggggcgacgtctgggccggctcggagtggcgcagacctggagcgcggagccgcgggcggctgtgaggacggcttccagcagggagcttcccctctggcgtcaccgggaggctcccccaaggggtctccggcgcgctccctggcccggcccgggacccctctgccctcgccgcaggccccgcgggtggatctgcagggagccgagctctggaagcgctttcatgagataggcactgagatgatcatcaccaaggccggcaggcgcatgtttccagcaatgagagtgaagatctctggattagatcctcaccagcaatattacattgccatggatattgtaccagtggacaacaaaagatacaggtatgtttaccacagttcgaaatggatggtggcaggtaatgctgactcgcctgtgccaccccgtgtgtacattcatccagactcgcctgcctcgggggagacttggatgagacaagttatcagcttcgacaagctgaagctcaccaacaatgaactggatgaccaaggccatattattcttcattctatgcacaaataccaaccgcgagtgcacgtcatccgtaaagactgtggagacgatctttctcccatcaagcctgttccatccggggagggagtaaaggcattctcctttccagaaactgtcttcacaaccgtcactgcctatcagaatcagcagattactcgcctgaagatagataggaatccatttgctaaaggcttccgagactccgggcgcaacagaatgggtttggaagccttggtggaatcatatgcattctggcgaccatcactacggactctgacctttgaagatatccctggaattcccaagcaaggcaatgcaagttcctccaccttgctccaaggtactgggaatggcgttcctgccactcaccctcaccttttgtctggctcctcttgctcctctcctgccttccatctggggcccaacaccagccagctgtgtagtctggcccctgctgactattctgcctgtgcccgctcaggcctcaccctcaaccgatacagcacatctttggcagagacctacaacaggctcaccaaccaggctggtgagacctttgccccgcccaggactccctcctatgtgggcgtgagcagcagcacctccgtgaacatgtccatgggtggcactgatggggacaccttcagctgcccacagaccagcttatccatgcagatttcgggaatgtccccccagctccagtatatcatgccatcaccctccagcaatgccttcgccactaaccagacccatcagggttcctataatacttttagattacacagcccctgtgcactatatggatataacttctccacatcccccaaactggctgccagtcctgagaaaattgtttcttcccaaggaagtttcttggggtcctcaccgagtgggaccatgacggatcggcagatgttgccccctgtggaaggagtgcacctgcttagcagtgggggtcagcagagtttctttgactctaggaccctaggaagcttaactctgtcatcatctcaagtatctgcacatatggtc

SEQ ID NO: 111 - protein, 448 aa, Homo sapiens
MAMSELGTRKPSDGTVSHLLNVVESELQAGREKGDPTEKQLQIILEDAPLWQRFKEVTNEMIVTKNGRRMFPVLKISVTGLDPNAMYSLLLDFVPTDSHRWKYVNGEWVPAGKPEVSSHSCVYIHPDSPNFGAHWMKAPISFSKVKLTNKLNGGGQIMLNSLHKYEPQVHIVRVGSAHRMVTNCSFPETQFIAVTAYQNEEITALKIKYNPFAKAFLDAKERNHLRDVPEAISESQHVTYSHLGGWIFSNPDGVCTAGNSNYQYAAPLPLPAPHTHHGCEHYSGLRGHRQAPYPSAYMHRNHSPSVNLIESSSNNLQVFSGPDSWTSLSSTPHASILSVPHTNGPINPGPSPYPCLWTISNGAGGPSGPGPEVHASTPGAFLLGNPAVTSPPSVLSTQAPTSAGVEVLGEPSLTSIAVSTWTAVASHPFAGWGGPGAGGHHSPSSLDG

SEQ ID NO: 112 - DNA, 1344 bp, Homo sapiens
  source 1..1344: mol_type = genomic DNA
atggccatgagtgagctgggcactcggaagcccagcgatggcactgtttctcatctgctcaatgtggtggagagtgagcttcaggcagggagggaaaaaggcgaccctacggagaagcaacttcagatcatcctggaggatgcacctctctggcagagattcaaggaagtcactaatgagatgattgtgaccaagaatggcagacggatgtttccagtcctaaagattagtgtcacagggttggaccccaatgccatgtactccctcctgctggactttgtccctacggacagtcaccgctggaagtacgtcaacggggaatgggtgcccgctggcaagccagaggtctccagccacagctgcgtctacattcacccggactcccccaactttggggcccactggatgaaagctcccatctccttcagcaaagtgaagctgaccaacaagctcaatggaggcgggcagataatgttgaattctctgcataaatatgaaccccaggttcacatagtgcgtgttggaagtgcccatcgaatggtaacaaactgctccttccctgaaacccagttcatagccgtgactgcctatcagaatgaggagataacggctctcaaaatcaagtacaatccttttgccaaagccttcttggatgccaaggaaagaaatcacctaagagacgtaccggaggctatctctgagagccagcatgtgacctattctcacttgggaggctggatcttttccaatccagatggagtgtgcacagcaggaaactccaattaccagtatgccgctcctctgcctctgcctgctccccacacccaccatggctgtgagcactattcgggtctccgaggacaccggcaggctccctacccttctgcgtacatgcacagaaaccattctccctcagtgaatttgatagaaagctcaagcaataatctgcaagttttctcgggacctgacagctggacttccttatcctccacaccccatgccagcatcctgtctgtaccccacaccaacggaccaatcaatccagggcccagcccctacccgtgcctgtggaccatcagcaatggtgccggaggccccagtgggccaggcccggaggtgcacgccagcaccccaggagcatttctcctcggaaacccagctgtgacttcacccccttctgtgctctccacccaagcacccacttcggctggtgtggaggttctgggggagccctcgctaaccagcattgctgtgtccacctggacagcagtggcctcgcatcccttcgcgggctggggtggcccaggagcgggtgggcaccattctccttcctcactggatggt

SEQ ID NO: 113 - protein, 297 aa, Homo sapiens
MEFTASPKPQLSSRANAFSIAALMSSGGSKEKEATENTIKPLEQFVEKSSCAQPLGELTSLDAHGEFGGGSGSSPSSSSLCTEPLIPTTPIIPSEEMAKIACSLETKELWDKFHELGTEMIITKSGRRMFPTIRVSFSGVDPEAKYIVLMDIVPVDNKRYRYAYHRSSWLVAGKADPPLPARLYVHPDSPFTGEQLLKQMVSFEKVKLTNNELDQHGHIILNSMHKYQPRVHIIKKKDHTASLLNLKSEEFRTFIFPETVFTAVTAYQNQLITKLKIDSNPFAKGFRDSSRLTDIER

SEQ ID NO: 114 - DNA, 892 bp, Homo sapiens
  source 1..892: mol_type = genomic DNA
atggagttcacggcgtcccccaagccccaactctcctcccgggccaacgccttctccattgccgcgctcatgtcgagcggcggctctaaggagaaggaggcgacggagaacacaatcaaacccctggagcaatttgtggagaagtcgtcctgtgcccagcccctgggtgagctgaccagcctggatgctcatggggagtttggtggaggcagtggcagcagcccgtcctcctcctctctgtgcactgagccactgatccccaccacccccatcatccccagtgaggaaatggccaaaattgcctgcagcctggagaccaaggagctttgggacaaattccatgagctgggcaccgagatgatcatcaccaagtcgggcaggaggatgtttccaaccatccgggtgtccttttcgggggtggatcctgaggccaagtacatagtcctgatggacatcgtccctgtggacaacaagaggtaccgctacgcctaccaccggtcctcctggctggtggctggcaaggccgacccgccgttgccagccaggctctatgtgcatccagattctccttttaccggtgagcaactactcaaacagatggtgtcttttgaaaaggtgaaactcaccaacaatgaactggatcaacatggccatataattttgaactcaatgcataagtaccagccaagggtgcacatcattaagaagaaagaccacacagcctcattgctcaacctgaagtctgaagaatttagaactttcatctttccagaaacagtttttacggcagtcactgcctaccagaatcaactgataacgaagctgaaaatagatagcaatccttttgccaaaggattccgggattcctccaggctcactgacattgagaggt

SEQ ID NO: 115 - protein, 535 aa, Homo sapiens
MGIVEPGCGDMLTGTEPMPGSDEGRAPGADPQHRYFYPEPGAQDADERRGGGSLGSPYPGGALVPAPPSRFLGAYAYPPRPQAAGFPGAGESFPPPADAEGYQPGEGYAAPDPRAGLYPGPREDYALPAGLEVSGKLRVALNNHLLWSKFNQHQTEMIITKQGRRMFPFLSFTVAGLEPTSHYRMFVDVVLVDQHHWRYQSGKWVQCGKAEGSMPGNRLYVHPDSPNTGAHWMRQEVSFGKLKLTNNKGASNNVTQMIVLQSLHKYQPRLHIVEVNDGEPEAACNASNTHIFTFQETQFIAVTAYQNAEITQLKIDNNPFAKGFRENFESMYTSVDTSIPSPPGPNCQFLGGDHYSPLLPNQYPVPSRFYPDLPGQAKDVVPQAYWLGAPRDHSYEAEFRAVSMKPAFLPSAPGPTMSYYRGQEVLAPGAGWPVAPQYPPKMGPASWFRPMRTLPMEPGPGGSEGRGPEDQGPPLVWTEIAPIRPESSDSGLGEGDSKRRRVSPYPSSGDSSSPAGAPSPFDKEAEGQFYNYFPN

SEQ ID NO: 116 - DNA, 1605 bp, Homo sapiens
  source 1..1605: mol_type = genomic DNA
atgggcatcgtggagccgggttgcggagacatgctgacgggcaccgagccgatgccggggagcgacgagggccgggcgcctggcgccgacccgcagcaccgctacttctacccggagccgggcgcgcaggacgcggacgagcgtcgcgggggcggcagcctggggtctccctacccggggggcgccttggtgcccgccccgccgagccgcttccttggagcctacgcctacccgccgcgaccccaggcggccggcttccccggcgcgggcgagtccttcccgccgcccgcggacgccgagggctaccagccgggcgagggctacgccgccccggacccgcgcgccgggctctacccggggccgcgtgaggactacgcgctacccgcgggactggaggtgtcggggaaactgagggtcgcgctcaacaaccacctgttgtggtccaagtttaatcagcaccagacagagatgatcatcaccaagcagggacggcggatgttcccattcctgtcatttactgtggccgggctggagcccaccagccactacaggatgtttgtggacgtggtcttggtggaccagcaccactggcggtaccagagcggcaagtgggtgcagtgtggaaaggccgagggcagcatgccaggaaaccgcctgtacgtccacccggactcccccaacacaggagcgcactggatgcgccaggaagtttcatttgggaaactaaagctcacaaacaacaagggggcgtccaacaatgtgacccagatgattgtgctccagtccctccataagtaccagccccggctgcatatcgttgaggtgaacgacggagagccagaggcagcctgcaacgcttccaacacgcatatctttactttccaagaaacccagttcattgccgtgactgcctaccagaatgccgagattactcagctgaaaattgataataacccctttgccaaaggattccgggagaactttgagtccatgtacacatctgttgacaccagcatcccctccccgcctggacccaactgtcaattccttgggggagatcactactctcctctcctacccaaccagtatcctgttcccagccgcttctaccccgaccttcctggccaggcgaaggatgtggttccccaggcttactggctgggggccccccgggaccacagctatgaggctgagtttcgagcagtcagcatgaagcctgcattcttgccctctgcccctgggcccaccatgtcctactaccgaggccaggaggtcctggcacctggagctggctggcctgtggcaccccagtaccctcccaagatgggcccggccagctggttccgccctatgcggactctgcccatggaacccggccctggaggctcagagggacggggaccagaggaccagggtccccccttggtgtggactgagattgcccccatccggccggaatccagtgattcaggactgggcgaaggagactctaagaggaggcgcgtgtccccctatccttccagtggtgacagctcctcccctgctggggccccttctccttttgataaggaagctgaaggacagttttataactattttcccaac

SEQ ID NO: 117 - protein, 520 aa, Homo sapiens
MALSSRARAFSVEALVGRPSKRKLQDPIQAEQPELREKKGGEEEEERRSSAAGKSEPLEKQPKTEPSTSASSGCGSDSGYGNSSESLEEKDIQMELQGSELWKRFHDIGTEMIITKAGRRMFPSVRVKVKGLDPGKQYHVAIDVVPVDSKRYRYVYHSSQWMVAGNTDHLCIIPRFYVHPDSPCSGETWMRQIISFDRMKLTNNEMDDKGHIILQSMHKYKPRVHVIEQGSSVDLSQIQSLPTEGVKTFSFKETEFTTVTAYQNQQITKLKIERNPFAKGFRDTGRNRGVLDGLLETYPWRPSFTLDFKTFGADTQSGSSGSSPVTSSGGAPSPLNSLLSPLCFSPMFHLPTSSLGMPCPEAYLPNVNLPLCYKICPTNFWQQQPLVLPAPERLASSNSSQSLAPLMMEVPMLSSLGVTNSKSGSSEDSSDQYLQAPNSTNQMLYGLQSPGNIFLPNSITPEALSCSFHPSYDFYRYNFSMPSRLISGSNHLKVNDDSQVSFGEGKCNHVHWYPAINHYL

SEQ ID NO: 118 - DNA, 1560 bp, Homo sapiens
  source 1..1560: mol_type = genomic DNA
atggctctgagctctcgggcgcgtgccttctccgtggaagccttggtggggagacccagcaaaagaaaactccaagacccaatacaggcggagcagcctgagctgcgggagaaaaagggcggagaggaagaggaggagagaaggagcagcgctgcagggaagagcgagccgcttgaaaaacaacctaagacagagccctcaacatctgcttcctctggctgcggcagcgacagcggctacggcaacagctctgaaagtctggaagagaaagatattcaaatggagcttcaaggatctgaactgtggaaaagattccatgacatcgggactgagatgatcattactaaagcgggcaggcggatgttcccctctgttcgggtcaaggtgaaagggttggatccagggaagcagtaccatgtggccatcgatgtggtgccggtggattccaaacgctataggtacgtctatcacagctcacagtggatggtagctgggaatacagaccatttgtgcatcattcctagattctatgttcacccggactcaccctgctcgggagagacctggatgcggcagatcatcagctttgatcgcatgaaactcaccaacaatgagatggatgacaaaggccacatcattctgcaatccatgcataagtacaaaccccgagtgcacgtgatagagcaaggcagcagtgttgacctgtcccagattcagtccttgcccactgaaggtgttaaaacattctcctttaaagaaactgagttcaccacagtaacggcttaccaaaaccaacagattacgaaactaaaaatagaaagaaatccttttgctaaaggatttagagatactggaagaaacaggggtgtattggatgggcttttagagacctacccatggaggccttctttcactctcgattttaaaacctttggcgcagacacacaaagtggaagcagtggctcatctccagtgacctctagtggaggggccccctctcctttgaactccttactttctccactttgcttttcacctatgtttcatttacctacaagctcccttggaatgccctgtccagaggcatacctgcccaatgtcaacctgcctctatgctacaagatttgtccaactaatttttggcaacagcaacctcttgttttaccggctcctgaaagactagcaagcagcaacagttctcagtctttagccccactcatgatggaagtgcctatgttatcttccctgggggtcaccaattcaaaaagcggttcatctgaagactccagtgatcagtatctacaagcacctaattctaccaatcaaatgttatatggattacagtcacctggaaatatttttctgccaaactccatcaccccagaagcacttagttgctcctttcatccttcctatgacttttatagatacaatttctctatgccatctagactgataagtggttccaaccatcttaaagtgaatgacgacagtcaagtttcttttggagaaggcaaatgtaatcatgttcattggtatccagcaattaaccattacctt

SEQ ID NO: 119 - protein, 440 aa, Homo sapiens
MEQTYGEVNQLGGVFVNGRPLPNAIRLRIVELAQLGIRPCDISRQLRVSHGCVSKILARYNETGSILPGAIGGSKPRVTTPNVVKHIRDYKQGDPGIFAWEIRDRLLADGVCDKYNVPSVSSISRILRNKIGSLAQPGPYEASKQPPSQPTLPYNHIYQYPYPSPVSPTGAKMGSHPGVPGTAGHVSIPRSWPSAHSVSNILGIRTFMEQTGALAGSEGTAYSPKMEDWAGVNRTAFPATPAVNGLEKPALEADIKYTQSASTLSAVGGFLPACAYPASNQHGVYSAPGGGYLAPGPPWPPAQGPPLAPPGAGVAVHGGELAAAMTFKHPSREGSLPAPAARPRTPSVAYTDCPSRPRPPRGSSPRTRARRERQADPGAQVCAAAPAIGTGRIGGLAEEEASAGPRGARPASPQAQPCLWPDPPHFLYWSGFLGFSELGF

SEQ ID NO: 120 - DNA, 1320 bp, Homo sapiens
  source 1..1320: mol_type = genomic DNA
atggagcagacgtatggcgaggtgaaccagctgggcggtgtgttcgtcaacggccgccccctgcccaacgccatccgcttgcgcattgtggagctggcgcagctgggcatccgaccctgtgacatcagtcggcagctccgcgtatcccacggctgcgtgagcaagatcctggcgcgctacaacgagaccggctccattctgcccggggccatcggggggagcaagccccgcgtcaccactcccaacgtggtcaagcacatccgggactacaagcaaggagaccctggcatctttgcctgggagatccgcgaccggctgctggccgacggcgtctgtgacaagtacaatgtgccttcggtgagctccatcagccgcatcctgcgcaacaagatcggcagcctggcgcagcccggaccgtacgaggcaagtaagcagccgccgtcgcagcctacgctgccctacaaccacatctaccagtacccctaccccagtcccgtgtcgcccacgggcgccaagatgggcagccaccccggggtcccgggcacggcgggccacgtcagcatcccgcgctcatggccctcggcacactcggtcagcaacatcctgggcatccggacgtttatggagcaaacaggggccctggctgggagcgaaggcaccgcttactctcccaagatggaagactgggccggcgtgaaccgcacggccttccccgccacccccgcagtgaatgggctagagaaacctgccttagaggcagacattaaatacactcagtcggcctccaccctctctgccgtgggcggctttctccccgcctgcgcctacccggcctccaaccagcacggcgtgtacagcgccccgggcggcggctacctcgccccgggcccgccgtggccgcctgcgcaaggtcctcctctggcgccccccggggccggcgtagctgtgcatggcggggaactcgcggcagcaatgaccttcaagcatcccagccgagaaggaagcctcccagctccggcagcaaggccccggacgccctcagtagcttacacggactgcccatcccggcctcgacctcctaggggcagctctccccggacccgagcccggagggaacggcaggcggacccgggcgcacaggtctgcgcggcggccccggcaatcggcacgggcaggatcggaggactcgcggaggaggaagccagtgccggcccgcggggtgcacgcccagccagcccccaggcccagccctgcctctggccggacccaccacacttcctttattggtctgggtttttaggcttctctgaacttgggttt

SEQ ID NO: 121 - protein, 204 aa, Homo sapiens
MQSYASAMLSVFNSDDYSPAVQENIPALRRSSSFLCTESCNSKYQCETGENSKGNVQDRVKRPMNAFIVWSRDQRRKMALENPRMRNSEISKQLGYQWKMLTEAEKWPFFQEAQKLQAMHREKYPNYKYRPRRKAKMLPKNCSLLPADPASVLCSEVQLDNRLYRDDCTKATHSRMEHQLGHLPPINAASSPQQRDRYSHWTKL

SEQ ID NO: 122 - DNA, 612 bp, Homo sapiens
  source 1..612: mol_type = genomic DNA
atgcaatcatatgcttctgctatgttaagcgtattcaacagcgatgattacagtccagctgtgcaagagaatattcccgctctccggagaagctcttccttcctttgcactgaaagctgtaactctaagtatcagtgtgaaacgggagaaaacagtaaaggcaacgtccaggatagagtgaagcgacccatgaacgcattcatcgtgtggtctcgcgatcagaggcgcaagatggctctagagaatcccagaatgcgaaactcagagatcagcaagcagctgggataccagtggaaaatgcttactgaagccgaaaaatggccattcttccaggaggcacagaaattacaggccatgcacagagagaaatacccgaattataagtatcgacctcgtcggaaggcgaagatgctgccgaagaattgcagtttgcttcccgcagatcccgcttcggtactctgcagcgaagtgcaactggacaacaggttgtacagggatgactgtacgaaagccacacactcaagaatggagcaccagctaggccacttaccgcccatcaacgcagccagctcaccgcagcaacgggaccgctacagccactggacaaagctg

SEQ ID NO: 123 - protein, 391 aa, Homo sapiens
MYSMMMETDLHSPGGAQAPTNLSGPAGAGGGGGGGGGGGGGGGAKANQDRVKRPMNAFMVWSRGQRRKMAQENPKMHNSEISKRLGAEWKVMSEAEKRPFIDEAKRLRALHMKEHPDYKYRPRRKTKTLLKKDKYSLAGGLLAAGAGGGGAAVAMGVGVGVGAAAVGQRLESPGGAAGGGYAHVNGWANGAYPGSVAAAAAAAAMMQEAQLAYGQHPGAGGAHPHAHPAHPHPHHPHAHPHNPQPMHRYDMGALQYSPISNSQGYMSASPSGYGGLPYGAAAAAAAAAGGAHQNSAVAAAAAAAAASSGALGALGSLVKSEPSGSPPAPAHSRAPCPGDLREMISMYLPAGEGGDPAAAAAAAAQSRLHSLPQHYQGAGAGVNGTVPLTHI

SEQ ID NO: 124 - DNA, 1173 bp, Homo sapiens
  source 1..1173: mol_type = genomic DNA
atgtacagcatgatgatggagaccgacctgcactcgcccggcggcgcccaggcccccacgaacctctcgggccccgccggggcgggcggcggcgggggcggaggcgggggcggcggcggcggcgggggcgccaaggccaaccaggaccgggtcaaacggcccatgaacgccttcatggtgtggtcccgcgggcagcggcgcaagatggcccaggagaaccccaagatgcacaactcggagatcagcaagcgcctgggggccgagtggaaggtcatgtccgaggccgagaagcggccgttcatcgacgaggccaagcggctgcgcgcgctgcacatgaaggagcacccggattacaagtaccggccgcgccgcaagaccaagacgctgctcaagaaggacaagtactcgctggccggcgggctcctggcggccggcgcgggtggcggcggcgcggctgtggccatgggcgtgggcgtgggcgtgggcgcggcggccgtgggccagcgcctggagagcccaggcggcgcggcgggcggcggctacgcgcacgtcaacggctgggccaacggcgcctaccccggctcggtggcggcggcggcggcggccgcggccatgatgcaggaggcgcagctggcctacgggcagcacccgggcgcgggcggcgcgcacccgcacgcgcaccccgcgcacccgcacccgcaccacccgcacgcgcacccgcacaacccgcagcccatgcaccgctacgacatgggcgcgctgcagtacagccccatctccaactcgcagggctacatgagcgcgtcgccctcgggctacggcggcctcccctacggcgccgcggccgccgccgccgccgctgcgggcggcgcgcaccagaactcggccgtggcggcggcggcggcggcggcggccgcgtcgtcgggcgccctgggcgcgctgggctctctggtgaagtcggagcccagcggcagcccgcccgccccagcgcactcgcgggcgccgtgccccggggacctgcgcgagatgatcagcatgtacttgcccgccggcgaggggggcgacccggcggcggcagcagcggccgcggcgcagagccggctgcactcgctgccgcagcactaccagggcgcgggcgcgggcgtgaacggcacggtgcccctgacgcacatc

SEQ ID NO: 125 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 126 - DNA, 951 bp, Homo sapiens
  source 1..951: mol_type = genomic DNA
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatg

SEQ ID NO: 127 - protein, 446 aa, Homo sapiens
MRPVRENSSGARSPRVPADLARSILISLPFPPDSLAHRPPSSAPTESQGLFTVAAPAPGAPSPPATLAHLLPAPAMYSLLETELKNPVGTPTQAAGTGGPAAPGGAGKSSANAAGGANSGGGSSGGASGGGGGTDQDRVKRPMNAFMVWSRGQRRKMALENPKMHNSEISKRLGADWKLLTDAEKRPFIDEAKRLRAVHMKEYPDYKYRPRRKTKTLLKKDKYSLPSGLLPPGAAAAAAAAAAAAAAASSPVGVGQRLDTYTHVNGWANGAYSLVQEQLGYAQPPSMSSPPPPPALPPMHRYDMAGLQYSPMMPPGAQSYMNVAAAAAAASGYGGMAPSATAAAAAAYGQQPATAAAAAAAAAAMSLGPMGSVVKSEPSSPPPAIASHSQRACLGDLRDMISMYLPPGGDAADAASPLPGGRLHGVHQHYQGAGTAVNGTVPLTHI

SEQ ID NO: 128 - DNA, 1338 bp, Homo sapiens
  source 1..1338: mol_type = genomic DNA
atgcgacctgttcgagagaactcatcaggtgcgagaagcccgcgggttcctgctgatttggcgcggagcattttgataagcctacccttcccgccggactcgctggcccacaggcccccaagctccgctccgacggagtcccagggccttttcaccgtggccgctccagccccgggagcgccttctcctcccgccacgctggcgcaccttcttcccgccccggcaatgtacagccttctggagactgaactcaagaaccccgtagggacacccacacaagcggcgggcaccggcggccccgcagccccgggaggcgcaggcaagagtagtgcgaacgcagccggcggcgcgaactcgggcggcggcagcagcggtggtgcgagcggaggtggcgggggtacagaccaggaccgtgtgaaacggcccatgaacgccttcatggtatggtcccgcgggcagcggcgcaaaatggccctggagaaccccaagatgcacaattctgagatcagcaagcgcttgggcgccgactggaaactgctgaccgacgccgagaagcgaccattcatcgacgaggccaagcgacttcgcgccgtgcacatgaaggagtatccggactacaagtaccgaccgcgccgcaagaccaagacgctgctcaagaaagataagtactccctgcccagcggcctcctgcctcccggtgccgcggccgccgccgccgctgccgcggccgcagccgctgccgccagcagtccggtgggcgtgggccagcgcctggacacgtacacgcacgtgaacggctgggccaacggcgcgtactcgctggtgcaggagcagctgggctacgcgcagcccccgagcatgagcagcccgccgccgccgcccgcgctgccgccgatgcaccgctacgacatggccggcctgcagtacagcccaatgatgccgcccggcgctcagagctacatgaacgtcgctgccgcggccgccgccgcctcgggctacgggggcatggcgccctcagccacagcagccgcggccgccgcctacgggcagcagcccgccaccgccgcggccgcagctgcggccgcagccgccatgagcctgggccccatgggctcggtagtgaagtctgagcccagctcgccgccgcccgccatcgcatcgcactctcagcgcgcgtgcctcggcgacctgcgcgacatgatcagcatgtacctgccacccggcggggacgcggccgacgccgcctctccgctgcccggcggtcgcctgcacggcgtgcaccagcactaccagggcgccgggactgcagtcaacggaacggtgccgctgacccacatc

SEQ ID NO: 129 - protein, 240 aa, Homo sapiens
MSKPSDHIKRPMNAFMVWSRGQRRKMAQENPKMHNSEISKRLGAEWKLLSEAEKRPYIDEAKRLRAQHMKEHPDYKYRPRRKPKNLLKKDRYVFPLPYLGDTDPLKAAGLPVGASDGLLSAPEKARAFLPPASAPYSLLDPAQFSSSAIQKMGEVPHTLATGALPYASTLGYQNGAFGSLSCPSQHTHTHPSPTNPGYVVPCNCTAWSASTLQPPVAYILFPGMTKTGIDPYSSAHATAM

SEQ ID NO: 130 - DNA, 720 bp, Homo sapiens
  source 1..720: mol_type = genomic DNA
atgtccaaaccttcagaccacatcaagcggcccatgaacgccttcatggtatggtcccggggccagcggcgcaagatggcccaggaaaaccccaagatgcacaactcggagatcagcaaacgcctaggtgccgaatggaagcttctgtccgaggcagagaagcggccatacatcgatgaagccaagcggctacgcgcccagcacatgaaggagcaccctgactacaagtaccgacctcggcgcaagcccaagaacctgctcaagaaggacaggtatgtcttccccttgccctacctgggcgacacggacccgctcaaggcggctggcctgcccgtgggggcctccgacggcctcctgagcgcgcccgagaaagcccgggccttcttgccgccggcctcggcgccctactccctgctggaccccgcgcagtttagctcgagcgccatccagaagatgggcgaagtgccccacaccttggctaccggcgctctgccctacgcgtccaccctgggctaccagaacggcgccttcggcagcctcagctgccccagccagcacacgcacacgcacccgtcccccaccaaccctggctacgtggtgccctgtaactgtaccgcctggtctgcctccaccctgcagccccccgtcgcctacatcctcttcccaggcatgaccaagactggcatagacccttattcgtcagcccacgctacggccatg

SEQ ID NO: 131 - protein, 276 aa, Homo sapiens
MSKPVDHVKRPMNAFMVWSRAQRRKMAQENPKMHNSEISKRLGAEWKLLTESEKRPFIDEAKRLRAMHMKEHPDYKYRPRRKPKTLLKKDKFAFPVPYGLGGVADAEHPALKAGAGLHAGAGGGLVPESLLANPEKAAAAAAAAAARVFFPQSAAAAAAAAAAAAAGSPYSLLDLGSKMAEISSSSSGLPYASSLGYPTAGAGAFHGAAAAAAAAAAAAGGHTHSHPSPGNPGYMIPCNCSAWPSPGLQPPLAYILLPGMGKPQLDPYPAAYAAAL

SEQ ID NO: 132 - DNA, 828 bp, Homo sapiens
  source 1..828: mol_type = genomic DNA
atgtccaagccggtggaccacgtcaagcggcccatgaacgccttcatggtgtggtcgcgggctcagcggcgcaagatggcccaggagaaccccaagatgcacaactcggagatcagcaagcgcttgggcgccgagtggaaactgctcacagagtcggagaagcggccgttcatcgacgaggccaagcgtctacgcgccatgcacatgaaggagcaccccgactacaagtaccggccgcggcgcaagcccaagacgctgctcaagaaggacaagttcgccttcccggtgccctacggcctgggcggcgtggcggacgccgagcaccctgcgctcaaggcgggcgccgggctgcacgcgggggcgggcggcggcctggtgcctgagtcgctgctcgccaatcccgagaaggcggccgcggccgccgccgctgccgccgcacgcgtcttcttcccgcagtcggccgctgccgccgccgctgccgccgccgccgccgccgcgggcagcccctactcgctgctcgacctgggctccaaaatggcagagatctcgtcgtcctcgtccggcctcccgtacgcgtcgtcgctgggctacccgaccgcgggcgcgggcgccttccacggcgcggcggcggcggctgcagcggcggccgccgccgccggggggcacacgcactcgcaccccagcccgggcaacccgggctacatgatcccgtgcaactgcagcgcgtggcccagccccgggctgcagccgccgctcgcctacatcctgctgccgggcatgggcaagccccagctggacccctaccccgcggcctacgctgccgcgcta

SEQ ID NO: 133 - protein, 474 aa, Homo sapiens
MVQQTNNAENTEALLAGESSDSGAGLELGIASSPTPGSTASTGGKADDPSWCKTPSGHIKRPMNAFMVWSQIERRKIMEQSPDMHNAEISKRLGKRWKLLKDSDKIPFIREAERLRLKHMADYPDYKYRPRKKVKSGNANSSSSAAASSKPGEKGDKVGGSGGGGHGGGGGGGSSNAGGGGGGASGGGANSKPAQKKSCGSKVAGGAGGGVSKPHAKLILAGGGGGGKAAAAAAASFAAEQAGAAALLPLGAAADHHSLYKARTPSASASASSAASASAALAAPGKHLAEKKVKRVYLFGGLGTSSSPVGGVGAGADPSDPLGLYEEEGAGCSPDAPSLSGRSSAASSPAAGRSPADHRGYASLRAASPAPSSAPSHASSSASSHSSSSSSSGSSSSDDEFEDDLLDLNPSSNFESMSLGSFSSSSALDRDLDFNFEPGSGSHFEFPDYCTPEVSEMISGDWLESSISNLVFTY

SEQ ID NO: 134 - DNA, 1422 bp, Homo sapiens
  source 1..1422: mol_type = genomic DNA
atggtgcagcaaaccaacaatgccgagaacacggaagcgctgctggccggcgagagctcggactcgggcgccggcctcgagctgggaatcgcctcctcccccacgcccggctccaccgcctccacgggcggcaaggccgacgacccgagctggtgcaagaccccgagtgggcacatcaagcgacccatgaacgccttcatggtgtggtcgcagatcgagcggcgcaagatcatggagcagtcgcccgacatgcacaacgccgagatctccaagcggctgggcaaacgctggaagctgctcaaagacagcgacaagatccctttcattcgagaggcggagcggctgcgcctcaagcacatggctgactaccccgactacaagtaccggcccaggaagaaggtgaagtccggcaacgccaactccagctcctcggccgccgcctcctccaagccgggggagaagggagacaaggtcggtggcagtggcgggggcggccatgggggcggcggcggcggcgggagcagcaacgcggggggaggaggcggcggtgcgagtggcggcggcgccaactccaaaccggcgcagaaaaagagctgcggctccaaagtggcgggcggcgcgggcggtggggttagcaaaccgcacgccaagctcatcctggcaggcggcggcggcggcgggaaagcagcggctgccgccgccgcctccttcgccgccgaacaggcgggggccgccgccctgctgcccctgggcgccgccgccgaccaccactcgctgtacaaggcgcggactcccagcgcctcggcctccgcctcctcggcagcctcggcctccgcagcgctcgcggccccgggcaagcacctggcggagaagaaggtgaagcgcgtctacctgttcggcggcctgggcacgtcgtcgtcgcccgtgggcggcgtgggcgcgggagccgaccccagcgaccccctgggcctgtacgaggaggagggcgcgggctgctcgcccgacgcgcccagcctgagcggccgcagcagcgccgcctcgtcccccgccgccggccgctcgcccgccgaccaccgcggctacgccagcctgcgcgccgcctcgcccgccccgtccagcgcgccctcgcacgcgtcctcctcggcctcgtcccactcctcctcttcctcctcctcgggctcctcgtcctccgacgacgagttcgaagacgacctgctcgacctgaaccccagctcaaactttgagagcatgtccctgggcagcttcagttcgtcgtcggcgctcgaccgggacctggattttaacttcgagcccggctccggctcgcacttcgagttcccggactactgcacgcccgaggtgagcgagatgatctcgggagactggctcgagtccagcatctccaacctggttttcacctac

SEQ ID NO: 135 - protein, 441 aa, Homo sapiens
MVQQAESLEAESNLPREALDTEEGEFMACSPVALDESDPDWCKTASGHIKRPMNAFMVWSKIERRKIMEQSPDMHNAEISKRLGKRWKMLKDSEKIPFIREAERLRLKHMADYPDYKYRPRKKPKMDPSAKPSASQSPEKSAAGGGGGSAGGGAGGAKTSKGSSKKCGKLKAPAAAGAKAGAGKAAQSGDYGGAGDDYVLGSLRVSGSGGGGAGKTVKCVFLDEDDDDDDDDDELQLQIKQEPDEEDEEPPHQQLLQPPGQQPSQLLRRYNVAKVPASPTLSSSAESPEGASLYDEVRAGATSGAGGGSRLYYSFKNITKQHPPPLAQPALSPASSRSVSTSSSSSSGSSSGSSGEDADDLMFDLSLNFSQSAHSASEQQLGGGAAAGNLSLSLVDKDLDSFSEGSLGSHFEFPDYCTPELSEMIAGDWLEANFSDLVFTY

SEQ ID NO: 136 - DNA, 1323 bp, Homo sapiens
  source 1..1323: mol_type = genomic DNA
atggtgcagcaggcggagagcttggaagcggagagcaacctgccccgggaggcgctggacacggaggagggcgaattcatggcttgcagcccggtggccctggacgagagcgacccagactggtgcaagacggcgtcgggccacatcaagcggccgatgaacgcgttcatggtatggtccaagatcgaacgcaggaagatcatggagcagtctccggacatgcacaacgccgagatctccaagaggctgggcaagcgctggaaaatgctgaaggacagcgagaagatcccgttcatccgggaggcggagcggctgcggctcaagcacatggccgactaccccgactacaagtaccggccccggaaaaagcccaaaatggacccctcggccaagcccagcgccagccagagcccagagaagagcgcggccggcggcggcggcgggagcgcgggcggaggcgcgggcggtgccaagacctccaagggctccagcaagaaatgcggcaagctcaaggcccccgcggccgcgggcgccaaggcgggcgcgggcaaggcggcccagtccggggactacgggggcgcgggcgacgactacgtgctgggcagcctgcgcgtgagcggctcgggcggcggcggcgcgggcaagacggtcaagtgcgtgtttctggatgaggacgacgacgacgacgacgacgacgacgagctgcagctgcagatcaaacaggagccggacgaggaggacgaggaaccaccgcaccagcagctcctgcagccgccggggcagcagccgtcgcagctgctgagacgctacaacgtcgccaaagtgcccgccagccctacgctgagcagctcggcggagtcccccgagggagcgagcctctacgacgaggtgcgggccggcgcgacctcgggcgccgggggcggcagccgcctctactacagcttcaagaacatcaccaagcagcacccgccgccgctcgcgcagcccgcgctgtcgcccgcgtcctcgcgctcggtgtccacctcctcgtccagcagcagcggcagcagcagcggcagcagcggcgaggacgccgacgacctgatgttcgacctgagcttgaatttctctcaaagcgcgcacagcgccagcgagcagcagctggggggcggcgcggcggccgggaacctgtccctgtcgctggtggataaggatttggattcgttcagcgagggcagcctgggctcccacttcgagttccccgactactgcacgccggagctgagcgagatgatcgcgggggactggctggaggcgaacttctccgacctggtgttcacatat

SEQ ID NO: 137 - protein, 315 aa, Homo sapiens
MVQQRGARAKRDGGPPPPGPGPAEEGAREPGWCKTPSGHIKRPMNAFMVWSQHERRKIMDQWPDMHNAEISKRLGRRWQLLQDSEKIPFVREAERLRLKHMADYPDYKYRPRKKSKGAPAKARPRPPGGSGGGSRLKPGPQLPGRGGRRAAGGPLGGGAAAPEDDDEDDDEELLEVRLVETPGRELWRMVPAGRAARGQAERAQGPSGEGAAAAAAASPTPSEDEEPEEEEEEAAAAEEGEEETVASGEESLGFLSRLPPGPAGLDCSALDRDPDLQPPSGTSHFEFPDYCTPEVTEMIAGDWRPSSIADLVFTY

SEQ ID NO: 138 - DNA, 945 bp, Homo sapiens
  source 1..945: mol_type = genomic DNA
atggtgcagcagcggggcgcgagggccaagcgggacggcgggccgccgcccccgggacccgggccggccgaggagggggcgcgcgagcccggctggtgcaagaccccgagcggccacatcaagaggccgatgaacgcattcatggtgtggtcgcagcacgaacggcggaagatcatggaccagtggcccgacatgcacaacgccgagatctccaagcgcctgggccgccgctggcagctgctgcaggactcggagaagatcccgttcgtgcgggaggcggagcggctgcggctcaagcacatggcggattacccggactacaagtaccggccgcgcaaaaagagcaagggggcgcccgccaaggcgcggccccgcccccccggtggtagcggtggcggcagccggctcaagcccgggccgcagctgcctggccgcgggggccgccgagcagcgggagggcctttggggggcggggcggcggcgcccgaggacgacgatgaagacgacgacgaggagctgctggaagtgcgcctggtcgagaccccggggcgggagctgtggaggatggtcccggcgggacgggccgctcggggacaagcggagcgcgcccaagggccgtcgggcgagggggcggccgccgccgccgccgcctccccgacaccgtcggaggacgaggagccggaggaagaggaggaggaggcggcagcggctgaggaaggtgaagaggagacggtggcgtcgggggaggagtcgctgggctttctgtccaggctgccccctggcccggccggcctggactgcagcgccctggatcgcgacccggacctgcagcctccctcgggcacgtcgcacttcgagttcccggactactgcacccccgaggttaccgagatgatcgcgggggactggcgcccgtctagcatcgcagacctggttttcacctac

SEQ ID NO: 139 - protein, 763 aa, Homo sapiens [sequence omitted]

SEQ ID NO: 140 - DNA, 2289 bp, Homo sapiens
  source 1..2289: mol_type = genomic DNA
atgcttactgaccctgatttacctcaggagtttgaaaggatgtcttccaagcgaccagcctctccgtatggggaagcagatggagaggtagccatggtgacaagcagacagaaagtggaagaagaggagagtgacgggctcccagcctttcaccttcccttgcatgtgagttttcccaacaagcctcactctgaggaatttcagccagtttctctgctgacgcaagagacttgtggccataggactcccacttctcagcacaatacaatggaagttgatggcaataaagttatgtcttcatttgccccacacaactcatctacctcacctcagaaggcagaagaaggtgggcgacagagtggcgagtccttgtctagtacagccctgggaactcctgaacggcgcaagggcagtttagctgatgttgttgacaccttgaagcagaggaaaatggaagagctcatcaaaaacgagccggaagaaacccccagtattgaaaaactactctcaaaggactggaaagacaagcttcttgcaatgggatcggggaactttggcgaaataaaagggactcccgagagcttagctgagaaagaaaggcaactcatgggtatgatcaaccagctgaccagcctccgagagcagctgttggctgcccacgatgagcagaagaaactagctgcctctcagattgagaaacagcgtcagcaaatggagctggccaagcagcaacaagaacaaattgcaagacagcagcagcagcttctacagcaacaacacaaaatcaatttgctccagcaacagatccaggttcaaggtcagctgccgccattaatgattcccgtattccctcctgatcaacggacactggctgcagctgcccagcaaggattcctcctccctccaggcttcagctataaggctggatgtagtgacccttaccctgttcagctgatcccaactaccatggcagctgctgccgcagcaacaccaggcttaggcccactccaactgcagcagttatatgctgcccagctagctgcaatgcaggtatctccaggagggaagctgccaggcataccccaaggcaaccttggtgctgctgtatctcctaccagcattcacacagacaagagcacaaacagcccaccacccaaaagcaaggatgaagtggcacagccactgaacctatcagctaaacccaagacctctgatggcaaatcacccacatcacccacctctccccatatgccagctctgagaataaacagtggggcaggccccctcaaagcctctgtcccagcagcgttagctagtccttcagccagagttagcacaataggttacttaaatgaccatgatgctgtcaccaaggcaatccaagaagctcggcaaatgaaggagcaactccgacgggaacaacaggtgcttgatgggaaggtggctgttgtgaatagtctgggtctcaataactgccgaacagaaaaggaaaaaacaacactggagagtctgactcagcaactggcagttaaacagaatgaagaaggaaaatttagccatgcaatgatggatttcaatctgagtggagattctgatggaagtgctggagtctcagagtcaagaatttatagggaatcccgagggcgtggtagcaatgaaccccacataaagcgtccaatgaatgccttcatggtgtgggctaaagatgaacggagaaagatccttcaagcctttcctgacatgcacaactccaacatcagcaagatattgggatctcgctggaaagctatgacaaacctagagaaacagccatattatgaggagcaagcccgtctcagcaagcagcacctggagaagtaccctgactataagtacaagcccaggccaaagcgcacctgcctggtggatggcaaaaagctgcgcattggtgaatacaaggcaatcatgcgcaacaggcggcaggaaatgcggcagtacttcaatgttgggcaacaagcacagatccccattgccactgctggtgttgtgtaccctggagccatcgccatggctgggatgccctcccctcacctgccctcggagcactcaagcgtgtctagcagcccagagcctgggatgcctgttatccagagcacttacggtgtgaaaggagaggagccacatatcaaagaagagatacaggccgaggacatcaatggagaaatttatgatgagtacgacgaggaagaggatgatccagatgtagattatgggagtgacagtgaaaaccatattgcaggacaagccaac

SEQ ID NO: 141 - protein, 804 aa, Homo sapiens
MGRMSSKQATSPFACAADGEDAMTQDLTSREKEEGSDQHVASHLPLHPIMHNKPHSEELPTLVSTIQQDADWDSVLSSQQRMESENNKLCSLYSFRNTSTSPHKPDEGSRDREIMTSVTFGTPERRKGSLADVVDTLKQKKLEEMTRTEQEDSSCMEKLLSKDWKEKMERLNTSELLGEIKGTPESLAEKERQLSTMITQLISLREQLLAAHDEQKKLAASQIEKQRQQMDLARQQQEQIARQQQQLLQQQHKINLLQQQIQVQGHMPPLMIPIFPHDQRTLAAAAAAQQGFLFPPGITYKPGDNYPVQFIPSTMAAAAASGLSPLQLQQLYAAQLASMQVSPGAKMPSTPQPPNTAGTVSPTGIKNEKRGTSPVTQVKDEAAAQPLNLSSRPKTAEPVKSPTSPTQNLFPASKTSPVNLPNKSSIPSPIGGSLGRGSSLGKWKSQHQEETYELDILSSLNSPALFGDQDTVMKAIQEARKMREQIQREQQQQQPHGVDGKLSSINNMGLNSCRNEKERTRFENLGPQLTGKSNEDGKLGPGVIDLTRPEDAEGSKAMNGSAAKLQQYYCWPTGGATVAEARVYRDARGRASSEPHIKRPMNAFMVWAKDERRKILQAFPDMHNSNISKILGSRWKSMSNQEKQPYYEEQARLSKIHLEKYPNYKYKPRPKRTCIVDGKKLRIGEYKQLMRSRRQEMRQFFTVGQQPQIPITTGTGVVYPGAITMATTTPSPQMTSDCSSTSASPEPSLPVIQSTYGMKTDGGSLAGNEMINGEDEMEMYDDYEDDPKSDYSSENEAPEAVSAN

SEQ ID NO: 142 - DNA, 2412 bp, Homo sapiens
  source 1..2412: mol_type = genomic DNA
atgggaagaatgtcttccaagcaagccacctctccatttgcctgtgcagctgatggagaggatgcaatgacccaggatttaacctcaagggaaaaggaagagggcagtgatcaacatgtggcctcccatctgcctctgcaccccataatgcacaacaaacctcactctgaggagctaccaacacttgtcagtaccattcaacaagatgctgactgggacagcgttctgtcatctcagcaaagaatggaatcagagaataataagttatgttccctatattccttccgaaatacctctacctcaccacataagcctgacgaagggagtcgggaccgtgagataatgaccagtgttacttttggaaccccagagcgccgcaaagggagtcttgccgatgtggtggacacactgaaacagaagaagcttgaggaaatgactcggactgaacaagaggattcctcctgcatggaaaaactactttcaaaagattggaaggaaaaaatggaaagactaaataccagtgaacttcttggagaaattaaaggtacacctgagagcctggcagaaaaagaacggcagctctccaccatgattacccagctgatcagtttacgggagcagctactggcagcgcatgatgaacagaaaaaactggcagcgtcacaaattgagaaacaacggcagcaaatggaccttgctcgccaacagcaagaacagattgcgagacaacagcagcaacttctgcaacagcagcacaaaattaatctcctgcagcaacagatccaggttcagggtcacatgcctccgctcatgatcccaatttttccacatgaccagcggactctggcagcagctgctgctgcccaacagggattcctcttcccccctggaataacatacaaaccaggtgataactaccccgtacagttcattccatcaacaatggcagctgctgctgcttctggactcagccctttacagctccagcagctctatgccgctcagctggccagcatgcaggtgtcacctggagcaaagatgccatcaactccacagccaccaaacacagcagggacggtctcacctactgggataaaaaatgaaaagagagggaccagccctgtaactcaagttaaggatgaagcagcagcacagcctctgaatctctcatcccgacccaagacagcagagcctgtaaagtccccaacgtctcccacccagaacctcttcccagccagcaaaaccagccctgtcaatctgccaaacaaaagcagcatccctagccccattggaggaagcctgggaagaggatcctctttaggtaaatggaaaagtcaacaccaggaagagacttacgaattagatatcctatctagtctcaactcccctgccctttttggggatcaggatacagtgatgaaagccattcaggaggcgcggaagatgcgagagcagatccagcgggagcaacagcagcaacagccacatggtgttgacgggaaactgtcctccataaataatatggggctgaacagctgcaggaatgaaaaggaaagaacgcgctttgagaatttggggccccagttaacgggaaagtcaaatgaagatggaaaactgggcccaggtgtcatcgaccttactcggccagaagatgcagagggaagtaaagcaatgaatggctctgcagctaaactacagcagtattattgttggccaacaggaggtgccactgtggctgaagcacgagtctacagggacgcccgcggccgtgccagcagcgagccacacattaagcgaccaatgaatgcattcatggtttgggcaaaggatgagaggagaaaaatccttcaggccttccccgacatgcataactccaacattagcaaaatcttaggatctcgctggaaatcaatgtccaaccaggagaagcaaccttattatgaagagcaggcccggctaagcaagatccacttagagaagtacccaaactataaatacaaaccccgaccgaaacgcacctgcattgttgatggcaaaaagcttcggattggggagtataagcaactgatgaggtctcggagacaggagatgaggcagttctttactgtggggcaacagcctcagattccaatcaccacaggaacaggtgttgtgtatcctggtgctatcactatggcaactaccacaccatcgcctcagatgacatctgactgctctagcacctcggccagcccggagcccagcctcccggtcatccagagcacttatggtatgaagacagatggcggaagcctagctggaaatgaaatgatcaatggagaggatgaaatggaaatgtatgatgactatgaagatgaccccaaatcagactatagcagtgaaaatgaagccccggaggctgtcagtgccaac

SEQ ID NO: 143 - protein, 622 aa, Homo sapiens
MSMRSPISAQLALDGVGTMVNCTIKSEEKKEPCHEAPQGSATAAEPQPGDPARASQDSADPQAPAQGNFRGSWDCSSPEGNGSPEPKRPGVSEAASGSQEKLDFNRNLKEVVPAIEKLLSSDWKERFLGRNSMEAKDVKGTQESLAEKELQLLVMIHQLSTLRDQLLTAHSEQKNMAAMLFEKQQQQMELARQQQEQIAKQQQQLIQQQHKINLLQQQIQQVNMPYVMIPAFPPSHQPLPVTPDSQLALPIQPIPCKPVEYPLQLLHSPPAPVVKRPGAMATHHPLQEPSQPLNLTAKPKAPELPNTSSSPSLKMSSCVPRPPSHGGPTRDLQSSPPSLPLGFLGEGDAVTKAIQDARQLLHSHSGALDGSPNTPFRKDLISLDSSPAKERLEDGCVHPLEEAMLSCDMDGSRHFPESRNSSHIKRPMNAFMVWAKDERRKILQAFPDMHNSSISKILGSRWKSMTNQEKQPYYEEQARLSRQHLEKYPDYKYKPRPKRTCIVEGKRLRVGEYKALMRTRRQDARQSYVIPPQAGQVQMSSSDVLYPRAAGMPLAQPLVEHYVPRSLDPNMPVIVNTCSLREEGEGTDDRHSVADGEMYRYSEDEDSEGEEKSDGELVVLTD

SEQ ID NO: 144 - DNA, 1866 bp, Homo sapiens
  source 1..1866: mol_type = genomic DNA
atgtccatgaggagccccatctctgcccagctggccctggatggcgttggcaccatggtgaactgcaccatcaagtcagaggagaagaaagagccttgccacgaggccccccagggctcagccactgccgctgaacctcagcctggagacccagcccgggcctcccaggatagtgctgacccccaagctccagcccaggggaatttcaggggctcctgggactgtagctctccagagggtaatgggtccccagaacccaagagaccaggagtgtcggaggctgcctctggaagccaggagaagctggacttcaaccgaaatttgaaagaagtggtgccagccatagagaagctgttgtccagtgactggaaggagaggtttctaggaaggaactctatggaagccaaagatgtcaaagggacccaagagagcctagcagagaaggagctccagcttctggtcatgattcaccagctgtccaccctgcgggaccagctcctgacagcccactcggagcagaagaacatggctgccatgctgtttgagaagcagcagcagcagatggagcttgcccggcagcagcaggagcagattgcaaagcagcagcagcagctgattcagcagcagcataagatcaacctccttcagcagcagatccagcaggttaacatgccttatgtcatgatcccagccttccccccaagccaccaacctctgcctgtcacccctgactcccagctggccttacccattcagcccattccctgcaaaccagtggagtatccgctgcagctgctgcacagcccccctgccccagtggtgaagaggcctggggccatggccacccaccaccccctgcaggagccctcccagcccctgaacctcacagccaagcccaaggcccccgagctgcccaacacctccagctccccaagcctgaagatgagcagctgtgtgccccgcccccccagccatggaggccccacgcgggacctgcagtccagccccccgagcctgcctctgggcttccttggtgaaggggacgctgtcaccaaagccatccaggatgctcggcagctgctgcacagccacagtggggccttggatggctcccccaacacccccttccgtaaggacctcatcagcctggactcatccccagccaaggagcggctggaggacggctgtgtgcacccactggaggaagccatgctgagctgcgacatggatggctcccgccacttccccgagtcccgaaacagcagccacatcaagaggcccatgaacgccttcatggtgtgggccaaggatgagcggaggaagatcctgcaagccttcccagacatgcacaactccagcatcagcaagatccttggatctcgctggaagtccatgaccaaccaggagaagcagccctactatgaggaacaggcgcggctgagccggcagcacctggagaagtatcctgactacaagtacaagccgcggcccaagcgcacctgcatcgtggagggcaagcggctgcgcgtgggagagtacaaggccctgatgaggacccggcgtcaggatgcccgccagagctacgtgatccccccgcaggctggccaggtgcagatgagctcctcagatgtcctgtaccctcgggcagcaggcatgccgctggcacagccactggtggagcactatgtccctcgtagcctggaccccaacatgcctgtgatcgtcaacacctgcagcctcagagaggagggtgagggcacagatgacaggcactcggtggctgatggcgagatgtaccggtacagcgaggacgaggactcggagggcgaagagaagagcgatggggagttggtggtgctcacagac

SEQ ID NO: 145 - protein, 446 aa, Homo sapiens [sequence omitted]

SEQ ID NO: 146 - DNA, 1338 bp, Homo sapiens
  source 1..1338: mol_type = genomic DNA
atgctggacatgagcgaggcccgctcccagccgccctgcagcccgtccggcaccgccagctccatgtcgcacgtggaggactcggactcggacgcgccgccgtctcccgccggctccgagggcctgggccgcgcgggggtcgcggtggggggcgcccggggcgacccggcggaggcggcggacgagcgcttcccggcctgcatccgcgacgccgtgtcgcaggtgctcaagggctacgactggagtctggtgcccatgccggtgcgcggcggcggcggcggcgcgctcaaagccaagccgcatgtgaagcggcccatgaacgcattcatggtgtgggcgcaggcggcgcgccgcaagctggccgaccagtacccgcacctgcacaacgccgagctcagcaagacgctgggcaagctgtggcgcttgctgagcgagagcgagaagcggcccttcgtggaggaggcagagcgccttcgcgtgcagcacaagaaggaccaccccgactacaagtaccagccacggcgcaggaagagcgccaaagccggccacagcgactccgactcgggcgcggagctgggaccccaccctggcggcggtgccgtgtacaaggctgaagcagggcttggagatgggcaccaccatggcgaccacacagggcagacccacgggccgcccaccccgcccaccacccccaagacggagctgcagcaggcgggcgccaagccggagctgaagctggagggacgccggccggtggacagcgggcgccagaacatcgacttcagcaatgtggacatctcggagctcagcagcgaggtcatgggcaccatggacgccttcgacgtccacgagttcgaccagtacctgcccctgggcggccccgccccacccgagccgggccaggcctatgggggcgcctacttccacgccggggcgtcccccgtgtgggcccacaagagtgccccgtcggcctccgcgtcgcccaccgagacgggtcccccacggccgcacatcaagacggagcagccgagccccggccactacggcgaccagccccgaggctcgcccgactacggttcctgcagcggccagtccagcgccaccccggccgcccccgccggccccttcgccggctcacagggcgactatggcgacctgcaggcctccagctactatggtgcctaccctggctacgcacccggcctctaccagtacccctgcttccactcgccgcgccggccctacgcctcacccctgctcaacggcctggccctgccgcccgcccacagccccaccagtcactgggaccagccggtgtacaccaccctgaccaggccc

SEQ ID NO: 147 - protein, 509 aa, Homo sapiens
MNLLDPFMKMTDEQEKGLSGAPSPTMSEDSAGSPCPSGSGSDTENTRPQENTFPKGEPDLKKESEEDKFPVCIREAVSQVLKGYDWTLVPMPVRVNGSSKNKPHVKRPMNAFMVWAQAARRKLADQYPHLHNAELSKTLGKLWRLLNESEKRPFVEEAERLRVQHKKDHPDYKYQPRRRKSVKNGQAEAEEATEQTHISPNAIFKALQADSPHSSSGMSEVHSPGEHSGQSQGPPTPPTTPKTDVQPGKADLKREGRPLPEGGRQPPIDFRDVDIGELSSDVISNIETFDVNEFDQYLPPNGHPGVPATHGQVTYTGSYGISSTAATPASAGHVWMSKQQAPPPPPQQPPQAPPAPQAPPQPQAAPPQQPAAPPQQPQAHTLTTLSSEPGQSQRTHIKTEQLSPSHYSEQQQHSPQQIAYSPFNLPHYSPSYPPITRSQYDYTDHQNSSSYYSHAAGQGTGLYSTFTYMNPAQRPMYTPIADTSGVPSIPQTHSPQHWEQPVYTQLTRP

SEQ ID NO: 148 - DNA, 1527 bp, Homo sapiens
  source 1..1527: mol_type = genomic DNA
atgaatctcctggaccccttcatgaagatgaccgacgagcaggagaagggcctgtccggcgcccccagccccaccatgtccgaggactccgcgggctcgccctgcccgtcgggctccggctcggacaccgagaacacgcggccccaggagaacacgttccccaagggcgagcccgatctgaagaaggagagcgaggaggacaagttccccgtgtgcatccgcgaggcggtcagccaggtgctcaaaggctacgactggacgctggtgcccatgccggtgcgcgtcaacggctccagcaagaacaagccgcacgtcaagcggcccatgaacgccttcatggtgtgggcgcaggcggcgcgcaggaagctcgcggaccagtacccgcacttgcacaacgccgagctcagcaagacgctgggcaagctctggagacttctgaacgagagcgagaagcggcccttcgtggaggaggcggagcggctgcgcgtgcagcacaagaaggaccacccggattacaagtaccagccgcggcggaggaagtcggtgaagaacgggcaggcggaggcagaggaggccacggagcagacgcacatctcccccaacgccatcttcaaggcgctgcaggccgactcgccacactcctcctccggcatgagcgaggtgcactcccccggcgagcactcggggcaatcccagggcccaccgaccccacccaccacccccaaaaccgacgtgcagccgggcaaggctgacctgaagcgagaggggcgccccttgccagaggggggcagacagccccctatcgacttccgcgacgtggacatcggcgagctgagcagcgacgtcatctccaacatcgagaccttcgatgtcaacgagtttgaccagtacctgccgcccaacggccacccgggggtgccggccacgcacggccaggtcacctacacgggcagctacggcatcagcagcaccgcggccaccccggcgagcgcgggccacgtgtggatgtccaagcagcaggcgccgccgccacccccgcagcagcccccacaggccccgccggccccgcaggcgcccccgcagccgcaggcggcgcccccacagcagccggcggcacccccgcagcagccacaggcgcacacgctgaccacgctgagcagcgagccgggccagtcccagcgaacgcacatcaagacggagcagctgagccccagccactacagcgagcagcagcagcactcgccccaacagatcgcctacagccccttcaacctcccacactacagcccctcctacccgcccatcacccgctcacagtacgactacaccgaccaccagaactccagctcctactacagccacgcggcaggccagggcaccggcctctactccaccttcacctacatgaaccccgctcagcgccccatgtacacccccatcgccgacacctctggggtcccttccatcccgcagacccacagcccccagcactgggaacaacccgtctacacacagctcactcgacct

SEQ ID NO: 149 - protein, 466 aa, Homo sapiens
MAEEQDLSEVELSPVGSEEPRCLSPGSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGEAEQGGTAAIQAHYKSAHLDHRHPGEGSPMSDGNPEHPSGQSHGPPTPPTTPKTELQSGKADPKRDGRSMGEGGKPHIDFGNVDIGEISHEVMSNMETFDVAELDQYLPPNGHPGHVSSYSAAGYGLGSALAVASGHSAWISKPPGVALPTVSPPGVDAKAQVKTETAGPQGPPHYTDQPSTSQIAYTSLSLPHYGSAFPSISRPQFDYSDHQPSGPYYGHSGQASGLYSAFSYMGPSQRPLYTAISDPSPSGPQSHSPTHWEQPVYTTLSRP

SEQ ID NO: 150 - DNA, 1398 bp, Homo sapiens
  source 1..1398: mol_type = genomic DNA
atggcggaggagcaggacctatcggaggtggagctgagccccgtgggctcggaggagccccgctgcctgtccccggggagcgcgccctcgctagggcccgacggcggcggcggcggatcgggcctgcgagccagcccggggccaggcgagctgggcaaggtcaagaaggagcagcaggacggcgaggcggacgatgacaagttccccgtgtgcatccgcgaggccgtcagccaggtgctcagcggctacgactggacgctggtgcccatgcccgtgcgcgtcaacggcgccagcaaaagcaagccgcacgtcaagcggcccatgaacgccttcatggtgtgggctcaggcagcgcgcaggaagctcgcggaccagtacccgcacctgcacaacgctgagctcagcaagacgctgggcaagctctggaggctgctgaacgaaagtgacaagcgccccttcatcgaggaggctgagcggctccgtatgcagcacaagaaagaccacccggactacaagtaccagcccaggcggcggaagaacgggaaggccgcccagggcgaggcggagtgccccggtggggaggccgagcaaggtgggaccgccgccatccaggcccactacaagagcgcccacttggaccaccggcacccaggagagggctcccccatgtcagatggtaaccccgagcacccctcaggccagagccatggcccacccacccctccaaccaccccgaagacagagctgcagtcgggcaaggcagacccgaagcgggacgggcgctccatgggggagggcgggaagcctcacatcgacttcggcaacgtggacattggtgagatcagccacgaggtaatgtccaacatggagacctttgatgtggctgagttggaccagtacctgccgcccaatgggcacccaggccatgtgagcagctactcagcagccggctatgggctgggcagtgccctggccgtggccagtggacactccgcctggatctccaagccaccaggcgtggctctgcccacggtctcaccacctggtgtggatgccaaagcccaggtgaagacagagaccgcggggccccaggggcccccacactacaccgaccagccatccacctcacagatcgcctacacctccctcagcctgccccactatggctcagccttcccctccatctcccgcccccagtttgactactctgaccatcagccctcaggaccctattatggccactcgggccaggcctctggcctctactcggccttctcctatatggggccctcgcagcggcccctctacacggccatctctgaccccagcccctcagggccccagtcccacagccccacacactgggagcagccagtatatacgacactgtcccggccc

SEQ ID NO: 151 - protein, 388 aa, Homo sapiens [sequence omitted]

SEQ ID NO: 152 - DNA, 1164 bp, Homo sapiens
  source 1..1164: mol_type = genomic DNA
atggcttcgctgctgggagcctacccttggcccgagggtctcgagtgcccggccctggacgccgagctgtcggatggacaatcgccgccggccgtcccccggcccccgggggacaagggctccgagagccgtatccggcggcccatgaacgccttcatggtttgggccaaggacgagaggaaacggctggcagtgcagaacccggacctgcacaacgccgagctcagcaagatgctgggaaagtcgtggaaggcgctgacgctgtcccagaagaggccgtacgtggacgaggcggagcggctgcgcctgcagcacatgcaggactaccccaactacaagtaccggccgcgcaggaagaagcaggccaagcggctgtgcaagcgcgtggacccgggcttccttctgagctccctctcccgggaccagaacgccctgccggagaagagaagcggcagccggggggcgctgggggagaaggaggacaggggtgagtactcccccggcactgccctgcccagcctccggggctgctaccacgaggggccggctggtggtggcggcggcggcaccccgagcagtgtggacacgtacccgtacgggctgcccacacctcctgaaatgtctcccctggacgtgctggagccggagcagaccttcttctcctccccctgccaggaggagcatggccatccccgccgcatcccccacctgccagggcacccgtactcaccggagtacgccccaagccctctccactgtagccaccccctgggctccctggcccttggccagtcccccggcgtctccatgatgtcccctgtacccggctgtcccccatctcctgcctattactccccggccacctaccacccactccactccaacctccaagcccacctgggccagctttccccgcctcctgagcaccctggcttcgacgccctggatcaactgagccaggtggaactcctgggggacatggatcgcaatgaattcgaccagtatttgaacactcctggccacccagactccgccacaggggccatggccctcagtgggcatgttccggtctcccaggtgacaccaacgggtcccacagagaccagcctcatctccgtcctggctgatgccacggccacgtactacaacagctacagtgtgtca

SEQ ID NO: 153 - protein, 414 aa, Homo sapiens [sequence omitted]

SEQ ID NO: 154 - DNA, 1242 bp, Homo sapiens
  source 1..1242: mol_type = genomic DNA
atgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgcaggcggccgcgctgggccccgagggcggccgcgtggccatggacggcctgggcctccagttccccgagcagggcttccccgccggcccgccgctgctgcctccgcacatgggcggccactaccgcgactgccagagtctgggcgcgcctccgctcgacggctacccgttgcccacgcccgacacgtccccgctggacggcgtggaccccgacccggctttcttcgccgccccgatgcccggggactgcccggcggccggcacctacagctacgcgcaggtctcggactacgctggccccccggagcctcccgccggtcccatgcacccccgactcggcccagagcccgcgggtccctcgattccgggcctcctggcgccacccagcgcccttcacgtgtactacggcgcgatgggctcgcccggggcgggcggcgggcgcggcttccagatgcagccgcaacaccagcaccagcaccagcaccagcaccaccccccgggccccggacagccgtcgccccctccggaggcactgccctgccgggacggcacggaccccagtcagcccgccgagctcctcggggaggtggaccgcacggaatttgaacagtatctgcacttcgtgtgcaagcctgagatgggcctcccctaccaggggcatgactccggtgtgaatctccccgacagccacggggccatttcctcggtggtgtccgacgccagctccgcggtatattactgcaactatcctgacgtg

SEQ ID NO: 155 - protein, 387 aa, Homo sapiens
MQRSPPGYGAQDDPPARRDCAWAPGHGAAADTRGLAAGPAALAAPAAPASPPSPQRSPPRSPEPGRYGLSPAGRGERQAADESRIRRPMNAFMVWAKDERKRLAQQNPDLHNAVLSKMLGKAWKELNAAEKRPFVEEAERLRVQHLRDHPNYKYRPRRKKQARKARRLEPGLLLPGLAPPQPPPEPFPAASGSARAFRELPPLGAEFDGLGLPTPERSPLDGLEPGEAAFFPPPAAPEDCALRPFRAPYAPTELSRDPGGCYGAPLAEALRTAPPAAPLAGLYYGTLGTPGPYPGPLSPPPEAPPLESAEPLGPAADLWADVDLTEFDQYLNCSRTRPDAPGLPYHVALAKLGPRAMSCPEESSLISALSDASSAVYYSACISGSGP

SEQ ID NO: 156 - DNA, 1161 bp, Homo sapiens
  source 1..1161: mol_type = genomic DNA
atgcagagatcgccgcccggctacggcgcacaggacgacccgcccgcccgccgcgactgtgcatgggccccgggacacggggccgccgctgacacgcgcggcctcgccgccggccccgccgccctcgccgcgcccgccgcgcccgcctcgccgcccagcccgcagcgcagtcccccgcgcagccccgagccggggcgctatggcctcagcccggccggccgcggggaacgccaggcggcagacgagtcgcgcatccggcggcccatgaacgccttcatggtgtgggcaaaggacgagcgcaagcggctggctcagcagaacccggacctgcacaacgcggtgctcagcaagatgctgggcaaagcgtggaaggagctgaacgcggcggagaagcggcccttcgtggaggaagccgaacggctgcgcgtgcagcacttgcgcgaccaccccaactacaagtaccggccgcgccgcaagaagcaggcgcgcaaggcccggcggctggagcccggcctcctgctcccgggattagcgcccccgcagccaccgcccgagcctttccccgcggcgtctggctcggctcgcgccttccgcgagctgcccccgctgggcgccgagttcgacggcctggggctgcccacgcccgagcgctcgcctctggacggcctggagcccggcgaggctgccttcttcccaccgcccgcggcgcccgaggactgcgcgctgcggcccttccgcgcgccctacgcgcccaccgagttgtcgcgggaccccggcggttgctacggggctcccctggcggaggcgctcaggaccgcgccccccgcggcgccgctcgctggcctgtactacggcaccctgggcacgcccggcccgtaccccggcccgctgtcgccgccgcccgaggccccgccgctggagagcgccgagccgctggggcccgccgccgatctgtgggccgacgtggacctcaccgagttcgaccagtacctcaactgcagccggactcggcccgacgcccccgggctcccgtaccacgtggcactggccaaactgggcccgcgcgccatgtcctgcccagaggagagcagcctgatctccgcgctgtcggacgccagcagcgcggtctattacagcgcgtgcatctccggcagcggaccg

SEQ ID NO: 157 - protein, 233 aa, Homo sapiens [sequence omitted]

SEQ ID NO: 158 - DNA, 699 bp, Homo sapiens
  source 1..699: mol_type = genomic DNA
atggcgctaccaggctcctcacaggaccaggcctggagcctggagcctccggctgccacggctgctgcctcctcatcttcgggaccccaggagcgggagggcgctgggagccccgcggcccccgggacgctgcccctggagaaggtgaagcggccgatgaacgcgttcatggtgtggagctccgctcagcgccgccagatggcgcagcagaaccccaagatgcacaactccgagatctccaagcgcctgggcgcgcagtggaagctgctggacgaggacgagaagcggcccttcgtggaggaggccaagcggctccgcgcccgacacctgcgcgactaccccgactacaagtaccggcctcggcgcaaggccaagagctcgggcgccggaccttcccgctgcggacagggaagaggcaacctggccagcggcggcccgctctgggggccggggtacgcgaccacccaaccgagcagaggctttgggtacagaccccccagctactcgacagcctacctgcctggcagctatggctcttcccactgcaaactggaagccccctcaccgtgctccctccctcagagtgaccctaggctccagggggaactgctgcccacctatacccactacctgccccctggctctcccactccatacaaccctccccttgctggtgcccccatgcccctaacccacctc

SEQ ID NO: 159 - protein, 753 aa, Homo sapiens [sequence omitted]

SEQ ID NO: 160 - DNA, 2259 bp, Homo sapiens
  source 1..2259: mol_type = genomic DNA
atggagagagccagacccgagccgccgcctcagccgcgcccgttgcgtcccgctccgcccccgctgccggtcgagggcacctccttttgggcagcagccatggagccccctccgtcgtctcccacactgagcgcggcagccagtgcgaccttggcctcgtcgtgcggggaggcagtggcgtccggcttacagcccgcggtgcggcggctgctgcaggtgaagccagagcaggtgttgctgctaccacagcctcaggcccagaacgaggaagccgctgcctcgtccgcgcaggcgcggctgttgcagttcaggcccgacctgcggctcctgcagccgccgacagcgtcagacggcgccacctccaggcccgagttgcacccggtgcagcccctggcgctgcatgtcaaggccaagaagcagaagctggggcccagcctggatcagtcagtggggcctcgaggggccgtcgaaaccggtcctagagcctccagggtggtcaagttggaaggccccgggccggccctcggctacttccgaggggacgagaagggcaagctggaggcggaggaggtcatgagagactcgatgcaaggcggggcaggcaaaagcccggcagccatccgagaaggtgtgatcaaaacggaggaacccgagagactcctcgaggactgcaggctcggcgcggagcccgcgtccaatggcctggttcatggcagcgcggaggtcatcttggccccaacgtccggtgcctttgggccgcaccagcaagaccttaggatccctttgacgctccacacggtcccccctggggcccggatccagtttcagggagctccgccttcagagctgataagattgaccaaggtccccctgacaccagtgcctactaaaatgcagtccctactggagccttctgtaaaaattgaaaccaaagatgtcccgctcaccgtgttgccctcagatgcaggcataccagatactcccttcagtaaggacagaaatggtcatgtgaagcgacccatgaacgcatttatggtttgggcaaggatccaccgaccagcactagccaaagctaacccagcagccaacaatgcagaaatcagtgtccagcttgggttagagtggaacaaacttagtgaagaacaaaagaaaccctattacgatgaagcacaaaagattaaggaaaagcacagagaggaatttcctggttgggtttatcagcctcgtccagggaagcgaaaacgattccctctaagtgtttccaatgtattttctggtaccacaaagaatatcatctctacaaatcctacaacagtttatccttaccgctcacctacgtactctgtggtaattcccagcctacagaatcccatcactcatccagttggtgaaacctcacctgctatccagctgcccacacctgcagtccagagcccaagccctgtcacacttttccagcccagcgtctccagtgctgctcaggtggctgtccaggatccaagtctacctgtctatccagcactcccaccccaacgctttactgggccttcccaaacagacactcatcagctgcattctgaagccactcacactgtgaagcaacccactcctgtctctctagagagcgccaacaggatttcaagtagtgcaagtactgcccatgccagatttgcaacttcgaccatccaacctcctagggagtattccagcgtttccccttgtcccagaagtgctccaatcccccaggcttctcccattccacacccacatgtctaccagccccctccccttggccatccagccacactgttcgggacaccaccaagattctcttttcatcacccttacttcctacccggacctcactacttcccatcaagtacatgcccttacagtcggcctccctttggctatggaaattttccgagttcaatgccagaatgccttagttattatgaagacaggtacccaaaacatgagggtatcttttcaactttaaatagagactattcttttagagactactcaagtgaatgcacacacagtgaaaattctcggagttgtgagaacatgaatggaacttcttactataacagtcatagccacagtggggaagaaaacttaaaccctgtgcctcagctggacattggaaccttggagaatgtcttcacagccccgacatcaactccttctagcatccagcaagtcaatgtcaccgacagtgatgaggaggaagaagaaaaagtgctcagggattta

SEQ ID NO: 161 - protein, 251 aa, Homo sapiens
MPSPRPRGSPPPAPSGSRVRPPRSGRSPAPRSPTGPNTPRAPGRFESPFSVEAILARPDPCAPAASQPSGSACVHPAFWTAASLCATGGLPWACPTSWLPAYLSVGFYPVPGPRVAPVCGLLGFGVTGLELAHCSGLWAFPDWAPTEDLQDTERQQKRVRTMFNLEQLEELEKVFAKQHNLVGKKRAQLAARLKLTENQVRVWFQNRRVKYQKQQKLRAAVTSAEAASLDEPSSSSIASIQSDDAESGVDG

SEQ ID NO: 162 - DNA, 753 bp, Homo sapiens
  source 1..753: mol_type = genomic DNA
atgcctagccccaggccgcgaggcagcccgccacccgctccctcgggctctcgggtccgacctccgcgctctggccgctctccggcgcccaggtcccctactggcccgaacacgccccgcgctcccggacgcttcgagtcccctttctcggtcgaggccatcctggcgaggcccgacccctgcgcgccggcggcctcccagccgtcgggctccgcctgcgtccacccggccttctggaccgctgcttccctgtgcgccaccgggggtctgccctgggcttgcccgacatcgtggctgcccgcctacctgagcgtaggtttttaccctgtgccagggccgcgcgtggctcccgtctgcggcctgctgggcttcggcgtcacagggttggagctggctcactgctcaggactctgggccttcccagactgggccccaacggaggacctacaggacactgagagacagcaaaagagagtccgaactatgtttaacttggagcagctggaagagttggagaaagtgtttgcaaaacagcacaatctggtggggaagaagagagcccagctggcagctcggctcaaacttacagagaaccaggtgagagtctggttccagaaccgcagggtcaagtatcagaagcagcaaaagctgagggcagcagttacatctgccgaggctgcctccctggatgagccttccagcagctccatcgccagtatccagagtgatgatgccgagtcaggagtggacggc

SEQ ID NO: 163 - protein, 317 aa, Homo sapiens
MAKNPPENCEDCHILNAEAFKSKKICKSLKICGLVFGILTLTLIVLFWGSKHFWPEVPKKAYDMEHTFYSSGEKKKIYMEIDPVTRTEIFRSGNGTDETLEVHDFKNGYTGIYFVGLQKCFIKTQIKVIPEFSEPEEEIDENEEITTTFFEQSVIWVPAEKPIENRDFLKNSKILEICDNVTMYWINPTLISVSELQDFEEEGEDLHFPANEKKGIEQNEQWVVPQVKVEKTRHARQASEEELPINDYTENGIEFDPMLDERGYCCIYCRRGNRYCRRVCEPLLGYYPYPYCYQGGRVICRVIMPCNWWVARMLGRV

SEQ ID NO: 164 - DNA, 951 bp, Homo sapiens
  source 1..951: mol_type = genomic DNA
atggcaaagaatcctccagagaattgtgaagactgtcacattctaaatgcagaagcttttaaatccaagaaaatatgtaaatcacttaagatttgtggactggtgtttggtatcctgaccctaactctaattgtcctgttttgggggagcaagcacttctggccggaggtacccaaaaaagcctatgacatggagcacactttctacagcagtggagagaagaagaagatttacatggaaattgatcctgtgaccagaactgaaatattcagaagcggaaatggcactgatgaaacattggaagtacacgactttaaaaacggatacactggcatctacttcgtgggtcttcaaaaatgttttatcaaaactcagattaaagtgattcctgaattttctgaaccagaagaggaaatagatgagaatgaagaaattaccacaactttctttgaacagtcagtgatttgggtcccagcagaaaagcctattgaaaaccgagattttcttaaaaattccaaaattctggagatttgtgataacgtgaccatgtattggatcaatcccactctaatatcagtttctgagttacaagactttgaggaggagggagaagatcttcactttcctgccaacgaaaaaaaagggattgaacaaaatgaacagtgggtggtccctcaagtgaaagtagagaagacccgtcacgccagacaagcaagtgaggaagaacttccaataaatgactatactgaaaatggaatagaatttgatcccatgctggatgagagaggttattgttgtatttactgccgtcgaggcaaccgctattgccgccgcgtctgtgaacctttactaggctactacccatatccatactgctaccaaggaggacgagtcatctgtcgtgtcatcatgccttgtaactggtgggtggcccgcatgctggggagggtc

SEQ ID NO: 165 - protein, 716 aa, Homo sapiens
MPSTSFPVPSKFPLGPAAAVFGRGETLGPAPRAGGTMKSAEEEHYGYASSNVSPALPLPTAHSTLPAPCHNLQTSTPGIIPPADHPSGYGAALDGGPAGYFLSSGHTRPDGAPALESPRIEITSCLGLYHNNNQFFHDVEVEDVLPSSKRSPSTATLSLPSLEAYRDPSCLSPASSLSSRSCNSEASSYESNYSYPYASPQTSPWQSPCVSPKTTDPEEGFPRGLGACTLLGSPRHSPSTSPRASVTEESWLGARSSRPASPCNKRKYSLNGRQPPYSPHHSPTPSPHGSPRVSVTDDSWLGNTTQYTSSAIVAAINALTTDSSLDLGDGVPVKSRKTTLEQPPSVALKVEPVGEDLGSPPPPADFAPEDYSSFQHIRKGGFCDQYLAVPQHPYQWAKPKPLSPTSYMSPTLPALDWQLPSHSGPYELRIEVQPKSHHRAHYETEGSRGAVKASAGGHPIVQLHGYLENEPLMLQLFIGTADDRLLRPHAFYQVHRITGKTVSTTSHEAILSNTKVLEIPLLPENSMRAVIDCAGILKLRNSDIELRKGETDIGRKNTRVRLVFRVHVPQPSGRTLSLQVASNPIECSQRSAQELPLVEKQSTDSYPVVGGKKMVLSGHNFLQDSKVIFVEKAPDGHHVWEMEAKTDRDLCKPNSLVVEIPPFRNQRITSPVHVSFYVCNGKRKRSQYQRFTYLPANGNAIFLTVSREHERVGCFF

SEQ ID NO: 166 - DNA, 2148 bp, Homo sapiens
  source 1..2148: mol_type = genomic DNA
atgccaagcaccagctttccagtcccttccaagtttccacttggccctgcggctgcggtcttcgggagaggagaaactttggggcccgcgccgcgcgccggcggcaccatgaagtcagcggaggaagaacactatggctatgcatcctccaacgtcagccccgccctgccgctccccacggcgcactccaccctgccggccccgtgccacaaccttcagacctccacaccgggcatcatcccgccggcggatcacccctcggggtacggggcagctttggacggtgggcccgcgggctacttcctctcctccggccacaccaggcctgatggggcccctgccctggagagtcctcgcatcgagataacctcgtgcttgggcctgtaccacaacaataaccagtttttccacgatgtggaggtggaagacgtcctccctagctccaaacggtccccctccacggccacgctgagtctgcccagcctggaggcctacagagacccctcgtgcctgagcccggccagcagcctgtcctcccggagctgcaactcagaggcctcctcctacgagtccaactactcgtacccgtacgcgtccccccagacgtcgccatggcagtctccctgcgtgtctcccaagaccacggaccccgaggagggctttccccgcgggctgggggcctgcacactgctgggttccccgcggcactccccctccacctcgccccgcgccagcgtcactgaggagagctggctgggtgcccgctcctccagacccgcgtccccgtgcaacaagaggaagtacagcctcaacggccggcagccgccctactcaccccaccactcgcccacgccgtccccgcacggctccccgcgggtcagcgtgaccgacgactcgtggttgggcaacaccacccagtacaccagctcggccatcgtggccgccatcaacgcgctgaccaccgacagcagcctggacctgggagatggcgtccctgtcaagtcccgcaagaccaccctggagcagccgccctcagtggcgctcaaggtggagcccgtcggggaggacctgggcagccccccgcccccggccgacttcgcgcccgaagactactcctctttccagcacatcaggaagggcggcttctgcgaccagtacctggcggtgccgcagcacccctaccagtgggcgaagcccaagcccctgtcccctacgtcctacatgagcccgaccctgcccgccctggactggcagctgccgtcccactcaggcccgtatgagcttcggattgaggtgcagcccaagtcccaccaccgagcccactacgagacggagggcagccggggggccgtgaaggcgtcggccggaggacaccccatcgtgcagctgcatggctacttggagaatgagccgctgatgctgcagcttttcattgggacggcggacgaccgcctgctgcgcccgcacgccttctaccaggtgcaccgcatcacagggaagaccgtgtccaccaccagccacgaggccatcctctccaacaccaaagtcctggagatcccactcctgccggagaacagcatgcgagccgtcattgactgtgccggaatcctgaaactcagaaactccgacattgaacttcggaaaggagagacggacatcgggaggaagaacacacgggtacggctggtgttccgcgttcacgtcccgcaacccagcggccgcacgctgtccctgcaggtggcctccaaccccatcgaatgctcccagcgctcagctcaggagctgcctctggtggagaagcagagcacggacagctatccggtcgtgggcgggaagaagatggtcctgtctggccacaacttcctgcaggactccaaggtcattttcgtggagaaagccccagatggccaccatgtctgggagatggaagcgaaaactgaccgggacctgtgcaagccgaattctctggtggttgagatcccgccatttcggaatcagaggataaccagccccgttcacgtcagtttctacgtctgcaacgggaagagaaagcgaagccagtaccagcgtttcacctaccttcccgccaacggtaacgccatctttctaaccgtaagccgtgaacatgagcgcgtggggtgctttttc

SEQ ID NO: 167 - protein, 921 aa, Homo sapiens
MNAPERQPQPDGGDAPGHEPGGSPQDELDFSILFDYEYLNPNEEEPNAHKVASPPSGPAYPDDVLDYGLKPYSPLASLSGEPPGRFGEPDRVGPQKFLSAAKPAGASGLSPRIEITPSHELIQAVGPLRMRDAGLLVEQPPLAGVAASPRFTLPVPGFEGYREPLCLSPASSGSSASFISDTFSPYTSPCVSPNNGGPDDLCPQFQNIPAHYSPRTSPIMSPRTSLAEDSCLGRHSPVPRPASRSSSPGAKRRHSCAEALVALPPGASPQRSRSPSPQPSSHVAPQDHGSPAGYPPVAGSAVIMDALNSLATDSPCGIPPKMWKTSPDPSPVSAAPSKAGLPRHIYPAVEFLGPCEQGERRNSAPESILLVPPTWPKPLVPAIPICSIPVTASLPPLEWPLSSQSGSYELRIEVQPKPHHRAHYETEGSRGAVKAPTGGHPVVQLHGYMENKPLGLQIFIGTADERILKPHAFYQVHRITGKTVTTTSYEKIVGNTKVLEIPLEPKNNMRATIDCAGILKLRNADIELRKGETDIGRKNTRVRLVFRVHIPESSGRIVSLQTASNPIECSQRSAHELPMVERQDTDSCLVYGGQQMILTGQNFTSESKVVFTEKTTDGQQIWEMEATVDKDKSQPNMLFVEIPEYRNKHIRTPVKVNFYVINGKRKRSQPQHFTYHPVPAIKTEPTDEYDPTLICSPTHGGLGSQPYYPQHPMVAESPSCLVATMAPCQQFRTGLSSPDARYQQQNPAAVLYQRSKSLSPSLLGYQQPALMAAPLSLADAHRSVLVHAGSQGQSSALLHPSPTNQQASPVIHYSPTNQQLRCGSHQEFQHIMYCENFAPGTTRPGPPPVSQGQRLSPGSYPTVIQQQNATSQRAAKNGPPVSDQKEVLPAGVTIKQEQNLDQTYLDDELIDTHLSWIQNIL

SEQ ID NO: 168 - DNA, 2763 bp, Homo sapiens
  source 1..2763: mol_type = genomic DNA
atgaacgcccccgagcggcagccccaacccgacggcggggacgccccaggccacgagcctgggggcagcccccaagacgagcttgacttctccatcctcttcgactatgagtatttgaatccgaacgaagaagagccgaatgcacataaggtcgccagcccaccctccggacccgcataccccgatgatgtcctggactatggcctcaagccatacagcccccttgctagtctctctggcgagccccccggccgattcggagagccggatagggtagggccgcagaagtttctgagcgcggccaagccagcaggggcctcgggcctgagccctcggatcgagatcactccgtcccacgaactgatccaggcagtggggcccctccgcatgagagacgcgggcctcctggtggagcagccgcccctggccggggtggccgccagcccgaggttcaccctgcccgtgcccggcttcgagggctaccgcgagccgctttgcttgagccccgctagcagcggctcctctgccagcttcatttctgacaccttctccccctacacctcgccctgcgtctcgcccaataacggcgggcccgacgacctgtgtccgcagtttcaaaacatccctgctcattattcccccagaacctcgccaataatgtcacctcgaaccagcctcgccgaggacagctgcctgggccgccactcgcccgtgccccgtccggcctcccgctcctcatcgcctggtgccaagcggaggcattcgtgcgccgaggccttggttgccctgccgcccggagcctcaccccagcgctcccggagcccctcgccgcagccctcatctcacgtggcaccccaggaccacggctccccggctgggtacccccctgtggctggctctgccgtgatcatggatgccctgaacagcctcgccacggactcgccttgtgggatcccccccaagatgtggaagaccagccctgacccctcgccggtgtctgccgccccatccaaggccggcctgcctcgccacatctacccggccgtggagttcctggggccctgcgagcagggcgagaggagaaactcggctccagaatccatcctgctggttccgcccacttggcccaagccgctggtgcctgccattcccatctgcagcatcccagtgactgcatccctccctccacttgagtggccgctgtccagtcagtcaggctcttacgagctgcggatcgaggtgcagcccaagccacatcaccgggcccactatgagacagaaggcagccgaggggctgtcaaagctccaactggaggccaccctgtggttcagctccatggctacatggaaaacaagcctctgggacttcagatcttcattgggacagctgatgagcggatccttaagccgcacgccttctaccaggtgcaccgaatcacggggaaaactgtcaccaccaccagctatgagaagatagtgggcaacaccaaagtcctggagatacccttggagcccaaaaacaacatgagggcaaccatcgactgtgcggggatcttgaagcttagaaacgccgacattgagctgcggaaaggcgagacggacattggaagaaagaacacgcgggtgagactggttttccgagttcacatcccagagtccagtggcagaatcgtctctttacagactgcatctaaccccatcgagtgctcccagcgatctgctcacgagctgcccatggttgaaagacaagacacagacagctgcctggtctatggcggccagcaaatgatcctcacggggcagaactttacatccgagtccaaagttgtgtttactgagaagaccacagatggacagcaaatttgggagatggaagccacggtggataaggacaagagccagcccaacatgctttttgttgagatccctgaatatcggaacaagcatatccgcacacctgtaaaagtgaacttctacgtcatcaatgggaagagaaaacgaagtcagcctcagcactttacctaccacccagtcccagccatcaagacggagcccacggatgaatatgaccccactctgatctgcagccccacccatggaggcctggggagccagccttactacccccagcacccgatggtggccgagtccccctcctgcctcgtggccaccatggctccctgccagcagttccgcacggggctctcatcccctgacgcccgctaccagcaacagaacccagcggccgtactctaccagcggagcaagagcctgagccccagcctgctgggctatcagcagccggccctcatggccgccccgctgtcccttgcggacgctcaccgctctgtgctggtgcacgccggctcccagggccagagctcagccctgctccacccctctccgaccaaccagcaggcctcgcctgtgatccactactcacccaccaaccagcagctgcgctgcggaagccaccaggagttccagcacatcatgtactgcgagaatttcgcaccaggcaccaccagacctggcccgcccccggtcagtcaaggtcagaggctgagcccgggttcctaccccacagtcattcagcagcagaatgccacgagccaaagagccgccaaaaacggacccccggtcagtgaccaaaaggaagtattacctgcgggggtgaccattaaacaggagcagaacttggaccagacctacttggatgatgagctgatagacacacaccttagctggatacaaaacatatta

SEQ ID NO: 169 - protein, 1068 aa, Homo sapiens
MTTANCGAHDELDFKLVFGEDGAPAPPPPGSRPADLEPDDCASIYIFNVDPPPSTLTTPLCLPHHGLPSHSSVLSPSFQLQSHKNYEGTCEIPESKYSPLGGPKPFECPSIQITSISPNCHQELDAHEDDLQINDPEREFLERPSRDHLYLPLEPSYRESSLSPSPASSISSRSWFSDASSCESLSHIYDDVDSELNEAAARFTLGSPLTSPGGSPGGCPGEETWHQQYGLGHSLSPRQSPCHSPRSSVTDENWLSPRPASGPSSRPTSPCGKRRHSSAEVCYAGSLSPHHSPVPSPGHSPRGSVTEDTWLNASVHGGSGLGPAVFPFQYCVETDIPLKTRKTSEDQAAILPGKLELCSDDQGSLSPARETSIDDGLGSQYPLKKDSCGDQFLSVPSPFTWSKPKPGHTPIFRTSSLPPLDWPLPAHFGQCELKIEVQPKTHHRAHYETEGSRGAVKASTGGHPVVKLLGYNEKPINLQMFIGTADDRYLRPHAFYQVHRITGKTVATASQEIIIASTKVLEIPLLPENNMSASIDCAGILKLRNSDIELRKGETDIGRKNTRVRLVFRVHIPQPSGKVLSLQIASIPVECSQRSAQELPHIEKYSINSCSVNGGHEMVVTGSNFLPESKIIFLEKGQDGRPQWEVEGKIIREKCQGAHIVLEVPPYHNPAVTAAVQVHFYLCNGKRKKSQSQRFTYTPVLMKQEHREEIDLSSVPSLPVPHPAQTQRPSSDSGCSHDSVLSGQRSLICSIPQTYASMVTSSHLPQLQCRDESVSKEQHMIPSPIVHQPFQVTPTPPVGSSYQPMQTNVVYNGPTCLPINAASSQEFDSVLFQQDATLSGLVNLGCQPLSSIPFHSSNSGSTGHLLAHTPHSVHTLPHLQSMGYHCSNTGQRSLSSPVADQITGQPSSQLQPITYGPSHSGSATTASPAASHPLASSPLSGPPSPQLQPMPYQSPSSGTASSPSPATRMHSGQHSTQAQSTGQGGLSAPSSLICHSLCDPASFPPDGATVSIKPEPEDREPNFATIGLQDITLDDDQFISDLEHQPSGSAEKWPNHSVLSCPAPFWRI

SEQ ID NO: 170 - DNA, 3204 bp, Homo sapiens
  source 1..3204: mol_type = genomic DNA
atgactactgcaaactgtggcgcccacgacgagctcgacttcaaactcgtctttggcgaggacggggcgccggcgccgccgcccccgggctcgcggcctgcagatcttgagccagatgattgtgcatccatttacatctttaatgtagatccacctccatctactttaaccacaccactttgcttaccacatcatggattaccgtctcactcttctgttttgtcaccatcgtttcagctccaaagtcacaaaaactatgaaggaacttgtgagattcctgaatctaaatatagcccattaggtggtcccaaaccctttgagtgcccaagtattcaaattacatctatctctcctaactgtcatcaagaattagatgcacatgaagatgacctacagataaatgacccagaacgggaatttttggaaaggccttctagagatcatctctatcttcctcttgagccatcctaccgggagtcttctcttagtcctagtcctgccagcagcatctcttctaggagttggttctctgatgcatcttcttgtgaatcgctttcacatatttatgatgatgtggactcagagttgaatgaagctgcagcccgatttacccttggatcccctctgacttctcctggtggctctccagggggctgccctggagaagaaacttggcatcaacagtatggacttggacactcattatcacccaggcaatctccttgccactctcctagatccagtgtcactgatgagaattggctgagccccaggccagcctcaggaccctcatcaaggcccacatccccctgtgggaaacggaggcactccagtgctgaagtttgttatgctgggtccctttcaccccatcactcacctgttccttcacctggtcactcccccaggggaagtgtgacagaagatacgtggctcaatgcttctgtccatggtgggtcaggccttggccctgcagtttttccatttcagtactgtgtagagactgacatccctctcaaaacaaggaaaacttctgaagatcaagctgccatactaccaggaaaattagagctgtgttcagatgaccaagggagtttatcaccagcccgggagacttcaatagatgatggccttggatctcagtatcctttaaagaaagattcatgtggtgatcagtttctttcagttccttcaccctttacctggagcaaaccaaagcctggccacacccctatatttcgcacatcttcattacctccactagactggcctttaccagctcattttggacaatgtgaactgaaaatagaagtgcaacctaaaactcatcatcgagcccattatgaaactgaaggtagccgaggggcagtaaaagcatctactgggggacatcctgttgtgaagctcctgggctataacgaaaagccaataaatctacaaatgtttattgggacagcagatgatcgatatttacgacctcatgcattttaccaggtgcatcgaatcactgggaagacagtcgctactgcaagccaagagataataattgccagtacaaaagttctggaaattccacttcttcctgaaaataatatgtcagccagtattgattgtgcaggtattttgaaactccgcaattcagatatagaacttcgaaaaggagaaactgatattggcagaaagaatactagagtacgacttgtgtttcgtgtacacatcccacagcccagtggaaaagtcctttctctgcagatagcctctatacccgttgagtgctcccagcggtctgctcaagaacttcctcatattgagaagtacagtatcaacagttgttctgtaaatggaggtcatgaaatggttgtgactggatctaattttcttccagaatccaaaatcatttttcttgaaaaaggacaagatggacgacctcagtgggaggtagaagggaagataatcagggaaaaatgtcaaggggctcacattgtccttgaagttcctccatatcataacccagcagttacagctgcagtgcaggtgcacttttatctttgcaatggcaagaggaaaaaaagccagtctcaacgttttacttatacaccagttttgatgaagcaagaacacagagaagagattgatttgtcttcagttccatctttgcctgtgcctcatcctgctcagacccagaggccttcctctgattcagggtgttcacatgacagtgtactgtcaggacagagaagtttgatttgctccatcccacaaacatatgcatccatggtgacctcatcccatctgccacagttgcagtgtagagatgagagtgttagtaaagaacagcatatgattccttctccaattgtacaccagccttttcaagtcacaccaacacctcctgtggggtcttcctatcagcctatgcaaactaatgttgtgtacaatggaccaacttgtcttcctattaatgctgcctctagtcaagaatttgattcagttttgtttcagcaggatgcaactctttctggtttagtgaatcttggctgtcaaccactgtcatccataccatttcattcttcaaattcaggctcaacaggacatctcttagcccatacacctcattctgtgcataccctgcctcatctgcaatcaatgggatatcattgttcaaatacaggacaaagatctctttcttctccagtggctgaccagattacaggtcagccttcgtctcagttacaacctattacatatggtccttcacattcagggtctgctacaacagcttccccagcagcttctcatcccttggctagttcaccgctttctgggccaccatctcctcagcttcagcctatgccttaccaatctcctagctcaggaactgcctcatcaccgtctccagccaccagaatgcattctggacagcactcaactcaagcacaaagtacgggccaggggggtctttctgcaccttcatccttaatatgtcacagtttgtgtgatccagcgtcatttccacctgatggggcaactgtgagcattaaacctgaaccagaagatcgagagcctaactttgcaaccattggtctgcaggacatcactttagatgatgaccaatttatatctgacttggaacaccagccatcaggttcagcagagaaatggcctaaccacagtgtgctctcatgtccagctcctttctggagaatc

SEQ ID NO: 171 - protein, 964 aa, Homo sapiens
MITTLPSLLPASLASISHRVTNLPSNSLSHNPGLSKPDFPGNSSPGLPSSSSPGRDLGAPAGSMGAASCEDEELEFKLVFGEEKEAPPLGAGGLGEELDSEDAPPCCRLALGEPPPYGAAPIGIPRPPPPRPGMHSPPPRPAPSPGTWESQPARSVRLGGPGGGAGGAGGGRVLECPSIRITSISPTPEPPAALEDNPDAWGDGSPRDYPPPEGFGGYREAGGQGGGAFFSPSPGSSSLSSWSFFSDASDEAALYAACDEVESELNEAASRFGLGSPLPSPRASPRPWTPEDPWSLYGPSPGGRGPEDSWLLLSAPGPTPASPRPASPCGKRRYSSSGTPSSASPALSRRGSLGEEGSEPPPPPPLPLARDPGSPGPFDYVGAPPAESIPQKTRRTSSEQAVALPRSEEPASCNGKLPLGAEESVAPPGGSRKEVAGMDYLAVPSPLAWSKARIGGHSPIFRTSALPPLDWPLPSQYEQLELRIEVQPRAHHRAHYETEGSRGAVKAAPGGHPVVKLLGYSEKPLTLQMFIGTADERNLRPHAFYQVHRITGKMVATASYEAVVSGTKVLEMTLLPENNMAANIDCAGILKLRNSDIELRKGETDIGRKNTRVRLVFRVHVPQGGGKVVSVQAASVPIECSQRSAQELPQVEAYSPSACSVRGGEELVLTGSNFLPDSKVVFIERGPDGKLQWEEEATVNRLQSNEVTLTLTVPEYSNKRVSRPVQVYFYVSNGRRKRSPTQSFRFLPVICKEEPLPDSSLRGFPSASATPFGTDMDFSPPRPPYPSYPHEDPACETPYLSEGFGYGMPPLYPQTGPPPSYRPGLRMFPETRGTTGCAQPPAVSFLPRPFPSDPYGGRGSSFSLGLPFSPPAPFRPPPLPASPPLEGPFPSQSDVHPLPAEGYNKVGPGYGPGEGAPEQEKSRGGYSSGFRDSVPIQGITLEEGGCGTGGCECECVQEIALHVC

SEQ ID NO: 172 - DNA, 2892 bp, Homo sapiens
  source 1..2892: mol_type = genomic DNA
atgataaccaccctcccatctctcctacccgccagcctcgccagtatctcccaccgagtcacgaatctcccatctaactccctctcacacaacccaggcctctccaagcctgactttcccggaaactccagtccaggtcttccttcctcctccagcccaggccgggacctgggggctcctgccggatccatgggggcggccagctgcgaggatgaggagctggaatttaagctggtgttcggggaggaaaaggaggcccccccgctgggcgcggggggattgggggaagaactggactcagaggatgccccgccatgctgccgtctggccttgggagagccccctccctatggcgctgcacctatcggtattccccgacctccaccccctcggcctggcatgcattcgccaccgccgcgaccagccccctcacctggcacctgggagagccagcccgccaggtcggtgaggctgggaggaccaggagggggtgctgggggtgctgggggtggccgtgttctcgagtgtcccagcatccgcatcacctccatctctcccacgccggagccgccagcagcgctggaggacaaccctgatgcctggggggacggctctcctagagattaccccccaccagaaggctttgggggctacagagaagcagggggccagggtgggggggccttcttcagcccaagccctggcagcagcagcctgtcctcgtggagcttcttctccgatgcctctgacgaggcagccctgtatgcagcctgcgacgaggtggagtctgagctaaatgaggcggcctcccgctttggcctgggctccccgctgccctcgccccgggcctcccctcggccatggacccccgaagatccctggagcctgtatggtccaagccccggaggccgagggccagaggatagctggctactcctcagtgctcctgggcccaccccagcctccccgcggcctgcctctccatgtggcaagcggcgctattccagctcgggaaccccatcttcagcctccccagctctgtcccgccgtggcagcctgggggaagaggggtctgagccacctccaccacccccattgcctctggcccgggacccgggctcccctggtccctttgactatgtgggggccccaccagctgagagcatccctcagaagacacggcggacttccagcgagcaggcagtggctctgcctcggtctgaggagcctgcctcatgcaatgggaagctgcccttgggagcagaggagtctgtggctcctccaggaggttcccggaaggaggtggctggcatggactacctggcagtgccctccccactcgcttggtccaaggcccggattgggggacacagccctatcttcaggacctctgccctacccccactggactggcctctgcccagccaatatgagcagctggagctgaggatcgaggtacagcctagagcccaccaccgggcccactatgagacagaaggcagccgtggagctgtcaaagctgcccctggcggtcaccccgtagtcaagctcctaggctacagtgagaagccactgaccctacagatgttcatcggcactgcagatgaaaggaacctgcggcctcatgccttctatcaggtgcaccgtatcacaggcaagatggtggccacggccagctatgaagccgtagtcagtggcaccaaggtgttggagatgactctgctgcctgagaacaacatggcggccaacattgactgcgcgggaatcctgaagcttcggaattcagacattgagcttcggaagggtgagacggacatcgggcgcaaaaacacacgtgtacggctggtgttccgggtacacgtgccccagggcggcgggaaggtcgtctcagtacaggcagcatcggtgcccatcgagtgctcccagcgctcagcccaggagctgccccaggtggaggcctacagccccagtgcctgctctgtgagaggaggcgaggaactggtactgactggctccaacttcctgccagactccaaggtggtgttcattgagaggggtcctgatgggaagctgcaatgggaggaggaggccacagtgaaccgactgcagagcaacgaggtgacgctgaccctgactgtccccgagtacagcaacaagagggtttcccggccagtccaggtctacttttatgtctccaatgggcggaggaaacgcagtcctacccagagtttcaggtttctgcctgtgatctgcaaagaggagcccctaccggactcatctctgcggggtttcccttcagcatcggcaaccccctttggcactgacatggacttctcaccacccaggcccccctacccctcctatccccatgaagaccctgcttgcgaaactccttacctatcagaaggcttcggctatggcatgccccctctgtacccccagacggggcccccaccatcctacagaccgggcctgcggatgttccctgagactaggggtaccacaggttgtgcccaaccacctgcagtttccttccttccccgccccttccctagtgacccgtatggagggcggggctcctctttctccctggggctgccattctctccgccagccccctttcggccgcctcctcttcctgcatccccaccgcttgaaggccccttcccttcccagagtgatgtgcatcccctacctgctgagggatacaataaggtagggccaggctatggccctggggagggggctccggagcaggagaaatccaggggtggctacagcagcggcttccgagacagtgtccctatccagggtatcacgctggaggaaggtgggtgtgggactgggggctgtgagtgtgagtgtgtgcaagagattgctctgcatgtttgc

SEQ ID NO: 173 - protein, 485 aa, Homo sapiens
IQTVPDPAAHIKEALSVVSEDQSLFECAYGTPHLAKTEMTASSSSDYGQTSKMSPRVPQQDWLSQPPARVTIKMECNPSQVNGSRNSPDECSVAKGGKMVGSPDTVGMNYGSYMEEKHMPPPNMTTNERRVIVPADPTLWSTDHVRQWLEWAVKEYGLPDVNILLFQNIDGKELCKMTKDDFQRLTPSYNADILLSHLHYLRETPLPHLTSDDVDKALQNSPRLMHARNTGGAAFIFPNTSVYPEATQRITTRPDLPYEPPRRSAWTGHGHPTPQSKAAQPSPSTVPKTEDQRPQLDPYQILGPTSSRLANPGSGQIQLWQFLLELLSDSSNSSCITWEGTNGEFKMTDPDEVARRWGERKSKPNMNYDKLSRALRYYYDKNIMTKVHGKRYAYKFDFHGIAQALQPHPPESSLYKYPSDLPYMGSYHAHPQKMNFVAPHPPALPVTSSSFFAAPNPYWNSPTGGIYPNTRLPTSHMPSHLGTYY

SEQ ID NO: 174 - DNA, 1458 bp, Homo sapiens
  source 1..1458: mol_type = genomic DNA
atgattcagactgtcccggacccagcagctcatatcaaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaaggaactctcctgatgaatgcagtgtggccaaaggcgggaagatggtgggcagcccagacaccgttgggatgaactacggcagctacatggaggagaagcacatgccacccccaaacatgaccacgaacgagcgcagagttatcgtgccagcagatcctacgctatggagtacagaccatgtgcggcagtggctggagtgggcggtgaaagaatatggccttccagacgtcaacatcttgttattccagaacatcgatgggaaggaactgtgcaagatgaccaaggacgacttccagaggctcacccccagctacaacgccgacatccttctctcacatctccactacctcagagagactcctcttccacatttgacttcagatgatgttgataaagccttacaaaactctccacggttaatgcatgctagaaacacagggggtgcagcttttattttcccaaatacttcagtatatcctgaagctacgcaaagaattacaactaggccagatttaccatatgagccccccaggagatcagcctggaccggtcacggccaccccacgccccagtcgaaagctgctcaaccatctccttccacagtgcccaaaactgaagaccagcgtcctcagttagatccttatcagattcttggaccaacaagtagccgccttgcaaatccaggcagtggccagatccagctttggcagttcctcctggagctcctgtcggacagctccaactccagctgcatcacctgggaaggcaccaacggggagttcaagatgacggatcccgacgaggtggcccggcgctggggagagcggaagagcaaacccaacatgaactacgataagctcagccgcgccctccgttactactatgacaagaacatcatgaccaaggtccatgggaagcgctacgcctacaagttcgacttccacgggatcgcccaggccctccagccccaccccccggagtcatctctgtacaagtacccctcagacctcccgtacatgggctcctatcacgcccacccacagaagatgaactttgtggcgccccaccctccagccctccccgtgacatcttccagtttttttgctgccccaaacccatactggaattcaccaactgggggtatataccccaacactaggctccccaccagccatatgccttctcatctgggcacttactac

SEQ ID NO: 175 - protein, 798 aa, Homo sapiens [sequence omitted]

SEQ ID NO: 176 - DNA, 2394 bp, Homo sapiens
  source 1..2394: mol_type = genomic DNA
atggcgtgggacatgtgcaaccaggactctgagtctgtatggagtgacatcgagtgtgctgctctggttggtgaagaccagcctctttgcccagatcttcctgaacttgatctttctgaactagatgtgaacgacttggatacagacagctttctgggtggactcaagtggtgcagtgaccaatcagaaataatatccaatcagtacaacaatgagccttcaaacatatttgagaagatagatgaagagaatgaggcaaacttgctagcagtcctcacagagacactagacagtctccctgtggatgaagacggattgccctcatttgatgcgctgacagatggagacgtgaccactgacaatgaggctagtccttcctccatgcctgacggcacccctccaccccaggaggcagaagagccgtctctacttaagaagctcttactggcaccagccaacactcagctaagttataatgaatgcagtggtctcagtacccagaaccatgcaaatcacaatcacaggatcagaacaaaccctgcaattgttaagactgagaattcatggagcaataaagcgaagagtatttgtcaacagcaaaagccacaaagacgtccctgctcggagcttctcaaatatctgaccacaaacgatgaccctcctcacaccaaacccacagagaacagaaacagcagcagagacaaatgcacctccaaaaagaagtcccacacacagtcgcagtcacaacacttacaagccaaaccaacaactttatctcttcctctgaccccagagtcaccaaatgaccccaagggttccccatttgagaacaagactattgaacgcaccttaagtgtggaactctctggaactgcaggcctaactccacccaccactcctcctcataaagccaaccaagataacccttttagggcttctccaaagctgaagtcctcttgcaagactgtggtgccaccaccatcaaagaagcccaggtacagtgagtcttctggtacacaaggcaataactccaccaagaaagggccggagcaatccgagttgtatgcacaactcagcaagtcctcagtcctcactggtggacacgaggaaaggaagaccaagcggcccagtctgcggctgtttggtgaccatgactattgccagtcaattaattccaaaacggaaatactcattaatatatcacaggagctccaagactctagacaactagaaaataaagatgtctcctctgattggcaggggcagatttgttcttccacagattcagaccagtgctacctgagagagactttggaggcaagcaagcaggtctctccttgcagcacaagaaaacagctccaagaccaggaaatccgagccgagctgaacaagcacttcggtcatcccagtcaagctgtttttgacgacgaagcagacaagaccagtgaactgagggacagtgatttcagtaatgaacaattctccaaactacctatgtttataaattcaggactagccatggatggcctgtttgatgacagcgaagatgaaagtgataaactgagctacccttgggatggcacacaatcctattcattgttcaatgtgtctccttcttgttcttcttttaactctccatgtagagattctgtgtcaccacccaaatccttattttctcaaagaccccaaaggatgcgctctcgttcaaggtccttttctcgacacaggtcgtgttcccgatcaccatattccaggtcaagatcaaggtctccaggcagtagatcctcttcaagatcctgctattactatgagtcaagccactacagacaccgcacgcaccgaaattctcccttgtatgtgagatcacgttcaagatcgccctacagccgtcggcccaggtatgacagctacgaggaatatcagcacgagaggctgaagagggaagaatatcgcagagagtatgagaagcgagagtctgagagggccaagcaaagggagaggcagaggcagaaggcaattgaagagcgccgtgtgatttatgtcggtaaaatcagacctgacacaacacggacagaactgagggaccgttttgaagtttttggtgaaattgaggagtgcacagtaaatctgcgggatgatggagacagctatggtttcattacctaccgttatacctgtgatgcttttgctgctcttgaaaatggatacactttgcgcaggtcaaacgaaactgactttgagctgtacttttgtggacgcaagcaatttttcaagtctaactatgcagacctagattcaaactcagatgactttgaccctgcttccaccaagagcaagtatgactctctggattttgatagtttactgaaagaagctcagagaagcttgcgcagg

SEQ ID NO: 177 - protein, 431 aa, Homo sapiens
MASSLLEEEVHYGSSPLAMLTAACSKFGGSSPLRDSTTLGKAGTKKPYSVGSDLSASKTMGDAYPAPFTSTNGLLSPAGSPPAPTSGYANDYPPFSHSFPGPTGTQDPGLLVPKGHSSSDCLPSVYTSLDMTHPYGSWYKAGIHAGISPGPGNTPTPWWDMHPGGNWLGGGQGQGDGLQGTLPTGPAQPPLNPQLPTYPSDFAPLNPAPYPAPHLLQPGPQHVLPQDVYKPKAVGNSGQLEGSGGAKPPRGASTGGSGGYGGSGAGRSSCDCPNCQELERLGAAAAGLRKKPIHSCHIPGCGKVYGKASHLKAHLRWHTGERPFVCNWLFCGKRFTRSDELERHVRTHTREKKFTCLLCSKRFTRSDHLSKHQRTHGEPGPGPPPSGPKELGEGRSTGEEEASQTPRPSASPATPEKAPGGSPEQSNLLEI

SEQ ID NO: 178 - DNA, 1293 bp, Homo sapiens
  source 1..1293: mol_type = genomic DNA
atggcgtcctccctgcttgaggaggaagttcactatggctccagtcccctggccatgctgacggcagcgtgcagcaaatttggtggctctagccctctgcgggactcaacaactctgggcaaagcaggcacaaagaagccgtactctgtgggcagtgacctttcagcctccaaaaccatgggggatgcttatccagccccctttacaagcactaatgggctcctttcacctgcaggcagtcctccagcacccacctcaggctatgctaatgattaccctcccttttcccactcattccctgggcccacaggcacccaggaccctgggctactagtgcccaaggggcacagctcttctgactgtctgcccagtgtctacacctctctggacatgacacacccctatggctcctggtacaaggcaggcatccatgcaggcatttcaccaggcccaggcaacactcctactccatggtgggatatgcaccctggaggcaactggctaggtggtgggcagggccagggtgatgggctgcaagggacactgcccacaggtccagctcagcctccactgaacccccagctgcccacctacccatctgactttgctccccttaatccagccccctacccagctccccacctcttgcaaccagggccccagcatgtcttgccccaagatgtctataaacccaaggcagtgggaaatagtgggcagctagaagggagtggtggagccaaacccccacggggtgcaagcactgggggtagtggtggatatgggggcagtggggcagggcgctcctcctgcgactgccctaattgccaggagctagagcggctgggagcagcagcggctgggctgcggaagaagcccatccacagctgccacatccctggctgcggcaaggtgtatggcaaggcttcgcacctgaaggcccacttgcgctggcacacaggcgagaggcccttcgtctgcaactggctcttctgcggcaagaggttcactcgttcggatgagctggagcgtcatgtgcgcactcacacccgggagaagaagttcacctgcctgctctgctccaagcgctttacccgaagtgaccacctgagcaaacaccagcgcacccacggagaaccaggcccgggtccccctcccagtggccccaaggagctgggggagggccgcagcacgggggaagaggaggccagtcagacgccccgaccttctgcctcgccagcaaccccagagaaagcccctggaggcagccctgagcagagcaacttgctggagatc

SEQ ID NO: 179 - protein, 473 aa, Homo sapiens
MGRKKIQITRIMDERNRQVTFTKRKFGLMKKAYELSVLCDCEIALIIFNSTNKLFQYASTDMDKVLLKYTEYNEPHESRTNSDIVETLRKKGLNGCDSPDPDADDSVGHSPESEDKYRKINEDIDLMISRQRLCAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVSEDVDLLLNQRINNSQSAQSLATPVVSVATPTLPGQGMGGYPSAISTTYGTEYSLSSADLSSLSGFNTASALHLGSVTGWQQQHLHNMPPSALSQLGACTSTHLSQSSNLSLPSTQSLNIKSEPVSPPRDRTTTPSRYPQHTRHEAGRSPVDSLSSCSSSYDGSDREDHRNEFHSPIGLTRPSPDERESPSVKRMRLSEGWAT

SEQ ID NO: 180 - DNA, 1419 bp, Homo sapiens
  source 1..1419: mol_type = genomic DNA
atggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgtctgaggatgtcgacctgcttttgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagcttgcactagcactcatttatctcagagttcaaatctctccctgccttctactcaaagcctcaacatcaagtcagaacctgtttctcctcctagagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaaca

SEQ ID NO: 181 - protein, 352 aa, Homo sapiens
MNTIVFNKLSSAVLFEDGGASERERGGRPYSGVLDSPHARPEVGIPDGPPLKDNLGLRHRRTGARQNGGKVRHKRQALQDMARPLKQWLYKHRDNPYPTKTEKILLALGSQMTLVQVSNWFANARRRLKNTVRQPDLSWALRIKLYNKYVQGNAERLSVSSDDSCSEDGENPPRTHMNEGGYNTPVHHPVIKSENSVIKAGVRPESRASEDYVAPPKYKSSLLNRYLNDSLRHVMATNTTMMGKTRQRNHSGSFSSNEFEEELVSPSSSETEGNFVYRTDTLENGSNKGESAANRKGPSKDDTYWKEINAAMALTNLAQGKDKLQGTTSCIIQKSSHIAEVKTVKVPLVQQF

SEQ ID NO: 182 - DNA, 1056 bp, Homo sapiens
  source 1..1056: mol_type = genomic DNA
atgaacaccatcgtcttcaacaagctcagctctgcggtgctgtttgaggacggaggcgcctcggagcgggagcggggtggccggccctacagcggtgtcctggacagtcctcacgcccgccccgaggtgggcattcccgacggcccgcccctcaaggacaacctcggcctgagacaccggaggaccggcgcccggcagaatggcgggaaggtgaggcacaagcggcaggccctgcaagacatggcgcgacccctcaagcagtggctttacaagcaccgtgacaacccgtaccccaccaagaccgagaagatactcttggccctcggctcgcagatgacgctagtgcaggtgtcaaattggtttgctaatgcaagacgtcggcttaagaataccgttcgacagccagatttaagctgggctttgagaataaagttatacaacaagtatgttcaaggcaatgctgaacggcttagcgtaagcagtgatgactcatgttctgaagatggagaaaatcctccaagaacccacatgaacgaagggggctataataccccagttcaccatcctgtgattaaaagtgagaattcggtcatcaaagcgggagtgaggccagagtcacgggccagtgaggactacgtggcaccccccaaatacaagagcagcttgttgaaccgttaccttaatgactctttgagacatgtcatggccacgaacactaccatgatgggaaaaacaaggcaaagaaaccactcgggatcttttagctccaatgaatttgaggaagaattagtgtctccatcgtcatcagaaactgaaggcaactttgtctatcgcacagacactctggaaaatggatccaataagggtgaaagcgcagctaacagaaaaggaccaagcaaggatgacacgtattggaaggagatcaacgcagctatggccttaacaaatcttgcacagggaaaggacaaactgcagggaactaccagctgcatcatccagaagtcgtcccatatagcagaagtaaagactgtcaaagtgccgctggtgcagcagttt

SEQ ID NO: 183 - protein, 480 aa, Homo sapiens
MSFPQLGYPQYLSAAGPGAYGGERPGVLAAAAAAAAAASSGRPGAAELGGGAGAAAVTSVLGMYAAAGPYAGAPNYSAFLPYAADLSLFSQMGSQYELKDNPGVHPATFAAHTAPAYYPYGQFQYGDPGRPKNATRESTSTLKAWLNEHRKNPYPTKGEKIMLAIITKMTLTQVSTWFANARRRLKKENKVTWGARSKDQEDGALFGSDTEGDPEKAEDDEEIDLESIDIDKIDEHDGDQSNEDDEDKAEAPHAPAAPSALARDQGSPLAAADVLKPQDSPLGLAKEAPEPGSTRLLSPGAAAGGLQGAPHGKPKIWSLAETATSPDGAPKASPPPPAGHPGAHGPSAGAPLQHPAFLPSHGLYTCHIGKFSNWTNSAFLAQGSLLNMRSFLGVGAPHAAPHGPHLPAPPPPQPPVAIAPGALNGDKASVRSSPTLPERDLVPRPDSPAQQLKSPFQPVRDNSLAPQEGTPRILAALPSA

SEQ ID NO: 184 - DNA, 1440 bp, Homo sapiens
  source 1..1440: mol_type = genomic DNA
atgtccttcccgcagctgggctacccgcagtacctgagcgccgcggggccgggcgcctacggcggcgagcgcccgggggtgctggccgcggccgctgcggcggctgccgccgcctcgtcgggccgaccgggggccgcggagctgggcggcggggcaggcgcggctgcagtcacctcggtgctgggcatgtacgcggcggcggggccgtacgcgggcgcgcccaactacagcgccttcctgccctacgccgcggatctcagcctcttctcgcagatgggctcgcagtatgaactgaaggacaaccctggggtgcaccccgccaccttcgcagcccacacggcgccggcttattacccctacggccagttccaatacggggaccccgggcggcccaagaacgccacccgcgagagcaccagcacgctcaaggcctggctcaacgagcaccgcaagaatccctaccccaccaagggcgagaagatcatgctggccatcatcaccaagatgaccctcacgcaggtctccacctggttcgccaacgcgcgccggcgcctcaagaaggagaacaaggtgacatggggagcgcgcagcaaggaccaggaagatggagcgctcttcggcagcgacaccgagggcgacccggagaaggccgaggacgacgaggagatcgacctggaaagcatcgacattgacaagatcgacgagcacgatggcgaccagagcaacgaggatgacgaggacaaggccgaggctccgcacgcgcccgcagccccttctgctcttgcccgggaccaaggctcgccgctggcagcagccgacgttctcaagccccaggactcgcccttgggcctggcaaaggaggccccagagccgggcagcacgcgcctgctgagccccggcgctgcagcgggcggcctgcagggtgcgccgcacggcaagcccaagatctggtcgctggcggagacagccacgagccccgacggtgcgcccaaggcttcgccaccaccacccgcgggccaccccggcgcgcacgggccctccgccggggcgccgctgcaacaccccgccttcctgcctagccacggactgtacacctgccacatcggcaagttctccaactggaccaacagcgcattcctcgcacagggctccctgctcaacatgcgctccttcctgggcgttggcgctccccacgccgcgccccatggccctcaccttcctgcacctccaccaccgcagccgccggtcgctattgccccgggggcactcaatggagacaaggcctcggtccgcagcagccccacgctcccagagagagacctcgtccccaggccagattcgccggcacagcagttaaagtcgcccttccagccggtacgcgacaactctctggccccgcaggagggaacgccgcggatcctagcagccctcccgtccgcc

SEQ ID NO: 185 - protein, 471 aa, Homo sapiens
MSYPQGYLYQAPGSLALYSCPAYGASALAAPRSEELARSASGSAFSPYPGSAAFTAQAATGFGSPLQYSADAAAAAAGFPSYMGAPYDAHTTGMTGAISYHPYGSAAYPYQLNDPAYRKNATRDATATLKAWLNEHRKNPYPTKGEKIMLAIITKMTLTQVSTWFANARRRLKKENKMTWAPRNKSEDEDEDEGDATRSKDESPDKAQEGTETSAEDEGISLHVDSLTDHSCSAESDGEKLPCRAGDPLCESGSECKDKYDDLEDDEDDDEEGERGLAPPKPVTSSPLTGLEAPLLSPPPEAAPRGGRKTPQGSRTSPGAPPPASKPKLWSLAEIATSDLKQPSLGPGCGPPGLPAAAAPASTGAPPGGSPYPASPLLGRPLYYTSPFYGNYTNYGNLNAALQGQGLLRYNSAAAAPGEALHTAPKAASDAGKAGAHPLESHYRSPGGGYEPKKDASEGCTVVGGGVQPYL

SEQ ID NO: 186 - DNA, 1413 bp, Homo sapiens
  source 1..1413: mol_type = genomic DNA
atgtcctacccgcagggctacctgtaccaggcgcccggctcgctggcgctctactcgtgcccggcctacggcgcgtcggctttggcggctccgcgcagcgaggagctggcgcgctcggcgtcgggctcggcgttcagcccctacccgggctcggcggccttcacggcgcaggcggccaccggcttcgggagcccgctgcagtactcggccgacgccgccgccgccgccgccggcttcccgtcctacatgggcgcaccctacgacgcgcacaccaccggcatgaccggcgccatcagctaccacccgtacggcagcgcggcctacccgtaccagctcaacgaccccgcgtaccgcaagaacgccacgcgggacgccacggccactctcaaggcctggctcaacgagcaccgcaagaacccctaccccaccaagggcgagaagatcatgctagccatcatcaccaagatgaccctcacccaggtctccacctggttcgccaacgcgcgccggcgcctcaagaaggagaacaagatgacctgggccccgagaaacaaaagcgaagatgaggacgaggacgagggcgacgctaccagaagcaaggacgagagtcccgacaaggcgcaggagggcacggagacctcggcagaggacgaagggatcagcctgcacgtggactcgctcacggatcactcgtgctcggccgagtcggacggggagaagcttccgtgccgcgccggggaccccctgtgcgaatcgggctcggagtgcaaggacaagtatgacgacctggaggacgacgaggacgacgacgaggagggcgagcggggcctggcgccgcccaagcccgtgacctcgtcgccgcttaccggcttggaggcgccgctgctgagccccccgcccgaggccgcgccccgcggtggccgcaagacgccccagggcagccggacgtctccgggcgcgccgccccccgccagcaagcccaagctgtggtcgctggccgagatcgccacgtcggacctcaagcagccgagcctgggcccgggctgcgggccaccggggctgcccgcggccgccgcgccggcctcaaccggggcaccgccaggaggctcgccctaccctgcctcgccgctgctgggccgccccctctactacacgtcgcccttctacggcaactacacaaactacgggaacttgaacgcggcgctgcagggccagggtctcctgcggtacaactctgcggccgcggcccccggcgaggccctgcacaccgcgccaaaggcggccagcgacgcgggcaaggcgggcgcgcacccgctcgagtcccactaccggtccccgggcggcggctacgagcccaagaaagatgccagcgagggctgcaccgtggttggcgggggcgtccagccctaccta

SEQ ID NO: 187 - protein, 501 aa, Homo sapiens [sequence omitted]

SEQ ID NO: 188 - DNA, 1503 bp, Homo sapiens
  source 1..1503: mol_type = genomic DNA
atgtccttcccccagctgggataccaatacatccgcccgctttacccgtccgagcgcccgggggccgctggcggcagcggcggcagcgcgggggcccggggcggcctgggtgccggagcctcggagctgaacgcctcggggtccctgtccaacgtgctctcgtccgtgtacggggcgccctacgccgcggccgctgcggccgccgccgcccaaggctacggcgccttcctgccctacgccgcggagctgcccatcttcccgcagctgggcgcgcagtatgagctgaaggacagccccggggtgcagcatccggccgcggctgccgcgtttccgcacccgcaccccgccttctacccgtatggccagtaccagttcggggacccgtcccgtcccaagaacgccaccagggagagcaccagcacgctgaaggcctggctcaacgagcaccgcaagaacccctaccccaccaagggcgagaagatcatgctggccatcatcaccaagatgaccctcacccaggtgtccacctggttcgccaacgcgcgccggcgcctcaagaaggagaataagatgacttgggcgcctcgcagccgcactgacgaggagggaaacgcttatgggagcgagcgcgaggaggaagacgaagaggaggacgaggaggacggcaaacgcgagctagagctggaggaggaggagctcgggggggaggaggaggacacggggggcgagggcctggctgacgacgacgaggacgaggagatcgatttggagaacttagacggcgcggccaccgagcctgagctgtccctggctggggcggcgcgcagggatggcgacctaggcctgggacccatttcggactccaaaaatagcgactcggaagatagctctgagggcttagaggaccggccactaccggtcctgagtctggctccagcgccaccaccagtggccgtggcctcgccgtctctgccgtcgccccccgtgagcctggacccctgcgctcccgcaccagcccccgcctccgccctgcagaagcccaagatctggtccctcgcggagactgccacaagcccggacaacccgcgccgctcgcctcccggcgcgggggggtctccaccgggggcagcggtcgcgccttccgccctgcagctctctccggccgccgccgccgccgccgctcacagactggtctcagcgccgctgggcaagttcccggcttggaccaaccggccgtttccaggcccaccgcccggcccccgcccgcacccgctctccctgctgggctctgcccctccgcacctgctgggacttcccggagccgcgggccacccggctgccgccgccgccttcgctcggccagcggagcccgaaggcggaacagatcgctgtagtgccttggaagtggagaaaaagttactcaagacagctttccagcccgtgcccaggcggccccagaaccatctggacgccgccctggtcttatcggctctctcctcatcc

SEQ ID NO: 189 - protein, 519 aa, Homo sapiens
MSYPQFGYPYSSAPQFLMATNSLSTCCESGGRTLADSGPAASAQAPVYCPVYESRLLATARHELNSAAALGVYGGPYGGSQGYGNYVTYGSEASAFYSLNSFDSKDGSGSAHGGLAPATAAYYPYEPALGQYPYDRYGTMDSGTRRKNATRETTSTLKAWLQEHRKNPYPTKGEKIMLAIITKMTLTQVSTWFANARRRLKKENKMTWPPRNKCADEKRPYAEGEEEEGGEEEAREEPLKSSKNAEPVGKEEKELELSDLDDFDPLEAEPPACELKPPFHSLDGGLERVPAAPDGPVKEASGALRMSLAAGGGAALDEDLERARSCLRSAAAGPEPLPGAEGGPQVCEAKLGFVPAGASAGLEAKPRIWSLAHTATAAAAAATSLSQTEFPSCMLKRQGPAAPAAVSSAPATSPSVALPHSGALDRHQDSPVTSLRNWVDGVFHDPILRHSTLNQAWATAKGALLDPGPLGRSLGAGANVLTAPLARAFPPAVPQDAPAAGAARELLALPKAGGKPFCA

SEQ ID NO: 190 - DNA, 1557 bp, Homo sapiens
  source 1..1557: mol_type = genomic DNA
atgtcctacccgcagtttggatacccctactcctcggctccccagttcttgatggccaccaactccctgagcacgtgctgcgagtccggaggccgcacgctggcggactccgggcccgccgcctcggcccaggcgccggtctactgcccggtctacgagagccggctgctggccaccgcgcgccacgagctcaactcggccgcggcgctgggcgtctatgggggtccctatggcggatcgcagggctatggcaactacgtgacctacggctcggaggcgtccgccttctactcgctgaacagctttgattccaaggatggttcgggatctgcgcatgggggcctggcaccagccactgccgcctactacccttacgagccagctctgggccagtacccctatgacaggtatggaaccatggacagcggcacgcggcgcaagaacgccacgcgcgagaccaccagcacgctcaaggcctggctgcaggagcaccgcaagaacccctaccccaccaagggcgagaagatcatgctggccatcatcaccaagatgaccctcacacaggtctccacctggttcgccaacgcgcgccggcgcctcaagaaggagaacaagatgacgtggccgccgcggaacaagtgcgcagacgagaagcggccctacgcggagggcgaggaggaggaggggggcgaggaggaggcgcgggaggagcccctcaagagctccaagaacgcagagcccgtgggcaaagaggagaaggagctggagcttagtgacttggacgacttcgacccgctggaagcagagccgccggcgtgcgagctgaagccgcccttccactccctggacggcggtctggagcgcgtccccgccgcgcccgacggcccggtcaaggaggcctcaggcgcgctccggatgtctctggccgcgggtggcggagctgctctggacgaggacctggagagggcccggagctgtctccgcagcgcggcggccgggccggagccactgccgggcgcagagggcggccctcaggtctgcgaggccaagctggggtttgtgccggcgggggcgtcggcaggcctggaggctaagccgcgcatctggtccctggcccacacagccaccgccgccgccgccgccgccacctccctgagccagactgagtttccgtcgtgcatgctcaagcgccaaggtcccgcggcccctgcggctgtgtcctccgcgcccgccacgtccccgtctgtggcccttccccactctggcgccctggacaggcaccaggactccccggtaaccagtctcagaaactgggtggacggggtcttccacgaccccatcctcaggcacagcactttgaaccaggcctgggccaccgccaagggcgccctcctggaccccgggcctctgggacgctcgctgggggcgggcgcgaacgtgctgactgcacccctggcccgcgcctttccgcctgccgtgccccaggacgccccagctgcaggcgccgccagggagctgctcgccctgcccaaggccggcggcaaacccttctgcgcc

SEQ ID NO: 191 - protein, 483 aa, Homo sapiens
MSYPQGYLYQPSASLALYSCPAYSTSVISGPRTDELGRSSSGSAFSPYAGSTAFTAPSPGYNSHLQYGADPAAAAAAAFSSYVGSPYDHTPGMAGSLGYHPYAAPLGSYPYGDPAYRKNATRDATATLKAWLNEHRKNPYPTKGEKIMLAIITKMTLTQVSTWFANARRRLKKENKMTWTPRNRSEDEEEEENIDLEKNDEDEPQKPEDKGDPEGPEAGGAEQKAASGCERLQGPPTPAGKETEGSLSDSDFKEPPSEGRLDALQGPPRTGGPSPAGPAAARLAEDPAPHYPAGAPAPGPHPAAGEVPPGPGGPSVIHSPPPPPPPAVLAKPKLWSLAEIATSSDKVKDGGGGNEGSPCPPCPGPIAGQALGGSRASPAPAPSRSPSAQCPFPGGTVLSRPLYYTAPFYPGYTNYGSFGHLHGHPGPGPGPTTGPGSHFNGLNQTVLNRADALAKDPKMLRSQSQLDLCKDSPYELKKGMSDI

SEQ ID NO: 192 - DNA, 1449 bp, Homo sapiens
  source 1..1449: mol_type = genomic DNA
atgtcctatccgcagggctacttgtaccagccgtccgcctcgctggcgctctactcgtgcccggcgtacagcaccagcgtcatttcggggccccgcacggatgagctcggccgctcttcttcgggctccgcgttctcgccctacgctggctcgactgccttcacggcgccctcgccgggctacaactcgcacctccagtacggcgccgaccccgcggccgccgccgccgccgccttctcctcgtacgtgggctctccctacgaccacacacccggcatggcgggctccttggggtaccatccttacgcggcgcccctgggatcgtacccttacggggacccagcgtaccggaagaacgccacaagggacgccacggctaccctcaaggcctggctcaacgagcaccgcaagaacccctaccccaccaagggcgagaagatcatgctggccatcatcaccaagatgaccctcacccaggtgtccacctggttcgccaacgcgcgccggcgcctcaagaaagagaataaaatgacgtggacgccgcggaaccgcagcgaggacgaggaagaggaggagaacattgacctggagaagaacgacgaggacgagccccagaagcccgaggacaagggcgaccccgagggccccgaagcaggaggagctgagcagaaggcggcttcgggctgcgaacggcttcagggaccacccacccctgcaggcaaggagacggagggcagcctcagcgactcggattttaaggagccgccctcggagggccgcctcgacgcgctgcagggccccccccgcaccggcgggccctccccggctgggccagcggcggcgcggctggcggaggacccggcccctcactaccccgccggagcgccggcgcccggcccgcatccagccgcgggcgaggtgcctccgggtcccggcgggccctcggttatccattcgccgcctccgccgccgcctcctgcggtgctcgccaagcccaaactgtggtctttggcagagatcgccacatcgtcggacaaggtcaaggacgggggcggcgggaacgagggctctccatgcccaccgtgtcccgggcccatagccgggcaagccctaggaggcagccgggcgtcgccggccccggcgccgtcacgctcgccctcggcgcagtgtccttttccaggcgggacggtgctgtcccggcctctctactacaccgcgcccttctatcccggctacacgaactatggctccttcggacaccttcatggccacccggggcccgggccaggccccacaaccggtccggggtctcatttcaatggattaaaccagaccgtgttgaaccgagcggacgctttggctaaagacccgaaaatgttgcggagccagtctcagctagacctgtgcaaagactctccctatgaattgaagaaaggtatgtccgacatt

SEQ ID NO: 193 - protein, 446 aa, Homo sapiens
MSFPHFGHPYRGASQFLASASSSTTCCESTQRSVSDVASGSTPAPALCCAPYDSRLLGSARPELGAALGIYGAPYAAAAAAQSYPGYLPYSPEPPSLYGALNPQYEFKEAAGSFTSSLAQPGAYYPYERTLGQYQYERYGAVELSGAGRRKNATRETTSTLKAWLNEHRKNPYPTKGEKIMLAIITKMTLTQVSTWFANARRRLKKENKMTWAPKNKGGEERKAEGGEEDSLGCLTADTKEVTASQEARGLRLSDLEDLEEEEEEEEEAEDEEVVATAGDRLTEFRKGAQSLPGPCAAAREGRLERRECGLAAPRFSFNDPSGSEEADFLSAETGSPRLTMHYPCLEKPRIWSLAHTATASAVEGAPPARPRPRSPECRMIPGQPPASARRLSVPRDSACDESSCIPKAFGNPKFALQGLPLNCAPCPRRSEPVVQCQYPSGAEAG

SEQ ID NO: 194 - DNA, 1338 bp, Homo sapiens
  source 1..1338: mol_type = genomic DNA
atgtccttcccacactttggacacccgtaccgcggcgcttcccagtttctggcgtcggcaagttccagcaccacatgctgcgaatctacccaacgctctgtctcagatgtggcatcaggctccaccccagcgcccgctctctgctgcgcaccctacgatagtcgactgctgggcagtgcgcgaccggagctgggcgccgccttgggcatctatggagcaccctatgcggccgctgcagctgcccagagctaccctggctacctgccctatagcccagagcccccctcactgtatggggcactgaatccacagtatgaatttaaggaggctgcagggagttttacatccagcctggcacaaccaggagcctattatccctatgagcggactctggggcagtaccaatatgaacggtatggcgcagtggaattgagtggcgccggtcgccgaaagaacgcgacccgggagaccaccagtacactcaaggcctggctcaacgagcaccgcaaaaacccctaccccactaagggtgagaagatcatgctggccatcatcaccaagatgaccctcacccaggtgtccacctggttcgccaacgcacgccggcgcctcaagaaagagaacaaaatgacatgggcgcccaagaacaaaggtggggaggagaggaaggcagagggaggagaggaggactcactaggctgcctaactgctgacaccaaagaagttactgctagccaggaggcccgggggctccggctgagtgacctggaagacctggaggaagaggaggaggaggaggaggaagctgaagacgaggaggtagtggccacagctggggacaggctgacggagttccgaaagggcgcgcagtcactgcctgggccgtgcgctgcagctcgagagggccgattggagcgcagggagtgcggcctggctgcgccccgcttctccttcaatgacccttccggatcggaagaagctgacttcctctcggcggagacaggcagccctaggttgaccatgcactacccatgcttggagaaaccgcgcatctggtctctggcgcacaccgcgacagccagcgctgttgaaggtgcacccccagcccggcctaggccacgaagtcctgagtgccgtatgattcctggacagcctcctgcctctgcccggcgactctcagtccccagagactccgcgtgcgacgagtcttcctgcatacccaaagcctttggaaaccccaagtttgccctgcagggactaccgctgaactgtgcgccgtgcccgcggaggagcgagcctgtagtgcagtgccagtacccgtctggagcagaagcaggt

SEQ ID NO: 195 - protein, 201 aa, Homo sapiens
MSFATLRPAPPGRYLYPEVSPLSEDEDRGSDSSGSDEKPCRVHAARCGLQGARRRAGGRRAGGGGPGGRPGREPRQRHTANARERDRTNSVNTAFTALRTLIPTEPADRKLSKIETLRLASSYISHLGNVLLAGEACGDGQPCHSGPAFFHAARAGSPPPPPPPPPARDGENTQPKQICTFCLSNQRKLSKDRDRKTAIRS

SEQ ID NO: 196 - DNA, 603 bp, Homo sapiens
  source 1..603: mol_type = genomic DNA
atgtccttcgccacgctgcgcccggcgccgccgggccgctacctgtaccccgaggtgagcccgctgtcggaggacgaggaccgcggcagcgacagctcgggctccgacgagaaaccctgtcgcgtgcacgcggcgcgctgcggcctccagggcgcccggcggagggcggggggccggcgggccgggggcggggggccagggggccggccaggccgtgagccccggcagcggcacacggcgaacgcgcgcgagcgagaccgcaccaacagcgtgaacacggccttcacggcgctgcgcacgctgatccccaccgagcccgccgaccgcaagctctccaagattgagacgctgcgcctggcctccagctacatctcgcacctgggcaacgtgctgctggcgggcgaggcctgcggcgacggacagccctgccactccgggcccgccttcttccacgcggcgcgcgccggcagccccccgccgccgcccccgccgcctcccgcccgcgacggcgagaacacccagcccaaacagatctgcaccttctgcctcagcaaccagagaaagttgagcaaggaccgcgacagaaagacagcgattcgcagt

SEQ ID NO: 197 - protein, 480 aa, Homo sapiens
MASDSIFESFPSYPQCFMRECILGMNPSRDVHDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 198 - DNA, 1440 bp, Homo sapiens
  source 1..1440: mol_type = genomic DNA
atggcttcagacagcatatttgagtcatttccttcgtacccacagtgcttcatgagagaatgcatacttggaatgaatccttctagagacgtccacgatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctac

SEQ ID NO: 199 - protein, 589 aa, Homo sapiens
MLHSPHKQPQNHKCGANFLQEDSKKSLVFKWLISAGHYQPPRPTESFKAASSIYNRGYKFYLKKKGGTMASNSLFSTVTPCQQNFFWDPSTSRRFSPPSSSLQPGKMSDVSPVVAAQQQQQQQQQQQQQQQQQQQQQQQEAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSTLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSGSYQFPMVPGGDRSPSRMLPPCTTTSNGSTLLNPNLPNQNDGVDADGSHSSSPTVLNSSGRMDESVWRPY

SEQ ID NO: 200 - DNA, 1767 bp, Homo sapiens
  source 1..1767: mol_type = genomic DNA
atgcttcattcgcctcacaaacaaccacagaaccacaagtgcggtgcaaactttctccaggaggacagcaagaagtctctggtttttaaatggttaatctccgcaggtcactaccagccaccgagaccaacagagtcatttaaggctgcaagcagtatttacaacagagggtacaagttctatctgaaaaaaaaaggagggactatggcatcaaacagcctcttcagcacagtgacaccatgtcagcaaaacttcttttgggatccgagcaccagccggcgcttcagccccccctccagcagcctgcagcccggcaaaatgagcgacgtgagcccggtggtggctgcgcaacagcagcagcaacagcagcagcagcaacagcagcagcagcagcagcaacagcagcagcagcagcaggaggcggcggcggcggctgcggcggcggcggcggctgcggcggcggcagctgcagtgccccggttgcggccgccccacgacaaccgcaccatggtggagatcatcgccgaccacccggccgaactcgtccgcaccgacagccccaacttcctgtgctcggtgctgccctcgcactggcgctgcaacaagaccctgcccgtggccttcaaggtggtagccctcggagaggtaccagatgggactgtggttactgtcatggcgggtaacgatgaaaattattctgctgagctccggaatgcctctgctgttatgaaaaaccaagtagcaaggttcaacgatctgagatttgtgggccggagtggacgaggcaagagtttcaccttgaccataaccgtcttcacaaatcctccccaagtagctacctatcacagagcaattaaagttacagtagatggacctcgggaacccagaaggcacagacagaagcttgatgactctaaacctagtttgttctctgaccgcctcagtgatttagggcgcattcctcatcccagtatgagagtaggtgtcccgcctcagaacccacggccctccctgaactctgcaccaagtccttttaatccacaaggacagagtcagattacagaccccaggcaggcacagtcttccccgccgtggtcctatgaccagtcttacccctcctacctgagccagatgacgtccccgtccatccactctaccaccccgctgtcttccacacggggcactgggcttcctgccatcaccgatgtgcctaggcgcatttcagatgatgacactgccacctctgacttctgcctctggccttccactctcagtaagaagagccaggcaggtgcttcagaactgggccctttttcagaccccaggcagttcccaagcatttcatccctcactgagagccgcttctccaacccacgaatgcactatccagccacctttacttacaccccgccagtcacctcaggcatgtccctcggtatgtccgccaccactcactaccacacctacctgccaccaccctaccccggctcttcccaaagccagagtggacccttccagaccagcagcactccatatctctactatggcacttcgtcaggatcctatcagtttcccatggtgccggggggagaccggtctccttccagaatgcttccgccatgcaccaccacctcgaatggcagcacgctattaaatccaaatttgcctaaccagaatgatggtgttgacgctgatggaagccacagcagttccccaactgttttgaattctagtggcagaatggatgaatctgtttggcgaccatat

SEQ ID NO: 201 - protein, 429 aa, Homo sapiens
MASNSIFDSFPTYSPTFIRDPSTSRRFTPPSPAFPCGGGGGKMGENSGALSAQAAVGPGGRARPEVRSMVDVLADHAGELVRTDSPNFLCSVLPSHWRCNKTLPVAFKVVALGDVPDGTVVTVMAGNDENYSAELRNASAVMKNQVARFNDLRFVGRSGRGKSFTLTITVFTNPTQVATYHRAIKVTVDGPREPRRHRQKLEDQTKPFPDRFGDLERLRMRVTPSTPSPRGSLSTTSHFSSQPQTPIQGTSELNPFSDPRQFDRSFPTLPTLTESRFPDPRMHYPGAMSAAFPYSATPSGTSISSLSVAGMPATSRFHHTYLPPPYPGAPQNQSGPFQANPSPYHLYYGTSSGSYQFSMVAGSSSGGDRSPTRMLASCTSSAASVAAGNLMNPSLGGQSDGVEADGSHSNSPTALSTPGRMDEAVWRPY

SEQ ID NO: 202 - DNA, 1287 bp, Homo sapiens
  source 1..1287: mol_type = genomic DNA
atggcatcgaacagcatcttcgactccttcccgacctactcgccgaccttcatccgcgacccaagcaccagccgccgcttcacacctccctccccggccttcccctgcggcggcggcggcggcaagatgggcgagaacagcggcgcgctgagcgcgcaggcggccgtggggcccggagggcgcgcccggcccgaggtgcgctcgatggtggacgtgctggcggaccacgcaggcgagctcgtgcgcaccgacagccccaacttcctctgctccgtgctgccctcgcactggcgctgcaacaagacgctgcccgtcgccttcaaggtggtggcattgggggacgtgccggatggtacggtggtgactgtgatggcaggcaatgacgagaactactccgctgagctgcgcaatgcctcggccgtcatgaagaaccaggtggccaggttcaacgaccttcgcttcgtgggccgcagtgggcgagggaagagtttcaccctgaccatcactgtgttcaccaaccccacccaagtggcgacctaccaccgagccatcaaggtgaccgtggacggaccccgggagcccagacggcaccggcagaagctggaggaccagaccaagccgttccctgaccgctttggggacctggaacggctgcgcatgcgggtgacaccgagcacacccagcccccgaggctcactcagcaccacaagccacttcagcagccagccccagaccccaatccaaggcacctcggaactgaacccattctccgacccccgccagtttgaccgctccttccccacgctgccaaccctcacggagagccgcttcccagaccccaggatgcattatcccggggccatgtcagctgccttcccctacagcgccacgccctcgggcacgagcatcagcagcctcagcgtggcgggcatgccggccaccagccgcttccaccatacctacctcccgccaccctacccgggggccccgcagaaccagagcgggcccttccaggccaacccgtccccctaccacctctactacgggacatcctctggctcctaccagttctccatggtggccggcagcagcagtgggggcgaccgctcacctacccgcatgctggcctcttgcaccagcagcgctgcctctgtcgccgccggcaacctcatgaaccccagcctgggcggccagagtgatggcgtggaggccgacggcagccacagcaactcacccacggccctgagcacgccaggccgcatggatgaggccgtgtggcggccctac

SEQ ID NO: 203 - protein, 341 aa, Homo sapiens
MEPAFGEVNQLGGVFVNGRPLPNAIRLRIVELAQLGIRPCDISRQLRVSHGCVSKILARYNETGSILPGAIGGSKPRVTTPTVVKHIRTYKQRDPGIFAWEIRDRLLADGVCDKYNVPSVSSISRILRNKIGNLAQQGHYDSYKQHQPTPQPALPYNHIYSYPSPITAAAAKVPTPPGVPAIPGSVAMPRTWPSSHSVTDILGIRSITDQVSDSSPYHSPKVEEWSSLGRNNFPAAAPHAVNGLEKGALEQEAKYGQAPNGLPAVGSFVSASSMAPYPTPAQVSPYMTYSAAPSGYVAGHGWQHAGGTSLSPHNCDIPASLAFKGMQAAREGSHSVTASAL

SEQ ID NO: 204 - DNA, 4166 bp, Homo sapiens
  source 1..4166: mol_type = genomic DNA
agcccagcccacgttgctgcttagattgaaatgcagaactcaagcctctttcatcggggcacagacttccttttacttcttccttttgccctctcgcctcctcctcctgggaagaagcggaggcgccggcggtcggccgggatagcaacaggccgggccactgaggcggtgcggaaagtttctgtctgggagtgcggaactggggccgggttggtgtactgctcggagcaatggagccagccttcggggaggtgaaccagctgggaggagtgttcgtgaacgggaggccgctgcccaacgccatccggcttcgcatcgtggaactggcccaactgggcatccgaccgtgtgacatcagccgccagctacgggtctcgcacggctgcgtcagcaagatcctggcgcgatacaacgagacgggctcgatcttgccaggagccatcgggggcagcaagccccgggtcactacccccaccgtggtgaaacacatccggacctacaagcagagagaccccggcatcttcgcctgggagatccgggaccgcctgctggcggacggcgtgtgcgacaagtacaatgtgccctccgtgagctccatcagccgcattctgcgcaacaagatcggcaacttggcccagcagggtcattacgactcatacaagcagcaccagccgacgccgcagccagcgctgccctacaaccacatctactcgtaccccagccctatcacggcggcggccgccaaggtgcccacgccacccggggtgcctgccatccccggttcggtggccatgccgcgcacctggccctcctcgcactccgtcaccgacatcctgggcatccgctccatcaccgaccaagtgagcgacagctccccctaccacagccccaaggtggaggagtggagcagcctgggccgcaacaacttccccgccgccgccccgcacgcggtgaacgggttggagaagggagccctggagcaggaagccaagtacggtcaggcaccaaatggtctcccagctgtgggcagttttgtgtcagcatccagcatggctccttaccctaccccagcccaagtgtcgccttacatgacctacagtgctgctccttctggttatgttgctggacatgggtggcaacatgctgggggcacctcattgtctccccacaactgtgacattccggcatcgctggcgttcaagggaatgcaggcagccagagaaggtagtcattctgtcacggcttccgcgctctgatgggaaattccgtctccagcagcttcacccgggtctccctgtctcagcacctcctcccccaattcccaggtctcacatcccacccctcctgccctccaacccttctgccttgaaagctggctgtacggactcacatcctttgtgctaatgacacttacatatttcttgccataacttttctcttgcagaaaaactgacatgactttaggatttaaaaacaagagcaacaataagcattgaatgagacatttgtgttgcccacatactgtcttaacataacaaagaaacctacacccctcaaagggtttaaggaactttacaaactagtctttggtaaaaccacatgtgtatatttattctaaatcaacctgaacttttgaaatgtgcaattgttgagattttgcaaaatcaataaaggaaaatacttatagaaaaaattatgctacaccctctaatcaaatatggtaaccaagtaagctttaattcatcattaggaaacaaatcaataagtgacttgtttgagtgatcctttgtttaagacatgacctattttgttgaaaaatatatgtagaacccaagcaatatctgaatctagctctccctggtgttttgacttggttccaaatacaataatgtttatattttctattagtttgtaaatacggactctggatggtgcatttgtgtcttcattccataagatattcccctcccctcagccccaccccctctctatttttttctttcttttttgcaaaggtgactttctggcaacgtctttgtctctgtttggtggtgggctgctcgggctcctggacctggacttgcccccaaattttgtgtatgcagtgaaggcttcaacatctcatgaaggacactttatttctacagcagaggacacgaaaaacagataaaacaagccagtctcccattttgtacctaatcaaacaacacacatgctaagcatataaagacaagagggtggaaaatatctgaacaagaaggctctaaaggaagtcacttagaaacttaagtttaatgtgaaatgttttgcaaagatgcttaaaatgaactttgtgttaagaaaaccactgtgaaactaaattgtcctattattgttggcttacctgtgtgttcagcaatctcagccccaaataatgttgtaatttaaagaaaatggaaaattctgctctaatgaatgtaacaatggcttgctgtgaagtttacattgttgtacagaagcatgtttcgcatgtaggtaaactggtggtggtactagaaatacaatgttatttaattttaacaaattccctttattcatttctgaaattacaggacacagtttaactcataaacctttctagaccaatttatttttcactttaatgttaataacagttgtggagtatatgtgtgtgtgagcatgtgagtatgtgttgtattttaaaacaattgattttctggggcaaaattctacagtttttaatcccttctgtttaggaagttcttcctgtttggcaatataggcttaaaaatatgtttttaggacattggtacaattcagctgttggaaaattaatatattgagggttttttggtactaattctgtgcaataactaaaagagcacctcactggatatggatgttgaagatggattccctaggtgattttaatttcttccggtctgtgctgtgcacagtctacatggcaatgcggttccaccacatcggtttcgtggcttcgtttaaaactcagatggctagattagttaggttttcaaatcactaggatgtaaacagtaagcagatttctgacacacaaattatgttagagtgactgcttttttcagacagcagatatcttatagagagctttgaactgcatttatttctaaagcaaccgaaattcagtgctacaaatagaggattataacttcaggagaagaataagcagaaggagcagatgaactctcagggccatagtcttcctttgatcttgtaaaacttccattgacatctggagttcccagtctggtgagaaaatagactataaactgaatggaacaaagatccaatccaatattttggtggagacttctttaaaaccataccatacagggactctcctgtcatctgaaaaactgatgtaaggtacagaactattctttatcaaatgtttttaggtggctgttagggggctttaaaaaatattacttgcttgtgtggaaatgcaaataatgttattttctttatctaaattaagaaatctcttgttattgtgctatttataatttttttctggttcttgtattttaaaaaatctaatattaatggtattgaagtttccttttctccctctaggtcttaacagtgaattcacatggagtaatttttaaaagatatcagatacaatttgctattcaaagaaaattatgatttaaagccactttttaaaatacgagaaggaaaataggatggattaaagggttaacttttaaagattattattggttaatgttgacatatttcctctatctcatagatggtaaaagtgttgcttttaaactggcaaatgcactcttcagaaatccttttctatctgatccacatggagaggttaaaggttcaatttcatgacctctatgcaggcagcgctctcattggatgtaagaatattacctgcaaggatagaatgcagttgtgcaacagagacacattcttatttcttttttttcacaattttgttttgtttttaatgacccttttattgaatattggactgaaatataaattttaaaaaacacgttggaaaggatgtacaacagaaggctatgtatgtatatacagtatgtcaaaagccttttatttttatacttcaaatgctctaaattaataaaaagtaataatta

SEQ ID NO: 205 - protein, 333 aa, Homo sapiens
MAVRGANTLTSFSIQAILNKKEERGGLAAPEGRPAPGGTAASVAAAPAVCCWRLFGERDAGALGGAEDSLLASPAGTRTAAGRTAESPEGWDSDSALSEENESRRRCADARGASGAGLAGGSLSLGQPVCELAASKDLEEEAAGRSDSEMSASVSGDRSPRTEDDGVGPRGAHVSALCSGAGGGGGSGPAGVAEEEEEPAAPKPRKKRSRAAFSHAQVFELERRFNHQRYLSGPERADLAASLKLTETQVKIWFQNRRYKTKRRQMAADLLASAPAAKKVAVKVLVRDDQRQYLPGEVLRPPSLLPLQPSYYYPYYCLPGWALSTCAAAAGTQ

SEQ ID NO: 206 - DNA, 2259 bp, Homo sapiens
  source 1..2259: mol_type = genomic DNA
actcgcgctgcggccgccggcgctctctgtcccgctcggagctgctcggcgccccagctgcccgccccgccggccgctcctgcccgcggcgcagatggctgtgcgcggcgccaacaccttgacgtccttctccatccaggcgatcctcaacaagaaagaggagcgcggcgggctggccgcgccagaggggcgcccggcgcccgggggcacagcggcatcggtggccgcggctcccgctgtctgctgttggcggctctttggggagagggacgcgggcgcgttggggggcgccgaggactctctgctggcgtctcctgccggtaccagaacagctgcggggcggactgcggagagcccggaaggctgggactcggactccgcgctcagcgaggagaacgagagcaggcggcgctgcgcggacgcgcggggggccagcggggccggccttgcggggggatccttgagcctcggccagccggtctgtgagctggccgcttccaaagacctagaggaggaagccgcgggccggagcgacagcgagatgtccgccagcgtctcaggcgaccgcagcccaaggaccgaggacgacggtgttggccccagaggtgcacacgtgtccgcgctgtgcagcggggccggcggcgggggcggcagcgggccggcaggcgtcgcggaggaggaggaggagccggcggcgcccaagccacgcaagaagcgctcgcgggccgctttctcccacgcgcaggtcttcgagctggagcgccgctttaaccaccagcgctacctgtccgggcccgagcgcgcagacctggccgcgtcgctgaagctcaccgagacgcaggtgaaaatctggttccagaaccgtcgctacaagacaaagcgccggcagatggcagccgacctgctggcctcggcgcccgccgccaagaaggtggccgtaaaggtgctggtgcgcgacgaccagagacaatacctgcccggcgaagtgctgcggccaccctcgcttctgccactgcagccctcctactattacccgtactactgcctcccaggctgggcgctctccacctgcgcagctgccgcaggcacccagtgaacccgcttgggctgaggcagcgagtgattcccgcgctccggctccggaccggcgctgacagctgtaggctgtagcctgcacggggcgccccgccaaggaggcacctggaggtgaaacccagctccagctcccgttagccaggacttgtcccctggcagctgggctgagtctgccctgagggggcgcctttttctaatttgaacagaggcaccctatggcctaggggccctgatcgcccacctgcctggaagcccctgggctctatttattatcatgacaatgttggaattaaattttgattcgaatatgtctgcctgggggtggggttttccctgagcggcaactcctggagaccacatagcctgaatcctcagaatttcaggcctgctgggagctttctgcactaggccacactagttcatggtatccatgctaccaatctatgtgtatctacatatcttttatttttggaaattgcatttgtaaccaaggggtgcgaaaccctggcagtcccaggcagcaccaggccaggggttgatttgaaacgtgaaggattgggttttcaggccctctgctccacccctcctgtgtgtcagagctagggtgggggtgcccgattcgggtgctgaatgtaaggaggggagcctccaagtgtggtgcaagccgggggtctccacatcttccttctctgaagtccaggtacctgcacaagcaggaagcgcctgggagtcccggaaggaggagagcgcacacccaggcagccctctgcggaaactttccttggtttctttttatttgtgtaaaggaggttaagacgtgtcgcacttttcagttgtttgtattcaaatgacgattatttttctactcaatgtgaatatccctggccagcctttccacggcgcccaccgcagtgccgctgcctggccctcagtgtctaccttctgccctctgcgactccagtgctctggcccgggactcccctatccgcccctcacttacccttaaacaggtgatcccacctgtcttgtcaacctcgccgcttttcgcctccttaatggcactgtgcactcaactagagtattaactgtaaaaagatttgtgaagtttggaagctctattcgctgtattttttctttaatttataaacttttagtttaacatgc

SEQ ID NO: 207 - protein, 350 aa, Homo sapiens
MMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADS

SEQ ID NO: 208 - DNA, 2104 bp, Homo sapiens
  source 1..2104: mol_type = genomic DNA
aaccgcatctgcagcgagcatctgagaagccaagactgagccggcggccgcggcgcagcgaacgagcagtgaccgtgctcctacccagctctgctccacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggacttctgcacggacctggccgtctccagtgccaacttcattcccacggtcactgccatctcgaccagtccggacctgcagtggctggtgcagcccgccctcgtctcctccgtggccccatcgcagaccagagcccctcaccctttcggagtccccgccccctccgctggggcttactccagggctggcgttgtgaagaccatgacaggaggccgagcgcagagcattggcaggaggggcaaggtggaacagttatctccagaagaagaagagaaaaggagaatccgaagggaaaggaataagatggctgcagccaaatgccgcaaccggaggagggagctgactgatacactccaagcggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaaagcatttaagttgaatgcga

SEQ ID NO: 209 - protein, 302 aa, Homo sapiens
MFQAFPGDYDSGSRCSSSPSAESQYLSSVDSFGSPPTAAASQECAGLGEMPGSFVPTVTAITTSQDLQWLVQPTLISSMAQSQGQPLASQPPVVDPYDMPGTSYSTPGMSGYSSGGASGSGGPSTSGTTSGPGPARPARARPRRPREETETDQLEEEKAELESEIAELQKEKERLEFVLVAHKPGCKIPYEEGPGPGPLAEVRDLPGSAPAKEDGFSWLLPPPPPPPLPFQTSQDAPPNLTASLFTHSEVQVLGDPFPVVNPSYTSSFVLTCPEVSAFAGAQRTSGSDQPSDPLNSPSLLAL

SEQ ID NO: 210 - DNA, 3667 bp, Homo sapiens
  source 1..3667: mol_type = genomic DNA
attcataagactcagagctacggccacggcagggacacgcggaaccaagacttggaaacttgattgttgtggttcttcttgggggttatgaaatttcattaatctttttttttccggggagaaagtttttggaaagattcttccagatatttcttcattttcttttggaggaccgacttactttttttggtcttctttattactcccctccccccgtgggacccgccggacgcgtggaggagaccgtagctgaagctgattctgtacagcgggacagcgctttctgcccctgggggagcaacccctccctcgcccctgggtcctacggagcctgcactttcaagaggtacagcggcatcctgtgggggcctgggcaccgcaggaagactgcacagaaactttgccattgttggaacgggacgttgctccttccccgagcttccccggacagcgtactttgaggactcgctcagctcaccggggactcccacggctcaccccggacttgcaccttacttccccaacccggccatagccttggcttcccggcgacctcagcgtggtcacaggggcccccctgtgcccagggaaatgtttcaggctttccccggagactacgactccggctcccggtgcagctcctcaccctctgccgagtctcaatatctgtcttcggtggactccttcggcagtccacccaccgccgccgcctcccaggagtgcgccggtctcggggaaatgcccggttccttcgtgcccacggtcaccgcgatcacaaccagccaggacctccagtggcttgtgcaacccaccctcatctcttccatggcccagtcccaggggcagccactggcctcccagcccccggtcgtcgacccctacgacatgccgggaaccagctactccacaccaggcatgagtggctacagcagtggcggagcgagtggcagtggtgggccttccaccagcggaactaccagtgggcctgggcctgcccgcccagcccgagcccggcctaggagaccccgagaggagacggagacagatcagttggaggaagaaaaagcagagctggagtcggagatcgccgagctccaaaaggagaaggaacgtctggagtttgtgctggtggcccacaaaccgggctgcaagatcccctacgaagaggggcccgggccgggcccgctggcggaggtgagagatttgccgggctcagcaccggctaaggaagatggcttcagctggctgctgccgcccccgccaccaccgcccctgcccttccagaccagccaagacgcaccccccaacctgacggcttctctctttacacacagtgaagttcaagtcctcggcgaccccttccccgttgttaacccttcgtacacttcttcgtttgtcctcacctgcccggaggtctccgcgttcgccggcgcccaacgcaccagcggcagtgaccagccttccgatcccctgaactcgccctccctcctcgctctgtgaactctttagacacacaaaacaaacaaacacatgggggagagagacttggaagaggaggaggaggaggagaaggaggagagagaggggaagagacaaagtgggtgtgtggcctccctggctcctccgtctgaccctctgcggccactgcgccactgccatcggacaggaggattccttgtgttttgtcctgcctcttgtttctgtgccccggcgaggccggagagctggtgactttggggacagggggtgggaaggggatggacacccccagctgactgttggctctctgacgtcaacccaagctctggggatgggtggggaggggggcgggtgacgcccaccttcgggcagtcctgtgtgaggattaagggacgggggtgggaggtaggctgtggggtgggctggagtcctctccagagaggctcaacaaggaaaaatgccactccctacccaatgtctcccacacccaccctttttttggggtgcctaggttggtttcccctgcactcccgaccttagcttattgatcccacatttccatggtgtgagatcctctttactctgggcagaagtgagccccccccttaaagggaattcgatgcccccctagaataatctcatccccccacccgacttcttttgaaatgtgaacgtccttccttgactgtctagccactccctcccagaaaaactggctctgattggaatttctggcctcctaaggctccccaccccgaaatcagcccccagccttgtttctgatgacagtgttatcccaagaccctgccccctgccagccgaccctcctggccttcctcgttgggccgctctgatttcaggcagcaggggctgctgtgatgccgtcctgctggagtgatttatactgtgaaatgagttggccagattgtggggtgcagctgggtggggcagcacacctctggggggataatgtccccactcccgaaagcctttcctcggtctcccttccgtccatcccccttcttcctcccctcaacagtgagttagactcaagggggtgacagaaccgagaagggggtgacagtcctccatccacgtggcctctctctctctcctcaggaccctcagccctggcctttttctttaaggtcccccgaccaatccccagcctaggacgccaacttctcccaccccttggcccctcacatcctctccaggaagggagtgaggggctgtgacatttttccggagaagatttcagagctgaggctttggtacccccaaacccccaatatttttggactggcagactcaaggggctggaatctcatgattccatgcccgagtccgcccatccctgaccatggttttggctctcccaccccgccgttccctgcgcttcatctcatgaggatttctttatgaggcaaatttatattttttaatatcggggggtggaccacgccgccctccatccgtgctgcatgaaaaacattccacgtgccccttgtcgcgcgtctcccatcctgatcccagacccattccttagctatttatccctttcctggtttccgaaaggcaattatatctattatgtataagtaaatatattatatatggatgtgtgtgtgtgcgtgcgcgtgagtgtgtgagcgcttctgcagcctcggcctaggtcacgttggccctcaaagcgagccgttgaattggaaactgcttctagaaactctggctcagcctgtctcgggctgacccttttctgatcgtctcggcccctctgattgttcccgatggtctctctccctctgtcttttctcctccgcctgtgtccatctgaccgttttcacttgtctcctttctgactgtccctgccaatgctccagctgtcgtctgactctgggttcgttggggacatgagattttattttttgtgagtgagactgagggatcgtagatttttacaatctgtatctttgacaattctgggtgcgagtgtgagagtgtgagcagggcttgctcctgccaaccacaattcaatgaatccccgacccccctaccccatgctgtacttgtggttctctttttgtattttgcatctgaccccggggggctgggacagattggcaatgggccgtcccctctccccttggttctgcactgttgccaataaaaagctcttaaaaacgca

SEQ ID NO: 211 - protein, 235 aa, Homo sapiens
MFRDFGEPGPSSGNGGGYGGPAQPPAAAQAAQQKFHLVPSINTMSGSQELQWMVQPHFLGPSSYPRPLTYPQYSPPQPRPGVIRALGPPPGVRRRPCEQETDKLEDEKSGLQREIEELQKQKERLELVLEAHRPICKIPEGAKEGDTGSTSGTSSPPAPCRPVPCISLSPGPVLEPEALHTPTLMTTPSLTPFTPSLVFTYPSTPEPCASAHRKSSSSSGDPSSDPLGSPTLLAL

SEQ ID NO: 212 - DNA, 1594 bp, Homo sapiens
  source 1..1594: mol_type = genomic DNA
gagtcagaacccagcagccgtgtaccccgcagagccgccagccccgggcatgttccgagacttcggggaacccggcccgagctccgggaacggcggcgggtacggcggccccgcgcagcccccggccgcagcgcaggcagcccagcagaagttccacctggtgccaagcatcaacaccatgagtggcagtcaggagctgcagtggatggtacagcctcatttcctggggcccagcagttaccccaggcctctgacctaccctcagtacagccccccacaaccccggccaggagtcatccgggccctggggccgcctccaggggtacgtcgaaggccttgtgaacaggagactgacaaactggaagatgagaaatctgggctgcagcgagagattgaggagctgcagaagcagaaggagcgcctagagctggtgctggaagcccaccgacccatctgcaaaatcccggaaggagccaaggagggggacacaggcagtaccagtggcaccagcagcccaccagccccctgccgccctgtaccttgtatctccctttccccagggcctgtgcttgaacctgaggcactgcacacccccacactcatgaccacaccctccctaactcctttcacccccagcctggtcttcacctaccccagcactcctgagccttgtgcctcagctcatcgcaagagtagcagcagcagcggagacccatcctctgacccccttggctctccaaccctcctcgctttgtgaggcgcctgagccctactccctgcagatgccaccctagccaatgtctcctccccttcccccaccggtccagctggcctggacagtatcccacatccaactccagcaacttcttctccatccctctaatgagactgaccatattgtgcttcacagtagagccagcttggggccaccaaagctgcccactgtttctcttgagctggcctctctagcacaatttgcactaaatcagagacaaaatatttcccatttgtgccagaggaatcctggcagcccagagactttgtagatccttagaggtcctctggagccctaaccccttccagatcactgccacactctccatcaccctcttcctgtgatccacccaaccctatctcctgacagaaggtgccactttacccacctagaacactaactcaccagccccactgccagcagcagcaggtgattggaccaggccattctgccgccccctcctgaaccgcacagctcaggaggcgcccttggcttctgtgatgagctgatctgcggatctcagctttgagaagccttcagctccagggaatccaagcctccacagcgagggcagctgctatttattttcctaaagagagtatttttatacaaacctaccaaaatggaataaaaggcttgaagctgtggcctgagtgcctcactggacccagaggccaatgggagagtatttggagccctaggtcccagccttagctctacagactcactgcatgaccttggacaaattctttgatatttttggactttgtcttatctgacaagtggggctacatccgctcggcctcatctccgggactg

SEQ ID NO: 213 - protein, 326 aa, Homo sapiens
MYQDYPGNFDTSSRGSSGSPAHAESYSSGGGGQQKFRVDMPGSGSAFIPTINAITTSQDLQWMVQPTVITSMSNPYPRSHPYSPLPGLASVPGHMALPRPGVIKTIGTTVGRRRRDEQLSPEEEEKRRIRRERNKLAAAKCRNRRRELTEKLQAETEELEEEKSGLQKEIAELQKEKEKLEFMLVAHGPVCKISPEERRSPPAPGLQPMRSGGGSVGAVVVKQEPLEEDSPSSSSAGLDKAQRSVIKPISIAGGFYGEEPLHTPIVVTSTPAVTPGTSNLVFTYPSVLEQESPASPSESCSKAHRRSSSSGDQSSDSLNSPTLLAL

SEQ ID NO: 214 - DNA, 6711 bp, Homo sapiens
  source 1..6711: mol_type = genomic DNA
gtagtgactcatctcgggcagagcgctagggctccgagcgaaccagcgagcgagcgaacgagcggcgctcggcggggacagaaagagggagagagagagagagagagagggagaggcgcggccgggcgaggcgggcccgtccgggagcgggctccggggaaggggtgcgggtctgggcgccggagcggggagcggggccgcgtccctctcagcgccagctctacttgagccccacgagccgctgtccccctggcgcgctcggggccgcgggacgggcgcacgccgccttctcctagtcaagtatccgagccgccccgaaactcgggcggcgagtcggccacgggaagtttattctccggctccttttctaaaaggaagaaacagaagtttctcccagcggacagcttttctttccgcctttttggccctgtctgaaatcgggggtccccagggctggcaggccaggctcgctgggctcctaatcttttttttaatttccaatttttgattgggccgtgggtccccgctgagctccggctgcgcgcgggggcgggagggcgcgcgcaggggagggaccgagagacgcgccgactttttagagggagggatcgggtggacaactggtcccgcggcgctcgcagagccggaaagaagtgctgtaagggacgctcgggggacgctgttcctgaggtgtcgccgcctccctgtcctcgccctccgcggtgggggagaaacccaggagcgaagcccagagcccgcggcgcggccggcggacgaacgagcgcgcagcagccggtgcgcggccgcggcgagggcgggggaagaaaaacaccctgtttcctctccggcccccaccgcggatcatgtaccaggattatcccgggaactttgacacctcgtcccggggcagcagcggctctcctgcgcacgccgagtcctactccagcggcggcggcggccagcagaaattccgggtagatatgcctggctcaggcagtgcattcatccccaccatcaacgccatcacgaccagccaggacctgcagtggatggtgcagcccacagtgatcacctccatgtccaacccataccctcgctcgcacccctacagccccctgccgggcctggcctctgtccctggacacatggccctcccaagacctggcgtgatcaagaccattggcaccaccgtgggccgcaggaggagagatgagcagctgtctcctgaagaggaggagaagcgtcgcatccggcgggagaggaacaagctggctgcagccaagtgccggaaccgacgccgggagctgacagagaagctgcaggcggagacagaggagctggaggaggagaagtcaggcctgcagaaggagattgctgagctgcagaaggagaaggagaagctggagttcatgttggtggctcacggcccagtgtgcaagattagccccgaggagcgccgatcgcccccagcccctgggctgcagcccatgcgcagtgggggtggctcggtgggcgctgtagtggtgaaacaggagcccctggaagaggacagcccctcgtcctcgtcggcggggctggacaaggcccagcgctctgtcatcaagcccatcagcattgctgggggcttctacggtgaggagcccctgcacacccccatcgtggtgacctccacacctgctgtcactccgggcacctcgaacctcgtcttcacctatcctagcgtcctggagcaggagtcacccgcatctccctccgaatcctgctccaaggctcaccgcagaagcagtagcagcggggaccaatcatcagactccttgaactcccccactctgctggctctgtaacccagtgcacctccctccccagctccggagggggtcctcctcgctcctccttcccagggaccagcaccttcaagcgctccagggccgtgagggcaagagggggacctgccaccagggagcttcctggctctgggggacccaggtgggacttagcagtgagtattggaagacttgggttgatctcttagaagccatgggacctcctccctcattcatcttgcaagcaaatcccatttcttgaaaagccttggagaactcggtttggtagacttggacatctctctggcttctgaagagcctgaagctggcctggaccattcctgtccctttgttaccatactgtctctggagtgatggtgtccttccctgccccaccacgcatgctcagtgccttttggtttcaccttccctcgacttgaccctttcctcccccagcgtcagtttcactccctcttggtttttatcaaatttgccatgacatttcatctgggtggtctgaatattaaagctcttcatttctggagatggggcagcaggtggctcttctgctggggctgacttgtccagaaggggacaaagtgcaatacagagccttccctaccctgacgcctcccagtcatcatctccagaactcccagcggggctccctgagctctcaaggagatgctgccatcactgggaggctcagaggacccttcctgcccaccttcggagacggcttctggaggaacggcttggccagaagacagggtgtgagtgagacagtggggcacaggttgggtttgccaaacgcctaattaccaggccaggaagcatgccaacaaagccacacgggtgtcctagccagcttcccttcacctggtgtcttgagtagggcgtctcctgtaattactgccttgccattctgcccctggacccttctctccggaccagggaggcgtccctccctaggagccacacattatactccaagtccctgccgggctccgcctttcccccaccctggctctcagggtgacgccacccacagagatttaatgagcgtgggcctggaccttccccagatgctgccaggcagcccctccccaagcctcaaagaagcatttgctgaggatggagaggcaggggagggaggcgggaggccgtcactggagtggcgtctgcagcagctgctgccccagcacccgctcagcctgtcctggctgctcacctccccgcagggcaccgggcctttcctgccctctgtggtcatctgccacctgctggatcaagtgctttctcttttacactcccctgtccccaccccagtgcactcttctggcccaggcagcaagcaagctgtgaacagctggcctgagctgtcgctgtggcttgtggctcatgcgccattcctggttgtctgttgaatctttctggctgctggaattggagataggatgttttgcttcccactgcaggagagctgccccctttcacggggttggggaagggtccccctggcctccagcaggagcacagctcagcagggtccctgctgcccacccctctgagccttttctccccagggtatggctcctgctgagtttcttgtccagcagggccttgacaggaatccagggagtagctcctggccagaaccagcctctgcggggcttgtgctctgcaaagactctgctgctggggattcagctctagaggtcacagtatcctcgtttgaaagataattagatcccccgtggagaaagcagtgacacattcacacagctgttccctcgcatgttatttcatgaacatgcctgttttcgtgcactagacacacagagtggaacagccgtatgcttaaagtacatgggccagtgggactggaagtgacctgtacaagtgatgcagaaaggagggtttcaaagaaaaaggattttgtttaaaatactttaaaaatgttatttcctgcatcccttggctgtgatgcccctctcccgatttcccaggggctctgggagggacccttctaagaagattgggcagttgggtttctggcttgagatgaatccaagcagcagaatgagccaggagtagcaggagatgggcaaagaaaactggggtgcactcagctctcacaggggtaatcatctcaagtggtatttgtagccaagtgggagctattttcttttttgtgcatatagatatttcttaaatgaagctgctttcttgtcttttatttctaaaagcccccttataccccactttgtgcagcaaagatccccgtgcaggtcacagcctgatttgtggccaggctggacaaattcctgaggcacaacttggcttcagttcagatttcaagctgtgttggtgttgggaccagcagaaggcaaacgtccagccaacacacaggactgtaagaggactctgagctacgtgccctgtgaagacccccaggctttgtcataggaggtcgttcagcttccccaaagtcagaggtgatttgatttggggaagactgaatattcacacctaagtcgtgagcatatcctgagttttacttccttatggcttgccctccaagttctctctctcatacacacacacacccttgctccagaatcaccagacacctccatggctccagctatgggaacagctgcattggggctgcctttctgtttggcttaggaacttctgtgcttcttgtggctccactcgcgaggcagctcggaggtgtggactccgattgggctgcaggcagctctgggacggcacagggcgggcgctctgatcagctcgtgtaaaacacaccgtcttcttggcctcctggccagtctttctgcgaatagtcctctccctggccagttgaatgggggaagctgctggcacaggaaggagaggcgatcccggctgaggcttaggaaattgctggagccggctccaagcagataattcactggggaggttttcagagtcaaacatcattctgcctgtgttgggggccaggtgtgtcacacaagcatctcaaagtcaaaagccatctggggctgctgcttctgtttctcaggctctggggaaaggaatctccctctcctctcacttgattccaagtgtggttgaattgtctggagcactgggactttttttctcttttccttgatggaccaacagtgcaaatgcaatctcgccatttaactttcaggtcgatttcctttcctgatcagacatctttgtgccccctttaggaaggaaaagaatacacctacgatgtgccaggcactgtgttaggcgcttttatatagatcctcgttaggatgagactaagggatgaggacatctctttataaaaggcccctaagtaatggataaacagaaacacttagaggtgagaaggtctgtcttcaagatccaaggtaagattgccttcagtctgatgtttgttctcaaggacttatcccctacaatattctcccactccatacttctccttctaccccaccatgtgctcccgtgcactcctcagatggtcagaggggtaacccaagtccttagagaatttggggaccaatagaatatgtgatgtgtgaattttctttaaaaaacttaaggagtctttgctaccttctgcttgttgagttgttttggcattcatattaaaagccagcatctcactatttattgacaggttgggctgtgtgtgtgcgcatgtgtgtatacatttccaggcgtgcctgtgtcctgtagctttttaaaaggaaacccagtcatcccactatgaatctggcatcttcttatgcttctagtgttttggccatacatcaaccaaggggtttaatttatccaatgcttgacgacatgttcaggaggggctggatcaaattttgagagggttatgggaaagggagggggagaagaaattgacatttattttattatttattttaaatgtttacatcttctttatgttgtatcaagcctgaatagaaactgatagcattaaaatactccgttcctctctctcttctcgcttcctttttttttttttttttaaatttaggataacacatttttgtttctaaagtgatttgtgatttgtgctgtataaactgtataaaaggttctgtttttaaaggtggattttcattcctctggggacagtggtcgccaagacatctacattgtaagagaacacagtggaagatcctgtcctgattctcaaaaattattttctctgtatgattaaaagtttattccatttattttagtttgtgtttacttgattttgaggaagaaaatatttgactttgtgtaaagagtagggtatcagggtgtcttttctgccgtgggagatgtgtatatatatagtattttggtgtatagtagaaaataagctttgtgcatctgtatttgagatatgttaatgacgtggagtaaagtcagctgtaagactctggaggcaaacaagttgtatatggttcatatggctctatggggaatttaattacctttctgggcactttttttttttttttttttttaagtaatggtgaaatggtcccattggagagtctcctaaatagaccttccaggcagaaccgcaagctcaaaatctttgtatagttttgaaaattgaggagtagctttgtttggaagcctttctggtggtggtttttgttgttgttgttgttttgttgttttactatatgtaatacaagcctacagtatttgcactaaagaaagcttgttagaaaaagcttgctgctatggaagaaagaacatattaaaacttctttcccttgcgatttttttgggggaggggggttagcatttccactttcagttgagtagcattttgtagaataaaatgaattaagattgaagagcc

SEQ ID NO: 215 - protein, 331 aa, Homo sapiens
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 216 - DNA, 3257 bp, Homo sapiens
  source 1..3257: mol_type = genomic DNA
gctcagagttgcactgagtgtggctgaagcagcgaggcgggagtggaggtgcgcggagtcaggcagacagacagacacagccagccagccaggtcggcagtatagtccgaactgcaaatcttattttcttttcaccttctctctaactgcccagagctagcgcctgtggctcccgggctggtgtttcgggagtgtccagagagcctggtctccagccgcccccgggaggagagccctgctgcccaggcgctgttgacagcggcggaaagcagcggtacccacgcgcccgccgggggaagtcggcgagcggctgcagcagcaaagaactttcccggctgggaggaccggagacaagtggcagagtcccggagccaacttttgcaagcctttcctgcgtcttaggcttctccacggcggtaaagaccagaaggcggcggagagccacgcaagagaagaaggacgtgcgctcagcttcgctcgcaccggttgttgaacttgggcgagcgcgagccgcggctgccgggcgccccctccccctagcagcggaggaggggacaagtcgtcggagtccgggcggccaagacccgccgccggccggccactgcagggtccgcactgatccgctccgcggggagagccgctgctctgggaagtgagttcgcctgcggactccgaggaaccgctgcgcacgaagagcgctcagtgagtgaccgcgacttttcaaagccgggtagcgcgcgcgagtcgacaagtaagagtgcgggaggcatcttaattaaccctgcgctccctggagcgagctggtgaggagggcgcagcggggacgacagccagcgggtgcgtgcgctcttagagaaactttccctgtcaaaggctccggggggcgcgggtgtcccccgcttgccacagccctgttgcggccccgaaacttgtgcgcgcagcccaaactaacctcacgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgaagagagaccgtcgggggctgaggggcaacgaagaaaaaaaataacacagagagacagacttgagaacttgacaagttgcgacggagagaaaaaagaagtgtccgagaactaaagccaagggtatccaagttggactgggttgcgtcctgacggcgcccccagtgtgcacgagtgggaaggacttggcgcgccctcccttggcgtggagccagggagcggccgcctgcgggctgccccgctttgcggacgggctgtccccgcgcgaacggaacgttggacttttcgttaacattgaccaagaactgcatggacctaacattcgatctcattcagtattaaaggggggagggggagggggttacaaactgcaatagagactgtagattgcttctgtagtactccttaagaacacaaagcggggggagggttggggaggggcggcaggagggaggtttgtgagagcgaggctgagcctacagatgaactctttctggcctgccttcgttaactgtgtatgtacatatatatattttttaatttgatgaaagctgattactgtcaataaacagcttcatgcctttgtaagttatttcttgtttgtttgtttgggtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatactagctatctaggtggagttgaaagagttaagaatgtcgattaaaatcactctcagtgcttcttactattaagcagtaaaaactgttctctattagactttagaaataaatgtacctgatgtacctgatgctatggtcaggttatactcctcctcccccagctatctatatggaattgcttaccaaaggatagtgcgatgtttcaggaggctggaggaaggggggttgcagtggagagggacagcccactgagaagtcaaacatttcaaagtttggattgtatcaagtggcatgtgctgtgaccatttataatgttagtagaaattttacaataggtgcttattctcaaagcaggaattggtggcagattttacaaaagatgtatccttccaatttggaatcttctctttgacaattcctagataaaaagatggcctttgcttatgaatatttataacagcattcttgtcacaataaatgtattcaaataccaa

SEQ ID NO: 217 - protein, 347 aa, Homo sapiens
MCTKMEQPFYHDDSYTATGYGRAPGGLSLHDYKLLKPSLAVNLADPYRSLKAPGARGPGPEGGGGGSYFSGQGSDTGASLKLASSELERLIVPNSNGVITTTPTPPGQYFYPRGGGSGGGAGGAGGGVTEEQEGFADGFVKALDDLHKMNHVTPPNVSLGATGGPPAGPGGVYAGPEPPPVYTNLSSYSPASASSGGAGAAVGTGSSYPTTTISYLPHAPPFAGGHPAQLGLGRGASTFKEEPQTVPEARSRDATPPVSPINMEDQERIKVERKRLRNRLAATKCRKRKLERIARLEDKVKTLKAENAGLSSTAGLLREQVAQLKQKVMTHVSNGCQLLLGVKGHAF

SEQ ID NO: 218 - DNA, 1830 bp, Homo sapiens
  source 1..1830: mol_type = genomic DNA
gggaccttgagagcggccaggccagcctcggagccagcagggagctgggagctgggggaaacgacgccaggaaagctatcgcgccagagagggcgacgggggctcgggaagcctgacagggcttttgcgcacagctgccggctggctgctacccgcccgcgccagcccccgagaacgcgcgaccaggcacccagtccggtcaccgcagcggagagctcgccgctcgctgcagcgaggcccggagcggccccgcagggaccctccccagaccgcctgggccgcccggatgtgcactaaaatggaacagcccttctaccacgacgactcatacacagctacgggatacggccgggcccctggtggcctctctctacacgactacaaactcctgaaaccgagcctggcggtcaacctggccgacccctaccggagtctcaaagcgcctggggctcgcggacccggcccagagggcggcggtggcggcagctacttttctggtcagggctcggacaccggcgcgtctctcaagctcgcctcttcggagctggaacgcctgattgtccccaacagcaacggcgtgatcacgacgacgcctacacccccgggacagtacttttacccccgcgggggtggcagcggtggaggtgcagggggcgcagggggcggcgtcaccgaggagcaggagggcttcgccgacggctttgtcaaagccctggacgatctgcacaagatgaaccacgtgacaccccccaacgtgtccctgggcgctaccggggggcccccggctgggcccgggggcgtctacgccggcccggagccacctcccgtttacaccaacctcagcagctactccccagcctctgcgtcctcgggaggcgccggggctgccgtcgggaccgggagctcgtacccgacgaccaccatcagctacctcccacacgcgccgcccttcgccggtggccacccggcgcagctgggcttgggccgcggcgcctccaccttcaaggaggaaccgcagaccgtgccggaggcgcgcagccgggacgccacgccgccggtgtcccccatcaacatggaagaccaagagcgcatcaaagtggagcgcaagcggctgcggaaccggctggcggccaccaagtgccggaagcggaagctggagcgcatcgcgcgcctggaggacaaggtgaagacgctcaaggccgagaacgcggggctgtcgagtaccgccggcctcctccgggagcaggtggcccagctcaaacagaaggtcatgacccacgtcagcaacggctgtcagctgctgcttggggtcaagggacacgccttctgaacgtcccctgcccctttacggacaccccctcgcttggacggctgggcacacgcctcccactggggtccagggagcaggcggtgggcacccaccctgggacctaggggcgccgcaaaccacactggactccggccctcctaccctgcgcccagtccttccacctcgacgtttacaagcccccccttccacttttttttgtatgttttttttctgctggaaacagactcgattcatattgaatataatatatttgtgtatttaacagggaggggaagagggggcgatcgcggcggagctggccccgccgcctggtactcaagcccgcggggacattgggaaggggacccccgccccctgccctcccctctctgcaccgtactgtggaaaagaaacacgcacttagtctctaaagagtttattttaagacgtgtttgtgtttgtgtgtgtttgttctttttattgaatctatttaagtaaaaaaaaaattggttctttattaa

SEQ ID NO: 219 - protein, 304 aa, Homo sapiens
MMKKDALTLSLSEQVAAALKPAAAPPPTPLRADGAPSAAPPDGLLASPDLGLLKLASPELERLIIQSNGLVTTTPTSSQFLYPKVAASEEQEFAEGFVKALEDLHKQNQLGAGAAAAAAAAAAGGPSGTATGSAPPGELAPAAAAPEAPVYANLSSYAGGAGGAGGAATVAFAAEPVPFPPPPPPGALGPPRLAALKDEPQTVPDVPSFGESPPLSPIDMDTQERIKAERKRLRNRIAASKCRKRKLERISRLEEKVKTLKSQNTELASTASLLREQVAQLKQKVLSHVNSGCQLLPQHQVPAY

SEQ ID NO: 220 - DNA, 1929 bp, Homo sapiens
  source 1..1929: mol_type = genomic DNA
aggagccgccgccagtggagggccgggcgctgcggccgcggccggggcgggcgcagggccgagcggacgggggggcgcgggccccccgggaggccgcggccactcccccccgggccggcgcggcgggggaggcggaggatggaaacacccttctacggcgatgaggcgctgagcggcctgggcggcggcgccagtggcagcggcggcagcttcgcgtccccgggccgcttgttccccggggcgcccccgacggccgcggccggcagcatgatgaagaaggacgcgctgacgctgagcctgagtgagcaggtggcggcagcgctcaagcctgcggccgcgccgcctcctacccccctgcgcgccgacggcgcccccagcgcggcaccccccgacggcctgctcgcctctcccgacctggggctgctgaagctggcctcccccgagctcgagcgcctcatcatccagtccaacgggctggtcaccaccacgccgacgagctcacagttcctctaccccaaggtggcggccagcgaggagcaggagttcgccgagggcttcgtcaaggccctggaggatttacacaagcagaaccagctcggcgcgggcgcggccgctgccgccgccgccgccgccgccggggggccctcgggcacggccacgggctccgcgccccccggcgagctggccccggcggcggccgcgcccgaagcgcctgtctacgcgaacctgagcagctacgcgggcggcgccgggggcgcggggggcgccgcgacggtcgccttcgctgccgaacctgtgcccttcccgccgccgccacccccaggcgcgttggggccgccgcgcctggctgcgctcaaggacgagccacagacggtgcccgacgtgccgagcttcggcgagagcccgccgttgtcgcccatcgacatggacacgcaggagcgcatcaaggcggagcgcaagcggctgcgcaaccgcatcgccgcctccaagtgccgcaagcgcaagctggagcgcatctcgcgcctggaagagaaagtgaagaccctcaagagtcagaacacggagctggcgtccacggcgagcctgctgcgcgagcaggtggcgcagctcaagcagaaagtcctcagccacgtcaacagcggctgccagctgctgccccagcaccaggtgcccgcgtactgagtccgcgcgcggggcgcatgcgcggccaccctccccaaggggcgggctcgcgggggggtgtcgtgggcgccccggacttggagagggtgcggccctggggaccccccctccccgagtgtgcccaggaactcagagagggcgcggcccccggggattccccccccccgagggtgcccaggactcgacaagctggaccccctgctcccgggggggcgagcgcatgacccccccgccctcgcgctgcctctttcccccgcgcggccgccccgtgttgcacaaacccgcgcgtctcggctgcccctttgtacaccgcgccgcggaagggggctccgagggggcgcagcctcaaaccctgcctttcctttacttttacttttttttttttttctttggaagagagaagaacagagtgttcgattctgccctatttatgtttctactcgggaacaaacgttggttgtgtgtgtgtgtgttttcttgtgttggttttttaaagaaatgggaagaagaaaaaaaaattctccgcccctttcctcgatctcgctccccccttcggttctttcgaccggtcccccctcccttttttgttctgttttgttttgttttgctacgagtccacattcctgtttgtaatccttggttcgcccggttttctgttttcagtaaagtctcgttacgccagctcggctctccgcctccttcttcccccgccggggcctggcgggctgggcggggcctggttcgctt

SEQ ID NO: 221 - protein, 326 aa, Homo sapiens
MDLTAIYESLLSLSPDVPVPSDHGGTESSPGWGSSGPWSLSPSDSSPSGVTSRLPGRSTSLVEGRSCGWVPPPPGFAPLAPRLGPELSPSPTSPTATSTTPSRYKTELCRTFSESGRCRYGAKCQFAHGLGELRQANRHPKYKTELCHKFYLQGRCPYGSRCHFIHNPSEDLAAPGHPPVLRQSISFSGLPSGRRTSPPPPGLAGPSLSSSSFSPSSSPPPPGDLPLSPSAFSAAPGTPLARRDPTPVCCPSCRRATPISVWGPLGGLVRTPSVQSLGSDPDEYASSGSSLGGSDSPVFEAGVFAPPQPVAAPRRLPIFNRISVSE

SEQ ID NO: 222 - DNA, 1747 bp, Homo sapiens
  source 1..1747: mol_type = genomic DNA
agcctgacttcagcgctcccactctcggccgacacccctcatggccaaccgttacaccatggatctgactgccatctacgagagcctcctgtcgctgagccctgacgtgcccgtgccatccgaccatggagggactgagtccagcccaggctggggctcctcgggaccctggagcctgagcccctccgactccagcccgtctggggtcacctcccgcctgcctggccgctccaccagcctagtggagggccgcagctgtggctgggtgcccccaccccctggcttcgcaccgctggctccccgcctgggccctgagctgtcaccctcacccacttcgcccactgcaacctccaccaccccctcgcgctacaagactgagctatgtcggaccttctcagagagtgggcgctgccgctacggggccaagtgccagtttgcccatggcctgggcgagctgcgccaggccaatcgccaccccaaatacaagacggaactctgtcacaagttctacctccagggccgctgcccctacggctctcgctgccacttcatccacaaccctagcgaagacctggcggccccgggccaccctcctgtgcttcgccagagcatcagcttctccggcctgccctctggccgccggacctcaccaccaccaccaggcctggccggcccttccctgtcctccagctccttctcgccctccagctccccaccaccacctggggaccttccactgtcaccctctgccttctctgctgcccctggcacccccctggctcgaagagaccccaccccagtctgttgcccctcctgccgaagggccactcctatcagcgtctgggggcccttgggtggcctggttcggaccccctctgtacagtccctgggatccgaccctgatgaatatgccagcagcggcagcagcctggggggctctgactctcccgtcttcgaggcgggagtttttgcaccaccccagcccgtggcagccccccggcgactccccatcttcaatcgcatctctgtttctgagtgacaaagtgactgcccggtcagatcagctggatctcagcggggagccacgtctcttgcactgtggtctctgcatggaccccagggctgtggggacttgggggacagtaatcaagtaatccccttttccagaatgcattaacccactcccctgacctcacgctggggcaggtccccaagtgtgcaagctcagtattcatgatggtgggggatggagtgtcttccgaggttcttgggggaaaaaaaattgtagcatatttaagggaggcaatgaaccctctcccccacctcttccctgcccaaatctgtctcctagaatcttatgtgctgtgaataataggccttcactgcccctccagtttttatagacctgaggttccagtgtctcctggtaactggaacctctcctgagggggaatcctggtgctcaaattaccctccaaaagcaagtagccaaagccgttgccaaaccccacccataaatcaatgggccctttatttatgacgactttatttattctaatatgattttatagtatttatatatattgggtcgtctgcttcccttgtatttttcttcctttttttgtaatattgaaaacgacgatataattattataagtagactataatatatttagtaatatatattattaccttaaaagtctatttttgtgttttgggcatttttaaataaacaatctgagtgtaa

SEQ ID NO: 223 - protein, 592 aa, Homo sapiens
MFGIQESIQRSGSSMKEEPLGSGMNAVRTWMQGAGVLDANTAAQSGVGLARAHFEKQPPSNLRKSNFFHFVLALYDRQGQPVEIERTAFVGFVEKEKEANSEKTNNGIHYRLQLLYSNGIRTEQDFYVRLIDSMTKQAIVYEGQDKNPEMCRVLLTHEIMCSRCCDKKSCGNRNETPSDPVIIDRFFLKFFLKCNQNCLKNAGNPRDMRRFQVVVSTTVNVDGHVLAVSDNMFVHNNSKHGRRARRLDPSEGTPSYLEHAATPCIKAISPSEGWTTGGATVIIIGDNFFDGLQVIFGTMLVWSELITPHAIRVQTPPRHIPGVVEVTLSYKSKQFCKGTPGRFIYTALNEPTIDYGFQRLQKVIPRHPGDPERLPKEVILKRAADLVEALYGMPHNNQEIILKRAADIAEALYSVPRNHNQLPALANTSVHAGMMGVNSFSGQLAVNVSEASQATNQGFTRNSSSVSPHGYVPSTTPQQTNYNSVTTSMNGYGSAAMSNLGGSPTFLNGSAANSPYAIVPSSPTMASSTSLPSNCSSSSGIFSFSPANMVSAVKQKSAFAPVVRPQTSPPPTCTSTNGNSLQAISGMIVPPM

SEQ ID NO: 224 - DNA, 5408 bp, Homo sapiens
  source 1..5408: mol_type = genomic DNA
cctgcttcttcaagtgaagggtacctctacaaaaggaaactccagcccctcctgtcctccaccggcctgtgatcattacaaaaaaaaaaaaaaaaaagcaaaaaaaaaaaaaaagcacccaaaccaaaaatcaaccaaccaaacaacccccaacagccaagcatacatctctaattttattattttggtcttttcgttggattttccctttcttctttttttcgggttatcgctcagttttgagcagaggtttacattttttaaaaatttgctttccagcccgccttgatcttctaagtgcgagttcatcgtctgagaaaaaaaaaaatctctggttggcgtttttgtttcttttcttttctttcttttctttcctttttttttttttttaatttttttcaagggggaggagattttccacaagaaaaggttgttttcatgtttgggattcaggaaagcatccaacggagtggaagcagcatgaaggaagagccgctgggcagcggcatgaacgcggtgcggacgtggatgcagggcgccggggtgctggacgccaacacggcggcgcagagcggggtgggtctggcccgggctcactttgagaagcagccgccttccaatctgcggaaatccaacttcttccacttcgtcctggccctctacgacagacagggccagcccgtggagatcgagaggacagcgtttgtggggttcgtggagaaggaaaaagaagccaacagcgaaaagaccaataacggaattcactaccggcttcagcttctctacagcaatgggataaggacggagcaggatttctacgtgcgcctcattgactccatgacaaaacaagccatagtgtatgaaggccaagacaagaacccagaaatgtgccgagtcttgctcacacatgagatcatgtgcagccgctgttgtgacaagaaaagctgtggcaaccgaaatgagactccctcagatccagtgataattgacaggttcttcttgaaatttttcctcaaatgtaaccaaaattgcctaaagaatgcgggaaacccacgtgacatgcggagattccaggtcgtggtgtctacgacagtcaatgtggatggccatgtcctggcagtctctgataacatgtttgtccataataattccaagcatgggcggagggctcggaggcttgacccctcggaaggtacgccctcttatctggaacatgcagctactccctgtatcaaagccatcagcccgagtgaaggatggacgacgggaggtgcgactgtgatcatcataggggacaatttctttgatgggttacaggtcatattcggtaccatgctggtctggagtgagttgatcactcctcatgccatccgtgtgcagacccctcctcggcacatccctggtgttgtggaagtcacactgtcctacaaatctaagcagttctgcaaaggaacaccaggcagattcatttatacagcgctcaacgaacccaccatcgattatggtttccagaggttacagaaggtcattcctcggcaccctggtgaccctgagcgtttgccaaaggaagtaatactcaaaagggctgcggatctggtagaagcactgtatgggatgccacacaacaaccaggaaatcattctgaagagagcggccgacattgccgaggccctgtacagtgttccccgcaaccacaaccaactcccggcccttgctaacacctcggtccacgcagggatgatgggcgtgaattcgttcagtggacaactggccgtgaatgtctccgaggcatcacaagccaccaatcagggtttcacccgcaactcaagcagcgtatcaccacacgggtacgtgccgagcaccactccccagcagaccaactataactccgtcaccacgagcatgaacggatacggctctgccgcaatgtccaatttgggcggctcccccaccttcctcaacggctcagctgccaactccccctatgccatagtgccatccagccccaccatggcctcctccacaagcctcccctccaactgcagcagctcctcgggcatcttctccttctcaccagccaacatggtctcagccgtgaaacagaagagtgctttcgcaccagtcgtcagaccccagacctccccacctcccacctgcaccagcaccaacgggaacagcctgcaagcgatatctggcatgattgttcctcctatgtgaaagaattgccttgaagaattgtattaatgaagaggttggattctgctacagagagtaatctgatacaagtcccagagtggaacttttaactcaggcctttttaagaggaatcacaataactgcagatttttaaacaaacaaaatcaccgaccttgcaaatactgaaattggaagagggatctgcaagtgcagggtgttggttaaagttgtacctcccaagtatttgggggatatatttattctgtattgacaaaagcaaatccactttttctttttcttttttttttttaagcttaattctgcaatcatttgtcttttataaaccgtaaagctctatacaagggacactataaataagactccatgttttaatttatgatgtttttaaagctgtgtaaaggaagaatgaagtggtgatatttacaaaaaagtaaaaaaaaaaaaaaagaaaaaaaggaaaaaaaaaaaagcttgtatgggacagaataggaatgccagttagattttttagaaaaactaagggtcggcttttgcgccttaaagcatatcaaatggtagttagctcagacagtgcattttcaatatctaacttaacatgccaccccttagcagtgcaagcttatttatctcttttgtattgttgtcttaagcaactgtgtaaataaatgcagcctggaaagttaaaacgcgatgtaagtgatcacatttttccctctactcgaaaatccagtgcctctagacagattgttaaaactgcatatttaaatctgatctcattctctccttttacttaagtcagtttcttctgaagccgatggcctctcgagagcttggtggcacacatactgtgtgagaatctcctttgagacttcatggaacagggtccaggcacagaattccacactctgccctccagttaacaagccaaaacctcacgtacgctcccccatttgcaagtttaaagtttactcgtcctaaatgcagacttcatacctatcttccaaaagttgataaaagcaacgtggcaggtattttcgattttcccattccagatactgcctctatcagtcgacccttaccatttgtaacataatagattgaaacaacaggttaagtgctttggaattaagagttaaagggaaccgggggtggagaaaggaaaagaaggttgagacctccaaaacatagttttctgttctatgggaatttttgctctcattatctgggaagtgttcttaaaaataagaattaatagcaaagatgcagcaaagctctgaggatgcatttgcctgatatttttttctttgctcttgtgtttttgtatgtagttataaatactgtagattttttttttgtgattttttgccaaagttgttgttctatttatacattttaatgtcttaagacattttttcaatatcacaaaaagattttactgcgtattttgcaaagaaaaaaagctcactacctttagcttgcacatacttgcaaagttaattaaaaggctttttgttttaaaggggattttgtaaaatatccatataaataatgtatttatctttcgaatttgtacattgcttttcccttcttcctcttccttccacccccaatttattttattgtgtatgtttgctacgtgaaaagtgcgtatttgtttggtcacctacacttgtattagctgtttcaatgtgatttttaaacatttcatttatagttatttttagtattgttataaaccatgcttcagtttttttaatttccacccaaaagtcattgtctatttttgtattatttgtaagttaagaagttttttccaatatatggcaaaaaaaaaaaaatagtagcatattattcttgtagtatttagttcagtagatttaaaaaaaaatgtatcctttgctttggaagcttacaaaacaaccctaatgctgttttactctattaatatgcatggaatctctccctttggagtgacgcattttgtgcattaaattctagggagaaacttcatagaaatcaatgaacatactttctttcttaagtctgcttgtatatttcctctgtctttcacataaatataaaccagcagattggatgccttaacaatgcaaatcatattcatttcacttgtacattgtaactgtgcaccagaactgtcagtcatcactaacattctaagaaaaaagaaaaagaaaaaaagaaaaaaaaaaaaacaaagaaatcgaaaagcacaaaagaactgttttgttaccttaagacaatgtaactttttctagtagagcaagaaatcatttacaacaatgctgcaactgtgcatgcccccatatggattttgcaatggttttcactaggctgtcaagagtgcgatttttatgggttggggtgggtgggggaggggagttgtgggagggtaggggagggaggaaagctgtttttcatggtgagaaataataatgatgactaataataataaaaaaaactggaaaatgtaagcaaggtggacgcattgcttctcggtactcagaaaggttatctgaatttgcgtggtaagcgctggcctgaagatgtacacaagttaaagccatattttatctggtgagccccttaactgtttctgaaggaatgaacggtcagccgggaaggtgtccggcttagaccttgacaacagacactacccatctgtcagccatgtgcagtggttagaactcttcttgaaagtccaaagagcctttaaaatgtgtataatttgtgttttgttgcttctatttctattgattagatgaaaagacattctggccctctgatcctctttcttctccacaaaagttttacaaataaaagatgttcccctaaatacagaatatggctttaagaagaaaatgaaatgaagaattaaattaagatttcagtgttgggggaaaaaatacagcttctggatgactggattgcataacattgccctggcctcacattgtactaggatgcagcttaaatgaagtcatctctaaatctactaacctttcaccttcttatcaaaactttttgaatagacacacactgtacagttcaattgttagagaacctaactactgtagagattgttaaattttttttttttttttgcaaaaattcaagctgtaaaaacttttcaactttcacaatatttaattaaagttacttcctgtctgtga

SEQ ID NO: 225 - protein, 551 aa, Homo sapiens
MFGIQENIPRGGTTMKEEPLGSGMNPVRSWMHTAGVVDANTAAQSGVGLARAHFEKQPPSNLRKSNFFHFVLALYDRQGQPVEIERTAFVDFVEKEKEPNNEKTNNGIHYKLQLLYSNGVRTEQDLYVRLIDSMTKQAIVYEGQDKNPEMCRVLLTHEIMCSRCCDKKSCGNRNETPSDPVIIDRFFLKFFLKCNQNCLKNAGNPRDMRRFQVVVSTTVNVDGHVLAVSDNMFVHNNSKHGRRARRLDPSEATPCIKAISPSEGWTTGGATVIIIGDNFFDGLQVVFGTMLVWSELITPHAIRVQTPPRHIPGVVEVTLSYKSKQFCKGAPGRFVYTALNEPTIDYGFQRLQKVIPRHPGDPERLPKEVLLKRAADLVEALYGMPHNNQEIILKRAADIAEALYSVPRNHNQIPTLGNNPAHTGMMGVNSFSSQLAVNVSETSQANDQVGYSRNTSSVSPRGYVPSSTPQQSNYNTVSTSMNGYGSGAMASLGVPGSPGFLNGSSANSPYGMKQKSAFAPVVRPQASPPPSCTSANGNGLQAMSGLVVPPM

SEQ ID NO: 226 - DNA, 4858 bp, Homo sapiens
  source 1..4858: mol_type = genomic DNA
attcctcggggagagagggctgagttttgtgcgcctccgtccgctgcgcgcgccgctccaggccccgccgcgccccgcctgcgcccgggactgcgccgcagcactcactgccccgtttatttgtctttggagcaggcgggccgcgccagaagcgggcgatcccgcagtgtcctgcagccgcggacgccgcctggctaggatgacaccacgttgagcgcgcctgcaaacaacaacaacaacaagcgccgccgcggccaccgcgtcctgctccttcgaagcgccgccgccgccggtggagccgcctccgcgcccctggccgtccggagcgcccggccgctggtgtatgtcgcgcctgcccgggacgggctgaagccggcggcggggccggaccgcaggcgccgagcagggcgagggcggccagggcagccgcctgccgccaagccccgagcgccgctgctcgaggaaacgctttcggccgggagctgcggccgccgccagcagttttcatgtttgggattcaggagaatattccgcgcggggggacgaccatgaaggaggagccgctgggcagcggcatgaacccggtgcgctcgtggatgcacacggcgggcgtggtggacgccaacacggccgcccagagcggcgtggggctggcgcgggcgcacttcgagaagcagccgccttccaacctccggaaatccaatttcttccacttcgtgctggcgctctacgataggcaggggcagccggtggagattgaaaggaccgcttttgtggactttgtggagaaagagaaagagccaaacaacgagaaaaccaacaacggcatccactataaactccagttattgtacagcaacggagtcagaacagagcaagatctgtatgttcgcctcatagattcaatgaccaaacaggccatcgtctacgagggccaggacaagaacccggagatgtgccgtgtgctgctgacccacgagatcatgtgcagccggtgctgtgacaagaaaagttgtggcaatagaaacgaaacgccctcagaccctgtaatcattgacagattctttctaaagtttttcctcaagtgcaatcagaactgtttgaagaatgcaggcaaccctcgagatatgcggagattccaggttgttgtatcgacaacagtcaacgtggacggccacgtgctggccgtgtcagacaacatgtttgtgcacaacaattccaaacacgggaggcgggcccgccgcctagacccgtcagaagccactccgtgcatcaaggccatcagtcccagtgaaggctggaccacggggggtgccaccgtcatcataattggcgacaacttctttgacgggctgcaagttgtattcggaactatgttggtgtggagcgagctgataactccccatgccatccgagtccagaccccgccgaggcacattcctggcgtcgtcgaagtgaccctctcctacaaatccaagcagttctgcaaaggtgctcctgggcgctttgtctacaccgcccttaatgaaccaaccatagattacggctttcagaggttgcagaaagtgatcccaagacatccgggtgatcccgaaaggttacccaaggaggtgttactgaagcgggcggcggacctggtggaagccttatacggaatgcctcacaacaaccaggagatcatcttgaagcgagcggcggacatcgccgaggcgctgtacagcgttccccgcaatcacaaccagatccccaccctgggcaacaaccctgcacacacgggcatgatgggcgtcaactccttcagcagccagctagccgtcaacgtgtcagagacgtcacaagccaacgaccaagtcggctacagtcgcaatacaagcagcgtgtccccgcgaggctacgtccccagcagtactccccagcagtccaattacaacacagtcagcactagcatgaatggatatggaagtggcgccatggccagtctaggggtccctggctcgcctggatttcttaatggctcctccgctaactctccctacggcatgaaacagaagagcgccttcgcgcccgtggtccggccccaagcctctcctcctccttcctgcaccagcgccaacgggaatggactgcaagctatgtctgggctggtagtcccgccaatgtgagggacttctgtttaccttccgcagcacccagcatcaaaggacggacttcaggggacacgtttagtatattaagacatgctgatggaaacagtatcttcaaaaaaatcagcagcaattgaaatgctacaaaagactttgtttaaagattttatttaaactattaagaatcaacatgcaaacagcctacttcttcatgaacaattccattttattgactgaacttttctcatattttcacatttctcagtcctgaagaataaggaaaacaaagcgacgcctattttgtataaagtttccgactccgtcttggccatgtctagtaattgctatgtgttgggagaaactttgtgaatgcaccattttgatgatcatgaaacgctgatgaaaaatgcctccaaacatttttctgtactcatacttagattcacaatggttgtgtatctctataatgtgaaatatttttttgtggtgataaaaagagggccaaggaggtatgagccatcagactggaaaaaaggatgactatatgatgaggagaaactggggtggcagggagggagggagggtttatcactgcttaacttcatcttcatgaaatgaaactttgtaacttattgtagttagaaattgtaactttgatattgaattctcttgccttcaacaagcacactgacagagaaaaaatgctactgtctgttggttccaatattctcccactgctagagcttcctgttaagcaagtgtgatctgcaacattttttcaacttttgctagcactgtatactattgcattcttaggctactgtgaggtctatgtttcttgtaccagaaattgtccttttgacttctagatccttcttccctaatgtgttttgtatgtggttataaaattgtagacttttgtgattttgccaaagttgtagctaaatatttatacacttgtcttgaatttttttcagatccacttaaaatatttagaaaaacaagttttattccttatgtgtcttataaggaataaaatggtcttcatttgacacttactttcccatgaacacttgcagttgctaagggactttattttgtaacatatcaattataaatattgtatttatctttgaaattttgtacattgcttttcccaccttttcctttttcttctttcttgtctgtattggtttttgatcacggcctggtgttgtgatactgggaagagcattagccaagaacttgtctctttgatctgtttcgttaagctgaaccagtgtctttacatttcatttgtacttcaaaaaatatgctattgtttagactttccatcctttttttttttattttgaggaaaagtcaaattcattgtttatttttatattatttttaagttatctgaacaaatacttttgaaaaaaaagtttgttgtatagtcaaaacaaatcggtgccacccggccgtgacaaatcctagtagattctgtgcatgtggagcggccgcgaagaggtgacaccgtttggggctgtgtccttattttattttatttttttgtagaatgtaaaaagtcattttagatgccacccattgactttgccacatagctgaactgtgtttactggaaaaattcagaggcctaaagtttaaaataaaatttacttctgatgttttaattaaaatgtttgccacattaacttttctgatgccttaaaagtgaacttctttaaagaacctttgtgctattttatcacaggcttacactacaattgttaataaatacttcatttggagatgtatggtgtaaaacacacaaacacacacaaaaaagcacaagcccgctgcatgacccgtctctcctttctgggactatttctgctgcgtcctgcaccctcctgggcccacctccgattcacagaggtttcagggggacccaaatcactgctggttttcaatttttttttaacaatacatttttgtggtcagttccaacagcactgtccgtacttttaaaactggaatgacctccttcagatatcgtgcctcttagtgccaaacccacagtgagaccaaaagtgtcaggtgtttttttttttttctttctcctttgcactaagtgctttgcagacacggcacagcaaacattttgcaaactgcagcagaaatcgaatttaaaacaaaaaggagggactttaaaataccttcttgacaaaaatcaacaatgcacaacttacaaagtgttcattctagggacaaaattaaataaacagaatgtccccaggagtcagcaggtcacagtctggctttgtgatggttgacaaggtctagctacatgggaaagcctgagaagtcactttggaactaaattgcctccattttattttgtacgagtaagggtttgatctacaaaagagctcacatggacgcactgagaacgcctgccagcttccccatgccctcacttggtttgtgttttaggttaagtagtcaatgcccacatcacttcactgtctcaagactgagcacttcactaaatggtagattttactgttaaagaccctacaataagattgttttatctgtacattttttcagatatttaactgtataaaaatgttcattttacacaatatttaattaaagtatttcttgtctgtgaatttcacttttggtaattttctctgtttttgattattaaaatgactaaacactaa

SEQ ID NO: 227 - protein, 373 aa, Homo sapiens
MASELAMSNSDLPTSPLAMEYVNDFDLMKFEVKKEPVETDRIISQCGRLIAGGSLSSTPMSTPCSSVPPSPSFSAPSPGSGSEQKAHLEDYYWMTGYPQQLNPEALGFSPEDAVEALISNSHQLQGGFDGYARGAQQLAAAAGAGAGASLGGSGEEMGPAAAVVSAVIAAAAAQSGAGPHYHHHHHHAAGHHHHPTAGAPGAAGSAAASAGGAGGAGGGGPASAGGGGGGGGGGGGGGAAGAGGALHPHHAAGGLHFDDRFSDEQLVTMSVRELNRQLRGVSKEEVIRLKQKRRTLKNRGYAQSCRFKRVQQRHVLESEKNQLLQQVDHLKQEISRLVRERDAYKEKYEKLVSSGFRENGSSSDNPSSPEFFM

SEQ ID NO: 228 - DNA, 6900 bp, Homo sapiens
  source 1..6900: mol_type = genomic DNA
cttgctggatcagaggctttaaaatcttttttcatcttctagctgtagctcgggctgcttgtcggcttggcctccccctcccccctttgctctctgcctcgtctttccccaggacttcgctattttgcttttttaaaaaaaggcaagaaagaactaaactcccccctccctctcctccagtcgggctgcacctctgccttgcactttgcacagaggtagagagcgcgcgagggagagagaggaaagaaaaaaaataataaagagagccaagcagaagaggaggcgagaagcatgaagtgttaactcccccgtgccaaggcccgcgccgcccggacagacgcccgccgcgcctccagccccgagcggacgccgcgcgcgccctgcctgcagcccgggccggcgaggcgagcccttccttatgcaaagcgcgcagcggagcggcgagcgggggacgccgcgcaccgggccgggctcctccagcttcgccgccgcagccaccaccgccgccaccgcagctcgcggaggatcttcccgagcctgaagccgccggctcggcgcgcaaggaggcgagcgagcaaggaggggccggggcgagcgagggagcacattggcgtgagcaggggggagggagggcgggcgcggggggcgcgggcagggcgggggggtgtgtgtgtgagcgcgctcggaggtttcgggccagccaccgccgcgcaagctagaagcgccccagcccggcaagctggctcacccgctggccacccagcacagcccgctggcccctctcctgcagcccatctggcggagcggcggcggcggcggcggcggcggcaggagaatggcatcagaactggcaatgagcaactccgacctgcccaccagtcccctggccatggaatatgttaatgacttcgatctgatgaagtttgaagtgaaaaaggaaccggtggagaccgaccgcatcatcagccagtgcggccgtctcatcgccgggggctcgctgtcctccacccccatgagcacgccgtgcagctcggtgcccccttcccccagcttctcggcgcccagcccgggctcgggcagcgagcagaaggcgcacctggaagactactactggatgaccggctacccgcagcagctgaaccccgaggcgctgggcttcagccccgaggacgcggtcgaggcgctcatcagcaacagccaccagctccagggcggcttcgatggctacgcgcgcggggcgcagcagctggccgcggcggccggggccggtgccggcgcctccttgggcggcagcggcgaggagatgggccccgccgccgccgtggtgtccgccgtgatcgccgcggccgccgcgcagagcggcgcgggcccgcactaccaccaccaccaccaccacgccgccggccaccaccaccacccgacggccggcgcgcccggcgccgcgggcagcgcggccgcctcggccggtggcgctgggggcgcgggcggcggtggcccggccagcgctgggggcggcggcggcggcggcggcggcggaggcggcgggggcgcggcgggggcggggggcgccctgcacccgcaccacgccgccggcggcctgcacttcgacgaccgcttctccgacgagcagctggtgaccatgtctgtgcgcgagctgaaccggcagctgcgcggggtcagcaaggaggaggtgatccggctgaagcagaagaggcggaccctgaaaaaccgcggctatgcccagtcctgccgcttcaagagggtgcagcagagacacgtcctggagtcggagaagaaccagctgctgcagcaagtcgaccacctcaagcaggagatctccaggctggtgcgcgagagggacgcgtacaaggagaaatacgagaagttggtgagcagcggcttccgagaaaacggctcgagcagcgacaacccgtcctctcccgagtttttcatgtgagtctgacacgcgattccagctagccaccctgataagtgctccgcgggggtccggctcgggtgtgggcttgctagttctagagccatgctcgccaccacctcaccacccccacccccaccgagtttggcccccttggccccctacacacacacaaacccgcacgcacacaccacacacacacacacacacacacacacaccccacaccctgctcgagtttgtggtggtggtggctgttttaaactggggagggaatgggtgtctggctcatggattgccaatctgaaattctccataacttgctagcttgttttttttttttttttacacccccccgccccacccccggacttgcacaatgttcaatgatctcagcagagttcttcatgtgaaacgttgatcacctttgaagcctgcatcattcacatattttttcttcttcttccccttcagttcatgaactggtgttcattttctgtgtgtgtgtgtgttttattttgtttggatttttttttttaattttacttttagagcttgctgtgttgcccaccttttttccaacctccaccctcactccttctcaacccatctcttccgagatgaaagaaaaaaaaaagcaaagtttttttttcttctcctgagttcttcatgtgagattgagcttgcaaaggaaaaaaaaatgtgaaatgttatagacttgcagcgtgccgagttccatcgggttttttttttagcattgttatgctaaaatagagaaaaaaatcctcatgaaccttccacaatcaagcctgcatcaaccttctgggtgtgacttgtgagttttggccttgtgatgccaaatctgagagtttagtctgccattaaaaaaactcattctcatctcatgcattattatgcttgctactttgtcttagcaacaatgaactataactgtttcaaagactttatggaaaagagacattatattaataaaaaaaaaaagcctgcatgctggacatgtatggtataattattttttcctttttttttccttttggcttggaaatggacgttcgaagacttatagcatggcattcatacttttgttttattgcctcatgacttttttgagtttagaacaaaacagtgcaaccgtagagccttcttcccatgaaattttgcatctgctccaaaactgctttgagttactcagaacttcaacctcccaatgcactgaaggcattccttgtcaaagataccagaatgggttacacatttaacctggcaaacattgaagaactcttaatgttttctttttaataagaatgacgccccactttggggactaaaattgtgctattgccgagaagcagtctaaaatttattttttaaaaagagaaactgccccattatttttggtttgttttatttttattttatattttttggcttttggtcattgtcaaatgtggaatgctctgggtttctagtatataatttaattctagtttttataatctgttagcccagttaaaatgtatgctacagataaaggaatgttatagataaatttgaaagagttaggtctgtttagctgtagattttttaaacgattgatgcactaaattgtttactattgtgatgttaaggggggtagagtttgcaaggggactgtttaaaaaaagtagcttatacagcatgtgcttgcaacttaaatataagttgggtatgtgtagtctttgctataccactgactgtattgaaaaccaaagtattaagaggggaaacgcccctgtttatatctgtaggggtattttacattcaaaaatgtatgtttttttttcttttcaaaattaaagtatttgggactgaattgcactaagatataacctgcaagcatataatacaaaaaaaaattgcaaaactgtttagaacgctaataaaatttatgcagttataaaaatggcattactgcacagttttaagatgatgcagatttttttacagttgtattgtggtgcagaactggattttctgtaacttaaaaaaaaatccacagttttaaaggcaataatcagtaaatgttattttcagggactgacatcctgtctttaaaaagaaatgaaaagtaaatcttaccacaataaatataaaaaaatcttgtcagttacttttcttttacatattttgctgtgcaaaattgttttatatcttgagttactaactaaccacgcgtgttgttcctatgtgcttttctttcattttcaattctggttatatcaagaaaagaataatctacaataataaacggcatttttttttgattctgtactcagtttcttagtgtacagtttaactgggcccaacaacctcgttaaaagtgtaaaatgcatccttttctccagtggaaggattcctggaggaatagggagacagtaattcagggtgaaattataggctgttttttgaagtgaggaggctggccccatatactgattagcaatatttaatatagatgtaaattatgacctcatttttttctccccaaagttttcagttttcaaatgagttgagccataattgcccttggtaggaaaaacaaaacaaaacagtggaactaggcttcctgagcatggccctacacttctgatcaggagcaaagccatccatagacagaggagccggacaaatatggcgcatcagaggtggcttgcgcacatatgcattgaacggtaaagagaaacagcgcttgccttttcactaaagttgactatttttccttcttctcttacacaccgagattttcttgttagcaaggcctgacaagatttaacataaacatgacaaatcatagttgtttgttttgttttgcttttctctttaacactgaagatcatttgtcttaaataggaaaaagaaaatccactccttacttccatatttccaagtacatatctggtttaaactatgttatcaaatcatatttcaccgtgaatattcagtggagaacttctctacctggatgagctagtaatgatttcagatcatgctatccccagaaataaaagcaaaaaataatacctgtgtggaatataggctgtgctttgatttactggtatttaccccaaaataggctgtgtatgggggctgacttaaagatcccttggaaagactcaaaactaccttcactagtaggactcctaagcgctgacctatttttaaatgacacaaattcatgaaactaatgttacaaattcatgcagtttgcactcttagtcatcttcccctagcacaccaatagaatgttagacaaagccagcactgttttgaaaatacagccaaacacgatgacttttgttttgttttctgccgttcttaaaagaaaaaaagataatattgcaactctgactgaaagacttatttttaagaaaacaggttgtgtttggtgctgctaagttctggccagtttatcatctggccttcctgcctattttttacaaaacacgaagacagtgtgtaacctcgacattttgaccttcctttatgtgctagtttagacaggctcctgaatccacacttaattttgcttaacaaaagtcttaatagtaaacctcccctcatgagcttgaagtcaagtgttcttgacttcagatatttctttcctttttttttttttttcctcatcacaactaagagatacacaaactctgaagaagcagaaatggagagaatgcttttaacaaaaaagcatctgatgaaagattttaggcaaacattctcaaaataagagtgatattctggatgtagttattgcagttatctcatgacaaatgaggcctggattggaaggaaaatatagttgtgtagaattaagcattttgataggaatctacaaggtagttgaatataataagcaggtttgggcccccaaactttagaaaatcaaatgcaaaggtgctggcaaaaatgaggtttgagtggctggctgtaagagaaggttaactcctagtaaaaggcatttttagaaataacaattactgaaaactttgaagtatagtgggagtagcaaacaaatacatgttttttttttcttacaaagaactcctaaatcctgagtaagtgccattcattacaataagtctctaaatttaaaaaaaaaaaaatcatatgaggaaatctagctttcccctttacgctgcgtttgatctttgtctaaatagtgttaaaattcctttcattccaattacagaactgagcccactcgcaagttggagccatcagtgggatacgccacattttggaagccccagcatcgtgtacttaccagtgtgttcacaaaatgaaatttgtgtgagagctgtacattaaaaaaaatcatcattattattattatttgcagtcatggagaaccacctacccctgacttctgtttagtctcctttttaaataaaaattactgtgttagagaagaaggctattaaatgtagtagttaactatgcctcttgtctgggggtttcatagagaccggtaggaaagcgcactcctgcttttcgatttatggtgtgtgcaagtaaacaggtgcattgctttcaacctgccatactagttttaaaaattcactgaaattacaaagatacatatatatgcatatatataatggaaagtttcccggaatgcaacaattagcattttaaaatcatatataggcatgcacattctaaatagtactttttcatgcttcattgtttctctggcagataattttactaagaagaaaaatagatattcgactccccttccctaaacaaatccacgggcagaggctccagcggagccgagccccctggttttctcgtaggccctagacggtgttgcatttatcagtgatgtcaaacgtgctcatttgtcagacatagctgtaaatgaaaacaatgtgtggcaaaatacaaagttagttaaataca

SEQ ID NO: 229 - protein, 100 aa, Homo sapiens
MATFPPATSAPQQPPGPEDEDSSLDESDLYSLAHSYLGPLIMPMPTSPLTPALVTGGGGRKGRTKREAAANTNRPSPGGHERKLVTKLQNSERKKRGARR

SEQ ID NO: 230 - DNA, 5345 bp, Homo sapiens
  source 1..5345: mol_type = genomic DNA
gccaggaaagcagagacagacaaagcgttaggagaagaagagaggcagggaagacaagccaggcacgatggccaccttcccaccagcaaccagcgccccccagcagcccccaggcccggaggacgaggactccagcctggatgaatctgacctctatagcctggcccattcctacctcgggcctctcatcatgcctatgcccacttcacctctgactcctgccttggttacaggaggtggaggccggaaaggtcgcaccaagagagaagctgctgccaacaccaaccgccccagccctggcgggcacgagaggaaactggtgaccaagctgcagaattcagagaggaagaagcgaggggcacggcgctgagacagagctggagatgaggccagaccatggacactacacccagcaatagagacgggactgcggaggaaggaggacccaggacaggatccaggccggcttgccacaccccccacccctaggacttattcccgctgactgagtctctgaggggctaccaggaaagcgcctccaaccctagcaaaagtgcaagatggggagtgagaggctgggaatggaggggcagagccaggaagatcccccagaaaagaaagctacagaagaaactggggctcctccagggtggcagcaacaataaatagacacgcacggcagccacagcttgggtgtgtgttcatccttgttctttgtgtgtttttgttcgggcatgtgtgtgcttgcctgtgcctgcacattcatgagcctgagagagcatctttgatgtgtatttgtgtttggtgtatgtatctggggcagggagtgttcctgctcttgcagggtctacgctgtgaatgcagcttttggtttgtttgcttttgcttgcatatattttagtgcatacatttctgtgggctcctacgtagtggaaaggaatttcttctgcttttttgcgatactgcccatgaaacacggccctccccagcacctgtttttgttgattgtgtcctgttcatagacgggaacgctacttatgagtgccatctaaaagtcagagaaaactgagatttaaaatattaaaagccagggccgggggcagtggctcacgcctgtaatcccagcactttgggaggccgaggtgggtggatcacaaagtcaggagttcaagaccagcctgaccaacatggtgaaaccccatctccactaaaaataccaaaaattagccaagcatggtggcaggtgcctgtaatcccagctgctcaggaggctgaggcagtagaatcgcttgaacccaggaggtagaggttgcagtgagccgacatcgtgccattgcactccagcctgggtgacagagggagactctgtctcaaaacaaacaaacaaacaaaaactaaagtctgggagcagtggctcatgcctgtaatcacagcagtttaggaggccgaagtgggaggattacttgagcctaggagtttgagaccagcctgagcatcatagtaagaccccatctctacaattttttttttgagacagagtctcactctgttgctcaggttagagtgcagtggcaccatcttggctcactgcaacctctgcctcccgggttcaagcaattctcgtgcctacgccccctgagtagctgggattacaggtgagcaccaccacgcctggctaatttttgtttttttgtttttttgatacagagtctcactctgttgctcaggctggagtgcagtggcatgatcgcagctcactgcaacctccgcctcctgggttcaagctattctcctgcttcagcctcctgagtagctgggactacaggcacctgccaccatgcctggctaatttttgtatttttagtagaaacagagtttcaccatgttggccaggatggtctcaatctcttgacctcatgatccacccaccttggcctcccaaagtgctgggattacaggcgtcagccaccacgcctggccaatttttgtatttttagtagagatggggtttcatcatgttggccaggctggtctcaaactcctggcctcaaatgatctgcccacctcagccttccagcctttgggaggccaaggagggaggatcgcttgaggccaggagttcgagaccagcctaggcaacataccaaggccctgtctctacaaaaatttaaaaattagcaaagcatggtggctcatgcctgtagtcctagttgctcagaggctgaagttggaggatcccttgaacccagttggaggctacagtaagccatgatggtgccactgcactgcagcctgagcaatagagtgaaaccatgtattgaaaaagaaagaaagaaagaaagagaaaaagaaggaaggaaggaaaagaaaggaaggaagaaagaaaagaaagagagaaaggagagaaaaagaagaaagagagagaaagagaagaaagaaagaaagaaagaaagaaagaaagaaagaaagaaagaaagaaaggaaggaaggaaagaaagaaagaaagaaggaaagaaagaaagaaagaaagaaagaaagaaagaaagaaaagagcgagccccggcacagtggctcacacctgtaatcccagcactttgggaggccaaggcaggcggatcacctgaggtcagtagttcgagttcgaggcagaagttgtggtgagccaagatcacgccattgcactccagcctgggcaacaagagcaaaactccatctcaaaaaaagaaaaagagagagaaagaaagagagaaggagggaaagaaagaggaaggaaaggagggagggaaggagggaaggaggaaaggagggaaggaaatattaagggatttgccctagctcactcagggctcatattcagggatcttattgtcttcagatatgaggtgggtccccaggccaagggggctgtgggacagggttctgggaagtgagatgggggagggagattcttattcagtgctatgtctcaggaccagagttgaagaaaaatatggggcaaacaggaagtgtggttggagctgagattatttgttgattgagatacaggtttgggttgctattatgaagaatcaaaacaggatacaaagttactcctctcttacttagccccaagtcctcctgggttcaaatggcagctccattgtgttgaggattccagctatttttactcactgttctgccacacccaaaacatggcttccactttgaggcccaaaatagctactccaatacctgccatcacgtttgcatctcagcctgtggaaagagtgtggaagagacagaggaggccacacctcttcaattttagggcataatctaaaagtagaacacatcacatctgctccgaatgtggctacatggccacacccaactacgagagaggttgggaaatgtagcattgagctgcttagccacgtggtagccaaaacttgggcaacagcagggttttatgatgaaagggaaccagagagagtgaatacacgaagaagctagcaagaaacattgcccacatgcagactcaacagatgtcgggggagaaaactctgagattgggccccgggcagagcctgtttccttttcccctgtccctttcacataagccccacactgcagaacttggagtcagtggttcatggggacccaaggacatccacagaagaccgaagtacctgatagagcaggaacagatgcacgtcagactacgaccagcttgaacccttggggacctgtatccatccccttcccttgagtccagcctaatcacagccacagaatgaatccatgacccggaaggacatttaaggacctctggcccaactctccccacgcaacagtgggattgcctctgcttgtttgaataccactatggagggggaacttgctacatttaaaggttgctgaatttttttgttttgttttgggagatggagtctctctctgtcgcccaggctggagtgcagtggtgtgatctcgacccactgcaacctccgcttcctgggttcaagcgattttcatgcctcagcctccctagtagctgggactacaggtgtgggccaccacacctggctaatttttgtatttttagtagagacggggtttcaccatgttgcccaggctggtctcaaactcccgacctcaggtgatctgcctgccttggcctcccaaagtgttgggattacaggcttgagccacaccacccggcaaaggttgcctaattctatctcagggcaagtctgttcatgtgggctgaaatctgtctctcatacctgcccttaaataagcttctgtttttctggtcctttctgtctgagatgactttaattcctcccccgaccccatctcatgaggctcagcttagatgtttccttctcttcctccctgatggctgcgtgcagaagctcatacctgcaatcccagcactttgggaggccgaggtgggaggattgcttgggcccaggagttctaggccagcctagcaacacagcaagaccctctctctacaaaaaattaaaaattagccaggtgtggtggcttgtgcctgtgggcccacctactcaggaggctgaggtgagaggatctcttgagtctgggaggtggaggctgcagtgagccatgatctcactccagcctgggcgtcagagtgagacctgtctcaaaaaaacaaaaaccaaagttttccttaacttgcaatcctccacccatctcctgacctccaccccaggccgaccctcctctgggatcacacggaagcctgggcccagaatcttacagtctaccttggaaatccttcagattttgcctccaactcctctttccatacaagttacatattaactcaatcaatgcttattggatgccttctgagttaccaggatgctgctggaggcctgtgctgctgagagggacaaatagaatcctgatatgactggaaccaggaatagggccaccatcagctctagaacccagggagcaaatggcaggagcaacaccaggcagtcggccctcagcccagcacagacctggtgtgttggcttcagaaaatcctggaaacatctctgctgtgtttggagctcctattctgtgagaagtgttttataggctttgctccatttaatccttacgataacccaataggacatactattaatgatcccctttttttcagaccggaaaactgaggttcggagacattaagtaatttgcctaaagtcacacagccacatcctgtaggacttggaattatgaactcaggtctatctgaattttaaagcctggatttttccccctttgctacatgcctgggaagaaccatgtattgacaatgaagaatcctaaactctcgttaa

SEQ ID NO: 231 - protein, 202 aa, Homo sapiens
MMQDVSSSPVSPADDSLSNSEEEPDRQQPPSGKRGGRKRRSSRRSAGGGAGPGGAAGGGVGGGDEPGSPAQGKRGKKSAGCGGGGGAGGGGGSSSGGGSPQSYEELQTQRVMANVRERQRTQSLNEAFAALRKIIPTLPSDKLSKIQTLKLAARYIDFLYQVLQSDELDSKMASCSYVAHERLSYAFSVWRMEGAWSMSASH

SEQ ID NO: 232 - DNA, 1629 bp, Homo sapiens
  source 1..1629: mol_type = genomic DNA
aactcccagacacctcgcgggctctgcagcaccggcaccgtttccaggaggcctggcggggtgtgcgtccagccgttgggcgctttctttttggacctcggggccatccacaccgtcccctccccctcccgcctccctccccgcctcccccgcgcgccctccccgcggaggtccctcccgtccgtcctcctgctctctcctccgcgggccgcatcgcccgggccggcgccgcgcgcgggggaagctggcgggctgaggcgccccgctcttctcctctgccccgggcccgcgaggccacgcgtcgccgctcgagagatgatgcaggacgtgtccagctcgccagtctcgccggccgacgacagcctgagcaacagcgaggaagagccagaccggcagcagccgccgagcggcaagcgcgggggacgcaagcggcgcagcagcaggcgcagcgcgggcggcggcgcggggcccggcggagccgcgggtgggggcgtcggaggcggcgacgagccgggcagcccggcccagggcaagcgcggcaagaagtctgcgggctgtggggcggcggcggcgcgggcggcggcggcggcagcagcagcggcggcgggagtccgcagtcttacgaggagctgcagacgcagcgggtcatggccaacgtgcgggagcgccagcgcacccagtcgctgaacgaggcgttcgccgcgctgcggaagatcatccccacgctgccctcggacaagctgagcaagattcagaccctcaagctggcggccaggtacatcgacttcctctaccaggtcctccagagcgacgagctggactccaagatggcaagctgcagctatgtggctcacgagcggctcagctacgccttctcggtctggaggatggagggggcctggtccatgtccgcgtcccactagcaggcggagccccccaccccctcagcagggccggagacctagatgtcattgtttccagagaaggagaaaatggacagtctagagactctggagctggataactaaaaataaaaatatatgccaaagattttcttggaaattagaagagcaaaatccaaattcaaagaaacagggcgtggggcgcacttttaaaagagaaagcgagacaggcccgtggacagtgattcccagacgggcagcggcaccatcctcacacctctgcattctgatagaagtctgaacagttgtttgtgttttttttttttttttttttgacgaagaatgtttttatttttatttttttcatgcatgcattctcaagaggtcgtgccaatcagccactgaaaggaaaggcatcactatggactttctctattttaaaatggtaacaatcagaggaactataagaacacctttagaaataaaaatactgggatcaaactggcctgcaaaaccatagtcagttaattctttttttcatccttcctctgaggggaaaaacaaaaaaaaacttaaaatacaaaaaacaacattctatttatttattgaggacccatggtaaaatgcaaatagatccggtgtctaaatgcattcatatttttatgattgttttgtaaatatctttgtatatttttctgcaataaataaatataaaaaatttagagaa

SEQ ID NO: 233 - protein, 160 aa, Homo sapiens
MEEGSSSPVSPVDSLGTSEEELERQPKRFGRKRRYSKKSSEDGSPTPGKRGKKGSPSAQSFEELQSQRILANVRERQRTQSLNEAFAALRKIIPTLPSDKLSKIQTLKLAARYIDFLYQVLQSDEMDNKMTSCSYVAHERLSYAFSVWRMEGAWSMSASH

SEQ ID NO: 234 - DNA, 1342 bp, Homo sapiens
  source 1..1342: mol_type = genomic DNA
gcacaagccggccttgaaatcagagcctttccagcaactccgagagcgtgtgctcggcgaccgcgggcttggccagcggcgcgcgctcggcgccccggcgcccccagccccacgcgcgccgggcgggcgccatggaggagggctccagctcgcccgtgtcccccgtggacagcctgggcaccagcgaggaggagctcgagaggcagcccaagcgcttcggccggaagcggcgctacagcaagaagtcgagcgaagatggcagcccgaccccgggcaagcgcggcaagaagggcagccccagcgcgcagtccttcgaggagctgcagagccagcgcatcctggccaacgtgcgcgagcgccagcgcacccagtcgctcaacgaggccttcgcggcgctgcgcaagatcatccccacgctgccctctgacaagctgagcaagatccagacgctcaagctggccgccaggtacatagacttcctctaccaggtcctgcagagcgacgagatggacaataagatgaccagctgcagctacgtggcccacgagcgcctcagctacgccttctccgtgtggcgcatggagggcgcgtggtccatgtccgcctcccactagcgccgcgccacccacctccggaccggcgcgccagggctgtccgtcgcgtcggcggcgcaagtggaattgggatgcattcgagtctgtaacttctgaaacctgaacaacctcaggaggcccccacctctgccctccaccagcgtcgagagaagggacagcagtgacatcggacagaagacccgggctcccgtcctcccccaggacggtccccacataggaagggcactcccagccctcttgctggtgacattgtcatggtcatcttgtttctgtttggatttttcttctgggtcttatgtttggggggaggtttattctttctgaaaatgtctagattcaggaacacatttatgaggatttggattttgaatttgtatttccctctaagtgccttttttaatgtctatttttttaataaaacagaaatgcattcttgtacaattctgttgaaactggaccaaggctctcagaagaggacccccgagttccttcccctcccccgagcctctgcatgattgtttcaagtcagcctggaattcttactttcacgccgctattcttttcctttctccgtgattgcttggctagccatttaaaaaaaaatattctctgttcagtgtatatgttgcttgtttgttttatttattgagatatttttacaagctaagtgactgcagtgtggctgtgtatcctgctccccacccaggaaaaataaagacgtccgcgca

SEQ ID NO: 235 - protein, 778 aa, Homo sapiens
MAQKMDCGAGLLGFQAEASVEDSALLMQTLMEAIQISEAPPTNQATAAASPQSSQPPTANEMADIQVSAAAARPKSAFKVQNATTKGPNGVYDFSQAHNAKDVPNTQPKAAFKSQNATPKGPNAAYDFSQAATTGELAANKSEMAFKAQNATTKVGPNATYNFSQSLNANDLANSRPKTPFKAWNDTTKAPTADTQTQNVNQAKMATSQADIETDPGISEPDGATAQTSADGSQAQNLESRTIIRGKRTRKINNLNVEENSSGDQRRAPLAAGTWRSAPVPVTTQNPPGAPPNVLWQTPLAWQNPSGWQNQTARQTPPARQSPPARQTPPAWQNPVAWQNPVIWPNPVIWQNPVIWPNPIVWPGPVVWPNPLAWQNPPGWQTPPGWQTPPGWQGPPDWQGPPDWPLPPDWPLPPDWPLPTDWPLPPDWIPADWPIPPDWQNLRPSPNLRPSPNSRASQNPGAAQPRDVALLQERANKLVKYLMLKDYTKVPIKRSEMLRDIIREYTDVYPEIIERACFVLEKKFGIQLKEIDKEEHLYILISTPESLAGILGTTKDTPKLGLLLVILGVIFMNGNRASEAVLWEALRKMGLRPGVRHPLLGDLRKLLTYEFVKQKYLDYRRVPNSNPPEYEFLWGLRSYHETSKMKVLRFIAEVQKRDPRDWTAQFMEAADEALDALDAAAAEAEARAEARTRMGIGDEAVSGPWSWDDIEFELLTWDEEGDFGDPWSRIPFTFWARYHQNARSRFPQTFAGPIIGPGGTASANFAANFGAIGFFWVE

SEQ ID NO: 236 - DNA, 2632 bp, Homo sapiens
  source 1..2632: mol_type = genomic DNA
agcacttccggtcacgccatcgttgctgccttcttcgtcaagcccccaggctccgctcttgccagagggacaggagccatggctcagaaaatggactgtggtgcgggcctcctcggcttccaggctgaggcctccgtagaagacagcgccttgcttatgcagaccttgatggaggccatccagatctcagaggctccacctactaaccaggccaccgcagctgctagtccccagagttcacagcccccaactgccaatgagatggctgacattcaggtttcagcagctgccgctaggcctaagtcagcctttaaagtccagaatgccaccacaaaaggcccaaatggtgtctatgatttctctcaggctcataatgccaaggatgtgcccaacacgcagcccaaggcagcctttaagtcccaaaatgctaccccaaagggtccaaatgctgcctatgatttttcccaggcagcaaccactggtgagttagctgctaacaagtctgagatggccttcaaggcccagaatgccactactaaagtgggcccaaatgccacctacaatttctctcagtctctcaatgccaatgacctggccaacagcaggcctaagacccctttcaaggcttggaatgataccactaaggccccaacagctgatacccagacccagaatgtaaatcaggccaaaatggccacttcccaggctgacatagagaccgacccaggtatctctgaacctgacggtgcaactgcacagacatcagcagatggttcccaggctcagaatctggagtcccggacaataattcggggcaagaggacccgcaagattaataacttgaatgttgaagagaacagcagtggggatcagaggcgggccccactggctgcagggacctggaggtctgcaccagttccagtgaccactcagaacccacctggcgcaccccccaatgtgctctggcagacgccattggcttggcagaacccctcaggctggcaaaaccagacagccaggcagaccccaccagcacgtcagagccctccagctaggcagaccccaccagcctggcagaacccagtcgcttggcagaacccagtgatttggccaaacccagtaatctggcagaacccagtgatctggccaaaccccattgtctggcccggccctgttgtctggccgaatccactggcctggcagaatccacctggatggcagactccacctggatggcagaccccaccgggctggcagggtcctccagactggcaaggtcctcctgactggccgctaccacccgactggccactgccacctgattggccacttcccactgactggccactaccacctgactggatccccgctgattggccaattccacctgactggcagaacctgcgcccctcgcctaacctgcgcccttctcccaactcgcgtgcctcacagaacccaggtgctgcacagccccgagatgtggcccttcttcaggaaagagcaaataagttggtcaagtacttgatgcttaaggactacacaaaggtgcccatcaagcgctcagaaatgctgagagatatcatccgtgaatacactgatgtttatccagaaatcattgaacgtgcatgctttgtcctagagaagaaatttgggattcaactgaaagaaattgacaaagaagaacacctgtatattctcatcagtacccccgagtccctggctggcatactgggaacgaccaaagacacacccaagctcggtctcctcttggtgattctgggtgtcatcttcatgaatggcaaccgtgccagtgaggctgtcctctgggaggcactacgcaagatgggactgcgtcctggggtgagacatcccctccttggagatctaaggaaacttctcacctatgagtttgtaaagcagaaatacctggactacagacgagtgcccaacagcaaccccccggagtatgagttcctctggggcctccgttcctaccatgagactagcaagatgaaagtgctgagattcattgcagaggttcagaaaagagaccctcgtgactggactgcacagttcatggaggctgcagatgaggccttggatgctctggatgctgctgcagctgaggccgaagcccgggctgaagcaagaacccgcatgggaattggagatgaggctgtgtctgggccctggagctgggatgacattgagtttgagctgctgacctgggatgaggaaggagattttggagatccctggtccagaattccatttaccttctgggccagataccaccagaatgcccgctccagattccctcagacctttgccggtcccattattggtcctggtggtacagccagtgccaacttcgctgccaactttggtgccattggtttcttctgggttgagtgagatgttggatattgctatcaatcgcagtagtctttcccctgtgtgaggctgaagcctcagattccttctaaacacagctatctagagagccacatcctgttgactgaaagtggcatgcaagataaatttatttgctgttccttgtctactgctttttttccccttgtgtgctgtcaagttttggtatcagaaataaacattgaaattgcaaagtgaa

SEQ ID NO: 237 - protein, 733 aa, Homo sapiens
MERRSESPCLRDSPDRRSGSPDVKGPPPVKVARLEQNGSPMGARGRPNGAVAKAVGGLMIPVFCVVEQLDGSLEYDNREEHAEFVLVRKDVLFSQLVETALLALGYSHSSAAQAQGIIKLGRWNPLPLSYVTDAPDATVADMLQDVYHVVTLKIQLQSCSKLEDLPAEQWNHATVRNALKELLKEMNQSTLAKECPLSQSMISSIVNSTYYANVSATKCQEFGRWYKKYKKIKVERVERENLSDYCVLGQRPMHLPNMNQLASLGKTNEQSPHSQIHHSTPIRNQVPALQPIMSPGLLSPQLSPQLVRQQIAMAHLINQQIAVSRLLAHQHPQAINQQFLNHPPIPRAVKPEPTNSSVEVSPDIYQQVRDELKRASVSQAVFARVAFNRTQGLLSEILRKEEDPRTASQSLLVNLRAMQNFLNLPEVERDRIYQDERERSMNPNVSMVSSASSSPSSSRTPQAKTSTPTTDLPIKVDGANINITAAIYDEIQQEMKRAKVSQALFAKVAANKSQGWLCELLRWKENPSPENRTLWENLCTIRRFLNLPQHERDVIYEEESRHHHSERMQHVVQLPPEPVQVLHRQQSQPAKESSPPREEAPPPPPPTEDSCAKKPRSRTKISLEALGILQSFIHDVGLYPDQEAIHTLSAQLDLPKHTIIKFFQNQRYHVKHHGKLKEHLGSAVDVAEYKDEELLTESEENDSEEGSEEMYKVEAEEENADKSKAAPAEIDQR

SEQ ID NO: 238 - DNA, 5567 bp, Homo sapiens
  source 1..5567: mol_type = genomic DNA
gagtccggctctggctgctggcagaggcggccgagaggggagaggctggaggtgacagcttgggcggccgccgcgtttcctcccgcgcgcggtcccgggtccctgcgtcttctcggctcttggtgttaccggtcccaccgctctggccgcgcctcctcgcgagctagccgccctgcgaaccagcagccccggctcgccgccgccgccgccgcctccgggttctcagccctttctctccagaacgggtctccttcccgaaggtgtgaaaaggctctttcagcctccttctcttcccccctcctccgccgtcccctcccccgctcgctcgggtgtccctttggaggagtcctttccctctcctcctcctccccctcctccctccccccatcatcatcataacaaccatctccgcaccagaagaagacaccctgacccaggaccttaaacattaggacctggggaagagggaaggggaaggagtaaagaggaagactaggagaacactgcaaagccaagcaccagaaactttccaccctggattctctacttttgctccatggacagagccccagtcagccaagtttcagacagaccgtgagcagtccctgtgcgttttattgcgacctgccggtgggaactttgtctccgagtcggagcagcatggagcggcggagcgagagcccgtgtctgcgggacagccccgaccggcggagcggcagcccggacgtcaaggggcctcccccagtgaaggtggcccggctggagcagaacggcagccccatgggagcccgcgggaggcccaacggcgccgtggccaaggccgtgggaggtttgatgattcctgtcttttgtgtcgtggagcagttggacggctctcttgaatatgacaacagagaagaacacgccgagtttgtcctggtgcggaaagatgtgctttttagccagctggtggagactgcgctcctggccctggggtattctcacagctctgcggcccaggcccaaggaataatcaagctgggaaggtggaaccctctccccctcagttatgtgacagatgcacccgacgcgacagtggccgacatgctacaagatgtctatcatgttgtgacgttgaaaatccaattacaaagttgttcaaagttggaagacttgcctgcggagcagtggaaccatgccacagtccgcaatgccttaaaggaactgctcaaagagatgaaccagagcacattagccaaagaatgccctctctcccagagtatgatttcatccattgtaaatagcacatattatgccaatgtgtcagcaaccaagtgccaggagtttgggagatggtataaaaagtacaagaagattaaagtggaaagagtggaacgagaaaacctttcagactattgtgttctgggccagcgtccaatgcatttaccaaatatgaaccagctggcatccctggggaaaaccaacgaacagtctcctcacagccaaattcaccacagtactccaatccgaaaccaagtgcccgcattacagcccatcatgagccctggtcttctttctccccagcttagtccacaacttgtaaggcaacaaatagccatggcccatctgataaaccaacagattgccgttagccggctcctggctcaccagcatcctcaagccatcaaccagcagttcctgaaccatccacccatccccagagcagttaagccagagccaaccaactcttccgtggaagtctctccagatatctaccagcaagtcagagatgagctgaagagggccagtgtgtcccaagctgtctttgcaagagtggcattcaaccgcacacagggattgttgtctgagattctgcgtaaggaagaagaccctcggacagcctctcagtctcttctagtaaacctgagggccatgcagaatttcctcaatctgccagaagtggagcgagatcgcatctaccaggatgagagggagcggagcatgaatcccaatgtgagcatggtctcctcggcctccagcagtcccagctcctcccgaacccctcaggccaaaacctcgacaccgacaacagacctccctattaaggtggacggcgccaacatcaacatcacagctgccatttatgacgagatccaacaggagatgaaaagggccaaggtgtctcaagccctgtttgccaaagtggctgcaaataaaagtcagggctggctgtgtgaactgctccgctggaaggagaacccaagcccagaaaaccgcaccctctgggaaaacctctgtaccatccgtcgcttcctgaaccttccccagcatgagagggatgtcatctatgaggaggagtcaaggcatcaccacagcgaacgcatgcaacacgtggtccagcttccccctgagccggtgcaggtacttcatagacagcagtctcagccagccaaggagagttcccctcccagagaagaagcgcctcccccacctcctccgactgaagacagttgtgccaaaaagccccggtctcgcacaaagatctccttagaagccctggggatcctccaaagctttattcatgatgtaggcctgtacccagaccaggaagccatccacactctttcggctcagctggatctccccaaacacaccatcatcaagttcttccagaaccagcggtaccacgtgaagcaccacgggaagctgaaagagcacctgggctccgcggtggacgtggctgaatataaggacgaggagctgctgaccgagtcagaggagaacgacagcgaggaaggctccgaggagatgtacaaagtggaggctgaggaggaaaatgctgacaaaagcaaggcagcacctgccgaaattgaccagagataatgtgaacttctactaggcaaagcaatacatcggtccaaggattttctgctttcatttctttaaaagttttttgttagtttgttttttgtttttgtttttgggtttttttggctttatttttgtctttttatgtctgttttgtttttcttacccttttggacatttctttgttgcacaggatacacctatagactgaataagttcagtatttccgaatcagacatcgccttggcaaagacactaaagcgttacactttatcccgtctctatgactggatcatagtcattataatcacaggagactctgccttcattatccttgcacttaacggaagttacatcaggcaagtaccaggatgaaaagaactatgaaataaatgaaggaagctacaagtgtgtgtgtatatgtatatgtatatatctctatatttacatatatatattaaaattgcatgggacagagactttgcaatccgaaagaatagactgtgaaatgagttcttaaagaaaagacttgtttatgtattaaaaaaaccacttcacagtgagtcgctttggctttttgataaactgcggcctgctctcagggtggggtgactatttttgaattcctatttattttttgtgtttgtccctgattttttttttttaattctatggcttcctatctggcagcttaatgggtaatttttgaggtatgtatttaacaaaataaacgacactgccgaaaaaaaaaaaagtgaagtgaaaacaatcagggcacattaaaatgatacaagtcaaataaatcttaaagacacaatgcacacttaaaatgactcaataaaatgacttgctacgttccgttattcaatttgtcattactgtagtgaacagatgcatttctgtggaattccaaataagtaaaactgaaattcagtgcagagaaaactttgtccactagtgcaagtcttgatcaaatgacattttgacattggacatatggaattcatagtatgagccacattttgttgtgaaatttatttacctgcttgtggcttcaaatctgaaaattaataagcctgctcgtttaaaagttgtttgttgttgctgtttttttgtctttttgttttttactagaaaatagttcagtgtaatattaagttagaaaagaagttgctgcccagttaaaggggctccctctcaaataaatctccatccttccctctcccaaaagacatttctgatttctgcttcactttgggcttcctcttcttcgtacacattccatctacctaatcaaacattttcagtccctgatctctcctgtcccttttcctgggatgacagccctaacaagaactgtttttgaatcgttgtgcagctccaggcaatagagtatgtgaagcgatttcagtagaatcacttactcatcctaaaagaaaacattatcccagttacctacatcgcaattaccttatgtaaagcagaactaatgctgactggatgtttaatgggatgagcattaaagctgcaatctactatagtactccagatctctttcggcttcctatgagaaacaccagaagcattactttccacttctacttacagtaattgcaagaggagacctcacattcaggactgcctagtgaacgtaatccatgctttaaactggccattaaacagtcccacatggttggattttttttttttttttgagttgtgctttcacaaaaccttgtcaaagacctcatgcaatatcactttgaaagttattttctgtttactacacaaacattgtaatataactgttaatactatttatatatttgaaaggtataaaaggtaggagttaaaaaaaaaacctctatgtgtagatattaactcagaacttacaatatacagggagaagacatgttgcaatacaagctaattctagctgctcagtaacctctggagtttttaaagggacattttcctgtactttttcaaataatgatgtttaaaaattatcttgacataagcgtcatatacctttgcaaaaggatggttgtttgcagttagccctggccccatccttcctatttctgtagtatgctgcagctttaatcagaaagtccatggttgctgcttcctgatctccgagttactctttccaaattgtcttcttacactgttgctgaaggtcactctgtacacgtaatggaaactgattttgccaagctcttacaaggtggttcatctatcgatggcatccgcatttggtatcttttacacttcaaccaaaaatttattaggtatttttcaatgctaagtcttgccttttattttttaatttcactgccaagtttgcagtggttctaagtgaatctgtgggcattttagcctgtggtcttgccagatctttgcgaattacaatgcatatatgtctatttattcaatatctgtcatataatatctatttggaagaagaaactttctcttgtagtgcctcttgacaaagcacaatttcccgcctttttttttttttgtgaaatgaaaaaaacaaattgtgttttattgcggtatcaacaatgtgaataaggattaacatattgtaaatgttcttttttccatgtaaatcaactatctttgttatcactaagtgataattaatttttaacttatgtgcattgttaggctgttagaattttttggttgttaaaataaacgcattcaataaa

SEQ ID NO: 239 - protein, 457 aa, Homo sapiens
MDSFKVVLEGPAPWGFRLQGGKDFNVPLSISRLTPGGKAAQAGVAVGDWVLSIDGENAGSLTHIEAQNKIRACGERLSLGLSRAQPVQSKPQKASAPAADPPRYTFAPSVSLNKTARPFGAPPPADSAPQQNGQPLRPLVPDASKQRLMENTEDWRPRPGTGQSRSFRILAHLTGTEFMQDPDEEHLKKSSQVPRTEAPAPASSTPQEPWPGPTAPSPTSRPPWAVDPAFAERYAPDKTSTVLTRHSQPATPTPLQSRTSIVQAAAGGVPGGGSNNGKTPVCHQCHKVIRGRYLVALGHAYHPEEFVCSQCGKVLEEGGFFEEKGAIFCPPCYDVRYAPSCAKCKKKITGEIMHALKMTWHVHCFTCAACKTPIRNRAFYMEEGVPYCERDYEKMFGTKCHGCDFKIDAGDRFLEALGFSWHDTCFVCAICQINLEGKTFYSKKDRPLCKSHAFSHV

SEQ ID NO: 240 - DNA, 1712 bp, Homo sapiens
  source 1..1712: mol_type = genomic DNA
gtcagaacactggcggccgatcccaacgaggctccctggagcccgacgcagagcagcgccctggccgggccaagcaggagccggcatcatggattccttcaaagtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcagcagaatggacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgctgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctggggctccctgggccctgccccacccaccttatcctcccaccccactccctccaccaccacagcacaccggtgctggccacaccagccccctttcacctccagtgccacaataaacctgtacccagctgtg

SEQ ID NO: 241 - protein, 190 aa, Homo sapiens
MGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 242 - DNA, 1589 bp, Homo sapiens
  source 1..1589: mol_type = genomic DNA
ggaaaaaaggaaagtgcacttggaagagatccaagtgggcaacttgaagaacaagtgccaaatagcacttctgtcatgctggatgtcagggctctttgtccactttgtatagccgctggcttatagaaggtgctcgataaatctcttgaatttaaaaatcaattaggatgcctctatagtgaaaaagatacagtaaagatgagggataatcaatttaaaaaatgagtaagtacacacaaagcactttatccattcttatgacacctgttacttttttgctgtgtttgtgtgtatgcatgccatgttatagtttgtgggaccctcaaagcaagctggggagagtatatactgaatttagcttctgagacatgatgctcttcctttttaattaacccagaacttagcagcttatctatttctctaatctcaaaacatccttaaactgggggtgatacttgagtgagagaattttgcaggtattaaatgaactatcttcttttttttttttctttgagacagagtcttgctctgtcacccaggctggagtgcagtggcgtgatctcagctcactgcaacctccgcctcccgggttcaagtgattctcctgcctcagcctcctgagtagctgggattacagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 243 - protein, 513 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQGQSRGFVARAGEPCVCWPHFGTHGMMLTPPSSPLELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 244 - DNA, 3038 bp, Homo sapiens
  source 1..3038: mol_type = genomic DNA
ggcagtttcccgaccagagagaacgaacgtgtctgcgggcgcgcggggagcagaggcggtggcgggcggcggcggcaccgggagccgccgagtgaccctcccccgcccctctggccccccaccctcccacccgcccgtggcccgcgcccatggccgcgcgcgctccacacaactcaccggagtccgcgccttgcgccgccgaccagttcgcagctccgcgccacggcagccagtctcacctggcggcaccgcccgcccaccgccccggccacagcccctgcgcccacggcagcactcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaaggtcagtcccggggatttgtagctcgggctggggagccctgtgtgtgctggccccacttcgggacacacgggatgatgctcaccccaccttcttcacccctagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctgaa

SEQ ID NO: 245 - protein, 453 aa, Homo sapiens
MDFFRVVENQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 246 - DNA, 4515 bp, Homo sapiens
  source 1..4515: mol_type = genomic DNA
ggagtttattcataacgcgctctccaagtatacgtggcaatgcgttgctgggttattttaatcattctaggcatcgttttcctccttatgcctctatcattcctccctatctacactaacatcccacgctctgaacgcgcgcccattaatacccttctttcctccactctccctgggactcttgatcaaagcgcggccctttccccagccttagcgaggcgccctgcagcctggtacgcgcgtggcgtggcggtgggcgcgcagtgcgttctcggtgtggagggcagctgttccgcctgcgatgatttatactcacaggacaaggatgcggtttgtcaaacagtactgctacggaggagcagcagagaaagggagagggtttgagagggagcaaaagaaaatggtaggcgcgcgtagttaattcatgcggctctcttactctgtttacatcctagagctagagtgctcggctgcccggctgagtctcctccccaccttccccaccctccccaccctccccataagcgcccctcccgggttcccaaagcagagggcgtgggggaaaagaaaaaagatcctctctcgctaatctccgcccaccggccctttataatgcgagggtctggacggctgaggacccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaagttgtgaatgttttgtttcgtttcttccccctcccaaccaccaccatccctgtttgttttcatcaattgccccttcagagggtggtcttaagaaaggcaagagttttcctctgttgaaatgggtctgggggccttaaggtctttaagttcttggaggttctaagatgcttcctggagactatgataacagccagagttgacagttagaaggaatggcagaaggcaggtgagaaggtgagaggtaggcaaaggagatacaagaggtcaaaggtagcagttaagtacacaaagaggcataaggactggggagttgggaggaaggtgaggaagaaactcctgttactttagttaaccagtgccagtcccctgctcactccaaacccaggaattctgcccagttgatggggacacggtgggaaccagcttctgctgccttcacaaccaggcgccagtcctgtccatgggttatctcgcaaaccccagaggatctctgggaggaatgctactattaaccctatttcacaaacaaggaaatagaagagctcaaagaggttatgtaacttatctgtagccacgcagataatacaaagcagcaatctggacccattctgttcaaaacacttaacccttcgctatcatgccttggttcatctgggtctaatgtgctgagatcaagaaggtttaggacctaatggacagactcaagtcataacaatgctaagctctatttgtgtcccaagcactcctaagcattttatccctaactctacatcaaccccatgaaggagatactgttgatttccccatattagaagtagagagggaagctgaggcacacaaagactcatccacatgcccaagattcactgatagggaaaagtggaagcgagatttgaacccaggctgtttactcctaacctgtccaagccacctctcagacgacggtaggaatcagctggctgcttgtgagtacaggagttacagtccagtgggttatgttttttaagtctcaacatctaagcctggtcaggcatcagttcccctttttttgtgatttattttgtttttattttgttgttcattgtttaatttttccttttacaatgagaaggtcaccatcttgactcctaccttagccatttgttgaatcagactcatgacggctcctgggaagaagccagttcagatcataaaataaaacatatttattctttgtcatgggagtcattattttagaaactacaaactctccttgcttccatccttttttacatactcatgacacatgctcatcctgagtccttgaaaaggtatttttgaacatgtgtattaattataagcctctgaaaacctatggcccaaaccagaaatgatgttgattatataggtaaatgaaggatgctattgctgttctaattacctcattgtctcagtctcaaagtaggtcttcagctccctgtactttgggattttaatctaccaccacccataaatcaataaataattactttctttga

SEQ ID NO: 247 - protein, 287 aa, Homo sapiens
MSGSFDRKLSSILTDISSSLSCHAGSKDSPTLPESSVTDLGYYSAPQHDYYSGQPYGQTVNPYTYHHQFNLNGLAGTGAYSPKSEYTYGASYRQYGAYREQPLPAQDPVSVKEEPEAEVRMVNGKPKKVRKPRTIYSSYQLAALQRRFQKAQYLALPERAELAAQLGLTQTQVKIWFQNRRSKFKKLYKNGEVPLEHSPNNSDSMACNSPPSPALWDTSSHSTPAPARSQLPPPLPYSASPSYLDDPTNSWYHAQNLSGPHLQQQPPQPATLHHASPGPPPNPGAVY

SEQ ID NO: 248 - DNA, 2602 bp, Homo sapiens
  source 1..2602: mol_type = genomic DNA
agcatttgattgtggcttgggacgcgaggagaggcgcgcagcgaccgcctgacggcaggcaatggtgtaagcgcctctcggcctccccctccccccagacgcggccgggtcctcccttcgccttctggacacacacccctgcctcgtctcttccgcctctctcgcactccggtccgttcctgtcctctgcggaggccagccctggggaggtgcagcgcccgccaggatgagtggctccttcgatcgcaagctcagcagcatcctcaccgacatctccagctcccttagctgccatgcgggctccaaggactcgcctaccctgcccgagtcttctgtcactgacctgggctactacagcgctccccagcacgattactactcgggccagccctatggccagacggtgaacccctacacctaccaccaccaattcaatctcaatgggcttgcaggcacgggcgcttactcgcccaagtcggaatatacctacggagcctcctaccggcaatacggggcgtatcgggagcagccgctgccagcccaggacccagtgtcggtgaaggaggagccggaagcagaggtgcgcatggtgaatgggaagcccaagaaggtccgaaagccgcgtacgatctactccagctaccagctggccgccctgcagcgccgcttccagaaggcccagtacctggcgctgcccgagcgcgccgagctggccgcgcagctgggcctcacgcagacacaggtgaaaatctggttccagaaccgccgttccaagttcaagaaactctacaagaacggggaggtgccgctggagcacagtcccaataacagtgattccatggcctgcaactcaccaccatcacccgccctctgggacacctcttcccactccactccggcccctgcccgcagtcagctgcccccgccgctcccatacagtgcctcccccagctacctggacgaccccaccaactcctggtatcacgcacagaacctgagtggaccccacttacagcagcagccgcctcagccagccaccctgcaccatgcctctcccgggcccccgcccaaccctggggctgtgtactgagcacccatctggcctgcacccttgacaaaggaccccaggaccaggcagaaggcgcctccgtcctagccactcaggaatcatcgaggagcacagggaaaaggaactccctttccccctcccttgccccttcctccagggacccaagcgcttccagatgacaattgcatggaccaaggatgccccctgaacctccctccctctgcctagacactggggtacccctccagatgtggggacattccaccccagtggggacagccattcccctacctgctccaggagcctggattggctttaaatggctcatcatcttccagcttcttaaacttagtgcctgttcccagactggagaccttgggatgggggagagtgtggagggtttgcgggtcctgcctgtgctggggcacctggcaccgtggatcttaaaacttgccaggcctagttcctcctgagcctctggtggtctccccctgctcgagcggcccctcggccaataagacagtggacatcatgacgaggactccgggtggggacctgaactggtcaccgccctgcacttctagccctcatttaagatttgagggtgaaaccaaagaaaaccccctaagtgagggaatcttttaatatttgtggctttagaggaaagaactaaaggagccatctctctcccctctcctccgttccgagaggaggggtgggtctcagacgtttttcctatggacttatttcttccatgtccaggactttgcacaactttggttttaaaagctgttgaaaaataggaaaacaaagggcattgttcacagatagggccaagtctccccttgcaagggtgcctctgttctgtccctgcccccacctcaccttctctactcctccagtaagttggcagttttggtgccaaaccccaaatctccaaagagacataccaggcaagacaaacccccaaacacctcctttccggtggccttggaaacagattgctccgagctggagaatgtcgggtgaggtgtatgggagaggaggggagagttagaacttgtgcctttgggagtaaggggtaactgcctggagggctggtggcactgcccctccctgacccagacatcccaccaaagctaactttcccccacccctgatgcagtaaaacattgaaaaaaaaaaaaaaggagaggtagaagactgtagctatatatataaatatatagtaagttttttttttttaagagcaacagagagaagcagcctcctccctgctgcggtttcctatttatgtggccatgttcctcctggacggatctccctgtgtgtttcaagctgagagatgtgggctccggctggatttgggttttgtgggaggtgcaggggccaagagagacgtggtaggtctccaagagtcccacccgggggggaagaagcaaagccatctcccaccccctcccagccttctcatttctgctttcttactggactcatctttatatataatgttaataaaaaagacgaaaataa

SEQ ID NO: 249 - protein, 289 aa, Homo sapiens
MTGVFDRRVPSIRSGDFQAPFQTSAAMHHPSQESPTLPESSATDSDYYSPTGGAPHGYCSPTSASYGKALNPYQYQYHGVNGSAGSYPAKAYADYSYASSYHQYGGAYNRVPSATNQPEKEVTEPEVRMVNGKPKKVRKPRTIYSSFQLAALQRRFQKTQYLALPERAELAASLGLTQTQVKIWFQNKRSKIKKIMKNGEMPPEHSPSSSDPMACNSPQSPAVWEPQGSSRSLSHHPHAHPPTSNQSPASSYLENSASWYTSAASSINSHLPPPGSLQHPLALASGTLY

SEQ ID NO: 250 - DNA, 1418 bp, Homo sapiens
  source 1..1418: mol_type = genomic DNA
agcagtcagccggccggagacagagacttcacgactcccagtctcctcctcgccgcggccgccgcctcctccttctctcctcctcctcttcctcctcctccctcgctcccacagccatgtctgcttagaccagagcagccccacagccaactagggcagctgccgccgccacaacagcaaggacagccgctgccgccgcccgtgagcgatgacaggagtgtttgacagaagggtccccagcatccgatccggcgacttccaagctccgttccagacgtccgcagctatgcaccatccgtctcaggaatcgccaactttgcccgagtcttcagctaccgattctgactactacagccctacggggggagccccgcacggctactgctctcctacctcggcttcctatggcaaagctctcaacccctaccagtatcagtatcacggcgtgaacggctccgccgggagctacccagccaaagcttatgccgactatagctacgctagctcctaccaccagtacggcggcgcctacaaccgcgtcccaagcgccaccaaccagccagagaaagaagtgaccgagcccgaggtgagaatggtgaatggcaaaccaaagaaagttcgtaaacccaggactatttattccagctttcagctggccgcattacagagaaggtttcagaagactcagtacctcgccttgccggaacgcgccgagctggccgcctcgctgggattgacacaaacacaggtgaaaatctggtttcagaacaaaagatccaagatcaagaagatcatgaaaaacggggagatgcccccggagcacagtcccagctccagcgacccaatggcgtgtaactcgccgcagtctccagcggtgtgggagccccagggctcgtcccgctcgctcagccaccaccctcatgcccaccctccgacctccaaccagtccccagcgtccagctacctggagaactctgcatcctggtacacaagtgcagccagctcaatcaattcccacctgccgccgccgggctccttacagcacccgctggcgctggcctccgggacactctattagatgggctgctctctcttactctcttttttgggactactgtgttttgctgttctagaaaatcataaagaaaggaattcatatggggaagttcggaaaactgaaaaagattcatgtgtaaagcttttttttgcatgtaagttattgcatttcaaaagaccccccctttttttacagaggactttttttgcgcaactgtggacactttcaatggtgccttgaaatctatgacctcaacttttcaaaagacttttttcaatgttattttagccatgtaaataagtgtagatagaggaattaaactgtatattctggataaataaaattatttcgaccatgaaaa

SEQ ID NO: 251 - protein, 293 aa, Homo sapiens
MMTMTTMADGLEGQDSSKSAFMEFGQQQQQQQQQQQQQQQQQQQPPPPPPPPPQPHSQQSSPAMAGAHYPLHCLHSAAAAAAAGSHHHHHHQHHHHGSPYASGGGNSYNHRSLAAYPYMSHSQHSPYLQSYHNSSAAAQTRGDDTDQQKTTVIENGEIRFNGKGKKIRKPRTIYSSLQLQALNHRFQQTQYLALPERAELAASLGLTQTQVKIWFQNKRSKFKKLLKQGSNPHESDPLQGSAALSPRSPALPPVWDVSASAKGVSMPPNSYMPGYSHWYSSPHQDTMQRPQMM

SEQ ID NO: 252 - DNA, 2230 bp, Homo sapiens
  source 1..2230: mol_type = genomic DNA
attcgactcgtggcgtctgcatcaagtctgaaagcagacgcgcaactttcgcagaatccaccttaaaatctctgccttaaactgcaccagcccccaaaaaatccaaggggggaaagcaggcggggggagagcagattcccccctccccctctcctctcccatccctcctccttcctcctccctttgagttaacaaggccccgctcactatatctctttatattaaatatatatatatattagagaagagcgagggagagggagaaccacctccacccccctctttaaattcttttttttttttttttttttttttgcaaggatccaaagagctaaggtggctgcagaggggagagcggcgcgagccaagtgggggagggtggaggaaacccgggagaaggctttctccagcccccaaagtttttgatgatgaccatgactacgatggctgacggcttggaaggccaggactcgtccaaatccgccttcatggagttcgggcagcagcagcagcagcagcagcaacagcagcagcagcagcagcagcaacagcaacagccgccgccgccgccgccgccgccgccgcagccgcactcgcagcagagctccccggccatggcaggcgcgcactaccctctgcactgcctgcactcggcggcggcggcggcagcggccggctcgcaccaccaccaccaccaccagcaccaccaccacggctcgccctacgcgtcgggcggagggaactcctacaaccaccgctcgctcgccgcctacccctacatgagccactcgcagcacagcccttacctccagtcctaccacaacagcagcgcagccgcccagacgcgaggggacgacacagatcaacaaaaaactacagtgattgaaaacggggaaatcaggttcaatggaaaagggaaaaagattcggaagcctcggaccatttattccagcctgcagctccaggctttaaaccatcgctttcagcagacacagtatctggcccttccagagagagccgaactggcagcttccttaggactgacacaaacacaggtgaagatatggtttcagaacaaacgctctaagtttaagaaactgctgaagcagggcagtaatcctcatgagagcgaccccctccagggctcggcggccctgtcgccacgctcgccagcgctgcctccagtctgggacgtttctgcctcggccaagggtgtcagtatgccccccaacagctacatgcctggctattctcactggtactcctctccacaccaggacacgatgcagagaccacagatgatgtgagttgcccaagggaacaccctagggaaacgtctgaacaaggaaaagaggatccgggacctgcttgtatctgcgaaaaggagccaaaggagcaggcttaggagagctcataagtgtggcaagaagccgactaggctcattctctctccctctctctctctctccctctcctttctttttacttcttcctttcctccattccttctttctttccttttcctttctacctttcttttctttttgcctttcaccttttttctcatttaccttctctcttgagcaacgtcagtaattgatcttgcatctcagagagagagaaagagcatgtgtgagagagaaactggtttctatgccagcactcctgaaaccccttactgtaaggatattttctcttaccccttgggatccaggctctgagtctcttctctttgggagtatccatcaaaatgactttttttaaaaacagattttcccccaaccagaagaatctgcacaaacttggcagcgtttttacttgtttaatgagtttaagacattacatggtgaaagagaagcattttggactcctgcatttttatttaccattcccagactgacgagaaaaagaaaattcctcacataacagcccttctctaaagaaaaaggaaaaagtggctttgattaaaaaaaaacaaaacaaaaaccactctttccccaccccacccccccaaaccctgaactggaatcaggaaagacggaggaaacaatcaaaatcaccattctattgctttgacacctttactaggtgaattggtggcattcacaaagctaatagggacgtttatatcaagaaacatttctgtatatattgttgaattttagttgtacatatactttgtatgtttttgtcttctttcatatatggagtaaaagccacaaaacgctga

SEQ ID NO: 253 - protein, 73 aa, Homo sapiens
MSAETASGPTEDQVEILEYNFNKVDKHPDSTTLCLIAAEAGLSEEETQKWFKQRLAKWRRSEGLPSECRSVTD

SEQ ID NO: 254 - DNA, 1198 bp, Homo sapiens
  source 1..1198: mol_type = genomic DNA
gagacagaaggccgcctaccggggaggccggaggccggctagtcgcggactcgggcgaacccaccctcgcgatctgtcaagtctgtccccaggggaggtccccctttcgggaggaagtttttaaggggatttctcaaaatcacccccgcgcttccttcactccttccttagagccggaggtcggtgagggcccgcggaatcatctatctcgcccccgtcgcagcgcgcagggaccatgtcggcggagaccgcgagcggccccacagaggaccaggtggaaatcctggagtacaacttcaacaaggtcgacaagcacccggattccaccacgctgtgcctcatcgcggccgaggcaggcctttccgaggaggagacccagaaatggtttaagcagcgcctggcaaagtggcggcgctcagaaggcctgccctcagagtgcagatccgtcacagactaaggagatggcaggcattgacagcttcactccatgaaggccatctctgtttctctcctccgcttaaccaagctgttgtggtttttcagcatagtgttgtatgttccattgctagctgtcctgctgtttaacacagtgttgtattttttttctaaatgtacataattagaaaagaaaataacaataggaagctatgtgtatcttctgtgtaaagcagtggcttcactggaaaaatggtgtggctagcatttccctttgagtcatgatgacagatggtgtgaaaaccatctaagtttgcttttgaccatcacctcccagtagcaatttgctttcataatccatttagcaatccaggcctctgttgaaaagataatatgagggagaagggaacacatttccttctgaacttacttccctaagtcactttccttatgtatcatctaatacaatgatggttgagtgaaaatacagaaggggtgtttgagtattcagatttcataaaacacttccttggaatatagctgcattaacttggaaagaagcctgttgggccagaagacagaaactccaactggcaaaaaagcaagcatctaagaaaaaaaaccaccaaagttcttgaatttactatatttaaatgcattggttaagtttattttgctaaataaagtgaactgctttttgtctctaaaatgatattctaaataaaaccttaactttttgttgaagatgcactgaaaaaaaaaa

SEQ ID NO: 255 - protein, 239 aa, Homo sapiens
MPGGAHGPPPGYGCAAAGYLDGRLEPLYERVGAPALRPLVIKQEPREEDEAKQLALAGLFPYQPPPPPPPSHPHPHPPPAHLAAPHLQFQIAHCGQTTMHLQPGHPTPPPTPVPSPHPAPALGAAGLPGPGSALKGLGAAHPDLRASGGSGAGKAKKSVDKNSNEYRVRRERNNIAVRKSRDKAKQRNVETQQKVLELTSDNDRLRKRVEQLSRELDTLRGIFRQLPESSLVKAMGNCA

SEQ ID NO: 256 - DNA, 2631 bp, Homo sapiens
  source 1..2631: mol_type = genomic DNA
tataaaagctgggccggcgcgggccgggccattcgcgacccggaggtgcgcgggcgcgggcgagcagggtctccgggtgggcggcggcgacgccccgcgcaggctggaggccgccgaggctcgccatgccgggagaactctaactcccccatggagtcggccgacttctacgaggcggagccgcggcccccgatgagcagccacctgcagagccccccgcacgcgcccagcagcgccgccttcggctttccccggggcgcgggccccgcgcagcctcccgccccacctgccgccccggagccgctgggcggcatctgcgagcacgagacgtccatcgacatcagcgcctacatcgacccggccgccttcaacgacgagttcctggccgacctgttccagcacagccggcagcaggagaaggccaaggcggccgtgggccccacgggcggcggcggcggcggcgactttgactacccgggcgcgcccgcgggccccggcggcgccgtcatgcccgggggagcgcacgggcccccgcccggctacggctgcgcggccgccggctacctggacggcaggctggagcccctgtacgagcgcgtcggggcgccggcgctgcggccgctggtgatcaagcaggagccccgcgaggaggatgaagccaagcagctggcgctggccggcctcttcccttaccagccgccgccgccgccgccgccctcgcacccgcacccgcacccgccgcccgcgcacctggccgccccgcacctgcagttccagatcgcgcactgcggccagaccaccatgcacctgcagcccggtcaccccacgccgccgcccacgcccgtgcccagcccgcaccccgcgcccgcgctcggtgccgccggcctgccgggccctggcagcgcgctcaaggggctgggcgccgcgcaccccgacctccgcgcgagtggcggcagcggcgcgggcaaggccaagaagtcggtggacaagaacagcaacgagtaccgggtgcggcgcgagcgcaacaacatcgcggtgcgcaagagccgcgacaaggccaagcagcgcaacgtggagacgcagcagaaggtgctggagctgaccagtgacaatgaccgcctgcgcaagcgggtggaacagctgagccgcgaactggacacgctgcggggcatcttccgccagctgccagagagctccttggtcaaggccatgggcaactgcgcgtgaggcgcgcggctgtgggaccgccctgggccagcctccggcggggacccagggagtggtttggggtcgccggatctcgaggcttgcccgagccgtgcgagccaggactaggagattccggtgcctcctgaaagcctggcctgctccgcgtgtcccctcccttcctctgcgccggacttggtgcgtctaagatgagggggccaggcggtggcttctccctgcgaggaggggagaattcttggggctgagctgggagcccggcaactctagtatttaggataaccttgtgccttggaaatgcaaactcaccgctccaatgcctactgagtagggggagcaaatcgtgccttgtcattttatttggaggtttcctgcctccttcccgaggctacagcagacccccatgagagaaggaggggagcaggcccgtggcaggaggagggctcagggagctgagatcccgacaagcccgccagccccagccgctcctccacgcctgtccttagaaaggggtggaaacatagggacttggggcttggaacctaaggttgttcccctagttctacatgaaggtggagggtctctagttccacgcctctcccacctccctccgcacacaccccaccccagcctgctataggctgggcttccccttggggcggaactcactgcgatgggggtcaccaggtgaccagtgggagcccccaccccgagtcacaccagaaagctaggtcgtgggtcagctctgaggatgtatacccctggtgggagagggagacctagagatctggctgtggggcgggcatggggggtgaagggccactgggaccctcagccttgtttgtactgtatgccttcagcattgcctaggaacacgaagcacgatcagtccatcccagagggaccggagttatgacaagctttccaaatattttgctttatcagccgatatcaacacttgtatctggcctctgtgccccagcagtgccttgtgcaatgtgaatgtgcgcgtctctgctaaaccaccattttatttggtttttgttttgttttggttttgctcggatacttgccaaaatgagactctccgtcggcagctgggggaagggtctgagactccctttccttttggttttgggattacttttgatcctgggggaccaatgaggtgaggggggttctcctttgccctcagctttccccagcccctccggcctgggctgcccacaaggcttgtcccccagaggccctggctcctggtcgggaagggaggtggcctcccgccaacgcatcactggggctgggagcagggaaggacggcttggttctcttcttttggggagaacgtagagtctcactctagatgttttatgtattatatctataatataaacatatcaaagtcaa

SEQ ID NO: 257 - protein, 351 aa, Homo sapiens
MTEMSFLSSEVLVGDLMSPFDQSGLGAEESLGLLDDYLEVAKHFKPHGFSSDKAKAGSSEWLAVDGLVSPSNNSKEDAFSGTDWMLEKMDLKEFDLDALLGIDDLETMPDDLLTTLDDTCDLFAPLVQETNKQPPQTVNPIGHLPESLTKPDQVAPFTFLQPLPLSPGVLSSTPDHSFSLELGSEVDITEGDRKPDYTAYVAMIPQCIKEEDTPSDNDSGICMSPESYLGSPQHSPSTRGSPNRSLPSPGVLCGSARPKPYDPPGEKMVAAKVKGEKLDKKLKKMEQNKTAATRYRQKKRAEQEALTGECKELEKKNEALKERADSLAKEIQYLKDLIEEVRKARGKKRVP

SEQ ID NO: 258 - DNA, 2041 bp, Homo sapiens
  source 1..2041: mol_type = genomic DNA
agccatttctactttgcccgcccacagatgtagttttctctgcgcgtgtgcgttttccctcctccccgccctcagggtccacggccaccatggcgtattaggggcagcagtgcctgcggcagcattggcctttgcagcggcggcagcagcaccaggctctgcagcggcaacccccagcggcttaagccatggcgtgagtaccggggcgggtcgtccagctgtgctcctggggccggcgcgggttttggattggtggggtgcggcctggggccagggcggtgccgccaagggggaagcgatttaacgagcgcccgggacgcgtggtctttgcttgggtgtccccgagacgctcgcgtgcctgggatcgggaaagcgtagtcgggtgcccggactgcttccccaggagccctacagccctcggaccccgagccccgcaagggtcccaggggtcttggctgttgccccacgaaacgtggcaggaaccaagatggcggcggcagggcggcggcgcgggcgtgagtcaagggcgggcggtgggcggggcgcggccgccctggccgtatttggacgtggggacggagcgctttcctcttggcggccggtggaagaatcccctggtctccgtgagcgtccattttgtggaacctgagttgcaagcagggaggggcaaatacaactgccctgttcccgattctctagatggccgatctagagaagtcccgcctcataagtggaaggatgaaattctcagaacagctaacctctaatgggagttggcttctgattctcattcaggcttctcacggcattcagcagcagcgttgctgtaaccgacaaagacaccttcgaattaagcacattcctcgattccagcaaagcaccgcaacatgaccgaaatgagcttcctgagcagcgaggtgttggtgggggacttgatgtcccccttcgaccagtcgggtttgggggctgaagaaagcctaggtctcttagatgattacctggaggtggccaagcacttcaaacctcatgggttctccagcgacaaggctaaggcgggctcctccgaatggctggctgtggatgggttggtcagtccctccaacaacagcaaggaggatgccttctccgggacagattggatgttggagaaaatggatttgaaggagttcgacttggatgccctgttgggtatagatgacctggaaaccatgccagatgaccttctgaccacgttggatgacacttgtgatctctttgcccccctagtccaggagactaataagcagcccccccagacggtgaacccaattggccatctcccagaaagtttaacaaaacccgaccaggttgcccccttcaccttcttacaacctcttcccctttccccaggggtcctgtcctccactccagatcattcctttagtttagagctgggcagtgaagtggatatcactgaaggagataggaagccagactacactgcttacgttgccatgatccctcagtgcataaaggaggaagacaccccttcagataatgatagtggcatctgtatgagcccagagtcctatctggggtctcctcagcacagcccctctaccaggggctctccaaataggagcctcccatctccaggtgttctctgtgggtctgcccgtcccaaaccttacgatcctcctggagagaagatggtagcagcaaaagtaaagggtgagaaactggataagaagctgaaaaaaatggagcaaaacaagacagcagccactaggtaccgccagaagaagagggcggagcaggaggctcttactggtgagtgcaaagagctggaaaagaagaacgaggctctaaaagagagggcggattccctggccaaggagatccagtacctgaaagatttgatagaagaggtccgcaaggcaagggggaagaaaagggtcccctagttgaggatagtcaggagcgtcaatgtgcttgtacatagagtgctgtagctgtgtgttccaataaattattttgtagggaaagtaaaaaaaaaaaaaaa

SEQ ID NO: 259 - protein, 465 aa, Homo sapiens
MNVTSLFSFTSPAVKRLLGWKQGDEEEKWAEKAVDALVKKLKKKKGAMEELEKALSCPGQPSNCVTIPRSLDGRLQVSHRKGLPHVIYCRVWRWPDLQSHHELKPLECCEFPFGSKQKEVCINPYHYKRVESPVLPPVLVPRHSEYNPQHSLLAQFRNLGQNEPHMPLNATFPDSFQQPNSHPFPHSPNSSYPNSPGSSSSTYPHSPTSSDPGSPFQMPADTPPPAYLPPEDPMTQDGSQPMDTNMMAPPLPSEINRGDVQAVAYEEPKHWCSIVYYELNNRVGEAFHASSTSVLVDGFTDPSNNKNRFCLGLLSNVNRNSTIENTRRHIGKGVHLYYVGGEVYAECLSDSSIFVQSRNCNYHHGFHPTTVCKIPSGCSLKIFNNQEFAQLLAQSVNHGFETVYELTKMCTIRMSFVKGWGAEYHRQDVTSTPCWIEIHLHGPLQWLDKVLTQMGSPHNPISSVS

SEQ ID NO: 260 - DNA, 2879 bp, Homo sapiens
  source 1..2879: mol_type = genomic DNA
cactgcatgtgtattcgtgagttcgcggttgaacaactgttcctttactctgctccctgtctttgtgctgactgggttacttttttaaacactaggaatggtaatttctactcttctggacttcaaactaagaagttaaagagacttctctgtaaataaacaaatctcttctgctgtccttttgcatttggagacagctttatttcaccatatccaaggagtataactagtgctgtcattatgaatgtgacaagtttattttcctttacaagtccagctgtgaagagacttcttgggtggaaacagggcgatgaagaagaaaaatgggcagagaaagctgttgatgctttggtgaaaaaactgaagaaaaagaaaggtgccatggaggaactggaaaaggccttgagctgcccagggcaaccgagtaactgtgtcaccattccccgctctctggatggcaggctgcaagtctcccaccggaagggactgcctcatgtcatttactgccgtgtgtggcgctggcccgatcttcagagccaccatgaactaaaaccactggaatgctgtgagtttccttttggttccaagcagaaggaggtctgcatcaatccctaccactataagagagtagaaagccctgtacttcctcctgtgctggttccaagacacagcgaatataatcctcagcacagcctcttagctcagttccgtaacttaggacaaaatgagcctcacatgccactcaacgccacttttccagattctttccagcaacccaacagccacccgtttcctcactctcccaatagcagttacccaaactctcctgggagcagcagcagcacctaccctcactctcccaccagctcagacccaggaagccctttccagatgccagctgatacgcccccacctgcttacctgcctcctgaagaccccatgacccaggatggctctcagccgatggacacaaacatgatggcgcctcccctgccctcagaaatcaacagaggagatgttcaggcggttgcttatgaggaaccaaaacactggtgctctattgtctactatgagctcaacaatcgtgtgggtgaagcgttccatgcctcctccacaagtgtgttggtggatggtttcactgatccttccaacaataagaaccgtttctgccttgggctgctctccaatgttaaccggaattccactattgaaaacaccaggcggcatattggaaaaggagttcatctttattatgttggaggggaggtgtatgccgaatgccttagtgacagtagcatctttgtgcaaagtcggaactgcaactaccatcatggatttcatcctactactgtttgcaagatccctagtgggtgtagtctgaaaatttttaacaaccaagaatttgctcagttattggcacagtctgtgaaccatggatttgagacagtctatgagcttacaaaaatgtgtactatacgtatgagctttgtgaagggctggggagcagaataccaccgccaggatgttactagcaccccctgctggattgagatacatctgcacggccccctccagtggctggataaagttcttactcaaatgggttcacctcataatcctatttcatctgtatcttaaatggccccaggcatctgcctctggaaaactattgagccttgcatgtacttgaaggatggatgagtcagacacgattgagaactgacaaaggagccttgataatacttgacctctgtgaccaactgttggattcagaaatttaaacaaaaaaaaaaaaaaacacacacaccttggtaacatactgttgatatcaagaacctgtttagtttacattgtaacattctattgtaaaatcaactaaaattcagacttttagcaggactttgtgtacagttaaaggagagatggccaagccagggacaaattgtctattagaaaacggtcctaagagattctttggtgtttggcactttaaggtcatcgttgggcagaagtttagcattaatagttgttctgaaacgtgttttatcaggtttagagcccatgttgagtcttcttttcatgggttttcataatattttaaaactatttgtttagcgatggttttgttcgtttaagtaaaggttaatcttgatgatatacataataatctttctaaaattgtatgctgaccatacttgctgtcagaataatgctaggcatatgctttttgctaaatatgtatgtacagagtatttggaagttaagaattgattagactagtgaatttaggagtatttgaggtgggtggggggaagagggaaatgacaactgcaaatgtagactatactgtaaaaattcagtttgttgctttaaagaaacaaactgatacctgaattttgctgtgtttccattttttagagatttttatcatttttttctctctcggcattcttttttctcatactcttcaaaaagcagttctgcagctggttaattcatgtaactgtgagagcaaatgaataattcctgctattctgaaattgcctacatgtttcaataccagttatatggagtgcttgaatttaataagcagtttttacggagtttacagtacagaaataggctttaattttcaagtgaattttttgccaaacttagtaactctgttaaatatttggaggatttaaagaacatcccagtttgaattcatttcaaactttttaaatttttttgtactatgtttggttttattttccttctgttaatcttttgtattcacttatgctctcgtacattgagtacttttattccaaaactagtgggttttctctactggaaattttcaataaacctgtcattattgcttactttgattaaaa

SEQ ID NO: 261 - protein, 615 aa, Homo sapiens
MGLKAAQKTLFPLRSIDDVVRLFAAELGREEPDLVLLSLVLGFVEHFLAVNRVIPTNVPELTFQPSPAPDPPGGLTYFPVADLSIIAALYARFTAQIRGAVDLSLYPREGGVSSRELVKKVSDVIWNSLSRSYFKDRAHIQSLFSFITGWSPVGTKLDSSGVAFAVVGACQALGLRDVHLALSEDHAWVVFGPNGEQTAEVTWHGKGNEDRRGQTVNAGVAERSWLYLKGSYMRCDRKMEVAFMVCAINPSIDLHTDSLELLQLQQKLLWLLYDLGHLERYPMALGNLADLEELEPTPGRPDPLTLYHKGIASAKTYYRDEHIYPYMYLAGYHCRNRNVREALQAWADTATVIQDYNYCREDEEIYKEFFEVANDVIPNLLKEAASLLEAGEERPGEQSQGTQSQGSALQDPECFAHLLRFYDGICKWEEGSPTPVLHVGWATFLVQSLGRFEGQVRQKVRIVSREAEAAEAEEPWGEEAREGRRRGPRRESKPEEPPPPKKPALDKGLGTGQGAVSGPPRKPPGTVAGTARGPEGGSTAQVPAPAASPPPEGPVLTFQSEKMKGMKELLVATKINSSAIKLQLTAQSQVQMKKQKVSTPSDYTLSFLKRQRKGL

SEQ ID NO: 262 - DNA, 2785 bp, Homo sapiens
  source 1..2785: mol_type = genomic DNA
ggtgtccggagccgcggacctagagatcccagaagccacagcgcagcggcccggcccgccactatttccaggctcagcggggcaggggccgccgcccaccgcccgccgccatggggctgaaggccgcccagaagacgctgttcccgctgcgctccatcgacgacgtggtgcgcctgtttgctgccgagctgggccgagaggagccggacctggtgctcctttccttggtgctgggcttcgtggagcattttctggctgtcaaccgcgtcatccctaccaacgttcccgagctcaccttccagcccagccccgcccccgacccgcctggcggcctcacctactttcccgtggccgacctgtctatcatcgccgccctctatgcccgcttcaccgcccagatccgaggcgccgtcgacctgtccctctatcctcgagaagggggtgtctccagccgtgagctggtgaagaaggtctccgatgtcatatggaacagcctcagccgctcctacttcaaggatcgggcccacatccagtccctcttcagcttcatcacaggttggagcccagtaggcaccaaattggacagctccggtgtggcctttgctgtggttggggcctgccaggccctgggtctccgggatgtccacctcgccctgtctgaggatcatgcctgggtagtgtttgggcccaatggggagcagacagctgaggtcacctggcacggcaagggcaacgaggaccgcaggggccagacagtcaatgccggtgtggctgagcggagctggctgtacctgaaaggatcatacatgcgctgtgaccgcaagatggaggtggcgttcatggtgtgtgccatcaacccttccattgacctgcacaccgactcgctggagcttctgcagctgcagcagaagctgctctggctgctctatgacctgggacatctggaaaggtaccccatggccttagggaacctggcagatctagaggagctggagcccacccctggccggccagacccactcaccctctaccacaagggcattgcctcagccaagacctactatcgggatgaacacatctacccctacatgtacctggctggctaccactgtcgcaaccgcaatgtgcgggaagccctgcaggcctgggcggacacggccactgtcatccaggactacaactactgccgggaagacgaggagatctacaaggagttctttgaagtagccaatgatgtcatccccaacctgctgaaggaggcagccagcttgctggaggcgggcgaggagcggccgggggagcaaagccagggcacccagagccaaggttccgccctccaggaccctgagtgcttcgcccacctgctgcgattctacgacggcatctgcaaatgggaggagggcagtcccacgcctgtgctgcacgtgggctgggccacctttcttgtgcagtccctaggccgttttgagggacaggtgcggcagaaggtgcgcatagtgagccgagaggccgaggcggccgaggccgaggagccgtggggcgaggaagcccgggaaggccggcggcggggcccacggcgggagtccaagccagaggagcccccgccgcccaagaagccagcactggacaagggcctgggcaccggccagggtgcagtgtcaggacccccccggaagcctcctgggactgtcgctggcacagcccgaggccctgaaggtggcagcacggctcaggtgccagcacccgcagcatcaccaccgccggagggtccagtgctcactttccagagtgagaagatgaagggcatgaaggagctgctggtggccaccaagatcaactcgagcgccatcaagctgcaactcacggcacagtcgcaagtgcagatgaagaagcagaaagtgtccacccctagtgactacactctgtctttcctcaagcggcagcgcaaaggcctctgaactactggggacttcggaccgcttgtggggacccaggctccgcccttagtcccccaactctgagcccatgttctgcccccagcccaaaggggacaggcctcacctctacccaaaccctaggttcccggtcccgagtacagtctgtatcaaacccacgattttctccagctcagaacccagggctctgccccagtcgttagaatataggtctcttctcccagaatcccagccggccaatggaaacctcacgctgggtcctaattaccagtctttaaaggcccagcccctagaaacccaagctcctcctcggaaccgctcacctagagccagaccaacgttactcagggctcctcccagcttgtaggagctgaggtttcacccttaacccaaggagcacaggtcccacctccagcccgggagcctaggaccactcagcccctaggagtatatttccgcacttcagaattccatatcttgcgaatccaagctccctgccccaaataacttcagtcctgctccagaatttggaaatcctagtttcctctccttcgtatcccgagtctgggacacaaaactccgcccccagcctatgagcatcctgagccccgccctcttcctgacgaaactggccccggatcagagcaggacctcccttccgaccctctgggaacctcccagaggtccagcccatctcggagcatcccggaggaaatctgcagagggttaggagtgggtgacaagagcctgatctcttcctgttttgtacatagatttatttttcagttccaagaaagatgaatacattttgttaaaaaaaataaaaaaaa

SEQ ID NO: 263 - protein, 303 aa, Homo sapiens
MAPAADMTSLPLGVKVEDSAFGKPAGGGAGQAPSAAAATAAAMGADEEGAKPKVSPSLLPFSVEALMADHRKPGAKESALAPSEGVQAAGGSAQPLGVPPGSLGAPDAPSSPRPLGHFSVGGLLKLPEDALVKAESPEKPERTPWMQSPRFSPPPARRLSPPACTLRKHKTNRKPRTPFTTAQLLALERKFRQKQYLSIAERAEFSSSLSLTETQVKIWFQNRRAKAKRLQEAELEKLKMAAKPMLPPAAFGLSFPLGGPAAVAAAAGASLYGASGPFQRAALPVAPVGLYTAHVGYSMYHLT

SEQ ID NO: 264 - DNA, 1940 bp, Homo sapiens
  source 1..1940: mol_type = genomic DNA
agggcccggagccggcgagtgctcccgggaactctgcctgcgcggcggcagcgaccggaggccaggcccagcacgccggagctggcctgctggggaggggcgggaggcgcgcgcgggagggtccgcccggccagggccccgggcgctcgcagaggccggccgcgctcccagcccgcccggagcccatgcccggcggctggccagtgctgcggcagaagggggggcccggctctgcatggccccggctgctgacatgacttctttgccactcggtgtcaaagtggaggactccgccttcggcaagccggcggggggaggcgcgggccaggcccccagcgccgccgcggccacggcagccgccatgggcgcggacgaggagggggccaagcccaaagtgtccccttcgctcctgcccttcagcgtggaggcgctcatggccgaccacaggaagccgggggccaaggagagcgccctggcgccctccgagggcgtgcaggcggcgggtggctcggcgcagccactgggcgtcccgccggggtcgctgggagccccggacgcgccctcttcgccgcggccgctcggccatttctcggtggggggactcctcaagctgccagaagatgcgctcgtcaaagccgagagccccgagaagcccgagaggaccccgtggatgcagagcccccgcttctccccgccgccggccaggcggctgagccccccagcctgcaccctccgcaaacacaagacgaaccgtaagccgcggacgcccttcaccaccgcgcagctgctggcgctggagcgcaagttccgccagaagcagtacctgtccatcgccgagcgcgcggagttctccagctcgctcagcctcactgagacgcaggtgaagatatggttccagaaccgccgcgccaaggcaaagagactacaagaggcagagctggagaagctgaagatggccgccaagcccatgctgccaccggctgccttcggcctctccttccctctcggcggccccgcagctgtagcggccgcggcgggtgcctcgctctacggtgcctctggccccttccagcgcgccgcgctgcctgtggcgcccgtgggactctacacggcccatgtgggctacagcatgtaccacctgacatagagggtcccaggtcgcccacctgtgggccagccgattcctccagccctggtgctgtacccccgacgtgctcccctgctcggcaccgccagccgccttccctttaaccctcacactgctccagtttcacctctttgctccctgagttcactctccgaagtctgatccctgccaaaaagtggctggaagagtcccttagtactcttctagcatttagatctacactctcgagttaaagatggggaaactgagggcagagaggttaacagatttatctaaggtccccagcagaattgacagttgaacagagctagaggccatgtctcctgcatagcttttccctgtcctgacaccaggcaagaaaagcgcagagaaatcggtgtctgacgattttggaaatgagaacaatctcaaaaaaaaaaaaaaaaaaaaaaaaaaaaaaaaaaaagaaaagagaaaaaaaagactagccagccaggaagatgaatcctagcttcttccattggaaaatttaagacaagttcaacaacaaaacatttgctctggggggcagggaaaacacagatgtgttgcaaaggtaggttgaagggacctctctcttaccagtaccagaaacacaattgtaaaattaaaaaaaaaaaaaaactctttctatttaacagtacatttgtgtggctctcaaacatccctttggaagggattgtgtgtactatgtaatatactgtatatttgaaattttattatcatttatattatagctatatttgttaaataaattaattttaagctacaaaaa

SEQ ID NO: 265 - protein, 134 aa, Homo sapiens
MASPSKGNDLFSPDEEGPAVVAGPGPGPGGAEGAAEERRVKVSSLPFSVEALMSDKKPPKEASPLPAESASAGATLRPLLLSGHGAREAHSPGPLVKPFETASVKSENSEDGAAWMQEPGRYSPPPSECAPGQE

SEQ ID NO: 266 - DNA, 2224 bp, Homo sapiens
  source 1..2224: mol_type = genomic DNA
gcagcaaaaaagtttgagtcgccgctgccgggttgccagcggagtcgcgcgtcgggagctacgtagggcagagaagtcatggcttctccgtccaaaggcaatgacttgttttcgcccgacgaggagggcccagcagtggtggccggaccaggcccggggcctgggggcgccgagggggccgcggaggagcgccgcgtcaaggtctccagcctgcccttcagcgtggaggcgctcatgtccgacaagaagccgcccaaggaggcgtccccgctgccggccgaaagcgcctcggccggggccaccctgcggccactgctgctgtcggggcacggcgctcgggaagcgcacagccccgggccgctggtgaagcccttcgagaccgcctcggtcaagtcggaaaattcagaagatggagcggcgtggatgcaggaacccggccgatattcgccgccgccaagtgagtgcgcgccggggcaggagtaggaggacatatgagccctaccacctgcaccctgaggaaacacaagaccaatcggaagccgcgcacgccctttaccacatcccagctcctcgccctggagcgcaagttccgtcagaaacagtacctctccattgcagagcgtgcagagttctccagctctctgaacctcacagagacccaggtcaaaatctggttccagaaccgaagggccaaggcgaaaagactgcaggaggcagaactggaaaagctgaaaatggctgcaaaacctatgctgccctccagcttcagtctccctttccccatcagctcgcccctgcaggcagcgtccatatatggagcatcctacccgttccatagacctgtgcttcccatcccgcctgtgggactctatgccacgccagtgggatatggcatgtaccacctgtcctaaggaagaccagatcaatagactccatgatggatgcttgtttcaaagggtttcctctccctctccacgaaggcagtaccagccagtactcctgctctgctaaccctgcgtgcaccaccctaagcggctaggctgacagggccacacgacatagctgaaatttgttctgtaggcggaggcaccaagccctgttttcttggtgtaatcttccagatgcccccttttcctttcacaaagattggctctgatggtttttatgtataaatatatatatataataaaatataatacatttttatacagcagacgtaaaaattcaaattattttaaaaggcaaaatttatatacatatgtgctttttttctatatctcaccttcccaaaagacactgtgtaagtccatttgttgtattttcttaaagagggagacaaattatttgcaaaatgtgctaaagtcaatgatttttacgggattattgacttctgcttatggaaaacaaagaaacagacacaatgcacacagaaaatattagatatggagagattattcaaagtgaaggggacacatcatatttctgcattttacttgcattaaaagaaacctctttatatactacagttgttcctatctctcccccgccccccaccgccccaccacacacatatttttaaagtttttccttttttaagaatatttttgtaagaccaatacctgggatgagaagaatcctgagactgcctggaggtgaggtagaaaattagaaatacttcctaattcttctcaaggctgttggtaactttatttcagataattggagagtaaaatgttaaaacctgttgagaggaattgatggtttctgagaaatactaggtacattcatcctcacagattgcaaaggtgatttgggtgggggtttagtaattttctgcttaaaaaatgagtatcttgtaaccattacctatatgctaaatattcttgaacaattagtagatccagaaagaaaaaaaaatatgctttctctgtgtgtgtacctgttgtatgtcctaaacttattagaaaattttatatacttttttacatgttggggggcagaaggtaaagccatgttttgacttggtgaaaatgggattgtcaaacagcccattaagttccctggtatttcaccttcctgtccatctgtcccctccctccggtatacctttatccctttgaaagggtgcttgtacaatttgatatattttattgaagagttatctcttattctgaattaaattaagcatttgttttattgcagtaaagtttgtccaaactcacaa

SEQ ID NO: 267 - protein, 2818 aa, Homo sapiens
MAAHRPVEWVQAVVSRFDEQLPIKTGQQNTHTKVSTEHNKECLINISKYKFSLVISGLTTILKNVNNMRIFGEAAEKNLYLSQLIILDTLEKCLAGQPKDTMRLDETMLVKQLLPEICHFLHTCREGNQHAAELRNSASGVLFSLSCNNFNAVFSRISTRLQELTVCSEDNVDVHDIELLQYINVDCAKLKRLLKETAFKFKALKKVAQLAVINSLEKAFWNWVENYPDEFTKLYQIPQTDMAECAEKLFDLVDGFAESTKRKAAVWPLQIILLILCPEIIQDISKDVVDENNMNKKLFLDSLRKALAGHGGSRQLTESAAIACVKLCKASTYINWEDNSVIFLLVQSMVVDLKNLLFNPSKPFSRGSQPADVDLMIDCLVSCFRISPHNNQHFKICLAQNSPSTFHYVLVNSLHRIITNSALDWWPKIDAVYCHSVELRNMFGETLHKAVQGCGAHPAIRMAPSLTFKEKVTSLKFKEKPTDLETRSYKYLLLSMVKLIHADPKLLLCNPRKQGPETQGSTAELITGLVQLVPQSHMPEIAQEAMEALLVLHQLDSIDLWNPDAPVETFWEISSQMLFYICKKLTSHQMLSSTEILKWLREILICRNKFLLKNKQADRSSCHFLLFYGVGCDIPSSGNTSQMSMDHEELLRTPGASLRKGKGNSSMDSAAGCSGTPPICRQAQTKLEVALYMFLWNPDTEAVLVAMSCFRHLCEEADIRCGVDEVSVHNLLPNYNTFMEFASVSNMMSTGRAALQKRVMALLRRIEHPTAGNTEAWEDTHAKWEQATKLILNYPKAKMEDGQAAESLHKTIVKRRMSHVSGGGSIDLSDTDSLQEWINMTGFLCALGGVCLQQRSNSGLATYSPPMGPVSERKGSMISVMSSEGNADTPVSKFMDRLLSLMVCNHEKVGLQIRTNVKDLVGLELSPALYPMLFNKLKNTISKFFDSQGQVLLTDTNTQFVEQTIAIMKNLLDNHTEGSSEHLGQASIETMMLNLVRYVRVLGNMVHAIQIKTKLCQLVEVMMARRDDLSFCQEMKFRNKMVEYLTDWVMGTSNQAADDDVKCLTRDLDQASMEAVVSLLAGLPLQPEEGDGVELMEAKSQLFLKYFTLFMNLLNDCSEVEDESAQTGGRKRGMSRRLASLRHCTVLAMSNLLNANVDSGLMHSIGLGYHKDLQTRATFMEVLTKILQQGTEFDTLAETVLADRFERLVELVTMMGDQGELPIAMALANVVPCSQWDELARVLVTLFDSRHLLYQLLWNMFSKEVELADSMQTLFRGNSLASKIMTFCFKVYGATYLQKLLDPLLRIVITSSDWQHVSFEVDPTRLEPSESLEENQRNLLQMTEKFFHAIISSSSEFPPQLRSVCHCLYQVVSQRFPQNSIGAVGSAMFLRFINPAIVSPYEAGILDKKPPPRIERGLKLMSKILQSIANHVLFTKEEHMRPFNDFVKSNFDAARRFFLDIASDCPTSDAVNHSLSFISDGNVLALHRLLWNNQEKIGQYLSSNRDHKAVGRRPFDKMATLLAYLGPPEHKPVADTHWSSLNLTSSKFEEFMTRHQVHEKEEFKALKTLSIFYQAGTSKAGNPIFYYVARRFKTGQINGDLLIYHVLLTLKPYYAKPYEIVVDLTHTGPSNRFKTDFLSKWFVVFPGFAYDNVSAVYIYNCNSWVREYTKYHERLLTGLKGSKRLVFIDCPGKLAEHIEHEQQKLPAATLALEEDLKVFHNALKLAHKDTKVSIKVGSTAVQVTSAERTKVLGQSVFLNDIYYASEIEEICLVDENQFTLTIANQGTPLTFMHQECEAIVQSIIHIRTRWELSQPDSIPQHTKIRPKDVPGTLLNIALLNLGSSDPSLRSAAYNLLCALTCTFNLKIEGQLLETSGLCIPANNTLFIVSISKTLAANEPHLTLEFLEECISGFSKSSIELKHLCLEYMTPWLSNLVRFCKHNDDAKRQRVTAILDKLITMTINEKQMYPSIQAKIWGSLGQITDLLDVVLDSFIKTSATGGLGSIKAEVMADTAVALASGNVKLVSSKVIGRMCKIIDKTCLSPTPTLEQHLMWDDIAILARYMLMLSFNNSLDVAAHLPYLFHVVTFLVATGPLSLRASTHGLVINIIHSLCTCSQLHFSEETKQVLRLSLTEFSLPKFYLLFGISKVKSAAVIAFRSSYRDRSFSPGSYERETFALTSLETVTEALLEIMEACMRDIPTCKWLDQWTELAQRFAFQYNPSLQPRALVVFGCISKRVSHGQIKQIIRILSKALESCLKGPDTYNSQVLIEATVIALTKLQPLLNKDSPLHKALFWVAVAVLQLDEVNLYSAGTALLEQNLHTLDSLRIFNDKSPEEVFMAIRNPLEWHCKQMDHFVGLNFNSNFNFALVGHLLKGYRHPSPAIVARTVRILHTLLTLVNKHRNCDKFEVNTQSVAYLAALLTVSEEVRSRCSLKHRKSLLLTDISMENVPMDTYPIHHGDPSYRTLKETQPWSSPKGSEGYLAATYPTVGQTSPRARKSMSLDMGQPSQANTKKLLGTRKSFDHLISDTKAPKRQEMESGITTPPKMRRVAETDYEMETQRISSSQQHPHLRKVSVSESNVLLDEEVLTDPKIQALLLTVLATLVKYTTDEFDQRILYEYLAEASVVFPKVFPVVHNLLDSKINTLLSLCQDPNLLNPIHGIVQSVVYHEESPPQYQTSYLQSFGFNGLWRFAGPFSKQTQIPDYAELIVKFLDALIDTYLPGIDEETSEESLLTPTSPYPPALQSQLSITANLNLSNSMTSLATSQHSPGIDKENVELSPTTGHCNSGRTRHGSASQVQKQRSAGSFKRNSIKKIV

SEQ ID NO: 268 - DNA, 12380 bp, Homo sapiens
  source 1..12380: mol_type = genomic DNA
aatctctagctcgctcgcgctccctctccccgggccgtggaaaggatcccacttccggtggggtgtcatggcggcgtctcggactgtgatggctgtggggagacggcgctagtggggagagcgaccaagaggccccctcccctccccgggtccccttcccctatccccctccccccagcctccttgccaacgccccctttccctctccccctcccgctcggcgctgaccccccatccccacccccgtgggaacactgggagcctgcactccacagaccctctccttgcctcttccctcacctcagcctccgctccccgccctcttcccggcccagggcgccggcccacccttccctccgccgccccccggccgcggggaggacatggccgcgcacaggccggtggaatgggtccaggccgtggtcagccgcttcgacgagcagcttccaataaaaacaggacagcagaacacacataccaaagtcagtactgagcacaacaaggaatgtctaatcaatatttccaaatacaagttttctttggttataagcggcctcactactattttaaagaatgttaacaatatgagaatatttggagaagctgctgaaaaaaatttatatctctctcagttgattatattggatacactggaaaaatgtcttgctgggcaaccaaaggacacaatgagattagatgaaacgatgctggtcaaacagttgctgccagaaatctgccattttcttcacacctgtcgtgaaggaaaccagcatgcagctgaacttcggaattctgcctctggggttttattttctctcagctgcaacaacttcaatgcagtctttagtcgcatttctaccaggttacaggaattaactgtttgttcagaagacaatgttgatgttcatgatatagaattgttacagtatatcaatgtggattgtgcaaaattaaaacgactcctgaaggaaacagcatttaaatttaaagccctaaagaaggttgcgcagttagcagttataaatagcctggaaaaggcattttggaactgggtagaaaattatccagatgaatttacaaaactgtaccagatcccacagactgatatggctgaatgtgcagaaaagctatttgacttggtggatggttttgctgaaagcaccaaacgtaaagcagcagtttggccactacaaatcattctccttatcttgtgtccagaaataatccaggatatatccaaagacgtggttgatgaaaacaacatgaataagaagttatttctggacagtctacgaaaagctcttgctggccatggaggaagtaggcagctgacagaaagtgctgcaattgcctgtgtcaaactgtgtaaagcaagtacttacatcaattgggaagataactctgtcattttcctacttgttcagtccatggtggttgatcttaagaacctgctttttaatccaagtaagccattctcaagaggcagtcagcctgcagatgtggatctaatgattgactgccttgtttcttgctttcgtataagccctcacaacaaccaacactttaagatctgcctggctcagaattcaccttctacatttcactatgtgctggtaaattcactccatcgaatcatcaccaattccgcattggattggtggcctaagattgatgctgtgtattgtcactcggttgaacttcgaaatatgtttggtgaaacacttcataaagcagtgcaaggttgtggagcacacccagcaatacgaatggcaccgagtcttacatttaaagaaaaagtaacaagccttaaatttaaagaaaaacctacagacctggagacaagaagctataagtatcttctcttgtccatggtgaaactaattcatgcagatccaaagctcttgctttgtaatccaagaaaacaggggcccgaaacccaaggcagtacagcagaattaattacagggctcgtccaactggtccctcagtcacacatgccagagattgctcaggaagcaatggaggctctgctggttcttcatcagttagatagcattgatttgtggaatcctgatgctcctgtagaaacattttgggagattagctcacaaatgcttttttacatctgcaagaaattaactagtcatcaaatgcttagtagcacagaaattctcaagtggttgcgggaaatattgatctgcaggaataaatttcttcttaaaaataagcaggcagatagaagttcctgtcactttctccttttttacggggtaggatgtgatattccttctagtggaaataccagtcaaatgtccatggatcatgaagaattactacgtactcctggagcctctctccggaagggaaaagggaactcctctatggatagtgcagcaggatgcagcggaacccccccgatttgccgacaagcccagaccaaactagaagtggccctgtacatgtttctgtggaaccctgacactgaagctgttctggttgccatgtcctgtttccgccacctctgtgaggaagcagatatccggtgtggggtggatgaagtgtcagtgcataacctcttgcccaactataacacattcatggagtttgcctctgtcagcaatatgatgtcaacaggaagagcagcacttcagaaaagagtgatggcactgctgaggcgcattgagcatcccactgcaggaaacactgaggcttgggaagatacacatgcaaaatgggaacaagcaacaaagctaatccttaactatccaaaagccaaaatggaagatggccaggctgctgaaagccttcacaagaccattgttaagaggcgaatgtcccatgtgagtggaggaggatccatagatttgtctgacacagactccctacaggaatggatcaacatgactggcttcctttgtgcccttgggggagtgtgcctccagcagagaagcaattctggcctggcaacctatagcccacccatgggtccagtcagtgaacgtaagggttctatgatttcagtgatgtcttcagagggaaacgcagatacacctgtcagcaaatttatggatcggctgttgtccttaatggtgtgtaaccatgagaaagtgggacttcaaatacggaccaatgttaaggatctggtgggtctagaattgagtcctgctctgtatccaatgctatttaacaaattgaagaataccatcagcaagttttttgactcccaaggacaggttttattgactgataccaatactcaatttgtagaacaaaccatagctataatgaagaacttgctagataatcatactgaaggcagctctgaacatctagggcaagctagcattgaaacaatgatgttaaatctggcaggtatgttcgtgtgcttgggaatatggtccatgcaattcaaataaaaacgaaactgtgtcaattagttgaagtaatgatggcaaggagagatgacctctcattttgccaagagatgaaatttaggaataagatggtagaatacctgacagactgggttatgggaacatcaaaccaagcagcagatgatgatgtaaaatgtcttacaagagatttggaccaggcaagcatggaagcagtagtttcacttctagctggtctccctctgcagcctgaagaaggagatggtgtggaattgatggaagccaaatcacagttatttcttaaatacttcacattatttatgaaccttttgaatgactgcagtgaagttgaagatgaaagtgcgcaaacaggtggcaggaaacgtggcatgtctcggaggctggcatcactgaggcactgtacggtccttgcaatgtcaaacttactcaatgccaacgtagacagtggtctcatgcactccataggcttaggttaccacaaggatctccagacaagagctacatttatggaagttctgacaaaaatccttcaacaaggcacagaatttgacacacttgcagaaacagtattggctgatcggtttgagagattggtggaactggtcacaatgatgggtgatcaaggagaactccctatagcgatggctctggccaatgtggttccttgttctcagtgggatgaactagctcgagttctggttactctgtttgattctcggcatttactctaccaactgctctggaacatgttttctaaagaagtagaattggcagactccatgcagactctcttccgaggcaacagcttggccagtaaaataatgacattctgtttcaaggtatatggtgctacctatctacaaaaactcctggatcctttattacgaattgtgatcacatcctctgattggcaacatgttagctttgaagtggatcctaccaggttagaaccatcagagagccttgaggaaaaccagcggaacctccttcagatgactgaaaagttcttccatgccatcatcagttcctcctcagaattcccccctcaacttcgaagtgtgtgccactgtttataccaggtggttagccagcgtttccctcagaacagcatcggtgcagtaggaagtgccatgttcctcagatttatcaatcctgccattgtctcaccgtatgaagcagggattttagataaaaagccaccacctagaatcgaaaggggcttgaagttaatgtcaaagatacttcagagtattgccaatcatgttctcttcacaaaagaagaacatatgcggcctttcaatgattttgtgaaaagcaactttgatgcagcacgcaggtttttccttgatatagcatctgattgtcctacaagtgatgcagtaaatcatagtctttccttcataagtgacggcaatgtgcttgctttacatcgtctactctggaacaatcaggagaaaattgggcagtatctttccagcaacagggatcataaagctgttggaagacgaccttttgataagatggcaacacttcttgcatacctgggtcctccagagcacaaacctgtggcagatacacactggtccagccttaaccttaccagttcaaagtttgaggaatttatgactaggcatcaggtacatgaaaaagaagaattcaaggctttgaaaacgttaagtattttctaccaagctgggacttccaaagctgggaatcctattttttattatgttgcacggaggttcaaaactggtcaaatcaatggtgatttgctgatataccatgtcttactgactttaaagccatattatgcaaagccatatgaaattgtagtggaccttacccataccgggcctagcaatcgctttaaaacagactttctctctaagtggtttgttgtttttcctggctttgcttacgacaacgtctccgcagtctatatctataactgtaactcctgggtcagggagtacaccaagtatcatgagcggctgctgactggcctcaaaggtagcaaaaggcttgttttcatagactgtcctgggaaactggctgagcacatagagcatgaacaacagaaactacctgctgccaccttggctttagaagaggacctgaaggtattccacaatgctctcaagctagctcacaaagacaccaaagtttctattaaagttggttctactgctgtccaagtaacttcagcagagcgaacaaaagtcctagggcaatcagtctttctaaatgacatttattatgcttcggaaattgaagaaatctgcctagtagatgagaaccagttcaccttaaccattgcaaaccagggcacgccgctcaccttcatgcaccaggagtgtgaagccattgtccagtctatcattcatatccggacccgctgggaactgtcacagcccgactctatcccccaacacaccaagattcggccaaaagatgtccctgggacactgctcaatatcgcattacttaatttaggcagttctgacccgagtttacggtcagctgcctataatcttctgtgtgccttaacttgtacctttaatttaaaaatcgagggccagttactagagacatcaggtttatgtatccctgccaacaacaccctctttattgtctctattagtaagacactggcagccaatgagccacacctcacgttagaatttttggaagagtgtatttctggatttagcaaatctagtattgaattgaaacacctttgtttggaatacatgactccatggctgtcaaatctagttcgtttttgcaagcataatgatgatgccaaacgacaaagagttactgctattcttgacaagctgataacaatgaccatcaatgaaaaacagatgtacccatctattcaagcaaaaatatggggaagccttgggcagattacagatctgcttgatgttgtactagacagtttcatcaaaaccagtgcaacaggtggcttgggatcaataaaagctgaggtgatggcagatactgctgtagctttggcttctggaaatgtgaaattggtttcaagcaaggttattggaaggatgtgcaaaataattgacaagacatgcttatctccaactcctactttagaacaacatcttatgtgggatgatattgctattttagcacgctacatgctgatgctgtccttcaacaattcccttgatgtggcagctcatcttccctacctcttccacgttgttactttcttagtagccacaggtccgctctcccttagagcttccacacatggactggtcattaatatcattcactctctgtgtacttgttcacagcttcattttagtgaagagaccaagcaagttttgagactcagtctgacagagttctcattacccaaattttacttgctgtttggcattagcaaagtcaagtcagctgctgtcattgccttccgttccagttaccgggacaggtcattctctcctggctcctatgagagagagacttttgctttgacatccttggaaacagtcacagaagctttgttggagatcatggaggcatgcatgagagatattccaacgtgcaagtggctggaccagtggacagaactagctcaaagatttgcattccaatataatccatccctgcaaccaagagctcttgttgtctttgggtgtattagcaaacgagtgtctcatgggcagataaagcagataatccgtattcttagcaaggcacttgagagttgcttaaaaggacctgacacttacaacagtcaagttctgatagaagctacagtaatagcactaaccaaattacagccacttcttaataaggactcgcctctgcacaaagccctcttttgggtagctgtggctgtgctgcagcttgatgaggtcaacttgtattcagcaggtaccgcacttcttgaacaaaacctgcatactttagatagtctccgtatattcaatgacaagagtccagaggaagtatttatggcaatccggaatcctctggagtggcactgcaagcaaatggatcattttgttggactcaatttcaactctaactttaactttgcattggttggacaccttttaaaagggtacaggcatccttcacctgctattgttgcaagaacagtcagaattttacatacactactaactctggttaacaaacacagaaattgtgacaaatttgaagtgaatacacagagcgtggcctacttagcagctttacttacagtgtctgaagaagttcgaagtcgctgcagcctaaaacatagaaagtcacttcttcttactgatatttcaatggaaaatgttcctatggatacatatcccattcatcatggtgacccttcctataggacactaaaggagactcagccatggtcctctcccaaaggttctgaaggataccttgcagccacctatccaactgtcggccagaccagtccccgagccaggaaatccatgagcctggacatggggcaaccttctcaggccaacactaagaagttgcttggaacaaggaaaagttttgatcacttgatatcagacacaaaggctcctaaaaggcaagaaatggaatcagggatcacaacaccccccaaaatgaggagagtagcagaaactgattatgaaatggaaactcagaggatttcctcatcacaacagcacccacatttacgtaaagtttcagtgtctgaatcaaatgttctcttggatgaagaagtacttactgatccgaagatccaggcgctgcttcttactgttctagctacactggtaaaatataccacagatgagtttgatcaacgaattctttatgaatacttagcagaggccagtgttgtgtttcccaaagtctttcctgttgtgcataatttgttggactctaagatcaacaccctgttatcattgtgccaagatccaaatttgttaaatccaatccatggaattgtgcagagtgtggtgtaccatgaagaatccccaccacaataccaaacatcttacctgcaaagttttggttttaatggcttgtggcggtttgcaggaccgttttcaaagcaaacacaaattccagactatgctgagcttattgttaagtttcttgatgccttgattgacacgtacctgcctggaattgatgaagaaaccagtgaagaatccctcctgactcccacatctccttaccctcctgcactgcagagccagcttagtatcactgccaaccttaacctttctaattccatgacctcacttgcaacttcccagcattccccaggaatcgacaaggagaacgttgaactctcccctaccactggccactgtaacagtggacgaactcgccacggatccgcaagccaagtgcagaagcaaagaagcgctggcagtttcaaacgtaatagcattaagaagatcgtgtgaagcttgcttgctttcttttttaaaatcaacttaacatgggctcttcactagtgaccccttccctgtccttgccctttccccccatgttgtaatgctgcacttcctgttttataatgaacccatccggtttgccatgttgccagatgatcaactcttcgaagccttgcctaaatttaatgctgccttttctttaactttttttcttctacttttggcgtgtatctggtatatgtaagtgttcagaacaactgcaaagaaagtgggaggtcaggaaacttttaactgagaaatctcaattgtaagagaggatgaattcttgaatactgctactactggccagtgatgaaagccatttgcacagagctctgccttctgtggttttcccttcttcatcctacagagtaaagtgttagtcctatttatacatttttcaagatacaagtttatgagagaaatagtattataaccccagtatgtttaatcttttagctgtggactttttttttaaccgtacaaaactgaaagaaccatagaggtcaagcctcagtgacttgacaccataaagccacagacaaggtacttgggggggagggcagggaaatttcatattttatagtggattcttaagaaatactaacacttgagtattagcaataattacaggaaaataagtgcgaccacatatatcttaacattactgaattaaaactatggcttctaagtccttatccaaactcagtcatccaaactagtttatttttttctccagttgattatcttttaatttttaattttgctaaaggtggtttttttgtgttttgttttttgtaaaccaaaactatactaagtatagtaattatatatatatatatattttttcccctccccctcttctttcctaactaattctgagcagggtaatcagtgaacaaagtgttgaaaattgttcccagaaggtaattttcatagatgtttgcattagctccatagcaaaatggaatggtacgtgacatttagggtagctgatatttttattttgttaaataatttccaagaatagagtatggtgtatattataaatttctttgataagatgtattttgaatgtcttttaatcttcctcctcctctccaaaaaaatcagaaacctctttaagaaaacatgtaggttatatatgctagaattgcatttaatcactgtgaaaagactggtcagcctgcattagtatgacagtaggggggctgttagaattgctgctatactggtggtatggattatcatggcattggaattttcatagtaatgcagatccaatttctttgtggtacctgcagtttacaaaataatttgacttcagtgagcatattggtatctggatgttccaatttagaactaaaccatatttattacaaaaagatattaatccctctactcccaggttccctttatatgttaagatataatggctttgaggggggaaaaaataaacctaggggagaggggagtttcctgtagtgctgtttcattagaggatttcagtaaattaaattccacagctaattcaataaataatggtacatttaagtgttctgattttaataatatatttcacatttatccacacagtaacaatgtaatatgttaatgtaaataaaattggttttgatactcagaaataacaagaatttaattttttaaatttgtttacagtcctgggaaaagtaagaattatttgccaaaataagaggaaagaaaaccttagtattattaatgagtttaccatagaattgttggaaatactgaagacaggtgcaatttactaaacttttgtttttaaactattgtagaggctgcattagaagaaaatgtttataatgacagagcaactatgactatataaaaaagctgaaattagaactgtgtttagaaatagatcagtaacccagtgccaaggatgccaagctgccaccatggtcttggctctcccacaacccagtgtttctggggtaagtttcacagtttctaggccctggaatagcaggcagtgtaagcctttgataactttagttcgatgtttttcttgtttttgtttgttggtttggtgcatatgatagtgggtgttatgctattttgctcttcccatcaaaataaagaaacttccagaggtttactgttaaaaatactgatatttccataaacgggtttaccaagggtgtagtatttcataccgcctgaaatgatcagcattggcacaaatcaaaattcagccgcctttgaaatgcaaaaatacctttgactagtaagtacatcctaggagtttgaaaacttaactaaggtttaaaatttaccttgtttaaagaacttctgacttttgaggaaaatctagctttccaagtaactaaaatgtacatgagataaacctctcaccactatgtgtcccttgagaaatgcaacacttttttagtcttcatacttgtaatctataaaagaaattctgaagtttagaccaagttgcccatttctgcgtaattgacataagttctgttaaaaatattataagtaattcgtttcggtttgtagatgtttcccctgacttgttaaagaggaaaccaggaactcagtcatgtttttgtcctggataatctacctgttatgccagtactcccatccgaggggcatgcccttagttgcccagatggagatgcagttcagtagatttggggcaaagtggctacagctctgtcttccattcactcaacacctgttcatgactgagccaggtgcccaggacacatcctaaacagtcagcttctatcctgtgtcctagttggggagacagagtgccagccagcaaccctcccaggtttgtaggttttaggggttttcagttttgtttgggttttttgttttttgtttttgtttctacatccttccccgactcccaggcataatgaggcatgtcttactcaatgttatgcaatggatttaggcaaaaattcattcttagtgtcagccacacaattttttttaatgcagtatattcacctgtaaatagtttgtgtaaaatttgacaaaaaaagtatatttactatactgtaaatatatgtgatgatatattgtattattttgcttttttgtaaagcagttagttgctgcacatggataacaacaaaaatttgattattctcgtgttagtattgttaacttctttttgcgactgcgttacatcatttaaagaaaatgctgtgtattgtaaacttaaattgtatatgataacttactgtcctttccatccgggcctaaactttggcagttcctttgtctacaaccttgttaatactgtaaacagttgtacgccagcaggaaaaatactgcccaacagacaaaatcgatcattgtaggggaaaatcatagaaatccatttcagatctttattgttcctcaccccattttcctccttgtgtatgtacttcccccacccccctttttttaagtaaaatgtaaattcaatctgctctaagaaaaaaaaaaaaaaaaaaaa

SEQ ID NO: 269 - protein, 543 aa, Homo sapiens
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDELTRHIRIHTGQKPFQCRICMRNFSRSDHLTTHIRTHTGEKPFACDICGRKFARSDERKRHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 270 - DNA, 3137 bp, Homo sapiens
  source 1..3137: mol_type = genomic DNA
gagagatcccagcgcgcagaacttggggagccgccgccgccatccgccgccgcagccagcttccgccgccgcaggaccggcccctgccccagcctccgcagccgcggcgcgtccacgcccgcccgcgcccagggcgagtcggggtcgccgcctgcacgcttctcagtgttccccgcgccccgcatgtaacccggccaggcccccgcaactgtgtcccctgcagctccagccccgggctgcacccccccgccccgacaccagctctccagcctgctcgtccaggatggccgcggccaaggccgagatgcagctgatgtccccgctgcagatctctgacccgttcggatcctttcctcactcgcccaccatggacaactaccctaagctggaggagatgatgctgctgagcaacggggctccccagttcctcggcgccgccggggccccagagggcagcggcagcaacagcagcagcagcagcagcgggggcggtggaggcggcgggggcggcagcaacagcagcagcagcagcagcaccttcaaccctcaggcggacacgggcgagcagccctacgagcacctgaccgcagagtcttttcctgacatctctctgaacaacgagaaggtgctggtggagaccagttaccccagccaaaccactcgactgccccccatcacctatactggccgcttttccctggagcctgcacccaacagtggcaacaccttgtggcccgagcccctcttcagcttggtcagtggcctagtgagcatgaccaacccaccggcctcctcgtcctcagcaccatctccagcggcctcctccgcctccgcctcccagagcccacccctgagctgcgcagtgccatccaacgacagcagtcccatttactcagcggcacccaccttccccacgccgaacactgacattttccctgagccacaaagccaggccttcccgggctcggcagggacagcgctccagtacccgcctcctgcctaccctgccgccaagggtggcttccaggttcccatgatccccgactacctgtttccacagcagcagggggatctgggcctgggcaccccagaccagaagcccttccagggcctggagagccgcacccagcagccttcgctaacccctctgtctactattaaggcctttgccactcagtcgggctcccaggacctgaaggccctcaataccagctaccagtcccagctcatcaaacccagccgcatgcgcaagtaccccaaccggcccagcaagacgcccccccacgaacgcccttacgcttgcccagtggagtcctgtgatcgccgcttctcccgctccgacgagctcacccgccacatccgcatccacacaggccagaagcccttccagtgccgcatctgcatgcgcaacttcagccgcagcgaccacctcaccacccacatccgcacccacacaggcgaaaagcccttcgcctgcgacatctgtggaagaaagtttgccaggagcgatgaacgcaagaggcataccaagatccacttgcggcagaaggacaagaaagcagacaaaagtgttgtggcctcttcggccacctcctctctctcttcctacccgtccccggttgctacctcttacccgtccccggttactacctcttatccatccccggccaccacctcatacccatcccctgtgcccacctccttctcctctcccggctcctcgacctacccatcccctgtgcacagtggcttcccctccccgtcggtggccaccacgtactcctctgttccccctgctttcccggcccaggtcagcagcttcccttcctcagctgtcaccaactccttcagcgcctccacagggctttcggacatgacagcaaccttttctcccaggacaattgaaatttgctaaagggaaaggggaaagaaagggaaaagggagaaaaagaaacacaagagacttaaaggacaggaggaggagatggccataggagaggagggttcctcttaggtcagatggaggttctcagagccaagtcctccctctctactggagtggaaggtctattggccaacaatcctttctgcccacttccccttccccaattactattccctttgacttcagctgcctgaaacagccatgtccaagttcttcacctctatccaaagaacttgatttgcatggattttggataaatcatttcagtatcatctccatcatatgcctgaccccttgctcccttcaatgctagaaaatcgagttggcaaaatggggtttgggcccctcagagccctgccctgcacccttgtacagtgtctgtgccatggatttcgtttttcttggggtactcttgatgtgaagataatttgcatattctattgtattatttggagttaggtcctcacttgggggaaaaaaaaaaaagaaaagccaagcaaaccaatggtgatcctctattttgtgatgatgctgtgacaataagtttgaacctttttttttgaaacagcagtcccagtattctcagagcatgtgtcagagtgttgttccgttaacctttttgtaaatactgcttgaccgtactctcacatgtggcaaaatatggtttggtttttctttttttttttttttgaaagtgttttttcttcgtccttttggtttaaaaagtttcacgtcttggtgccttttgtgtgatgcgccttgctgatggcttgacatgtgcaattgtgagggacatgctcacctctagccttaaggggggcagggagtgatgatttgggggaggctttgggagcaaaataaggaagagggctgagctgagcttcggttctccagaatgtaagaaaacaaaatctaaaacaaaatctgaactctcaaaagtctatttttttaactgaaaatgtaaatttataaatatattcaggagttggaatgttgtagttacctactgagtaggcggcgatttttgtatgttatgaacatgcagttcattattttgtggttctattttactttgtacttgtgtttgcttaaacaaagtgactgtttggcttataaacacattgaatgcgctttattgcccatgggatatgtggtgtatatccttccaaaaaattaaaacgaaaataaagta

SEQ ID NO: 271 - protein, 476 aa, Homo sapiens
MMTAKAVDKIPVTLSGFVHQLSDNIYPVEDLAATSVTIFPNAELGGPFDQMNGVAGDGMINIDMTGEKRSLDLPYPSSFAPVSAPRNQTFTYMGKFSIDPQYPGASCYPEGIINIVSAGILQGVTSPASTTASSSVTSASPNPLATGPLGVCTMSQTQPDLDHLYSPPPPPPPYSGCAGDLYQDPSAFLSAATTSTSSSLAYPPPPSYPSPKPATDPGLFPMIPDYPGFFPSQCQRDLHGTAGPDRKPFPCPLDTLRVPPPLTPLSTIRNFTLGGPSAGVTGPGASGGSEGPRLPGSSSAAAAAAAAAAYNPHHLPLRPILRPRKYPNRPSKTPVHERPYPCPAEGCDRRFSRSDELTRHIRIHTGHKPFQCRICMRNFSRSDHLTTHIRTHTGEKPFACDYCGRKFARSDERKRHTKIHLRQKERKSSAPSASVPAPSTASCSGGVQPGGTLCSSNSSSLGGGPLAPCSSRTRTP

SEQ ID NO: 272 - DNA, 2979 bp, Homo sapiens
  source 1..2979: mol_type = genomic DNA
aactgagcgaggagcaattgattaatagctcggcgaggggactcactgactgttataataacactacaccagcaactcctggcttcccagcagccggaacacagacaggagagagtcagtggcaaatagacatttttcttatttcttaaaaaacagcaacttgtttgctacttttatttctgttgatttttttttcttggtgtgtgtggtggttgtttttaagtgtggagggcaaaaggagataccatcccaggctcagtccaacccctctccaaaacggcttttctgacactccaggtagcgagggagttgggtctccaggttgtgcgaggagcaaatgatgaccgccaaggccgtagacaaaatcccagtaactctcagtggttttgtgcaccagctgtctgacaacatctacccggtggaggacctcgccgccacgtcggtgaccatctttcccaatgccgaactgggaggcccctttgaccagatgaacggagtggccggagatggcatgatcaacattgacatgactggagagaagaggtcgttggatctcccatatcccagcagctttgctcccgtctctgcacctagaaaccagaccttcacttacatgggcaagttctccattgaccctcagtaccctggtgccagctgctacccagaaggcataatcaatattgtgagtgcaggcatcttgcaaggggtcacttccccagcttcaaccacagcctcatccagcgtcacctctgcctcccccaacccactggccacaggacccctgggtgtgtgcaccatgtcccagacccagcctgacctggaccacctgtactctccgccaccgcctcctcctccttattctggctgtgcaggagacctctaccaggacccttctgcgttcctgtcagcagccaccacctccacctcttcctctctggcctacccaccacctccttcctatccatcccccaagccagccacggacccaggtctcttcccaatgatcccagactatcctggattctttccatctcagtgccagagagacctacatggtacagctggcccagaccgtaagccctttccctgcccactggacaccctgcgggtgccccctccactcactccactctctacaatccgtaactttaccctggggggccccagtgctggggtgaccggaccaggggccagtggaggcagcgagggaccccggctgcctggtagcagctcagcagcagcagcagccgccgccgccgccgcctataacccacaccacctgccactgcggcccattctgaggcctcgcaagtaccccaacagacccagcaagacgccggtgcacgagaggccctacccgtgcccagcagaaggctgcgaccggcggttctcccgctctgacgagctgacacggcacatccgaatccacactgggcataagcccttccagtgtcggatctgcatgcgcaacttcagccgcagtgaccacctcaccacccatatccgcacccacaccggtgagaagcccttcgcctgtgactactgtggccgaaagtttgcccggagtgatgagaggaagcgccacaccaagatccacctgagacagaaagagcggaaaagcagtgccccctctgcatcggtgccagccccctctacagcctcctgctctgggggcgtgcagcctgggggtaccctgtgcagcagtaacagcagcagtcttggcggagggccgctcgccccttgctcctctcggacccggacaccttgagatgagactcaggctgatacaccagctcccaaaggtcccggaggccctttgtccactggagctgcacaacaaacactaccaccctttcctgtccctctctccctttgttgggcaaagggctttggtggagctagcactgccccctttccacctagaagcaggttcttcctaaaacttagcccattctagtctctcttaggtgagttgactatcaacccaaggcaaaggggaggctcagaaggaggtggtgtggggacccctggccaagagggctgaggtctgaccctgctttaaagggttgtttgactaggttttgctaccccacttccccttattttgacccatcacaggtttttgaccctggatgtcagagttgatctaagacgttttctacaataggttgggagatgctgatcccttcaagtggggacagcaaaaagacaagcaaaactgatgtgcactttatggcttgggactgatttgggggacattgtacagtgagtgaagtatagcctttatgccacactctgtggccctaaaatggtgaatcagagcatatctagttgtctcaacccttgaagcaatatgtattataaactcagagaacagaagtgcaatgtgatgggaggaacatagcaatatctgctccttttcgagttgtttgagaaatgtaggctattttttcagtgtatatccactcagattttgtgtatttttgatgtacactgttctctaaattctgaatctttgggaaaaaatgtaaagcatttatgatctcagaggttaacttatttaagggggatgtacatatattctctgaaactaggatgcatgcaattgtgttggaagtgtccttggtgccttgtgtgatgtagacaatgttacaaggtctgcatgtaaatgggttgccttattatggagaaaaaaaatcactccctgagtttagtatggctgtatatttctgcctattaatatttggaattttttttagaaagtatatttttgtatgctttgttttgtgacttaaaagtgttacctttgtagtcaaatttcagataagaatgtacataatgttaccggagctgatttgtttggtcattagctcttaatagttgtgaaaaaataaatctattctaacgcaaaaccactaactgaagttcagataatggatggtttgtgactatagtgtaaataaatacttttcaacaata

SEQ ID NO: 273 - protein, 713 aa, Homo sapiens
MGPPSPGDDEEEAAAAAGAPAAAGATGDLASAQLGGAPNRWEVLSATPTTIKDEAGNLVQIPSAATSSGQYVLPLQNLQNQQIFSVAPGSDSSNGTVSSVQYQVIPQIQSADGQQVQIGFTGSSDNGGINQESSQIQIIPGSNQTLLASGTPSANIQNLIPQTGQVQVQGVAIGGSSFPGQTQVVANVPLGLPGNITFVPINSVDLDSLGLSGSSQTMTAGINADGHLINTGQAMDSSDNSERTGERVSPDINETNTDTDLFVPTSSSSQLPVTIDSTGILQQNTNSLTTSSGQVHSSDLQGNYIQSPVSEETQAQNIQVSTAQPVVQHLQLQESQQPTSQAQIVQGITPQTIHGVQASGQNISQQALQNLQLQLNPGTFLIQAQTVTPSGQVTWQTFQVQGVQNLQNLQIQNTAAQQITLTPVQTLTLGQVAAGGAFTSTPVSLSTGQLPNLQTVTVNSIDSAGIQLHPGENADSPADIRIKEEEPDPEEWQLSGDSTLNTNDLTHLRVQVVDEEGDQQHQEGKRLRRVACTCPNCKEGGGRGTNLGKKKQHICHIPGCGKVYGKTSHLRAHLRWHSGERPFVCNWMYCGKRFTRSDELQRHRRTHTGEKKFVCPECSKRFMRSDHLAKHIKTHQNKKGIHSSSTVLASVEAARDDTLITAGGTTLILANIQQGSVSGIGTVNTSATSNQDILTNTEIPLQLVTVSGNETME

SEQ ID NO: 274 - DNA, 11352 bp, Homo sapiens
  source 1..11352: mol_type = genomic DNA
actcagccgtcaccgctcgctctgctggccgctacctgcagcaagatagggccgccatcgccgggcgacgacgaggaggaggcggccgccgcagccggggcccccgccgccgccggagcgacaggtgatttggcttctgcacagttaggaggagcaccaaaccgatgggaggttttgtcagccacacctacaactataaaagatgaagctggtaatctagtccagattccaagtgctgctacttcaagtgggcagtatgttcttccccttcagaatttgcagaatcaacaaatattttccgttgcaccaggatcagattcatcaaatggtacagtgtccagtgttcaattcaagtgataccacagatccagtcagcagatggtcagcaggttcaaattggtttcacaggctcttcagataatgggggtataaatcaagaaagcagtcaaattcagatcattcctggctctaatcaaaccttacttgcctctggaacaccttctgctaacatccagaatctcataccacagactggtcaagtccaggttcagggagttgcaattggtggttcatcttttcctggtcaaacccaagtagttgctaatgtgcctcttggtctgccaggaaatattacgtttgtaccaatcaatagtgtcgatctagattctttgggactctcgggcagttctcagacaatgactgcaggcattaatgccgacggacatttgataaacacaggacaagctatggatagttcagacaattcagaaaggactggtgagcgggtttctcctgatattaatgaaactaatactgatacagatttatttgtgccaacatcctcttcatcacagttgcctgttacgatagatagtacaggtatattacaacaaaacacaaatagcttgactacatctagtgggcaggttcattcttcagatcttcagggaaattatatccagtcgcctgtttctgaagagacacaggcacagaatattcaggtttctacagcacagcctgttgtacagcatctacaacttcaagagtctcagcagccaaccagtcaagcccaaattgtgcaaggtattacaccacagacaatccatggtgtgcaagccagtggtcaaaatatatcacaacaggctttgcaaaatcttcagttgcagctgaatcctggaacctttttaattcaggcacagacagtgaccccttctggacaggtaacttggcaaacgtttcaagtacaaggggtccagaacttgcagaatttgcaaatacagaatactgctgcccaacaaataactttgacgcctgttcaaaccctcacacttggtcaagttgcggcaggtggagccttcacttcaactccagttagtctaagcactggtcagttgccaaatctacaaacagttacagtgaactctatagattctgctggtatacagctacatccaggagagaatgctgacagtcctgcagatattaggatcaaggaagaagaacctgatcctgaagagtggcagctcagtggtgattctaccttgaataccaatgacctaacacacttaagagtacaggtggtagatgaagaaggggaccaacaacatcaagaaggaaaaagacttcggagggtagcttgcacctgtcccaactgtaaagaaggtggtggaagaggtaccaatcttgggaaaaagaagcaacacatttgtcatataccaggatgtggtaaagtctatgggaagacctcacatctgagagctcatctgcgttggcattctggagaacgcccttttgtttgtaactggatgtactgtggtaaaagatttactcgaagtgatgaattacagaggcacagaagaacacatacaggtgagaagaaatttgtttgtccagaatgttcaaaacgctttatgagaagtgaccaccttgccaaacatattaaaacacaccagaataaaaaaggtattcactctagcagtacagtgctggcatctgtggaagctgcgcgagatgatactttgattactgcaggaggaacaacgcttatccttgcaaatattcaacaaggttctgtttcagggataggaactgttaatacttccgccaccagcaatcaagatatccttaccaacactgaaatacctttacagcttgtcacagtttctggaaatgagacaatggagtaaatattacacaaatacttattcattgtggttatttttatacagtagtgagaagaatattgttcctaagttcttagatatctttttattgatgtgcaaaaatttttggattgacagtaacttggttatacatgacactgaaatgccttactttgtatgatattccatagtatattaaaaatggtaaaattgcatgggttttgtaggtacttttggaatctagaagaaatgaaattttaccaagttatataaagagaaaattgaatttaacaatgcgaatggtagtctaaccaaatgcatcaatcctgtgtggtttagtgtaaaaatgagaacatgttggtatttatctattgtaagataaaaaagctggtgggtgaaagaaatcatgttatgataaaaaattttgtaattttcttgatgactggaatttttattatgcataactgacaaatcaagtttccaagcaaatgttacatagtgtaggctttacttagcttatcaatttgtcattttgaagctaattattttaattaggttaactatgtacaatattttaagcattactcttgtaagattttgaaaactacattttaacatggaactctagggatagtcaccttttaaatcctgttgaaaagccatgtttaagatttaatttgccaaaataatgtcttgttaatattctttcaataacgaagttgggcaatataaccaatgtttaaaaaagtttaaaatgtataagttgaggcatttgggtggtaagagaatgttatagtgaattatcccttttcttgactattggaggaccaaaaaaataaggtgtattgcgtcttagcagtgatttttatccaatcttgtttccaaaaaccatgtctcccagggccttaaaagccatcatgtaaattaccagtaaagtgtaacatatgcaaacataacaaaatcacttccatagtgacgatactccaaccatatggatattagtcatagaagaactagaggttttatgatatttttttaagtcttttttttttttgtctaggtagtcagtctgcacttaaatatcaatcattttccttttttgcttcttcccttaaaatttatatgtatccagtacatttaattgagaagcgtatgttttttattatgctgtattttctttttattttttaattattgtttatattttcaattcaaaaatgtacaaaataaagttacattgctggtcttgtaagagctatacagttttcctaaatgtatacctgtaactgcagcagttcacctatttcaaaaatttggaattctgttcatttgttattcttaagaccacctcaaatttaaaggctaccttattgtacgtttaaagtgtattataacagtgtggtagttaataaaacactatttttttttcttttgagtttgttgtattcctatgcataaaaaatattgcagtggtatggggtaagaattggtggtttatttttcttcaacttggctttttatttttagattcttgattttagacactgaattgtaaacaggcatttatttgaagaagagatatatagagacactggtcatttactaattttttacctagagtaaataagaatgagcttattaaataaaatttttgaaaaaaagtcttagcccttagcccacattgattcatatcagttttatcagtaccattttgcaatttttttgttttccgttttaaagcaatgcagaatattttgatttatcgaaaacctgaatttacattaagactcctgaaaatgataagacaagcgttggtaaccatggcaggagttacttgaaaaagttgcctttgaatttgcatgtgtttcatcattataaaggcagagtaggaggaaagagtattaatgtgattgtgtattgtagatgttttaaagtaaaaatcaagtttcttaacacatgtatacagtgggggtaaagatgtccattttctgttttccaggcccagtctgactctgtctgtaatacctgattgcattggagaactctagacacgcataaacatggacagtttttctaaatgtgagacttaagcctgtgatgtaaaataggaagttctacttggaataatataaaggaaccactagaatttacaattattttgaagttacagggattagattttgaatcttaaaatcctttaggtaatttttagaatttttaaattaagattaatgtgaagagaattaagtgagcagcaggtggttcacttgagcaaactgcccattaagtcagattaaaccatttgaatgatagtagagatgttttaagtaatactggatttttacagtaagtttatggtgtactttgaatcgataggtgtccatgcatattttatgaattcgtggagaatgaatgcaatgaagaaagtaagtagtcttgaaaattatttaaataaagttgtagatttttttagtgccccctaggaatatattagtgaactttggaacttttaccaaagttatgtaacctcagtgtagataattttaaatatttctatttttatattttaaaatgttgaatatactctggaaacaacatttgaagatttgctctgatgtcaactttttctggttataaaacctattagtattgtgtataattctcagggtaggtatactctaataggtgttttgtcaattgctttatttttgtaaaggctagagttagtgcatattgaatatatttatgtacaaataattcctgttgtaacatttagtggacgcgattatctgtatacctcaaattttaatttaagaaagtatcacttaaagagcatctcattttctatagattgaggcttaattactgaaaagtgactcaaccaaaaagcacataaccttttaaaggagctacacctaccgcagaaagtcagatgccctgtaaataactttggtctttcaaaatagtggcaatgcttaagatacttaaaaatacacatacatataagctgaaagcatgtcaagcctatttcatagaaaatagttcttaaacagtattgttcattagaaattgctggggagcattttagagattccataggcctggtagccacctcagcttactaaatcaacatctctggagatggagtgtatatgtgttttgaaacagctgctcaagtggttctgttaaacactcctgcgttacaatcacttaaaatagagcaagcatccccttaggctcttaattgtagtttaaattccagtactgcctactcagacccaaaagttttgttttatgaaaaatttgtattgtgttcaatattgtttgaaatttggggttgttgcataaatgattatggaataacatttggttttaaaataatataaactgacatgttatgctacctgttacacaatttggttttcagttttaattatatgaagctggtaacaatcgtttttgttgtaaaagaattatttttcactaaacagtatgtttaaaactgactgccatgaatgagtactaagtcttttgttgtctgacaataagcacaaaacaaattaattgatatctttggtacaaatttgatatttttgtgaaatcacctcataattttatttagtgttgaagaaacaacattggccccttgccttgttcagacagatttgcgtataatgtaaatatatatattagtggcagaaacaaagtactgtagaaacctagaagagggagaattctgcatgaagtctgggaaactcagcagaaatggcgtttgcaaaggtacagtgtggtatgttgggtgatgggggactttgtgagggacagagtggaaaaatttttaagaggggctgccaaattgcaaaagagaagaattttttttgtcagtgattcctagtctttttggaatcctacaccttctccccagaaaaattcattctagtccagggtttattgtttgttttttccctatatttgacagggtctgtctctgtcacacagtgcagtggtgcaatcatggcttactgcagactccacttactggtctcaagcaatcctccccacttcggcctcttaagtagcttggaccacaggtgcacaccaccatgctcagttaattttaatttgttttgtagagatggggtctcactatattgcctaggctggtcttgaattcctgggctcaagtgatccttccacctctgcctcccaaagtgttgggattataggcgtgagccaccacgcttggccaaatctggagtctaaagggtatacaaggtttttttttttcccatgttggcttttatttgatgttataaagtcttcatgatatgaatatattttttaaaaaggtgtcatgtatattgtcatgtaacatgattcatgccacttcattgtattaaatacgtttaagatacagtttttgatgttaagaatgatatcctgagaaggcctatctccttcagtccttttccccatttttcaccttttaactcttgcttctgaatctatgctaagctacttactagatgtgtgatattggtagttttcttaccttaaaacctcattttcatcatccatgaagaggctaacaatagtactccctgttgataggattgttgtattaaaatataagcattcagcatagtgcctgacttaacggtagagattcaaatggtaacctccttccaatgccttcctccccccttaactctggagattccatttttctgtagtgaagtgttttagaattttcattgtttgactatggttcatacattgatttttccaaaatgagaagaagtcttatttctaatgcaaaaatgtgaaaagaacagttggaacctaaagtgatagtggatgaagtgtctgaagtgactgccctatcagaaaaggatggaataagaagaaaagactgaagcacctaaatgtgtattttcttgaggaaattacacccctgtgtaagagtctatcctatttgaacatttctaaaaaccagccgaagaatcttcaggttcattgcgactgaaagataaagtctagcactgaagtggtttttaagattaggaaaggccatcagagaaatgcagttatttctcccctccatccttcccccacaaaaaaaagtctaagcctccgattaatcgacaacaacgaaatacaggaagtattggcttagtgaccttttaggatatctgtccgacttacatcccttctctgagcattactttctgccaccttccgtgaggagctgttcagaaatcagacaaggagtggacatctggtcaaagttgagccaattagatgatttctcctgagaatttggaattggaaaactgatgtcttgggaggtcacatctggaacatacatgggaggcagagaaagcaaagatgacattcaaccccagaagaaaaagaaaatggctttcttgattcctgataacttggcagtgtttgttccagtgtcttgcatttcactatctgcatttcatcctcctggggttccacaagatagccttaaaaccttacagagagactccccaacttagttttttcttaagtcagaaggctgttacttgctaccaaataatgccaactaagacaactgaggtacagtatattctctcgtaaaacaccatagagttgatgacttgatcctatagaaaagggtccccaacccctgggccacagaccggtactggtccctggtctgtgaggaaccacctgggccacagactggtcctggtccctggtctgtgaggaaccaggccatacagcaggaggtaagcgagcattcctgcctgagctctgcctcctgtcagatgagccacaacattagattctcataggagtgcgaaccccgttgtgaaccccgcatgcgagggatctaggtagtgccctccttaagagaatctaatgtctgatgatctgaggtagagcagtttcatcctacaaccatccccaccccccgacccccatccatgaaaaaattgtcttcctcaaaactagtccctggtgccaaaaagtttggggaccgccgtcctaggaaataccaacaaaaagtcacgtttattgcctgccaagtgagtcaggtttagacaaggcagaacttagtatatagcttggatgaatcagaaagggtgggccttgaaagactggtagaatgtgtccaagaaaactgattaaaagccttagaagtccctctacagtttcaggacgccctcttaacagatggctgtatctttatctgtggttggtcttgtggaatactgactttctgccagcattagcctgtgtttctatgtcacaccaagtcacgtatattttcattgtgtgttaggactcagaataggtttgggtctttcttttaattatgtatacccaattacttagtcctcctcacaccctgatcctttaaaaattcttaagttgtgttaattgcatttttctcaattctactcctagtaactctgtgtgtttttttccatctttattcatttagtaaagttgaaacctttcatagcataatatagtgattaagtgcataggtttgggacttagacacacatgggcttgcttgtctcccagttctgccgcttaccacctatgtgacctttcactctttacctttccactcttagcctgttctctaactataaaatgagaatgatgctagcgtcatcttcatagagtgattatggagataaataagttaatgcatgtaaaccactttccataatgcctggcacatattatattatgtattagtaaatgtaagcactggaagaatcagtagtcttcctacacttgaaaatagttgtcttcaactattgttgtattaatgagttaattcaccttgttaagtcaccttgatccacctaattgtcagccaacatttattaacgaaactgctagtattcgtattgtgccttttttattatgaaacatttcaagaatacagaaaaatgcagaaaacaatgtaacaaatacctaagtatctatcacccaacttttatagtcttatttttacctatatattttttcttttagaaaggaatgatcacagttgtgcctgaaatctgtatagccctgtctcatttcatttgtctccctttacccacataagtaattgcagctctgaatttgatgtttatcagaatgataccatgagtggttttatatccttattatatgtatgcatacctaggattgtttttctgattttgaaacttcatataaatggtatcattctgtacaaaaccttctgggacttgttttaccactcatgtttttgagatgtatccacactaccatctttctctaaatcattcatttaaatgctgtagttcattgaataagtttatcacaatgtatccattttcttcttgatgagcatctcagttatttccaatttttgacttataaacaatgctgcaatgaaataattgacatatttccttgtgtacatgtgagcatttcttgagatatgtatctagaacttgaatcactggccaacaggatattcttaatcttcagtttcaccagatacttccaaattgatctccataacatgcataccaaattatattcctaccagccatttataggagttcacattttttccataccctcccaatcctgccttggcagatatgttttttccaatgaataacaatgaaattttgttttgggatttgacaaaatgattctagattcatctggaagaaaagcaagtataagtaagaaatttaaaagggacctgaaaaactaagcaatggatatatttaaaaattggtaccagtagggataaccaaatatttgttaactgtagcagaaacaatgcatgtttttctcactggttccaccttctatctctcagtcaagccctgcccaggtgtggtagtcctcatagtctctcactgtaggggtcttctcacatagtagaccactctcttgagttgaatattgaaaagaaggcttggccgggggcagtggctcatgcctgtggtcccagcactttgggaagctgagtcgagcggattgcttgaggtcagcagttcgagagcagcctgactaacatggtgatgaaaagtacaaaattagctgggggtggtggcgcatgcctgcaatcccagctactcgggaggctgaggcacgagaatcgcttgaacgcaggaggcataggttgcagtgagccgagattgcaccactgcactccagcctgggcgacagagcaagatttcgtctcaaaaaaaaaaaaaaaaaaaaaaaaggcttaagtccaactaactgtttccctgctgttcattttatccaacggctggggatataggctactgcacatgatgggttgatggatgtgaaatgataactcatgactttaattctaatttttctatattctggtgaggttgagcatatttttacaagttcataatccattttcctctgatgggagttgcctgtttgaatcctttgcccacttgctttttggggttatttgcaattttttgcattgatcttcaaacattcttaattaatcccaggtacatattattttatatataagatatattttagagatatataggctgatagtatcttttctcagtctaaggcttatcttttaattttgtttatggtgttttgtgtgtgttgttttgcagaagttttattgtatctgaattgtggcttttgtgtcttaagaaattctttacctactgtcataaaaatttttgtctaaaaattttatgattttgcctttctatttatcttgttaattcactttgaaattacttttgtttatggcataaagtccattattttccatatggataatcagttgtcttagcagtttattgaatggtccaacgtttccctgctgattataatgccctctctgttatttatcctaaattcttgacatccggtaaggaagtctcctctgcccacccttctctgtatttcaaaatggttttgactgttcttgaccccttgctctttctgtgaactttaggattaaatgattatgtagaattaaacaatcgggttaaggttttttattagaattacattgaatctatagattaatttggggaaagaatgttatctttctatccatgataattctgtatctctccattaattcaagtctttaatggtttttaaaaataaattattgtctctttgaaaa

SEQ ID NO: 275 - protein, 284 aa, Homo sapiens
MSMLPSFGFTQEQVACVCEVLQQGGNLERLGRFLWSLPACDHLHKNESVLKAKAVVAFHRGNFRELYKILESHQFSPHNHPKLQQLWLKAHYVEAEKLRGRPLGAVGKYRVRRKFPLPRTIWDGEETSYCFKEKSRGVLREWYAHNPYPSPREKRELAEATGLTTTQVSNWFKNRRQRDRAAEAKERENTENNNSSSNKQNQLSPLEGGKPLMSSSEEEFSPPQSPDQNSVLLLQGNMGHARSSNYSLPGLTASQPSHGLQTHQHQLQDSLLGPLTSSLVDLGS

SEQ ID NO: 276 - DNA, 4005 bp, Homo sapiens
  source 1..4005: mol_type = genomic DNA
aagttccccggcaactagcagcatccaccgggcgggaggtcggaggcagcaaggccttaaaggctactgagtgcgccggccgttccgtgtccagaacctcccctactcctccgccttctcttccttggccgcccaccgccaagttccgactccggttttcgcctttgcaaagcctaaggaggaggttaggaacagccgcgcccccctccctgcggccgccgccccctgcctctcggctctgctccctgccgcgtgcgcctgggccgtgcgccccggcaggccccagccatgtcgatgctgccgtcgtttggctttacgcaggagcaagtggcgtgcgtgtgcgaggttctgcagcaaggcggaaacctggagcgcctgggcaggttcctgtggtcactgcccgcctgcgaccacctgcacaagaacgagagcgtactcaaggccaaggcggtggtcgccttccaccgcggcaacttccgtgagctctacaagatcctggagagccaccagttctcgcctcacaaccaccccaaactgcagcaactgtggctgaaggcgcattacgtggaggccgagaagctgcgcggccgacccctgggcgccgtgggcaaatatcgggtgcgccgaaaatttccactgccgcgcaccatctgggacggcgaggagaccagctactgcttcaaggagaagtcgaggggtgtcctgcgggagtggtacgcgcacaatccctacccatcgccgcgtgagaagcgggagctggccgaggccaccggcctcaccaccacccaggtcagcaactggtttaagaaccggaggcaaagagaccgggccgcggaggccaaggaaagggagaacaccgaaaacaataactcctcctccaacaagcagaaccaactctctcctctggaagggggcaagccgctcatgtccagctcagaagaggaattctcacctccccaaagtccagaccagaactcggtccttctgctgcagggcaatatgggccacgccaggagctcaaactattctctcccgggcttaacagcctcgcagcccagtcacggcctgcagacccaccagcatcagctccaagactctctgctcggccccctcacctccagtctggtggacttggggtcctaagtggggagggactggggcctcgaagggattcctggagcagcaaccactgcagcgactagggacacttgtaaatagaaatcaggaacatttttgcagcttgtttctggagttgtttgcgcataaaggaatggtggactttcacaaatatctttttaaaaatcaaaaccaacagcgatctcaagcttaatctcctcttctctccaactctttccacttttgcattttccttcccaatgcagagatcagggaaaaaaaaaaaaaaaaacccaaacaaacaaaagcacccaggcacccagtctgagttctgggcaactgatacgcctgtttcagcagcctttcttttttttcaatgaatgggaattgcaaatcaactggattttcattatttccttttaatttatatatggagaaatgtgaagagggaaaggaaatggaaagagaaagagaaagggagataaaaatagtgaaaataagagcctccaggctcagaagaactgattacattcttaaggtgaacaggaaaaatacaatctataactttctttgatgaggaaaaattaagtttacatttttcatatttagtgttaaacaatttaatgtagattaaaataaaagaccagtattaggaggaaaaaacaagtgcctaaatgtcttaatgctctctatgtgagacagaaatagacgtgaccattagtaatgcaactatttttgtcaaatttagtgggattttttggttgttgtttgttttcttgggttttttttttttaaatgacaaactctaaaaatgtaccaatgtgaaaaaacactttcctgaatgccattactcatgccctcaaagctttcatatctgtagcctactcctgtaaagggtttctcctgtttctagtttctagtttgcaaaggtatgccaacgaatctggcaacctggtatttgttactaaaacagcatgtgttttcaggtttcttttctattgtacctaaagcagtctaaattaaaacttagtagaacaccaggagtatgattctgtttctgaaaggtgagtggtgtattgctgtcattgggccctatttttttttttaaatatatttttctttcttacttaatggtggctgtgaattgcagggtactttgaaggccatcatctgaaccaagagtagtaactagattaattatatgacagaaagagtgaatttagccttggggtatttattaacttctattatttagatatgcaattttgtttaccactatctcttcacagcattcatatgttaactaagctcttttgtgttaacaagtttatgacaagactgtgaaagtaaaaataatttatctgcttgaagacaaaaaagggaaggagaacaaggatagaaacattgtgaattaatttgtacaaatagaaagcagaccagcaggacaggagctcttttgcagtgctgccggatggtgtctagaaaaatcccagtaatcatgtaggctccatattatttttgcctggggcaaaatgatgtatcttctgtatttagcttttaaaattagtgaaacaaatggcattatttattaaaattctactcaggataacaggattggcttgcttgtgctttgtaaaatattgttccccagggagaatctatttattttctgacatgacagtttcataattttgattttttcccatctataattgtcaccattaatatattatttctttcttactctttcctatttctgttctggtctagaaaactgaggtgtgcttggggaaatcaaggccttattttttaaaactgtcaaatagctttgagttccagggagaaatggtcactgtacttaactgtacacacttttacttataaagtcgtttttccccctcagtgatggacagatgtgcacacagccactcagagaccatcatttggtttgggagctcagggtcccaatctcccttgttagtgttttggatcattaagtcggtgtttatttaagtcactagggtgtttattctccctgtcttgggccacggagaaatgccaccctctgcaggaggggctctcacagggaatctcctcacattcttcacattcactccccaaaacaaacaccctgcacaaagatagctttcaatgaccctgacaggcttcaaaggacgctgcggaaatctctctggaaaataggaagggccaattactttaatttcttacatgccgtctcttcctactccggtgactaccctccactctcccagctccgcctgggataattttaggctcgtggaagttgtgtctcgcagggcgccccggcccacttcctgctcccttagttggtgaatcttggaaatgtctgcagatccggcgaggagcaagagcgctggtcctgtaaaagcccccgtgattactgcttgtaaacttgttaaaaggacaattttctgtcacagtcataaactctttgtaaaacccctggcactaaatccagagcgcgcatattccagatgtgtttaataagaaattgcacaatgtgcctccttccgccatcctcggctcagtctgggtaaaaggggcgagcaggaggaggcgagaaagctggatattgctttgagtttttttggcaatcatttcagagagatcattaggggaaactaaccatatttttttcttccctcgaggaaatcttcgagaaccagctgttaagggacctccagtttaccttctgggcgtcgcagtttaactgaatctaaagccacttttcgtttctcctctacagatgtcataacggtagccaaaagtaatgaacaacggcttataaatagtttgtaggacgaaaccatacagcattgtgccaagtgaaatgaaaaaaaaaaaaaa

SEQ ID NO: 277 - protein, 291 aa, Homo sapiens
MSMLPTFGFTQEQVACVCEVLQQGGNIERLGRFLWSLPACEHLHKNESVLKAKAVVAFHRGNFRELYKILESHQFSPHNHAKLQQLWLKAHYIEAEKLRGRPLGAVGKYRVRRKFPLPRSIWDGEETSYCFKEKSRSVLREWYAHNPYPSPREKRELAEATGLTTTQVSNWFKNRRQRDRAAEAKERENNENSNSNSHNPLNGSGKSVLGSSEDEKTPSGTPDHSSSSPALLLSPPPPGLPSLHSLGHPPGPSAVPVPVPGGGGADPLQHHHGLQDSILNPMSANLVDLGS

SEQ ID NO: 278 - DNA, 2206 bp, Homo sapiens
  source 1..2206: mol_type = genomic DNA
aagaaagctgagagccagctagacgggagggagaacgagtgagaagcgagcgagggaacggcgggcgggcgcggagcatgcggagcggcgccccgggcggcccccgggcttgggcgagggcttgggccaggcgcgcgggccgttggggttcggagcttcgtgggacccgcggccggcgcggggacgtacggcagtgactcggggctcaccgggggccagtgccgggccagggggccagccccgcccgcgtctcggcccggacggcccggcgaggaagctcccatgcgggaccgcgcggcccggtgagggcgcgcgcgggcgggcggggacgcagccggcaccatgtccatgctgcccaccttcggcttcacgcaggagcaagtggcgtgcgtgtgcgaggtgctgcagcagggcggcaacatcgagcggctgggccgcttcctgtggtcgctgcccgcctgcgagcaccttcacaagaatgaaagcgtgctcaaggccaaggccgtggtggccttccaccgcggcaacttccgcgagctctacaagatcctggagagccaccagttctcgccgcacaaccacgccaagctgcagcagctgtggctcaaggcacactacatcgaggcggagaagctgcgcggccgacccctgggcgccgtgggcaaataccgcgtgcgccgcaaattcccgctgccgcgctccatctgggacggcgaggagaccagctactgcttcaaggaaaagagtcgcagcgtgctgcgcgagtggtacgcgcacaacccctacccttcaccccgcgagaagcgtgagctggcggaggccacgggcctcaccaccacacaggtcagcaactggttcaagaaccggcggcagcgcgaccgggcggccgaggccaaggaaagggagaacaacgagaactccaattctaacagccacaacccgctgaatggcagcggcaagtcggtgttaggcagctcggaggatgagaagactccatcggggacgccagaccactcatcatccagccccgcactgctcctcagcccgccgccccctgggctgccgtccctgcacagcctgggccaccctccgggccccagcgcagtgccagtgccggtgccaggcggaggtggagcggacccactgcaacaccaccatggcctgcaggactccatcctcaaccccatgtcagccaacctcgtggacctgggctcctagaacccatttgccttgatgagcttgccttttgtgacttgacactggggacgtggagtggcggtgtccaggggcgccccgcccctgcggccccaccaggtactgaaagacccgcaggctgagcgggtagaacagccgggtagggcagatagctgtctatgttggttcttgtttgggatttattttcaacaagttacttttaggatccttttggggctggagactgagtcttgaaccacagaagggaataaattatacaccactgtcattctctctctccctctgtctcttccttttaccctctcttgtcttgccttttccccctttcctcttcctttcccttccttctcttttcttttttctgctttctgtctttctccctctccttgtattgctttccttctagatttctagcttgccaccgttcattctctccttctgtctctccctttctctctccttctctgtttctcctctcttctctcctgccagtctcttgtactctgtgtcctggtccctccgtatgtacccctgtctttctcctcctgactggtggtctatctgcccctacctctggccctcgctttaccggagtagggggtgggagagggaagaggagagaaaatacagggactttgaacctaggccatctcctgaggccttttccctcgcccatgtgggtcagtgggagctgcaggtgtcagcttttcgtctagtaacttaagtgagagagaaagggcagcgccacagaagcccctaaacgccgcctcgtcatacgcccctcctccttctctcttggcgaggccccgccacaccgcgctcttcctcccgggactgtgactacagcgctcccggctgagcgcgccccccgagccgccgacttgccgtctccccgtaatgccctcatgtgaatgttcttcgggaaatatttctgcttttattttataataaaattagaaatcataaatatataaatggttatatgccacaa

SEQ ID NO: 279 - protein, 592 aa, Homo sapiens
MEMQDLTSPHSRLSGSSESPSGPKLGNSHINSNSMTPNGTEVKTEPMSSSETASTTADGSLNNFSGSAIGSSSFSPRPTHQFSPPQIYPSNRPYPHILPTPSSQTMAAYGQTQFTTGMQQATAYATYPQPGQPYGISSYGALWAGIKTEGGLSQSQSPGQTGFLSYGTSFSTPQPGQAPYSYQMQGSSFTTSSGIYTGNNSLTNSSGFNSSQQDYPSYPSFGQGQYAQYYNSSPYPAHYMTSSNTSPTTPSTNATYQLQEPPSGITSQAVTDPTAEYSTIHSPSTPIKDSDSDRLRRGSDGKSRGRGRRNNNPSPPPDSDLERVFIWDLDETIIVFHSLLTGSYANRYGRDPPTSVSLGLRMEEMIFNLADTHLFFNDLEECDQVHIDDVSSDDNGQDLSTYNFGTDGFPAAATSANLCLATGVRGGVDWMRKLAFRYRRVKEIYNTYKNNVGGLLGPAKREAWLQLRAEIEALTDSWLTLALKALSLIHSRTNCVNILVTTTQLIPALAKVLLYGLGIVFPIENIYSATKIGKESCFERIIQRFGRKVVYVVIGDGVEEEQGAKKHAMPFWRISSHSDLMALHHALELEYL

SEQ ID NO: 280 - DNA, 4163 bp, Homo sapiens
  source 1..4163: mol_type = genomic DNA
ctcagatgctatctgccgctgctgtttggtggggaaggagcgctgggcgcaaagctgttaccaaacagaacggtgggagctgatggctccgagtttggggcgaggtagaaactctccagtgccacttccgactttaagccttcctgttgccgtccactgtggcgggtttcttcctggggaacacgttttcgctcagtcgctcggcagcccgagcctgcggcagcggccaggcgcctgccccctgcgccgagctttcccctgcagaggcgctccactcccagaagcgccgcggctgcaccagagcgcctgagagcccccgcgcgtacccatccaggagcaaaactatgtcaggaatggaggtttgctaacccagaaaattcgaaggaacacattaaactggtggatgcagcagatgtaagcgctgtgcaaacatctcaagccagttcagatgttgctgtttcctcaagttgcaggtctatggaaatgcaggatctaaccagcccgcatagccgtctgagtggtagtagtgaatcccccagtggccccaaactcggtaactctcatataaatagtaattccatgactcccaatggcaccgaagttaaaacagagccaatgagcagcagtgaaacagcttcaacgacagccgacgggtctttaaacaatttctcaggttcagcaattgggagcagtagtttcagcccacgaccaactcaccagttctctccaccacagatttacccttccaacagaccatacccacatattctccctaccccttcctcacaaactatggctgcatatgggcaaacacagtttaccacaggaatgcaacaagctacagcctatgccacgtacccacagccaggacagccgtacggcatttcctcatatggtgcattgtgggcaggcatcaagactgaaggtggattgtcacagtctcagtcacctggacagacaggatttctcagctatggcacaagcttcagtacccctcaacctggacaggcaccatacagctaccagatgcaaggtagcagttttacaacatcatcaggaatatatacaggaaataattcactcacaaattcctctggatttaatagttcacagcaggactatccgtcttatcccagttttggccagggtcagtacgcacagtattataacagctcaccgtatccagcacattatatgaccagcagcaacaccagcccaacgacaccatccaccaatgccacttaccagcttcaagaaccgccatctggcatcaccagccaagcagttacagatcccacagcagagtacagcacaatccacagcccatcaacacccattaaagattcagattctgatcgattgcgtcgaggttcagatgggaaatcacgtggacggggccgaagaaacaataatccttcacctcccccagattctgatcttgagagagtgttcatctgggacttggatgagacaatcattgttttccactccttgcttactgggtcctacgccaacagatatgggagggatccacccacttcagtttcccttggactgcgaatggaagaaatgattttcaacttggcagacacacatttattttttaatgacttagaagaatgtgaccaagtccatatagatgatgtttcttcagatgataacggacaggacctaagcacatataactttggaacagatggctttcctgctgcagcaaccagtgctaacttatgtttggcaactggtgtacggggcggtgtggactggatgagaaagttggccttccgctacagacgggtaaaagagatctacaacacctacaaaaataatgttggaggtctgcttggtccagctaagagggaagcctggctgcagttgagggccgaaattgaagccctgaccgactcctggttgacactggccctgaaagcactctcgctcattcactcccggacaaactgtgtgaatattttagtaacaactactcagctcatcccagcattggcgaaagtcctgctgtatgggttaggaattgtatttccaatagaaaatatttacagtgcaactaaaataggaaaagaaagctgttttgagagaataattcaaaggtttggaagaaaagtggtgtatgttgttataggagatggtgtagaagaagaacaaggagcaaaaaagcacgcgatgcccttctggaggatctccagccactcggacctcatggccctgcaccatgccttggaactggagtacctgtaacagcgctcggcactttgacagcgcacagctgctctgtgaccagggacagatccagcaggccccagtctcgcatcagcgccggcctccagaacttagcaatttccgcctggtgatgcgcagttgctgtcagtcttgacctctgcctttgtggtgaatggaggaccacgtctatttcatcagaacagctgttgactctagtactgtgaatccagtgaaaataagccatgagaatgttttagcacagcgttatgtgtctgccacattaactacacggttcaaacctgtgaagaaaggacctgcaaacgcttcagttgttagcattttcaatgtgatataaacagcttctccaatacagcaaacctaattgcacaacagagactgaaatgtgtttcctgaataccagtggaggaattttcttgtaaagaaggtttactttttggtgtctcatacccagggtaatctgtacatctctacttatttatgaacagactttttttaaaaagataaaaaaacagctttattgaggtataattcacccaccagacttttttaaacatcaaataattgaagagacaatagcattagaaataagtgattaaaggcctctgcctcacaacatggcaagtacagtactttgaattttagcacattgcatagtagttttaagtatgtctaatttaaacgtataatatgtacatcactgagacaatcatgtacagaaagaatttttggtgtaaatttgtaataatggataattcttttacatattgtttagggaaatgatattgaaaggtagcaatgcctggatagtgaagcatgaggcagcacgtgcacaaattcatgtgccgtgccttatctgagttttcggtataaatatgtagataatggattttttttttttagataatgttgtcaagaccaaaagcatggatgtcaagtgtcagtaaggattttgttttctaaaattttttcctgcatcagttcttctgagggccttgatgaaataacacagcagtttcttaaacaatttgaaacaaaatgagctctcctaccacctcactttttcatttccacactaatgtattatatgtaactacttggaaaaaataattattcaaatgcttcttcccacaaagaatatagatgatagtagatatattttattaataaaatggttcatgaatcggagactaacaaagttttcatgtgctcagaattattaattatcgtgtctgcattttctttcgataaaggaagacacacgatgctaatccggaaatcagcaaactttgcattactccctatgtgcgtattttctctttcttcctgtcaccctgaggaaggttcattgccattgtcatcaccatggaaacaacgttcctctccacctgcattatgtactacatgacaggcatcaatctggggaaataataaaattatcacctttgtcagaccataagagtttctccaaaagtggtcagtttggctgggcaatattttctctcatctaacaaacacaatccattgtcatgaaattacccttaggatgagtcttctttaatcaatcatatattgggcgggaaaaacaccagctttgacccgaagtagttgaagagctacttcattcttttctgaagttgtgtgttgctgctagaaatagtcatttgtgaattatccaaattgtttaaattcacaattgaattagttttttcttcctttttgcttgaagcaaacagttgacaatttttaaccttttcattttatgtttttgtactctgcagactgaaaagacaaagtttatcttggccttactgtataaaggtgtgctgtgtccaccgttgtgtacagaatttttcttcattaattttgtgtttaagttaataaaatttatttgtgatgtactgtaa

SEQ ID NO: 281 - protein, 538 aa, Homo sapiens
MVELVISPSLTVNSDCLDKLKFNRADAAVWTLSDRQGITKSAPLRVSQLFSRSCPRVLPRQPSTAMAAYGQTQYSAGIQQATPYTAYPPPAQAYGIPSYSIKTEDSLNHSPGQSGFLSYGSSFSTSPTGQSPYTYQMHGTTGFYQGGNGLGNAAGFGSVHQDYPSYPGFPQSQYPQYYGSSYNPPYVPASSICPSPLSTSTYVLQEASHNVPNQSSESLAGEYNTHNGPSTPAKEGDTDRPHRASDGKLRGRSKRSSDPSPAGDNEIERVFVWDLDETIIIFHSLLTGTFASRYGKDTTTSVRIGLMMEEMIFNLADTHLFFNDLEDCDQIHVDDVSSDDNGQDLSTYNFSADGFHSSAPGANLCLGSGVHGGVDWMRKLAFRYRRVKEMYNTYKNNVGGLIGTPKRETWLQLRAELEALTDLWLTHSLKALNLINSRPNCVNVLVTTTQLIPALAKVLLYGLGSVFPIENIYSATKTGKESCFERIMQRFGRKAVYVVIGDGVEEEQGAKKHNMPFWRISCHADLEALRHALELEYL

SEQ ID NO: 282 - DNA, 2483 bp, Homo sapiens
  source 1..2483: mol_type = genomic DNA
agcagccgcggcagcccaggaggcggaggcagcggcaacggcagagacagcaacgtgcccgccgcagtcagcccggcctcgtcggacccgcaccggcccgcccgcccgcccgcaccgcgtcggggcgccctctccactgcgcgcggtacaaggaaatggtagaactagtgatctcacccagcctcactgtaaacagcgattgtctggataaactgaagtttaaccgtgctgacgctgctgtgtggactctgagtgacagacaaggcatcaccaaatcggcccccctgagagtgtcccagctcttctccagatcttgcccacgtgtcctcccccgccagccttccacagccatggcagcctacggccagacgcagtacagtgcggggatccagcaggctaccccctatacagcttacccacctccagcacaagcctatggaatcccttcctacagcatcaagacagaagacagcttgaaccattcccctggccagagtggattcctcagctatggctccagcttcagcacctcacccactggacagagcccatacacctaccagatgcacggcacaacagggttctatcaaggaggaaatggactgggcaacgcagccggtttcgggagtgtgcaccaggactatccttcctaccccggcttcccccagagccagtacccccagtattacggctcatcctacaaccctccctacgtcccggccagcagcatctgcccttcgcccctctccacgtccacctacgtcctccaggaggcatctcacaacgtccccaaccagagttccgagtcacttgctggtgaatacaacacacacaatggaccttccacaccagcgaaagagggagacacagacaggccgcaccgggcctccgacgggaagctccgaggccggtctaagaggagcagtgacccgtccccggcaggggacaatgagattgagcgtgtgttcgtgtgggacttggatgagacaataattatttttcactccttactcacggggacatttgcatccagatacgggaaggacaccacgacgtccgtgcgcattggccttatgatggaagagatgatcttcaaccttgcagatacacatctgttcttcaatgacctggaggattgtgaccagatccacgttgatgacgtctcatcagatgacaatggccaagatttaagcacatacaacttctccgctgacggcttccacagttcggccccaggagccaacctgtgcctgggctctggcgtgcacggcggcgtggactggatgaggaagctggccttccgctaccggcgggtgaaggagatgtacaatacctacaagaacaacgttggtgggttgataggcactcccaaaagggagacctggctacagctccgagctgagctggaagctctcacagacctctggctgacccactccctgaaggcactaaacctcatcaactcccggcccaactgtgtcaatgtgctggtcaccaccactcaactaattcctgccctggccaaagtcctgctatatggcctggggtctgtgtttcctattgagaacatctacagtgcaaccaagacagggaaggagagctgcttcgagaggataatgcagagattcggcagaaaagctgtctacgtggtgatcggtgatggtgtggaagaggagcaaggagcgaaaaagcacaacatgcctttctggcggatatcctgccacgcagacctggaggcactgaggcacgccctggagctggagtatttatagcaggatcagcagcatctccacctgccatctcaccctcagaccccctcgccttccccacctccccaccgagaactccagagacccagatgttggacaccaggaaggggccccacagccgagacgacgtgtccagtgaccatctcagaagccgtccatcagtccaaatgggggttctgagaaggaaagtacccaacattggcttcggagtatttgactttggggaaaagggctggctcggagtctagactcttctgtaagactcacagaacaaaagcaaggaattgctgatttggggggtgcctggtgatgaggaggggatgggtttgtcttgtcttctttttaatttatggactagtctcattactccggaattatgctcttgtacctgtgtggctgggtttcttagtcgttggtttggtttggttttttgaactggtatgtggggtggttcacagttctaatgtaagcactctattctccaagttgtgctttgtggggacaatcattctttgaacattagagaggaaggcagttcaagctgttgaaaagactattgcttatttttgtttttaaagacctacttgacgtcatgtggacagtgcacgtgccttacgctacatcttgttttctaggaagagggggatgctgggaaggaatgggtgctttgtgatggataaaaggcattaaataaaaccacgtttacattttgaa

SEQ ID NO: 283 - protein, 484 aa, Homo sapiens
MERRMKAGYLDQQVPYTFSSKSPGNGSLREALIGPLGKLMDPGSLPPLDSEDLFQDLSHFQETWLAEAQVPDSDEQFVPDFHSENLAFHSPTTRIKKEPQSPRTDPALSCSRKPPLPYHHGEQCLYSSAYDPPRQIAIKSPAPGALGQSPLQPFPRAEQRNFLRSSGTSQPHPGHGYLGEHSSVFQQPLDICHSFTSQGGGREPLPAPYQHQLSEPCPPYPQQSFKQEYHDPLYEQAGQPAVDQGGVNGHRYPGAGVVIKQEQTDFAYDSDVTGCASMYLHTEGFSGPSPGDGAMGYGYEKPLRPFPDDVCVVPEKFEGDIKQEGVGAFREGPPYQRRGALQLWQFLVALLDDPTNAHFIAWTGRGMEFKLIEPEEVARLWGIQKNRPAMNYDKLSRSLRYYYEKGIMQKVAGERYVYKFVCEPEALFSLAFPDNQRPALKAEFDRPVSEEDTVPLSHLDESPAYLPELAGPAQPFGPKGGYSY

SEQ ID NO: 284 - DNA, 2335 bp, Homo sapiens
  source 1..2335: mol_type = genomic DNA
gctcacaactgtctgctgcgcccgaaaaacaagtcggtgcgctggggacccggggccggggccgccttactccggcctagccccgcggccctcggtgcgggctccagggcatgctcgggaccccccgcggctccagcccagacgccccggcctcaggtctcggcccccgcttggggccccggccgtgcggccggagggagcggccggatggagcggaggatgaaagccggatacttggaccagcaagtgccctacaccttcagcagcaaatcgcccggaaatgggagcttgcgcgaagcgctgatcggcccgctggggaagctcatggacccgggctccctgccgcccctcgactctgaagatctcttccaggatctaagtcacttccaggagacgtggctcgctgaagctcaggtaccagacagtgatgagcagtttgttcctgatttccattcagaaaacctagctttccacagccccaccaccaggatcaagaaggagccccagagtccccgcacagacccggccctgtcctgcagcaggaagccgccactcccctaccaccatggcgagcagtgcctttactccagtgcctatgacccccccagacaaatcgccatcaagtcccctgcccctggtgcccttggacagtcgcccctacagccctttccccgggcagagcaacggaatttcctgagatcctctggcacctcccagccccaccctggccatgggtacctcggggaacatagctccgtcttccagcagcccctggacatttgccactccttcacatctcagggagggggccgggaacccctcccagccccctaccaacaccagctgtcggagccctgcccaccctatccccagcagagctttaagcaagaataccatgatcccctgtatgaacaggcgggccagccagccgtggaccagggtggggtcaatgggcacaggtacccaggggcgggggtggtgatcaaacaggaacagacggacttcgcctacgactcagatgtcaccgggtgcgcatcaatgtacctccacacagagggcttctctgggccctctccaggtgacggggccatgggctatggctatgagaaacctctgcgaccattcccagatgatgtctgcgttgtccctgagaaatttgaaggagacatcaagcaggaaggggtcggtgcatttcgagaggggccgccctaccagcgccggggtgccctgcagctgtggcaatttctggtggccttgctggatgacccaacaaatgcccatttcattgcctggacgggccggggaatggagttcaagctcattgagcctgaggaggtcgccaggctctggggcatccagaagaaccggccagccatgaattacgacaagctgagccgctcgctccgatactattatgagaaaggcatcatgcagaaggtggctggtgagcgttacgtgtacaagtttgtgtgtgagcccgaggccctcttctctttggccttcccggacaatcagcgtccagctctcaaggctgagtttgaccggcctgtcagtgaggaggacacagtccctttgtcccacttggatgagagccccgcctacctcccagagctggctggccccgcccagccatttggccccaagggtggctactcttactagcccccagcggctgttccccctgccgcaggtgggtgctgccctgtgtacatataaatgaatctggtgttggggaaaccttcatctgaaacccacagatgtctctggggcagatccccactgtcctaccagttgccctagcccagactctgagctgctcaccggagtcattgggaaggaaaagtggagaaatggcaagtctagagtctcagaaactcccctgggggtttcacctgggccctggaggaattcagctcagcttcttcctaggtccaagccccccacaccttttccccaaccacagagaacaagagtttgttctgttctgggggacagagaaggcgcttcccaacttcatactggcaggagggtgaggaggttcactgagctccccagatctcccactgcggggagacagaagcctggactctgccccacgctgtggccctggagggtcccggtttgtcagttcttggtgctctgtgttcccagaggcaggcggaggttgaagaaaggaacctgggatgaggggtgctgggtataagcagagagggatgggttcctgctccaagggaccctttgcctttcttctgccctttcctaggcccaggcctgggtttgtacttccacctccaccacatctgccagaccttaataaaggcccccacttctccca

SEQ ID NO: 285 - protein, 1548 aa, Homo sapiens
MPSDFISLLSADLDLESPKSLYSRDSLKLHPSQNFHRAGLLEESVYDLLPKELQLPPSRETSVASMSQTSGGEAGSPPPAVVAADASSAPSSSSMGGACSSFTTSSSPTIYSTSVTDSKAMQVESCSSAVGVSNRGVSEKQLTSNTVQQHPSTPKRHTVLYISPPPEDLLDNSRMSCQDEGCGLESEQSCSMWMEDSPSNFSNMSTSSYNDNTEVPRKSRKRNPKQRPGVKRRDCEESNMDIFDADSAKAPHYVLSQLTTDNKGNSKAGNGTLENQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTPCKEVDIEGTTVIEVGLDPSNNMTLAVDCVGILKLRNADVEARIGIAGSKKKSTRARLVFRVNIMRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVSVGIYVVTNAGRSHDVQPFTYTPDPAAGALNVNVKKEISSPARPCSFEEAMKAMKTTGCNLDKVNIIPNALMTPLIPSSMIKSEDVTPMEVTAEKRSSTIFKTTKSVGSTQQTLENISNIAGNGSFSSPSSSHLPSENEKQQQIQPKAYNPETLTTIQTQDISQPGTFPAVSASSQLPNSDALLQQATQFQTRETQSREILQSDGTVVNLSQLTEASQQQQQSPLQEQAQTLQQQISSNIFPSPNSVSQLQNTIQQLQAGSFTGSTASGSSGSVDLVQQVLEAQQQLSSVLFSAPDGNENVQEQLSADIFQQVSQIQSGVSPGMFSSTEPTVHTRPDNLLPGRAESVHPQSENTLSNQQQQQQQQQQVMESSAAMVMEMQQSICQAAAQIQSELFPSTASANGNLQQSPVYQQTSHMMSALSTNEDMQMQCELFSSPPAVSGNETSTTTTQQVATPGTTMFQTSSSGDGEETGTQAKQIQNSVFQTMVQMQHSGDNQPQVNLFSSTKSMMSVQNSGTQQQGNGLFQQGNEMMSLQSGNFLQQSSHSQAQLFHPQNPIADAQNLSQETQGSLFHSPNPIVHSQTSTTSSEQMQPPMFHSQSTIAVLQGSSVPQDQQSTNIFLSQSPMNNLQTNTVAQEAFFAAPNSISPLQSTSNSEQQAAFQQQAPISHIQTPMLSQEQAQPPQQGLFQPQVALGSLPPNPMPQSQQGTMFQSQHSIVAMQSNSPSQEQQQQQQQQQQQQQQQQQSILFSNQNTMATMASPKQPPPNMIFNPNQNPMANQEQQNQSIFHQQSNMAPMNQEQQPMQFQSQSTVSSLQNPGPTQSESSQTPLFHSSPQIQLVQGSPSSQEQQVTLFLSPASMSALQTSINQQDMQQSPLYSPQNNMPGIQGATSSPQPQATLFHNTAGGTMNQLQNSPGSSQQTSGMFLFGIQNNCSQLLTSGPATLPDQLMAISQPGQPQNEGQPPVTTLLSQQMPENSPLASSINTNQNIEKIDLLVSLQNQGNNLTGSF

SEQ ID NO: 286 - DNA, 13285 bp, Homo sapiens
  source 1..13285: mol_type = genomic DNA
agattcctgtcagcggcggcggcggtggcggcgaccgtcagttttcgctgaggagaaacacgaaacggaccctttggctctcccccttccccttccccgtcctgaacccctctcctggtcaccgagaatcagtccccgtggagttccccctccacctcgccatcgtttcctcggtcctcggcccagtggaagtcactaccctcgaggaggaggcagcggcagccgccctcgcgtcgccgcccccggttcggtgcccgcggtcccggagaggaggtgccgccgccaccgccgctccccccctcccgctgccctcgggccgggctgggtcgagctgcgatgccctcggacttcatctcattgctcagcgcggacctagacctggaatcgcccaagtccctctactcgcgagattctctgaagttacacccatcacagaattttcatagagctggactattggaagaatctgtctatgatcttctcccaaaggagttacagttacctccatctagagaaacatctgtagcatcaatgagtcagacaagcggtggtgaggcaggctcgcctcctccagctgttgttgctgctgatgcttcttcagctccctcctcttcctccatgggcggtgcttgcagctcctttaccacctcttccagccctaccatttattctacctcagtcaccgacagcaaggctatgcaagtggagagctgctcctcagccgtgggggtaagtaacagaggggtaagtgaaaagcagttaaccagtaacacagttcagcagcatccatcaacaccgaagaggcacacagtcttgtacatctcaccaccacctgaggacttgctggataacagtcggatgtcctgccaggatgaggggtgtggattggaatctgagcagagctgcagtatgtggatggaggattccccctccaacttcagtaacatgagcaccagttcctacaatgataacactgaggtacctcgtaaatcacgaaaacgaaatccaaagcagaggccgggggtcaaacgacgagattgtgaagaatctaatatggatatatttgatgccgacagtgccaaagcacctcactatgtgctttctcagcttaccacggacaacaaaggcaactcaaaagcgggaaatggaacattggaaaaccaaaaaggaactggagtaaagaagagccctatgttgtgtggacaatatcctgttaaaagtgagggaaaggagctgaagatagttgtacaacctgagacacagcaccgagctcggtacctgactgagggcagccgtggctcagtgaaagatagaacacagcaaggctttcctacagtaaagctggaaggccataatgaacctgtagtgttgcaagtgtttgtgggcaacgactctggacgagtgaaaccacatggattttatcaggcctgcagagtaactggacgaaatacaactccttgcaaagaagtggacattgaaggcactactgttatagaagtcggccttgatcctagcaacaacatgacactggcggtggactgcgtagggatattgaaattgaggaatgctgatgtcgaagccagaataggaattgctggttccaagaagaaaagcactcgtgccagattggtttttcgagttaatatcatgaggaaagatggctccactttgacactgcaaacaccctcttctccaattttgtgtactcagccagcaggagtgccagaaatcttaaagaaaagcttgcatagctgttcagtgaaaggagaagaagaagtgtttttaatcggcaagaactttctgaaaggaactaaagttattttccaagaaaatgtttctgatgaaaactcttggaagtcagaagctgaaattgatatggaactatttcatcagaatcatcttattgtgaaggttcctccctatcatgaccaacatataactttgcctgtgtcagtgggaatatatgtagtgacaaatgctggaagatctcatgatgttcaaccattcacttacactccagacccagcagctggtgctttgaatgtaaatgtgaagaaggaaatatctagtccagcaagaccttgctcttttgaagaggccatgaaagcaatgaaaactactggatgtaatttagataaggtaaatattatccctaatgccctgatgactccactcataccaagcagtatgattaagagtgaagatgttactccaatggaagtaacagcagaaaaaagatcttccactatttttaagactacaaagtctgttggatcaactcagcaaacattagaaaacatctcaaacatagcaggaaatggctctttttcatcaccatcatcttcccacctaccttctgaaaatgaaaaacagcagcagattcagcccaaggcatacaacccagagaccctgacaactattcaaacccaggacatctcacagcctggtacttttccagcagtttctgcttctagtcagctgcccaacagcgatgcactattgcagcaggctacacagtttcagacaagagaaactcagtctagagagatattacagtcagatggtacagtggttaatttgtcacaactgactgaggcatcacaacaacagcagcagtcaccactacaagaacaagcacagactttacagcagcagatttcatcaaatatttttccatcaccaaatagtgtgagtcagcttcagaatactattcagcagctgcaagcagggagtttcacaggcagtactgctagtggcagcagtggaagtgttgacttggtccaacaagttttagaggcacagcagcagttatcttcagttttattttctgctccagatggtaatgagaatgttcaagagcagcttagtgcagatatttttcaacaagtcagtcaaattcagagtggtgtaagccctggaatgttttcctcaacagagccaacagtccataccagaccagataatttattacctggaagagctgaaagtgttcatccacagtctgaaaacacgttatctaatcaacagcagcagcagcagcagcaacagcaagtgatggaatcttcagccgcaatggtgatggagatgcaacagagtatctgccaggcagctgcccagattcagtcagagttattcccttcaactgcttcagcaaatggaaaccttcagcaatcgccagtttaccagcagacttctcacatgatgagtgcattgtctaccaatgaggatatgcaaatgcagtgtgaattgttttcttctcctcctgcagtttctggaaatgaaacttctacaactaccacacagcaggttgcaacccctggcactaccatgtttcagacatcaagttcaggagatggagaagaaactggaacacaagcaaaacagattcagaacagtgtctttcagaccatggtccaaatgcaacatagtggggacaatcaacctcaagttaaccttttttcatccacaaaaagtatgatgagtgttcagaatagtggtacccaacaacaaggtaatggtttattccagcaagggaatgagatgatgtcacttcaatctggaaattttttgcagcagtcttctcattcacaggcccaactttttcatcctcaaaatcctattgccgatgctcagaacctttcccaggaaactcaaggttctctctttcatagtccaaatcctattgtccacagtcagacttctacaacctcctctgaacaaatgcagcctccaatgtttcactctcaaagtaccattgctgtgttacagggctcttcagttcctcaagaccagcagtcaaccaacatatttctttcccagagtcccatgaataatcttcagactaacacagtagcccaagaagcattttttgcagcaccgaactcaatttctccacttcagtcaacatcaaacagtgaacaacaagctgctttccaacagcaagctccaatatcacacatccagactcctatgctttcccaagaacaggcacaacccccgcagcagggtttatttcagcctcaggtggccctgggctcccttccacctaatccaatgcctcaaagccaacaaggaaccatgttccagtcacagcactcaatagttgccatgcagagtaactctccatcccaggaacagcagcagcagcagcaacagcagcagcaacagcagcagcaacaacaacagagcattttattcagtaatcagaataccatggctacaatggcgtctccaaagcaaccaccaccaaacatgatattcaacccaaatcaaaatccaatggctaatcaggagcaacagaaccagtcaatttttcaccaacaaagtaacatggccccaatgaatcaagagcaacagcccatgcaatttcagagtcagtccacagtttcctcacttcagaacccaggtcctacccagtcggaatcatcacagacccccttgttccatagctctcctcagattcagttggtacaagggtcacctagttctcaagagcagcaagtaactctcttcttatctccagcatccatgtctgccttgcagaccagtataaatcaacaagatatgcaacagtctcctctttattcccctcagaacaacatgcctggaattcaaggagccacatcttcgcctcaaccacaggctactttatttcacaacacagcaggaggcacaatgaaccaactgcagaattctcctggctcatctcagcagacatcaggaatgttcttatttggcattcaaaataactgtagtcagcttttaacctctggaccagctacattgcctgatcagttgatggccataagtcagccaggccaaccacaaaacgagggccagccacctgtgacaacacttctttctcagcaaatgccagagaattctccactggcatcctctataaacaccaaccagaacatcgaaaagattgatttgcttgtttcattgcaaaaccaagggaacaacttgactggctccttttaactggatataaattccacgaagaaaatcctgattccaagatgtcctgagatcttgtggttccatgagaattattactttaaaaacaaaacaaaatataaaaaactgtgtttgagtaaactgatagattttactctgactgcaaaagagcacacctatgctgcttgttgcagtaactaaccaccaatgttaacatcttcatattttatattcctaataacagtgatgactgagaatctatttgagtttccagctggcagaattaattgttattattttcctaggcgcaatttccttaaacgtacagtttaaattcaaggctggaccactcagttattattgctattagaaaataatatatcatgtttacttttgttcttcattattttctttcctgcattgttttagtcaagtaatggcttttgaaaaagtaaagttcaataataactaaggctgtgatttttttcaatataaaaggcacagctgttggccaaagtgaaggaatcttttttcagttttattggagaaactgaagggtaacattctaacaagtaaactgtatgtgcagataaaagtactcttgatttaacacaaaggcagatgatacacttataaaactgggaacagctggaatgcttcttgattttattttttcagagagttgttagttctctgggtttctactaaggggtttagccataactgtgcatagaaaaataattatctgtaaaaaatgaaggggataatatatgataaattatgttctgatatcctcctacagtagtttaaattgacagaaaaatttgaatgttttcttcttaacccagtcttaggctggtattccctttttatatatatctatattacttttcacctctttttcactttactttagagaactattaatatactactggcttcatgaccctgtagcatctttggccactttaatctagggtgacctagcaatcctgcagcacagggcagagagtactgtcttaggaattattaggagttgattcctgagaaacaacacatttttccccatgaacggtgctgttctgaagtcttcaaatttttccctctaataggaaacagtataaattttaattaaaaaaaaaaggcaaactaaaatttcttgaaatatcacttctccctgatctgcagtgagtataaattcacttgtcacctcagtgctttacagtttgaagtggtcacttacctgatggttcccacaagccttaggctttacagggttgtatcattgacttaaaatgaagaattaacttgtgttacatctataaagagcaaaataacacactccagaacttggcagttgtagcattagttatacagttttgggtgttcttgccacccgtgggatgcctgcttctcactaccacctgtgtctggacacatgcttatgtctcattttccttttggcatgtggaaagctgtcaatgcagtgtaaggccaacgtgtgtgtggcttctatgtgttgagataatgttttggtatccttgtccgtttcatttattttttaagtgtacaaaaaataacctgttaattgttgaaggctacttttctgttctttttttttttttttttctatcctgtacatttagttgaactgtgcggaattgtggtgttggttttgtttacacagccagatttttccttctttttgttttgtgatgatcttcctttgttctttgaatgtgctcttttgtctttttctcttttttctcatgttttcttccctccacctccacccctttctttctttctctctctgattgagaggcattgaattacgttttcagtagtacaggcttcttgccgatatgaagggaacttttcagaaagagacctactctgggtcatttaattttgaatacagttttcaatcgttcaagttttggatggtttatatctaatgtgtgtttcatttttttggaaagctatattttgtatttaggaaatggtatactattttgctatttgtactgagtgagtacattggcataaatatagaaatttatatatatacatatatataaactattcttttttgccacacatttttgtggtaaatttgtgagtttgtctgatgttctaccacaacgtggcgtctgataacagtgagggggggtggggtttgttatgtctttattgagtatttaagtatcttttgaaacaaatgacctgttcatctgtggccattccatcaggcagttagttccttgatgtcagtagtgggctaaaggcagcttactgtgtgtttgctggagctttcactcagccaagtgttagagtcaggaaacccattgaggcaatggcgtcaaatggtgtttcacaagaatgagccattcagtctttgctcactatatatttaatattttattattgttgttattgttattattaattggctttctgtattctatgccttttatttataaagacactaagaaaacccatgtttgtaattttaataacatttttcccatcttgtaatatccagagctactttataaattctctgaaccaaaagtattttcctcagtgtatctcttctcccccagcccctattgggaaaaattacccagtatagttcaggttatgaggaggatcagccacacaatccagtgcttcagtttgaaaatgtaaaattctaaccctaaagtagggttggttgaaatttcagacaaagcaaacccagcaggtataaaaagtagtataaatacaaatctgtaagttatttttgaattttctgaacttttttctaagagattacataggagactaaagaaatctatctgttcaagttctaattaggatgattgttaatactgcactgtggatgaagtggcgactggcttgtgtgctgacttctgtggtttagcaagaggtttattgttatcaaatgctaattggcaatgccaagtcactgggaccaattttctgttttataatatctaagtttagaacagaatatatacctgaactgtagtggtttgatcggatggagacagaaaacccgatttttattctcataaattttgtggttatttatacaagggctgtgctatgctaccatattcttgttcaataataataggtttgttgttttttttacattgttaaatgttccttacccctaaaggtcaatgttaagtacaacattctgaaaatacaatttggctacgaagagtattcatcttctttgaagctcagtggttgatatttgtgctaataatgcaatttcctgattactgttacaagttatagctacatatgggagagactcagtgagccagcaaaggccatagaaacaacaatttattaaatgtatttatggcagaaggacctaaataaactgtgagccaccttttcttctttatattgttacatttaagtgttcttgctttcagcaactcacattaatgcttggagcttatctctttctctctctctctctctctctctgtgtgtgtgtgtgtatgtgtgtgtgtgtgtgtgtgtgtttccttattgtcattccattatatatccacaccaacatgggtgacgataattcaaagtcatattttgcctctaagcttgatcatgttacctttatgattaaagtatcatgttatttagccaatgcaaatctgttttaaaacaaatagtttaaaaaaagaacaagtttttaagggctttattatagaagaagtattaatgaaggactttccttcctccctccctttcctcccctccctgcctcccttcttcccttccatctccccctcctccctgccttctttgtttctccttcccttattcctccctccctcctttctcccttccttcctttcttccattcatccttccttgccttttatttttattttttgtaatatcacatgtgctgtagtttggaattttattctagtgcatttcttgctcatcagaacctcagctaatctacctaggaaaaatagtatcaaaggaaatgagaaagttgtatctgagtccctccagaactaagataattctttttgaccatttaagcctttataaatgcgttttgaccatttaagcctttataaatgcttgttttaggaaagtgaatctgttagatgcatcaacaaataatgaccaggacaaaacgatttaataattaaagtctcaaatcaccatggttatacattttcaccagaaatagtaatcttacaatttttcatttttctgatgaagatttctgttccaatatctgtttcctaatagattttttaaattaattagctttcctctgctttatgaccacaggttttatccctaaccgagacagctgtcttatatctgcatgccttagactgtgtggagggactccatgaagaaagaccataggttagaaaaataactcatagtatataccctagtaagtgggttagtagaatctcataacatgtattaaaaagaggttttcttctctgcttgtttgtgtcactagagcaaaattgtagagataatgctcataatgcagtaaatatcagaataatatctacaatatcatttgtggatggtcccaggtcccagtgctctagttactttacttctttttttttttttgagatggagtcttgctctgtctctcaggctagagcagtgtgcgatctcagctcactgcagcctccacctcccaggttcaagcgattctcctgcctcagcctcccaagtagccaggattacaggcaccctccactaggcccggctaattttttttgtatttttttagtagagatggggttttgccatgttggccaggctggtttcgaactcctaacctccagtgatccacctgcctcggcgtcccaaagtgctaggattacaggcatgagccaccacatccggcctaattacttctttaatccccatttatttttatgccattctagcctcatttattaataaaattatgtttttactttctctttcaggaaattttttaaattaatattttatatctagatctaatgctatggaaaagtgcctttttatcatttataatttcatttttcactatttccaaaaacacataaacaaatagtttcagtaggtcccagcttttactttttccatttaaaccttcttttctccatttcttccctttggcttaagaataaaagaaaaggtacattgctagaattgtttctttgggagagggtaaaagattacagaattagactgttcagcctttatataaactaaatttgtcttcatctcaaccagctaatggtaggtcttatctgaatactcatgagaattttagcatctgtgaaactccatgcaccagatgtgtgtaaatttcaggaagaaagtgttgaaagcattttctctgatgttaattagatggaaataaatcactaaaacatagtttaggtaaagcctgattatgccacttttttttaactagacagggcaaagttgtttatgttagtgtacttcttgtctatcctcagttaatttacctagacaaaaagtgtcaaaggaaatgagaaaaaggttatatctgactccctccagacctaagataattcctttgatcagatacagtcagatggagtgccttggtttttgttaattttgcctctattccagctccttaccacagcggtggtgcttaaagaaaggatcatcagcaacaggtcaggatagttctacctttgggatagggctgctttccccgtgctagtatttctgtgactgttagtggcactgaggactgcaaacttttatgcaatattcttaataccctattgatattatgcactttaatcattccaaagaagccaagaatgctgtatagtgatgattccttcctaatgaattcatcttaactatttagaatgttatgtcccttttcttttggatagccaacttggtataaatgttatatggatttttctaaaatgactatataggacttaagactttgaaatgtaatttacttataaggggaaataattatgctttagcacatcattttagaaacgtcacattttagaaacattcagcttgctaacctacatgtttgggaattcattaaaaccagttgtctatatattttgtgccatgtatataagaacattacaatatatctttttctacatatgtagtatgtgcaaccagtggttctcagagtatggttctcagcccaccagctagtatcagtatcacctgggaactagttagaaatgtaaattctttggccccatcccagacatactgagtcagaaactctggaatagggcccccgcaatctgttttcacaagccctccaggtgattctgatgcacactttaaagtttaggaaccactgggctaagactctgttgagatatagagtttttcttccactcagactgatatagttatacattgttcttcatgtaaattcagcttaacctggttatctataatcttttattggcaaaagttaattctcagtactgcctatagagatacagtgtattttatgtacatacacaattagtctaattcttgataattcagttaatttagtttggcattttcctaccacttactaaaaggtttacattaaatgactgatttaaatatataggtgcaatgttctatgtttattttaattgttatgacatttaagtagctaatataattgaccggtgctaaagtctcctgtttatccataaaatgggtacattatgggcagtgtaatacaagctttcttttcattgcctagtactttaccagcagaccacagttttgccctggctagaccaaccctcagaacaaaatcatcattccttgtatttatatttgtatctgagatagtaaacaagatggctggccaggtcaacatggcaccttaacttatttttttaataggtaaaacttcttcaaaagtagcttgctttgtataagaactaagctatcagtatagatatagctatccttggagcttatgtttcagacaagaattatttactaaaataaataataaacaagataatgcattatacaatttgggcatttctcgtttctcaagtgtatgcatcatggtaaatataaactaaccacaagataggtagattgattcatttcattttaatctccttgtgtaattcagtacctccataattgttctaatcttcttcccactgtttacaaattaccagttaattaactcgtgaaagaaaaattcacatatcagaataaaaataaatgtatactcactttataaaaatcaccactgctgtctttccttaatactagcagtggaaatgtaagtggcttactctacaaattttggtgctggcaaatacataggcaaactgttgggagctgctctagttacattcctcccttcttattccctttttctcttcctcactttattgcataacatattcctgtacccaaagcattctaccacagttctatttgactcccacttgtaataactcctttaaaaaattccatgtttaaccatatgaccctgcttgcttactcatattctccctccctctccccttcctttctctctcttccagaagtcatttgcctggtttgaaatattttgtagggattgcttattatattattttagctgatgaacctcaggacaacgtctacacacacacacatacatacacgcacacaaaatctcagctgttgaagagtgggcttggaatcagacttctgtgtccagtaaaaaactcctgcactgaagtcattgtgacttgagtagttacagactgattccagtgaacttgatctaatttcttttgatctaatgaatgtgtctgcttaccttgtttccttttaattgataagctccaagtagttgctaattttttgacaactttaaatgagtttcattcacttcttttacttaatgttttaagtatagtaccaataatttcattaacctgttctcaagtggtttagctaccattctgccatttttaatttttatttaattttatttgcttgagcacactgatcaaccactgaactgccttcttccattgtcctgcaatgatataagggttacatttttgtgtatatggctttcatagttgggatttcagagcactgataccagatattttcagtttgttctctgggggaatttcatttgcatctatgtttttagctatctgtgataacttgttaaatattaaaaagatattttgcttctattggaacatttgtatactcgcaactatatttctgtaaacagctgcagtcaaaaataaaacactgaaagttttca

SEQ ID NO: 287 - protein, 706 aa, Homo sapiens
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 288 - DNA, 3659 bp, Homo sapiens
  source 1..3659: mol_type = genomic DNA
acaagcgagctggtggttgaagctggttaaagaacagcctaggtattccagaagtgtttgaggatcccttccatgaaggaagagaggaaagtttttaagtaaacctcccactcccatgtgtcttcagctttcttttgcaaaggagaaaatccttgaagtttggtaaagaccgagttagtctatctctctttgcctatctcgagttgggctggggagaggaggagataggttcttttgtctttttctgtcttctcccttccccacttccttccctccagtccccactcactcacatgcacacactaaccttggagccgatgggattgagtgactggcacttgggaccacagagaaatgtcagagtgtttggttacagactcaaggaaacctctcattttagagtgctcatttggttttgagcaaaattttggactgtgaagcaaggcattggtgaagacaaaatggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgctgaagcatggagtgttgatgctttcgtctccagccccttctcagaatctacccaaaggatactgtaacactttacaatgttcatcccatgatgtagtgcctctttcatccactagtgcaaatcatagctgggggttgggggtggtgggggtcggggcctgggggactgggagccgcagcagctccccctcccccactgccataaaacattaagaaaatcatattgcttcttctcctatgtgtaaggtgaaccatgtcagcaaaaagcaaaatcattttatatgtcaaagcaggggagtatgcaaaagttctgacttgactttagtctgcaaaatgaggaatgtatatgttttgtgggaacagatgtttcttttgtatgtaaatgtgcattcttttaaaagacaagacttcagtatgttgtcaaagagagggctttaatttttttaaccaaaggtgaaggaatatatggcagagttgtaaatatataaatatatatatatataaaataaatatatataaacctaaaaaagatatattaaaaatataaaactgcgttaaaggctcgattttgtatctgcaggcagacacggatctgagaatctttattgagaaagagcacttaagagaatattttaagtattgcatctgtataagtaagaaaatattttgtctaaaatgcctcagtgtatttgtatttttttgcaagtgaaggtttacaatttacaaagtgtgtattaaaaaaaacaaaaagaacaaaaaaatctgcagaaggaaaaatgtgtaattttgttctagttttcagtttgtatatacccgtacaacgtgtcctcacggtgccttttttcacggaagttttcaatgatgggcgagcgtgcaccatccctttttgaagtgtaggcagacacagggacttgaagttgttactaactaaactctctttgggaatgtttgtctcatcccattctgcgtcatgcttgtgttataactactccggagacagggtttggctgtgtctaaactgcattaccgcgttgtaaaatatagctgtacaaatataagaataaaatgttgaaaagtcaaa

SEQ ID NO: 289 - protein, 320 aa, Homo sapiens
MELLSPPLRDVDLTAPDGSLCSFATTDDFYDDPCFDSPDLRFFEDLDPRLMHVGALLKPEEHSHFPAAVHPAPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAAFYAPGPLPPGRGGEHYSGDSDASSPRSNCSDGMMDYSGPPSGARRRNCYEGAYYNEAPSEPRPGKSAAVSSLDCLSSIVERISTESPAAPALLLADVPSESPPRRQEAAAPSEGESSGDPTQSPDAAPQCPAGANPNPIYQVL

SEQ ID NO: 290 - DNA, 1803 bp, Homo sapiens
  source 1..1803: mol_type = genomic DNA
aggggtgaggaagccctggggcgctgccgccgctttccttaaccacaaatcaggccggacaggagagggaggggtgggggacagtgggtgggcattcagactgccagcactttgctatctacagccggggctcccgagcggcagaaagttccggccactctctgccgcttgggttgggcgaagccaggaccgtgccgcgccaccgccaggatatggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcactcgcacttccccgcggcggtgcacccggccccgggcgcacgtgaggacgagcatgtgcgcgcgcccagcgggcaccaccaggcgggccgctgcctactgtgggcctgcaaggcgtgcaagcgcaagaccaccaacgccgaccgccgcaaggccgccaccatgcgcgagcggcgccgcctgagcaaagtaaatgaggcctttgagacactcaagcgctgcacgtcgagcaatccaaaccagcggttgcccaaggtggagatcctgcgcaacgccatccgctatatcgagggcctgcaggctctgctgcgcgaccaggacgccgcgccccctggcgccgcagccgccttctatgcgccgggcccgctgcccccgggccgcggcggcgagcactacagcggcgactccgacgcgtccagcccgcgctccaactgctccgacggcatgatggactacagcggccccccgagcggcgcccggcggcggaactgctacgaaggcgcctactacaacgaggcgcccagcgaacccaggcccgggaagagtgcggcggtgtcgagcctagactgcctgtccagcatcgtggagcgcatctccaccgagagccctgcggcgcccgccctcctgctggcggacgtgccttctgagtcgcctccgcgcaggcaagaggctgccgcccccagcgagggagagagcagcggcgaccccacccagtcaccggacgccgccccgcagtgccctgcgggtgcgaaccccaacccgatataccaggtgctctgaggggatggtggccgcccacccgcccgagggatggtgcccctagggtccctcgcgcccaaaagattgaacttaaatgcccccctcccaacagcgctttaaaagcgacctctcttgaggtaggagaggcgggagaactgaagtttccgcccccgccccacagggcaaggacacagcgcggttttttccacgcagcacccttctcggagacccattgcgatggccgctccgtgttcctcggtgggccagagctgaaccttgaggggctaggttcagctttctcgcgccctcccccatgggggtgagaccctcgcagacctaagccctgccccgggatgcaccggttatttgggggggcgtgagacccagtgcactccggtcccaaatgtagcaggtgtaaccgtaacccacccccaacccgtttcccggttcaggaccactttttgtaatacttttgtaatctattcctgtaaataagagttgctttgccagagcaggagcccctggggctgtatttatctctgaggcatggtgtgtggtgctacagggaatttgtacgtttataccgcaggcgggcgagccgcgggcgctcgctcaggtgatcaaaataaaggcgctaatttataccgcc

SEQ ID NO: 291 - protein, 255 aa, Homo sapiens
MDVMDGCQFSPSEYFYDGSCIPSPEGEFGDEFVPRVAAFGAHKAELQGSDEDEHVRAPTGHHQAGHCLMWACKACKRKSTTMDRRKAATMRERRRLKKVNQAFETLKRCTTTNPNQRLPKVEILRNAIRYIESLQELLREQVENYYSLPGQSCSEPTSPTSNCSDGMPECNSPVWSRKSSTFDSIYCPDVSNVYATDKNSLSSLDCLSNIVDRITSSEQPGLPLQDLASLSPVASTDSQPATPGASSSRLIYHVL

SEQ ID NO: 292 - DNA, 1540 bp, Homo sapiens
  source 1..1540: mol_type = genomic DNA
acccaggccaacaggcgtctgcccttgttaattaccggagcgacagactagggagctccgcccgggatttgcccatcggcggaggcgccaggctcccgtttctccccatccctctcgctgccgtccaggtgcaccgcctgcctctcagcaggatggacgtgatggatggctgccagttctcaccttctgagtacttctacgacggctcctgcataccgtcccccgagggtgaatttggggacgagtttgtgccgcgagtggctgccttcggagcgcacaaagcagagctgcagggctcagatgaggacgagcacgtgcgagcgcctaccggccaccaccaggctggtcactgcctcatgtgggcctgcaaagcctgcaagaggaagtccaccaccatggatcggcggaaggcagccactatgcgcgagcggaggcgcctgaagaaggtcaaccaggctttcgaaaccctcaagaggtgtaccacgaccaaccccaaccagaggctgcccaaggtggagatcctcaggaatgccatccgctacatcgagagcctgcaggagttgctgagagagcaggtggagaactactatagcctgccgggacagagctgctcggagcccaccagccccacctccaactgctctgatggcatgcccgaatgtaacagtcctgtctggtccagaaagagcagtacttttgacagcatctactgtcctgatgtatcaaatgtatatgccacagataaaaactccttatccagcttggattgcttatccaacatagtggaccggatcacctcctcagagcaacctgggttgcctctccaggatctggcttctctctctccagttgccagcaccgattcacagcctgcaactccaggggcttctagttccaggcttatctatcatgtgctatgaactaattttctggtctatatgacttcttccaggagggcctaatacacaggaagaagaaggcttcaaaaagtcccaaaccaagacaacatgtacataaagatttcttttcagttgtaaatttgtaaagattaccttgccactttataagaaagtgtatttaactaaaaagtcatcattgcaaataatactttcttcttctttattattctttgcttagatattaatacatagttccagtaatactatttctgatagggggccattgattgagggtagcttgttgcaatgcttaacttatatatacatatatatatattataaatattgctcatcaaaatgtctctggtgtttagagctttatttttttctttaaaacattaaaacagctgagaatcagttaaatggaattttaaatatatttaactatttcttttctctttaatcctttagttatattgtattaaataaaaatataatactgcctaatgtatatattttgatcttttcttgtaagaaatgtatcttttaaatgtaagcacaaaatagtactttgtggatcatttcaagatataagaaattttggaaattccaccataaataaaattttttactacaagaaa

SEQ ID NO: 293 - protein, 224 aa, Homo sapiens
MELYETSPYFYQEPRFYDGENYLPVHLQGFEPPGYERTELTLSPEAPGPLEDKGLGTPEHCPGQCLPWACKVCKRKSVSVDRRRAATLREKRRLKKVNEAFEALKRSTLLNPNQRLPKVEILRSAIQYIERLQALLSSLNQEERDLRYRGGGGPQPGVPSECSSHSASCSPEWGSALEFSANPGDHLLTADPTDAHNLHSLTSIVDSITVEDVSVAFPDETMPN

SEQ ID NO: 294 - DNA, 1507 bp, Homo sapiens
  source 1..1507: mol_type = genomic DNA
gggctgctggagcttgggggctggtggcaggaacaagccttttccgaccccatggagctgtatgagacatccccctacttctaccaggaaccccgcttctatgatggggaaaactacctgcctgtccacctccagggcttcgaaccaccaggctacgagcggacggagctcaccctgagccccgaggccccagggccccttgaggacaaggggctggggacccccgagcactgtccaggccagtgcctgccgtgggcgtgtaaggtgtgtaagaggaagtcggtgtccgtggaccggcggcgggcggccacactgagggagaagcgcaggctcaagaaggtgaatgaggccttcgaggccctgaagagaagcaccctgctcaaccccaaccagcggctgcccaaggtggagatcctgcgcagtgccatccagtacatcgagcgcctccaggccctgctcagctccctcaaccaggaggagcgtgacctccgctaccggggcgggggcgggccccagccaggggtgcccagcgaatgcagctctcacagcgcctcctgcagtccagagtggggcagtgcactggagttcagcgccaacccaggggatcatctgctcacggctgaccctacagatgcccacaacctgcactccctcacctccatcgtggacagcatcacagtggaagatgtgtctgtggccttcccagatgaaaccatgcccaactgagattgtcttccaagccgggcatccttgcgagccccccaagctggccacagatgccactacttctgtagcaggggcctcctaagccaggctgccctgatgctaggaagccagctctggggtgccataggccagactatccccttcctcatccatgtaaggttaacccaccccccagcaagggactggacgccctcattcagctgcctccttagaggagagggcatcccctttccagggaggtaaagcaggggaccagagcgccccctcgtgtatgccccagctcagggggcaaactcaggagcttcctttttatcataacgcggcctctaattccaccccccaagtgaaacggtttgagagacgcagtgccctgacctggacaagctgtgcacgtctcctgttctggtctcttcccgatgccagtggctgggctgggcctgccctgaattgagagagaagaaggggagaggaacagccctctgttcccaagtccctggggggccaaacttttgcagtgaatattgggaaccttccagtggttttatgttttgttttgtttcgtgtgttgtttgtaaagctgccatccgaccaaggtctcctgtgctgaagttgccggggacaggcagggaaaaggggttggggcctcttgggggtgatttcttttgttaacaaagcattgtgtggttttgccattgttttgtattttttttttttttttttttttttgctaacttatttggatttccttttttaaaaaatgaataaagactggttgccagaa

SEQ ID NO: 295 - protein, 271 aa, Homo sapiens
MLQACKMEGFPLVPPQPSEDLVPYDTDLYQRQTHEYYPYLSSDGESHSDHYWDFHPHHVHSEFESFAENNFTELQSVQPPQLQQLYRHMELEQMHVLDTPMVPPHPSLGHQVSYLPRMCLQYPSLSPAQPSSDEEEGERQSPPLEVSDGEADGLEPGPGLLPGETGSKKKIRLYQFLLDLLRSGDMKDSIWWVDKDKGTFQFSSKHKEALAHRWGIQKGNRKKMTYQKMARALRNYGKTGEVKKVKKKLTYQFSGEVLGRGGLAERRHPPH

SEQ ID NO: 296 - DNA, 1377 bp, Homo sapiens
  source 1..1377: mol_type = genomic DNA
aaaatcaggaacttgtgctggccctgcaatgtcaagggagggggctcacccagggctcctgtagctcagggggcaggcctgagccctgcacccgccccacgaccgtccagcccctgacggggcaccccatcctgaggggctctgcattggcccccaccgaggcaggggatctgaccgactcggagcccggctggatgttacaggcgtgcaaaatggaagggtttcccctcgtcccccctcagccatcagaagacctggtgccctatgacacggatctataccaacgccaaacgcacgagtattacccctatctcagcagtgatggggagagccatagcgaccattactgggacttccacccccaccacgtgcacagcgagttcgagagcttcgccgagaacaacttcacggagctccagagcgtgcagcccccgcagctgcagcagctctaccgccacatggagctggagcagatgcacgtcctcgatacccccatggtgccaccccatcccagtcttggccaccaggtctcctacctgccccggatgtgcctccagtacccatccctgtccccagcccagcccagctcagatgaggaggagggcgagcggcagagccccccactggaggtgtctgacggcgaggcggatggcctggagcccgggcctgggctcctgcctggggagacaggcagcaagaagaagatccgcctgtaccagttcctgttggacctgctccgcagcggcgacatgaaggacagcatctggtgggtggacaaggacaagggcaccttccagttctcgtccaagcacaaggaggcgctggcgcaccgctggggcatccagaagggcaaccgcaagaagatgacctaccagaagatggcgcgcgcgctgcgcaactacggcaagacgggcgaggtcaagaaggtgaagaagaagctcacctaccagttcagcggcgaagtgctgggccgcgggggcctggccgagcggcgccacccgccccactgagcccgcagcccccgccgggccccgccaggcctccccgctggccatagcattaagccctcgcccggcccggacacagggaggacgctcccggggcccagaggcaggactgtggcgggccgggcctcgcctcacccgccccctccccccactccaggccccctccacatcccgcttcgcctccctccaggactccaccccggctcccggacgccagctgggcgtcagaccccaccggggcaaccttgcagaggacgacccggggtactgccttgggagtctcaagtccgtatgtaaatcagatctcccctctcacccctcccacccattaacctcctcccaaaaaacaagtaaagttattctcaatcca

SEQ ID NO: 297 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 298 - DNA, 3820 bp, Homo sapiens
  source 1..3820: mol_type = genomic DNA
aaacagccagtgcagaggagaggaacgtgtgtccagtgtcccgatccctgcggagctagtagctgagagctctgtgccctgggcaccttgcagccctgcacctgcctgccacttccccaccgaggccatgggcccaggagttctgctgctcctgctggtggccacagcttggcatggtcagggaatcccagtgatagagcccagtgtccctgagctggtcgtgaagccaggagcaacggtgaccttgcgatgtgtgggcaatggcagcgtggaatgggatggccccccatcacctcactggaccctgtactctgatggctccagcagcatcctcagcaccaacaacgctaccttccaaaacacggggacctatcgctgcactgagcctggagaccccctgggaggcagcgccgccatccacctctatgtcaaagaccctgcccggccctggaacgtgctagcacaggaggtggtcgtgttcgaggaccaggacgcactactgccctgtctgctcacagacccggtgctggaagcaggcgtctcgctggtgcgtgtgcgtggccggcccctcatgcgccacaccaactactccttctcgccctggcatggcttcaccatccacagggccaagttcattcagagccaggactatcaatgcagtgccctgatgggtggcaggaaggtgatgtccatcagcatccggctgaaagtgcagaaagtcatcccagggcccccagccttgacactggtgcctgcagagctggtgcggattcgaggggaggctgcccagatcgtgtgctcagccagcagcgttgatgttaactttgatgtcttcctccaacacaacaacaccaagctcgcaatccctcaacaatctgactttcataataaccgttaccaaaaagtcctgaccctcaacctcgatcaagtagatttccaacatgccggcaactactcctgcgtggccagcaacgtgcagggcaagcactccacctccatgttcttccgggtggtagagagtgcctacttgaacttgagctctgagcagaacctcatccaggaggtgaccgtgggggaggggctcaacctcaaagtcatggtggaggcctacccaggcctgcaaggttttaactggacctacctgggacccttttctgaccaccagcctgagcccaagcttgctaatgctaccaccaaggacacatacaggcacaccttcaccctctctctgccccgcctgaagccctctgaggctggccgctactccttcctggccagaaacccaggaggctggagagctctgacgtttgagctcacccttcgataccccccagaggtaagcgtcatatggacattcatcaacggctctggcacccttttgtgtgctgcctctgggtacccccagcccaacgtgacatggctgcagtgcagtggccacactgataggtgtgatgaggcccaagtgctgcaggtctgggatgacccataccctgaggtcctgagccaggagcccttccacaaggtgacggtgcagagcctgctgactgttgagaccttagagcacaaccaaacctacgagtgcagggcccacaacagcgtggggagtggctcctgggccttcatacccatctctgcaggagcccacacgcatcccccggatgagttcctcttcacaccagtggtggtcgcctgcatgtccatcatggccttgctgctgctgctgctcctgctgctattgtacaagtataagcagaagcccaagtaccaggtccgctggaagatcatcgagagctatgagggcaacagttatactttcatcgaccccacgcagctgccttacaacgagaagtgggagttcccccggaacaacctgcagtttggtaagaccctcggagctggagcctttgggaaggtggtggaggccacggcctttggtctgggcaaggaggatgctgtcctgaaggtggctgtgaagatgctgaagtccacggcccatgctgatgagaaggaggccctcatgtccgagctgaagatcatgagccacctgggccagcacgagaacatcgtcaaccttctgggagcctgtacccatggaggccctgtactggtcatcacggagtactgttgctatggcgacctgctcaactttctgcgaaggaaggctgaggccatgctgggacccagcctgagccccggccaggaccccgagggaggcgtcgactataagaacatccacctcgagaagaaatatgtccgcagggacagtggcttctccagccagggtgtggacacctatgtggagatgaggcctgtctccacttcttcaaatgactccttctctgagcaagacctggacaaggaggatggacggcccctggagctccgggacctgcttcacttctccagccaagtagcccagggcatggccttcctcgcttccaagaattgcatccaccgggacgtggcagcgcgtaacgtgctgttgaccaatggtcatgtggccaagattggggacttcgggctggctagggacatcatgaatgactccaactacattgtcaagggcaatgcccgcctgcctgtgaagtggatggccccagagagcatctttgactgtgtctacacggttcagagcgacgtctggtcctatggcatcctcctctgggagatcttctcacttgggctgaatccctaccctggcatcctggtgaacagcaagttctataaactggtgaaggatggataccaaatggcccagcctgcatttgccccaaagaatatatacagcatcatgcaggcctgctgggccttggagcccacccacagacccaccttccagcagatctgctccttccttcaggagcaggcccaagaggacaggagagagcgggactataccaatctgccgagcagcagcagaagcggtggcagcggcagcagcagcagtgagctggaggaggagagctctagtgagcacctgacctgctgcgagcaaggggatatcgcccagcccttgctgcagcccaacaactatcagttctgctgaggagttgacgacagggagtaccactctcccctcccacaaacttcaactcctccatggatggggcgacacggggagaacatacaaactctgccttcggtcatttcactcaacagctcggcccagctctgaaacttgggaaggtgagggattcaggggaggtcagaggatcccacttcctgagcatgggccatcactgccagtcaggggctgggggctgagccctcacccccccctcccctactgttctcatggtgttggcctcgtgtttgctatgccaactagtagaaccttctttcctaatccccttatcttcatggaaatggactgactttatgcctatgaagtccccaggagctacactgatactgagaaaaccaggctctttggggctagacagactggcagagagtgagatctccctctctgagaggagcagcagatgctcacagaccacactcagctcaggccccttggagcaggatggctcctctaagaatctcacaggacctcttagtctctgccctatacgccgccttcactccacagcctcacccctcccacccccatactggtactgctgtaatgagccaagtggcagctaaaagttgggggtgttctgcccagtcccgtcattctgggctagaaggcaggggaccttggcatgtggctggccacaccaagcaggaagcacaaactcccccaagctgactcatcctaactaacagtcacgccgtgggatgtctctgtccacattaaactaacagcattaatgca

SEQ ID NO: 299 - protein, 304 aa, Homo sapiens
MITSETGKALIQTCLNSPDSPPRSDPTTDQRMSATGFEETDLTYQVSESDSSGETKDTLKPAFTVTNLPGTTSTIQTAPSTSTTMQVSSGPSFPITNYLAPVSASVSPSAVSSANGTVLKSTGSGPVSSGGLMQLPTSFTLMPGGAVAQQVPVQAIQVHQAPQQASPSRDSSTDLTQTSSSGTVTLPATIMTSSVPTTVGGHMMYPSPHAVMYAPTSGLGDGSLTVLNAFSQAPSTMQVSHSQVQEPGGVPQVFLTASSGTVQIPVSAVQLHQMAVIGQQAGSSSNLTELQVVNLDTAHSTKSE

SEQ ID NO: 300 - DNA, 3386 bp, Homo sapiens
  source 1..3386: mol_type = genomic DNA
gcagaggtgggagtgaccggcgccagagaggaagagggcccttgctgagtgaaggggcctatgagctgtccacgctgacagggacacaggtgctgttgctggtggccagtgagacaggccatgtgtatacctttgccacccgaaaactgcagcccatgatcaccagtgagaccggcaaggcactgattcagacctgcctcaactcgccagactctccaccccgttcagaccccacaacagaccagagaatgagtgccactggctttgaagagacagatctcacctaccaggtgtcggagtctgacagcagtggggagaccaaggacacactgaagccggcgttcacagtcaccaacctgccgggtacaacctccaccatccaaacagcacctagcacctctaccaccatgcaagtcagcagcggcccctcctttcccatcaccaactacctggcaccagtgtctgctagtgtcagccccagtgctgtcagcagtgccaatgggactgtgctgaagagtacaggcagcggccctgtctcctctgggggccttatgcagctgcctaccagcttcaccctcatgcctggtggggcagtggcccagcaggtcccagtgcaggccattcaagtgcaccaggccccacagcaagcgtctccctcccgtgacagcagcacagacctcacgcagacctcctccagcgggacagtgacgctgcccgccaccatcatgacgtcatccgtgcccacaactgtgggtggccacatgatgtaccctagcccgcatgcggtgatgtatgcccccacctcgggcctgggtgatggcagcctcaccgtgctgaatgccttctcccaggcaccatccaccatgcaggtgtcacacagccaggtccaggagccaggtggcgtcccccaggtgttcctgacagcatcatctgggacagtgcagatccctgtttcagcagttcagctccaccagatggctgtgatagggcagcaggccgggagcagcagcaacctcaccgagctacaggtggtgaacctggacaccgcccacagcaccaagagtgaatgatccgcccgccgccctggacagatggcccaagggatggcaccacttatttattgttgccttttcacgttttctttacacacacgttgacgggccgcaggagggaggcggggaggaggaacgggcagccacaggactgagccctctcactccagccaaagaaatgggcctgcctgcctccacccgtcctccctcagcctccccttcttcccgccccacctcccatttctgttgctggaggggctgtcctccttcctgggaccccctcgccagcttggctcgatgtttgccatgagtattagcttacccaatgggaccgtgccccacctccccacacacaggccttctgtggggctgggcaccgtgtcctcctctgaggaagcagttggggccctcttgccagcctccttgctgaccccaggtcagccctgtgtctgtcacaggctgggtcaaaagagccctggctctgcccctcagggggccagctggggagatgggggcttcttcctcacactgctgtcctctcccccttcagctcctgagtagctgggcctgtgcactgggcaggttcctggggccgcctgccctgccttgccgctccccttggacctccaggggctcctgggttggagggaaccaccagcgttcccttctcccccttgtcttcccccctctcctcccagctgctttacttaaagttgattttgaactttttatttgaggagacgaagtgaaaacaaatctataaatatatatttttaaaatatttaacttttttttatggcgtttttctcgtccccctccctgcccaaactccccttccctggggagccctcaggctccccagaactggctgggcccctggggacagagccaccccatgagctcggggtccaccagtgtgtgggggagattctgggtttgcccagtcctgggttgtttccaggagaaagccgggggaggggccctcaggccattccccaacggggtggggagggtgacccacagctctgggcctctttttgccctttagggctgttgctagggagagggaagagggagaccaaatgtcggggttggggtgggagggcgtcaggcagaggcaactgacttcatttgtgccacacgcatgggcattgcagccttgcgctgtcccaggcatgcagctgcctggggcccaagttgcagtgagcagggtggggtctgggagggggtgagaggcaggaatgggggtcagaagaagtgggagcagcttcttgggctgagtgcagccaaaggggagccagaaatgggcagttctcccagggagtgagcagctactgtaacttttttaaattaagacaaaaagccttgaagaaaatgactttatttttctaagtgtaacctcagtatttatgtaatttgtacaggggccatgccccacccccctcctccccctttggggtagaccttgagggtgggccagcataggggggagggtcttttaccctgtgtcagagcctaccttcaccacctatatccagaaggggagctttttcagaaacagggcagcagtggggtgaaattttcttaacccctaagactgccttcagtaggaacaagctggcttctgtgattaggtgaagggatgggggaagattttatgcacagcctagttatcaaggggatgatttgccgacatgtttgagaaccccctaacctctaaccctcattgctgtcttgccccagtttggggtgccaagatggaagtcacctttctgggctttctcctggagatagctggggcttatgggtggctttcaaggctggggcatggcaaatcaggggccagagagcaggggagcttgggactcaggtctgtaactgcccagccccttttctctgctcttgtttcactccaccatcactcactcactccccactcccccacccatggggaggagacctttgatgaattcttcctctccttcccacaaaagacagacccagtgagtgaatcaggcaaagtgcttataatgtgtgttgtgtgagcgtggccttgggaggacatgcgtgtgtcagggatgagttgaggtgatatttttatgtgcagcgacccttggtgtttcccttcctcggtggctctggggtatgtgtgtgtgggtgtgtgcgcctgagtgagtgtgtgtgcttgaatgtgagtgtgtatgtcagtggtttctacttcccctgggatgctgacccaggaatagtggacatggtcacagtcctatgtacagagctttcttttgtattaaaaaaaaatactctttcaataaatgtatcatttttgtgcacaga

SEQ ID NO: 301 - protein, 1586 aa, Homo sapiens
METSASATASEKQEAKSGILEAAGFPDPGKKASPLVVAAAAAAAVAAQGVPQHLLPPFHAPLPIDMRHQEGRYHYEPHSVHGVHGPPALSGSPVISDISLIRLSPHPAGPGESPFNAPHPYVNPHMEHYLRSVHSSPTLSMISAARGLSPADVAQEHLKERGLFGLPAPGTTPSDYYHQMTLVAGHPAPYGDLLMQSGGAASAPHLHDYLNPVDVSRFSSPRVTPRLSRKRALSISPLSDASLDLQRMIRTSPNSLVAYINNSRSSSAASGSYGHLSAGALSPAFTFPHPINPVAYQQILSQQRGLGSAFGHTPPLIQPSPTFLAQQPMALTSINATPTQLSSSSNCLSDTNQNKQSSESAVSSTVNPVAIHKRSKVKTEPEGLRPASPLALTQGQVSGHGSCGCALPLSQEQLADLKEDLDRDDCKQEAEVVIYETNCHWEDCTKEYDTQEQLVHHINNEHIHGEKKEFVCRWQACTREQKPFKAQYMLVVHMRRHTGEKPHKCTFEGCSKAYSRLENLKTHLRSHTGEKPYVCEHEGCNKAFSNASDRAKHQNRTHSNEKPYICKIPGCTKRYTDPSSLRKHVKTVHGPDAHVTKKQRNDVHLRTPLLKENGDSEAGTEPGGPESTEASSTSQAVEDCLHVRAIKTESSGLCQSSPGAQSSCSSEPSPLGSAPNNDSGVEMPGTGPGSLGDLTALDDTPPGADTSALAAPSAGGLQLRKHMTTMHRFEQLKKEKLKSLKDSCSWAGPTPHTRNTKLPPLPGSGSILENFSGSGGGGPAGLLPNPRLSELSASEVTMLSQLQERRDSSTSTVSSAYTVSRRSSGISPYFSSRRSSEASPLGAGRPHNASSADSYDPISTDASRRSSEASQCSGGSGLLNLTPAQQYSLRAKYAAATGGPPPTPLPGLERMSLRTRLALLDAPERTLPAGCPRPLGPRRGSDGPTYGHGHAGAAPAFPHEAPGGGARRASDPVRRPDALSLPRVQRFHSTHNVNPGPLPPCADRRGLRLQSHPSTDGGLARGAYSPRPPSISENVAMEAVAAGVDGAGPEADLGLPEDDLVLPDDVVQYIKAHASGALDEGTGQVYPTESTGFSDNPRLPSPGLHGQRRMVAADSNVGPSAPMLGGCQLGFGAPSSLNKNNMPVQWNEVSSGTVDALASQVKPPPFPQGNLAVVQQKPAFGQYPGYSPQGLQASPGGLDSTQPHLQPRSGAPSQGIPRVNYMQQLRQPVAGSQCPGMTTTMSPHACYGQVHPQLSPSTISGALNQFPQSCSNMPAKPGHLGHPQQTEVAPDPTTMGNRHRELGVPDSALAGVPPPHPVQSYPQQSHHLAASMSQEGYHQVPSLLPARQPGFMEPQTGPMGVATAGFGLVQPRPPLEPSPTGRHRGVRAVQQQLAYARATGHAMAAMPSSQETAEAVPKGAMGNMGSVPPQPPPQDAGGAPDHSMLYYYGQIHMYEQDGGLENLGSCQVMRSQPPQPQACQDSIQPQPLPSPGVNQVSSTVDSQLLEAPQIDFDAIMDDGDHSSLFSGALSPSLLHSLSQNSSRLTTPRNSLTLPSIPAGISNMAVGDMSSMLTSLAEESKFLNMMT

SEQ ID NO: 302 - DNA, 7187 bp, Homo sapiens
  source 1..7187: mol_type = genomic DNA
gctgatggattgcagaagtgccggcgcttgccagccgaggcagcacggctccgcggactttttttcaaactcccatcaatgagacttcgaggaggagcgggcggcggcggcggctgcgactgcgaacgcggaggaaggccaggagccgcaggaggagccggaggaaagagcttgggccgcgcggcgcgccgcagcctcggggagccgcctgctcgccggcggtaggggctgcgcggcgcccgcccgcctctcggtcccctctcttgcctggcccgccccgccccggctggctggagccccggcacaaggcagccagccgagggtcgccgcgccagccaaggtgggatgggggcccacagccaccgcccggcgcccgagaggccacctgcgtgctagaggcaaacttttgtctctctcggattgccacccaggacgatgagcggctgagatggagacgtctgcctcagccactgcctccgagaagcaagaagccaaaagtgggatcctggaggccgctggcttccccgacccgggtaaaaaggcctctcctttggtggtggctgcagcggcagcagcagcggtagctgcccaaggagtgccgcagcatctcttgccaccattccatgcgcccctaccgattgacatgcgacaccaggaaggaaggtaccattacgagcctcattctgtccacggtgtgcacgggccccctgccctcagcggcagccctgtcatctctgacatctccttgatccggctttccccgcacccggctggccctggggagtcccccttcaacgccccccacccgtacgtgaacccccacatggagcactacctccgttctgtgcacagcagccccacgctctccatgatctctgcagccaggggcctcagccccgctgatgtggcccaggagcaccttaaggagaggggactgtttggccttcctgctccaggcaccaccccctcagactattaccaccagatgaccctcgtggcaggccaccccgcgccctacggggacctgctgatgcagagcgggggcgctgccagcgcaccccatctccacgactacctcaaccccgtggacgtgtcccgtttctccagcccgcgggtgacgccccgcctgagccgcaagcgggcgctgtccatctccccactctcagacgccagcctggacctgcagcggatgatccgcacctcacccaactcgctagtggcctacatcaacaactcccgaagcagctcggcggccagcggttcctacgggcatctgtcagcgggtgccctcagcccagccttcaccttcccccaccccatcaaccccgtggcctaccagcagattctgagccagcagaggggtctggggtcagcctttggacacacaccacccctgatccagccctcacccaccttcctggcccagcagcccatggccctcacctccatcaatgccacgcccacccagctcagcagcagcagcaactgtctgagtgacaccaaccagaacaagcagagcagtgagtcggccgtcagcagcaccgtcaaccctgtcgccattcacaagcgcagcaaggtcaagaccgagcctgagggcctgcggccggcctcccctctggcgctgacgcagggccaggtgtctggacacggctcatgtgggtgtgcccttcccctctcccaggagcagctggctgacctcaaggaagatctggacagggatgactgtaagcaggaggctgaggtggtcatctatgagaccaactgccactgggaagactgcaccaaggagtacgacacccaggagcagctggtgcatcacatcaacaacgagcacatccacggggagaagaaggagtttgtgtgccgctggcaggcctgcacgcgggagcagaagcccttcaaggcgcagtacatgctggtggtgcacatgcggcgacacacgggcgagaagccccacaagtgcacgttcgagggctgctcgaaggcctactcccgcctggagaacctgaagacacacctgcggtcccacaccggggagaagccatatgtgtgtgagcacgagggctgcaacaaagccttctccaacgcctcggaccgcgccaagcaccagaatcgcacccactccaacgagaaaccctacatctgcaagatcccaggctgcaccaagagatacacagaccccagctctctccggaagcatgtgaaaacggtccacggcccagatgcccacgtcaccaagaagcagcgcaatgacgtgcacctccgcacaccgctgctcaaagagaatggggacagtgaggccggcacggagcctggcggcccagagagcaccgaggccagcagcaccagccaggccgtggaggactgcctgcacgtcagagccatcaagaccgagagctccgggctgtgtcagtccagccccggggcccagtcgtcctgcagcagcgagccctctcctctgggcagtgcccccaacaatgacagtggcgtggagatgccggggacggggcccgggagcctgggagacctgacggcactggatgacacacccccaggggccgacacctcagccctggctgccccctccgctggtggcctccagctgcgcaaacacatgaccaccatgcaccggttcgagcagctcaagaaggagaagctcaagtcactcaaggattcctgctcatgggccgggccgactccacacacgcggaacaccaagctgcctcccctcccgggaagtggctccatcctggaaaacttcagtggcagtgggggcggcgggcccgcggggctgctgccgaacccgcggctgtcggagctgtccgcgagcgaggtgaccatgctgagccagctgcaggagcgccgcgacagctccaccagcacggtcagctcggcctacaccgtgagccgccgctcctccggcatctccccctacttctccagccgccgctccagcgaggcctcgcccctgggcgccggccgcccgcacaacgcgagctccgctgactcctacgaccccatctccacggacgcgtcgcggcgctcgagcgaggccagccagtgcagcggcggctccgggctgctcaacctcacgccggcgcagcagtacagcctgcgggccaagtacgcggcagccactggcggccccccgcccactccgctgccgggcctggagcgcatgagcctgcggaccaggctggcgctgctggacgcgcccgagcgcacgctgcccgccggctgcccacgcccactggggccgcggcgtggcagcgacgggccgacctatggccacggccacgcgggggctgcgcccgccttcccccacgaggctccaggcggcggagccaggcgggccagcgaccctgtgcggcggcccgatgccctgtccctgccgcgggtgcagcgcttccacagcacccacaacgtgaaccccggcccgctgccgccctgtgccgacaggcgaggcctccgcctgcagagccacccgagcaccgacggcggcctggcccgcggcgcctactcgccccggccgcctagcatcagcgagaacgtggcgatggaggccgtggcggcaggagtggacggcgcggggcccgaggccgacctggggctgccggaggacgacctggtgcttccagacgacgtggtgcagtacatcaaggcgcacgccagtggcgctctggacgagggcaccgggcaggtgtatcccacggaaagcactggcttctctgacaaccccagactacccagcccggggctgcacggccagcgcaggatggtggctgcggactccaacgtgggcccctccgcccctatgctgggaggatgccagttaggctttggggcgccctccagcctgaacaaaaataacatgcctgtgcagtggaatgaggtgagctccggcaccgtagacgccctggccagccaggtgaagcctccaccctttcctcagggcaacctggcggtggtgcagcagaagcctgcctttggccagtacccgggctacagtccgcaaggcctacaggctagccctgggggcctggacagcacgcagccacacctgcagccccgcagcggagccccctcccagggcatccccagggtaaactacatgcagcagctgcgacagccagtggcaggcagccagtgtcctggcatgactaccactatgagcccccatgcctgctatggccaagtccacccccagctgagccccagcaccatcagtggggccctcaaccagttcccccaatcctgcagcaacatgccagccaagccagggcatctggggcaccctcagcagacagaagtggcacctgaccccaccacgatgggcaatcgccacagggaacttggggtccccgattcagccctggctggagtgccaccacctcacccagtccagagctacccacagcagagccatcacctggcagcctccatgagccaggagggctaccaccaggtccccagccttctgcctgcccgccagcctggcttcatggagccccaaacaggcccgatgggggtggctacagcaggctttggcctagtgcagccccggcctcccctcgagcccagccccactggccgccaccgtggggtacgtgctgtgcagcagcagctggcctacgccagggccacaggccatgccatggctgccatgccgtccagtcaggaaacagcagaggctgtgcccaagggagcgatgggcaacatggggtcggtgcctccccagccgcctccgcaggacgcaggtggggccccggaccacagcatgctctactactacggccagatccacatgtacgaacaggatggaggcctggagaacctcgggagctgccaggtcatgcggtcccagccaccacagccacaggcctgtcaggacagcatccagccccagcccttgccctcaccaggggtcaaccaggtgtccagcactgtggactcccagctcctggaggccccccagattgacttcgatgccatcatggatgatggcgatcactcgagtttgttctcgggtgctctgagccccagcctcctccacagcctctcccagaactcctcccgcctcaccaccccccgaaactccttgaccctgccctccatccccgcaggcatcagcaacatggctgtcggggacatgagctccatgctcaccagcctcgccgaggagagcaagttcctgaacatgatgacctagaggcccgagcgcctggtgctgagtgcacccggaggggtcatcgctgcccagagcctggggattccagctgtcttgtctttttccaaaaaagtgttaaataggcttgaggggttgttgcgcaatggccgcttcagatgacagatgttgtaagagaaggtttatgggcatcctctctggtcttttggattattcctcagaacaatgaaaaaagtctccataggacaggaaggaatgcaaaactcatttacacagtgctttccagcctttggtgcttacaggaccgcgctgttccggcttcttcacggctgacattcggctaacgagggattactttggccaaaacctttcaaaggatatgcagaaagatggtagggagcatttgggtttgaatctgaatgctatactggatactctgctccggaaagatgagctttttattctactacttggaaggaaaaggaattcctggtccacctgaattcctctatgaagcctaactcttgaggtctctaacataccttgtcatagaggaaaagcacagattatacctggatgattcaggagcacattctgattccaggtttggtagagctggctcttctactccgtaaagccgagtctgggactggcagcccatccaagtgtatatgaatgaataaagcatccaagtatatatgaatgaataaagtatgtaagtatcaccagaaaaaggaaagaaaaaatgtactccttggggcaagcccagaagctgccctggcctctccagaccgtgtttacagtgtttgcatgtagaatgtagcccttcctgaaaagaagacttgtttctaaatacctcggggctgctggagccgctgtgggttagggatggactgaggcctcgaggagtgagggtgcacccggggcccagcctcaggctgccctagggatctctcagtaggaagaggaagttgcgtgtttacccaatcctgtttctccaatgcaacgtccacccactttaccaccaaaaactccagggcctgacggcagcccggtcccccagcactcaccagcagcccagtgttctccaccaagccacagtgtgcatgcctggtatcctccggattcccttccttctgcccgctgagtcactgggcagagaatgatgacatgtgtaggtggtgtggttgggggtggaaaggggaaggggttgatcctcaggactctgagggagcatcgttgaattttcctgttcagtgtgaccaagacccacctggaaatggaatttggaactggcttcaggagacatcattcctgaacacactgtagggtgaattggtgcatcttccccaccatacacacacacacacacacacacacacacacacacacacacacaccccaaaccttttcatggggaatgtgtggcaaccttgccaaacagcaccactcagagtgtgactctgactgtgaccttggccttaatgaggaacttcttaggagagtttgaggacaaggccaacatcgtcatctgggctcgctgcgtcccagcacatcaaactctgtccagagacaaggccaactgcaaatgaaagccagggaacattgctaagggtctgtggctctgtggtggtgttcatcgccttcctgagataggatttcccttgccagtcccaacctgtatatattctgtacagaagacatccctgaatatactgtaggtgagtcgtccagccaaatttatatctccaaaacatttttagctttttctacatgctatgaattgagatgacatgctcaacttgtaaataagtctttttgtacattaaaaaagtaattttttcataatttatcttgtctatctgcttcccccttgacagtagttaatgagaacctgggcagtaaatttggtgcattcgagcagaaattaggctgtattttttcttaacagtgtcaaaattgactatcccgcctttgccaagaaatgtttaatgctgaggca

SEQ ID NO: 303 - protein, 767 aa, Homo sapiens
MATEGGKTSEPENNNKKPKTSGSQDSQPSPLALLAATCSKIGTPGENQATGQQQIIIDPSQGLVQLQNQPQQLELVTTQLAGNAWQLVASTPPASKENNVSQPASSSSSSSSSNNGSASPTKTKSGNSSTPGQFQVIQVQNPSGSVQYQVIPQLQTVEGQQIQINPTSSSSLQDLQGQIQLISAGNNQAILTAANRTASGNILAQNLANQTVPVQIRPGVSIPLQLQTLPGTQAQVVTTLPINIGGVTLALPVINNVAAGGGTGQVGQPAATADSGTSNGNQLVSTPTNTTTSASTMPESPSSSTTCTTTASTSLTSSDTLVSSADTGQYASTSASSSERTIEESQTPAATESEAQSSSQLQPNGMQNAQDQSNSLQQVQIVGQPILQQIQIQQPQQQIIQAIPPQSFQLQSGQTIQTIQQQPLQNVQLQAVNPTQVLIRAPTLTPSGQISWQTVQVQNIQSLSNLQVQNAGLSQQLTITPVSSSGGTTLAQIAPVAVAGAPITLNTAQLASVPNLQTVSVANLGAAGVQVQGVPVTITSVAGQQQGQDGVKVQQATIAPVTVAVGGIANATIGAVSPDQLTQVHLQQGQQTSDQEVQPGKRLRRVACSCPNCREGEGRGSNEPGKKKQHICHIEGCGKVYGKTSHLRAHLRWHTGERPFICNWMFCGKRFTRSDELQRHRRTHTGEKRFECPECSKRFMRSDHLSKHVKTHQNKKGGGTALAIVTSGELDSSVTEVLGSPRIVTVAAISQDSNPATPNVSTNMEEF

SEQ ID NO: 304 - DNA, 6072 bp, Homo sapiens
  source 1..6072: mol_type = genomic DNA
acagcccagcggcggccattcgcggaaaaagaggcagagcctgtgccagctacagcctcctccgagccaccgcgggcgggcgggaccggcctctcctcccgcctcgcccccacccccacccacctctatcccagtgtctccgtctgagggtttgtcctgttaatgcgggatgagcgaagaaggaggaggaggaggaggcggcagcggcagcggcgatggctacagaaggagggaaaacctctgagccagagaataacaataaaaaacccaaaacctcaggctcccaggactctcagccctctcctctggctttactggcagctacttgcagcaaaatagggactcctggtgaaaatcaagcaactggacaacaacaaattattatagatccaagtcaaggattggtgcaacttcaaaatcaaccacaacagctagaactggtaacaacgcaacttgctggaaacgcttggcaacttgttgcctccactcctcctgcttcaaaagagaataacgtttctcaaccagcctctagttcgtctagttcttccagcagtaataacgggagtgcatctcctacaaaaactaaatcaggtaattcttccacccctggtcaatttcaagtcatacaagtacaaaatccaagtggtagtgtacagtaccaagtaattccacaacttcagacagtggaaggtcaacaaattcaaatcaatccaactagtagttcatctctacaggatttgcagggtcaaattcagctcatttctgcaggtaataatcaagctatactcacagctgctaacaggacagcttctgggaatattcttgctcaaaacctggcaaatcagacagttccggtccaaattagacctggtgtttcaataccactgcagttacagactcttcctggtactcaggctcaagttgtaacaaccctaccaattaacattggaggagtgactctagctttgccagtgataaacaacgtggctgccggaggagggactgggcaggttggccagcctgctgctactgctgatagtgggacttccaatgggaatcaattagtttccacacccaccaacaccactacttctgccagtactatgccagaatctccctcctcctccactacctgcacaaccactgcttcaacgtctttgacaagcagtgacacattagtgagctcagcagatactggccagtatgcaagcacatcagccagtagttctgaacgcaccattgaagaatctcaaacacctgctgctactgagtctgaagcccagagctccagtcagcttcagcctaatggaatgcagaatgcacaggatcaatcaaattctcttcagcaggtgcaaattgtaggccaacctatcttacagcagatccagatccaacagcctcagcaacagatcattcaggctattccaccacagtcgtttcaactccagtcagggcagacgattcagaccatccagcagcagcctttacagaatgttcaacttcaagcagtaaatccgactcaggtgcttatcagggctccaactttaacaccttcagggcaaatcagttggcaaactgtacaggttcagaatattcagagtctttcaaatttgcaagttcagaatgctgggttatcccaacaattaaccatcaccccagtgtcttcaagtggtggcacaactcttgctcagattgctcctgtggctgttgctggtgccccaataactttgaatactgcccagcttgcatcagtgcctaaccttcagacagtgagcgttgccaacctgggtgctgcaggtgttcaagtgcagggagttcccgttacaatcactagtgttgcaggtcagcagcaaggacaagatggagtaaaagtccagcaagctactatagctcctgtaactgtagcagttggaggaattgctaatgccacgataggtgctgttagtcctgaccaactcacacaagtgcatttgcagcaaggccagcagacttctgatcaagaggtacaacctggcaagaggcttcgaagagttgcctgttcctgtcctaattgtagggaaggagaaggaagaggcagtaatgaaccaggaaaaaagaagcagcatatctgtcatattgaaggatgtggtaaagtttatggcaaaacatctcatttacgagcacatcttcgctggcatactggagaaagaccttttatatgcaactggatgttttgtggcaaaagattcacacggagtgatgagctccagagacatagaagaacccatacaggtgaaaagagatttgaatgcccggaatgttctaaaaggtttatgcggagtgatcatctctccaaacatgtcaaaacgcaccagaataaaaaaggtggtgggacagctcttgccattgttacctcgggagaactggactcatctgttacagaggtgcttggctccccaagaattgtcacagttgcagccatttctcaagattcgaatccagcaactcccaatgtttcaaccaacatggaagaattctgaaaagttatttataacagagacctctagtgctgcacttgtttacacacctttgaaaatctggaaatgggctggtcaagtggattacagagtaggaaattatgttttcattcttggcttctttaagtattccagggtttggggtcaacacgtgaagtgttgaattttaaaaaatacaaaaagcagactgatgtactggaaacagaaaagtatttcctccatactataagttgtagttgtttggaaatatatcacataacctttatacagaatcttcccatctcttaatatcatgtgttaacatgtttaaaaagaccttagtagtttgcaggctggaccttaattggacttattttctttgaaagtactttgttataaattcagtcagtaataatttacgtgtattctttttctctatagcacagaaaacagatagttaactgatgatagggataatactgtatttccttagcttgatttttggaaaatcaaccgaaaatagtttggccgtcttttctaaatgttagaaattcttcaacagttgaattaggtaagttccaaaacagtaatctgagatgcatctcagatctttattaccactacattatagtagtgtgtatgcagacaatcagtgaagtccaattactttctccatttggagacacaagaggaacatagagttaaatcttaggttaaattttaggttgacaccttaggaaaatgctgggaaaaaaatggttaaaacaaaactcatcatagcttcagaaaaataaaatgaggcatcttaacatgcaatgttctaaagttaggattgattatattcctaaccctaggttgaaccacaaaatttcatttaaaatgtttatatttggaaatatttgcatagagtgtaaattgttctgtagtttcatattttgtaaatatgagttatgttgacaatgtgcagaattctttatgctttgatgtggtagccaaagaaagaattacacttttttccaaggccagcagaaaattctcttttaactacattgtaattcttgttttcctctactaaaaattggccagtcccattttatttctagtgctatgtaagaaggtaattaggaattataacacagtaatgtttttatgttacatcaataactgaattttccctaaaaattagcctaatatataatagatatattatgaagcaaaacttttatttttgaaaaggcagaataattttcagtgaagtaagtgactaaagaaaaaaactatattattgtttatgcaagggtcttacaggaaagggtctttttttttttttttttgagatggagtctcgctctcttgcccaggctggagtgcaatggcacgatctcagcttactgcaaccgccgcctcccaggttcaagcgattctcctgtcttagcctcctgagtagctgggattaacaggcgcctgccaccatgcctggctaatttttgtatttttagtagagacagggtttcgccatgttggccaggctggtttcaaactcctgacctcaggtgatctgcccacctcggcctcccagagtgctgggattacaggcatgagccaccatgcccggctaggaaagggtcttactaggaaagatggccaaaagtttcatatgaaaaaaattggattataaaccagtaacttaaatattaataaggatattttatgttttaaaaaagtatttacacagaatcataatcagtgaaattgaccatttgaaaactaaaagtttttacctacctgctcaatttattaacatcattgctttggggactgtttgaatataggtacgtgttttcttgtgcattctctataatttcaggaaaagtactaatgcgaattctcttccaaaattgtgatgtttcttgtatttttgatgaaggagaaatactgtaatgatcactgtttacactatgtacactttaggccagccctttgtagcgttatataaaactgaaggtcttttgtgctttcagtttgtataaaaaagctttgagattaaaggaaaaaaaaaatttttacactgtggttataaatttcaagtttcttaaagcttttgtagacttgtaacagagtctttaaatttaagttggattttgtaaattgttttgtatattttatttaatgtactcttaacaactgatgtatctggctttaaaacatcagaattggtttgttgttttgtttggtacagaggagcatttggtagtgttcattttaattttattatataggagctgaaacatcaaatatatattttatctatcattatgctacacaatcagtgctataaatttttttatgcaaagaaactttcttaatgttttaatcatgtttcctcagagtgacactttttgttgttgttaataccaagtatgaacactctgcatcattattccatgaaccagttctaatgcaaacctatgtatgtccattagaaatggaagttattttttaatcaacaatgaggcctattataaatttatcagatgaatctagatagctttatagcatataaaatatgttaatttgtgttagcaggtgcacatttcaccactgaaattagaaatattttgacagtctgttctgcataccattctgagtctacttttctgtctttagaagaatcgtaaatttcagtgtcctttatttgactcagtgggatatagctgttataagtaatagggcacagatgtgcagtagagtcttgtttaatggcatttcactgttcattccctttaccaccgttataaaacttttctttattgtaattatcagtgcaaagctatgtatttatcatggtaaaactccagtgttagaatagttttttcttacagtatactttctttggttaggtttgtgtatgtgttgctgattacattagaacttgatgttaagtcatttatcacactctcatgagagcagtaataaaagtgtgtaatctaggagaaaaagttaatttgtcaaacttagataagcatgatgtttaggtcctatttttcaattttataactgttttattgcaacaatatttgtatttaagtctccattttaatgccttgtggtgtttttttatgcatgtcactaagttgtcatcccacataaattgatgtgcagcatagggtattaaatctacataatgattttaaaacagaaatagttgatggtaaaatgtaaatgttttgcaaaaattccttataaaaagttttgtagtaacatttcacttgtaaattttttttgtaaaaaaaaaaaaatgaaaaaaaaagatgaatccagaaaaaaacctgtttcccatattctagaatttagacaattattctgccagcaaagcctctggggctgtaattgacatttttacagtgctgatttgtataaaatttgttttttgtggatttggaaataaaatcatgtacaagttgttgcctgcaataacaattgcaagtaacctattaaaaattcccttgagtttaacatgtttcatttaattatgtatactataaagcagcaataaattatttgaactatcaaccta

SEQ ID NO: 305 - protein, 505 aa, Homo sapiens
MKFKLHVNSARQYKDLWNMSDDKPFLCTAPGCGQRFTNEDHLAVHKHKHEMTLKFGPARNDSVIVADQTPTPTRFLKNCEEVGLFNELASPFENEFKKASEDDIKKMPLDLSPLATPIIRSKIEEPSVVETTHQDSPLPHPESTTSDEKEVPLAQTAQPTSAIVRPASLQVPNVLLTSSDSSVIIQQAVPSPTSSTVITQAPSSNRPIVPVPGPFPLLLHLPNGQTMPVAIPASITSSNVHVPAAVPLVRPVTMVPSVPGIPGPSSPQPVQSEAKMRLKAALTQQHPPVTNGDTVKGHGSGLVRTQSEESRPQSLQQPATSTTETPASPAHTTPQTQSTSGRRRRAANEDPDEKRRKFLERNRAAASRCRQKRKVWVQSLEKKAEDLSSLNGQLQSEVTLLRNEVAQLKQLLLAHKDCPVTAMQKKSGYHTADKDDSSEDISVPSSPHTEAIQHSSVSTSNGVSSTSKAEAVATSVLTQMADQSTEPALSQIVMAPSSQSQPSGS

SEQ ID NO: 306 - DNA, 4199 bp, Homo sapiens
  source 1..4199: mol_type = genomic DNA
gtcagtccgatctcgcgagagaggacggaagcctgtgggagcccgtggcctttaaagtgccgttcagccttttcctccaggggtgctttgtaaacacggctgtgctcagggctcgcgggtgaccgaaaggatcatgaactagtgacctggaaagggtactagatggaaacttgagaaaggactgcttattgataacagctaaggtattcctggaagcagagtaaataaagctcatggcccaccagctagaaagatttcctgaagaggtagtctgattggcttaactcgtattcttgccatgagaaaaagaatgtgataagttattcaacttatgaaattcaagttacatgtgaattctgccaggcaatacaaggacctgtggaatatgagtgatgacaaaccctttctatgtactgcgcctggatgtggccagcgttttaccaacgaggatcatttggctgtccataaacataaacatgagatgacactgaaatttggtccagcacgtaatgacagtgtcattgtggctgatcagaccccaacaccaacaagattcttgaaaaactgtgaagaagtgggtttgtttaatgagttggcgagtccatttgagaatgaattcaagaaagcttcagaagatgacattaaaaaaatgcctctagatttatcccctcttgcaacacctatcataagaagcaaaattgaggagccttctgttgtagaaacaactcaccaggatagtcctttacctcacccagagtctactaccagtgatgagaaggaagtaccattggcacaaactgcacagcccacatcagctattgttcgtccagcatcattacaggttcccaatgtgctgcttacaagttctgactcaagtgtaattattcagcaggcagtaccttcaccaacctcaagtactgtaatcacccaggcaccatcctctaacaggccaattgtccctgtaccaggcccatttcctcttctgttacatcttcctaatggacaaaccatgcctgttgctattcctgcatcaattacaagttctaatgtgcatgttccagctgcagtcccactcgttcgaccagtcaccatggtgcctagtgttccaggaatcccaggtccttcctctccccaaccagtacagtcagaagcaaaaatgagattaaaagctgctttgacccagcaacatcctccagttaccaatggtgatactgtcaaaggtcatggtagcggattggttaggactcagtcagaggaatctcgaccgcagtcattacaacagccagccacatccactacagaaactccggcttctccagctcacacaactccacagacccaaagtacaagtggtcgtcggagaagagcagctaacgaagatcctgatgaaaaaaggagaaagtttttagagcgaaatagagcagcagcttcaagatgccgacaaaaaaggaaagtctgggttcagtctttagagaagaaagctgaagacttgagttcattaaatggtcagctgcagagtgaagtcaccctgctgagaaatgaagtggcacagctgaaacagcttcttctggctcataaagattgccctgtaaccgccatgcagaagaaatctggctatcatactgctgataaagatgatagttcagaagacatttcagtgccgagtagtccacatacagaagctatacagcatagttcggtcagcacatccaatggagtcagttcaacctccaaggcagaagctgtagccacttcagtcctcacccagatggcggaccagagtacagagcctgctctttcacagatcgttatggctccttcctcccagtcacagccctcaggaagttgattaaaaacctgcagtacaacagttttagatactcattagtgacttcaaagggaaatcaaggaaagaccagtttccatttatgcgaaatctgtggttgtaaattttttttttttacttgaaattaaatttggctctaaagttggtgtagcagcagttgatcagactgaaaaacggtttttagtctctggaaaaagactgattttgctttttttataaatattattagatttattaatttttctgtgctcaatgtgtaaattgtattataattcattgtgatttatttcacttttaatttgctggtgttttaataaatgggggtgttactgaatctttcttcccacttccatttcttttgaccaccccttaaccctcaactgtgacggtagtagtattatcatttataccaaagttttgcatagtccctgttgactttgtaatgttaacggagtcataaaagcactaggcaagagaaagatagaaatttgcttttaatctttttgccttttattttgcacattatgcaaaaggaaaaacattaaagaacacttttttttaagtgagtgaaaacatggtaaagacatacagtgcttttatgcacattgttaagctaaatcaaggtcatttataatcattttccttttttatttaagattttaagtaaacaaattttagaattttcagcatttcaaaaatgattttatttttcaagtcttaaattcaatattttacacctatgttttgaggctaaaaatatgaaattatataatgtatgatacagggttatcaaatatctaataatttttgaaattagctcttgtttttggtttttttgttgttgtttttacagatttcaggttacaaactgcaaagtttatgcataattaagtatggtatggttgccagaaaagcctaaaattactacttagaaaatttaagactgtttacccccattgtcttgtacttgcgagctaacttgtactattcttgtgaaagcactgtcatcttttagtagcaaattttgataatgtttctcgtggaaaaaaaaatcagtatctatctttagaacaatgtaattataatgtgggaagtgtgcatgaatgagagagagtgtgtgtgtatctgtgtgtgtgtgcgcgtgtgtgtgtgtcttttaatagtttatgccagcaatctttgcttgaatgtttaacgatgccttcagtgtgatgctggccaatagatgattgcagtttaaaatgtcattattgtgcaggcttggataactaacattccatgatgtagcttgtttctgatgagatgattgtaggtacatttttctcattatccaatcatctgtgggatacttagttttctaatgtgccattatctatttttattctgcagttatgttcaaaatacagtacatattttaaaatagaataaattgttaaacataaaattttaaaagtagtagatgtgcgtaagaaaactttgtaaaatagttatgagtcctacccagtagcaacttctggcattcaagcaggattccactatgtaaatatctgtaatgcatttataataagttgtgtagtttgtcctgcatccatactacactatttgctaaagtctcagtgccatctcctaatgagactgacattttaaaagtctgtatggaatatccttgataattcaaggaaatatccctcctgcctaagttccaaactgggaaacattcaaattatataaatgacatttcaggactttaagtatgaagataatgggaattttattgttttgctttttaaaatgagagcatttttatttgataattttttttaaatttttaatttttaactaatttcattattttaaagtaatcagtttttcaaatcatgattttgatatcattattctaaggagttatctcaaaggcacaaaatatgaattctgcaagaaggctattttttattgtagtttgaatgggttaggaaaagcctcaatttttcactcttaagtccttcagtacatttttctttcatcttattacttatgcaagttaaggttctttggtaacagaattcttgcaactgtaaaataaaactacatagatgtaagaagtcatgtaaacggttaacaagcttaccaaggttagcaaaactttcattgtaaatcagtctgtactgagcaaataaaaatcattattagttgtataaacacaaattccattttgactttcaggatgtcatactacttctgtacctagcattttcagtccttatatttgcaatgttacacaaactgtactattttcttttatgtgcagtttgcatgagtaaaccatcagagaataaattctatctttaaatta

SEQ ID NO: 307 - protein, 181 aa, Homo sapiens
MMLQHPGQVSASEVSASAIVPCLSPPGSLVFEDFANLTPFVKEELRFAIQNKHLCHRMSSALESVTVSDRPLGVSITKAEVAPEEDERKKRRRERNKIAAAKCRNKKKEKTECLQKESEKLESVNAELKAQIEELKNEKQHLIYMLNLHRPTCIVRAQNGRTPEDERNLFIQQIKEGTLQS

SEQ ID NO: 308 - DNA, 1935 bp, Homo sapiens
  source 1..1935: mol_type = genomic DNA
ggcggaggtggggttagcttcagttgaccaaccatgccttgaggataaattggatgggatcagatgggaagatgtgacaagaagagaaatcctcctctatataggatgctctgctgtttcctaaggattttcagcaccttgccccaaaatcaaaatgatgcttcaacacccaggccaggtctctgcctcggaagtgagtgcttctgccatcgtcccctgcctgtcccctcctgggtcactggtgtttgaggattttgctaacctgacgccctttgtcaaggaagagctgaggtttgccatccagaacaagcacctctgccaccggatgtcctctgcgctggaatcagtcactgtcagcgacagacccctcggggtgtccatcacaaaagccgaggtagcccctgaagaagatgaaaggaaaaagaggcgacgagaaagaaataagattgcagctgcaaagtgccgaaacaagaagaaggagaagacggagtgcctgcagaaagagtcggagaagctggaaagtgtgaatgctgaactgaaggctcagattgaggagctcaagaacgagaagcagcatttgatatacatgctcaaccttcatcggcccacgtgtattgtccgggctcagaatgggaggactccagaagatgagagaaacctctttatccaacagataaaagaaggaacattgcagagctaagcagtcgtggtatgggggcgactggggagtcctcattgaatcctcattttatacccaaaaccctgaagccattggagagctgtcttcctgtgtacctctagaatcccagcagcagagaaccatcaaggcgggagggcctgcagtgattcagcaggcccttcccattctgccccagagtgggtcttggaccagggcaagtgcatctttgcctcaactccaggatttaggccttaacacactggccattcttatgttccagatggcccccagctggtgtcctgcccgcctttcatctggattctacaaaaaaccaggatgcccaccgttaggattcaggcagcagtgtctgtacctcgggtgggagggatggggccatctccttcaccgtggctaccattgtcactcgtaggggatgtggagtgagaacagcatttagtgaagttgtgcaacggccagggttgtgctttctagcaaatatgctgttatgtccagaaattgtgtgtgcaagaaaactaggcaatgtactcttccgatgtttgtgtcacacaacactgatgtgacttttatatgctttttctcagatctggtttctaagagttttggggggcggggctgtcaccacgtgcagtatctcaagatattcaggtggccagaagagcttgtcagcaagaggaggacagaattctcccagcgttaacacaaaatccatgggcagtatgatggcaggtcctctgttgcaaactcagttccaaagtcacaggaagaaagcagaaagttcaacttccaaagggttaggactctccactcaatgtcttaggtcaggagttgtgtctaggctggaagagccaaagaatattccattttcctttccttgtggttgaaaaccacagtcagtggagagatgtttggaaaccacagtcagtggagcctgggtggtacccaggctttagcattattggatgtcaatagcattgtttttgtcatgtagctgttttaagaaatctggcccagggtgtttgcagctgtgagaagtcactcacactggccacaaggacgctggctactgtctattaaaattctgatgtttctgtgaaattctcagagtgtttaattgtactcaatggtatcattacaattttctgtaagagaaaatattacttatttatcctagtattcctaacctgtcagaataataaatattggaaccaagacatggtaaacaaaaaaaaaaaaaa

SEQ ID NO: 309 - protein, 510 aa, Homo sapiens
MDGFYDQQVPFMVPGKSRSEECRGRPVIDRKRKFLDTDLAHDSEELFQDLSQLQEAWLAEAQVPDDEQFVPDFQSDNLVLHAPPPTKIKRELHSPSSELSSCSHEQALGANYGEKCLYNYCAYDRKPPSGFKPLTPPTTPLSPTHQNPLFPPPQATLPTSGHAPAAGPVQGVGPAPAPHSLPEPGPQQQTFAVPRPPHQPLQMPKMMPENQYPSEQRFQRQLSEPCHPFPPQPGVPGDNRPSYHRQMSEPIVPAAPPPPQGFKQEYHDPLYEHGVPGMPGPPAHGFQSPMGIKQEPRDYCVDSEVPNCQSSYMRGGYFSSSHEGFSYEKDPRLYFDDTCVVPERLEGKVKQEPTMYREGPPYQRRGSLQLWQFLVTLLDDPANAHFIAWTGRGMEFKLIEPEEVARRWGIQKNRPAMNYDKLSRSLRYYYEKGIMQKVAGERYVYKFVCDPDALFSMAFPDNQRPFLKAESECHLSEEDTLPLTHFEDSPAYLLDMDRCSSLPYAEGFAY

SEQ ID NO: 310 - DNA, 4082 bp, Homo sapiens
  source 1..4082: mol_type = genomic DNA
agagtccagccgctggtgcgcggagcggttcaccgtcttcggagcggttcggcccagcctttcgcccaggcgcccaggcccgctgcgcgcgtgcgtgagcgcgcctgcgccgccggggccgctgcaaggggaggagagaggccgcctcaggaggatcccttttcccccagaaattactcaatgctgaaacctctcaaagtggtattagagacgctgaaagcaccatggacgggttttatgatcagcaagtcccttttatggtcccagggaaatctcgatctgaggaatgcagagggcggcctgtgattgacagaaagaggaagtttttggacacagatctggctcacgattctgaagagctatttcaggatctcagtcaacttcaagaggcttggttagctgaagcacaagttcctgatgatgaacagtttgtcccagattttcagtctgataacctggtgcttcatgccccacctccaaccaagatcaaacgggagctgcacagcccctcctctgagctgtcgtcttgtagccatgagcaggctcttggtgctaactatggagaaaagtgcctctacaactattgtgcctatgataggaagcctccctctgggttcaagccattaacccctcctacaacccccctctcacccacccatcagaatcccctatttcccccacctcaggcaactctgcccacctcagggcatgcccctgcagctggcccagttcaaggtgtgggccccgcccccgccccccattcgcttccagagcctggaccacagcagcaaacatttgcggtcccccgaccaccacatcagcccctgcagatgccaaagatgatgcctgaaaaccagtatccatcagaacagagatttcagagacaactgtctgaaccctgccaccccttccctcctcagccaggagttcctggagataatcgccccagttaccatcggcaaatgtcagaacctattgtccctgcagctcccccgccccctcagggattcaaacaagaataccatgacccactctatgaacatggggtcccgggcatgccagggcccccagcacacgggttccagtcaccaatgggaatcaagcaggagcctcgggattactgcgtcgattcagaagtgcctaactgccagtcatcctacatgagagggggttatttctccagcagccatgaaggtttttcatatgaaaaagatccccgattatactttgacgacacttgtgttgtgcctgagagactggaaggcaaagtcaaacaggagcctaccatgtatcgagaggggcccccttaccagaggcgaggttcccttcagctgtggcagttcctggtcacccttcttgatgacccagccaatgcccacttcattgcctggacaggtcgaggcatggagttcaagctgatagaaccggaagaggttgctcggcgctggggcatccagaagaaccggccagccatgaactatgacaagctgagccgctctctccgctattactatgaaaagggcatcatgcagaaggtggctggagagcgatacgtctacaaatttgtctgtgacccagatgccctcttctccatggctttcccggataaccagcgtccgttcctgaaggcagagtccgagtgccacctcagcgaggaggacaccctgccgctgacccactttgaagacagccccgcttacctcctggacatggaccgctgcagcagcctcccctatgccgaaggctttgcttactaagtttctgagtggcggagtggccaaaccctagagctagcagttcccattcaggcaaacaagggcagtggttttgtttgtgtttttggttgttcctaaagcttgccctttgagtattatctggagaacccaagctgtctctggattggcacccttaaagacagatacattggctggggagtgggaacagggaggggcagaaaaccaccaaaaggccagtgcctcaactcttgattctgatgaggtttctgggaagagatcaaaatggagtctccttaccatggacaatacatgcaaagcaatatcttgttcaggttagtacccgcaaaacgggacatagtatgtgacaatctgcatcgatcatggactactaaatgcctttacatagaagggctctgatttgcacaatttgttgaaaaatcacaaacccatagaaaagtaagtaggctaagttggggaggctcaaaccattaagggttaaaaatacatcttaaacattggaaagctcttctagctgaatctgaaatattaccccttgtctagaaaaaggggggcagtcagaacagctgttccccactccgtggttctcaaaatcataaaccatggctactcttgggaaccacccggccatgtggtcgccaagtagagcaagccccctttctcttcccaatcacgtggctgagtgtggatgacttttattttaggagaagggcgattaacacttttgacagtattttgttttgccctgatttgggggattgttttgttttggtggttgttttggaaaaacagtttataaactgatttttgtagttttggtatttaaagcaaaaaaacgaaaaacaaaaaacaaaaacaaaccttttggtaactgtgcactgtgtcctttagccagggccgtgccaacttatgaagacactgcagcttgagaggggctttgctgaggcttccccttggccatgtgaaagcccgccttgttgcctgctttgtgctttctgcaccagacaacctgatggaacatttgcacctgagttgtacatttttgaagtgtgcagggcagcctggacacaagcttagattctctatgtatagttccccgtgttcactaacatgccctctctggaaagcatatgtatataacatgtgtcatgtcctttggaaacctggtcacctggtgaaaacccttgggattcttccctgggcatgactgatgacaatttccatttcatcagtttgttttgttttcctttttctttaaatcttggactttaaaccctacctgtgtgattcagtagggtttgagacttacgtgtgatactgacaggtaagcaacagtgctagcattctagattcctgcctttttttaaaaagaaattattctcattgctgtattatattggaaaagttttaaacaaccaagctaaagctatgtgaaagttgagctcaaagtagaggaaaagttactggtggtaccttgctgcctgctctgctggtagaattctgtgctccccgtgacacttagtacattaagaatgactacactgttcctcgtatgtgaaggaggcagtgctgactccgtgagtgtgagacacgtgctttgaactgcttttctattcatggagcactccatagtctcaaactgtcccccttatgaccaacagcacatttgtgaagaggttcgcagggataaggggtgcactttatagctatggaaacatgagattctcctctattggaagctaattagcccacaaaggtggtaaacctgtagattgggccttaattagcattgtactctaatcaaaggactctttctaaaccatatttatagctttcttaacctacacatagtctatacatagatgcatattttacccccagctggctagagatttatttgttgtaaatgctgtatagatttggttttcctttctttacttaccctggtttggattttttttttttttcttttgaatggatttatgctgtcttagcaatatgacaataatcctctgtagcttgagctacccctcccctgctgtaacttacgtgacctgtgctgtcactgggcataggacagcggcatcacggttgcattcccattggactcatgcacctcccggatggtttttgtttttttcgggggttctttggggtttgtttgtttgcttcttttccagagtgtggaaagtctacagtgcagaaaggcttgaacctgccagctgatttgaaatactttcccctgcgcagggccgtatgcatcctgccaagctgcgttatattctgtactgtgtacaataaagaagtttgcttttcgtttaccaagca

(19) United States
(12) Patent Application Publication
(10) Pub. No.: US 2026/0097131 A1
(43) Pub. Date: Apr. 9, 2026

(54) COMPOSITIONS AND METHODS FOR REPROGRAMMING DISEASED MUSCULOSKELETAL CELLS

(71) Applicants: Ohio State Innovation Foundation, Columbus, OH (US); UMIP The University of Manchester Intellectual Property, Manchester (GB)

(72) Inventors: Devina WALTER, Columbus, OH (US); Shirley TANG, Groveport, OH (US); Judith HOYLAND, Hadfield (GB); Safdar KHAN, Columbus, OH (US); Benjamin WALTER, Columbus, OH (US); Daniel GALLEGO-PEREZ, Columbus, OH (US); Natalia HIGUITA-CASTRO, Columbus, OH (US)

(21) Appl. No.: 19/415,812

(22) Filed: Dec. 11, 2025

Related U.S. Application Data
(62) Division of application No. 17/332,470, now Pat. No. 12,508,325.
(60) Provisional application No. 62/782,734, filed on Dec. 20, 2018.

(51) Int. Cl. A61K 48/00; A61P 21/00; C07K 14/47; C12N 15/85

(52) U.S. Cl. CPC A61K 48/0025; A61P 21/00; C07K 14/4702; C12N 15/85; C12N 2800/107

(57) ABSTRACT

Disclosed herein are compositions and methods for reprogramming diseased musculoskeletal cells both in vitro and in vivo. In some embodiments, the disclosed method involves non-virally delivering intracellularly into the diseased musculoskeletal cells a polynucleotide comprising one or more nucleic acid sequences encoding one or more of the disclosed transcription factors.

### CROSS-REFERENCE TO RELATED APPLICATIONS

This application is a divisional of copending application Ser. No. 17/332,470, filed May 27, 2021, which is a National Stage of International Application No. PCT/US2019/067448, filed Dec. 19, 2019, which claims benefit of U.S. Provisional Application No. 62/782,734, filed Dec. 20, 2018, which are hereby incorporated herein by reference in their entireties.

### SEQUENCE LISTING

This contains a sequence listing filed in ST.26 format entitled “OSU_T2019_151_DIV_US_Sequence_Listing” created on Dec. 1, 2025 and having 689,190 bytes. The content of the sequence listing is incorporated herein in its entirety.

### BACKGROUND

Current therapies for musculoskeletal diseases, such as low back pain, are highly invasive and is a major contributor to the growing opioid crisis. Additionally, these therapies only treat the symptomatic pain of the patient while failing to target the underlying pathology of disease which leads to further disease progression and future pain. For example, lumbar fusion of the spine is a common surgical operation to fuse the spine in place of the intervertebral disc space between the vertebras. However, removal of the disc and fusion of the vertebrae often results in adjacent segment disease due to imbalanced biomechanics of the spine post-surgery. In addition, micro-discectomies which remove the diseased tissue from the site often lead to tissue collapse and additional surgical intervention with added pain. Therefore, new treatment methods of such diseases are needed to alleviate these issues.

Current studies in the field include engineered intervertebral discs, cell therapies, drug delivery, growth factors, viral reprogramming or gene editing. However, these all include their pitfalls and risks. Engineered constructs for replacement of musculoskeletal components are disadvantageous in their biocompatibility and most importantly mechanical integrity in the body environment to function effectively. Cell therapies are poor in terms of long-term cell viability due to the harsh avascular environment of tissues such as the intervertebral disc. Drug delivery systems are hard to sustain in the environment and has potential to leech onto nearby tissue with undesired effects similar to growth factors such as Bone morphogenic proteins (BMPs) and Tumor Growth Factor (TGFβ). Viral reprogramming and gene editing have large regulatory burdens as they often involve integration into the native host genome which has been shown in history to cause adverse immunogenic and mutagenic effects on the patients. The death of Jesse Gelsinger is one such example.

### SUMMARY

Disclosed herein are compositions and methods for reprogramming diseased musculoskeletal cells both in vitro and in vivo.

In some embodiments, the disclosed method involves non-virally delivering intracellularly into the diseased musculoskeletal cells a polynucleotide comprising one or more nucleic acid sequences encoding one or more transcription factors, such as HIF-1a, FOX, T, SOX, and Mohawk families of transcription factors, including the factors listed in Tables 1A, 1B, and 1C.

For example, in some embodiments, the method involves reprogramming a diseased nucleus pulposus (NP) cell into a healthy cell by non-virally delivering intracellularly into the NP cell one or more transcription factor proteins selected from the group comprising HIF-1a, HIF-2a, Hedgehog family (SHH, DHH, IHH), a T-box family of proteins (TBXT, TBR1, TBX1-6, TBX10, TBX15, TXB18-22), and a Forkhead-box (FOX) family of proteins (FOXF1, FOXA1-3, FOXB1-2, FOXC1-2, FOXD1-6, FOXE1-3, FOXG1, FOXH1, FOXI1, FOXJ1, FOXK1, FOXL1-2, FOXM1, FOXN1-4, FOXO1, FOXO3-4, FOXO6, FOXP1-4, FOXQ1, FOXR1-2), or polynucleotides encoding the one or more transcription factor proteins; or exposing the NP cell to an extracellular vesicle produced from a cell containing or expressing the one or more transcription factor proteins, or polynucleotides encoding the one or more transcription factor proteins.

In some embodiments, the method involves reprogramming a diseased annulus fibrosis (AF) cell into a healthy cell by non-virally delivering intracellularly into the AF cell one or more transcription factor proteins selected from the group comprising a Iroquois Homeobox family of proteins (Mohawk, IRX1-6), Tenomodulin and Scleraxis, or polynucleotides encoding the one or more transcription factor proteins; or exposing the AF cell to an extracellular vesicle produced from a cell containing or expressing the one or more transcription factor proteins, or polynucleotides encoding the one or more transcription factor proteins.

In some embodiments, the method involves reprogramming a diseased cartilage endplate cell into a healthy cell by non-virally delivering intracellularly into the cartilage endplate cell one or more transcription factor proteins selected from the group comprising an NFAT Family proteins (NFATc1-4), ERG (C-1-1), PGC1a, Osterix, SOX family of proteins (SRY, SOX1-15, SOX17-18, SOX21, SOX30) and MEF2C, or polynucleotides encoding the one or more transcription factor proteins; or exposing the cartilage endplate cell to an extracellular vesicle produced from a cell containing or expressing the one or more transcription factor proteins, or polynucleotides encoding the one or more transcription factor protein.

Also disclosed herein is a method for treating a musculoskeletal disease in a subject that involves non-virally delivering intracellularly into disease musculoskeletal cells of the subject one or more transcription factor proteins selected from the group comprising HIF-1α, HIF-2α, a T-box family protein, and Forkhead-box (FOX) family protein, a Iroquois family proteins, Tenomodulin, Scleraxis, NFAT Family proteins, ERG, PGC1α, Osterix, Runx family of proteins, Hedgehog family of proteins, SOX family of proteins and MEF2C, or polynucleotides encoding the one or more transcription factor proteins; or exposing the disease musculoskeletal cells to an extracellular vesicle produced from a cell containing or expressing the one or more transcription factor proteins, or polynucleotides encoding the one or more transcription factor proteins.

In some embodiments, the musculoskeletal disease is osteoarthritis where chondrocytes, synoviocytes, fibrocartilage cells of the meniscus, osteoblasts, osteocytes and osteoclasts will be subject to non-viral reprogramming. In some embodiments, the musculoskeletal disease is intervertebral disc degeneration and chronic low back pain where notochordal cells, nucleus pulposus cells, annulus fibrosus cells, cartilage endplate cells, ligamentous cells, dorsal root ganglion cells and myocytes/myofibroblasts will be subject to non-viral reprogramming or injection of engineered vesicles. In some embodiments, the musculoskeletal disease is tendinopathy or rotator cuff tendonitis where tenocytes and myocytes/myofibroblasts will be subject to non-viral reprogramming or injection of engineered vesicles.

In some embodiments, the disclosed methods involve non-viral tissue nanotransfection (TNT) of notochordal cells, nucleus pulposus (NP), annulus fibrosis (AF), or cartilage endplate cells of a subject's intervertebral disc (IVD) or chondrocytes, synoviocytes, fibrocartilage cells of the meniscus, ligamentous cells, dorsal root ganglion cells, osteoblasts, osteoclasts, osteocytes, myocytes/myofibroblasts, haemapoetic and mesenchymal stem cells or tenocytes. This can be done via direct tissue nanotransfection of the NP, AF, and CEP tissue with previously stated transcription factors during patient surgery, or on cells isolated from patients. More precisely, the tissue nanotransfection device chip will be placed at the site of the IVD where degeneration is occurring and transcription factors targeting the specific tissue will be delivered in-situ. More precisely, cells from the patient IVD can be isolated and transfected ex-vivo with transcription factors and injected back into the patient.

In some embodiments, the disclosed methods involve delivery of extracellular vesicles (EVs) to the notochordal cells, nucleus pulposus (NP), annulus fibrosis (AF), or cartilage endplate cells of a subject's intervertebral disc (IVD) or chondrocytes, synoviocytes, fibrocartilage cells of the meniscus, ligamentous cells, dorsal root ganglion cells, osteoblasts, osteoclasts, osteocytes, myocytes/myofibroblasts, or tenocytes. EVs will be generated using the patient's cells which encapsulates the desired transcription factors specific for each tissue. EVs containing these factors are then injected back into the diseased/degenerate tissue and up taken by the patients cells within 4-6 hours of cell-vector contact.

Also disclosed herein are polynucleotides comprising one, two, or more nucleic acid sequences encoding transcription factors disclosed herein, such as Forkhead-box (FOX) family protein, Iroquois I family proteins, Scleraxis, NFAT Family proteins, ERG, PGC1α, Osterix, and MEF2C. In some embodiments, the transcription factors are mammalian proteins, such as human proteins.

Also disclosed a composition comprising a polynucleotide comprising one, two, or more nucleic acid sequences encoding transcription factors disclosed herein. Also disclosed are non-viral vectors containing the disclosed polynucleotides. In particular embodiments, the vector is a recombinant bacterial plasmid. For example, in some embodiments, the non-viral vector has a pCDNA3 backbone. In some embodiments, the vector comprises an internal ribosome entry site (IRES).

In some embodiments, after transfecting target cells with nucleic acid sequences encoding the disclosed transcription factors, the cells can then pack the transfected genes (e.g. cDNA) into EVs, which can then reprogram diseased musculoskeletal cells. Therefore, also disclosed is a method of reprogramming diseased musculoskeletal cells that involves exposing the cells with an extracellular vesicle produced from a cell containing or expressing the disclosed transcription factors.

In these embodiments, the polynucleotides and compositions may be delivered to diseased musculoskeletal cells, or donor cells, intracellularly via a gene gun, a microparticle or nanoparticle suitable for such delivery, transfection by electroporation, three-dimensional nanochannel electroporation, a tissue nanotransfection device, a liposome suitable for such delivery, or a deep-topical tissue nanoelectroinjection device. In some of these embodiments, the polynucleotides can be incorporated into a non-viral vector, such as a bacterial plasmid. In some embodiments, a viral vector can be used. For example, the polynucleotides can be incorporated into a viral vector, such as an adenoviral vector. However, in other embodiments, the polynucleotides are not delivered virally.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B illustrate an embodiment the disclosed technology to use the combination of transcription factor and TNT/Evs to revert diseased intervertebral disc cells to a healthy phenotype. We have shown promising in-vitro cellular work, in-vivo rodent models, and have submitted grants to move to a large scale in-vivo canine model and larger animal trials in the future clinically relevant to the human condition. Remaining claims will focus on our current in-vitro and in-vivo work

FIG. 2 is a schematic of DNA bulk electroporation into NP cells then seeded in Agarose Gel.

FIG. 3 is a graph showing qPCR Gene expression data validating that the transcription factor was successfully transmitted. X-axis=type of tissue and transcription factor. Colors indicate the gene being tested for.

FIG. 4 contains representative viability images (4× Stitched) of Gels at day 0 and 4 Weeks. (Green=Live, Red=Dead).

FIGS. 5A and 5B are graphs showing Brachyury T expression in autopsy (FIG. 5A) and surgical (FIG. 5B) nucleus pulposus cells after sham or FOXF1 treatment. FIGS. 5C and 5D are graphs showing FOXF1 (FIG. 5C) and KRT19 (FIG. 5D) expression in healthy nucleus pulposus cells after sham or FOXF1 treatment.

FIGS. 6A and 6B are graphs showing ACAN (FIG. 6A) and COL2 (FIG. 6B) expression in healthy nucleus pulposus cells after sham or FOXF1 treatment.

FIGS. 7A and 7B are graphs showing NGF expression in autopsy (FIG. 7A) and surgical (FIG. 7B) nucleus pulposus cells after sham or FOXF1 treatment.

FIGS. 8A and 8B are graphs showing IL1-β expression in autopsy (FIG. 8A) and surgical (FIG. 8B) nucleus pulposus cells after sham or FOXF1 treatment. FIG. 8C is a graph showing IL6 expression in nucleus pulposus cells after sham or FOXF1 treatment of surgical tissue.

FIGS. 9A and 9B are graphs showing MMP12 expression in autopsy (FIG. 9A) and surgical (FIG. 9B) nucleus pulposus cells after sham or FOXF1 treatment. FIGS. 9C and 9D are graphs showing MMP13 expression in autopsy (FIG. 9C) and surgical (FIG. 9D) nucleus pulposus cells after sham or FOXF1 treatment.

FIGS. 10A and 10B are bar graphs showing GAG content in autopsy (FIG. 10A) and surgical (FIG. 10B) nucleus pulposus cells after sham or FOXF1 treatment.

FIGS. 11A and 11B are bar graphs showing KRT19 gene expression at day 0, week 2, and week 4 of BrachT transfected groups normalized to SHAM for non-degenerate (ND, FIG. 11A) and painful-degeneration (PD, FIG. 11B) groups. *p<0.05.

FIGS. 12A and 12B are bar graphs showing ACAN gene expression at day 0, week 2, and week 4 of BrachT transfected groups normalized to SHAM for non-degenerate (ND, FIG. 12A) and painful-degeneration (PD, FIG. 12B) groups. *p<0.05.

FIGS. 13A and 13B are bar graphs showing MMP13 gene expression at day 0, week 2, and week 4 of BrachT transfected groups normalized to SHAM for non-degenerate (ND, FIG. 13A) and painful-degeneration (PD, FIG. 13B) groups. *p<0.05.

FIGS. 14A and 14B are bar graphs showing IL1-β gene expression at day 0, week 2, and week 4 of BrachT transfected groups normalized to SHAM for non-degenerate (ND, FIG. 14A) and painful-degeneration (PD, FIG. 14B) groups. *p<0.05.

FIGS. 15A and 15B are bar graphs showing IL6 gene expression at day 0, week 2, and week 4 of BrachT transfected groups normalized to SHAM for non-degenerate (ND, FIG. 15A) and painful-degeneration (PD, FIG. 15B) groups. *p<0.05.

FIGS. 16A and 16B are bar graphs showing NGF gene expression at day 0, week 2, and week 4 of BrachT transfected groups normalized to SHAM for non-degenerate (ND, FIG. 16A) and painful-degeneration (PD, FIG. 16B) groups. *p<0.05.

FIGS. 17A and 17B are bar graphs showing GAG normalized to DNA for non-degenerate (FIG. 17A) and painful-degenerate (FIG. 17B) cells for SHAM compared to BrachT transfected groups. *p<0.05, **p<0.005.

FIGS. 18A to 18C show successful EV generation. FIG. 18A shows FOXF1 upregulation in transfected cells. FIG. 18B shows particle count of FOXF1- and PCMV6-loaded EVs. FIG. 18C shows FOXF1 levels in generated EVs.

FIGS. 19A to 19C show successful EV uptake by cells.

FIG. 20 shows EV delivery in in-vivo lumbar disc puncture mouse model with upregulation of healthy markers. FIG. 20 is a bar graph showing gene expression for FOXF1 and Brachyury.

FIG. 21 shows Control (no injury), Injury SHAM, Empty vector injections and FOXF1 injections on Mouse in Vivo showing effects of treatment on mice gripping time indicative of axial strength.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician or veterinarian.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

As used herein, the term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

A “nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,
where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software.

By “specifically hybridizes” is meant that a probe, primer, or oligonucleotide recognizes and physically interacts (that is, base-pairs) with a substantially complementary nucleic acid (for example, a c-met nucleic acid) under high stringency conditions, and does not substantially base pair with other nucleic acids.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 g/ml denatured, sheared carrier DNA such as salmon sperm DNA, followed by washing the hybridization support in 0.1×SSC at approximately 65° C. Other hybridization and wash conditions are well known and are exemplified in Sambrook et al, Molecular Cloning: A Laboratory Manual, Second Edition, Cold Spring Harbor, N.Y. (1989), particularly chapter 11.

### Compositions

Disclosed are polynucleotides comprising nucleic acid sequences encoding transcription factors that can be used to reprogram diseased musculoskeletal cells according to the disclosed methods. Examples of these transcription factors are provided in Tables 1A, 1B, 1C, 1D, 1E, 1F, 1G, and 1H.

TABLE 1A
Notochordal and Nucleus Pulposus (NP) Transcription Factors

| Transcription Factors | Description/Relevance to NP |
|---|---|
| HIF-1α/2α | hypoxia-inducible factor-1α: |
| | “Transactivate many pro-survival genes |
| | in NP; absolutely necessary for postnatal |
| | NP cell survival” |
| | Strong driver of glycolytic metabolism in the |
| | avascular IVD environment |
| | upregulates: GLUT1/3, GAPDH, Aggrecan, B-1, |
| | 3-glucuronyltransferase 1, galectin-3 |
| | Suppresses expression of: ANK (pyrophosphate |
| | transporter) |
| | Mice model of HIF-1 knockout has been shown to |
| | promote cell death with fibrous NP |
| | involved in pathogenesis of OA |
| Shh (Wnt Shh | Sonic hedgehog: |
| signaling) pathways) | “Signaling ligand necessary for postnatal |
| | function of NP cells” |
| | Increases expression of Brachyury T and aggrecan |
| | Member of the hedgehog family including DHH |
| | and IHH |
| T Family | T-box Family transcription factor: |
| (Brachyury, etc) | “Transcription factor necessary for |
| | notochordal morphogenesis and |
| | patterning” |
| PAX1 | Paired Box Protein 1 |
| FOX Family (FOXF1, | Critical to formation of Intervertebral disc NP |
| FOXA1, A2, ect) | |
| SOX Family (SOX9, | Chondrogenic markers: |
| SOX5, SOX6, etc.) | SOX9 shown to increase expression of COLII post |
| | adenoviral transfection in NP tissue. |
| | SOX5 and SOX6 have been shown to be critical |
| | in ECM sheath formation, notochord cell survival, |
| | and nucleus pulposus formation |
| NOTO | Notochord Homeobox |
| | regulates notochord development |
| | (precursor to NP) |

TABLE 1B
Annulus Fibrosus Transcription Factors

| Transcription Factors | Description/Relevance to AF |
|---|---|
| Mohawk Family | Homeobox protein: |
| | Key transcription factor regulating AF |
| | development shown in humans and mice |
| Scleraxis | Part of basic helix-loop-helix (bHLH) super family |
| | required in musculoskeletal tissue maturation such |
| | as AF, ligaments, and tendons. |
| Tenomodulin | highly expressed in AF cells compared to NP |
| | Cells |
| PAX9 | Paired Box gene 9 |

TABLE 1C
Cartilage Endplate/Articular Cartilage Transcription Factors

| Transcription Factors | Description/Relevance to CEP |
|---|---|
| NFAT Family | Nucleated factor of activate T-Cells Family: |
| (Nfat1, ect) | Nfat1 deficiency causes OA |
| C-1-1 | ets transcription factor: |
| | involved in AC development |
| | Note: Runx2 expression has been known to affect |
| | C-1-1 vs versa |
| PGC1α | regulates chondrogenesis with SOX9 |
| Osterix | required for calcification and degradation of |
| | cartilage matrixes (More for bone formation) |
| MEF2C | MEF2C is regulated by SOX9 in positive feedback |
| | loop |
| | Prevents hypertrophy |
| | stabilizes chondrogenic phenotype |
| SOX Family | important for chondrocyte phenotype |
| Nkx3-2 | Maintains sox9 expression |

TABLE 1D
Osteocytes, Osteoclasts, Osteoblast Transcription Factors

| | Description/Relevance to Osteocytes, Osteoclasts, |
|---|---|
| Transcription Factors | Osteoblasts |
| RUNX2 | Runt-related transcription factor 2 for osteoblast |
| | differentiation |
| Foxc1 | Associated with endochondral ossification and |
| | osteoblast differentiation |
| AP1 Complex (Fos, | Responsible for cell proliferation, differentiation, |
| FosB, Fra1, Fra2, Jun, | apoptosis, ect and essential roles bone |
| JunB, JunD) | development |
| Zfp36 | Regulates HSP70 family proteins to protect |
| | against Osteoarthritis |
| Ebf1, Ebf3 | Regulates osteoblast and adipocyte lineages |
| Maf | Promotes age-associated osteoblast differentiation |
| Mef2c | Controls chondrocyte hypertrophy and bone |
| | development |
| Nupr1 | Increase bone volume |
| Twist1/2, Dermo-1 | Malfunctions in these genes cause inhibitory |
| | effects on osteoblast proliferation/differentiation |
| Maged1 | Overexpression in bone |
| Satb2 | Regulates osteoblast differentiation |
| LMP-3 | Induces osteogenic differentiation of fibroblasts |
| Oct3/4, Sox2, Klf4, | Yamanaka factor |
| c-Myc | |
| Osterix (Sp7) | Main Osteoblast-specific transcription factor |
| Dlx3, Dlx5, Dlx6 | Co-activates RUNX2 |
| C/EBPs, ATF4 | Lack of transcription factor in-vivo mice results in |
| | delayed skeletal development and decreased bone |
| | formation |
| NFATc | Bone deficiency in absence of gene and involved |
| | in chondrogenesis |
| Smads | Part of TGF-B/BMP signal transduction pathway |
| Menin | Enhances Runx2 transcriptional activity |
| Msx1, Msx2 | Important roles in skeletal development |
| NF-1 | Osteoclast differentiation |
| Krox20 and SP3 | Involved in endochondral ossification and |
| | osteoblast differentiation |
| Ob-1 | Highly expressed during osteoblast differentiation |

TABLE 1E
Tenocyte and Ligament Transcription Factors

| Transcription | Description/Relevance to Tenocyte/Tendon and |
|---|---|
| Factors | Ligaments |
| Egr1,2 | Tendon development, healing, and differentiation |
| Scleraxis, Mkx | Generation of tendon progenitors |
| Six1/2, Eya1/2 | Muscle transcription factors involved in tendon |
| | regeneration |
| Pea3 | Induces scleraxis expression |
| Mohawk | Crucial for tendon/ligament homeostasis |

TABLE 1F
Synoviocyte Transcription Factors

| Transcription Factors | Description/Relevance to Synoviocytes/Synovium |
|---|---|
| SOX4, 5, 11 | Promotes migration and invasion of synoviocytes |
| NFAT5 | Regulation of proinflammatory genes |
| BCL-6 | Master Transcription factor involved in immunity |
| HIF-1α, 2α | Synoviocyte differentiation |

TABLE 1G
Monocytes and Myofibroblasts

| | Description/Relevance to Monocytes and |
|---|---|
| Transcription Factors | Myofibroblasts |
| MyoD | Myoblast determination protein |
| Myf5 | Key role in regulation of myogenesis |
| Myogenin | Induction of myogenesis |
| PU.1 | Important for macrophage and monocyte |
| | development |
| M-CSFR Pathway | Important for monocyte development |
| Regulatios | |
| C/EBPα | Directs monocyte differentiation |
| SRF | Critical for myofibroblast activation |
| GLI2 | Profibrotic secretion factor |

TABLE 1H
Dorsal Root Ganglion Transcription Factors

| Transcription Factors | Description/Relevance to DRG |
|---|---|
| FoxO | Upregualated in uninjured rodent DRG model |
| Sp4 | Predominantly expressed in neurons |
| ATF2, 3 | Potential regeneration and downregulated in |
| | injured DRG |
| Etv4, Etv5 | Expressed in DRG development |
| Sox11 | Promotes nerve regeneration |
| Rest | Repressor element 1-silencing transcription |
| | factors regulators neuron remodeling |
| Runx1, 3 | Axonal Growth |

The amino acid and nucleic acid sequences encoding Forkhead-box (FOX) family proteins, a Mohawk family proteins, Scleraxis, NFAT Family proteins, C-1-1, PGC1α, Osterix, and MEF2C are known in the art.

In some embodiments, Forkhead box F1 (FOXF1) comprises the amino acid sequence: MSSAPEKQQPPHGGGGGGGGGGGAAMDPASSGPSKAKKTNAGIRRPEKPPYSYIALIVM AIQSSPTKRLTLSEIYQFLQSRFPFFRGSYQGWKNSVRHNLSLNECFIKLPKGLGRPGKGH YWTIDPASEFMFEEGSFRRRPRGFRRKCQALKPMYSMMNGLGFNHLPDTYGFQGSAGGL SCPPNSLALEGGLGMMNGHLPGNVDGMALPSHSVPHLPSNGGHSYMGGCGGAAAGEYP HHDSSVPASPLLPTGAGGVMEPHAVYSGSAAAWPPSASAALNSGASYIKQQPLSPCNPAA NPLSGSLSTHSLEQPYLHQNSHNAPAELQGIPRYHSQSPSMCDRKEFVFSFNAMASSSMH SAGGGSYYHQQVTYQDIKPCVM (SEQ ID NO:1; NP_001442.2), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:1).

In some embodiments, the nucleic acid sequence encoding FOXF1 comprises the nucleic acid sequence: ATGTCTTCGGCGCCCGAGAAGCAGCAGCCACCGCACGGCGGCGGCGGCGGCGGCGG CGGGGGAGGCGGCGCGGCCATGGACCCCGCGTCGTCCGGCCCGTCCAAGGCCAAGA AGACCAACGCCGGCATCCGGCGCCCGGAGAAGCCGCCCTATTCCTACATCGCGCTCA TCGTCATGGCCATCCAGAGTTCACCCACCAAGCGCCTGACGCTGAGCGAGATCTACCA GTTCCTGCAGAGCCGCTTCCCCTTCTTCCGGGGCTCCTACCAGGGCTGGAAGAACTCC GTGCGCCACAACCTCTCGCTCAACGAGTGCTTCATCAAGCTACCCAAGGGCCTTGGGC GGCCCGGCAAGGGCCACTACTGGACCATCGACCCGGCCAGCGAGTTCATGTTCGAGG AGGGCTCCTTTCGGCGGCGGCCGCGCGGCTTCCGAAGGAAATGCCAGGCGCTCAAG CCCATGTACAGCATGATGAACGGGCTCGGCTTCAACCACCTCCCGGACACCTACGGCT TCCAGGGCTCGGCCGGCGGCCTCTCGTGCCCGCCCAACAGCCTGGCGCTGGAGGGC GGCCTGGGCATGATGAACGGCCACTTGCCGGGCAACGTGGACGGCATGGCCCTGCC CAGCCACTCGGTGCCCCACCTGCCTTCCAACGGCGGCCACTCGTACATGGGCGGCTG CGGCGGCGCGGCGGCCGGCGAGTACCCGCACCACGACAGCTCGGTGCCCGCCTCCC CGCTGCTGCCCACCGGCGCCGGTGGGGTCATGGAGCCGCACGCCGTCTACTCGGGC TCGGCGGCGGCCTGGCCGCCCTCGGCGTCCGCGGCGCTCAACAGCGGCGCCTCTTA TATCAAGCAGCAGCCCCTGTCCCCCTGTAACCCCGCGGCCAACCCCCTGTCCGGCAG CCTCTCCACGCACTCCCTGGAGCAGCCGTATCTGCACCAGAACAGCCACAACGCCCCA GCCGAGCTGCAAGGCATCCCGCGGTATCACTCGCAGTCGCCCAGCATGTGTGACCGA AAGGAGTTTGTCTTCTCTTTCAACGCCATGGCGTCCTCTTCCATGCACTCGGCCGGCG GGGGCTCCTACTACCACCAGCAGGTCACCTACCAAGACATCAAGCCTTGCGTGATG (SEQ ID NO:2; NM_001451), or a nucleic acid sequence that hybridizes to a nucleic acid sequence consisting of SEQ ID NO:2 under stringent hybridization conditions.

In some embodiments, Forkhead Box A1 (FOXA1) comprises the amino acid sequence MLGTVKMEGHETSDWNSYYADTQEAYSSVPVSNMNSGLGSMNSMNTYMTMNTMTTSGN MTPASFNMSYANPGLGAGLSPGAVAGMPGGSAGAMNSMTAAGVTAMGTALSPSGMGAM GAQQAASMNGLGPYAAAMNPCMSPMAYAPSNLGRSRAGGGGDAKTFKRSYPHAKPPYS YISLITMAIQQAPSKMLTLSEIYQWIMDLFPYYRQNQQRWQNSIRHSLSFNDCFVKVARSPD KPGKGSYWTLHPDSGNMFENGCYLRRQKRFKCEKQPGAGGGGGSGSGGSGAKGGPES RKDPSGASNPSADSPLHRGVHGKTGQLEGAPAPGPAASPQTLDHSGATATGGASELKTP ASSTAPPISSGPGALASVPASHPAHGLAPHESQLHLKGDPHYSFNHPFSINNLMSSSEQQH KLDFKAYEQALQYSPYGSTLPASLPLGSASVTTRSPIEPSALEPAYYQGVYSRPVLNTS (SEQ ID NO:3; NP_004487), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:3.

In some embodiments, the nucleic acid sequence encoding FOXA1 comprises the nucleic acid sequence ATGTTAGGAACTGTGAAGATGGAAGGGCATGAAACCAGCGACTGGAACAGCTACTACG CAGACACGCAGGAGGCCTACTCCTCCGTCCCGGTCAGCAACATGAACTCAGGCCTGG GCTCCATGAACTCCATGAACACCTACATGACCATGAACACCATGACTACGAGCGGCAAC ATGACCCCGGCGTCCTTCAACATGTCCTATGCCAACCCGGGCCTAGGGGCCGGCCTGA GTCCCGGCGCAGTAGCCGGCATGCCGGGGGGCTCGGCGGGCGCCATGAACAGCATG ACTGCGGCCGGCGTGACGGCCATGGGTACGGCGCTGAGCCCGAGCGGCATGGGCGC CATGGGTGCGCAGCAGGCGGCCTCCATGAATGGCCTGGGCCCCTACGCGGCCGCCAT GAACCCGTGCATGAGCCCCATGGCGTACGCGCCGTCCAACCTGGGCCGCAGCCGCGC GGGCGGCGGCGGCGACGCCAAGACGTTCAAGCGCAGCTACCCGCACGCCAAGCCGC CCTACTCGTACATCTCGCTCATCACCATGGCCATCCAGCAGGCGCCCAGCAAGATGCTC ACGCTGAGCGAGATCTACCAGTGGATCATGGACCTCTTCCCCTATTACCGGCAGAACCA GCAGCGCTGGCAGAACTCCATCCGCCACTCGCTGTCCTTCAATGACTGCTTCGTCAAG GTGGCACGCTCCCCGGACAAGCCGGGCAAGGGCTCCTACTGGACGCTGCACCCGGA CTCCGGCAACATGTTCGAGAACGGCTGCTACTTGCGCCGCCAGAAGCGCTTCAAGTGC GAGAAGCAGCCGGGGGCCGGCGGCGGGGGCGGGAGCGGAAGCGGGGGCAGCGGC GCCAAGGGCGGCCCTGAGAGCCGCAAGGACCCCTCTGGCGCCTCTAACCCCAGCGC CGACTCGCCCCTCCATCGGGGTGTGCACGGGAAGACCGGCCAGCTAGAGGGCGCGC CGGCCCCCGGGCCCGCCGCCAGCCCCCAGACTCTGGACCACAGTGGGGCGACGGCG ACAGGGGGCGCCTCGGAGTTGAAGACTCCAGCCTCCTCAACTGCGCCCCCCATAAGCT CCGGGCCCGGGGCGCTGGCCTCTGTGCCCGCCTCTCACCCGGCACACGGCTTGGCA CCCCACGAGTCCCAGCTGCACCTGAAAGGGGACCCCCACTACTCCTTCAACCACCCGT TCTCCATCAACAACCTCATGTCCTCCTCGGAGCAGCAGCATAAGCTGGACTTCAAGGCA TACGAACAGGCACTGCAATACTCGCCTTACGGCTCTACGTTGCCCGCCAGCCTGCCTCT AGGCAGCGCCTCGGTGACCACCAGGAGCCCCATCGAGCCCTCAGCCCTGGAGCCGG CGTACTACCAAGGTGTGTATTCCAGACCCGTCCTAAACACTTCC (SEQ ID NO:4; NM_004496), or a nucleic acid sequence that hybridizes to a nucleic acid sequence consisting of SEQ ID NO:4 under stringent hybridization conditions.

In some embodiments, Forkhead box A2 (FOXA2) comprises the amino acid sequence MLGAVKMEGHEPSDWSSYYAEPEGYSSVSNMNAGLGMNGMNTYMSMSAAAMGSGSGN MSAGSMNMSSYVGAGMSPSLAGMSPGAGAMAGMGGSAGAAGVAGMGPHLSPSLSPLG GQAAGAMGGLAPYANMNSMSPMYGQAGLSRARDPKTYRRSYTHAKPPYSYISLITMAIQQ SPNKMLTLSEIYQWIMDLFPFYRQNQQRWQNSIRHSLSFNDCFLKVPRSPDKPGKGSFWT LHPDSGNMFENGCYLRRQKRFKCEKQLALKEAAGAAGSGKKAAAGAQASQAQLGEAAGP ASETPAGTESPHSSASPCQEHKRGGLGELKGTPAAALSPPEPAPSPGQQQQAAAHLLGPP HHPGLPPEAHLKPEHHYAFNHPFSINNLMSSEQQHHHSHHHHQPHKMDLKAYEQVMHYP GYGSPMPGSLAMGPVTNKTGLDASPLAADTSYYQGVYSRPIMNSS (SEQ ID NO:5; NP_710141), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:5.

In some embodiments, the nucleic acid sequence encoding FOXA2 comprises the nucleic acid sequence: ATGCTGGGAGCGGTGAAGATGGAAGGGCACGAGCCGTCCGACTGGAGCAGCTACTAT GCAGAGCCCGAGGGCTACTCCTCCGTGAGCAACATGAACGCCGGCCTGGGGATGAAC GGCATGAACACGTACATGAGCATGTCGGCGGCCGCCATGGGCAGCGGCTCGGGCAAC ATGAGCGCGGGCTCCATGAACATGTCGTCGTACGTGGGCGCTGGCATGAGCCCGTCC CTGGCGGGGATGTCCCCCGGCGCGGGCGCCATGGCGGGCATGGGCGGCTCGGCCG GGGCGGCTGGCGTGGCGGGCATGGGGCCGCACTTGAGTCCCAGCCTGAGCCCGCTC GGGGGGCAGGCGGCCGGGGCCATGGGCGGCCTGGCCCCCTACGCCAACATGAACTC CATGAGCCCCATGTACGGGCAGGCGGGCCTGAGCCGCGCCCGCGACCCCAAGACCT ACAGGCGCAGCTACACGCACGCAAAGCCGCCCTACTCGTACATCTCGCTCATCACCAT GGCCATCCAGCAGAGCCCCAACAAGATGCTGACGCTGAGCGAGATCTACCAGTGGAT CATGGACCTCTTCCCCTTCTACCGGCAGAACCAGCAGCGCTGGCAGAACTCCATCCGC CACTCGCTCTCCTTCAACGACTGTTTCCTGAAGGTGCCCCGCTCGCCCGACAAGCCCG GCAAGGGCTCCTTCTGGACCCTGCACCCTGACTCGGGCAACATGTTCGAGAACGGCT GCTACCTGCGCCGCCAGAAGCGCTTCAAGTGCGAGAAGCAGCTGGCGCTGAAGGAGG CCGCAGGCGCCGCCGGCAGCGGCAAGAAGGCGGCCGCCGGGGCCCAGGCCTCACA GGCTCAACTCGGGGAGGCCGCCGGGCCGGCCTCCGAGACTCCGGCGGGCACCGAGT CGCCTCACTCGAGCGCCTCCCCGTGCCAGGAGCACAAGCGAGGGGGCCTGGGAGAG CTGAAGGGGACGCCGGCTGCGGCGCTGAGCCCCCCAGAGCCGGCGCCCTCTCCCGG GCAGCAGCAGCAGGCCGCGGCCCACCTGCTGGGCCCGCCCCACCACCCGGGCCTGC CGCCTGAGGCCCACCTGAAGCCGGAACACCACTACGCCTTCAACCACCCGTTCTCCAT CAACAACCTCATGTCCTCGGAGCAGCAGCACCACCACAGCCACCACCACCACCAGCC CCACAAAATGGACCTCAAGGCCTACGAACAGGTGATGCACTACCCCGGCTACGGTTCC CCCATGCCTGGCAGCTTGGCCATGGGCCCGGTCACGAACAAAACGGGCCTGGACGCC TCGCCCCTGGCCGCAGATACCTCCTACTACCAGGGGGTGTACTCCCGGCCCATTATGA ACTCCTCT (SEQ ID NO:6; NM_153675), or a nucleic acid sequence that hybridizes to a nucleic acid sequence consisting of SEQ ID NO:6 under stringent hybridization conditions.

In some embodiments, Forkhead box A3 (FOXA3) comprises the amino acid sequence: MLGSVKMEAHDLAEWSYYPEAGEVYSPVTPVPTMAPLNSYMTLNPLSSPYPPGGLPASPL PSGPLAPPAPAAPLGPTFPGLGVSGGSSSSGYGAPGPGLVHGKEMPKGYRRPLAHAKPP YSYISLITMAIQQAPGKMLTLSEIYQWIMDLFPYYRENQQRWQNSIRHSLSFNDCFVKVARS PDKPGKGSYWALHPSSGNMFENGCYLRRQKRFKLEEKVKKGGSGAATTTRNGTGSAAST TTPAATVTSPPQPPPPAPEPEAQGGEDVGALDCGSPASSTPYFTGLELPGELKLDAPYNFN HPFSINNLMSEQTPAPPKLDVGFGGYGAEGGEPGVYYQGLYSRSLLNAS (SEQ ID NO:7; NP_004488), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:7.

In some embodiments, the nucleic acid sequence encoding FOXA3 comprises the nucleic acid sequence: ATGCTGGGCTCAGTGAAGATGGAGGCCCATGACCTGGCCGAGTGGAGCTACTACCCGG AGGCGGGCGAGGTCTACTCGCCGGTGACCCCAGTGCCCACCATGGCCCCCCTCAACT CCTACATGACCCTGAATCCTCTAAGCTCTCCCTATCCCCCTGGGGGGCTCCCTGCCTCC CCACTGCCCTCAGGACCCCTGGCACCCCCAGCACCTGCAGCCCCCCTGGGGCCCACT TTCCCAGGCCTGGGTGTCAGCGGTGGCAGCAGCAGCTCCGGGTACGGGGCCCCGGG TCCTGGGCTGGTGCACGGGAAGGAGATGCCGAAGGGGTATCGGCGGCCCCTGGCACA CGCCAAGCCACCGTATTCCTATATCTCACTCATCACCATGGCCATCCAGCAGGCGCCGG GCAAGATGCTGACCTTGAGTGAAATCTACCAGTGGATCATGGACCTCTTCCCTTACTACC GGGAGAATCAGCAGCGCTGGCAGAACTCCATTCGCCACTCGCTGTCTTTCAACGACTG CTTCGTCAAGGTGGCGCGTTCCCCAGACAAGCCTGGCAAGGGCTCCTACTGGGCCCTA CACCCCAGCTCAGGGAACATGTTTGAGAATGGCTGCTACCTGCGCCGCCAGAAACGCT TCAAGCTGGAGGAGAAGGTGAAAAAAGGGGGCAGCGGGGCTGCCACCACCACCAGG AACGGGACAGGGTCTGCTGCCTCGACCACCACCCCCGCGGCCACAGTCACCTCCCCG CCCCAGCCCCCGCCTCCAGCCCCTGAGCCTGAGGCCCAGGGCGGGGAAGATGTGGG GGCTCTGGACTGTGGCTCACCCGCTTCCTCCACACCCTATTTCACTGGCCTGGAGCTC CCAGGGGAGCTGAAGCTGGACGCGCCCTACAACTTCAACCACCCTTTCTCCATCAACA ACCTAATGTCAGAACAGACACCAGCACCTCCCAAACTGGACGTGGGGTTTGGGGGCTA CGGGGCTGAAGGTGGGGAGCCTGGAGTCTACTACCAGGGCCTCTATTCCCGCTCTTTG CTTAATGCATCC (SEQ ID NO:8; NM_004497), or a nucleic acid sequence that hybridizes to a nucleic acid sequence consisting of SEQ ID NO:8 under stringent hybridization conditions.

In some embodiments, Forkhead box E1 (FOXB1) comprises the amino acid sequence: MPRPGRNTYSDQKPPYSYISLTAMAIQSSPEKMLPLSEIYKFIMDRFPYYRENTQRWQNSL RHNLSFNDCFIKIPRRPDQPGKGSFWALHPSCGDMFENGSFLRRRKRFKVLKSDHLAPSK PADAAQYLQQQAKLRLSALAASGTHLPQMPAAAYNLGGVAQPSGFKHPFAIENIIAREYKM PGGLAFSAMQPVPAAYPLPNQLTTMGSSLGTGWPHVYGSAGMIDSATPISMASGDYSAYG VPLKPLCHAAGQTLPAIPVPIKPTPAAVPALPALPAPIPTLLSNSPPSLSPTSSQTATSQSSPA TPSETLTSPASALHSVAVH (SEQ ID NO:9; NP_036314), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:9.

In some embodiments, the nucleic acid sequence encoding FOXB1 comprises the nucleic acid sequence: ATGCCTCGGCCCGGCCGCAACACGTACAGCGACCAGAAGCCGCCCTACTCGTACATCT CGCTGACCGCTATGGCCATCCAGAGCTCTCCCGAGAAGATGCTGCCGCTGAGCGAGAT CTACAAGTTCATCATGGACCGCTTCCCCTACTACAGGGAGAACACGCAGCGCTGGCAG AACAGTCTGCGCCACAACCTCTCCTTCAACGACTGCTTCATCAAGATCCCGCGGCGGC CGGACCAGCCAGGCAAGGGCAGCTTCTGGGCGCTGCACCCAAGCTGCGGGGACATGT TCGAGAACGGCAGCTTCCTGCGGCGCCGCAAGCGCTTCAAGGTGCTTAAGTCCGACC ACCTGGCGCCCAGCAAGCCAGCCGACGCGGCGCAGTACCTGCAGCAGCAGGCCAAG CTGCGGCTCAGCGCGCTGGCGGCCTCGGGCACGCACCTGCCACAGATGCCCGCCGC CGCCTACAACTTGGGCGGCGTGGCGCAGCCCTCGGGCTTCAAGCACCCCTTCGCCAT CGAGAACATCATCGCGCGGGAATACAAGATGCCTGGGGGGCTGGCCTTCTCCGCCATG CAGCCGGTGCCCGCTGCCTACCCGCTCCCCAACCAGTTGACTACCATGGGCAGCTCG CTGGGCACCGGCTGGCCACACGTGTATGGCTCCGCCGGCATGATCGACTCGGCCACC CCCATCTCCATGGCGAGTGGCGACTACAGCGCCTACGGCGTGCCGTTGAAGCCGCTGT GCCACGCGGCGGGCCAAACGCTGCCCGCCATCCCCGTGCCCATTAAGCCCACGCCGG CCGCCGTGCCCGCGCTGCCTGCGCTGCCAGCGCCCATCCCCACCTTGCTCTCGAACT CGCCGCCCTCGCTCAGCCCCACGTCCTCGCAAACAGCCACCAGCCAAAGCAGCCCCG CCACCCCCAGCGAAACGCTCACCAGCCCGGCCTCCGCCTTGCACTCGGTGGCGGTGC AC (SEQ ID NO:10; NM_012182), or a nucleic acid sequence that hybridizes to a nucleic acid sequence consisting of SEQ ID NO:10 under stringent hybridization conditions.

In some embodiments, Forkhead box B2 (FOXB2) comprises the amino acid sequence: MPRPGKSSYSDQKPPYSYISLTAMAIQHSAEKMLPLSDIYKFIMERFPYYREHTQRWQNSL RHNLSFNDCFIKIPRRPDQPGKGSFWALHPDCGDMFENGSFLRRRKRFKVLRADHTHLHA GSTKSAPGAGPGGHLHPHHHHHPHHHHHHHAAAHHHHHHHPPQPPPPPPPPPPHMVHY FHQQPPTAPQPPPHLPSQPPQQPPQQSQPQQPSHPGKMQEAAAVAAAAAAAAAAAVGS VGRLSQFPPYGLGSAAAAAAAAAASTSGFKHPFAIENIIGRDYKGVLQAGGLPLASVMHHL GYPVPGQLGNVVSSVWPHVGVMDSVAAAAAAAAAAGVPVGPEYGAFGVPVKSLCHSASQ SLPAMPVPIKPTPALPPVSALQPGLTVPAASQQPPAPSTVCSAAAASPVASLLEPTAPTSAE SKGGSLHSVLVHS (SEQ ID NO:11; NP_001013757), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:11.

In some embodiments, the nucleic acid sequence encoding FOXB2 comprises the nucleic acid sequence ATGCCTCGGCCCGGCAAGTCATCTTATTCTGATCAAAAGCCACCCTACTCATATATTAG CCTCACAGCGATGGCTATACAGCATTCAGCTGAGAAGATGTTGCCTCTCTCCGACATCT ACAAATCATCATGGAGCGGTTCCCCTACTACCGCGAACACACCCAGCGGTGGCAGAAC TCACTTAGACACAACCTGAGCTTCAATGATTGTTTTATTAAGATTCCCAGGAGGCCGGA CCAGCCAGGCAAGGGTTCATTCTGGGCACTCCACCCCGATTGCGGAGACATGTTTGAA AACGGGAGCTTTCTCCGACGACGGAAGAGATTTAAGGTCCTGAGAGCCGATCATACCC ATCTCCACGCCGGGTCCACTAAATCTGCACCGGGGGCCGGCCCAGGCGGGCATCTCC ATCCCCACCACCACCATCACCCCCATCACCATCATCATCACCACGCCGCTGCACACCA CCACCATCACCACCACCCCCCACAACCACCCCCTCCCCCGCCACCCCCGCCACCCCA CATGGTCCACTACTTTCACCAACAGCCCCCCACCGCCCCGCAGCCCCCGCCCCACCT GCCATCACAGCCCCCCCAGCAGCCCCCACAGCAAAGCCAGCCCCAGCAACCTAGCCA TCCTGGTAAAATGCAGGAGGCTGCGGCGGTGGCTGCGGCTGCAGCTGCCGCTGCTGC TGCGGCTGTTGGGTCTGTGGGCAGACTGAGCCAGTTCCCTCCCTACGGCTTGGGTTC CGCCGCCGCGGCGGCCGCCGCCGCTGCAGCCAGCACTTCCGGCTTTAAGCATCCATT TGCTATTGAGAACATCATTGGCCGCGACTATAAAGGCGTCCTCCAAGCCGGAGGACTC CCACTCGCGAGTGTGATGCATCACTTGGGCTATCCAGTGCCAGGCCAGCTGGGTAAC GTCGTGTCCTCCGTCTGGCCCCACGTGGGGGTAATGGACAGTGTGGCAGCAGCCGCT GCCGCTGCAGCTGCCGCTGGCGTTCCAGTAGGTCCCGAATATGGAGCATTCGGCGTG CCCGTGAAGTCCCTGTGCCACTCTGCAAGCCAGAGCCTGCCAGCCATGCCGGTGCCC ATCAAGCCAACACCAGCCCTCCCACCAGTGTCTGCCTTGCAGCCAGGACTCACGGTGC CCGCCGCATCTCAGCAGCCTCCAGCACCCTCAACGGTGTGCAGCGCCGCAGCCGCTA GCCCCGTGGCCAGCCTCCTGGAACCCACTGCACCCACATCAGCTGAGTCAAAAGGTG GAAGCCTTCATTCCGTGTTGGTGCACTCA (SEQ ID NO:12; NM_001013735), or a nucleic acid sequence that hybridizes to a nucleic acid sequence consisting of SEQ ID NO:12 under stringent hybridization conditions.

In some embodiments, Forkhead box C1 (FOXC1) comprises the amino acid sequence: MQARYSVSSPNSLGVVPYLGGEQSYYRAAAAAAGGGYTAMPAPMSVYSHPAHAEQYPG GMARAYGPYTPQPQPKDMVKPPYSYIALITMAIQNAPDKKITLNGIYQFIMDRFPFYRDNKQ GWQNSIRHNLSLNECFVKVPRDDKKPGKGSYWTLDPDSYNMFENGSFLRRRRRFKKKDA VKDKEEKDRLHLKEPPPPGRQPPPAPPEQADGNAPGPQPPPVRIQDIKTENGTCPSPPQP LSPAAALGSGSAAAVPKIESPDSSSSSLSSGSSPPGSLPSARPLSLDGADSAPPPPAPSAP PPHHSQGFSVDNIMTSLRGSPQSAAAELSSGLLASAAASSRAGIAPPLALGAYSPGQSSLY SSPCSQTSSAGSSGGGGGGAGAAGGAGGAGTYHCNLQAMSLYAAGERGGHLQGAPGG AGGSAVDDPLPDYSLPPVTSSSSSSLSHGGGGGGGGGGQEAGHHPAAHQGRLTSWYLN QAGGDLGHLASAAAAAAAAGYPGQQQNFHSVREMFESQRIGLNNSPVNGNSSCQMAFPS SQSLYRTSGAFVYDCSKF (SEQ ID NO:13; NP_001444), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:13.

In some embodiments, the nucleic acid sequence encoding FOXC1 comprises the nucleic acid sequence: ATGCAGGCGCGCTACTCCGTGTCCAGCCCCAACTCCCTGGGAGTGGTGCCC TACCTCGGCGGCGAGCAGAGCTACTACCGCGCGGCGGCCGCGGCGGCCGGGGGCG GCTACACCGCCATGCCGGCCCCCATGAGCGTGTACTCGCACCCTGCGCACGCCGAGC AGTACCCGGGCGGCATGGCCCGCGCCTACGGGCCCTACACGCCGCAGCCGCAGCCC AAGGACATGGTGAAGCCGCCCTATAGCTACATCGCGCTCATCACCATGGCCATCCAGA ACGCCCCGGACAAGAAGATCACCCTGAACGGCATCTACCAGTTCATCATGGACCGCTT CCCCTTCTACCGGGACAACAAGCAGGGCTGGCAGAACAGCATCCGCCACAACCTCTC GCTCAACGAGTGCTTCGTCAAGGTGCCGCGCGACGACAAGAAGCCGGGCAAGGGCAG CTACTGGACGCTGGACCCGGACTCCTACAACATGTTCGAGAACGGCAGCTTCCTGCGG CGGCGGCGGCGCTTCAAGAAGAAGGACGCGGTGAAGGACAAGGAGGAGAAGGACAG GCTGCACCTCAAGGAGCCGCCCCCGCCCGGCCGCCAGCCCCCGCCCGCGCCGCCGG AGCAGGCCGACGGCAACGCGCCCGGTCCGCAGCCGCCGCCCGTGCGCATCCAGGAC ATCAAGACCGAGAACGGTACGTGCCCCTCGCCGCCCCAGCCCCTGTCCCCGGCCGCC GCCCTGGGCAGCGGCAGCGCCGCCGCGGTGCCCAAGATCGAGAGCCCCGACAGCAG CAGCAGCAGCCTGTCCAGCGGGAGCAGCCCCCCGGGCAGCCTGCCGTCGGCGCGGC CGCTCAGCCTGGACGGTGCGGATTCCGCGCCGCCGCCGCCCGCGCCCTCCGCCCCG CCGCCGCACCATAGCCAGGGCTTCAGCGTGGACAACATCATGACGTCGCTGCGGGGG TCGCCGCAGAGCGCGGCCGCGGAGCTCAGCTCCGGCCTTCTGGCCTCGGCGGCCGC GTCCTCGCGCGCGGGGATCGCACCCCCGCTGGCGCTCGGCGCCTACTCGCCCGGCC AGAGCTCCCTCTACAGCTCCCCCTGCAGCCAGACCTCCAGCGCGGGCAGCTCGGGCG GCGGCGGCGGCGGCGCGGGGGCCGCGGGGGGCGCGGGCGGCGCCGGGACCTACC ACTGCAACCTGCAAGCCATGAGCCTGTACGCGGCCGGCGAGCGCGGGGGCCACTTG CAGGGCGCGCCCGGGGGCGCGGGCGGCTCGGCCGTGGACGACCCCCTGCCCGACT ACTCTCTGCCTCCGGTCACCAGCAGCAGCTCGTCGTCCCTGAGTCACGGCGGCGGCG GCGGCGGCGGCGGGGGAGGCCAGGAGGCCGGCCACCACCCTGCGGCCCACCAAGG CCGCCTCACCTCGTGGTACCTGAACCAGGCGGGCGGAGACCTGGGCCACTTGGCGAG CGCGGCGGCGGCGGCGGCGGCCGCAGGCTACCCGGGCCAGCAGCAGAACTTCCACT CGGTGCGGGAGATGTTCGAGTCACAGAGGATCGGCTTGAACAACTCTCCAGTGAACG GGAATAGTAGCTGTCAAATGGCCTTCCCTTCCAGCCAGTCTCTGTACCGCACGTCCGG AGCTTTCGTCTACGACTGTAGCAAGTTT (SEQ ID NO:14; NM_001453), or a nucleic acid sequence that hybridizes to a nucleic acid sequence consisting of SEQ ID NO:14 under stringent hybridization conditions.

In some embodiments, Forkhead box C2 (FOXC2) comprises the amino acid sequence MQARYSVSDPNALGVVPYLSEQNYYRAAGSYGGMASPMGVYSGHPEQYSAGMGRSYAP YHHHQPAAPKDLVKPPYSYIALITMAIQNAPEKKITLNGIYQFIMDRFPFYRENKQGWQNSIR HNLSLNECFVKVPRDDKKPGKGSYWTLDPDSYNMFENGSFLRRRRRFKKKDVSKEKEER AHLKEPPPAASKGAPATPHLADAPKEAEKKVVIKSEAASPALPVITKVETLSPESALQGSPR SAASTPAGSPDGSLPEHHAAAPNGLPGFSVENIMTLRTSPPGGELSPGAGRAGLVVPPLAL PYAAAPPAAYGQPCAQGLEAGAAGGYQCSMRAMSLYTGAERPAHMCVPPALDEALSDHP SGPTSPLSALNLAAGQEGALAATGHHHQHHGHHHPQAPPPPPAPQPQPTPQPGAAAAQA ASWYLNHSGDLNHLPGHTFAAQQQTFPNVREMFNSHRLGIENSTLGESQVSGNASCQLP YRSTPPLYRHAAPYSYDCTKY (SEQ ID NO:15; NP_005242), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:15.

In some embodiments, the nucleic acid sequence encoding FOXC2 comprises the nucleic acid sequence: ATGCAGGCGCGCTACTCCGTGTCCGACCCCAACGCCCTGGGAGTGGTGCC CTACCTGAGCGAGCAGAATTACTACCGGGCTGCGGGCAGCTACGGCGGCATGGCCAG CCCCATGGGCGTCTATTCCGGCCACCCGGAGCAGTACAGCGCGGGGATGGGCCGCT CCTACGCGCCCTACCACCACCACCAGCCCGCGGCGCCTAAGGACCTGGTGAAGCCGC CCTACAGCTACATCGCGCTCATCACCATGGCCATCCAGAACGCGCCCGAGAAGAAGAT CACCTTGAACGGCATCTACCAGTTCATCATGGACCGCTTCCCCTTCTACCGGGAGAAC AAGCAGGGCTGGCAGAACAGCATCCGCCACAACCTCTCGCTCAACGAGTGCTTCGTCA AGGTGCCCCGCGACGACAAGAAGCCCGGCAAGGGCAGTTACTGGACCCTGGACCCG GACTCCTACAACATGTTCGAGAACGGCAGCTTCCTGCGGCGCCGGCGGCGCTTCAAA AAGAAGGACGTGTCCAAGGAGAAGGAGGAGCGGGCCCACCTCAAGGAGCCGCCCCC GGCGGCGTCCAAGGGCGCCCCGGCCACCCCCCACCTAGCGGACGCCCCCAAGGAGG CCGAGAAGAAGGTGGTGATCAAGAGCGAGGCGGCGTCCCCGGCGCTGCCGGTCATC ACCAAGGTGGAGACGCTGAGCCCCGAGAGCGCGCTGCAGGGCAGCCCGCGCAGCGC GGCCTCCACGCCCGCCGGCTCCCCCGACGGCTCGCTGCCGGAGCACCACGCCGCGG CGCCCAACGGGCTGCCTGGCTTCAGCGTGGAGAACATCATGACCCTGCGAACGTCGC CGCCGGGCGGAGAGCTGAGCCCGGGGGCCGGACGCGCGGGCCTGGTGGTGCCGCC GCTGGCGCTGCCCTACGCCGCCGCGCCGCCCGCCGCCTACGGCCAGCCGTGCGCTC AGGGCCTGGAGGCCGGGGCCGCCGGGGGCTACCAGTGCAGCATGCGAGCGATGAGC CTGTACACCGGGGCCGAGCGGCCGGCGCACATGTGCGTCCCGCCCGCCCTGGACGA GGCCCTCTCGGACCACCCGAGCGGCCCCACGTCGCCCCTGAGCGCTCTCAACCTCGC CGCCGGCCAGGAGGGCGCGCTCGCCGCCACGGGCCACCACCACCAGCACCACGGCC ACCACCACCCGCAGGCGCCGCCGCCCCCGCCGGCTCCCCAGCCCCAGCCGACGCCG CAGCCCGGGGCCGCCGCGGCGCAGGCGGCCTCCTGGTATCTCAACCACAGCGGGGA CCTGAACCACCTCCCCGGCCACACGTTCGCGGCCCAGCAGCAAACTTTCCCCAACGT GCGGGAGATGTTCAACTCCCACCGGCTGGGGATTGAGAACTCGACCCTCGGGGAGTC CCAGGTGAGTGGCAATGCCAGCTGCCAGCTGCCCTACAGATCCACGCCGCCTCTCTAT CGCCACGCAGCCCCCTACTCCTACGACTGCACGAAATAC (SEQ ID NO:16; NM_005251), or a nucleic acid sequence that hybridizes to a nucleic acid sequence consisting of SEQ ID NO:16 under stringent hybridization conditions.

In some embodiments, Forkhead box D1 (FOXD1) comprises the amino acid sequence: MTLSTEMSDASGLAEETDIDVVGEGEDEEDEEEEDDDEGGGGGPRLAVPAQRRRRRRSY AGEDELEDLEEEEDDDDILLAPPAGGSPAPPGPAPAAGAGAGGGGGGGGAGGGGSAGS GAKNPLVKPPYSYIALITMAILQSPKKRLTLSEICEFISGRFPYYREKFPAWQNSIRHNLSLND CFVKIPREPGNPGKGNYWTLDPESADMFDNGSFLRRRKRFKRQPLLPPNAAAAESLLLRG AGAAGGAGDPAAAAALFPPAPPPPPHAYGYGPYGCGYGLQLPPYAPPSALFAAAAAAAAA AAFHPHSPPPPPPPHGAAAELARTAFGYRPHPLGAALPGPLPASAAKAGGPGASALARSP FSIESIIGGSLGPAAAAAAAAQAAAAAQASPSPSPVAAPPAPGSSGGGCAAQAAVGPAAAL TRSLVAAAAAAASSVSSSAALGTLHQGTALSSVENFTARISNC (SEQ ID NO:17; NP_004463), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:17.

In some embodiments, the nucleic acid sequence encoding FOXD1 comprises the nucleic acid sequence: ATGACCCTGAGCACTGAGATGTCCGATGCCTCTGGCCTCGCCGAGGAAACA GACATCGACGTGGTGGGGGAGGGCGAGGACGAAGAAGACGAGGAAGAGGAGGACGA CGACGAGGGCGGCGGTGGCGGGCCCCGGCTGGCTGTCCCCGCGCAGCGGCGGCGG CGGCGGCGCTCGTACGCCGGGGAGGACGAGCTGGAGGATCTGGAGGAGGAGGAGGA CGACGATGACATCCTGCTGGCCCCGCCTGCTGGGGGCTCCCCGGCGCCCCCGGGCC CGGCCCCGGCGGCGGGGGCAGGAGCCGGTGGGGGCGGCGGCGGCGGCGGCGCGG GCGGCGGCGGGAGCGCGGGTAGCGGCGCCAAGAACCCGCTGGTGAAGCCGCCCTAC TCGTATATCGCGCTCATCACTATGGCCATCCTGCAGAGCCCCAAGAAGCGGCTGACGC TGAGCGAGATCTGTGAGTTCATCAGCGGCCGCTTCCCCTACTACCGGGAGAAGTTCCC CGCCTGGCAGAACAGCATCCGCCACAACCTCTCGCTCAACGACTGCTTCGTCAAGATC CCCCGCGAGCCCGGCAACCCGGGCAAGGGCAACTACTGGACGCTGGACCCGGAGTC CGCCGACATGTTCGACAACGGCAGCTTCCTGCGCCGGAGGAAGCGCTTCAAGCGGCA GCCGCTGCTCCCACCCAACGCCGCGGCCGCCGAGTCTCTGCTGCTGCGCGGCGCGG GAGCCGCAGGGGGCGCGGGCGACCCGGCAGCCGCCGCCGCGCTCTTCCCGCCCGC GCCCCCGCCGCCCCCGCATGCCTACGGCTACGGCCCCTACGGCTGCGGCTACGGCC TGCAGCTGCCGCCTTACGCGCCGCCCTCGGCCCTCTTCGCCGCCGCAGCGGCCGCC GCCGCCGCCGCCGCCTTCCACCCGCACTCGCCCCCGCCGCCCCCGCCACCGCACGG CGCGGCCGCCGAGCTGGCCCGGACCGCCTTCGGCTACCGGCCGCACCCGCTCGGCG CCGCCCTACCCGGCCCCCTGCCGGCCTCCGCGGCCAAGGCGGGCGGCCCGGGCGC CTCAGCGCTGGCGCGCTCGCCCTTCTCCATCGAGAGCATCATCGGGGGCAGCTTGGG CCCGGCCGCCGCTGCCGCCGCCGCCGCGCAGGCCGCCGCCGCCGCTCAGGCCTCG CCCTCGCCCTCGCCGGTGGCGGCGCCGCCAGCTCCCGGATCCAGCGGAGGAGGCTG CGCGGCGCAGGCGGCCGTGGGCCCGGCGGCCGCGCTCACCCGATCCCTCGTGGCC GCCGCGGCCGCCGCCGCCTCCTCAGTCTCCTCGTCCGCCGCCTTGGGGACTCTGCAC CAAGGGACTGCCCTGTCCAGTGTCGAGAACTTTACTGCTAGGATTTCCAATTGT (SEQ ID NO:18; NM_004472), or a nucleic acid sequence that hybridizes to a nucleic acid sequence consisting of SEQ ID NO:18 under stringent hybridization conditions.

In some embodiments, Forkhead box D2 (FOXD2) comprises the amino acid sequence: MTLGSCCCEIMSSESSPAALSEADADIDVVGGGSGGGELPARSGPRAPRDVLPHGHEPPA EEAEADLAEDEEESGGCSDGEPRALASRGAAAAAGSPGPGAAAARGAAGPGPGPPSGG AATRSPLVKPPYSYIALITMAILQSPKKRLTLSEICEFISGRFPYYREKFPAWQNSIRHNLSLN DCFVKIPREPGNPGKGNYWTLDPESADMFDNGSFLRRRKRFKRQPLPPPHPHPHPHPELL LRGGAAAAGDPGAFLPGFAAYGAYGYGYGLALPAYGAPPPGPAPHPHPHPHAFAFAAAAA AAPCQLSVPPGRAAAPPPGPPTASVFAGAGSAPAPAPASGSGPGPGPAGLPAFLGAELGC AKAFYPASLSPPAAGTAAGLPTALLRQGLKTDAGGGAGGGGAGAGQRPSFSIDHIMGHGG GGAAPPGAGEGSPGPPFAAAAGPGGQAQVLAMLTAPALAPVAGHIRLSHPGDALLSSGSR FASKVAGLSGCHF (SEQ ID NO:19; NP_004465), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:19.

In some embodiments, the nucleic acid sequence encoding FOXD2 comprises the nucleic acid sequence: ATGACCCTGGGCAGCTGCTGCTGCGAGATCATGTCCTCCGAGAGCTCCCCG GCCGCGCTGTCCGAGGCCGACGCAGACATAGACGTGGTGGGCGGCGGCAGCGGCGG GGGGGAGCTCCCAGCTCGCTCCGGGCCCCGCGCCCCCCGGGACGTGCTCCCCCACG GCCACGAGCCTCCCGCGGAGGAAGCCGAGGCAGACTTAGCCGAGGACGAGGAGGAG TCTGGTGGCTGCTCGGACGGCGAGCCCCGCGCTCTGGCGTCCCGGGGGGCGGCGGC CGCAGCGGGGAGCCCGGGGCCAGGCGCCGCGGCGGCCCGCGGCGCAGCGGGGCC CGGGCCGGGACCGCCGTCGGGGGGCGCGGCGACGCGGAGCCCGCTGGTGAAGCCG CCCTACTCGTACATCGCGCTCATCACCATGGCCATCCTGCAGAGCCCCAAGAAGCGGC TGACGTTGAGCGAGATCTGCGAGTTCATCAGCGGCCGCTTCCCCTACTACCGGGAGAA GTTCCCCGCCTGGCAGAACAGCATCCGCCACAACCTCTCTCTCAACGACTGCTTCGTC AAGATCCCCCGCGAGCCGGGCAACCCGGGCAAGGGCAACTACTGGACGCTGGACCC GGAGTCGGCCGACATGTTCGACAACGGCAGCTTCCTGCGGCGTCGCAAGCGCTTCAA GCGGCAGCCCCTGCCGCCGCCGCACCCACACCCGCACCCTCACCCGGAGCTGCTGC TGCGTGGCGGGGCCGCGGCGGCGGGGGATCCCGGCGCTTTCCTGCCCGGCTTCGCT GCCTACGGCGCCTACGGCTACGGCTACGGGCTGGCTCTCCCGGCCTACGGCGCACC CCCGCCGGGGCCGGCCCCGCATCCGCACCCGCACCCGCACGCCTTCGCTTTCGCCG CGGCAGCCGCCGCCGCTCCTTGCCAGCTGTCGGTACCCCCAGGCCGCGCCGCCGCG CCTCCACCCGGACCTCCGACGGCCTCGGTGTTCGCAGGCGCGGGATCGGCCCCAGC TCCTGCGCCTGCCTCAGGCTCGGGCCCGGGCCCGGGCCCCGCAGGCCTGCCCGCCT TCCTGGGCGCGGAGCTGGGCTGCGCCAAAGCCTTCTACCCGGCGTCCCTGAGTCCTC CCGCAGCCGGCACCGCGGCGGGTCTGCCCACCGCACTTCTGCGCCAGGGCCTCAAG ACGGACGCGGGCGGTGGTGCAGGCGGCGGGGGCGCCGGGGCAGGGCAGAGGCCTT CCTTCTCTATAGACCACATCATGGGCCACGGTGGCGGCGGGGCAGCACCCCCGGGCG CCGGCGAGGGCTCTCCGGGACCGCCATTCGCGGCAGCCGCGGGTCCTGGGGGCCAA GCCCAGGTCTTGGCCATGCTGACTGCTCCGGCCCTGGCTCCCGTTGCTGGCCACATT CGCCTCTCGCATCCCGGGGACGCGCTGCTGTCCTCAGGGTCCCGGTTTGCCAGCAAA GTCGCCGGCCTTAGTGGCTGCCACTTC (SEQ ID NO:20; NM_004474), or a nucleic acid sequence that hybridizes to a nucleic acid sequence consisting of SEQ ID NO:20 under stringent hybridization conditions.

In some embodiments, Forkhead box D3 (FOXD3) comprises the amino acid sequence: MTLSGGGSASDMSGQTVLTAEDVDIDVVGEGDDGLEEKDSDAGCDSPAGPPELRLDEAD EVPPAAPHHGQPQPPHQQPLTLPKEAAGAGAGPGGDVGAPEADGCKGGVGGEEGGASG GGPGAGSGSAGGLAPSKPKNSLVKPPYSYIALITMAILQSPQKKLTLSGICEFISNRFPYYRE KFPAWQNSIRHNLSLNDCFVKIPREPGNPGKGNYWTLDPQSEDMFDNGSFLRRRKRFKR HQQEHLREQTALMMQSFGAYSLAAAAGAAGPYGRPYGLHPAAAAGAYSHPAAAAAAAAA AALQYPYALPPVAPVLPPAVPLLPSGELGRKAAAFGSQLGPGLQLQLNSLGAAAAAAGTAG AAGTTASLIKSEPSARPSFSIENIIGGGPAAPGGSAVGAGVAGGTGGSGGGSTAQSFLRPP GTVQSAALMATHQPLSLSRTTATIAPILSVPLSGQFLQPAASAAAAAAAAAQAKWPAQ (SEQ ID NO:21; NP_036315), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:21.

In some embodiments, the nucleic acid sequence encoding FOXD3 comprises the nucleic acid sequence: ATGACCCTCTCCGGCGGCGGCAGCGCCAGCGACATGTCCGGCCAGACGGTGCTGAC GGCCGAGGACGTGGACATCGATGTGGTGGGCGAGGGCGACGACGGGCTGGAAGAGA AGGACAGCGACGCAGGTTGCGATAGCCCCGCGGGGCCGCCGGAGCTGCGCCTGGAC GAGGCGGACGAGGTGCCCCCGGCGGCACCCCATCACGGACAGCCTCAGCCGCCCCA CCAGCAGCCCCTGACATTGCCCAAGGAGGCGGCCGGAGCCGGGGCCGGACCGGGG GGCGACGTGGGCGCGCCGGAGGCGGACGGCTGCAAGGGCGGTGTTGGCGGCGAGG AGGGCGGCGCGAGCGGCGGCGGGCCTGGCGCGGGCAGCGGTTCGGCGGGAGGCCT GGCCCCGAGCAAGCCCAAGAACAGCCTAGTGAAGCCGCCTTACTCGTACATCGCGCT CATCACCATGGCCATCCTGCAGAGCCCGCAGAAGAAGCTGACCCTGAGCGGCATCTG CGAGTTCATCAGCAACCGCTTCCCCTACTACAGGGAGAAGTTCCCCGCCTGGCAGAAC AGCATCCGCCACAACCTCTCACTCAACGACTGCTTCGTCAAGATCCCCCGCGAGCCGG GCAACCCGGGCAAGGGCAACTACTGGACCCTGGACCCGCAGTCCGAGGACATGTTCG ACAACGGCAGCTTCCTGCGGCGCCGGAAACGCTTCAAGCGCCACCAGCAGGAGCACC TGCGCGAGCAGACGGCGCTCATGATGCAGAGCTTCGGCGCTTACAGCCTGGCGGCGG CGGCCGGCGCCGCGGGACCCTACGGCCGCCCCTACGGCCTGCACCCTGCGGCGGC GGCCGGTGCCTATTCGCACCCGGCAGCGGCGGCGGCCGCGGCTGCTGCGGCGGCG CTCCAGTACCCGTACGCGCTGCCGCCGGTGGCACCGGTGCTGCCTCCCGCTGTGCCG CTGCTGCCCTCGGGCGAGCTGGGCCGCAAAGCGGCCGCCTTCGGCTCACAGCTCGG CCCGGGCCTGCAGCTGCAGCTCAATAGCCTGGGCGCCGCCGCGGCCGCTGCGGGCA CAGCGGGCGCCGCGGGCACCACCGCGTCGCTCATCAAGTCCGAGCCAAGCGCGCGG CCGTCGTTCAGCATCGAGAACATCATAGGTGGGGGCCCCGCGGCTCCTGGGGGCTCG GCGGTGGGCGCTGGGGTCGCCGGCGGCACTGGGGGTTCAGGGGGCGGCAGCACGG CGCAGTCGTTTCTGCGGCCACCCGGGACCGTGCAGTCGGCAGCGCTCATGGCCACCC ACCAACCGCTGTCGCTGAGCCGGACGACTGCCACCATCGCGCCCATTCTTAGCGTGC CACTCTCCGGACAGTTTCTGCAGCCCGCAGCCTCGGCCGCCGCCGCTGCTGCGGCCG CCGCTCAAGCCAAATGGCCGGCGCAA (SEQ ID NO:22; NM_012183), or a nucleic acid sequence that hybridizes to a nucleic acid sequence consisting of SEQ ID NO:22 under stringent hybridization conditions.

In some embodiments, Forkhead box D4 (FOXD4) comprises the amino acid sequence: MNLPRAERLRSTPQRSLRDSDGEDGKIDVLGEEEDEDEEEAASQQFLEQSLQPGLQVAR WGGVALPREHIEGGGGPSDPSEFGTEFRAPPRSAAASEDARQPAKPPSSYIALITMAILQS PHKRLTLSGICAFISDRFPYYRRKFPAWQNSIRHNLSLNDCFVKIPREPGRPGKGNYWSLD PASQDMFDNGSFLRRRKRFQRHQPTPGAHLPHPFPLPAAHAALHNPRPGPLLGAPAPPQ PVPGAYPNTGPGRRPYALLHPHPPRYLLLSAPAYAGAPKKAEGADLATPAPFPCCSPHLVL SLGRRARVWRRHREADASLSALRVSCKGSGERVQGLRRVCPRPRGATAPCSSDRQACR TILQQQQRHQEEDCANGCAPTKGAVLGGHLSAASALLRYQAVAEGSGLTSLAAPLGGEGT SPVFLVSPTPSSLAESAGPS (SEQ ID NO:23; NP_997188), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:23.

In some embodiments, the nucleic acid sequence encoding FOXD4 comprises the nucleic acid sequence: ATGAACTTGCCAAGAGCTGAGCGCCTTCGCTCCACACCGCAGCGCAGC CTCCGGGACTCCGATGGGGAAGACGGTAAAATCGATGTCCTGGGAGAGGAGGAAGAT GAAGACGAGGAGGAGGCGGCGAGCCAGCAGTTCCTAGAGCAGTCGCTCCAGCCGGG GCTGCAGGTGGCCCGGTGGGGCGGGGTTGCGCTTCCCCGAGAGCACATCGAGGGCG GCGGCGGCCCGAGCGACCCCTCAGAGTTTGGCACCGAGTTCAGGGCACCGCCAAGG TCTGCGGCGGCCTCTGAAGATGCCCGGCAGCCGGCAAAGCCCCCCTCCTCGTACATC GCGCTCATCACCATGGCCATCCTGCAAAGCCCGCACAAGCGCCTCACGCTCAGCGGC ATCTGCGCCTTCATTAGTGACCGCTTCCCCTACTACCGCCGCAAGTTCCCCGCCTGGC AGAACAGCATCCGCCACAACCTCTCGCTGAACGACTGCTTCGTCAAGATCCCCCGCGA GCCGGGCCGCCCAGGCAAGGGCAACTACTGGAGCCTGGACCCCGCCTCCCAGGACA TGTTCGACAATGGCAGCTTTCTCCGGCGTAGGAAGCGTTTCCAGCGCCACCAACCGAC CCCGGGAGCCCACCTGCCCCACCCCTTCCCTCTACCTGCTGCACACGCCGCCCTGCA CAACCCCCGCCCAGGCCCTCTGCTTGGGGCCCCTGCCCCGCCGCAGCCAGTCCCGG GGGCCTACCCCAACACCGGCCCCGGGAGACGCCCTTACGCTCTGCTGCACCCGCATC CTCCTCGCTACCTACTGCTCTCGGCCCCCGCCTATGCCGGGGCACCGAAGAAAGCAG AAGGCGCGGACCTGGCGACCCCGGCACCCTTCCCGTGCTGCAGCCCTCACTTGGTCC TCAGCCTTGGGAGGAGGGCAAGGGTCTGGCGTCGCCACCGGGAGGCGGATGCATCT CTTTCAGCATTGAGAGTATCATGCAAGGGGTCAGGGGAGCGGGTACAGGGGCTGCGC AGAGTTTGTCCCCGACCGCGTGGAGCTACTGCCCCCTGCTCCAGCGACCGTCAAGCC TGTCGGACAATTTTGCAGCAACAGCAGCGGCATCAGGAGGAGGACTGCGCCAACGGC TGCGCTCCCACCAAGGGCGCGGTGCTGGGCGGGCACCTGTCGGCCGCGTCGGCGCT GCTGCGGTATCAGGCGGTGGCAGAGGGCTCTGGGCTGACATCGCTGGCCGCCCCTTT GGGCGGAGAGGGGACCTCACCAGTTTTTTTAGTATCGCCCACGCCCAGTTCCCTGGCC GAGTCCGCAGGGCCCTCC (SEQ ID NO:24; NM_207305), or a nucleic acid sequence that hybridizes to a nucleic acid sequence consisting of SEQ ID NO:24 under stringent hybridization conditions.

In some embodiments, Forkhead box D4 like 1 (FOXD5) comprises the amino acid sequence: MNLPRAERPRSTPQRSLRDSDGEDGKIDVLGEEEDEDEVEDEEEEASQKFLEQSLQPGLQ VARWGGVALPREHIEGGGPSDPSEFGTEFRAPPRSAAASEDARQPAKPPYSYIALITMAIL QSPHKRLTLSGICAFISGRFPYYRRKFPAWQNSIRHNLSLNDCFVKIPREPGHPGKGTYWS LDPASQDMFDNGSFLRRRKRFKRHQLTPGAHLPHPFPLPAAHAALHNPRPGPLLGAPALP QPVPGAYPNTAPGRRPYALLHPHPPRYLLLSAPAYAGAPKKAEGADLATPGTLPVLQPSLG PQPWEEGKGLASPPGGGCISFSIESIMQGVRGAGTGAAQSLSPTAWSYCPLLQRPSSLSD NFAATAAASGGGLRQRLRSHQGRGAGRAPVGRVGAAAVSGGGRGL (SEQ ID NO:25; NP_036316), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:25.

In some embodiments, the nucleic acid sequence encoding FOXD5 comprises the nucleic acid sequence: ATCTTTGCCGGACGTTGTTGCAAAGGAGTAGAAACAAGCAGAGGAAAAC ATCCCAAAGGGTAACCACTAGCGTTCCTGCTTCTTGCAACATTCATCCCAGGCTTCCAG CTCAGCCCGCCCCGGGCCAGGTGATCGGCCGCCACATCCCCTGCGACTGAAGCACCT GCTCCGCCATGAACCTGCCAAGAGCTGAGCGCCCTCGCTCCACACCGCAGCGCAGCC TCCGGGACTCCGATGGGGAAGACGGTAAAATCGATGTCCTGGGAGAGGAGGAAGATG AAGACGAGGTGGAAGACGAGGAGGAGGAGGCGAGCCAGAAGTTCCTAGAGCAGTCG CTCCAGCCGGGGCTGCAGGTGGCCCGGTGGGGCGGGGTTGCGCTTCCCCGAGAGCA CATCGAGGGCGGCGGCCCGAGCGACCCCTCAGAGTTTGGCACCGAGTTCAGGGCAC CGCCAAGGTCTGCGGCGGCCTCTGAAGATGCCCGGCAGCCGGCAAAGCCCCCCTACT CGTACATCGCGCTCATCACCATGGCCATCCTGCAAAGCCCGCACAAGCGCCTCACGCT CAGCGGCATCTGCGCCTTCATTAGTGGCCGCTTCCCCTACTACCGCCGCAAGTTCCCC GCCTGGCAGAACAGCATCCGCCACAACCTCTCGCTGAACGACTGCTTCGTCAAGATCC CCCGCGAGCCGGGCCACCCAGGCAAGGGCACCTACTGGAGCCTGGACCCCGCCTCC CAGGACATGTTCGACAATGGCAGCTTTCTCCGGCGTAGGAAGCGTTTCAAGCGCCACC AACTGACCCCGGGAGCCCACCTGCCCCACCCCTTCCCTCTACCTGCTGCACACGCCG CCCTGCACAACCCCCGCCCAGGCCCTCTGCTTGGGGCCCCTGCCCTGCCGCAGCCAG TCCCGGGGGCCTACCCCAACACCGCCCCCGGGAGACGCCCTTACGCTCTGCTGCACC CGCATCCTCCTCGCTACCTACTGCTCTCGGCCCCCGCCTATGCCGGGGCACCGAAGA AAGCAGAAGGCGCGGACCTGGCGACCCCCGGCACCCTTCCCGTGCTGCAGCCCTCAC TTGGTCCTCAGCCTTGGGAGGAGGGCAAGGGTCTGGCGTCGCCACCGGGAGGCGGA TGCATCTCTTTCAGCATTGAGAGTATCATGCAAGGGGTCAGGGGAGCGGGTACAGGG GCTGCGCAGAGTTTGTCCCCGACCGCGTGGAGCTACTGCCCCCTGCTCCAGCGACCG TCAAGCCTGTCGGACAATTTTGCAGCAACAGCAGCAGCATCAGGAGGAGGACTGCGC CAACGGCTGCGCTCCCACCAAGGGCGCGGTGCTGGGCGGGCACCTGTCGGCCGCGT CGGCGCTGCTGCGGTATCAGGCGGTGGCAGAGGGCTCTAGGCTGACATCGCTGGCTG CCCCTTTGGGCGGAGAGGGGACCTCACCAGTTTTTTTAGTATCGCCCACGCCCAGTTC CCTGGCCAAGTCCGCAGGGCCCTCCTAGAGCCAGGTGGGAGTGGGGAGCGATCCGC AGCTGCTCACTCCACCTTGCGCGGCCCATACTGGGCGTGTGCATCTGAATCCTGCTGG AGAGCAAACACGAACTTCTGTTCCCTGCAAAATGGTTAGAAAGAAACAGCTGGATTACG TTCCTCTAAAAACCACCTGAACGTAACCTTCGCAGGGCGTCAAGTCATCTTTTCTTGCC TTCGGCTGTGGCTTCTGTGGCTTTCCGGATTTGCACATTTCCTGGGGTACTATGAACGT GAGTGGGGTATTTTGTTCTGGCATTAGAAGAAAAACAAGCAAGCAAACAAAAACACAGC CTCCGATGCCAAACATGTTCCCCCTTCTTCACTTCCTTGGAACTGGAAGTGTTATTCCT AAGTCTAGTGCAAAATGCTTCTACTCTCTGTGTCTTCCTGATAGGGATGTTTAATGTAAG TAGGATATTAATTTCAGAACATTGATTTCTTATCTGTGTGTCTGACGTGCCATCTTTAAT GTTAAAATTAAGGTGTTAAAATTAAGCCTAGTTATATAGACGAAATAAAATGCTAAGTCA CTA (SEQ ID NO:26; NM_012184.4), or a nucleic acid sequence that hybridizes to a nucleic acid sequence consisting of SEQ ID NO:26 under stringent hybridization conditions.

In some embodiments, Forkhead box D4 like 3 (FOXD6) comprises the amino acid sequence MNLPRAERLRSTPQRSLRDSDGEDGKIDVLGEEEDEDEVEDEEEAASQQFLEQSLQPGLQ VARWGGVALPREHIEGGGGPSDPSEFGTKFRAPPRSAAASEDARQPAKPPYSYIALITMAI LQNPHKRLTLSGICAFISGRFPYYRRKFPAWQNSIRHNLSLNDCFVKIPREPGHPGKGNYW SLDPASQDMFDNGSFLRRRKRFKRHQLTPGAHLPHPFPLPAAHAALHNPRPGPLLGAPAP PQPVPGAYPNTAPGRRPYALLHPHPLRYLLLSAPVYAGAPKKAEGAALATPAPFPCCSPHL VLSLGRRARVWRRHREADASLSALRVLCKGSRTAPTAALPPRARCWAGTCRPRRPC (SEQ ID NO:27; NP_954586), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:27.

In some embodiments, the nucleic acid sequence encoding FOXD6 comprises the nucleic acid sequence: CATTCATCCCAGGCTTCCAGCTCAGCCCGCCCCAGGCCAGGTGATCGGCCGCCACAT CCCCTGCGACTGAAGCACCTGCTCCTCCATGAACCTGCCAAGAGCTGAGCGCCTTCGC TCCACACCGCAGCGCAGCCTCCGGGACTCCGATGGGGAAGACGGTAAAATCGATGTC CTGGGAGAGGAGGAAGATGAAGACGAGGTGGAAGACGAGGAGGAGGCGGCGAGCCA GCAGTTCCTAGAGCAGTCGCTCCAGCCGGGGCTGCAGGTGGCCCGGTGGGGCGGGG TTGCGCTTCCCCGAGAGCACATCGAGGGCGGCGGCGGCCCGAGCGACCCCTCAGAG TTTGGCACCAAGTTCAGGGCACCGCCAAGGTCTGCGGCGGCCTCTGAAGATGCCCGG CAGCCGGCAAAGCCCCCCTACTCGTACATCGCGCTCATCACCATGGCCATCCTGCAAA ACCCGCACAAGCGCCTCACGCTCAGCGGCATCTGCGCCTTCATTAGTGGCCGCTTCCC CTACTACCGCCGCAAGTTCCCCGCCTGGCAGAACAGCATCCGCCACAACCTCTCGCTG AACGACTGCTTCGTTAAGATCCCCCGCGAGCCGGGCCACCCAGGCAAGGGCAACTAC TGGAGCCTGGACCCCGCCTCCCAAGACATGTTCGACAATGGCAGCTTTCTCCGGCGTA GGAAGCGTTTCAAGCGCCACCAACTGACCCCGGGAGCCCACCTGCCCCACCCCTTCC CTCTACCTGCTGCACACGCCGCCCTGCACAACCCCCGCCCAGGCCCTCTGCTTGGGG CCCCTGCCCCGCCGCAGCCAGTCCCGGGGGCCTACCCCAACACCGCCCCCGGGAGA CGCCCTTACGCTCTGCTGCACCCGCATCCTCTTCGCTACCTACTGCTCTCGGCCCCCG TCTATGCCGGGGCACCGAAGAAAGCAGAAGGCGCGGCCCTGGCGACCCCGGCACCC TTCCCGTGCTGCAGCCCTCACTTGGTCCTCAGCCTTGGGAGGAGGGCAAGGGTCTGG CGTCGCCACCGGGAGGCGGATGCATCTCTTTCAGCATTGAGAGTATTATGCAAGGGGT CAGGGGAGCGGGTACAGGGGCTGCGCAGAATTTGTCCCCGACCGCGTGGAGCTACTG CCACCTGCTCCAGCGACCATCAAGCCTGTTGCATCCCCAGACCGCTGCCCCTTTGCTG CAAGTGTCCGCCGCCGCCGCTGCTCGGACAATTTTGCAGCAATAGCAGCAGCATCAG GAGGAGGACTGCGCCAACGGCTGCGCTCCCACCAAGGGCGCGGTGCTGGGCGGGCA CCTGTCGGCCTCGTCGGCCCTGCTGAGGTATCAGGCAGTGGCAGAGGGCTCTAGGCT GACATCGCTGGCTGCCCCTTTGGGCGGAGAGGGGACCTCACCAGTTTTTTTAGTATCG CCCACGCCCAGTTCCCTGGCCAACTCCGCAGGGCCCTCCTAGAGCCAGGTGGGAGTG GGGAGCGACCCGCAGCTGCTCACTCCACCTTGCGCGGCCCATACTGGGCGTGTGCAT CTGAATCCCGCTGGAGAGCAAACACGAACTTCTGTTCGCTGCAAAATGGTTAGAAAGA AACAGCTGGATTACGTTCCTCTAAAAACCACCTGAACGTAACCTTCGCAGGGCGTCAA GTCATCTTTTCTTGCCTTCGGTTGTGGCTTCTATGGCTGTCCCGATTTGCGCATTTCCT GGGGTACTATGAACGTGAGTGGGGTATTTTGTTCTGGCATTAAAAGAAAAACAAGCAAG CAAACAAAAACACAGCCTCCGATGCCAAACATGTTCCCCCTTCTTCACTTCCTTGGAGC TGGAAGTATTATTCCTAAGTCTAGTGCAAAATGCTTCTACTCTCTGTGTCTTCCTGATAG GGATGTTTAATGTAAGTAGGATATTAATTTCAGAACATTGATTTCTTATCTGTGTGTCTG ACGTGCCATCTTTAATGTTAAAATTAAGGTGTTAAAATTAAGCCTAGTTATATAGACGAA ATAAAATGCTAAGTCACTACACTACATCGTTTATTTTCTATTACATCTCATTCTTCCCTTT CTAAATGGAACTTTTTAAAACCTACGTTATTTTCCCTCAAACAATTTATTTTCACAATTCA TATTTATTATAGATAGCAGAAGTAATCCATTTTAATATGGCCTTTAAAAATTCCAAATATT TGAGGTTGAAAATGTCCTGG (SEQ ID NO:28; NM_199135), or a nucleic acid sequence that hybridizes to a nucleic acid sequence consisting of SEQ ID NO:28 under stringent hybridization conditions.

In some embodiments, Forkhead box E1 (FOXE1/FOXE2) comprises the amino acid sequence: MTAESGPPPPQPEVLATVKEERGETAAGAGVPGEATGRGAGGRRRKRPLQRGKPPYSYI ALIAMAIAHAPERRLTLGGIYKFITERFPFYRDNPKKWQNSIRHNLTLNDCFLKIPREAGRPG KGNYWALDPNAEDMFESGSFLRRRKRFKRSDLSTYPAYMHDAAAAAAAAAAAAAAAAIFP GAVPAARPPYPGAVYAGYAPPSLAAPPPVYYPAASPGPCRVFGLVPERPLSPELGPAPSG PGGSCAFASAGAPATTTGYQPAGCTGARPANPSAYAAAYAGPDGAYPQGAGSAIFAAAG RLAGPASPPAGGSSGGVETTVDFYGRTSPGQFGALGACYNPGGQLGGASAGAYHARHA AAYPGGIDRFVSAM (SEQ ID NO:29; NP_004464), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:29.

In some embodiments, the nucleic acid sequence encoding FOXE1/FOXE2 comprises the nucleic acid sequence: ATGACTGCCGAGAGCGGGCCGCCGCCGCCGCAGCCGGAGGTGCTGGCTAC CGTGAAGGAAGAGCGCGGCGAGACGGCAGCAGGGGCCGGGGTCCCAGGGGAGGCC ACGGGCCGCGGGGCGGGCGGGCGGCGCCGCAAGCGCCCCCTGCAGCGCGGGAAGC CGCCCTACAGCTACATCGCGCTCATCGCCATGGCCATCGCGCACGCGCCCGAGCGCC GCCTCACGCTGGGCGGCATCTACAAGTTCATCACCGAGCGCTTCCCCTTCTACCGCGA CAACCCCAAAAAGTGGCAGAACAGCATCCGCCACAACCTCACACTCAACGACTGCTTC CTCAAGATCCCGCGCGAGGCCGGCCGCCCGGGTAAGGGCAACTACTGGGCGCTTGA CCCCAACGCGGAGGACATGTTCGAGAGCGGCAGCTTCCTGCGCCGCCGCAAGCGCTT CAAGCGCTCGGACCTCTCCACCTACCCGGCTTACATGCACGACGCGGCGGCTGCCGC AGCCGCCGCCGCCGCCGCCGCCGCCGCCGCCGCCATCTTCCCAGGCGCGGTGCCCG CCGCGCGCCCCCCCTACCCGGGCGCCGTCTATGCAGGCTACGCGCCGCCGTCGCTG GCCGCGCCGCCTCCAGTCTACTACCCCGCGGCGTCGCCCGGCCCTTGCCGCGTCTTC GGCCTGGTTCCTGAGCGGCCGCTCAGCCCAGAGCTGGGGCCCGCACCGTCGGGGCC CGGCGGCTCTTGCGCCTTTGCCTCCGCCGGGCCCCCGCTACCACCACCGGCTACCAG CCCGCAGGCTGCACCGGGGCCCGGCCGGCCAACCCCTCCGCCTATGCGGCTGCCTA CGCGGGCCCCGACGGCGCGTACCCGCAGGGCGCCGGCAGTGCGATCTTTGCCGCTG CTGGCCGCCTGGCGGGACCCGCTTCGCCCCCAGCGGGCGGCAGCAGTGGCGGCGTG GAGACCACGGTGGACTTCTACGGGCGCACGTCGCCCGGCCAGTTCGGAGCGCTGGG AGCCTGCTACAACCCTGGCGGGCAGCTCGGAGGGGCCAGTGCAGGCGCCTACCATG CTCGCCATGCTGCCGCTTATCCCGGTGGGATAGATCGGTTCGTGTCCGCCATG (SEQ ID NO:30; NM_004473), or a nucleic acid sequence that hybridizes to a nucleic acid sequence consisting of SEQ ID NO:30 under stringent hybridization conditions.

In some embodiments, Forkhead box E3 (FOXE3) comprises the amino acid sequence: MAGRSDMDPPAAFSGFPALPAVAPSGPPPSPLAGAEPGREPEEAAAGRGEAAPTPAPGP GRRRRRPLQRGKPPYSYIALIAMALAHAPGRRLTLAAIYRFITERFAFYRDSPRKWQNSIRH NLTLNDCFVKVPREPGNPGKGNYWTLDPAAADMFDNGSFLRRRKRFKRAELPAHAAAAP GPPLPFPYAPYAPAPGPALLVPPPSAGPGPSPPARLFSVDSLVNLQPELAGLGAPEPPCCA APDAAAAAFPPCAAAASPPLYSQVPDRLVLPATRPGPGPLPAEPLLALAGPAAALGPLSPG EAYLRQPGFASGLERYL (SEQ ID NO:31; NP_036318), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:31.

In some embodiments, the nucleic acid sequence encoding FOXE3 comprises the nucleic acid sequence: ATGGCGGGGCGCAGCGACATGGATCCGCCCGCCGCGTTCTCTGGCTTCCCTGCCCTG CCAGCGGTCGCGCCGTCGGGGCCGCCGCCGTCGCCGCTCGCAGGAGCCGAGCCAG GGCGGGAGCCAGAGGAGGCGGCGGCTGGCCGCGGAGAGGCGGCCCCCACGCCCGC GCCCGGCCCGGGGCGGCGGCGGCGGCGGCCCCTGCAGCGCGGGAAGCCGCCCTAC TCGTACATCGCGCTCATCGCCATGGCTCTGGCGCACGCCCCGGGCCGCCGCCTCACG CTGGCCGCCATCTACCGCTTCATCACCGAACGCTTTGCCTTCTACCGCGACAGCCCGC GCAAGTGGCAGAACAGCATCCGCCACAATCTCACGCTCAACGACTGCTTCGTCAAGGT GCCCCGCGAGCCGGGCAACCCGGGCAAGGGCAACTACTGGACGCTGGACCCCGCGG CCGCAGACATGTTCGACAACGGCAGCTTCCTGCGGCGCCGCAAGCGCTTCAAGCGCG CCGAGCTGCCCGCGCACGCGGCCGCGGCGCCAGGGCCGCCGCTCCCCTTCCCCTAC GCGCCCTACGCGCCCGCGCCCGGCCCCGCGCTGCTGGTGCCGCCGCCTTCTGCCGG ACCGGGCCCCTCGCCGCCCGCGCGTCTGTTCAGCGTCGACAGCCTGGTGAACCTGCA GCCGGAGCTAGCGGGGCTGGGCGCCCCCGAGCCGCCCTGCTGCGCCGCGCCCGAC GCCGCAGCCGCAGCCTTCCCGCCCTGCGCTGCCGCCGCCTCCCCGCCACTCTACTCG CAGGTCCCCGACCGCCTGGTACTGCCCGCGACGCGCCCCGGCCCCGGCCCGCTGCC CGCTGAGCCCCTCCTGGCCTTGGCCGGGCCGGCAGCCGCTCTCGGCCCGCTCAGCC CTGGGGAGGCCTACCTGAGGCAGCCGGGCTTCGCGTCGGGGCTGGAGCGCTACCTG (SEQ ID NO:32; NM_012186), or a nucleic acid sequence that hybridizes to a nucleic acid sequence consisting of SEQ ID NO:32 under stringent hybridization conditions.

In some embodiments, Forkhead box G1 (FOXG1) comprises the amino acid sequence: MLDMGDRKEVKMIPKSSFSINSLVPEAVQNDNHHASHGHHNSHHPQHHHHHHHHHHHPP PPAPQPPPPPQQQQPPPPPPPAPQPPQTRGAPAADDDKGPQQLLLPPPPPPPPAAALDG AKAVGLGGKGEPGGGPGELAPVGPDEKEKGAGAGGEEKKGAGEGGKDGEGGKEGEKK NGKYEKPPFSYNALIMMAIRQSPEKRLTLNGIYEFIMKNFPYYRENKQGWQNSIRHNLSLN MCFVKVPRHYDDPGKGNYWMLDPSSDDVFIGGTTGKLRRRSTTSRAKLAFKRGARLTSTG LTFMDRAGSLYWPMSPFLSLHHPRASSTLSYNGTTSAYPSHPMPYSSVLTQNSLGNNHSF STANGLSVDRLVNGEIPYATHHLTAAALAASVPCGLSVPCSGTYSLNPCSVNLLAGQTSYF FPHVPHPSMTSQSSTSMSARATSSSTSPQAPSTLPCESLRPSLPSFTTGLSGGLSDYFTHQ NQGSSSNPLIH (SEQ ID NO:33; NP_005240), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:33.

In some embodiments, the nucleic acid sequence encoding FOXG1 comprises the nucleic acid sequence: ATGCTGGACATGGGAGATAGGAAAGAGGTGAAAATGATCCCCAAGTCCTCGTTCAGCA TCAACAGCCTGGTGCCCGAGGCGGTCCAGAACGACAACCACCACGCGAGCCACGGCC ACCACAACAGCCACCACCCCCAGCACCACCACCACCACCACCACCATCACCACCACCC GCCGCCGCCCGCCCCGCAACCGCCGCCGCCGCCGCAGCAGCAGCAGCCGCCGCCG CCGCCGCCCCCGGCACCGCAGCCCCCCCAGACGCGGGGCGCCCCGGCCGCCGACG ACGACAAGGGCCCCCAGCAGCTGCTGCTCCCGCCGCCGCCACCGCCACCACCGGCC GCCGCCCTGGACGGGGCTAAAGCGGTCGGGCTGGGCGGCAAGGGCGAGCCGGGCG GCGGGCCGGGGGAGCTGGCGCCCGTCGGGCCGGACGAGAAGGAGAAGGGCGCCGG CGCCGGGGGGGAGGAGAAGAAGGGGGCGGGCGAGGGCGGCAAGGACGGGGAGGG GGGCAAGGAGGGCGAGAAGAAGAACGGCAAGTACGAGAAGCCGCCGTTCAGCTACAA CGCGCTCATCATGATGGCCATCCGGCAGAGCCCCGAGAAGCGGCTCACGCTCAACGG CATCTACGAGTTCATCATGAAGAACTTCCCTTACTACCGCGAGAACAAGCAGGGCTGG CAGAACTCCATCCGCCACAATCTGTCCCTCAACATGTGCTTCGTGAAGGTGCCGCGCC ACTACGACGACCCGGGCAAGGGCAACTACTGGATGCTGGACCCGTCGAGCGACGACG TGTTCATCGGCGGCACCACGGGCAAGCTGCGGCGCCGCTCCACCACCTCGCGGGCC AAGCTGGCCTTCAAGCGCGGTGCGCGCCTCACCTCCACCGGCCTCACCTTCATGGAC CGCGCCGGCTCCCTCTACTGGCCCATGTCGCCCTTCCTGTCCCTGCACCACCCCCGC GCCAGCAGCACTTTGAGTTACAACGGCACCACGTCGGCCTACCCCAGCCACCCCATG CCCTACAGCTCCGTGTTGACTCAGAACTCGCTGGGCAACAACCACTCCTTCTCCACCG CCAACGGCCTGAGCGTGGACCGGCTGGTCAACGGGGAGATCCCGTACGCCACGCAC CACCTCACGGCCGCCGCGCTAGCCGCCTCGGTGCCCTGCGGCCTGTCGGTGCCCTG CTCTGGGACCTACTCCCTCAACCCCTGCTCCGTCAACCTGCTCGCGGGCCAGACCAGT TACTTTTTCCCCCACGTCCCGCACCCGTCAATGACTTCGCAGAGCAGCACGTCCATGA GCGCCAGGGCCACGTCCTCCTCCACGTCGCCGCAGGCCCCCTCGACCCTGCCCTGTG AGTCTTTAAGACCCTCTTTGCCAAGTTTTACGACGGGACTGTCTGGGGGACTGTCTGAT TATTTCACACATCAAAATCAGGGGTCTTCTTCCAACCCTTTAATACAT (SEQ ID NO:34; NM_005249), or a nucleic acid sequence that hybridizes to a nucleic acid sequence consisting of SEQ ID NO:34 under stringent hybridization conditions.

In some embodiments, Forkhead box H1 (FOXH1) comprises the amino acid sequence: MGPCSGSRLGPPEAESPSQPPKRRKKRYLRHDKPPYTYLAMIALVIQAAPSRRLKLAQIIRQ VQAVFPFFREDYEGWKDSIRHNLSSNRCFRKVPKDPAKPQAKGNFWAVDVSLIPAEALRL QNTALCRRWQNGGARGAFAKDLGPYVLHGRPYRPPSPPPPPSEGFSIKSLLGGSGEGAP WPGLAPQSSPVPAGTGNSGEEAVPTPPLPSSERPLWPLCPLPGPTRVEGETVQGGAIGPS TLSPEPRAWPLHLLQGTAVPGGRSSGGHRASLWGQLPTSYLPIYTPNVVMPLAPPPTSCP QCPSTSPAYWGVAPETRGPPGLLCDLDALFQGVPPNKSIYDVWVSHPRDLAAPGPGWLL SWCSL (SEQ ID NO:35; NP_003914), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:35.

In some embodiments, the nucleic acid sequence encoding FOXH1 comprises the nucleic acid sequence: ATGGGGCCCTGCAGCGGCTCCCGCCTGGGGCCCCCAGAGGCAGAGTCGCC CTCCCAGCCCCCTAAGAGGAGGAAGAAGAGGTACCTGCGACATGACAAGCCCCCCTA CACCTACTTGGCCATGATCGCCTTGGTGATTCAGGCCGCTCCCTCCCGCAGACTGAAG CTGGCCCAGATCATCCGTCAGGTCCAGGCCGTGTTCCCCTTCTTCAGGGAAGACTACG AGGGCTGGAAAGACTCCATTCGCCACAACCTTTCCTCCAACCGATGCTTCCGCAAGGT GCCCAAGGACCCTGCAAAGCCCCAGGCCAAGGGCAACTTCTGGGCGGTCGACGTGAG CCTGATCCCAGCTGAGGCGCTCCGGCTGCAGAACACCGCCCTGTGCCGGCGCTGGCA GAACGGAGGTGCGCGTGGAGCCTTCGCCAAGGACCTGGGCCCCTACGTGCTGCACG GCCGGCCATACCGGCCGCCCAGTCCCCCGCCACCACCCAGTGAGGGCTTCAGCATCA AGTCCCTGCTAGGAGGGTCCGGGGAGGGGGCACCCTGGCCGGGGCTAGCTCCACAG AGCAGCCCAGTTCCTGCAGGCACAGGGAACAGTGGGGAGGAGGCGGTGCCCACCCC ACCCCTTCCCTCTTCTGAGAGGCCTCTGTGGCCCCTCTGCCCCCTTCCTGGCCCCACG AGAGTGGAGGGGGAGACTGTGCAGGGGGGAGCCATCGGGCCCTCAACCCTCTCCCC AGAGCCTAGGGCCTGGCCTCTCCACTTACTGCAGGGCACCGCAGTTCCTGGGGGACG GTCCAGCGGGGGACACAGGGCCTCCCTCTGGGGGCAGCTGCCCACCTCCTACTTGCC TATCTACACTCCCAATGTGGTAATGCCCTTGGCACCACCACCCACCTCCTGTCCCCAGT GTCCGTCAACCAGCCCTGCCTACTGGGGGGTGGCCCCTGAAACCCGAGGGCCCCCAG GGCTGCTCTGCGATCTAGACGCCCTCTTCCAAGGGGTGCCACCCAACAAAAGCATCTA CGACGTTTGGGTCAGCCACCCTCGGGACCTGGCGGCCCCTGGCCCAGGCTGGCTGCT CTCCTGGTGCAGCCTG (SEQ ID NO:36; NM_003923), or a nucleic acid sequence that hybridizes to a nucleic acid sequence consisting of SEQ ID NO:36 under stringent hybridization conditions.

In some embodiments, Forkhead box 11 (FOXI1) comprises the amino acid sequence: MSSFDLPAPSPPRCSPQFPSIGQEPPEMNLYYENFFHPQGVPSPQRPSFEGGGEYGATP NPYLWFNGPTMTPPPYLPGPNASPFLPQAYGVQRPLLPSVSGLGGSDLGWLPIPSQEELM KLVRPPYSYSALIAMAIHGAPDKRLTLSQIYQYVADNFPFYNKSKAGWQNSIRHNLSLNDCF KKVPRDEDDPGKGNYWTLDPNCEKMFDNGNFRRKRKRKSDVSSSTASLALEKTESSLPV DSPKTTEPQDILDGASPGGTTSSPEKRPSPPPSGAPCLNSFLSSMTAYVSGGSPTSHPLVT PGLSPEPSDKTGQNSLTFNSFSPLTNLSNHSGGGDWANPMPTNMLSYGGSVLSQFSPHF YNSVNTSGVLYPREGTEV (SEQ ID NO:37; NP_036320), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:37

In some embodiments, the nucleic acid sequence encoding FOXI1 comprises the nucleic acid sequence: ATGAGCTCCTTCGACCTGCCGGCGCCCTCCCCACCTCGCTGCAGCCCCCAGTTCCCC AGCATCGGCCAGGAGCCCCCCGAGATGAACCTCTACTATGAGAACTTCTTCCACCCAC AGGGCGTGCCCAGCCCTCAGCGGCCCTCCTTCGAGGGGGGCGGCGAGTATGGGGCC ACCCCCAACCCCTACCTCTGGTTCAACGGGCCCACCATGACCCCGCCACCCTACCTGC CCGGCCCCAACGCCAGCCCCTTCCTGCCCCAGGCCTATGGAGTGCAGAGACCGCTGC TGCCCAGCGTGTCGGGGCTTGGGGGGAGCGACCTGGGCTGGCTGCCCATCCCCTCG CAGGAGGAGCTGATGAAGCTGGTGCGGCCACCCTATTCCTACTCGGCTCTCATCGCCA TGGCCATCCACGGGGCACCCGACAAGCGCCTCACTCTCAGCCAGATCTACCAGTACGT GGCCGACAACTTCCCCTTCTACAACAAGAGCAAGGCCGGCTGGCAGAACTCCATCCGC CACAACCTGTCGCTCAACGACTGCTTCAAGAAGGTGCCCCGCGACGAGGACGACCCG GGCAAAGGGAATTACTGGACCCTGGACCCCAACTGTGAGAAAATGTTCGACAATGGAA ATTTCCGCAGGAAAAGGAAGAGAAAATCAGATGTTTCCTCTAGCACAGCCTCCTTGGCC TTAGAGAAGACAGAGAGCAGTCTCCCGGTGGACAGCCCCAAGACCACGGAGCCTCAG GACATCTTGGATGGAGCCTCACCAGGGGGCACCACCAGCTCCCCAGAGAAGCGGCCC TCCCCTCCCCCATCAGGCGCCCCTTGCCTTAACAGCTTCCTTTCCTCTATGACAGCCTA TGTGAGCGGGGGGAGCCCCACGAGCCACCCCTTGGTCACACCAGGACTGAGCCCTGA GCCCAGTGACAAGACGGGGCAGAACTCACTGACCTTCAACTCCTTCTCCCCGCTCACC AACCTCAGCAACCACAGCGGTGGGGGTGACTGGGCGAACCCCATGCCCACCAACATG CTCAGCTACGGAGGATCTGTGCTCAGCCAATTCAGCCCTCACTTCTACAACAGTGTCAA CACCAGTGGTGTCCTCTACCCCAGGGAGGGCACCGAGGTC (SEQ ID NO:38; NM_012188), or a nucleic acid sequence that hybridizes to a nucleic acid sequence consisting of SEQ ID NO:38 under stringent hybridization conditions.

In some embodiments, Forkhead box J1 (FOXJ1) comprises the amino acid sequence: MAESWLRLSGAGPAEEAGPEGGLEEPDALDDSLTSLQWLQEFSILNAKAPALPPGGTDPH GYHQVPGSAAPGSPLAADPACLGQPHTPGKPTSSCTSRSAPPGLQAPPPDDVDYATNPH VKPPYYATLICMAMQASKATKITLSAIYKWITDNFCYFRHADPTWQNSIRHNLSLNKCFIKVP REKDEPGKGGFWRIDPQYAERLLSGAFKKRRLPPVHIHPAFARQAAQEPSAVPRAGPLTV NTEAQQLLREFEEATGEAGWGAGEGRLGHKRKQPLPKRVAKVPRPPSTLLPTPEEQGELE PLKGNFDWEAIFDAGTLGGELGALEALELSPPLSPASHVDVDLTIHGRHIDCPATWGPSVE QAADSLDFDETFLATSFLQHPWDESGSGCLPPEPLFEAGDATLASDLQDWASVGAFL (SEQ ID NO:39; NP_001445), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:39.

In some embodiments, the nucleic acid sequence encoding FOXJ1 comprises the nucleic acid sequence: ATGGCGGAGAGCTGGCTGCGCCTCTCGGGAGCCGGGCCGGCGGAGGAGGCCGGGC CGGAGGGCGGCCTGGAGGAGCCCGACGCCCTGGATGACAGCCTGACCAGCCTGCAG TGGCTGCAGGAATTCTCCATTCTCAACGCCAAGGCCCCCGCCCTGCCCCCGGGGGGC ACCGACCCCCACGGCTACCACCAGGTGCCAGGTTCAGCGGCGCCCGGGTCCCCCCT GGCGGCCGACCCCGCCTGCCTGGGGCAGCCACACACGCCGGGCAAGCCCACGTCGT CGTGCACGTCGCGGAGCGCGCCCCCGGGGCTGCAGGCCCCACCCCCCGACGACGTG GACTACGCCACCAATCCGCACGTGAAGCCTCCCTACTCGTATGCCACGCTCATCTGCA TGGCCATGCAGGCCAGCAAGGCCACCAAGATCACCCTGTCGGCCATCTACAAGTGGAT CACGGACAACTTCTGCTACTTCCGCCACGCAGATCCCACCTGGCAGAATTCAATCCGC CACAACCTGTCTCTGAACAAGTGCTTCATCAAAGTGCCTCGGGAGAAGGACGAACCAG GCAAGGGGGGCTTCTGGCGCATTGACCCCCAGTACGCGGAGCGGCTACTGAGCGGC GCTTTCAAGAAGCGGCGACTGCCCCCTGTCCACATCCACCCAGCCTTTGCCCGCCAG GCCGCGCAGGAGCCCAGCGCTGTCCCCCGGGCCGGGCCGCTGACGGTGAATACCGA GGCCCAGCAGCTGCTGCGGGAGTTCGAGGAGGCCACCGGGGAGGCGGGCTGGGGT GCAGGCGAGGGCAGGCTGGGGCATAAGCGCAAACAGCCGCTGCCCAAGCGGGTGGC CAAGGTCCCGCGGCCCCCCAGCACCCTGCTGCCCACCCCGGAGGAGCAGGGTGAGC TGGAACCCCTCAAAGGCAACTTTGACTGGGAGGCCATCTTCGACGCCGGCACTCTGG GCGGGGAGCTGGGTGCACTGGAGGCCCTGGAGCTGAGCCCGCCTCTGAGCCCCGCC TCACACGTGGACGTGGACCTCACCATCCACGGCCGCCACATCGACTGCCCTGCCACC TGGGGGCCTTCGGTGGAGCAGGCTGCCGACAGCCTGGACTTCGATGAGACCTTCCTG GCCACATCCTTCCTGCAGCACCCCTGGGACGAGAGCGGCAGTGGCTGCCTGCCCCCG GAGCCCCTCTTTGAGGCTGGGGATGCCACCCTGGCCTCCGACCTGCAGGACTGGGCC AGCGTGGGGGCCTTCTTG (SEQ ID NO:40; NM_001454), or a nucleic acid sequence that hybridizes to a nucleic acid sequence consisting of SEQ ID NO:40 under stringent hybridization conditions.

In some embodiments, Forkhead box K1 (FOXK1) comprises the amino acid sequence: MAEVGEDSGARALLALRSAPCSPVLCAAAAAAAFPAAAPPPAPAQPQPPPGPPPPPPPPL PPGAIAGAGSSGGSSGVSGDSAVAGAAPALVAAAAASVRQSPGPALARLEGREFEFLMRQ PSVTIGRNSSQGSVDLSMGLSSFISRRHLQLSFQEPHFYLRCLGKNGVFVDGAFQRRGAP ALQLPKQCTFRFPSTAIKIQFTSLYHKEEAPASPLRPLYPQISPLKIHIPEPDLRSMVSPVPSP TGTISVPNSCPASPRGAGSSSYRFVQNVTSDLQLAAEFAAKAASEQQADTSGGDSPKDES KPPFSYAQLIVQAISSAQDRQLTLSGIYAHITKHYPYYRTADKGWQNSIRHNLSLNRYFIKVP RSQEEPGKGSFWRIDPASEAKLVEQAFRKRRQRGVSCFRTPFGPLSSRSAPASPTHPGLM SPRSGGLQTPECLSREGSPIPHDPEFGSKLASVPEYRYSQSAPGSPVSAQPVIMAVPPRPS SLVAKPVAYMPASIVTSQQPAGHAIHVVQQAPTVTMVRVVTTSANSANGYILTSQGAAGGS HDAAGAAVLDLGSEARGLEEKPTIAFATIPAAGGVIQTVASQMAPGVPGHTVTILQPATPVT LGQHHLPVRAVTQNGKHAVPTNSLAGNAYALTSPLQLLATQASSSAPVVVTRVCEVGPKE PAAAVAATATTTPATATTASASASSTGEPEVKRSRVEEPSGAVTTPAGVIAAAGPQGPGTG E (SEQ ID NO:41; NP_001032242), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:41.

In some embodiments, the nucleic acid sequence encoding FOXK1 comprises the nucleic acid sequence: ATGGCCGAAGTCGGCGAGGACAGCGGCGCCCGCGCCCTGCTCGCGCTGCGCTCGGC GCCCTGCAGCCCAGTGCTGTGCGCCGCAGCCGCCGCCGCCGCCTTCCCCGCGGCCG CACCCCCGCCGGCCCCCGCGCAGCCCCAGCCTCCGCCCGGGCCGCCGCCGCCGCC GCCACCGCCGCTGCCTCCGGGCGCGATCGCGGGCGCGGGCTCCTCCGGGGGCTCCT CCGGGGTATCCGGGGACTCCGCGGTCGCGGGCGCGGCGCCGGCCCTGGTGGCCGC GGCGGCCGCCTCGGTACGGCAGAGCCCGGGGCCGGCGCTGGCGCGGCTGGAGGGC CGCGAGTTCGAGTTCCTCATGCGCCAGCCCAGCGTCACCATCGGCCGCAACTCGTCG CAGGGCTCGGTGGACTTGAGCATGGGCCTGTCCAGCTTCATCTCGCGGCGCCACCTG CAGCTCAGCTTCCAGGAGCCGCACTTCTACCTGCGCTGCCTCGGCAAGAACGGCGTC TTCGTGGACGGGGCCTTCCAGAGACGCGGCGCGCCCGCCCTGCAGCTGCCCAAGCA GTGTACCTTCCGGTTTCCCAGCACGGCCATCAAGATCCAGTTCACGTCGCTCTATCACA AAGAAGAGGCCCCAGCCTCCCCGCTGCGGCCACTGTACCCCCAGATCTCCCCTCTGA AGATCCACATCCCGGAGCCGGACCTCCGGAGCATGGTCAGCCCCGTCCCCTCCCCGA CGGGCACCATCAGTGTCCCCAACTCCTGCCCAGCCAGTCCACGCGGTGCCGGCTCCT CCAGTTACCGCTTTGTGCAGAACGTGACCTCGGACCTGCAGCTGGCAGCAGAGTTTGC AGCAAAGGCCGCGTCGGAGCAGCAGGCAGACACGTCTGGAGGAGACAGCCCCAAGG ATGAGTCAAAGCCGCCGTTCTCCTACGCGCAGCTGATCGTGCAGGCCATCTCCTCCGC CCAGGACCGGCAGCTGACCCTGAGCGGGATCTACGCCCACATCACCAAGCATTACCC CTACTACCGGACGGCCGACAAAGGCTGGCAGAATTCTATCCGGCACAACCTCTCTTTG AACCGTTACTTTATCAAAGTCCCACGTTCCCAGGAGGAGCCTGGGAAGGGGTCCTTTT GGCGAATAGACCCTGCCTCTGAAGCCAAGCTCGTGGAACAGGCATTCCGGAAACGGA GGCAGAGGGGTGTCTCCTGCTTCCGCACCCCCTTCGGGCCTCTGTCCTCAAGGAGCG CTCCAGCTTCGCCCACACACCCCGGGCTGATGTCCCCTCGCTCCGGCGGCCTGCAGA CCCCAGAGTGCCTGTCTCGGGAGGGCTCCCCCATTCCACACGACCCTGAGTTTGGGT CCAAGTTAGCTTCTGTCCCAGAGTACCGGTATTCCCAAAGCGCACCCGGCTCCCCCGT CAGCGCCCAGCCAGTGATCATGGCCGTGCCTCCCCGACCGTCCAGCCTCGTGGCCAA GCCCGTGGCCTACATGCCCGCCTCCATCGTAACCTCACAGCAGCCCGCGGGCCACGC CATCCACGTCGTGCAGCAGGCCCCCACCGTCACCATGGTCAGGGTGGTCACCACATC TGCCAACTCGGCCAACGGATACATCCTCACCAGCCAGGGCGCGGCGGGGGGCTCCCA TGATGCGGCGGGCGCAGCCGTGCTGGACCTGGGCAGCGAGGCCAGAGGCCTGGAGG AGAAACCCACCATTGCGTTTGCCACAATCCCCGCGGCTGGTGGAGTCATCCAGACGGT GGCCAGCCAGATGGCCCCCGGGGTCCCCGGACACACGGTCACCATCCTGCAGCCCG CCACACCCGTGACCCTCGGGCAGCACCACCTTCCAGTCCGGGCCGTGACCCAGAACG GAAAGCATGCGGTTCCCACGAACAGTTTAGCCGGCAACGCTTACGCCCTCACCAGCCC TTTGCAGCTCCTTGCGACCCAAGCGAGTTCATCCGCGCCGGTGGTGGTCACCCGGGT GTGCGAGGTGGGGCCCAAGGAGCCAGCAGCAGCCGTCGCGGCCACGGCCACCACCA CCCCAGCCACTGCCACCACCGCCTCTGCCTCCGCCTCTTCCACTGGAGAGCCCGAGG TCAAAAGGTCCCGGGTGGAGGAGCCCAGTGGGGCTGTAACCACACCGGCTGGAGTGA TCGCAGCTGCCGGCCCCCAGGGGCCAGGCACCGGGGAG (SEQ ID NO:42; NM_001037165), or a nucleic acid sequence that hybridizes to a nucleic acid sequence consisting of SEQ ID NO:42 under stringent hybridization conditions.

In some embodiments, Forkhead box L1 (FOXL1) comprises the amino acid sequence: MSHLFDPRLPALAASPMLYLYGPERPGLPLAFAPAAALAASGRAETPQKPPYSYIALIAMAI QDAPEQRVTLNGIYQFIMDRFPFYHDNRQGWQNSIRHNLSLNDCFVKVPREKGRPGKGSY WTLDPRCLDMFENGNYRRRKRKPKPGPGAPEAKRPRAETHQRSAEAQPEAGSGAGGSG PAISRLQAAPAGPSPLLDGPSPPAPLHWPGTASPNEDAGDAAQGAAAVAVGQAARTGDG PGSPLRPASRSSPKSSDKSKSFSIDSILAGKQGQKPPSGDELLGGAKPGPGGRLGASLLAA SSSLRPPFNASLMLDPHVQGGFYQLGIPFLSYFPLQVPDTVLHFQ (SEQ ID NO:43; NP_005241), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:43.

In some embodiments, the nucleic acid sequence encoding FOXL1 comprises the nucleic acid sequence: ATGAGTCACCTCTTCGATCCCCGGCTGCCTGCCCTGGCCGCCTCGCCCATGCTGTATC TGTACGGTCCCGAGAGACCCGGCCTCCCTCTGGCCTTCGCCCCCGCGGCTGCTCTAG CTGCCTCGGGCCGGGCCGAGACCCCGCAGAAGCCTCCCTACAGCTACATCGCGCTCA TCGCCATGGCGATCCAGGACGCGCCCGAGCAGAGGGTCACGCTCAACGGCATCTACC AGTTCATCATGGACCGCTTCCCCTTCTACCACGACAACCGGCAGGGCTGGCAGAACAG CATCCGCCACAACCTCTCGCTCAACGACTGCTTCGTCAAGGTGCCCCGCGAGAAAGG GCGGCCGGGCAAGGGCAGCTACTGGACGCTGGACCCCCGCTGCCTGGACATGTTTGA GAACGGCAACTACCGGCGCCGGAAGAGGAAGCCCAAGCCGGGCCCCGGGGCCCCGG AGGCCAAGAGGCCCCGCGCCGAGACGCACCAGCGCAGCGCGGAGGCGCAGCCGGA GGCGGGGAGCGGGGCAGGGGGCTCGGGCCCCGCAATCTCCCGCCTGCAGGCAGCG CCCGCGGGCCCCTCGCCCCTCCTGGACGGCCCCTCTCCGCCGGCGCCCCTCCACTG GCCGGGGACCGCGTCCCCGAACGAGGACGCTGGTGACGCTGCCCAGGGCGCAGCGG CCGTGGCGGTCGGCCAGGCAGCGCGCACAGGGGACGGCCCGGGGTCCCCTCTGCG CCCCGCCTCCCGCAGCTCTCCGAAGAGCTCCGACAAGTCCAAGAGCTTCAGCATAGAC AGCATCCTGGCGGGAAAGCAGGGCCAGAAGCCGCCTTCAGGGGACGAACTCCTAGGG GGTGCCAAGCCTGGGCCCGGCGGCCGTCTGGGTGCCTCGCTCCTGGCCGCCTCCTC CAGCCTCCGTCCGCCTTTCAACGCTTCCCTGATGCTCGACCCGCATGTCCAGGGCGG CTTTTACCAGCTCGGGATCCCCTTCCTCTCTTATTTCCCCCTGCAGGTTCCCGACACGG TACTCCACTTCCAG (SEQ ID NO:44; NM_005250), or a nucleic acid sequence that hybridizes to a nucleic acid sequence consisting of SEQ ID NO:44 under stringent hybridization conditions.

In some embodiments, Forkhead box L2 (FOXL2) comprises the amino acid sequence MMASYPEPEDAAGALLAPETGRTVKEPEGPPPSPGKGGGGGGGTAPEKPDPAQKPPYSY VALIAMAIRESAEKRLTLSGIYQYIIAKFPFYEKNKKGWQNSIRHNLSLNECFIKVPREGGGE RKGNYWTLDPACEDMFEKGNYRRRRRMKRPFRPPPAHFQPGKGLFGAGGAAGGCGVA GAGADGYGYLAPPKYLQSGFLNNSWPLPQPPSPMPYASCQMAAAAAAAAAAAAAAGPGS PGAAAVVKGLAGPAASYGPYTRVQSMALPPGVVNSYNGLGGPPAAPPPPPHPHPHPHAH HLHAAAAPPPAPPHHGAAAPPPGQLSPASPATAAPPAPAPTSAPGLQFACARQPELAMMH CSYWDHDSKTGALHSRLDL (SEQ ID NO:45; NP_075555), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:45.

In some embodiments, the nucleic acid sequence encoding FOXL2 comprises the nucleic acid sequence: ATGATGGCCAGCTACCCCGAGCCCGAGGACGCGGCGGGGGCCCTGCTGGCCCCAGA GACCGGTCGCACAGTCAAGGAGCCAGAAGGGCCGCCGCCGAGCCCAGGCAAGGGCG GTGGGGGTGGCGGCGGGACAGCCCCGGAGAAGCCGGACCCGGCGCAGAAGCCCCC GTACTCGTACGTGGCGCTCATCGCCATGGCGATCCGCGAGAGCGCGGAGAAGAGGCT CACGCTGTCCGGCATCTACCAGTACATCATCGCGAAGTTCCCGTTCTACGAGAAGAAT AAGAAGGGCTGGCAAAATAGCATCCGCCACAACCTCAGCCTCAACGAGTGCTTCATCA AGGTGCCGCGCGAGGGCGGCGGCGAGCGCAAGGGCAACTACTGGACGCTGGACCCG GCCTGCGAAGACATGTTCGAGAAGGGCAACTACCGGCGCCGCCGCCGCATGAAGAGG CCCTTCCGGCCGCCGCCCGCGCACTTCCAGCCCGGCAAGGGGCTCTTCGGGGCCGG AGGCGCCGCAGGCGGGTGCGGCGTGGCGGGCGCCGGGGCCGACGGCTACGGCTAC CTGGCGCCCCCCAAGTACCTGCAGTCTGGCTTCCTCAACAACTCGTGGCCGCTACCGC AGCCTCCCTCACCCATGCCCTATGCCTCCTGCCAGATGGCGGCAGCCGCAGCGGCTG CAGCAGCTGCGGCTGCAGCCGCGGGCCCCGGTAGCCCTGGCGCGGCCGCTGTGGTC AAGGGGCTGGCGGGCCCGGCCGCCTCGTACGGGCCGTACACACGCGTGCAGAGCAT GGCGCTGCCCCCCGGCGTAGTGAACTCGTACAATGGCCTGGGAGGCCCGCCGGCCG CACCCCCGCCTCCGCCGCACCCCCACCCGCATCCGCACGCACACCATCTGCACGCGG CCGCCGCACCGCCGCCTGCCCCACCGCACCACGGGGCCGCCGCGCCGCCGCCGGG CCAGCTCAGCCCTGCCAGCCCAGCCACCGCCGCGCCCCCGGCGCCCGCGCCCACCA GTGCGCCGGGCCTGCAGTTCGCTTGTGCCCGGCAGCCCGAGCTCGCCATGATGCATT GCTCTTACTGGGACCACGACAGCAAGACCGGCGCGCTGCATTCGCGCCTCGATCTC (SEQ ID NO:46; NM_023067), or a nucleic acid sequence that hybridizes to a nucleic acid sequence consisting of SEQ ID NO:46 under stringent hybridization conditions.

In some embodiments, Forkhead box M1 (FOXM1) comprises the amino acid sequence: MKTSPRRPLILKRRRLPLPVQNAPSETSEEEPKRSPAQQESNQAEASKEVAESNSCKFPA GIKIINHPTMPNTQVVAIPNNANIHSIITALTAKGKESGSSGPNKFILISCGGAPTQPPGLRPQ TQTSYDAKRTEVTLETLGPKPAARDVNLPRPPGALCEQKRETCADGEAAGCTINNSLSNIQ WLRKMSSDGLGSRSIKQEMEEKENCHLEQRQVKVEEPSRPSASWQNSVSERPPYSYMA MIQFAINSTERKRMTLKDIYTWIEDHFPYFKHIAKPGWKNSIRHNLSLHDMFVRETSANGKV SFWTIHPSANRYLTLDQVFKPLDPGSPQLPEHLESQQKRPNPELRRNMTIKTELPLGARRK MKPLLPRVSSYLVPIQFPVNQSLVLQPSVKVPLPLAASLMSSELARHSKRVRIAPKVLLAEE GIAPLSSAGPGKEEKLLFGEGFSPLLPVQTIKEEEIQPGEEMPHLARPIKVESPPLEEWPSP APSFKEESSHSWEDSSQSPTPRPKKSYSGLRSPTRCVSEMLVIQHRERRERSRSRRKQHL LPPCVDEPELLFSEGPSTSRWAAELPFPADSSDPASQLSYSQEVGGPFKTPIKETLPISSTP SKSVLPRTPESWRLTPPAKVGGLDFSPVQTSQGASDPLPDPLGLMDLSTTPLQSAPPLESP QRLLSSEPLDLISVPFGNSSPSDIDVPKPGSPEPQVSGLAANRSLTEGLVLDTMNDSLSKILL DISFPGLDEDPLGPDNINWSQFIPELQ (SEQ ID NO:47; NP_068772), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:47.

In some embodiments, the nucleic acid sequence encoding FOXM1 comprises the nucleic acid sequence: ATGAAAACTAGCCCCCGTCGGCCACTGATTCTCAAAAGACGGAGGCTGCCCCTTCCTG TTCAAAATGCCCCAAGTGAAACATCAGAGGAGGAACCTAAGAGATCCCCTGCCCAACA GGAGTCTAATCAAGCAGAGGCCTCCAAGGAAGTGGCAGAGTCCAACTCTTGCAAGTTT CCAGCTGGGATCAAGATTATTAACCACCCCACCATGCCCAACACGCAAGTAGTGGCCA TCCCCAACAATGCTAATATTCACAGCATCATCACAGCACTGACTGCCAAGGGAAAAGAG AGTGGCAGTAGTGGGCCCAACAAATTCATCCTCATCAGCTGTGGGGGAGCCCCAACTC AGCCTCCAGGACTCCGGCCTCAAACCCAAACCAGCTATGATGCCAAAAGGACAGAAGT GACCCTGGAGACCTTGGGACCAAAACCTGCAGCTAGGGATGTGAATCTTCCTAGACCA CCTGGAGCCCTTTGCGAGCAGAAACGGGAGACCTGTGCAGATGGTGAGGCAGCAGGC TGCACTATCAACAATAGCCTATCCAACATCCAGTGGCTTCGAAAGATGAGTTCTGATGG ACTGGGCTCCCGCAGCATCAAGCAAGAGATGGAGGAAAAGGAGAATTGTCACCTGGA GCAGCGACAGGTTAAGGTTGAGGAGCCTTCGAGACCATCAGCGTCCTGGCAGAACTCT GTGTCTGAGCGGCCACCCTACTCTTACATGGCCATGATACAATTCGCCATCAACAGCA CTGAGAGGAAGCGCATGACTTTGAAAGACATCTATACGTGGATTGAGGACCACTTTCC CTACTTTAAGCACATTGCCAAGCCAGGCTGGAAGAACTCCATCCGCCACAACCTTTCCC TGCACGACATGTTTGTCCGGGAGACGTCTGCCAATGGCAAGGTCTCCTTCTGGACCAT TCACCCCAGTGCCAACCGCTACTTGACATTGGACCAGGTGTTTAAGCCACTGGACCCA GGGTCTCCACAATTGCCCGAGCACTTGGAATCACAGCAGAAACGACCGAATCCAGAGC TCCGCCGGAACATGACCATCAAAACCGAACTCCCCCTGGGCGCACGGCGGAAGATGA AGCCACTGCTACCACGGGTCAGCTCATACCTGGTACCTATCCAGTTCCCGGTGAACCA GTCACTGGTGTTGCAGCCCTCGGTGAAGGTGCCATTGCCCCTGGCGGCTTCCCTCATG AGCTCAGAGCTTGCCCGCCATAGCAAGCGAGTCCGCATTGCCCCCAAGGTGCTGCTA GCTGAGGAGGGGATAGCTCCTCTTTCTTCTGCAGGACCAGGGAAAGAGGAGAAACTCC TGTTTGGAGAAGGGTTTTCTCCTTTGCTTCCAGTTCAGACTATCAAGGAGGAAGAAATC CAGCCTGGGGAGGAAATGCCACACTTAGCGAGACCCATCAAAGTGGAGAGCCCTCCC TTGGAAGAGTGGCCCTCCCCGGCCCCATCTTTCAAAGAGGAATCATCTCACTCCTGGG AGGATTCGTCCCAATCTCCCACCCCAAGACCCAAGAAGTCCTACAGTGGGCTTAGGTC CCCAACCCGGTGTGTCTCGGAAATGCTTGTGATTCAACACAGGGAGAGGAGGGAGAG GAGCCGGTCTCGGAGGAAACAGCATCTACTGCCTCCCTGTGTGGATGAGCCGGAGCT GCTCTTCTCAGAGGGGCCCAGTACTTCCCGCTGGGCCGCAGAGCTCCCGTTCCCAGC AGACTCCTCTGACCCTGCCTCCCAGCTCAGCTACTCCCAGGAAGTGGGAGGACCTTTT AAGACACCCATTAAGGAAACGCTGCCCATCTCCTCCACCCCGAGCAAATCTGTCCTCC CCAGAACCCCTGAATCCTGGAGGCTCACGCCCCCAGCCAAAGTAGGGGGACTGGATT TCAGCCCAGTACAAACCTCCCAGGGTGCCTCTGACCCCTTGCCTGACCCCCTGGGGCT GATGGATCTCAGCACCACTCCCTTGCAAAGTGCTCCCCCCCTTGAATCACCGCAAAGG CTCCTCAGTTCAGAACCCTTAGACCTCATCTCCGTCCCCTTTGGCAACTCTTCTCCCTC AGATATAGACGTCCCCAAGCCAGGCTCCCCGGAGCCACAGGTTTCTGGCCTTGCAGC CAATCGTTCTCTGACAGAAGGCCTGGTCCTGGACACAATGAATGACAGCCTCAGCAAG ATCCTGCTGGACATCAGCTTTCCTGGCCTGGACGAGGACCCACTGGGCCCTGACAACA TCAACTGGTCCCAGTTTATTCCTGAGCTACAG (SEQ ID NO:48; NM_021953), or a nucleic acid sequence that hybridizes to a nucleic acid sequence consisting of SEQ ID NO:48 under stringent hybridization conditions.

In some embodiments, Forkhead box N1 (FOXN1) comprises the amino acid sequence: MVSLPPPQSDVTLPGPTRLEGERQGDLMQAPGLPGSPAPQSKHAGFSCSSFVSDGPPER TPSLPPHSPRIASPGPEQVQGHCPAGPGPGPFRLSPSDKYPGFGFEEAAASSPGRFLKGS HAPFHPYKRPFHEDVFPEAETTLALKGHSFKTPGPLEAFEEIPVDVAEAEAFLPGFSAEAW CNGLPYPSQEHGPQVLGSEVKVKPPVLESGAGMFCYQPPLQHMYCSSQPPFHQYSPGG GSYPIPYLGSSHYQYQRMAPQASTDGHQPLFPKPIYSYSILIFMALKNSKTGSLPVSEIYNF MTEHFPYFKTAPDGWKNSVRHNLSLNKCFEKVENKSGSSSRKGCLWALNPAKIDKMQEEL QKWKRKDPIAVRKSMAKPEELDSLIGDKREKLGSPLLGCPPPGLSGSGPIRPLAPPAGLSP PLHSLHPAPGPIPGKNPLQDLLMGHTPSCYGQTYLHLSPGLAPPGPPQPLFPQPDGHLELR AQPGTPQDSPLPAHTPPSHSAKLLAEPSPARTMHDTLLPDGDLGTDLDAINPSLTDFDFQG NLWEQLKDDSLALDPLVLVTSSPTSSSMPPPQPPPHCFPPGPCLTETGSGAGDLAAPGSG GSGALGDLHLTTLYSAFMELEPTPPTAPAGPSVYLSPSSKPVALA (SEQ ID NO:49; NP_003584), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:49.

In some embodiments, the nucleic acid sequence encoding FOXN1 comprises the nucleic acid sequence: ATGGTGTCGCTACCCCCGCCGCAGTCTGACGTCACGCTGCCGGGCCCCACCAGACTG GAGGGCGAGCGCCAAGGGGACCTCATGCAGGCACCGGGCCTCCCAGGCTCCCCTGC CCCACAGAGTAAGCATGCCGGCTTCAGCTGCTCGTCATTTGTGTCCGACGGCCCTCCA GAGAGGACACCCTCACTGCCCCCACACAGCCCCCGCATTGCGTCACCAGGGCCCGAG CAAGTCCAGGGCCACTGCCCAGCCGGCCCCGGCCCTGGGCCCTTCAGGCTCTCACCC TCAGACAAGTATCCTGGCTTTGGCTTTGAGGAGGCCGCAGCAAGCAGCCCTGGGCGA TTCCTCAAGGGCAGCCACGCGCCCTTCCACCCGTACAAGCGGCCTTTCCATGAGGAC GTCTTCCCAGAGGCCGAGACCACCCTGGCCCTCAAAGGACACTCCTTTAAGACCCCAG GGCCGCTGGAGGCCTTCGAGGAGATCCCAGTGGACGTGGCGGAGGCCGAGGCCTTC CTGCCTGGCTTCTCAGCAGAGGCCTGGTGTAACGGGCTCCCCTACCCCAGCCAGGAG CATGGCCCCCAAGTCCTGGGTTCAGAGGTCAAAGTCAAGCCCCCAGTTCTGGAGAGTG GTGCTGGGATGTTCTGCTACCAGCCTCCCTTGCAGCATATGTACTGCTCCTCCCAGCC CCCCTTCCACCAGTACTCGCCAGGTGGTGGCAGCTACCCCATACCCTACCTGGGCTCC TCACACTATCAGTACCAGCGAATGGCACCCCAGGCCAGCACCGATGGGCACCAGCCT CTCTTCCCAAAACCCATCTATTCCTACAGCATCCTCATCTTCATGGCCCTTAAGAACAGT AAAACTGGGAGCCTTCCCGTCAGCGAGATCTACAATTTTATGACGGAGCACTTTCCTTA CTTCAAGACAGCACCCGATGGCTGGAAGAATTCTGTCCGGCACAACCTATCCCTCAAC AAGTGCTTCGAGAAGGTGGAGAACAAATCAGGAAGTTCCTCCCGCAAGGGCTGCCTGT GGGCCCTCAATCCGGCCAAGATCGACAAGATGCAAGAGGAGCTGCAAAAATGGAAGA GGAAAGATCCCATTGCTGTGCGCAAAAGCATGGCCAAGCCAGAAGAGCTGGACAGCC TCATTGGAGACAAGAGAGAAAAGCTGGGCTCCCCACTCCTGGGCTGTCCGCCCCCTG GGCTGTCCGGCTCAGGCCCCATCCGGCCCCTGGCACCCCCAGCTGGCCTCTCCCCAC CACTGCACTCACTCCACCCAGCTCCAGGCCCCATTCCTGGCAAGAACCCCCTGCAGGA CCTACTTATGGGGCACACACCCTCCTGCTATGGGCAGACATACTTGCACCTCTCACCA GGCCTGGCCCCTCCTGGACCCCCGCAGCCATTGTTCCCACAGCCGGACGGGCACCTT GAGCTGCGGGCCCAGCCAGGCACCCCCCAGGACTCGCCTCTGCCTGCCCACACCCCA CCCAGCCACAGTGCCAAGCTACTGGCCGAGCCTTCCCCAGCCAGGACTATGCACGAC ACCCTGCTGCCAGATGGAGACCTTGGCACTGACCTGGATGCCATCAATCCCTCACTCA CTGACTTCGACTTCCAGGGAAACCTGTGGGAACAGTTGAAGGATGATAGCTTGGCCCT CGACCCCCTGGTACTGGTGACCTCATCCCCGACATCATCTTCGATGCCACCACCCCAG CCACCACCTCACTGCTTCCCCCCTGGGCCCTGTCTGACAGAGACAGGCAGTGGGGCA GGTGACTTGGCAGCCCCGGGCAGTGGTGGCTCCGGGGCACTGGGTGACCTGCACCT CACCACCCTCTACTCTGCCTTTATGGAGCTGGAGCCCACGCCCCCCACGGCCCCTGCA GGCCCCTCTGTGTACCTCAGCCCCAGCTCCAAGCCCGTGGCCCTGGCA (SEQ ID NO:50; NM_003593), or a nucleic acid sequence that hybridizes to a nucleic acid sequence consisting of SEQ ID NO:50 under stringent hybridization conditions.

In some embodiments, Forkhead box N2 (FOXN2) comprises the amino acid sequence: MGPVIGMTPDKRAETPGAEKIAGLSQIYKMGSLPEAVDAARPKATLVDSESADDELTNLNW LHESTNLLTNFSLGSEGLPIVSPLYDIEGDDVPSFGPACYQNPEKKSATSKPPYSFSLLIYMA IEHSPNKCLPVKEIYSWILDHFPYFATAPTGWKNSVRHNLSLNKCFQKVERSHGKVNGKGS LWCVDPEYKPNLIQALKKQPFSSASSQNGSLSPHYLSSVIKQNQVRNLKESDIDAAAAMML LNTSIEQGILECEKPLPLKTALQKKRSYGNAFHH PSAVRLQESDSLATSIDPKEDHNYSASS MAAQRCASRSSVSSLSSVDEVYEFIPKNSHVGSDGSEGFHSEEDTDVDYEDDPLGDSGYA SQPCAKISEKGQSGKKMRKQTCQEIDEELKEAAGSLLHLAGIRTCLGSLISTAKTQNQKQRK K (SEQ ID NO:51; NP_002149), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:51.

In some embodiments, the nucleic acid sequence encoding FOXN2 comprises the nucleic acid sequence: ATGGGTCCAGTAATTGGAATGACTCCAGATAAGAGAGCTGAAACCCCAGGAGCTGAAA AGATTGCAGGATTAAGCCAGATTTACAAAATGGGAAGCTTGCCTGAAGCTGTTGATGCT GCCAGGCCGAAGGCCACTCTAGTGGACAGTGAGTCAGCAGATGATGAACTCACAAACT TGAACTGGCTTCATGAAAGCACTAATCTTCTAACAAACTTCAGCCTCGGAAGTGAGGGT CTTCCAATTGTTAGTCCATTGTATGACATAGAGGGAGATGATGTGCCATCCTTTGGACC AGCTTGCTACCAGAACCCAGAAAAAAAATCAGCGACTTCAAAGCCCCCATACTCCTTTA GTCTTCTCATTTATATGGCCATTGAGCACTCTCCAAATAAATGTTTGCCTGTCAAAGAAA TTTATAGCTGGATTCTGGACCATTTTCCATATTTTGCTACTGCACCAACAGGCTGGAAG AATTCTGTTCGACATAATCTGTCCCTGAATAAATGTTTTCAGAAAGTGGAAAGAAGCCAT GGCAAGGTTAATGGAAAAGGTTCCTTATGGTGTGTTGATCCGGAATATAAACCCAATCT TATCCAGGCACTGAAGAAGCAACCTTTTTCTTCAGCATCTTCACAAAATGGTTCTTTATC ACCTCACTATTTAAGCTCTGTAATCAAGCAGAACCAGGTGCGAAACCTCAAAGAATCTG ATATTGATGCTGCTGCTGCAATGATGCTTTTAAATACTTCTATAGAACAAGGAATTTTAG AATGTGAGAAGCCTCTTCCTCTTAAAACAGCATTGCAAAAAAAGAGGAGTTACGGCAAT GCATTTCATCATCCCAGTGCTGTACGATTACAAGAGAGTGATTCTTTAGCCACCAGCAT TGATCCAAAAGAAGATCACAATTACAGTGCAAGTAGCATGGCAGCACAGCGTTGTGCAT CCAGGTCTAGCGTGTCTTCTCTGTCTTCTGTGGATGAGGTATATGAATTTATCCCAAAG AATAGTCACGTGGGAAGTGATGGCAGTGAAGGATTTCACAGTGAAGAAGATACAGACG TTGATTATGAAGATGATCCTCTTGGAGACAGTGGCTATGCATCACAGCCTTGTGCAAAA ATCTCTGAAAAAGGGCAGTCAGGCAAAAAGATGCGAAAACAGACATGTCAAGAAATTGA TGAGGAGCTCAAAGAGGCAGCTGGATCTCTGCTCCACCTTGCTGGAATTCGTACATGT TTAGGTTCCCTAATAAGTACTGCAAAGACACAAAATCAAAAGCAACGGAAAAAA (SEQ ID NO:52; NM_002158), or a nucleic acid sequence that hybridizes to a nucleic acid sequence consisting of SEQ ID NO:52 under stringent hybridization conditions.

In some embodiments, Forkhead box N3 (FOXN3) comprises the amino acid sequence: MGPVMPPSKKPESSGISVSSGLSQCYGGSGFSKALQEDDDLDFSLPDIRLEEGAMEDEEL TNLNWLHESKNLLKSFGESVLRSVSPVQDLDDDTPPSPAHSDMPYDARQNPNCKPPYSFS CLIFMAIEDSPTKRLPVKDIYNWILEHFPYFANAPTGWKNSVRHNLSLNKCFKKVDKERSQS IGKGSLWCIDPEYRQNLIQALKKTPYHPHPHVFNTPPTCPQAYQSTSGPPIWPGSTFFKRN GALLQDPDIDAASAMMLLNTPPEIQAGFPPGVIQNGARVLSRGLFPGVRPLPITPIGVTAAM RNGITSCRMRTESEPSCGSPVVSGDPKEDHNYSSAKSSNARSTSPTSDSISSSSSSADDH YEFATKGSQEGSEGSEGSFRSHESPSDTEEDDRKHSQKEPKDSLGDSGYASQHKKRQHF AKARKVPSDTLPLKKRRTEKPPESDDEEMKEAAGSLLHLAGIRSCLNNITNRTAKGQKEQK ETTKN (SEQ ID NO:53; NP_001078940), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:53.

In some embodiments, the nucleic acid sequence encoding FOXN3 comprises the nucleic acid sequence: ATGGGTCCAGTCATGCCTCCCAGTAAGAAGCCAGAAAGCTCAGGAATTAGTGTCTCCA GTGGACTGAGTCAGTGTTACGGGGGCAGCGGTTTCTCCAAGGCCCTTCAGGAAGACG ATGACCTCGACTTTTCTCTGCCTGACATCCGATTAGAAGAGGGGGCCATGGAAGATGA AGAGCTGACCAACCTGAACTGGCTGCACGAGAGCAAGAACTTGCTGAAGAGCTTTGGG GAGTCGGTCCTCAGGAGTGTCAGCCCCGTCCAGGACCTGGACGATGACACCCCCCCA TCCCCTGCCCACTCTGACATGCCCTACGATGCCAGGCAGAACCCCAACTGCAAACCCC CCTACTCCTTCAGCTGCCTCATATTTATGGCCATCGAGGACTCTCCAACCAAGCGCCTG CCAGTGAAGGATATCTACAACTGGATCTTGGAACATTTTCCGTATTTTGCAAATGCACCT ACTGGGTGGAAAAACTCAGTGAGACACAATTTATCATTGAATAAGTGTTTTAAGAAAGT GGACAAAGAGAGGAGTCAGAGTATTGGGAAAGGGTCGTTGTGGTGCATAGACCCAGA GTATAGACAAAATCTAATTCAGGCTTTGAAAAAGACACCTTATCACCCACACCCACACG TGTTCAATACACCTCCCACCTGTCCTCAGGCATATCAAAGCACATCAGGTCCACCCATC TGGCCGGGCAGTACCTTCTTCAAGAGAAATGGAGCCCTTCTCCAAGATCCTGACATTG ATGCTGCCAGTGCCATGATGCTTTTGAATACTCCCCCTGAGATACAAGCAGGTTTTCCT CCAGGAGTGATCCAAAATGGAGCGCGGGTCCTGAGCCGAGGGCTGTTTCCTGGCGTG CGGCCGCTGCCAATCACTCCCATTGGGGTGACAGCGGCCATGAGGAATGGCATCACC AGCTGCCGGATGCGGACTGAGAGTGAGCCATCTTGTGGCTCCCCAGTGGTCAGCGGA GACCCCAAGGAGGATCACAACTACAGCAGTGCCAAGTCCTCCAACGCCCGGAGCACC TCGCCCACCAGCGACTCCATCTCCTCCTCCTCCTCCTCAGCCGACGACCACTATGAGT TTGCCACCAAGGGGAGCCAGGAGGGCAGCGAGGGCAGCGAGGGGAGCTTCCGGAGC CACGAGAGCCCCAGCGACACGGAAGAGGACGACAGGAAGCACAGCCAGAAGGAGCC CAAGGATTCTCTGGGGGACAGCGGGTACGCATCCCAGCACAAGAAGCGCCAGCACTT CGCCAAGGCCAGGAAGGTCCCCAGCGACACACTGCCCCTCAAAAAGAGACGCACCGA AAAGCCCCCCGAGAGCGATGATGAGGAGATGAAAGAAGCGGCAGGGTCCCTCCTGCA CTTAGCAGGGATCCGGTCCTGTTTGAATAACATCACCAATCGGACGGCAAAGGGGCAG AAAGAGCAAAAGGAAACCACAAAAAAT (SEQ ID NO:54; NM_001085471), or a nucleic acid sequence that hybridizes to a nucleic acid sequence consisting of SEQ ID NO:54 under stringent hybridization conditions.

In some embodiments, Forkhead box N4 (FOXN4) comprises the amino acid sequence: MIESDTSSIMSGIIRNSGQNHHPSPQEYRLLATTSDDDLPGDLQSLSWLTAVDVPRLQQMA SGRVDLGGPCVPHPHPGALAGVADLHVGATPSPLLHGPAGMAPRGMPGLGPITGHRDSM SQFPVGGQPSSGLQDPPHLYSPATQPQFPLPPGAQQCPPVGLYGPPFGVRPPYPQPHVA VHSSQELHPKHYPKPIYSYSCLIAMALKNSKTGSLPVSEIYSFMKEHFPYFKTAPDGWKNSV RHNLSLNKCFEKVENKMSGSSRKGCLWALNLARIDKMEEEMHKWKRKDLAAIHRSMANPE ELDKLISDRPESCRRPGKPGEPEAPVLTHATTVAVAHGCLAVSQLPPQPLMTLSLQSVPLH HQVQPQAHLAPDSPAPAQTPPLHALPDLSPSPLPHPAMGRAPVDFINISTDMNTEVDALDP SIMDFALQGNLWEEMKDEGFSLDTLGAFADSPLGCDLGASGLTPASGGSDQSFPDLQVTG LYTAYSTPDSVAASGTSSSSQYLGAQGNKPIALL (SEQ ID NO:55; NP_998761), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:55.

In some embodiments, the nucleic acid sequence encoding FOXN4 comprises the nucleic acid sequence: ATGATAGAAAGTGACACCTCATCCATAATGTCAGGAATTATTCGAAACTCAGGGCAAAA TCACCACCCCTCTCCACAGGAATACAGGCTTCTAGCCACCACCAGCGATGATGACCTT CCCGGGGACCTGCAGTCGCTGTCGTGGCTCACGGCGGTGGATGTGCCTCGGCTGCA GCAGATGGCAAGTGGCCGCGTGGACCTGGGTGGCCCCTGCGTGCCACATCCACACCC AGGTGCCTTGGCTGGGGTGGCCGACCTGCATGTGGGAGCCACTCCAAGTCCCCTTCT CCATGGCCCAGCAGGCATGGCCCCCCGAGGCATGCCAGGTCTGGGCCCCATAACTGG CCACAGAGACAGCATGAGCCAGTTCCCCGTGGGGGGCCAGCCCTCATCTGGCCTGCA GGACCCGCCGCATCTGTACTCACCTGCCACCCAACCACAGTTCCCGCTCCCCCCGGG TGCCCAGCAGTGCCCTCCTGTGGGCCTCTATGGCCCCCCATTTGGGGTGCGGCCCCC CTACCCCCAGCCCCACGTGGCTGTGCATTCATCTCAAGAACTGCACCCCAAACACTAC CCCAAGCCCATCTACTCGTACAGCTGTCTGATCGCCATGGCCCTGAAGAACAGCAAGA CAGGCAGCCTGCCTGTGAGCGAGATCTACAGCTTCATGAAGGAGCACTTCCCCTACTT CAAGACGGCCCCCGACGGGTGGAAGAACTCGGTGCGGCACAACCTGTCTCTGAACAA GTGCTTCGAGAAGGTGGAGAACAAGATGAGCGGCTCCTCCCGCAAGGGCTGCCTGTG GGCTCTGAACCTGGCCCGCATCGACAAGATGGAGGAGGAGATGCACAAGTGGAAGAG GAAGGACCTGGCTGCCATCCACCGGAGTATGGCCAACCCTGAGGAGTTGGACAAGCT GATCTCCGACCGGCCTGAAAGCTGCCGGCGCCCCGGCAAACCGGGGGAACCAGAGG CCCCCGTGCTGACTCACGCCACCACAGTGGCCGTGGCGCATGGCTGCCTGGCTGTCT CCCAGCTCCCACCCCAGCCACTGATGACCCTGTCCCTGCAGTCAGTCCCCCTGCACCA CCAGGTCCAGCCCCAGGCACATCTTGCTCCAGACTCTCCAGCACCAGCCCAGACCCC GCCACTGCACGCCCTGCCGGACCTCAGCCCCAGCCCGCTCCCCCACCCCGCCATGG GAAGGGCTCCTGTAGACTTCATCAACATCAGCACCGACATGAACACTGAGGTGGATGC CCTCGACCCGAGCATCATGGACTTCGCTCTGCAGGGGAACCTGTGGGAGGAGATGAA GGATGAGGGATTCAGCTTGGACACACTGGGCGCCTTTGCAGACTCCCCGCTTGGCTGT GACCTGGGGGCCTCAGGCCTAACCCCTGCCTCGGGTGGCAGCGACCAGTCCTTCCCA GACTTGCAGGTGACGGGTCTCTACACAGCGTACTCCACTCCGGACAGTGTGGCTGCAT CGGGCACCAGCTCCTCCTCCCAGTACCTGGGTGCACAGGGGAACAAGCCTATAGCCC TGCTT (SEQ ID NO:56; NM_213596), or a nucleic acid sequence that hybridizes to a nucleic acid sequence consisting of SEQ ID NO:56 under stringent hybridization conditions.

In some embodiments, Forkhead Box 01 (FOXO1) comprises the amino acid sequence: MAEAPQVVEIDPDFEPLPRPRSCTWPLPRPEFSQSNSATSSPAPSGSAAANPDAAAGLPS ASAAAVSADFMSNLSLLEESEDFPQAPGSVAAAVAAAAAAAATGGLCGDFQGPEAGCLHP APPQPPPPGPLSQHPPVPPAAAGPLAGQPRKSSSSRRNAWGNLSYADLITKAIESSAEKRL TLSQIYEWMVKSVPYFKDKGDSNSSAGWKNSIRHNLSLHSKFIRVQNEGTGKSSWWMLNP EGGKSGKSPRRRAASMDNNSKFAKSRSRAAKKKASLQSGQEGAGDSPGSQFSKWPASP GSHSNDDFDNWSTFRPRTSSNASTISGRLSPIMTEQDDLGEGDVHSMVYPPSAAKMASTL PSLSEISNPENMENLLDNLNLLSSPTSLTVSTQSSPGTMMQQTPCYSFAPPNTSLNSPSPN YQKYTYGQSSMSPLPQMPIQTLQDNKSSYGGMSQYNCAPGLLKELLTSDSPPHNDIMTPV DPGVAQPNSRVLGQNVMMGPNSVMSTYGSQVSHNKMMNPSSHTHPGHAQQTSAVNGR PLPHTVSTMPHTSGMNRLTQVKTPVQVPLPHPMQMSALGGYSSVSSCNGYGRMGLLHQE KLPSDLDGMFIERLDCDMESIIRNDLMDGDTLDFNFDNVLPNQSFPHSVKTTTHSWVSG (SEQ ID NO:57; NP_002006), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:57.

In some embodiments, the nucleic acid sequence encoding FOXO1 comprises the nucleic acid sequence: ATGGCCGAGGCGCCTCAGGTGGTGGAGATCGACCCGGACTTCGAGCCGCTGCCCCG GCCGCGCTCGTGCACCTGGCCGCTGCCCAGGCCGGAGTTTAGCCAGTCCAACTCGGC CACCTCCAGCCCGGCGCCGTCGGGCAGCGCGGCTGCCAACCCCGACGCCGCGGCGG GCCTGCCCTCGGCCTCGGCTGCCGCTGTCAGCGCCGACTTCATGAGCAACCTGAGCT TGCTGGAGGAGAGCGAGGACTTCCCGCAGGCGCCCGGCTCCGTGGCGGCGGCGGTG GCGGCGGCGGCCGCCGCGGCCGCCACCGGGGGGCTGTGCGGGGACTTCCAGGGCC CGGAGGCGGGCTGCCTGCACCCAGCGCCACCGCAGCCCCCGCCGCCCGGGCCGCT GTCGCAGCACCCGCCGGTGCCCCCCGCCGCCGCTGGGCCGCTCGCGGGGCAGCCG CGCAAGAGCAGCTCGTCCCGCCGCAACGCGTGGGGCAACCTGTCCTACGCCGACCTC ATCACCAAGGCCATCGAGAGCTCGGCGGAGAAGCGGCTCACGCTGTCGCAGATCTAC GAGTGGATGGTCAAGAGCGTGCCCTACTTCAAGGATAAGGGTGACAGCAACAGCTCG GCGGGCTGGAAGAATTCAATTCGTCATAATCTGTCCCTACACAGCAAGTTCATTCGTGT GCAGAATGAAGGAACTGGAAAAAGTTCTTGGTGGATGCTCAATCCAGAGGGTGGCAAG AGCGGGAAATCTCCTAGGAGAAGAGCTGCATCCATGGACAACAACAGTAAATTTGCTA AGAGCCGAAGCCGAGCTGCCAAGAAGAAAGCATCTCTCCAGTCTGGCCAGGAGGGTG CTGGGGACAGCCCTGGATCACAGTTTTCCAAATGGCCTGCAAGCCCTGGCTCTCACAG CAATGATGACTTTGATAACTGGAGTACATTTCGCCCTCGAACTAGCTCAAATGCTAGTA CTATTAGTGGGAGACTCTCACCCATTATGACCGAACAGGATGATCTTGGAGAAGGGGA TGTGCATTCTATGGTGTACCCGCCATCTGCCGCAAAGATGGCCTCTACTTTACCCAGTC TGTCTGAGATAAGCAATCCCGAAAACATGGAAAATCTTTTGGATAATCTCAACCTTCTCT CATCACCAACATCATTAACTGTTTCGACCCAGTCCTCACCTGGCACCATGATGCAGCAG ACGCCGTGCTACTCGTTTGCGCCACCAAACACCAGTTTGAATTCACCCAGCCCAAACTA CCAAAAATATACATATGGCCAATCCAGCATGAGCCCTTTGCCCCAGATGCCTATACAAA CACTTCAGGACAATAAGTCGAGTTATGGAGGTATGAGTCAGTATAACTGTGCGCCTGG ACTCTTGAAGGAGTTGCTGACTTCTGACTCTCCTCCCCATAATGACATTATGACACCAG TTGATCCTGGGGTAGCCCAGCCCAACAGCCGGGTTCTGGGCCAGAACGTCATGATGG GCCCTAATTCGGTCATGTCAACCTATGGCAGCCAGGTATCTCATAACAAAATGATGAAT CCCAGCTCCCATACCCACCCTGGACATGCTCAGCAGACATCTGCAGTTAACGGGCGTC CCCTGCCCCACACGGTAAGCACCATGCCCCACACCTCGGGTATGAACCGCCTGACCC AAGTGAAGACACCTGTACAAGTGCCTCTGCCCCACCCCATGCAGATGAGTGCCCTGGG GGGCTACTCCTCCGTGAGCAGCTGCAATGGCTATGGCAGAATGGGCCTTCTCCACCAG GAGAAGCTCCCAAGTGACTTGGATGGCATGTTCATTGAGCGCTTAGACTGTGACATGG AATCCATCATTCGGAATGACCTCATGGATGGAGATACATTGGATTTTAACTTTGACAATG TGTTGCCCAACCAAAGCTTCCCACACAGTGTCAAGACAACGACACATAGCTGGGTGTC AGGC (SEQ ID NO:58; NM_002015), or a nucleic acid sequence that hybridizes to a nucleic acid sequence consisting of SEQ ID NO:58 under stringent hybridization conditions.

In some embodiments, Forkhead Box 03 (FOXO3) comprises the amino acid sequence: MAEAPASPAPLSPLEVELDPEFEPQSRPRSCTWPLQRPELQASPAKPSGETAADSMIPEE EDDEDDEDGGGRAGSAMAIGGGGGSGTLGSGLLLEDSARVLAPGGQDPGSGPATAAGG LSGGTQALLQPQQPLPPPQPGAAGGSGQPRKCSSRRNAWGNLSYADLITRAIESSPDKRL TLSQIYEWMVRCVPYFKDKGDSNSSAGWKNSIRHNLSLHSRFMRVQNEGTGKSSWWIINP DGGKSGKAPRRRAVSMDNSNKYTKSRGRAAKKKAALQTAPESADDSPSQLSKWPGSPTS RSSDELDAWTDFRSRTNSNASTVSGRLSPIMASTELDEVQDDDAPLSPMLYSSSASLSPSV SKPCTVELPRLTDMAGTMNLNDGLTENLMDDLLDNITLPPSQPSPTGGLMQRSSSFPYTTK GSGLGSPTSSFNSTVFGPSSLNSLRQSPMQTIQENKPATFSSMSHYGNQTLQDLLTSDSLS HSDVMMTQSDPLMSQASTAVSAQNSRRNVMLRNDPMMSFAAQPNQGSLVNQNLLHHQH QTQGALGGSRALSNSVSNMGLSESSSLGSAKHQQQSPVSQSMQTLSDSLSGSSLYSTSA NLPVMGHEKFPSDLDLDMFNGSLECDMESIIRSELMDADGLDFNFDSLISTQNVVGLNVGN FTGAKQASSQSWVPG (SEQ ID NO:59; NP_001446), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:59.

In some embodiments, the nucleic acid sequence encoding FOXO3 comprises the nucleic acid sequence: ATGGCAGAGGCACCGGCTTCCCCGGCCCCGCTCTCTCCGCTCGAAGTGGAGCTGGAC CCGGAGTTCGAGCCCCAGAGCCGTCCGCGATCCTGTACGTGGCCCCTGCAAAGGCCG GAGCTCCAAGCGAGCCCTGCCAAGCCCTCGGGGGAGACGGCCGCTGACTCCATGATC CCCGAGGAGGAGGACGATGAAGACGACGAGGACGGCGGGGGACGGGCCGGCTCGG CCATGGCGATCGGCGGCGGCGGCGGGAGCGGCACGCTGGGCTCCGGGCTGCTCCTT GAGGACTCGGCCCGGGTGCTGGCACCCGGAGGGCAAGACCCCGGGTCTGGGCCAGC CACCGCGGCGGGCGGGCTGAGCGGGGGTACACAGGCGCTGCTGCAGCCTCAGCAAC CGCTGCCACCGCCGCAGCCGGGGGCGGCTGGGGGCTCCGGGCAGCCGAGGAAATGT TCGTCGCGGCGGAACGCCTGGGGAAACCTGTCCTACGCGGACCTGATCACCCGCGCC ATCGAGAGCTCCCCGGACAAACGGCTCACTCTGTCCCAGATCTACGAGTGGATGGTGC GTTGCGTGCCCTACTTCAAGGATAAGGGCGACAGCAACAGCTCTGCCGGCTGGAAGA ACTCCATCCGGCACAACCTGTCACTGCATAGTCGATTCATGCGGGTCCAGAATGAGGG AACTGGCAAGAGCTCTTGGTGGATCATCAACCCTGATGGGGGGAAGAGCGGAAAAGC CCCCCGGCGGCGGGCTGTCTCCATGGACAATAGCAACAAGTATACCAAGAGCCGTGG CCGCGCAGCCAAGAAGAAGGCAGCCCTGCAGACAGCCCCCGAATCAGCTGACGACAG TCCCTCCCAGCTCTCCAAGTGGCCTGGCAGCCCCACGTCACGCAGCAGTGATGAGCT GGATGCGTGGACGGACTTCCGTTCACGCACCAATTCTAACGCCAGCACAGTCAGTGGC CGCCTGTCGCCCATCATGGCAAGCACAGAGTTGGATGAAGTCCAGGACGATGATGCG CCTCTCTCGCCCATGCTCTACAGCAGCTCAGCCAGCCTGTCACCTTCAGTAAGCAAGC CGTGCACGGTGGAACTGCCACGGCTGACTGATATGGCAGGCACCATGAATCTGAATGA TGGGCTGACTGAAAACCTCATGGACGACCTGCTGGATAACATCACGCTCCCGCCATCC CAGCCATCGCCCACTGGGGGACTCATGCAGCGGAGCTCTAGCTTCCCGTATACCACCA AGGGCTCGGGCCTGGGCTCCCCAACCAGCTCCTTTAACAGCACGGTGTTCGGACCTT CATCTCTGAACTCCCTACGCCAGTCTCCCATGCAGACCATCCAAGAGAACAAGCCAGC TACCTTCTCTTCCATGTCACACTATGGTAACCAGACACTCCAGGACCTGCTCACTTCGG ACTCACTTAGCCACAGCGATGTCATGATGACACAGTCGGACCCCTTGATGTCTCAGGC CAGCACCGCTGTGTCTGCCCAGAATTCCCGCCGGAACGTGATGCTTCGCAATGATCCG ATGATGTCCTTTGCTGCCCAGCCTAACCAGGGAAGTTTGGTCAATCAGAACTTGCTCCA CCACCAGCACCAAACCCAGGGCGCTCTTGGTGGCAGCCGTGCCTTGTCGAATTCTGTC AGCAACATGGGCTTGAGTGAGTCCAGCAGCCTTGGGTCAGCCAAACACCAGCAGCAG TCTCCTGTCAGCCAGTCTATGCAAACCCTCTCGGACTCTCTCTCAGGCTCCTCCTTGTA CTCAACTAGTGCAAACCTGCCCGTCATGGGCCATGAGAAGTTCCCCAGCGACTTGGAC CTGGACATGTTCAATGGGAGCTTGGAATGTGACATGGAGTCCATTATCCGTAGTGAACT CATGGATGCTGATGGGTTGGATTTTAACTTTGATTCCCTCATCTCCACACAGAATGTTGT TGGTTTGAACGTGGGGAACTTCACTGGTGCTAAGCAGGCCTCATCTCAGAGCTGGGTG CCAGGC (SEQ ID NO:60; NM_001455), or a nucleic acid sequence that hybridizes to a nucleic acid sequence consisting of SEQ ID NO:60 under stringent hybridization conditions.

In some embodiments, Forkhead Box 04 (FOXO4) comprises the amino acid sequence: MDPGNENSATEAAAIIDLDPDFEPQSRPRSCTWPLPRPEIANQPSEPPEVEPDLGEKVHTE GRSEPILLPSRLPEPAGGPQPGILGAVTGPRKGGSRRNAWGNQSYAELISQAIESAPEKRL TLAQIYEWMVRTVPYFKDKGDSNSSAGWKNSIRHNLSLHSKFIKVHNEATGKSSWWMLNP EGGKSGKAPRRRAASMDSSSKLLRGRSKAPKKKPSVLPAPPEGATPTSPVGH FAKWSGS PCSRNREEADMWTTFRPRSSSNASSVSTRLSPLRPESEVLAEEIPASVSSYAGGVPPTLNE GLELLDGLNLTSSHSLLSRSGLSGFSLQHPGVTGPLHTYSSSLFSPAEGPLSAGEGCFSSS QALEALLTSDTPPPPADVLMTQVDPILSQAPTLLLLGGLPSSSKLATGVGLCPKPLEAPGPS SLVPTLSMIAPPPVMASAPIPKALGTPVLTPPTEAASQDRMPQDLDLDMYMENLECDMDNII SDLMDEGEGLDFNFEPDP (SEQ ID NO:61; NP_005929), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:61.

In some embodiments, the nucleic acid sequence encoding FOXO4 comprises the nucleic acid sequence: ATGGATCCGGGGAATGAGAATTCAGCCACAGAGGCTGCCGCGATCATAGACCTAGATC CCGACTTCGAACCCCAGAGCCGTCCCCGCTCCTGCACCTGGCCCCTTCCCCGACCAG AGATCGCTAACCAGCCGTCCGAGCCGCCCGAGGTGGAGCCAGATCTGGGGGAAAAGG TACACACGGAGGGGCGCTCAGAGCCGATCCTGTTGCCCTCTCGGCTCCCAGAGCCGG CCGGGGGCCCCCAGCCCGGAATCCTGGGGGCTGTAACAGGTCCTCGGAAGGGAGGC TCCCGCCGGAATGCCTGGGGAAATCAGTCATATGCAGAACTCATCAGCCAGGCCATTG AAAGCGCCCCGGAGAAGCGACTGACACTTGCCCAGATCTACGAGTGGATGGTCCGTA CTGTACCCTACTTCAAGGACAAGGGTGACAGCAACAGCTCAGCAGGATGGAAGAACTC GATCCGCCACAACCTGTCCCTGCACAGCAAGTTCATCAAGGTTCACAACGAGGCCACC GGCAAAAGCTCTTGGTGGATGCTGAACCCTGAGGGAGGCAAGAGCGGCAAAGCCCCC CGCCGCCGGGCCGCCTCCATGGATAGCAGCAGCAAGCTGCTCCGGGGCCGCAGTAA AGCCCCCAAGAAGAAACCATCTGTGCTGCCAGCTCCACCCGAAGGTGCCACTCCAACG AGCCCTGTCGGCCACTTTGCCAAGTGGTCAGGCAGCCCTTGCTCTCGAAACCGTGAAG AAGCCGATATGTGGACCACCTTCCGTCCACGAAGCAGTTCAAATGCCAGCAGTGTCAG CACCCGGCTGTCCCCCTTGAGGCCAGAGTCTGAGGTGCTGGCGGAGGAAATACCAGC TTCAGTCAGCAGTTATGCAGGGGGTGTCCCTCCCACCCTCAATGAAGGTCTAGAGCTG TTAGATGGGCTCAATCTCACCTCTTCCCATTCCCTGCTATCTCGGAGTGGTCTCTCTGG CTTCTCTTTGCAGCATCCTGGGGTTACCGGCCCCTTACACACCTACAGCAGCTCCCTTT TCAGCCCAGCAGAGGGGCCCCTGTCAGCAGGAGAAGGGTGCTTCTCCAGCTCCCAGG CTCTGGAGGCCCTGCTCACCTCTGATACGCCACCACCCCCTGCTGACGTCCTCATGAC CCAGGTAGATCCCATTCTGTCCCAGGCTCCGACTCTTCTGTTGCTGGGGGGGCTTCCT TCCTCCAGTAAGCTGGCCACGGGCGTCGGCCTGTGTCCCAAGCCCCTAGAGGCTCCA GGCCCCAGCAGTCTGGTTCCCACCCTTTCTATGATAGCACCACCTCCAGTCATGGCAA GTGCCCCCATCCCCAAGGCTCTGGGGACTCCTGTGCTCACACCCCCTACTGAAGCTGC AAGCCAAGACAGAATGCCTCAGGATCTAGATCTTGATATGTATATGGAGAACCTGGAGT GTGACATGGATAACATCATCAGTGACCTCATGGATGAGGGCGAGGGACTGGACTTCAA CTTTGAGCCAGATCCC (SEQ ID NO:62; NM_005938), or a nucleic acid sequence that hybridizes to a nucleic acid sequence consisting of SEQ ID NO:62 under stringent hybridization conditions.

In some embodiments, Forkhead Box 06 (FOXO6) comprises the amino acid sequence: MAAKLRAHQVDVDPDFAPQSRPRSCTWPLPQPDLAGDEDGALGAGVAEGAEDCGPERR ATAPAMAPAPPLGAEVGPLRKAKSSRRNAWGNLSYADLITKAIESAPDKRLTLSQIYDWMV RYVPYFKDKGDSNSSAGWKNSIRHNLSLHTRFIRVQNEGTGKSSWWMLNPEGGKTGKTP RRRAVSMDNGAKFLRIKGKASKKKQLQAPERSPDDSSPSAPAPGPVPAAAKWAASPASHA SDDYEAWADFRGGGRPLLGEAAELEDDEALEALAPSSPLMYPSPASALSPALGSRCPGEL PRLAELGGPLGLHGGGGAGLPEGLLDGAQDAYGPRAAPRPGPVLGAPGELALAGAAAAY PGKGAAPYAPPAPSRSALAHPISLMTLPGEAGAAGLAPPGHAAAFGGPPGGLLLDALPGPY AAAAAGPLGAAPDRFPADLDLDMFSGSLECDVESIILNDFMDSDEMDFNFDSALPPPPPGL AGAPPPNQSWVPG (SEQ ID NO:63; NP_001278210) or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:63.

In some embodiments, the nucleic acid sequence encoding FOXO6 comprises the nucleic acid sequence: ATGGCTGCGAAGCTGCGAGCGCATCAGGTGGACGTGGACCCGGACTTCGCGCCGCA GAGCCGGCCGCGCTCGTGTACCTGGCCCCTGCCGCAGCCTGACTTGGCCGGCGACG AGGACGGAGCGCTGGGCGCAGGGGTGGCCGAGGGCGCCGAGGACTGCGGGCCGGA GCGCCGGGCTACGGCCCCGGCGATGGCCCCAGCGCCGCCCCTGGGCGCGGAGGTC GGACCGCTGCGGAAAGCGAAGAGCTCTCGGCGGAACGCGTGGGGGAACCTGTCCTA CGCCGACCTCATCACCAAAGCCATCGAGAGCGCCCCGGACAAGCGGCTCACGCTCTC GCAGATCTACGACTGGATGGTCCGTTACGTGCCCTACTTCAAGGATAAAGGCGACAGC AACAGCTCGGCCGGCTGGAAGAACTCCATCCGGCACAACCTGTCGCTGCACACCCGT TTCATCCGCGTGCAGAACGAGGGCACCGGCAAGAGTTCGTGGTGGATGCTGAACCCC GAGGGCGGAAAGACAGGGAAGACCCCGCGGCGCAGGGCCGTGTCCATGGACAACGG GGCCAAGTTCCTGCGCATCAAGGGCAAGGCGAGCAAGAAGAAGCAGCTGCAGGCGCC CGAGCGAAGCCCGGACGACAGCTCCCCGAGTGCGCCCGCCCCGGGGCCGGTGCCTG CCGCAGCCAAGTGGGCCGCCAGCCCCGCCTCGCACGCCAGCGACGACTACGAGGCT TGGGCCGACTTCCGCGGCGGCGGGAGACCCCTGCTCGGGGAGGCGGCCGAGCTGGA GGACGACGAGGCCCTGGAGGCCCTGGCGCCATCATCGCCGCTCATGTACCCAAGCCC CGCCAGCGCGCTGTCGCCGGCGCTGGGCTCGCGCTGTCCGGGTGAGCTGCCCCGCC TGGCCGAGCTGGGAGGCCCGCTGGGCCTGCACGGCGGCGGCGGCGCGGGGCTGCC CGAGGGCCTGCTGGACGGCGCGCAGGACGCGTACGGGCCGCGGGCCGCGCCCAGG CCCGGCCCGGTGCTGGGTGCGCCGGGGGAGCTGGCGCTGGCGGGCGCAGCCGCCG CCTACCCCGGCAAAGGGGCGGCCCCGTACGCGCCGCCCGCGCCCTCGCGCAGTGCC TTAGCCCACCCCATCAGCCTTATGACGCTGCCCGGCGAGGCGGGCGCCGCGGGCCT GGCACCGCCGGGCCACGCCGCCGCCTTCGGGGGCCCGCCCGGCGGCCTCCTGCTG GACGCTCTGCCGGGGCCCTACGCTGCCGCCGCCGCCGGGCCGCTGGGCGCCGCGC CCGACCGCTTCCCGGCCGACCTGGACCTCGACATGTTCAGCGGGAGCCTCGAGTGCG ACGTGGAGTCCATCATCCTCAACGACTTCATGGACAGCGACGAAATGGACTTCAACTTC GATTCGGCCCTGCCTCCGCCGCCGCCGGGCCTGGCCGGGGCCCCGCCCCCCAACCA GAGCTGGGTGCCGGGC (SEQ ID NO:64; NM_001291281), or a nucleic acid sequence that hybridizes to a nucleic acid sequence consisting of SEQ ID NO:64 under stringent hybridization conditions.

In some embodiments, Forkhead box P1 (FOXP1) comprises the amino acid sequence: MMQESGTETKSNGSAIQNGSGGSNHLLECGGLREGRSNGETPAVDIGAADLAHAQQQQQ QALQVARQLLLQQQQQQQVSGLKSPKRNDKQPALQVPVSVAMMTPQVITPQQMQQILQQ QVLSPQQLQVLLQQQQALMLQQQQLQEFYKKQQEQLQLQLLQQQHAGKQPKEQQQVAT QQLAFQQQLLQMQQLQQQHLLSLQRQGLLTIQPGQPALPLQPLAQGMIPTELQQLWKEVT SAHTAEETTGNNHSSLDLTTTCVSSSAPSKTSLIMNPHASTNGQLSVHTPKRESLSHEEHP HSHPLYGHGVCKWPGCEAVCEDFQSFLKHLNSEHALDDRSTAQCRVQMQVVQQLELQLA KDKERLQAMMTHLHVKSTEPKAAPQPLNLVSSVTLSKSASEASPQSLPHTPTTPTAPLTPV TQGPSVITTTSMHTVGPIRRRYSDKYNVPISSADIAQNQEFYKNAEVRPPFTYASLIRQAILE SPEKQLTLNEIYNWFTRMFAYFRRNAATWKNAVRHNLSLHKCFVRVENVKGAVWTVDEVE FQKRRPQKISGNPSLIKNMQSSHAYCTPLSAALQASMAENSIPLYTTASMGNPTLGNLASAI REELNGAMEHTNSNESDSSPGRSPMQAVHPVHVKEEPLDPEEAEGPLSLVTTANHSPDFD HDRDYEDEPVNEDME (SEQ ID NO:65; NP_116071), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:65.

In some embodiments, the nucleic acid sequence encoding FOXP1 comprises the nucleic acid sequence: ATGATGCAAGAATCTGGGACTGAGACAAAAAGTAACGGTTCAGCCATCCAGAATGGGT CGGGCGGCAGCAACCACTTACTAGAGTGCGGCGGTCTTCGGGAGGGGCGGTCCAAC GGAGAGACGCCGGCCGTGGACATCGGGGCAGCTGACCTCGCCCACGCCCAGCAGCA GCAGCAACAGGCACTTCAGGTGGCAAGACAGCTCCTTCTTCAGCAGCAACAGCAGCAG CAAGTTAGTGGATTAAAATCTCCCAAGAGGAATGACAAACAACCAGCTCTTCAGGTTCC CGTGTCAGTGGCTATGATGACACCTCAAGTTATCACTCCCCAGCAAATGCAGCAGATC CTCCAGCAACAAGTGCTGAGCCCTCAGCAGCTCCAGGTTCTCCTCCAGCAGCAGCAG GCCCTCATGCTTCAACAGCAGCAGCTTCAAGAGTTTTATAAAAAACAACAGGAACAGTT GCAGCTTCAACTTTTACAACAACAACATGCTGGAAAACAGCCTAAAGAGCAACAGCAGG TGGCTACCCAGCAGTTGGCTTTTCAGCAGCAGCTTTTACAGATGCAGCAGTTACAGCA GCAGCACCTCCTGTCTTTGCAGCGCCAAGGCCTTCTGACAATTCAGCCCGGGCAGCCT GCCCTTCCCCTTCAACCTCTTGCTCAAGGCATGATTCCAACAGAACTGCAGCAGCTCTG GAAAGAAGTGACAAGTGCTCATACTGCAGAAGAAACCACAGGCAACAATCACAGCAGT TTGGATCTGACCACGACATGTGTCTCCTCCTCTGCACCTTCCAAGACCTCCTTAATAAT GAACCCACATGCCTCTACCAATGGACAGCTCTCAGTCCACACTCCCAAAAGGGAAAGT TTGTCCCATGAGGAGCACCCCCATAGCCATCCTCTCTATGGACATGGTGTATGCAAGT GGCCAGGCTGTGAAGCAGTGTGCGAAGATTTCCAATCATTTCTAAAACATCTCAACAGT GAGCATGCGCTGGACGATAGAAGTACAGCCCAATGTAGAGTACAAATGCAGGTTGTAC AGCAGTTAGAGCTACAGCTTGCAAAAGACAAAGAACGCCTGCAAGCCATGATGACCCA CCTGCATGTGAAGTCTACAGAACCCAAAGCCGCCCCTCAGCCCTTGAATCTGGTATCA AGTGTCACTCTCTCCAAGTCCGCATCGGAGGCTTCTCCACAGAGCTTACCTCATACTCC AACGACCCCAACCGCCCCCCTGACTCCCGTCACCCAAGGCCCCTCTGTCATCACAACC ACCAGCATGCACACGGTGGGACCCATCCGCAGGCGGTACTCAGACAAATACAACGTG CCCATTTCGTCAGCAGATATTGCGCAGAACCAAGAATTTTATAAGAACGCAGAAGTTAG ACCACCATTTACATATGCATCTTTAATTAGGCAGGCCATTCTCGAATCTCCAGAAAAGCA GCTAACACTAAATGAGATCTATAACTGGTTCACACGAATGTTTGCTTACTTCCGACGCA ACGCGGCCACGTGGAAGAATGCAGTGCGTCATAATCTTAGTCTTCACAAGTGTTTTGTG CGAGTAGAAAACGTTAAAGGGGCAGTATGGACAGTGGATGAAGTAGAATTCCAAAAAC GAAGGCCACAAAAGATCAGTGGTAACCCTTCCCTTATTAAAAACATGCAGAGCAGCCAC GCCTACTGCACACCTCTCAGTGCAGCTTTACAGGCTTCAATGGCTGAGAATAGTATACC TCTATACACTACCGCTTCCATGGGAAATCCCACTCTGGGCAACTTAGCCAGCGCAATAC GGGAAGAGCTGAACGGGGCAATGGAGCATACCAACAGCAACGAGAGTGACAGCAGTC CAGGCAGATCTCCTATGCAAGCCGTGCATCCTGTACACGTCAAAGAAGAGCCCCTCGA TCCAGAGGAAGCTGAAGGGCCCCTGTCCTTAGTGACAACAGCCAACCACAGTCCAGAT TTTGACCATGACAGAGATTACGAAGATGAACCAGTAAACGAGGACATGGAG (SEQ ID NO:66; NM_032682), or a nucleic acid sequence that hybridizes to a nucleic acid sequence consisting of SEQ ID NO:66 under stringent hybridization conditions.

In some embodiments, Forkhead box P2 (FOXP2) comprises the amino acid sequence MMQESATETISNSSMNQNGMSTLSSQLDAGSRDGRSSGDTSSEVSTVELLHLQQQQALQ AARQLLLQQQTSGLKSPKSSDKQRPLQVPVSVAMMTPQVITPQQMQQILQQQVLSPQQLQ ALLQQQQAVMLQQQQLQEFYKKQQEQLHLQLLQQQQQQQQQQQQQQQQQQQQQQQQ QQQQQQQQQQQQQQQQHPGKQAKEQQQQQQQQQQLAAQQLVFQQQLLQMQQLQQQ QHLLSLQRQGLISIPPGQAALPVQSLPQAGLSPAEIQQLWKEVTGVHSMEDNGIKHGGLDL TTNNSSSTTSSNTSKASPPITHHSIVNGQSSVLSARRDSSSHEETGASHTLYGHGVCKWPG CESICEDFGQFLKHLNNEHALDDRSTAQCRVQMQVVQQLEIQLSKERERLQAMMTHLHMR PSEPKPSPKPLNLVSSVTMSKNMLETSPQSLPQTPTTPTAPVTPITQGPSVITPASVPNVGA IRRRHSDKYNIPMSSEIAPNYEFYKNADVRPPFTYATLIRQAIMESSDRQLTLNEIYSWFTRT FAYFRRNAATWKNAVRHNLSLHKCFVRVENVKGAVWTVDEVEYQKRRSQKITGSPTLVKN IPTSLGYGAALNASLQAALAESSLPLLSNPGLINNASSGLLQAVHEDLNGSLDHIDSNGNSS PGCSPQPHIHSIHVKEEPVIAEDEDCPMSLVTTANHSPELEDDREIEEEPLSEDLE (SEQ ID NO:67; NP_055306), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:67.

In some embodiments, the nucleic acid sequence encoding FOXP2 comprises the nucleic acid sequence: ATGATGCAGGAATCTGCGACAGAGACAATAAGCAACAGTTCAATGAATCAAAATGGAAT GAGCACTCTAAGCAGCCAATTAGATGCTGGCAGCAGAGATGGAAGATCAAGTGGTGAC ACCAGCTCTGAAGTAAGCACAGTAGAACTGCTGCATCTGCAACAACAGCAGGCTCTCC AGGCAGCAAGACAACTTCTTTTACAGCAGCAAACAAGTGGATTGAAATCTCCTAAGAGC AGTGATAAACAGAGACCACTGCAGGTGCCTGTGTCAGTGGCCATGATGACTCCCCAGG TGATCACCCCTCAGCAAATGCAGCAGATCCTTCAGCAACAAGTCCTGTCTCCTCAGCA GCTACAAGCCCTTCTCCAACAACAGCAGGCTGTCATGCTGCAGCAGCAACAACTACAA GAGTTTTACAAGAAACAGCAAGAGCAGTTACATCTTCAGCTTTTGCAGCAGCAGCAGCA ACAGCAGCAGCAGCAACAACAGCAGCAACAACAGCAGCAGCAACAACAACAACAACAG CAGCAACAACAGCAGCAGCAGCAGCAACAGCAGCAGCAGCAGCAACAGCATCCTGGA AAGCAAGCGAAAGAGCAGCAGCAGCAGCAGCAGCAGCAACAGCAATTGGCAGCCCAG CAGCTTGTCTTCCAGCAGCAGCTTCTCCAGATGCAACAACTCCAGCAGCAGCAGCATC TGCTCAGCCTTCAGCGTCAGGGACTCATCTCCATTCCACCTGGCCAGGCAGCACTTCC TGTCCAATCGCTGCCTCAAGCTGGCTTAAGTCCTGCTGAGATTCAGCAGTTATGGAAAG AAGTGACTGGAGTTCACAGTATGGAAGACAATGGCATTAAACATGGAGGGCTAGACCT CACTACTAACAATTCCTCCTCGACTACCTCCTCCAACACTTCCAAAGCATCACCACCAA TAACTCATCATTCCATAGTGAATGGACAGTCTTCAGTTCTAAGTGCAAGACGAGACAGC TCGTCACATGAGGAGACTGGGGCCTCTCACACTCTCTATGGCCATGGAGTTTGCAAAT GGCCAGGCTGTGAAAGCATTTGTGAAGATTTTGGACAGTTTTTAAAGCACCTTAACAAT GAACACGCATTGGATGACCGAAGCACTGCTCAGTGTCGAGTGCAAATGCAGGTGGTGC AACAGTTAGAAATACAGCTTTCTAAAGAACGCGAACGTCTTCAAGCAATGATGACCCAC TTGCACATGCGACCCTCAGAGCCCAAACCATCTCCCAAACCTCTAAATCTGGTGTCTAG TGTCACCATGTCGAAGAATATGTTGGAGACATCCCCACAGAGCTTACCTCAAACCCCTA CCACACCAACGGCCCCAGTCACCCCGATTACCCAGGGACCCTCAGTAATCACCCCAGC CAGTGTGCCCAATGTGGGAGCCATACGAAGGCGACATTCAGACAAATACAACATTCCC ATGTCATCAGAAATTGCCCCAAACTATGAATTTTATAAAAATGCAGATGTCAGACCTCCA TTTACTTATGCAACTCTCATAAGGCAGGCTATCATGGAGTCATCTGACAGGCAGTTAAC ACTTAATGAAATTTACAGCTGGTTTACACGGACATTTGCTTACTTCAGGCGTAATGCAG CAACTTGGAAGAATGCAGTACGTCATAATCTTAGCCTGCACAAGTGTTTTGTTCGAGTA GAAAATGTTAAAGGAGCAGTATGGACTGTGGATGAAGTAGAATACCAGAAGCGAAGGT CACAAAAGATAACAGGAAGTCCAACCTTAGTAAAAAATATACCTACCAGTTTAGGCTAT GGAGCAGCTCTTAATGCCAGTTTGCAGGCTGCCTTGGCAGAGAGCAGTTTACCTTTGC TAAGTAATCCTGGACTGATAAATAATGCATCCAGTGGCCTACTGCAGGCCGTCCACGAA GACCTCAATGGTTCTCTGGATCACATTGACAGCAATGGAAACAGTAGTCCGGGCTGCT CACCTCAGCCGCACATACATTCAATCCACGTCAAGGAAGAGCCAGTGATTGCAGAGGA TGAAGACTGCCCAATGTCCTTAGTGACAACAGCTAATCACAGTCCAGAATTAGAAGACG ACAGAGAGATTGAAGAAGAGCCTTTATCTGAAGATCTGGAA (SEQ ID NO:68; NM_014491), or a nucleic acid sequence that hybridizes to a nucleic acid sequence consisting of SEQ ID NO:68 under stringent hybridization conditions.

In some embodiments, Forkhead box P3 (FOXP3) comprises the amino acid sequence MPNPRPGKPSAPSLALGPSPGASPSWRAAPKASDLLGARGPGGTFQGRDLRGGAHASSS SLNPMPPSQLQLPTLPLVMVAPSGARLGPLPHLQALLQDRPHFMHQLSTVDAHARTPVLQ VHPLESPAMISLTPPTTATGVFSLKARPGLPPGINVASLEWVSREPALLCTFPNPSAPRKDS TLSAVPQSSYPLLANGVCKWPGCEKVFEEPEDFLKHCQADHLLDEKGRAQCLLQREMVQ SLEQQLVLEKEKLSAMQAHLAGKMALTKASSVASSDKGSCCIVAAGSQGPVVPAWSGPRE APDSLFAVRRHLWGSHGNSTFPEFLHNMDYFKFHNMRPPFTYATLIRWAILEAPEKQRTLN EIYHWFTRMFAFFRNHPATWKNAIRHNLSLHKCFVRVESEKGAVWTVDELEFRKKRSQRP SRCSNPTPGP (SEQ ID NO:69; NP_054728), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:69.

In some embodiments, the nucleic acid sequence encoding FOXP3 comprises the nucleic acid sequence: ATGCCCAACCCCAGGCCTGGCAAGCCCTCGGCCCCTTCCTTGGCCCTTGGCCCATCC CCAGGAGCCTCGCCCAGCTGGAGGGCTGCACCCAAAGCCTCAGACCTGCTGGGGGC CCGGGGCCCAGGGGGAACCTTCCAGGGCCGAGATCTTCGAGGCGGGGCCCATGCCT CCTCTTCTTCCTTGAACCCCATGCCACCATCGCAGCTGCAGCTGCCCACACTGCCCCT AGTCATGGTGGCACCCTCCGGGGCACGGCTGGGCCCCTTGCCCCACTTACAGGCACT CCTCCAGGACAGGCCACATTTCATGCACCAGCTCTCAACGGTGGATGCCCACGCCCG GACCCCTGTGCTGCAGGTGCACCCCCTGGAGAGCCCAGCCATGATCAGCCTCACACC ACCCACCACCGCCACTGGGGTCTTCTCCCTCAAGGCCCGGCCTGGCCTCCCACCTGG GATCAACGTGGCCAGCCTGGAATGGGTGTCCAGGGAGCCGGCACTGCTCTGCACCTT CCCAAATCCCAGTGCACCCAGGAAGGACAGCACCCTTTCGGCTGTGCCCCAGAGCTC CTACCCACTGCTGGCAAATGGTGTCTGCAAGTGGCCCGGATGTGAGAAGGTCTTCGAA GAGCCAGAGGACTTCCTCAAGCACTGCCAGGCGGACCATCTTCTGGATGAGAAGGGC AGGGCACAATGTCTCCTCCAGAGAGAGATGGTACAGTCTCTGGAGCAGCAGCTGGTG CTGGAGAAGGAGAAGCTGAGTGCCATGCAGGCCCACCTGGCTGGGAAAATGGCACTG ACCAAGGCTTCATCTGTGGCATCATCCGACAAGGGCTCCTGCTGCATCGTAGCTGCTG GCAGCCAAGGCCCTGTCGTCCCAGCCTGGTCTGGCCCCCGGGAGGCCCCTGACAGC CTGTTTGCTGTCCGGAGGCACCTGTGGGGTAGCCATGGAAACAGCACATTCCCAGAGT TCCTCCACAACATGGACTACTTCAAGTTCCACAACATGCGACCCCCTTTCACCTACGCC ACGCTCATCCGCTGGGCCATCCTGGAGGCTCCAGAGAAGCAGCGGACACTCAATGAG ATCTACCACTGGTTCACACGCATGTTTGCCTTCTTCAGAAACCATCCTGCCACCTGGAA GAACGCCATCCGCCACAACCTGAGTCTGCACAAGTGCTTTGTGCGGGTGGAGAGCGA GAAGGGGGCTGTGTGGACCGTGGATGAGCTGGAGTTCCGCAAGAAACGGAGCCAGAG GCCCAGCAGGTGTTCCAACCCTACACCTGGCCCC (SEQ ID NO:70; NM_014009), or a nucleic acid sequence that hybridizes to a nucleic acid sequence consisting of SEQ ID NO:70 under stringent hybridization conditions.

In some embodiments, Forkhead box 4 (FOXP4) comprises the amino acid sequence: MMVESASETIRSAPSGQNGVGSLSGQADGSSGGATGTTASGTGREVTTGADSNGEMSPA ELLHFQQQQALQVARQFLLQQASGLSSPGNNDSKQSASAVQVPVSVAMMSPQMLTPQQ MQQILSPPQLQALLQQQQALMLQQEYYKKQQEQLHLQLLTQQQAGKPQPKEALGNKQLA FQQQLLQMQQLQQQHLLNLQRQGLVSLQPNQASGPLQTLPQAAVCPTDLPQLWKGEGAP GQPAEDSVKQEGLDLTGTAATATSFAAPPKVSPPLSHHTLPNGQPTVLTSRRDSSSHEETP GSHPLYGHGECKWPGCETLCEDLGQFIKHLNTEHALDDRSTAQCRVQMQVVQQLEIQLAK ESERLQAMMAHLHMRPSEPKPFSQPLNPVPGSSSFSKVTVSAADSFPDGLVHPPTSAAAP VTPLRPPGLGSASLHGGGPARRRSSDKFCSPISSELAQNHEFYKNADVRPPFTYASLIRQAI LETPDRQLTLNEIYNWFTRMFAYFRRNTATWKNAVRHNLSLHKCFVRVENVKGAVWTVDE REYQKRRPPKMTGSPTLVKNMISGLSYGALNASYQAALAESSFPLLNSPGMLNPGSASSLL PLSHDDVGAPVEPLPSNGSSSPPRLSPPQYSHQVQVKEEPAEAEEDRQPGPPLGAPNPS ASGPPEDRDLEEELPGEELS (SEQ ID NO:71 NP_001012426), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:71.

In some embodiments, the nucleic acid sequence encoding FOXP4 comprises the nucleic acid sequence: ATGATGGTGGAATCTGCCTCGGAGACAATCAGGTCGGCTCCATCTGGTCAGAATGGCG TGGGCAGCCTCTCTGGGCAAGCAGATGGCAGCAGCGGCGGGGCCACAGGGACAACT GCAAGTGGCACGGGCAGGGAAGTGACCACGGGTGCAGACAGCAATGGTGAGATGAGT CCCGCAGAGCTGCTGCACTTCCAGCAGCAACAGGCTCTCCAAGTGGCCCGGCAGTTC CTGCTGCAGCAGGCCTCAGGCCTGAGCTCCCCAGGGAACAATGACAGCAAACAGTCT GCCTCTGCTGTGCAGGTGCCTGTGTCGGTGGCCATGATGTCGCCGCAGATGCTTACC CCGCAACAGATGCAGCAGATCCTGTCGCCCCCGCAGCTGCAGGCCTTGCTCCAGCAG CAGCAAGCCCTCATGCTCCAGCAGGAGTACTACAAGAAGCAGCAGGAGCAGCTCCAC CTGCAGCTCCTCACCCAGCAGCAGGCTGGGAAACCGCAGCCCAAAGAGGCACTGGGG AACAAGCAGCTGGCCTTCCAGCAGCAGCTCCTGCAAATGCAACAGTTGCAGCAGCAGC ACCTGCTCAACCTGCAGAGGCAGGGGCTGGTCAGCCTGCAGCCCAACCAAGCCTCGG GGCCCCTCCAGACCCTTCCGCAAGCAGCTGTTTGCCCAACAGACCTGCCCCAGCTGT GGAAGGGCGAGGGTGCCCCCGGGCAGCCTGCCGAGGACAGCGTCAAGCAGGAGGG GCTGGACCTCACTGGCACGGCCGCCACCGCTACCTCGTTTGCCGCTCCCCCCAAGGT CTCACCCCCCCTCTCCCACCATACCCTGCCCAACGGACAGCCTACTGTGCTCACATCT CGGAGAGACAGCTCTTCCCACGAGGAGACCCCCGGCTCCCACCCCCTGTACGGACAC GGAGAGTGCAAGTGGCCAGGCTGTGAGACCCTGTGTGAAGACCTGGGCCAGTTTATC AAACACCTCAACACAGAGCACGCCCTGGATGACCGGAGTACAGCCCAGTGCCGGGTA CAGATGCAGGTGGTGCAGCAGCTGGAGATCCAGCTCGCCAAGGAGAGCGAGCGGCT GCAGGCCATGATGGCCCACCTGCACATGCGGCCCTCGGAGCCCAAGCCCTTCAGCCA GCCACTGAACCCGGTCCCCGGCTCCTCCTCATTCTCCAAGGTGACCGTCTCTGCAGCA GACTCATTCCCAGATGGTCTCGTGCACCCCCCGACCTCGGCCGCAGCCCCTGTCACC CCTCTACGGCCCCCTGGCCTGGGCTCTGCCTCCCTGCATGGTGGGGGCCCAGCCCGT CGGAGAAGCAGTGACAAGTTCTGCTCCCCCATCTCCTCAGAGCTGGCCCAGAATCATG AGTTCTACAAGAACGCCGACGTCCGGCCCCCCTTCACCTACGCCTCCCTCATCCGCCA GGCCATCCTGGAAACCCCTGACAGGCAGCTGACCCTGAATGAGATCTATAACTGGTTC ACCAGGATGTTCGCCTATTTCCGCAGAAACACTGCCACCTGGAAGAACGCCGTGCGCC ACAACCTCAGCCTGCACAAGTGCTTCGTCCGCGTGGAGAACGTCAAGGGTGCCGTGT GGACTGTGGACGAGCGGGAGTATCAGAAGCGGAGACCGCCAAAGATGACAGGGAGC CCCACCCTGGTGAAGAACATGATCTCTGGCCTCAGCTATGGAGCACTTAATGCCAGCT ACCAGGCCGCCCTGGCCGAGAGCAGCTTCCCCCTCCTCAACAGCCCTGGCATGCTGA ACCCTGGCTCCGCCAGCAGCCTGCTGCCCCTCAGCCACGATGACGTGGGTGCCCCCG TGGAGCCGCTGCCCAGCAACGGCAGCAGCAGCCCTCCTCGCCTCTCCCCGCCCCAGT ACAGCCACCAGGTGCAGGTGAAGGAGGAGCCAGCAGAGGCAGAGGAAGACAGGCAG CCCGGGCCTCCCCTGGGCGCCCCTAACCCCAGCGCCTCGGGGCCTCCGGAAGACAG GGACCTGGAGGAGGAGCTGCCGGGAGAAGAACTGTCC (SEQ ID NO:72; NM_001012426), or a nucleic acid sequence that hybridizes to a nucleic acid sequence consisting of SEQ ID NO:72 under stringent hybridization conditions.

In some embodiments, Forkhead box Q1 (FOXQ1) comprises the amino acid sequence: MKLEVFVPRAAHGDKQGSDLEGAGGSDAPSPLSAAGDDSLGSDGDCAANSPAAGGGAR DPPGDGEQSAGGGPGAEEAIPAAAAAAVVAEGAEAGAAGPGAGGAGSGEGARSKPYTR RPKPPYSYIALIAMAIRDSAGGRLTLAEINEYLMGKFPFFRGSYTGWRNSVRHNLSLNDCFV KVLRDPSRPWGKDNYWMLNPNSEYTFADGVFRRRRKRLSHRAPVPAPGLRPEEAPGLPA APPPAPAAPASPRMRSPARQEERASPAGKFSSSFAIDSILRKPFRSRRLRDTAPGTTLQWG AAPCPPLPAFPALLPAAPCRALLPLCAYGAGEPARLGAREAEVPPTAPPLLLAPLPAAAPAK PLRGPAAGGAHLYCPLRLPAALQAASVRRPGPHLPYPVETLLA (SEQ ID NO:73; NP_150285), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:73.

In some embodiments, the nucleic acid sequence encoding FOXQ1 comprises the nucleic acid sequence: ATGAAGTTGGAGGTGTTCGTCCCTCGCGCGGCCCACGGGGACAAGCAGGGCAGTGAC CTGGAGGGCGCGGGCGGCAGCGACGCGCCGTCCCCGCTGTCGGCGGCGGGAGACG ACTCCCTGGGCTCAGATGGGGACTGCGCGGCCAACAGCCCGGCCGCGGGCGGCGGC GCCAGAGATCCGCCGGGCGACGGCGAACAGAGTGCGGGAGGCGGGCCGGGCGCGG AGGAGGCGATCCCGGCAGCAGCTGCTGCAGCGGTGGTGGCGGAGGGCGCGGAGGC CGGGGCGGCGGGGCCAGGCGCGGGCGGCGCGGGGAGCGGCGAGGGTGCACGCAG CAAGCCATATACGCGGCGGCCCAAGCCCCCCTACTCGTACATCGCGCTCATCGCCATG GCCATCCGCGACTCGGCGGGCGGGCGCTTGACGCTGGCGGAGATCAACGAGTACCT CATGGGCAAGTTCCCCTTTTTCCGCGGCAGCTACACGGGCTGGCGCAACTCCGTGCG CCACAACCTTTCGCTCAACGACTGCTTCGTCAAGGTGCTGCGCGACCCCTCGCGGCCC TGGGGCAAGGACAACTACTGGATGCTCAACCCCAACAGCGAGTACACCTTCGCCGAC GGGGTCTTCCGCCGCCGCCGCAAGCGCCTCAGCCACCGCGCGCCGGTCCCCGCGCC CGGGCTGCGGCCCGAGGAGGCCCCGGGCCTCCCCGCCGCCCCGCCGCCCGCGCCC GCCGCCCCGGCCTCGCCCCGCATGCGCTCGCCCGCCCGCCAGGAGGAGCGCGCCAG CCCCGCGGGCAAGTTCTCCAGCTCCTTCGCCATCGACAGCATCCTGCGCAAGCCCTTC CGCAGCCGCCGCCTCAGGGACACGGCCCCCGGGACGACGCTTCAGTGGGGCGCCGC GCCCTGCCCGCCGCTGCCCGCGTTCCCCGCGCTCCTCCCCGCGGCGCCCTGCAGGG CCCTGCTGCCGCTCTGCGCGTACGGCGCGGGCGAGCCGGCGCGGCTGGGCGCGCG CGAGGCCGAGGTGCCACCGACCGCGCCGCCCCTCCTGCTTGCACCTCTCCCGGCGG CGGCCCCCGCCAAGCCACTCCGAGGCCCGGCGGCCGGCGGCGCGCACCTGTACTGC CCCCTGCGGCTGCCCGCAGCCCTGCAGGCGGCCTCAGTCCGCCGCCCTGGCCCGCA CCTGCCGTACCCGGTGGAGACGCTCCTAGCC (SEQ ID NO:74; NM_033260), or a nucleic acid sequence that hybridizes to a nucleic acid sequence consisting of SEQ ID NO:74 under stringent hybridization conditions.

In some embodiments, Forkhead Box R1 (FOXR1) comprises the amino acid sequence: MGNELFLAFTTSHLPLAEQKLARYKLRIVKPPKLPLEKKPNPDKDGPDYEPNLWMWVNPNI VYPPGKLEVSGRRKREDLTSTLPSSQPPQKEEDASCSEAAGVESLSQSSSKRSPPRKRFA FSPSTWELTEEEEAEDQEDSSSMALPSPHKRAPLQSRRLRQASSQAGRLWSRPPLNYFHL IALALRNSSPCGLNVQQIYSFTRKHFPFFRTAPEGWKNTVRHNLCFRDSFEKVPVSMQGG ASTRPRSCLWKLTEEGHRRFAEEARALASTRLESIQQCMSQPDVMPFLFDL (SEQ ID NO:75; NP_859072), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:75.

In some embodiments, the nucleic acid sequence encoding FOXR1 comprises the nucleic acid sequence: ATGGGGAACGAGCTCTTTCTGGCCTTCACCACATCTCACCTCCCCTTAGCGGAGCAGA AACTTGCCAGGTATAAACTCCGAATTGTTAAGCCACCAAAATTACCCCTAGAGAAAAAA CCCAACCCTGATAAGGATGGTCCAGATTATGAGCCCAACCTCTGGATGTGGGTAAATC CCAACATTGTGTATCCCCCTGGAAAGCTGGAGGTCTCAGGACGTAGGAAGAGGGAGG ACCTGACAAGCACACTCCCCTCCTCTCAGCCACCCCAGAAGGAGGAAGATGCCAGCTG CTCAGAGGCCGCAGGGGTGGAATCACTGTCCCAGTCCTCCAGCAAGCGGTCTCCCCC TCGGAAGCGGTTTGCCTTTTCCCCCAGCACCTGGGAGCTCACAGAAGAGGAGGAGGC TGAGGACCAGGAAGACAGCTCCTCTATGGCTCTCCCATCCCCTCACAAAAGGGCCCCC CTCCAGAGTCGGAGGCTTCGGCAAGCCAGCAGCCAGGCGGGGAGGCTCTGGTCCCG GCCCCCTCTCAATTACTTCCACCTAATTGCCCTGGCATTAAGAAACAGTTCCCCCTGTG GCCTCAACGTGCAACAGATCTACAGTTTCACTCGAAAGCACTTCCCCTTTTTCCGGACG GCCCCGGAAGGCTGGAAGAATACTGTCCGTCACAATCTCTGTTTTCGAGACAGCTTTG AGAAAGTGCCTGTCAGCATGCAGGGCGGGGCCAGCACACGGCCTCGATCTTGCCTCT GGAAGTTGACCGAGGAGGGACACCGCCGCTTTGCGGAGGAGGCCCGCGCCTTGGCT TCCACTCGGCTAGAAAGTATCCAACAGTGCATGAGCCAGCCAGATGTGATGCCCTTCC TCTTTGATCTT (SEQ ID NO:76; NM_181721), or a nucleic acid sequence that hybridizes to a nucleic acid sequence consisting of SEQ ID NO:76 under stringent hybridization conditions.

In some embodiments, FOXR2 comprises the amino acid sequence MDLKLKDCEFWYSLHGQVPGLLDWDMRNELFLPCTTDQCSLAEQILAKYRVGVMKPPEM PQKRRPSPDGDGPPCEPNLWMWVDPNILCPLGSQEAPKPSGKEDLTNISPFPQPPQKDE GSNCSEDKVVESLPSSSSEQSPLQKQGIHSPSDFELTEEEAEEPDDNSLQSPEMKCYQSQ KLWQINNQEKSWQRPPLNCSHLIALALRNNPHCGLSVQEIYNFTRQHFPFFWTAPDGWKS TIHYNLCFLDSFEKVPDSLKDEDNARPRSCLWKLTKEGHRRFWEETRVLAFAQRERIQECM SQPELLTSLFDL (SEQ ID NO:77; NP_940853), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:77.

In some embodiments, the nucleic acid sequence encoding FOXR2 comprises the nucleic acid sequence: ATGGACTTAAAACTAAAAGACTGTGAATTTTGGTATAGTCTCCATGGCCAGGTCCCAGG GCTGCTGGACTGGGACATGAGGAATGAGTTATTTCTGCCTTGTACCACAGACCAGTGC TCTTTAGCTGAGCAAATCCTTGCCAAATACAGAGTCGGAGTAATGAAGCCCCCAGAAAT GCCTCAGAAGAGGAGACCCAGTCCTGATGGAGATGGTCCTCCCTGTGAACCCAATCTG TGGATGTGGGTGGACCCCAATATCCTGTGCCCCCTTGGCAGCCAGGAGGCCCCAAAG CCCAGTGGAAAAGAGGATCTGACAAACATTTCTCCTTTCCCTCAGCCCCCACAAAAAGA CGAAGGGTCTAACTGCTCAGAGGACAAAGTGGTAGAGTCTCTGCCATCTTCCTCCAGT GAGCAGTCTCCTTTACAGAAGCAGGGTATCCATTCCCCCAGTGACTTTGAGCTCACAG AAGAGGAGGCTGAGGAACCAGACGACAACTCCCTCCAGTCCCCTGAAATGAAATGTTA CCAGAGCCAGAAACTATGGCAAATCAACAACCAAGAGAAGTCCTGGCAAAGGCCCCCT CTCAATTGTAGCCACCTTATTGCCCTAGCATTAAGAAACAACCCCCACTGTGGCCTCAG TGTGCAGGAGATCTACAATTTCACCCGACAGCATTTCCCCTTTTTCTGGACAGCTCCGG ATGGCTGGAAGAGCACCATTCATTACAACCTCTGCTTCCTGGACAGCTTTGAGAAGGT GCCAGACAGCCTTAAGGATGAAGATAATGCAAGACCTCGCTCTTGCCTTTGGAAGCTC ACTAAGGAGGGGCACCGCCGCTTTTGGGAGGAGACTCGTGTCTTAGCCTTTGCTCAAA GGGAGAGAATCCAAGAGTGCATGAGTCAGCCAGAGTTGTTGACCTCTCTCTTTGATCTT (SEQ ID NO:78; NM_198451), or a nucleic acid sequence that hybridizes to a nucleic acid sequence consisting of SEQ ID NO:78 under stringent hybridization conditions.

In some embodiments, Hypoxia inducible factor 1 subunit alpha (HIF-1α) comprises the amino acid sequence: MEGAGGANDKKKISSERRKEKSRDAARSRRSKESEVFYELAHQLPLPHNVSSHLDKASVM RLTISYLRVRKLLDAGDLDIEDDMKAQMNCFYLKALDGFVMVLTDDGDMIYISDNVNKYMGL TQFELTGHSVFDFTHPCDHEEMREMLTHRNGLVKKGKEQNTQRSFFLRMKCTLTSRGRT MNIKSATWKVLHCTGHIHVYDTNSNQPQCGYKKPPMTCLVLICEPIPHPSNIEIPLDSKTFLS RHSLDMKFSYCDERITELMGYEPEELLGRSIYEYYHALDSDHLTKTHHDMFTKGQVTTGQY RMLAKRGGYVWVETQATVIYNTKNSQPQCIVCVNYVVSGIIQHDLIFSLQQTECVLKPVESS DMKMTQLFTKVESEDTSSLFDKLKKEPDALTLLAPAAGDTIISLDFGSNDTETDDQQLEEVP LYNDVMLPSPNEKLQNINLAMSPLPTAETPKPLRSSADPALNQEVALKLEPNPESLELSFTM PQIQDQTPSPSDGSTRQSSPEPNSPSEYCFYVDSDMVNEFKLELVEKLFAEDTEAKNPFST QDTDLDLEMLAPYIPMDDDFQLRSFDQLSPLESSSASPESASPQSTVTVFQQTQIQEPTAN ATTTTATTDELKTVTKDRMEDIKILIASPSPTHIHKETTSATSSPYRDTQSRTASPNRAGKGVI EQTEKSHPRSPNVLSVALSQRTTVPEEELNPKILALQNAQRKRKMEHDGSLFQAVGIGTLL QQPDDHAATTSLSWKRVKGCKSSEQNGMEQKTIILIPSDLACRLLGQSMDESGLPQLTSYD CEVNAPIQGSRNLLQGEELLRALDQVN (SEQ ID NO:79; NP_001521), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:79.

In some embodiments, the nucleic acid sequence encoding HIF-1a comprises the nucleic acid sequence: ATGGAGGGCGCCGGCGGCGCGAACGACAAGAAAAAGATAAGTTCTGAACGTCGAAAA GAAAAGTCTCGAGATGCAGCCAGATCTCGGCGAAGTAAAGAATCTGAAGTTTTTTATGA GCTTGCTCATCAGTTGCCACTTCCACATAATGTGAGTTCGCATCTTGATAAGGCCTCTG TGATGAGGCTTACCATCAGCTATTTGCGTGTGAGGAAACTTCTGGATGCTGGTGATTTG GATATTGAAGATGACATGAAAGCACAGATGAATTGCTTTTATTTGAAAGCCTTGGATGG TTTTGTTATGGTTCTCACAGATGATGGTGACATGATTTACATTTCTGATAATGTGAACAA ATACATGGGATTAACTCAGTTTGAACTAACTGGACACAGTGTGTTTGATTTTACTCATCC ATGTGACCATGAGGAAATGAGAGAAATGCTTACACACAGAAATGGCCTTGTGAAAAAG GGTAAAGAACAAAACACACAGCGAAGCTTTTTTCTCAGAATGAAGTGTACCCTAACTAG CCGAGGAAGAACTATGAACATAAAGTCTGCAACATGGAAGGTATTGCACTGCACAGGC CACATTCACGTATATGATACCAACAGTAACCAACCTCAGTGTGGGTATAAGAAACCACC TATGACCTGCTTGGTGCTGATTTGTGAACCCATTCCTCACCCATCAAATATTGAAATTCC TTTAGATAGCAAGACTTTCCTCAGTCGACACAGCCTGGATATGAAATTTTCTTATTGTGA TGAAAGAATTACCGAATTGATGGGATATGAGCCAGAAGAACTTTTAGGCCGCTCAATTT ATGAATATTATCATGCTTTGGACTCTGATCATCTGACCAAAACTCATCATGATATGTTTA CTAAAGGACAAGTCACCACAGGACAGTACAGGATGCTTGCCAAAAGAGGTGGATATGT CTGGGTTGAAACTCAAGCAACTGTCATATATAACACCAAGAATTCTCAACCACAGTGCA TTGTATGTGTGAATTACGTTGTGAGTGGTATTATTCAGCACGACTTGATTTTCTCCCTTC AACAAACAGAATGTGTCCTTAAACCGGTTGAATCTTCAGATATGAAAATGACTCAGCTAT TCACCAAAGTTGAATCAGAAGATACAAGTAGCCTCTTTGACAAACTTAAGAAGGAACCT GATGCTTTAACTTTGCTGGCCCCAGCCGCTGGAGACACAATCATATCTTTAGATTTTGG CAGCAACGACACAGAAACTGATGACCAGCAACTTGAGGAAGTACCATTATATAATGATG TAATGCTCCCCTCACCCAACGAAAAATTACAGAATATAAATTTGGCAATGTCTCCATTAC CCACCGCTGAAACGCCAAAGCCACTTCGAAGTAGTGCTGACCCTGCACTCAATCAAGA AGTTGCATTAAAATTAGAACCAAATCCAGAGTCACTGGAACTTTCTTTTACCATGCCCCA GATTCAGGATCAGACACCTAGTCCTTCCGATGGAAGCACTAGACAAAGTTCACCTGAG CCTAATAGTCCCAGTGAATATTGTTTTTATGTGGATAGTGATATGGTCAATGAATTCAAG TTGGAATTGGTAGAAAAACTTTTTGCTGAAGACACAGAAGCAAAGAACCCATTTTCTACT CAGGACACAGATTTAGACTTGGAGATGTTAGCTCCCTATATCCCAATGGATGATGACTT CCAGTTACGTTCCTTCGATCAGTTGTCACCATTAGAAAGCAGTTCCGCAAGCCCTGAAA GCGCAAGTCCTCAAAGCACAGTTACAGTATTCCAGCAGACTCAAATACAAGAACCTACT GCTAATGCCACCACTACCACTGCCACCACTGATGAATTAAAAACAGTGACAAAAGACCG TATGGAAGACATTAAAATATTGATTGCATCTCCATCTCCTACCCACATACATAAAGAAAC TACTAGTGCCACATCATCACCATATAGAGATACTCAAAGTCGGACAGCCTCACCAAACA GAGCAGGAAAAGGAGTCATAGAACAGACAGAAAAATCTCATCCAAGAAGCCCTAACGT GTTATCTGTCGCTTTGAGTCAAAGAACTACAGTTCCTGAGGAAGAACTAAATCCAAAGA TACTAGCTTTGCAGAATGCTCAGAGAAAGCGAAAAATGGAACATGATGGTTCACTTTTT CAAGCAGTAGGAATTGGAACATTATTACAGCAGCCAGACGATCATGCAGCTACTACATC ACTTTCTTGGAAACGTGTAAAAGGATGCAAATCTAGTGAACAGAATGGAATGGAGCAAA AGACAATTATTTTAATACCCTCTGATTTAGCATGTAGACTGCTGGGGCAATCAATGGAT GAAAGTGGATTACCACAGCTGACCAGTTATGATTGTGAAGTTAATGCTCCTATACAAGG CAGCAGAAACCTACTGCAGGGTGAAGAATTACTCAGAGCTTTGGATCAAGTTAAC(SEQ ID NO:80; NM_001530), or a nucleic acid sequence that hybridizes to a nucleic acid sequence consisting of SEQ ID NO:80 under stringent hybridization conditions.

In some embodiments, endothelial PAS domain protein 1 (HIF-2α/EPAS1) comprises the amino acid sequence: MTADKEKKRSSSERRKEKSRDAARCRRSKETEVFYELAHELPLPHSVSSHLDKASIMRLAI SFLRTHKLLSSVCSENESEAEADQQMDNLYLKALEGFIAVVTQDGDMIFLSENISKFMGLTQ VELTGHSIFDFTHPCDHEEIRENLSLKNGSGFGKKSKDMSTERDFFMRMKCTVTNRGRTV NLKSATWKVLHCTGQVKVYNNCPPHNSLCGYKEPLLSCLIIMCEPIQHPSHMDIPLDSKTFL SRHSMDMKFTYCDDRITELIGYHPEELLGRSAYEFYHALDSENMTKSHQNLCTKGQVVSG QYRMLAKHGGYVWLETQGTVIYNPRNLQPQCIMCVNYVLSEIEKNDVVFSMDQTESLFKP HLMAMNSIFDSSGKGAVSEKSNFLFTKLKEEPEELAQLAPTPGDAIISLDFGNQNFEESSAY GKAILPPSQPWATELRSHSTQSEAGSLPAFTVPQAAAPGSTTPSATSSSSSCSTPNSPEDY YTSLDNDLKIEVIEKLFAMDTEAKDQCSTQTDFNELDLETLAPYIPMDGEDFQLSPICPEERL LAENPQSTPQHCFSAMTNIFQPLAPVAPHSPFLLDKFQQQLESKKTEPEHRPMSSIFFDAG SKASLPPCCGQASTPLSSMGGRSNTQWPPDPPLHFGPTKWAVGDQRTEFLGAAPLGPPV SPPHVSTFKTRSAKGFGARGPDVLSPAMVALSNKLKLKRQLEYEEQAFQDLSGGDPPGGS TSHLMWKRMKNLRGGSCPLMPDKPLSANVPNDKFTQNPMRGLGHPLRHLPLPQPPSAIS PGENSKSRFPPQCYATQYQDYSLSSAHKVSGMASRLLGPSFESYLLPELTRYDCEVNVPV LGSSTLLQGGDLLRALDQAT (SEQ ID NO:81; NP_001421), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:81.

In some embodiments, the nucleic acid sequence encoding HIF-2a comprises the nucleic acid sequence: ATGACAGCTGACAAGGAGAAGAAAAGGAGTAGCTCGGAGAGGAGGAAGGAGAAGTCC CGGGATGCTGCGCGGTGCCGGCGGAGCAAGGAGACGGAGGTGTTCTATGAGCTGGC CCATGAGCTGCCTCTGCCCCACAGTGTGAGCTCCCATCTGGACAAGGCCTCCATCATG CGACTGGCAATCAGCTTCCTGCGAACACACAAGCTCCTCTCCTCAGTTTGCTCTGAAAA CGAGTCCGAAGCCGAAGCTGACCAGCAGATGGACAACTTGTACCTGAAAGCCTTGGAG GGTTTCATTGCCGTGGTGACCCAAGATGGCGACATGATCTTTCTGTCAGAAAACATCAG CAAGTTCATGGGACTTACACAGGTGGAGCTAACAGGACATAGTATCTTTGACTTCACTC ATCCCTGCGACCATGAGGAGATTCGTGAGAACCTGAGTCTCAAAAATGGCTCTGGTTTT GGGAAAAAAAGCAAAGACATGTCCACAGAGCGGGACTTCTTCATGAGGATGAAGTGCA CGGTCACCAACAGAGGCCGTACTGTCAACCTCAAGTCAGCCACCTGGAAGGTCTTGCA CTGCACGGGCCAGGTGAAAGTCTACAACAACTGCCCTCCTCACAATAGTCTGTGTGGC TACAAGGAGCCCCTGCTGTCCTGCCTCATCATCATGTGTGAACCAATCCAGCACCCAT CCCACATGGACATCCCCCTGGATAGCAAGACCTTCCTGAGCCGCCACAGCATGGACAT GAAGTTCACCTACTGTGATGACAGAATCACAGAACTGATTGGTTACCACCCTGAGGAG CTGCTTGGCCGCTCAGCCTATGAATTCTACCATGCGCTAGACTCCGAGAACATGACCA AGAGTCACCAGAACTTGTGCACCAAGGGTCAGGTAGTAAGTGGCCAGTACCGGATGCT CGCAAAGCATGGGGGCTACGTGTGGCTGGAGACCCAGGGGACGGTCATCTACAACCC TCGCAACCTGCAGCCCCAGTGCATCATGTGTGTCAACTACGTCCTGAGTGAGATTGAG AAGAATGACGTGGTGTTCTCCATGGACCAGACTGAATCCCTGTTCAAGCCCCACCTGA TGGCCATGAACAGCATCTTTGATAGCAGTGGCAAGGGGGCTGTGTCTGAGAAGAGTAA CTTCCTATTCACCAAGCTAAAGGAGGAGCCCGAGGAGCTGGCCCAGCTGGCTCCCAC CCCAGGAGACGCCATCATCTCTCTGGATTTCGGGAATCAGAACTTCGAGGAGTCCTCA GCCTATGGCAAGGCCATCCTGCCCCCGAGCCAGCCATGGGCCACGGAGTTGAGGAGC CACAGCACCCAGAGCGAGGCTGGGAGCCTGCCTGCCTTCACCGTGCCCCAGGCAGCT GCCCCGGGCAGCACCACCCCCAGTGCCACCAGCAGCAGCAGCAGCTGCTCCACGCC CAATAGCCCTGAAGACTATTACACATCTTTGGATAACGACCTGAAGATTGAAGTGATTG AGAAGCTCTTCGCCATGGACACAGAGGCCAAGGACCAATGCAGTACCCAGACGGATTT CAATGAGCTGGACTTGGAGACACTGGCACCCTATATCCCCATGGACGGGGAAGACTTC CAGCTAAGCCCCATCTGCCCCGAGGAGCGGCTCTTGGCGGAGAACCCACAGTCCACC CCCCAGCACTGCTTCAGTGCCATGACAAACATCTTCCAGCCACTGGCCCCTGTAGCCC CGCACAGTCCCTTCCTCCTGGACAAGTTTCAGCAGCAGCTGGAGAGCAAGAAGACAGA GCCCGAGCACCGGCCCATGTCCTCCATCTTCTTTGATGCCGGAAGCAAAGCATCCCTG CCACCGTGCTGTGGCCAGGCCAGCACCCCTCTCTCTTCCATGGGGGGCAGATCCAAT ACCCAGTGGCCCCCAGATCCACCATTACATTTTGGGCCCACAAAGTGGGCCGTCGGG GATCAGCGCACAGAGTTCTTGGGAGCAGCGCCGTTGGGGCCCCCTGTCTCTCCACCC CATGTCTCCACCTTCAAGACAAGGTCTGCAAAGGGTTTTGGGGCTCGAGGCCCAGACG TGCTGAGTCCGGCCATGGTAGCCCTCTCCAACAAGCTGAAGCTGAAGCGACAGCTGG AGTATGAAGAGCAAGCCTTCCAGGACCTGAGCGGGGGGGACCCACCTGGTGGCAGCA CCTCACATTTGATGTGGAAACGGATGAAGAACCTCAGGGGTGGGAGCTGCCCTTTGAT GCCGGACAAGCCACTGAGCGCAAATGTACCCAATGATAAGTTCACCCAAAACCCCATG AGGGGCCTGGGCCATCCCCTGAGACATCTGCCGCTGCCACAGCCTCCATCTGCCATC AGTCCCGGGGAGAACAGCAAGAGCAGGTTCCCCCCACAGTGCTACGCCACCCAGTAC CAGGACTACAGCCTGTCGTCAGCCCACAAGGTGTCAGGCATGGCAAGCCGGCTGCTC GGGCCCTCATTTGAGTCCTACCTGCTGCCCGAACTGACCAGATATGACTGTGAGGTGA ACGTGCCCGTGCTGGGAAGCTCCACGCTCCTGCAAGGAGGGGACCTCCTCAGAGCCC TGGACCAGGCCACC (SEQ ID NO:82; NM_001430), or a nucleic acid sequence that hybridizes to a nucleic acid sequence consisting of SEQ ID NO:82 under stringent hybridization conditions.

In some embodiments, Sonic Headgehog (SHH) comprises the amino acid sequence: MGEMLLLARCLLLVLVSSLLVCSGLACGPGRGFGKRRHPKKLTPLAYKQFIPNVAEKTLGA SGRYEGKISRNSERFKELTPNYNPDIIFKDEENTGADRLMTQRCKDKLNALAISVMNQWPG VKLRVTEGWDEDGHHSEESLHYEGRAVDITTSDRDRSKYGMLARLAVEAGFDWVYYESK AHIHCSVKAENSVAAKSGGCFPGSATVHLEQGGTKLVKDLSPGDRVLAADDQGRLLYSDF LTFLDRDDGAKKVFYVIETREPRERLLLTAAHLLFVAPHNDSATGEPEASSGSGPPSGGAL GPRALFASRVRPGQRVYVVAERDGDRRLLPAAVHSVTLSEEAAGAYAPLTAQGTILINRVL ASCYAVIEEHSWAHRAFAPFRLAHALLAALAPARTDRGGDSGGGDRGGGGGRVALTAPG AADAPGAGATAGIHWYSQLLYQIGTWLLDSEALHPLGMAVKSS(SEQ ID NO:83; NP_000184), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:83.

In some embodiments, the nucleic acid sequence encoding SHH comprises the nucleic acid sequence: ATGGGCGAGATGCTGCTGCTGGCGAGATGTCTGCTGCTAGTCCTCGTCTCCTCGCTGC TGGTATGCTCGGGACTGGCGTGCGGACCGGGCAGGGGGTTCGGGAAGAGGAGGCAC CCCAAAAAGCTGACCCCTTTAGCCTACAAGCAGTTTATCCCCAATGTGGCCGAGAAGA CCCTAGGCGCCAGCGGAAGGTATGAAGGGAAGATCTCCAGAAACTCCGAGCGATTTAA GGAACTCACCCCCAATTACAACCCCGACATCATATTTAAGGATGAAGAAAACACCGGAG CGGACAGGCTGATGACTCAGAGGTGTAAGGACAAGTTGAACGCTTTGGCCATCTCGGT GATGAACCAGTGGCCAGGAGTGAAACTGCGGGTGACCGAGGGCTGGGACGAAGATG GCCACCACTCAGAGGAGTCTCTGCACTACGAGGGCCGCGCAGTGGACATCACCACGT CTGACCGCGACCGCAGCAAGTACGGCATGCTGGCCCGCCTGGCGGTGGAGGCCGGC TTCGACTGGGTGTACTACGAGTCCAAGGCACATATCCACTGCTCGGTGAAAGCAGAGA ACTCGGTGGCGGCCAAATCGGGAGGCTGCTTCCCGGGCTCGGCCACGGTGCACCTG GAGCAGGGCGGCACCAAGCTGGTGAAGGACCTGAGCCCCGGGGACCGCGTGCTGGC GGCGGACGACCAGGGCCGGCTGCTCTACAGCGACTTCCTCACTTTCCTGGACCGCGA CGACGGCGCCAAGAAGGTCTTCTACGTGATCGAGACGCGGGAGCCGCGCGAGCGCC TGCTGCTCACCGCCGCGCACCTGCTCTTTGTGGCGCCGCACAACGACTCGGCCACCG GGGAGCCCGAGGCGTCCTCGGGCTCGGGGCCGCCTTCCGGGGGCGCACTGGGGCC TCGGGCGCTGTTCGCCAGCCGCGTGCGCCCGGGCCAGCGCGTGTACGTGGTGGCCG AGCGTGACGGGGACCGCCGGCTCCTGCCCGCCGCTGTGCACAGCGTGACCCTAAGC GAGGAGGCCGCGGGCGCCTACGCGCCGCTCACGGCCCAGGGCACCATTCTCATCAA CCGGGTGCTGGCCTCGTGCTACGCGGTCATCGAGGAGCACAGCTGGGCGCACCGGG CCTTCGCGCCCTTCCGCCTGGCGCACGCGCTCCTGGCTGCACTGGCGCCCGCGCGC ACGGACCGCGGCGGGGACAGCGGCGGCGGGGACCGCGGGGGCGGCGGCGGCAGA GTAGCCCTAACCGCTCCAGGTGCTGCCGACGCTCCGGGTGCGGGGGCCACCGCGGG CATCCACTGGTACTCGCAGCTGCTCTACCAAATAGGCACCTGGCTCCTGGACAGCGAG GCCCTGCACCCGCTGGGCATGGCGGTCAAGTCCAGC (SEQ ID NO:84; NM_000193), or a nucleic acid sequence that hybridizes to a nucleic acid sequence consisting of SEQ ID NO:84 under stringent hybridization conditions.

In some embodiments, desert hedgehog (DHH) comprises the amino acid sequence: MALLTNLLPLCCLALLALPAQSCGPGRGPVGRRRYARKQLVPLLYKQFVPGVPERTLGASG PAEGRVARGSERFRDLVPNYNPDIIFKDEENSGADRLMTERCKERVNALAIAVMNMWPGV RLRVTEGWDEDGHHAQDSLHYEGRALDITTSDRDRNKYGLLARLAVEAGFDWVYYESRN HVHVSVKADNSLAVRAGGCFPGNATVRLWSGERKGLRELHRGDWVLAADASGRVVPTPV LLFLDRDLQRRASFVAVETEWPPRKLLLTPWHLVFAARGPAPAPGDFAPVFARRLRAGDS VLAPGGDALRPARVARVAREEAVGVFAPLTAHGTLLVNDVLASCYAVLESHQWAHRAFAP LRLLHALGALLPGGAVQPTGMHWYSRLLYRLAEELLG (SEQ ID NO:85; NP_066382), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:85.

In some embodiments, the nucleic acid sequence encoding DHH comprises the nucleic acid sequence: ATGGCTCTCCTGACCAATCTACTGCCCCTGTGCTGCTTGGCACTTCTGGCGCTGCCAG CCCAGAGCTGCGGGCCGGGCCGGGGGCCGGTTGGCCGGCGCCGCTATGCGCGCAA GCAGCTCGTGCCGCTACTCTACAAGCAATTTGTGCCCGGCGTGCCAGAGCGGACCCT GGGCGCCAGTGGGCCAGCGGAGGGGAGGGTGGCAAGGGGCTCCGAGCGCTTCCGG GACCTCGTGCCCAACTACAACCCCGACATCATCTTCAAGGATGAGGAGAACAGTGGAG CCGACCGCCTGATGACCGAGCGTTGTAAGGAGCGGGTGAACGCTTTGGCCATTGCCG TGATGAACATGTGGCCCGGAGTGCGCCTACGAGTGACTGAGGGCTGGGACGAGGACG GCCACCACGCTCAGGATTCACTCCACTACGAAGGCCGTGCTTTGGACATCACTACGTC TGACCGCGACCGCAACAAGTATGGGTTGCTGGCGCGCCTCGCAGTGGAAGCCGGCTT CGACTGGGTCTACTACGAGTCCCGCAACCACGTCCACGTGTCGGTCAAAGCTGATAAC TCACTGGCGGTCCGGGCGGGCGGCTGCTTTCCGGGAAATGCAACTGTGCGCCTGTGG AGCGGCGAGCGGAAAGGGCTGCGGGAACTGCACCGCGGAGACTGGGTTTTGGCGGC CGATGCGTCAGGCCGGGTGGTGCCCACGCCGGTGCTGCTCTTCCTGGACCGGGACTT GCAGCGCCGGGCTTCATTTGTGGCTGTGGAGACCGAGTGGCCTCCACGCAAACTGTT GCTCACGCCCTGGCACCTGGTGTTTGCCGCTCGAGGGCCGGCGCCCGCGCCAGGCG ACTTTGCACCGGTGTTCGCGCGCCGGCTACGCGCTGGGGACTCGGTGCTGGCGCCC GGCGGGGATGCGCTTCGGCCAGCGCGCGTGGCCCGTGTGGCGCGGGAGGAAGCCG TGGGCGTGTTCGCGCCGCTCACCGCGCACGGGACGCTGCTGGTGAACGATGTCCTGG CCTCTTGCTACGCGGTTCTGGAGAGTCACCAGTGGGCGCACCGCGCTTTTGCCCCCTT GAGACTGCTGCACGCGCTAGGGGCGCTGCTCCCCGGCGGGGCCGTCCAGCCGACTG GCATGCATTGGTACTCTCGGCTCCTCTACCGCTTAGCGGAGGAGCTACTGGGC (SEQ ID NO:86; NM_021044), or a nucleic acid sequence that hybridizes to a nucleic acid sequence consisting of SEQ ID NO:86 under stringent hybridization conditions.

In some embodiments, Indian Hedgehog (IHH) comprises the amino acid sequence: MSPARLRPRLHFCLVLLLLLVVPAAWGCGPGRVVGSRRRPPRKLVPLAYKQFSPNVPEKT LGASGRYEGKIARSSERFKELTPNYNPDllFKDEENTGADRLMTQRCKDRLNSLAISVMNQ WPGVKLRVTEGWDEDGHHSEESLHYEGRAVDITTSDRDRNKYGLLARLAVEAGFDWVYY ESKAHVHCSVKSEHSAAAKTGGCFPAGAQVRLESGARVALSAVRPGDRVLAMGEDGSPT FSDVLIFLDREPHRLRAFQVIETQDPPRRLALTPAHLLFTADNHTEPAARFRATFASHVQPG QYVLVAGVPGLQPARVAAVSTHVALGAYAPLTKHGTLVVEDVVASCFAAVADHHLAQLAF WPLRLFHSLAWGSWTPGEGVHWYPQLLYRLGRLLLEEGSFHPLGMSGAGS (SEQ ID NO:87; NP_002172), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:87.

In some embodiments, the nucleic acid sequence encoding IHH comprises the nucleic acid sequence: ATGTCTCCCGCCCGGCTCCGGCCCCGACTGCACTTCTGCCTGGTCCTGTTGCTGCTGC TGGTGGTGCCGGCGGCATGGGGCTGCGGGCCGGGTCGGGTGGTGGGCAGCCGCCG GCGACCGCCACGCAAACTCGTGCCGCTCGCCTACAAGCAGTTCAGCCCCAATGTGCC CGAGAAGACCCTGGGCGCCAGCGGACGCTATGAAGGCAAGATCGCTCGCAGCTCCGA GCGCTTCAAGGAGCTCACCCCCAATTACAATCCAGACATCATCTTCAAGGACGAGGAG AACACAGGCGCCGACCGCCTCATGACCCAGCGCTGCAAGGACCGCCTGAACTCGCTG GCTATCTCGGTGATGAACCAGTGGCCCGGTGTGAAGCTGCGGGTGACCGAGGGCTGG GACGAGGACGGCCACCACTCAGAGGAGTCCCTGCATTATGAGGGCCGCGCGGTGGAC ATCACCACATCAGACCGCGACCGCAATAAGTATGGACTGCTGGCGCGCTTGGCAGTG GAGGCCGGCTTTGACTGGGTGTATTACGAGTCAAAGGCCCACGTGCATTGCTCCGTCA AGTCCGAGCACTCGGCCGCAGCCAAGACAGGCGGCTGCTTCCCTGCCGGAGCCCAG GTACGCCTGGAGAGTGGGGCGCGTGTGGCCTTGTCAGCCGTGAGGCCGGGAGACCG TGTGCTGGCCATGGGGGAGGATGGGAGCCCCACCTTCAGCGATGTGCTCATTTTCCTG GACCGCGAGCCTCACAGGCTGAGAGCCTTCCAGGTCATCGAGACTCAGGACCCCCCA CGCCGCCTGGCACTCACACCCGCTCACCTGCTCTTTACGGCTGACAATCACACGGAGC CGGCAGCCCGCTTCCGGGCCACATTTGCCAGCCACGTGCAGCCTGGCCAGTACGTGC TGGTGGCTGGGGTGCCAGGCCTGCAGCCTGCCCGCGTGGCAGCTGTCTCTACACACG TGGCCCTCGGGGCCTACGCCCCGCTCACAAAGCATGGGACACTGGTGGTGGAGGATG TGGTGGCATCCTGCTTCGCGGCCGTGGCTGACCACCACCTGGCTCAGTTGGCCTTCT GGCCCCTGAGACTCTTTCACAGCTTGGCATGGGGCAGCTGGACCCCGGGGGAGGGTG TGCATTGGTACCCCCAGCTGCTCTACCGCCTGGGGCGTCTCCTGCTAGAAGAGGGCA GCTTCCACCCACTGGGCATGTCCGGGGCAGGGAGC (SEQ ID NO:88; NM_002181), or a nucleic acid sequence that hybridizes to a nucleic acid sequence consisting of SEQ ID NO:88 under stringent hybridization conditions.

In some embodiments, Brachyury (TBXT) comprises the amino acid sequence: MSSPGTESAGKSLQYRVDHLLSAVENELQAGSEKGDPTERELRVGLEESELWLRFKELTN EMIVTKNGRRMFPVLKVNVSGLDPNAMYSFLLDFVAADNHRWKYVNGEWVPGGKPEPQA PSCVYIHPDSPNFGAHWMKAPVSFSKVKLTNKLNGGGQIMLNSLHKYEPRIHIVRVGGPQR MITSHCFPETQFIAVTAYQNEEITALKIKYNPFAKAFLDAKERSDHKEMMEEPGDSQQPGYS QSGGWLLPGTSTLCPPANPHPQFGGALSLPSTHSCDRYPTLRSHRSSPYPSPYAHRNNSP TYSDNSPACLSMLQSHDNWSSLGMPAHPSMLPVSHNASPPTSSSQYPSLWSVSNGAVTP GSQAAAVSNGLGAQFFRGSPAHYTPLTHPVSAPSSSGSPLYEGAAAATDIVDSQYDAAAQ GRLIASWTPVSPPSM (SEQ ID NO:89; NP_003172), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:89.

In some embodiments, the nucleic acid sequence encoding TBXT comprises the nucleic acid sequence: ATGAGCTCCCCTGGCACCGAGAGCGCGGGAAAGAGCCTGCAGTACCGAGTGGACCAC CTGCTGAGCGCCGTGGAGAATGAGCTGCAGGCGGGCAGCGAGAAGGGCGACCCCAC AGAGCGCGAACTGCGCGTGGGCCTGGAGGAGAGCGAGCTGTGGCTGCGCTTCAAGG AGCTCACCAATGAGATGATCGTGACCAAGAACGGCAGGAGGATGTTTCCGGTGCTGAA GGTGAACGTGTCTGGCCTGGACCCCAACGCCATGTACTCCTTCCTGCTGGACTTCGTG GCGGCGGACAACCACCGCTGGAAGTACGTGAACGGGGAATGGGTGCCGGGGGGCAA GCCGGAGCCGCAGGCGCCCAGCTGCGTCTACATCCACCCCGACTCGCCCAACTTCGG GGCCCACTGGATGAAGGCTCCCGTCTCCTTCAGCAAAGTCAAGCTCACCAACAAGCTC AACGGAGGGGGCCAGATCATGCTGAACTCCTTGCATAAGTATGAGCCTCGAATCCACA TAGTGAGAGTTGGGGGTCCACAGCGCATGATCACCAGCCACTGCTTCCCTGAGACCCA GTTCATAGCGGTGACTGCTTATCAGAACGAGGAGATCACAGCTCTTAAAATTAAGTACA ATCCATTTGCAAAAGCTTTCCTTGATGCAAAGGAAAGAAGTGATCACAAAGAGATGATG GAGGAACCCGGAGACAGCCAGCAACCTGGGTACTCCCAATCAGGGGGGTGGCTTCTT CCTGGAACCAGCACCCTGTGTCCACCTGCAAATCCTCATCCTCAGTTTGGAGGTGCCC TCTCCCTCCCCTCCACGCACAGCTGTGACAGGTACCCAACCCTGAGGAGCCACCGGT CCTCACCCTACCCCAGCCCCTATGCTCATCGGAACAATTCTCCAACCTATTCTGACAAC TCACCTGCATGTTTATCCATGCTGCAATCCCATGACAATTGGTCCAGCCTTGGAATGCC TGCCCATCCCAGCATGCTCCCCGTGAGCCACAATGCCAGCCCACCTACCAGCTCCAGT CAGTACCCCAGCCTGTGGTCTGTGAGCAACGGCGCCGTCACCCCGGGCTCCCAGGCA GCAGCCGTGTCCAACGGGCTGGGGGCCCAGTTCTTCCGGGGCTCCCCCGCGCACTAC ACACCCCTCACCCATCCGGTCTCGGCGCCCTCTTCCTCGGGATCCCCACTGTACGAAG GGGCGGCCGCGGCCACAGACATCGTGGACAGCCAGTACGACGCCGCAGCCCAAGGC CGCCTCATAGCCTCATGGACACCTGTGTCGCCACCTTCCATG (SEQ ID NO:90; NM_003181), or a nucleic acid sequence that hybridizes to a nucleic acid sequence consisting of SEQ ID NO:90 under stringent hybridization conditions.

In some embodiments, T-box brain 1 (TBR1) comprises the amino acid sequence: MQLEHCLSPSIMLSKKFLNVSSSYPHSGGSELVLHDHPIlSTTDNLERSSPLKKITRGMTNQ SDTDNFPDSKDSPGDVQRSKLSPVLDGVSELRHSFDGSAADRYLLSQSSQPQSAATAPSA MFPYPGQHGPAHPAFSIGSPSRYMAHHPVITNGAYNSLLSNSSPQGYPTAGYPYPQQYGH SYQGAPFYQFSSTQPGLVPGKAQVYLCNRPLWLKFHRHQTEMIITKQGRRMFPFLSFNISG LDPTAHYNIFVDVILADPNHWRFQGGKWVPCGKADTNVQGNRVYMHPDSPNTGAHWMR QEISFGKLKLTNNKGASNNNGQMVVLQSLHKYQPRLHVVEVNEDGTEDTSQPGRVQTFTF PETQFIAVTAYQNTDITQLKIDHNPFAKGFRDNYDTIYTGCDMDRLTPSPNDSPRSQIVPGA RYAMAGSFLQDQFVSNYAKARFHPGAGAGPGPGTDRSVPHTNGLLSPQQAEDPGAPSPQ RWFVTPANNRLDFAASAYDTATDFAGNAATLLSYAAAGVKALPLQAAGCTGRPLGYYADP SGWGARSPPQYCGTKSGSVLPCWPNSAAAAARMAGANPYLGEEAEGLAAERSPLPPGA AEDAKPKDLSDSSWIETPSSIKSIDSSDSGIYEQAKRRRISPADTPVSESSSPLKSEVLAQRD CEKNCAKDISGYYGFYSHS (SEQ ID NO:91; NP_006584), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:91.

In some embodiments, the nucleic acid sequence encoding TBR1 comprises the nucleic acid sequence: ATGCAGCTGGAGCACTGCCTTTCTCCTTCTATCATGCTCTCCAAGAAATTTCTCAATGT GAGCAGCAGCTACCCACATTCAGGCGGATCCGAGCTTGTCTTGCACGATCATCCCATT ATCTCGACCACTGACAACCTGGAGAGAAGTTCACCTTTGAAAAAAATTACCAGGGGGAT GACGAATCAGTCAGATACAGACAATTTTCCTGACTCCAAGGACTCACCAGGGGACGTC CAGAGAAGTAAACTCTCTCCTGTCTTGGACGGGGTCTCTGAGCTTCGTCACAGTTTCGA TGGCTCTGCTGCAGATCGCTACCTCCTCTCTCAGTCCAGCCAGCCACAGTCTGCGGCC ACTGCTCCCAGTGCCATGTTCCCGTACCCCGGCCAGCACGGACCGGCGCACCCCGCC TTCTCCATCGGCAGCCCTAGCCGCTACATGGCCCACCACCCGGTCATCACCAACGGAG CCTACAACAGCCTCCTGTCCAACTCCTCGCCGCAGGGATACCCCACGGCCGGCTACC CCTACCCACAGCAGTACGGCCACTCCTACCAAGGAGCTCCGTTCTACCAGTTCTCCTC CACCCAGCCGGGGCTGGTGCCCGGCAAAGCACAGGTGTACCTGTGCAACAGGCCCCT TTGGCTGAAATTTCACCGGCACCAAACGGAGATGATCATCACCAAACAGGGAAGGCGC ATGTTTCCTTTTTTAAGTTTTAACATTTCTGGTCTCGATCCCACGGCTCATTACAATATTT TTGTGGATGTGATTTTGGCGGATCCCAATCACTGGAGGTTTCAAGGAGGCAAATGGGT TCCTTGCGGCAAAGCGGACACCAATGTGCAAGGAAATCGGGTCTATATGCATCCGGAT TCCCCCAACACTGGGGCTCACTGGATGCGCCAAGAAATCTCTTTTGGAAAATTAAAACT TACGAACAACAAAGGAGCTTCAAATAACAATGGGCAGATGGTGGTTTTACAGTCCTTGC ACAAGTACCAGCCCCGCCTGCATGTGGTGGAAGTGAACGAGGACGGCACGGAGGACA CTAGCCAGCCCGGCCGCGTGCAGACGTTCACTTTCCCTGAGACTCAGTTCATCGCCGT CACCGCCTACCAGAACACGGATATTACACAACTGAAAATAGATCACAACCCTTTTGCAA AAGGATTTCGGGATAATTATGACACGATCTACACCGGCTGTGACATGGACCGCCTGAC CCCCTCGCCCAACGACTCGCCGCGCTCGCAGATCGTGCCCGGGGCCCGCTACGCCAT GGCCGGCTCTTTCCTGCAGGACCAGTTCGTGAGCAACTACGCCAAGGCCCGCTTCCA CCCGGGCGCGGGCGCGGGCCCCGGGCCGGGTACGGACCGCAGCGTGCCGCACACC AACGGGCTGCTGTCGCCGCAGCAGGCCGAGGACCCGGGCGCGCCCTCGCCGCAACG CTGGTTTGTGACGCCGGCCAACAACCGGCTGGACTTCGCGGCCTCGGCCTATGACAC GGCCACGGACTTCGCGGGCAACGCGGCCACGCTGCTCTCTTACGCGGCGGCGGGCG TGAAGGCGCTGCCGCTGCAGGCTGCAGGCTGCACTGGCCGCCCGCTCGGCTACTACG CCGACCCGTCGGGCTGGGGCGCCCGCAGTCCCCCGCAGTACTGCGGCACCAAGTCG GGCTCGGTGCTGCCCTGCTGGCCCAACAGCGCCGCGGCCGCCGCGCGCATGGCCGG CGCCAATCCCTACCTGGGCGAGGAGGCCGAGGGCCTGGCCGCCGAGCGCTCGCCGC TGCCGCCCGGCGCCGCCGAGGACGCCAAGCCCAAGGACCTGTCCGATTCCAGCTGG ATCGAGACGCCCTCCTCGATCAAGTCCATCGACTCCAGCGACTCGGGGATTTACGAGC AGGCCAAGCGGAGGCGGATCTCGCCGGCCGACACGCCCGTGTCCGAGAGTTCGTCC CCGCTCAAGAGCGAGGTGCTGGCCCAGCGGGACTGCGAGAAGAACTGCGCCAAGGA CATTAGCGGCTACTATGGCTTCTACTCGCACAGC (SEQ ID NO:92; NM_006593), or a nucleic acid sequence that hybridizes to a nucleic acid sequence consisting of SEQ ID NO:92 under stringent hybridization conditions.

In some embodiments, T-Box 1 (TBX1) comprises the amino acid sequence: MHFSTVTRDMEAFTASSLSSLGAAGGFPGAASPGADPYGPREPPPPPRYDPCAAAAPGA PGPPPPPHAYPFAPAAGAATSAAAEPEGPGASCAAAAKAPVKKNAKVAGVSVQLEMKAL WDEFNQLGTEMIVTKAGRRMFPTFQVKLFGMDPMADYMLLMDFVPVDDKRYRYAFHSSS WLVAGKADPATPGRVHYHPDSPAKGAQWMKQIVSFDKLKLTNNLLDDNGHIILNSMHRYQ PRFHVVYVDPRKDSEKYAEENFKTFVFEETRFTAVTAYQNHRITQLKIASNPFAKGFRDCD PEDWPRNHRPGALPLMSAFARSRNPVASPTQPSGTEKDAAEARREFQRDAGGPAVLGDP AHPPQLLARVLSPSLPGAGGAGGLVPLPGAPGGRPSPPNPELRLEAPGASEPLHHHPYKY PAAAYDHYLGAKSRPAPYPLPGLRGHGYHPHAHPHHHHHPVSPAAAAAAAAAAAAAAAN MYSSAGAAPPGSYDYCPR (SEQ ID NO:93; NP_542377), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:93.

In some embodiments, the nucleic acid sequence encoding TBX1 comprises the nucleic acid sequence: ATGCACTTCAGCACCGTCACCAGGGACATGGAAGCCTTCACGGCCAGCAGCCTGAGC AGCCTGGGGGCCGCGGGGGGCTTCCCGGGCGCCGCGTCGCCCGGCGCCGACCCGT ACGGCCCGCGCGAGCCCCCGCCGCCGCCGCGCTACGACCCGTGCGCCGCCGCCGC CCCCGGCGCCCCGGGTCCGCCGCCGCCGCCGCACGCCTACCCGTTTGCGCCGGCCG CCGGGGCCGCCACCAGCGCCGCCGCCGAGCCCGAGGGCCCCGGGGCCAGCTGCGC GGCCGCAGCCAAGGCGCCGGTGAAGAAGAACGCGAAGGTGGCCGGTGTGAGCGTGC AGCTAGAGATGAAGGCGCTGTGGGACGAGTTCAACCAGCTGGGCACCGAGATGATCG TCACCAAGGCCGGCAGGCGGATGTTTCCCACCTTCCAAGTGAAGCTCTTCGGCATGGA TCCCATGGCCGACTATATGCTGCTCATGGACTTCGTGCCGGTGGACGATAAGCGCTAC CGGTACGCCTTCCACAGCTCCTCCTGGCTGGTGGCGGGGAAGGCCGACCCTGCCACG CCAGGCCGCGTGCACTACCACCCGGACTCGCCTGCCAAGGGCGCGCAGTGGATGAA GCAAATCGTGTCCTTCGACAAGCTCAAGCTGACCAACAACTTACTGGACGACAACGGC CACATTATTCTGAATTCCATGCACAGATACCAGCCCCGCTTCCACGTGGTCTATGTGGA CCCACGCAAAGATAGCGAGAAATATGCCGAGGAGAACTTCAAAACCTTTGTGTTCGAG GAGACACGATTCACCGCGGTCACTGCCTACCAGAACCATCGGATCACGCAGCTCAAGA TTGCCAGCAATCCCTTCGCGAAAGGCTTCCGGGACTGTGACCCTGAGGACTGGCCCC GGAACCACCGGCCCGGCGCACTGCCGCTCATGAGCGCCTTCGCGCGCTCGCGGAAC CCCGTGGCTTCCCCGACGCAGCCCAGCGGCACGGAGAAAGACGCGGCTGAGGCCCG GCGAGAATTCCAGCGCGACGCGGGCGGGCCAGCGGTGCTCGGGGACCCGGCGCATC CTCCGCAGCTGCTGGCCCGGGTGCTAAGCCCCTCGCTGCCCGGGGCCGGCGGCGCC GGCGGCTTAGTCCCGCTGCCCGGCGCGCCCGGAGGCCGGCCCAGTCCCCCGAACCC CGAGCTGCGCCTGGAGGCGCCCGGCGCATCGGAGCCGCTGCACCACCACCCCTACA AATATCCGGCCGCCGCCTACGACCACTATCTCGGGGCCAAGAGCCGGCCGGCGCCCT ACCCGCTGCCCGGCCTGCGTGGCCACGGCTACCACCCGCACGCGCATCCGCACCAC CACCACCACCCCGTGAGTCCAGCCGCCGCGGCCGCCGCCGCCGCTGCCGCAGCTGC CGCGGCCGCCAACATGTACTCGTCGGCCGGAGCCGCGCCGCCCGGCTCCTACGACTA TTGCCCCAGA (SEQ ID NO:94; NM_080647), or a nucleic acid sequence that hybridizes to a nucleic acid sequence consisting of SEQ ID NO:94 under stringent hybridization conditions.

In some embodiments, T-box 2 (TBX2) comprises the amino acid sequence: MREPALAASAMAYHPFHAPRPADFPMSAFLAAAQPSFFPALALPPGALAKPLPDPGLAGAA AAAAAAAAAAEAGLHVSALGPHPPAAHLRSLKSLEPEDEVEDDPKVTLEAKELWDQFHKLG TEMVITKSGRRMFPPFKVRVSGLDKKAKYILLMDIVAADDCRYKFHNSRWMVAGKADPEM PKRMYIHPDSPATGEQWMAKPVAFHKLKLTNNISDKHGFTILNSMHKYQPRFHIVRANDILK LPYSTFRTYVFPETDFIAVTAYQNDKITQLKIDNNPFAKGFRDTGNGRREKRKQLTLPSLRL YEEHCKPERDGAESDASSCDPPPAREPPTSPGAAPSPLRLHRARAEEKSCAADSDPEPER LSEERAGAPLGRSPAPDSASPTRLTEPERARERRSPERGKEPAESGGDGPFGLRSLEKER AEARRKDEGRKEAAEGKEQGLAPLVVQTDSASPLGAGHLPGLAFSSHLHGQQFFGPLGA GQPLFLHPGQFTMGPGAFSAMGMGHLLASVAGGGNGGGGGPGTAAGLDAGGLGPAASA ASTAAPFPFHLSQHMLASQGIPMPTFGGLFPYPYTYMAAAAAAASALPATSAAAAAAAAAG SLSRSPFLGSARPRLRFSPYQIPVTIPPSTSLLTTGLASEGSKAAGGNSREPSPLPELALRK VGAPSRGALSPSGSAKEAANELQSIQRLVSGLESQRALSPGRESPK (SEQ ID NO:95; NP_005985), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:95.

In some embodiments, the nucleic acid sequence encoding TBX2 comprises the nucleic acid sequence: ATGAGAGAGCCGGCGCTGGCGGCCAGCGCCATGGCTTACCACCCGTTCCACGCGCCA CGGCCCGCCGACTTCCCCATGTCCGCCTTTCTGGCGGCGGCGCAGCCCTCCTTCTTC CCGGCACTCGCGCTGCCGCCCGGCGCGCTGGCCAAGCCGCTGCCCGACCCGGGCCT GGCGGGGGCGGCGGCCGCGGCGGCGGCGGCGGCAGCAGCGGCCGAGGCGGGGCT GCACGTCTCGGCACTGGGCCCGCACCCGCCCGCCGCGCATCTGCGCTCCCTCAAGAG CCTGGAGCCCGAGGACGAGGTGGAGGACGACCCCAAGGTGACGCTGGAGGCCAAGG AGCTGTGGGACCAGTTCCACAAGCTAGGCACGGAGATGGTCATCACCAAGTCCGGGA GGCGGATGTTCCCCCCCTTCAAGGTGCGAGTCAGCGGCCTGGACAAGAAGGCCAAGT ATATCCTGCTGATGGACATTGTAGCCGCTGACGATTGCCGCTATAAGTTCCACAACTCG CGCTGGATGGTGGCGGGCAAGGCCGACCCTGAGATGCCCAAACGCATGTACATCCAC CCAGACAGCCCAGCCACGGGGGAGCAGTGGATGGCTAAGCCTGTGGCCTTCCACAAG CTGAAGCTGACCAACAACATCTCTGACAAGCACGGCTTCACCATCCTAAACTCCATGCA CAAGTACCAGCCGCGCTTCCACATAGTGCGAGCCAACGACATCCTGAAGCTGCCTTAC AGCACCTTCCGCACCTACGTGTTCCCGGAGACCGACTTCATCGCCGTCACTGCCTACC AGAATGACAAGATCACACAGCTGAAGATCGACAACAACCCGTTTGCCAAGGGCTTCCG GGACACCGGGAACGGCCGGCGGGAGAAAAGGAAGCAGCTGACGCTGCCGTCTCTAC GCTTGTACGAGGAGCACTGCAAACCCGAGCGCGATGGCGCGGAGTCAGACGCCTCGT CGTGCGACCCTCCCCCCGCGCGGGAACCACCCACCTCCCCGGGCGCAGCGCCCAGT CCGCTGCGCCTGCACCGGGCCCGAGCTGAGGAGAAGTCGTGCGCCGCGGACAGCGA CCCGGAGCCTGAGCGGTTGAGCGAGGAGCGTGCGGGGGCGCCGCTAGGCCGCAGC CCGGCTCCAGACAGCGCCAGCCCCACTCGCTTGACCGAACCCGAGCGCGCCCGGGA GCGGCGTAGTCCCGAGAGGGGCAAGGAGCCGGCCGAGAGCGGCGGGGACGGCCCG TTCGGCCTGAGGAGCCTGGAGAAGGAGCGCGCCGAAGCTCGGAGGAAGGACGAGGG GCGCAAGGAGGCGGCCGAGGGCAAGGAGCAGGGCCTGGCGCCGCTGGTGGTGCAG ACAGACAGTGCGTCCCCCCTGGGCGCCGGACACCTGCCCGGCCTGGCCTTTTCCAGC CACTTGCACGGGCAGCAGTTCTTTGGGCCGCTGGGAGCCGGCCAGCCGCTCTTCCTG CACCCTGGACAGTTCACCATGGGCCCTGGCGCCTTCTCCGCCATGGGCATGGGTCAC CTACTGGCCTCGGTGGCAGGCGGCGGCAACGGCGGAGGTGGCGGGCCTGGGACCG CCGCGGGGCTGGACGCAGGCGGGCTGGGTCCCGCGGCCAGCGCAGCAAGCACCGC CGCGCCCTTCCCGTTCCACCTCTCCCAGCACATGCTGGCATCTCAGGGAATTCCAATG CCCACTTTCGGAGGCCTCTTCCCCTACCCCTACACCTACATGGCAGCAGCAGCCGCAG CCGCCTCGGCTTTGCCCGCCACTAGTGCTGCAGCTGCCGCCGCCGCAGCCGCCGGCT CCCTCTCCCGGAGCCCCTTCCTGGGCAGTGCCCGGCCCCGACTGCGTTTCAGCCCCT ATCAGATCCCGGTCACCATCCCGCCTAGCACTAGCCTCCTCACCACCGGGCTGGCCTC TGAGGGCTCCAAGGCCGCTGGTGGAAACAGCCGGGAGCCTAGCCCCCTGCCCGAGC TGGCTCTCCGCAAAGTAGGGGCCCCATCCCGCGGTGCCCTGTCGCCCAGTGGCTCGG CCAAGGAGGCGGCCAATGAACTGCAGAGCATCCAGAGACTGGTGAGTGGGCTGGAGA GCCAGCGAGCCCTCTCCCCAGGCCGGGAGTCGCCCAAG (SEQ ID NO:96; NM_005994), or a nucleic acid sequence that hybridizes to a nucleic acid sequence consisting of SEQ ID NO:96 under stringent hybridization conditions.

In some embodiments, T-box 3 (TBX3) comprises the amino acid sequence: MSLSMRDPVIPGTSMAYHPFLPHRAPDFAMSAVLGHQPPFFPALTLPPNGAAALSLPGALA KPIMDQLVGAAETGIPFSSLGPQAHLRPLKTMEPEEEVEDDPKVHLEAKELWDQFHKRGTE MVITKSGRRMFPPFKVRCSGLDKKAKYILLMDIIAADDCRYKFHNSRWMVAGKADPEMPKR MYIHPDSPATGEQWMSKVVTFH KLKLTNNISDKHGFTLAFPSDHATWQGNYSFGTQTILNS MHKYQPRFHIVRANDILKLPYSTFRTYLFPETEFIAVTAYQNDKITQLKIDNNPFAKGFRDTG NGRREKRKQLTLQSMRVFDERHKKENGTSDESSSEQAAFNCFAQASSPAASTVGTSNLK DLCPSEGESDAEAESKEEHGPEACDAAKISTTTSEEPCRDKGSPAVKAHLFAAERPRDSG RLDKASPDSRHSPATISSSTRGLGAEERRSPVREGTAPAKVEEARALPGKEAFAPLTVQTD AAAAHLAQGPLPGLGFAPGLAGQQFFNGHPLFLHPSQFAMGGAFSSMAAAGMGPLLATV SGASTGVSGLDSTAMASAAAAQGLSGASAATLPFHLQQHVLASQGLAMSPFGSLFPYPYT YMAAAAAASSAAASSSVHRHPFLNLNTMRPRLRYSPYSIPVPVPDGSSLLTTALPSMAAAA GPLDGKVAALAASPASVAVDSGSELNSRSSTLSSSSMSLSPKLCAEKEAATSELQSIQRLV SGLEAKPDRSRSASP (SEQ ID NO:97; NP_057653), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:97.

In some embodiments, the nucleic acid sequence encoding TBX3 comprises the nucleic acid sequence ATGAGCCTCTCCATGAGAGATCCGGTCATTCCTGGGACAAGCATGGCCTACCATCCGT TCCTACCTCACCGGGCGCCGGACTTCGCCATGAGCGCGGTGCTGGGTCACCAGCCGC CGTTCTTCCCCGCGCTGACGCTGCCTCCCAACGGCGCGGCGGCGCTCTCGCTGCCGG GCGCCCTGGCCAAGCCGATCATGGATCAATTGGTGGGGGCGGCCGAGACCGGCATCC CGTTCTCCTCCCTGGGGCCCCAGGCGCATCTGAGGCCTTTGAAGACCATGGAGCCCG AAGAAGAGGTGGAGGACGACCCCAAGGTGCACCTGGAGGCTAAAGAACTTTGGGATC AGTTTCACAAGCGGGGCACCGAGATGGTCATTACCAAGTCGGGAAGGCGAATGTTTCC TCCATTTAAAGTGAGATGTTCTGGGCTGGATAAAAAAGCCAAATACATTTTATTGATGGA CATTATAGCTGCTGATGACTGTCGTTATAAATTTCACAATTCTCGGTGGATGGTGGCTG GTAAGGCCGACCCCGAAATGCCAAAGAGGATGTACATTCACCCGGACAGCCCCGCTA CTGGGGAACAGTGGATGTCCAAAGTCGTCACTTTCCACAAACTGAAACTCACCAACAAC ATTTCAGACAAACATGGATTTACTTTGGCCTTCCCAAGTGATCACGCTACGTGGCAGGG GAATTATAGTTTTGGTACTCAGACTATATTGAACTCCATGCACAAATACCAGCCCCGGTT CCACATTGTAAGAGCCAATGACATCTTGAAACTCCCTTATAGTACATTTCGGACATACTT GTTCCCCGAAACTGAATTCATCGCTGTGACTGCATACCAGAATGATAAGATAACCCAGT TAAAAATAGACAACAACCCTTTTGCAAAAGGTTTCCGGGACACTGGAAATGGCCGAAGA GAAAAAAGAAAACAGCTCACCCTGCAGTCCATGAGGGTGTTTGATGAAAGACACAAAAA GGAGAATGGGACCTCTGATGAGTCCTCCAGTGAACAAGCAGCTTTCAACTGCTTCGCC CAGGCTTCTTCTCCAGCCGCCTCCACTGTAGGGACATCGAACCTCAAAGATTTATGTCC CAGCGAGGGTGAGAGCGACGCCGAGGCCGAGAGCAAAGAGGAGCATGGCCCCGAGG CCTGCGACGCGGCCAAGATCTCCACCACCACGTCGGAGGAGCCCTGCCGTGACAAGG GCAGCCCCGCGGTCAAGGCTCACCTTTTCGCTGCTGAGCGGCCCCGGGACAGCGGG CGGCTGGACAAAGCGTCGCCCGACTCACGCCATAGCCCCGCCACCATCTCGTCCAGC ACTCGCGGCCTGGGCGCGGAGGAGCGCAGGAGCCCGGTTCGCGAGGGCACAGCGC CGGCCAAGGTGGAAGAGGCGCGCGCGCTCCCGGGCAAGGAGGCCTTCGCGCCGCTC ACGGTGCAGACGGACGCGGCCGCCGCGCACCTGGCCCAGGGCCCCCTGCCTGGCCT CGGCTTCGCCCCGGGCCTGGCGGGCCAACAGTTCTTCAACGGGCACCCGCTCTTCCT GCACCCCAGCCAGTTTGCCATGGGGGGCGCCTTCTCCAGCATGGCGGCCGCTGGCAT GGGTCCCCTCCTGGCCACGGTTTCTGGGGCCTCCACCGGTGTCTCGGGCCTGGATTC CACGGCCATGGCCTCTGCCGCTGCGGCGCAGGGACTGTCCGGGGCGTCCGCGGCCA CCCTGCCCTTCCACCTCCAGCAGCACGTCCTGGCCTCTCAGGGCCTGGCCATGTCCC CTTTCGGAAGCCTGTTCCCTTACCCCTACACGTACATGGCCGCAGCGGCGGCCGCCTC CTCTGCGGCAGCCTCCAGCTCGGTGCACCGCCACCCCTTCCTCAATCTGAACACCATG CGCCCGCGGCTGCGCTACAGCCCCTACTCCATCCCGGTGCCGGTCCCGGACGGCAG CAGTCTGCTCACCACCGCCCTGCCCTCCATGGCGGCGGCCGCGGGGCCCCTGGACG GCAAAGTCGCCGCCCTGGCCGCCAGCCCGGCCTCGGTGGCAGTGGACTCGGGCTCT GAACTCAACAGCCGCTCCTCCACGCTCTCCTCCAGCTCCATGTCCTTGTCGCCCAAAC TCTGCGCGGAGAAAGAGGCGGCCACCAGCGAACTGCAGAGCATCCAGCGGTTGGTTA GCGGCTTGGAAGCCAAGCCGGACAGGTCCCGCAGCGCGTCCCCG (SEQ ID NO:98; NM_016569), or a nucleic acid sequence that hybridizes to a nucleic acid sequence consisting of SEQ ID NO:98 under stringent hybridization conditions.

In some embodiments, T-box 4 (TBX4) comprises the amino acid sequence: MLQDKGLSESEEAFRAPGPALGEASAANAPEPALAAPGLSGAALGSPPGPGADVVAAAAA EQTIENIKVGLHEKELWKKFHEAGTEMIITKAGRRMFPSYKVKVTGMNPKTKYILLIDIVPAD DHRYKFCDNKWMVAGKAEPAMPGRLYVHPDSPATGAHWMRQLVSFQKLKLTNNHLDPF GHIILNSMHKYQPRLHIVKADENNAFGSKNTAFCTHVFPETSFISVTSYQNHKITQLKIENNP FAKGFRGSDDSDLRVARLQSKEYPVISKSIMRQRLISPQLSATPDVGPLLGTHQALQHYQH ENGAHSQLAEPQDLPLSTFPTQRDSSLFYHCLKRRDGTRHLDLPCKRSYLEAPSSVGEDH YFRSPPPYDQQMLSPSYCSEVTPREACMYSGSGPEIAGVSGVDDLPPPPLSCNMWTSVS PYTSYSVQTMETVPYQPFPTHFTATTMMPRLPTLSAQSSQPPGNAHFSVYNQLSQSQVRE RGPSASFPRERGLPQGCERKPPSPHLNAANEFLYSQTFSLSRESSLQYHSGMGTVENWT DG (SEQ ID NO:99; NP_060958), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:99.

In some embodiments, the nucleic acid sequence encoding TBX4 comprises the nucleic acid sequence: ATGCTGCAGGATAAGGGCCTGTCCGAGAGCGAGGAGGCCTTCCGGGCCCCGGGCCC AGCGCTCGGAGAGGCCAGCGCAGCCAACGCCCCCGAGCCCGCGCTGGCAGCGCCGG GCCTCAGCGGAGCCGCGCTAGGCAGCCCCCCGGGACCCGGGGCCGACGTCGTCGCC GCCGCCGCCGCGGAGCAGACCATCGAGAACATCAAGGTGGGGCTGCATGAGAAGGA GCTCTGGAAGAAGTTCCACGAGGCGGGCACCGAGATGATCATCACTAAGGCTGGCAG GAGGATGTTCCCCAGCTACAAGGTAAAAGTCACAGGCATGAACCCCAAGACCAAGTAT ATCCTGCTGATTGACATTGTCCCTGCCGATGACCATCGCTACAAGTTCTGTGACAACAA ATGGATGGTGGCAGGGAAGGCTGAGCCAGCCATGCCAGGAAGGCTGTATGTCCACCC GGATTCTCCTGCCACAGGAGCCCACTGGATGCGGCAGCTGGTCTCCTTCCAGAAGCT GAAGCTGACAAACAACCACCTGGACCCCTTTGGCCATATCATCCTCAACTCTATGCACA AGTACCAGCCGCGGCTCCACATCGTTAAGGCTGATGAGAACAATGCTTTCGGCTCCAA AAACACTGCTTTCTGCACCCACGTGTTCCCAGAGACCTCCTTCATCTCTGTGACCTCCT ACCAGAATCACAAGATCACCCAGCTGAAAATTGAGAACAACCCTTTTGCCAAGGGATTC CGGGGCAGTGATGACAGTGACCTGCGTGTGGCCCGACTGCAGAGCAAAGAATACCCC GTGATTTCCAAAAGCATCATGAGGCAGAGGCTCATCTCCCCCCAGCTCTCAGCCACAC CGGACGTGGGCCCCCTGCTCGGCACCCACCAGGCACTCCAGCACTACCAGCACGAGA ACGGGGCACACTCACAGCTCGCGGAGCCGCAGGACCTGCCCCTCAGCACCTTTCCCA CCCAGAGGGACTCAAGCCTCTTCTATCACTGCCTGAAAAGACGAGACGGTACCCGCCA CCTGGACTTACCTTGCAAGCGATCCTATCTGGAAGCCCCCTCTTCGGTGGGGGAGGAT CACTATTTCCGTTCCCCCCCTCCCTACGACCAGCAAATGCTGAGCCCCTCCTACTGCA GTGAGGTGACCCCCAGAGAAGCATGTATGTACTCAGGTTCAGGGCCCGAGATTGCCG GGGTGTCTGGGGTGGACGACCTGCCCCCACCTCCGCTGAGCTGTAACATGTGGACTT CAGTGTCGCCGTACACCAGCTATAGCGTGCAGACGATGGAGACTGTGCCGTACCAGC CCTTCCCCACGCACTTCACCGCCACCACCATGATGCCGCGGCTGCCCACCCTCTCCG CTCAGAGCTCCCAGCCACCAGGAAATGCCCACTTTAGTGTCTACAATCAGCTCTCCCA GTCTCAGGTCCGAGAGCGGGGGCCCAGCGCCTCATTCCCAAGAGAGCGCGGCCTCC CCCAAGGGTGTGAGAGGAAGCCACCCTCGCCACATCTAAATGCTGCCAATGAGTTTCT CTACTCTCAAACCTTCTCCTTGTCCCGAGAATCTTCCTTACAGTACCATTCAGGAATGG GGACTGTGGAGAACTGGACTGACGGA (SEQ ID NO:100; NM_018488), or a nucleic acid sequence that hybridizes to a nucleic acid sequence consisting of SEQ ID NO:100 under stringent hybridization conditions.

In some embodiments, T-box 5 (TBX5) comprises the amino acid sequence:

(SEQ ID NO: 101; NP_000183)
MADADEGFGLAHTPLEPDAKDLPCDSKPESALGAPSKSPSSPQAAFTQQ
GMEGIKVFLHERELWLKFHEVGTEMIITKAGRRMFPSYKVKVTGLNPKT
KYILLMDIVPADDHRYKFADNKWSVTGKAEPAMPGRLYVHPDSPATGAH
WMRQLVSFQKLKLTNNHLDPFGHIILNSMHKYQPRLHIVKADENNGFGS
KNTAFCTHVFPETAFIAVTSYQNHKITQLKIENNPFAKGFRGSDDMELH
RMSRMQSKEYPVVPRSTVRQKVASNHSPFSSESRALSTSSNLGSQYQCE
NGVSGPSQDLLPPPNPYPLPQEHSQIYHCTKRKEEECSTTDHPYKKPYM
ETSPSEEDSFYRSSYPQQQGLGASYRTESAQRQACMYASSAPPSEPVPS
LEDISCNTWPSMPSYSSCTVTTVQPMDRLPYQHFSAHFTSGPLVPRLAG
MANHGSPQLGEGMFQHQTSVAHQPVVRQCGPQTGLQSPGTLQPPEFLYS
HGVPRTLSPHQYHSVHGVGMVPEWSDNS,

or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:101.

In some embodiments, the nucleic acid sequence encoding TBX5 comprises the nucleic acid sequence: ATGGCCGACGCAGACGAGGGCTTTGGCCTGGCGCACACGCCTCTGGAGCCTGACGCA AAAGACCTGCCCTGCGATTCGAAACCCGAGAGCGCGCTCGGGGCCCCCAGCAAGTCC CCGTCGTCCCCGCAGGCCGCCTTCACCCAGCAGGGCATGGAGGGAATCAAAGTGTTT CTCCATGAAAGAGAACTGTGGCTAAAATTCCACGAAGTGGGCACGGAAATGATCATAA CCAAGGCTGGAAGGCGGATGTTTCCCAGTTACAAAGTGAAGGTGACGGGCCTTAATCC CAAAACGAAGTACATTCTTCTCATGGACATTGTACCTGCCGACGATCACAGATACAAAT TCGCAGATAATAAATGGTCTGTGACGGGCAAAGCTGAGCCCGCCATGCCTGGCCGCCT GTACGTGCACCCAGACTCCCCCGCCACCGGGGCGCATTGGATGAGGCAGCTCGTCTC CTTCCAGAAACTCAAGCTCACCAACAACCACCTGGACCCATTTGGGCATATTATTCTAA ATTCCATGCACAAATACCAGCCTAGATTACACATCGTGAAAGCGGATGAAAATAATGGA TTTGGCTCAAAAAATACAGCGTTCTGCACTCACGTCTTTCCTGAGACTGCGTTTATAGC AGTGACTTCTTACCAGAACCACAAGATCACGCAATTAAAGATTGAGAATAATCCCTTTG CCAAAGGATTTCGGGGCAGTGATGACATGGAGCTGCACAGAATGTCAAGAATGCAAAG TAAAGAATATCCCGTGGTCCCCAGGAGCACCGTGAGGCAAAAAGTGGCCTCCAACCAC AGTCCTTTCAGCAGCGAGTCTCGAGCTCTCTCCACCTCATCCAATTTGGGGTCCCAATA CCAGTGTGAGAATGGTGTTTCCGGCCCCTCCCAGGACCTCCTGCCTCCACCCAACCCA TACCCACTGCCCCAGGAGCATAGCCAAATTTACCATTGTACCAAGAGGAAAGAGGAAG AATGTTCCACCACAGACCATCCCTATAAGAAGCCCTACATGGAGACATCACCCAGTGAA GAAGATTCCTTCTACCGCTCTAGCTATCCACAGCAGCAGGGCCTGGGTGCCTCCTACA GGACAGAGTCGGCACAGCGGCAAGCTTGCATGTATGCCAGCTCTGCGCCCCCCAGCG AGCCTGTGCCCAGCCTAGAGGACATCAGCTGCAACACGTGGCCAAGCATGCCTTCCTA CAGCAGCTGCACCGTCACCACCGTGCAGCCCATGGACAGGCTACCCTACCAGCACTT CTCCGCTCACTTCACCTCGGGGCCCCTGGTCCCTCGGCTGGCTGGCATGGCCAACCA TGGCTCCCCACAGCTGGGAGAGGGAATGTTCCAGCACCAGACCTCCGTGGCCCACCA GCCTGTGGTCAGGCAGTGTGGGCCTCAGACTGGCCTGCAGTCCCCTGGCACCCTTCA GCCCCCTGAGTTCCTCTACTCTCATGGCGTGCCAAGGACTCTATCCCCTCATCAGTAC CACTCTGTGCACGGAGTTGGCATGGTGCCAGAGTGGAGCGACAATAGC (SEQ ID NO:102; NM_000192), or a nucleic acid sequence that hybridizes to a nucleic acid sequence consisting of SEQ ID NO:102 under stringent hybridization conditions.

In some embodiments, T-box 6 (TBX6) comprises the amino acid sequence: MYHPRELYPSLGAGYRLGPAQPGADSSFPPALAEGYRYPELDTPKLDCFLSGMEAAPRTL AAHPPLPLLPPAMGTEPAPSAPEALHSLPGVSLSLENRELWKEFSSVGTEMIITKAGRRMF PACRVSVTGLDPEARYLFLLDVIPVDGARYRWQGRRWEPSGKAEPRLPDRVYIHPDSPAT GAHWMRQPVSFHRVKLTNSTLDPHGHLILHSMHKYQPRIHLVRAAQLCSQHWGGMASFR FPETTFISVTAYQNPQITQLKIAANPFAKGFRENGRNCKRWELFIHLFMHSTNVY (SEQ ID NO:103; NP_542936), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:103.

In some embodiments, the nucleic acid sequence encoding TBX6 comprises the nucleic acid sequence: ATGTACCATCCACGAGAATTGTACCCGTCCCTGGGGGCCGGCTACCGCCTGGGGCCC GCCCAACCTGGGGCCGACTCCAGCTTCCCACCCGCCCTAGCGGAGGGCTACCGCTAC CCCGAACTGGACACCCCTAAACTGGATTGCTTCCTCTCCGGGATGGAGGCTGCTCCCC GCACCCTGGCCGCGCACCCACCTCTGCCCCTTCTGCCCCCTGCCATGGGCACTGAGC CGGCCCCATCAGCTCCAGAGGCCCTCCATTCCCTCCCGGGGGTCAGCCTGAGCCTGG AGAACCGGGAGCTATGGAAGGAGTTCAGCTCTGTGGGAACAGAAATGATCATCACCAA AGCTGGGAGGCGCATGTTCCCTGCCTGCCGAGTGTCAGTCACTGGCCTGGACCCCGA GGCCCGCTACTTGTTTCTTCTGGATGTGATTCCGGTGGATGGGGCTCGCTACCGCTGG CAGGGCCGGCGCTGGGAGCCCAGCGGCAAGGCAGAGCCCCGCCTGCCTGACCGTGT CTACATTCACCCCGACTCTCCTGCCACTGGTGCACATTGGATGCGGCAGCCTGTGTCT TTCCATCGTGTCAAGCTCACCAACAGCACGCTGGACCCCCACGGCCACCTGATCCTGC ACTCCATGCACAAGTACCAACCCCGCATACACCTAGTTCGGGCAGCCCAGCTCTGCAG CCAGCACTGGGGGGGCATGGCCTCCTTCCGCTTCCCCGAGACCACATTCATCTCCGT GACAGCCTACCAGAACCCACAGATCACACAACTGAAGATTGCAGCCAATCCCTTTGCC AAAGGCTTCCGGGAGAACGGCAGAAACTGTAAGAGGTGGGAGTTGTTCATTCATTTGT TCATGCATTCAACAAATGTTTAT (SEQ ID NO:104; NM_080758), or a nucleic acid sequence that hybridizes to a nucleic acid sequence consisting of SEQ ID NO:104 under stringent hybridization conditions.

In some embodiments, T-box 10 (TBX10) comprises the amino acid sequence: MAAFLSAGLGILAPSETYPLPTTSSGWEPRLGSPFPSGPCTSSTGAQAVAEPTGQGPKNP RVSRVTVQLEMKPLWEEFNQLGTEMIVTKAGRRMFPPFQVKILGMDSLADYALLMDFIPLD DKRYRYAFHSSAWLVAGKADPATPGRVHFHPDSPAKGAQWMRQIVSFDKLKLTNNLLDD NGHIILNSMHRYQPRFHVVFVDPRKDSERYAQENFKSFIFTETQFTAVTAYQNHRITQLKIAS NPFAKGFRESDLDSWPVAPRPLLSVPARSHSSLSPCVLKGATDREKDPNKASASTSKTPA WLHHQLLPPPEVLLAPATYRPVTYQSLYSGAPSHLGIPRTRPAPYPLPNIRADRDQGGLPL PAGLGLLSPTVVCLGPGQDSQ (SEQ ID NO:105; NP_005986), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:105.

In some embodiments, the nucleic acid sequence encoding TBX10 comprises the nucleic acid sequence: ATGGCAGCCTTCCTATCTGCTGGCCTCGGCATACTTGCACCCTCAGAGACCTACCCCC TACCTACAACCAGCTCTGGCTGGGAGCCCCGGCTGGGGTCACCATTCCCATCAGGCC CTTGCACCAGCTCTACTGGGGCCCAAGCTGTGGCCGAGCCCACTGGGCAGGGCCCCA AGAACCCACGTGTGTCCAGAGTGACAGTTCAGCTGGAGATGAAGCCTCTGTGGGAGG AATTCAACCAGCTGGGCACTGAGATGATCGTCACCAAGGCAGGCAGGAGGATGTTCCC CCCCTTCCAGGTGAAGATCCTGGGCATGGACTCCCTGGCCGACTACGCCCTGCTCATG GACTTCATCCCCCTGGACGACAAGAGATACAGGTATGCCTTCCACAGCTCGGCCTGGC TGGTGGCGGGCAAGGCAGACCCAGCCACACCTGGCCGCGTGCACTTCCACCCCGACT CGCCAGCCAAGGGTGCCCAGTGGATGCGCCAGATTGTGTCCTTTGACAAGCTCAAGCT GACCAACAACCTGCTGGATGACAATGGCCACATCATTCTCAACTCTATGCACCGCTACC AGCCCCGTTTCCACGTGGTCTTCGTGGACCCACGCAAGGACAGTGAGCGCTATGCCC AGGAGAACTTCAAGTCCTTCATCTTCACAGAGACCCAGTTCACAGCAGTGACAGCCTAT CAGAACCACAGGATCACCCAGCTGAAAATCGCCAGCAACCCTTTTGCCAAAGGCTTTA GAGAGAGTGACCTGGACTCCTGGCCTGTGGCCCCACGGCCCCTGCTCAGTGTCCCAG CCCGGAGTCACAGCAGCCTCAGTCCCTGTGTGCTGAAGGGTGCCACAGACAGGGAGA AAGACCCCAACAAAGCTTCAGCTTCCACCTCCAAGACCCCTGCTTGGCTCCATCATCA GCTGCTGCCCCCACCTGAGGTCCTGCTGGCCCCGGCCACCTACAGGCCTGTCACGTA TCAGAGCCTGTACTCTGGAGCCCCGAGCCACCTAGGGATCCCAAGGACCCGACCAGC ACCATACCCCCTCCCCAACATCCGGGCTGATAGGGATCAAGGAGGCCTGCCTCTCCCA GCTGGGCTGGGGCTCCTGTCCCCCACTGTGGTGTGCCTGGGGCCTGGCCAGGACTCC CAG (SEQ ID NO:106; NM_005995), or a nucleic acid sequence that hybridizes to a nucleic acid sequence consisting of SEQ ID NO:106 under stringent hybridization conditions.

In some embodiments, T-box 15 (TBX15) comprises the amino acid sequence: MSSMEEIQVELQCADLWKRFHDIGTEMIITKAGRRMFPAMRVKITGLDPHQQYYIAMDIVPV DNKRYRYVYHSSKWMVAGNADSPVPPRVYIHPDSLASGDTWMRQVVSFDKLKLTNNELD DQGHIILHSMHKYQPRVHVIRKDFSSDLSPTKPVPVGDGVKTFNFPETVFTTVTAYQNQQIT RLKIDRNPFAKGFRDSGRNRTGLEAIMETYAFWRPPVRTLTFEDFTTMQKQQGGSTGTSP TTSSTGTPSPSASSHLLSPSCSPPTFHLAPNTFNVGCRESQLCNLNLSDYPPCARSNMAAL QSYPGLSDSGYNRLQSGTTSATQPSETFMPQRTPSLISGIPTPPSLPGNSKMEAYGGQLG SFPTSQFQYVMQAGNAASSSSSPHMFGGSHMQQSSYNAFSLHNPYNLYGYNFPTSPRLA ASPEKLSASQSTLLCSSPSNGAFGERQYLPSGMEHSMHMISPSPNNQQATNTCDGRQYG AVPGSSSQMSVHMV (SEQ ID NO:107; NP_689593), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:107.

In some embodiments, the nucleic acid sequence encoding TBX15 comprises the nucleic acid sequence: ATGTCTTCCATGGAGGAGATTCAGGTGGAGCTGCAATGTGCTGACCTCTGGAAGCGGT TCCATGATATTGGAACTGAAATGATCATCACCAAAGCAGGCAGGAGGATGTTTCCTGCC ATGAGAGTGAAAATCACTGGCCTAGATCCACATCAGCAGTACTACATAGCAATGGACAT TGTGCCTGTGGACAATAAAAGATACAGATATGTGTATCATAGCTCCAAGTGGATGGTGG CTGGCAATGCTGATTCCCCTGTGCCCCCAAGAGTTTATATACACCCTGATTCTCTAGCT TCTGGAGACACCTGGATGAGACAGGTGGTCAGTTTTGACAAACTCAAGCTTACCAACAA TGAGTTGGATGATCAAGGACATATCATTCTGCACTCTATGCACAAATACCAGCCTCGAG TTCATGTGATTCGCAAAGACTTCAGCAGTGACCTTTCACCCACTAAGCCTGTTCCTGTT GGGGATGGGGTGAAAACGTTCAACTTTCCTGAGACTGTGTTCACCACAGTTACGGCCT ATCAGAATCAGCAGATTACCAGATTAAAAATTGACCGAAACCCTTTTGCTAAAGGATTCA GAGATTCTGGGAGAAACAGAACTGGACTTGAAGCCATCATGGAGACATATGCATTCTG GAGACCTCCTGTGCGCACACTCACCTTCGAAGACTTCACCACCATGCAGAAGCAGCAA GGAGGCAGCACAGGCACTTCCCCAACCACCTCCAGCACTGGGACACCATCCCCTTCG GCTTCTTCTCATCTTTTATCTCCATCCTGTTCTCCTCCAACTTTTCATCTGGCCCCCAAC ACTTTCAATGTGGGCTGCCGAGAAAGCCAGCTGTGTAATCTAAACCTCTCTGATTATCC ACCATGTGCCCGAAGCAACATGGCTGCCTTGCAGAGCTACCCAGGGCTGAGTGACAG TGGCTACAACAGGCTTCAGAGTGGCACCACTTCAGCCACTCAGCCCTCTGAAACCTTC ATGCCTCAGAGGACTCCATCCCTGATCTCAGGAATACCAACTCCTCCCTCGTTGCCTG GCAACAGCAAGATGGAAGCCTACGGTGGCCAGCTGGGGTCCTTTCCCACTTCCCAGTT TCAGTATGTCATGCAGGCAGGCAATGCTGCCTCCAGCTCCTCATCACCACACATGTTC GGGGGCAGCCACATGCAGCAGAGCTCCTACAATGCCTTCTCCCTTCACAACCCTTACA ACCTGTATGGATACAATTTCCCCACTTCCCCTAGGCTAGCTGCAAGCCCGGAAAAACTG AGCGCCTCTCAAAGCACTTTACTCTGTTCTTCTCCTTCCAACGGGGCCTTTGGAGAGAG GCAGTACCTGCCGTCAGGGATGGAGCACAGCATGCACATGATTAGTCCTTCACCCAAT AACCAACAGGCAACCAACACTTGTGATGGCCGGCAGTATGGGGCAGTTCCAGGCTCCT CCTCCCAGATGTCCGTGCACATGGTT (SEQ ID NO:108; NM_152380), or a nucleic acid sequence that hybridizes to a nucleic acid sequence consisting of SEQ ID NO:108 under stringent hybridization conditions.

In some embodiments, T-box 18 (TBX18) comprises the amino acid sequence: MAEKRRGSPCSMLSLKAHAFSVEALIGAEKQQQLQKKRRKLGAEEAARAVDDGGCSRGG GAGEKGSSEGDEGAALPPPAGATSGPARSGADLERGAAGGCEDGFQQGASPLASPGGS PKGSPARSLARPGTPLPSPQAPRVDLQGAELWKRFHEIGTEMIITKAGRRMFPAMRVKISG LDPHQQYYIAMDIVPVDNKRYRYVYHSSKWMVAGNADSPVPPRVYIHPDSPASGETWMR QVISFDKLKLTNNELDDQGHIILHSMHKYQPRVHVIRKDCGDDLSPIKPVPSGEGVKAFSFP ETVFTTVTAYQNQQITRLKIDRNPFAKGFRDSGRNRMGLEALVESYAFWRPSLRTLTFEDIP GIPKQGNASSSTLLQGTGNGVPATHPHLLSGSSCSSPAFHLGPNTSQLCSLAPADYSACA RSGLTLNRYSTSLAETYNRLTNQAGETFAPPRTPSYVGVSSSTSVNMSMGGTDGDTFSCP QTSLSMQISGMSPQLQYIMPSPSSNAFATNQTHQGSYNTFRLHSPCALYGYNFSTSPKLAA SPEKIVSSQGSFLGSSPSGTMTDRQMLPPVEGVHLLSSGGQQSFFDSRTLGSLTLSSSQV SAHMV (SEQ ID NO:109; NP_001073977), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:109.

In some embodiments, the nucleic acid sequence encoding TBX18 comprises the nucleic acid sequence ATGGCCGAGAAGCGAAGGGGCTCGCCGTGCAGCATGCTAAGCCTCAAGGCGCACGCT TTCTCGGTGGAGGCGCTGATCGGCGCCGAGAAGCAGCAACAGCTTCAGAAGAAGCGG CGAAAACTGGGCGCCGAAGAGGCGGCGAGGGCCGTGGACGACGGAGGCTGCAGCCG CGGCGGCGGCGCGGGCGAAAAGGGTTCTTCTGAGGGAGACGAAGGCGCTGCGCTCC CGCCGCCGGCTGGGGCGACGTCTGGGCCGGCTCGGAGTGGCGCAGACCTGGAGCG CGGAGCCGCGGGCGGCTGTGAGGACGGCTTCCAGCAGGGAGCTTCCCCTCTGGCGT CACCGGGAGGCTCCCCCAAGGGGTCTCCGGCGCGCTCCCTGGCCCGGCCCGGGACC CCTCTGCCCTCGCCGCAGGCCCCGCGGGTGGATCTGCAGGGAGCCGAGCTCTGGAA GCGCTTTCATGAGATAGGCACTGAGATGATCATCACCAAGGCCGGCAGGCGCATGTTT CCAGCAATGAGAGTGAAGATCTCTGGATTAGATCCTCACCAGCAATATTACATTGCCAT GGATATTGTACCAGTGGACAACAAAAGATACAGGTATGTTTACCACAGTTCGAAATGGA TGGTGGCAGGTAATGCTGACTCGCCTGTGCCACCCCGTGTGTACATTCATCCAGACTC GCCTGCCTCGGGGGAGACTTGGATGAGACAAGTTATCAGCTTCGACAAGCTGAAGCTC ACCAACAATGAACTGGATGACCAAGGCCATATTATTCTTCATTCTATGCACAAATACCAA CCGCGAGTGCACGTCATCCGTAAAGACTGTGGAGACGATCTTTCTCCCATCAAGCCTG TTCCATCCGGGGAGGGAGTAAAGGCATTCTCCTTTCCAGAAACTGTCTTCACAACCGTC ACTGCCTATCAGAATCAGCAGATTACTCGCCTGAAGATAGATAGGAATCCATTTGCTAA AGGCTTCCGAGACTCCGGGCGCAACAGAATGGGTTTGGAAGCCTTGGTGGAATCATAT GCATTCTGGCGACCATCACTACGGACTCTGACCTTTGAAGATATCCCTGGAATTCCCAA GCAAGGCAATGCAAGTTCCTCCACCTTGCTCCAAGGTACTGGGAATGGCGTTCCTGCC ACTCACCCTCACCTTTTGTCTGGCTCCTCTTGCTCCTCTCCTGCCTTCCATCTGGGGCC CAACACCAGCCAGCTGTGTAGTCTGGCCCCTGCTGACTATTCTGCCTGTGCCCGCTCA GGCCTCACCCTCAACCGATACAGCACATCTTTGGCAGAGACCTACAACAGGCTCACCA ACCAGGCTGGTGAGACCTTTGCCCCGCCCAGGACTCCCTCCTATGTGGGCGTGAGCA GCAGCACCTCCGTGAACATGTCCATGGGTGGCACTGATGGGGACACCTTCAGCTGCC CACAGACCAGCTTATCCATGCAGATTTCGGGAATGTCCCCCCAGCTCCAGTATATCATG CCATCACCCTCCAGCAATGCCTTCGCCACTAACCAGACCCATCAGGGTTCCTATAATAC TTTTAGATTACACAGCCCCTGTGCACTATATGGATATAACTTCTCCACATCCCCCAAACT GGCTGCCAGTCCTGAGAAAATTGTTTCTTCCCAAGGAAGTTTCTTGGGGTCCTCACCGA GTGGGACCATGACGGATCGGCAGATGTTGCCCCCTGTGGAAGGAGTGCACCTGCTTA GCAGTGGGGGTCAGCAGAGTTTCTTTGACTCTAGGACCCTAGGAAGCTTAACTCTGTC ATCATCTCAAGTATCTGCACATATGGTC (SEQ ID NO:110; NM_001080508), or a nucleic acid sequence that hybridizes to a nucleic acid sequence consisting of SEQ ID NO:110 under stringent hybridization conditions.

In some embodiments, T-box 10 (TBX19) comprises the amino acid sequence: MAMSELGTRKPSDGTVSHLLNVVESELQAGREKGDPTEKQLQIILEDAPLWQRFKEVTNE MIVTKNGRRMFPVLKISVTGLDPNAMYSLLLDFVPTDSHRWKYVNGEWVPAGKPEVSSHS CVYIHPDSPNFGAHWMKAPISFSKVKLTNKLNGGGQIMLNSLHKYEPQVHIVRVGSAHRMV TNCSFPETQFIAVTAYQNEEITALKIKYNPFAKAFLDAKERNHLRDVPEAISESQHVTYSHLG GWIFSNPDGVCTAGNSNYQYAAPLPLPAPHTHHGCEHYSGLRGHRQAPYPSAYMHRNHS PSVNLIESSSNNLQVFSGPDSWTSLSSTPHASILSVPHTNGPINPGPSPYPCLWTISNGAGG PSGPGPEVHASTPGAFLLGNPAVTSPPSVLSTQAPTSAGVEVLGEPSLTSIAVSTWTAVAS HPFAGWGGPGAGGHHSPSSLDG (SEQ ID NO:111; NP_005140), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:111.

In some embodiments, the nucleic acid sequence encoding TBX19 comprises the nucleic acid sequence: ATGGCCATGAGTGAGCTGGGCACTCGGAAGCCCAGCGATGGCACTGTTTCTCATCTGC TCAATGTGGTGGAGAGTGAGCTTCAGGCAGGGAGGGAAAAAGGCGACCCTACGGAGA AGCAACTTCAGATCATCCTGGAGGATGCACCTCTCTGGCAGAGATTCAAGGAAGTCAC TAATGAGATGATTGTGACCAAGAATGGCAGACGGATGTTTCCAGTCCTAAAGATTAGTG TCACAGGGTTGGACCCCAATGCCATGTACTCCCTCCTGCTGGACTTTGTCCCTACGGA CAGTCACCGCTGGAAGTACGTCAACGGGGAATGGGTGCCCGCTGGCAAGCCAGAGGT CTCCAGCCACAGCTGCGTCTACATTCACCCGGACTCCCCCAACTTTGGGGCCCACTGG ATGAAAGCTCCCATCTCCTTCAGCAAAGTGAAGCTGACCAACAAGCTCAATGGAGGCG GGCAGATAATGTTGAATTCTCTGCATAAATATGAACCCCAGGTTCACATAGTGCGTGTT GGAAGTGCCCATCGAATGGTAACAAACTGCTCCTTCCCTGAAACCCAGTTCATAGCCG TGACTGCCTATCAGAATGAGGAGATAACGGCTCTCAAAATCAAGTACAATCCTTTTGCC AAAGCCTTCTTGGATGCCAAGGAAAGAAATCACCTAAGAGACGTACCGGAGGCTATCT CTGAGAGCCAGCATGTGACCTATTCTCACTTGGGAGGCTGGATCTTTTCCAATCCAGAT GGAGTGTGCACAGCAGGAAACTCCAATTACCAGTATGCCGCTCCTCTGCCTCTGCCTG CTCCCCACACCCACCATGGCTGTGAGCACTATTCGGGTCTCCGAGGACACCGGCAGG CTCCCTACCCTTCTGCGTACATGCACAGAAACCATTCTCCCTCAGTGAATTTGATAGAA AGCTCAAGCAATAATCTGCAAGTTTTCTCGGGACCTGACAGCTGGACTTCCTTATCCTC CACACCCCATGCCAGCATCCTGTCTGTACCCCACACCAACGGACCAATCAATCCAGGG CCCAGCCCCTACCCGTGCCTGTGGACCATCAGCAATGGTGCCGGAGGCCCCAGTGGG CCAGGCCCGGAGGTGCACGCCAGCACCCCAGGAGCATTTCTCCTCGGAAACCCAGCT GTGACTTCACCCCCTTCTGTGCTCTCCACCCAAGCACCCACTTCGGCTGGTGTGGAGG TTCTGGGGGAGCCCTCGCTAACCAGCATTGCTGTGTCCACCTGGACAGCAGTGGCCTC GCATCCCTTCGCGGGCTGGGGTGGCCCAGGAGCGGGTGGGCACCATTCTCCTTCCTC ACTGGATGGT (SEQ ID NO:112; NM_005149), or a nucleic acid sequence that hybridizes to a nucleic acid sequence consisting of SEQ ID NO:112 under stringent hybridization conditions.

In some embodiments, T-box 20 (TBX20) comprises the amino acid sequence: MEFTASPKPQLSSRANAFSIAALMSSGGSKEKEATENTIKPLEQFVEKSSCAQPLGELTSLD AHGEFGGGSGSSPSSSSLCTEPLIPTTPIIPSEEMAKIACSLETKELWDKFHELGTEMIITKS GRRMFPTIRVSFSGVDPEAKYIVLMDIVPVDNKRYRYAYHRSSWLVAGKADPPLPARLYVH PDSPFTGEQLLKQMVSFEKVKLTNNELDQHGHIILNSMHKYQPRVHIIKKKDHTASLLNLKS EEFRTFIFPETVFTAVTAYQNQLITKLKIDSNPFAKGFRDSSRLTDIER (SEQ ID NO:113; NP_001159692), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:113.

In some embodiments, the nucleic acid sequence encoding TBX20 comprises the nucleic acid sequence: ATGGAGTTCACGGCGTCCCCCAAGCCCCAACTCTCCTCCCGGGCCAACGCCTTCTCCA TTGCCGCGCTCATGTCGAGCGGCGGCTCTAAGGAGAAGGAGGCGACGGAGAACACAA TCAAACCCCTGGAGCAATTTGTGGAGAAGTCGTCCTGTGCCCAGCCCCTGGGTGAGCT GACCAGCCTGGATGCTCATGGGGAGTTTGGTGGAGGCAGTGGCAGCAGCCCGTCCTC CTCCTCTCTGTGCACTGAGCCACTGATCCCCACCACCCCCATCATCCCCAGTGAGGAA ATGGCCAAAATTGCCTGCAGCCTGGAGACCAAGGAGCTTTGGGACAAATTCCATGAGC TGGGCACCGAGATGATCATCACCAAGTCGGGCAGGAGGATGTTTCCAACCATCCGGGT GTCCTTTTCGGGGGTGGATCCTGAGGCCAAGTACATAGTCCTGATGGACATCGTCCCT GTGGACAACAAGAGGTACCGCTACGCCTACCACCGGTCCTCCTGGCTGGTGGCTGGC AAGGCCGACCCGCCGTTGCCAGCCAGGCTCTATGTGCATCCAGATTCTCCTTTTACCG GTGAGCAACTACTCAAACAGATGGTGTCTTTTGAAAAGGTGAAACTCACCAACAATGAA CTGGATCAACATGGCCATATAATTTTGAACTCAATGCATAAGTACCAGCCAAGGGTGCA CATCATTAAGAAGAAAGACCACACAGCCTCATTGCTCAACCTGAAGTCTGAAGAATTTA GAACTTTCATCTTTCCAGAAACAGTTTTTACGGCAGTCACTGCCTACCAGAATCAACTG ATAACGAAGCTGAAAATAGATAGCAATCCTTTTGCCAAAGGATTCCGGGATTCCTCCAG GCTCACTGACATTGAGAGGT (SEQ ID NO:114; NM_001166220), or a nucleic acid sequence that hybridizes to a nucleic acid sequence consisting of SEQ ID NO: 114 under stringent hybridization conditions.

In some embodiments, T-box 21 (TBX21) comprises the amino acid sequence: MGIVEPGCGDMLTGTEPMPGSDEGRAPGADPQHRYFYPEPGAQDADERRGGGSLGSPY PGGALVPAPPSRFLGAYAYPPRPQAAGFPGAGESFPPPADAEGYQPGEGYAAPDPRAGL YPGPREDYALPAGLEVSGKLRVALNNHLLWSKFNQHQTEMIITKQGRRMFPFLSFTVAGLE PTSHYRMFVDVVLVDQHHWRYQSGKWVQCGKAEGSMPGNRLYVHPDSPNTGAHWMRQ EVSFGKLKLTNNKGASNNVTQMIVLQSLHKYQPRLHIVEVNDGEPEAACNASNTHIFTFQET QFIAVTAYQNAEITQLKIDNNPFAKGFRENFESMYTSVDTSIPSPPGPNCQFLGGDHYSPLL PNQYPVPSRFYPDLPGQAKDVVPQAYWLGAPRDHSYEAEFRAVSMKPAFLPSAPGPTMS YYRGQEVLAPGAGWPVAPQYPPKMGPASWFRPMRTLPMEPGPGGSEGRGPEDQGPPL VWTEIAPIRPESSDSGLGEGDSKRRRVSPYPSSGDSSSPAGAPSPFDKEAEGQFYNYFPN (SEQ ID NO: 115; NP_037483), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 115.

In some embodiments, the nucleic acid sequence encoding TBX21 comprises the nucleic acid sequence: ATGGGCATCGTGGAGCCGGGTTGCGGAGACATGCTGACGGGCACCGAGCCGATGCC GGGGAGCGACGAGGGCCGGGCGCCTGGCGCCGACCCGCAGCACCGCTACTTCTACC CGGAGCCGGGCGCGCAGGACGCGGACGAGCGTCGCGGGGGCGGCAGCCTGGGGTC TCCCTACCCGGGGGGCGCCTTGGTGCCCGCCCCGCCGAGCCGCTTCCTTGGAGCCTA CGCCTACCCGCCGCGACCCCAGGCGGCCGGCTTCCCCGGCGCGGGCGAGTCCTTCC CGCCGCCCGCGGACGCCGAGGGCTACCAGCCGGGCGAGGGCTACGCCGCCCCGGA CCCGCGCGCCGGGCTCTACCCGGGGCCGCGTGAGGACTACGCGCTACCCGCGGGAC TGGAGGTGTCGGGGAAACTGAGGGTCGCGCTCAACAACCACCTGTTGTGGTCCAAGTT TAATCAGCACCAGACAGAGATGATCATCACCAAGCAGGGACGGCGGATGTTCCCATTC CTGTCATTTACTGTGGCCGGGCTGGAGCCCACCAGCCACTACAGGATGTTTGTGGACG TGGTCTTGGTGGACCAGCACCACTGGCGGTACCAGAGCGGCAAGTGGGTGCAGTGTG GAAAGGCCGAGGGCAGCATGCCAGGAAACCGCCTGTACGTCCACCCGGACTCCCCCA ACACAGGAGCGCACTGGATGCGCCAGGAAGTTTCATTTGGGAAACTAAAGCTCACAAA CAACAAGGGGGCGTCCAACAATGTGACCCAGATGATTGTGCTCCAGTCCCTCCATAAG TACCAGCCCCGGCTGCATATCGTTGAGGTGAACGACGGAGAGCCAGAGGCAGCCTGC AACGCTTCCAACACGCATATCTTTACTTTCCAAGAAACCCAGTTCATTGCCGTGACTGC CTACCAGAATGCCGAGATTACTCAGCTGAAAATTGATAATAACCCCTTTGCCAAAGGAT TCCGGGAGAACTTTGAGTCCATGTACACATCTGTTGACACCAGCATCCCCTCCCCGCC TGGACCCAACTGTCAATTCCTTGGGGGAGATCACTACTCTCCTCTCCTACCCAACCAGT ATCCTGTTCCCAGCCGCTTCTACCCCGACCTTCCTGGCCAGGCGAAGGATGTGGTTCC CCAGGCTTACTGGCTGGGGGCCCCCCGGGACCACAGCTATGAGGCTGAGTTTCGAGC AGTCAGCATGAAGCCTGCATTCTTGCCCTCTGCCCCTGGGCCCACCATGTCCTACTAC CGAGGCCAGGAGGTCCTGGCACCTGGAGCTGGCTGGCCTGTGGCACCCCAGTACCCT CCCAAGATGGGCCCGGCCAGCTGGTTCCGCCCTATGCGGACTCTGCCCATGGAACCC GGCCCTGGAGGCTCAGAGGGACGGGGACCAGAGGACCAGGGTCCCCCCTTGGTGTG GACTGAGATTGCCCCCATCCGGCCGGAATCCAGTGATTCAGGACTGGGCGAAGGAGA CTCTAAGAGGAGGCGCGTGTCCCCCTATCCTTCCAGTGGTGACAGCTCCTCCCCTGCT GGGGCCCCTTCTCCTTTTGATAAGGAAGCTGAAGGACAGTTTTATAACTATTTTCCCAA C (SEQ ID NO:116; NM_013351), or a nucleic acid sequence that hybridizes to a nucleic acid sequence consisting of SEQ ID NO: 116 under stringent hybridization conditions.

In some embodiments, T-box 22 (TBX22) comprises the amino acid sequence: MALSSRARAFSVEALVGRPSKRKLQDPIQAEQPELREKKGGEEEEERRSSAAGKSEPLEK QPKTEPSTSASSGCGSDSGYGNSSESLEEKDIQMELQGSELWKRFHDIGTEMIITKAGRRM FPSVRVKVKGLDPGKQYHVAIDVVPVDSKRYRYVYHSSQWMVAGNTDHLCllPRFYVHPD SPCSGETWMRQIISFDRMKLTNNEMDDKGHIlLQSMHKYKPRVHVIEQGSSVDLSQIQSLPT EGVKTFSFKETEFTTVTAYQNQQITKLKIERNPFAKGFRDTGRNRGVLDGLLETYPWRPSF TLDFKTFGADTQSGSSGSSPVTSSGGAPSPLNSLLSPLCFSPMFHLPTSSLGMPCPEAYLP NVNLPLCYKICPTNFWQQQPLVLPAPERLASSNSSQSLAPLMMEVPMLSSLGVTNSKSGS SEDSSDQYLQAPNSTNQMLYGLQSPGNIFLPNSITPEALSCSFHPSYDFYRYNFSMPSRLIS GSNHLKVNDDSQVSFGEGKCNHVHWYPAINHYL (SEQ ID NO: 117; NP_058650), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:117.

In some embodiments, the nucleic acid sequence encoding TBX22 comprises the nucleic acid sequence:

(SEQ ID NO: 118; NM_016954)
ATGGCTCTGAGCTCTCGGGCGCGTGCCTTCTCCGTGGAAGCCTTGGTGG
GGAGACCCAGCAAAAGAAAACTCCAAGACCCAATACAGGCGGAGCAGCC
TGAGCTGCGGGAGAAAAAGGGCGGAGAGGAAGAGGAGGAGAGAAGGAGC
AGCGCTGCAGGGAAGAGCGAGCCGCTTGAAAAACAACCTAAGACAGAGC
CCTCAACATCTGCTTCCTCTGGCTGCGGCAGCGACAGCGGCTACGGCAA
CAGCTCTGAAAGTCTGGAAGAGAAAGATATTCAAATGGAGCTTCAAGGA
TCTGAACTGTGGAAAAGATTCCATGACATCGGGACTGAGATGATCATTA
CTAAAGCGGGCAGGCGGATGTTCCCCTCTGTTCGGGTCAAGGTGAAAGG
GTTGGATCCAGGGAAGCAGTACCATGTGGCCATCGATGTGGTGCCGGTG
GATTCCAAACGCTATAGGTACGTCTATCACAGCTCACAGTGGATGGTAG
CTGGGAATACAGACCATTTGTGCATCATTCCTAGATTCTATGTTCACCC
GGACTCACCCTGCTCGGGAGAGACCTGGATGCGGCAGATCATCAGCTTT
GATCGCATGAAACTCACCAACAATGAGATGGATGACAAAGGCCACATCA
TTCTGCAATCCATGCATAAGTACAAACCCCGAGTGCACGTGATAGAGCA
AGGCAGCAGTGTTGACCTGTCCCAGATTCAGTCCTTGCCCACTGAAGGT
GTTAAAACATTCTCCTTTAAAGAAACTGAGTTCACCACAGTAACGGCTT
ACCAAAACCAACAGATTACGAAACTAAAAATAGAAAGAAATCCTTTTGC
TAAAGGATTTAGAGATACTGGAAGAAACAGGGGTGTATTGGATGGGCTT
TTAGAGACCTACCCATGGAGGCCTTCTTTCACTCTCGATTTTAAAACCT
TTGGCGCAGACACACAAAGTGGAAGCAGTGGCTCATCTCCAGTGACCTC
TAGTGGAGGGGCCCCCTCTCCTTTGAACTCCTTACTTTCTCCACTTTGC
TTTTCACCTATGTTTCATTTACCTACAAGCTCCCTTGGAATGCCCTGTC
CAGAGGCATACCTGCCCAATGTCAACCTGCCTCTATGCTACAAGATTTG
TCCAACTAATTTTTGGCAACAGCAACCTCTTGTTTTACCGGCTCCTGAA
AGACTAGCAAGCAGCAACAGTTCTCAGTCTTTAGCCCCACTCATGATGG
AAGTGCCTATGTTATCTTCCCTGGGGGTCACCAATTCAAAAAGCGGTTC
ATCTGAAGACTCCAGTGATCAGTATCTACAAGCACCTAATTCTACCAAT
CAAATGTTATATGGATTACAGTCACCTGGAAATATTTTTCTGCCAAACT
CCATCACCCCAGAAGCACTTAGTTGCTCCTTTCATCCTTCCTATGACTT
TTATAGATACAATTTCTCTATGCCATCTAGACTGATAAGTGGTTCCAAC
CATCTTAAAGTGAATGACGACAGTCAAGTTTCTTTTGGAGAAGGCAAAT
GTAATCATGTTCATTGGTATCCAGCAATTAACCATTACCTT,

or a nucleic acid sequence that hybridizes to a nucleic acid sequence consisting of SEQ ID NO:118 under stringent hybridization conditions.

In some embodiments, Paired box 1 (PAX1) comprises the amino acid sequence: MEQTYGEVNQLGGVFVNGRPLPNAIRLRIVELAQLGIRPCDISRQLRVSHGCVSKILARYNE TGSILPGAIGGSKPRVTTPNVVKHIRDYKQGDPGIFAWEIRDRLLADGVCDKYNVPSVSSIS RILRNKIGSLAQPGPYEASKQPPSQPTLPYNHIYQYPYPSPVSPTGAKMGSHPGVPGTAGH VSIPRSWPSAHSVSNILGIRTFMEQTGALAGSEGTAYSPKMEDWAGVNRTAFPATPAVNGL EKPALEADIKYTQSASTLSAVGGFLPACAYPASNQHGVYSAPGGGYLAPGPPWPPAQGPP LAPPGAGVAVHGGELAAAMTFKHPSREGSLPAPAARPRTPSVAYTDCPSRPRPPRGSSPR TRARRERQADPGAQVCAAAPAIGTGRIGGLAEEEASAGPRGARPASPQAQPCLWPDPPH FLYWSGFLGFSELGF (SEQ ID NO:119; NP_006183), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:119.

In some embodiments, the nucleic acid sequence encoding PAX1 comprises the nucleic acid sequence: ATGGAGCAGACGTATGGCGAGGTGAACCAGCTGGGCGGTGTGTTCGTCAACGGCCGC CCCCTGCCCAACGCCATCCGCTTGCGCATTGTGGAGCTGGCGCAGCTGGGCATCCGA CCCTGTGACATCAGTCGGCAGCTCCGCGTATCCCACGGCTGCGTGAGCAAGATCCTG GCGCGCTACAACGAGACCGGCTCCATTCTGCCCGGGGCCATCGGGGGGAGCAAGCC CCGCGTCACCACTCCCAACGTGGTCAAGCACATCCGGGACTACAAGCAAGGAGACCC TGGCATCTTTGCCTGGGAGATCCGCGACCGGCTGCTGGCCGACGGCGTCTGTGACAA GTACAATGTGCCTTCGGTGAGCTCCATCAGCCGCATCCTGCGCAACAAGATCGGCAGC CTGGCGCAGCCCGGACCGTACGAGGCAAGTAAGCAGCCGCCGTCGCAGCCTACGCT GCCCTACAACCACATCTACCAGTACCCCTACCCCAGTCCCGTGTCGCCCACGGGCGC CAAGATGGGCAGCCACCCCGGGGTCCCGGGCACGGCGGGCCACGTCAGCATCCCGC GCTCATGGCCCTCGGCACACTCGGTCAGCAACATCCTGGGCATCCGGACGTTTATGGA GCAAACAGGGGCCCTGGCTGGGAGCGAAGGCACCGCTTACTCTCCCAAGATGGAAGA CTGGGCCGGCGTGAACCGCACGGCCTTCCCCGCCACCCCCGCAGTGAATGGGCTAGA GAAACCTGCCTTAGAGGCAGACATTAAATACACTCAGTCGGCCTCCACCCTCTCTGCC GTGGGCGGCTTTCTCCCCGCCTGCGCCTACCCGGCCTCCAACCAGCACGGCGTGTAC AGCGCCCCGGGCGGCGGCTACCTCGCCCCGGGCCCGCCGTGGCCGCCTGCGCAAG GTCCTCCTCTGGCGCCCCCCGGGGCCGGCGTAGCTGTGCATGGCGGGGAACTCGCG GCAGCAATGACCTTCAAGCATCCCAGCCGAGAAGGAAGCCTCCCAGCTCCGGCAGCA AGGCCCCGGACGCCCTCAGTAGCTTACACGGACTGCCCATCCCGGCCTCGACCTCCT AGGGGCAGCTCTCCCCGGACCCGAGCCCGGAGGGAACGGCAGGCGGACCCGGGCG CACAGGTCTGCGCGGCGGCCCCGGCAATCGGCACGGGCAGGATCGGAGGACTCGCG GAGGAGGAAGCCAGTGCCGGCCCGCGGGGTGCACGCCCAGCCAGCCCCCAGGCCCA GCCCTGCCTCTGGCCGGACCCACCACACTTCCTTTATTGGTCTGGGTTTTTAGGCTTCT CTGAACTTGGGTTT (SEQ ID NO:120; NM_006192), or a nucleic acid sequence that hybridizes to a nucleic acid sequence consisting of SEQ ID NO:120 under stringent hybridization conditions.

In some embodiments, Sex determining region Y (SRY/SOXA) comprises the amino acid sequence: MQSYASAMLSVFNSDDYSPAVQENIPALRRSSSFLCTESCNSKYQCETGENSKGNVQDRV KRPMNAFIVWSRDQRRKMALENPRMRNSEISKQLGYQWKMLTEAEKWPFFQEAQKLQAM HREKYPNYKYRPRRKAKMLPKNCSLLPADPASVLCSEVQLDNRLYRDDCTKATHSRMEHQ LGHLPPINAASSPQQRDRYSHWTKL (SEQ ID NO:121; NP_003131), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:121.

In some embodiments, the nucleic acid sequence encoding SRY/SOXA comprises the nucleic acid sequence: ATGCAATCATATGCTTCTGCTATGTTAAGCGTATTCAACAGCGATGATTACAGTCCAGCT GTGCAAGAGAATATTCCCGCTCTCCGGAGAAGCTCTTCCTTCCTTTGCACTGAAAGCTG TAACTCTAAGTATCAGTGTGAAACGGGAGAAAACAGTAAAGGCAACGTCCAGGATAGA GTGAAGCGACCCATGAACGCATTCATCGTGTGGTCTCGCGATCAGAGGCGCAAGATG GCTCTAGAGAATCCCAGAATGCGAAACTCAGAGATCAGCAAGCAGCTGGGATACCAGT GGAAAATGCTTACTGAAGCCGAAAAATGGCCATTCTTCCAGGAGGCACAGAAATTACA GGCCATGCACAGAGAGAAATACCCGAATTATAAGTATCGACCTCGTCGGAAGGCGAAG ATGCTGCCGAAGAATTGCAGTTTGCTTCCCGCAGATCCCGCTTCGGTACTCTGCAGCG AAGTGCAACTGGACAACAGGTTGTACAGGGATGACTGTACGAAAGCCACACACTCAAG AATGGAGCACCAGCTAGGCCACTTACCGCCCATCAACGCAGCCAGCTCACCGCAGCA ACGGGACCGCTACAGCCACTGGACAAAGCTG (SEQ ID NO:122; NM_003140), or a nucleic acid sequence that hybridizes to a nucleic acid sequence consisting of SEQ ID NO:122 under stringent hybridization conditions.

In some embodiments, SRY box 1 (SOX1) comprises the amino acid sequence: MYSMMMETDLHSPGGAQAPTNLSGPAGAGGGGGGGGGGGGGGGAKANQDRVKRPMN AFMVWSRGQRRKMAQENPKMHNSEISKRLGAEWKVMSEAEKRPFIDEAKRLRALHMKEH PDYKYRPRRKTKTLLKKDKYSLAGGLLAAGAGGGGAAVAMGVGVGVGAAAVGQRLESPG GAAGGGYAHVNGWANGAYPGSVAAAAAAAAMMQEAQLAYGQHPGAGGAHPHAHPAHP HPHHPHAHPHNPQPMHRYDMGALQYSPISNSQGYMSASPSGYGGLPYGAAAAAAAAAG GAHQNSAVAAAAAAAAASSGALGALGSLVKSEPSGSPPAPAHSRAPCPGDLREMISMYLP AGEGGDPAAAAAAAAQSRLHSLPQHYQGAGAGVNGTVPLTHI (SEQ ID NO:123; NP_005977), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:123.

In some embodiments, the nucleic acid sequence encoding SOX1 comprises the nucleic acid sequence: ATGTACAGCATGATGATGGAGACCGACCTGCACTCGCCCGGCGGCGCCCAGGCCCCC ACGAACCTCTCGGGCCCCGCCGGGGCGGGCGGCGGCGGGGGCGGAGGCGGGGGC GGCGGCGGCGGCGGGGGCGCCAAGGCCAACCAGGACCGGGTCAAACGGCCCATGAA CGCCTTCATGGTGTGGTCCCGCGGGCAGCGGCGCAAGATGGCCCAGGAGAACCCCAA GATGCACAACTCGGAGATCAGCAAGCGCCTGGGGGCCGAGTGGAAGGTCATGTCCGA GGCCGAGAAGCGGCCGTTCATCGACGAGGCCAAGCGGCTGCGCGCGCTGCACATGA AGGAGCACCCGGATTACAAGTACCGGCCGCGCCGCAAGACCAAGACGCTGCTCAAGA AGGACAAGTACTCGCTGGCCGGCGGGCTCCTGGCGGCCGGCGCGGGTGGCGGCGG CGCGGCTGTGGCCATGGGCGTGGGCGTGGGCGTGGGCGCGGCGGCCGTGGGCCAG CGCCTGGAGAGCCCAGGCGGCGCGGCGGGCGGCGGCTACGCGCACGTCAACGGCT GGGCCAACGGCGCCTACCCCGGCTCGGTGGCGGCGGCGGCGGCGGCCGCGGCCAT GATGCAGGAGGCGCAGCTGGCCTACGGGCAGCACCCGGGCGCGGGCGGCGCGCAC CCGCACGCGCACCCCGCGCACCCGCACCCGCACCACCCGCACGCGCACCCGCACAA CCCGCAGCCCATGCACCGCTACGACATGGGCGCGCTGCAGTACAGCCCCATCTCCAA CTCGCAGGGCTACATGAGCGCGTCGCCCTCGGGCTACGGCGGCCTCCCCTACGGCG CCGCGGCCGCCGCCGCCGCCGCTGCGGGCGGCGCGCACCAGAACTCGGCCGTGGC GGCGGCGGCGGCGGCGGCGGCCGCGTCGTCGGGCGCCCTGGGCGCGCTGGGCTCT CTGGTGAAGTCGGAGCCCAGCGGCAGCCCGCCCGCCCCAGCGCACTCGCGGGCGCC GTGCCCCGGGGACCTGCGCGAGATGATCAGCATGTACTTGCCCGCCGGCGAGGGGG GCGACCCGGCGGCGGCAGCAGCGGCCGCGGCGCAGAGCCGGCTGCACTCGCTGCC GCAGCACTACCAGGGCGCGGGCGCGGGCGTGAACGGCACGGTGCCCCTGACGCACA TC (SEQ ID NO:124; NM_005986), or a nucleic acid sequence that hybridizes to a nucleic acid sequence consisting of SEQ ID NO:124 under stringent hybridization conditions.

In some embodiments, SRY-box 2 (SOX2) comprises the amino acid sequence: MYNMMETELKPPGPQQTSGGGGGNSTAAAAGGNQKNSPDRVKRPMNAFMVWSRGQRR KMAQENPKMHNSEISKRLGAEWKLLSETEKRPFIDEAKRLRALHMKEHPDYKYRPRRKTKT LMKKDKYTLPGGLLAPGGNSMASGVGVGAGLGAGVNQRMDSYAHMNGWSNGSYSMMQ DQLGYPQHPGLNAHGAAQMQPMHRYDVSALQYNSMTSSQTYMNGSPTYSMSYSQQGTP GMALGSMGSVVKSEASSSPPVVTSSSHSRAPCQAGDLRDMISMYLPGAEVPEPAAPSRLH MSQHYQSGPVPGTAINGTLPLSHM (SEQ ID NO:125; NP_003097), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:125.

In some embodiments, the nucleic acid sequence encoding SOX2 comprises the nucleic acid sequence: ATGTACAACATGATGGAGACGGAGCTGAAGCCGCCGGGCCCGCAGCAAACTTCGGGG GGCGGCGGCGGCAACTCCACCGCGGCGGCGGCCGGCGGCAACCAGAAAAACAGCCC GGACCGCGTCAAGCGGCCCATGAATGCCTTCATGGTGTGGTCCCGCGGGCAGCGGC GCAAGATGGCCCAGGAGAACCCCAAGATGCACAACTCGGAGATCAGCAAGCGCCTGG GCGCCGAGTGGAAACTTTTGTCGGAGACGGAGAAGCGGCCGTTCATCGACGAGGCTA AGCGGCTGCGAGCGCTGCACATGAAGGAGCACCCGGATTATAAATACCGGCCCCGGC GGAAAACCAAGACGCTCATGAAGAAGGATAAGTACACGCTGCCCGGCGGGCTGCTGG CCCCCGGCGGCAATAGCATGGCGAGCGGGGTCGGGGTGGGCGCCGGCCTGGGCGC GGGCGTGAACCAGCGCATGGACAGTTACGCGCACATGAACGGCTGGAGCAACGGCAG CTACAGCATGATGCAGGACCAGCTGGGCTACCCGCAGCACCCGGGCCTCAATGCGCA CGGCGCAGCGCAGATGCAGCCCATGCACCGCTACGACGTGAGCGCCCTGCAGTACAA CTCCATGACCAGCTCGCAGACCTACATGAACGGCTCGCCCACCTACAGCATGTCCTAC TCGCAGCAGGGCACCCCTGGCATGGCTCTTGGCTCCATGGGTTCGGTGGTCAAGTCC GAGGCCAGCTCCAGCCCCCCTGTGGTTACCTCTTCCTCCCACTCCAGGGCGCCCTGC CAGGCCGGGGACCTCCGGGACATGATCAGCATGTATCTCCCCGGCGCCGAGGTGCCG GAACCCGCCGCCCCCAGCAGACTTCACATGTCCCAGCACTACCAGAGCGGCCCGGTG CCCGGCACGGCCATTAACGGCACACTGCCCCTCTCACACATG (SEQ ID NO:126; NM_003106), or a nucleic acid sequence that hybridizes to a nucleic acid sequence consisting of SEQ ID NO:126 under stringent hybridization conditions.

In some embodiments, SRY-box 3 (SOX3) comprises the amino acid sequence: MRPVRENSSGARSPRVPADLARSILISLPFPPDSLAHRPPSSAPTESQGLFTVAAPAPGAP SPPATLAHLLPAPAMYSLLETELKNPVGTPTQAAGTGGPAAPGGAGKSSANAAGGANSGG GSSGGASGGGGGTDQDRVKRPMNAFMVWSRGQRRKMALENPKMHNSEISKRLGADWK LLTDAEKRPFIDEAKRLRAVHMKEYPDYKYRPRRKTKTLLKKDKYSLPSGLLPPGAAAAAAA AAAAAAAASSPVGVGQRLDTYTHVNGWANGAYSLVQEQLGYAQPPSMSSPPPPPALPPM HRYDMAGLQYSPMMPPGAQSYMNVAAAAAAASGYGGMAPSATAAAAAAYGQQPATAAA AAAAAAAMSLGPMGSVVKSEPSSPPPAIASHSQRACLGDLRDMISMYLPPGGDAADAASP LPGGRLHGVHQHYQGAGTAVNGTVPLTHI (SEQ ID NO:127; NP_005625), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:127.

In some embodiments, the nucleic acid sequence encoding SOX3 comprises the nucleic acid sequence: ATGCGACCTGTTCGAGAGAACTCATCAGGTGCGAGAAGCCCGCGGGTTCCTGCTGATT TGGCGCGGAGCATTTTGATAAGCCTACCCTTCCCGCCGGACTCGCTGGCCCACAGGC CCCCAAGCTCCGCTCCGACGGAGTCCCAGGGCCTTTTCACCGTGGCCGCTCCAGCCC CGGGAGCGCCTTCTCCTCCCGCCACGCTGGCGCACCTTCTTCCCGCCCCGGCAATGT ACAGCCTTCTGGAGACTGAACTCAAGAACCCCGTAGGGACACCCACACAAGCGGCGG GCACCGGCGGCCCCGCAGCCCCGGGAGGCGCAGGCAAGAGTAGTGCGAACGCAGCC GGCGGCGCGAACTCGGGCGGCGGCAGCAGCGGTGGTGCGAGCGGAGGTGGCGGGG GTACAGACCAGGACCGTGTGAAACGGCCCATGAACGCCTTCATGGTATGGTCCCGCG GGCAGCGGCGCAAAATGGCCCTGGAGAACCCCAAGATGCACAATTCTGAGATCAGCA AGCGCTTGGGCGCCGACTGGAAACTGCTGACCGACGCCGAGAAGCGACCATTCATCG ACGAGGCCAAGCGACTTCGCGCCGTGCACATGAAGGAGTATCCGGACTACAAGTACC GACCGCGCCGCAAGACCAAGACGCTGCTCAAGAAAGATAAGTACTCCCTGCCCAGCG GCCTCCTGCCTCCCGGTGCCGCGGCCGCCGCCGCCGCTGCCGCGGCCGCAGCCGCT GCCGCCAGCAGTCCGGTGGGCGTGGGCCAGCGCCTGGACACGTACACGCACGTGAA CGGCTGGGCCAACGGCGCGTACTCGCTGGTGCAGGAGCAGCTGGGCTACGCGCAGC CCCCGAGCATGAGCAGCCCGCCGCCGCCGCCCGCGCTGCCGCCGATGCACCGCTAC GACATGGCCGGCCTGCAGTACAGCCCAATGATGCCGCCCGGCGCTCAGAGCTACATG AACGTCGCTGCCGCGGCCGCCGCCGCCTCGGGCTACGGGGGCATGGCGCCCTCAGC CACAGCAGCCGCGGCCGCCGCCTACGGGCAGCAGCCCGCCACCGCCGCGGCCGCA GCTGCGGCCGCAGCCGCCATGAGCCTGGGCCCCATGGGCTCGGTAGTGAAGTCTGA GCCCAGCTCGCCGCCGCCCGCCATCGCATCGCACTCTCAGCGCGCGTGCCTCGGCG ACCTGCGCGACATGATCAGCATGTACCTGCCACCCGGCGGGGACGCGGCCGACGCC GCCTCTCCGCTGCCCGGCGGTCGCCTGCACGGCGTGCACCAGCACTACCAGGGCGC CGGGACTGCAGTCAACGGAACGGTGCCGCTGACCCACATC (SEQ ID NO:128; NM_005634), or a nucleic acid sequence that hybridizes to a nucleic acid sequence consisting of SEQ ID NO:128 under stringent hybridization conditions.

In some embodiments, SRY box 14 (SOX14) comprises the amino acid sequence: MSKPSDHIKRPMNAFMVWSRGQRRKMAQENPKMHNSEISKRLGAEWKLLSEAEKRPYID EAKRLRAQHMKEHPDYKYRPRRKPKNLLKKDRYVFPLPYLGDTDPLKAAGLPVGASDGLL SAPEKARAFLPPASAPYSLLDPAQFSSSAIQKMGEVPHTLATGALPYASTLGYQNGAFGSL SCPSQHTHTHPSPTNPGYVVPCNCTAWSASTLQPPVAYILFPGMTKTGIDPYSSAHATAM (SEQ ID NO:129; NP_004180), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:129.

In some embodiments, the nucleic acid sequence encoding SOX14 comprises the nucleic acid sequence: ATGTCCAAACCTTCAGACCACATCAAGCGGCCCATGAACGCCTTCATGGTATGGTCCC GGGGCCAGCGGCGCAAGATGGCCCAGGAAAACCCCAAGATGCACAACTCGGAGATCA GCAAACGCCTAGGTGCCGAATGGAAGCTTCTGTCCGAGGCAGAGAAGCGGCCATACA TCGATGAAGCCAAGCGGCTACGCGCCCAGCACATGAAGGAGCACCCTGACTACAAGT ACCGACCTCGGCGCAAGCCCAAGAACCTGCTCAAGAAGGACAGGTATGTCTTCCCCTT GCCCTACCTGGGCGACACGGACCCGCTCAAGGCGGCTGGCCTGCCCGTGGGGGCCT CCGACGGCCTCCTGAGCGCGCCCGAGAAAGCCCGGGCCTTCTTGCCGCCGGCCTCG GCGCCCTACTCCCTGCTGGACCCCGCGCAGTTTAGCTCGAGCGCCATCCAGAAGATG GGCGAAGTGCCCCACACCTTGGCTACCGGCGCTCTGCCCTACGCGTCCACCCTGGGC TACCAGAACGGCGCCTTCGGCAGCCTCAGCTGCCCCAGCCAGCACACGCACACGCAC CCGTCCCCCACCAACCCTGGCTACGTGGTGCCCTGTAACTGTACCGCCTGGTCTGCCT CCACCCTGCAGCCCCCCGTCGCCTACATCCTCTTCCCAGGCATGACCAAGACTGGCAT AGACCCTTATTCGTCAGCCCACGCTACGGCCATG (SEQ ID NO:130; NM_004189), or a nucleic acid sequence that hybridizes to a nucleic acid sequence consisting of SEQ ID NO:130 under stringent hybridization conditions.

In some embodiments, SRY-box 21 (SOX21) comprises the amino acid sequence: MSKPVDHVKRPMNAFMVWSRAQRRKMAQENPKMHNSEISKRLGAEWKLLTESEKRPFID EAKRLRAMHMKEHPDYKYRPRRKPKTLLKKDKFAFPVPYGLGGVADAEHPALKAGAGLHA GAGGGLVPESLLANPEKAAAAAAAAAARVFFPQSAAAAAAAAAAAAAGSPYSLLDLGSKM AEISSSSSGLPYASSLGYPTAGAGAFHGAAAAAAAAAAAAGGHTHSHPSPGNPGYMIPCN CSAWPSPGLQPPLAYILLPGMGKPQLDPYPAAYAAAL (SEQ ID NO:131; NP_009015), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:131.

In some embodiments, the nucleic acid sequence encoding SOX21 comprises the nucleic acid sequence: ATGTCCAAGCCGGTGGACCACGTCAAGCGGCCCATGAACGCCTTCATGGTGTGGTCG CGGGCTCAGCGGCGCAAGATGGCCCAGGAGAACCCCAAGATGCACAACTCGGAGATC AGCAAGCGCTTGGGCGCCGAGTGGAAACTGCTCACAGAGTCGGAGAAGCGGCCGTTC ATCGACGAGGCCAAGCGTCTACGCGCCATGCACATGAAGGAGCACCCCGACTACAAG TACCGGCCGCGGCGCAAGCCCAAGACGCTGCTCAAGAAGGACAAGTTCGCCTTCCCG GTGCCCTACGGCCTGGGCGGCGTGGCGGACGCCGAGCACCCTGCGCTCAAGGCGGG CGCCGGGCTGCACGCGGGGGCGGGCGGCGGCCTGGTGCCTGAGTCGCTGCTCGCC AATCCCGAGAAGGCGGCCGCGGCCGCCGCCGCTGCCGCCGCACGCGTCTTCTTCCC GCAGTCGGCCGCTGCCGCCGCCGCTGCCGCCGCCGCCGCCGCCGCGGGCAGCCCC TACTCGCTGCTCGACCTGGGCTCCAAAATGGCAGAGATCTCGTCGTCCTCGTCCGGCC TCCCGTACGCGTCGTCGCTGGGCTACCCGACCGCGGGCGCGGGCGCCTTCCACGGC GCGGCGGCGGCGGCTGCAGCGGCGGCCGCCGCCGCCGGGGGGCACACGCACTCGC ACCCCAGCCCGGGCAACCCGGGCTACATGATCCCGTGCAACTGCAGCGCGTGGCCCA GCCCCGGGCTGCAGCCGCCGCTCGCCTACATCCTGCTGCCGGGCATGGGCAAGCCC CAGCTGGACCCCTACCCCGCGGCCTACGCTGCCGCGCTA (SEQ ID NO:132; NM_007084), or a nucleic acid sequence that hybridizes to a nucleic acid sequence consisting of SEQ ID NO:132 under stringent hybridization conditions.

In some embodiments, SRY-box 4 (SOX4) comprises the amino acid sequence: MVQQTNNAENTEALLAGESSDSGAGLELGIASSPTPGSTASTGGKADDPSWCKTPSGHIK RPMNAFMVWSQIERRKIMEQSPDMHNAEISKRLGKRWKLLKDSDKIPFIREAERLRLKHMA DYPDYKYRPRKKVKSGNANSSSSAAASSKPGEKGDKVGGSGGGGHGGGGGGGSSNAG GGGGGASGGGANSKPAQKKSCGSKVAGGAGGGVSKPHAKLILAGGGGGGKAAAAAAAS FAAEQAGAAALLPLGAAADHHSLYKARTPSASASASSAASASAALAAPGKHLAEKKVKRVY LFGGLGTSSSPVGGVGAGADPSDPLGLYEEEGAGCSPDAPSLSGRSSAASSPAAGRSPA DHRGYASLRAASPAPSSAPSHASSSASSHSSSSSSSGSSSSDDEFEDDLLDLNPSSNFES MSLGSFSSSSALDRDLDFNFEPGSGSHFEFPDYCTPEVSEMISGDWLESSISNLVFTY (SEQ ID NO:133; NP_003098), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:133.

In some embodiments, the nucleic acid sequence encoding SOX4 comprises the nucleic acid sequence: ATGGTGCAGCAAACCAACAATGCCGAGAACACGGAAGCGCTGCTGGCCGGCGAGAGC TCGGACTCGGGCGCCGGCCTCGAGCTGGGAATCGCCTCCTCCCCCACGCCCGGCTC CACCGCCTCCACGGGCGGCAAGGCCGACGACCCGAGCTGGTGCAAGACCCCGAGTG GGCACATCAAGCGACCCATGAACGCCTTCATGGTGTGGTCGCAGATCGAGCGGCGCA AGATCATGGAGCAGTCGCCCGACATGCACAACGCCGAGATCTCCAAGCGGCTGGGCA AACGCTGGAAGCTGCTCAAAGACAGCGACAAGATCCCTTTCATTCGAGAGGCGGAGCG GCTGCGCCTCAAGCACATGGCTGACTACCCCGACTACAAGTACCGGCCCAGGAAGAA GGTGAAGTCCGGCAACGCCAACTCCAGCTCCTCGGCCGCCGCCTCCTCCAAGCCGGG GGAGAAGGGAGACAAGGTCGGTGGCAGTGGCGGGGGCGGCCATGGGGGCGGCGGC GGCGGCGGGAGCAGCAACGCGGGGGGAGGAGGCGGCGGTGCGAGTGGCGGCGGC GCCAACTCCAAACCGGCGCAGAAAAAGAGCTGCGGCTCCAAAGTGGCGGGCGGCGC GGGCGGTGGGGTTAGCAAACCGCACGCCAAGCTCATCCTGGCAGGCGGCGGCGGCG GCGGGAAAGCAGCGGCTGCCGCCGCCGCCTCCTTCGCCGCCGAACAGGCGGGGGCC GCCGCCCTGCTGCCCCTGGGCGCCGCCGCCGACCACCACTCGCTGTACAAGGCGCG GACTCCCAGCGCCTCGGCCTCCGCCTCCTCGGCAGCCTCGGCCTCCGCAGCGCTCGC GGCCCCGGGCAAGCACCTGGCGGAGAAGAAGGTGAAGCGCGTCTACCTGTTCGGCG GCCTGGGCACGTCGTCGTCGCCCGTGGGCGGCGTGGGCGCGGGAGCCGACCCCAG CGACCCCCTGGGCCTGTACGAGGAGGAGGGCGCGGGCTGCTCGCCCGACGCGCCCA GCCTGAGCGGCCGCAGCAGCGCCGCCTCGTCCCCCGCCGCCGGCCGCTCGCCCGCC GACCACCGCGGCTACGCCAGCCTGCGCGCCGCCTCGCCCGCCCCGTCCAGCGCGCC CTCGCACGCGTCCTCCTCGGCCTCGTCCCACTCCTCCTCTTCCTCCTCCTCGGGCTCC TCGTCCTCCGACGACGAGTTCGAAGACGACCTGCTCGACCTGAACCCCAGCTCAAACT TTGAGAGCATGTCCCTGGGCAGCTTCAGTTCGTCGTCGGCGCTCGACCGGGACCTGG ATTTTAACTTCGAGCCCGGCTCCGGCTCGCACTTCGAGTTCCCGGACTACTGCACGCC CGAGGTGAGCGAGATGATCTCGGGAGACTGGCTCGAGTCCAGCATCTCCAACCTGGT TTTCACCTAC (SEQ ID NO:134; NM_003107), or a nucleic acid sequence that hybridizes to a nucleic acid sequence consisting of SEQ ID NO:134 under stringent hybridization conditions.

In some embodiments, SRY-box 11 (SOX11) comprises the amino acid sequence: MVQQAESLEAESNLPREALDTEEGEFMACSPVALDESDPDWCKTASGHIKRPMNAFMVW SKIERRKIMEQSPDMHNAEISKRLGKRWKMLKDSEKIPFIREAERLRLKHMADYPDYKYRP RKKPKMDPSAKPSASQSPEKSAAGGGGGSAGGGAGGAKTSKGSSKKCGKLKAPAAAGA KAGAGKAAQSGDYGGAGDDYVLGSLRVSGSGGGGAGKTVKCVFLDEDDDDDDDDDELQ LQIKQEPDEEDEEPPHQQLLQPPGQQPSQLLRRYNVAKVPASPTLSSSAESPEGASLYDE VRAGATSGAGGGSRLYYSFKNITKQHPPPLAQPALSPASSRSVSTSSSSSSGSSSGSSGE DADDLMFDLSLNFSQSAHSASEQQLGGGAAAGNLSLSLVDKDLDSFSEGSLGSHFEFPDY CTPELSEMIAGDWLEANFSDLVFTY (SEQ ID NO:135; NP_003099), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:135.

In some embodiments, the nucleic acid sequence encoding SOX11 comprises the nucleic acid sequence: ATGGTGCAGCAGGCGGAGAGCTTGGAAGCGGAGAGCAACCTGCCCCGGGAGGCGCT GGACACGGAGGAGGGCGAATTCATGGCTTGCAGCCCGGTGGCCCTGGACGAGAGCG ACCCAGACTGGTGCAAGACGGCGTCGGGCCACATCAAGCGGCCGATGAACGCGTTCA TGGTATGGTCCAAGATCGAACGCAGGAAGATCATGGAGCAGTCTCCGGACATGCACAA CGCCGAGATCTCCAAGAGGCTGGGCAAGCGCTGGAAAATGCTGAAGGACAGCGAGAA GATCCCGTTCATCCGGGAGGCGGAGCGGCTGCGGCTCAAGCACATGGCCGACTACCC CGACTACAAGTACCGGCCCCGGAAAAAGCCCAAAATGGACCCCTCGGCCAAGCCCAG CGCCAGCCAGAGCCCAGAGAAGAGCGCGGCCGGCGGCGGCGGCGGGAGCGCGGGC GGAGGCGCGGGCGGTGCCAAGACCTCCAAGGGCTCCAGCAAGAAATGCGGCAAGCT CAAGGCCCCCGCGGCCGCGGGCGCCAAGGCGGGCGCGGGCAAGGCGGCCCAGTCC GGGGACTACGGGGGCGCGGGCGACGACTACGTGCTGGGCAGCCTGCGCGTGAGCG GCTCGGGCGGCGGCGGCGCGGGCAAGACGGTCAAGTGCGTGTTTCTGGATGAGGAC GACGACGACGACGACGACGACGACGAGCTGCAGCTGCAGATCAAACAGGAGCCGGAC GAGGAGGACGAGGAACCACCGCACCAGCAGCTCCTGCAGCCGCCGGGGCAGCAGCC GTCGCAGCTGCTGAGACGCTACAACGTCGCCAAAGTGCCCGCCAGCCCTACGCTGAG CAGCTCGGCGGAGTCCCCCGAGGGAGCGAGCCTCTACGACGAGGTGCGGGCCGGCG CGACCTCGGGCGCCGGGGGCGGCAGCCGCCTCTACTACAGCTTCAAGAACATCACCA AGCAGCACCCGCCGCCGCTCGCGCAGCCCGCGCTGTCGCCCGCGTCCTCGCGCTCG GTGTCCACCTCCTCGTCCAGCAGCAGCGGCAGCAGCAGCGGCAGCAGCGGCGAGGA CGCCGACGACCTGATGTTCGACCTGAGCTTGAATTTCTCTCAAAGCGCGCACAGCGCC AGCGAGCAGCAGCTGGGGGGCGGCGCGGCGGCCGGGAACCTGTCCCTGTCGCTGGT GGATAAGGATTTGGATTCGTTCAGCGAGGGCAGCCTGGGCTCCCACTTCGAGTTCCCC GACTACTGCACGCCGGAGCTGAGCGAGATGATCGCGGGGGACTGGCTGGAGGCGAA CTTCTCCGACCTGGTGTTCACATAT (SEQ ID NO:136; NM_003108), or a nucleic acid sequence that hybridizes to a nucleic acid sequence consisting of SEQ ID NO:136 under stringent hybridization conditions.

In some embodiments, SRY-box 12 (SOX12) comprises the amino acid sequence: MVQQRGARAKRDGGPPPPGPGPAEEGAREPGWCKTPSGHIKRPMNAFMVWSQHERRKI MDQWPDMHNAEISKRLGRRWQLLQDSEKIPFVREAERLRLKHMADYPDYKYRPRKKSKG APAKARPRPPGGSGGGSRLKPGPQLPGRGGRRAAGGPLGGGAAAPEDDDEDDDEELLE VRLVETPGRELWRMVPAGRAARGQAERAQGPSGEGAAAAAAASPTPSEDEEPEEEEEEA AAAEEGEEETVASGEESLGFLSRLPPGPAGLDCSALDRDPDLQPPSGTSHFEFPDYCTPE VTEMIAGDWRPSSIADLVFTY (SEQ ID NO:137; NP_008874), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:137.

In some embodiments, the nucleic acid sequence encoding SOX12 comprises the nucleic acid sequence: ATGGTGCAGCAGCGGGGCGCGAGGGCCAAGCGGGACGGCGGGCCGCCGCCCCCGG GACCCGGGCCGGCCGAGGAGGGGGCGCGCGAGCCCGGCTGGTGCAAGACCCCGAG CGGCCACATCAAGAGGCCGATGAACGCATTCATGGTGTGGTCGCAGCACGAACGGCG GAAGATCATGGACCAGTGGCCCGACATGCACAACGCCGAGATCTCCAAGCGCCTGGG CCGCCGCTGGCAGCTGCTGCAGGACTCGGAGAAGATCCCGTTCGTGCGGGAGGCGG AGCGGCTGCGGCTCAAGCACATGGCGGATTACCCGGACTACAAGTACCGGCCGCGCA AAAAGAGCAAGGGGGCGCCCGCCAAGGCGCGGCCCCGCCCCCCCGGTGGTAGCGGT GGCGGCAGCCGGCTCAAGCCCGGGCCGCAGCTGCCTGGCCGCGGGGGCCGCCGAG CAGCGGGAGGGCCTTTGGGGGGCGGGGCGGCGGCGCCCGAGGACGACGATGAAGA CGACGACGAGGAGCTGCTGGAAGTGCGCCTGGTCGAGACCCCGGGGCGGGAGCTGT GGAGGATGGTCCCGGCGGGACGGGCCGCTCGGGGACAAGCGGAGCGCGCCCAAGG GCCGTCGGGCGAGGGGGCGGCCGCCGCCGCCGCCGCCTCCCCGACACCGTCGGAG GACGAGGAGCCGGAGGAAGAGGAGGAGGAGGCGGCAGCGGCTGAGGAAGGTGAAG AGGAGACGGTGGCGTCGGGGGAGGAGTCGCTGGGCTTTCTGTCCAGGCTGCCCCCT GGCCCGGCCGGCCTGGACTGCAGCGCCCTGGATCGCGACCCGGACCTGCAGCCTCC CTCGGGCACGTCGCACTTCGAGTTCCCGGACTACTGCACCCCCGAGGTTACCGAGAT GATCGCGGGGGACTGGCGCCCGTCTAGCATCGCAGACCTGGTTTTCACCTAC (SEQ ID NO:138; NM_006943), or a nucleic acid sequence that hybridizes to a nucleic acid sequence consisting of SEQ ID NO:138 under stringent hybridization conditions.

In some embodiments, SRY-box 5 (SOX5) comprises the amino acid sequence: MLTDPDLPQEFERMSSKRPASPYGEADGEVAMVTSRQKVEEEESDGLPAFHLPLHVSFPN KPHSEEFQPVSLLTQETCGHRTPTSQHNTMEVDGNKVMSSFAPHNSSTSPQKAEEGGRQ SGESLSSTALGTPERRKGSLADVVDTLKQRKMEELIKNEPEETPSIEKLLSKDWKDKLLAM GSGNFGEIKGTPESLAEKERQLMGMINQLTSLREQLLAAHDEQKKLAASQIEKQRQQMELA KQQQEQIARQQQQLLQQQHKINLLQQQIQVQGQLPPLMIPVFPPDQRTLAAAAQQGFLLPP GFSYKAGCSDPYPVQLIPTTMAAAAAATPGLGPLQLQQLYAAQLAAMQVSPGGKLPGIPQ GNLGAAVSPTSIHTDKSTNSPPPKSKDEVAQPLNLSAKPKTSDGKSPTSPTSPHMPALRIN SGAGPLKASVPAALASPSARVSTIGYLNDHDAVTKAIQEARQMKEQLRREQQVLDGKVAVV NSLGLNNCRTEKEKTTLESLTQQLAVKQNEEGKFSHAMMDFNLSGDSDGSAGVSESRIYR ESRGRGSNEPHIKRPMNAFMVWAKDERRKILQAFPDMHNSNISKILGSRWKAMTNLEKQP YYEEQARLSKQHLEKYPDYKYKPRPKRTCLVDGKKLRIGEYKAIMRNRRQEMRQYFNVGQ QAQIPIATAGVVYPGAIAMAGMPSPHLPSEHSSVSSSPEPGMPVIQSTYGVKGEEPHIKEEI QAEDINGEIYDEYDEEEDDPDVDYGSDSENHIAGQAN (SEQ ID NO:139; NP_008871), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:139.

In some embodiments, the nucleic acid sequence encoding SOX5 comprises the nucleic acid sequence: ATGCTTACTGACCCTGATTTACCTCAGGAGTTTGAAAGGATGTCTTCCAAGCGACCAGC CTCTCCGTATGGGGAAGCAGATGGAGAGGTAGCCATGGTGACAAGCAGACAGAAAGT GGAAGAAGAGGAGAGTGACGGGCTCCCAGCCTTTCACCTTCCCTTGCATGTGAGTTTT CCCAACAAGCCTCACTCTGAGGAATTTCAGCCAGTTTCTCTGCTGACGCAAGAGACTTG TGGCCATAGGACTCCCACTTCTCAGCACAATACAATGGAAGTTGATGGCAATAAAGTTA TGTCTTCATTTGCCCCACACAACTCATCTACCTCACCTCAGAAGGCAGAAGAAGGTGG GCGACAGAGTGGCGAGTCCTTGTCTAGTACAGCCCTGGGAACTCCTGAACGGCGCAA GGGCAGTTTAGCTGATGTTGTTGACACCTTGAAGCAGAGGAAAATGGAAGAGCTCATC AAAAACGAGCCGGAAGAAACCCCCAGTATTGAAAAACTACTCTCAAAGGACTGGAAAG ACAAGCTTCTTGCAATGGGATCGGGGAACTTTGGCGAAATAAAAGGGACTCCCGAGAG CTTAGCTGAGAAAGAAAGGCAACTCATGGGTATGATCAACCAGCTGACCAGCCTCCGA GAGCAGCTGTTGGCTGCCCACGATGAGCAGAAGAAACTAGCTGCCTCTCAGATTGAGA AACAGCGTCAGCAAATGGAGCTGGCCAAGCAGCAACAAGAACAAATTGCAAGACAGCA GCAGCAGCTTCTACAGCAACAACACAAAATCAATTTGCTCCAGCAACAGATCCAGGTTC AAGGTCAGCTGCCGCCATTAATGATTCCCGTATTCCCTCCTGATCAACGGACACTGGCT GCAGCTGCCCAGCAAGGATTCCTCCTCCCTCCAGGCTTCAGCTATAAGGCTGGATGTA GTGACCCTTACCCTGTTCAGCTGATCCCAACTACCATGGCAGCTGCTGCCGCAGCAAC ACCAGGCTTAGGCCCACTCCAACTGCAGCAGTTATATGCTGCCCAGCTAGCTGCAATG CAGGTATCTCCAGGAGGGAAGCTGCCAGGCATACCCCAAGGCAACCTTGGTGCTGCT GTATCTCCTACCAGCATTCACACAGACAAGAGCACAAACAGCCCACCACCCAAAAGCA AGGATGAAGTGGCACAGCCACTGAACCTATCAGCTAAACCCAAGACCTCTGATGGCAA ATCACCCACATCACCCACCTCTCCCCATATGCCAGCTCTGAGAATAAACAGTGGGGCA GGCCCCCTCAAAGCCTCTGTCCCAGCAGCGTTAGCTAGTCCTTCAGCCAGAGTTAGCA CAATAGGTTACTTAAATGACCATGATGCTGTCACCAAGGCAATCCAAGAAGCTCGGCAA ATGAAGGAGCAACTCCGACGGGAACAACAGGTGCTTGATGGGAAGGTGGCTGTTGTG AATAGTCTGGGTCTCAATAACTGCCGAACAGAAAAGGAAAAAACAACACTGGAGAGTCT GACTCAGCAACTGGCAGTTAAACAGAATGAAGAAGGAAAATTTAGCCATGCAATGATGG ATTTCAATCTGAGTGGAGATTCTGATGGAAGTGCTGGAGTCTCAGAGTCAAGAATTTAT AGGGAATCCCGAGGGCGTGGTAGCAATGAACCCCACATAAAGCGTCCAATGAATGCCT TCATGGTGTGGGCTAAAGATGAACGGAGAAAGATCCTTCAAGCCTTTCCTGACATGCA CAACTCCAACATCAGCAAGATATTGGGATCTCGCTGGAAAGCTATGACAAACCTAGAGA AACAGCCATATTATGAGGAGCAAGCCCGTCTCAGCAAGCAGCACCTGGAGAAGTACCC TGACTATAAGTACAAGCCCAGGCCAAAGCGCACCTGCCTGGTGGATGGCAAAAAGCTG CGCATTGGTGAATACAAGGCAATCATGCGCAACAGGCGGCAGGAAATGCGGCAGTACT TCAATGTTGGGCAACAAGCACAGATCCCCATTGCCACTGCTGGTGTTGTGTACCCTGG AGCCATCGCCATGGCTGGGATGCCCTCCCCTCACCTGCCCTCGGAGCACTCAAGCGT GTCTAGCAGCCCAGAGCCTGGGATGCCTGTTATCCAGAGCACTTACGGTGTGAAAGGA GAGGAGCCACATATCAAAGAAGAGATACAGGCCGAGGACATCAATGGAGAAATTTATG ATGAGTACGACGAGGAAGAGGATGATCCAGATGTAGATTATGGGAGTGACAGTGAAAA CCATATTGCAGGACAAGCCAAC (SEQ ID NO:140; NM_006940), or a nucleic acid sequence that hybridizes to a nucleic acid sequence consisting of SEQ ID NO:140 under stringent hybridization conditions.

In some embodiments, SRY-box 6 (SOX6) comprises the amino acid sequence: MGRMSSKQATSPFACAADGEDAMTQDLTSREKEEGSDQHVASHLPLHPIMHNKPHSEEL PTLVSTIQQDADWDSVLSSQQRMESENNKLCSLYSFRNTSTSPHKPDEGSRDREIMTSVT FGTPERRKGSLADVVDTLKQKKLEEMTRTEQEDSSCMEKLLSKDWKEKMERLNTSELLGE IKGTPESLAEKERQLSTMITQLISLREQLLAAHDEQKKLAASQIEKQRQQMDLARQQQEQIA RQQQQLLQQQHKINLLQQQIQVQGHMPPLMIPIFPHDQRTLAAAAAAQQGFLFPPGITYKP GDNYPVQFIPSTMAAAAASGLSPLQLQQLYAAQLASMQVSPGAKMPSTPQPPNTAGTVSP TGIKNEKRGTSPVTQVKDEAAAQPLNLSSRPKTAEPVKSPTSPTQNLFPASKTSPVNLPNK SSIPSPIGGSLGRGSSLGKWKSQHQEETYELDILSSLNSPALFGDQDTVMKAIQEARKMRE QIQREQQQQQPHGVDGKLSSINNMGLNSCRNEKERTRFENLGPQLTGKSNEDGKLGPGVI DLTRPEDAEGSKAMNGSAAKLQQYYCWPTGGATVAEARVYRDARGRASSEPHIKRPMNA FMVWAKDERRKILQAFPDMHNSNISKILGSRWKSMSNQEKQPYYEEQARLSKIHLEKYPNY KYKPRPKRTCIVDGKKLRIGEYKQLMRSRRQEMRQFFTVGQQPQIPITTGTGVVYPGAITM ATTTPSPQMTSDCSSTSASPEPSLPVIQSTYGMKTDGGSLAGNEMINGEDEMEMYDDYED DPKSDYSSENEAPEAVSAN (SEQ ID NO:141; NP_059978), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:141.

In some embodiments, the nucleic acid sequence encoding SOX6 comprises the nucleic acid sequence: ATGGGAAGAATGTCTTCCAAGCAAGCCACCTCTCCATTTGCCTGTGCAGCTGATGGAG AGGATGCAATGACCCAGGATTTAACCTCAAGGGAAAAGGAAGAGGGCAGTGATCAACA TGTGGCCTCCCATCTGCCTCTGCACCCCATAATGCACAACAAACCTCACTCTGAGGAG CTACCAACACTTGTCAGTACCATTCAACAAGATGCTGACTGGGACAGCGTTCTGTCATC TCAGCAAAGAATGGAATCAGAGAATAATAAGTTATGTTCCCTATATTCCTTCCGAAATAC CTCTACCTCACCACATAAGCCTGACGAAGGGAGTCGGGACCGTGAGATAATGACCAGT GTTACTTTTGGAACCCCAGAGCGCCGCAAAGGGAGTCTTGCCGATGTGGTGGACACAC TGAAACAGAAGAAGCTTGAGGAAATGACTCGGACTGAACAAGAGGATTCCTCCTGCAT GGAAAAACTACTTTCAAAAGATTGGAAGGAAAAAATGGAAAGACTAAATACCAGTGAAC TTCTTGGAGAAATTAAAGGTACACCTGAGAGCCTGGCAGAAAAAGAACGGCAGCTCTC CACCATGATTACCCAGCTGATCAGTTTACGGGAGCAGCTACTGGCAGCGCATGATGAA CAGAAAAAACTGGCAGCGTCACAAATTGAGAAACAACGGCAGCAAATGGACCTTGCTC GCCAACAGCAAGAACAGATTGCGAGACAACAGCAGCAACTTCTGCAACAGCAGCACAA AATTAATCTCCTGCAGCAACAGATCCAGGTTCAGGGTCACATGCCTCCGCTCATGATCC CAATTTTTCCACATGACCAGCGGACTCTGGCAGCAGCTGCTGCTGCCCAACAGGGATT CCTCTTCCCCCCTGGAATAACATACAAACCAGGTGATAACTACCCCGTACAGTTCATTC CATCAACAATGGCAGCTGCTGCTGCTTCTGGACTCAGCCCTTTACAGCTCCAGCAGCT CTATGCCGCTCAGCTGGCCAGCATGCAGGTGTCACCTGGAGCAAAGATGCCATCAACT CCACAGCCACCAAACACAGCAGGGACGGTCTCACCTACTGGGATAAAAAATGAAAAGA GAGGGACCAGCCCTGTAACTCAAGTTAAGGATGAAGCAGCAGCACAGCCTCTGAATCT CTCATCCCGACCCAAGACAGCAGAGCCTGTAAAGTCCCCAACGTCTCCCACCCAGAAC CTCTTCCCAGCCAGCAAAACCAGCCCTGTCAATCTGCCAAACAAAAGCAGCATCCCTA GCCCCATTGGAGGAAGCCTGGGAAGAGGATCCTCTTTAGGTAAATGGAAAAGTCAACA CCAGGAAGAGACTTACGAATTAGATATCCTATCTAGTCTCAACTCCCCTGCCCTTTTTG GGGATCAGGATACAGTGATGAAAGCCATTCAGGAGGCGCGGAAGATGCGAGAGCAGA TCCAGCGGGAGCAACAGCAGCAACAGCCACATGGTGTTGACGGGAAACTGTCCTCCAT AAATAATATGGGGCTGAACAGCTGCAGGAATGAAAAGGAAAGAACGCGCTTTGAGAAT TTGGGGCCCCAGTTAACGGGAAAGTCAAATGAAGATGGAAAACTGGGCCCAGGTGTCA TCGACCTTACTCGGCCAGAAGATGCAGAGGGAAGTAAAGCAATGAATGGCTCTGCAGC TAAACTACAGCAGTATTATTGTTGGCCAACAGGAGGTGCCACTGTGGCTGAAGCACGA GTCTACAGGGACGCCCGCGGCCGTGCCAGCAGCGAGCCACACATTAAGCGACCAATG AATGCATTCATGGTTTGGGCAAAGGATGAGAGGAGAAAAATCCTTCAGGCCTTCCCCG ACATGCATAACTCCAACATTAGCAAAATCTTAGGATCTCGCTGGAAATCAATGTCCAAC CAGGAGAAGCAACCTTATTATGAAGAGCAGGCCCGGCTAAGCAAGATCCACTTAGAGA AGTACCCAAACTATAAATACAAACCCCGACCGAAACGCACCTGCATTGTTGATGGCAAA AAGCTTCGGATTGGGGAGTATAAGCAACTGATGAGGTCTCGGAGACAGGAGATGAGG CAGTTCTTTACTGTGGGGCAACAGCCTCAGATTCCAATCACCACAGGAACAGGTGTTGT GTATCCTGGTGCTATCACTATGGCAACTACCACACCATCGCCTCAGATGACATCTGACT GCTCTAGCACCTCGGCCAGCCCGGAGCCCAGCCTCCCGGTCATCCAGAGCACTTATG GTATGAAGACAGATGGCGGAAGCCTAGCTGGAAATGAAATGATCAATGGAGAGGATGA AATGGAAATGTATGATGACTATGAAGATGACCCCAAATCAGACTATAGCAGTGAAAATG AAGCCCCGGAGGCTGTCAGTGCCAAC (SEQ ID NO:142; NM_017508), or a nucleic acid sequence that hybridizes to a nucleic acid sequence consisting of SEQ ID NO:142 under stringent hybridization conditions.

In some embodiments, SRY-box 13 (SOX13) comprises the amino acid sequence: MSMRSPISAQLALDGVGTMVNCTIKSEEKKEPCHEAPQGSATAAEPQPGDPARASQDSAD PQAPAQGNFRGSWDCSSPEGNGSPEPKRPGVSEAASGSQEKLDFNRNLKEVVPAIEKLLS SDWKERFLGRNSMEAKDVKGTQESLAEKELQLLVMIHQLSTLRDQLLTAHSEQKNMAAML FEKQQQQMELARQQQEQIAKQQQQLIQQQHKINLLQQQIQQVNMPYVMIPAFPPSHQPLP VTPDSQLALPIQPIPCKPVEYPLQLLHSPPAPVVKRPGAMATHHPLQEPSQPLNLTAKPKAP ELPNTSSSPSLKMSSCVPRPPSHGGPTRDLQSSPPSLPLGFLGEGDAVTKAIQDARQLLHS HSGALDGSPNTPFRKDLISLDSSPAKERLEDGCVHPLEEAMLSCDMDGSRHFPESRNSSHI KRPMNAFMVWAKDERRKILQAFPDMHNSSISKILGSRWKSMTNQEKQPYYEEQARLSRQ HLEKYPDYKYKPRPKRTCIVEGKRLRVGEYKALMRTRRQDARQSYVIPPQAGQVQMSSSD VLYPRAAGMPLAQPLVEHYVPRSLDPNMPVIVNTCSLREEGEGTDDRHSVADGEMYRYSE DEDSEGEEKSDGELVVLTD (SEQ ID NO:143; NP_005677), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:143.

In some embodiments, the nucleic acid sequence encoding SOX13 comprises the nucleic acid sequence: ATGTCCATGAGGAGCCCCATCTCTGCCCAGCTGGCCCTGGATGGCGTTGGCACCATG GTGAACTGCACCATCAAGTCAGAGGAGAAGAAAGAGCCTTGCCACGAGGCCCCCCAG GGCTCAGCCACTGCCGCTGAACCTCAGCCTGGAGACCCAGCCCGGGCCTCCCAGGAT AGTGCTGACCCCCAAGCTCCAGCCCAGGGGAATTTCAGGGGCTCCTGGGACTGTAGC TCTCCAGAGGGTAATGGGTCCCCAGAACCCAAGAGACCAGGAGTGTCGGAGGCTGCC TCTGGAAGCCAGGAGAAGCTGGACTTCAACCGAAATTTGAAAGAAGTGGTGCCAGCCA TAGAGAAGCTGTTGTCCAGTGACTGGAAGGAGAGGTTTCTAGGAAGGAACTCTATGGA AGCCAAAGATGTCAAAGGGACCCAAGAGAGCCTAGCAGAGAAGGAGCTCCAGCTTCT GGTCATGATTCACCAGCTGTCCACCCTGCGGGACCAGCTCCTGACAGCCCACTCGGA GCAGAAGAACATGGCTGCCATGCTGTTTGAGAAGCAGCAGCAGCAGATGGAGCTTGC CCGGCAGCAGCAGGAGCAGATTGCAAAGCAGCAGCAGCAGCTGATTCAGCAGCAGCA TAAGATCAACCTCCTTCAGCAGCAGATCCAGCAGGTTAACATGCCTTATGTCATGATCC CAGCCTTCCCCCCAAGCCACCAACCTCTGCCTGTCACCCCTGACTCCCAGCTGGCCTT ACCCATTCAGCCCATTCCCTGCAAACCAGTGGAGTATCCGCTGCAGCTGCTGCACAGC CCCCCTGCCCCAGTGGTGAAGAGGCCTGGGGCCATGGCCACCCACCACCCCCTGCAG GAGCCCTCCCAGCCCCTGAACCTCACAGCCAAGCCCAAGGCCCCCGAGCTGCCCAAC ACCTCCAGCTCCCCAAGCCTGAAGATGAGCAGCTGTGTGCCCCGCCCCCCCAGCCAT GGAGGCCCCACGCGGGACCTGCAGTCCAGCCCCCCGAGCCTGCCTCTGGGCTTCCTT GGTGAAGGGGACGCTGTCACCAAAGCCATCCAGGATGCTCGGCAGCTGCTGCACAGC CACAGTGGGGCCTTGGATGGCTCCCCCAACACCCCCTTCCGTAAGGACCTCATCAGCC TGGACTCATCCCCAGCCAAGGAGCGGCTGGAGGACGGCTGTGTGCACCCACTGGAGG AAGCCATGCTGAGCTGCGACATGGATGGCTCCCGCCACTTCCCCGAGTCCCGAAACA GCAGCCACATCAAGAGGCCCATGAACGCCTTCATGGTGTGGGCCAAGGATGAGCGGA GGAAGATCCTGCAAGCCTTCCCAGACATGCACAACTCCAGCATCAGCAAGATCCTTGG ATCTCGCTGGAAGTCCATGACCAACCAGGAGAAGCAGCCCTACTATGAGGAACAGGCG CGGCTGAGCCGGCAGCACCTGGAGAAGTATCCTGACTACAAGTACAAGCCGCGGCCC AAGCGCACCTGCATCGTGGAGGGCAAGCGGCTGCGCGTGGGAGAGTACAAGGCCCT GATGAGGACCCGGCGTCAGGATGCCCGCCAGAGCTACGTGATCCCCCCGCAGGCTG GCCAGGTGCAGATGAGCTCCTCAGATGTCCTGTACCCTCGGGCAGCAGGCATGCCGC TGGCACAGCCACTGGTGGAGCACTATGTCCCTCGTAGCCTGGACCCCAACATGCCTGT GATCGTCAACACCTGCAGCCTCAGAGAGGAGGGTGAGGGCACAGATGACAGGCACTC GGTGGCTGATGGCGAGATGTACCGGTACAGCGAGGACGAGGACTCGGAGGGCGAAG AGAAGAGCGATGGGGAGTTGGTGGTGCTCACAGAC (SEQ ID NO:144; NM_005686), or a nucleic acid sequence that hybridizes to a nucleic acid sequence consisting of SEQ ID NO:144 under stringent hybridization conditions.

In some embodiments, SRY-box 8 (SOX8) comprises the amino acid sequence: MLDMSEARSQPPCSPSGTASSMSHVEDSDSDAPPSPAGSEGLGRAGVAVGGARGDPAE AADERFPACIRDAVSQVLKGYDWSLVPMPVRGGGGGALKAKPHVKRPMNAFMVWAQAA RRKLADQYPHLHNAELSKTLGKLWRLLSESEKRPFVEEAERLRVQHKKDHPDYKYQPRRR KSAKAGHSDSDSGAELGPHPGGGAVYKAEAGLGDGHHHGDHTGQTHGPPTPPTTPKTEL QQAGAKPELKLEGRRPVDSGRQNIDFSNVDISELSSEVMGTMDAFDVHEFDQYLPLGGPA PPEPGQAYGGAYFHAGASPVWAHKSAPSASASPTETGPPRPHIKTEQPSPGHYGDQPRG SPDYGSCSGQSSATPAAPAGPFAGSQGDYGDLQASSYYGAYPGYAPGLYQYPCFHSPRR PYASPLLNGLALPPAHSPTSHWDQPVYTTLTRP (SEQ ID NO:145; NP_055402), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:145.

In some embodiments, the nucleic acid sequence encoding SOX8 comprises the nucleic acid sequence ATGCTGGACATGAGCGAGGCCCGCTCCCAGCCGCCCTGCAGCCCGTCCGGCACCGC CAGCTCCATGTCGCACGTGGAGGACTCGGACTCGGACGCGCCGCCGTCTCCCGCCG GCTCCGAGGGCCTGGGCCGCGCGGGGGTCGCGGTGGGGGGCGCCCGGGGCGACCC GGCGGAGGCGGCGGACGAGCGCTTCCCGGCCTGCATCCGCGACGCCGTGTCGCAGG TGCTCAAGGGCTACGACTGGAGTCTGGTGCCCATGCCGGTGCGCGGCGGCGGCGGC GGCGCGCTCAAAGCCAAGCCGCATGTGAAGCGGCCCATGAACGCATTCATGGTGTGG GCGCAGGCGGCGCGCCGCAAGCTGGCCGACCAGTACCCGCACCTGCACAACGCCGA GCTCAGCAAGACGCTGGGCAAGCTGTGGCGCTTGCTGAGCGAGAGCGAGAAGCGGC CCTTCGTGGAGGAGGCAGAGCGCCTTCGCGTGCAGCACAAGAAGGACCACCCCGACT ACAAGTACCAGCCACGGCGCAGGAAGAGCGCCAAAGCCGGCCACAGCGACTCCGACT CGGGCGCGGAGCTGGGACCCCACCCTGGCGGCGGTGCCGTGTACAAGGCTGAAGCA GGGCTTGGAGATGGGCACCACCATGGCGACCACACAGGGCAGACCCACGGGCCGCC CACCCCGCCCACCACCCCCAAGACGGAGCTGCAGCAGGCGGGCGCCAAGCCGGAGC TGAAGCTGGAGGGACGCCGGCCGGTGGACAGCGGGCGCCAGAACATCGACTTCAGC AATGTGGACATCTCGGAGCTCAGCAGCGAGGTCATGGGCACCATGGACGCCTTCGAC GTCCACGAGTTCGACCAGTACCTGCCCCTGGGCGGCCCCGCCCCACCCGAGCCGGG CCAGGCCTATGGGGGCGCCTACTTCCACGCCGGGGCGTCCCCCGTGTGGGCCCACA AGAGTGCCCCGTCGGCCTCCGCGTCGCCCACCGAGACGGGTCCCCCACGGCCGCAC ATCAAGACGGAGCAGCCGAGCCCCGGCCACTACGGCGACCAGCCCCGAGGCTCGCC CGACTACGGTTCCTGCAGCGGCCAGTCCAGCGCCACCCCGGCCGCCCCCGCCGGCC CCTTCGCCGGCTCACAGGGCGACTATGGCGACCTGCAGGCCTCCAGCTACTATGGTG CCTACCCTGGCTACGCACCCGGCCTCTACCAGTACCCCTGCTTCCACTCGCCGCGCC GGCCCTACGCCTCACCCCTGCTCAACGGCCTGGCCCTGCCGCCCGCCCACAGCCCCA CCAGTCACTGGGACCAGCCGGTGTACACCACCCTGACCAGGCCC(SEQ ID NO:146; NM_014587), or a nucleic acid sequence that hybridizes to a nucleic acid sequence consisting of SEQ ID NO:146 under stringent hybridization conditions.

In some embodiments, SRY-box 9 (SOX9) comprises the amino acid sequence: MNLLDPFMKMTDEQEKGLSGAPSPTMSEDSAGSPCPSGSGSDTENTRPQENTFPKGEPD LKKESEEDKFPVCIREAVSQVLKGYDWTLVPMPVRVNGSSKNKPHVKRPMNAFMVWAQA ARRKLADQYPHLHNAELSKTLGKLWRLLNESEKRPFVEEAERLRVQHKKDHPDYKYQPRR RKSVKNGQAEAEEATEQTHISPNAIFKALQADSPHSSSGMSEVHSPGEHSGQSQGPPTPP TTPKTDVQPGKADLKREGRPLPEGGRQPPIDFRDVDIGELSSDVISNIETFDVNEFDQYLPP NGHPGVPATHGQVTYTGSYGISSTAATPASAGHVWMSKQQAPPPPPQQPPQAPPAPQAP PQPQAAPPQQPAAPPQQPQAHTLTTLSSEPGQSQRTHIKTEQLSPSHYSEQQQHSPQQIA YSPFNLPHYSPSYPPITRSQYDYTDHQNSSSYYSHAAGQGTGLYSTFTYMNPAQRPMYTPI ADTSGVPSIPQTHSPQHWEQPVYTQLTRP (SEQ ID NO:147; NP_000337), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:147.

In some embodiments, the nucleic acid sequence encoding SOX9 comprises the nucleic acid sequence: ATGAATCTCCTGGACCCCTTCATGAAGATGACCGACGAGCAGGAGAAGGGCCTGTCCG GCGCCCCCAGCCCCACCATGTCCGAGGACTCCGCGGGCTCGCCCTGCCCGTCGGGC TCCGGCTCGGACACCGAGAACACGCGGCCCCAGGAGAACACGTTCCCCAAGGGCGA GCCCGATCTGAAGAAGGAGAGCGAGGAGGACAAGTTCCCCGTGTGCATCCGCGAGGC GGTCAGCCAGGTGCTCAAAGGCTACGACTGGACGCTGGTGCCCATGCCGGTGCGCGT CAACGGCTCCAGCAAGAACAAGCCGCACGTCAAGCGGCCCATGAACGCCTTCATGGT GTGGGCGCAGGCGGCGCGCAGGAAGCTCGCGGACCAGTACCCGCACTTGCACAACG CCGAGCTCAGCAAGACGCTGGGCAAGCTCTGGAGACTTCTGAACGAGAGCGAGAAGC GGCCCTTCGTGGAGGAGGCGGAGCGGCTGCGCGTGCAGCACAAGAAGGACCACCCG GATTACAAGTACCAGCCGCGGCGGAGGAAGTCGGTGAAGAACGGGCAGGCGGAGGC AGAGGAGGCCACGGAGCAGACGCACATCTCCCCCAACGCCATCTTCAAGGCGCTGCA GGCCGACTCGCCACACTCCTCCTCCGGCATGAGCGAGGTGCACTCCCCCGGCGAGCA CTCGGGGCAATCCCAGGGCCCACCGACCCCACCCACCACCCCCAAAACCGACGTGCA GCCGGGCAAGGCTGACCTGAAGCGAGAGGGGCGCCCCTTGCCAGAGGGGGGCAGAC AGCCCCCTATCGACTTCCGCGACGTGGACATCGGCGAGCTGAGCAGCGACGTCATCT CCAACATCGAGACCTTCGATGTCAACGAGTTTGACCAGTACCTGCCGCCCAACGGCCA CCCGGGGGTGCCGGCCACGCACGGCCAGGTCACCTACACGGGCAGCTACGGCATCA GCAGCACCGCGGCCACCCCGGCGAGCGCGGGCCACGTGTGGATGTCCAAGCAGCAG GCGCCGCCGCCACCCCCGCAGCAGCCCCCACAGGCCCCGCCGGCCCCGCAGGCGC CCCCGCAGCCGCAGGCGGCGCCCCCACAGCAGCCGGCGGCACCCCCGCAGCAGCCA CAGGCGCACACGCTGACCACGCTGAGCAGCGAGCCGGGCCAGTCCCAGCGAACGCA CATCAAGACGGAGCAGCTGAGCCCCAGCCACTACAGCGAGCAGCAGCAGCACTCGCC CCAACAGATCGCCTACAGCCCCTTCAACCTCCCACACTACAGCCCCTCCTACCCGCCC ATCACCCGCTCACAGTACGACTACACCGACCACCAGAACTCCAGCTCCTACTACAGCC ACGCGGCAGGCCAGGGCACCGGCCTCTACTCCACCTTCACCTACATGAACCCCGCTC AGCGCCCCATGTACACCCCCATCGCCGACACCTCTGGGGTCCCTTCCATCCCGCAGA CCCACAGCCCCCAGCACTGGGAACAACCCGTCTACACACAGCTCACTCGACCT (SEQ ID NO:148; NM_000346), or a nucleic acid sequence that hybridizes to a nucleic acid sequence consisting of SEQ ID NO:148 under stringent hybridization conditions.

In some embodiments, SRY-box 10 (SOX10) comprises the amino acid sequence: MAEEQDLSEVELSPVGSEEPRCLSPGSAPSLGPDGGGGGSGLRASPGPGELGKVKKEQQ DGEADDDKFPVCIREAVSQVLSGYDWTLVPMPVRVNGASKSKPHVKRPMNAFMVWAQAA RRKLADQYPHLHNAELSKTLGKLWRLLNESDKRPFIEEAERLRMQHKKDHPDYKYQPRRR KNGKAAQGEAECPGGEAEQGGTAAIQAHYKSAHLDHRHPGEGSPMSDGNPEHPSGQSH GPPTPPTTPKTELQSGKADPKRDGRSMGEGGKPHIDFGNVDIGEISHEVMSNMETFDVAE LDQYLPPNGHPGHVSSYSAAGYGLGSALAVASGHSAWISKPPGVALPTVSPPGVDAKAQV KTETAGPQGPPHYTDQPSTSQIAYTSLSLPHYGSAFPSISRPQFDYSDHQPSGPYYGHSG QASGLYSAFSYMGPSQRPLYTAISDPSPSGPQSHSPTHWEQPVYTTLSRP (SEQ ID NO:149; NP_008872), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:149.

In some embodiments, the nucleic acid sequence encoding SOX10 comprises the nucleic acid sequence: ATGGCGGAGGAGCAGGACCTATCGGAGGTGGAGCTGAGCCCCGTGGGCTCGGAGGA GCCCCGCTGCCTGTCCCCGGGGAGCGCGCCCTCGCTAGGGCCCGACGGCGGCGGC GGCGGATCGGGCCTGCGAGCCAGCCCGGGGCCAGGCGAGCTGGGCAAGGTCAAGAA GGAGCAGCAGGACGGCGAGGCGGACGATGACAAGTTCCCCGTGTGCATCCGCGAGG CCGTCAGCCAGGTGCTCAGCGGCTACGACTGGACGCTGGTGCCCATGCCCGTGCGCG TCAACGGCGCCAGCAAAAGCAAGCCGCACGTCAAGCGGCCCATGAACGCCTTCATGG TGTGGGCTCAGGCAGCGCGCAGGAAGCTCGCGGACCAGTACCCGCACCTGCACAACG CTGAGCTCAGCAAGACGCTGGGCAAGCTCTGGAGGCTGCTGAACGAAAGTGACAAGC GCCCCTTCATCGAGGAGGCTGAGCGGCTCCGTATGCAGCACAAGAAAGACCACCCGG ACTACAAGTACCAGCCCAGGCGGCGGAAGAACGGGAAGGCCGCCCAGGGCGAGGCG GAGTGCCCCGGTGGGGAGGCCGAGCAAGGTGGGACCGCCGCCATCCAGGCCCACTA CAAGAGCGCCCACTTGGACCACCGGCACCCAGGAGAGGGCTCCCCCATGTCAGATGG TAACCCCGAGCACCCCTCAGGCCAGAGCCATGGCCCACCCACCCCTCCAACCACCCC GAAGACAGAGCTGCAGTCGGGCAAGGCAGACCCGAAGCGGGACGGGCGCTCCATGG GGGAGGGCGGGAAGCCTCACATCGACTTCGGCAACGTGGACATTGGTGAGATCAGCC ACGAGGTAATGTCCAACATGGAGACCTTTGATGTGGCTGAGTTGGACCAGTACCTGCC GCCCAATGGGCACCCAGGCCATGTGAGCAGCTACTCAGCAGCCGGCTATGGGCTGGG CAGTGCCCTGGCCGTGGCCAGTGGACACTCCGCCTGGATCTCCAAGCCACCAGGCGT GGCTCTGCCCACGGTCTCACCACCTGGTGTGGATGCCAAAGCCCAGGTGAAGACAGA GACCGCGGGGCCCCAGGGGCCCCCACACTACACCGACCAGCCATCCACCTCACAGAT CGCCTACACCTCCCTCAGCCTGCCCCACTATGGCTCAGCCTTCCCCTCCATCTCCCGC CCCCAGTTTGACTACTCTGACCATCAGCCCTCAGGACCCTATTATGGCCACTCGGGCC AGGCCTCTGGCCTCTACTCGGCCTTCTCCTATATGGGGCCCTCGCAGCGGCCCCTCTA CACGGCCATCTCTGACCCCAGCCCCTCAGGGCCCCAGTCCCACAGCCCCACACACTG GGAGCAGCCAGTATATACGACACTGTCCCGGCCC (SEQ ID NO:150; NM_006941), or a nucleic acid sequence that hybridizes to a nucleic acid sequence consisting of SEQ ID NO:150 under stringent hybridization conditions.

In some embodiments, SRY-box 7 (SOX7) comprises the amino acid sequence: MASLLGAYPWPEGLECPALDAELSDGQSPPAVPRPPGDKGSESRIRRPMNAFMVWAKDE RKRLAVQNPDLHNAELSKMLGKSWKALTLSQKRPYVDEAERLRLQHMQDYPNYKYRPRR KKQAKRLCKRVDPGFLLSSLSRDQNALPEKRSGSRGALGEKEDRGEYSPGTALPSLRGCY HEGPAGGGGGGTPSSVDTYPYGLPTPPEMSPLDVLEPEQTFFSSPCQEEHGHPRRIPHLP GHPYSPEYAPSPLHCSHPLGSLALGQSPGVSMMSPVPGCPPSPAYYSPATYHPLHSNLQA HLGQLSPPPEHPGFDALDQLSQVELLGDMDRNEFDQYLNTPGHPDSATGAMALSGHVPV SQVTPTGPTETSLISVLADATATYYNSYSVS (SEQ ID NO:151; NP_113627), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:151.

In some embodiments, the nucleic acid sequence encoding SOX7 comprises the nucleic acid sequence: ATGGCTTCGCTGCTGGGAGCCTACCCTTGGCCCGAGGGTCTCGAGTGCCCGGCCCTG GACGCCGAGCTGTCGGATGGACAATCGCCGCCGGCCGTCCCCCGGCCCCCGGGGGA CAAGGGCTCCGAGAGCCGTATCCGGCGGCCCATGAACGCCTTCATGGTTTGGGCCAA GGACGAGAGGAAACGGCTGGCAGTGCAGAACCCGGACCTGCACAACGCCGAGCTCA GCAAGATGCTGGGAAAGTCGTGGAAGGCGCTGACGCTGTCCCAGAAGAGGCCGTACG TGGACGAGGCGGAGCGGCTGCGCCTGCAGCACATGCAGGACTACCCCAACTACAAGT ACCGGCCGCGCAGGAAGAAGCAGGCCAAGCGGCTGTGCAAGCGCGTGGACCCGGGC TTCCTTCTGAGCTCCCTCTCCCGGGACCAGAACGCCCTGCCGGAGAAGAGAAGCGGC AGCCGGGGGGCGCTGGGGGAGAAGGAGGACAGGGGTGAGTACTCCCCCGGCACTGC CCTGCCCAGCCTCCGGGGCTGCTACCACGAGGGGCCGGCTGGTGGTGGCGGCGGCG GCACCCCGAGCAGTGTGGACACGTACCCGTACGGGCTGCCCACACCTCCTGAAATGT CTCCCCTGGACGTGCTGGAGCCGGAGCAGACCTTCTTCTCCTCCCCCTGCCAGGAGG AGCATGGCCATCCCCGCCGCATCCCCCACCTGCCAGGGCACCCGTACTCACCGGAGT ACGCCCCAAGCCCTCTCCACTGTAGCCACCCCCTGGGCTCCCTGGCCCTTGGCCAGT CCCCCGGCGTCTCCATGATGTCCCCTGTACCCGGCTGTCCCCCATCTCCTGCCTATTA CTCCCCGGCCACCTACCACCCACTCCACTCCAACCTCCAAGCCCACCTGGGCCAGCTT TCCCCGCCTCCTGAGCACCCTGGCTTCGACGCCCTGGATCAACTGAGCCAGGTGGAA CTCCTGGGGGACATGGATCGCAATGAATTCGACCAGTATTTGAACACTCCTGGCCACC CAGACTCCGCCACAGGGGCCATGGCCCTCAGTGGGCATGTTCCGGTCTCCCAGGTGA CACCAACGGGTCCCACAGAGACCAGCCTCATCTCCGTCCTGGCTGATGCCACGGCCA CGTACTACAACAGCTACAGTGTGTCA (SEQ ID NO:152; NM_031439), or a nucleic acid sequence that hybridizes to a nucleic acid sequence consisting of SEQ ID NO:152 under stringent hybridization conditions.

In some embodiments, SRY-box 17 (SOX17) comprises the amino acid sequence: MSSPDAGYASDDQSQTQSALPAVMAGLGPCPWAESLSPIGDMKVKGEAPANSGAPAGAA GRAKGESRIRRPMNAFMVWAKDERKRLAQQNPDLHNAELSKMLGKSWKALTLAEKRPFV EEAERLRVQHMQDHPNYKYRPRRRKQVKRLKRVEGGFLHGLAEPQAAALGPEGGRVAM DGLGLQFPEQGFPAGPPLLPPHMGGHYRDCQSLGAPPLDGYPLPTPDTSPLDGVDPDPA FFAAPMPGDCPAAGTYSYAQVSDYAGPPEPPAGPMHPRLGPEPAGPSIPGLLAPPSALHV YYGAMGSPGAGGGRGFQMQPQHQHQHQHQHHPPGPGQPSPPPEALPCRDGTDPSQPA ELLGEVDRTEFEQYLHFVCKPEMGLPYQGHDSGVNLPDSHGAISSVVSDASSAVYYCNYP DV (SEQ ID NO:153; NP_071899), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:153.

In some embodiments, the nucleic acid sequence encoding SOX17 comprises the nucleic acid sequence: ATGAGCAGCCCGGATGCGGGATACGCCAGTGACGACCAGAGCCAGACCCAGAGCGC GCTGCCCGCGGTGATGGCCGGGCTGGGCCCCTGCCCCTGGGCCGAGTCGCTGAGCC CCATCGGGGACATGAAGGTGAAGGGCGAGGCGCCGGCGAACAGCGGAGCACCGGCC GGGGCCGCGGGCCGAGCCAAGGGCGAGTCCCGTATCCGGCGGCCGATGAACGCTTT CATGGTGTGGGCTAAGGACGAGCGCAAGCGGCTGGCGCAGCAGAATCCAGACCTGCA CAACGCCGAGTTGAGCAAGATGCTGGGCAAGTCGTGGAAGGCGCTGACGCTGGCGGA GAAGCGGCCCTTCGTGGAGGAGGCAGAGCGGCTGCGCGTGCAGCACATGCAGGACC ACCCCAACTACAAGTACCGGCCGCGGCGGCGCAAGCAGGTGAAGCGGCTGAAGCGG GTGGAGGGCGGCTTCCTGCACGGCCTGGCTGAGCCGCAGGCGGCCGCGCTGGGCCC CGAGGGCGGCCGCGTGGCCATGGACGGCCTGGGCCTCCAGTTCCCCGAGCAGGGCT TCCCCGCCGGCCCGCCGCTGCTGCCTCCGCACATGGGCGGCCACTACCGCGACTGC CAGAGTCTGGGCGCGCCTCCGCTCGACGGCTACCCGTTGCCCACGCCCGACACGTCC CCGCTGGACGGCGTGGACCCCGACCCGGCTTTCTTCGCCGCCCCGATGCCCGGGGA CTGCCCGGCGGCCGGCACCTACAGCTACGCGCAGGTCTCGGACTACGCTGGCCCCC CGGAGCCTCCCGCCGGTCCCATGCACCCCCGACTCGGCCCAGAGCCCGCGGGTCCC TCGATTCCGGGCCTCCTGGCGCCACCCAGCGCCCTTCACGTGTACTACGGCGCGATG GGCTCGCCCGGGGCGGGCGGCGGGCGCGGCTTCCAGATGCAGCCGCAACACCAGCA CCAGCACCAGCACCAGCACCACCCCCCGGGCCCCGGACAGCCGTCGCCCCCTCCGG AGGCACTGCCCTGCCGGGACGGCACGGACCCCAGTCAGCCCGCCGAGCTCCTCGGG GAGGTGGACCGCACGGAATTTGAACAGTATCTGCACTTCGTGTGCAAGCCTGAGATGG GCCTCCCCTACCAGGGGCATGACTCCGGTGTGAATCTCCCCGACAGCCACGGGGCCA TTTCCTCGGTGGTGTCCGACGCCAGCTCCGCGGTATATTACTGCAACTATCCTGACGT G (SEQ ID NO:154; NM_022454), or a nucleic acid sequence that hybridizes to a nucleic acid sequence consisting of SEQ ID NO:154 under stringent hybridization conditions.

In some embodiments, SRY-box 18 (SOX18) comprises the amino acid sequence: MQRSPPGYGAQDDPPARRDCAWAPGHGAAADTRGLAAGPAALAAPAAPASPPSPQRSP PRSPEPGRYGLSPAGRGERQAADESRIRRPMNAFMVWAKDERKRLAQQNPDLHNAVLSK MLGKAWKELNAAEKRPFVEEAERLRVQHLRDHPNYKYRPRRKKQARKARRLEPGLLLPGL APPQPPPEPFPAASGSARAFRELPPLGAEFDGLGLPTPERSPLDGLEPGEAAFFPPPAAPE DCALRPFRAPYAPTELSRDPGGCYGAPLAEALRTAPPAAPLAGLYYGTLGTPGPYPGPLSP PPEAPPLESAEPLGPAADLWADVDLTEFDQYLNCSRTRPDAPGLPYHVALAKLGPRAMSC PEESSLISALSDASSAVYYSACISGSGP (SEQ ID NO:155; NP_060889), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:155.

In some embodiments, the nucleic acid sequence encoding SOX18 comprises the nucleic acid sequence: ATGCAGAGATCGCCGCCCGGCTACGGCGCACAGGACGACCCGCCCGCCCGCCGCGA CTGTGCATGGGCCCCGGGACACGGGGCCGCCGCTGACACGCGCGGCCTCGCCGCCG GCCCCGCCGCCCTCGCCGCGCCCGCCGCGCCCGCCTCGCCGCCCAGCCCGCAGCG CAGTCCCCCGCGCAGCCCCGAGCCGGGGCGCTATGGCCTCAGCCCGGCCGGCCGCG GGGAACGCCAGGCGGCAGACGAGTCGCGCATCCGGCGGCCCATGAACGCCTTCATG GTGTGGGCAAAGGACGAGCGCAAGCGGCTGGCTCAGCAGAACCCGGACCTGCACAAC GCGGTGCTCAGCAAGATGCTGGGCAAAGCGTGGAAGGAGCTGAACGCGGCGGAGAA GCGGCCCTTCGTGGAGGAAGCCGAACGGCTGCGCGTGCAGCACTTGCGCGACCACC CCAACTACAAGTACCGGCCGCGCCGCAAGAAGCAGGCGCGCAAGGCCCGGCGGCTG GAGCCCGGCCTCCTGCTCCCGGGATTAGCGCCCCCGCAGCCACCGCCCGAGCCTTTC CCCGCGGCGTCTGGCTCGGCTCGCGCCTTCCGCGAGCTGCCCCCGCTGGGCGCCGA GTTCGACGGCCTGGGGCTGCCCACGCCCGAGCGCTCGCCTCTGGACGGCCTGGAGC CCGGCGAGGCTGCCTTCTTCCCACCGCCCGCGGCGCCCGAGGACTGCGCGCTGCGG CCCTTCCGCGCGCCCTACGCGCCCACCGAGTTGTCGCGGGACCCCGGCGGTTGCTAC GGGGCTCCCCTGGCGGAGGCGCTCAGGACCGCGCCCCCCGCGGCGCCGCTCGCTG GCCTGTACTACGGCACCCTGGGCACGCCCGGCCCGTACCCCGGCCCGCTGTCGCCG CCGCCCGAGGCCCCGCCGCTGGAGAGCGCCGAGCCGCTGGGGCCCGCCGCCGATCT GTGGGCCGACGTGGACCTCACCGAGTTCGACCAGTACCTCAACTGCAGCCGGACTCG GCCCGACGCCCCCGGGCTCCCGTACCACGTGGCACTGGCCAAACTGGGCCCGCGCG CCATGTCCTGCCCAGAGGAGAGCAGCCTGATCTCCGCGCTGTCGGACGCCAGCAGCG CGGTCTATTACAGCGCGTGCATCTCCGGCAGCGGACCG (SEQ ID NO:156; NM_018419), or a nucleic acid sequence that hybridizes to a nucleic acid sequence consisting of SEQ ID NO:156 under stringent hybridization conditions.

In some embodiments, SRY-box 15 (SOX15) comprises the amino acid sequence: MALPGSSQDQAWSLEPPAATAAASSSSGPQEREGAGSPAAPGTLPLEKVKRPMNAFMVW SSAQRRQMAQQNPKMHNSEISKRLGAQWKLLDEDEKRPFVEEAKRLRARHLRDYPDYKY RPRRKAKSSGAGPSRCGQGRGNLASGGPLWGPGYATTQPSRGFGYRPPSYSTAYLPGS YGSSHCKLEAPSPCSLPQSDPRLQGELLPTYTHYLPPGSPTPYNPPLAGAPMPLTHL (SEQ ID NO:157; NP_008873), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:157.

In some embodiments, the nucleic acid sequence encoding SOX15 comprises the nucleic acid sequence: ATGGCGCTACCAGGCTCCTCACAGGACCAGGCCTGGAGCCTGGAGCCTCCGGCTGCC ACGGCTGCTGCCTCCTCATCTTCGGGACCCCAGGAGCGGGAGGGCGCTGGGAGCCC CGCGGCCCCCGGGACGCTGCCCCTGGAGAAGGTGAAGCGGCCGATGAACGCGTTCA TGGTGTGGAGCTCCGCTCAGCGCCGCCAGATGGCGCAGCAGAACCCCAAGATGCACA ACTCCGAGATCTCCAAGCGCCTGGGCGCGCAGTGGAAGCTGCTGGACGAGGACGAGA AGCGGCCCTTCGTGGAGGAGGCCAAGCGGCTCCGCGCCCGACACCTGCGCGACTAC CCCGACTACAAGTACCGGCCTCGGCGCAAGGCCAAGAGCTCGGGCGCCGGACCTTCC CGCTGCGGACAGGGAAGAGGCAACCTGGCCAGCGGCGGCCCGCTCTGGGGGCCGG GGTACGCGACCACCCAACCGAGCAGAGGCTTTGGGTACAGACCCCCCAGCTACTCGA CAGCCTACCTGCCTGGCAGCTATGGCTCTTCCCACTGCAAACTGGAAGCCCCCTCACC GTGCTCCCTCCCTCAGAGTGACCCTAGGCTCCAGGGGGAACTGCTGCCCACCTATACC CACTACCTGCCCCCTGGCTCTCCCACTCCATACAACCCTCCCCTTGCTGGTGCCCCCA TGCCCCTAACCCACCTC (SEQ ID NO:158; NM_006942), or a nucleic acid sequence that hybridizes to a nucleic acid sequence consisting of SEQ ID NO:158 under stringent hybridization conditions.

In some embodiments, SRY-box 30 (SOX30) comprises the amino acid sequence: MERARPEPPPQPRPLRPAPPPLPVEGTSFWAAAMEPPPSSPTLSAAASATLASSCGEAVA SGLQPAVRRLLQVKPEQVLLLPQPQAQNEEAAASSAQARLLQFRPDLRLLQPPTASDGAT SRPELHPVQPLALHVKAKKQKLGPSLDQSVGPRGAVETGPRASRVVKLEGPGPALGYFRG DEKGKLEAEEVMRDSMQGGAGKSPAAIREGVIKTEEPERLLEDCRLGAEPASNGLVHGSA EVILAPTSGAFGPHQQDLRIPLTLHTVPPGARIQFQGAPPSELIRLTKVPLTPVPTKMQSLLE PSVKIETKDVPLTVLPSDAGIPDTPFSKDRNGHVKRPMNAFMVWARIHRPALAKANPAANN AEISVQLGLEWNKLSEEQKKPYYDEAQKIKEKHREEFPGWVYQPRPGKRKRFPLSVSNVF SGTTKNIISTNPTTVYPYRSPTYSVVIPSLQNPITHPVGETSPAIQLPTPAVQSPSPVTLFQPS VSSAAQVAVQDPSLPVYPALPPQRFTGPSQTDTHQLHSEAT HTVKQPTPVSLESANRISSS ASTAHARFATSTIQPPREYSSVSPCPRSAPIPQASPIPHPHVYQPPPLGH PATLFGTPPRFS FHHPYFLPGPHYFPSSTCPYSRPPFGYGNFPSSMPECLSYYEDRYPKHEGIFSTLNRDYSF RDYSSECTHSENSRSCENMNGTSYYNSHSHSGEENLNPVPQLDIGTLENVFTAPTSTPSSI QQVNVTDSDEEEEEKVLRDL (SEQ ID NO:159; NP_848511), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:159.

In some embodiments, the nucleic acid sequence encoding SOX30 comprises the nucleic acid sequence: ATGGAGAGAGCCAGACCCGAGCCGCCGCCTCAGCCGCGCCCGTTGCGTCCCGCTCC GCCCCCGCTGCCGGTCGAGGGCACCTCCTTTTGGGCAGCAGCCATGGAGCCCCCTCC GTCGTCTCCCACACTGAGCGCGGCAGCCAGTGCGACCTTGGCCTCGTCGTGCGGGGA GGCAGTGGCGTCCGGCTTACAGCCCGCGGTGCGGCGGCTGCTGCAGGTGAAGCCAG AGCAGGTGTTGCTGCTACCACAGCCTCAGGCCCAGAACGAGGAAGCCGCTGCCTCGT CCGCGCAGGCGCGGCTGTTGCAGTTCAGGCCCGACCTGCGGCTCCTGCAGCCGCCG ACAGCGTCAGACGGCGCCACCTCCAGGCCCGAGTTGCACCCGGTGCAGCCCCTGGC GCTGCATGTCAAGGCCAAGAAGCAGAAGCTGGGGCCCAGCCTGGATCAGTCAGTGGG GCCTCGAGGGGCCGTCGAAACCGGTCCTAGAGCCTCCAGGGTGGTCAAGTTGGAAGG CCCCGGGCCGGCCCTCGGCTACTTCCGAGGGGACGAGAAGGGCAAGCTGGAGGCGG AGGAGGTCATGAGAGACTCGATGCAAGGCGGGGCAGGCAAAAGCCCGGCAGCCATCC GAGAAGGTGTGATCAAAACGGAGGAACCCGAGAGACTCCTCGAGGACTGCAGGCTCG GCGCGGAGCCCGCGTCCAATGGCCTGGTTCATGGCAGCGCGGAGGTCATCTTGGCCC CAACGTCCGGTGCCTTTGGGCCGCACCAGCAAGACCTTAGGATCCCTTTGACGCTCCA CACGGTCCCCCCTGGGGCCCGGATCCAGTTTCAGGGAGCTCCGCCTTCAGAGCTGAT AAGATTGACCAAGGTCCCCCTGACACCAGTGCCTACTAAAATGCAGTCCCTACTGGAG CCTTCTGTAAAAATTGAAACCAAAGATGTCCCGCTCACCGTGTTGCCCTCAGATGCAGG CATACCAGATACTCCCTTCAGTAAGGACAGAAATGGTCATGTGAAGCGACCCATGAAC GCATTTATGGTTTGGGCAAGGATCCACCGACCAGCACTAGCCAAAGCTAACCCAGCAG CCAACAATGCAGAAATCAGTGTCCAGCTTGGGTTAGAGTGGAACAAACTTAGTGAAGAA CAAAAGAAACCCTATTACGATGAAGCACAAAAGATTAAGGAAAAGCACAGAGAGGAATT TCCTGGTTGGGTTTATCAGCCTCGTCCAGGGAAGCGAAAACGATTCCCTCTAAGTGTTT CCAATGTATTTTCTGGTACCACAAAGAATATCATCTCTACAAATCCTACAACAGTTTATC CTTACCGCTCACCTACGTACTCTGTGGTAATTCCCAGCCTACAGAATCCCATCACTCAT CCAGTTGGTGAAACCTCACCTGCTATCCAGCTGCCCACACCTGCAGTCCAGAGCCCAA GCCCTGTCACACTTTTCCAGCCCAGCGTCTCCAGTGCTGCTCAGGTGGCTGTCCAGGA TCCAAGTCTACCTGTCTATCCAGCACTCCCACCCCAACGCTTTACTGGGCCTTCCCAAA CAGACACTCATCAGCTGCATTCTGAAGCCACTCACACTGTGAAGCAACCCACTCCTGTC TCTCTAGAGAGCGCCAACAGGATTTCAAGTAGTGCAAGTACTGCCCATGCCAGATTTG CAACTTCGACCATCCAACCTCCTAGGGAGTATTCCAGCGTTTCCCCTTGTCCCAGAAGT GCTCCAATCCCCCAGGCTTCTCCCATTCCACACCCACATGTCTACCAGCCCCCTCCCC TTGGCCATCCAGCCACACTGTTCGGGACACCACCAAGATTCTCTTTTCATCACCCTTAC TTCCTACCCGGACCTCACTACTTCCCATCAAGTACATGCCCTTACAGTCGGCCTCCCTT TGGCTATGGAAATTTTCCGAGTTCAATGCCAGAATGCCTTAGTTATTATGAAGACAGGT ACCCAAAACATGAGGGTATCTTTTCAACTTTAAATAGAGACTATTCTTTTAGAGACTACT CAAGTGAATGCACACACAGTGAAAATTCTCGGAGTTGTGAGAACATGAATGGAACTTCT TACTATAACAGTCATAGCCACAGTGGGGAAGAAAACTTAAACCCTGTGCCTCAGCTGGA CATTGGAACCTTGGAGAATGTCTTCACAGCCCCGACATCAACTCCTTCTAGCATCCAGC AAGTCAATGTCACCGACAGTGATGAGGAGGAAGAAGAAAAAGTGCTCAGGGATTTA (SEQ ID NO:160; NM_178424), or a nucleic acid sequence that hybridizes to a nucleic acid sequence consisting of SEQ ID NO:160 under stringent hybridization conditions.

In some embodiments, Notochord Homeobox (NOTO) comprises the amino acid sequence: MPSPRPRGSPPPAPSGSRVRPPRSGRSPAPRSPTGPNTPRAPGRFESPFSVEAILARPDP CAPAASQPSGSACVHPAFWTAASLCATGGLPWACPTSWLPAYLSVGFYPVPGPRVAPVC GLLGFGVTGLELAHCSGLWAFPDWAPTEDLQDTERQQKRVRTMFNLEQLEELEKVFAKQ HNLVGKKRAQLAARLKLTENQVRVWFQNRRVKYQKQQKLRAAVTSAEAASLDEPSSSSIA SIQSDDAESGVDG (SEQ ID NO:161; NP_001127934), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:161.

In some embodiments, the nucleic acid sequence encoding NOTO comprises the nucleic acid sequence: ATGCCTAGCCCCAGGCCGCGAGGCAGCCCGCCACCCGCTCCCTCGGGCTCTCGGGT CCGACCTCCGCGCTCTGGCCGCTCTCCGGCGCCCAGGTCCCCTACTGGCCCGAACAC GCCCCGCGCTCCCGGACGCTTCGAGTCCCCTTTCTCGGTCGAGGCCATCCTGGCGAG GCCCGACCCCTGCGCGCCGGCGGCCTCCCAGCCGTCGGGCTCCGCCTGCGTCCACC CGGCCTTCTGGACCGCTGCTTCCCTGTGCGCCACCGGGGGTCTGCCCTGGGCTTGCC CGACATCGTGGCTGCCCGCCTACCTGAGCGTAGGTTTTTACCCTGTGCCAGGGCCGC GCGTGGCTCCCGTCTGCGGCCTGCTGGGCTTCGGCGTCACAGGGTTGGAGCTGGCTC ACTGCTCAGGACTCTGGGCCTTCCCAGACTGGGCCCCAACGGAGGACCTACAGGACA CTGAGAGACAGCAAAAGAGAGTCCGAACTATGTTTAACTTGGAGCAGCTGGAAGAGTT GGAGAAAGTGTTTGCAAAACAGCACAATCTGGTGGGGAAGAAGAGAGCCCAGCTGGC AGCTCGGCTCAAACTTACAGAGAACCAGGTGAGAGTCTGGTTCCAGAACCGCAGGGTC AAGTATCAGAAGCAGCAAAAGCTGAGGGCAGCAGTTACATCTGCCGAGGCTGCCTCCC TGGATGAGCCTTCCAGCAGCTCCATCGCCAGTATCCAGAGTGATGATGCCGAGTCAGG AGTGGACGGC (SEQ ID NO:162; NM_001134462), or a nucleic acid sequence that hybridizes to a nucleic acid sequence consisting of SEQ ID NO:162 under stringent hybridization conditions.

In some embodiments, Tenomodulin (TNMD) comprises the amino acid sequence: MAKNPPENCEDCHILNAEAFKSKKICKSLKICGLVFGILTLTLIVLFWGSKHFWPEVPKKAYD MEHTFYSSGEKKKIYMEIDPVTRTEIFRSGNGTDETLEVHDFKNGYTGIYFVGLQKCFIKTQI KVIPEFSEPEEEIDENEEITTTFFEQSVIWVPAEKPIENRDFLKNSKILEICDNVTMYWINPTLI SVSELQDFEEEGEDLHFPANEKKGIEQNEQWVVPQVKVEKTRHARQASEEELPINDYTEN GIEFDPMLDERGYCCIYCRRGNRYCRRVCEPLLGYYPYPYCYQGGRVICRVIMPCNWWVA RMLGRV (SEQ ID NO:163; NP_071427), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:163.

In some embodiments, the nucleic acid sequence encoding TNMD comprises the nucleic acid sequence: ATGGCAAAGAATCCTCCAGAGAATTGTGAAGACTGTCACATTCTAAATGCAGAAGCTTT TAAATCCAAGAAAATATGTAAATCACTTAAGATTTGTGGACTGGTGTTTGGTATCCTGAC CCTAACTCTAATTGTCCTGTTTTGGGGGAGCAAGCACTTCTGGCCGGAGGTACCCAAA AAAGCCTATGACATGGAGCACACTTTCTACAGCAGTGGAGAGAAGAAGAAGATTTACAT GGAAATTGATCCTGTGACCAGAACTGAAATATTCAGAAGCGGAAATGGCACTGATGAAA CATTGGAAGTACACGACTTTAAAAACGGATACACTGGCATCTACTTCGTGGGTCTTCAA AAATGTTTTATCAAAACTCAGATTAAAGTGATTCCTGAATTTTCTGAACCAGAAGAGGAA ATAGATGAGAATGAAGAAATTACCACAACTTTCTTTGAACAGTCAGTGATTTGGGTCCC AGCAGAAAAGCCTATTGAAAACCGAGATTTTCTTAAAAATTCCAAAATTCTGGAGATTTG TGATAACGTGACCATGTATTGGATCAATCCCACTCTAATATCAGTTTCTGAGTTACAAGA CTTTGAGGAGGAGGGAGAAGATCTTCACTTTCCTGCCAACGAAAAAAAAGGGATTGAA CAAAATGAACAGTGGGTGGTCCCTCAAGTGAAAGTAGAGAAGACCCGTCACGCCAGAC AAGCAAGTGAGGAAGAACTTCCAATAAATGACTATACTGAAAATGGAATAGAATTTGAT CCCATGCTGGATGAGAGAGGTTATTGTTGTATTTACTGCCGTCGAGGCAACCGCTATTG CCGCCGCGTCTGTGAACCTTTACTAGGCTACTACCCATATCCATACTGCTACCAAGGAG GACGAGTCATCTGTCGTGTCATCATGCCTTGTAACTGGTGGGTGGCCCGCATGCTGGG GAGGGTC (SEQ ID NO:164; NM_022144), or a nucleic acid sequence that hybridizes to a nucleic acid sequence consisting of SEQ ID NO:164 under stringent hybridization conditions.

In some embodiments, Human nuclear factor of activated T-cells, cytoplasmic, calcineurin-dependent 1 (NFATc1) comprises the amino acid sequence: MPSTSFPVPSKFPLGPAAAVFGRGETLGPAPRAGGTMKSAEEEHYGYASSNVSPALPLPT AHSTLPAPCHNLQTSTPGIIPPADHPSGYGAALDGGPAGYFLSSGHTRPDGAPALESPRIEI TSCLGLYHNNNQFFHDVEVEDVLPSSKRSPSTATLSLPSLEAYRDPSCLSPASSLSSRSCN SEASSYESNYSYPYASPQTSPWQSPCVSPKTTDPEEGFPRGLGACTLLGSPRHSPSTSPR ASVTEESWLGARSSRPASPCNKRKYSLNGRQPPYSPHHSPTPSPHGSPRVSVTDDSWLG NTTQYTSSAIVAAINALTTDSSLDLGDGVPVKSRKTTLEQPPSVALKVEPVGEDLGSPPPPA DFAPEDYSSFQHIRKGGFCDQYLAVPQHPYQWAKPKPLSPTSYMSPTLPALDWQLPSHSG PYELRIEVQPKSHHRAHYETEGSRGAVKASAGGHPIVQLHGYLENEPLMLQLFIGTADDRL LRPHAFYQVHRITGKTVSTTSHEAILSNTKVLEIPLLPENSMRAVIDCAGILKLRNSDIELRKG ETDIGRKNTRVRLVFRVHVPQPSGRTLSLQVASNPIECSQRSAQELPLVEKQSTDSYPVVG GKKMVLSGHNFLQDSKVIFVEKAPDGHHVWEMEAKTDRDLCKPNSLVVEIPPFRNQRITSP VHVSFYVCNGKRKRSQYQRFTYLPANGNAIFLTVSREHERVGCFF (SEQ ID NO:165; NP_765978), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:165.

In some embodiments, the nucleic acid sequence encoding NFATc1 comprises the nucleic acid sequence: ATGCCAAGCACCAGCTTTCCAGTCCCTTCCAAGTTTCCACTTGGCCCTGCGGCTGCGG TCTTCGGGAGAGGAGAAACTTTGGGGCCCGCGCCGCGCGCCGGCGGCACCATGAAG TCAGCGGAGGAAGAACACTATGGCTATGCATCCTCCAACGTCAGCCCCGCCCTGCCG CTCCCCACGGCGCACTCCACCCTGCCGGCCCCGTGCCACAACCTTCAGACCTCCACA CCGGGCATCATCCCGCCGGCGGATCACCCCTCGGGGTACGGGGCAGCTTTGGACGG TGGGCCCGCGGGCTACTTCCTCTCCTCCGGCCACACCAGGCCTGATGGGGCCCCTGC CCTGGAGAGTCCTCGCATCGAGATAACCTCGTGCTTGGGCCTGTACCACAACAATAAC CAGTTTTTCCACGATGTGGAGGTGGAAGACGTCCTCCCTAGCTCCAAACGGTCCCCCT CCACGGCCACGCTGAGTCTGCCCAGCCTGGAGGCCTACAGAGACCCCTCGTGCCTGA GCCCGGCCAGCAGCCTGTCCTCCCGGAGCTGCAACTCAGAGGCCTCCTCCTACGAGT CCAACTACTCGTACCCGTACGCGTCCCCCCAGACGTCGCCATGGCAGTCTCCCTGCGT GTCTCCCAAGACCACGGACCCCGAGGAGGGCTTTCCCCGCGGGCTGGGGGCCTGCA CACTGCTGGGTTCCCCGCGGCACTCCCCCTCCACCTCGCCCCGCGCCAGCGTCACTG AGGAGAGCTGGCTGGGTGCCCGCTCCTCCAGACCCGCGTCCCCGTGCAACAAGAGGA AGTACAGCCTCAACGGCCGGCAGCCGCCCTACTCACCCCACCACTCGCCCACGCCGT CCCCGCACGGCTCCCCGCGGGTCAGCGTGACCGACGACTCGTGGTTGGGCAACACCA CCCAGTACACCAGCTCGGCCATCGTGGCCGCCATCAACGCGCTGACCACCGACAGCA GCCTGGACCTGGGAGATGGCGTCCCTGTCAAGTCCCGCAAGACCACCCTGGAGCAGC CGCCCTCAGTGGCGCTCAAGGTGGAGCCCGTCGGGGAGGACCTGGGCAGCCCCCCG CCCCCGGCCGACTTCGCGCCCGAAGACTACTCCTCTTTCCAGCACATCAGGAAGGGC GGCTTCTGCGACCAGTACCTGGCGGTGCCGCAGCACCCCTACCAGTGGGCGAAGCCC AAGCCCCTGTCCCCTACGTCCTACATGAGCCCGACCCTGCCCGCCCTGGACTGGCAG CTGCCGTCCCACTCAGGCCCGTATGAGCTTCGGATTGAGGTGCAGCCCAAGTCCCAC CACCGAGCCCACTACGAGACGGAGGGCAGCCGGGGGGCCGTGAAGGCGTCGGCCG GAGGACACCCCATCGTGCAGCTGCATGGCTACTTGGAGAATGAGCCGCTGATGCTGC AGCTTTTCATTGGGACGGCGGACGACCGCCTGCTGCGCCCGCACGCCTTCTACCAGG TGCACCGCATCACAGGGAAGACCGTGTCCACCACCAGCCACGAGGCCATCCTCTCCA ACACCAAAGTCCTGGAGATCCCACTCCTGCCGGAGAACAGCATGCGAGCCGTCATTGA CTGTGCCGGAATCCTGAAACTCAGAAACTCCGACATTGAACTTCGGAAAGGAGAGACG GACATCGGGAGGAAGAACACACGGGTACGGCTGGTGTTCCGCGTTCACGTCCCGCAA CCCAGCGGCCGCACGCTGTCCCTGCAGGTGGCCTCCAACCCCATCGAATGCTCCCAG CGCTCAGCTCAGGAGCTGCCTCTGGTGGAGAAGCAGAGCACGGACAGCTATCCGGTC GTGGGCGGGAAGAAGATGGTCCTGTCTGGCCACAACTTCCTGCAGGACTCCAAGGTC ATTTTCGTGGAGAAAGCCCCAGATGGCCACCATGTCTGGGAGATGGAAGCGAAAACTG ACCGGGACCTGTGCAAGCCGAATTCTCTGGTGGTTGAGATCCCGCCATTTCGGAATCA GAGGATAACCAGCCCCGTTCACGTCAGTTTCTACGTCTGCAACGGGAAGAGAAAGCGA AGCCAGTACCAGCGTTTCACCTACCTTCCCGCCAACGGTAACGCCATCTTTCTAACCGT AAGCCGTGAACATGAGCGCGTGGGGTGCTTTTTC (SEQ ID NO:166; NM_172390), or a nucleic acid sequence that hybridizes to a nucleic acid sequence consisting of SEQ ID NO:166 under stringent hybridization conditions.

In some embodiments, Human nuclear factor of activated T-cells, cytoplasmic, calcineurin-dependent 2 (NFATc2) comprises the amino acid sequence: MNAPERQPQPDGGDAPGHEPGGSPQDELDFSILFDYEYLNPNEEEPNAHKVASPPSGPA YPDDVLDYGLKPYSPLASLSGEPPGRFGEPDRVGPQKFLSAAKPAGASGLSPRIEITPSHE LIQAVGPLRMRDAGLLVEQPPLAGVAASPRFTLPVPGFEGYREPLCLSPASSGSSASFISDT FSPYTSPCVSPNNGGPDDLCPQFQNIPAHYSPRTSPIMSPRTSLAEDSCLGRHSPVPRPAS RSSSPGAKRRHSCAEALVALPPGASPQRSRSPSPQPSSHVAPQDHGSPAGYPPVAGSAVI MDALNSLATDSPCGIPPKMWKTSPDPSPVSAAPSKAGLPRHIYPAVEFLGPCEQGERRNS APESILLVPPTWPKPLVPAIPICSIPVTASLPPLEWPLSSQSGSYELRIEVQPKPHHRAHYET EGSRGAVKAPTGGH PVVQLHGYMENKPLGLQIFIGTADERILKPHAFYQVHRITGKTVTTTS YEKIVGNTKVLEIPLEPKNNMRATIDCAGILKLRNADIELRKGETDIGRKNTRVRLVFRVHIPE SSGRIVSLQTASNPIECSQRSAH ELPMVERQDTDSCLVYGGQQMILTGQNFTSESKVVFTE KTTDGQQIWEMEATVDKDKSQPNMLFVEIPEYRNKHIRTPVKVNFYVINGKRKRSQPQHFT YH PVPAIKTEPTDEYDPTLICSPTHGGLGSQPYYPQH PMVAESPSCLVATMAPCQQFRTGL SSPDARYQQQNPAAVLYQRSKSLSPSLLGYQQPALMAAPLSLADAHRSVLVHAGSQGQS SALLHPSPTNQQASPVIHYSPTNQQLRCGSHQEFQHIMYCENFAPGTTRPGPPPVSQGQR LSPGSYPTVIQQQNATSQRAAKNGPPVSDQKEVLPAGVTIKQEQNLDQTYLDDELIDTHLS WIQNIL (SEQ ID NO:167; NP_036472), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:167.

In some embodiments, the nucleic acid sequence encoding NFATc2 comprises the nucleic acid sequence: ATGAACGCCCCCGAGCGGCAGCCCCAACCCGACGGCGGGGACGCCCCAGGCCACGA GCCTGGGGGCAGCCCCCAAGACGAGCTTGACTTCTCCATCCTCTTCGACTATGAGTAT TTGAATCCGAACGAAGAAGAGCCGAATGCACATAAGGTCGCCAGCCCACCCTCCGGAC CCGCATACCCCGATGATGTCCTGGACTATGGCCTCAAGCCATACAGCCCCCTTGCTAG TCTCTCTGGCGAGCCCCCCGGCCGATTCGGAGAGCCGGATAGGGTAGGGCCGCAGAA GTTTCTGAGCGCGGCCAAGCCAGCAGGGGCCTCGGGCCTGAGCCCTCGGATCGAGAT CACTCCGTCCCACGAACTGATCCAGGCAGTGGGGCCCCTCCGCATGAGAGACGCGGG CCTCCTGGTGGAGCAGCCGCCCCTGGCCGGGGTGGCCGCCAGCCCGAGGTTCACCC TGCCCGTGCCCGGCTTCGAGGGCTACCGCGAGCCGCTTTGCTTGAGCCCCGCTAGCA GCGGCTCCTCTGCCAGCTTCATTTCTGACACCTTCTCCCCCTACACCTCGCCCTGCGT CTCGCCCAATAACGGCGGGCCCGACGACCTGTGTCCGCAGTTTCAAAACATCCCTGCT CATTATTCCCCCAGAACCTCGCCAATAATGTCACCTCGAACCAGCCTCGCCGAGGACA GCTGCCTGGGCCGCCACTCGCCCGTGCCCCGTCCGGCCTCCCGCTCCTCATCGCCTG GTGCCAAGCGGAGGCATTCGTGCGCCGAGGCCTTGGTTGCCCTGCCGCCCGGAGCCT CACCCCAGCGCTCCCGGAGCCCCTCGCCGCAGCCCTCATCTCACGTGGCACCCCAGG ACCACGGCTCCCCGGCTGGGTACCCCCCTGTGGCTGGCTCTGCCGTGATCATGGATG CCCTGAACAGCCTCGCCACGGACTCGCCTTGTGGGATCCCCCCCAAGATGTGGAAGA CCAGCCCTGACCCCTCGCCGGTGTCTGCCGCCCCATCCAAGGCCGGCCTGCCTCGCC ACATCTACCCGGCCGTGGAGTTCCTGGGGCCCTGCGAGCAGGGCGAGAGGAGAAACT CGGCTCCAGAATCCATCCTGCTGGTTCCGCCCACTTGGCCCAAGCCGCTGGTGCCTG CCATTCCCATCTGCAGCATCCCAGTGACTGCATCCCTCCCTCCACTTGAGTGGCCGCT GTCCAGTCAGTCAGGCTCTTACGAGCTGCGGATCGAGGTGCAGCCCAAGCCACATCA CCGGGCCCACTATGAGACAGAAGGCAGCCGAGGGGCTGTCAAAGCTCCAACTGGAGG CCACCCTGTGGTTCAGCTCCATGGCTACATGGAAAACAAGCCTCTGGGACTTCAGATC TTCATTGGGACAGCTGATGAGCGGATCCTTAAGCCGCACGCCTTCTACCAGGTGCACC GAATCACGGGGAAAACTGTCACCACCACCAGCTATGAGAAGATAGTGGGCAACACCAA AGTCCTGGAGATACCCTTGGAGCCCAAAAACAACATGAGGGCAACCATCGACTGTGCG GGGATCTTGAAGCTTAGAAACGCCGACATTGAGCTGCGGAAAGGCGAGACGGACATT GGAAGAAAGAACACGCGGGTGAGACTGGTTTTCCGAGTTCACATCCCAGAGTCCAGTG GCAGAATCGTCTCTTTACAGACTGCATCTAACCCCATCGAGTGCTCCCAGCGATCTGCT CACGAGCTGCCCATGGTTGAAAGACAAGACACAGACAGCTGCCTGGTCTATGGCGGC CAGCAAATGATCCTCACGGGGCAGAACTTTACATCCGAGTCCAAAGTTGTGTTTACTGA GAAGACCACAGATGGACAGCAAATTTGGGAGATGGAAGCCACGGTGGATAAGGACAA GAGCCAGCCCAACATGCTTTTTGTTGAGATCCCTGAATATCGGAACAAGCATATCCGCA CACCTGTAAAAGTGAACTTCTACGTCATCAATGGGAAGAGAAAACGAAGTCAGCCTCAG CACTTTACCTACCACCCAGTCCCAGCCATCAAGACGGAGCCCACGGATGAATATGACC CCACTCTGATCTGCAGCCCCACCCATGGAGGCCTGGGGAGCCAGCCTTACTACCCCC AGCACCCGATGGTGGCCGAGTCCCCCTCCTGCCTCGTGGCCACCATGGCTCCCTGCC AGCAGTTCCGCACGGGGCTCTCATCCCCTGACGCCCGCTACCAGCAACAGAACCCAG CGGCCGTACTCTACCAGCGGAGCAAGAGCCTGAGCCCCAGCCTGCTGGGCTATCAGC AGCCGGCCCTCATGGCCGCCCCGCTGTCCCTTGCGGACGCTCACCGCTCTGTGCTGG TGCACGCCGGCTCCCAGGGCCAGAGCTCAGCCCTGCTCCACCCCTCTCCGACCAACC AGCAGGCCTCGCCTGTGATCCACTACTCACCCACCAACCAGCAGCTGCGCTGCGGAA GCCACCAGGAGTTCCAGCACATCATGTACTGCGAGAATTTCGCACCAGGCACCACCAG ACCTGGCCCGCCCCCGGTCAGTCAAGGTCAGAGGCTGAGCCCGGGTTCCTACCCCAC AGTCATTCAGCAGCAGAATGCCACGAGCCAAAGAGCCGCCAAAAACGGACCCCCGGT CAGTGACCAAAAGGAAGTATTACCTGCGGGGGTGACCATTAAACAGGAGCAGAACTTG GACCAGACCTACTTGGATGATGAGCTGATAGACACACACCTTAGCTGGATACAAAACAT ATTA (SEQ ID NO:168; NM_012340), or a nucleic acid sequence that hybridizes to a nucleic acid sequence consisting of SEQ ID NO:168 under stringent hybridization conditions.

In some embodiments, Human nuclear factor of activated T-cells, cytoplasmic, calcineurin-dependent 3 (NFATc3) comprises the amino acid sequence: MTTANCGAHDELDFKLVFGEDGAPAPPPPGSRPADLEPDDCASIYIFNVDPPPSTLTTPLCL PHHGLPSHSSVLSPSFQLQSHKNYEGTCEIPESKYSPLGGPKPFECPSIQITSISPNCHQEL DAHEDDLQINDPEREFLERPSRDHLYLPLEPSYRESSLSPSPASSISSRSWFSDASSCESLS HIYDDVDSELNEAAARFTLGSPLTSPGGSPGGCPGEETWHQQYGLGHSLSPRQSPCHSP RSSVTDENWLSPRPASGPSSRPTSPCGKRRHSSAEVCYAGSLSPHHSPVPSPGHSPRGS VTEDTWLNASVHGGSGLGPAVFPFQYCVETDIPLKTRKTSEDQAAILPGKLELCSDDQGSL SPARETSIDDGLGSQYPLKKDSCGDQFLSVPSPFTWSKPKPGHTPIFRTSSLPPLDWPLPA HFGQCELKIEVQPKTHHRAHYETEGSRGAVKASTGGHPVVKLLGYNEKPINLQMFIGTADD RYLRPHAFYQVHRITGKTVATASQEIIIASTKVLEIPLLPENNMSASIDCAGILKLRNSDIELRK GETDIGRKNTRVRLVFRVHIPQPSGKVLSLQIASIPVECSQRSAQELPHIEKYSINSCSVNGG HEMVVTGSNFLPESKIIFLEKGQDGRPQWEVEGKIIREKCQGAHIVLEVPPYHNPAVTAAVQ VHFYLCNGKRKKSQSQRFTYTPVLMKQEHREEIDLSSVPSLPVPHPAQTQRPSSDSGCSH DSVLSGQRSLICSIPQTYASMVTSSHLPQLQCRDESVSKEQHMIPSPIVHQPFQVTPTPPVG SSYQPMQTNVVYNGPTCLPINAASSQEFDSVLFQQDATLSGLVNLGCQPLSSIPFHSSNSG STGHLLAHTPHSVHTLPHLQSMGYHCSNTGQRSLSSPVADQITGQPSSQLQPITYGPSHS GSATTASPAASHPLASSPLSGPPSPQLQPMPYQSPSSGTASSPSPATRMHSGQHSTQAQ STGQGGLSAPSSLICHSLCDPASFPPDGATVSIKPEPEDREPNFATIGLQDITLDDDQFISDL EHQPSGSAEKWPNHSVLSCPAPFWRI (SEQ ID NO:169; NP_004546), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:169.

In some embodiments, the nucleic acid sequence encoding NFATc3 comprises the nucleic acid sequence: ATGACTACTGCAAACTGTGGCGCCCACGACGAGCTCGACTTCAAACTCGTCTTTGGCG AGGACGGGGCGCCGGCGCCGCCGCCCCCGGGCTCGCGGCCTGCAGATCTTGAGCCA GATGATTGTGCATCCATTTACATCTTTAATGTAGATCCACCTCCATCTACTTTAACCACA CCACTTTGCTTACCACATCATGGATTACCGTCTCACTCTTCTGTTTTGTCACCATCGTTT CAGCTCCAAAGTCACAAAAACTATGAAGGAACTTGTGAGATTCCTGAATCTAAATATAG CCCATTAGGTGGTCCCAAACCCTTTGAGTGCCCAAGTATTCAAATTACATCTATCTCTC CTAACTGTCATCAAGAATTAGATGCACATGAAGATGACCTACAGATAAATGACCCAGAA CGGGAATTTTTGGAAAGGCCTTCTAGAGATCATCTCTATCTTCCTCTTGAGCCATCCTA CCGGGAGTCTTCTCTTAGTCCTAGTCCTGCCAGCAGCATCTCTTCTAGGAGTTGGTTCT CTGATGCATCTTCTTGTGAATCGCTTTCACATATTTATGATGATGTGGACTCAGAGTTGA ATGAAGCTGCAGCCCGATTTACCCTTGGATCCCCTCTGACTTCTCCTGGTGGCTCTCCA GGGGGCTGCCCTGGAGAAGAAACTTGGCATCAACAGTATGGACTTGGACACTCATTAT CACCCAGGCAATCTCCTTGCCACTCTCCTAGATCCAGTGTCACTGATGAGAATTGGCTG AGCCCCAGGCCAGCCTCAGGACCCTCATCAAGGCCCACATCCCCCTGTGGGAAACGG AGGCACTCCAGTGCTGAAGTTTGTTATGCTGGGTCCCTTTCACCCCATCACTCACCTGT TCCTTCACCTGGTCACTCCCCCAGGGGAAGTGTGACAGAAGATACGTGGCTCAATGCT TCTGTCCATGGTGGGTCAGGCCTTGGCCCTGCAGTTTTTCCATTTCAGTACTGTGTAGA GACTGACATCCCTCTCAAAACAAGGAAAACTTCTGAAGATCAAGCTGCCATACTACCAG GAAAATTAGAGCTGTGTTCAGATGACCAAGGGAGTTTATCACCAGCCCGGGAGACTTC AATAGATGATGGCCTTGGATCTCAGTATCCTTTAAAGAAAGATTCATGTGGTGATCAGTT TCTTTCAGTTCCTTCACCCTTTACCTGGAGCAAACCAAAGCCTGGCCACACCCCTATAT TTCGCACATCTTCATTACCTCCACTAGACTGGCCTTTACCAGCTCATTTTGGACAATGTG AACTGAAAATAGAAGTGCAACCTAAAACTCATCATCGAGCCCATTATGAAACTGAAGGT AGCCGAGGGGCAGTAAAAGCATCTACTGGGGGACATCCTGTTGTGAAGCTCCTGGGC TATAACGAAAAGCCAATAAATCTACAAATGTTTATTGGGACAGCAGATGATCGATATTTA CGACCTCATGCATTTTACCAGGTGCATCGAATCACTGGGAAGACAGTCGCTACTGCAA GCCAAGAGATAATAATTGCCAGTACAAAAGTTCTGGAAATTCCACTTCTTCCTGAAAATA ATATGTCAGCCAGTATTGATTGTGCAGGTATTTTGAAACTCCGCAATTCAGATATAGAAC TTCGAAAAGGAGAAACTGATATTGGCAGAAAGAATACTAGAGTACGACTTGTGTTTCGT GTACACATCCCACAGCCCAGTGGAAAAGTCCTTTCTCTGCAGATAGCCTCTATACCCGT TGAGTGCTCCCAGCGGTCTGCTCAAGAACTTCCTCATATTGAGAAGTACAGTATCAACA GTTGTTCTGTAAATGGAGGTCATGAAATGGTTGTGACTGGATCTAATTTTCTTCCAGAAT CCAAAATCATTTTTCTTGAAAAAGGACAAGATGGACGACCTCAGTGGGAGGTAGAAGG GAAGATAATCAGGGAAAAATGTCAAGGGGCTCACATTGTCCTTGAAGTTCCTCCATATC ATAACCCAGCAGTTACAGCTGCAGTGCAGGTGCACTTTTATCTTTGCAATGGCAAGAGG AAAAAAAGCCAGTCTCAACGTTTTACTTATACACCAGTTTTGATGAAGCAAGAACACAGA GAAGAGATTGATTTGTCTTCAGTTCCATCTTTGCCTGTGCCTCATCCTGCTCAGACCCA GAGGCCTTCCTCTGATTCAGGGTGTTCACATGACAGTGTACTGTCAGGACAGAGAAGT TTGATTTGCTCCATCCCACAAACATATGCATCCATGGTGACCTCATCCCATCTGCCACA GTTGCAGTGTAGAGATGAGAGTGTTAGTAAAGAACAGCATATGATTCCTTCTCCAATTG TACACCAGCCTTTTCAAGTCACACCAACACCTCCTGTGGGGTCTTCCTATCAGCCTATG CAAACTAATGTTGTGTACAATGGACCAACTTGTCTTCCTATTAATGCTGCCTCTAGTCAA GAATTTGATTCAGTTTTGTTTCAGCAGGATGCAACTCTTTCTGGTTTAGTGAATCTTGGC TGTCAACCACTGTCATCCATACCATTTCATTCTTCAAATTCAGGCTCAACAGGACATCTC TTAGCCCATACACCTCATTCTGTGCATACCCTGCCTCATCTGCAATCAATGGGATATCA TTGTTCAAATACAGGACAAAGATCTCTTTCTTCTCCAGTGGCTGACCAGATTACAGGTC AGCCTTCGTCTCAGTTACAACCTATTACATATGGTCCTTCACATTCAGGGTCTGCTACAA CAGCTTCCCCAGCAGCTTCTCATCCCTTGGCTAGTTCACCGCTTTCTGGGCCACCATCT CCTCAGCTTCAGCCTATGCCTTACCAATCTCCTAGCTCAGGAACTGCCTCATCACCGTC TCCAGCCACCAGAATGCATTCTGGACAGCACTCAACTCAAGCACAAAGTACGGGCCAG GGGGGTCTTTCTGCACCTTCATCCTTAATATGTCACAGTTTGTGTGATCCAGCGTCATT TCCACCTGATGGGGCAACTGTGAGCATTAAACCTGAACCAGAAGATCGAGAGCCTAAC TTTGCAACCATTGGTCTGCAGGACATCACTTTAGATGATGACCAATTTATATCTGACTTG GAACACCAGCCATCAGGTTCAGCAGAGAAATGGCCTAACCACAGTGTGCTCTCATGTC CAGCTCCTTTCTGGAGAATC (SEQ ID NO:170; NM_004555), or a nucleic acid sequence that hybridizes to a nucleic acid sequence consisting of SEQ ID NO:170 under stringent hybridization conditions.

In some embodiments, Human nuclear factor of activated T-cells, cytoplasmic, calcineurin-dependent 4 (NFATc4) comprises the amino acid sequence: MITTLPSLLPASLASISHRVTNLPSNSLSHNPGLSKPDFPGNSSPGLPSSSSPGRDLGAPAG SMGAASCEDEELEFKLVFGEEKEAPPLGAGGLGEELDSEDAPPCCRLALGEPPPYGAAPI GIPRPPPPRPGMHSPPPRPAPSPGTWESQPARSVRLGGPGGGAGGAGGGRVLECPSIRI TSISPTPEPPAALEDNPDAWGDGSPRDYPPPEGFGGYREAGGQGGGAFFSPSPGSSSLS SWSFFSDASDEAALYAACDEVESELNEAASRFGLGSPLPSPRASPRPWTPEDPWSLYGPS PGGRGPEDSWLLLSAPGPTPASPRPASPCGKRRYSSSGTPSSASPALSRRGSLGEEGSE PPPPPPLPLARDPGSPGPFDYVGAPPAESIPQKTRRTSSEQAVALPRSEEPASCNGKLPLG AEESVAPPGGSRKEVAGMDYLAVPSPLAWSKARIGGHSPIFRTSALPPLDWPLPSQYEQL ELRIEVQPRAHHRAHYETEGSRGAVKAAPGGHPVVKLLGYSEKPLTLQMFIGTADERNLRP HAFYQVH RITGKMVATASYEAVVSGTKVLEMTLLPENNMAANIDCAGILKLRNSDIELRKGE TDIGRKNTRVRLVFRVHVPQGGGKVVSVQAASVPIECSQRSAQELPQVEAYSPSACSVRG GEELVLTGSNFLPDSKVVFIERGPDGKLQWEEEATVNRLQSNEVTLTLTVPEYSNKRVSRP VQVYFYVSNGRRKRSPTQSFRFLPVICKEEPLPDSSLRGFPSASATPFGTDMDFSPPRPPY PSYPHEDPACETPYLSEGFGYGMPPLYPQTGPPPSYRPGLRMFPETRGTTGCAQPPAVS FLPRPFPSDPYGGRGSSFSLGLPFSPPAPFRPPPLPASPPLEGPFPSQSDVHPLPAEGYNK VGPGYGPGEGAPEQEKSRGGYSSGFRDSVPIQGITLEEGGCGTGGCECECVQEIALHVC (SEQ ID NO:171; NP_001129494), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:171.

In some embodiments, the nucleic acid sequence encoding NFATc4 comprises the nucleic acid sequence: ATGATAACCACCCTCCCATCTCTCCTACCCGCCAGCCTCGCCAGTATCTCCC ACCGAGTCACGAATCTCCCATCTAACTCCCTCTCACACAACCCAGGCCTCTCCAAGCCT GACTTTCCCGGAAACTCCAGTCCAGGTCTTCCTTCCTCCTCCAGCCCAGGCCGGGACC TGGGGGCTCCTGCCGGATCCATGGGGGCGGCCAGCTGCGAGGATGAGGAGCTGGAA TTTAAGCTGGTGTTCGGGGAGGAAAAGGAGGCCCCCCCGCTGGGCGCGGGGGGATT GGGGGAAGAACTGGACTCAGAGGATGCCCCGCCATGCTGCCGTCTGGCCTTGGGAGA GCCCCCTCCCTATGGCGCTGCACCTATCGGTATTCCCCGACCTCCACCCCCTCGGCCT GGCATGCATTCGCCACCGCCGCGACCAGCCCCCTCACCTGGCACCTGGGAGAGCCAG CCCGCCAGGTCGGTGAGGCTGGGAGGACCAGGAGGGGGTGCTGGGGGTGCTGGGG GTGGCCGTGTTCTCGAGTGTCCCAGCATCCGCATCACCTCCATCTCTCCCACGCCGGA GCCGCCAGCAGCGCTGGAGGACAACCCTGATGCCTGGGGGGACGGCTCTCCTAGAG ATTACCCCCCACCAGAAGGCTTTGGGGGCTACAGAGAAGCAGGGGGCCAGGGTGGG GGGGCCTTCTTCAGCCCAAGCCCTGGCAGCAGCAGCCTGTCCTCGTGGAGCTTCTTCT CCGATGCCTCTGACGAGGCAGCCCTGTATGCAGCCTGCGACGAGGTGGAGTCTGAGC TAAATGAGGCGGCCTCCCGCTTTGGCCTGGGCTCCCCGCTGCCCTCGCCCCGGGCCT CCCCTCGGCCATGGACCCCCGAAGATCCCTGGAGCCTGTATGGTCCAAGCCCCGGAG GCCGAGGGCCAGAGGATAGCTGGCTACTCCTCAGTGCTCCTGGGCCCACCCCAGCCT CCCCGCGGCCTGCCTCTCCATGTGGCAAGCGGCGCTATTCCAGCTCGGGAACCCCAT CTTCAGCCTCCCCAGCTCTGTCCCGCCGTGGCAGCCTGGGGGAAGAGGGGTCTGAGC CACCTCCACCACCCCCATTGCCTCTGGCCCGGGACCCGGGCTCCCCTGGTCCCTTTG ACTATGTGGGGGCCCCACCAGCTGAGAGCATCCCTCAGAAGACACGGCGGACTTCCA GCGAGCAGGCAGTGGCTCTGCCTCGGTCTGAGGAGCCTGCCTCATGCAATGGGAAGC TGCCCTTGGGAGCAGAGGAGTCTGTGGCTCCTCCAGGAGGTTCCCGGAAGGAGGTGG CTGGCATGGACTACCTGGCAGTGCCCTCCCCACTCGCTTGGTCCAAGGCCCGGATTG GGGGACACAGCCCTATCTTCAGGACCTCTGCCCTACCCCCACTGGACTGGCCTCTGCC CAGCCAATATGAGCAGCTGGAGCTGAGGATCGAGGTACAGCCTAGAGCCCACCACCG GGCCCACTATGAGACAGAAGGCAGCCGTGGAGCTGTCAAAGCTGCCCCTGGCGGTCA CCCCGTAGTCAAGCTCCTAGGCTACAGTGAGAAGCCACTGACCCTACAGATGTTCATC GGCACTGCAGATGAAAGGAACCTGCGGCCTCATGCCTTCTATCAGGTGCACCGTATCA CAGGCAAGATGGTGGCCACGGCCAGCTATGAAGCCGTAGTCAGTGGCACCAAGGTGT TGGAGATGACTCTGCTGCCTGAGAACAACATGGCGGCCAACATTGACTGCGCGGGAAT CCTGAAGCTTCGGAATTCAGACATTGAGCTTCGGAAGGGTGAGACGGACATCGGGCG CAAAAACACACGTGTACGGCTGGTGTTCCGGGTACACGTGCCCCAGGGCGGCGGGAA GGTCGTCTCAGTACAGGCAGCATCGGTGCCCATCGAGTGCTCCCAGCGCTCAGCCCA GGAGCTGCCCCAGGTGGAGGCCTACAGCCCCAGTGCCTGCTCTGTGAGAGGAGGCG AGGAACTGGTACTGACTGGCTCCAACTTCCTGCCAGACTCCAAGGTGGTGTTCATTGA GAGGGGTCCTGATGGGAAGCTGCAATGGGAGGAGGAGGCCACAGTGAACCGACTGCA GAGCAACGAGGTGACGCTGACCCTGACTGTCCCCGAGTACAGCAACAAGAGGGTTTC CCGGCCAGTCCAGGTCTACTTTTATGTCTCCAATGGGCGGAGGAAACGCAGTCCTACC CAGAGTTTCAGGTTTCTGCCTGTGATCTGCAAAGAGGAGCCCCTACCGGACTCATCTC TGCGGGGTTTCCCTTCAGCATCGGCAACCCCCTTTGGCACTGACATGGACTTCTCACC ACCCAGGCCCCCCTACCCCTCCTATCCCCATGAAGACCCTGCTTGCGAAACTCCTTAC CTATCAGAAGGCTTCGGCTATGGCATGCCCCCTCTGTACCCCCAGACGGGGCCCCCA CCATCCTACAGACCGGGCCTGCGGATGTTCCCTGAGACTAGGGGTACCACAGGTTGT GCCCAACCACCTGCAGTTTCCTTCCTTCCCCGCCCCTTCCCTAGTGACCCGTATGGAG GGCGGGGCTCCTCTTTCTCCCTGGGGCTGCCATTCTCTCCGCCAGCCCCCTTTCGGC CGCCTCCTCTTCCTGCATCCCCACCGCTTGAAGGCCCCTTCCCTTCCCAGAGTGATGT GCATCCCCTACCTGCTGAGGGATACAATAAGGTAGGGCCAGGCTATGGCCCTGGGGA GGGGGCTCCGGAGCAGGAGAAATCCAGGGGTGGCTACAGCAGCGGCTTCCGAGACA GTGTCCCTATCCAGGGTATCACGCTGGAGGAAGGTGGGTGTGGGACTGGGGGCTGTG AGTGTGAGTGTGTGCAAGAGATTGCTCTGCATGTTTGC (SEQ ID NO:172; NM_001136022), or a nucleic acid sequence that hybridizes to a nucleic acid sequence consisting of SEQ ID NO:172 under stringent hybridization conditions.

In some embodiments, Human v-ets erythroblastosis virus E26 oncogene homolog (ERG, C-1-1 is a variant) comprises the amino acid sequence: IQTVPDPAAHIKEALSVVSEDQSLFECAYGTPH LAKTEMTASSSSDYGQTSKMSPRVPQQD WLSQPPARVTIKMECNPSQVNGSRNSPDECSVAKGGKMVGSPDTVGMNYGSYMEEKHM PPPNMTTNERRVIVPADPTLWSTDHVRQWLEWAVKEYGLPDVNILLFQNIDGKELCKMTKD DFQRLTPSYNADILLSHLHYLRETPLPHLTSDDVDKALQNSPRLMHARNTGGAAFIFPNTSV YPEATQRITTRPDLPYEPPRRSAWTGHGHPTPQSKAAQPSPSTVPKTEDQRPQLDPYQIL GPTSSRLANPGSGQIQLWQFLLELLSDSSNSSCITWEGTNGEFKMTDPDEVARRWGERKS KPNMNYDKLSRALRYYYDKNIMTKVHGKRYAYKFDFHGIAQALQPHPPESSLYKYPSDLPY MGSYHAHPQKMNFVAPHPPALPVTSSSFFAAPNPYWNSPTGGIYPNTRLPTSHMPSHLGT YY (SEQ ID NO:173; NP_001129626), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:173.

In some embodiments, the nucleic acid sequence encoding ERG comprises the nucleic acid sequence ATGATTCAGACTGTCCCGGACCCAGCAGCTCATATCAAGGAAGCCTTATCAGTTGTGA GTGAGGACCAGTCGTTGTTTGAGTGTGCCTACGGAACGCCACACCTGGCTAAGACAGA GATGACCGCGTCCTCCTCCAGCGACTATGGACAGACTTCCAAGATGAGCCCACGCGTC CCTCAGCAGGATTGGCTGTCTCAACCCCCAGCCAGGGTCACCATCAAAATGGAATGTA ACCCTAGCCAGGTGAATGGCTCAAGGAACTCTCCTGATGAATGCAGTGTGGCCAAAGG CGGGAAGATGGTGGGCAGCCCAGACACCGTTGGGATGAACTACGGCAGCTACATGGA GGAGAAGCACATGCCACCCCCAAACATGACCACGAACGAGCGCAGAGTTATCGTGCC AGCAGATCCTACGCTATGGAGTACAGACCATGTGCGGCAGTGGCTGGAGTGGGCGGT GAAAGAATATGGCCTTCCAGACGTCAACATCTTGTTATTCCAGAACATCGATGGGAAGG AACTGTGCAAGATGACCAAGGACGACTTCCAGAGGCTCACCCCCAGCTACAACGCCGA CATCCTTCTCTCACATCTCCACTACCTCAGAGAGACTCCTCTTCCACATTTGACTTCAGA TGATGTTGATAAAGCCTTACAAAACTCTCCACGGTTAATGCATGCTAGAAACACAGGGG GTGCAGCTTTTATTTTCCCAAATACTTCAGTATATCCTGAAGCTACGCAAAGAATTACAA CTAGGCCAGATTTACCATATGAGCCCCCCAGGAGATCAGCCTGGACCGGTCACGGCC ACCCCACGCCCCAGTCGAAAGCTGCTCAACCATCTCCTTCCACAGTGCCCAAAACTGA AGACCAGCGTCCTCAGTTAGATCCTTATCAGATTCTTGGACCAACAAGTAGCCGCCTTG CAAATCCAGGCAGTGGCCAGATCCAGCTTTGGCAGTTCCTCCTGGAGCTCCTGTCGGA CAGCTCCAACTCCAGCTGCATCACCTGGGAAGGCACCAACGGGGAGTTCAAGATGAC GGATCCCGACGAGGTGGCCCGGCGCTGGGGAGAGCGGAAGAGCAAACCCAACATGA ACTACGATAAGCTCAGCCGCGCCCTCCGTTACTACTATGACAAGAACATCATGACCAAG GTCCATGGGAAGCGCTACGCCTACAAGTTCGACTTCCACGGGATCGCCCAGGCCCTC CAGCCCCACCCCCCGGAGTCATCTCTGTACAAGTACCCCTCAGACCTCCCGTACATGG GCTCCTATCACGCCCACCCACAGAAGATGAACTTTGTGGCGCCCCACCCTCCAGCCCT CCCCGTGACATCTTCCAGTTTTTTTGCTGCCCCAAACCCATACTGGAATTCACCAACTG GGGGTATATACCCCAACACTAGGCTCCCCACCAGCCATATGCCTTCTCATCTGGGCAC TTACTAC (SEQ ID NO:174; NM_001136154), or a nucleic acid sequence that hybridizes to a nucleic acid sequence consisting of SEQ ID NO:174 under stringent hybridization conditions.

In some embodiments, PGC1a comprises the amino acid sequence: MAWDMCNQDSESVWSDIECAALVGEDQPLCPDLPELDLSELDVNDLDTDSFLGGLKWCS DQSEIISNQYNNEPSNIFEKIDEENEANLLAVLTETLDSLPVDEDGLPSFDALTDGDVTTDNE ASPSSMPDGTPPPQEAEEPSLLKKLLLAPANTQLSYNECSGLSTQNHANHNHRIRTNPAIV KTENSWSNKAKSICQQQKPQRRPCSELLKYLTTNDDPPHTKPTENRNSSRDKCTSKKKSH TQSQSQHLQAKPTTLSLPLTPESPNDPKGSPFENKTIERTLSVELSGTAGLTPPTTPPHKAN QDNPFRASPKLKSSCKTVVPPPSKKPRYSESSGTQGNNSTKKGPEQSELYAQLSKSSVLT GGHEERKTKRPSLRLFGDHDYCQSINSKTEILINISQELQDSRQLENKDVSSDWQGQICSST DSDQCYLRETLEASKQVSPCSTRKQLQDQEIRAELNKHFGHPSQAVFDDEADKTSELRDS DFSNEQFSKLPMFINSGLAMDGLFDDSEDESDKLSYPWDGTQSYSLFNVSPSCSSFNSPC RDSVSPPKSLFSQRPQRMRSRSRSFSRHRSCSRSPYSRSRSRSPGSRSSSRSCYYYESS HYRHRTHRNSPLYVRSRSRSPYSRRPRYDSYEEYQHERLKREEYRREYEKRESERAKQR ERQRQKAIEERRVIYVGKIRPDTTRTELRDRFEVFGEIEECTVNLRDDGDSYGFITYRYTCD AFAALENGYTLRRSNETDFELYFCGRKQFFKSNYADLDSNSDDFDPASTKSKYDSLDFDSL LKEAQRSLRR (SEQ ID NO:175; NP_037393), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:175.

In some embodiments, the nucleic acid sequence encoding PGC1a comprises the nucleic acid sequence: ATGGCGTGGGACATGTGCAACCAGGACTCTGAGTCTGTATGGAGTGACATCGAGTGTG CTGCTCTGGTTGGTGAAGACCAGCCTCTTTGCCCAGATCTTCCTGAACTTGATCTTTCT GAACTAGATGTGAACGACTTGGATACAGACAGCTTTCTGGGTGGACTCAAGTGGTGCA GTGACCAATCAGAAATAATATCCAATCAGTACAACAATGAGCCTTCAAACATATTTGAGA AGATAGATGAAGAGAATGAGGCAAACTTGCTAGCAGTCCTCACAGAGACACTAGACAG TCTCCCTGTGGATGAAGACGGATTGCCCTCATTTGATGCGCTGACAGATGGAGACGTG ACCACTGACAATGAGGCTAGTCCTTCCTCCATGCCTGACGGCACCCCTCCACCCCAGG AGGCAGAAGAGCCGTCTCTACTTAAGAAGCTCTTACTGGCACCAGCCAACACTCAGCT AAGTTATAATGAATGCAGTGGTCTCAGTACCCAGAACCATGCAAATCACAATCACAGGA TCAGAACAAACCCTGCAATTGTTAAGACTGAGAATTCATGGAGCAATAAAGCGAAGAGT ATTTGTCAACAGCAAAAGCCACAAAGACGTCCCTGCTCGGAGCTTCTCAAATATCTGAC CACAAACGATGACCCTCCTCACACCAAACCCACAGAGAACAGAAACAGCAGCAGAGAC AAATGCACCTCCAAAAAGAAGTCCCACACACAGTCGCAGTCACAACACTTACAAGCCAA ACCAACAACTTTATCTCTTCCTCTGACCCCAGAGTCACCAAATGACCCCAAGGGTTCCC CATTTGAGAACAAGACTATTGAACGCACCTTAAGTGTGGAACTCTCTGGAACTGCAGGC CTAACTCCACCCACCACTCCTCCTCATAAAGCCAACCAAGATAACCCTTTTAGGGCTTC TCCAAAGCTGAAGTCCTCTTGCAAGACTGTGGTGCCACCACCATCAAAGAAGCCCAGG TACAGTGAGTCTTCTGGTACACAAGGCAATAACTCCACCAAGAAAGGGCCGGAGCAAT CCGAGTTGTATGCACAACTCAGCAAGTCCTCAGTCCTCACTGGTGGACACGAGGAAAG GAAGACCAAGCGGCCCAGTCTGCGGCTGTTTGGTGACCATGACTATTGCCAGTCAATT AATTCCAAAACGGAAATACTCATTAATATATCACAGGAGCTCCAAGACTCTAGACAACTA GAAAATAAAGATGTCTCCTCTGATTGGCAGGGGCAGATTTGTTCTTCCACAGATTCAGA CCAGTGCTACCTGAGAGAGACTTTGGAGGCAAGCAAGCAGGTCTCTCCTTGCAGCACA AGAAAACAGCTCCAAGACCAGGAAATCCGAGCCGAGCTGAACAAGCACTTCGGTCATC CCAGTCAAGCTGTTTTTGACGACGAAGCAGACAAGACCAGTGAACTGAGGGACAGTGA TTTCAGTAATGAACAATTCTCCAAACTACCTATGTTTATAAATTCAGGACTAGCCATGGA TGGCCTGTTTGATGACAGCGAAGATGAAAGTGATAAACTGAGCTACCCTTGGGATGGC ACACAATCCTATTCATTGTTCAATGTGTCTCCTTCTTGTTCTTCTTTTAACTCTCCATGTA GAGATTCTGTGTCACCACCCAAATCCTTATTTTCTCAAAGACCCCAAAGGATGCGCTCT CGTTCAAGGTCCTTTTCTCGACACAGGTCGTGTTCCCGATCACCATATTCCAGGTCAAG ATCAAGGTCTCCAGGCAGTAGATCCTCTTCAAGATCCTGCTATTACTATGAGTCAAGCC ACTACAGACACCGCACGCACCGAAATTCTCCCTTGTATGTGAGATCACGTTCAAGATCG CCCTACAGCCGTCGGCCCAGGTATGACAGCTACGAGGAATATCAGCACGAGAGGCTG AAGAGGGAAGAATATCGCAGAGAGTATGAGAAGCGAGAGTCTGAGAGGGCCAAGCAA AGGGAGAGGCAGAGGCAGAAGGCAATTGAAGAGCGCCGTGTGATTTATGTCGGTAAA ATCAGACCTGACACAACACGGACAGAACTGAGGGACCGTTTTGAAGTTTTTGGTGAAAT TGAGGAGTGCACAGTAAATCTGCGGGATGATGGAGACAGCTATGGTTTCATTACCTAC CGTTATACCTGTGATGCTTTTGCTGCTCTTGAAAATGGATACACTTTGCGCAGGTCAAA CGAAACTGACTTTGAGCTGTACTTTTGTGGACGCAAGCAATTTTTCAAGTCTAACTATGC AGACCTAGATTCAAACTCAGATGACTTTGACCCTGCTTCCACCAAGAGCAAGTATGACT CTCTGGATTTTGATAGTTTACTGAAAGAAGCTCAGAGAAGCTTGCGCAGG (SEQ ID NO:176; NM_013261), or a nucleic acid sequence that hybridizes to a nucleic acid sequence consisting of SEQ ID NO:176 under stringent hybridization conditions.

In some embodiments, Osterix (SP7) comprises the amino acid sequence: MASSLLEEEVHYGSSPLAMLTAACSKFGGSSPLRDSTTLGKAGTKKPYSVGSDLSASKTM GDAYPAPFTSTNGLLSPAGSPPAPTSGYANDYPPFSHSFPGPTGTQDPGLLVPKGHSSSD CLPSVYTSLDMTHPYGSWYKAGIHAGISPGPGNTPTPWWDMHPGGNWLGGGQGQGDGL QGTLPTGPAQPPLNPQLPTYPSDFAPLNPAPYPAPHLLQPGPQHVLPQDVYKPKAVGNSG QLEGSGGAKPPRGASTGGSGGYGGSGAGRSSCDCPNCQELERLGAAAAGLRKKPIHSCH IPGCGKVYGKASHLKAHLRWHTGERPFVCNWLFCGKRFTRSDELERHVRTHTREKKFTCL LCSKRFTRSDHLSKHQRTHGEPGPGPPPSGPKELGEGRSTGEEEASQTPRPSASPATPEK APGGSPEQSNLLEI (SEQ ID NO:177; NP_690599), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:177.

In some embodiments, the nucleic acid sequence encoding Osterix comprises the nucleic acid sequence: ATGGCGTCCTCCCTGCTTGAGGAGGAAGTTCACTATGGCTCCAGTCCCCTGGCCATGC TGACGGCAGCGTGCAGCAAATTTGGTGGCTCTAGCCCTCTGCGGGACTCAACAACTCT GGGCAAAGCAGGCACAAAGAAGCCGTACTCTGTGGGCAGTGACCTTTCAGCCTCCAAA ACCATGGGGGATGCTTATCCAGCCCCCTTTACAAGCACTAATGGGCTCCTTTCACCTG CAGGCAGTCCTCCAGCACCCACCTCAGGCTATGCTAATGATTACCCTCCCTTTTCCCAC TCATTCCCTGGGCCCACAGGCACCCAGGACCCTGGGCTACTAGTGCCCAAGGGGCAC AGCTCTTCTGACTGTCTGCCCAGTGTCTACACCTCTCTGGACATGACACACCCCTATGG CTCCTGGTACAAGGCAGGCATCCATGCAGGCATTTCACCAGGCCCAGGCAACACTCCT ACTCCATGGTGGGATATGCACCCTGGAGGCAACTGGCTAGGTGGTGGGCAGGGCCAG GGTGATGGGCTGCAAGGGACACTGCCCACAGGTCCAGCTCAGCCTCCACTGAACCCC CAGCTGCCCACCTACCCATCTGACTTTGCTCCCCTTAATCCAGCCCCCTACCCAGCTC CCCACCTCTTGCAACCAGGGCCCCAGCATGTCTTGCCCCAAGATGTCTATAAACCCAA GGCAGTGGGAAATAGTGGGCAGCTAGAAGGGAGTGGTGGAGCCAAACCCCCACGGG GTGCAAGCACTGGGGGTAGTGGTGGATATGGGGGCAGTGGGGCAGGGCGCTCCTCC TGCGACTGCCCTAATTGCCAGGAGCTAGAGCGGCTGGGAGCAGCAGCGGCTGGGCTG CGGAAGAAGCCCATCCACAGCTGCCACATCCCTGGCTGCGGCAAGGTGTATGGCAAG GCTTCGCACCTGAAGGCCCACTTGCGCTGGCACACAGGCGAGAGGCCCTTCGTCTGC AACTGGCTCTTCTGCGGCAAGAGGTTCACTCGTTCGGATGAGCTGGAGCGTCATGTGC GCACTCACACCCGGGAGAAGAAGTTCACCTGCCTGCTCTGCTCCAAGCGCTTTACCCG AAGTGACCACCTGAGCAAACACCAGCGCACCCACGGAGAACCAGGCCCGGGTCCCCC TCCCAGTGGCCCCAAGGAGCTGGGGGAGGGCCGCAGCACGGGGGAAGAGGAGGCCA GTCAGACGCCCCGACCTTCTGCCTCGCCAGCAACCCCAGAGAAAGCCCCTGGAGGCA GCCCTGAGCAGAGCAACTTGCTGGAGATC (SEQ ID NO:178; NM_152860), or a nucleic acid sequence that hybridizes to a nucleic acid sequence consisting of SEQ ID NO:178 under stringent hybridization conditions.

In some embodiments, myocyte enhancer factor 2C (MEF2C) comprises the amino acid sequence: MGRKKIQITRIMDERNRQVTFTKRKFGLMKKAYELSVLCDCEIALIIFNSTNKLFQYASTDMD KVLLKYTEYNEPHESRTNSDIVETLRKKGLNGCDSPDPDADDSVGHSPESEDKYRKINEDI DLMISRQRLCAVPPPNFEMPVSIPVSSHNSLVYSNPVSSLGNPNLLPLAHPSLQRNSMSPG VTHRPPSAGNTGGLMGGDLTSGAGTSAGNGYGNPRNSPGLLVSPGNLNKNMQAKSPPP MNLGMNNRKPDLRVLIPPGSKNTMPSVSEDVDLLLNQRINNSQSAQSLATPVVSVATPTLP GQGMGGYPSAISTTYGTEYSLSSADLSSLSGFNTASALHLGSVTGWQQQHLHNMPPSALS QLGACTSTHLSQSSNLSLPSTQSLNIKSEPVSPPRDRTTTPSRYPQHTRHEAGRSPVDSLS SCSSSYDGSDREDHRNEFHSPIGLTRPSPDERESPSVKRMRLSEGWAT (SEQ ID NO:179; NP_002388), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:179.

In some embodiments, the nucleic acid sequence encoding MEF2C comprises the nucleic acid sequence: ATGGGGAGAAAAAAGATTCAGATTACGAGGATTATGGATGAACGTAACAGACAGGTGA CATTTACAAAGAGGAAATTTGGGTTGATGAAGAAGGCTTATGAGCTGAGCGTGCTGTGT GACTGTGAGATTGCGCTGATCATCTTCAACAGCACCAACAAGCTGTTCCAGTATGCCAG CACCGACATGGACAAAGTGCTTCTCAAGTACACGGAGTACAACGAGCCGCATGAGAGC CGGACAAACTCAGACATCGTGGAGACGTTGAGAAAGAAGGGCCTTAATGGCTGTGACA GCCCAGACCCCGATGCGGACGATTCCGTAGGTCACAGCCCTGAGTCTGAGGACAAGT ACAGGAAAATTAACGAAGATATTGATCTAATGATCAGCAGGCAAAGATTGTGTGCTGTT CCACCTCCCAACTTCGAGATGCCAGTCTCCATCCCAGTGTCCAGCCACAACAGTTTGG TGTACAGCAACCCTGTCAGCTCACTGGGAAACCCCAACCTATTGCCACTGGCTCACCC TTCTCTGCAGAGGAATAGTATGTCTCCTGGTGTAACACATCGACCTCCAAGTGCAGGTA ACACAGGTGGTCTGATGGGTGGAGACCTCACGTCTGGTGCAGGCACCAGTGCAGGGA ACGGGTATGGCAATCCCCGAAACTCACCAGGTCTGCTGGTCTCACCTGGTAACTTGAA CAAGAATATGCAAGCAAAATCTCCTCCCCCAATGAATTTAGGAATGAATAACCGTAAAC CAGATCTCCGAGTTCTTATTCCACCAGGCAGCAAGAATACGATGCCATCAGTGTCTGAG GATGTCGACCTGCTTTTGAATCAAAGGATAAATAACTCCCAGTCGGCTCAGTCATTGGC TACCCCAGTGGTTTCCGTAGCAACTCCTACTTTACCAGGACAAGGAATGGGAGGATAT CCATCAGCCATTTCAACAACATATGGTACCGAGTACTCTCTGAGTAGTGCAGACCTGTC ATCTCTGTCTGGGTTTAACACCGCCAGCGCTCTTCACCTTGGTTCAGTAACTGGCTGGC AACAGCAACACCTACATAACATGCCACCATCTGCCCTCAGTCAGTTGGGAGCTTGCACT AGCACTCATTTATCTCAGAGTTCAAATCTCTCCCTGCCTTCTACTCAAAGCCTCAACATC AAGTCAGAACCTGTTTCTCCTCCTAGAGACCGTACCACCACCCCTTCGAGATACCCACA ACACACGCGCCACGAGGCGGGGAGATCTCCTGTTGACAGCTTGAGCAGCTGTAGCAG TTCGTACGACGGGAGCGACCGAGAGGATCACCGGAACGAATTCCACTCCCCCATTGG ACTCACCAGACCTTCGCCGGACGAAAGGGAAAGTCCCTCAGTCAAGCGCATGCGACTT TCTGAAGGATGGGCAACA (SEQ ID NO:180; NM_002397), or a nucleic acid sequence that hybridizes to a nucleic acid sequence consisting of SEQ ID NO:180 under stringent hybridization conditions.

In some embodiments, Mohawk (MKX) comprises the amino acid sequence: MNTIVFNKLSSAVLFEDGGASERERGGRPYSGVLDSPHARPEVGIPDGPPLKDNLGLRHR RTGARQNGGKVRHKRQALQDMARPLKQWLYKHRDNPYPTKTEKILLALGSQMTLVQVSN WFANARRRLKNTVRQPDLSWALRIKLYNKYVQGNAERLSVSSDDSCSEDGENPPRTHMN EGGYNTPVHHPVIKSENSVIKAGVRPESRASEDYVAPPKYKSSLLNRYLNDSLRHVMATNT TMMGKTRQRNHSGSFSSNEFEEELVSPSSSETEGNFVYRTDTLENGSNKGESAANRKGP SKDDTYWKEINAAMALTNLAQGKDKLQGTTSCIIQKSSHIAEVKTVKVPLVQQF (SEQ ID NO:181; NP_775847), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:181.

In some embodiments, the nucleic acid sequence encoding MKX comprises the nucleic acid sequence: ATGAACACCATCGTCTTCAACAAGCTCAGCTCTGCGGTGCTGTTTGAGGACGGAGGCG CCTCGGAGCGGGAGCGGGGTGGCCGGCCCTACAGCGGTGTCCTGGACAGTCCTCAC GCCCGCCCCGAGGTGGGCATTCCCGACGGCCCGCCCCTCAAGGACAACCTCGGCCT GAGACACCGGAGGACCGGCGCCCGGCAGAATGGCGGGAAGGTGAGGCACAAGCGGC AGGCCCTGCAAGACATGGCGCGACCCCTCAAGCAGTGGCTTTACAAGCACCGTGACA ACCCGTACCCCACCAAGACCGAGAAGATACTCTTGGCCCTCGGCTCGCAGATGACGCT AGTGCAGGTGTCAAATTGGTTTGCTAATGCAAGACGTCGGCTTAAGAATACCGTTCGAC AGCCAGATTTAAGCTGGGCTTTGAGAATAAAGTTATACAACAAGTATGTTCAAGGCAAT GCTGAACGGCTTAGCGTAAGCAGTGATGACTCATGTTCTGAAGATGGAGAAAATCCTC CAAGAACCCACATGAACGAAGGGGGCTATAATACCCCAGTTCACCATCCTGTGATTAAA AGTGAGAATTCGGTCATCAAAGCGGGAGTGAGGCCAGAGTCACGGGCCAGTGAGGAC TACGTGGCACCCCCCAAATACAAGAGCAGCTTGTTGAACCGTTACCTTAATGACTCTTT GAGACATGTCATGGCCACGAACACTACCATGATGGGAAAAACAAGGCAAAGAAACCAC TCGGGATCTTTTAGCTCCAATGAATTTGAGGAAGAATTAGTGTCTCCATCGTCATCAGA AACTGAAGGCAACTTTGTCTATCGCACAGACACTCTGGAAAATGGATCCAATAAGGGTG AAAGCGCAGCTAACAGAAAAGGACCAAGCAAGGATGACACGTATTGGAAGGAGATCAA CGCAGCTATGGCCTTAACAAATCTTGCACAGGGAAAGGACAAACTGCAGGGAACTACC AGCTGCATCATCCAGAAGTCGTCCCATATAGCAGAAGTAAAGACTGTCAAAGTGCCGC TGGTGCAGCAGTTT (SEQ ID NO:182; NM_173576), or a nucleic acid sequence that hybridizes to a nucleic acid sequence consisting of SEQ ID NO:182 under stringent hybridization conditions.

In some embodiments, Iroquois homeobox 1 (IRX1) comprises the amino acid sequence: MSFPQLGYPQYLSAAGPGAYGGERPGVLAAAAAAAAAASSGRPGAAELGGGAGAAAVTS VLGMYAAAGPYAGAPNYSAFLPYAADLSLFSQMGSQYELKDNPGVHPATFAAHTAPAYYP YGQFQYGDPGRPKNATRESTSTLKAWLNEHRKNPYPTKGEKIMLAIITKMTLTQVSTWFAN ARRRLKKENKVTWGARSKDQEDGALFGSDTEGDPEKAEDDEEIDLESIDIDKIDEHDGDQS NEDDEDKAEAPHAPAAPSALARDQGSPLAAADVLKPQDSPLGLAKEAPEPGSTRLLSPGA AAGGLQGAPHGKPKIWSLAETATSPDGAPKASPPPPAGHPGAHGPSAGAPLQHPAFLPSH GLYTCHIGKFSNWTNSAFLAQGSLLNMRSFLGVGAPHAAPHGPHLPAPPPPQPPVAIAPGA LNGDKASVRSSPTLPERDLVPRPDSPAQQLKSPFQPVRDNSLAPQEGTPRILAALPSA (SEQ ID NO:183; NP_077313), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:183.

In some embodiments, the nucleic acid sequence encoding IRX1 comprises the nucleic acid sequence: ATGTCCTTCCCGCAGCTGGGCTACCCGCAGTACCTGAGCGCCGCGGGGCCGGGCGC CTACGGCGGCGAGCGCCCGGGGGTGCTGGCCGCGGCCGCTGCGGCGGCTGCCGCC GCCTCGTCGGGCCGACCGGGGGCCGCGGAGCTGGGCGGCGGGGCAGGCGCGGCTG CAGTCACCTCGGTGCTGGGCATGTACGCGGCGGCGGGGCCGTACGCGGGCGCGCCC AACTACAGCGCCTTCCTGCCCTACGCCGCGGATCTCAGCCTCTTCTCGCAGATGGGCT CGCAGTATGAACTGAAGGACAACCCTGGGGTGCACCCCGCCACCTTCGCAGCCCACA CGGCGCCGGCTTATTACCCCTACGGCCAGTTCCAATACGGGGACCCCGGGCGGCCCA AGAACGCCACCCGCGAGAGCACCAGCACGCTCAAGGCCTGGCTCAACGAGCACCGCA AGAATCCCTACCCCACCAAGGGCGAGAAGATCATGCTGGCCATCATCACCAAGATGAC CCTCACGCAGGTCTCCACCTGGTTCGCCAACGCGCGCCGGCGCCTCAAGAAGGAGAA CAAGGTGACATGGGGAGCGCGCAGCAAGGACCAGGAAGATGGAGCGCTCTTCGGCA GCGACACCGAGGGCGACCCGGAGAAGGCCGAGGACGACGAGGAGATCGACCTGGAA AGCATCGACATTGACAAGATCGACGAGCACGATGGCGACCAGAGCAACGAGGATGAC GAGGACAAGGCCGAGGCTCCGCACGCGCCCGCAGCCCCTTCTGCTCTTGCCCGGGA CCAAGGCTCGCCGCTGGCAGCAGCCGACGTTCTCAAGCCCCAGGACTCGCCCTTGGG CCTGGCAAAGGAGGCCCCAGAGCCGGGCAGCACGCGCCTGCTGAGCCCCGGCGCTG CAGCGGGCGGCCTGCAGGGTGCGCCGCACGGCAAGCCCAAGATCTGGTCGCTGGCG GAGACAGCCACGAGCCCCGACGGTGCGCCCAAGGCTTCGCCACCACCACCCGCGGG CCACCCCGGCGCGCACGGGCCCTCCGCCGGGGCGCCGCTGCAACACCCCGCCTTCC TGCCTAGCCACGGACTGTACACCTGCCACATCGGCAAGTTCTCCAACTGGACCAACAG CGCATTCCTCGCACAGGGCTCCCTGCTCAACATGCGCTCCTTCCTGGGCGTTGGCGCT CCCCACGCCGCGCCCCATGGCCCTCACCTTCCTGCACCTCCACCACCGCAGCCGCCG GTCGCTATTGCCCCGGGGGCACTCAATGGAGACAAGGCCTCGGTCCGCAGCAGCCCC ACGCTCCCAGAGAGAGACCTCGTCCCCAGGCCAGATTCGCCGGCACAGCAGTTAAAG TCGCCCTTCCAGCCGGTACGCGACAACTCTCTGGCCCCGCAGGAGGGAACGCCGCGG ATCCTAGCAGCCCTCCCGTCCGCC (SEQ ID NO:184; NM_024337), or a nucleic acid sequence that hybridizes to a nucleic acid sequence consisting of SEQ ID NO:184 under stringent hybridization conditions.

In some embodiments, iroquois homeobox 2 (IRX2) comprises the amino acid sequence: MSYPQGYLYQAPGSLALYSCPAYGASALAAPRSEELARSASGSAFSPYPGSAAFTAQAAT GFGSPLQYSADAAAAAAGFPSYMGAPYDAHTTGMTGAISYHPYGSAAYPYQLNDPAYRKN ATRDATATLKAWLNEHRKNPYPTKGEKIMLAIITKMTLTQVSTWFANARRRLKKENKMTWA PRNKSEDEDEDEGDATRSKDESPDKAQEGTETSAEDEGISLHVDSLTDHSCSAESDGEKL PCRAGDPLCESGSECKDKYDDLEDDEDDDEEGERGLAPPKPVTSSPLTGLEAPLLSPPPE AAPRGGRKTPQGSRTSPGAPPPASKPKLWSLAEIATSDLKQPSLGPGCGPPGLPAAAAPA STGAPPGGSPYPASPLLGRPLYYTSPFYGNYTNYGNLNAALQGQGLLRYNSAAAAPGEAL HTAPKAASDAGKAGAHPLESHYRSPGGGYEPKKDASEGCTVVGGGVQPYL (SEQ ID NO:185; NP_150366), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:185.

In some embodiments, the nucleic acid sequence encoding IRX2 comprises the nucleic acid sequence: ATGTCCTACCCGCAGGGCTACCTGTACCAGGCGCCCGGCTCGCTGGCGCTCTACTCG TGCCCGGCCTACGGCGCGTCGGCTTTGGCGGCTCCGCGCAGCGAGGAGCTGGCGCG CTCGGCGTCGGGCTCGGCGTTCAGCCCCTACCCGGGCTCGGCGGCCTTCACGGCGC AGGCGGCCACCGGCTTCGGGAGCCCGCTGCAGTACTCGGCCGACGCCGCCGCCGCC GCCGCCGGCTTCCCGTCCTACATGGGCGCACCCTACGACGCGCACACCACCGGCATG ACCGGCGCCATCAGCTACCACCCGTACGGCAGCGCGGCCTACCCGTACCAGCTCAAC GACCCCGCGTACCGCAAGAACGCCACGCGGGACGCCACGGCCACTCTCAAGGCCTG GCTCAACGAGCACCGCAAGAACCCCTACCCCACCAAGGGCGAGAAGATCATGCTAGC CATCATCACCAAGATGACCCTCACCCAGGTCTCCACCTGGTTCGCCAACGCGCGCCGG CGCCTCAAGAAGGAGAACAAGATGACCTGGGCCCCGAGAAACAAAAGCGAAGATGAG GACGAGGACGAGGGCGACGCTACCAGAAGCAAGGACGAGAGTCCCGACAAGGCGCA GGAGGGCACGGAGACCTCGGCAGAGGACGAAGGGATCAGCCTGCACGTGGACTCGC TCACGGATCACTCGTGCTCGGCCGAGTCGGACGGGGAGAAGCTTCCGTGCCGCGCCG GGGACCCCCTGTGCGAATCGGGCTCGGAGTGCAAGGACAAGTATGACGACCTGGAGG ACGACGAGGACGACGACGAGGAGGGCGAGCGGGGCCTGGCGCCGCCCAAGCCCGT GACCTCGTCGCCGCTTACCGGCTTGGAGGCGCCGCTGCTGAGCCCCCCGCCCGAGG CCGCGCCCCGCGGTGGCCGCAAGACGCCCCAGGGCAGCCGGACGTCTCCGGGCGC GCCGCCCCCCGCCAGCAAGCCCAAGCTGTGGTCGCTGGCCGAGATCGCCACGTCGG ACCTCAAGCAGCCGAGCCTGGGCCCGGGCTGCGGGCCACCGGGGCTGCCCGCGGCC GCCGCGCCGGCCTCAACCGGGGCACCGCCAGGAGGCTCGCCCTACCCTGCCTCGCC GCTGCTGGGCCGCCCCCTCTACTACACGTCGCCCTTCTACGGCAACTACACAAACTAC GGGAACTTGAACGCGGCGCTGCAGGGCCAGGGTCTCCTGCGGTACAACTCTGCGGCC GCGGCCCCCGGCGAGGCCCTGCACACCGCGCCAAAGGCGGCCAGCGACGCGGGCA AGGCGGGCGCGCACCCGCTCGAGTCCCACTACCGGTCCCCGGGCGGCGGCTACGAG CCCAAGAAAGATGCCAGCGAGGGCTGCACCGTGGTTGGCGGGGGCGTCCAGCCCTA CCTA (SEQ ID NO:186; NM_033267), or a nucleic acid sequence that hybridizes to a nucleic acid sequence consisting of SEQ ID NO:186 under stringent hybridization conditions.

In some embodiments, iroquois homeobox 3 (IRX3) comprises the amino acid sequence: MSFPQLGYQYIRPLYPSERPGAAGGSGGSAGARGGLGAGASELNASGSLSNVLSSVYGA PYAAAAAAAAAQGYGAFLPYAAELPIFPQLGAQYELKDSPGVQHPAAAAAFPHPHPAFYPY GQYQFGDPSRPKNATRESTSTLKAWLNEHRKNPYPTKGEKIMLAIITKMTLTQVSTWFANA RRRLKKENKMTWAPRSRTDEEGNAYGSEREEEDEEEDEEDGKRELELEEEELGGEEEDT GGEGLADDDEDEEIDLENLDGAATEPELSLAGAARRDGDLGLGPISDSKNSDSEDSSEGLE DRPLPVLSLAPAPPPVAVASPSLPSPPVSLDPCAPAPAPASALQKPKIWSLAETATSPDNPR RSPPGAGGSPPGAAVAPSALQLSPAAAAAAAHRLVSAPLGKFPAWTNRPFPGPPPGPRP HPLSLLGSAPPHLLGLPGAAGHPAAAAAFARPAEPEGGTDRCSALEVEKKLLKTAFQPVPR RPQNHLDAALVLSALSSS (SEQ ID NO:187; NP_077312), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:187.

In some embodiments, the nucleic acid sequence encoding IRX3 comprises the nucleic acid sequence ATGTCCTTCCCCCAGCTGGGATACCAATACATCCGCCCGCTTTACCCGTCCGAGCGCC CGGGGGCCGCTGGCGGCAGCGGCGGCAGCGCGGGGGCCCGGGGCGGCCTGGGTG CCGGAGCCTCGGAGCTGAACGCCTCGGGGTCCCTGTCCAACGTGCTCTCGTCCGTGT ACGGGGCGCCCTACGCCGCGGCCGCTGCGGCCGCCGCCGCCCAAGGCTACGGCGC CTTCCTGCCCTACGCCGCGGAGCTGCCCATCTTCCCGCAGCTGGGCGCGCAGTATGA GCTGAAGGACAGCCCCGGGGTGCAGCATCCGGCCGCGGCTGCCGCGTTTCCGCACC CGCACCCCGCCTTCTACCCGTATGGCCAGTACCAGTTCGGGGACCCGTCCCGTCCCA AGAACGCCACCAGGGAGAGCACCAGCACGCTGAAGGCCTGGCTCAACGAGCACCGCA AGAACCCCTACCCCACCAAGGGCGAGAAGATCATGCTGGCCATCATCACCAAGATGAC CCTCACCCAGGTGTCCACCTGGTTCGCCAACGCGCGCCGGCGCCTCAAGAAGGAGAA TAAGATGACTTGGGCGCCTCGCAGCCGCACTGACGAGGAGGGAAACGCTTATGGGAG CGAGCGCGAGGAGGAAGACGAAGAGGAGGACGAGGAGGACGGCAAACGCGAGCTAG AGCTGGAGGAGGAGGAGCTCGGGGGGGAGGAGGAGGACACGGGGGGCGAGGGCCT GGCTGACGACGACGAGGACGAGGAGATCGATTTGGAGAACTTAGACGGCGCGGCCAC CGAGCCTGAGCTGTCCCTGGCTGGGGCGGCGCGCAGGGATGGCGACCTAGGCCTGG GACCCATTTCGGACTCCAAAAATAGCGACTCGGAAGATAGCTCTGAGGGCTTAGAGGA CCGGCCACTACCGGTCCTGAGTCTGGCTCCAGCGCCACCACCAGTGGCCGTGGCCTC GCCGTCTCTGCCGTCGCCCCCCGTGAGCCTGGACCCCTGCGCTCCCGCACCAGCCCC CGCCTCCGCCCTGCAGAAGCCCAAGATCTGGTCCCTCGCGGAGACTGCCACAAGCCC GGACAACCCGCGCCGCTCGCCTCCCGGCGCGGGGGGGTCTCCACCGGGGGCAGCG GTCGCGCCTTCCGCCCTGCAGCTCTCTCCGGCCGCCGCCGCCGCCGCCGCTCACAGA CTGGTCTCAGCGCCGCTGGGCAAGTTCCCGGCTTGGACCAACCGGCCGTTTCCAGGC CCACCGCCCGGCCCCCGCCCGCACCCGCTCTCCCTGCTGGGCTCTGCCCCTCCGCAC CTGCTGGGACTTCCCGGAGCCGCGGGCCACCCGGCTGCCGCCGCCGCCTTCGCTCG GCCAGCGGAGCCCGAAGGCGGAACAGATCGCTGTAGTGCCTTGGAAGTGGAGAAAAA GTTACTCAAGACAGCTTTCCAGCCCGTGCCCAGGCGGCCCCAGAACCATCTGGACGC CGCCCTGGTCTTATCGGCTCTCTCCTCATCC (SEQ ID NO:188; NM_024336), or a nucleic acid sequence that hybridizes to a nucleic acid sequence consisting of SEQ ID NO:188 under stringent hybridization conditions.

In some embodiments, iroquois homeobox 4 (IRX4) comprises the amino acid sequence: MSYPQFGYPYSSAPQFLMATNSLSTCCESGGRTLADSGPAASAQAPVYCPVYESRLLATA RHELNSAAALGVYGGPYGGSQGYGNYVTYGSEASAFYSLNSFDSKDGSGSAHGGLAPAT AAYYPYEPALGQYPYDRYGTMDSGTRRKNATRETTSTLKAWLQEHRKNPYPTKGEKIMLAI ITKMTLTQVSTWFANARRRLKKENKMTWPPRNKCADEKRPYAEGEEEEGGEEEAREEPLK SSKNAEPVGKEEKELELSDLDDFDPLEAEPPACELKPPFHSLDGGLERVPAAPDGPVKEAS GALRMSLAAGGGAALDEDLERARSCLRSAAAGPEPLPGAEGGPQVCEAKLGFVPAGASA GLEAKPRIWSLAHTATAAAAAATSLSQTEFPSCMLKRQGPAAPAAVSSAPATSPSVALPHS GALDRHQDSPVTSLRNWVDGVFHDPILRHSTLNQAWATAKGALLDPGPLGRSLGAGANVL TAPLARAFPPAVPQDAPAAGAARELLALPKAGGKPFCA (SEQ ID NO:189; NP_057442), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:189.

In some embodiments, the nucleic acid sequence encoding IRX4 comprises the nucleic acid sequence: ATGTCCTACCCGCAGTTTGGATACCCCTACTCCTCGGCTCCCCAGTTCTTGATGGCCA CCAACTCCCTGAGCACGTGCTGCGAGTCCGGAGGCCGCACGCTGGCGGACTCCGGG CCCGCCGCCTCGGCCCAGGCGCCGGTCTACTGCCCGGTCTACGAGAGCCGGCTGCT GGCCACCGCGCGCCACGAGCTCAACTCGGCCGCGGCGCTGGGCGTCTATGGGGGTC CCTATGGCGGATCGCAGGGCTATGGCAACTACGTGACCTACGGCTCGGAGGCGTCCG CCTTCTACTCGCTGAACAGCTTTGATTCCAAGGATGGTTCGGGATCTGCGCATGGGGG CCTGGCACCAGCCACTGCCGCCTACTACCCTTACGAGCCAGCTCTGGGCCAGTACCC CTATGACAGGTATGGAACCATGGACAGCGGCACGCGGCGCAAGAACGCCACGCGCGA GACCACCAGCACGCTCAAGGCCTGGCTGCAGGAGCACCGCAAGAACCCCTACCCCAC CAAGGGCGAGAAGATCATGCTGGCCATCATCACCAAGATGACCCTCACACAGGTCTCC ACCTGGTTCGCCAACGCGCGCCGGCGCCTCAAGAAGGAGAACAAGATGACGTGGCCG CCGCGGAACAAGTGCGCAGACGAGAAGCGGCCCTACGCGGAGGGCGAGGAGGAGGA GGGGGGCGAGGAGGAGGCGCGGGAGGAGCCCCTCAAGAGCTCCAAGAACGCAGAGC CCGTGGGCAAAGAGGAGAAGGAGCTGGAGCTTAGTGACTTGGACGACTTCGACCCGC TGGAAGCAGAGCCGCCGGCGTGCGAGCTGAAGCCGCCCTTCCACTCCCTGGACGGC GGTCTGGAGCGCGTCCCCGCCGCGCCCGACGGCCCGGTCAAGGAGGCCTCAGGCGC GCTCCGGATGTCTCTGGCCGCGGGTGGCGGAGCTGCTCTGGACGAGGACCTGGAGA GGGCCCGGAGCTGTCTCCGCAGCGCGGCGGCCGGGCCGGAGCCACTGCCGGGCGC AGAGGGCGGCCCTCAGGTCTGCGAGGCCAAGCTGGGGTTTGTGCCGGCGGGGGCGT CGGCAGGCCTGGAGGCTAAGCCGCGCATCTGGTCCCTGGCCCACACAGCCACCGCC GCCGCCGCCGCCGCCACCTCCCTGAGCCAGACTGAGTTTCCGTCGTGCATGCTCAAG CGCCAAGGTCCCGCGGCCCCTGCGGCTGTGTCCTCCGCGCCCGCCACGTCCCCGTCT GTGGCCCTTCCCCACTCTGGCGCCCTGGACAGGCACCAGGACTCCCCGGTAACCAGT CTCAGAAACTGGGTGGACGGGGTCTTCCACGACCCCATCCTCAGGCACAGCACTTTGA ACCAGGCCTGGGCCACCGCCAAGGGCGCCCTCCTGGACCCCGGGCCTCTGGGACGC TCGCTGGGGGCGGGCGCGAACGTGCTGACTGCACCCCTGGCCCGCGCCTTTCCGCC TGCCGTGCCCCAGGACGCCCCAGCTGCAGGCGCCGCCAGGGAGCTGCTCGCCCTGC CCAAGGCCGGCGGCAAACCCTTCTGCGCC (SEQ ID NO:190; NM_016358), or a nucleic acid sequence that hybridizes to a nucleic acid sequence consisting of SEQ ID NO:190 under stringent hybridization conditions.

In some embodiments, iroquois homeobox 5 (IRX5) comprises the amino acid sequence: MSYPQGYLYQPSASLALYSCPAYSTSVISGPRTDELGRSSSGSAFSPYAGSTAFTAPSPGY NSHLQYGADPAAAAAAAFSSYVGSPYDHTPGMAGSLGYHPYAAPLGSYPYGDPAYRKNA TRDATATLKAWLNEHRKNPYPTKGEKIMLAIITKMTLTQVSTWFANARRRLKKENKMTWTP RNRSEDEEEEENIDLEKNDEDEPQKPEDKGDPEGPEAGGAEQKAASGCERLQGPPTPAG KETEGSLSDSDFKEPPSEGRLDALQGPPRTGGPSPAGPAAARLAEDPAPHYPAGAPAPGP HPAAGEVPPGPGGPSVIHSPPPPPPPAVLAKPKLWSLAEIATSSDKVKDGGGGNEGSPCP PCPGPIAGQALGGSRASPAPAPSRSPSAQCPFPGGTVLSRPLYYTAPFYPGYTNYGSFGH LHGHPGPGPGPTTGPGSHFNGLNQTVLNRADALAKDPKMLRSQSQLDLCKDSPYELKKG MSDI (SEQ ID NO:191; NP_005844), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:191.

In some embodiments, the nucleic acid sequence encoding IRX5 comprises the nucleic acid sequence: ATGTCCTATCCGCAGGGCTACTTGTACCAGCCGTCCGCCTCGCTGGCGCTCTACTCGT GCCCGGCGTACAGCACCAGCGTCATTTCGGGGCCCCGCACGGATGAGCTCGGCCGCT CTTCTTCGGGCTCCGCGTTCTCGCCCTACGCTGGCTCGACTGCCTTCACGGCGCCCTC GCCGGGCTACAACTCGCACCTCCAGTACGGCGCCGACCCCGCGGCCGCCGCCGCCG CCGCCTTCTCCTCGTACGTGGGCTCTCCCTACGACCACACACCCGGCATGGCGGGCT CCTTGGGGTACCATCCTTACGCGGCGCCCCTGGGATCGTACCCTTACGGGGACCCAG CGTACCGGAAGAACGCCACAAGGGACGCCACGGCTACCCTCAAGGCCTGGCTCAACG AGCACCGCAAGAACCCCTACCCCACCAAGGGCGAGAAGATCATGCTGGCCATCATCAC CAAGATGACCCTCACCCAGGTGTCCACCTGGTTCGCCAACGCGCGCCGGCGCCTCAA GAAAGAGAATAAAATGACGTGGACGCCGCGGAACCGCAGCGAGGACGAGGAAGAGGA GGAGAACATTGACCTGGAGAAGAACGACGAGGACGAGCCCCAGAAGCCCGAGGACAA GGGCGACCCCGAGGGCCCCGAAGCAGGAGGAGCTGAGCAGAAGGCGGCTTCGGGCT GCGAACGGCTTCAGGGACCACCCACCCCTGCAGGCAAGGAGACGGAGGGCAGCCTC AGCGACTCGGATTTTAAGGAGCCGCCCTCGGAGGGCCGCCTCGACGCGCTGCAGGG CCCCCCCCGCACCGGCGGGCCCTCCCCGGCTGGGCCAGCGGCGGCGCGGCTGGCG GAGGACCCGGCCCCTCACTACCCCGCCGGAGCGCCGGCGCCCGGCCCGCATCCAGC CGCGGGCGAGGTGCCTCCGGGTCCCGGCGGGCCCTCGGTTATCCATTCGCCGCCTC CGCCGCCGCCTCCTGCGGTGCTCGCCAAGCCCAAACTGTGGTCTTTGGCAGAGATCG CCACATCGTCGGACAAGGTCAAGGACGGGGGCGGCGGGAACGAGGGCTCTCCATGC CCACCGTGTCCCGGGCCCATAGCCGGGCAAGCCCTAGGAGGCAGCCGGGCGTCGCC GGCCCCGGCGCCGTCACGCTCGCCCTCGGCGCAGTGTCCTTTTCCAGGCGGGACGG TGCTGTCCCGGCCTCTCTACTACACCGCGCCCTTCTATCCCGGCTACACGAACTATGG CTCCTTCGGACACCTTCATGGCCACCCGGGGCCCGGGCCAGGCCCCACAACCGGTCC GGGGTCTCATTTCAATGGATTAAACCAGACCGTGTTGAACCGAGCGGACGCTTTGGCT AAAGACCCGAAAATGTTGCGGAGCCAGTCTCAGCTAGACCTGTGCAAAGACTCTCCCT ATGAATTGAAGAAAGGTATGTCCGACATT (SEQ ID NO:192; NM_005853), or a nucleic acid sequence that hybridizes to a nucleic acid sequence consisting of SEQ ID NO:192 under stringent hybridization conditions.

In some embodiments, iroquois homeobox 6 (IRX6) comprises the amino acid sequence MSFPHFGHPYRGASQFLASASSSTTCCESTQRSVSDVASGSTPAPALCCAPYDSRLLGSA RPELGAALGIYGAPYAAAAAAQSYPGYLPYSPEPPSLYGALNPQYEFKEAAGSFTSSLAQP GAYYPYERTLGQYQYERYGAVELSGAGRRKNATRETTSTLKAWLNEHRKNPYPTKGEKIM LAIITKMTLTQVSTWFANARRRLKKENKMTWAPKNKGGEERKAEGGEEDSLGCLTADTKE VTASQEARGLRLSDLEDLEEEEEEEEEAEDEEVVATAGDRLTEFRKGAQSLPGPCAAARE GRLERRECGLAAPRFSFNDPSGSEEADFLSAETGSPRLTMHYPCLEKPRIWSLAHTATASA VEGAPPARPRPRSPECRMIPGQPPASARRLSVPRDSACDESSCIPKAFGNPKFALQGLPL NCAPCPRRSEPVVQCQYPSGAEAG (SEQ ID NO:193; NP_077311), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:193.

In some embodiments, the nucleic acid sequence encoding IRX6 comprises the nucleic acid sequence: ATGTCCTTCCCACACTTTGGACACCCGTACCGCGGCGCTTCCCAGTTTCTGGCGTCGG CAAGTTCCAGCACCACATGCTGCGAATCTACCCAACGCTCTGTCTCAGATGTGGCATC AGGCTCCACCCCAGCGCCCGCTCTCTGCTGCGCACCCTACGATAGTCGACTGCTGGG CAGTGCGCGACCGGAGCTGGGCGCCGCCTTGGGCATCTATGGAGCACCCTATGCGGC CGCTGCAGCTGCCCAGAGCTACCCTGGCTACCTGCCCTATAGCCCAGAGCCCCCCTC ACTGTATGGGGCACTGAATCCACAGTATGAATTTAAGGAGGCTGCAGGGAGTTTTACAT CCAGCCTGGCACAACCAGGAGCCTATTATCCCTATGAGCGGACTCTGGGGCAGTACCA ATATGAACGGTATGGCGCAGTGGAATTGAGTGGCGCCGGTCGCCGAAAGAACGCGAC CCGGGAGACCACCAGTACACTCAAGGCCTGGCTCAACGAGCACCGCAAAAACCCCTA CCCCACTAAGGGTGAGAAGATCATGCTGGCCATCATCACCAAGATGACCCTCACCCAG GTGTCCACCTGGTTCGCCAACGCACGCCGGCGCCTCAAGAAAGAGAACAAAATGACAT GGGCGCCCAAGAACAAAGGTGGGGAGGAGAGGAAGGCAGAGGGAGGAGAGGAGGAC TCACTAGGCTGCCTAACTGCTGACACCAAAGAAGTTACTGCTAGCCAGGAGGCCCGGG GGCTCCGGCTGAGTGACCTGGAAGACCTGGAGGAAGAGGAGGAGGAGGAGGAGGAA GCTGAAGACGAGGAGGTAGTGGCCACAGCTGGGGACAGGCTGACGGAGTTCCGAAAG GGCGCGCAGTCACTGCCTGGGCCGTGCGCTGCAGCTCGAGAGGGCCGATTGGAGCG CAGGGAGTGCGGCCTGGCTGCGCCCCGCTTCTCCTTCAATGACCCTTCCGGATCGGA AGAAGCTGACTTCCTCTCGGCGGAGACAGGCAGCCCTAGGTTGACCATGCACTACCCA TGCTTGGAGAAACCGCGCATCTGGTCTCTGGCGCACACCGCGACAGCCAGCGCTGTT GAAGGTGCACCCCCAGCCCGGCCTAGGCCACGAAGTCCTGAGTGCCGTATGATTCCT GGACAGCCTCCTGCCTCTGCCCGGCGACTCTCAGTCCCCAGAGACTCCGCGTGCGAC GAGTCTTCCTGCATACCCAAAGCCTTTGGAAACCCCAAGTTTGCCCTGCAGGGACTAC CGCTGAACTGTGCGCCGTGCCCGCGGAGGAGCGAGCCTGTAGTGCAGTGCCAGTACC CGTCTGGAGCAGAAGCAGGT (SEQ ID NO:194; NM_024335), or a nucleic acid sequence that hybridizes to a nucleic acid sequence consisting of SEQ ID NO:194 under stringent hybridization conditions.

In some embodiments, Scleraxis (SCX) comprises the amino acid sequence: MSFATLRPAPPGRYLYPEVSPLSEDEDRGSDSSGSDEKPCRVHAARCGLQGARRRAGGR RAGGGGPGGRPGREPRQRHTANARERDRTNSVNTAFTALRTLIPTEPADRKLSKIETLRLA SSYISHLGNVLLAGEACGDGQPCHSGPAFFHAARAGSPPPPPPPPPARDGENTQPKQICT FCLSNQRKLSKDRDRKTAIRS (SEQ ID NO:195; NP_001073983), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:195.

In some embodiments, the nucleic acid sequence encoding SCX comprises the nucleic acid sequence: ATGTCCTTCGCCACGCTGCGCCCGGCGCCGCCGGGCCGCTACCTGTACCCCGAGGTG AGCCCGCTGTCGGAGGACGAGGACCGCGGCAGCGACAGCTCGGGCTCCGACGAGAA ACCCTGTCGCGTGCACGCGGCGCGCTGCGGCCTCCAGGGCGCCCGGCGGAGGGCG GGGGGCCGGCGGGCCGGGGGCGGGGGGCCAGGGGGCCGGCCAGGCCGTGAGCCC CGGCAGCGGCACACGGCGAACGCGCGCGAGCGAGACCGCACCAACAGCGTGAACAC GGCCTTCACGGCGCTGCGCACGCTGATCCCCACCGAGCCCGCCGACCGCAAGCTCTC CAAGATTGAGACGCTGCGCCTGGCCTCCAGCTACATCTCGCACCTGGGCAACGTGCT GCTGGCGGGCGAGGCCTGCGGCGACGGACAGCCCTGCCACTCCGGGCCCGCCTTCT TCCACGCGGCGCGCGCCGGCAGCCCCCCGCCGCCGCCCCCGCCGCCTCCCGCCCG CGACGGCGAGAACACCCAGCCCAAACAGATCTGCACCTTCTGCCTCAGCAACCAGAGA AAGTTGAGCAAGGACCGCGACAGAAAGACAGCGATTCGCAGT (SEQ ID NO:196; NM_001080514), or a nucleic acid sequence that hybridizes to a nucleic acid sequence consisting of SEQ ID NO:196 under stringent hybridization conditions.

In some embodiments, runt-related transcription factor 1 (RUNX1) comprises the amino acid sequence: MASDSIFESFPSYPQCFMRECILGMNPSRDVHDASTSRRFTPPSTALSPGKMSEALPLGAP DAGAALAGKLRSGDRSMVEVLADHPGELVRTDSPNFLCSVLPTHWRCNKTLPIAFKVVALG DVPDGTLVTVMAGNDENYSAELRNATAAMKNQVARFNDLRFVGRSGRGKSFTLTITVFTN PPQVATYHRAIKITVDGPREPRRHRQKLDDQTKPGSLSFSERLSELEQLRRTAMRVSPHHP APTPNPRASLNHSTAFNPQPQSQMQDTRQIQPSPPWSYDQSYQYLGSIASPSVHPATPIS PGRASGMTTLSAELSSRLSTAPDLTAFSDPRQFPALPSISDPRMHYPGAFTYSPTPVTSGIG IGMSAMGSATRYHTYLPPPYPGSSQAQGGPFQASSPSYHLYYGASAGSYQFSMVGGERS PPRILPPCTNASTGSALLNPSLPNQSDVVEAEGSHSNSPTNMAPSARLEEAVWRPY (SEQ ID NO:197; NP_001745), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:197.

In some embodiments, the nucleic acid sequence encoding RUNX1 comprises the nucleic acid sequence: ATGGCTTCAGACAGCATATTTGAGTCATTTCCTTCGTACCCACAGTGCTTCATGAGAGA ATGCATACTTGGAATGAATCCTTCTAGAGACGTCCACGATGCCAGCACGAGCCGCCGC TTCACGCCGCCTTCCACCGCGCTGAGCCCAGGCAAGATGAGCGAGGCGTTGCCGCTG GGCGCCCCGGACGCCGGCGCTGCCCTGGCCGGCAAGCTGAGGAGCGGCGACCGCA GCATGGTGGAGGTGCTGGCCGACCACCCGGGCGAGCTGGTGCGCACCGACAGCCCC AACTTCCTCTGCTCCGTGCTGCCTACGCACTGGCGCTGCAACAAGACCCTGCCCATCG CTTTCAAGGTGGTGGCCCTAGGGGATGTTCCAGATGGCACTCTGGTCACTGTGATGGC TGGCAATGATGAAAACTACTCGGCTGAGCTGAGAAATGCTACCGCAGCCATGAAGAAC CAGGTTGCAAGATTTAATGACCTCAGGTTTGTCGGTCGAAGTGGAAGAGGGAAAAGCT TCACTCTGACCATCACTGTCTTCACAAACCCACCGCAAGTCGCCACCTACCACAGAGC CATCAAAATCACAGTGGATGGGCCCCGAGAACCTCGAAGACATCGGCAGAAACTAGAT GATCAGACCAAGCCCGGGAGCTTGTCCTTTTCCGAGCGGCTCAGTGAACTGGAGCAG CTGCGGCGCACAGCCATGAGGGTCAGCCCACACCACCCAGCCCCCACGCCCAACCCT CGTGCCTCCCTGAACCACTCCACTGCCTTTAACCCTCAGCCTCAGAGTCAGATGCAGG ATACAAGGCAGATCCAACCATCCCCACCGTGGTCCTACGATCAGTCCTACCAATACCT GGGATCCATTGCCTCTCCTTCTGTGCACCCAGCAACGCCCATTTCACCTGGACGTGCC AGCGGCATGACAACCCTCTCTGCAGAACTTTCCAGTCGACTCTCAACGGCACCCGACC TGACAGCGTTCAGCGACCCGCGCCAGTTCCCCGCGCTGCCCTCCATCTCCGACCCCC GCATGCACTATCCAGGCGCCTTCACCTACTCCCCGACGCCGGTCACCTCGGGCATCG GCATCGGCATGTCGGCCATGGGCTCGGCCACGCGCTACCACACCTACCTGCCGCCGC CCTACCCCGGCTCGTCGCAAGCGCAGGGAGGCCCGTTCCAAGCCAGCTCGCCCTCCT ACCACCTGTACTACGGCGCCTCGGCCGGCTCCTACCAGTTCTCCATGGTGGGCGGCG AGCGCTCGCCGCCGCGCATCCTGCCGCCCTGCACCAACGCCTCCACCGGCTCCGCG CTGCTCAACCCCAGCCTCCCGAACCAGAGCGACGTGGTGGAGGCCGAGGGCAGCCA CAGCAACTCCCCCACCAACATGGCGCCCTCCGCGCGCCTGGAGGAGGCCGTGTGGA GGCCCTAC (SEQ ID NO:198; NM_001754), or a nucleic acid sequence that hybridizes to a nucleic acid sequence consisting of SEQ ID NO:198 under stringent hybridization conditions.

In some embodiments, runt-related transcription factor 2 (RUNX2) comprises the amino acid sequence: MLHSPHKQPQNHKCGANFLQEDSKKSLVFKWLISAGHYQPPRPTESFKAASSIYNRGYKF YLKKKGGTMASNSLFSTVTPCQQNFFWDPSTSRRFSPPSSSLQPGKMSDVSPVVAAQQQ QQQQQQQQQQQQQQQQQQQQEAAAAAAAAAAAAAAAAAVPRLRPPHDNRTMVEIIADH PAELVRTDSPNFLCSVLPSHWRCNKTLPVAFKVVALGEVPDGTVVTVMAGNDENYSAELR NASAVMKNQVARFNDLRFVGRSGRGKSFTLTITVFTNPPQVATYHRAIKVTVDGPREPRRH RQKLDDSKPSLFSDRLSDLGRIPHPSMRVGVPPQNPRPSLNSAPSPFNPQGQSQITDPRQ AQSSPPWSYDQSYPSYLSQMTSPSIHSTTPLSSTRGTGLPAITDVPRRISDDDTATSDFCL WPSTLSKKSQAGASELGPFSDPRQFPSISSLTESRFSNPRMHYPATFTYTPPVTSGMSLG MSATTHYHTYLPPPYPGSSQSQSGPFQTSSTPYLYYGTSSGSYQFPMVPGGDRSPSRML PPCTTTSNGSTLLNPNLPNQNDGVDADGSHSSSPTVLNSSGRMDESVWRPY (SEQ ID NO:199; NP_001019801), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:199.

In some embodiments, the nucleic acid sequence encoding RUNX2 comprises the nucleic acid sequence ATGCTTCATTCGCCTCACAAACAACCACAGAACCACAAGTGCGGTGCAAACTTTCTCCA GGAGGACAGCAAGAAGTCTCTGGTTTTTAAATGGTTAATCTCCGCAGGTCACTACCAGC CACCGAGACCAACAGAGTCATTTAAGGCTGCAAGCAGTATTTACAACAGAGGGTACAA GTTCTATCTGAAAAAAAAAGGAGGGACTATGGCATCAAACAGCCTCTTCAGCACAGTGA CACCATGTCAGCAAAACTTCTTTTGGGATCCGAGCACCAGCCGGCGCTTCAGCCCCCC CTCCAGCAGCCTGCAGCCCGGCAAAATGAGCGACGTGAGCCCGGTGGTGGCTGCGCA ACAGCAGCAGCAACAGCAGCAGCAGCAACAGCAGCAGCAGCAGCAGCAACAGCAGCA GCAGCAGCAGGAGGCGGCGGCGGCGGCTGCGGCGGCGGCGGCGGCTGCGGCGGC GGCAGCTGCAGTGCCCCGGTTGCGGCCGCCCCACGACAACCGCACCATGGTGGAGAT CATCGCCGACCACCCGGCCGAACTCGTCCGCACCGACAGCCCCAACTTCCTGTGCTC GGTGCTGCCCTCGCACTGGCGCTGCAACAAGACCCTGCCCGTGGCCTTCAAGGTGGT AGCCCTCGGAGAGGTACCAGATGGGACTGTGGTTACTGTCATGGCGGGTAACGATGA AAATTATTCTGCTGAGCTCCGGAATGCCTCTGCTGTTATGAAAAACCAAGTAGCAAGGT TCAACGATCTGAGATTTGTGGGCCGGAGTGGACGAGGCAAGAGTTTCACCTTGACCAT AACCGTCTTCACAAATCCTCCCCAAGTAGCTACCTATCACAGAGCAATTAAAGTTACAG TAGATGGACCTCGGGAACCCAGAAGGCACAGACAGAAGCTTGATGACTCTAAACCTAG TTTGTTCTCTGACCGCCTCAGTGATTTAGGGCGCATTCCTCATCCCAGTATGAGAGTAG GTGTCCCGCCTCAGAACCCACGGCCCTCCCTGAACTCTGCACCAAGTCCTTTTAATCC ACAAGGACAGAGTCAGATTACAGACCCCAGGCAGGCACAGTCTTCCCCGCCGTGGTC CTATGACCAGTCTTACCCCTCCTACCTGAGCCAGATGACGTCCCCGTCCATCCACTCTA CCACCCCGCTGTCTTCCACACGGGGCACTGGGCTTCCTGCCATCACCGATGTGCCTAG GCGCATTTCAGATGATGACACTGCCACCTCTGACTTCTGCCTCTGGCCTTCCACTCTCA GTAAGAAGAGCCAGGCAGGTGCTTCAGAACTGGGCCCTTTTTCAGACCCCAGGCAGTT CCCAAGCATTTCATCCCTCACTGAGAGCCGCTTCTCCAACCCACGAATGCACTATCCAG CCACCTTTACTTACACCCCGCCAGTCACCTCAGGCATGTCCCTCGGTATGTCCGCCAC CACTCACTACCACACCTACCTGCCACCACCCTACCCCGGCTCTTCCCAAAGCCAGAGT GGACCCTTCCAGACCAGCAGCACTCCATATCTCTACTATGGCACTTCGTCAGGATCCTA TCAGTTTCCCATGGTGCCGGGGGGAGACCGGTCTCCTTCCAGAATGCTTCCGCCATGC ACCACCACCTCGAATGGCAGCACGCTATTAAATCCAAATTTGCCTAACCAGAATGATGG TGTTGACGCTGATGGAAGCCACAGCAGTTCCCCAACTGTTTTGAATTCTAGTGGCAGAA TGGATGAATCTGTTTGGCGACCATAT (SEQ ID NO:200; NM_001024630), or a nucleic acid sequence that hybridizes to a nucleic acid sequence consisting of SEQ ID NO:200 under stringent hybridization conditions.

In some embodiments, runt-related transcription factor 3 (RUNX3) comprises the amino acid sequence: MASNSIFDSFPTYSPTFIRDPSTSRRFTPPSPAFPCGGGGGKMGENSGALSAQAAVGPGG RARPEVRSMVDVLADHAGELVRTDSPNFLCSVLPSHWRCNKTLPVAFKVVALGDVPDGTV VTVMAGNDENYSAELRNASAVMKNQVARFNDLRFVGRSGRGKSFTLTITVFTNPTQVATY HRAIKVTVDGPREPRRHRQKLEDQTKPFPDRFGDLERLRMRVTPSTPSPRGSLSTTSHFS SQPQTPIQGTSELNPFSDPRQFDRSFPTLPTLTESRFPDPRMHYPGAMSAAFPYSATPSGT SISSLSVAGMPATSRFHHTYLPPPYPGAPQNQSGPFQANPSPYHLYYGTSSGSYQFSMVA GSSSGGDRSPTRMLASCTSSAASVAAGNLMNPSLGGQSDGVEADGSHSNSPTALSTPGR MDEAVWRPY (SEQ ID NO:201; NP_001026850), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:201.

In some embodiments, the nucleic acid sequence encoding RUNX3 comprises the nucleic acid sequence: ATGGCATCGAACAGCATCTTCGACTCCTTCCCGACCTACTCGCCGACCTTCATCCGCG ACCCAAGCACCAGCCGCCGCTTCACACCTCCCTCCCCGGCCTTCCCCTGCGGCGGCG GCGGCGGCAAGATGGGCGAGAACAGCGGCGCGCTGAGCGCGCAGGCGGCCGTGGG GCCCGGAGGGCGCGCCCGGCCCGAGGTGCGCTCGATGGTGGACGTGCTGGCGGAC CACGCAGGCGAGCTCGTGCGCACCGACAGCCCCAACTTCCTCTGCTCCGTGCTGCCC TCGCACTGGCGCTGCAACAAGACGCTGCCCGTCGCCTTCAAGGTGGTGGCATTGGGG GACGTGCCGGATGGTACGGTGGTGACTGTGATGGCAGGCAATGACGAGAACTACTCC GCTGAGCTGCGCAATGCCTCGGCCGTCATGAAGAACCAGGTGGCCAGGTTCAACGAC CTTCGCTTCGTGGGCCGCAGTGGGCGAGGGAAGAGTTTCACCCTGACCATCACTGTGT TCACCAACCCCACCCAAGTGGCGACCTACCACCGAGCCATCAAGGTGACCGTGGACG GACCCCGGGAGCCCAGACGGCACCGGCAGAAGCTGGAGGACCAGACCAAGCCGTTC CCTGACCGCTTTGGGGACCTGGAACGGCTGCGCATGCGGGTGACACCGAGCACACCC AGCCCCCGAGGCTCACTCAGCACCACAAGCCACTTCAGCAGCCAGCCCCAGACCCCA ATCCAAGGCACCTCGGAACTGAACCCATTCTCCGACCCCCGCCAGTTTGACCGCTCCT TCCCCACGCTGCCAACCCTCACGGAGAGCCGCTTCCCAGACCCCAGGATGCATTATCC CGGGGCCATGTCAGCTGCCTTCCCCTACAGCGCCACGCCCTCGGGCACGAGCATCAG CAGCCTCAGCGTGGCGGGCATGCCGGCCACCAGCCGCTTCCACCATACCTACCTCCC GCCACCCTACCCGGGGGCCCCGCAGAACCAGAGCGGGCCCTTCCAGGCCAACCCGT CCCCCTACCACCTCTACTACGGGACATCCTCTGGCTCCTACCAGTTCTCCATGGTGGC CGGCAGCAGCAGTGGGGGCGACCGCTCACCTACCCGCATGCTGGCCTCTTGCACCAG CAGCGCTGCCTCTGTCGCCGCCGGCAACCTCATGAACCCCAGCCTGGGCGGCCAGAG TGATGGCGTGGAGGCCGACGGCAGCCACAGCAACTCACCCACGGCCCTGAGCACGC CAGGCCGCATGGATGAGGCCGTGTGGCGGCCCTAC (SEQ ID NO:202; NM_001031680), or a nucleic acid sequence that hybridizes to a nucleic acid sequence consisting of SEQ ID NO:202 under stringent hybridization conditions.

In some embodiments, Paired box 9 (PAX9) comprises the amino acid sequence:

(SEQ ID NO: 203; NP_001359005.1)
MEPAFGEVNQLGGVFVNGRPLPNAIRLRIVELAQLGIRPCDISRQLRVS
HGCVSKILARYNETGSILPGAIGGSKPRVTTPTVVKHIRTYKQRDPGIF
AWEIRDRLLADGVCDKYNVPSVSSISRILRNKIGNLAQQGHYDSYKQHQ
PTPQPALPYNHIYSYPSPITAAAAKVPTPPGVPAIPGSVAMPRTWPSSH
SVTDILGIRSITDQVSDSSPYHSPKVEEWSSLGRNNFPAAAPHAVNGLE
KGALEQEAKYGQAPNGLPAVGSFVSASSMAPYPTPAQVSPYMTYSAAPS
GYVAGHGWQHAGGTSLSPHNCDIPASLAFKGMQAAREGSHSVTASAL.,

or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:203.

In some embodiments, the nucleic acid sequence encoding PAX9 comprises the nucleic acid sequence: AGCCCAGCCCACGTTGCTGCTTAGATTGAAATGCAGAACTCAAGCCTCTTTCATCGGG GCACAGACTTCCTTTTACTTCTTCCTTTTGCCCTCTCGCCTCCTCCTCCTGGGAAGAAG CGGAGGCGCCGGCGGTCGGCCGGGATAGCAACAGGCCGGGCCACTGAGGCGGTGC GGAAAGTTTCTGTCTGGGAGTGCGGAACTGGGGCCGGGTTGGTGTACTGCTCGGAGC AATGGAGCCAGCCTTCGGGGAGGTGAACCAGCTGGGAGGAGTGTTCGTGAACGGGAG GCCGCTGCCCAACGCCATCCGGCTTCGCATCGTGGAACTGGCCCAACTGGGCATCCG ACCGTGTGACATCAGCCGCCAGCTACGGGTCTCGCACGGCTGCGTCAGCAAGATCCT GGCGCGATACAACGAGACGGGCTCGATCTTGCCAGGAGCCATCGGGGGCAGCAAGC CCCGGGTCACTACCCCCACCGTGGTGAAACACATCCGGACCTACAAGCAGAGAGACC CCGGCATCTTCGCCTGGGAGATCCGGGACCGCCTGCTGGCGGACGGCGTGTGCGAC AAGTACAATGTGCCCTCCGTGAGCTCCATCAGCCGCATTCTGCGCAACAAGATCGGCA ACTTGGCCCAGCAGGGTCATTACGACTCATACAAGCAGCACCAGCCGACGCCGCAGC CAGCGCTGCCCTACAACCACATCTACTCGTACCCCAGCCCTATCACGGCGGCGGCCG CCAAGGTGCCCACGCCACCCGGGGTGCCTGCCATCCCCGGTTCGGTGGCCATGCCG CGCACCTGGCCCTCCTCGCACTCCGTCACCGACATCCTGGGCATCCGCTCCATCACC GACCAAGTGAGCGACAGCTCCCCCTACCACAGCCCCAAGGTGGAGGAGTGGAGCAGC CTGGGCCGCAACAACTTCCCCGCCGCCGCCCCGCACGCGGTGAACGGGTTGGAGAA GGGAGCCCTGGAGCAGGAAGCCAAGTACGGTCAGGCACCAAATGGTCTCCCAGCTGT GGGCAGTTTTGTGTCAGCATCCAGCATGGCTCCTTACCCTACCCCAGCCCAAGTGTCG CCTTACATGACCTACAGTGCTGCTCCTTCTGGTTATGTTGCTGGACATGGGTGGCAACA TGCTGGGGGCACCTCATTGTCTCCCCACAACTGTGACATTCCGGCATCGCTGGCGTTC AAGGGAATGCAGGCAGCCAGAGAAGGTAGTCATTCTGTCACGGCTTCCGCGCTCTGAT GGGAAATTCCGTCTCCAGCAGCTTCACCCGGGTCTCCCTGTCTCAGCACCTCCTCCCC CAATTCCCAGGTCTCACATCCCACCCCTCCTGCCCTCCAACCCTTCTGCCTTGAAAGCT GGCTGTACGGACTCACATCCTTTGTGCTAATGACACTTACATATTTCTTGCCATAACTTT TCTCTTGCAGAAAAACTGACATGACTTTAGGATTTAAAAACAAGAGCAACAATAAGCATT GAATGAGACATTTGTGTTGCCCACATACTGTCTTAACATAACAAAGAAACCTACACCCC TCAAAGGGTTTAAGGAACTTTACAAACTAGTCTTTGGTAAAACCACATGTGTATATTTAT TCTAAATCAACCTGAACTTTTGAAATGTGCAATTGTTGAGATTTTGCAAAATCAATAAAG GAAAATACTTATAGAAAAAATTATGCTACACCCTCTAATCAAATATGGTAACCAAGTAAG CTTTAATTCATCATTAGGAAACAAATCAATAAGTGACTTGTTTGAGTGATCCTTTGTTTAA GACATGACCTATTTTGTTGAAAAATATATGTAGAACCCAAGCAATATCTGAATCTAGCTC TCCCTGGTGTTTTGACTTGGTTCCAAATACAATAATGTTTATATTTTCTATTAGTTTGTAA ATACGGACTCTGGATGGTGCATTTGTGTCTTCATTCCATAAGATATTCCCCTCCCCTCA GCCCCACCCCCTCTCTATTTTTTTCTTTCTTTTTTGCAAAGGTGACTTTCTGGCAACGTC TTTGTCTCTGTTTGGTGGTGGGCTGCTCGGGCTCCTGGACCTGGACTTGCCCCCAAAT TTTGTGTATGCAGTGAAGGCTTCAACATCTCATGAAGGACACTTTATTTCTACAGCAGA GGACACGAAAAACAGATAAAACAAGCCAGTCTCCCATTTTGTACCTAATCAAACAACAC ACATGCTAAGCATATAAAGACAAGAGGGTGGAAAATATCTGAACAAGAAGGCTCTAAAG GAAGTCACTTAGAAACTTAAGTTTAATGTGAAATGTTTTGCAAAGATGCTTAAAATGAAC TTTGTGTTAAGAAAACCACTGTGAAACTAAATTGTCCTATTATTGTTGGCTTACCTGTGT GTTCAGCAATCTCAGCCCCAAATAATGTTGTAATTTAAAGAAAATGGAAAATTCTGCTCT AATGAATGTAACAATGGCTTGCTGTGAAGTTTACATTGTTGTACAGAAGCATGTTTCGCA TGTAGGTAAACTGGTGGTGGTACTAGAAATACAATGTTATTTAATTTTAACAAATTCCCT TTATTCATTTCTGAAATTACAGGACACAGTTTAACTCATAAACCTTTCTAGACCAATTTAT TTTTCACTTTAATGTTAATAACAGTTGTGGAGTATATGTGTGTGTGAGCATGTGAGTATG TGTTGTATTTTAAAACAATTGATTTTCTGGGGCAAAATTCTACAGTTTTTAATCCCTTCTG TTTAGGAAGTTCTTCCTGTTTGGCAATATAGGCTTAAAAATATGTTTTTAGGACATTGGT ACAATTCAGCTGTTGGAAAATTAATATATTGAGGGTTTTTTGGTACTAATTCTGTGCAAT AACTAAAAGAGCACCTCACTGGATATGGATGTTGAAGATGGATTCCCTAGGTGATTTTA ATTTCTTCCGGTCTGTGCTGTGCACAGTCTACATGGCAATGCGGTTCCACCACATCGGT TTCGTGGCTTCGTTTAAAACTCAGATGGCTAGATTAGTTAGGTTTTCAAATCACTAGGAT GTAAACAGTAAGCAGATTTCTGACACACAAATTATGTTAGAGTGACTGCTTTTTTCAGAC AGCAGATATCTTATAGAGAGCTTTGAACTGCATTTATTTCTAAAGCAACCGAAATTCAGT GCTACAAATAGAGGATTATAACTTCAGGAGAAGAATAAGCAGAAGGAGCAGATGAACTC TCAGGGCCATAGTCTTCCTTTGATCTTGTAAAACTTCCATTGACATCTGGAGTTCCCAGT CTGGTGAGAAAATAGACTATAAACTGAATGGAACAAAGATCCAATCCAATATTTTGGTG GAGACTTCTTTAAAACCATACCATACAGGGACTCTCCTGTCATCTGAAAAACTGATGTAA GGTACAGAACTATTCTTTATCAAATGTTTTTAGGTGGCTGTTAGGGGGCTTTAAAAAATA TTACTTGCTTGTGTGGAAATGCAAATAATGTTATTTTCTTTATCTAAATTAAGAAATCTCT TGTTATTGTGCTATTTATAATTTTTTTCTGGTTCTTGTATTTTAAAAAATCTAATATTAATG GTATTGAAGTTTCCTTTTCTCCCTCTAGGTCTTAACAGTGAATTCACATGGAGTAATTTT TAAAAGATATCAGATACAATTTGCTATTCAAAGAAAATTATGATTTAAAGCCACTTTTTAA AATACGAGAAGGAAAATAGGATGGATTAAAGGGTTAACTTTTAAAGATTATTATTGGTTA ATGTTGACATATTTCCTCTATCTCATAGATGGTAAAAGTGTTGCTTTTAAACTGGCAAAT GCACTCTTCAGAAATCCTTTTCTATCTGATCCACATGGAGAGGTTAAAGGTTCAATTTCA TGACCTCTATGCAGGCAGCGCTCTCATTGGATGTAAGAATATTACCTGCAAGGATAGAA TGCAGTTGTGCAACAGAGACACATTCTTATTTCTTTTTTTTCACAATTTTGTTTTGTTTTT AATGACCCTTTTATTGAATATTGGACTGAAATATAAATTTTAAAAAACACGTTGGAAAGG ATGTACAACAGAAGGCTATGTATGTATATACAGTATGTCAAAAGCCTTTTATTTTTATACT TCAAATGCTCTAAATTAATAAAAAGTAATAATTA. (SEQ ID NO:204; NM_001372076.1), or a nucleic acid sequence that hybridizes to a nucleic acid sequence consisting of SEQ ID NO:204 under stringent hybridization conditions.

In some embodiments, Homeobox Protein Nkx-3.2 (Nkx-3.2) comprises the amino acid sequence: MAVRGANTLTSFSIQAILNKKEERGGLAAPEGRPAPGGTAASVAAAPAVCCWRLFGERDA GALGGAEDSLLASPAGTRTAAGRTAESPEGWDSDSALSEENESRRRCADARGASGAGLA GGSLSLGQPVCELAASKDLEEEAAGRSDSEMSASVSGDRSPRTEDDGVGPRGAHVSALC SGAGGGGGSGPAGVAEEEEEPAAPKPRKKRSRAAFSHAQVFELERRFNHQRYLSGPERA DLAASLKLTETQVKIWFQNRRYKTKRRQMAADLLASAPAAKKVAVKVLVRDDQRQYLPGE VLRPPSLLPLQPSYYYPYYCLPGWALSTCAAAAGTQ. (SEQ ID NO:205; NP_001180.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:205

In some embodiments, the nucleic acid sequence encoding Nkx-3.2 comprises the nucleic acid sequence: ACTCGCGCTGCGGCCGCCGGCGCTCTCTGTCCCGCTCGGAGCTGCTCGGCGCCCCA GCTGCCCGCCCCGCCGGCCGCTCCTGCCCGCGGCGCAGATGGCTGTGCGCGGCGCC AACACCTTGACGTCCTTCTCCATCCAGGCGATCCTCAACAAGAAAGAGGAGCGCGGCG GGCTGGCCGCGCCAGAGGGGCGCCCGGCGCCCGGGGGCACAGCGGCATCGGTGGC CGCGGCTCCCGCTGTCTGCTGTTGGCGGCTCTTTGGGGAGAGGGACGCGGGCGCGT TGGGGGGCGCCGAGGACTCTCTGCTGGCGTCTCCTGCCGGTACCAGAACAGCTGCGG GGCGGACTGCGGAGAGCCCGGAAGGCTGGGACTCGGACTCCGCGCTCAGCGAGGAG AACGAGAGCAGGCGGCGCTGCGCGGACGCGCGGGGGGCCAGCGGGGCCGGCCTTG CGGGGGGATCCTTGAGCCTCGGCCAGCCGGTCTGTGAGCTGGCCGCTTCCAAAGACC TAGAGGAGGAAGCCGCGGGCCGGAGCGACAGCGAGATGTCCGCCAGCGTCTCAGGC GACCGCAGCCCAAGGACCGAGGACGACGGTGTTGGCCCCAGAGGTGCACACGTGTC CGCGCTGTGCAGCGGGGCCGGCGGCGGGGGCGGCAGCGGGCCGGCAGGCGTCGC GGAGGAGGAGGAGGAGCCGGCGGCGCCCAAGCCACGCAAGAAGCGCTCGCGGGCC GCTTTCTCCCACGCGCAGGTCTTCGAGCTGGAGCGCCGCTTTAACCACCAGCGCTACC TGTCCGGGCCCGAGCGCGCAGACCTGGCCGCGTCGCTGAAGCTCACCGAGACGCAG GTGAAAATCTGGTTCCAGAACCGTCGCTACAAGACAAAGCGCCGGCAGATGGCAGCC GACCTGCTGGCCTCGGCGCCCGCCGCCAAGAAGGTGGCCGTAAAGGTGCTGGTGCG CGACGACCAGAGACAATACCTGCCCGGCGAAGTGCTGCGGCCACCCTCGCTTCTGCC ACTGCAGCCCTCCTACTATTACCCGTACTACTGCCTCCCAGGCTGGGCGCTCTCCACC TGCGCAGCTGCCGCAGGCACCCAGTGAACCCGCTTGGGCTGAGGCAGCGAGTGATTC CCGCGCTCCGGCTCCGGACCGGCGCTGACAGCTGTAGGCTGTAGCCTGCACGGGGC GCCCCGCCAAGGAGGCACCTGGAGGTGAAACCCAGCTCCAGCTCCCGTTAGCCAGGA CTTGTCCCCTGGCAGCTGGGCTGAGTCTGCCCTGAGGGGGCGCCTTTTTCTAATTTGA ACAGAGGCACCCTATGGCCTAGGGGCCCTGATCGCCCACCTGCCTGGAAGCCCCTGG GCTCTATTTATTATCATGACAATGTTGGAATTAAATTTTGATTCGAATATGTCTGCCTGG GGGTGGGGTTTTCCCTGAGCGGCAACTCCTGGAGACCACATAGCCTGAATCCTCAGAA TTTCAGGCCTGCTGGGAGCTTTCTGCACTAGGCCACACTAGTTCATGGTATCCATGCTA CCAATCTATGTGTATCTACATATCTTTTATTTTTGGAAATTGCATTTGTAACCAAGGGGT GCGAAACCCTGGCAGTCCCAGGCAGCACCAGGCCAGGGGTTGATTTGAAACGTGAAG GATTGGGTTTTCAGGCCCTCTGCTCCACCCCTCCTGTGTGTCAGAGCTAGGGTGGGGG TGCCCGATTCGGGTGCTGAATGTAAGGAGGGGAGCCTCCAAGTGTGGTGCAAGCCGG GGGTCTCCACATCTTCCTTCTCTGAAGTCCAGGTACCTGCACAAGCAGGAAGCGCCTG GGAGTCCCGGAAGGAGGAGAGCGCACACCCAGGCAGCCCTCTGCGGAAACTTTCCTT GGTTTCTTTTTATTTGTGTAAAGGAGGTTAAGACGTGTCGCACTTTTCAGTTGTTTGTAT TCAAATGACGATTATTTTTCTACTCAATGTGAATATCCCTGGCCAGCCTTTCCACGGCG CCCACCGCAGTGCCGCTGCCTGGCCCTCAGTGTCTACCTTCTGCCCTCTGCGACTCCA GTGCTCTGGCCCGGGACTCCCCTATCCGCCCCTCACTTACCCTTAAACAGGTGATCCC ACCTGTCTTGTCAACCTCGCCGCTTTTCGCCTCCTTAATGGCACTGTGCACTCAACTAG AGTATTAACTGTAAAAAGATTTGTGAAGTTTGGAAGCTCTATTCGCTGTATTTTTTCTTTA ATTTATAAACTTTTAGTTTAACATGC. (SEQ ID NO:206; NM_001189.4), or a nucleic acid sequence that hybridizes to a nucleic acid sequence consisting of SEQ ID NO:206 under stringent hybridization conditions.

In some embodiments, AP-1 transcription factor (FOS, C-FOX, AP-1, p55) comprises the amino acid sequence: MMFSGFNADYEASSSRCSSASPAGDSLSYYHSPADSFSSMGSPVNAQDFCTDLAVSSAN FIPTVTAISTSPDLQWLVQPALVSSVAPSQTRAPHPFGVPAPSAGAYSRAGVVKTMTGGRA QSIGRRGKVEQLSPEEEEKRRIRRERNKMAAAKCRNRRRELTDTLQAETDQLEDEKSALQ TEIANLLKEKEKLEFILAAHRPACKIPDDLGFPEEMSVASLDLTGGLPEVATPESEEAFTLPLL NDPEPKPSVEPVKSISSMELKTEPFDDFLFPASSRPSGSETARSVPDMDLSGSFYAADWE PLHSGSLGMGPMATELEPLCTPVVTCTPSCTAYTSSFVFTYPEADS FPSCAAAHRKGSSSNEPSSDSLSSPTLLAL. (SEQ ID NO:207; NP_005243.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:207) In some embodiments, the nucleic acid sequence encoding FOS comprises the nucleic acid sequence: AACCGCATCTGCAGCGAGCATCTGAGAAGCCAAGACTGAGCCGGCGGCCGCGGCGCA GCGAACGAGCAGTGACCGTGCTCCTACCCAGCTCTGCTCCACAGCGCCCACCTGTCT CCGCCCCTCGGCCCCTCGCCCGGCTTTGCCTAACCGCCACGATGATGTTCTCGGGCT TCAACGCAGACTACGAGGCGTCATCCTCCCGCTGCAGCAGCGCGTCCCCGGCCGGGG ATAGCCTCTCTTACTACCACTCACCCGCAGACTCCTTCTCCAGCATGGGCTCGCCTGTC AACGCGCAGGACTTCTGCACGGACCTGGCCGTCTCCAGTGCCAACTTCATTCCCACGG TCACTGCCATCTCGACCAGTCCGGACCTGCAGTGGCTGGTGCAGCCCGCCCTCGTCT CCTCCGTGGCCCCATCGCAGACCAGAGCCCCTCACCCTTTCGGAGTCCCCGCCCCCT CCGCTGGGGCTTACTCCAGGGCTGGCGTTGTGAAGACCATGACAGGAGGCCGAGCGC AGAGCATTGGCAGGAGGGGCAAGGTGGAACAGTTATCTCCAGAAGAAGAAGAGAAAA GGAGAATCCGAAGGGAAAGGAATAAGATGGCTGCAGCCAAATGCCGCAACCGGAGGA GGGAGCTGACTGATACACTCCAAGCGGAGACAGACCAACTAGAAGATGAGAAGTCTGC TTTGCAGACCGAGATTGCCAACCTGCTGAAGGAGAAGGAAAAACTAGAGTTCATCCTG GCAGCTCACCGACCTGCCTGCAAGATCCCTGATGACCTGGGCTTCCCAGAAGAGATGT CTGTGGCTTCCCTTGATCTGACTGGGGGCCTGCCAGAGGTTGCCACCCCGGAGTCTG AGGAGGCCTTCACCCTGCCTCTCCTCAATGACCCTGAGCCCAAGCCCTCAGTGGAACC TGTCAAGAGCATCAGCAGCATGGAGCTGAAGACCGAGCCCTTTGATGACTTCCTGTTC CCAGCATCATCCAGGCCCAGTGGCTCTGAGACAGCCCGCTCCGTGCCAGACATGGAC CTATCTGGGTCCTTCTATGCAGCAGACTGGGAGCCTCTGCACAGTGGCTCCCTGGGGA TGGGGCCCATGGCCACAGAGCTGGAGCCCCTGTGCACTCCGGTGGTCACCTGTACTC CCAGCTGCACTGCTTACACGTCTTCCTTCGTCTTCACCTACCCCGAGGCTGACTCCTTC CCCAGCTGTGCAGCTGCCCACCGCAAGGGCAGCAGCAGCAATGAGCCTTCCTCTGAC TCGCTCAGCTCACCCACGCTGCTGGCCCTGTGAGGGGGCAGGGAAGGGGAGGCAGC CGGCACCCACAAGTGCCACTGCCCGAGCTGGTGCATTACAGAGAGGAGAAACACATCT TCCCTAGAGGGTTCCTGTAGACCTAGGGAGGACCTTATCTGTGCGTGAAACACACCAG GCTGTGGGCCTCAAGGACTTGAAAGCATCCATGTGTGGACTCAAGTCCTTACCTCTTC CGGAGATGTAGCAAAACGCATGGAGTGTGTATTGTTCCCAGTGACACTTCAGAGAGCT GGTAGTTAGTAGCATGTTGAGCCAGGCCTGGGTCTGTGTCTCTTTTCTCTTTCTCCTTA GTCTTCTCATAGCATTAACTAATCTATTGGGTTCATTATTGGAATTAACCTGGTGCTGGA TATTTTCAAATTGTATCTAGTGCAGCTGATTTTAACAATAACTACTGTGTTCCTGGCAATA GTGTGTTCTGATTAGAAATGACCAATATTATACTAAGAAAAGATACGACTTTATTTTCTG GTAGATAGAAATAAATAGCTATATCCATGTACTGTAGTTTTTCTTCAACATCAATGTTCAT TGTAATGTTACTGATCATGCATTGTTGAGGTGGTCTGAATGTTCTGACATTAACAGTTTT CCATGAAAACGTTTTATTGTGTTTTTAATTTATTTATTAAGATGGATTCTCAGATATTTATA TTTTTATTTTATTTTTTTCTACCTTGAGGTCTTTTGACATGTGGAAAGTGAATTTGAATGA AAAATTTAAGCATTGTTTGCTTATTGTTCCAAGACATTGTCAATAAAAGCATTTAAGTTGA ATGCGA. (SEQ ID NO:208; NM_005252.4), or a nucleic acid sequence that hybridizes to a nucleic acid sequence consisting of SEQ ID NO:208 under stringent hybridization conditions.

In some embodiments, FosB proto-oncogene (FosB, GOS3, GOS3, GOSB) comprises the amino acid sequence: MFQAFPGDYDSGSRCSSSPSAESQYLSSVDSFGSPPTAAASQECAGLGEMPGSFVPTVT AITTSQDLQWLVQPTLISSMAQSQGQPLASQPPVVDPYDMPGTSYSTPGMSGYSSGGAS GSGGPSTSGTTSGPGPARPARARPRRPREETETDQLEEEKAELESEIAELQKEKERLEFVL VAH KPGCKIPYEEGPGPGPLAEVRDLPGSAPAKEDGFSWLLPPPPPPPLPFQTSQDAPPN LTASLFTHSEVQVLGDPFPVVNPSYTSSFVLTCPEVSAFAGAQRTSGSDQPSDPLNSPSLL AL. (SEQ ID NO:209; NP_001107643.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:209) In some embodiments, the nucleic acid sequence encoding FosB comprises the nucleic acid sequence: ATTCATAAGACTCAGAGCTACGGCCACGGCAGGGACACGCGGAACCAAGACTTGGAAA CTTGATTGTTGTGGTTCTTCTTGGGGGTTATGAAATTTCATTAATCTTTTTTTTTCCGGG GAGAAAGTTTTTGGAAAGATTCTTCCAGATATTTCTTCATTTTCTTTTGGAGGACCGACT TACTTTTTTTGGTCTTCTTTATTACTCCCCTCCCCCCGTGGGACCCGCCGGACGCGTGG AGGAGACCGTAGCTGAAGCTGATTCTGTACAGCGGGACAGCGCTTTCTGCCCCTGGG GGAGCAACCCCTCCCTCGCCCCTGGGTCCTACGGAGCCTGCACTTTCAAGAGGTACA GCGGCATCCTGTGGGGGCCTGGGCACCGCAGGAAGACTGCACAGAAACTTTGCCATT GTTGGAACGGGACGTTGCTCCTTCCCCGAGCTTCCCCGGACAGCGTACTTTGAGGACT CGCTCAGCTCACCGGGGACTCCCACGGCTCACCCCGGACTTGCACCTTACTTCCCCAA CCCGGCCATAGCCTTGGCTTCCCGGCGACCTCAGCGTGGTCACAGGGGCCCCCCTGT GCCCAGGGAAATGTTTCAGGCTTTCCCCGGAGACTACGACTCCGGCTCCCGGTGCAG CTCCTCACCCTCTGCCGAGTCTCAATATCTGTCTTCGGTGGACTCCTTCGGCAGTCCAC CCACCGCCGCCGCCTCCCAGGAGTGCGCCGGTCTCGGGGAAATGCCCGGTTCCTTCG TGCCCACGGTCACCGCGATCACAACCAGCCAGGACCTCCAGTGGCTTGTGCAACCCA CCCTCATCTCTTCCATGGCCCAGTCCCAGGGGCAGCCACTGGCCTCCCAGCCCCCGG TCGTCGACCCCTACGACATGCCGGGAACCAGCTACTCCACACCAGGCATGAGTGGCTA CAGCAGTGGCGGAGCGAGTGGCAGTGGTGGGCCTTCCACCAGCGGAACTACCAGTG GGCCTGGGCCTGCCCGCCCAGCCCGAGCCCGGCCTAGGAGACCCCGAGAGGAGACG GAGACAGATCAGTTGGAGGAAGAAAAAGCAGAGCTGGAGTCGGAGATCGCCGAGCTC CAAAAGGAGAAGGAACGTCTGGAGTTTGTGCTGGTGGCCCACAAACCGGGCTGCAAG ATCCCCTACGAAGAGGGGCCCGGGCCGGGCCCGCTGGCGGAGGTGAGAGATTTGCC GGGCTCAGCACCGGCTAAGGAAGATGGCTTCAGCTGGCTGCTGCCGCCCCCGCCACC ACCGCCCCTGCCCTTCCAGACCAGCCAAGACGCACCCCCCAACCTGACGGCTTCTCTC TTTACACACAGTGAAGTTCAAGTCCTCGGCGACCCCTTCCCCGTTGTTAACCCTTCGTA CACTTCTTCGTTTGTCCTCACCTGCCCGGAGGTCTCCGCGTTCGCCGGCGCCCAACGC ACCAGCGGCAGTGACCAGCCTTCCGATCCCCTGAACTCGCCCTCCCTCCTCGCTCTGT GAACTCTTTAGACACACAAAACAAACAAACACATGGGGGAGAGAGACTTGGAAGAGGA GGAGGAGGAGGAGAAGGAGGAGAGAGAGGGGAAGAGACAAAGTGGGTGTGTGGCCT CCCTGGCTCCTCCGTCTGACCCTCTGCGGCCACTGCGCCACTGCCATCGGACAGGAG GATTCCTTGTGTTTTGTCCTGCCTCTTGTTTCTGTGCCCCGGCGAGGCCGGAGAGCTG GTGACTTTGGGGACAGGGGGTGGGAAGGGGATGGACACCCCCAGCTGACTGTTGGCT CTCTGACGTCAACCCAAGCTCTGGGGATGGGTGGGGAGGGGGGCGGGTGACGCCCA CCTTCGGGCAGTCCTGTGTGAGGATTAAGGGACGGGGGTGGGAGGTAGGCTGTGGG GTGGGCTGGAGTCCTCTCCAGAGAGGCTCAACAAGGAAAAATGCCACTCCCTACCCAA TGTCTCCCACACCCACCCTTTTTTTGGGGTGCCTAGGTTGGTTTCCCCTGCACTCCCGA CCTTAGCTTATTGATCCCACATTTCCATGGTGTGAGATCCTCTTTACTCTGGGCAGAAG TGAGCCCCCCCCTTAAAGGGAATTCGATGCCCCCCTAGAATAATCTCATCCCCCCACC CGACTTCTTTTGAAATGTGAACGTCCTTCCTTGACTGTCTAGCCACTCCCTCCCAGAAA AACTGGCTCTGATTGGAATTTCTGGCCTCCTAAGGCTCCCCACCCCGAAATCAGCCCC CAGCCTTGTTTCTGATGACAGTGTTATCCCAAGACCCTGCCCCCTGCCAGCCGACCCT CCTGGCCTTCCTCGTTGGGCCGCTCTGATTTCAGGCAGCAGGGGCTGCTGTGATGCC GTCCTGCTGGAGTGATTTATACTGTGAAATGAGTTGGCCAGATTGTGGGGTGCAGCTG GGTGGGGCAGCACACCTCTGGGGGGATAATGTCCCCACTCCCGAAAGCCTTTCCTCG GTCTCCCTTCCGTCCATCCCCCTTCTTCCTCCCCTCAACAGTGAGTTAGACTCAAGGGG GTGACAGAACCGAGAAGGGGGTGACAGTCCTCCATCCACGTGGCCTCTCTCTCTCTCC TCAGGACCCTCAGCCCTGGCCTTTTTCTTTAAGGTCCCCCGACCAATCCCCAGCCTAG GACGCCAACTTCTCCCACCCCTTGGCCCCTCACATCCTCTCCAGGAAGGGAGTGAGG GGCTGTGACATTTTTCCGGAGAAGATTTCAGAGCTGAGGCTTTGGTACCCCCAAACCC CCAATATTTTTGGACTGGCAGACTCAAGGGGCTGGAATCTCATGATTCCATGCCCGAGT CCGCCCATCCCTGACCATGGTTTTGGCTCTCCCACCCCGCCGTTCCCTGCGCTTCATC TCATGAGGATTTCTTTATGAGGCAAATTTATATTTTTTAATATCGGGGGGTGGACCACGC CGCCCTCCATCCGTGCTGCATGAAAAACATTCCACGTGCCCCTTGTCGCGCGTCTCCC ATCCTGATCCCAGACCCATTCCTTAGCTATTTATCCCTTTCCTGGTTTCCGAAAGGCAAT TATATCTATTATGTATAAGTAAATATATTATATATGGATGTGTGTGTGTGCGTGCGCGTG AGTGTGTGAGCGCTTCTGCAGCCTCGGCCTAGGTCACGTTGGCCCTCAAAGCGAGCC GTTGAATTGGAAACTGCTTCTAGAAACTCTGGCTCAGCCTGTCTCGGGCTGACCCTTTT CTGATCGTCTCGGCCCCTCTGATTGTTCCCGATGGTCTCTCTCCCTCTGTCTTTTCTCC TCCGCCTGTGTCCATCTGACCGTTTTCACTTGTCTCCTTTCTGACTGTCCCTGCCAATG CTCCAGCTGTCGTCTGACTCTGGGTTCGTTGGGGACATGAGATTTTATTTTTTGTGAGT GAGACTGAGGGATCGTAGATTTTTACAATCTGTATCTTTGACAATTCTGGGTGCGAGTG TGAGAGTGTGAGCAGGGCTTGCTCCTGCCAACCACAATTCAATGAATCCCCGACCCCC CTACCCCATGCTGTACTTGTGGTTCTCTTTTTGTATTTTGCATCTGACCCCGGGGGGCT GGGACAGATTGGCAATGGGCCGTCCCCTCTCCCCTTGGTTCTGCACTGTTGCCAATAA AAAGCTCTTAAAAACGCA (SEQ ID NO:210; NM_001114171.2), or a nucleic acid sequence that hybridizes to a nucleic acid sequence consisting of SEQ ID NO:210 under stringent hybridization conditions.

In some embodiments, FOS like 1, AP-1 transcription factor subunit (FRA, FRA1, FOSL1) comprises the amino acid sequence: MFRDFGEPGPSSGNGGGYGGPAQPPAAAQAAQQKFHLVPSINTMSGSQELQWMVQPHF LGPSSYPRPLTYPQYSPPQPRPGVIRALGPPPGVRRRPCEQETDKLEDEKSGLQREIEELQ KQKERLELVLEAHRPICKIPEGAKEGDTGSTSGTSSPPAPCRPVPCISLSPGPVLEPEALHT PTLMTTPSLTPFTPSLVFTYPSTPEPCASAHRKSSSSSGDPSSDPLGSPTLLAL. (SEQ ID NO:211; NP_001287773.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:211)

In some embodiments, the nucleic acid sequence encoding FRA comprises the nucleic acid sequence: GAGTCAGAACCCAGCAGCCGTGTACCCCGCAGAGCCGCCAGCCCCGGGCATGTTCCG AGACTTCGGGGAACCCGGCCCGAGCTCCGGGAACGGCGGCGGGTACGGCGGCCCCG CGCAGCCCCCGGCCGCAGCGCAGGCAGCCCAGCAGAAGTTCCACCTGGTGCCAAGC ATCAACACCATGAGTGGCAGTCAGGAGCTGCAGTGGATGGTACAGCCTCATTTCCTGG GGCCCAGCAGTTACCCCAGGCCTCTGACCTACCCTCAGTACAGCCCCCCACAACCCC GGCCAGGAGTCATCCGGGCCCTGGGGCCGCCTCCAGGGGTACGTCGAAGGCCTTGT GAACAGGAGACTGACAAACTGGAAGATGAGAAATCTGGGCTGCAGCGAGAGATTGAG GAGCTGCAGAAGCAGAAGGAGCGCCTAGAGCTGGTGCTGGAAGCCCACCGACCCATC TGCAAAATCCCGGAAGGAGCCAAGGAGGGGGACACAGGCAGTACCAGTGGCACCAGC AGCCCACCAGCCCCCTGCCGCCCTGTACCTTGTATCTCCCTTTCCCCAGGGCCTGTGC TTGAACCTGAGGCACTGCACACCCCCACACTCATGACCACACCCTCCCTAACTCCTTTC ACCCCCAGCCTGGTCTTCACCTACCCCAGCACTCCTGAGCCTTGTGCCTCAGCTCATC GCAAGAGTAGCAGCAGCAGCGGAGACCCATCCTCTGACCCCCTTGGCTCTCCAACCCT CCTCGCTTTGTGAGGCGCCTGAGCCCTACTCCCTGCAGATGCCACCCTAGCCAATGTC TCCTCCCCTTCCCCCACCGGTCCAGCTGGCCTGGACAGTATCCCACATCCAACTCCAG CAACTTCTTCTCCATCCCTCTAATGAGACTGACCATATTGTGCTTCACAGTAGAGCCAG CTTGGGGCCACCAAAGCTGCCCACTGTTTCTCTTGAGCTGGCCTCTCTAGCACAATTTG CACTAAATCAGAGACAAAATATTTCCCATTTGTGCCAGAGGAATCCTGGCAGCCCAGAG ACTTTGTAGATCCTTAGAGGTCCTCTGGAGCCCTAACCCCTTCCAGATCACTGCCACAC TCTCCATCACCCTCTTCCTGTGATCCACCCAACCCTATCTCCTGACAGAAGGTGCCACT TTACCCACCTAGAACACTAACTCACCAGCCCCACTGCCAGCAGCAGCAGGTGATTGGA CCAGGCCATTCTGCCGCCCCCTCCTGAACCGCACAGCTCAGGAGGCGCCCTTGGCTT CTGTGATGAGCTGATCTGCGGATCTCAGCTTTGAGAAGCCTTCAGCTCCAGGGAATCC AAGCCTCCACAGCGAGGGCAGCTGCTATTTATTTTCCTAAAGAGAGTATTTTTATACAAA CCTACCAAAATGGAATAAAAGGCTTGAAGCTGTGGCCTGAGTGCCTCACTGGACCCAG AGGCCAATGGGAGAGTATTTGGAGCCCTAGGTCCCAGCCTTAGCTCTACAGACTCACT GCATGACCTTGGACAAATTCTTTGATATTTTTGGACTTTGTCTTATCTGACAAGTGGGGC TACATCCGCTCGGCCTCATCTCCGGGACTG (SEQ ID NO:212; NM_001300844.2), or a nucleic acid sequence that hybridizes to a nucleic acid sequence consisting of SEQ ID NO:212 under stringent hybridization conditions.

In some embodiments, FOS like 2, AP-1 transcription factor subunit (FRA2, FOSL2) comprises the amino acid sequence: MYQDYPGNFDTSSRGSSGSPAHAESYSSGGGGQQKFRVDMPGSGSAFIPTINAITTSQDL QWMVQPTVITSMSNPYPRSHPYSPLPGLASVPGHMALPRPGVIKTIGTTVGRRRRDEQLS PEEEEKRRIRRERNKLAAAKCRNRRRELTEKLQAETEELEEEKSGLQKEIAELQKEKEKLEF MLVAHGPVCKISPEERRSPPAPGLQPMRSGGGSVGAVVVKQEPLEEDSPSSSSAGLDKA QRSVIKPISIAGGFYGEEPLHTPIVVTSTPAVTPGTSNLVFTYPSVLEQESPASPSESCSKAH RRSSSSGDQSSDSLNSPTLLAL. (SEQ ID NO:213; NP_005244.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:213)

In some embodiments, the nucleic acid sequence encoding FRA2 comprises the nucleic acid sequence: GTAGTGACTCATCTCGGGCAGAGCGCTAGGGCTCCGAGCGAACCAGCGAGCGAGCGA ACGAGCGGCGCTCGGCGGGGACAGAAAGAGGGAGAGAGAGAGAGAGAGAGAGGGAG AGGCGCGGCCGGGCGAGGCGGGCCCGTCCGGGAGCGGGCTCCGGGGAAGGGGTGC GGGTCTGGGCGCCGGAGCGGGGAGCGGGGCCGCGTCCCTCTCAGCGCCAGCTCTAC TTGAGCCCCACGAGCCGCTGTCCCCCTGGCGCGCTCGGGGCCGCGGGACGGGCGCA CGCCGCCTTCTCCTAGTCAAGTATCCGAGCCGCCCCGAAACTCGGGCGGCGAGTCGG CCACGGGAAGTTTATTCTCCGGCTCCTTTTCTAAAAGGAAGAAACAGAAGTTTCTCCCA GCGGACAGCTTTTCTTTCCGCCTTTTTGGCCCTGTCTGAAATCGGGGGTCCCCAGGGC TGGCAGGCCAGGCTCGCTGGGCTCCTAATCTTTTTTTTAATTTCCAATTTTTGATTGGG CCGTGGGTCCCCGCTGAGCTCCGGCTGCGCGCGGGGGCGGGAGGGCGCGCGCAGG GGAGGGACCGAGAGACGCGCCGACTTTTTAGAGGGAGGGATCGGGTGGACAACTGGT CCCGCGGCGCTCGCAGAGCCGGAAAGAAGTGCTGTAAGGGACGCTCGGGGGACGCT GTTCCTGAGGTGTCGCCGCCTCCCTGTCCTCGCCCTCCGCGGTGGGGGAGAAACCCA GGAGCGAAGCCCAGAGCCCGCGGCGCGGCCGGCGGACGAACGAGCGCGCAGCAGC CGGTGCGCGGCCGCGGCGAGGGCGGGGGAAGAAAAACACCCTGTTTCCTCTCCGGC CCCCACCGCGGATCATGTACCAGGATTATCCCGGGAACTTTGACACCTCGTCCCGGGG CAGCAGCGGCTCTCCTGCGCACGCCGAGTCCTACTCCAGCGGCGGCGGCGGCCAGC AGAAATTCCGGGTAGATATGCCTGGCTCAGGCAGTGCATTCATCCCCACCATCAACGC CATCACGACCAGCCAGGACCTGCAGTGGATGGTGCAGCCCACAGTGATCACCTCCAT GTCCAACCCATACCCTCGCTCGCACCCCTACAGCCCCCTGCCGGGCCTGGCCTCTGT CCCTGGACACATGGCCCTCCCAAGACCTGGCGTGATCAAGACCATTGGCACCACCGT GGGCCGCAGGAGGAGAGATGAGCAGCTGTCTCCTGAAGAGGAGGAGAAGCGTCGCAT CCGGCGGGAGAGGAACAAGCTGGCTGCAGCCAAGTGCCGGAACCGACGCCGGGAGC TGACAGAGAAGCTGCAGGCGGAGACAGAGGAGCTGGAGGAGGAGAAGTCAGGCCTG CAGAAGGAGATTGCTGAGCTGCAGAAGGAGAAGGAGAAGCTGGAGTTCATGTTGGTG GCTCACGGCCCAGTGTGCAAGATTAGCCCCGAGGAGCGCCGATCGCCCCCAGCCCCT GGGCTGCAGCCCATGCGCAGTGGGGGTGGCTCGGTGGGCGCTGTAGTGGTGAAACA GGAGCCCCTGGAAGAGGACAGCCCCTCGTCCTCGTCGGCGGGGCTGGACAAGGCCC AGCGCTCTGTCATCAAGCCCATCAGCATTGCTGGGGGCTTCTACGGTGAGGAGCCCCT GCACACCCCCATCGTGGTGACCTCCACACCTGCTGTCACTCCGGGCACCTCGAACCTC GTCTTCACCTATCCTAGCGTCCTGGAGCAGGAGTCACCCGCATCTCCCTCCGAATCCT GCTCCAAGGCTCACCGCAGAAGCAGTAGCAGCGGGGACCAATCATCAGACTCCTTGAA CTCCCCCACTCTGCTGGCTCTGTAACCCAGTGCACCTCCCTCCCCAGCTCCGGAGGG GGTCCTCCTCGCTCCTCCTTCCCAGGGACCAGCACCTTCAAGCGCTCCAGGGCCGTG AGGGCAAGAGGGGGACCTGCCACCAGGGAGCTTCCTGGCTCTGGGGGACCCAGGTG GGACTTAGCAGTGAGTATTGGAAGACTTGGGTTGATCTCTTAGAAGCCATGGGACCTC CTCCCTCATTCATCTTGCAAGCAAATCCCATTTCTTGAAAAGCCTTGGAGAACTCGGTTT GGTAGACTTGGACATCTCTCTGGCTTCTGAAGAGCCTGAAGCTGGCCTGGACCATTCC TGTCCCTTTGTTACCATACTGTCTCTGGAGTGATGGTGTCCTTCCCTGCCCCACCACGC ATGCTCAGTGCCTTTTGGTTTCACCTTCCCTCGACTTGACCCTTTCCTCCCCCAGCGTC AGTTTCACTCCCTCTTGGTTTTTATCAAATTTGCCATGACATTTCATCTGGGTGGTCTGA ATATTAAAGCTCTTCATTTCTGGAGATGGGGCAGCAGGTGGCTCTTCTGCTGGGGCTG ACTTGTCCAGAAGGGGACAAAGTGCAATACAGAGCCTTCCCTACCCTGACGCCTCCCA GTCATCATCTCCAGAACTCCCAGCGGGGCTCCCTGAGCTCTCAAGGAGATGCTGCCAT CACTGGGAGGCTCAGAGGACCCTTCCTGCCCACCTTCGGAGACGGCTTCTGGAGGAA CGGCTTGGCCAGAAGACAGGGTGTGAGTGAGACAGTGGGGCACAGGTTGGGTTTGCC AAACGCCTAATTACCAGGCCAGGAAGCATGCCAACAAAGCCACACGGGTGTCCTAGCC AGCTTCCCTTCACCTGGTGTCTTGAGTAGGGCGTCTCCTGTAATTACTGCCTTGCCATT CTGCCCCTGGACCCTTCTCTCCGGACCAGGGAGGCGTCCCTCCCTAGGAGCCACACA TTATACTCCAAGTCCCTGCCGGGCTCCGCCTTTCCCCCACCCTGGCTCTCAGGGTGAC GCCACCCACAGAGATTTAATGAGCGTGGGCCTGGACCTTCCCCAGATGCTGCCAGGC AGCCCCTCCCCAAGCCTCAAAGAAGCATTTGCTGAGGATGGAGAGGCAGGGGAGGGA GGCGGGAGGCCGTCACTGGAGTGGCGTCTGCAGCAGCTGCTGCCCCAGCACCCGCT CAGCCTGTCCTGGCTGCTCACCTCCCCGCAGGGCACCGGGCCTTTCCTGCCCTCTGT GGTCATCTGCCACCTGCTGGATCAAGTGCTTTCTCTTTTACACTCCCCTGTCCCCACCC CAGTGCACTCTTCTGGCCCAGGCAGCAAGCAAGCTGTGAACAGCTGGCCTGAGCTGT CGCTGTGGCTTGTGGCTCATGCGCCATTCCTGGTTGTCTGTTGAATCTTTCTGGCTGCT GGAATTGGAGATAGGATGTTTTGCTTCCCACTGCAGGAGAGCTGCCCCCTTTCACGGG GTTGGGGAAGGGTCCCCCTGGCCTCCAGCAGGAGCACAGCTCAGCAGGGTCCCTGCT GCCCACCCCTCTGAGCCTTTTCTCCCCAGGGTATGGCTCCTGCTGAGTTTCTTGTCCA GCAGGGCCTTGACAGGAATCCAGGGAGTAGCTCCTGGCCAGAACCAGCCTCTGCGGG GCTTGTGCTCTGCAAAGACTCTGCTGCTGGGGATTCAGCTCTAGAGGTCACAGTATCC TCGTTTGAAAGATAATTAGATCCCCCGTGGAGAAAGCAGTGACACATTCACACAGCTGT TCCCTCGCATGTTATTTCATGAACATGCCTGTTTTCGTGCACTAGACACACAGAGTGGA ACAGCCGTATGCTTAAAGTACATGGGCCAGTGGGACTGGAAGTGACCTGTACAAGTGA TGCAGAAAGGAGGGTTTCAAAGAAAAAGGATTTTGTTTAAAATACTTTAAAAATGTTATT TCCTGCATCCCTTGGCTGTGATGCCCCTCTCCCGATTTCCCAGGGGCTCTGGGAGGGA CCCTTCTAAGAAGATTGGGCAGTTGGGTTTCTGGCTTGAGATGAATCCAAGCAGCAGA ATGAGCCAGGAGTAGCAGGAGATGGGCAAAGAAAACTGGGGTGCACTCAGCTCTCAC AGGGGTAATCATCTCAAGTGGTATTTGTAGCCAAGTGGGAGCTATTTTCTTTTTTGTGC ATATAGATATTTCTTAAATGAAGCTGCTTTCTTGTCTTTTATTTCTAAAAGCCCCCTTATA CCCCACTTTGTGCAGCAAAGATCCCCGTGCAGGTCACAGCCTGATTTGTGGCCAGGCT GGACAAATTCCTGAGGCACAACTTGGCTTCAGTTCAGATTTCAAGCTGTGTTGGTGTTG GGACCAGCAGAAGGCAAACGTCCAGCCAACACACAGGACTGTAAGAGGACTCTGAGC TACGTGCCCTGTGAAGACCCCCAGGCTTTGTCATAGGAGGTCGTTCAGCTTCCCCAAA GTCAGAGGTGATTTGATTTGGGGAAGACTGAATATTCACACCTAAGTCGTGAGCATATC CTGAGTTTTACTTCCTTATGGCTTGCCCTCCAAGTTCTCTCTCTCATACACACACACACC CTTGCTCCAGAATCACCAGACACCTCCATGGCTCCAGCTATGGGAACAGCTGCATTGG GGCTGCCTTTCTGTTTGGCTTAGGAACTTCTGTGCTTCTTGTGGCTCCACTCGCGAGG CAGCTCGGAGGTGTGGACTCCGATTGGGCTGCAGGCAGCTCTGGGACGGCACAGGG CGGGCGCTCTGATCAGCTCGTGTAAAACACACCGTCTTCTTGGCCTCCTGGCCAGTCT TTCTGCGAATAGTCCTCTCCCTGGCCAGTTGAATGGGGGAAGCTGCTGGCACAGGAAG GAGAGGCGATCCCGGCTGAGGCTTAGGAAATTGCTGGAGCCGGCTCCAAGCAGATAA TTCACTGGGGAGGTTTTCAGAGTCAAACATCATTCTGCCTGTGTTGGGGGCCAGGTGT GTCACACAAGCATCTCAAAGTCAAAAGCCATCTGGGGCTGCTGCTTCTGTTTCTCAGGC TCTGGGGAAAGGAATCTCCCTCTCCTCTCACTTGATTCCAAGTGTGGTTGAATTGTCTG GAGCACTGGGACTTTTTTTCTCTTTTCCTTGATGGACCAACAGTGCAAATGCAATCTCG CCATTTAACTTTCAGGTCGATTTCCTTTCCTGATCAGACATCTTTGTGCCCCCTTTAGGA AGGAAAAGAATACACCTACGATGTGCCAGGCACTGTGTTAGGCGCTTTTATATAGATCC TCGTTAGGATGAGACTAAGGGATGAGGACATCTCTTTATAAAAGGCCCCTAAGTAATGG ATAAACAGAAACACTTAGAGGTGAGAAGGTCTGTCTTCAAGATCCAAGGTAAGATTGCC TTCAGTCTGATGTTTGTTCTCAAGGACTTATCCCCTACAATATTCTCCCACTCCATACTT CTCCTTCTACCCCACCATGTGCTCCCGTGCACTCCTCAGATGGTCAGAGGGGTAACCC AAGTCCTTAGAGAATTTGGGGACCAATAGAATATGTGATGTGTGAATTTTCTTTAAAAAA CTTAAGGAGTCTTTGCTACCTTCTGCTTGTTGAGTTGTTTTGGCATTCATATTAAAAGCC AGCATCTCACTATTTATTGACAGGTTGGGCTGTGTGTGTGCGCATGTGTGTATACATTT CCAGGCGTGCCTGTGTCCTGTAGCTTTTTAAAAGGAAACCCAGTCATCCCACTATGAAT CTGGCATCTTCTTATGCTTCTAGTGTTTTGGCCATACATCAACCAAGGGGTTTAATTTAT CCAATGCTTGACGACATGTTCAGGAGGGGCTGGATCAAATTTTGAGAGGGTTATGGGA AAGGGAGGGGGAGAAGAAATTGACATTTATTTTATTATTTATTTTAAATGTTTACATCTTC TTTATGTTGTATCAAGCCTGAATAGAAACTGATAGCATTAAAATACTCCGTTCCTCTCTC TCTTCTCGCTTCCTTTTTTTTTTTTTTTTTAAATTTAGGATAACACATTTTTGTTTCTAAAG TGATTTGTGATTTGTGCTGTATAAACTGTATAAAAGGTTCTGTTTTTAAAGGTGGATTTTC ATTCCTCTGGGGACAGTGGTCGCCAAGACATCTACATTGTAAGAGAACACAGTGGAAG ATCCTGTCCTGATTCTCAAAAATTATTTTCTCTGTATGATTAAAAGTTTATTCCATTTATTT TAGTTTGTGTTTACTTGATTTTGAGGAAGAAAATATTTGACTTTGTGTAAAGAGTAGGGT ATCAGGGTGTCTTTTCTGCCGTGGGAGATGTGTATATATATAGTATTTTGGTGTATAGTA GAAAATAAGCTTTGTGCATCTGTATTTGAGATATGTTAATGACGTGGAGTAAAGTCAGCT GTAAGACTCTGGAGGCAAACAAGTTGTATATGGTTCATATGGCTCTATGGGGAATTTAA TTACCTTTCTGGGCACTTTTTTTTTTTTTTTTTTTTTAAGTAATGGTGAAATGGTCCCATT GGAGAGTCTCCTAAATAGACCTTCCAGGCAGAACCGCAAGCTCAAAATCTTTGTATAGT TTTGAAAATTGAGGAGTAGCTTTGTTTGGAAGCCTTTCTGGTGGTGGTTTTTGTTGTTGT TGTTGTTTTGTTGTTTTACTATATGTAATACAAGCCTACAGTATTTGCACTAAAGAAAGCT TGTTAGAAAAAGCTTGCTGCTATGGAAGAAAGAACATATTAAAACTTCTTTCCCTTGCGA TTTTTTTGGGGGAGGGGGGTTAGCATTTCCACTTTCAGTTGAGTAGCATTTTGTAGAAT AAAATGAATTAAGATTGAAGAGCC. (SEQ ID NO:214; NM_005253.4), or a nucleic acid sequence that hybridizes to a nucleic acid sequence consisting of SEQ ID NO:214 under stringent hybridization conditions.

In some embodiments Jun proto-oncogene, AP-1 transcription factor subunit (JUN, p39, cJUN) comprises the amino acid sequence: MTAKMETTFYDDALNASFLPSESGPYGYSNPKILKQSMTLNLADPVGSLKPHLRAKNSDLL TSPDVGLLKLASPELERLIIQSSNGHITTTPTPTQFLCPKNVTDEQEGFAEGFVRALAELHSQ NTLPSVTSAAQPVNGAGMVAPAVASVAGGSGSGGFSASLHSEPPVYANLSNFNPGALSS GGGAPSYGAAGLAFPAQPQQQQQPPHHLPQQMPVQHPRLQALKEEPQTVPEMPGETPP LSPIDMESQERIKAERKRMRNRIAASKCRKRKLERIARLEEKVKTLKAQNSELASTANMLRE QVAQLKQKVMNHVNSGCQLMLTQQLQTF. (SEQ ID NO:215; N NP_002219.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:215)

In some embodiments, the nucleic acid sequence encoding JUN comprises the nucleic acid sequence: GCTCAGAGTTGCACTGAGTGTGGCTGAAGCAGCGAGGCGGGAGTGGAGGTGCGCGG AGTCAGGCAGACAGACAGACACAGCCAGCCAGCCAGGTCGGCAGTATAGTCCGAACT GCAAATCTTATTTTCTTTTCACCTTCTCTCTAACTGCCCAGAGCTAGCGCCTGTGGCTC CCGGGCTGGTGTTTCGGGAGTGTCCAGAGAGCCTGGTCTCCAGCCGCCCCCGGGAG GAGAGCCCTGCTGCCCAGGCGCTGTTGACAGCGGCGGAAAGCAGCGGTACCCACGC GCCCGCCGGGGGAAGTCGGCGAGCGGCTGCAGCAGCAAAGAACTTTCCCGGCTGGG AGGACCGGAGACAAGTGGCAGAGTCCCGGAGCCAACTTTTGCAAGCCTTTCCTGCGTC TTAGGCTTCTCCACGGCGGTAAAGACCAGAAGGCGGCGGAGAGCCACGCAAGAGAAG AAGGACGTGCGCTCAGCTTCGCTCGCACCGGTTGTTGAACTTGGGCGAGCGCGAGCC GCGGCTGCCGGGCGCCCCCTCCCCCTAGCAGCGGAGGAGGGGACAAGTCGTCGGAG TCCGGGCGGCCAAGACCCGCCGCCGGCCGGCCACTGCAGGGTCCGCACTGATCCGC TCCGCGGGGAGAGCCGCTGCTCTGGGAAGTGAGTTCGCCTGCGGACTCCGAGGAACC GCTGCGCACGAAGAGCGCTCAGTGAGTGACCGCGACTTTTCAAAGCCGGGTAGCGCG CGCGAGTCGACAAGTAAGAGTGCGGGAGGCATCTTAATTAACCCTGCGCTCCCTGGAG CGAGCTGGTGAGGAGGGCGCAGCGGGGACGACAGCCAGCGGGTGCGTGCGCTCTTA GAGAAACTTTCCCTGTCAAAGGCTCCGGGGGGCGCGGGTGTCCCCCGCTTGCCACAG CCCTGTTGCGGCCCCGAAACTTGTGCGCGCAGCCCAAACTAACCTCACGTGAAGTGAC GGACTGTTCTATGACTGCAAAGATGGAAACGACCTTCTATGACGATGCCCTCAACGCCT CGTTCCTCCCGTCCGAGAGCGGACCTTATGGCTACAGTAACCCCAAGATCCTGAAACA GAGCATGACCCTGAACCTGGCCGACCCAGTGGGGAGCCTGAAGCCGCACCTCCGCGC CAAGAACTCGGACCTCCTCACCTCGCCCGACGTGGGGCTGCTCAAGCTGGCGTCGCC CGAGCTGGAGCGCCTGATAATCCAGTCCAGCAACGGGCACATCACCACCACGCCGAC CCCCACCCAGTTCCTGTGCCCCAAGAACGTGACAGATGAGCAGGAGGGCTTCGCCGA GGGCTTCGTGCGCGCCCTGGCCGAACTGCACAGCCAGAACACGCTGCCCAGCGTCAC GTCGGCGGCGCAGCCGGTCAACGGGGCAGGCATGGTGGCTCCCGCGGTAGCCTCGG TGGCAGGGGGCAGCGGCAGCGGCGGCTTCAGCGCCAGCCTGCACAGCGAGCCGCCG GTCTACGCAAACCTCAGCAACTTCAACCCAGGCGCGCTGAGCAGCGGCGGCGGGGCG CCCTCCTACGGCGCGGCCGGCCTGGCCTTTCCCGCGCAACCCCAGCAGCAGCAGCA GCCGCCGCACCACCTGCCCCAGCAGATGCCCGTGCAGCACCCGCGGCTGCAGGCCC TGAAGGAGGAGCCTCAGACAGTGCCCGAGATGCCCGGCGAGACACCGCCCCTGTCCC CCATCGACATGGAGTCCCAGGAGCGGATCAAGGCGGAGAGGAAGCGCATGAGGAACC GCATCGCTGCCTCCAAGTGCCGAAAAAGGAAGCTGGAGAGAATCGCCCGGCTGGAGG AAAAAGTGAAAACCTTGAAAGCTCAGAACTCGGAGCTGGCGTCCACGGCCAACATGCT CAGGGAACAGGTGGCACAGCTTAAACAGAAAGTCATGAACCACGTTAACAGTGGGTGC CAACTCATGCTAACGCAGCAGTTGCAAACATTTTGAAGAGAGACCGTCGGGGGCTGAG GGGCAACGAAGAAAAAAAATAACACAGAGAGACAGACTTGAGAACTTGACAAGTTGCG ACGGAGAGAAAAAAGAAGTGTCCGAGAACTAAAGCCAAGGGTATCCAAGTTGGACTGG GTTGCGTCCTGACGGCGCCCCCAGTGTGCACGAGTGGGAAGGACTTGGCGCGCCCTC CCTTGGCGTGGAGCCAGGGAGCGGCCGCCTGCGGGCTGCCCCGCTTTGCGGACGGG CTGTCCCCGCGCGAACGGAACGTTGGACTTTTCGTTAACATTGACCAAGAACTGCATG GACCTAACATTCGATCTCATTCAGTATTAAAGGGGGGAGGGGGAGGGGGTTACAAACT GCAATAGAGACTGTAGATTGCTTCTGTAGTACTCCTTAAGAACACAAAGCGGGGGGAG GGTTGGGGAGGGGCGGCAGGAGGGAGGTTTGTGAGAGCGAGGCTGAGCCTACAGAT GAACTCTTTCTGGCCTGCCTTCGTTAACTGTGTATGTACATATATATATTTTTTAATTTGA TGAAAGCTGATTACTGTCAATAAACAGCTTCATGCCTTTGTAAGTTATTTCTTGTTTGTTT GTTTGGGTATCCTGCCCAGTGTTGTTTGTAAATAAGAGATTTGGAGCACTCTGAGTTTA CCATTTGTAATAAAGTATATAATTTTTTTATGTTTTGTTTCTGAAAATTCCAGAAAGGATA TTTAAGAAAATACAATAAACTATTGGAAAGTACTCCCCTAACCTCTTTTCTGCATCATCT GTAGATACTAGCTATCTAGGTGGAGTTGAAAGAGTTAAGAATGTCGATTAAAATCACTC TCAGTGCTTCTTACTATTAAGCAGTAAAAACTGTTCTCTATTAGACTTTAGAAATAAATGT ACCTGATGTACCTGATGCTATGGTCAGGTTATACTCCTCCTCCCCCAGCTATCTATATG GAATTGCTTACCAAAGGATAGTGCGATGTTTCAGGAGGCTGGAGGAAGGGGGGTTGCA GTGGAGAGGGACAGCCCACTGAGAAGTCAAACATTTCAAAGTTTGGATTGTATCAAGT GGCATGTGCTGTGACCATTTATAATGTTAGTAGAAATTTTACAATAGGTGCTTATTCTCA AAGCAGGAATTGGTGGCAGATTTTACAAAAGATGTATCCTTCCAATTTGGAATCTTCTCT TTGACAATTCCTAGATAAAAAGATGGCCTTTGCTTATGAATATTTATAACAGCATTCTTGT CACAATAAATGTATTCAAATACCAA. (SEQ ID NO:216; NM_002228.4), or a nucleic acid sequence that hybridizes to a nucleic acid sequence consisting of SEQ ID NO:216 under stringent hybridization conditions.

In some embodiments, JunB proto-oncogene, AP-1 transcription factor subunit (JUNB) comprises the amino acid sequence: MCTKMEQPFYHDDSYTATGYGRAPGGLSLHDYKLLKPSLAVNLADPYRSLKAPGARGPGP EGGGGGSYFSGQGSDTGASLKLASSELERLIVPNSNGVITTTPTPPGQYFYPRGGGSGGG AGGAGGGVTEEQEGFADGFVKALDDLHKMNHVTPPNVSLGATGGPPAGPGGVYAGPEP PPVYTNLSSYSPASASSGGAGAAVGTGSSYPTTTISYLPHAPPFAGGHPAQLGLGRGASTF KEEPQTVPEARSRDATPPVSPINMEDQERIKVERKRLRNRLAATKCRKRKLERIARLEDKVK TLKAENAGLSSTAGLLREQVAQLKQKVMTHVSNGCQLLLGVKGHAF (SEQ ID NO:217; NP_002220.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:217)

In some embodiments, the nucleic acid sequence encoding JUNB comprises the nucleic acid sequence: GGGACCTTGAGAGCGGCCAGGCCAGCCTCGGAGCCAGCAGGGAGCTGGGAGCTGGG GGAAACGACGCCAGGAAAGCTATCGCGCCAGAGAGGGCGACGGGGGCTCGGGAAGC CTGACAGGGCTTTTGCGCACAGCTGCCGGCTGGCTGCTACCCGCCCGCGCCAGCCCC CGAGAACGCGCGACCAGGCACCCAGTCCGGTCACCGCAGCGGAGAGCTCGCCGCTC GCTGCAGCGAGGCCCGGAGCGGCCCCGCAGGGACCCTCCCCAGACCGCCTGGGCCG CCCGGATGTGCACTAAAATGGAACAGCCCTTCTACCACGACGACTCATACACAGCTAC GGGATACGGCCGGGCCCCTGGTGGCCTCTCTCTACACGACTACAAACTCCTGAAACC GAGCCTGGCGGTCAACCTGGCCGACCCCTACCGGAGTCTCAAAGCGCCTGGGGCTCG CGGACCCGGCCCAGAGGGCGGCGGTGGCGGCAGCTACTTTTCTGGTCAGGGCTCGG ACACCGGCGCGTCTCTCAAGCTCGCCTCTTCGGAGCTGGAACGCCTGATTGTCCCCAA CAGCAACGGCGTGATCACGACGACGCCTACACCCCCGGGACAGTACTTTTACCCCCG CGGGGGTGGCAGCGGTGGAGGTGCAGGGGGCGCAGGGGGCGGCGTCACCGAGGAG CAGGAGGGCTTCGCCGACGGCTTTGTCAAAGCCCTGGACGATCTGCACAAGATGAAC CACGTGACACCCCCCAACGTGTCCCTGGGCGCTACCGGGGGGCCCCCGGCTGGGCC CGGGGGCGTCTACGCCGGCCCGGAGCCACCTCCCGTTTACACCAACCTCAGCAGCTA CTCCCCAGCCTCTGCGTCCTCGGGAGGCGCCGGGGCTGCCGTCGGGACCGGGAGCT CGTACCCGACGACCACCATCAGCTACCTCCCACACGCGCCGCCCTTCGCCGGTGGCC ACCCGGCGCAGCTGGGCTTGGGCCGCGGCGCCTCCACCTTCAAGGAGGAACCGCAG ACCGTGCCGGAGGCGCGCAGCCGGGACGCCACGCCGCCGGTGTCCCCCATCAACAT GGAAGACCAAGAGCGCATCAAAGTGGAGCGCAAGCGGCTGCGGAACCGGCTGGCGG CCACCAAGTGCCGGAAGCGGAAGCTGGAGCGCATCGCGCGCCTGGAGGACAAGGTG AAGACGCTCAAGGCCGAGAACGCGGGGCTGTCGAGTACCGCCGGCCTCCTCCGGGA GCAGGTGGCCCAGCTCAAACAGAAGGTCATGACCCACGTCAGCAACGGCTGTCAGCT GCTGCTTGGGGTCAAGGGACACGCCTTCTGAACGTCCCCTGCCCCTTTACGGACACCC CCTCGCTTGGACGGCTGGGCACACGCCTCCCACTGGGGTCCAGGGAGCAGGCGGTG GGCACCCACCCTGGGACCTAGGGGCGCCGCAAACCACACTGGACTCCGGCCCTCCTA CCCTGCGCCCAGTCCTTCCACCTCGACGTTTACAAGCCCCCCCTTCCACTTTTTTTTGT ATGTTTTTTTTCTGCTGGAAACAGACTCGATTCATATTGAATATAATATATTTGTGTATTT AACAGGGAGGGGAAGAGGGGGCGATCGCGGCGGAGCTGGCCCCGCCGCCTGGTACT CAAGCCCGCGGGGACATTGGGAAGGGGACCCCCGCCCCCTGCCCTCCCCTCTCTGCA CCGTACTGTGGAAAAGAAACACGCACTTAGTCTCTAAAGAGTTTATTTTAAGACGTGTTT GTGTTTGTGTGTGTTTGTTCTTTTTATTGAATCTATTTAAGTAAAAAAAAAATTGGTTCTT TATTAA. (SEQ ID NO:218; NM_002229.3), or a nucleic acid sequence that hybridizes to a nucleic acid sequence consisting of SEQ ID NO:218 under stringent hybridization conditions.

In some embodiments, JunD proto-oncogene, AP-1 transcription factor subunit (JunD) comprises the amino acid sequence: MMKKDALTLSLSEQVAAALKPAAAPPPTPLRADGAPSAAPPDGLLASPDLGLLKLASPELE RLIIQSNGLVTTTPTSSQFLYPKVAASEEQEFAEGFVKALEDLHKQNQLGAGAAAAAAAAAA GGPSGTATGSAPPGELAPAAAAPEAPVYANLSSYAGGAGGAGGAATVAFAAEPVPFPPPP PPGALGPPRLAALKDEPQTVPDVPSFGESPPLSPIDMDTQERIKAERKRLRNRIAASKCRK RKLERISRLEEKVKTLKSQNTELASTASLLREQVAQLKQKVLSHVNSGCQLLPQHQVPAY. (SEQ ID NO:219; NP_001273897.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:219)

In some embodiments, the nucleic acid sequence encoding JUND comprises the nucleic acid sequence: AGGAGCCGCCGCCAGTGGAGGGCCGGGCGCTGCGGCCGCGGCCGGGGCGGGCGCA GGGCCGAGCGGACGGGGGGGCGCGGGCCCCCCGGGAGGCCGCGGCCACTCCCCCC CGGGCCGGCGCGGCGGGGGAGGCGGAGGATGGAAACACCCTTCTACGGCGATGAGG CGCTGAGCGGCCTGGGCGGCGGCGCCAGTGGCAGCGGCGGCAGCTTCGCGTCCCC GGGCCGCTTGTTCCCCGGGGCGCCCCCGACGGCCGCGGCCGGCAGCATGATGAAGA AGGACGCGCTGACGCTGAGCCTGAGTGAGCAGGTGGCGGCAGCGCTCAAGCCTGCG GCCGCGCCGCCTCCTACCCCCCTGCGCGCCGACGGCGCCCCCAGCGCGGCACCCCC CGACGGCCTGCTCGCCTCTCCCGACCTGGGGCTGCTGAAGCTGGCCTCCCCCGAGCT CGAGCGCCTCATCATCCAGTCCAACGGGCTGGTCACCACCACGCCGACGAGCTCACA GTTCCTCTACCCCAAGGTGGCGGCCAGCGAGGAGCAGGAGTTCGCCGAGGGCTTCGT CAAGGCCCTGGAGGATTTACACAAGCAGAACCAGCTCGGCGCGGGCGCGGCCGCTG CCGCCGCCGCCGCCGCCGCCGGGGGGCCCTCGGGCACGGCCACGGGCTCCGCGCC CCCCGGCGAGCTGGCCCCGGCGGCGGCCGCGCCCGAAGCGCCTGTCTACGCGAACC TGAGCAGCTACGCGGGCGGCGCCGGGGGCGCGGGGGGCGCCGCGACGGTCGCCTT CGCTGCCGAACCTGTGCCCTTCCCGCCGCCGCCACCCCCAGGCGCGTTGGGGCCGC CGCGCCTGGCTGCGCTCAAGGACGAGCCACAGACGGTGCCCGACGTGCCGAGCTTC GGCGAGAGCCCGCCGTTGTCGCCCATCGACATGGACACGCAGGAGCGCATCAAGGC GGAGCGCAAGCGGCTGCGCAACCGCATCGCCGCCTCCAAGTGCCGCAAGCGCAAGC TGGAGCGCATCTCGCGCCTGGAAGAGAAAGTGAAGACCCTCAAGAGTCAGAACACGG AGCTGGCGTCCACGGCGAGCCTGCTGCGCGAGCAGGTGGCGCAGCTCAAGCAGAAA GTCCTCAGCCACGTCAACAGCGGCTGCCAGCTGCTGCCCCAGCACCAGGTGCCCGCG TACTGAGTCCGCGCGCGGGGCGCATGCGCGGCCACCCTCCCCAAGGGGCGGGCTCG CGGGGGGGTGTCGTGGGCGCCCCGGACTTGGAGAGGGTGCGGCCCTGGGGACCCC CCCTCCCCGAGTGTGCCCAGGAACTCAGAGAGGGCGCGGCCCCCGGGGATTCCCCC CCCCCGAGGGTGCCCAGGACTCGACAAGCTGGACCCCCTGCTCCCGGGGGGGCGAG CGCATGACCCCCCCGCCCTCGCGCTGCCTCTTTCCCCCGCGCGGCCGCCCCGTGTTG CACAAACCCGCGCGTCTCGGCTGCCCCTTTGTACACCGCGCCGCGGAAGGGGGCTCC GAGGGGGCGCAGCCTCAAACCCTGCCTTTCCTTTACTTTTACTTTTTTTTTTTTTTCTTT GGAAGAGAGAAGAACAGAGTGTTCGATTCTGCCCTATTTATGTTTCTACTCGGGAACAA ACGTTGGTTGTGTGTGTGTGTGTTTTCTTGTGTTGGTTTTTTAAAGAAATGGGAAGAAGA AAAAAAAATTCTCCGCCCCTTTCCTCGATCTCGCTCCCCCCTTCGGTTCTTTCGACCGG TCCCCCCTCCCTTTTTTGTTCTGTTTTGTTTTGTTTTGCTACGAGTCCACATTCCTGTTTG TAATCCTTGGTTCGCCCGGTTTTCTGTTTTCAGTAAAGTCTCGTTACGCCAGCTCGGCT CTCCGCCTCCTTCTTCCCCCGCCGGGGCCTGGCGGGCTGGGCGGGGCCTGGTTCGC TT. (SEQ ID NO:220; NM_001286968.2), or a nucleic acid sequence that hybridizes to a nucleic acid sequence consisting of SEQ ID NO:220 under stringent hybridization conditions.

In some embodiments, ZFP36 ring finger protein (ZFP36, TTP) comprises the amino acid sequence: MDLTAIYESLLSLSPDVPVPSDHGGTESSPGWGSSGPWSLSPSDSSPSGVTSRLPGRSTS LVEGRSCGWVPPPPGFAPLAPRLGPELSPSPTSPTATSTTPSRYKTELCRTFSESGRCRY GAKCQFAHGLGELRQANRHPKYKTELCHKFYLQGRCPYGSRCHFIHNPSEDLAAPGHPPV LRQSISFSGLPSGRRTSPPPPGLAGPSLSSSSFSPSSSPPPPGDLPLSPSAFSAAPGTPLA RRDPTPVCCPSCRRATPISVWGPLGGLVRTPSVQSLGSDPDEYASSGSSLGGSDSPVFEA GVFAPPQPVAAPRRLPIFNRISVSE. (SEQ ID NO:221; NP_00339.3), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:221)

In some embodiments, the nucleic acid sequence encoding ZFP36 comprises the nucleic acid sequence: AGCCTGACTTCAGCGCTCCCACTCTCGGCCGACACCCCTCATGGCCAACCGTTACACC ATGGATCTGACTGCCATCTACGAGAGCCTCCTGTCGCTGAGCCCTGACGTGCCCGTGC CATCCGACCATGGAGGGACTGAGTCCAGCCCAGGCTGGGGCTCCTCGGGACCCTGGA GCCTGAGCCCCTCCGACTCCAGCCCGTCTGGGGTCACCTCCCGCCTGCCTGGCCGCT CCACCAGCCTAGTGGAGGGCCGCAGCTGTGGCTGGGTGCCCCCACCCCCTGGCTTCG CACCGCTGGCTCCCCGCCTGGGCCCTGAGCTGTCACCCTCACCCACTTCGCCCACTG CAACCTCCACCACCCCCTCGCGCTACAAGACTGAGCTATGTCGGACCTTCTCAGAGAG TGGGCGCTGCCGCTACGGGGCCAAGTGCCAGTTTGCCCATGGCCTGGGCGAGCTGC GCCAGGCCAATCGCCACCCCAAATACAAGACGGAACTCTGTCACAAGTTCTACCTCCA GGGCCGCTGCCCCTACGGCTCTCGCTGCCACTTCATCCACAACCCTAGCGAAGACCT GGCGGCCCCGGGCCACCCTCCTGTGCTTCGCCAGAGCATCAGCTTCTCCGGCCTGCC CTCTGGCCGCCGGACCTCACCACCACCACCAGGCCTGGCCGGCCCTTCCCTGTCCTC CAGCTCCTTCTCGCCCTCCAGCTCCCCACCACCACCTGGGGACCTTCCACTGTCACCC TCTGCCTTCTCTGCTGCCCCTGGCACCCCCCTGGCTCGAAGAGACCCCACCCCAGTCT GTTGCCCCTCCTGCCGAAGGGCCACTCCTATCAGCGTCTGGGGGCCCTTGGGTGGCC TGGTTCGGACCCCCTCTGTACAGTCCCTGGGATCCGACCCTGATGAATATGCCAGCAG CGGCAGCAGCCTGGGGGGCTCTGACTCTCCCGTCTTCGAGGCGGGAGTTTTTGCACC ACCCCAGCCCGTGGCAGCCCCCCGGCGACTCCCCATCTTCAATCGCATCTCTGTTTCT GAGTGACAAAGTGACTGCCCGGTCAGATCAGCTGGATCTCAGCGGGGAGCCACGTCT CTTGCACTGTGGTCTCTGCATGGACCCCAGGGCTGTGGGGACTTGGGGGACAGTAAT CAAGTAATCCCCTTTTCCAGAATGCATTAACCCACTCCCCTGACCTCACGCTGGGGCA GGTCCCCAAGTGTGCAAGCTCAGTATTCATGATGGTGGGGGATGGAGTGTCTTCCGAG GTTCTTGGGGGAAAAAAAATTGTAGCATATTTAAGGGAGGCAATGAACCCTCTCCCCCA CCTCTTCCCTGCCCAAATCTGTCTCCTAGAATCTTATGTGCTGTGAATAATAGGCCTTCA CTGCCCCTCCAGTTTTTATAGACCTGAGGTTCCAGTGTCTCCTGGTAACTGGAACCTCT CCTGAGGGGGAATCCTGGTGCTCAAATTACCCTCCAAAAGCAAGTAGCCAAAGCCGTT GCCAAACCCCACCCATAAATCAATGGGCCCTTTATTTATGACGACTTTATTTATTCTAAT ATGATTTTATAGTATTTATATATATTGGGTCGTCTGCTTCCCTTGTATTTTTCTTCCTTTTT TTGTAATATTGAAAACGACGATATAATTATTATAAGTAGACTATAATATATTTAGTAATAT ATATTATTACCTTAAAAGTCTATTTTTGTGTTTTGGGCATTTTTAAATAAACAATCTGAGT GTAA. (SEQ ID NO:222; NM_003407.5), or a nucleic acid sequence that hybridizes to a nucleic acid sequence consisting of SEQ ID NO:222 under stringent hybridization conditions.

In some embodiments, EBF transcription factor 1 (EBF1, EBF, COE1, OL1) ) comprises the amino acid sequence: MFGIQESIQRSGSSMKEEPLGSGMNAVRTWMQGAGVLDANTAAQSGVGLARAHFEKQPP SNLRKSNFFHFVLALYDRQGQPVEIERTAFVGFVEKEKEANSEKTNNGIHYRLQLLYSNGIR TEQDFYVRLIDSMTKQAIVYEGQDKNPEMCRVLLTHEIMCSRCCDKKSCGNRNETPSDPVII DRFFLKFFLKCNQNCLKNAGNPRDMRRFQVVVSTTVNVDGHVLAVSDNMFVHNNSKHGR RARRLDPSEGTPSYLEHAATPCIKAISPSEGWTTGGATVIIIGDNFFDGLQVIFGTMLVWSEL ITPHAIRVQTPPRHIPGVVEVTLSYKSKQFCKGTPGRFIYTALNEPTIDYGFQRLQKVIPRHP GDPERLPKEVILKRAADLVEALYGMPHNNQEIILKRAADIAEALYSVPRNHNQLPALANTSV HAGMMGVNSFSGQLAVNVSEASQATNQGFTRNSSSVSPHGYVPSTTPQQTNYNSVTTSM NGYGSAAMSNLGGSPTFLNGSAANSPYAIVPSSPTMASSTSLPSNCSSSSGIFSFSPANMV SAVKQKSAFAPVVRPQTSPPPTCTSTNGNSLQAISGMIVPPM. (SEQ ID NO:223; NP_001277289.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:223)

In some embodiments, the nucleic acid sequence encoding EBF1 comprises the nucleic acid sequence: CCTGCTTCTTCAAGTGAAGGGTACCTCTACAAAAGGAAACTCCAGCCCCTCCTGTCCTC CACCGGCCTGTGATCATTACAAAAAAAAAAAAAAAAAAGCAAAAAAAAAAAAAAAGCAC CCAAACCAAAAATCAACCAACCAAACAACCCCCAACAGCCAAGCATACATCTCTAATTT TATTATTTTGGTCTTTTCGTTGGATTTTCCCTTTCTTCTTTTTTTCGGGTTATCGCTCAGT TTTGAGCAGAGGTTTACATTTTTTAAAAATTTGCTTTCCAGCCCGCCTTGATCTTCTAAG TGCGAGTTCATCGTCTGAGAAAAAAAAAAATCTCTGGTTGGCGTTTTTGTTTCTTTTCTT TTCTTTCTTTTCTTTCCTTTTTTTTTTTTTTTAATTTTTTTCAAGGGGGAGGAGATTTTCCA CAAGAAAAGGTTGTTTTCATGTTTGGGATTCAGGAAAGCATCCAACGGAGTGGAAGCA GCATGAAGGAAGAGCCGCTGGGCAGCGGCATGAACGCGGTGCGGACGTGGATGCAG GGCGCCGGGGTGCTGGACGCCAACACGGCGGCGCAGAGCGGGGTGGGTCTGGCCC GGGCTCACTTTGAGAAGCAGCCGCCTTCCAATCTGCGGAAATCCAACTTCTTCCACTTC GTCCTGGCCCTCTACGACAGACAGGGCCAGCCCGTGGAGATCGAGAGGACAGCGTTT GTGGGGTTCGTGGAGAAGGAAAAAGAAGCCAACAGCGAAAAGACCAATAACGGAATTC ACTACCGGCTTCAGCTTCTCTACAGCAATGGGATAAGGACGGAGCAGGATTTCTACGT GCGCCTCATTGACTCCATGACAAAACAAGCCATAGTGTATGAAGGCCAAGACAAGAAC CCAGAAATGTGCCGAGTCTTGCTCACACATGAGATCATGTGCAGCCGCTGTTGTGACA AGAAAAGCTGTGGCAACCGAAATGAGACTCCCTCAGATCCAGTGATAATTGACAGGTT CTTCTTGAAATTTTTCCTCAAATGTAACCAAAATTGCCTAAAGAATGCGGGAAACCCACG TGACATGCGGAGATTCCAGGTCGTGGTGTCTACGACAGTCAATGTGGATGGCCATGTC CTGGCAGTCTCTGATAACATGTTTGTCCATAATAATTCCAAGCATGGGCGGAGGGCTC GGAGGCTTGACCCCTCGGAAGGTACGCCCTCTTATCTGGAACATGCAGCTACTCCCTG TATCAAAGCCATCAGCCCGAGTGAAGGATGGACGACGGGAGGTGCGACTGTGATCAT CATAGGGGACAATTTCTTTGATGGGTTACAGGTCATATTCGGTACCATGCTGGTCTGGA GTGAGTTGATCACTCCTCATGCCATCCGTGTGCAGACCCCTCCTCGGCACATCCCTGG TGTTGTGGAAGTCACACTGTCCTACAAATCTAAGCAGTTCTGCAAAGGAACACCAGGCA GATTCATTTATACAGCGCTCAACGAACCCACCATCGATTATGGTTTCCAGAGGTTACAG AAGGTCATTCCTCGGCACCCTGGTGACCCTGAGCGTTTGCCAAAGGAAGTAATACTCA AAAGGGCTGCGGATCTGGTAGAAGCACTGTATGGGATGCCACACAACAACCAGGAAAT CATTCTGAAGAGAGCGGCCGACATTGCCGAGGCCCTGTACAGTGTTCCCCGCAACCAC AACCAACTCCCGGCCCTTGCTAACACCTCGGTCCACGCAGGGATGATGGGCGTGAATT CGTTCAGTGGACAACTGGCCGTGAATGTCTCCGAGGCATCACAAGCCACCAATCAGGG TTTCACCCGCAACTCAAGCAGCGTATCACCACACGGGTACGTGCCGAGCACCACTCCC CAGCAGACCAACTATAACTCCGTCACCACGAGCATGAACGGATACGGCTCTGCCGCAA TGTCCAATTTGGGCGGCTCCCCCACCTTCCTCAACGGCTCAGCTGCCAACTCCCCCTA TGCCATAGTGCCATCCAGCCCCACCATGGCCTCCTCCACAAGCCTCCCCTCCAACTGC AGCAGCTCCTCGGGCATCTTCTCCTTCTCACCAGCCAACATGGTCTCAGCCGTGAAAC AGAAGAGTGCTTTCGCACCAGTCGTCAGACCCCAGACCTCCCCACCTCCCACCTGCAC CAGCACCAACGGGAACAGCCTGCAAGCGATATCTGGCATGATTGTTCCTCCTATGTGA AAGAATTGCCTTGAAGAATTGTATTAATGAAGAGGTTGGATTCTGCTACAGAGAGTAAT CTGATACAAGTCCCAGAGTGGAACTTTTAACTCAGGCCTTTTTAAGAGGAATCACAATA ACTGCAGATTTTTAAACAAACAAAATCACCGACCTTGCAAATACTGAAATTGGAAGAGG GATCTGCAAGTGCAGGGTGTTGGTTAAAGTTGTACCTCCCAAGTATTTGGGGGATATAT TTATTCTGTATTGACAAAAGCAAATCCACTTTTTCTTTTTCTTTTTTTTTTTTAAGCTTAAT TCTGCAATCATTTGTCTTTTATAAACCGTAAAGCTCTATACAAGGGACACTATAAATAAG ACTCCATGTTTTAATTTATGATGTTTTTAAAGCTGTGTAAAGGAAGAATGAAGTGGTGAT ATTTACAAAAAAGTAAAAAAAAAAAAAAAGAAAAAAAGGAAAAAAAAAAAAGCTTGTATG GGACAGAATAGGAATGCCAGTTAGATTTTTTAGAAAAACTAAGGGTCGGCTTTTGCGCC TTAAAGCATATCAAATGGTAGTTAGCTCAGACAGTGCATTTTCAATATCTAACTTAACAT GCCACCCCTTAGCAGTGCAAGCTTATTTATCTCTTTTGTATTGTTGTCTTAAGCAACTGT GTAAATAAATGCAGCCTGGAAAGTTAAAACGCGATGTAAGTGATCACATTTTTCCCTCTA CTCGAAAATCCAGTGCCTCTAGACAGATTGTTAAAACTGCATATTTAAATCTGATCTCAT TCTCTCCTTTTACTTAAGTCAGTTTCTTCTGAAGCCGATGGCCTCTCGAGAGCTTGGTG GCACACATACTGTGTGAGAATCTCCTTTGAGACTTCATGGAACAGGGTCCAGGCACAG AATTCCACACTCTGCCCTCCAGTTAACAAGCCAAAACCTCACGTACGCTCCCCCATTTG CAAGTTTAAAGTTTACTCGTCCTAAATGCAGACTTCATACCTATCTTCCAAAAGTTGATA AAAGCAACGTGGCAGGTATTTTCGATTTTCCCATTCCAGATACTGCCTCTATCAGTCGA CCCTTACCATTTGTAACATAATAGATTGAAACAACAGGTTAAGTGCTTTGGAATTAAGAG TTAAAGGGAACCGGGGGTGGAGAAAGGAAAAGAAGGTTGAGACCTCCAAAACATAGTT TTCTGTTCTATGGGAATTTTTGCTCTCATTATCTGGGAAGTGTTCTTAAAAATAAGAATTA ATAGCAAAGATGCAGCAAAGCTCTGAGGATGCATTTGCCTGATATTTTTTTCTTTGCTCT TGTGTTTTTGTATGTAGTTATAAATACTGTAGATTTTTTTTTTGTGATTTTTTGCCAAAGTT GTTGTTCTATTTATACATTTTAATGTCTTAAGACATTTTTTCAATATCACAAAAAGATTTTA CTGCGTATTTTGCAAAGAAAAAAAGCTCACTACCTTTAGCTTGCACATACTTGCAAAGTT AATTAAAAGGCTTTTTGTTTTAAAGGGGATTTTGTAAAATATCCATATAAATAATGTATTT ATCTTTCGAATTTGTACATTGCTTTTCCCTTCTTCCTCTTCCTTCCACCCCCAATTTATTT TATTGTGTATGTTTGCTACGTGAAAAGTGCGTATTTGTTTGGTCACCTACACTTGTATTA GCTGTTTCAATGTGATTTTTAAACATTTCATTTATAGTTATTTTTAGTATTGTTATAAACCA TGCTTCAGTTTTTTTAATTTCCACCCAAAAGTCATTGTCTATTTTTGTATTATTTGTAAGTT AAGAAGTTTTTTCCAATATATGGCAAAAAAAAAAAAATAGTAGCATATTATTCTTGTAGTA TTTAGTTCAGTAGATTTAAAAAAAAATGTATCCTTTGCTTTGGAAGCTTACAAAACAACC CTAATGCTGTTTTACTCTATTAATATGCATGGAATCTCTCCCTTTGGAGTGACGCATTTT GTGCATTAAATTCTAGGGAGAAACTTCATAGAAATCAATGAACATACTTTCTTTCTTAAG TCTGCTTGTATATTTCCTCTGTCTTTCACATAAATATAAACCAGCAGATTGGATGCCTTA ACAATGCAAATCATATTCATTTCACTTGTACATTGTAACTGTGCACCAGAACTGTCAGTC ATCACTAACATTCTAAGAAAAAAGAAAAAGAAAAAAAGAAAAAAAAAAAAACAAAGAAAT CGAAAAGCACAAAAGAACTGTTTTGTTACCTTAAGACAATGTAACTTTTTCTAGTAGAGC AAGAAATCATTTACAACAATGCTGCAACTGTGCATGCCCCCATATGGATTTTGCAATGG TTTTCACTAGGCTGTCAAGAGTGCGATTTTTATGGGTTGGGGTGGGTGGGGGAGGGGA GTTGTGGGAGGGTAGGGGAGGGAGGAAAGCTGTTTTTCATGGTGAGAAATAATAATGA TGACTAATAATAATAAAAAAAACTGGAAAATGTAAGCAAGGTGGACGCATTGCTTCTCG GTACTCAGAAAGGTTATCTGAATTTGCGTGGTAAGCGCTGGCCTGAAGATGTACACAA GTTAAAGCCATATTTTATCTGGTGAGCCCCTTAACTGTTTCTGAAGGAATGAACGGTCA GCCGGGAAGGTGTCCGGCTTAGACCTTGACAACAGACACTACCCATCTGTCAGCCATG TGCAGTGGTTAGAACTCTTCTTGAAAGTCCAAAGAGCCTTTAAAATGTGTATAATTTGTG TTTTGTTGCTTCTATTTCTATTGATTAGATGAAAAGACATTCTGGCCCTCTGATCCTCTTT CTTCTCCACAAAAGTTTTACAAATAAAAGATGTTCCCCTAAATACAGAATATGGCTTTAA GAAGAAAATGAAATGAAGAATTAAATTAAGATTTCAGTGTTGGGGGAAAAAATACAGCTT CTGGATGACTGGATTGCATAACATTGCCCTGGCCTCACATTGTACTAGGATGCAGCTTA AATGAAGTCATCTCTAAATCTACTAACCTTTCACCTTCTTATCAAAACTTTTTGAATAGAC ACACACTGTACAGTTCAATTGTTAGAGAACCTAACTACTGTAGAGATTGTTAAATTTTTTT TTTTTTTTTGCAAAAATTCAAGCTGTAAAAACTTTTCAACTTTCACAATATTTAATTAAAGT TACTTCCTGTCTGTGA. (SEQ ID NO:224; NM_001290360.3), or a nucleic acid sequence that hybridizes to a nucleic acid sequence consisting of SEQ ID NO:224 under stringent hybridization conditions.

In some embodiments, EBF transcription factor 3 (EBF3, COE3, OE2, HADDS) comprises the amino acid sequence: MFGIQENIPRGGTTMKEEPLGSGMNPVRSWMHTAGVVDANTAAQSGVGLARAHFEKQPP SNLRKSNFFHFVLALYDRQGQPVEIERTAFVDFVEKEKEPNNEKTNNGIHYKLQLLYSNGV RTEQDLYVRLIDSMTKQAIVYEGQDKNPEMCRVLLTHEIMCSRCCDKKSCGNRNETPSDP VIIDRFFLKFFLKCNQNCLKNAGNPRDMRRFQVVVSTTVNVDGHVLAVSDNMFVHNNSKH GRRARRLDPSEATPCIKAISPSEGWTTGGATVIIIGDNFFDGLQVVFGTMLVWSELITPHAIR VQTPPRHIPGVVEVTLSYKSKQFCKGAPGRFVYTALNEPTIDYGFQRLQKVIPRHPGDPER LPKEVLLKRAADLVEALYGMPHNNQEIILKRAADIAEALYSVPRNHNQIPTLGNNPAHTGMM GVNSFSSQLAVNVSETSQANDQVGYSRNTSSVSPRGYVPSSTPQQSNYNTVSTSMNGYG SGAMASLGVPGSPGFLNGSSANSPYGMKQKSAFAPVVRPQASPPPSCTSANGNGLQAMS GLVVPPM. (SEQ ID NO:225; NP_001005463.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:225)

In some embodiments, the nucleic acid sequence encoding EBF3 comprises the nucleic acid sequence: ATTCCTCGGGGAGAGAGGGCTGAGTTTTGTGCGCCTCCGTCCGCTGCGCGCGCCGCT CCAGGCCCCGCCGCGCCCCGCCTGCGCCCGGGACTGCGCCGCAGCACTCACTGCCC CGTTTATTTGTCTTTGGAGCAGGCGGGCCGCGCCAGAAGCGGGCGATCCCGCAGTGT CCTGCAGCCGCGGACGCCGCCTGGCTAGGATGACACCACGTTGAGCGCGCCTGCAAA CAACAACAACAACAAGCGCCGCCGCGGCCACCGCGTCCTGCTCCTTCGAAGCGCCGC CGCCGCCGGTGGAGCCGCCTCCGCGCCCCTGGCCGTCCGGAGCGCCCGGCCGCTG GTGTATGTCGCGCCTGCCCGGGACGGGCTGAAGCCGGCGGCGGGGCCGGACCGCAG GCGCCGAGCAGGGCGAGGGCGGCCAGGGCAGCCGCCTGCCGCCAAGCCCCGAGCG CCGCTGCTCGAGGAAACGCTTTCGGCCGGGAGCTGCGGCCGCCGCCAGCAGTTTTCA TGTTTGGGATTCAGGAGAATATTCCGCGCGGGGGGACGACCATGAAGGAGGAGCCGC TGGGCAGCGGCATGAACCCGGTGCGCTCGTGGATGCACACGGCGGGCGTGGTGGAC GCCAACACGGCCGCCCAGAGCGGCGTGGGGCTGGCGCGGGCGCACTTCGAGAAGCA GCCGCCTTCCAACCTCCGGAAATCCAATTTCTTCCACTTCGTGCTGGCGCTCTACGATA GGCAGGGGCAGCCGGTGGAGATTGAAAGGACCGCTTTTGTGGACTTTGTGGAGAAAG AGAAAGAGCCAAACAACGAGAAAACCAACAACGGCATCCACTATAAACTCCAGTTATTG TACAGCAACGGAGTCAGAACAGAGCAAGATCTGTATGTTCGCCTCATAGATTCAATGAC CAAACAGGCCATCGTCTACGAGGGCCAGGACAAGAACCCGGAGATGTGCCGTGTGCT GCTGACCCACGAGATCATGTGCAGCCGGTGCTGTGACAAGAAAAGTTGTGGCAATAGA AACGAAACGCCCTCAGACCCTGTAATCATTGACAGATTCTTTCTAAAGTTTTTCCTCAAG TGCAATCAGAACTGTTTGAAGAATGCAGGCAACCCTCGAGATATGCGGAGATTCCAGG TTGTTGTATCGACAACAGTCAACGTGGACGGCCACGTGCTGGCCGTGTCAGACAACAT GTTTGTGCACAACAATTCCAAACACGGGAGGCGGGCCCGCCGCCTAGACCCGTCAGA AGCCACTCCGTGCATCAAGGCCATCAGTCCCAGTGAAGGCTGGACCACGGGGGGTGC CACCGTCATCATAATTGGCGACAACTTCTTTGACGGGCTGCAAGTTGTATTCGGAACTA TGTTGGTGTGGAGCGAGCTGATAACTCCCCATGCCATCCGAGTCCAGACCCCGCCGA GGCACATTCCTGGCGTCGTCGAAGTGACCCTCTCCTACAAATCCAAGCAGTTCTGCAA AGGTGCTCCTGGGCGCTTTGTCTACACCGCCCTTAATGAACCAACCATAGATTACGGC TTTCAGAGGTTGCAGAAAGTGATCCCAAGACATCCGGGTGATCCCGAAAGGTTACCCA AGGAGGTGTTACTGAAGCGGGCGGCGGACCTGGTGGAAGCCTTATACGGAATGCCTC ACAACAACCAGGAGATCATCTTGAAGCGAGCGGCGGACATCGCCGAGGCGCTGTACA GCGTTCCCCGCAATCACAACCAGATCCCCACCCTGGGCAACAACCCTGCACACACGG GCATGATGGGCGTCAACTCCTTCAGCAGCCAGCTAGCCGTCAACGTGTCAGAGACGTC ACAAGCCAACGACCAAGTCGGCTACAGTCGCAATACAAGCAGCGTGTCCCCGCGAGG CTACGTCCCCAGCAGTACTCCCCAGCAGTCCAATTACAACACAGTCAGCACTAGCATG AATGGATATGGAAGTGGCGCCATGGCCAGTCTAGGGGTCCCTGGCTCGCCTGGATTTC TTAATGGCTCCTCCGCTAACTCTCCCTACGGCATGAAACAGAAGAGCGCCTTCGCGCC CGTGGTCCGGCCCCAAGCCTCTCCTCCTCCTTCCTGCACCAGCGCCAACGGGAATGG ACTGCAAGCTATGTCTGGGCTGGTAGTCCCGCCAATGTGAGGGACTTCTGTTTACCTT CCGCAGCACCCAGCATCAAAGGACGGACTTCAGGGGACACGTTTAGTATATTAAGACA TGCTGATGGAAACAGTATCTTCAAAAAAATCAGCAGCAATTGAAATGCTACAAAAGACTT TGTTTAAAGATTTTATTTAAACTATTAAGAATCAACATGCAAACAGCCTACTTCTTCATGA ACAATTCCATTTTATTGACTGAACTTTTCTCATATTTTCACATTTCTCAGTCCTGAAGAAT AAGGAAAACAAAGCGACGCCTATTTTGTATAAAGTTTCCGACTCCGTCTTGGCCATGTC TAGTAATTGCTATGTGTTGGGAGAAACTTTGTGAATGCACCATTTTGATGATCATGAAAC GCTGATGAAAAATGCCTCCAAACATTTTTCTGTACTCATACTTAGATTCACAATGGTTGT GTATCTCTATAATGTGAAATATTTTTTTGTGGTGATAAAAAGAGGGCCAAGGAGGTATGA GCCATCAGACTGGAAAAAAGGATGACTATATGATGAGGAGAAACTGGGGTGGCAGGGA GGGAGGGAGGGTTTATCACTGCTTAACTTCATCTTCATGAAATGAAACTTTGTAACTTAT TGTAGTTAGAAATTGTAACTTTGATATTGAATTCTCTTGCCTTCAACAAGCACACTGACA GAGAAAAAATGCTACTGTCTGTTGGTTCCAATATTCTCCCACTGCTAGAGCTTCCTGTTA AGCAAGTGTGATCTGCAACATTTTTTCAACTTTTGCTAGCACTGTATACTATTGCATTCT TAGGCTACTGTGAGGTCTATGTTTCTTGTACCAGAAATTGTCCTTTTGACTTCTAGATCC TTCTTCCCTAATGTGTTTTGTATGTGGTTATAAAATTGTAGACTTTTGTGATTTTGCCAAA GTTGTAGCTAAATATTTATACACTTGTCTTGAATTTTTTTCAGATCCACTTAAAATATTTA GAAAAACAAGTTTTATTCCTTATGTGTCTTATAAGGAATAAAATGGTCTTCATTTGACACT TACTTTCCCATGAACACTTGCAGTTGCTAAGGGACTTTATTTTGTAACATATCAATTATAA ATATTGTATTTATCTTTGAAATTTTGTACATTGCTTTTCCCACCTTTTCCTTTTTCTTCTTT CTTGTCTGTATTGGTTTTTGATCACGGCCTGGTGTTGTGATACTGGGAAGAGCATTAGC CAAGAACTTGTCTCTTTGATCTGTTTCGTTAAGCTGAACCAGTGTCTTTACATTTCATTT GTACTTCAAAAAATATGCTATTGTTTAGACTTTCCATCCTTTTTTTTTTTATTTTGAGGAAA AGTCAAATTCATTGTTTATTTTTATATTATTTTTAAGTTATCTGAACAAATACTTTTGAAAA AAAAGTTTGTTGTATAGTCAAAACAAATCGGTGCCACCCGGCCGTGACAAATCCTAGTA GATTCTGTGCATGTGGAGCGGCCGCGAAGAGGTGACACCGTTTGGGGCTGTGTCCTT ATTTTATTTTATTTTTTTGTAGAATGTAAAAAGTCATTTTAGATGCCACCCATTGACTTTG CCACATAGCTGAACTGTGTTTACTGGAAAAATTCAGAGGCCTAAAGTTTAAAATAAAATT TACTTCTGATGTTTTAATTAAAATGTTTGCCACATTAACTTTTCTGATGCCTTAAAAGTGA ACTTCTTTAAAGAACCTTTGTGCTATTTTATCACAGGCTTACACTACAATTGTTAATAAAT ACTTCATTTGGAGATGTATGGTGTAAAACACACAAACACACACAAAAAAGCACAAGCCC GCTGCATGACCCGTCTCTCCTTTCTGGGACTATTTCTGCTGCGTCCTGCACCCTCCTG GGCCCACCTCCGATTCACAGAGGTTTCAGGGGGACCCAAATCACTGCTGGTTTTCAAT TTTTTTTTAACAATACATTTTTGTGGTCAGTTCCAACAGCACTGTCCGTACTTTTAAAACT GGAATGACCTCCTTCAGATATCGTGCCTCTTAGTGCCAAACCCACAGTGAGACCAAAA GTGTCAGGTGTTTTTTTTTTTTTCTTTCTCCTTTGCACTAAGTGCTTTGCAGACACGGCA CAGCAAACATTTTGCAAACTGCAGCAGAAATCGAATTTAAAACAAAAAGGAGGGACTTT AAAATACCTTCTTGACAAAAATCAACAATGCACAACTTACAAAGTGTTCATTCTAGGGAC AAAATTAAATAAACAGAATGTCCCCAGGAGTCAGCAGGTCACAGTCTGGCTTTGTGATG GTTGACAAGGTCTAGCTACATGGGAAAGCCTGAGAAGTCACTTTGGAACTAAATTGCCT CCATTTTATTTTGTACGAGTAAGGGTTTGATCTACAAAAGAGCTCACATGGACGCACTG AGAACGCCTGCCAGCTTCCCCATGCCCTCACTTGGTTTGTGTTTTAGGTTAAGTAGTCA ATGCCCACATCACTTCACTGTCTCAAGACTGAGCACTTCACTAAATGGTAGATTTTACTG TTAAAGACCCTACAATAAGATTGTTTTATCTGTACATTTTTTCAGATATTTAACTGTATAA AAATGTTCATTTTACACAATATTTAATTAAAGTATTTCTTGTCTGTGAATTTCACTTTTGGT AATTTTCTCTGTTTTTGATTATTAAAATGACTAAACACTAA. (SEQ ID NO:226; NM_001005463.3), or a nucleic acid sequence that hybridizes to a nucleic acid sequence consisting of SEQ ID NO:226 under stringent hybridization conditions.

In some embodiments, MAF bZIP transcription factor (MAF, CCA4, AYGRP, c-MAF, CTRCT21) comprises the amino acid sequence: MASELAMSNSDLPTSPLAMEYVNDFDLMKFEVKKEPVETDRIISQCGRLIAGGSLSSTPMS TPCSSVPPSPSFSAPSPGSGSEQKAHLEDYYWMTGYPQQLNPEALGFSPEDAVEALISNS HQLQGGFDGYARGAQQLAAAAGAGAGASLGGSGEEMGPAAAVVSAVIAAAAAQSGAGP HYHHHHHHAAGHHHHPTAGAPGAAGSAAASAGGAGGAGGGGPASAGGGGGGGGGGG GGGAAGAGGALHPHHAAGGLHFDDRFSDEQLVTMSVRELNRQLRGVSKEEVIRLKQKRR TLKNRGYAQSCRFKRVQQRHVLESEKNQLLQQVDHLKQEISRLVRERDAYKEKYEKLVSS GFRENGSSSDNPSSPEFFM. (SEQ ID NO:227; NP_001026974.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:227)

In some embodiments, the nucleic acid sequence encoding MAF comprises the nucleic acid sequence: CTTGCTGGATCAGAGGCTTTAAAATCTTTTTTCATCTTCTAGCTGTAGCTCGGGCTGCTT GTCGGCTTGGCCTCCCCCTCCCCCCTTTGCTCTCTGCCTCGTCTTTCCCCAGGACTTC GCTATTTTGCTTTTTTAAAAAAAGGCAAGAAAGAACTAAACTCCCCCCTCCCTCTCCTCC AGTCGGGCTGCACCTCTGCCTTGCACTTTGCACAGAGGTAGAGAGCGCGCGAGGGAG AGAGAGGAAAGAAAAAAAATAATAAAGAGAGCCAAGCAGAAGAGGAGGCGAGAAGCAT GAAGTGTTAACTCCCCCGTGCCAAGGCCCGCGCCGCCCGGACAGACGCCCGCCGCG CCTCCAGCCCCGAGCGGACGCCGCGCGCGCCCTGCCTGCAGCCCGGGCCGGCGAG GCGAGCCCTTCCTTATGCAAAGCGCGCAGCGGAGCGGCGAGCGGGGGACGCCGCGC ACCGGGCCGGGCTCCTCCAGCTTCGCCGCCGCAGCCACCACCGCCGCCACCGCAGC TCGCGGAGGATCTTCCCGAGCCTGAAGCCGCCGGCTCGGCGCGCAAGGAGGCGAGC GAGCAAGGAGGGGCCGGGGCGAGCGAGGGAGCACATTGGCGTGAGCAGGGGGGAG GGAGGGCGGGCGCGGGGGGCGCGGGCAGGGCGGGGGGGTGTGTGTGTGAGCGCG CTCGGAGGTTTCGGGCCAGCCACCGCCGCGCAAGCTAGAAGCGCCCCAGCCCGGCA AGCTGGCTCACCCGCTGGCCACCCAGCACAGCCCGCTGGCCCCTCTCCTGCAGCCCA TCTGGCGGAGCGGCGGCGGCGGCGGCGGCGGCGGCAGGAGAATGGCATCAGAACT GGCAATGAGCAACTCCGACCTGCCCACCAGTCCCCTGGCCATGGAATATGTTAATGAC TTCGATCTGATGAAGTTTGAAGTGAAAAAGGAACCGGTGGAGACCGACCGCATCATCA GCCAGTGCGGCCGTCTCATCGCCGGGGGCTCGCTGTCCTCCACCCCCATGAGCACGC CGTGCAGCTCGGTGCCCCCTTCCCCCAGCTTCTCGGCGCCCAGCCCGGGCTCGGGCA GCGAGCAGAAGGCGCACCTGGAAGACTACTACTGGATGACCGGCTACCCGCAGCAGC TGAACCCCGAGGCGCTGGGCTTCAGCCCCGAGGACGCGGTCGAGGCGCTCATCAGC AACAGCCACCAGCTCCAGGGCGGCTTCGATGGCTACGCGCGCGGGGCGCAGCAGCT GGCCGCGGCGGCCGGGGCCGGTGCCGGCGCCTCCTTGGGCGGCAGCGGCGAGGAG ATGGGCCCCGCCGCCGCCGTGGTGTCCGCCGTGATCGCCGCGGCCGCCGCGCAGAG CGGCGCGGGCCCGCACTACCACCACCACCACCACCACGCCGCCGGCCACCACCACC ACCCGACGGCCGGCGCGCCCGGCGCCGCGGGCAGCGCGGCCGCCTCGGCCGGTGG CGCTGGGGGCGCGGGCGGCGGTGGCCCGGCCAGCGCTGGGGGCGGCGGCGGCGG CGGCGGCGGCGGAGGCGGCGGGGGCGCGGCGGGGGCGGGGGGCGCCCTGCACCC GCACCACGCCGCCGGCGGCCTGCACTTCGACGACCGCTTCTCCGACGAGCAGCTGGT GACCATGTCTGTGCGCGAGCTGAACCGGCAGCTGCGCGGGGTCAGCAAGGAGGAGG TGATCCGGCTGAAGCAGAAGAGGCGGACCCTGAAAAACCGCGGCTATGCCCAGTCCT GCCGCTTCAAGAGGGTGCAGCAGAGACACGTCCTGGAGTCGGAGAAGAACCAGCTGC TGCAGCAAGTCGACCACCTCAAGCAGGAGATCTCCAGGCTGGTGCGCGAGAGGGACG CGTACAAGGAGAAATACGAGAAGTTGGTGAGCAGCGGCTTCCGAGAAAACGGCTCGA GCAGCGACAACCCGTCCTCTCCCGAGTTTTTCATGTGAGTCTGACACGCGATTCCAGC TAGCCACCCTGATAAGTGCTCCGCGGGGGTCCGGCTCGGGTGTGGGCTTGCTAGTTC TAGAGCCATGCTCGCCACCACCTCACCACCCCCACCCCCACCGAGTTTGGCCCCCTTG GCCCCCTACACACACACAAACCCGCACGCACACACCACACACACACACACACACACAC ACACACCCCACACCCTGCTCGAGTTTGTGGTGGTGGTGGCTGTTTTAAACTGGGGAGG GAATGGGTGTCTGGCTCATGGATTGCCAATCTGAAATTCTCCATAACTTGCTAGCTTGT TTTTTTTTTTTTTTTACACCCCCCCGCCCCACCCCCGGACTTGCACAATGTTCAATGATC TCAGCAGAGTTCTTCATGTGAAACGTTGATCACCTTTGAAGCCTGCATCATTCACATATT TTTTCTTCTTCTTCCCCTTCAGTTCATGAACTGGTGTTCATTTTCTGTGTGTGTGTGTGTT TTATTTTGTTTGGATTTTTTTTTTTAATTTTACTTTTAGAGCTTGCTGTGTTGCCCACCTTT TTTCCAACCTCCACCCTCACTCCTTCTCAACCCATCTCTTCCGAGATGAAAGAAAAAAAA AAGCAAAGTTTTTTTTTCTTCTCCTGAGTTCTTCATGTGAGATTGAGCTTGCAAAGGAAA AAAAAATGTGAAATGTTATAGACTTGCAGCGTGCCGAGTTCCATCGGGTTTTTTTTTTAG CATTGTTATGCTAAAATAGAGAAAAAAATCCTCATGAACCTTCCACAATCAAGCCTGCAT CAACCTTCTGGGTGTGACTTGTGAGTTTTGGCCTTGTGATGCCAAATCTGAGAGTTTAG TCTGCCATTAAAAAAACTCATTCTCATCTCATGCATTATTATGCTTGCTACTTTGTCTTAG CAACAATGAACTATAACTGTTTCAAAGACTTTATGGAAAAGAGACATTATATTAATAAAAA AAAAAAGCCTGCATGCTGGACATGTATGGTATAATTATTTTTTCCTTTTTTTTTCCTTTTG GCTTGGAAATGGACGTTCGAAGACTTATAGCATGGCATTCATACTTTTGTTTTATTGCCT CATGACTTTTTTGAGTTTAGAACAAAACAGTGCAACCGTAGAGCCTTCTTCCCATGAAAT TTTGCATCTGCTCCAAAACTGCTTTGAGTTACTCAGAACTTCAACCTCCCAATGCACTGA AGGCATTCCTTGTCAAAGATACCAGAATGGGTTACACATTTAACCTGGCAAACATTGAA GAACTCTTAATGTTTTCTTTTTAATAAGAATGACGCCCCACTTTGGGGACTAAAATTGTG CTATTGCCGAGAAGCAGTCTAAAATTTATTTTTTAAAAAGAGAAACTGCCCCATTATTTTT GGTTTGTTTTATTTTTATTTTATATTTTTTGGCTTTTGGTCATTGTCAAATGTGGAATGCT CTGGGTTTCTAGTATATAATTTAATTCTAGTTTTTATAATCTGTTAGCCCAGTTAAAATGT ATGCTACAGATAAAGGAATGTTATAGATAAATTTGAAAGAGTTAGGTCTGTTTAGCTGTA GATTTTTTAAACGATTGATGCACTAAATTGTTTACTATTGTGATGTTAAGGGGGGTAGAG TTTGCAAGGGGACTGTTTAAAAAAAGTAGCTTATACAGCATGTGCTTGCAACTTAAATAT AAGTTGGGTATGTGTAGTCTTTGCTATACCACTGACTGTATTGAAAACCAAAGTATTAAG AGGGGAAACGCCCCTGTTTATATCTGTAGGGGTATTTTACATTCAAAAATGTATGTTTTT TTTTCTTTTCAAAATTAAAGTATTTGGGACTGAATTGCACTAAGATATAACCTGCAAGCA TATAATACAAAAAAAAATTGCAAAACTGTTTAGAACGCTAATAAAATTTATGCAGTTATAA AAATGGCATTACTGCACAGTTTTAAGATGATGCAGATTTTTTTACAGTTGTATTGTGGTG CAGAACTGGATTTTCTGTAACTTAAAAAAAAATCCACAGTTTTAAAGGCAATAATCAGTA AATGTTATTTTCAGGGACTGACATCCTGTCTTTAAAAAGAAATGAAAAGTAAATCTTACC ACAATAAATATAAAAAAATCTTGTCAGTTACTTTTCTTTTACATATTTTGCTGTGCAAAATT GTTTTATATCTTGAGTTACTAACTAACCACGCGTGTTGTTCCTATGTGCTTTTCTTTCATT TTCAATTCTGGTTATATCAAGAAAAGAATAATCTACAATAATAAACGGCATTTTTTTTTGA TTCTGTACTCAGTTTCTTAGTGTACAGTTTAACTGGGCCCAACAACCTCGTTAAAAGTGT AAAATGCATCCTTTTCTCCAGTGGAAGGATTCCTGGAGGAATAGGGAGACAGTAATTCA GGGTGAAATTATAGGCTGTTTTTTGAAGTGAGGAGGCTGGCCCCATATACTGATTAGCA ATATTTAATATAGATGTAAATTATGACCTCATTTTTTTCTCCCCAAAGTTTTCAGTTTTCAA ATGAGTTGAGCCATAATTGCCCTTGGTAGGAAAAACAAAACAAAACAGTGGAACTAGGC TTCCTGAGCATGGCCCTACACTTCTGATCAGGAGCAAAGCCATCCATAGACAGAGGAG CCGGACAAATATGGCGCATCAGAGGTGGCTTGCGCACATATGCATTGAACGGTAAAGA GAAACAGCGCTTGCCTTTTCACTAAAGTTGACTATTTTTCCTTCTTCTCTTACACACCGA GATTTTCTTGTTAGCAAGGCCTGACAAGATTTAACATAAACATGACAAATCATAGTTGTT TGTTTTGTTTTGCTTTTCTCTTTAACACTGAAGATCATTTGTCTTAAATAGGAAAAAGAAA ATCCACTCCTTACTTCCATATTTCCAAGTACATATCTGGTTTAAACTATGTTATCAAATCA TATTTCACCGTGAATATTCAGTGGAGAACTTCTCTACCTGGATGAGCTAGTAATGATTTC AGATCATGCTATCCCCAGAAATAAAAGCAAAAAATAATACCTGTGTGGAATATAGGCTG TGCTTTGATTTACTGGTATTTACCCCAAAATAGGCTGTGTATGGGGGCTGACTTAAAGA TCCCTTGGAAAGACTCAAAACTACCTTCACTAGTAGGACTCCTAAGCGCTGACCTATTT TTAAATGACACAAATTCATGAAACTAATGTTACAAATTCATGCAGTTTGCACTCTTAGTC ATCTTCCCCTAGCACACCAATAGAATGTTAGACAAAGCCAGCACTGTTTTGAAAATACA GCCAAACACGATGACTTTTGTTTTGTTTTCTGCCGTTCTTAAAAGAAAAAAAGATAATAT TGCAACTCTGACTGAAAGACTTATTTTTAAGAAAACAGGTTGTGTTTGGTGCTGCTAAGT TCTGGCCAGTTTATCATCTGGCCTTCCTGCCTATTTTTTACAAAACACGAAGACAGTGT GTAACCTCGACATTTTGACCTTCCTTTATGTGCTAGTTTAGACAGGCTCCTGAATCCACA CTTAATTTTGCTTAACAAAAGTCTTAATAGTAAACCTCCCCTCATGAGCTTGAAGTCAAG TGTTCTTGACTTCAGATATTTCTTTCCTTTTTTTTTTTTTTTCCTCATCACAACTAAGAGAT ACACAAACTCTGAAGAAGCAGAAATGGAGAGAATGCTTTTAACAAAAAAGCATCTGATG AAAGATTTTAGGCAAACATTCTCAAAATAAGAGTGATATTCTGGATGTAGTTATTGCAGT TATCTCATGACAAATGAGGCCTGGATTGGAAGGAAAATATAGTTGTGTAGAATTAAGCA TTTTGATAGGAATCTACAAGGTAGTTGAATATAATAAGCAGGTTTGGGCCCCCAAACTTT AGAAAATCAAATGCAAAGGTGCTGGCAAAAATGAGGTTTGAGTGGCTGGCTGTAAGAG AAGGTTAACTCCTAGTAAAAGGCATTTTTAGAAATAACAATTACTGAAAACTTTGAAGTA TAGTGGGAGTAGCAAACAAATACATGTTTTTTTTTTCTTACAAAGAACTCCTAAATCCTG AGTAAGTGCCATTCATTACAATAAGTCTCTAAATTTAAAAAAAAAAAAATCATATGAGGA AATCTAGCTTTCCCCTTTACGCTGCGTTTGATCTTTGTCTAAATAGTGTTAAAATTCCTTT CATTCCAATTACAGAACTGAGCCCACTCGCAAGTTGGAGCCATCAGTGGGATACGCCA CATTTTGGAAGCCCCAGCATCGTGTACTTACCAGTGTGTTCACAAAATGAAATTTGTGT GAGAGCTGTACATTAAAAAAAATCATCATTATTATTATTATTTGCAGTCATGGAGAACCA CCTACCCCTGACTTCTGTTTAGTCTCCTTTTTAAATAAAAATTACTGTGTTAGAGAAGAA GGCTATTAAATGTAGTAGTTAACTATGCCTCTTGTCTGGGGGTTTCATAGAGACCGGTA GGAAAGCGCACTCCTGCTTTTCGATTTATGGTGTGTGCAAGTAAACAGGTGCATTGCTT TCAACCTGCCATACTAGTTTTAAAAATTCACTGAAATTACAAAGATACATATATATGCATA TATATAATGGAAAGTTTCCCGGAATGCAACAATTAGCATTTTAAAATCATATATAGGCAT GCACATTCTAAATAGTACTTTTTCATGCTTCATTGTTTCTCTGGCAGATAATTTTACTAAG AAGAAAAATAGATATTCGACTCCCCTTCCCTAAACAAATCCACGGGCAGAGGCTCCAGC GGAGCCGAGCCCCCTGGTTTTCTCGTAGGCCCTAGACGGTGTTGCATTTATCAGTGAT GTCAAACGTGCTCATTTGTCAGACATAGCTGTAAATGAAAACAATGTGTGGCAAAATAC AAAGTTAGTTAAATACA. (SEQ ID NO:228; NM 001031804.3), or a nucleic acid sequence that hybridizes to a nucleic acid sequence consisting of SEQ ID NO:228 under stringent hybridization conditions.

In some embodiments, nuclear protein 1 (NUPR1, P8, COM1) comprises the amino acid sequence: MATFPPATSAPQQPPGPEDEDSSLDESDLYSLAHSYLGPLIMPMPTSPLTPALVTGGGGRK GRTKREAAANTNRPSPGGHERKLVTKLQNSERKKRGARR. (SEQ ID NO:229; NP_001035948.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:229)

In some embodiments, the nucleic acid sequence encoding NUPR1 comprises the nucleic acid sequence: GCCAGGAAAGCAGAGACAGACAAAGCGTTAGGAGAAGAAGAGAGGCAGGGAAGACAA GCCAGGCACGATGGCCACCTTCCCACCAGCAACCAGCGCCCCCCAGCAGCCCCCAGG CCCGGAGGACGAGGACTCCAGCCTGGATGAATCTGACCTCTATAGCCTGGCCCATTCC TACCTCGGGCCTCTCATCATGCCTATGCCCACTTCACCTCTGACTCCTGCCTTGGTTAC AGGAGGTGGAGGCCGGAAAGGTCGCACCAAGAGAGAAGCTGCTGCCAACACCAACCG CCCCAGCCCTGGCGGGCACGAGAGGAAACTGGTGACCAAGCTGCAGAATTCAGAGAG GAAGAAGCGAGGGGCACGGCGCTGAGACAGAGCTGGAGATGAGGCCAGACCATGGA CACTACACCCAGCAATAGAGACGGGACTGCGGAGGAAGGAGGACCCAGGACAGGATC CAGGCCGGCTTGCCACACCCCCCACCCCTAGGACTTATTCCCGCTGACTGAGTCTCTG AGGGGCTACCAGGAAAGCGCCTCCAACCCTAGCAAAAGTGCAAGATGGGGAGTGAGA GGCTGGGAATGGAGGGGCAGAGCCAGGAAGATCCCCCAGAAAAGAAAGCTACAGAAG AAACTGGGGCTCCTCCAGGGTGGCAGCAACAATAAATAGACACGCACGGCAGCCACA GCTTGGGTGTGTGTTCATCCTTGTTCTTTGTGTGTTTTTGTTCGGGCATGTGTGTGCTT GCCTGTGCCTGCACATTCATGAGCCTGAGAGAGCATCTTTGATGTGTATTTGTGTTTGG TGTATGTATCTGGGGCAGGGAGTGTTCCTGCTCTTGCAGGGTCTACGCTGTGAATGCA GCTTTTGGTTTGTTTGCTTTTGCTTGCATATATTTTAGTGCATACATTTCTGTGGGCTCC TACGTAGTGGAAAGGAATTTCTTCTGCTTTTTTGCGATACTGCCCATGAAACACGGCCC TCCCCAGCACCTGTTTTTGTTGATTGTGTCCTGTTCATAGACGGGAACGCTACTTATGA GTGCCATCTAAAAGTCAGAGAAAACTGAGATTTAAAATATTAAAAGCCAGGGCCGGGG GCAGTGGCTCACGCCTGTAATCCCAGCACTTTGGGAGGCCGAGGTGGGTGGATCACA AAGTCAGGAGTTCAAGACCAGCCTGACCAACATGGTGAAACCCCATCTCCACTAAAAAT ACCAAAAATTAGCCAAGCATGGTGGCAGGTGCCTGTAATCCCAGCTGCTCAGGAGGCT GAGGCAGTAGAATCGCTTGAACCCAGGAGGTAGAGGTTGCAGTGAGCCGACATCGTG CCATTGCACTCCAGCCTGGGTGACAGAGGGAGACTCTGTCTCAAAACAAACAAACAAA CAAAAACTAAAGTCTGGGAGCAGTGGCTCATGCCTGTAATCACAGCAGTTTAGGAGGC CGAAGTGGGAGGATTACTTGAGCCTAGGAGTTTGAGACCAGCCTGAGCATCATAGTAA GACCCCATCTCTACAATTTTTTTTTTGAGACAGAGTCTCACTCTGTTGCTCAGGTTAGAG TGCAGTGGCACCATCTTGGCTCACTGCAACCTCTGCCTCCCGGGTTCAAGCAATTCTC GTGCCTACGCCCCCTGAGTAGCTGGGATTACAGGTGAGCACCACCACGCCTGGCTAAT TTTTGTTTTTTTGTTTTTTTGATACAGAGTCTCACTCTGTTGCTCAGGCTGGAGTGCAGT GGCATGATCGCAGCTCACTGCAACCTCCGCCTCCTGGGTTCAAGCTATTCTCCTGCTT CAGCCTCCTGAGTAGCTGGGACTACAGGCACCTGCCACCATGCCTGGCTAATTTTTGT ATTTTTAGTAGAAACAGAGTTTCACCATGTTGGCCAGGATGGTCTCAATCTCTTGACCT CATGATCCACCCACCTTGGCCTCCCAAAGTGCTGGGATTACAGGCGTCAGCCACCACG CCTGGCCAATTTTTGTATTTTTAGTAGAGATGGGGTTTCATCATGTTGGCCAGGCTGGT CTCAAACTCCTGGCCTCAAATGATCTGCCCACCTCAGCCTTCCAGCCTTTGGGAGGCC AAGGAGGGAGGATCGCTTGAGGCCAGGAGTTCGAGACCAGCCTAGGCAACATACCAA GGCCCTGTCTCTACAAAAATTTAAAAATTAGCAAAGCATGGTGGCTCATGCCTGTAGTC CTAGTTGCTCAGAGGCTGAAGTTGGAGGATCCCTTGAACCCAGTTGGAGGCTACAGTA AGCCATGATGGTGCCACTGCACTGCAGCCTGAGCAATAGAGTGAAACCATGTATTGAA AAAGAAAGAAAGAAAGAAAGAGAAAAAGAAGGAAGGAAGGAAAAGAAAGGAAGGAAGA AAGAAAAGAAAGAGAGAAAGGAGAGAAAAAGAAGAAAGAGAGAGAAAGAGAAGAAAGA AAGAAAGAAAGAAAGAAAGAAAGAAAGAAAGAAAGAAAGAAAGGAAGGAAGGAAAGAA AGAAAGAAAGAAGGAAAGAAAGAAAGAAAGAAAGAAAGAAAGAAAGAAAGAAAAGAGC GAGCCCCGGCACAGTGGCTCACACCTGTAATCCCAGCACTTTGGGAGGCCAAGGCAG GCGGATCACCTGAGGTCAGTAGTTCGAGTTCGAGGCAGAAGTTGTGGTGAGCCAAGAT CACGCCATTGCACTCCAGCCTGGGCAACAAGAGCAAAACTCCATCTCAAAAAAAGAAA AAGAGAGAGAAAGAAAGAGAGAAGGAGGGAAAGAAAGAGGAAGGAAAGGAGGGAGG GAAGGAGGGAAGGAGGAAAGGAGGGAAGGAAATATTAAGGGATTTGCCCTAGCTCAC TCAGGGCTCATATTCAGGGATCTTATTGTCTTCAGATATGAGGTGGGTCCCCAGGCCAA GGGGGCTGTGGGACAGGGTTCTGGGAAGTGAGATGGGGGAGGGAGATTCTTATTCAG TGCTATGTCTCAGGACCAGAGTTGAAGAAAAATATGGGGCAAACAGGAAGTGTGGTTG GAGCTGAGATTATTTGTTGATTGAGATACAGGTTTGGGTTGCTATTATGAAGAATCAAAA CAGGATACAAAGTTACTCCTCTCTTACTTAGCCCCAAGTCCTCCTGGGTTCAAATGGCA GCTCCATTGTGTTGAGGATTCCAGCTATTTTTACTCACTGTTCTGCCACACCCAAAACAT GGCTTCCACTTTGAGGCCCAAAATAGCTACTCCAATACCTGCCATCACGTTTGCATCTC AGCCTGTGGAAAGAGTGTGGAAGAGACAGAGGAGGCCACACCTCTTCAATTTTAGGGC ATAATCTAAAAGTAGAACACATCACATCTGCTCCGAATGTGGCTACATGGCCACACCCA ACTACGAGAGAGGTTGGGAAATGTAGCATTGAGCTGCTTAGCCACGTGGTAGCCAAAA CTTGGGCAACAGCAGGGTTTTATGATGAAAGGGAACCAGAGAGAGTGAATACACGAAG AAGCTAGCAAGAAACATTGCCCACATGCAGACTCAACAGATGTCGGGGGAGAAAACTC TGAGATTGGGCCCCGGGCAGAGCCTGTTTCCTTTTCCCCTGTCCCTTTCACATAAGCC CCACACTGCAGAACTTGGAGTCAGTGGTTCATGGGGACCCAAGGACATCCACAGAAGA CCGAAGTACCTGATAGAGCAGGAACAGATGCACGTCAGACTACGACCAGCTTGAACCC TTGGGGACCTGTATCCATCCCCTTCCCTTGAGTCCAGCCTAATCACAGCCACAGAATGA ATCCATGACCCGGAAGGACATTTAAGGACCTCTGGCCCAACTCTCCCCACGCAACAGT GGGATTGCCTCTGCTTGTTTGAATACCACTATGGAGGGGGAACTTGCTACATTTAAAGG TTGCTGAATTTTTTTGTTTTGTTTTGGGAGATGGAGTCTCTCTCTGTCGCCCAGGCTGG AGTGCAGTGGTGTGATCTCGACCCACTGCAACCTCCGCTTCCTGGGTTCAAGCGATTT TCATGCCTCAGCCTCCCTAGTAGCTGGGACTACAGGTGTGGGCCACCACACCTGGCTA ATTTTTGTATTTTTAGTAGAGACGGGGTTTCACCATGTTGCCCAGGCTGGTCTCAAACT CCCGACCTCAGGTGATCTGCCTGCCTTGGCCTCCCAAAGTGTTGGGATTACAGGCTTG AGCCACACCACCCGGCAAAGGTTGCCTAATTCTATCTCAGGGCAAGTCTGTTCATGTG GGCTGAAATCTGTCTCTCATACCTGCCCTTAAATAAGCTTCTGTTTTTCTGGTCCTTTCT GTCTGAGATGACTTTAATTCCTCCCCCGACCCCATCTCATGAGGCTCAGCTTAGATGTT TCCTTCTCTTCCTCCCTGATGGCTGCGTGCAGAAGCTCATACCTGCAATCCCAGCACTT TGGGAGGCCGAGGTGGGAGGATTGCTTGGGCCCAGGAGTTCTAGGCCAGCCTAGCAA CACAGCAAGACCCTCTCTCTACAAAAAATTAAAAATTAGCCAGGTGTGGTGGCTTGTGC CTGTGGGCCCACCTACTCAGGAGGCTGAGGTGAGAGGATCTCTTGAGTCTGGGAGGT GGAGGCTGCAGTGAGCCATGATCTCACTCCAGCCTGGGCGTCAGAGTGAGACCTGTC TCAAAAAAACAAAAACCAAAGTTTTCCTTAACTTGCAATCCTCCACCCATCTCCTGACCT CCACCCCAGGCCGACCCTCCTCTGGGATCACACGGAAGCCTGGGCCCAGAATCTTAC AGTCTACCTTGGAAATCCTTCAGATTTTGCCTCCAACTCCTCTTTCCATACAAGTTACAT ATTAACTCAATCAATGCTTATTGGATGCCTTCTGAGTTACCAGGATGCTGCTGGAGGCC TGTGCTGCTGAGAGGGACAAATAGAATCCTGATATGACTGGAACCAGGAATAGGGCCA CCATCAGCTCTAGAACCCAGGGAGCAAATGGCAGGAGCAACACCAGGCAGTCGGCCC TCAGCCCAGCACAGACCTGGTGTGTTGGCTTCAGAAAATCCTGGAAACATCTCTGCTG TGTTTGGAGCTCCTATTCTGTGAGAAGTGTTTTATAGGCTTTGCTCCATTTAATCCTTAC GATAACCCAATAGGACATACTATTAATGATCCCCTTTTTTTCAGACCGGAAAACTGAGGT TCGGAGACATTAAGTAATTTGCCTAAAGTCACACAGCCACATCCTGTAGGACTTGGAAT TATGAACTCAGGTCTATCTGAATTTTAAAGCCTGGATTTTTCCCCCTTTGCTACATGCCT GGGAAGAACCATGTATTGACAATGAAGAATCCTAAACTCTCGTTAA. (SEQ ID NO:230; NM_001042483.2), or a nucleic acid sequence that hybridizes to a nucleic acid sequence consisting of SEQ ID NO:230 under stringent hybridization conditions.

In some embodiments, twist family bHLH transcription factor 1 (TWIST1, CRS, CSP, SC3, ACS3, CRS1, BPES2/3, SWCOS, TWIST) comprises the amino acid sequence: MMQDVSSSPVSPADDSLSNSEEEPDRQQPPSGKRGGRKRRSSRRSAGGGAGPGGAAG GGVGGGDEPGSPAQGKRGKKSAGCGGGGGAGGGGGSSSGGGSPQSYEELQTQRVMA NVRERQRTQSLNEAFAALRKIIPTLPSDKLSKIQTLKLAARYIDFLYQVLQSDELDSKMASCS YVAHERLSYAFSVWRMEGAWSMSASH. (SEQ ID NO:231; NP_000465.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:231)

In some embodiments, the nucleic acid sequence encoding TWIST1 comprises the nucleic acid sequence: AACTCCCAGACACCTCGCGGGCTCTGCAGCACCGGCACCGTTTCCAGGAGGCCTGGC GGGGTGTGCGTCCAGCCGTTGGGCGCTTTCTTTTTGGACCTCGGGGCCATCCACACC GTCCCCTCCCCCTCCCGCCTCCCTCCCCGCCTCCCCCGCGCGCCCTCCCCGCGGAG GTCCCTCCCGTCCGTCCTCCTGCTCTCTCCTCCGCGGGCCGCATCGCCCGGGCCGGC GCCGCGCGCGGGGGAAGCTGGCGGGCTGAGGCGCCCCGCTCTTCTCCTCTGCCCCG GGCCCGCGAGGCCACGCGTCGCCGCTCGAGAGATGATGCAGGACGTGTCCAGCTCG CCAGTCTCGCCGGCCGACGACAGCCTGAGCAACAGCGAGGAAGAGCCAGACCGGCA GCAGCCGCCGAGCGGCAAGCGCGGGGGACGCAAGCGGCGCAGCAGCAGGCGCAGC GCGGGCGGCGGCGCGGGGCCCGGCGGAGCCGCGGGTGGGGGCGTCGGAGGCGGC GACGAGCCGGGCAGCCCGGCCCAGGGCAAGCGCGGCAAGAAGTCTGCGGGCTGTGG GGCGGCGGCGGCGCGGGCGGCGGCGGCGGCAGCAGCAGCGGCGGCGGGAGTCCG CAGTCTTACGAGGAGCTGCAGACGCAGCGGGTCATGGCCAACGTGCGGGAGCGCCA GCGCACCCAGTCGCTGAACGAGGCGTTCGCCGCGCTGCGGAAGATCATCCCCACGCT GCCCTCGGACAAGCTGAGCAAGATTCAGACCCTCAAGCTGGCGGCCAGGTACATCGA CTTCCTCTACCAGGTCCTCCAGAGCGACGAGCTGGACTCCAAGATGGCAAGCTGCAGC TATGTGGCTCACGAGCGGCTCAGCTACGCCTTCTCGGTCTGGAGGATGGAGGGGGCC TGGTCCATGTCCGCGTCCCACTAGCAGGCGGAGCCCCCCACCCCCTCAGCAGGGCCG GAGACCTAGATGTCATTGTTTCCAGAGAAGGAGAAAATGGACAGTCTAGAGACTCTGG AGCTGGATAACTAAAAATAAAAATATATGCCAAAGATTTTCTTGGAAATTAGAAGAGCAA AATCCAAATTCAAAGAAACAGGGCGTGGGGCGCACTTTTAAAAGAGAAAGCGAGACAG GCCCGTGGACAGTGATTCCCAGACGGGCAGCGGCACCATCCTCACACCTCTGCATTCT GATAGAAGTCTGAACAGTTGTTTGTGTTTTTTTTTTTTTTTTTTTTGACGAAGAATGTTTTT ATTTTTATTTTTTTCATGCATGCATTCTCAAGAGGTCGTGCCAATCAGCCACTGAAAGGA AAGGCATCACTATGGACTTTCTCTATTTTAAAATGGTAACAATCAGAGGAACTATAAGAA CACCTTTAGAAATAAAAATACTGGGATCAAACTGGCCTGCAAAACCATAGTCAGTTAATT CTTTTTTTCATCCTTCCTCTGAGGGGAAAAACAAAAAAAAACTTAAAATACAAAAAACAA CATTCTATTTATTTATTGAGGACCCATGGTAAAATGCAAATAGATCCGGTGTCTAAATGC ATTCATATTTTTATGATTGTTTTGTAAATATCTTTGTATATTTTTCTGCAATAAATAAATAT AAAAAATTTAGAGAA. (SEQ ID NO:232; NM_0004744), or a nucleic acid sequence that hybridizes to a nucleic acid sequence consisting of SEQ ID NO:232 under stringent hybridization conditions

In some embodiments, twist family bHLH transcription factor 2 (TWIST2, AMS, FFDD3, BBRSAY, DERMO1, SETLSS) comprises the amino acid sequence: MEEGSSSPVSPVDSLGTSEEELERQPKRFGRKRRYSKKSSEDGSPTPGKRGKKGSPSAQ SFEELQSQRILANVRERQRTQSLNEAFAALRKIIPTLPSDKLSKIQTLKLAARYIDFLYQVLQS DEMDNKMTSCSYVAHERLSYAFSVWRMEGAWSMSASH. (SEQ ID NO:233; NP_001258822.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:233)

In some embodiments, the nucleic acid sequence encoding Fos comprises the nucleic acid sequence: GCACAAGCCGGCCTTGAAATCAGAGCCTTTCCAGCAACTCCGAGAGCGTGTGCTCGG CGACCGCGGGCTTGGCCAGCGGCGCGCGCTCGGCGCCCCGGCGCCCCCAGCCCCA CGCGCGCCGGGCGGGCGCCATGGAGGAGGGCTCCAGCTCGCCCGTGTCCCCCGTGG ACAGCCTGGGCACCAGCGAGGAGGAGCTCGAGAGGCAGCCCAAGCGCTTCGGCCGG AAGCGGCGCTACAGCAAGAAGTCGAGCGAAGATGGCAGCCCGACCCCGGGCAAGCG CGGCAAGAAGGGCAGCCCCAGCGCGCAGTCCTTCGAGGAGCTGCAGAGCCAGCGCA TCCTGGCCAACGTGCGCGAGCGCCAGCGCACCCAGTCGCTCAACGAGGCCTTCGCGG CGCTGCGCAAGATCATCCCCACGCTGCCCTCTGACAAGCTGAGCAAGATCCAGACGCT CAAGCTGGCCGCCAGGTACATAGACTTCCTCTACCAGGTCCTGCAGAGCGACGAGATG GACAATAAGATGACCAGCTGCAGCTACGTGGCCCACGAGCGCCTCAGCTACGCCTTCT CCGTGTGGCGCATGGAGGGCGCGTGGTCCATGTCCGCCTCCCACTAGCGCCGCGCC ACCCACCTCCGGACCGGCGCGCCAGGGCTGTCCGTCGCGTCGGCGGCGCAAGTGGA ATTGGGATGCATTCGAGTCTGTAACTTCTGAAACCTGAACAACCTCAGGAGGCCCCCA CCTCTGCCCTCCACCAGCGTCGAGAGAAGGGACAGCAGTGACATCGGACAGAAGACC CGGGCTCCCGTCCTCCCCCAGGACGGTCCCCACATAGGAAGGGCACTCCCAGCCCTC TTGCTGGTGACATTGTCATGGTCATCTTGTTTCTGTTTGGATTTTTCTTCTGGGTCTTAT GTTTGGGGGGAGGTTTATTCTTTCTGAAAATGTCTAGATTCAGGAACACATTTATGAGG ATTTGGATTTTGAATTTGTATTTCCCTCTAAGTGCCTTTTTTAATGTCTATTTTTTTAATAA AACAGAAATGCATTCTTGTACAATTCTGTTGAAACTGGACCAAGGCTCTCAGAAGAGGA CCCCCGAGTTCCTTCCCCTCCCCCGAGCCTCTGCATGATTGTTTCAAGTCAGCCTGGA ATTCTTACTTTCACGCCGCTATTCTTTTCCTTTCTCCGTGATTGCTTGGCTAGCCATTTA AAAAAAAATATTCTCTGTTCAGTGTATATGTTGCTTGTTTGTTTTATTTATTGAGATATTTT TACAAGCTAAGTGACTGCAGTGTGGCTGTGTATCCTGCTCCCCACCCAGGAAAAATAAA GACGTCCGCGCA. (SEQ ID NO:234; NM_001271893.4), or a nucleic acid sequence that hybridizes to a nucleic acid sequence consisting of SEQ ID NO:234 under stringent hybridization conditions

In some embodiments, MAGE family member D1 (MAGED1, NRAGE, DLXIN-1) comprises the amino acid sequence: MAQKMDCGAGLLGFQAEASVEDSALLMQTLMEAIQISEAPPTNQATAAASPQSSQPPTAN EMADIQVSAAAARPKSAFKVQNATTKGPNGVYDFSQAHNAKDVPNTQPKAAFKSQNATPK GPNAAYDFSQAATTGELAANKSEMAFKAQNATTKVGPNATYNFSQSLNANDLANSRPKTP FKAWNDTTKAPTADTQTQNVNQAKMATSQADIETDPGISEPDGATAQTSADGSQAQNLES RTIIRGKRTRKINNLNVEENSSGDQRRAPLAAGTWRSAPVPVTTQNPPGAPPNVLWQTPLA WQNPSGWQNQTARQTPPARQSPPARQTPPAWQNPVAWQNPVIWPNPVIWQNPVIWPN PIVWPGPVVWPNPLAWQNPPGWQTPPGWQTPPGWQGPPDWQGPPDWPLPPDWPLPP DWPLPTDWPLPPDWIPADWPIPPDWQNLRPSPNLRPSPNSRASQNPGAAQPRDVALLQE RANKLVKYLMLKDYTKVPIKRSEMLRDIIREYTDVYPEIIERACFVLEKKFGIQLKEIDKEEHLY ILISTPESLAGILGTTKDTPKLGLLLVILGVIFMNGNRASEAVLWEALRKMGLRPGVRHPLLG DLRKLLTYEFVKQKYLDYRRVPNSNPPEYEFLWGLRSYHETSKMKVLRFIAEVQKRDPRD WTAQFMEAADEALDALDAAAAEAEARAEARTRMGIGDEAVSGPWSWDDIEFELLTWDEE GDFGDPWSRIPFTFWARYHQNARSRFPQTFAGPIIGPGGTASANFAANFGAIGFFWVE. (SEQ ID NO:235; NP_001005332.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:235)

In some embodiments, the nucleic acid sequence encoding MAGED1 comprises the nucleic acid sequence: AGCACTTCCGGTCACGCCATCGTTGCTGCCTTCTTCGTCAAGCCCCCAGGCTCCGCTC TTGCCAGAGGGACAGGAGCCATGGCTCAGAAAATGGACTGTGGTGCGGGCCTCCTCG GCTTCCAGGCTGAGGCCTCCGTAGAAGACAGCGCCTTGCTTATGCAGACCTTGATGGA GGCCATCCAGATCTCAGAGGCTCCACCTACTAACCAGGCCACCGCAGCTGCTAGTCCC CAGAGTTCACAGCCCCCAACTGCCAATGAGATGGCTGACATTCAGGTTTCAGCAGCTG CCGCTAGGCCTAAGTCAGCCTTTAAAGTCCAGAATGCCACCACAAAAGGCCCAAATGG TGTCTATGATTTCTCTCAGGCTCATAATGCCAAGGATGTGCCCAACACGCAGCCCAAG GCAGCCTTTAAGTCCCAAAATGCTACCCCAAAGGGTCCAAATGCTGCCTATGATTTTTC CCAGGCAGCAACCACTGGTGAGTTAGCTGCTAACAAGTCTGAGATGGCCTTCAAGGCC CAGAATGCCACTACTAAAGTGGGCCCAAATGCCACCTACAATTTCTCTCAGTCTCTCAA TGCCAATGACCTGGCCAACAGCAGGCCTAAGACCCCTTTCAAGGCTTGGAATGATACC ACTAAGGCCCCAACAGCTGATACCCAGACCCAGAATGTAAATCAGGCCAAAATGGCCA CTTCCCAGGCTGACATAGAGACCGACCCAGGTATCTCTGAACCTGACGGTGCAACTGC ACAGACATCAGCAGATGGTTCCCAGGCTCAGAATCTGGAGTCCCGGACAATAATTCGG GGCAAGAGGACCCGCAAGATTAATAACTTGAATGTTGAAGAGAACAGCAGTGGGGATC AGAGGCGGGCCCCACTGGCTGCAGGGACCTGGAGGTCTGCACCAGTTCCAGTGACCA CTCAGAACCCACCTGGCGCACCCCCCAATGTGCTCTGGCAGACGCCATTGGCTTGGC AGAACCCCTCAGGCTGGCAAAACCAGACAGCCAGGCAGACCCCACCAGCACGTCAGA GCCCTCCAGCTAGGCAGACCCCACCAGCCTGGCAGAACCCAGTCGCTTGGCAGAACC CAGTGATTTGGCCAAACCCAGTAATCTGGCAGAACCCAGTGATCTGGCCAAACCCCAT TGTCTGGCCCGGCCCTGTTGTCTGGCCGAATCCACTGGCCTGGCAGAATCCACCTGG ATGGCAGACTCCACCTGGATGGCAGACCCCACCGGGCTGGCAGGGTCCTCCAGACTG GCAAGGTCCTCCTGACTGGCCGCTACCACCCGACTGGCCACTGCCACCTGATTGGCC ACTTCCCACTGACTGGCCACTACCACCTGACTGGATCCCCGCTGATTGGCCAATTCCA CCTGACTGGCAGAACCTGCGCCCCTCGCCTAACCTGCGCCCTTCTCCCAACTCGCGTG CCTCACAGAACCCAGGTGCTGCACAGCCCCGAGATGTGGCCCTTCTTCAGGAAAGAG CAAATAAGTTGGTCAAGTACTTGATGCTTAAGGACTACACAAAGGTGCCCATCAAGCGC TCAGAAATGCTGAGAGATATCATCCGTGAATACACTGATGTTTATCCAGAAATCATTGAA CGTGCATGCTTTGTCCTAGAGAAGAAATTTGGGATTCAACTGAAAGAAATTGACAAAGA AGAACACCTGTATATTCTCATCAGTACCCCCGAGTCCCTGGCTGGCATACTGGGAACG ACCAAAGACACACCCAAGCTCGGTCTCCTCTTGGTGATTCTGGGTGTCATCTTCATGAA TGGCAACCGTGCCAGTGAGGCTGTCCTCTGGGAGGCACTACGCAAGATGGGACTGCG TCCTGGGGTGAGACATCCCCTCCTTGGAGATCTAAGGAAACTTCTCACCTATGAGTTTG TAAAGCAGAAATACCTGGACTACAGACGAGTGCCCAACAGCAACCCCCCGGAGTATGA GTTCCTCTGGGGCCTCCGTTCCTACCATGAGACTAGCAAGATGAAAGTGCTGAGATTC ATTGCAGAGGTTCAGAAAAGAGACCCTCGTGACTGGACTGCACAGTTCATGGAGGCTG CAGATGAGGCCTTGGATGCTCTGGATGCTGCTGCAGCTGAGGCCGAAGCCCGGGCTG AAGCAAGAACCCGCATGGGAATTGGAGATGAGGCTGTGTCTGGGCCCTGGAGCTGGG ATGACATTGAGTTTGAGCTGCTGACCTGGGATGAGGAAGGAGATTTTGGAGATCCCTG GTCCAGAATTCCATTTACCTTCTGGGCCAGATACCACCAGAATGCCCGCTCCAGATTCC CTCAGACCTTTGCCGGTCCCATTATTGGTCCTGGTGGTACAGCCAGTGCCAACTTCGC TGCCAACTTTGGTGCCATTGGTTTCTTCTGGGTTGAGTGAGATGTTGGATATTGCTATC AATCGCAGTAGTCTTTCCCCTGTGTGAGGCTGAAGCCTCAGATTCCTTCTAAACACAGC TATCTAGAGAGCCACATCCTGTTGACTGAAAGTGGCATGCAAGATAAATTTATTTGCTG TTCCTTGTCTACTGCTTTTTTTCCCCTTGTGTGCTGTCAAGTTTTGGTATCAGAAATAAA CATTGAAATTGCAAAGTGAA. (SEQ ID NO:236; NM_001005332.2), or a nucleic acid sequence that hybridizes to a nucleic acid sequence consisting of SEQ ID NO:236 under stringent hybridization conditions

In some embodiments, SATB homeobox 2 (SATB2, GLSS) comprises the amino acid sequence: MERRSESPCLRDSPDRRSGSPDVKGPPPVKVARLEQNGSPMGARGRPNGAVAKAVGGL MIPVFCVVEQLDGSLEYDNREEHAEFVLVRKDVLFSQLVETALLALGYSHSSAAQAQGIIKL GRWNPLPLSYVTDAPDATVADMLQDVYHVVTLKIQLQSCSKLEDLPAEQWNHATVRNALK ELLKEMNQSTLAKECPLSQSMISSIVNSTYYANVSATKCQEFGRWYKKYKKIKVERVEREN LSDYCVLGQRPMHLPNMNQLASLGKTNEQSPHSQIHHSTPIRNQVPALQPIMSPGLLSPQL SPQLVRQQIAMAHLINQQIAVSRLLAHQHPQAINQQFLNHPPIPRAVKPEPTNSSVEVSPDIY QQVRDELKRASVSQAVFARVAFNRTQGLLSEILRKEEDPRTASQSLLVNLRAMQNFLNLPE VERDRIYQDERERSMNPNVSMVSSASSSPSSSRTPQAKTSTPTTDLPIKVDGANINITAAIY DEIQQEMKRAKVSQALFAKVAANKSQGWLCELLRWKENPSPENRTLWENLCTIRRFLNLP QHERDVIYEEESRHHHSERMQHVVQLPPEPVQVLHRQQSQPAKESSPPREEAPPPPPPT EDSCAKKPRSRTKISLEALGILQSFIHDVGLYPDQEAIHTLSAQLDLPKHTIIKFFQNQRYHVK HHGKLKEHLGSAVDVAEYKDEELLTESEENDSEEGSEEMYKVEAEEENADKSKAAPAEID QR. (SEQ ID NO:237; NP_001165980.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:237)

In some embodiments, the nucleic acid sequence encoding SATB2 comprises the nucleic acid sequence: GAGTCCGGCTCTGGCTGCTGGCAGAGGCGGCCGAGAGGGGAGAGGCTGGAGGTGAC AGCTTGGGCGGCCGCCGCGTTTCCTCCCGCGCGCGGTCCCGGGTCCCTGCGTCTTCT CGGCTCTTGGTGTTACCGGTCCCACCGCTCTGGCCGCGCCTCCTCGCGAGCTAGCCG CCCTGCGAACCAGCAGCCCCGGCTCGCCGCCGCCGCCGCCGCCTCCGGGTTCTCAG CCCTTTCTCTCCAGAACGGGTCTCCTTCCCGAAGGTGTGAAAAGGCTCTTTCAGCCTC CTTCTCTTCCCCCCTCCTCCGCCGTCCCCTCCCCCGCTCGCTCGGGTGTCCCTTTGGA GGAGTCCTTTCCCTCTCCTCCTCCTCCCCCTCCTCCCTCCCCCCATCATCATCATAACA ACCATCTCCGCACCAGAAGAAGACACCCTGACCCAGGACCTTAAACATTAGGACCTGG GGAAGAGGGAAGGGGAAGGAGTAAAGAGGAAGACTAGGAGAACACTGCAAAGCCAAG CACCAGAAACTTTCCACCCTGGATTCTCTACTTTTGCTCCATGGACAGAGCCCCAGTCA GCCAAGTTTCAGACAGACCGTGAGCAGTCCCTGTGCGTTTTATTGCGACCTGCCGGTG GGAACTTTGTCTCCGAGTCGGAGCAGCATGGAGCGGCGGAGCGAGAGCCCGTGTCTG CGGGACAGCCCCGACCGGCGGAGCGGCAGCCCGGACGTCAAGGGGCCTCCCCCAGT GAAGGTGGCCCGGCTGGAGCAGAACGGCAGCCCCATGGGAGCCCGCGGGAGGCCCA ACGGCGCCGTGGCCAAGGCCGTGGGAGGTTTGATGATTCCTGTCTTTTGTGTCGTGGA GCAGTTGGACGGCTCTCTTGAATATGACAACAGAGAAGAACACGCCGAGTTTGTCCTG GTGCGGAAAGATGTGCTTTTTAGCCAGCTGGTGGAGACTGCGCTCCTGGCCCTGGGG TATTCTCACAGCTCTGCGGCCCAGGCCCAAGGAATAATCAAGCTGGGAAGGTGGAACC CTCTCCCCCTCAGTTATGTGACAGATGCACCCGACGCGACAGTGGCCGACATGCTACA AGATGTCTATCATGTTGTGACGTTGAAAATCCAATTACAAAGTTGTTCAAAGTTGGAAGA CTTGCCTGCGGAGCAGTGGAACCATGCCACAGTCCGCAATGCCTTAAAGGAACTGCTC AAAGAGATGAACCAGAGCACATTAGCCAAAGAATGCCCTCTCTCCCAGAGTATGATTTC ATCCATTGTAAATAGCACATATTATGCCAATGTGTCAGCAACCAAGTGCCAGGAGTTTG GGAGATGGTATAAAAAGTACAAGAAGATTAAAGTGGAAAGAGTGGAACGAGAAAACCTT TCAGACTATTGTGTTCTGGGCCAGCGTCCAATGCATTTACCAAATATGAACCAGCTGGC ATCCCTGGGGAAAACCAACGAACAGTCTCCTCACAGCCAAATTCACCACAGTACTCCAA TCCGAAACCAAGTGCCCGCATTACAGCCCATCATGAGCCCTGGTCTTCTTTCTCCCCA GCTTAGTCCACAACTTGTAAGGCAACAAATAGCCATGGCCCATCTGATAAACCAACAGA TTGCCGTTAGCCGGCTCCTGGCTCACCAGCATCCTCAAGCCATCAACCAGCAGTTCCT GAACCATCCACCCATCCCCAGAGCAGTTAAGCCAGAGCCAACCAACTCTTCCGTGGAA GTCTCTCCAGATATCTACCAGCAAGTCAGAGATGAGCTGAAGAGGGCCAGTGTGTCCC AAGCTGTCTTTGCAAGAGTGGCATTCAACCGCACACAGGGATTGTTGTCTGAGATTCTG CGTAAGGAAGAAGACCCTCGGACAGCCTCTCAGTCTCTTCTAGTAAACCTGAGGGCCA TGCAGAATTTCCTCAATCTGCCAGAAGTGGAGCGAGATCGCATCTACCAGGATGAGAG GGAGCGGAGCATGAATCCCAATGTGAGCATGGTCTCCTCGGCCTCCAGCAGTCCCAG CTCCTCCCGAACCCCTCAGGCCAAAACCTCGACACCGACAACAGACCTCCCTATTAAG GTGGACGGCGCCAACATCAACATCACAGCTGCCATTTATGACGAGATCCAACAGGAGA TGAAAAGGGCCAAGGTGTCTCAAGCCCTGTTTGCCAAAGTGGCTGCAAATAAAAGTCA GGGCTGGCTGTGTGAACTGCTCCGCTGGAAGGAGAACCCAAGCCCAGAAAACCGCAC CCTCTGGGAAAACCTCTGTACCATCCGTCGCTTCCTGAACCTTCCCCAGCATGAGAGG GATGTCATCTATGAGGAGGAGTCAAGGCATCACCACAGCGAACGCATGCAACACGTGG TCCAGCTTCCCCCTGAGCCGGTGCAGGTACTTCATAGACAGCAGTCTCAGCCAGCCAA GGAGAGTTCCCCTCCCAGAGAAGAAGCGCCTCCCCCACCTCCTCCGACTGAAGACAG TTGTGCCAAAAAGCCCCGGTCTCGCACAAAGATCTCCTTAGAAGCCCTGGGGATCCTC CAAAGCTTTATTCATGATGTAGGCCTGTACCCAGACCAGGAAGCCATCCACACTCTTTC GGCTCAGCTGGATCTCCCCAAACACACCATCATCAAGTTCTTCCAGAACCAGCGGTAC CACGTGAAGCACCACGGGAAGCTGAAAGAGCACCTGGGCTCCGCGGTGGACGTGGCT GAATATAAGGACGAGGAGCTGCTGACCGAGTCAGAGGAGAACGACAGCGAGGAAGGC TCCGAGGAGATGTACAAAGTGGAGGCTGAGGAGGAAAATGCTGACAAAAGCAAGGCA GCACCTGCCGAAATTGACCAGAGATAATGTGAACTTCTACTAGGCAAAGCAATACATCG GTCCAAGGATTTTCTGCTTTCATTTCTTTAAAAGTTTTTTGTTAGTTTGTTTTTTGTTTTTG TTTTTGGGTTTTTTTGGCTTTATTTTTGTCTTTTTATGTCTGTTTTGTTTTTCTTACCCTTT TGGACATTTCTTTGTTGCACAGGATACACCTATAGACTGAATAAGTTCAGTATTTCCGAA TCAGACATCGCCTTGGCAAAGACACTAAAGCGTTACACTTTATCCCGTCTCTATGACTG GATCATAGTCATTATAATCACAGGAGACTCTGCCTTCATTATCCTTGCACTTAACGGAAG TTACATCAGGCAAGTACCAGGATGAAAAGAACTATGAAATAAATGAAGGAAGCTACAAG TGTGTGTGTATATGTATATGTATATATCTCTATATTTACATATATATATTAAAATTGCATG GGACAGAGACTTTGCAATCCGAAAGAATAGACTGTGAAATGAGTTCTTAAAGAAAAGAC TTGTTTATGTATTAAAAAAACCACTTCACAGTGAGTCGCTTTGGCTTTTTGATAAACTGC GGCCTGCTCTCAGGGTGGGGTGACTATTTTTGAATTCCTATTTATTTTTTGTGTTTGTCC CTGATTTTTTTTTTTTAATTCTATGGCTTCCTATCTGGCAGCTTAATGGGTAATTTTTGAG GTATGTATTTAACAAAATAAACGACACTGCCGAAAAAAAAAAAAGTGAAGTGAAAACAAT CAGGGCACATTAAAATGATACAAGTCAAATAAATCTTAAAGACACAATGCACACTTAAAA TGACTCAATAAAATGACTTGCTACGTTCCGTTATTCAATTTGTCATTACTGTAGTGAACA GATGCATTTCTGTGGAATTCCAAATAAGTAAAACTGAAATTCAGTGCAGAGAAAACTTTG TCCACTAGTGCAAGTCTTGATCAAATGACATTTTGACATTGGACATATGGAATTCATAGT ATGAGCCACATTTTGTTGTGAAATTTATTTACCTGCTTGTGGCTTCAAATCTGAAAATTA ATAAGCCTGCTCGTTTAAAAGTTGTTTGTTGTTGCTGTTTTTTTGTCTTTTTGTTTTTTAC TAGAAAATAGTTCAGTGTAATATTAAGTTAGAAAAGAAGTTGCTGCCCAGTTAAAGGGG CTCCCTCTCAAATAAATCTCCATCCTTCCCTCTCCCAAAAGACATTTCTGATTTCTGCTT CACTTTGGGCTTCCTCTTCTTCGTACACATTCCATCTACCTAATCAAACATTTTCAGTCC CTGATCTCTCCTGTCCCTTTTCCTGGGATGACAGCCCTAACAAGAACTGTTTTTGAATC GTTGTGCAGCTCCAGGCAATAGAGTATGTGAAGCGATTTCAGTAGAATCACTTACTCAT CCTAAAAGAAAACATTATCCCAGTTACCTACATCGCAATTACCTTATGTAAAGCAGAACT AATGCTGACTGGATGTTTAATGGGATGAGCATTAAAGCTGCAATCTACTATAGTACTCC AGATCTCTTTCGGCTTCCTATGAGAAACACCAGAAGCATTACTTTCCACTTCTACTTACA GTAATTGCAAGAGGAGACCTCACATTCAGGACTGCCTAGTGAACGTAATCCATGCTTTA AACTGGCCATTAAACAGTCCCACATGGTTGGATTTTTTTTTTTTTTTTGAGTTGTGCTTTC ACAAAACCTTGTCAAAGACCTCATGCAATATCACTTTGAAAGTTATTTTCTGTTTACTACA CAAACATTGTAATATAACTGTTAATACTATTTATATATTTGAAAGGTATAAAAGGTAGGAG TTAAAAAAAAAACCTCTATGTGTAGATATTAACTCAGAACTTACAATATACAGGGAGAAG ACATGTTGCAATACAAGCTAATTCTAGCTGCTCAGTAACCTCTGGAGTTTTTAAAGGGA CATTTTCCTGTACTTTTTCAAATAATGATGTTTAAAAATTATCTTGACATAAGCGTCATAT ACCTTTGCAAAAGGATGGTTGTTTGCAGTTAGCCCTGGCCCCATCCTTCCTATTTCTGT AGTATGCTGCAGCTTTAATCAGAAAGTCCATGGTTGCTGCTTCCTGATCTCCGAGTTAC TCTTTCCAAATTGTCTTCTTACACTGTTGCTGAAGGTCACTCTGTACACGTAATGGAAAC TGATTTTGCCAAGCTCTTACAAGGTGGTTCATCTATCGATGGCATCCGCATTTGGTATC TTTTACACTTCAACCAAAAATTTATTAGGTATTTTTCAATGCTAAGTCTTGCCTTTTATTTT TTAATTTCACTGCCAAGTTTGCAGTGGTTCTAAGTGAATCTGTGGGCATTTTAGCCTGT GGTCTTGCCAGATCTTTGCGAATTACAATGCATATATGTCTATTTATTCAATATCTGTCAT ATAATATCTATTTGGAAGAAGAAACTTTCTCTTGTAGTGCCTCTTGACAAAGCACAATTT CCCGCCTTTTTTTTTTTTTGTGAAATGAAAAAAACAAATTGTGTTTTATTGCGGTATCAAC AATGTGAATAAGGATTAACATATTGTAAATGTTCTTTTTTCCATGTAAATCAACTATCTTT GTTATCACTAAGTGATAATTAATTTTTAACTTATGTGCATTGTTAGGCTGTTAGAATTTTT TGGTTGTTAAAATAAACGCATTCAATAAA. (SEQ ID NO:238; NM_001172509.2), or a nucleic acid sequence that hybridizes to a nucleic acid sequence consisting of SEQ ID NO:238 under stringent hybridization conditions

In some embodiments, PDZ and LIM domain 7 (LMP3, PDLIM7, LMP1) comprises the amino acid sequence: MDSFKVVLEGPAPWGFRLQGGKDFNVPLSISRLTPGGKAAQAGVAVGDWVLSIDGENAG SLTHIEAQNKIRACGERLSLGLSRAQPVQSKPQKASAPAADPPRYTFAPSVSLNKTARPFG APPPADSAPQQNGQPLRPLVPDASKQRLMENTEDWRPRPGTGQSRSFRILAHLTGTEFM QDPDEEHLKKSSQVPRTEAPAPASSTPQEPWPGPTAPSPTSRPPWAVDPAFAERYAPDK TSTVLTRHSQPATPTPLQSRTSIVQAAAGGVPGGGSNNGKTPVCHQCHKVIRGRYLVALG HAYHPEEFVCSQCGKVLEEGGFFEEKGAIFCPPCYDVRYAPSCAKCKKKITGEIMHALKMT WHVHCFTCAACKTPIRNRAFYMEEGVPYCERDYEKMFGTKCHGCDFKIDAGDRFLEALGF SWHDTCFVCAICQINLEGKTFYSKKDRPLCKSHAFSHV (SEQ ID NO:239; NP_005442.2), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:239)

In some embodiments, the nucleic acid sequence encoding LMP3 comprises the nucleic acid sequence: GTCAGAACACTGGCGGCCGATCCCAACGAGGCTCCCTGGAGCCCGACGCAGAGCAGC GCCCTGGCCGGGCCAAGCAGGAGCCGGCATCATGGATTCCTTCAAAGTAGTGCTGGA GGGGCCAGCACCTTGGGGCTTCCGGCTGCAAGGGGGCAAGGACTTCAATGTGCCCCT CTCCATTTCCCGGCTCACTCCTGGGGGCAAAGCGGCGCAGGCCGGAGTGGCCGTGG GTGACTGGGTGCTGAGCATCGATGGCGAGAATGCGGGTAGCCTCACACACATCGAAG CTCAGAACAAGATCCGGGCCTGCGGGGAGCGCCTCAGCCTGGGCCTCAGCAGGGCC CAGCCGGTTCAGAGCAAACCGCAGAAGGCCTCCGCCCCCGCCGCGGACCCTCCGCG GTACACCTTTGCACCCAGCGTCTCCCTCAACAAGACGGCCCGGCCCTTTGGGGCGCC CCCGCCCGCTGACAGCGCCCCGCAGCAGAATGGACAGCCGCTCCGACCGCTGGTCC CAGATGCCAGCAAGCAGCGGCTGATGGAGAACACAGAGGACTGGCGGCCGCGGCCG GGGACAGGCCAGTCGCGTTCCTTCCGCATCCTTGCCCACCTCACAGGCACCGAGTTCA TGCAAGACCCGGATGAGGAGCACCTGAAGAAATCAAGCCAGGTGCCCAGGACAGAAG CCCCAGCCCCAGCCTCATCTACACCCCAGGAGCCCTGGCCTGGCCCTACCGCCCCCA GCCCTACCAGCCGCCCGCCCTGGGCTGTGGACCCTGCGTTTGCCGAGCGCTATGCCC CGGACAAAACGAGCACAGTGCTGACCCGGCACAGCCAGCCGGCCACGCCCACGCCG CTGCAGAGCCGCACCTCCATTGTGCAGGCAGCTGCCGGAGGGGTGCCAGGAGGGGG CAGCAACAACGGCAAGACTCCCGTGTGTCACCAGTGCCACAAGGTCATCCGGGGCCG CTACCTGGTGGCGCTGGGCCACGCGTACCACCCGGAGGAGTTTGTGTGTAGCCAGTG TGGGAAGGTCCTGGAAGAGGGTGGCTTCTTTGAGGAGAAGGGCGCCATCTTCTGCCC ACCATGCTATGACGTGCGCTATGCACCCAGCTGTGCCAAGTGCAAGAAGAAGATTACA GGCGAGATCATGCACGCCCTGAAGATGACCTGGCACGTGCACTGCTTTACCTGTGCTG CCTGCAAGACGCCCATCCGGAACAGGGCCTTCTACATGGAGGAGGGCGTGCCCTATT GCGAGCGAGACTATGAGAAGATGTTTGGCACGAAATGCCATGGCTGTGACTTCAAGAT CGACGCTGGGGACCGCTTCCTGGAGGCCCTGGGCTTCAGCTGGCATGACACCTGCTT CGTCTGTGCGATATGTCAGATCAACCTGGAAGGAAAGACCTTCTACTCCAAGAAGGAC AGGCCTCTCTGCAAGAGCCATGCCTTCTCTCATGTGTGAGCCCCTTCTGCCCACAGCT GCCGCGGTGGCCCCTAGCCTGAGGGGCCTGGAGTCGTGGCCCTGCATTTCTGGGTAG GGCTGGCAATGGTTGCCTTAACCCTGGCTCCTGGCCCGAGCCTGGGGCTCCCTGGGC CCTGCCCCACCCACCTTATCCTCCCACCCCACTCCCTCCACCACCACAGCACACCGGT GCTGGCCACACCAGCCCCCTTTCACCTCCAGTGCCACAATAAACCTGTACCCAGCTGT G. (SEQ ID NO:240; NM_005451.5), or a nucleic acid sequence that hybridizes to a nucleic acid sequence consisting of SEQ ID NO:240 under stringent hybridization conditions

In some embodiments, POU class 5 homeobox 1 (OCT3, OCT4, POU5F1) comprises the amino acid sequence: MGVLFGKVFSQTTICRFEALQLSFKNMCKLRPLLQKWVEEADNNENLQEICKAETLVQARK RKRTSIENRVRGNLENLFLQCPKPTLQQISHIAQQLGLEKDVVRVWFCNRRQKGKRSSSDY AQREDFEAAGSPFSGGPVSFPLAPGPHFGTPGYGSPHFTALYSSVPFPEGEAFPPVSVTT LGSPMHSN. (SEQ ID NO:241; NP_001167002.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:241)

In some embodiments, the nucleic acid sequence encoding OCT3 comprises the nucleic acid sequence: GGAAAAAAGGAAAGTGCACTTGGAAGAGATCCAAGTGGGCAACTTGAAGAACAAGTGC CAAATAGCACTTCTGTCATGCTGGATGTCAGGGCTCTTTGTCCACTTTGTATAGCCGCT GGCTTATAGAAGGTGCTCGATAAATCTCTTGAATTTAAAAATCAATTAGGATGCCTCTAT AGTGAAAAAGATACAGTAAAGATGAGGGATAATCAATTTAAAAAATGAGTAAGTACACA CAAAGCACTTTATCCATTCTTATGACACCTGTTACTTTTTTGCTGTGTTTGTGTGTATGC ATGCCATGTTATAGTTTGTGGGACCCTCAAAGCAAGCTGGGGAGAGTATATACTGAATT TAGCTTCTGAGACATGATGCTCTTCCTTTTTAATTAACCCAGAACTTAGCAGCTTATCTA TTTCTCTAATCTCAAAACATCCTTAAACTGGGGGTGATACTTGAGTGAGAGAATTTTGCA GGTATTAAATGAACTATCTTCTTTTTTTTTTTTCTTTGAGACAGAGTCTTGCTCTGTCACC CAGGCTGGAGTGCAGTGGCGTGATCTCAGCTCACTGCAACCTCCGCCTCCCGGGTTC AAGTGATTCTCCTGCCTCAGCCTCCTGAGTAGCTGGGATTACAGTCCCAGGACATCAA AGCTCTGCAGAAAGAACTCGAGCAATTTGCCAAGCTCCTGAAGCAGAAGAGGATCACC CTGGGATATACACAGGCCGATGTGGGGCTCACCCTGGGGGTTCTATTTGGGAAGGTAT TCAGCCAAACGACCATCTGCCGCTTTGAGGCTCTGCAGCTTAGCTTCAAGAACATGTGT AAGCTGCGGCCCTTGCTGCAGAAGTGGGTGGAGGAAGCTGACAACAATGAAAATCTTC AGGAGATATGCAAAGCAGAAACCCTCGTGCAGGCCCGAAAGAGAAAGCGAACCAGTAT CGAGAACCGAGTGAGAGGCAACCTGGAGAATTTGTTCCTGCAGTGCCCGAAACCCACA CTGCAGCAGATCAGCCACATCGCCCAGCAGCTTGGGCTCGAGAAGGATGTGGTCCGA GTGTGGTTCTGTAACCGGCGCCAGAAGGGCAAGCGATCAAGCAGCGACTATGCACAA CGAGAGGATTTTGAGGCTGCTGGGTCTCCTTTCTCAGGGGGACCAGTGTCCTTTCCTC TGGCCCCAGGGCCCCATTTTGGTACCCCAGGCTATGGGAGCCCTCACTTCACTGCACT GTACTCCTCGGTCCCTTTCCCTGAGGGGGAAGCCTTTCCCCCTGTCTCCGTCACCACT CTGGGCTCTCCCATGCATTCAAACTGAGGTGCCTGCCCTTCTAGGAATGGGGGACAGG GGGAGGGGAGGAGCTAGGGAAAGAAAACCTGGAGTTTGTGCCAGGGTTTTTGGGATT AAGTTCTTCATTCACTAAGGAAGGAATTGGGAACACAAAGGGTGGGGGCAGGGGAGTT TGGGGCAACTGGTTGGAGGGAAGGTGAAGTTCAATGATGCTCTTGATTTTAATCCCACA TCATGTATCACTTTTTTCTTAAATAAAGAAGCCTGGGACACAGTAGATAGACACACTTAA AAAAAAAAA. (SEQ ID NO:242; NM_001173531.2), or a nucleic acid sequence that hybridizes to a nucleic acid sequence consisting of SEQ ID NO:242 under stringent hybridization conditions

In some embodiments, Kruppel like factor 4 (KLF4, EZF, GKLF) comprises the amino acid sequence: MRQPPGESDMAVSDALLPSFSTFASGPAGREKTLRQAGAPNNRWREELSHMKRLPPVLP GRPYDLAAATVATDLESGGAGAACGGSNLAPLPRRETEEFNDLLDLDFILSNSLTHPPESV AATVSSSASASSSSSPSSSGPASAPSTCSFTYPIRAGNDPGVAPGGTGGGLLYGRESAPP PTAPFNLADINDVSPSGGFVAELLRPELDPVYIPPQQPQPPGGGLMGKFVLKASLSAPGSE YGSPSVISVSKGSPDGSHPVVVAPYNGGPPRTCPKIKQEAVSSCTHLGAGPPLSNGHRPA AHDFPLGRQLPSRTTPTLGLEEVLSSRDCHPALPLPPGFHPHPGPNYPSFLPDQMQPQVP PLHYQGQSRGFVARAGEPCVCWPHFGTHGMMLTPPSSPLELMPPGSCMPEEPKPKRGR RSWPRKRTATHTCDYAGCGKTYTKSSH LKAHLRTHTGEKPYHCDWDGCGWKFARSDELT RHYRKHTGHRPFQCQKCDRAFSRSDHLALHMKRHF. (SEQ ID NO:243; NP_001300981.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:243)

In some embodiments, the nucleic acid sequence encoding KLF4 comprises the nucleic acid sequence: GGCAGTTTCCCGACCAGAGAGAACGAACGTGTCTGCGGGCGCGCGGGGAGCAGAGG CGGTGGCGGGCGGCGGCGGCACCGGGAGCCGCCGAGTGACCCTCCCCCGCCCCTCT GGCCCCCCACCCTCCCACCCGCCCGTGGCCCGCGCCCATGGCCGCGCGCGCTCCAC ACAACTCACCGGAGTCCGCGCCTTGCGCCGCCGACCAGTTCGCAGCTCCGCGCCACG GCAGCCAGTCTCACCTGGCGGCACCGCCCGCCCACCGCCCCGGCCACAGCCCCTGC GCCCACGGCAGCACTCGAGGCGACCGCGACAGTGGTGGGGGACGCTGCTGAGTGGA AGAGAGCGCAGCCCGGCCACCGGACCTACTTACTCGCCTTGCTGATTGTCTATTTTTG CGTTTACAACTTTTCTAAGAACTTTTGTATACAAAGGAACTTTTTAAAAAAGACGCTTCCA AGTTATATTTAATCCAAAGAAGAAGGATCTCGGCCAATTTGGGGTTTTGGGTTTTGGCT TCGTTTCTTCTCTTCGTTGACTTTGGGGTTCAGGTGCCCCAGCTGCTTCGGGCTGCCG AGGACCTTCTGGGCCCCCACATTAATGAGGCAGCCACCTGGCGAGTCTGACATGGCT GTCAGCGACGCGCTGCTCCCATCTTTCTCCACGTTCGCGTCTGGCCCGGCGGGAAGG GAGAAGACACTGCGTCAAGCAGGTGCCCCGAATAACCGCTGGCGGGAGGAGCTCTCC CACATGAAGCGACTTCCCCCAGTGCTTCCCGGCCGCCCCTATGACCTGGCGGCGGCG ACCGTGGCCACAGACCTGGAGAGCGGCGGAGCCGGTGCGGCTTGCGGCGGTAGCAA CCTGGCGCCCCTACCTCGGAGAGAGACCGAGGAGTTCAACGATCTCCTGGACCTGGA CTTTATTCTCTCCAATTCGCTGACCCATCCTCCGGAGTCAGTGGCCGCCACCGTGTCCT CGTCAGCGTCAGCCTCCTCTTCGTCGTCGCCGTCGAGCAGCGGCCCTGCCAGCGCGC CCTCCACCTGCAGCTTCACCTATCCGATCCGGGCCGGGAACGACCCGGGCGTGGCGC CGGGCGGCACGGGCGGAGGCCTCCTCTATGGCAGGGAGTCCGCTCCCCCTCCGACG GCTCCCTTCAACCTGGCGGACATCAACGACGTGAGCCCCTCGGGCGGCTTCGTGGCC GAGCTCCTGCGGCCAGAATTGGACCCGGTGTACATTCCGCCGCAGCAGCCGCAGCCG CCAGGTGGCGGGCTGATGGGCAAGTTCGTGCTGAAGGCGTCGCTGAGCGCCCCTGG CAGCGAGTACGGCAGCCCGTCGGTCATCAGCGTCAGCAAAGGCAGCCCTGACGGCAG CCACCCGGTGGTGGTGGCGCCCTACAACGGCGGGCCGCCGCGCACGTGCCCCAAGA TCAAGCAGGAGGCGGTCTCTTCGTGCACCCACTTGGGCGCTGGACCCCCTCTCAGCA ATGGCCACCGGCCGGCTGCACACGACTTCCCCCTGGGGCGGCAGCTCCCCAGCAGG ACTACCCCGACCCTGGGTCTTGAGGAAGTGCTGAGCAGCAGGGACTGTCACCCTGCC CTGCCGCTTCCTCCCGGCTTCCATCCCCACCCGGGGCCCAATTACCCATCCTTCCTGC CCGATCAGATGCAGCCGCAAGTCCCGCCGCTCCATTACCAAGGTCAGTCCCGGGGAT TTGTAGCTCGGGCTGGGGAGCCCTGTGTGTGCTGGCCCCACTTCGGGACACACGGGA TGATGCTCACCCCACCTTCTTCACCCCTAGAGCTCATGCCACCCGGTTCCTGCATGCC AGAGGAGCCCAAGCCAAAGAGGGGAAGACGATCGTGGCCCCGGAAAAGGACCGCCA CCCACACTTGTGATTACGCGGGCTGCGGCAAAACCTACACAAAGAGTTCCCATCTCAA GGCACACCTGCGAACCCACACAGGTGAGAAACCTTACCACTGTGACTGGGACGGCTG TGGATGGAAATTCGCCCGCTCAGATGAACTGACCAGGCACTACCGTAAACACACGGGG CACCGCCCGTTCCAGTGCCAAAAATGCGACCGAGCATTTTCCAGGTCGGACCACCTCG CCTTACACATGAAGAGGCATTTTTAAATCCCAGACAGTGGATATGACCCACACTGCCAG AAGAGAATTCAGTATTTTTTACTTTTCACACTGTCTTCCCGATGAGGGAAGGAGCCCAG CCAGAAAGCACTACAATCATGGTCAAGTTCCCAACTGAGTCATCTTGTGAGTGGATAAT CAGGAAAAATGAGGAATCCAAAAGACAAAAATCAAAGAACAGATGGGGTCTGTGACTG GATCTTCTATCATTCCAATTCTAAATCCGACTTGAATATTCCTGGACTTACAAAATGCCA AGGGGGTGACTGGAAGTTGTGGATATCAGGGTATAAATTATATCCGTGAGTTGGGGGA GGGAAGACCAGAATTCCCTTGAATTGTGTATTGATGCAATATAAGCATAAAAGATCACC TTGTATTCTCTTTACCTTCTAAAAGCCATTATTATGATGTTAGAAGAAGAGGAAGAAATT CAGGTACAGAAAACATGTTTAAATAGCCTAAATGATGGTGCTTGGTGAGTCTTGGTTCT AAAGGTACCAAACAAGGAAGCCAAAGTTTTCAAACTGCTGCATACTTTGACAAGGAAAA TCTATATTTGTCTTCCGATCAACATTTATGACCTAAGTCAGGTAATATACCTGGTTTACTT CTTTAGCATTTTTATGCAGACAGTCTGTTATGCACTGTGGTTTCAGATGTGCAATAATTT GTACAATGGTTTATTCCCAAGTATGCCTTAAGCAGAACAAATGTGTTTTTCTATATAGTT CCTTGCCTTAATAAATATGTAATATAAATTTAAGCAAACGTCTATTTTGTATATTTGTAAA CTACAAAGTAAAATGAACATTTTGTGGAGTTTGTATTTTGCATACTCAAGGTGAGAATTA AGTTTTAAATAAACCTATAATATTTTATCTGAA. (SEQ ID NO:244; NM_001314052.2), or a nucleic acid sequence that hybridizes to a nucleic acid sequence consisting of SEQ ID NO:244 under stringent hybridization conditions

In some embodiments, MYC proto-oncogene, bHLH transcription factor (MYC, MRTL, MYCC, c-MC) comprises the amino acid sequence: MDFFRVVENQPPATMPLNVSFTNRNYDLDYDSVQPYFYCDEEENFYQQQQQSELQPPAP SEDIWKKFELLPTPPLSPSRRSGLCSPSYVAVTPFSLRGDNDGGGGSFSTADQLEMVTELL GGDMVNQSFICDPDDETFIKNIIIQDCMWSGFSAAAKLVSEKLASYQAARKDSGSPNPARG HSVCSTSSLYLQDLSAAASECIDPSVVFPYPLNDSSSPKSCASQDSSAFSPSSDSLLSSTES SPQGSPEPLVLHEETPPTTSSDSEEEQEDEEEIDVVSVEKRQAPGKRSESGSPSAGGHSK PPHSPLVLKRCHVSTHQHNYAAPPSTRKDYPAAKRVKLDSVRVLRQISNNRKCTSPRSSDT EENVKRRTHNVLERQRRNELKRSFFALRDQIPELENNEKAPKVVILKKATAYILSVQAEEQK LISEEDLLRKRREQLKHKLEQLRNSCA. (SEQ ID NO:245; NP_001341799.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:245)

In some embodiments, the nucleic acid sequence encoding MYC comprises the nucleic acid sequence: GGAGTTTATTCATAACGCGCTCTCCAAGTATACGTGGCAATGCGTTGCTGGGTTATTTT AATCATTCTAGGCATCGTTTTCCTCCTTATGCCTCTATCATTCCTCCCTATCTACACTAA CATCCCACGCTCTGAACGCGCGCCCATTAATACCCTTCTTTCCTCCACTCTCCCTGGGA CTCTTGATCAAAGCGCGGCCCTTTCCCCAGCCTTAGCGAGGCGCCCTGCAGCCTGGTA CGCGCGTGGCGTGGCGGTGGGCGCGCAGTGCGTTCTCGGTGTGGAGGGCAGCTGTT CCGCCTGCGATGATTTATACTCACAGGACAAGGATGCGGTTTGTCAAACAGTACTGCTA CGGAGGAGCAGCAGAGAAAGGGAGAGGGTTTGAGAGGGAGCAAAAGAAAATGGTAGG CGCGCGTAGTTAATTCATGCGGCTCTCTTACTCTGTTTACATCCTAGAGCTAGAGTGCT CGGCTGCCCGGCTGAGTCTCCTCCCCACCTTCCCCACCCTCCCCACCCTCCCCATAAG CGCCCCTCCCGGGTTCCCAAAGCAGAGGGCGTGGGGGAAAAGAAAAAAGATCCTCTC TCGCTAATCTCCGCCCACCGGCCCTTTATAATGCGAGGGTCTGGACGGCTGAGGACCC CCGAGCTGTGCTGCTCGCGGCCGCCACCGCCGGGCCCCGGCCGTCCCTGGCTCCCC TCCTGCCTCGAGAAGGGCAGGGCTTCTCAGAGGCTTGGCGGGAAAAAGAACGGAGGG AGGGATCGCGCTGAGTATAAAAGCCGGTTTTCGGGGCTTTATCTAACTCGCTGTAGTAA TTCCAGCGAGAGGCAGAGGGAGCGAGCGGGCGGCCGGCTAGGGTGGAAGAGCCGG GCGAGCAGAGCTGCGCTGCGGGCGTCCTGGGAAGGGAGATCCGGAGCGAATAGGGG GCTTCGCCTCTGGCCCAGCCCTCCCGCTGATCCCCCAGCCAGCGGTCCGCAACCCTT GCCGCATCCACGAAACTTTGCCCATAGCAGCGGGCGGGCACTTTGCACTGGAACTTAC AACACCCGAGCAAGGACGCGACTCTCCCGACGCGGGGAGGCTATTCTGCCCATTTGG GGACACTTCCCCGCCGCTGCCAGGACCCGCTTCTCTGAAAGGCTCTCCTTGCAGCTGC TTAGACGCTGGATTTTTTTCGGGTAGTGGAAAACCAGCCTCCCGCGACGATGCCCCTC AACGTTAGCTTCACCAACAGGAACTATGACCTCGACTACGACTCGGTGCAGCCGTATTT CTACTGCGACGAGGAGGAGAACTTCTACCAGCAGCAGCAGCAGAGCGAGCTGCAGCC CCCGGCGCCCAGCGAGGATATCTGGAAGAAATTCGAGCTGCTGCCCACCCCGCCCCT GTCCCCTAGCCGCCGCTCCGGGCTCTGCTCGCCCTCCTACGTTGCGGTCACACCCTT CTCCCTTCGGGGAGACAACGACGGCGGTGGCGGGAGCTTCTCCACGGCCGACCAGCT GGAGATGGTGACCGAGCTGCTGGGAGGAGACATGGTGAACCAGAGTTTCATCTGCGA CCCGGACGACGAGACCTTCATCAAAAACATCATCATCCAGGACTGTATGTGGAGCGGC TTCTCGGCCGCCGCCAAGCTCGTCTCAGAGAAGCTGGCCTCCTACCAGGCTGCGCGC AAAGACAGCGGCAGCCCGAACCCCGCCCGCGGCCACAGCGTCTGCTCCACCTCCAGC TTGTACCTGCAGGATCTGAGCGCCGCCGCCTCAGAGTGCATCGACCCCTCGGTGGTC TTCCCCTACCCTCTCAACGACAGCAGCTCGCCCAAGTCCTGCGCCTCGCAAGACTCCA GCGCCTTCTCTCCGTCCTCGGATTCTCTGCTCTCCTCGACGGAGTCCTCCCCGCAGGG CAGCCCCGAGCCCCTGGTGCTCCATGAGGAGACACCGCCCACCACCAGCAGCGACTC TGAGGAGGAACAAGAAGATGAGGAAGAAATCGATGTTGTTTCTGTGGAAAAGAGGCAG GCTCCTGGCAAAAGGTCAGAGTCTGGATCACCTTCTGCTGGAGGCCACAGCAAACCTC CTCACAGCCCACTGGTCCTCAAGAGGTGCCACGTCTCCACACATCAGCACAACTACGC AGCGCCTCCCTCCACTCGGAAGGACTATCCTGCTGCCAAGAGGGTCAAGTTGGACAGT GTCAGAGTCCTGAGACAGATCAGCAACAACCGAAAATGCACCAGCCCCAGGTCCTCGG ACACCGAGGAGAATGTCAAGAGGCGAACACACAACGTCTTGGAGCGCCAGAGGAGGA ACGAGCTAAAACGGAGCTTTTTTGCCCTGCGTGACCAGATCCCGGAGTTGGAAAACAA TGAAAAGGCCCCCAAGGTAGTTATCCTTAAAAAAGCCACAGCATACATCCTGTCCGTCC AAGCAGAGGAGCAAAAGCTCATTTCTGAAGAGGACTTGTTGCGGAAACGACGAGAACA GTTGAAACACAAACTTGAACAGCTACGGAACTCTTGTGCGTAAGGAAAAGTAAGGAAAA CGATTCCTTCTAACAGAAATGTCCTGAGCAATCACCTATGAACTTGTTTCAAATGCATGA TCAAATGCAACCTCACAACCTTGGCTGAGTCTTGAGACTGAAAGATTTAGCCATAATGT AAACTGCCTCAAATTGGACTTTGGGCATAAAAGAACTTTTTTATGCTTACCATCTTTTTTT TTTCTTTAACAGATTTGTATTTAAGAATTGTTTTTAAAAAATTTTAAGATTTACACAATGTT TCTCTGTAAATATTGCCATTAAATGTAAATAACTTTAATAAAACGTTTATAGCAGTTACAC AGAATTTCAATCCTAGTATATAGTACCTAGTATTATAGGTACTATAAACCCTAATTTTTTT TATTTAAGTACATTTTGCTTTTTAAAGTTGATTTTTTTCTATTGTTTTTAGAAAAAATAAAA TAACTGGCAAATATATCATTGAGCCAAATCTTAAGTTGTGAATGTTTTGTTTCGTTTCTTC CCCCTCCCAACCACCACCATCCCTGTTTGTTTTCATCAATTGCCCCTTCAGAGGGTGGT CTTAAGAAAGGCAAGAGTTTTCCTCTGTTGAAATGGGTCTGGGGGCCTTAAGGTCTTTA AGTTCTTGGAGGTTCTAAGATGCTTCCTGGAGACTATGATAACAGCCAGAGTTGACAGT TAGAAGGAATGGCAGAAGGCAGGTGAGAAGGTGAGAGGTAGGCAAAGGAGATACAAG AGGTCAAAGGTAGCAGTTAAGTACACAAAGAGGCATAAGGACTGGGGAGTTGGGAGG AAGGTGAGGAAGAAACTCCTGTTACTTTAGTTAACCAGTGCCAGTCCCCTGCTCACTCC AAACCCAGGAATTCTGCCCAGTTGATGGGGACACGGTGGGAACCAGCTTCTGCTGCCT TCACAACCAGGCGCCAGTCCTGTCCATGGGTTATCTCGCAAACCCCAGAGGATCTCTG GGAGGAATGCTACTATTAACCCTATTTCACAAACAAGGAAATAGAAGAGCTCAAAGAGG TTATGTAACTTATCTGTAGCCACGCAGATAATACAAAGCAGCAATCTGGACCCATTCTG TTCAAAACACTTAACCCTTCGCTATCATGCCTTGGTTCATCTGGGTCTAATGTGCTGAG ATCAAGAAGGTTTAGGACCTAATGGACAGACTCAAGTCATAACAATGCTAAGCTCTATT TGTGTCCCAAGCACTCCTAAGCATTTTATCCCTAACTCTACATCAACCCCATGAAGGAG ATACTGTTGATTTCCCCATATTAGAAGTAGAGAGGGAAGCTGAGGCACACAAAGACTCA TCCACATGCCCAAGATTCACTGATAGGGAAAAGTGGAAGCGAGATTTGAACCCAGGCT GTTTACTCCTAACCTGTCCAAGCCACCTCTCAGACGACGGTAGGAATCAGCTGGCTGC TTGTGAGTACAGGAGTTACAGTCCAGTGGGTTATGTTTTTTAAGTCTCAACATCTAAGC CTGGTCAGGCATCAGTTCCCCTTTTTTTGTGATTTATTTTGTTTTTATTTTGTTGTTCATT GTTTAATTTTTCCTTTTACAATGAGAAGGTCACCATCTTGACTCCTACCTTAGCCATTTG TTGAATCAGACTCATGACGGCTCCTGGGAAGAAGCCAGTTCAGATCATAAAATAAAACA TATTTATTCTTTGTCATGGGAGTCATTATTTTAGAAACTACAAACTCTCCTTGCTTCCATC CTTTTTTACATACTCATGACACATGCTCATCCTGAGTCCTTGAAAAGGTATTTTTGAACA TGTGTATTAATTATAAGCCTCTGAAAACCTATGGCCCAAACCAGAAATGATGTTGATTAT ATAGGTAAATGAAGGATGCTATTGCTGTTCTAATTACCTCATTGTCTCAGTCTCAAAGTA GGTCTTCAGCTCCCTGTACTTTGGGATTTTAATCTACCACCACCCATAAATCAATAAATA ATTACTTTCTTTGA. (SEQ ID NO:246; NM_001354870.1), or a nucleic acid sequence that hybridizes to a nucleic acid sequence consisting of SEQ ID NO:246 under stringent hybridization conditions

In some embodiments, distal-less homeobox 3 (DLX3, A14, TDO) comprises the amino acid sequence: MSGSFDRKLSSILTDISSSLSCHAGSKDSPTLPESSVTDLGYYSAPQHDYYSGQPYGQTVN PYTYHHQFNLNGLAGTGAYSPKSEYTYGASYRQYGAYREQPLPAQDPVSVKEEPEAEVR MVNGKPKKVRKPRTIYSSYQLAALQRRFQKAQYLALPERAELAAQLGLTQTQVKIWFQNRR SKFKKLYKNGEVPLEHSPNNSDSMACNSPPSPALWDTSSHSTPAPARSQLPPPLPYSASP SYLDDPTNSWYHAQNLSGPHLQQQPPQPATLHHASPGPPPNPGAVY (SEQ ID NO:247; NP_005211.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:247)

In some embodiments, the nucleic acid sequence encoding DLX3 comprises the nucleic acid sequence: AGCATTTGATTGTGGCTTGGGACGCGAGGAGAGGCGCGCAGCGACCGCCTGACGGCA GGCAATGGTGTAAGCGCCTCTCGGCCTCCCCCTCCCCCCAGACGCGGCCGGGTCCTC CCTTCGCCTTCTGGACACACACCCCTGCCTCGTCTCTTCCGCCTCTCTCGCACTCCGG TCCGTTCCTGTCCTCTGCGGAGGCCAGCCCTGGGGAGGTGCAGCGCCCGCCAGGATG AGTGGCTCCTTCGATCGCAAGCTCAGCAGCATCCTCACCGACATCTCCAGCTCCCTTA GCTGCCATGCGGGCTCCAAGGACTCGCCTACCCTGCCCGAGTCTTCTGTCACTGACCT GGGCTACTACAGCGCTCCCCAGCACGATTACTACTCGGGCCAGCCCTATGGCCAGAC GGTGAACCCCTACACCTACCACCACCAATTCAATCTCAATGGGCTTGCAGGCACGGGC GCTTACTCGCCCAAGTCGGAATATACCTACGGAGCCTCCTACCGGCAATACGGGGCGT ATCGGGAGCAGCCGCTGCCAGCCCAGGACCCAGTGTCGGTGAAGGAGGAGCCGGAA GCAGAGGTGCGCATGGTGAATGGGAAGCCCAAGAAGGTCCGAAAGCCGCGTACGATC TACTCCAGCTACCAGCTGGCCGCCCTGCAGCGCCGCTTCCAGAAGGCCCAGTACCTG GCGCTGCCCGAGCGCGCCGAGCTGGCCGCGCAGCTGGGCCTCACGCAGACACAGGT GAAAATCTGGTTCCAGAACCGCCGTTCCAAGTTCAAGAAACTCTACAAGAACGGGGAG GTGCCGCTGGAGCACAGTCCCAATAACAGTGATTCCATGGCCTGCAACTCACCACCAT CACCCGCCCTCTGGGACACCTCTTCCCACTCCACTCCGGCCCCTGCCCGCAGTCAGC TGCCCCCGCCGCTCCCATACAGTGCCTCCCCCAGCTACCTGGACGACCCCACCAACT CCTGGTATCACGCACAGAACCTGAGTGGACCCCACTTACAGCAGCAGCCGCCTCAGC CAGCCACCCTGCACCATGCCTCTCCCGGGCCCCCGCCCAACCCTGGGGCTGTGTACT GAGCACCCATCTGGCCTGCACCCTTGACAAAGGACCCCAGGACCAGGCAGAAGGCGC CTCCGTCCTAGCCACTCAGGAATCATCGAGGAGCACAGGGAAAAGGAACTCCCTTTCC CCCTCCCTTGCCCCTTCCTCCAGGGACCCAAGCGCTTCCAGATGACAATTGCATGGAC CAAGGATGCCCCCTGAACCTCCCTCCCTCTGCCTAGACACTGGGGTACCCCTCCAGAT GTGGGGACATTCCACCCCAGTGGGGACAGCCATTCCCCTACCTGCTCCAGGAGCCTG GATTGGCTTTAAATGGCTCATCATCTTCCAGCTTCTTAAACTTAGTGCCTGTTCCCAGAC TGGAGACCTTGGGATGGGGGAGAGTGTGGAGGGTTTGCGGGTCCTGCCTGTGCTGGG GCACCTGGCACCGTGGATCTTAAAACTTGCCAGGCCTAGTTCCTCCTGAGCCTCTGGT GGTCTCCCCCTGCTCGAGCGGCCCCTCGGCCAATAAGACAGTGGACATCATGACGAG GACTCCGGGTGGGGACCTGAACTGGTCACCGCCCTGCACTTCTAGCCCTCATTTAAGA TTTGAGGGTGAAACCAAAGAAAACCCCCTAAGTGAGGGAATCTTTTAATATTTGTGGCT TTAGAGGAAAGAACTAAAGGAGCCATCTCTCTCCCCTCTCCTCCGTTCCGAGAGGAGG GGTGGGTCTCAGACGTTTTTCCTATGGACTTATTTCTTCCATGTCCAGGACTTTGCACA ACTTTGGTTTTAAAAGCTGTTGAAAAATAGGAAAACAAAGGGCATTGTTCACAGATAGG GCCAAGTCTCCCCTTGCAAGGGTGCCTCTGTTCTGTCCCTGCCCCCACCTCACCTTCT CTACTCCTCCAGTAAGTTGGCAGTTTTGGTGCCAAACCCCAAATCTCCAAAGAGACATA CCAGGCAAGACAAACCCCCAAACACCTCCTTTCCGGTGGCCTTGGAAACAGATTGCTC CGAGCTGGAGAATGTCGGGTGAGGTGTATGGGAGAGGAGGGGAGAGTTAGAACTTGT GCCTTTGGGAGTAAGGGGTAACTGCCTGGAGGGCTGGTGGCACTGCCCCTCCCTGAC CCAGACATCCCACCAAAGCTAACTTTCCCCCACCCCTGATGCAGTAAAACATTGAAAAA AAAAAAAAAGGAGAGGTAGAAGACTGTAGCTATATATATAAATATATAGTAAGTTTTTTTT TTTTAAGAGCAACAGAGAGAAGCAGCCTCCTCCCTGCTGCGGTTTCCTATTTATGTGGC CATGTTCCTCCTGGACGGATCTCCCTGTGTGTTTCAAGCTGAGAGATGTGGGCTCCGG CTGGATTTGGGTTTTGTGGGAGGTGCAGGGGCCAAGAGAGACGTGGTAGGTCTCCAA GAGTCCCACCCGGGGGGGAAGAAGCAAAGCCATCTCCCACCCCCTCCCAGCCTTCTC ATTTCTGCTTTCTTACTGGACTCATCTTTATATATAATGTTAATAAAAAAGACGAAAATAA. (SEQ ID NO:248; NM_005220.3), or a nucleic acid sequence that hybridizes to a nucleic acid sequence consisting of SEQ ID NO:248 under stringent hybridization conditions

In some embodiments, distal-less homeobox 5 (DLX5, SHFM1D) comprises the amino acid sequence: MTGVFDRRVPSIRSGDFQAPFQTSAAMHHPSQESPTLPESSATDSDYYSPTGGAPHGYCS PTSASYGKALNPYQYQYHGVNGSAGSYPAKAYADYSYASSYHQYGGAYNRVPSATNQPE KEVTEPEVRMVNGKPKKVRKPRTIYSSFQLAALQRRFQKTQYLALPERAELAASLGLTQTQ VKIWFQNKRSKIKKIMKNGEMPPEHSPSSSDPMACNSPQSPAVWEPQGSSRSLSHHPHA HPPTSNQSPASSYLENSASWYTSAASSINSHLPPPGSLQHPLALASGTLY. (SEQ ID NO:249; NP_005212.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:249)

In some embodiments, the nucleic acid sequence encoding DLX5 comprises the nucleic acid sequence: AGCAGTCAGCCGGCCGGAGACAGAGACTTCACGACTCCCAGTCTCCTCCTCGCCGCG GCCGCCGCCTCCTCCTTCTCTCCTCCTCCTCTTCCTCCTCCTCCCTCGCTCCCACAGC CATGTCTGCTTAGACCAGAGCAGCCCCACAGCCAACTAGGGCAGCTGCCGCCGCCAC AACAGCAAGGACAGCCGCTGCCGCCGCCCGTGAGCGATGACAGGAGTGTTTGACAGA AGGGTCCCCAGCATCCGATCCGGCGACTTCCAAGCTCCGTTCCAGACGTCCGCAGCT ATGCACCATCCGTCTCAGGAATCGCCAACTTTGCCCGAGTCTTCAGCTACCGATTCTGA CTACTACAGCCCTACGGGGGGAGCCCCGCACGGCTACTGCTCTCCTACCTCGGCTTC CTATGGCAAAGCTCTCAACCCCTACCAGTATCAGTATCACGGCGTGAACGGCTCCGCC GGGAGCTACCCAGCCAAAGCTTATGCCGACTATAGCTACGCTAGCTCCTACCACCAGT ACGGCGGCGCCTACAACCGCGTCCCAAGCGCCACCAACCAGCCAGAGAAAGAAGTGA CCGAGCCCGAGGTGAGAATGGTGAATGGCAAACCAAAGAAAGTTCGTAAACCCAGGAC TATTTATTCCAGCTTTCAGCTGGCCGCATTACAGAGAAGGTTTCAGAAGACTCAGTACC TCGCCTTGCCGGAACGCGCCGAGCTGGCCGCCTCGCTGGGATTGACACAAACACAGG TGAAAATCTGGTTTCAGAACAAAAGATCCAAGATCAAGAAGATCATGAAAAACGGGGAG ATGCCCCCGGAGCACAGTCCCAGCTCCAGCGACCCAATGGCGTGTAACTCGCCGCAG TCTCCAGCGGTGTGGGAGCCCCAGGGCTCGTCCCGCTCGCTCAGCCACCACCCTCAT GCCCACCCTCCGACCTCCAACCAGTCCCCAGCGTCCAGCTACCTGGAGAACTCTGCAT CCTGGTACACAAGTGCAGCCAGCTCAATCAATTCCCACCTGCCGCCGCCGGGCTCCTT ACAGCACCCGCTGGCGCTGGCCTCCGGGACACTCTATTAGATGGGCTGCTCTCTCTTA CTCTCTTTTTTGGGACTACTGTGTTTTGCTGTTCTAGAAAATCATAAAGAAAGGAATTCA TATGGGGAAGTTCGGAAAACTGAAAAAGATTCATGTGTAAAGCTTTTTTTTGCATGTAAG TTATTGCATTTCAAAAGACCCCCCCTTTTTTTACAGAGGACTTTTTTTGCGCAACTGTGG ACACTTTCAATGGTGCCTTGAAATCTATGACCTCAACTTTTCAAAAGACTTTTTTCAATGT TATTTTAGCCATGTAAATAAGTGTAGATAGAGGAATTAAACTGTATATTCTGGATAAATA AAATTATTTCGACCATGAAAA. (SEQ ID NO:250; NM_005221.6), or a nucleic acid sequence that hybridizes to a nucleic acid sequence consisting of SEQ ID NO:250 under stringent hybridization conditions

In some embodiments, distal-less homeobox 6 (DLX6) comprises the amino acid sequence: MMTMTTMADGLEGQDSSKSAFMEFGQQQQQQQQQQQQQQQQQQQPPPPPPPPPQPH SQQSSPAMAGAHYPLHCLHSAAAAAAAGSHHHHHHQHHHHGSPYASGGGNSYNHRSLA AYPYMSHSQHSPYLQSYHNSSAAAQTRGDDTDQQKTTVIENGEIRFNGKGKKIRKPRTIYS SLQLQALNHRFQQTQYLALPERAELAASLGLTQTQVKIWFQNKRSKFKKLLKQGSNPHESD PLQGSAALSPRSPALPPVWDVSASAKGVSMPPNSYMPGYSHWYSSPHQDTMQRPQMM. (SEQ ID NO:251; NP_005213.3), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:251)

In some embodiments, the nucleic acid sequence encoding DLX6 comprises the nucleic acid sequence: ATTCGACTCGTGGCGTCTGCATCAAGTCTGAAAGCAGACGCGCAACTTTCGCAGAATC CACCTTAAAATCTCTGCCTTAAACTGCACCAGCCCCCAAAAAATCCAAGGGGGGAAAG CAGGCGGGGGGAGAGCAGATTCCCCCCTCCCCCTCTCCTCTCCCATCCCTCCTCCTTC CTCCTCCCTTTGAGTTAACAAGGCCCCGCTCACTATATCTCTTTATATTAAATATATATAT ATATTAGAGAAGAGCGAGGGAGAGGGAGAACCACCTCCACCCCCCTCTTTAAATTCTTT TTTTTTTTTTTTTTTTTTTTTGCAAGGATCCAAAGAGCTAAGGTGGCTGCAGAGGGGAGA GCGGCGCGAGCCAAGTGGGGGAGGGTGGAGGAAACCCGGGAGAAGGCTTTCTCCAG CCCCCAAAGTTTTTGATGATGACCATGACTACGATGGCTGACGGCTTGGAAGGCCAGG ACTCGTCCAAATCCGCCTTCATGGAGTTCGGGCAGCAGCAGCAGCAGCAGCAGCAAC AGCAGCAGCAGCAGCAGCAGCAACAGCAACAGCCGCCGCCGCCGCCGCCGCCGCCG CCGCAGCCGCACTCGCAGCAGAGCTCCCCGGCCATGGCAGGCGCGCACTACCCTCTG CACTGCCTGCACTCGGCGGCGGCGGCGGCAGCGGCCGGCTCGCACCACCACCACCA CCACCAGCACCACCACCACGGCTCGCCCTACGCGTCGGGCGGAGGGAACTCCTACAA CCACCGCTCGCTCGCCGCCTACCCCTACATGAGCCACTCGCAGCACAGCCCTTACCTC CAGTCCTACCACAACAGCAGCGCAGCCGCCCAGACGCGAGGGGACGACACAGATCAA CAAAAAACTACAGTGATTGAAAACGGGGAAATCAGGTTCAATGGAAAAGGGAAAAAGAT TCGGAAGCCTCGGACCATTTATTCCAGCCTGCAGCTCCAGGCTTTAAACCATCGCTTTC AGCAGACACAGTATCTGGCCCTTCCAGAGAGAGCCGAACTGGCAGCTTCCTTAGGACT GACACAAACACAGGTGAAGATATGGTTTCAGAACAAACGCTCTAAGTTTAAGAAACTGC TGAAGCAGGGCAGTAATCCTCATGAGAGCGACCCCCTCCAGGGCTCGGCGGCCCTGT CGCCACGCTCGCCAGCGCTGCCTCCAGTCTGGGACGTTTCTGCCTCGGCCAAGGGTG TCAGTATGCCCCCCAACAGCTACATGCCTGGCTATTCTCACTGGTACTCCTCTCCACAC CAGGACACGATGCAGAGACCACAGATGATGTGAGTTGCCCAAGGGAACACCCTAGGG AAACGTCTGAACAAGGAAAAGAGGATCCGGGACCTGCTTGTATCTGCGAAAAGGAGCC AAAGGAGCAGGCTTAGGAGAGCTCATAAGTGTGGCAAGAAGCCGACTAGGCTCATTCT CTCTCCCTCTCTCTCTCTCTCCCTCTCCTTTCTTTTTACTTCTTCCTTTCCTCCATTCCTT CTTTCTTTCCTTTTCCTTTCTACCTTTCTTTTCTTTTTGCCTTTCACCTTTTTTCTCATTTA CCTTCTCTCTTGAGCAACGTCAGTAATTGATCTTGCATCTCAGAGAGAGAGAAAGAGCA TGTGTGAGAGAGAAACTGGTTTCTATGCCAGCACTCCTGAAACCCCTTACTGTAAGGAT ATTTTCTCTTACCCCTTGGGATCCAGGCTCTGAGTCTCTTCTCTTTGGGAGTATCCATCA AAATGACTTTTTTTAAAAACAGATTTTCCCCCAACCAGAAGAATCTGCACAAACTTGGCA GCGTTTTTACTTGTTTAATGAGTTTAAGACATTACATGGTGAAAGAGAAGCATTTTGGAC TCCTGCATTTTTATTTACCATTCCCAGACTGACGAGAAAAAGAAAATTCCTCACATAACA GCCCTTCTCTAAAGAAAAAGGAAAAAGTGGCTTTGATTAAAAAAAAACAAAACAAAAACC ACTCTTTCCCCACCCCACCCCCCCAAACCCTGAACTGGAATCAGGAAAGACGGAGGAA ACAATCAAAATCACCATTCTATTGCTTTGACACCTTTACTAGGTGAATTGGTGGCATTCA CAAAGCTAATAGGGACGTTTATATCAAGAAACATTTCTGTATATATTGTTGAATTTTAGTT GTACATATACTTTGTATGTTTTTGTCTTCTTTCATATATGGAGTAAAAGCCACAAAACGCT GA. (SEQ ID NO:252; NM_005222.4), or a nucleic acid sequence that hybridizes to a nucleic acid sequence consisting of SEQ ID NO:252 under stringent hybridization conditions

In some embodiments, HOP homeobox2 (OB1, HOD, HOPX, LAGY, TOTO, CAMEO, NECC1, SMAP31) comprises the amino acid sequence: MSAETASGPTEDQVEILEYNFNKVDKHPDSTTLCLIAAEAGLSEEETQKWFKQRLAKWRRS EGLPSECRSVTD. (SEQ ID NO:253; NP_001138931.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:253)

In some embodiments, the nucleic acid sequence encoding OB1 comprises the nucleic acid sequence: GAGACAGAAGGCCGCCTACCGGGGAGGCCGGAGGCCGGCTAGTCGCGGACTCGGGC GAACCCACCCTCGCGATCTGTCAAGTCTGTCCCCAGGGGAGGTCCCCCTTTCGGGAG GAAGTTTTTAAGGGGATTTCTCAAAATCACCCCCGCGCTTCCTTCACTCCTTCCTTAGA GCCGGAGGTCGGTGAGGGCCCGCGGAATCATCTATCTCGCCCCCGTCGCAGCGCGC AGGGACCATGTCGGCGGAGACCGCGAGCGGCCCCACAGAGGACCAGGTGGAAATCC TGGAGTACAACTTCAACAAGGTCGACAAGCACCCGGATTCCACCACGCTGTGCCTCAT CGCGGCCGAGGCAGGCCTTTCCGAGGAGGAGACCCAGAAATGGTTTAAGCAGCGCCT GGCAAAGTGGCGGCGCTCAGAAGGCCTGCCCTCAGAGTGCAGATCCGTCACAGACTA AGGAGATGGCAGGCATTGACAGCTTCACTCCATGAAGGCCATCTCTGTTTCTCTCCTCC GCTTAACCAAGCTGTTGTGGTTTTTCAGCATAGTGTTGTATGTTCCATTGCTAGCTGTCC TGCTGTTTAACACAGTGTTGTATTTTTTTTCTAAATGTACATAATTAGAAAAGAAAATAAC AATAGGAAGCTATGTGTATCTTCTGTGTAAAGCAGTGGCTTCACTGGAAAAATGGTGTG GCTAGCATTTCCCTTTGAGTCATGATGACAGATGGTGTGAAAACCATCTAAGTTTGCTTT TGACCATCACCTCCCAGTAGCAATTTGCTTTCATAATCCATTTAGCAATCCAGGCCTCT GTTGAAAAGATAATATGAGGGAGAAGGGAACACATTTCCTTCTGAACTTACTTCCCTAA GTCACTTTCCTTATGTATCATCTAATACAATGATGGTTGAGTGAAAATACAGAAGGGGT GTTTGAGTATTCAGATTTCATAAAACACTTCCTTGGAATATAGCTGCATTAACTTGGAAA GAAGCCTGTTGGGCCAGAAGACAGAAACTCCAACTGGCAAAAAAGCAAGCATCTAAGA AAAAAAACCACCAAAGTTCTTGAATTTACTATATTTAAATGCATTGGTTAAGTTTATTTTG CTAAATAAAGTGAACTGCTTTTTGTCTCTAAAATGATATTCTAAATAAAACCTTAACTTTT TGTTGAAGATGCACTGAAAAAAAAAA. (SEQ ID NO:254; NM_001145459.1), or a nucleic acid sequence that hybridizes to a nucleic acid sequence consisting of SEQ ID NO:254 under stringent hybridization conditions

In some embodiments, CCAAT enhancer binding protein alpha (CEBPA, CEBP C/EBP) comprises the amino acid sequence: MPGGAHGPPPGYGCAAAGYLDGRLEPLYERVGAPALRPLVIKQEPREEDEAKQLALAGLF PYQPPPPPPPSHPHPHPPPAHLAAPHLQFQIAHCGQTTMHLQPGHPTPPPTPVPSPHPAP ALGAAGLPGPGSALKGLGAAHPDLRASGGSGAGKAKKSVDKNSNEYRVRRERNNIAVRKS RDKAKQRNVETQQKVLELTSDNDRLRKRVEQLSRELDTLRGIFRQLPESSLVKAMGNCA. (SEQ ID NO:255; NP_001272758.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:255)

In some embodiments, the nucleic acid sequence encoding CEBP comprises the nucleic acid sequence: TATAAAAGCTGGGCCGGCGCGGGCCGGGCCATTCGCGACCCGGAGGTGCGCGGGCG CGGGCGAGCAGGGTCTCCGGGTGGGCGGCGGCGACGCCCCGCGCAGGCTGGAGGC CGCCGAGGCTCGCCATGCCGGGAGAACTCTAACTCCCCCATGGAGTCGGCCGACTTC TACGAGGCGGAGCCGCGGCCCCCGATGAGCAGCCACCTGCAGAGCCCCCCGCACGC GCCCAGCAGCGCCGCCTTCGGCTTTCCCCGGGGCGCGGGCCCCGCGCAGCCTCCCG CCCCACCTGCCGCCCCGGAGCCGCTGGGCGGCATCTGCGAGCACGAGACGTCCATC GACATCAGCGCCTACATCGACCCGGCCGCCTTCAACGACGAGTTCCTGGCCGACCTG TTCCAGCACAGCCGGCAGCAGGAGAAGGCCAAGGCGGCCGTGGGCCCCACGGGCGG CGGCGGCGGCGGCGACTTTGACTACCCGGGCGCGCCCGCGGGCCCCGGCGGCGCC GTCATGCCCGGGGGAGCGCACGGGCCCCCGCCCGGCTACGGCTGCGCGGCCGCCG GCTACCTGGACGGCAGGCTGGAGCCCCTGTACGAGCGCGTCGGGGCGCCGGCGCTG CGGCCGCTGGTGATCAAGCAGGAGCCCCGCGAGGAGGATGAAGCCAAGCAGCTGGC GCTGGCCGGCCTCTTCCCTTACCAGCCGCCGCCGCCGCCGCCGCCCTCGCACCCGC ACCCGCACCCGCCGCCCGCGCACCTGGCCGCCCCGCACCTGCAGTTCCAGATCGCG CACTGCGGCCAGACCACCATGCACCTGCAGCCCGGTCACCCCACGCCGCCGCCCACG CCCGTGCCCAGCCCGCACCCCGCGCCCGCGCTCGGTGCCGCCGGCCTGCCGGGCCC TGGCAGCGCGCTCAAGGGGCTGGGCGCCGCGCACCCCGACCTCCGCGCGAGTGGCG GCAGCGGCGCGGGCAAGGCCAAGAAGTCGGTGGACAAGAACAGCAACGAGTACCGG GTGCGGCGCGAGCGCAACAACATCGCGGTGCGCAAGAGCCGCGACAAGGCCAAGCA GCGCAACGTGGAGACGCAGCAGAAGGTGCTGGAGCTGACCAGTGACAATGACCGCCT GCGCAAGCGGGTGGAACAGCTGAGCCGCGAACTGGACACGCTGCGGGGCATCTTCC GCCAGCTGCCAGAGAGCTCCTTGGTCAAGGCCATGGGCAACTGCGCGTGAGGCGCGC GGCTGTGGGACCGCCCTGGGCCAGCCTCCGGCGGGGACCCAGGGAGTGGTTTGGGG TCGCCGGATCTCGAGGCTTGCCCGAGCCGTGCGAGCCAGGACTAGGAGATTCCGGTG CCTCCTGAAAGCCTGGCCTGCTCCGCGTGTCCCCTCCCTTCCTCTGCGCCGGACTTGG TGCGTCTAAGATGAGGGGGCCAGGCGGTGGCTTCTCCCTGCGAGGAGGGGAGAATTC TTGGGGCTGAGCTGGGAGCCCGGCAACTCTAGTATTTAGGATAACCTTGTGCCTTGGA AATGCAAACTCACCGCTCCAATGCCTACTGAGTAGGGGGAGCAAATCGTGCCTTGTCA TTTTATTTGGAGGTTTCCTGCCTCCTTCCCGAGGCTACAGCAGACCCCCATGAGAGAA GGAGGGGAGCAGGCCCGTGGCAGGAGGAGGGCTCAGGGAGCTGAGATCCCGACAAG CCCGCCAGCCCCAGCCGCTCCTCCACGCCTGTCCTTAGAAAGGGGTGGAAACATAGG GACTTGGGGCTTGGAACCTAAGGTTGTTCCCCTAGTTCTACATGAAGGTGGAGGGTCT CTAGTTCCACGCCTCTCCCACCTCCCTCCGCACACACCCCACCCCAGCCTGCTATAGG CTGGGCTTCCCCTTGGGGCGGAACTCACTGCGATGGGGGTCACCAGGTGACCAGTGG GAGCCCCCACCCCGAGTCACACCAGAAAGCTAGGTCGTGGGTCAGCTCTGAGGATGT ATACCCCTGGTGGGAGAGGGAGACCTAGAGATCTGGCTGTGGGGCGGGCATGGGGG GTGAAGGGCCACTGGGACCCTCAGCCTTGTTTGTACTGTATGCCTTCAGCATTGCCTA GGAACACGAAGCACGATCAGTCCATCCCAGAGGGACCGGAGTTATGACAAGCTTTCCA AATATTTTGCTTTATCAGCCGATATCAACACTTGTATCTGGCCTCTGTGCCCCAGCAGT GCCTTGTGCAATGTGAATGTGCGCGTCTCTGCTAAACCACCATTTTATTTGGTTTTTGTT TTGTTTTGGTTTTGCTCGGATACTTGCCAAAATGAGACTCTCCGTCGGCAGCTGGGGG AAGGGTCTGAGACTCCCTTTCCTTTTGGTTTTGGGATTACTTTTGATCCTGGGGGACCA ATGAGGTGAGGGGGGTTCTCCTTTGCCCTCAGCTTTCCCCAGCCCCTCCGGCCTGGG CTGCCCACAAGGCTTGTCCCCCAGAGGCCCTGGCTCCTGGTCGGGAAGGGAGGTGGC CTCCCGCCAACGCATCACTGGGGCTGGGAGCAGGGAAGGACGGCTTGGTTCTCTTCT TTTGGGGAGAACGTAGAGTCTCACTCTAGATGTTTTATGTATTATATCTATAATATAAAC ATATCAAAGTCAA. (SEQ ID NO:256; NM_001285829.1), or a nucleic acid sequence that hybridizes to a nucleic acid sequence consisting of SEQ ID NO:256 under stringent hybridization conditions

In some embodiments, activating transcription factor 4 (ATF4, CREB2, TXREB) comprises the amino acid sequence: MTEMSFLSSEVLVGDLMSPFDQSGLGAEESLGLLDDYLEVAKHFKPHGFSSDKAKAGSSE WLAVDGLVSPSNNSKEDAFSGTDWMLEKMDLKEFDLDALLGIDDLETMPDDLLTTLDDTC DLFAPLVQETNKQPPQTVNPIGHLPESLTKPDQVAPFTFLQPLPLSPGVLSSTPDHSFSLEL GSEVDITEGDRKPDYTAYVAMIPQCIKEEDTPSDNDSGICMSPESYLGSPQHSPSTRGSPN RSLPSPGVLCGSARPKPYDPPGEKMVAAKVKGEKLDKKLKKMEQNKTAATRYRQKKRAE QEALTGECKELEKKNEALKERADSLAKEIQYLKDLIEEVRKARGKKRVP. (SEQ ID NO:257; NP_001666.2), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:257)

In some embodiments, the nucleic acid sequence encoding ATF4 comprises the nucleic acid sequence: AGCCATTTCTACTTTGCCCGCCCACAGATGTAGTTTTCTCTGCGCGTGTGCGTTTTCCC TCCTCCCCGCCCTCAGGGTCCACGGCCACCATGGCGTATTAGGGGCAGCAGTGCCTG CGGCAGCATTGGCCTTTGCAGCGGCGGCAGCAGCACCAGGCTCTGCAGCGGCAACCC CCAGCGGCTTAAGCCATGGCGTGAGTACCGGGGCGGGTCGTCCAGCTGTGCTCCTGG GGCCGGCGCGGGTTTTGGATTGGTGGGGTGCGGCCTGGGGCCAGGGCGGTGCCGCC AAGGGGGAAGCGATTTAACGAGCGCCCGGGACGCGTGGTCTTTGCTTGGGTGTCCCC GAGACGCTCGCGTGCCTGGGATCGGGAAAGCGTAGTCGGGTGCCCGGACTGCTTCCC CAGGAGCCCTACAGCCCTCGGACCCCGAGCCCCGCAAGGGTCCCAGGGGTCTTGGCT GTTGCCCCACGAAACGTGGCAGGAACCAAGATGGCGGCGGCAGGGCGGCGGCGCGG GCGTGAGTCAAGGGCGGGCGGTGGGCGGGGCGCGGCCGCCCTGGCCGTATTTGGAC GTGGGGACGGAGCGCTTTCCTCTTGGCGGCCGGTGGAAGAATCCCCTGGTCTCCGTG AGCGTCCATTTTGTGGAACCTGAGTTGCAAGCAGGGAGGGGCAAATACAACTGCCCTG TTCCCGATTCTCTAGATGGCCGATCTAGAGAAGTCCCGCCTCATAAGTGGAAGGATGA AATTCTCAGAACAGCTAACCTCTAATGGGAGTTGGCTTCTGATTCTCATTCAGGCTTCT CACGGCATTCAGCAGCAGCGTTGCTGTAACCGACAAAGACACCTTCGAATTAAGCACA TTCCTCGATTCCAGCAAAGCACCGCAACATGACCGAAATGAGCTTCCTGAGCAGCGAG GTGTTGGTGGGGGACTTGATGTCCCCCTTCGACCAGTCGGGTTTGGGGGCTGAAGAA AGCCTAGGTCTCTTAGATGATTACCTGGAGGTGGCCAAGCACTTCAAACCTCATGGGTT CTCCAGCGACAAGGCTAAGGCGGGCTCCTCCGAATGGCTGGCTGTGGATGGGTTGGT CAGTCCCTCCAACAACAGCAAGGAGGATGCCTTCTCCGGGACAGATTGGATGTTGGAG AAAATGGATTTGAAGGAGTTCGACTTGGATGCCCTGTTGGGTATAGATGACCTGGAAAC CATGCCAGATGACCTTCTGACCACGTTGGATGACACTTGTGATCTCTTTGCCCCCCTAG TCCAGGAGACTAATAAGCAGCCCCCCCAGACGGTGAACCCAATTGGCCATCTCCCAGA AAGTTTAACAAAACCCGACCAGGTTGCCCCCTTCACCTTCTTACAACCTCTTCCCCTTT CCCCAGGGGTCCTGTCCTCCACTCCAGATCATTCCTTTAGTTTAGAGCTGGGCAGTGA AGTGGATATCACTGAAGGAGATAGGAAGCCAGACTACACTGCTTACGTTGCCATGATC CCTCAGTGCATAAAGGAGGAAGACACCCCTTCAGATAATGATAGTGGCATCTGTATGA GCCCAGAGTCCTATCTGGGGTCTCCTCAGCACAGCCCCTCTACCAGGGGCTCTCCAAA TAGGAGCCTCCCATCTCCAGGTGTTCTCTGTGGGTCTGCCCGTCCCAAACCTTACGAT CCTCCTGGAGAGAAGATGGTAGCAGCAAAAGTAAAGGGTGAGAAACTGGATAAGAAGC TGAAAAAAATGGAGCAAAACAAGACAGCAGCCACTAGGTACCGCCAGAAGAAGAGGGC GGAGCAGGAGGCTCTTACTGGTGAGTGCAAAGAGCTGGAAAAGAAGAACGAGGCTCT AAAAGAGAGGGCGGATTCCCTGGCCAAGGAGATCCAGTACCTGAAAGATTTGATAGAA GAGGTCCGCAAGGCAAGGGGGAAGAAAAGGGTCCCCTAGTTGAGGATAGTCAGGAGC GTCAATGTGCTTGTACATAGAGTGCTGTAGCTGTGTGTTCCAATAAATTATTTTGTAGGG AAAGTAAAAAAAAAAAAAAA (SEQ ID NO:258; NM_001675.4), or a nucleic acid sequence that hybridizes to a nucleic acid sequence consisting of SEQ ID NO:258 under stringent hybridization conditions

In some embodiments, SMAD family member 1 (SMAD1) comprises the amino acid sequence: MNVTSLFSFTSPAVKRLLGWKQGDEEEKWAEKAVDALVKKLKKKKGAMEELEKALSCPGQ PSNCVTIPRSLDGRLQVSHRKGLPHVIYCRVWRWPDLQSHHELKPLECCEFPFGSKQKEV CINPYHYKRVESPVLPPVLVPRHSEYNPQHSLLAQFRNLGQNEPHMPLNATFPDSFQQPN SHPFPHSPNSSYPNSPGSSSSTYPHSPTSSDPGSPFQMPADTPPPAYLPPEDPMTQDGS QPMDTNMMAPPLPSEINRGDVQAVAYEEPKHWCSIVYYELNNRVGEAFHASSTSVLVDGF TDPSNNKNRFCLGLLSNVNRNSTIENTRRHIGKGVHLYYVGGEVYAECLSDSSIFVQSRNC NYHHGFHPTTVCKIPSGCSLKIFNNQEFAQLLAQSVNHGFETVYELTKMCTIRMSFVKGWG AEYHRQDVTSTPCWIEIHLHGPLQWLDKVLTQMGSPHNPISSVS. (SEQ ID NO:259; NP_001003688.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:259)

In some embodiments, the nucleic acid sequence encoding SMAD1 comprises the nucleic acid sequence: CACTGCATGTGTATTCGTGAGTTCGCGGTTGAACAACTGTTCCTTTACTCTGCTCCCTG TCTTTGTGCTGACTGGGTTACTTTTTTAAACACTAGGAATGGTAATTTCTACTCTTCTGG ACTTCAAACTAAGAAGTTAAAGAGACTTCTCTGTAAATAAACAAATCTCTTCTGCTGTCC TTTTGCATTTGGAGACAGCTTTATTTCACCATATCCAAGGAGTATAACTAGTGCTGTCAT TATGAATGTGACAAGTTTATTTTCCTTTACAAGTCCAGCTGTGAAGAGACTTCTTGGGTG GAAACAGGGCGATGAAGAAGAAAAATGGGCAGAGAAAGCTGTTGATGCTTTGGTGAAA AAACTGAAGAAAAAGAAAGGTGCCATGGAGGAACTGGAAAAGGCCTTGAGCTGCCCAG GGCAACCGAGTAACTGTGTCACCATTCCCCGCTCTCTGGATGGCAGGCTGCAAGTCTC CCACCGGAAGGGACTGCCTCATGTCATTTACTGCCGTGTGTGGCGCTGGCCCGATCTT CAGAGCCACCATGAACTAAAACCACTGGAATGCTGTGAGTTTCCTTTTGGTTCCAAGCA GAAGGAGGTCTGCATCAATCCCTACCACTATAAGAGAGTAGAAAGCCCTGTACTTCCTC CTGTGCTGGTTCCAAGACACAGCGAATATAATCCTCAGCACAGCCTCTTAGCTCAGTTC CGTAACTTAGGACAAAATGAGCCTCACATGCCACTCAACGCCACTTTTCCAGATTCTTT CCAGCAACCCAACAGCCACCCGTTTCCTCACTCTCCCAATAGCAGTTACCCAAACTCTC CTGGGAGCAGCAGCAGCACCTACCCTCACTCTCCCACCAGCTCAGACCCAGGAAGCC CTTTCCAGATGCCAGCTGATACGCCCCCACCTGCTTACCTGCCTCCTGAAGACCCCAT GACCCAGGATGGCTCTCAGCCGATGGACACAAACATGATGGCGCCTCCCCTGCCCTC AGAAATCAACAGAGGAGATGTTCAGGCGGTTGCTTATGAGGAACCAAAACACTGGTGC TCTATTGTCTACTATGAGCTCAACAATCGTGTGGGTGAAGCGTTCCATGCCTCCTCCAC AAGTGTGTTGGTGGATGGTTTCACTGATCCTTCCAACAATAAGAACCGTTTCTGCCTTG GGCTGCTCTCCAATGTTAACCGGAATTCCACTATTGAAAACACCAGGCGGCATATTGGA AAAGGAGTTCATCTTTATTATGTTGGAGGGGAGGTGTATGCCGAATGCCTTAGTGACAG TAGCATCTTTGTGCAAAGTCGGAACTGCAACTACCATCATGGATTTCATCCTACTACTGT TTGCAAGATCCCTAGTGGGTGTAGTCTGAAAATTTTTAACAACCAAGAATTTGCTCAGTT ATTGGCACAGTCTGTGAACCATGGATTTGAGACAGTCTATGAGCTTACAAAAATGTGTA CTATACGTATGAGCTTTGTGAAGGGCTGGGGAGCAGAATACCACCGCCAGGATGTTAC TAGCACCCCCTGCTGGATTGAGATACATCTGCACGGCCCCCTCCAGTGGCTGGATAAA GTTCTTACTCAAATGGGTTCACCTCATAATCCTATTTCATCTGTATCTTAAATGGCCCCA GGCATCTGCCTCTGGAAAACTATTGAGCCTTGCATGTACTTGAAGGATGGATGAGTCA GACACGATTGAGAACTGACAAAGGAGCCTTGATAATACTTGACCTCTGTGACCAACTGT TGGATTCAGAAATTTAAACAAAAAAAAAAAAAAACACACACACCTTGGTAACATACTGTT GATATCAAGAACCTGTTTAGTTTACATTGTAACATTCTATTGTAAAATCAACTAAAATTCA GACTTTTAGCAGGACTTTGTGTACAGTTAAAGGAGAGATGGCCAAGCCAGGGACAAAT TGTCTATTAGAAAACGGTCCTAAGAGATTCTTTGGTGTTTGGCACTTTAAGGTCATCGTT GGGCAGAAGTTTAGCATTAATAGTTGTTCTGAAACGTGTTTTATCAGGTTTAGAGCCCA TGTTGAGTCTTCTTTTCATGGGTTTTCATAATATTTTAAAACTATTTGTTTAGCGATGGTT TTGTTCGTTTAAGTAAAGGTTAATCTTGATGATATACATAATAATCTTTCTAAAATTGTAT GCTGACCATACTTGCTGTCAGAATAATGCTAGGCATATGCTTTTTGCTAAATATGTATGT ACAGAGTATTTGGAAGTTAAGAATTGATTAGACTAGTGAATTTAGGAGTATTTGAGGTG GGTGGGGGGAAGAGGGAAATGACAACTGCAAATGTAGACTATACTGTAAAAATTCAGT TTGTTGCTTTAAAGAAACAAACTGATACCTGAATTTTGCTGTGTTTCCATTTTTTAGAGAT TTTTATCATTTTTTTCTCTCTCGGCATTCTTTTTTCTCATACTCTTCAAAAAGCAGTTCTG CAGCTGGTTAATTCATGTAACTGTGAGAGCAAATGAATAATTCCTGCTATTCTGAAATTG CCTACATGTTTCAATACCAGTTATATGGAGTGCTTGAATTTAATAAGCAGTTTTTACGGA GTTTACAGTACAGAAATAGGCTTTAATTTTCAAGTGAATTTTTTGCCAAACTTAGTAACTC TGTTAAATATTTGGAGGATTTAAAGAACATCCCAGTTTGAATTCATTTCAAACTTTTTAAA TTTTTTTGTACTATGTTTGGTTTTATTTTCCTTCTGTTAATCTTTTGTATTCACTTATGCTC TCGTACATTGAGTACTTTTATTCCAAAACTAGTGGGTTTTCTCTACTGGAAATTTTCAATA AACCTGTCATTATTGCTTACTTTGATTAAAA. (SEQ ID NO:260; NM_001003688.1), or a nucleic acid sequence that hybridizes to a nucleic acid sequence consisting of SEQ ID NO:260 under stringent hybridization conditions

In some embodiments, menin 1 (MEN1, MEAl, SCG2) comprises the amino acid sequence: MGLKAAQKTLFPLRSIDDVVRLFAAELGREEPDLVLLSLVLGFVEHFLAVNRVIPTNVPELTF QPSPAPDPPGGLTYFPVADLSIIAALYARFTAQIRGAVDLSLYPREGGVSSRELVKKVSDVI WNSLSRSYFKDRAHIQSLFSFITGWSPVGTKLDSSGVAFAVVGACQALGLRDVHLALSEDH AWVVFGPNGEQTAEVTWHGKGNEDRRGQTVNAGVAERSWLYLKGSYMRCDRKMEVAF MVCAINPSIDLHTDSLELLQLQQKLLWLLYDLGHLERYPMALGNLADLEELEPTPGRPDPLT LYHKGIASAKTYYRDEHIYPYMYLAGYHCRNRNVREALQAWADTATVIQDYNYCREDEEIY KEFFEVANDVIPNLLKEAASLLEAGEERPGEQSQGTQSQGSALQDPECFAHLLRFYDGICK WEEGSPTPVLHVGWATFLVQSLGRFEGQVRQKVRIVSREAEAAEAEEPWGEEAREGRRR GPRRESKPEEPPPPKKPALDKGLGTGQGAVSGPPRKPPGTVAGTARGPEGGSTAQVPAP AASPPPEGPVLTFQSEKMKGMKELLVATKINSSAIKLQLTAQSQVQMKKQKVSTPSDYTLS FLKRQRKGL. (SEQ ID NO:261; NP_000235.2NP_001180.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:261)

In some embodiments, the nucleic acid sequence encoding MEN1 comprises the nucleic acid sequence: GGTGTCCGGAGCCGCGGACCTAGAGATCCCAGAAGCCACAGCGCAGCGGCCCGGCC CGCCACTATTTCCAGGCTCAGCGGGGCAGGGGCCGCCGCCCACCGCCCGCCGCCAT GGGGCTGAAGGCCGCCCAGAAGACGCTGTTCCCGCTGCGCTCCATCGACGACGTGGT GCGCCTGTTTGCTGCCGAGCTGGGCCGAGAGGAGCCGGACCTGGTGCTCCTTTCCTT GGTGCTGGGCTTCGTGGAGCATTTTCTGGCTGTCAACCGCGTCATCCCTACCAACGTT CCCGAGCTCACCTTCCAGCCCAGCCCCGCCCCCGACCCGCCTGGCGGCCTCACCTAC TTTCCCGTGGCCGACCTGTCTATCATCGCCGCCCTCTATGCCCGCTTCACCGCCCAGA TCCGAGGCGCCGTCGACCTGTCCCTCTATCCTCGAGAAGGGGGTGTCTCCAGCCGTG AGCTGGTGAAGAAGGTCTCCGATGTCATATGGAACAGCCTCAGCCGCTCCTACTTCAA GGATCGGGCCCACATCCAGTCCCTCTTCAGCTTCATCACAGGTTGGAGCCCAGTAGGC ACCAAATTGGACAGCTCCGGTGTGGCCTTTGCTGTGGTTGGGGCCTGCCAGGCCCTG GGTCTCCGGGATGTCCACCTCGCCCTGTCTGAGGATCATGCCTGGGTAGTGTTTGGGC CCAATGGGGAGCAGACAGCTGAGGTCACCTGGCACGGCAAGGGCAACGAGGACCGC AGGGGCCAGACAGTCAATGCCGGTGTGGCTGAGCGGAGCTGGCTGTACCTGAAAGGA TCATACATGCGCTGTGACCGCAAGATGGAGGTGGCGTTCATGGTGTGTGCCATCAACC CTTCCATTGACCTGCACACCGACTCGCTGGAGCTTCTGCAGCTGCAGCAGAAGCTGCT CTGGCTGCTCTATGACCTGGGACATCTGGAAAGGTACCCCATGGCCTTAGGGAACCTG GCAGATCTAGAGGAGCTGGAGCCCACCCCTGGCCGGCCAGACCCACTCACCCTCTAC CACAAGGGCATTGCCTCAGCCAAGACCTACTATCGGGATGAACACATCTACCCCTACA TGTACCTGGCTGGCTACCACTGTCGCAACCGCAATGTGCGGGAAGCCCTGCAGGCCT GGGCGGACACGGCCACTGTCATCCAGGACTACAACTACTGCCGGGAAGACGAGGAGA TCTACAAGGAGTTCTTTGAAGTAGCCAATGATGTCATCCCCAACCTGCTGAAGGAGGCA GCCAGCTTGCTGGAGGCGGGCGAGGAGCGGCCGGGGGAGCAAAGCCAGGGCACCC AGAGCCAAGGTTCCGCCCTCCAGGACCCTGAGTGCTTCGCCCACCTGCTGCGATTCTA CGACGGCATCTGCAAATGGGAGGAGGGCAGTCCCACGCCTGTGCTGCACGTGGGCTG GGCCACCTTTCTTGTGCAGTCCCTAGGCCGTTTTGAGGGACAGGTGCGGCAGAAGGT GCGCATAGTGAGCCGAGAGGCCGAGGCGGCCGAGGCCGAGGAGCCGTGGGGCGAG GAAGCCCGGGAAGGCCGGCGGCGGGGCCCACGGCGGGAGTCCAAGCCAGAGGAGC CCCCGCCGCCCAAGAAGCCAGCACTGGACAAGGGCCTGGGCACCGGCCAGGGTGCA GTGTCAGGACCCCCCCGGAAGCCTCCTGGGACTGTCGCTGGCACAGCCCGAGGCCCT GAAGGTGGCAGCACGGCTCAGGTGCCAGCACCCGCAGCATCACCACCGCCGGAGGG TCCAGTGCTCACTTTCCAGAGTGAGAAGATGAAGGGCATGAAGGAGCTGCTGGTGGCC ACCAAGATCAACTCGAGCGCCATCAAGCTGCAACTCACGGCACAGTCGCAAGTGCAGA TGAAGAAGCAGAAAGTGTCCACCCCTAGTGACTACACTCTGTCTTTCCTCAAGCGGCA GCGCAAAGGCCTCTGAACTACTGGGGACTTCGGACCGCTTGTGGGGACCCAGGCTCC GCCCTTAGTCCCCCAACTCTGAGCCCATGTTCTGCCCCCAGCCCAAAGGGGACAGGC CTCACCTCTACCCAAACCCTAGGTTCCCGGTCCCGAGTACAGTCTGTATCAAACCCAC GATTTTCTCCAGCTCAGAACCCAGGGCTCTGCCCCAGTCGTTAGAATATAGGTCTCTTC TCCCAGAATCCCAGCCGGCCAATGGAAACCTCACGCTGGGTCCTAATTACCAGTCTTT AAAGGCCCAGCCCCTAGAAACCCAAGCTCCTCCTCGGAACCGCTCACCTAGAGCCAGA CCAACGTTACTCAGGGCTCCTCCCAGCTTGTAGGAGCTGAGGTTTCACCCTTAACCCA AGGAGCACAGGTCCCACCTCCAGCCCGGGAGCCTAGGACCACTCAGCCCCTAGGAGT ATATTTCCGCACTTCAGAATTCCATATCTTGCGAATCCAAGCTCCCTGCCCCAAATAACT TCAGTCCTGCTCCAGAATTTGGAAATCCTAGTTTCCTCTCCTTCGTATCCCGAGTCTGG GACACAAAACTCCGCCCCCAGCCTATGAGCATCCTGAGCCCCGCCCTCTTCCTGACGA AACTGGCCCCGGATCAGAGCAGGACCTCCCTTCCGACCCTCTGGGAACCTCCCAGAG GTCCAGCCCATCTCGGAGCATCCCGGAGGAAATCTGCAGAGGGTTAGGAGTGGGTGA CAAGAGCCTGATCTCTTCCTGTTTTGTACATAGATTTATTTTTCAGTTCCAAGAAAGATG AATACATTTTGTTAAAAAAAATAAAAAAAA. (SEQ ID NO:262; NM_000244.3), or a nucleic acid sequence that hybridizes to a nucleic acid sequence consisting of SEQ ID NO:262 under stringent hybridization conditions

In some embodiments, msh homeobox 1 (MSX1, HOX7, HYD1, ECTD3, SThaG1) comprises the amino acid sequence: MAPAADMTSLPLGVKVEDSAFGKPAGGGAGQAPSAAAATAAAMGADEEGAKPKVSPSLL PFSVEALMADHRKPGAKESALAPSEGVQAAGGSAQPLGVPPGSLGAPDAPSSPRPLGHF SVGGLLKLPEDALVKAESPEKPERTPWMQSPRFSPPPARRLSPPACTLRKHKTNRKPRTP FTTAQLLALERKFRQKQYLSIAERAEFSSSLSLTETQVKIWFQNRRAKAKRLQEAELEKLKM AAKPMLPPAAFGLSFPLGGPAAVAAAAGASLYGASGPFQRAALPVAPVGLYTAHVGYSMY HLT. (SEQ ID NO:263; NP_002439.2), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:263)

In some embodiments, the nucleic acid sequence encoding MSX comprises the nucleic acid sequence: AGGGCCCGGAGCCGGCGAGTGCTCCCGGGAACTCTGCCTGCGCGGCGGCAGCGACC GGAGGCCAGGCCCAGCACGCCGGAGCTGGCCTGCTGGGGAGGGGCGGGAGGCGCG CGCGGGAGGGTCCGCCCGGCCAGGGCCCCGGGCGCTCGCAGAGGCCGGCCGCGCT CCCAGCCCGCCCGGAGCCCATGCCCGGCGGCTGGCCAGTGCTGCGGCAGAAGGGGG GGCCCGGCTCTGCATGGCCCCGGCTGCTGACATGACTTCTTTGCCACTCGGTGTCAAA GTGGAGGACTCCGCCTTCGGCAAGCCGGCGGGGGGAGGCGCGGGCCAGGCCCCCA GCGCCGCCGCGGCCACGGCAGCCGCCATGGGCGCGGACGAGGAGGGGGCCAAGCC CAAAGTGTCCCCTTCGCTCCTGCCCTTCAGCGTGGAGGCGCTCATGGCCGACCACAG GAAGCCGGGGGCCAAGGAGAGCGCCCTGGCGCCCTCCGAGGGCGTGCAGGCGGCG GGTGGCTCGGCGCAGCCACTGGGCGTCCCGCCGGGGTCGCTGGGAGCCCCGGACG CGCCCTCTTCGCCGCGGCCGCTCGGCCATTTCTCGGTGGGGGGACTCCTCAAGCTGC CAGAAGATGCGCTCGTCAAAGCCGAGAGCCCCGAGAAGCCCGAGAGGACCCCGTGGA TGCAGAGCCCCCGCTTCTCCCCGCCGCCGGCCAGGCGGCTGAGCCCCCCAGCCTGC ACCCTCCGCAAACACAAGACGAACCGTAAGCCGCGGACGCCCTTCACCACCGCGCAG CTGCTGGCGCTGGAGCGCAAGTTCCGCCAGAAGCAGTACCTGTCCATCGCCGAGCGC GCGGAGTTCTCCAGCTCGCTCAGCCTCACTGAGACGCAGGTGAAGATATGGTTCCAGA ACCGCCGCGCCAAGGCAAAGAGACTACAAGAGGCAGAGCTGGAGAAGCTGAAGATGG CCGCCAAGCCCATGCTGCCACCGGCTGCCTTCGGCCTCTCCTTCCCTCTCGGCGGCC CCGCAGCTGTAGCGGCCGCGGCGGGTGCCTCGCTCTACGGTGCCTCTGGCCCCTTCC AGCGCGCCGCGCTGCCTGTGGCGCCCGTGGGACTCTACACGGCCCATGTGGGCTACA GCATGTACCACCTGACATAGAGGGTCCCAGGTCGCCCACCTGTGGGCCAGCCGATTC CTCCAGCCCTGGTGCTGTACCCCCGACGTGCTCCCCTGCTCGGCACCGCCAGCCGCC TTCCCTTTAACCCTCACACTGCTCCAGTTTCACCTCTTTGCTCCCTGAGTTCACTCTCCG AAGTCTGATCCCTGCCAAAAAGTGGCTGGAAGAGTCCCTTAGTACTCTTCTAGCATTTA GATCTACACTCTCGAGTTAAAGATGGGGAAACTGAGGGCAGAGAGGTTAACAGATTTAT CTAAGGTCCCCAGCAGAATTGACAGTTGAACAGAGCTAGAGGCCATGTCTCCTGCATA GCTTTTCCCTGTCCTGACACCAGGCAAGAAAAGCGCAGAGAAATCGGTGTCTGACGAT TTTGGAAATGAGAACAATCTCAAAAAAAAAAAAAAAAAAAAAAAAAAAAAAAAAAAAGAA AAGAGAAAAAAAAGACTAGCCAGCCAGGAAGATGAATCCTAGCTTCTTCCATTGGAAAA TTTAAGACAAGTTCAACAACAAAACATTTGCTCTGGGGGGCAGGGAAAACACAGATGTG TTGCAAAGGTAGGTTGAAGGGACCTCTCTCTTACCAGTACCAGAAACACAATTGTAAAA TTAAAAAAAAAAAAAAACTCTTTCTATTTAACAGTACATTTGTGTGGCTCTCAAACATCCC TTTGGAAGGGATTGTGTGTACTATGTAATATACTGTATATTTGAAATTTTATTATCATTTA TATTATAGCTATATTTGTTAAATAAATTAATTTTAAGCTACAAAAA. (SEQ ID NO:264; NM_002448.3), or a nucleic acid sequence that hybridizes to a nucleic acid sequence consisting of SEQ ID NO:264 under stringent hybridization conditions

In some embodiments, msh homeobox 2 (MSX2, FPP, MSH, PFM, CRS2, HOX8, PFM1) comprises the amino acid sequence: MASPSKGNDLFSPDEEGPAVVAGPGPGPGGAEGAAEERRVKVSSLPFSVEALMSDKKPP KEASPLPAESASAGATLRPLLLSGHGAREAHSPGPLVKPFETASVKSENSEDGAAWMQEP GRYSPPPSECAPGQE. (SEQ ID NO:265; NP_001350555.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:265)

In some embodiments, the nucleic acid sequence encoding MSX2 comprises the nucleic acid sequence: GCAGCAAAAAAGTTTGAGTCGCCGCTGCCGGGTTGCCAGCGGAGTCGCGCGTCGGGA GCTACGTAGGGCAGAGAAGTCATGGCTTCTCCGTCCAAAGGCAATGACTTGTTTTCGC CCGACGAGGAGGGCCCAGCAGTGGTGGCCGGACCAGGCCCGGGGCCTGGGGGCGC CGAGGGGGCCGCGGAGGAGCGCCGCGTCAAGGTCTCCAGCCTGCCCTTCAGCGTGG AGGCGCTCATGTCCGACAAGAAGCCGCCCAAGGAGGCGTCCCCGCTGCCGGCCGAAA GCGCCTCGGCCGGGGCCACCCTGCGGCCACTGCTGCTGTCGGGGCACGGCGCTCGG GAAGCGCACAGCCCCGGGCCGCTGGTGAAGCCCTTCGAGACCGCCTCGGTCAAGTCG GAAAATTCAGAAGATGGAGCGGCGTGGATGCAGGAACCCGGCCGATATTCGCCGCCG CCAAGTGAGTGCGCGCCGGGGCAGGAGTAGGAGGACATATGAGCCCTACCACCTGCA CCCTGAGGAAACACAAGACCAATCGGAAGCCGCGCACGCCCTTTACCACATCCCAGCT CCTCGCCCTGGAGCGCAAGTTCCGTCAGAAACAGTACCTCTCCATTGCAGAGCGTGCA GAGTTCTCCAGCTCTCTGAACCTCACAGAGACCCAGGTCAAAATCTGGTTCCAGAACC GAAGGGCCAAGGCGAAAAGACTGCAGGAGGCAGAACTGGAAAAGCTGAAAATGGCTG CAAAACCTATGCTGCCCTCCAGCTTCAGTCTCCCTTTCCCCATCAGCTCGCCCCTGCA GGCAGCGTCCATATATGGAGCATCCTACCCGTTCCATAGACCTGTGCTTCCCATCCCG CCTGTGGGACTCTATGCCACGCCAGTGGGATATGGCATGTACCACCTGTCCTAAGGAA GACCAGATCAATAGACTCCATGATGGATGCTTGTTTCAAAGGGTTTCCTCTCCCTCTCC ACGAAGGCAGTACCAGCCAGTACTCCTGCTCTGCTAACCCTGCGTGCACCACCCTAAG CGGCTAGGCTGACAGGGCCACACGACATAGCTGAAATTTGTTCTGTAGGCGGAGGCA CCAAGCCCTGTTTTCTTGGTGTAATCTTCCAGATGCCCCCTTTTCCTTTCACAAAGATTG GCTCTGATGGTTTTTATGTATAAATATATATATATAATAAAATATAATACATTTTTATACAG CAGACGTAAAAATTCAAATTATTTTAAAAGGCAAAATTTATATACATATGTGCTTTTTTTC TATATCTCACCTTCCCAAAAGACACTGTGTAAGTCCATTTGTTGTATTTTCTTAAAGAGG GAGACAAATTATTTGCAAAATGTGCTAAAGTCAATGATTTTTACGGGATTATTGACTTCT GCTTATGGAAAACAAAGAAACAGACACAATGCACACAGAAAATATTAGATATGGAGAGA TTATTCAAAGTGAAGGGGACACATCATATTTCTGCATTTTACTTGCATTAAAAGAAACCT CTTTATATACTACAGTTGTTCCTATCTCTCCCCCGCCCCCCACCGCCCCACCACACACA TATTTTTAAAGTTTTTCCTTTTTTAAGAATATTTTTGTAAGACCAATACCTGGGATGAGAA GAATCCTGAGACTGCCTGGAGGTGAGGTAGAAAATTAGAAATACTTCCTAATTCTTCTC AAGGCTGTTGGTAACTTTATTTCAGATAATTGGAGAGTAAAATGTTAAAACCTGTTGAGA GGAATTGATGGTTTCTGAGAAATACTAGGTACATTCATCCTCACAGATTGCAAAGGTGA TTTGGGTGGGGGTTTAGTAATTTTCTGCTTAAAAAATGAGTATCTTGTAACCATTACCTA TATGCTAAATATTCTTGAACAATTAGTAGATCCAGAAAGAAAAAAAAATATGCTTTCTCT GTGTGTGTACCTGTTGTATGTCCTAAACTTATTAGAAAATTTTATATACTTTTTTACATGT TGGGGGGCAGAAGGTAAAGCCATGTTTTGACTTGGTGAAAATGGGATTGTCAAACAGC CCATTAAGTTCCCTGGTATTTCACCTTCCTGTCCATCTGTCCCCTCCCTCCGGTATACC TTTATCCCTTTGAAAGGGTGCTTGTACAATTTGATATATTTTATTGAAGAGTTATCTCTTA TTCTGAATTAAATTAAGCATTTGTTTTATTGCAGTAAAGTTTGTCCAAACTCACAA (SEQ ID NO:266; NM_001363626.2), or a nucleic acid sequence that hybridizes to a nucleic acid sequence consisting of SEQ ID NO:266 under stringent hybridization conditions

In some embodiments, neurofibromin 1 (NF1, WSS, NFNS, VRNF) comprises the amino acid sequence: MAAHRPVEWVQAVVSRFDEQLPIKTGQQNTHTKVSTEHNKECLINISKYKFSLVISGLTTILK NVNNMRIFGEAAEKNLYLSQLIILDTLEKCLAGQPKDTMRLDETMLVKQLLPEICHFLHTCRE GNQHAAELRNSASGVLFSLSCNNFNAVFSRISTRLQELTVCSEDNVDVHDIELLQYINVDCA KLKRLLKETAFKFKALKKVAQLAVINSLEKAFWNWVENYPDEFTKLYQIPQTDMAECAEKLF DLVDGFAESTKRKAAVWPLQIILLILCPEIIQDISKDVVDENNMNKKLFLDSLRKALAGHGGS RQLTESAAIACVKLCKASTYINWEDNSVIFLLVQSMVVDLKNLLFNPSKPFSRGSQPADVDL MIDCLVSCFRISPHNNQHFKICLAQNSPSTFHYVLVNSLHRIITNSALDWWPKIDAVYCHSVE LRNMFGETLHKAVQGCGAHPAIRMAPSLTFKEKVTSLKFKEKPTDLETRSYKYLLLSMVKLI HADPKLLLCNPRKQGPETQGSTAELITGLVQLVPQSHMPEIAQEAMEALLVLHQLDSIDLW NPDAPVETFWEISSQMLFYICKKLTSHQMLSSTEILKWLREILICRNKFLLKNKQADRSSCHF LLFYGVGCDIPSSGNTSQMSMDHEELLRTPGASLRKGKGNSSMDSAAGCSGTPPICRQAQ TKLEVALYMFLWNPDTEAVLVAMSCFRHLCEEADIRCGVDEVSVHNLLPNYNTFMEFASVS NMMSTGRAALQKRVMALLRRIEHPTAGNTEAWEDTHAKWEQATKLILNYPKAKMEDGQAA ESLHKTIVKRRMSHVSGGGSIDLSDTDSLQEWINMTGFLCALGGVCLQQRSNSGLATYSPP MGPVSERKGSMISVMSSEGNADTPVSKFMDRLLSLMVCNHEKVGLQIRTNVKDLVGLELS PALYPMLFNKLKNTISKFFDSQGQVLLTDTNTQFVEQTIAIMKNLLDNHTEGSSEHLGQASIE TMMLNLVRYVRVLGNMVHAIQIKTKLCQLVEVMMARRDDLSFCQEMKFRNKMVEYLTDWV MGTSNQAADDDVKCLTRDLDQASMEAVVSLLAGLPLQPEEGDGVELMEAKSQLFLKYFTL FMNLLNDCSEVEDESAQTGGRKRGMSRRLASLRHCTVLAMSNLLNANVDSGLMHSIGLGY HKDLQTRATFMEVLTKILQQGTEFDTLAETVLADRFERLVELVTMMGDQGELPIAMALANV VPCSQWDELARVLVTLFDSRHLLYQLLWNMFSKEVELADSMQTLFRGNSLASKIMTFCFKV YGATYLQKLLDPLLRIVITSSDWQHVSFEVDPTRLEPSESLEENQRNLLQMTEKFFHAIISSS SEFPPQLRSVCHCLYQVVSQRFPQNSIGAVGSAMFLRFINPAIVSPYEAGILDKKPPPRIER GLKLMSKILQSIANHVLFTKEEHMRPFNDFVKSNFDAARRFFLDIASDCPTSDAVNHSLSFIS DGNVLALHRLLWNNQEKIGQYLSSNRDHKAVGRRPFDKMATLLAYLGPPEHKPVADTHWS SLNLTSSKFEEFMTRHQVHEKEEFKALKTLSIFYQAGTSKAGNPIFYYVARRFKTGQINGDL LIYHVLLTLKPYYAKPYEIVVDLTHTGPSNRFKTDFLSKWFVVFPGFAYDNVSAVYIYNCNS WVREYTKYHERLLTGLKGSKRLVFIDCPGKLAEHIEHEQQKLPAATLALEEDLKVFHNALKL AHKDTKVSIKVGSTAVQVTSAERTKVLGQSVFLNDIYYASEIEEICLVDENQFTLTIANQGTP LTFMHQECEAIVQSIIHIRTRWELSQPDSIPQHTKIRPKDVPGTLLNIALLNLGSSDPSLRSAA YNLLCALTCTFNLKIEGQLLETSGLCIPANNTLFIVSISKTLAANEPHLTLEFLEECISGFSKSSI ELKHLCLEYMTPWLSNLVRFCKHNDDAKRQRVTAILDKLITMTINEKQMYPSIQAKIWGSLG QITDLLDVVLDSFIKTSATGGLGSIKAEVMADTAVALASGNVKLVSSKVIGRMCKIIDKTCLSP TPTLEQHLMWDDIAILARYMLMLSFNNSLDVAAHLPYLFHVVTFLVATGPLSLRASTHGLVIN IIHSLCTCSQLHFSEETKQVLRLSLTEFSLPKFYLLFGISKVKSAAVIAFRSSYRDRSFSPGSY ERETFALTSLETVTEALLEIMEACMRDIPTCKWLDQWTELAQRFAFQYNPSLQPRALVVFG CISKRVSHGQIKQIIRILSKALESCLKGPDTYNSQVLIEATVIALTKLQPLLNKDSPLHKALFWV AVAVLQLDEVNLYSAGTALLEQNLHTLDSLRIFNDKSPEEVFMAIRNPLEWHCKQMDHFVG LNFNSNFNFALVGHLLKGYRHPSPAIVARTVRILHTLLTLVNKHRNCDKFEVNTQSVAYLAA LLTVSEEVRSRCSLKHRKSLLLTDISMENVPMDTYPIHHGDPSYRTLKETQPWSSPKGSEG YLAATYPTVGQTSPRARKSMSLDMGQPSQANTKKLLGTRKSFDHLISDTKAPKRQEMESGI TTPPKMRRVAETDYEMETQRISSSQQHPHLRKVSVSESNVLLDEEVLTDPKIQALLLTVLAT LVKYTTDEFDQRILYEYLAEASVVFPKVFPVVHNLLDSKINTLLSLCQDPNLLNPIHGIVQSVV YH EESPPQYQTSYLQSFGFNGLWRFAGPFSKQTQIPDYAELIVKFLDALIDTYLPGIDEETS EESLLTPTSPYPPALQSQLSITANLNLSNSMTSLATSQHSPGIDKENVELSPTTGHCNSGRT RHGSASQVQKQRSAGSFKRNSIKKIV. (SEQ ID NO:267; NP_000258.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:267)

In some embodiments, the nucleic acid sequence encoding NF1 comprises the nucleic acid sequence: AATCTCTAGCTCGCTCGCGCTCCCTCTCCCCGGGCCGTGGAAAGGATCCCACTTCCGG TGGGGTGTCATGGCGGCGTCTCGGACTGTGATGGCTGTGGGGAGACGGCGCTAGTG GGGAGAGCGACCAAGAGGCCCCCTCCCCTCCCCGGGTCCCCTTCCCCTATCCCCCTC CCCCCAGCCTCCTTGCCAACGCCCCCTTTCCCTCTCCCCCTCCCGCTCGGCGCTGACC CCCCATCCCCACCCCCGTGGGAACACTGGGAGCCTGCACTCCACAGACCCTCTCCTT GCCTCTTCCCTCACCTCAGCCTCCGCTCCCCGCCCTCTTCCCGGCCCAGGGCGCCGG CCCACCCTTCCCTCCGCCGCCCCCCGGCCGCGGGGAGGACATGGCCGCGCACAGGC CGGTGGAATGGGTCCAGGCCGTGGTCAGCCGCTTCGACGAGCAGCTTCCAATAAAAA CAGGACAGCAGAACACACATACCAAAGTCAGTACTGAGCACAACAAGGAATGTCTAAT CAATATTTCCAAATACAAGTTTTCTTTGGTTATAAGCGGCCTCACTACTATTTTAAAGAAT GTTAACAATATGAGAATATTTGGAGAAGCTGCTGAAAAAAATTTATATCTCTCTCAGTTG ATTATATTGGATACACTGGAAAAATGTCTTGCTGGGCAACCAAAGGACACAATGAGATT AGATGAAACGATGCTGGTCAAACAGTTGCTGCCAGAAATCTGCCATTTTCTTCACACCT GTCGTGAAGGAAACCAGCATGCAGCTGAACTTCGGAATTCTGCCTCTGGGGTTTTATTT TCTCTCAGCTGCAACAACTTCAATGCAGTCTTTAGTCGCATTTCTACCAGGTTACAGGA ATTAACTGTTTGTTCAGAAGACAATGTTGATGTTCATGATATAGAATTGTTACAGTATATC AATGTGGATTGTGCAAAATTAAAACGACTCCTGAAGGAAACAGCATTTAAATTTAAAGCC CTAAAGAAGGTTGCGCAGTTAGCAGTTATAAATAGCCTGGAAAAGGCATTTTGGAACTG GGTAGAAAATTATCCAGATGAATTTACAAAACTGTACCAGATCCCACAGACTGATATGG CTGAATGTGCAGAAAAGCTATTTGACTTGGTGGATGGTTTTGCTGAAAGCACCAAACGT AAAGCAGCAGTTTGGCCACTACAAATCATTCTCCTTATCTTGTGTCCAGAAATAATCCAG GATATATCCAAAGACGTGGTTGATGAAAACAACATGAATAAGAAGTTATTTCTGGACAG TCTACGAAAAGCTCTTGCTGGCCATGGAGGAAGTAGGCAGCTGACAGAAAGTGCTGCA ATTGCCTGTGTCAAACTGTGTAAAGCAAGTACTTACATCAATTGGGAAGATAACTCTGT CATTTTCCTACTTGTTCAGTCCATGGTGGTTGATCTTAAGAACCTGCTTTTTAATCCAAG TAAGCCATTCTCAAGAGGCAGTCAGCCTGCAGATGTGGATCTAATGATTGACTGCCTTG TTTCTTGCTTTCGTATAAGCCCTCACAACAACCAACACTTTAAGATCTGCCTGGCTCAGA ATTCACCTTCTACATTTCACTATGTGCTGGTAAATTCACTCCATCGAATCATCACCAATT CCGCATTGGATTGGTGGCCTAAGATTGATGCTGTGTATTGTCACTCGGTTGAACTTCGA AATATGTTTGGTGAAACACTTCATAAAGCAGTGCAAGGTTGTGGAGCACACCCAGCAAT ACGAATGGCACCGAGTCTTACATTTAAAGAAAAAGTAACAAGCCTTAAATTTAAAGAAAA ACCTACAGACCTGGAGACAAGAAGCTATAAGTATCTTCTCTTGTCCATGGTGAAACTAA TTCATGCAGATCCAAAGCTCTTGCTTTGTAATCCAAGAAAACAGGGGCCCGAAACCCAA GGCAGTACAGCAGAATTAATTACAGGGCTCGTCCAACTGGTCCCTCAGTCACACATGC CAGAGATTGCTCAGGAAGCAATGGAGGCTCTGCTGGTTCTTCATCAGTTAGATAGCATT GATTTGTGGAATCCTGATGCTCCTGTAGAAACATTTTGGGAGATTAGCTCACAAATGCT TTTTTACATCTGCAAGAAATTAACTAGTCATCAAATGCTTAGTAGCACAGAAATTCTCAA GTGGTTGCGGGAAATATTGATCTGCAGGAATAAATTTCTTCTTAAAAATAAGCAGGCAG ATAGAAGTTCCTGTCACTTTCTCCTTTTTTACGGGGTAGGATGTGATATTCCTTCTAGTG GAAATACCAGTCAAATGTCCATGGATCATGAAGAATTACTACGTACTCCTGGAGCCTCT CTCCGGAAGGGAAAAGGGAACTCCTCTATGGATAGTGCAGCAGGATGCAGCGGAACC CCCCCGATTTGCCGACAAGCCCAGACCAAACTAGAAGTGGCCCTGTACATGTTTCTGT GGAACCCTGACACTGAAGCTGTTCTGGTTGCCATGTCCTGTTTCCGCCACCTCTGTGA GGAAGCAGATATCCGGTGTGGGGTGGATGAAGTGTCAGTGCATAACCTCTTGCCCAAC TATAACACATTCATGGAGTTTGCCTCTGTCAGCAATATGATGTCAACAGGAAGAGCAGC ACTTCAGAAAAGAGTGATGGCACTGCTGAGGCGCATTGAGCATCCCACTGCAGGAAAC ACTGAGGCTTGGGAAGATACACATGCAAAATGGGAACAAGCAACAAAGCTAATCCTTAA CTATCCAAAAGCCAAAATGGAAGATGGCCAGGCTGCTGAAAGCCTTCACAAGACCATT GTTAAGAGGCGAATGTCCCATGTGAGTGGAGGAGGATCCATAGATTTGTCTGACACAG ACTCCCTACAGGAATGGATCAACATGACTGGCTTCCTTTGTGCCCTTGGGGGAGTGTG CCTCCAGCAGAGAAGCAATTCTGGCCTGGCAACCTATAGCCCACCCATGGGTCCAGTC AGTGAACGTAAGGGTTCTATGATTTCAGTGATGTCTTCAGAGGGAAACGCAGATACACC TGTCAGCAAATTTATGGATCGGCTGTTGTCCTTAATGGTGTGTAACCATGAGAAAGTGG GACTTCAAATACGGACCAATGTTAAGGATCTGGTGGGTCTAGAATTGAGTCCTGCTCTG TATCCAATGCTATTTAACAAATTGAAGAATACCATCAGCAAGTTTTTTGACTCCCAAGGA CAGGTTTTATTGACTGATACCAATACTCAATTTGTAGAACAAACCATAGCTATAATGAAG AACTTGCTAGATAATCATACTGAAGGCAGCTCTGAACATCTAGGGCAAGCTAGCATTGA AACAATGATGTTAAATCTGGCAGGTATGTTCGTGTGCTTGGGAATATGGTCCATGCAAT TCAAATAAAAACGAAACTGTGTCAATTAGTTGAAGTAATGATGGCAAGGAGAGATGACC TCTCATTTTGCCAAGAGATGAAATTTAGGAATAAGATGGTAGAATACCTGACAGACTGG GTTATGGGAACATCAAACCAAGCAGCAGATGATGATGTAAAATGTCTTACAAGAGATTT GGACCAGGCAAGCATGGAAGCAGTAGTTTCACTTCTAGCTGGTCTCCCTCTGCAGCCT GAAGAAGGAGATGGTGTGGAATTGATGGAAGCCAAATCACAGTTATTTCTTAAATACTT CACATTATTTATGAACCTTTTGAATGACTGCAGTGAAGTTGAAGATGAAAGTGCGCAAA CAGGTGGCAGGAAACGTGGCATGTCTCGGAGGCTGGCATCACTGAGGCACTGTACGG TCCTTGCAATGTCAAACTTACTCAATGCCAACGTAGACAGTGGTCTCATGCACTCCATA GGCTTAGGTTACCACAAGGATCTCCAGACAAGAGCTACATTTATGGAAGTTCTGACAAA AATCCTTCAACAAGGCACAGAATTTGACACACTTGCAGAAACAGTATTGGCTGATCGGT TTGAGAGATTGGTGGAACTGGTCACAATGATGGGTGATCAAGGAGAACTCCCTATAGC GATGGCTCTGGCCAATGTGGTTCCTTGTTCTCAGTGGGATGAACTAGCTCGAGTTCTG GTTACTCTGTTTGATTCTCGGCATTTACTCTACCAACTGCTCTGGAACATGTTTTCTAAA GAAGTAGAATTGGCAGACTCCATGCAGACTCTCTTCCGAGGCAACAGCTTGGCCAGTA AAATAATGACATTCTGTTTCAAGGTATATGGTGCTACCTATCTACAAAAACTCCTGGATC CTTTATTACGAATTGTGATCACATCCTCTGATTGGCAACATGTTAGCTTTGAAGTGGATC CTACCAGGTTAGAACCATCAGAGAGCCTTGAGGAAAACCAGCGGAACCTCCTTCAGAT GACTGAAAAGTTCTTCCATGCCATCATCAGTTCCTCCTCAGAATTCCCCCCTCAACTTC GAAGTGTGTGCCACTGTTTATACCAGGTGGTTAGCCAGCGTTTCCCTCAGAACAGCAT CGGTGCAGTAGGAAGTGCCATGTTCCTCAGATTTATCAATCCTGCCATTGTCTCACCGT ATGAAGCAGGGATTTTAGATAAAAAGCCACCACCTAGAATCGAAAGGGGCTTGAAGTTA ATGTCAAAGATACTTCAGAGTATTGCCAATCATGTTCTCTTCACAAAAGAAGAACATATG CGGCCTTTCAATGATTTTGTGAAAAGCAACTTTGATGCAGCACGCAGGTTTTTCCTTGA TATAGCATCTGATTGTCCTACAAGTGATGCAGTAAATCATAGTCTTTCCTTCATAAGTGA CGGCAATGTGCTTGCTTTACATCGTCTACTCTGGAACAATCAGGAGAAAATTGGGCAGT ATCTTTCCAGCAACAGGGATCATAAAGCTGTTGGAAGACGACCTTTTGATAAGATGGCA ACACTTCTTGCATACCTGGGTCCTCCAGAGCACAAACCTGTGGCAGATACACACTGGT CCAGCCTTAACCTTACCAGTTCAAAGTTTGAGGAATTTATGACTAGGCATCAGGTACAT GAAAAAGAAGAATTCAAGGCTTTGAAAACGTTAAGTATTTTCTACCAAGCTGGGACTTC CAAAGCTGGGAATCCTATTTTTTATTATGTTGCACGGAGGTTCAAAACTGGTCAAATCAA TGGTGATTTGCTGATATACCATGTCTTACTGACTTTAAAGCCATATTATGCAAAGCCATA TGAAATTGTAGTGGACCTTACCCATACCGGGCCTAGCAATCGCTTTAAAACAGACTTTC TCTCTAAGTGGTTTGTTGTTTTTCCTGGCTTTGCTTACGACAACGTCTCCGCAGTCTATA TCTATAACTGTAACTCCTGGGTCAGGGAGTACACCAAGTATCATGAGCGGCTGCTGAC TGGCCTCAAAGGTAGCAAAAGGCTTGTTTTCATAGACTGTCCTGGGAAACTGGCTGAG CACATAGAGCATGAACAACAGAAACTACCTGCTGCCACCTTGGCTTTAGAAGAGGACC TGAAGGTATTCCACAATGCTCTCAAGCTAGCTCACAAAGACACCAAAGTTTCTATTAAA GTTGGTTCTACTGCTGTCCAAGTAACTTCAGCAGAGCGAACAAAAGTCCTAGGGCAATC AGTCTTTCTAAATGACATTTATTATGCTTCGGAAATTGAAGAAATCTGCCTAGTAGATGA GAACCAGTTCACCTTAACCATTGCAAACCAGGGCACGCCGCTCACCTTCATGCACCAG GAGTGTGAAGCCATTGTCCAGTCTATCATTCATATCCGGACCCGCTGGGAACTGTCAC AGCCCGACTCTATCCCCCAACACACCAAGATTCGGCCAAAAGATGTCCCTGGGACACT GCTCAATATCGCATTACTTAATTTAGGCAGTTCTGACCCGAGTTTACGGTCAGCTGCCT ATAATCTTCTGTGTGCCTTAACTTGTACCTTTAATTTAAAAATCGAGGGCCAGTTACTAG AGACATCAGGTTTATGTATCCCTGCCAACAACACCCTCTTTATTGTCTCTATTAGTAAGA CACTGGCAGCCAATGAGCCACACCTCACGTTAGAATTTTTGGAAGAGTGTATTTCTGGA TTTAGCAAATCTAGTATTGAATTGAAACACCTTTGTTTGGAATACATGACTCCATGGCTG TCAAATCTAGTTCGTTTTTGCAAGCATAATGATGATGCCAAACGACAAAGAGTTACTGCT ATTCTTGACAAGCTGATAACAATGACCATCAATGAAAAACAGATGTACCCATCTATTCAA GCAAAAATATGGGGAAGCCTTGGGCAGATTACAGATCTGCTTGATGTTGTACTAGACAG TTTCATCAAAACCAGTGCAACAGGTGGCTTGGGATCAATAAAAGCTGAGGTGATGGCA GATACTGCTGTAGCTTTGGCTTCTGGAAATGTGAAATTGGTTTCAAGCAAGGTTATTGG AAGGATGTGCAAAATAATTGACAAGACATGCTTATCTCCAACTCCTACTTTAGAACAACA TCTTATGTGGGATGATATTGCTATTTTAGCACGCTACATGCTGATGCTGTCCTTCAACAA TTCCCTTGATGTGGCAGCTCATCTTCCCTACCTCTTCCACGTTGTTACTTTCTTAGTAGC CACAGGTCCGCTCTCCCTTAGAGCTTCCACACATGGACTGGTCATTAATATCATTCACT CTCTGTGTACTTGTTCACAGCTTCATTTTAGTGAAGAGACCAAGCAAGTTTTGAGACTCA GTCTGACAGAGTTCTCATTACCCAAATTTTACTTGCTGTTTGGCATTAGCAAAGTCAAGT CAGCTGCTGTCATTGCCTTCCGTTCCAGTTACCGGGACAGGTCATTCTCTCCTGGCTC CTATGAGAGAGAGACTTTTGCTTTGACATCCTTGGAAACAGTCACAGAAGCTTTGTTGG AGATCATGGAGGCATGCATGAGAGATATTCCAACGTGCAAGTGGCTGGACCAGTGGAC AGAACTAGCTCAAAGATTTGCATTCCAATATAATCCATCCCTGCAACCAAGAGCTCTTGT TGTCTTTGGGTGTATTAGCAAACGAGTGTCTCATGGGCAGATAAAGCAGATAATCCGTA TTCTTAGCAAGGCACTTGAGAGTTGCTTAAAAGGACCTGACACTTACAACAGTCAAGTT CTGATAGAAGCTACAGTAATAGCACTAACCAAATTACAGCCACTTCTTAATAAGGACTC GCCTCTGCACAAAGCCCTCTTTTGGGTAGCTGTGGCTGTGCTGCAGCTTGATGAGGTC AACTTGTATTCAGCAGGTACCGCACTTCTTGAACAAAACCTGCATACTTTAGATAGTCTC CGTATATTCAATGACAAGAGTCCAGAGGAAGTATTTATGGCAATCCGGAATCCTCTGGA GTGGCACTGCAAGCAAATGGATCATTTTGTTGGACTCAATTTCAACTCTAACTTTAACTT TGCATTGGTTGGACACCTTTTAAAAGGGTACAGGCATCCTTCACCTGCTATTGTTGCAA GAACAGTCAGAATTTTACATACACTACTAACTCTGGTTAACAAACACAGAAATTGTGACA AATTTGAAGTGAATACACAGAGCGTGGCCTACTTAGCAGCTTTACTTACAGTGTCTGAA GAAGTTCGAAGTCGCTGCAGCCTAAAACATAGAAAGTCACTTCTTCTTACTGATATTTCA ATGGAAAATGTTCCTATGGATACATATCCCATTCATCATGGTGACCCTTCCTATAGGACA CTAAAGGAGACTCAGCCATGGTCCTCTCCCAAAGGTTCTGAAGGATACCTTGCAGCCA CCTATCCAACTGTCGGCCAGACCAGTCCCCGAGCCAGGAAATCCATGAGCCTGGACAT GGGGCAACCTTCTCAGGCCAACACTAAGAAGTTGCTTGGAACAAGGAAAAGTTTTGAT CACTTGATATCAGACACAAAGGCTCCTAAAAGGCAAGAAATGGAATCAGGGATCACAAC ACCCCCCAAAATGAGGAGAGTAGCAGAAACTGATTATGAAATGGAAACTCAGAGGATTT CCTCATCACAACAGCACCCACATTTACGTAAAGTTTCAGTGTCTGAATCAAATGTTCTCT TGGATGAAGAAGTACTTACTGATCCGAAGATCCAGGCGCTGCTTCTTACTGTTCTAGCT ACACTGGTAAAATATACCACAGATGAGTTTGATCAACGAATTCTTTATGAATACTTAGCA GAGGCCAGTGTTGTGTTTCCCAAAGTCTTTCCTGTTGTGCATAATTTGTTGGACTCTAA GATCAACACCCTGTTATCATTGTGCCAAGATCCAAATTTGTTAAATCCAATCCATGGAAT TGTGCAGAGTGTGGTGTACCATGAAGAATCCCCACCACAATACCAAACATCTTACCTGC AAAGTTTTGGTTTTAATGGCTTGTGGCGGTTTGCAGGACCGTTTTCAAAGCAAACACAA ATTCCAGACTATGCTGAGCTTATTGTTAAGTTTCTTGATGCCTTGATTGACACGTACCTG CCTGGAATTGATGAAGAAACCAGTGAAGAATCCCTCCTGACTCCCACATCTCCTTACCC TCCTGCACTGCAGAGCCAGCTTAGTATCACTGCCAACCTTAACCTTTCTAATTCCATGA CCTCACTTGCAACTTCCCAGCATTCCCCAGGAATCGACAAGGAGAACGTTGAACTCTC CCCTACCACTGGCCACTGTAACAGTGGACGAACTCGCCACGGATCCGCAAGCCAAGT GCAGAAGCAAAGAAGCGCTGGCAGTTTCAAACGTAATAGCATTAAGAAGATCGTGTGA AGCTTGCTTGCTTTCTTTTTTAAAATCAACTTAACATGGGCTCTTCACTAGTGACCCCTT CCCTGTCCTTGCCCTTTCCCCCCATGTTGTAATGCTGCACTTCCTGTTTTATAATGAACC CATCCGGTTTGCCATGTTGCCAGATGATCAACTCTTCGAAGCCTTGCCTAAATTTAATG CTGCCTTTTCTTTAACTTTTTTTCTTCTACTTTTGGCGTGTATCTGGTATATGTAAGTGTT CAGAACAACTGCAAAGAAAGTGGGAGGTCAGGAAACTTTTAACTGAGAAATCTCAATTG TAAGAGAGGATGAATTCTTGAATACTGCTACTACTGGCCAGTGATGAAAGCCATTTGCA CAGAGCTCTGCCTTCTGTGGTTTTCCCTTCTTCATCCTACAGAGTAAAGTGTTAGTCCTA TTTATACATTTTTCAAGATACAAGTTTATGAGAGAAATAGTATTATAACCCCAGTATGTTT AATCTTTTAGCTGTGGACTTTTTTTTTAACCGTACAAAACTGAAAGAACCATAGAGGTCA AGCCTCAGTGACTTGACACCATAAAGCCACAGACAAGGTACTTGGGGGGGAGGGCAG GGAAATTTCATATTTTATAGTGGATTCTTAAGAAATACTAACACTTGAGTATTAGCAATAA TTACAGGAAAATAAGTGCGACCACATATATCTTAACATTACTGAATTAAAACTATGGCTT CTAAGTCCTTATCCAAACTCAGTCATCCAAACTAGTTTATTTTTTTCTCCAGTTGATTATC TTTTAATTTTTAATTTTGCTAAAGGTGGTTTTTTTGTGTTTTGTTTTTTGTAAACCAAAACT ATACTAAGTATAGTAATTATATATATATATATATTTTTTCCCCTCCCCCTCTTCTTTCCTAA CTAATTCTGAGCAGGGTAATCAGTGAACAAAGTGTTGAAAATTGTTCCCAGAAGGTAAT TTTCATAGATGTTTGCATTAGCTCCATAGCAAAATGGAATGGTACGTGACATTTAGGGTA GCTGATATTTTTATTTTGTTAAATAATTTCCAAGAATAGAGTATGGTGTATATTATAAATTT CTTTGATAAGATGTATTTTGAATGTCTTTTAATCTTCCTCCTCCTCTCCAAAAAAATCAGA AACCTCTTTAAGAAAACATGTAGGTTATATATGCTAGAATTGCATTTAATCACTGTGAAA AGACTGGTCAGCCTGCATTAGTATGACAGTAGGGGGGCTGTTAGAATTGCTGCTATAC TGGTGGTATGGATTATCATGGCATTGGAATTTTCATAGTAATGCAGATCCAATTTCTTTG TGGTACCTGCAGTTTACAAAATAATTTGACTTCAGTGAGCATATTGGTATCTGGATGTTC CAATTTAGAACTAAACCATATTTATTACAAAAAGATATTAATCCCTCTACTCCCAGGTTCC CTTTATATGTTAAGATATAATGGCTTTGAGGGGGGAAAAAATAAACCTAGGGGAGAGGG GAGTTTCCTGTAGTGCTGTTTCATTAGAGGATTTCAGTAAATTAAATTCCACAGCTAATT CAATAAATAATGGTACATTTAAGTGTTCTGATTTTAATAATATATTTCACATTTATCCACA CAGTAACAATGTAATATGTTAATGTAAATAAAATTGGTTTTGATACTCAGAAATAACAAGA ATTTAATTTTTTAAATTTGTTTACAGTCCTGGGAAAAGTAAGAATTATTTGCCAAAATAAG AGGAAAGAAAACCTTAGTATTATTAATGAGTTTACCATAGAATTGTTGGAAATACTGAAG ACAGGTGCAATTTACTAAACTTTTGTTTTTAAACTATTGTAGAGGCTGCATTAGAAGAAA ATGTTTATAATGACAGAGCAACTATGACTATATAAAAAAGCTGAAATTAGAACTGTGTTT AGAAATAGATCAGTAACCCAGTGCCAAGGATGCCAAGCTGCCACCATGGTCTTGGCTC TCCCACAACCCAGTGTTTCTGGGGTAAGTTTCACAGTTTCTAGGCCCTGGAATAGCAG GCAGTGTAAGCCTTTGATAACTTTAGTTCGATGTTTTTCTTGTTTTTGTTTGTTGGTTTGG TGCATATGATAGTGGGTGTTATGCTATTTTGCTCTTCCCATCAAAATAAAGAAACTTCCA GAGGTTTACTGTTAAAAATACTGATATTTCCATAAACGGGTTTACCAAGGGTGTAGTATT TCATACCGCCTGAAATGATCAGCATTGGCACAAATCAAAATTCAGCCGCCTTTGAAATG CAAAAATACCTTTGACTAGTAAGTACATCCTAGGAGTTTGAAAACTTAACTAAGGTTTAA AATTTACCTTGTTTAAAGAACTTCTGACTTTTGAGGAAAATCTAGCTTTCCAAGTAACTAA AATGTACATGAGATAAACCTCTCACCACTATGTGTCCCTTGAGAAATGCAACACTTTTTT AGTCTTCATACTTGTAATCTATAAAAGAAATTCTGAAGTTTAGACCAAGTTGCCCATTTC TGCGTAATTGACATAAGTTCTGTTAAAAATATTATAAGTAATTCGTTTCGGTTTGTAGATG TTTCCCCTGACTTGTTAAAGAGGAAACCAGGAACTCAGTCATGTTTTTGTCCTGGATAAT CTACCTGTTATGCCAGTACTCCCATCCGAGGGGCATGCCCTTAGTTGCCCAGATGGAG ATGCAGTTCAGTAGATTTGGGGCAAAGTGGCTACAGCTCTGTCTTCCATTCACTCAACA CCTGTTCATGACTGAGCCAGGTGCCCAGGACACATCCTAAACAGTCAGCTTCTATCCT GTGTCCTAGTTGGGGAGACAGAGTGCCAGCCAGCAACCCTCCCAGGTTTGTAGGTTTT AGGGGTTTTCAGTTTTGTTTGGGTTTTTTGTTTTTTGTTTTTGTTTCTACATCCTTCCCCG ACTCCCAGGCATAATGAGGCATGTCTTACTCAATGTTATGCAATGGATTTAGGCAAAAA TTCATTCTTAGTGTCAGCCACACAATTTTTTTTAATGCAGTATATTCACCTGTAAATAGTT TGTGTAAAATTTGACAAAAAAAGTATATTTACTATACTGTAAATATATGTGATGATATATT GTATTATTTTGCTTTTTTGTAAAGCAGTTAGTTGCTGCACATGGATAACAACAAAAATTT GATTATTCTCGTGTTAGTATTGTTAACTTCTTTTTGCGACTGCGTTACATCATTTAAAGAA AATGCTGTGTATTGTAAACTTAAATTGTATATGATAACTTACTGTCCTTTCCATCCGGGC CTAAACTTTGGCAGTTCCTTTGTCTACAACCTTGTTAATACTGTAAACAGTTGTACGCCA GCAGGAAAAATACTGCCCAACAGACAAAATCGATCATTGTAGGGGAAAATCATAGAAAT CCATTTCAGATCTTTATTGTTCCTCACCCCATTTTCCTCCTTGTGTATGTACTTCCCCCA CCCCCCTTTTTTTAAGTAAAATGTAAATTCAATCTGCTCTAAGAAAAAAAAAAAAAAAAAA AA. (SEQ ID NO:268; NM_000267.3), or a nucleic acid sequence that hybridizes to a nucleic acid sequence consisting of SEQ ID NO:268 under stringent hybridization conditions

In some embodiments, early growth response 1 (EGR1, TIS8, AT225, KROX-24, NGFI-A, ZNF225) comprises the amino acid sequence: MAAAKAEMQLMSPLQISDPFGSFPHSPTMDNYPKLEEMMLLSNGAPQFLGAAGAPEGSG SNSSSSSSGGGGGGGGGSNSSSSSSTFNPQADTGEQPYEHLTAESFPDISLNNEKVLVET SYPSQTTRLPPITYTGRFSLEPAPNSGNTLWPEPLFSLVSGLVSMTNPPASSSSAPSPAAS SASASQSPPLSCAVPSNDSSPIYSAAPTFPTPNTDIFPEPQSQAFPGSAGTALQYPPPAYPA AKGGFQVPMIPDYLFPQQQGDLGLGTPDQKPFQGLESRTQQPSLTPLSTIKAFATQSGSQ DLKALNTSYQSQLIKPSRMRKYPNRPSKTPPHERPYACPVESCDRRFSRSDELTRHIRIHT GQKPFQCRICMRNFSRSDHLTTHIRTHTGEKPFACDICGRKFARSDERKRHTKIHLRQKDK KADKSVVASSATSSLSSYPSPVATSYPSPVTTSYPSPATTSYPSPVPTSFSSPGSSTYPSPV HSGFPSPSVATTYSSVPPAFPAQVSSFPSSAVTNSFSASTGLSDMTATFSPRTIEIC. (SEQ ID NO:269; NP_001955.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:269)

In some embodiments, the nucleic acid sequence encoding Fos comprises the nucleic acid sequence: GAGAGATCCCAGCGCGCAGAACTTGGGGAGCCGCCGCCGCCATCCGCCGCCGCAGC CAGCTTCCGCCGCCGCAGGACCGGCCCCTGCCCCAGCCTCCGCAGCCGCGGCGCGT CCACGCCCGCCCGCGCCCAGGGCGAGTCGGGGTCGCCGCCTGCACGCTTCTCAGTG TTCCCCGCGCCCCGCATGTAACCCGGCCAGGCCCCCGCAACTGTGTCCCCTGCAGCT CCAGCCCCGGGCTGCACCCCCCCGCCCCGACACCAGCTCTCCAGCCTGCTCGTCCAG GATGGCCGCGGCCAAGGCCGAGATGCAGCTGATGTCCCCGCTGCAGATCTCTGACCC GTTCGGATCCTTTCCTCACTCGCCCACCATGGACAACTACCCTAAGCTGGAGGAGATG ATGCTGCTGAGCAACGGGGCTCCCCAGTTCCTCGGCGCCGCCGGGGCCCCAGAGGG CAGCGGCAGCAACAGCAGCAGCAGCAGCAGCGGGGGCGGTGGAGGCGGCGGGGGC GGCAGCAACAGCAGCAGCAGCAGCAGCACCTTCAACCCTCAGGCGGACACGGGCGA GCAGCCCTACGAGCACCTGACCGCAGAGTCTTTTCCTGACATCTCTCTGAACAACGAG AAGGTGCTGGTGGAGACCAGTTACCCCAGCCAAACCACTCGACTGCCCCCCATCACCT ATACTGGCCGCTTTTCCCTGGAGCCTGCACCCAACAGTGGCAACACCTTGTGGCCCGA GCCCCTCTTCAGCTTGGTCAGTGGCCTAGTGAGCATGACCAACCCACCGGCCTCCTCG TCCTCAGCACCATCTCCAGCGGCCTCCTCCGCCTCCGCCTCCCAGAGCCCACCCCTG AGCTGCGCAGTGCCATCCAACGACAGCAGTCCCATTTACTCAGCGGCACCCACCTTCC CCACGCCGAACACTGACATTTTCCCTGAGCCACAAAGCCAGGCCTTCCCGGGCTCGG CAGGGACAGCGCTCCAGTACCCGCCTCCTGCCTACCCTGCCGCCAAGGGTGGCTTCC AGGTTCCCATGATCCCCGACTACCTGTTTCCACAGCAGCAGGGGGATCTGGGCCTGG GCACCCCAGACCAGAAGCCCTTCCAGGGCCTGGAGAGCCGCACCCAGCAGCCTTCGC TAACCCCTCTGTCTACTATTAAGGCCTTTGCCACTCAGTCGGGCTCCCAGGACCTGAA GGCCCTCAATACCAGCTACCAGTCCCAGCTCATCAAACCCAGCCGCATGCGCAAGTAC CCCAACCGGCCCAGCAAGACGCCCCCCCACGAACGCCCTTACGCTTGCCCAGTGGAG TCCTGTGATCGCCGCTTCTCCCGCTCCGACGAGCTCACCCGCCACATCCGCATCCACA CAGGCCAGAAGCCCTTCCAGTGCCGCATCTGCATGCGCAACTTCAGCCGCAGCGACC ACCTCACCACCCACATCCGCACCCACACAGGCGAAAAGCCCTTCGCCTGCGACATCTG TGGAAGAAAGTTTGCCAGGAGCGATGAACGCAAGAGGCATACCAAGATCCACTTGCGG CAGAAGGACAAGAAAGCAGACAAAAGTGTTGTGGCCTCTTCGGCCACCTCCTCTCTCT CTTCCTACCCGTCCCCGGTTGCTACCTCTTACCCGTCCCCGGTTACTACCTCTTATCCA TCCCCGGCCACCACCTCATACCCATCCCCTGTGCCCACCTCCTTCTCCTCTCCCGGCT CCTCGACCTACCCATCCCCTGTGCACAGTGGCTTCCCCTCCCCGTCGGTGGCCACCAC GTACTCCTCTGTTCCCCCTGCTTTCCCGGCCCAGGTCAGCAGCTTCCCTTCCTCAGCT GTCACCAACTCCTTCAGCGCCTCCACAGGGCTTTCGGACATGACAGCAACCTTTTCTC CCAGGACAATTGAAATTTGCTAAAGGGAAAGGGGAAAGAAAGGGAAAAGGGAGAAAAA GAAACACAAGAGACTTAAAGGACAGGAGGAGGAGATGGCCATAGGAGAGGAGGGTTC CTCTTAGGTCAGATGGAGGTTCTCAGAGCCAAGTCCTCCCTCTCTACTGGAGTGGAAG GTCTATTGGCCAACAATCCTTTCTGCCCACTTCCCCTTCCCCAATTACTATTCCCTTTGA CTTCAGCTGCCTGAAACAGCCATGTCCAAGTTCTTCACCTCTATCCAAAGAACTTGATTT GCATGGATTTTGGATAAATCATTTCAGTATCATCTCCATCATATGCCTGACCCCTTGCTC CCTTCAATGCTAGAAAATCGAGTTGGCAAAATGGGGTTTGGGCCCCTCAGAGCCCTGC CCTGCACCCTTGTACAGTGTCTGTGCCATGGATTTCGTTTTTCTTGGGGTACTCTTGAT GTGAAGATAATTTGCATATTCTATTGTATTATTTGGAGTTAGGTCCTCACTTGGGGGAAA AAAAAAAAAGAAAAGCCAAGCAAACCAATGGTGATCCTCTATTTTGTGATGATGCTGTG ACAATAAGTTTGAACCTTTTTTTTTGAAACAGCAGTCCCAGTATTCTCAGAGCATGTGTC AGAGTGTTGTTCCGTTAACCTTTTTGTAAATACTGCTTGACCGTACTCTCACATGTGGCA AAATATGGTTTGGTTTTTCTTTTTTTTTTTTTTTGAAAGTGTTTTTTCTTCGTCCTTTTGGT TTAAAAAGTTTCACGTCTTGGTGCCTTTTGTGTGATGCGCCTTGCTGATGGCTTGACAT GTGCAATTGTGAGGGACATGCTCACCTCTAGCCTTAAGGGGGGCAGGGAGTGATGATT TGGGGGAGGCTTTGGGAGCAAAATAAGGAAGAGGGCTGAGCTGAGCTTCGGTTCTCC AGAATGTAAGAAAACAAAATCTAAAACAAAATCTGAACTCTCAAAAGTCTATTTTTTTAAC TGAAAATGTAAATTTATAAATATATTCAGGAGTTGGAATGTTGTAGTTACCTACTGAGTA GGCGGCGATTTTTGTATGTTATGAACATGCAGTTCATTATTTTGTGGTTCTATTTTACTTT GTACTTGTGTTTGCTTAAACAAAGTGACTGTTTGGCTTATAAACACATTGAATGCGCTTT ATTGCCCATGGGATATGTGGTGTATATCCTTCCAAAAAATTAAAACGAAAATAAAGTA. (SEQ ID NO:270; NM_001964.3), or a nucleic acid sequence that hybridizes to a nucleic acid sequence consisting of SEQ ID NO:270 under stringent hybridization conditions

In some embodiments, early growth response 2 (EGR2, CHN1, AT591, CMT1 D, CMT4E, KROX20) comprises the amino acid sequence: MMTAKAVDKIPVTLSGFVHQLSDNIYPVEDLAATSVTIFPNAELGGPFDQMNGVAGDGMINI DMTGEKRSLDLPYPSSFAPVSAPRNQTFTYMGKFSIDPQYPGASCYPEGIINIVSAGILQGV TSPASTTASSSVTSASPNPLATGPLGVCTMSQTQPDLDHLYSPPPPPPPYSGCAGDLYQD PSAFLSAATTSTSSSLAYPPPPSYPSPKPATDPGLFPMIPDYPGFFPSQCQRDLHGTAGPD RKPFPCPLDTLRVPPPLTPLSTIRNFTLGGPSAGVTGPGASGGSEGPRLPGSSSAAAAAAA AAAYNPHHLPLRPILRPRKYPNRPSKTPVHERPYPCPAEGCDRRFSRSDELTRHIRIHTGH KPFQCRICMRNFSRSDHLTTHIRTHTGEKPFACDYCGRKFARSDERKRHTKIHLRQKERKS SAPSASVPAPSTASCSGGVQPGGTLCSSNSSSLGGGPLAPCSSRTRTP. (SEQ ID NO:271; NP_000390.2), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:271)

In some embodiments, the nucleic acid sequence encoding EGR2 comprises the nucleic acid sequence: AACTGAGCGAGGAGCAATTGATTAATAGCTCGGCGAGGGGACTCACTGACTGTTATAA TAACACTACACCAGCAACTCCTGGCTTCCCAGCAGCCGGAACACAGACAGGAGAGAGT CAGTGGCAAATAGACATTTTTCTTATTTCTTAAAAAACAGCAACTTGTTTGCTACTTTTAT TTCTGTTGATTTTTTTTTCTTGGTGTGTGTGGTGGTTGTTTTTAAGTGTGGAGGGCAAAA GGAGATACCATCCCAGGCTCAGTCCAACCCCTCTCCAAAACGGCTTTTCTGACACTCC AGGTAGCGAGGGAGTTGGGTCTCCAGGTTGTGCGAGGAGCAAATGATGACCGCCAAG GCCGTAGACAAAATCCCAGTAACTCTCAGTGGTTTTGTGCACCAGCTGTCTGACAACAT CTACCCGGTGGAGGACCTCGCCGCCACGTCGGTGACCATCTTTCCCAATGCCGAACT GGGAGGCCCCTTTGACCAGATGAACGGAGTGGCCGGAGATGGCATGATCAACATTGA CATGACTGGAGAGAAGAGGTCGTTGGATCTCCCATATCCCAGCAGCTTTGCTCCCGTC TCTGCACCTAGAAACCAGACCTTCACTTACATGGGCAAGTTCTCCATTGACCCTCAGTA CCCTGGTGCCAGCTGCTACCCAGAAGGCATAATCAATATTGTGAGTGCAGGCATCTTG CAAGGGGTCACTTCCCCAGCTTCAACCACAGCCTCATCCAGCGTCACCTCTGCCTCCC CCAACCCACTGGCCACAGGACCCCTGGGTGTGTGCACCATGTCCCAGACCCAGCCTG ACCTGGACCACCTGTACTCTCCGCCACCGCCTCCTCCTCCTTATTCTGGCTGTGCAGG AGACCTCTACCAGGACCCTTCTGCGTTCCTGTCAGCAGCCACCACCTCCACCTCTTCC TCTCTGGCCTACCCACCACCTCCTTCCTATCCATCCCCCAAGCCAGCCACGGACCCAG GTCTCTTCCCAATGATCCCAGACTATCCTGGATTCTTTCCATCTCAGTGCCAGAGAGAC CTACATGGTACAGCTGGCCCAGACCGTAAGCCCTTTCCCTGCCCACTGGACACCCTGC GGGTGCCCCCTCCACTCACTCCACTCTCTACAATCCGTAACTTTACCCTGGGGGGCCC CAGTGCTGGGGTGACCGGACCAGGGGCCAGTGGAGGCAGCGAGGGACCCCGGCTGC CTGGTAGCAGCTCAGCAGCAGCAGCAGCCGCCGCCGCCGCCGCCTATAACCCACACC ACCTGCCACTGCGGCCCATTCTGAGGCCTCGCAAGTACCCCAACAGACCCAGCAAGA CGCCGGTGCACGAGAGGCCCTACCCGTGCCCAGCAGAAGGCTGCGACCGGCGGTTC TCCCGCTCTGACGAGCTGACACGGCACATCCGAATCCACACTGGGCATAAGCCCTTCC AGTGTCGGATCTGCATGCGCAACTTCAGCCGCAGTGACCACCTCACCACCCATATCCG CACCCACACCGGTGAGAAGCCCTTCGCCTGTGACTACTGTGGCCGAAAGTTTGCCCG GAGTGATGAGAGGAAGCGCCACACCAAGATCCACCTGAGACAGAAAGAGCGGAAAAG CAGTGCCCCCTCTGCATCGGTGCCAGCCCCCTCTACAGCCTCCTGCTCTGGGGGCGT GCAGCCTGGGGGTACCCTGTGCAGCAGTAACAGCAGCAGTCTTGGCGGAGGGCCGCT CGCCCCTTGCTCCTCTCGGACCCGGACACCTTGAGATGAGACTCAGGCTGATACACCA GCTCCCAAAGGTCCCGGAGGCCCTTTGTCCACTGGAGCTGCACAACAAACACTACCAC CCTTTCCTGTCCCTCTCTCCCTTTGTTGGGCAAAGGGCTTTGGTGGAGCTAGCACTGC CCCCTTTCCACCTAGAAGCAGGTTCTTCCTAAAACTTAGCCCATTCTAGTCTCTCTTAG GTGAGTTGACTATCAACCCAAGGCAAAGGGGAGGCTCAGAAGGAGGTGGTGTGGGGA CCCCTGGCCAAGAGGGCTGAGGTCTGACCCTGCTTTAAAGGGTTGTTTGACTAGGTTT TGCTACCCCACTTCCCCTTATTTTGACCCATCACAGGTTTTTGACCCTGGATGTCAGAG TTGATCTAAGACGTTTTCTACAATAGGTTGGGAGATGCTGATCCCTTCAAGTGGGGACA GCAAAAAGACAAGCAAAACTGATGTGCACTTTATGGCTTGGGACTGATTTGGGGGACA TTGTACAGTGAGTGAAGTATAGCCTTTATGCCACACTCTGTGGCCCTAAAATGGTGAAT CAGAGCATATCTAGTTGTCTCAACCCTTGAAGCAATATGTATTATAAACTCAGAGAACAG AAGTGCAATGTGATGGGAGGAACATAGCAATATCTGCTCCTTTTCGAGTTGTTTGAGAA ATGTAGGCTATTTTTTCAGTGTATATCCACTCAGATTTTGTGTATTTTTGATGTACACTGT TCTCTAAATTCTGAATCTTTGGGAAAAAATGTAAAGCATTTATGATCTCAGAGGTTAACT TATTTAAGGGGGATGTACATATATTCTCTGAAACTAGGATGCATGCAATTGTGTTGGAA GTGTCCTTGGTGCCTTGTGTGATGTAGACAATGTTACAAGGTCTGCATGTAAATGGGTT GCCTTATTATGGAGAAAAAAAATCACTCCCTGAGTTTAGTATGGCTGTATATTTCTGCCT ATTAATATTTGGAATTTTTTTTAGAAAGTATATTTTTGTATGCTTTGTTTTGTGACTTAAAA GTGTTACCTTTGTAGTCAAATTTCAGATAAGAATGTACATAATGTTACCGGAGCTGATTT GTTTGGTCATTAGCTCTTAATAGTTGTGAAAAAATAAATCTATTCTAACGCAAAACCACT AACTGAAGTTCAGATAATGGATGGTTTGTGACTATAGTGTAAATAAATACTTTTCAACAA TA. (SEQ ID NO:272; NM_000399.5), or a nucleic acid sequence that hybridizes to a nucleic acid sequence consisting of SEQ ID NO:272 under stringent hybridization conditions

In some embodiments, Sp3 transcription factor (SP3, SPR2) comprises the amino acid sequence: MGPPSPGDDEEEAAAAAGAPAAAGATGDLASAQLGGAPNRWEVLSATPTTIKDEAGNLVQ IPSAATSSGQYVLPLQNLQNQQIFSVAPGSDSSNGTVSSVQYQVIPQIQSADGQQVQIGFT GSSDNGGINQESSQIQIIPGSNQTLLASGTPSANIQNLIPQTGQVQVQGVAIGGSSFPGQTQ VVANVPLGLPGNITFVPINSVDLDSLGLSGSSQTMTAGINADGHLINTGQAMDSSDNSERT GERVSPDINETNTDTDLFVPTSSSSQLPVTIDSTGILQQNTNSLTTSSGQVHSSDLQGNYIQ SPVSEETQAQNIQVSTAQPVVQHLQLQESQQPTSQAQIVQGITPQTIHGVQASGQNISQQA LQNLQLQLNPGTFLIQAQTVTPSGQVTWQTFQVQGVQNLQNLQIQNTAAQQITLTPVQTLT LGQVAAGGAFTSTPVSLSTGQLPNLQTVTVNSIDSAGIQLHPGENADSPADIRIKEEEPDPE EWQLSGDSTLNTNDLTHLRVQVVDEEGDQQHQEGKRLRRVACTCPNCKEGGGRGTNLG KKKQHICHIPGCGKVYGKTSHLRAHLRWHSGERPFVCNWMYCGKRFTRSDELQRHRRTH TGEKKFVCPECSKRFMRSDHLAKHIKTHQNKKGIHSSSTVLASVEAARDDTLITAGGTTLILA NIQQGSVSGIGTVNTSATSNQDILTNTEIPLQLVTVSGNETME. (SEQ ID NO:273; NP_001017371.3), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:273)

In some embodiments, the nucleic acid sequence encoding SP3 comprises the nucleic acid sequence: ACTCAGCCGTCACCGCTCGCTCTGCTGGCCGCTACCTGCAGCAAGATAGGGCCGCCA TCGCCGGGCGACGACGAGGAGGAGGCGGCCGCCGCAGCCGGGGCCCCCGCCGCCG CCGGAGCGACAGGTGATTTGGCTTCTGCACAGTTAGGAGGAGCACCAAACCGATGGG AGGTTTTGTCAGCCACACCTACAACTATAAAAGATGAAGCTGGTAATCTAGTCCAGATT CCAAGTGCTGCTACTTCAAGTGGGCAGTATGTTCTTCCCCTTCAGAATTTGCAGAATCA ACAAATATTTTCCGTTGCACCAGGATCAGATTCATCAAATGGTACAGTGTCCAGTGTTC AATTCAAGTGATACCACAGATCCAGTCAGCAGATGGTCAGCAGGTTCAAATTGGTTTCA CAGGCTCTTCAGATAATGGGGGTATAAATCAAGAAAGCAGTCAAATTCAGATCATTCCT GGCTCTAATCAAACCTTACTTGCCTCTGGAACACCTTCTGCTAACATCCAGAATCTCATA CCACAGACTGGTCAAGTCCAGGTTCAGGGAGTTGCAATTGGTGGTTCATCTTTTCCTG GTCAAACCCAAGTAGTTGCTAATGTGCCTCTTGGTCTGCCAGGAAATATTACGTTTGTA CCAATCAATAGTGTCGATCTAGATTCTTTGGGACTCTCGGGCAGTTCTCAGACAATGAC TGCAGGCATTAATGCCGACGGACATTTGATAAACACAGGACAAGCTATGGATAGTTCAG ACAATTCAGAAAGGACTGGTGAGCGGGTTTCTCCTGATATTAATGAAACTAATACTGAT ACAGATTTATTTGTGCCAACATCCTCTTCATCACAGTTGCCTGTTACGATAGATAGTACA GGTATATTACAACAAAACACAAATAGCTTGACTACATCTAGTGGGCAGGTTCATTCTTCA GATCTTCAGGGAAATTATATCCAGTCGCCTGTTTCTGAAGAGACACAGGCACAGAATAT TCAGGTTTCTACAGCACAGCCTGTTGTACAGCATCTACAACTTCAAGAGTCTCAGCAGC CAACCAGTCAAGCCCAAATTGTGCAAGGTATTACACCACAGACAATCCATGGTGTGCAA GCCAGTGGTCAAAATATATCACAACAGGCTTTGCAAAATCTTCAGTTGCAGCTGAATCC TGGAACCTTTTTAATTCAGGCACAGACAGTGACCCCTTCTGGACAGGTAACTTGGCAAA CGTTTCAAGTACAAGGGGTCCAGAACTTGCAGAATTTGCAAATACAGAATACTGCTGCC CAACAAATAACTTTGACGCCTGTTCAAACCCTCACACTTGGTCAAGTTGCGGCAGGTGG AGCCTTCACTTCAACTCCAGTTAGTCTAAGCACTGGTCAGTTGCCAAATCTACAAACAG TTACAGTGAACTCTATAGATTCTGCTGGTATACAGCTACATCCAGGAGAGAATGCTGAC AGTCCTGCAGATATTAGGATCAAGGAAGAAGAACCTGATCCTGAAGAGTGGCAGCTCA GTGGTGATTCTACCTTGAATACCAATGACCTAACACACTTAAGAGTACAGGTGGTAGAT GAAGAAGGGGACCAACAACATCAAGAAGGAAAAAGACTTCGGAGGGTAGCTTGCACCT GTCCCAACTGTAAAGAAGGTGGTGGAAGAGGTACCAATCTTGGGAAAAAGAAGCAACA CATTTGTCATATACCAGGATGTGGTAAAGTCTATGGGAAGACCTCACATCTGAGAGCTC ATCTGCGTTGGCATTCTGGAGAACGCCCTTTTGTTTGTAACTGGATGTACTGTGGTAAA AGATTTACTCGAAGTGATGAATTACAGAGGCACAGAAGAACACATACAGGTGAGAAGAA ATTTGTTTGTCCAGAATGTTCAAAACGCTTTATGAGAAGTGACCACCTTGCCAAACATAT TAAAACACACCAGAATAAAAAAGGTATTCACTCTAGCAGTACAGTGCTGGCATCTGTGG AAGCTGCGCGAGATGATACTTTGATTACTGCAGGAGGAACAACGCTTATCCTTGCAAAT ATTCAACAAGGTTCTGTTTCAGGGATAGGAACTGTTAATACTTCCGCCACCAGCAATCA AGATATCCTTACCAACACTGAAATACCTTTACAGCTTGTCACAGTTTCTGGAAATGAGAC AATGGAGTAAATATTACACAAATACTTATTCATTGTGGTTATTTTTATACAGTAGTGAGAA GAATATTGTTCCTAAGTTCTTAGATATCTTTTTATTGATGTGCAAAAATTTTTGGATTGAC AGTAACTTGGTTATACATGACACTGAAATGCCTTACTTTGTATGATATTCCATAGTATATT AAAAATGGTAAAATTGCATGGGTTTTGTAGGTACTTTTGGAATCTAGAAGAAATGAAATT TTACCAAGTTATATAAAGAGAAAATTGAATTTAACAATGCGAATGGTAGTCTAACCAAAT GCATCAATCCTGTGTGGTTTAGTGTAAAAATGAGAACATGTTGGTATTTATCTATTGTAA GATAAAAAAGCTGGTGGGTGAAAGAAATCATGTTATGATAAAAAATTTTGTAATTTTCTT GATGACTGGAATTTTTATTATGCATAACTGACAAATCAAGTTTCCAAGCAAATGTTACAT AGTGTAGGCTTTACTTAGCTTATCAATTTGTCATTTTGAAGCTAATTATTTTAATTAGGTT AACTATGTACAATATTTTAAGCATTACTCTTGTAAGATTTTGAAAACTACATTTTAACATG GAACTCTAGGGATAGTCACCTTTTAAATCCTGTTGAAAAGCCATGTTTAAGATTTAATTT GCCAAAATAATGTCTTGTTAATATTCTTTCAATAACGAAGTTGGGCAATATAACCAATGT TTAAAAAAGTTTAAAATGTATAAGTTGAGGCATTTGGGTGGTAAGAGAATGTTATAGTGA ATTATCCCTTTTCTTGACTATTGGAGGACCAAAAAAATAAGGTGTATTGCGTCTTAGCAG TGATTTTTATCCAATCTTGTTTCCAAAAACCATGTCTCCCAGGGCCTTAAAAGCCATCAT GTAAATTACCAGTAAAGTGTAACATATGCAAACATAACAAAATCACTTCCATAGTGACGA TACTCCAACCATATGGATATTAGTCATAGAAGAACTAGAGGTTTTATGATATTTTTTTAAG TCTTTTTTTTTTTTGTCTAGGTAGTCAGTCTGCACTTAAATATCAATCATTTTCCTTTTTTG CTTCTTCCCTTAAAATTTATATGTATCCAGTACATTTAATTGAGAAGCGTATGTTTTTTAT TATGCTGTATTTTCTTTTTATTTTTTAATTATTGTTTATATTTTCAATTCAAAAATGTACAAA ATAAAGTTACATTGCTGGTCTTGTAAGAGCTATACAGTTTTCCTAAATGTATACCTGTAA CTGCAGCAGTTCACCTATTTCAAAAATTTGGAATTCTGTTCATTTGTTATTCTTAAGACCA CCTCAAATTTAAAGGCTACCTTATTGTACGTTTAAAGTGTATTATAACAGTGTGGTAGTT AATAAAACACTATTTTTTTTTCTTTTGAGTTTGTTGTATTCCTATGCATAAAAAATATTGCA GTGGTATGGGGTAAGAATTGGTGGTTTATTTTTCTTCAACTTGGCTTTTTATTTTTAGATT CTTGATTTTAGACACTGAATTGTAAACAGGCATTTATTTGAAGAAGAGATATATAGAGAC ACTGGTCATTTACTAATTTTTTACCTAGAGTAAATAAGAATGAGCTTATTAAATAAAATTT TTGAAAAAAAGTCTTAGCCCTTAGCCCACATTGATTCATATCAGTTTTATCAGTACCATT TTGCAATTTTTTTGTTTTCCGTTTTAAAGCAATGCAGAATATTTTGATTTATCGAAAACCT GAATTTACATTAAGACTCCTGAAAATGATAAGACAAGCGTTGGTAACCATGGCAGGAGT TACTTGAAAAAGTTGCCTTTGAATTTGCATGTGTTTCATCATTATAAAGGCAGAGTAGGA GGAAAGAGTATTAATGTGATTGTGTATTGTAGATGTTTTAAAGTAAAAATCAAGTTTCTTA ACACATGTATACAGTGGGGGTAAAGATGTCCATTTTCTGTTTTCCAGGCCCAGTCTGAC TCTGTCTGTAATACCTGATTGCATTGGAGAACTCTAGACACGCATAAACATGGACAGTT TTTCTAAATGTGAGACTTAAGCCTGTGATGTAAAATAGGAAGTTCTACTTGGAATAATAT AAAGGAACCACTAGAATTTACAATTATTTTGAAGTTACAGGGATTAGATTTTGAATCTTA AAATCCTTTAGGTAATTTTTAGAATTTTTAAATTAAGATTAATGTGAAGAGAATTAAGTGA GCAGCAGGTGGTTCACTTGAGCAAACTGCCCATTAAGTCAGATTAAACCATTTGAATGA TAGTAGAGATGTTTTAAGTAATACTGGATTTTTACAGTAAGTTTATGGTGTACTTTGAATC GATAGGTGTCCATGCATATTTTATGAATTCGTGGAGAATGAATGCAATGAAGAAAGTAA GTAGTCTTGAAAATTATTTAAATAAAGTTGTAGATTTTTTTAGTGCCCCCTAGGAATATAT TAGTGAACTTTGGAACTTTTACCAAAGTTATGTAACCTCAGTGTAGATAATTTTAAATATT TCTATTTTTATATTTTAAAATGTTGAATATACTCTGGAAACAACATTTGAAGATTTGCTCT GATGTCAACTTTTTCTGGTTATAAAACCTATTAGTATTGTGTATAATTCTCAGGGTAGGT ATACTCTAATAGGTGTTTTGTCAATTGCTTTATTTTTGTAAAGGCTAGAGTTAGTGCATAT TGAATATATTTATGTACAAATAATTCCTGTTGTAACATTTAGTGGACGCGATTATCTGTAT ACCTCAAATTTTAATTTAAGAAAGTATCACTTAAAGAGCATCTCATTTTCTATAGATTGAG GCTTAATTACTGAAAAGTGACTCAACCAAAAAGCACATAACCTTTTAAAGGAGCTACAC CTACCGCAGAAAGTCAGATGCCCTGTAAATAACTTTGGTCTTTCAAAATAGTGGCAATG CTTAAGATACTTAAAAATACACATACATATAAGCTGAAAGCATGTCAAGCCTATTTCATA GAAAATAGTTCTTAAACAGTATTGTTCATTAGAAATTGCTGGGGAGCATTTTAGAGATTC CATAGGCCTGGTAGCCACCTCAGCTTACTAAATCAACATCTCTGGAGATGGAGTGTATA TGTGTTTTGAAACAGCTGCTCAAGTGGTTCTGTTAAACACTCCTGCGTTACAATCACTTA AAATAGAGCAAGCATCCCCTTAGGCTCTTAATTGTAGTTTAAATTCCAGTACTGCCTACT CAGACCCAAAAGTTTTGTTTTATGAAAAATTTGTATTGTGTTCAATATTGTTTGAAATTTG GGGTTGTTGCATAAATGATTATGGAATAACATTTGGTTTTAAAATAATATAAACTGACAT GTTATGCTACCTGTTACACAATTTGGTTTTCAGTTTTAATTATATGAAGCTGGTAACAATC GTTTTTGTTGTAAAAGAATTATTTTTCACTAAACAGTATGTTTAAAACTGACTGCCATGAA TGAGTACTAAGTCTTTTGTTGTCTGACAATAAGCACAAAACAAATTAATTGATATCTTTG GTACAAATTTGATATTTTTGTGAAATCACCTCATAATTTTATTTAGTGTTGAAGAAACAAC ATTGGCCCCTTGCCTTGTTCAGACAGATTTGCGTATAATGTAAATATATATATTAGTGGC AGAAACAAAGTACTGTAGAAACCTAGAAGAGGGAGAATTCTGCATGAAGTCTGGGAAA CTCAGCAGAAATGGCGTTTGCAAAGGTACAGTGTGGTATGTTGGGTGATGGGGGACTT TGTGAGGGACAGAGTGGAAAAATTTTTAAGAGGGGCTGCCAAATTGCAAAAGAGAAGA ATTTTTTTTGTCAGTGATTCCTAGTCTTTTTGGAATCCTACACCTTCTCCCCAGAAAAATT CATTCTAGTCCAGGGTTTATTGTTTGTTTTTTCCCTATATTTGACAGGGTCTGTCTCTGT CACACAGTGCAGTGGTGCAATCATGGCTTACTGCAGACTCCACTTACTGGTCTCAAGC AATCCTCCCCACTTCGGCCTCTTAAGTAGCTTGGACCACAGGTGCACACCACCATGCT CAGTTAATTTTAATTTGTTTTGTAGAGATGGGGTCTCACTATATTGCCTAGGCTGGTCTT GAATTCCTGGGCTCAAGTGATCCTTCCACCTCTGCCTCCCAAAGTGTTGGGATTATAGG CGTGAGCCACCACGCTTGGCCAAATCTGGAGTCTAAAGGGTATACAAGGTTTTTTTTTT TCCCATGTTGGCTTTTATTTGATGTTATAAAGTCTTCATGATATGAATATATTTTTTAAAA AGGTGTCATGTATATTGTCATGTAACATGATTCATGCCACTTCATTGTATTAAATACGTTT AAGATACAGTTTTTGATGTTAAGAATGATATCCTGAGAAGGCCTATCTCCTTCAGTCCTT TTCCCCATTTTTCACCTTTTAACTCTTGCTTCTGAATCTATGCTAAGCTACTTACTAGATG TGTGATATTGGTAGTTTTCTTACCTTAAAACCTCATTTTCATCATCCATGAAGAGGCTAA CAATAGTACTCCCTGTTGATAGGATTGTTGTATTAAAATATAAGCATTCAGCATAGTGCC TGACTTAACGGTAGAGATTCAAATGGTAACCTCCTTCCAATGCCTTCCTCCCCCCTTAA CTCTGGAGATTCCATTTTTCTGTAGTGAAGTGTTTTAGAATTTTCATTGTTTGACTATGGT TCATACATTGATTTTTCCAAAATGAGAAGAAGTCTTATTTCTAATGCAAAAATGTGAAAA GAACAGTTGGAACCTAAAGTGATAGTGGATGAAGTGTCTGAAGTGACTGCCCTATCAG AAAAGGATGGAATAAGAAGAAAAGACTGAAGCACCTAAATGTGTATTTTCTTGAGGAAA TTACACCCCTGTGTAAGAGTCTATCCTATTTGAACATTTCTAAAAACCAGCCGAAGAATC TTCAGGTTCATTGCGACTGAAAGATAAAGTCTAGCACTGAAGTGGTTTTTAAGATTAGG AAAGGCCATCAGAGAAATGCAGTTATTTCTCCCCTCCATCCTTCCCCCACAAAAAAAAG TCTAAGCCTCCGATTAATCGACAACAACGAAATACAGGAAGTATTGGCTTAGTGACCTT TTAGGATATCTGTCCGACTTACATCCCTTCTCTGAGCATTACTTTCTGCCACCTTCCGTG AGGAGCTGTTCAGAAATCAGACAAGGAGTGGACATCTGGTCAAAGTTGAGCCAATTAG ATGATTTCTCCTGAGAATTTGGAATTGGAAAACTGATGTCTTGGGAGGTCACATCTGGA ACATACATGGGAGGCAGAGAAAGCAAAGATGACATTCAACCCCAGAAGAAAAAGAAAA TGGCTTTCTTGATTCCTGATAACTTGGCAGTGTTTGTTCCAGTGTCTTGCATTTCACTAT CTGCATTTCATCCTCCTGGGGTTCCACAAGATAGCCTTAAAACCTTACAGAGAGACTCC CCAACTTAGTTTTTTCTTAAGTCAGAAGGCTGTTACTTGCTACCAAATAATGCCAACTAA GACAACTGAGGTACAGTATATTCTCTCGTAAAACACCATAGAGTTGATGACTTGATCCT ATAGAAAAGGGTCCCCAACCCCTGGGCCACAGACCGGTACTGGTCCCTGGTCTGTGA GGAACCACCTGGGCCACAGACTGGTCCTGGTCCCTGGTCTGTGAGGAACCAGGCCAT ACAGCAGGAGGTAAGCGAGCATTCCTGCCTGAGCTCTGCCTCCTGTCAGATGAGCCAC AACATTAGATTCTCATAGGAGTGCGAACCCCGTTGTGAACCCCGCATGCGAGGGATCT AGGTAGTGCCCTCCTTAAGAGAATCTAATGTCTGATGATCTGAGGTAGAGCAGTTTCAT CCTACAACCATCCCCACCCCCCGACCCCCATCCATGAAAAAATTGTCTTCCTCAAAACT AGTCCCTGGTGCCAAAAAGTTTGGGGACCGCCGTCCTAGGAAATACCAACAAAAAGTC ACGTTTATTGCCTGCCAAGTGAGTCAGGTTTAGACAAGGCAGAACTTAGTATATAGCTT GGATGAATCAGAAAGGGTGGGCCTTGAAAGACTGGTAGAATGTGTCCAAGAAAACTGA TTAAAAGCCTTAGAAGTCCCTCTACAGTTTCAGGACGCCCTCTTAACAGATGGCTGTAT CTTTATCTGTGGTTGGTCTTGTGGAATACTGACTTTCTGCCAGCATTAGCCTGTGTTTCT ATGTCACACCAAGTCACGTATATTTTCATTGTGTGTTAGGACTCAGAATAGGTTTGGGT CTTTCTTTTAATTATGTATACCCAATTACTTAGTCCTCCTCACACCCTGATCCTTTAAAAA TTCTTAAGTTGTGTTAATTGCATTTTTCTCAATTCTACTCCTAGTAACTCTGTGTGTTTTT TTCCATCTTTATTCATTTAGTAAAGTTGAAACCTTTCATAGCATAATATAGTGATTAAGTG CATAGGTTTGGGACTTAGACACACATGGGCTTGCTTGTCTCCCAGTTCTGCCGCTTACC ACCTATGTGACCTTTCACTCTTTACCTTTCCACTCTTAGCCTGTTCTCTAACTATAAAATG AGAATGATGCTAGCGTCATCTTCATAGAGTGATTATGGAGATAAATAAGTTAATGCATGT AAACCACTTTCCATAATGCCTGGCACATATTATATTATGTATTAGTAAATGTAAGCACTG GAAGAATCAGTAGTCTTCCTACACTTGAAAATAGTTGTCTTCAACTATTGTTGTATTAAT GAGTTAATTCACCTTGTTAAGTCACCTTGATCCACCTAATTGTCAGCCAACATTTATTAA CGAAACTGCTAGTATTCGTATTGTGCCTTTTTTATTATGAAACATTTCAAGAATACAGAA AAATGCAGAAAACAATGTAACAAATACCTAAGTATCTATCACCCAACTTTTATAGTCTTAT TTTTACCTATATATTTTTTCTTTTAGAAAGGAATGATCACAGTTGTGCCTGAAATCTGTAT AGCCCTGTCTCATTTCATTTGTCTCCCTTTACCCACATAAGTAATTGCAGCTCTGAATTT GATGTTTATCAGAATGATACCATGAGTGGTTTTATATCCTTATTATATGTATGCATACCTA GGATTGTTTTTCTGATTTTGAAACTTCATATAAATGGTATCATTCTGTACAAAACCTTCTG GGACTTGTTTTACCACTCATGTTTTTGAGATGTATCCACACTACCATCTTTCTCTAAATC ATTCATTTAAATGCTGTAGTTCATTGAATAAGTTTATCACAATGTATCCATTTTCTTCTTG ATGAGCATCTCAGTTATTTCCAATTTTTGACTTATAAACAATGCTGCAATGAAATAATTGA CATATTTCCTTGTGTACATGTGAGCATTTCTTGAGATATGTATCTAGAACTTGAATCACT GGCCAACAGGATATTCTTAATCTTCAGTTTCACCAGATACTTCCAAATTGATCTCCATAA CATGCATACCAAATTATATTCCTACCAGCCATTTATAGGAGTTCACATTTTTTCCATACC CTCCCAATCCTGCCTTGGCAGATATGTTTTTTCCAATGAATAACAATGAAATTTTGTTTT GGGATTTGACAAAATGATTCTAGATTCATCTGGAAGAAAAGCAAGTATAAGTAAGAAATT TAAAAGGGACCTGAAAAACTAAGCAATGGATATATTTAAAAATTGGTACCAGTAGGGAT AACCAAATATTTGTTAACTGTAGCAGAAACAATGCATGTTTTTCTCACTGGTTCCACCTT CTATCTCTCAGTCAAGCCCTGCCCAGGTGTGGTAGTCCTCATAGTCTCTCACTGTAGG GGTCTTCTCACATAGTAGACCACTCTCTTGAGTTGAATATTGAAAAGAAGGCTTGGCCG GGGGCAGTGGCTCATGCCTGTGGTCCCAGCACTTTGGGAAGCTGAGTCGAGCGGATT GCTTGAGGTCAGCAGTTCGAGAGCAGCCTGACTAACATGGTGATGAAAAGTACAAAAT TAGCTGGGGGTGGTGGCGCATGCCTGCAATCCCAGCTACTCGGGAGGCTGAGGCACG AGAATCGCTTGAACGCAGGAGGCATAGGTTGCAGTGAGCCGAGATTGCACCACTGCAC TCCAGCCTGGGCGACAGAGCAAGATTTCGTCTCAAAAAAAAAAAAAAAAAAAAAAAAGG CTTAAGTCCAACTAACTGTTTCCCTGCTGTTCATTTTATCCAACGGCTGGGGATATAGG CTACTGCACATGATGGGTTGATGGATGTGAAATGATAACTCATGACTTTAATTCTAATTT TTCTATATTCTGGTGAGGTTGAGCATATTTTTACAAGTTCATAATCCATTTTCCTCTGATG GGAGTTGCCTGTTTGAATCCTTTGCCCACTTGCTTTTTGGGGTTATTTGCAATTTTTTGC ATTGATCTTCAAACATTCTTAATTAATCCCAGGTACATATTATTTTATATATAAGATATATT TTAGAGATATATAGGCTGATAGTATCTTTTCTCAGTCTAAGGCTTATCTTTTAATTTTGTT TATGGTGTTTTGTGTGTGTTGTTTTGCAGAAGTTTTATTGTATCTGAATTGTGGCTTTTGT GTCTTAAGAAATTCTTTACCTACTGTCATAAAAATTTTTGTCTAAAAATTTTATGATTTTGC CTTTCTATTTATCTTGTTAATTCACTTTGAAATTACTTTTGTTTATGGCATAAAGTCCATTA TTTTCCATATGGATAATCAGTTGTCTTAGCAGTTTATTGAATGGTCCAACGTTTCCCTGC TGATTATAATGCCCTCTCTGTTATTTATCCTAAATTCTTGACATCCGGTAAGGAAGTCTC CTCTGCCCACCCTTCTCTGTATTTCAAAATGGTTTTGACTGTTCTTGACCCCTTGCTCTT TCTGTGAACTTTAGGATTAAATGATTATGTAGAATTAAACAATCGGGTTAAGGTTTTTTAT TAGAATTACATTGAATCTATAGATTAATTTGGGGAAAGAATGTTATCTTTCTATCCATGAT AATTCTGTATCTCTCCATTAATTCAAGTCTTTAATGGTTTTTAAAAATAAATTATTGTCTCT TTGAAAA. (SEQ ID NO:274; NM_001017371.5), or a nucleic acid sequence that hybridizes to a nucleic acid sequence consisting of SEQ ID NO:274 under stringent hybridization conditions

In some embodiments, SIX homeobox 1 (SIX1, BOS3, TIP39, DFNA23)) comprises the amino acid sequence: MSMLPSFGFTQEQVACVCEVLQQGGNLERLGRFLWSLPACDHLHKNESVLKAKAVVAFH RGNFRELYKILESHQFSPHNHPKLQQLWLKAHYVEAEKLRGRPLGAVGKYRVRRKFPLPR TIWDGEETSYCFKEKSRGVLREWYAHNPYPSPREKRELAEATGLTTTQVSNWFKNRRQR DRAAEAKERENTENNNSSSNKQNQLSPLEGGKPLMSSSEEEFSPPQSPDQNSVLLLQGN MGHARSSNYSLPGLTASQPSHGLQTHQHQLQDSLLGPLTSSLVDLGS. (SEQ ID NO:275; NP_005973.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:275)

In some embodiments, the nucleic acid sequence encoding SIX1 comprises the nucleic acid sequence: AAGTTCCCCGGCAACTAGCAGCATCCACCGGGCGGGAGGTCGGAGGCAGCAAGGCCT TAAAGGCTACTGAGTGCGCCGGCCGTTCCGTGTCCAGAACCTCCCCTACTCCTCCGCC TTCTCTTCCTTGGCCGCCCACCGCCAAGTTCCGACTCCGGTTTTCGCCTTTGCAAAGC CTAAGGAGGAGGTTAGGAACAGCCGCGCCCCCCTCCCTGCGGCCGCCGCCCCCTGC CTCTCGGCTCTGCTCCCTGCCGCGTGCGCCTGGGCCGTGCGCCCCGGCAGGCCCCA GCCATGTCGATGCTGCCGTCGTTTGGCTTTACGCAGGAGCAAGTGGCGTGCGTGTGC GAGGTTCTGCAGCAAGGCGGAAACCTGGAGCGCCTGGGCAGGTTCCTGTGGTCACTG CCCGCCTGCGACCACCTGCACAAGAACGAGAGCGTACTCAAGGCCAAGGCGGTGGTC GCCTTCCACCGCGGCAACTTCCGTGAGCTCTACAAGATCCTGGAGAGCCACCAGTTCT CGCCTCACAACCACCCCAAACTGCAGCAACTGTGGCTGAAGGCGCATTACGTGGAGG CCGAGAAGCTGCGCGGCCGACCCCTGGGCGCCGTGGGCAAATATCGGGTGCGCCGA AAATTTCCACTGCCGCGCACCATCTGGGACGGCGAGGAGACCAGCTACTGCTTCAAGG AGAAGTCGAGGGGTGTCCTGCGGGAGTGGTACGCGCACAATCCCTACCCATCGCCGC GTGAGAAGCGGGAGCTGGCCGAGGCCACCGGCCTCACCACCACCCAGGTCAGCAACT GGTTTAAGAACCGGAGGCAAAGAGACCGGGCCGCGGAGGCCAAGGAAAGGGAGAAC ACCGAAAACAATAACTCCTCCTCCAACAAGCAGAACCAACTCTCTCCTCTGGAAGGGG GCAAGCCGCTCATGTCCAGCTCAGAAGAGGAATTCTCACCTCCCCAAAGTCCAGACCA GAACTCGGTCCTTCTGCTGCAGGGCAATATGGGCCACGCCAGGAGCTCAAACTATTCT CTCCCGGGCTTAACAGCCTCGCAGCCCAGTCACGGCCTGCAGACCCACCAGCATCAG CTCCAAGACTCTCTGCTCGGCCCCCTCACCTCCAGTCTGGTGGACTTGGGGTCCTAAG TGGGGAGGGACTGGGGCCTCGAAGGGATTCCTGGAGCAGCAACCACTGCAGCGACTA GGGACACTTGTAAATAGAAATCAGGAACATTTTTGCAGCTTGTTTCTGGAGTTGTTTGC GCATAAAGGAATGGTGGACTTTCACAAATATCTTTTTAAAAATCAAAACCAACAGCGATC TCAAGCTTAATCTCCTCTTCTCTCCAACTCTTTCCACTTTTGCATTTTCCTTCCCAATGCA GAGATCAGGGAAAAAAAAAAAAAAAAACCCAAACAAACAAAAGCACCCAGGCACCCAG TCTGAGTTCTGGGCAACTGATACGCCTGTTTCAGCAGCCTTTCTTTTTTTTCAATGAATG GGAATTGCAAATCAACTGGATTTTCATTATTTCCTTTTAATTTATATATGGAGAAATGTGA AGAGGGAAAGGAAATGGAAAGAGAAAGAGAAAGGGAGATAAAAATAGTGAAAATAAGA GCCTCCAGGCTCAGAAGAACTGATTACATTCTTAAGGTGAACAGGAAAAATACAATCTA TAACTTTCTTTGATGAGGAAAAATTAAGTTTACATTTTTCATATTTAGTGTTAAACAATTTA ATGTAGATTAAAATAAAAGACCAGTATTAGGAGGAAAAAACAAGTGCCTAAATGTCTTAA TGCTCTCTATGTGAGACAGAAATAGACGTGACCATTAGTAATGCAACTATTTTTGTCAAA TTTAGTGGGATTTTTTGGTTGTTGTTTGTTTTCTTGGGTTTTTTTTTTTTAAATGACAAACT CTAAAAATGTACCAATGTGAAAAAACACTTTCCTGAATGCCATTACTCATGCCCTCAAAG CTTTCATATCTGTAGCCTACTCCTGTAAAGGGTTTCTCCTGTTTCTAGTTTCTAGTTTGC AAAGGTATGCCAACGAATCTGGCAACCTGGTATTTGTTACTAAAACAGCATGTGTTTTC AGGTTTCTTTTCTATTGTACCTAAAGCAGTCTAAATTAAAACTTAGTAGAACACCAGGAG TATGATTCTGTTTCTGAAAGGTGAGTGGTGTATTGCTGTCATTGGGCCCTATTTTTTTTT TTAAATATATTTTTCTTTCTTACTTAATGGTGGCTGTGAATTGCAGGGTACTTTGAAGGC CATCATCTGAACCAAGAGTAGTAACTAGATTAATTATATGACAGAAAGAGTGAATTTAGC CTTGGGGTATTTATTAACTTCTATTATTTAGATATGCAATTTTGTTTACCACTATCTCTTC ACAGCATTCATATGTTAACTAAGCTCTTTTGTGTTAACAAGTTTATGACAAGACTGTGAA AGTAAAAATAATTTATCTGCTTGAAGACAAAAAAGGGAAGGAGAACAAGGATAGAAACA TTGTGAATTAATTTGTACAAATAGAAAGCAGACCAGCAGGACAGGAGCTCTTTTGCAGT GCTGCCGGATGGTGTCTAGAAAAATCCCAGTAATCATGTAGGCTCCATATTATTTTTGC CTGGGGCAAAATGATGTATCTTCTGTATTTAGCTTTTAAAATTAGTGAAACAAATGGCAT TATTTATTAAAATTCTACTCAGGATAACAGGATTGGCTTGCTTGTGCTTTGTAAAATATTG TTCCCCAGGGAGAATCTATTTATTTTCTGACATGACAGTTTCATAATTTTGATTTTTTCCC ATCTATAATTGTCACCATTAATATATTATTTCTTTCTTACTCTTTCCTATTTCTGTTCTGGT CTAGAAAACTGAGGTGTGCTTGGGGAAATCAAGGCCTTATTTTTTAAAACTGTCAAATA GCTTTGAGTTCCAGGGAGAAATGGTCACTGTACTTAACTGTACACACTTTTACTTATAAA GTCGTTTTTCCCCCTCAGTGATGGACAGATGTGCACACAGCCACTCAGAGACCATCATT TGGTTTGGGAGCTCAGGGTCCCAATCTCCCTTGTTAGTGTTTTGGATCATTAAGTCGGT GTTTATTTAAGTCACTAGGGTGTTTATTCTCCCTGTCTTGGGCCACGGAGAAATGCCAC CCTCTGCAGGAGGGGCTCTCACAGGGAATCTCCTCACATTCTTCACATTCACTCCCCAA AACAAACACCCTGCACAAAGATAGCTTTCAATGACCCTGACAGGCTTCAAAGGACGCT GCGGAAATCTCTCTGGAAAATAGGAAGGGCCAATTACTTTAATTTCTTACATGCCGTCT CTTCCTACTCCGGTGACTACCCTCCACTCTCCCAGCTCCGCCTGGGATAATTTTAGGCT CGTGGAAGTTGTGTCTCGCAGGGCGCCCCGGCCCACTTCCTGCTCCCTTAGTTGGTGA ATCTTGGAAATGTCTGCAGATCCGGCGAGGAGCAAGAGCGCTGGTCCTGTAAAAGCCC CCGTGATTACTGCTTGTAAACTTGTTAAAAGGACAATTTTCTGTCACAGTCATAAACTCT TTGTAAAACCCCTGGCACTAAATCCAGAGCGCGCATATTCCAGATGTGTTTAATAAGAA ATTGCACAATGTGCCTCCTTCCGCCATCCTCGGCTCAGTCTGGGTAAAAGGGGCGAGC AGGAGGAGGCGAGAAAGCTGGATATTGCTTTGAGTTTTTTTGGCAATCATTTCAGAGAG ATCATTAGGGGAAACTAACCATATTTTTTTCTTCCCTCGAGGAAATCTTCGAGAACCAGC TGTTAAGGGACCTCCAGTTTACCTTCTGGGCGTCGCAGTTTAACTGAATCTAAAGCCAC TTTTCGTTTCTCCTCTACAGATGTCATAACGGTAGCCAAAAGTAATGAACAACGGCTTAT AAATAGTTTGTAGGACGAAACCATACAGCATTGTGCCAAGTGAAATGAAAAAAAAAAAA AA. (SEQ ID NO:276; NM_005982.4), or a nucleic acid sequence that hybridizes to a nucleic acid sequence consisting of SEQ ID NO:276 under stringent hybridization conditions

In some embodiments, SIX homeobox 2 (SIX2) comprises the amino acid sequence: MSMLPTFGFTQEQVACVCEVLQQGGNIERLGRFLWSLPACEHLHKNESVLKAKAVVAFHR GNFRELYKILESHQFSPHNHAKLQQLWLKAHYIEAEKLRGRPLGAVGKYRVRRKFPLPRSI WDGEETSYCFKEKSRSVLREWYAHNPYPSPREKRELAEATGLTTTQVSNWFKNRRQRDR AAEAKERENNENSNSNSHNPLNGSGKSVLGSSEDEKTPSGTPDHSSSSPALLLSPPPPGL PSLHSLGHPPGPSAVPVPVPGGGGADPLQHHHGLQDSILNPMSANLVDLGS. (SEQ ID NO:277; NP_058628.3), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:277)

In some embodiments, the nucleic acid sequence encoding SIX2 comprises the nucleic acid sequence: AAGAAAGCTGAGAGCCAGCTAGACGGGAGGGAGAACGAGTGAGAAGCGAGCGAGGG AACGGCGGGCGGGCGCGGAGCATGCGGAGCGGCGCCCCGGGCGGCCCCCGGGCTT GGGCGAGGGCTTGGGCCAGGCGCGCGGGCCGTTGGGGTTCGGAGCTTCGTGGGACC CGCGGCCGGCGCGGGGACGTACGGCAGTGACTCGGGGCTCACCGGGGGCCAGTGC CGGGCCAGGGGGCCAGCCCCGCCCGCGTCTCGGCCCGGACGGCCCGGCGAGGAAG CTCCCATGCGGGACCGCGCGGCCCGGTGAGGGCGCGCGCGGGCGGGCGGGGACGC AGCCGGCACCATGTCCATGCTGCCCACCTTCGGCTTCACGCAGGAGCAAGTGGCGTG CGTGTGCGAGGTGCTGCAGCAGGGCGGCAACATCGAGCGGCTGGGCCGCTTCCTGT GGTCGCTGCCCGCCTGCGAGCACCTTCACAAGAATGAAAGCGTGCTCAAGGCCAAGG CCGTGGTGGCCTTCCACCGCGGCAACTTCCGCGAGCTCTACAAGATCCTGGAGAGCC ACCAGTTCTCGCCGCACAACCACGCCAAGCTGCAGCAGCTGTGGCTCAAGGCACACTA CATCGAGGCGGAGAAGCTGCGCGGCCGACCCCTGGGCGCCGTGGGCAAATACCGCG TGCGCCGCAAATTCCCGCTGCCGCGCTCCATCTGGGACGGCGAGGAGACCAGCTACT GCTTCAAGGAAAAGAGTCGCAGCGTGCTGCGCGAGTGGTACGCGCACAACCCCTACC CTTCACCCCGCGAGAAGCGTGAGCTGGCGGAGGCCACGGGCCTCACCACCACACAG GTCAGCAACTGGTTCAAGAACCGGCGGCAGCGCGACCGGGCGGCCGAGGCCAAGGA AAGGGAGAACAACGAGAACTCCAATTCTAACAGCCACAACCCGCTGAATGGCAGCGGC AAGTCGGTGTTAGGCAGCTCGGAGGATGAGAAGACTCCATCGGGGACGCCAGACCAC TCATCATCCAGCCCCGCACTGCTCCTCAGCCCGCCGCCCCCTGGGCTGCCGTCCCTG CACAGCCTGGGCCACCCTCCGGGCCCCAGCGCAGTGCCAGTGCCGGTGCCAGGCGG AGGTGGAGCGGACCCACTGCAACACCACCATGGCCTGCAGGACTCCATCCTCAACCC CATGTCAGCCAACCTCGTGGACCTGGGCTCCTAGAACCCATTTGCCTTGATGAGCTTG CCTTTTGTGACTTGACACTGGGGACGTGGAGTGGCGGTGTCCAGGGGCGCCCCGCCC CTGCGGCCCCACCAGGTACTGAAAGACCCGCAGGCTGAGCGGGTAGAACAGCCGGGT AGGGCAGATAGCTGTCTATGTTGGTTCTTGTTTGGGATTTATTTTCAACAAGTTACTTTT AGGATCCTTTTGGGGCTGGAGACTGAGTCTTGAACCACAGAAGGGAATAAATTATACAC CACTGTCATTCTCTCTCTCCCTCTGTCTCTTCCTTTTACCCTCTCTTGTCTTGCCTTTTCC CCCTTTCCTCTTCCTTTCCCTTCCTTCTCTTTTCTTTTTTCTGCTTTCTGTCTTTCTCCCT CTCCTTGTATTGCTTTCCTTCTAGATTTCTAGCTTGCCACCGTTCATTCTCTCCTTCTGT CTCTCCCTTTCTCTCTCCTTCTCTGTTTCTCCTCTCTTCTCTCCTGCCAGTCTCTTGTAC TCTGTGTCCTGGTCCCTCCGTATGTACCCCTGTCTTTCTCCTCCTGACTGGTGGTCTAT CTGCCCCTACCTCTGGCCCTCGCTTTACCGGAGTAGGGGGTGGGAGAGGGAAGAGGA GAGAAAATACAGGGACTTTGAACCTAGGCCATCTCCTGAGGCCTTTTCCCTCGCCCAT GTGGGTCAGTGGGAGCTGCAGGTGTCAGCTTTTCGTCTAGTAACTTAAGTGAGAGAGA AAGGGCAGCGCCACAGAAGCCCCTAAACGCCGCCTCGTCATACGCCCCTCCTCCTTCT CTCTTGGCGAGGCCCCGCCACACCGCGCTCTTCCTCCCGGGACTGTGACTACAGCGC TCCCGGCTGAGCGCGCCCCCCGAGCCGCCGACTTGCCGTCTCCCCGTAATGCCCTCA TGTGAATGTTCTTCGGGAAATATTTCTGCTTTTATTTTATAATAAAATTAGAAATCATAAA TATATAAATGGTTATATGCCACAA. (SEQ ID NO:278; NM_016932.5), or a nucleic acid sequence that hybridizes to a nucleic acid sequence consisting of SEQ ID NO:278 under stringent hybridization conditions

In some embodiments, EYA transcriptional coactivator and phosphatase 1 (EYA1, BOP, BOR, BOS1, OFC1) comprises the amino acid sequence: MEMQDLTSPHSRLSGSSESPSGPKLGNSHINSNSMTPNGTEVKTEPMSSSETASTTADGS LNNFSGSAIGSSSFSPRPTHQFSPPQIYPSNRPYPHILPTPSSQTMAAYGQTQFTTGMQQA TAYATYPQPGQPYGISSYGALWAGIKTEGGLSQSQSPGQTGFLSYGTSFSTPQPGQAPYS YQMQGSSFTTSSGIYTGNNSLTNSSGFNSSQQDYPSYPSFGQGQYAQYYNSSPYPAHYM TSSNTSPTTPSTNATYQLQEPPSGITSQAVTDPTAEYSTIHSPSTPIKDSDSDRLRRGSDGK SRGRGRRNNNPSPPPDSDLERVFIWDLDETIIVFHSLLTGSYANRYGRDPPTSVSLGLRME EMIFNLADTHLFFNDLEECDQVHIDDVSSDDNGQDLSTYNFGTDGFPAAATSANLCLATGV RGGVDWMRKLAFRYRRVKEIYNTYKNNVGGLLGPAKREAWLQLRAEIEALTDSWLTLALK ALSLIHSRTNCVNILVTTTQLIPALAKVLLYGLGIVFPIENIYSATKIGKESCFERIIQRFGRKVV YVVIGDGVEEEQGAKKHAMPFWRISSHSDLMALHHALELEYL. (SEQ ID NO:279; NP_000494.2), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:279)

In some embodiments, the nucleic acid sequence encoding EYA1 comprises the nucleic acid sequence: CTCAGATGCTATCTGCCGCTGCTGTTTGGTGGGGAAGGAGCGCTGGGCGCAAAGCTG TTACCAAACAGAACGGTGGGAGCTGATGGCTCCGAGTTTGGGGCGAGGTAGAAACTCT CCAGTGCCACTTCCGACTTTAAGCCTTCCTGTTGCCGTCCACTGTGGCGGGTTTCTTCC TGGGGAACACGTTTTCGCTCAGTCGCTCGGCAGCCCGAGCCTGCGGCAGCGGCCAGG CGCCTGCCCCCTGCGCCGAGCTTTCCCCTGCAGAGGCGCTCCACTCCCAGAAGCGCC GCGGCTGCACCAGAGCGCCTGAGAGCCCCCGCGCGTACCCATCCAGGAGCAAAACTA TGTCAGGAATGGAGGTTTGCTAACCCAGAAAATTCGAAGGAACACATTAAACTGGTGGA TGCAGCAGATGTAAGCGCTGTGCAAACATCTCAAGCCAGTTCAGATGTTGCTGTTTCCT CAAGTTGCAGGTCTATGGAAATGCAGGATCTAACCAGCCCGCATAGCCGTCTGAGTGG TAGTAGTGAATCCCCCAGTGGCCCCAAACTCGGTAACTCTCATATAAATAGTAATTCCA TGACTCCCAATGGCACCGAAGTTAAAACAGAGCCAATGAGCAGCAGTGAAACAGCTTC AACGACAGCCGACGGGTCTTTAAACAATTTCTCAGGTTCAGCAATTGGGAGCAGTAGTT TCAGCCCACGACCAACTCACCAGTTCTCTCCACCACAGATTTACCCTTCCAACAGACCA TACCCACATATTCTCCCTACCCCTTCCTCACAAACTATGGCTGCATATGGGCAAACACA GTTTACCACAGGAATGCAACAAGCTACAGCCTATGCCACGTACCCACAGCCAGGACAG CCGTACGGCATTTCCTCATATGGTGCATTGTGGGCAGGCATCAAGACTGAAGGTGGAT TGTCACAGTCTCAGTCACCTGGACAGACAGGATTTCTCAGCTATGGCACAAGCTTCAGT ACCCCTCAACCTGGACAGGCACCATACAGCTACCAGATGCAAGGTAGCAGTTTTACAA CATCATCAGGAATATATACAGGAAATAATTCACTCACAAATTCCTCTGGATTTAATAGTT CACAGCAGGACTATCCGTCTTATCCCAGTTTTGGCCAGGGTCAGTACGCACAGTATTAT AACAGCTCACCGTATCCAGCACATTATATGACCAGCAGCAACACCAGCCCAACGACAC CATCCACCAATGCCACTTACCAGCTTCAAGAACCGCCATCTGGCATCACCAGCCAAGC AGTTACAGATCCCACAGCAGAGTACAGCACAATCCACAGCCCATCAACACCCATTAAAG ATTCAGATTCTGATCGATTGCGTCGAGGTTCAGATGGGAAATCACGTGGACGGGGCCG AAGAAACAATAATCCTTCACCTCCCCCAGATTCTGATCTTGAGAGAGTGTTCATCTGGG ACTTGGATGAGACAATCATTGTTTTCCACTCCTTGCTTACTGGGTCCTACGCCAACAGA TATGGGAGGGATCCACCCACTTCAGTTTCCCTTGGACTGCGAATGGAAGAAATGATTTT CAACTTGGCAGACACACATTTATTTTTTAATGACTTAGAAGAATGTGACCAAGTCCATAT AGATGATGTTTCTTCAGATGATAACGGACAGGACCTAAGCACATATAACTTTGGAACAG ATGGCTTTCCTGCTGCAGCAACCAGTGCTAACTTATGTTTGGCAACTGGTGTACGGGG CGGTGTGGACTGGATGAGAAAGTTGGCCTTCCGCTACAGACGGGTAAAAGAGATCTAC AACACCTACAAAAATAATGTTGGAGGTCTGCTTGGTCCAGCTAAGAGGGAAGCCTGGC TGCAGTTGAGGGCCGAAATTGAAGCCCTGACCGACTCCTGGTTGACACTGGCCCTGAA AGCACTCTCGCTCATTCACTCCCGGACAAACTGTGTGAATATTTTAGTAACAACTACTCA GCTCATCCCAGCATTGGCGAAAGTCCTGCTGTATGGGTTAGGAATTGTATTTCCAATAG AAAATATTTACAGTGCAACTAAAATAGGAAAAGAAAGCTGTTTTGAGAGAATAATTCAAA GGTTTGGAAGAAAAGTGGTGTATGTTGTTATAGGAGATGGTGTAGAAGAAGAACAAGG AGCAAAAAAGCACGCGATGCCCTTCTGGAGGATCTCCAGCCACTCGGACCTCATGGCC CTGCACCATGCCTTGGAACTGGAGTACCTGTAACAGCGCTCGGCACTTTGACAGCGCA CAGCTGCTCTGTGACCAGGGACAGATCCAGCAGGCCCCAGTCTCGCATCAGCGCCGG CCTCCAGAACTTAGCAATTTCCGCCTGGTGATGCGCAGTTGCTGTCAGTCTTGACCTCT GCCTTTGTGGTGAATGGAGGACCACGTCTATTTCATCAGAACAGCTGTTGACTCTAGTA CTGTGAATCCAGTGAAAATAAGCCATGAGAATGTTTTAGCACAGCGTTATGTGTCTGCC ACATTAACTACACGGTTCAAACCTGTGAAGAAAGGACCTGCAAACGCTTCAGTTGTTAG CATTTTCAATGTGATATAAACAGCTTCTCCAATACAGCAAACCTAATTGCACAACAGAGA CTGAAATGTGTTTCCTGAATACCAGTGGAGGAATTTTCTTGTAAAGAAGGTTTACTTTTT GGTGTCTCATACCCAGGGTAATCTGTACATCTCTACTTATTTATGAACAGACTTTTTTTA AAAAGATAAAAAAACAGCTTTATTGAGGTATAATTCACCCACCAGACTTTTTTAAACATC AAATAATTGAAGAGACAATAGCATTAGAAATAAGTGATTAAAGGCCTCTGCCTCACAAC ATGGCAAGTACAGTACTTTGAATTTTAGCACATTGCATAGTAGTTTTAAGTATGTCTAAT TTAAACGTATAATATGTACATCACTGAGACAATCATGTACAGAAAGAATTTTTGGTGTAA ATTTGTAATAATGGATAATTCTTTTACATATTGTTTAGGGAAATGATATTGAAAGGTAGCA ATGCCTGGATAGTGAAGCATGAGGCAGCACGTGCACAAATTCATGTGCCGTGCCTTAT CTGAGTTTTCGGTATAAATATGTAGATAATGGATTTTTTTTTTTTAGATAATGTTGTCAAG ACCAAAAGCATGGATGTCAAGTGTCAGTAAGGATTTTGTTTTCTAAAATTTTTTCCTGCA TCAGTTCTTCTGAGGGCCTTGATGAAATAACACAGCAGTTTCTTAAACAATTTGAAACAA AATGAGCTCTCCTACCACCTCACTTTTTCATTTCCACACTAATGTATTATATGTAACTACT TGGAAAAAATAATTATTCAAATGCTTCTTCCCACAAAGAATATAGATGATAGTAGATATAT TTTATTAATAAAATGGTTCATGAATCGGAGACTAACAAAGTTTTCATGTGCTCAGAATTA TTAATTATCGTGTCTGCATTTTCTTTCGATAAAGGAAGACACACGATGCTAATCCGGAAA TCAGCAAACTTTGCATTACTCCCTATGTGCGTATTTTCTCTTTCTTCCTGTCACCCTGAG GAAGGTTCATTGCCATTGTCATCACCATGGAAACAACGTTCCTCTCCACCTGCATTATG TACTACATGACAGGCATCAATCTGGGGAAATAATAAAATTATCACCTTTGTCAGACCATA AGAGTTTCTCCAAAAGTGGTCAGTTTGGCTGGGCAATATTTTCTCTCATCTAACAAACAC AATCCATTGTCATGAAATTACCCTTAGGATGAGTCTTCTTTAATCAATCATATATTGGGC GGGAAAAACACCAGCTTTGACCCGAAGTAGTTGAAGAGCTACTTCATTCTTTTCTGAAG TTGTGTGTTGCTGCTAGAAATAGTCATTTGTGAATTATCCAAATTGTTTAAATTCACAATT GAATTAGTTTTTTCTTCCTTTTTGCTTGAAGCAAACAGTTGACAATTTTTAACCTTTTCAT TTTATGTTTTTGTACTCTGCAGACTGAAAAGACAAAGTTTATCTTGGCCTTACTGTATAA AGGTGTGCTGTGTCCACCGTTGTGTACAGAATTTTTCTTCATTAATTTTGTGTTTAAGTT AATAAAATTTATTTGTGATGTACTGTAA. (SEQ ID NO:280; NM_000503.6), or a nucleic acid sequence that hybridizes to a nucleic acid sequence consisting of SEQ ID NO:280 under stringent hybridization conditions

In some embodiments, EYA transcriptional coactivator and phosphatase 2 (EYA2, EAB1) comprises the amino acid sequence: MVELVISPSLTVNSDCLDKLKFNRADAAVWTLSDRQGITKSAPLRVSQLFSRSCPRVLPRQ PSTAMAAYGQTQYSAGIQQATPYTAYPPPAQAYGIPSYSIKTEDSLNHSPGQSGFLSYGSS FSTSPTGQSPYTYQMHGTTGFYQGGNGLGNAAGFGSVHQDYPSYPGFPQSQYPQYYGS SYNPPYVPASSICPSPLSTSTYVLQEASHNVPNQSSESLAGEYNTHNGPSTPAKEGDTDRP HRASDGKLRGRSKRSSDPSPAGDNEIERVFVWDLDETIIIFHSLLTGTFASRYGKDTTTSVRI GLMMEEMIFNLADTHLFFNDLEDCDQIHVDDVSSDDNGQDLSTYNFSADGFHSSAPGANL CLGSGVHGGVDWMRKLAFRYRRVKEMYNTYKNNVGGLIGTPKRETWLQLRAELEALTDL WLTHSLKALNLINSRPNCVNVLVTTTQLIPALAKVLLYGLGSVFPIENIYSATKTGKESCFERI MQRFGRKAVYVVIGDGVEEEQGAKKHNMPFWRISCHADLEALRHALELEYL. (SEQ ID NO:281; NP_005235.3), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:281)

In some embodiments, the nucleic acid sequence encoding EYA2 comprises the nucleic acid sequence: AGCAGCCGCGGCAGCCCAGGAGGCGGAGGCAGCGGCAACGGCAGAGACAGCAACGT GCCCGCCGCAGTCAGCCCGGCCTCGTCGGACCCGCACCGGCCCGCCCGCCCGCCCG CACCGCGTCGGGGCGCCCTCTCCACTGCGCGCGGTACAAGGAAATGGTAGAACTAGT GATCTCACCCAGCCTCACTGTAAACAGCGATTGTCTGGATAAACTGAAGTTTAACCGTG CTGACGCTGCTGTGTGGACTCTGAGTGACAGACAAGGCATCACCAAATCGGCCCCCCT GAGAGTGTCCCAGCTCTTCTCCAGATCTTGCCCACGTGTCCTCCCCCGCCAGCCTTCC ACAGCCATGGCAGCCTACGGCCAGACGCAGTACAGTGCGGGGATCCAGCAGGCTACC CCCTATACAGCTTACCCACCTCCAGCACAAGCCTATGGAATCCCTTCCTACAGCATCAA GACAGAAGACAGCTTGAACCATTCCCCTGGCCAGAGTGGATTCCTCAGCTATGGCTCC AGCTTCAGCACCTCACCCACTGGACAGAGCCCATACACCTACCAGATGCACGGCACAA CAGGGTTCTATCAAGGAGGAAATGGACTGGGCAACGCAGCCGGTTTCGGGAGTGTGC ACCAGGACTATCCTTCCTACCCCGGCTTCCCCCAGAGCCAGTACCCCCAGTATTACGG CTCATCCTACAACCCTCCCTACGTCCCGGCCAGCAGCATCTGCCCTTCGCCCCTCTCC ACGTCCACCTACGTCCTCCAGGAGGCATCTCACAACGTCCCCAACCAGAGTTCCGAGT CACTTGCTGGTGAATACAACACACACAATGGACCTTCCACACCAGCGAAAGAGGGAGA CACAGACAGGCCGCACCGGGCCTCCGACGGGAAGCTCCGAGGCCGGTCTAAGAGGA GCAGTGACCCGTCCCCGGCAGGGGACAATGAGATTGAGCGTGTGTTCGTGTGGGACT TGGATGAGACAATAATTATTTTTCACTCCTTACTCACGGGGACATTTGCATCCAGATACG GGAAGGACACCACGACGTCCGTGCGCATTGGCCTTATGATGGAAGAGATGATCTTCAA CCTTGCAGATACACATCTGTTCTTCAATGACCTGGAGGATTGTGACCAGATCCACGTTG ATGACGTCTCATCAGATGACAATGGCCAAGATTTAAGCACATACAACTTCTCCGCTGAC GGCTTCCACAGTTCGGCCCCAGGAGCCAACCTGTGCCTGGGCTCTGGCGTGCACGGC GGCGTGGACTGGATGAGGAAGCTGGCCTTCCGCTACCGGCGGGTGAAGGAGATGTAC AATACCTACAAGAACAACGTTGGTGGGTTGATAGGCACTCCCAAAAGGGAGACCTGGC TACAGCTCCGAGCTGAGCTGGAAGCTCTCACAGACCTCTGGCTGACCCACTCCCTGAA GGCACTAAACCTCATCAACTCCCGGCCCAACTGTGTCAATGTGCTGGTCACCACCACT CAACTAATTCCTGCCCTGGCCAAAGTCCTGCTATATGGCCTGGGGTCTGTGTTTCCTAT TGAGAACATCTACAGTGCAACCAAGACAGGGAAGGAGAGCTGCTTCGAGAGGATAATG CAGAGATTCGGCAGAAAAGCTGTCTACGTGGTGATCGGTGATGGTGTGGAAGAGGAG CAAGGAGCGAAAAAGCACAACATGCCTTTCTGGCGGATATCCTGCCACGCAGACCTGG AGGCACTGAGGCACGCCCTGGAGCTGGAGTATTTATAGCAGGATCAGCAGCATCTCCA CCTGCCATCTCACCCTCAGACCCCCTCGCCTTCCCCACCTCCCCACCGAGAACTCCAG AGACCCAGATGTTGGACACCAGGAAGGGGCCCCACAGCCGAGACGACGTGTCCAGTG ACCATCTCAGAAGCCGTCCATCAGTCCAAATGGGGGTTCTGAGAAGGAAAGTACCCAA CATTGGCTTCGGAGTATTTGACTTTGGGGAAAAGGGCTGGCTCGGAGTCTAGACTCTT CTGTAAGACTCACAGAACAAAAGCAAGGAATTGCTGATTTGGGGGGTGCCTGGTGATG AGGAGGGGATGGGTTTGTCTTGTCTTCTTTTTAATTTATGGACTAGTCTCATTACTCCGG AATTATGCTCTTGTACCTGTGTGGCTGGGTTTCTTAGTCGTTGGTTTGGTTTGGTTTTTT GAACTGGTATGTGGGGTGGTTCACAGTTCTAATGTAAGCACTCTATTCTCCAAGTTGTG CTTTGTGGGGACAATCATTCTTTGAACATTAGAGAGGAAGGCAGTTCAAGCTGTTGAAA AGACTATTGCTTATTTTTGTTTTTAAAGACCTACTTGACGTCATGTGGACAGTGCACGTG CCTTACGCTACATCTTGTTTTCTAGGAAGAGGGGGATGCTGGGAAGGAATGGGTGCTT TGTGATGGATAAAAGGCATTAAATAAAACCACGTTTACATTTTGAA (SEQ ID NO:282; NM_005244.5), or a nucleic acid sequence that hybridizes to a nucleic acid sequence consisting of SEQ ID NO:282 under stringent hybridization conditions

In some embodiments, ETS variant transcription factor 4 (PEA3, ETV4, E1AF, PEAS3) comprises the amino acid sequence: MERRMKAGYLDQQVPYTFSSKSPGNGSLREALIGPLGKLMDPGSLPPLDSEDLFQDLSHF QETWLAEAQVPDSDEQFVPDFHSENLAFHSPTTRIKKEPQSPRTDPALSCSRKPPLPYHH GEQCLYSSAYDPPRQIAIKSPAPGALGQSPLQPFPRAEQRNFLRSSGTSQPHPGHGYLGE HSSVFQQPLDICHSFTSQGGGREPLPAPYQHQLSEPCPPYPQQSFKQEYHDPLYEQAGQ PAVDQGGVNGHRYPGAGVVIKQEQTDFAYDSDVTGCASMYLHTEGFSGPSPGDGAMGY GYEKPLRPFPDDVCVVPEKFEGDIKQEGVGAFREGPPYQRRGALQLWQFLVALLDDPTNA HFIAWTGRGMEFKLIEPEEVARLWGIQKNRPAMNYDKLSRSLRYYYEKGIMQKVAGERYVY KFVCEPEALFSLAFPDNQRPALKAEFDRPVSEEDTVPLSHLDESPAYLPELAGPAQPFGPK GGYSY. (SEQ ID NO:283; NP_001073143.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:283)

In some embodiments, the nucleic acid sequence encoding PEA3 comprises the nucleic acid sequence: GCTCACAACTGTCTGCTGCGCCCGAAAAACAAGTCGGTGCGCTGGGGACCCGGGGCC GGGGCCGCCTTACTCCGGCCTAGCCCCGCGGCCCTCGGTGCGGGCTCCAGGGCATG CTCGGGACCCCCCGCGGCTCCAGCCCAGACGCCCCGGCCTCAGGTCTCGGCCCCCG CTTGGGGCCCCGGCCGTGCGGCCGGAGGGAGCGGCCGGATGGAGCGGAGGATGAA AGCCGGATACTTGGACCAGCAAGTGCCCTACACCTTCAGCAGCAAATCGCCCGGAAAT GGGAGCTTGCGCGAAGCGCTGATCGGCCCGCTGGGGAAGCTCATGGACCCGGGCTC CCTGCCGCCCCTCGACTCTGAAGATCTCTTCCAGGATCTAAGTCACTTCCAGGAGACG TGGCTCGCTGAAGCTCAGGTACCAGACAGTGATGAGCAGTTTGTTCCTGATTTCCATTC AGAAAACCTAGCTTTCCACAGCCCCACCACCAGGATCAAGAAGGAGCCCCAGAGTCCC CGCACAGACCCGGCCCTGTCCTGCAGCAGGAAGCCGCCACTCCCCTACCACCATGGC GAGCAGTGCCTTTACTCCAGTGCCTATGACCCCCCCAGACAAATCGCCATCAAGTCCC CTGCCCCTGGTGCCCTTGGACAGTCGCCCCTACAGCCCTTTCCCCGGGCAGAGCAAC GGAATTTCCTGAGATCCTCTGGCACCTCCCAGCCCCACCCTGGCCATGGGTACCTCGG GGAACATAGCTCCGTCTTCCAGCAGCCCCTGGACATTTGCCACTCCTTCACATCTCAG GGAGGGGGCCGGGAACCCCTCCCAGCCCCCTACCAACACCAGCTGTCGGAGCCCTG CCCACCCTATCCCCAGCAGAGCTTTAAGCAAGAATACCATGATCCCCTGTATGAACAG GCGGGCCAGCCAGCCGTGGACCAGGGTGGGGTCAATGGGCACAGGTACCCAGGGGC GGGGGTGGTGATCAAACAGGAACAGACGGACTTCGCCTACGACTCAGATGTCACCGG GTGCGCATCAATGTACCTCCACACAGAGGGCTTCTCTGGGCCCTCTCCAGGTGACGG GGCCATGGGCTATGGCTATGAGAAACCTCTGCGACCATTCCCAGATGATGTCTGCGTT GTCCCTGAGAAATTTGAAGGAGACATCAAGCAGGAAGGGGTCGGTGCATTTCGAGAGG GGCCGCCCTACCAGCGCCGGGGTGCCCTGCAGCTGTGGCAATTTCTGGTGGCCTTGC TGGATGACCCAACAAATGCCCATTTCATTGCCTGGACGGGCCGGGGAATGGAGTTCAA GCTCATTGAGCCTGAGGAGGTCGCCAGGCTCTGGGGCATCCAGAAGAACCGGCCAGC CATGAATTACGACAAGCTGAGCCGCTCGCTCCGATACTATTATGAGAAAGGCATCATGC AGAAGGTGGCTGGTGAGCGTTACGTGTACAAGTTTGTGTGTGAGCCCGAGGCCCTCTT CTCTTTGGCCTTCCCGGACAATCAGCGTCCAGCTCTCAAGGCTGAGTTTGACCGGCCT GTCAGTGAGGAGGACACAGTCCCTTTGTCCCACTTGGATGAGAGCCCCGCCTACCTCC CAGAGCTGGCTGGCCCCGCCCAGCCATTTGGCCCCAAGGGTGGCTACTCTTACTAGC CCCCAGCGGCTGTTCCCCCTGCCGCAGGTGGGTGCTGCCCTGTGTACATATAAATGAA TCTGGTGTTGGGGAAACCTTCATCTGAAACCCACAGATGTCTCTGGGGCAGATCCCCA CTGTCCTACCAGTTGCCCTAGCCCAGACTCTGAGCTGCTCACCGGAGTCATTGGGAAG GAAAAGTGGAGAAATGGCAAGTCTAGAGTCTCAGAAACTCCCCTGGGGGTTTCACCTG GGCCCTGGAGGAATTCAGCTCAGCTTCTTCCTAGGTCCAAGCCCCCCACACCTTTTCC CCAACCACAGAGAACAAGAGTTTGTTCTGTTCTGGGGGACAGAGAAGGCGCTTCCCAA CTTCATACTGGCAGGAGGGTGAGGAGGTTCACTGAGCTCCCCAGATCTCCCACTGCG GGGAGACAGAAGCCTGGACTCTGCCCCACGCTGTGGCCCTGGAGGGTCCCGGTTTGT CAGTTCTTGGTGCTCTGTGTTCCCAGAGGCAGGCGGAGGTTGAAGAAAGGAACCTGG GATGAGGGGTGCTGGGTATAAGCAGAGAGGGATGGGTTCCTGCTCCAAGGGACCCTT TGCCTTTCTTCTGCCCTTTCCTAGGCCCAGGCCTGGGTTTGTACTTCCACCTCCACCAC ATCTGCCAGACCTTAATAAAGGCCCCCACTTCTCCCA. (SEQ ID NO:284; NM_001079675.5), or a nucleic acid sequence that hybridizes to a nucleic acid sequence consisting of SEQ ID NO:284 under stringent hybridization conditions

In some embodiments, nuclear factor of activated T cells 5 (NFAT5, NFATZ, OREBP, NF-AT5, NFATL1, TONEBP) comprises the amino acid sequence: MPSDFISLLSADLDLESPKSLYSRDSLKLHPSQNFHRAGLLEESVYDLLPKELQLPPSRETS VASMSQTSGGEAGSPPPAVVAADASSAPSSSSMGGACSSFTTSSSPTIYSTSVTDSKAMQ VESCSSAVGVSNRGVSEKQLTSNTVQQHPSTPKRHTVLYISPPPEDLLDNSRMSCQDEGC GLESEQSCSMWMEDSPSNFSNMSTSSYNDNTEVPRKSRKRNPKQRPGVKRRDCEESNM DIFDADSAKAPHYVLSQLTTDNKGNSKAGNGTLENQKGTGVKKSPMLCGQYPVKSEGKEL KIVVQPETQHRARYLTEGSRGSVKDRTQQGFPTVKLEGHNEPVVLQVFVGNDSGRVKPH GFYQACRVTGRNTTPCKEVDIEGTTVIEVGLDPSNNMTLAVDCVGILKLRNADVEARIGIAG SKKKSTRARLVFRVNIMRKDGSTLTLQTPSSPILCTQPAGVPEILKKSLHSCSVKGEEEVFLI GKNFLKGTKVIFQENVSDENSWKSEAEIDMELFHQNHLIVKVPPYHDQHITLPVSVGIYVVT NAGRSHDVQPFTYTPDPAAGALNVNVKKEISSPARPCSFEEAMKAMKTTGCNLDKVNIIPN ALMTPLIPSSMIKSEDVTPMEVTAEKRSSTIFKTTKSVGSTQQTLENISNIAGNGSFSSPSSS HLPSENEKQQQIQPKAYNPETLTTIQTQDISQPGTFPAVSASSQLPNSDALLQQATQFQTR ETQSREILQSDGTVVNLSQLTEASQQQQQSPLQEQAQTLQQQISSNIFPSPNSVSQLQNTI QQLQAGSFTGSTASGSSGSVDLVQQVLEAQQQLSSVLFSAPDGNENVQEQLSADIFQQVS QIQSGVSPGMFSSTEPTVHTRPDNLLPGRAESVHPQSENTLSNQQQQQQQQQQVMESSA AMVMEMQQSICQAAAQIQSELFPSTASANGNLQQSPVYQQTSHMMSALSTNEDMQMQCE LFSSPPAVSGNETSTTTTQQVATPGTTMFQTSSSGDGEETGTQAKQIQNSVFQTMVQMQ HSGDNQPQVNLFSSTKSMMSVQNSGTQQQGNGLFQQGNEMMSLQSGNFLQQSSHSQA QLFHPQNPIADAQNLSQETQGSLFHSPNPIVHSQTSTTSSEQMQPPMFHSQSTIAVLQGSS VPQDQQSTNIFLSQSPMNNLQTNTVAQEAFFAAPNSISPLQSTSNSEQQAAFQQQAPISHI QTPMLSQEQAQPPQQGLFQPQVALGSLPPNPMPQSQQGTMFQSQHSIVAMQSNSPSQE QQQQQQQQQQQQQQQQQSILFSNQNTMATMASPKQPPPNMIFNPNQNPMANQEQQNQ SIFHQQSNMAPMNQEQQPMQFQSQSTVSSLQNPGPTQSESSQTPLFHSSPQIQLVQGSP SSQEQQVTLFLSPASMSALQTSINQQDMQQSPLYSPQNNMPGIQGATSSPQPQATLFHNT AGGTMNQLQNSPGSSQQTSGMFLFGIQNNCSQLLTSGPATLPDQLMAISQPGQPQNEGQ PPVTTLLSQQMPENSPLASSINTNQNIEKIDLLVSLQNQGNNLTGSF. (SEQ ID NO:285; NP_001106649.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:285)

In some embodiments, the nucleic acid sequence encoding NFAT5 comprises the nucleic acid sequence: AGATTCCTGTCAGCGGCGGCGGCGGTGGCGGCGACCGTCAGTTTTCGCTGAGGAGAA ACACGAAACGGACCCTTTGGCTCTCCCCCTTCCCCTTCCCCGTCCTGAACCCCTCTCC TGGTCACCGAGAATCAGTCCCCGTGGAGTTCCCCCTCCACCTCGCCATCGTTTCCTCG GTCCTCGGCCCAGTGGAAGTCACTACCCTCGAGGAGGAGGCAGCGGCAGCCGCCCTC GCGTCGCCGCCCCCGGTTCGGTGCCCGCGGTCCCGGAGAGGAGGTGCCGCCGCCAC CGCCGCTCCCCCCCTCCCGCTGCCCTCGGGCCGGGCTGGGTCGAGCTGCGATGCCC TCGGACTTCATCTCATTGCTCAGCGCGGACCTAGACCTGGAATCGCCCAAGTCCCTCT ACTCGCGAGATTCTCTGAAGTTACACCCATCACAGAATTTTCATAGAGCTGGACTATTG GAAGAATCTGTCTATGATCTTCTCCCAAAGGAGTTACAGTTACCTCCATCTAGAGAAAC ATCTGTAGCATCAATGAGTCAGACAAGCGGTGGTGAGGCAGGCTCGCCTCCTCCAGCT GTTGTTGCTGCTGATGCTTCTTCAGCTCCCTCCTCTTCCTCCATGGGCGGTGCTTGCAG CTCCTTTACCACCTCTTCCAGCCCTACCATTTATTCTACCTCAGTCACCGACAGCAAGG CTATGCAAGTGGAGAGCTGCTCCTCAGCCGTGGGGGTAAGTAACAGAGGGGTAAGTG AAAAGCAGTTAACCAGTAACACAGTTCAGCAGCATCCATCAACACCGAAGAGGCACAC AGTCTTGTACATCTCACCACCACCTGAGGACTTGCTGGATAACAGTCGGATGTCCTGC CAGGATGAGGGGTGTGGATTGGAATCTGAGCAGAGCTGCAGTATGTGGATGGAGGAT TCCCCCTCCAACTTCAGTAACATGAGCACCAGTTCCTACAATGATAACACTGAGGTACC TCGTAAATCACGAAAACGAAATCCAAAGCAGAGGCCGGGGGTCAAACGACGAGATTGT GAAGAATCTAATATGGATATATTTGATGCCGACAGTGCCAAAGCACCTCACTATGTGCT TTCTCAGCTTACCACGGACAACAAAGGCAACTCAAAAGCGGGAAATGGAACATTGGAA AACCAAAAAGGAACTGGAGTAAAGAAGAGCCCTATGTTGTGTGGACAATATCCTGTTAA AAGTGAGGGAAAGGAGCTGAAGATAGTTGTACAACCTGAGACACAGCACCGAGCTCG GTACCTGACTGAGGGCAGCCGTGGCTCAGTGAAAGATAGAACACAGCAAGGCTTTCCT ACAGTAAAGCTGGAAGGCCATAATGAACCTGTAGTGTTGCAAGTGTTTGTGGGCAACG ACTCTGGACGAGTGAAACCACATGGATTTTATCAGGCCTGCAGAGTAACTGGACGAAA TACAACTCCTTGCAAAGAAGTGGACATTGAAGGCACTACTGTTATAGAAGTCGGCCTTG ATCCTAGCAACAACATGACACTGGCGGTGGACTGCGTAGGGATATTGAAATTGAGGAA TGCTGATGTCGAAGCCAGAATAGGAATTGCTGGTTCCAAGAAGAAAAGCACTCGTGCC AGATTGGTTTTTCGAGTTAATATCATGAGGAAAGATGGCTCCACTTTGACACTGCAAAC ACCCTCTTCTCCAATTTTGTGTACTCAGCCAGCAGGAGTGCCAGAAATCTTAAAGAAAA GCTTGCATAGCTGTTCAGTGAAAGGAGAAGAAGAAGTGTTTTTAATCGGCAAGAACTTT CTGAAAGGAACTAAAGTTATTTTCCAAGAAAATGTTTCTGATGAAAACTCTTGGAAGTCA GAAGCTGAAATTGATATGGAACTATTTCATCAGAATCATCTTATTGTGAAGGTTCCTCCC TATCATGACCAACATATAACTTTGCCTGTGTCAGTGGGAATATATGTAGTGACAAATGCT GGAAGATCTCATGATGTTCAACCATTCACTTACACTCCAGACCCAGCAGCTGGTGCTTT GAATGTAAATGTGAAGAAGGAAATATCTAGTCCAGCAAGACCTTGCTCTTTTGAAGAGG CCATGAAAGCAATGAAAACTACTGGATGTAATTTAGATAAGGTAAATATTATCCCTAATG CCCTGATGACTCCACTCATACCAAGCAGTATGATTAAGAGTGAAGATGTTACTCCAATG GAAGTAACAGCAGAAAAAAGATCTTCCACTATTTTTAAGACTACAAAGTCTGTTGGATCA ACTCAGCAAACATTAGAAAACATCTCAAACATAGCAGGAAATGGCTCTTTTTCATCACCA TCATCTTCCCACCTACCTTCTGAAAATGAAAAACAGCAGCAGATTCAGCCCAAGGCATA CAACCCAGAGACCCTGACAACTATTCAAACCCAGGACATCTCACAGCCTGGTACTTTTC CAGCAGTTTCTGCTTCTAGTCAGCTGCCCAACAGCGATGCACTATTGCAGCAGGCTAC ACAGTTTCAGACAAGAGAAACTCAGTCTAGAGAGATATTACAGTCAGATGGTACAGTGG TTAATTTGTCACAACTGACTGAGGCATCACAACAACAGCAGCAGTCACCACTACAAGAA CAAGCACAGACTTTACAGCAGCAGATTTCATCAAATATTTTTCCATCACCAAATAGTGTG AGTCAGCTTCAGAATACTATTCAGCAGCTGCAAGCAGGGAGTTTCACAGGCAGTACTG CTAGTGGCAGCAGTGGAAGTGTTGACTTGGTCCAACAAGTTTTAGAGGCACAGCAGCA GTTATCTTCAGTTTTATTTTCTGCTCCAGATGGTAATGAGAATGTTCAAGAGCAGCTTAG TGCAGATATTTTTCAACAAGTCAGTCAAATTCAGAGTGGTGTAAGCCCTGGAATGTTTTC CTCAACAGAGCCAACAGTCCATACCAGACCAGATAATTTATTACCTGGAAGAGCTGAAA GTGTTCATCCACAGTCTGAAAACACGTTATCTAATCAACAGCAGCAGCAGCAGCAGCAA CAGCAAGTGATGGAATCTTCAGCCGCAATGGTGATGGAGATGCAACAGAGTATCTGCC AGGCAGCTGCCCAGATTCAGTCAGAGTTATTCCCTTCAACTGCTTCAGCAAATGGAAAC CTTCAGCAATCGCCAGTTTACCAGCAGACTTCTCACATGATGAGTGCATTGTCTACCAA TGAGGATATGCAAATGCAGTGTGAATTGTTTTCTTCTCCTCCTGCAGTTTCTGGAAATGA AACTTCTACAACTACCACACAGCAGGTTGCAACCCCTGGCACTACCATGTTTCAGACAT CAAGTTCAGGAGATGGAGAAGAAACTGGAACACAAGCAAAACAGATTCAGAACAGTGT CTTTCAGACCATGGTCCAAATGCAACATAGTGGGGACAATCAACCTCAAGTTAACCTTT TTTCATCCACAAAAAGTATGATGAGTGTTCAGAATAGTGGTACCCAACAACAAGGTAAT GGTTTATTCCAGCAAGGGAATGAGATGATGTCACTTCAATCTGGAAATTTTTTGCAGCA GTCTTCTCATTCACAGGCCCAACTTTTTCATCCTCAAAATCCTATTGCCGATGCTCAGAA CCTTTCCCAGGAAACTCAAGGTTCTCTCTTTCATAGTCCAAATCCTATTGTCCACAGTCA GACTTCTACAACCTCCTCTGAACAAATGCAGCCTCCAATGTTTCACTCTCAAAGTACCAT TGCTGTGTTACAGGGCTCTTCAGTTCCTCAAGACCAGCAGTCAACCAACATATTTCTTT CCCAGAGTCCCATGAATAATCTTCAGACTAACACAGTAGCCCAAGAAGCATTTTTTGCA GCACCGAACTCAATTTCTCCACTTCAGTCAACATCAAACAGTGAACAACAAGCTGCTTT CCAACAGCAAGCTCCAATATCACACATCCAGACTCCTATGCTTTCCCAAGAACAGGCAC AACCCCCGCAGCAGGGTTTATTTCAGCCTCAGGTGGCCCTGGGCTCCCTTCCACCTAA TCCAATGCCTCAAAGCCAACAAGGAACCATGTTCCAGTCACAGCACTCAATAGTTGCCA TGCAGAGTAACTCTCCATCCCAGGAACAGCAGCAGCAGCAGCAACAGCAGCAGCAACA GCAGCAGCAACAACAACAGAGCATTTTATTCAGTAATCAGAATACCATGGCTACAATGG CGTCTCCAAAGCAACCACCACCAAACATGATATTCAACCCAAATCAAAATCCAATGGCT AATCAGGAGCAACAGAACCAGTCAATTTTTCACCAACAAAGTAACATGGCCCCAATGAA TCAAGAGCAACAGCCCATGCAATTTCAGAGTCAGTCCACAGTTTCCTCACTTCAGAACC CAGGTCCTACCCAGTCGGAATCATCACAGACCCCCTTGTTCCATAGCTCTCCTCAGATT CAGTTGGTACAAGGGTCACCTAGTTCTCAAGAGCAGCAAGTAACTCTCTTCTTATCTCC AGCATCCATGTCTGCCTTGCAGACCAGTATAAATCAACAAGATATGCAACAGTCTCCTC TTTATTCCCCTCAGAACAACATGCCTGGAATTCAAGGAGCCACATCTTCGCCTCAACCA CAGGCTACTTTATTTCACAACACAGCAGGAGGCACAATGAACCAACTGCAGAATTCTCC TGGCTCATCTCAGCAGACATCAGGAATGTTCTTATTTGGCATTCAAAATAACTGTAGTCA GCTTTTAACCTCTGGACCAGCTACATTGCCTGATCAGTTGATGGCCATAAGTCAGCCAG GCCAACCACAAAACGAGGGCCAGCCACCTGTGACAACACTTCTTTCTCAGCAAATGCC AGAGAATTCTCCACTGGCATCCTCTATAAACACCAACCAGAACATCGAAAAGATTGATT TGCTTGTTTCATTGCAAAACCAAGGGAACAACTTGACTGGCTCCTTTTAACTGGATATAA ATTCCACGAAGAAAATCCTGATTCCAAGATGTCCTGAGATCTTGTGGTTCCATGAGAAT TATTACTTTAAAAACAAAACAAAATATAAAAAACTGTGTTTGAGTAAACTGATAGATTTTA CTCTGACTGCAAAAGAGCACACCTATGCTGCTTGTTGCAGTAACTAACCACCAATGTTA ACATCTTCATATTTTATATTCCTAATAACAGTGATGACTGAGAATCTATTTGAGTTTCCAG CTGGCAGAATTAATTGTTATTATTTTCCTAGGCGCAATTTCCTTAAACGTACAGTTTAAAT TCAAGGCTGGACCACTCAGTTATTATTGCTATTAGAAAATAATATATCATGTTTACTTTTG TTCTTCATTATTTTCTTTCCTGCATTGTTTTAGTCAAGTAATGGCTTTTGAAAAAGTAAAG TTCAATAATAACTAAGGCTGTGATTTTTTTCAATATAAAAGGCACAGCTGTTGGCCAAAG TGAAGGAATCTTTTTTCAGTTTTATTGGAGAAACTGAAGGGTAACATTCTAACAAGTAAA CTGTATGTGCAGATAAAAGTACTCTTGATTTAACACAAAGGCAGATGATACACTTATAAA ACTGGGAACAGCTGGAATGCTTCTTGATTTTATTTTTTCAGAGAGTTGTTAGTTCTCTGG GTTTCTACTAAGGGGTTTAGCCATAACTGTGCATAGAAAAATAATTATCTGTAAAAAATG AAGGGGATAATATATGATAAATTATGTTCTGATATCCTCCTACAGTAGTTTAAATTGACA GAAAAATTTGAATGTTTTCTTCTTAACCCAGTCTTAGGCTGGTATTCCCTTTTTATATATA TCTATATTACTTTTCACCTCTTTTTCACTTTACTTTAGAGAACTATTAATATACTACTGGCT TCATGACCCTGTAGCATCTTTGGCCACTTTAATCTAGGGTGACCTAGCAATCCTGCAGC ACAGGGCAGAGAGTACTGTCTTAGGAATTATTAGGAGTTGATTCCTGAGAAACAACACA TTTTTCCCCATGAACGGTGCTGTTCTGAAGTCTTCAAATTTTTCCCTCTAATAGGAAACA GTATAAATTTTAATTAAAAAAAAAAGGCAAACTAAAATTTCTTGAAATATCACTTCTCCCT GATCTGCAGTGAGTATAAATTCACTTGTCACCTCAGTGCTTTACAGTTTGAAGTGGTCA CTTACCTGATGGTTCCCACAAGCCTTAGGCTTTACAGGGTTGTATCATTGACTTAAAAT GAAGAATTAACTTGTGTTACATCTATAAAGAGCAAAATAACACACTCCAGAACTTGGCA GTTGTAGCATTAGTTATACAGTTTTGGGTGTTCTTGCCACCCGTGGGATGCCTGCTTCT CACTACCACCTGTGTCTGGACACATGCTTATGTCTCATTTTCCTTTTGGCATGTGGAAA GCTGTCAATGCAGTGTAAGGCCAACGTGTGTGTGGCTTCTATGTGTTGAGATAATGTTT TGGTATCCTTGTCCGTTTCATTTATTTTTTAAGTGTACAAAAAATAACCTGTTAATTGTTG AAGGCTACTTTTCTGTTCTTTTTTTTTTTTTTTTTCTATCCTGTACATTTAGTTGAACTGTG CGGAATTGTGGTGTTGGTTTTGTTTACACAGCCAGATTTTTCCTTCTTTTTGTTTTGTGA TGATCTTCCTTTGTTCTTTGAATGTGCTCTTTTGTCTTTTTCTCTTTTTTCTCATGTTTTCT TCCCTCCACCTCCACCCCTTTCTTTCTTTCTCTCTCTGATTGAGAGGCATTGAATTACGT TTTCAGTAGTACAGGCTTCTTGCCGATATGAAGGGAACTTTTCAGAAAGAGACCTACTC TGGGTCATTTAATTTTGAATACAGTTTTCAATCGTTCAAGTTTTGGATGGTTTATATCTAA TGTGTGTTTCATTTTTTTGGAAAGCTATATTTTGTATTTAGGAAATGGTATACTATTTTGC TATTTGTACTGAGTGAGTACATTGGCATAAATATAGAAATTTATATATATACATATATATA AACTATTCTTTTTTGCCACACATTTTTGTGGTAAATTTGTGAGTTTGTCTGATGTTCTACC ACAACGTGGCGTCTGATAACAGTGAGGGGGGGTGGGGTTTGTTATGTCTTTATTGAGT ATTTAAGTATCTTTTGAAACAAATGACCTGTTCATCTGTGGCCATTCCATCAGGCAGTTA GTTCCTTGATGTCAGTAGTGGGCTAAAGGCAGCTTACTGTGTGTTTGCTGGAGCTTTCA CTCAGCCAAGTGTTAGAGTCAGGAAACCCATTGAGGCAATGGCGTCAAATGGTGTTTC ACAAGAATGAGCCATTCAGTCTTTGCTCACTATATATTTAATATTTTATTATTGTTGTTAT TGTTATTATTAATTGGCTTTCTGTATTCTATGCCTTTTATTTATAAAGACACTAAGAAAAC CCATGTTTGTAATTTTAATAACATTTTTCCCATCTTGTAATATCCAGAGCTACTTTATAAA TTCTCTGAACCAAAAGTATTTTCCTCAGTGTATCTCTTCTCCCCCAGCCCCTATTGGGAA AAATTACCCAGTATAGTTCAGGTTATGAGGAGGATCAGCCACACAATCCAGTGCTTCAG TTTGAAAATGTAAAATTCTAACCCTAAAGTAGGGTTGGTTGAAATTTCAGACAAAGCAAA CCCAGCAGGTATAAAAAGTAGTATAAATACAAATCTGTAAGTTATTTTTGAATTTTCTGAA CTTTTTTCTAAGAGATTACATAGGAGACTAAAGAAATCTATCTGTTCAAGTTCTAATTAG GATGATTGTTAATACTGCACTGTGGATGAAGTGGCGACTGGCTTGTGTGCTGACTTCTG TGGTTTAGCAAGAGGTTTATTGTTATCAAATGCTAATTGGCAATGCCAAGTCACTGGGA CCAATTTTCTGTTTTATAATATCTAAGTTTAGAACAGAATATATACCTGAACTGTAGTGGT TTGATCGGATGGAGACAGAAAACCCGATTTTTATTCTCATAAATTTTGTGGTTATTTATA CAAGGGCTGTGCTATGCTACCATATTCTTGTTCAATAATAATAGGTTTGTTGTTTTTTTTA CATTGTTAAATGTTCCTTACCCCTAAAGGTCAATGTTAAGTACAACATTCTGAAAATACA ATTTGGCTACGAAGAGTATTCATCTTCTTTGAAGCTCAGTGGTTGATATTTGTGCTAATA ATGCAATTTCCTGATTACTGTTACAAGTTATAGCTACATATGGGAGAGACTCAGTGAGC CAGCAAAGGCCATAGAAACAACAATTTATTAAATGTATTTATGGCAGAAGGACCTAAATA AACTGTGAGCCACCTTTTCTTCTTTATATTGTTACATTTAAGTGTTCTTGCTTTCAGCAAC TCACATTAATGCTTGGAGCTTATCTCTTTCTCTCTCTCTCTCTCTCTCTCTGTGTGTGTG TGTGTATGTGTGTGTGTGTGTGTGTGTGTTTCCTTATTGTCATTCCATTATATATCCACA CCAACATGGGTGACGATAATTCAAAGTCATATTTTGCCTCTAAGCTTGATCATGTTACCT TTATGATTAAAGTATCATGTTATTTAGCCAATGCAAATCTGTTTTAAAACAAATAGTTTAA AAAAAGAACAAGTTTTTAAGGGCTTTATTATAGAAGAAGTATTAATGAAGGACTTTCCTT CCTCCCTCCCTTTCCTCCCCTCCCTGCCTCCCTTCTTCCCTTCCATCTCCCCCTCCTCC CTGCCTTCTTTGTTTCTCCTTCCCTTATTCCTCCCTCCCTCCTTTCTCCCTTCCTTCCTTT CTTCCATTCATCCTTCCTTGCCTTTTATTTTTATTTTTTGTAATATCACATGTGCTGTAGTT TGGAATTTTATTCTAGTGCATTTCTTGCTCATCAGAACCTCAGCTAATCTACCTAGGAAA AATAGTATCAAAGGAAATGAGAAAGTTGTATCTGAGTCCCTCCAGAACTAAGATAATTCT TTTTGACCATTTAAGCCTTTATAAATGCGTTTTGACCATTTAAGCCTTTATAAATGCTTGT TTTAGGAAAGTGAATCTGTTAGATGCATCAACAAATAATGACCAGGACAAAACGATTTAA TAATTAAAGTCTCAAATCACCATGGTTATACATTTTCACCAGAAATAGTAATCTTACAATT TTTCATTTTTCTGATGAAGATTTCTGTTCCAATATCTGTTTCCTAATAGATTTTTTAAATTA ATTAGCTTTCCTCTGCTTTATGACCACAGGTTTTATCCCTAACCGAGACAGCTGTCTTAT ATCTGCATGCCTTAGACTGTGTGGAGGGACTCCATGAAGAAAGACCATAGGTTAGAAA AATAACTCATAGTATATACCCTAGTAAGTGGGTTAGTAGAATCTCATAACATGTATTAAA AAGAGGTTTTCTTCTCTGCTTGTTTGTGTCACTAGAGCAAAATTGTAGAGATAATGCTCA TAATGCAGTAAATATCAGAATAATATCTACAATATCATTTGTGGATGGTCCCAGGTCCCA GTGCTCTAGTTACTTTACTTCTTTTTTTTTTTTTGAGATGGAGTCTTGCTCTGTCTCTCAG GCTAGAGCAGTGTGCGATCTCAGCTCACTGCAGCCTCCACCTCCCAGGTTCAAGCGAT TCTCCTGCCTCAGCCTCCCAAGTAGCCAGGATTACAGGCACCCTCCACTAGGCCCGGC TAATTTTTTTTGTATTTTTTTAGTAGAGATGGGGTTTTGCCATGTTGGCCAGGCTGGTTT CGAACTCCTAACCTCCAGTGATCCACCTGCCTCGGCGTCCCAAAGTGCTAGGATTACA GGCATGAGCCACCACATCCGGCCTAATTACTTCTTTAATCCCCATTTATTTTTATGCCAT TCTAGCCTCATTTATTAATAAAATTATGTTTTTACTTTCTCTTTCAGGAAATTTTTTAAATT AATATTTTATATCTAGATCTAATGCTATGGAAAAGTGCCTTTTTATCATTTATAATTTCATT TTTCACTATTTCCAAAAACACATAAACAAATAGTTTCAGTAGGTCCCAGCTTTTACTTTTT CCATTTAAACCTTCTTTTCTCCATTTCTTCCCTTTGGCTTAAGAATAAAAGAAAAGGTACA TTGCTAGAATTGTTTCTTTGGGAGAGGGTAAAAGATTACAGAATTAGACTGTTCAGCCTT TATATAAACTAAATTTGTCTTCATCTCAACCAGCTAATGGTAGGTCTTATCTGAATACTCA TGAGAATTTTAGCATCTGTGAAACTCCATGCACCAGATGTGTGTAAATTTCAGGAAGAA AGTGTTGAAAGCATTTTCTCTGATGTTAATTAGATGGAAATAAATCACTAAAACATAGTTT AGGTAAAGCCTGATTATGCCACTTTTTTTTAACTAGACAGGGCAAAGTTGTTTATGTTAG TGTACTTCTTGTCTATCCTCAGTTAATTTACCTAGACAAAAAGTGTCAAAGGAAATGAGA AAAAGGTTATATCTGACTCCCTCCAGACCTAAGATAATTCCTTTGATCAGATACAGTCAG ATGGAGTGCCTTGGTTTTTGTTAATTTTGCCTCTATTCCAGCTCCTTACCACAGCGGTG GTGCTTAAAGAAAGGATCATCAGCAACAGGTCAGGATAGTTCTACCTTTGGGATAGGG CTGCTTTCCCCGTGCTAGTATTTCTGTGACTGTTAGTGGCACTGAGGACTGCAAACTTT TATGCAATATTCTTAATACCCTATTGATATTATGCACTTTAATCATTCCAAAGAAGCCAAG AATGCTGTATAGTGATGATTCCTTCCTAATGAATTCATCTTAACTATTTAGAATGTTATGT CCCTTTTCTTTTGGATAGCCAACTTGGTATAAATGTTATATGGATTTTTCTAAAATGACTA TATAGGACTTAAGACTTTGAAATGTAATTTACTTATAAGGGGAAATAATTATGCTTTAGC ACATCATTTTAGAAACGTCACATTTTAGAAACATTCAGCTTGCTAACCTACATGTTTGGG AATTCATTAAAACCAGTTGTCTATATATTTTGTGCCATGTATATAAGAACATTACAATATA TCTTTTTCTACATATGTAGTATGTGCAACCAGTGGTTCTCAGAGTATGGTTCTCAGCCCA CCAGCTAGTATCAGTATCACCTGGGAACTAGTTAGAAATGTAAATTCTTTGGCCCCATC CCAGACATACTGAGTCAGAAACTCTGGAATAGGGCCCCCGCAATCTGTTTTCACAAGC CCTCCAGGTGATTCTGATGCACACTTTAAAGTTTAGGAACCACTGGGCTAAGACTCTGT TGAGATATAGAGTTTTTCTTCCACTCAGACTGATATAGTTATACATTGTTCTTCATGTAAA TTCAGCTTAACCTGGTTATCTATAATCTTTTATTGGCAAAAGTTAATTCTCAGTACTGCCT ATAGAGATACAGTGTATTTTATGTACATACACAATTAGTCTAATTCTTGATAATTCAGTTA ATTTAGTTTGGCATTTTCCTACCACTTACTAAAAGGTTTACATTAAATGACTGATTTAAAT ATATAGGTGCAATGTTCTATGTTTATTTTAATTGTTATGACATTTAAGTAGCTAATATAAT TGACCGGTGCTAAAGTCTCCTGTTTATCCATAAAATGGGTACATTATGGGCAGTGTAAT ACAAGCTTTCTTTTCATTGCCTAGTACTTTACCAGCAGACCACAGTTTTGCCCTGGCTA GACCAACCCTCAGAACAAAATCATCATTCCTTGTATTTATATTTGTATCTGAGATAGTAA ACAAGATGGCTGGCCAGGTCAACATGGCACCTTAACTTATTTTTTTAATAGGTAAAACTT CTTCAAAAGTAGCTTGCTTTGTATAAGAACTAAGCTATCAGTATAGATATAGCTATCCTT GGAGCTTATGTTTCAGACAAGAATTATTTACTAAAATAAATAATAAACAAGATAATGCATT ATACAATTTGGGCATTTCTCGTTTCTCAAGTGTATGCATCATGGTAAATATAAACTAACC ACAAGATAGGTAGATTGATTCATTTCATTTTAATCTCCTTGTGTAATTCAGTACCTCCATA ATTGTTCTAATCTTCTTCCCACTGTTTACAAATTACCAGTTAATTAACTCGTGAAAGAAAA ATTCACATATCAGAATAAAAATAAATGTATACTCACTTTATAAAAATCACCACTGCTGTCT TTCCTTAATACTAGCAGTGGAAATGTAAGTGGCTTACTCTACAAATTTTGGTGCTGGCAA ATACATAGGCAAACTGTTGGGAGCTGCTCTAGTTACATTCCTCCCTTCTTATTCCCTTTT TCTCTTCCTCACTTTATTGCATAACATATTCCTGTACCCAAAGCATTCTACCACAGTTCT ATTTGACTCCCACTTGTAATAACTCCTTTAAAAAATTCCATGTTTAACCATATGACCCTG CTTGCTTACTCATATTCTCCCTCCCTCTCCCCTTCCTTTCTCTCTCTTCCAGAAGTCATT TGCCTGGTTTGAAATATTTTGTAGGGATTGCTTATTATATTATTTTAGCTGATGAACCTCA GGACAACGTCTACACACACACACATACATACACGCACACAAAATCTCAGCTGTTGAAGA GTGGGCTTGGAATCAGACTTCTGTGTCCAGTAAAAAACTCCTGCACTGAAGTCATTGTG ACTTGAGTAGTTACAGACTGATTCCAGTGAACTTGATCTAATTTCTTTTGATCTAATGAA TGTGTCTGCTTACCTTGTTTCCTTTTAATTGATAAGCTCCAAGTAGTTGCTAATTTTTTGA CAACTTTAAATGAGTTTCATTCACTTCTTTTACTTAATGTTTTAAGTATAGTACCAATAATT TCATTAACCTGTTCTCAAGTGGTTTAGCTACCATTCTGCCATTTTTAATTTTTATTTAATTT TATTTGCTTGAGCACACTGATCAACCACTGAACTGCCTTCTTCCATTGTCCTGCAATGAT ATAAGGGTTACATTTTTGTGTATATGGCTTTCATAGTTGGGATTTCAGAGCACTGATACC AGATATTTTCAGTTTGTTCTCTGGGGGAATTTCATTTGCATCTATGTTTTTAGCTATCTGT GATAACTTGTTAAATATTAAAAAGATATTTTGCTTCTATTGGAACATTTGTATACTCGCAA CTATATTTCTGTAAACAGCTGCAGTCAAAAATAAAACACTGAAAGTTTTCA. (SEQ ID NO:286; NM_001113178.3), or a nucleic acid sequence that hybridizes to a nucleic acid sequence consisting of SEQ ID NO:286 under stringent hybridization conditions

In some embodiments, BCL6 transcription repressor (BCL6, BCL5, LAZ3, BCL6A, ZNF51, ZBTB27) comprises the amino acid sequence: MASPADSCIQFTRHASDVLLNLNRLRSRDILTDVVIVVSREQFRAHKTVLMACSGLFYSIFTD QLKCNLSVINLDPEINPEGFCILLDFMYTSRLNLREGNIMAVMATAMYLQMEHVVDTCRKFI KASEAEMVSAIKPPREEFLNSRMLMPQDIMAYRGREVVENNLPLRSAPGCESRAFAPSLYS GLSTPPASYSMYSHLPVSSLLFSDEEFRDVRMPVANPFPKERALPCDSARPVPGEYSRPTL EVSPNVCHSNIYSPKETIPEEARSDMHYSVAEGLKPAAPSARNAPYFPCDKASKEEERPSS EDEIALHFEPPNAPLNRKGLVSPQSPQKSDCQPNSPTESCSSKNACILQASGSPPAKSPTD PKACNWKKYKFIVLNSLNQNAKPEGPEQAELGRLSPRAYTAPPACQPPMEPENLDLQSPT KLSASGEDSTIPQASRLNNIVNRSMTGSPRSSSESHSPLYMHPPKCTSCGSQSPQHAEMC LHTAGPTFPEEMGETQSEYSDSSCENGAFFCNECDCRFSEEASLKRHTLQTHSDKPYKCD RCQASFRYKGNLASHKTVHTGEKPYRCNICGAQFNRPANLKTHTRIHSGEKPYKCETCGA RFVQVAHLRAHVLIHTGEKPYPCEICGTRFRHLQTLKSHLRIHTGEKPYHCEKCNLHFRHKS QLRLHLRQKHGAITNTKVQYRVSATDLPPELPKAC. (SEQ ID NO:287; NP_001124317.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:287)

In some embodiments, the nucleic acid sequence encoding BCL6 comprises the nucleic acid sequence: ACAAGCGAGCTGGTGGTTGAAGCTGGTTAAAGAACAGCCTAGGTATTCCAGAAGTGTT TGAGGATCCCTTCCATGAAGGAAGAGAGGAAAGTTTTTAAGTAAACCTCCCACTCCCAT GTGTCTTCAGCTTTCTTTTGCAAAGGAGAAAATCCTTGAAGTTTGGTAAAGACCGAGTT AGTCTATCTCTCTTTGCCTATCTCGAGTTGGGCTGGGGAGAGGAGGAGATAGGTTCTTT TGTCTTTTTCTGTCTTCTCCCTTCCCCACTTCCTTCCCTCCAGTCCCCACTCACTCACAT GCACACACTAACCTTGGAGCCGATGGGATTGAGTGACTGGCACTTGGGACCACAGAGA AATGTCAGAGTGTTTGGTTACAGACTCAAGGAAACCTCTCATTTTAGAGTGCTCATTTG GTTTTGAGCAAAATTTTGGACTGTGAAGCAAGGCATTGGTGAAGACAAAATGGCCTCGC CGGCTGACAGCTGTATCCAGTTCACCCGCCATGCCAGTGATGTTCTTCTCAACCTTAAT CGTCTCCGGAGTCGAGACATCTTGACTGATGTTGTCATTGTTGTGAGCCGTGAGCAGT TTAGAGCCCATAAAACGGTCCTCATGGCCTGCAGTGGCCTGTTCTATAGCATCTTTACA GACCAGTTGAAATGCAACCTTAGTGTGATCAATCTAGATCCTGAGATCAACCCTGAGGG ATTCTGCATCCTCCTGGACTTCATGTACACATCTCGGCTCAATTTGCGGGAGGGCAACA TCATGGCTGTGATGGCCACGGCTATGTACCTGCAGATGGAGCATGTTGTGGACACTTG CCGGAAGTTTATTAAGGCCAGTGAAGCAGAGATGGTTTCTGCCATCAAGCCTCCTCGT GAAGAGTTCCTCAACAGCCGGATGCTGATGCCCCAAGACATCATGGCCTATCGGGGTC GTGAGGTGGTGGAGAACAACCTGCCACTGAGGAGCGCCCCTGGGTGTGAGAGCAGAG CCTTTGCCCCCAGCCTGTACAGTGGCCTGTCCACACCGCCAGCCTCTTATTCCATGTA CAGCCACCTCCCTGTCAGCAGCCTCCTCTTCTCCGATGAGGAGTTTCGGGATGTCCGG ATGCCTGTGGCCAACCCCTTCCCCAAGGAGCGGGCACTCCCATGTGATAGTGCCAGG CCAGTCCCTGGTGAGTACAGCCGGCCGACTTTGGAGGTGTCCCCCAATGTGTGCCAC AGCAATATCTATTCACCCAAGGAAACAATCCCAGAAGAGGCACGAAGTGATATGCACTA CAGTGTGGCTGAGGGCCTCAAACCTGCTGCCCCCTCAGCCCGAAATGCCCCCTACTTC CCTTGTGACAAGGCCAGCAAAGAAGAAGAGAGACCCTCCTCGGAAGATGAGATTGCCC TGCATTTCGAGCCCCCCAATGCACCCCTGAACCGGAAGGGTCTGGTTAGTCCACAGAG CCCCCAGAAATCTGACTGCCAGCCCAACTCGCCCACAGAGTCCTGCAGCAGTAAGAAT GCCTGCATCCTCCAGGCTTCTGGCTCCCCTCCAGCCAAGAGCCCCACTGACCCCAAAG CCTGCAACTGGAAGAAATACAAGTTCATCGTGCTCAACAGCCTCAACCAGAATGCCAAA CCAGAGGGGCCTGAGCAGGCTGAGCTGGGCCGCCTTTCCCCACGAGCCTACACGGC CCCACCTGCCTGCCAGCCACCCATGGAGCCTGAGAACCTTGACCTCCAGTCCCCAACC AAGCTGAGTGCCAGCGGGGAGGACTCCACCATCCCACAAGCCAGCCGGCTCAATAAC ATCGTTAACAGGTCCATGACGGGCTCTCCCCGCAGCAGCAGCGAGAGCCACTCACCA CTCTACATGCACCCCCCGAAGTGCACGTCCTGCGGCTCTCAGTCCCCACAGCATGCAG AGATGTGCCTCCACACCGCTGGCCCCACGTTCCCTGAGGAGATGGGAGAGACCCAGT CTGAGTACTCAGATTCTAGCTGTGAGAACGGGGCCTTCTTCTGCAATGAGTGTGACTG CCGCTTCTCTGAGGAGGCCTCACTCAAGAGGCACACGCTGCAGACCCACAGTGACAAA CCCTACAAGTGTGACCGCTGCCAGGCCTCCTTCCGCTACAAGGGCAACCTCGCCAGC CACAAGACCGTCCATACCGGTGAGAAACCCTATCGTTGCAACATCTGTGGGGCCCAGT TCAACCGGCCAGCCAACCTGAAAACCCACACTCGAATTCACTCTGGAGAGAAGCCCTA CAAATGCGAAACCTGCGGAGCCAGATTTGTACAGGTGGCCCACCTCCGTGCCCATGTG CTTATCCACACTGGTGAGAAGCCCTATCCCTGTGAAATCTGTGGCACCCGTTTCCGGC ACCTTCAGACTCTGAAGAGCCACCTGCGAATCCACACAGGAGAGAAACCTTACCATTG TGAGAAGTGTAACCTGCATTTCCGTCACAAAAGCCAGCTGCGACTTCACTTGCGCCAG AAGCATGGCGCCATCACCAACACCAAGGTGCAATACCGCGTGTCAGCCACTGACCTGC CTCCGGAGCTCCCCAAAGCCTGCTGAAGCATGGAGTGTTGATGCTTTCGTCTCCAGCC CCTTCTCAGAATCTACCCAAAGGATACTGTAACACTTTACAATGTTCATCCCATGATGTA GTGCCTCTTTCATCCACTAGTGCAAATCATAGCTGGGGGTTGGGGGTGGTGGGGGTC GGGGCCTGGGGGACTGGGAGCCGCAGCAGCTCCCCCTCCCCCACTGCCATAAAACAT TAAGAAAATCATATTGCTTCTTCTCCTATGTGTAAGGTGAACCATGTCAGCAAAAAGCAA AATCATTTTATATGTCAAAGCAGGGGAGTATGCAAAAGTTCTGACTTGACTTTAGTCTGC AAAATGAGGAATGTATATGTTTTGTGGGAACAGATGTTTCTTTTGTATGTAAATGTGCAT TCTTTTAAAAGACAAGACTTCAGTATGTTGTCAAAGAGAGGGCTTTAATTTTTTTAACCA AAGGTGAAGGAATATATGGCAGAGTTGTAAATATATAAATATATATATATATAAAATAAAT ATATATAAACCTAAAAAAGATATATTAAAAATATAAAACTGCGTTAAAGGCTCGATTTTGT ATCTGCAGGCAGACACGGATCTGAGAATCTTTATTGAGAAAGAGCACTTAAGAGAATAT TTTAAGTATTGCATCTGTATAAGTAAGAAAATATTTTGTCTAAAATGCCTCAGTGTATTTG TATTTTTTTGCAAGTGAAGGTTTACAATTTACAAAGTGTGTATTAAAAAAAACAAAAAGAA CAAAAAAATCTGCAGAAGGAAAAATGTGTAATTTTGTTCTAGTTTTCAGTTTGTATATAC CCGTACAACGTGTCCTCACGGTGCCTTTTTTCACGGAAGTTTTCAATGATGGGCGAGC GTGCACCATCCCTTTTTGAAGTGTAGGCAGACACAGGGACTTGAAGTTGTTACTAACTA AACTCTCTTTGGGAATGTTTGTCTCATCCCATTCTGCGTCATGCTTGTGTTATAACTACT CCGGAGACAGGGTTTGGCTGTGTCTAAACTGCATTACCGCGTTGTAAAATATAGCTGTA CAAATATAAGAATAAAATGTTGAAAAGTCAAA. (SEQ ID NO:288; NM_001130845.2), or a nucleic acid sequence that hybridizes to a nucleic acid sequence consisting of SEQ ID NO:288 under stringent hybridization conditions

In some embodiments, myogenic differentiation 1 (MYOD1, PUM, MYF3, MYOD, bHLHc1) comprises the amino acid sequence: MELLSPPLRDVDLTAPDGSLCSFATTDDFYDDPCFDSPDLRFFEDLDPRLMHVGALLKPEE HSHFPAAVHPAPGAREDEHVRAPSGHHQAGRCLLWACKACKRKTTNADRRKAATMRER RRLSKVNEAFETLKRCTSSNPNQRLPKVEILRNAIRYIEGLQALLRDQDAAPPGAAAAFYAP GPLPPGRGGEHYSGDSDASSPRSNCSDGMMDYSGPPSGARRRNCYEGAYYNEAPSEPR PGKSAAVSSLDCLSSIVERISTESPAAPALLLADVPSESPPRRQEAAAPSEGESSGDPTQSP DAAPQCPAGANPNPIYQVL. (SEQ ID NO:289; NP_002469.2), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:289)

In some embodiments, the nucleic acid sequence encoding MYOD comprises the nucleic acid sequence: AGGGGTGAGGAAGCCCTGGGGCGCTGCCGCCGCTTTCCTTAACCACAAATCAGGCCG GACAGGAGAGGGAGGGGTGGGGGACAGTGGGTGGGCATTCAGACTGCCAGCACTTT GCTATCTACAGCCGGGGCTCCCGAGCGGCAGAAAGTTCCGGCCACTCTCTGCCGCTT GGGTTGGGCGAAGCCAGGACCGTGCCGCGCCACCGCCAGGATATGGAGCTACTGTC GCCACCGCTCCGCGACGTAGACCTGACGGCCCCCGACGGCTCTCTCTGCTCCTTTGC CACAACGGACGACTTCTATGACGACCCGTGTTTCGACTCCCCGGACCTGCGCTTCTTC GAAGACCTGGACCCGCGCCTGATGCACGTGGGCGCGCTCCTGAAACCCGAAGAGCAC TCGCACTTCCCCGCGGCGGTGCACCCGGCCCCGGGCGCACGTGAGGACGAGCATGT GCGCGCGCCCAGCGGGCACCACCAGGCGGGCCGCTGCCTACTGTGGGCCTGCAAGG CGTGCAAGCGCAAGACCACCAACGCCGACCGCCGCAAGGCCGCCACCATGCGCGAG CGGCGCCGCCTGAGCAAAGTAAATGAGGCCTTTGAGACACTCAAGCGCTGCACGTCG AGCAATCCAAACCAGCGGTTGCCCAAGGTGGAGATCCTGCGCAACGCCATCCGCTATA TCGAGGGCCTGCAGGCTCTGCTGCGCGACCAGGACGCCGCGCCCCCTGGCGCCGCA GCCGCCTTCTATGCGCCGGGCCCGCTGCCCCCGGGCCGCGGCGGCGAGCACTACAG CGGCGACTCCGACGCGTCCAGCCCGCGCTCCAACTGCTCCGACGGCATGATGGACTA CAGCGGCCCCCCGAGCGGCGCCCGGCGGCGGAACTGCTACGAAGGCGCCTACTACA ACGAGGCGCCCAGCGAACCCAGGCCCGGGAAGAGTGCGGCGGTGTCGAGCCTAGAC TGCCTGTCCAGCATCGTGGAGCGCATCTCCACCGAGAGCCCTGCGGCGCCCGCCCTC CTGCTGGCGGACGTGCCTTCTGAGTCGCCTCCGCGCAGGCAAGAGGCTGCCGCCCCC AGCGAGGGAGAGAGCAGCGGCGACCCCACCCAGTCACCGGACGCCGCCCCGCAGTG CCCTGCGGGTGCGAACCCCAACCCGATATACCAGGTGCTCTGAGGGGATGGTGGCCG CCCACCCGCCCGAGGGATGGTGCCCCTAGGGTCCCTCGCGCCCAAAAGATTGAACTT AAATGCCCCCCTCCCAACAGCGCTTTAAAAGCGACCTCTCTTGAGGTAGGAGAGGCGG GAGAACTGAAGTTTCCGCCCCCGCCCCACAGGGCAAGGACACAGCGCGGTTTTTTCCA CGCAGCACCCTTCTCGGAGACCCATTGCGATGGCCGCTCCGTGTTCCTCGGTGGGCC AGAGCTGAACCTTGAGGGGCTAGGTTCAGCTTTCTCGCGCCCTCCCCCATGGGGGTG AGACCCTCGCAGACCTAAGCCCTGCCCCGGGATGCACCGGTTATTTGGGGGGGCGTG AGACCCAGTGCACTCCGGTCCCAAATGTAGCAGGTGTAACCGTAACCCACCCCCAACC CGTTTCCCGGTTCAGGACCACTTTTTGTAATACTTTTGTAATCTATTCCTGTAAATAAGA GTTGCTTTGCCAGAGCAGGAGCCCCTGGGGCTGTATTTATCTCTGAGGCATGGTGTGT GGTGCTACAGGGAATTTGTACGTTTATACCGCAGGCGGGCGAGCCGCGGGCGCTCGC TCAGGTGATCAAAATAAAGGCGCTAATTTATACCGCC. (SEQ ID NO:290; NM_002478.5), or a nucleic acid sequence that hybridizes to a nucleic acid sequence consisting of SEQ ID NO:290 under stringent hybridization conditions

In some embodiments, myogenic factor 5 (MYF5, EORVA, bHLHc2) comprises the amino acid sequence: MDVMDGCQFSPSEYFYDGSCIPSPEGEFGDEFVPRVAAFGAHKAELQGSDEDEHVRAPT GHHQAGHCLMWACKACKRKSTTMDRRKAATMRERRRLKKVNQAFETLKRCTTTNPNQRL PKVEILRNAIRYIESLQELLREQVENYYSLPGQSCSEPTSPTSNCSDGMPECNSPVWSRKS STFDSIYCPDVSNVYATDKNSLSSLDCLSNIVDRITSSEQPGLPLQDLASLSPVASTDSQPAT PGASSSRLIYHVL. (SEQ ID NO:291; NP_005584.2), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:291)

In some embodiments, the nucleic acid sequence encoding MYF5 comprises the nucleic acid sequence: ACCCAGGCCAACAGGCGTCTGCCCTTGTTAATTACCGGAGCGACAGACTAGGGAGCTC CGCCCGGGATTTGCCCATCGGCGGAGGCGCCAGGCTCCCGTTTCTCCCCATCCCTCT CGCTGCCGTCCAGGTGCACCGCCTGCCTCTCAGCAGGATGGACGTGATGGATGGCTG CCAGTTCTCACCTTCTGAGTACTTCTACGACGGCTCCTGCATACCGTCCCCCGAGGGT GAATTTGGGGACGAGTTTGTGCCGCGAGTGGCTGCCTTCGGAGCGCACAAAGCAGAG CTGCAGGGCTCAGATGAGGACGAGCACGTGCGAGCGCCTACCGGCCACCACCAGGCT GGTCACTGCCTCATGTGGGCCTGCAAAGCCTGCAAGAGGAAGTCCACCACCATGGATC GGCGGAAGGCAGCCACTATGCGCGAGCGGAGGCGCCTGAAGAAGGTCAACCAGGCT TTCGAAACCCTCAAGAGGTGTACCACGACCAACCCCAACCAGAGGCTGCCCAAGGTG GAGATCCTCAGGAATGCCATCCGCTACATCGAGAGCCTGCAGGAGTTGCTGAGAGAG CAGGTGGAGAACTACTATAGCCTGCCGGGACAGAGCTGCTCGGAGCCCACCAGCCCC ACCTCCAACTGCTCTGATGGCATGCCCGAATGTAACAGTCCTGTCTGGTCCAGAAAGA GCAGTACTTTTGACAGCATCTACTGTCCTGATGTATCAAATGTATATGCCACAGATAAAA ACTCCTTATCCAGCTTGGATTGCTTATCCAACATAGTGGACCGGATCACCTCCTCAGAG CAACCTGGGTTGCCTCTCCAGGATCTGGCTTCTCTCTCTCCAGTTGCCAGCACCGATT CACAGCCTGCAACTCCAGGGGCTTCTAGTTCCAGGCTTATCTATCATGTGCTATGAACT AATTTTCTGGTCTATATGACTTCTTCCAGGAGGGCCTAATACACAGGAAGAAGAAGGCT TCAAAAAGTCCCAAACCAAGACAACATGTACATAAAGATTTCTTTTCAGTTGTAAATTTG TAAAGATTACCTTGCCACTTTATAAGAAAGTGTATTTAACTAAAAAGTCATCATTGCAAAT AATACTTTCTTCTTCTTTATTATTCTTTGCTTAGATATTAATACATAGTTCCAGTAATACTA TTTCTGATAGGGGGCCATTGATTGAGGGTAGCTTGTTGCAATGCTTAACTTATATATACA TATATATATATTATAAATATTGCTCATCAAAATGTCTCTGGTGTTTAGAGCTTTATTTTTTT CTTTAAAACATTAAAACAGCTGAGAATCAGTTAAATGGAATTTTAAATATATTTAACTATT TCTTTTCTCTTTAATCCTTTAGTTATATTGTATTAAATAAAAATATAATACTGCCTAATGTA TATATTTTGATCTTTTCTTGTAAGAAATGTATCTTTTAAATGTAAGCACAAAATAGTACTTT GTGGATCATTTCAAGATATAAGAAATTTTGGAAATTCCACCATAAATAAAATTTTTTACTA CAAGAAA. (SEQ ID NO:292; NM_005593.3), or a nucleic acid sequence that hybridizes to a nucleic acid sequence consisting of SEQ ID NO:292 under stringent hybridization conditions

In some embodiments, myogenin (MYOG, MYF5, bHLHc3) comprises the amino acid sequence: MELYETSPYFYQEPRFYDGENYLPVHLQGFEPPGYERTELTLSPEAPGPLEDKGLGTPEH CPGQCLPWACKVCKRKSVSVDRRRAATLREKRRLKKVNEAFEALKRSTLLNPNQRLPKVEI LRSAIQYIERLQALLSSLNQEERDLRYRGGGGPQPGVPSECSSHSASCSPEWGSALEFSA NPGDHLLTADPTDAHNLHSLTSIVDSITVEDVSVAFPDETMPN. (SEQ ID NO:293; NP_002470.2), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:293)

In some embodiments, the nucleic acid sequence encoding MYOG comprises the nucleic acid sequence: GGGCTGCTGGAGCTTGGGGGCTGGTGGCAGGAACAAGCCTTTTCCGACCCCATGGAG CTGTATGAGACATCCCCCTACTTCTACCAGGAACCCCGCTTCTATGATGGGGAAAACTA CCTGCCTGTCCACCTCCAGGGCTTCGAACCACCAGGCTACGAGCGGACGGAGCTCAC CCTGAGCCCCGAGGCCCCAGGGCCCCTTGAGGACAAGGGGCTGGGGACCCCCGAGC ACTGTCCAGGCCAGTGCCTGCCGTGGGCGTGTAAGGTGTGTAAGAGGAAGTCGGTGT CCGTGGACCGGCGGCGGGCGGCCACACTGAGGGAGAAGCGCAGGCTCAAGAAGGTG AATGAGGCCTTCGAGGCCCTGAAGAGAAGCACCCTGCTCAACCCCAACCAGCGGCTG CCCAAGGTGGAGATCCTGCGCAGTGCCATCCAGTACATCGAGCGCCTCCAGGCCCTG CTCAGCTCCCTCAACCAGGAGGAGCGTGACCTCCGCTACCGGGGCGGGGGCGGGCC CCAGCCAGGGGTGCCCAGCGAATGCAGCTCTCACAGCGCCTCCTGCAGTCCAGAGTG GGGCAGTGCACTGGAGTTCAGCGCCAACCCAGGGGATCATCTGCTCACGGCTGACCC TACAGATGCCCACAACCTGCACTCCCTCACCTCCATCGTGGACAGCATCACAGTGGAA GATGTGTCTGTGGCCTTCCCAGATGAAACCATGCCCAACTGAGATTGTCTTCCAAGCC GGGCATCCTTGCGAGCCCCCCAAGCTGGCCACAGATGCCACTACTTCTGTAGCAGGG GCCTCCTAAGCCAGGCTGCCCTGATGCTAGGAAGCCAGCTCTGGGGTGCCATAGGCC AGACTATCCCCTTCCTCATCCATGTAAGGTTAACCCACCCCCCAGCAAGGGACTGGAC GCCCTCATTCAGCTGCCTCCTTAGAGGAGAGGGCATCCCCTTTCCAGGGAGGTAAAGC AGGGGACCAGAGCGCCCCCTCGTGTATGCCCCAGCTCAGGGGGCAAACTCAGGAGCT TCCTTTTTATCATAACGCGGCCTCTAATTCCACCCCCCAAGTGAAACGGTTTGAGAGAC GCAGTGCCCTGACCTGGACAAGCTGTGCACGTCTCCTGTTCTGGTCTCTTCCCGATGC CAGTGGCTGGGCTGGGCCTGCCCTGAATTGAGAGAGAAGAAGGGGAGAGGAACAGCC CTCTGTTCCCAAGTCCCTGGGGGGCCAAACTTTTGCAGTGAATATTGGGAACCTTCCA GTGGTTTTATGTTTTGTTTTGTTTCGTGTGTTGTTTGTAAAGCTGCCATCCGACCAAGGT CTCCTGTGCTGAAGTTGCCGGGGACAGGCAGGGAAAAGGGGTTGGGGCCTCTTGGGG GTGATTTCTTTTGTTAACAAAGCATTGTGTGGTTTTGCCATTGTTTTGTATTTTTTTTTTTT TTTTTTTTTTTTGCTAACTTATTTGGATTTCCTTTTTTAAAAAATGAATAAAGACTGGTTGC CAGAA. (SEQ ID NO:294; NM_002479.6), or a nucleic acid sequence that hybridizes to a nucleic acid sequence consisting of SEQ ID NO:294 under stringent hybridization conditions

In some embodiments, Spi-1 proto-oncogene (SP11, OF, PU.1, SFP11, SPI-A) comprises the amino acid sequence: MLQACKMEGFPLVPPQPSEDLVPYDTDLYQRQTHEYYPYLSSDGESHSDHYWDFHPHHV HSEFESFAENNFTELQSVQPPQLQQLYRHMELEQMHVLDTPMVPPHPSLGHQVSYLPRM CLQYPSLSPAQPSSDEEEGERQSPPLEVSDGEADGLEPGPGLLPGETGSKKKIRLYQFLLD LLRSGDMKDSIWWVDKDKGTFQFSSKHKEALAHRWGIQKGNRKKMTYQKMARALRNYGK TGEVKKVKKKLTYQFSGEVLGRGGLAERRHPPH. (SEQ ID NO:295; NP_001074016.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:295)

In some embodiments, the nucleic acid sequence encoding PU.1 comprises the nucleic acid sequence: AAAATCAGGAACTTGTGCTGGCCCTGCAATGTCAAGGGAGGGGGCTCACCCAGGGCT CCTGTAGCTCAGGGGGCAGGCCTGAGCCCTGCACCCGCCCCACGACCGTCCAGCCC CTGACGGGGCACCCCATCCTGAGGGGCTCTGCATTGGCCCCCACCGAGGCAGGGGAT CTGACCGACTCGGAGCCCGGCTGGATGTTACAGGCGTGCAAAATGGAAGGGTTTCCC CTCGTCCCCCCTCAGCCATCAGAAGACCTGGTGCCCTATGACACGGATCTATACCAAC GCCAAACGCACGAGTATTACCCCTATCTCAGCAGTGATGGGGAGAGCCATAGCGACCA TTACTGGGACTTCCACCCCCACCACGTGCACAGCGAGTTCGAGAGCTTCGCCGAGAAC AACTTCACGGAGCTCCAGAGCGTGCAGCCCCCGCAGCTGCAGCAGCTCTACCGCCAC ATGGAGCTGGAGCAGATGCACGTCCTCGATACCCCCATGGTGCCACCCCATCCCAGTC TTGGCCACCAGGTCTCCTACCTGCCCCGGATGTGCCTCCAGTACCCATCCCTGTCCCC AGCCCAGCCCAGCTCAGATGAGGAGGAGGGCGAGCGGCAGAGCCCCCCACTGGAGG TGTCTGACGGCGAGGCGGATGGCCTGGAGCCCGGGCCTGGGCTCCTGCCTGGGGAG ACAGGCAGCAAGAAGAAGATCCGCCTGTACCAGTTCCTGTTGGACCTGCTCCGCAGCG GCGACATGAAGGACAGCATCTGGTGGGTGGACAAGGACAAGGGCACCTTCCAGTTCT CGTCCAAGCACAAGGAGGCGCTGGCGCACCGCTGGGGCATCCAGAAGGGCAACCGC AAGAAGATGACCTACCAGAAGATGGCGCGCGCGCTGCGCAACTACGGCAAGACGGGC GAGGTCAAGAAGGTGAAGAAGAAGCTCACCTACCAGTTCAGCGGCGAAGTGCTGGGC CGCGGGGGCCTGGCCGAGCGGCGCCACCCGCCCCACTGAGCCCGCAGCCCCCGCC GGGCCCCGCCAGGCCTCCCCGCTGGCCATAGCATTAAGCCCTCGCCCGGCCCGGAC ACAGGGAGGACGCTCCCGGGGCCCAGAGGCAGGACTGTGGCGGGCCGGGCCTCGC CTCACCCGCCCCCTCCCCCCACTCCAGGCCCCCTCCACATCCCGCTTCGCCTCCCTCC AGGACTCCACCCCGGCTCCCGGACGCCAGCTGGGCGTCAGACCCCACCGGGGCAAC CTTGCAGAGGACGACCCGGGGTACTGCCTTGGGAGTCTCAAGTCCGTATGTAAATCAG ATCTCCCCTCTCACCCCTCCCACCCATTAACCTCCTCCCAAAAAACAAGTAAAGTTATTC TCAATCCA. (SEQ ID NO:296; NM_001080547.2), or a nucleic acid sequence that hybridizes to a nucleic acid sequence consisting of SEQ ID NO:296 under stringent hybridization conditions

In some embodiments, colony stimulating factor 1 receptor (CSF1R, FMS, CSFR, FIM2, HDLS, CD115, CSF-1R, BANDDOS, M-CSF-R) comprises the amino acid sequence: MGPGVLLLLLVATAWHGQGIPVIEPSVPELVVKPGATVTLRCVGNGSVEWDGPPSPHWTL YSDGSSSILSTNNATFQNTGTYRCTEPGDPLGGSAAIHLYVKDPARPWNVLAQEVVVFEDQ DALLPCLLTDPVLEAGVSLVRVRGRPLMRHTNYSFSPWHGFTIHRAKFIQSQDYQCSALMG GRKVMSISIRLKVQKVIPGPPALTLVPAELVRIRGEAAQIVCSASSVDVNFDVFLQHNNTKLAI PQQSDFHNNRYQKVLTLNLDQVDFQHAGNYSCVASNVQGKHSTSMFFRVVESAYLNLSS EQNLIQEVTVGEGLNLKVMVEAYPGLQGFNWTYLGPFSDHQPEPKLANATTKDTYRHTFTL SLPRLKPSEAGRYSFLARNPGGWRALTFELTLRYPPEVSVIWTFINGSGTLLCAASGYPQP NVTWLQCSGHTDRCDEAQVLQVWDDPYPEVLSQEPFHKVTVQSLLTVETLEHNQTYECR AHNSVGSGSWAFIPISAGAHTHPPDEFLFTPVVVACMSIMALLLLLLLLLLYKYKQKPKYQVR WKIIESYEGNSYTFIDPTQLPYNEKWEFPRNNLQFGKTLGAGAFGKVVEATAFGLGKEDAV LKVAVKMLKSTAHADEKEALMSELKIMSHLGQHENIVNLLGACTHGGPVLVITEYCCYGDLL NFLRRKAEAMLGPSLSPGQDPEGGVDYKNIHLEKKYVRRDSGFSSQGVDTYVEMRPVSTS SNDSFSEQDLDKEDGRPLELRDLLHFSSQVAQGMAFLASKNCIHRDVAARNVLLTNGHVA KIGDFGLARDIMNDSNYIVKGNARLPVKWMAPESIFDCVYTVQSDVWSYGILLWEIFSLGLN PYPGILVNSKFYKLVKDGYQMAQPAFAPKNIYSIMQACWALEPTHRPTFQQICSFLQEQAQ EDRRERDYTNLPSSSRSGGSGSSSSELEEESSSEHLTCCEQGDIAQPLLQPNNYQFC. (SEQ ID NO:297; NP_001275634.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:297)

In some embodiments, the nucleic acid sequence encoding CSFR comprises the nucleic acid sequence: AAACAGCCAGTGCAGAGGAGAGGAACGTGTGTCCAGTGTCCCGATCCCTGCGGAGCT AGTAGCTGAGAGCTCTGTGCCCTGGGCACCTTGCAGCCCTGCACCTGCCTGCCACTTC CCCACCGAGGCCATGGGCCCAGGAGTTCTGCTGCTCCTGCTGGTGGCCACAGCTTGG CATGGTCAGGGAATCCCAGTGATAGAGCCCAGTGTCCCTGAGCTGGTCGTGAAGCCA GGAGCAACGGTGACCTTGCGATGTGTGGGCAATGGCAGCGTGGAATGGGATGGCCCC CCATCACCTCACTGGACCCTGTACTCTGATGGCTCCAGCAGCATCCTCAGCACCAACA ACGCTACCTTCCAAAACACGGGGACCTATCGCTGCACTGAGCCTGGAGACCCCCTGG GAGGCAGCGCCGCCATCCACCTCTATGTCAAAGACCCTGCCCGGCCCTGGAACGTGC TAGCACAGGAGGTGGTCGTGTTCGAGGACCAGGACGCACTACTGCCCTGTCTGCTCA CAGACCCGGTGCTGGAAGCAGGCGTCTCGCTGGTGCGTGTGCGTGGCCGGCCCCTC ATGCGCCACACCAACTACTCCTTCTCGCCCTGGCATGGCTTCACCATCCACAGGGCCA AGTTCATTCAGAGCCAGGACTATCAATGCAGTGCCCTGATGGGTGGCAGGAAGGTGAT GTCCATCAGCATCCGGCTGAAAGTGCAGAAAGTCATCCCAGGGCCCCCAGCCTTGACA CTGGTGCCTGCAGAGCTGGTGCGGATTCGAGGGGAGGCTGCCCAGATCGTGTGCTCA GCCAGCAGCGTTGATGTTAACTTTGATGTCTTCCTCCAACACAACAACACCAAGCTCGC AATCCCTCAACAATCTGACTTTCATAATAACCGTTACCAAAAAGTCCTGACCCTCAACCT CGATCAAGTAGATTTCCAACATGCCGGCAACTACTCCTGCGTGGCCAGCAACGTGCAG GGCAAGCACTCCACCTCCATGTTCTTCCGGGTGGTAGAGAGTGCCTACTTGAACTTGA GCTCTGAGCAGAACCTCATCCAGGAGGTGACCGTGGGGGAGGGGCTCAACCTCAAAG TCATGGTGGAGGCCTACCCAGGCCTGCAAGGTTTTAACTGGACCTACCTGGGACCCTT TTCTGACCACCAGCCTGAGCCCAAGCTTGCTAATGCTACCACCAAGGACACATACAGG CACACCTTCACCCTCTCTCTGCCCCGCCTGAAGCCCTCTGAGGCTGGCCGCTACTCCT TCCTGGCCAGAAACCCAGGAGGCTGGAGAGCTCTGACGTTTGAGCTCACCCTTCGATA CCCCCCAGAGGTAAGCGTCATATGGACATTCATCAACGGCTCTGGCACCCTTTTGTGT GCTGCCTCTGGGTACCCCCAGCCCAACGTGACATGGCTGCAGTGCAGTGGCCACACT GATAGGTGTGATGAGGCCCAAGTGCTGCAGGTCTGGGATGACCCATACCCTGAGGTC CTGAGCCAGGAGCCCTTCCACAAGGTGACGGTGCAGAGCCTGCTGACTGTTGAGACC TTAGAGCACAACCAAACCTACGAGTGCAGGGCCCACAACAGCGTGGGGAGTGGCTCC TGGGCCTTCATACCCATCTCTGCAGGAGCCCACACGCATCCCCCGGATGAGTTCCTCT TCACACCAGTGGTGGTCGCCTGCATGTCCATCATGGCCTTGCTGCTGCTGCTGCTCCT GCTGCTATTGTACAAGTATAAGCAGAAGCCCAAGTACCAGGTCCGCTGGAAGATCATC GAGAGCTATGAGGGCAACAGTTATACTTTCATCGACCCCACGCAGCTGCCTTACAACG AGAAGTGGGAGTTCCCCCGGAACAACCTGCAGTTTGGTAAGACCCTCGGAGCTGGAG CCTTTGGGAAGGTGGTGGAGGCCACGGCCTTTGGTCTGGGCAAGGAGGATGCTGTCC TGAAGGTGGCTGTGAAGATGCTGAAGTCCACGGCCCATGCTGATGAGAAGGAGGCCC TCATGTCCGAGCTGAAGATCATGAGCCACCTGGGCCAGCACGAGAACATCGTCAACCT TCTGGGAGCCTGTACCCATGGAGGCCCTGTACTGGTCATCACGGAGTACTGTTGCTAT GGCGACCTGCTCAACTTTCTGCGAAGGAAGGCTGAGGCCATGCTGGGACCCAGCCTG AGCCCCGGCCAGGACCCCGAGGGAGGCGTCGACTATAAGAACATCCACCTCGAGAAG AAATATGTCCGCAGGGACAGTGGCTTCTCCAGCCAGGGTGTGGACACCTATGTGGAGA TGAGGCCTGTCTCCACTTCTTCAAATGACTCCTTCTCTGAGCAAGACCTGGACAAGGAG GATGGACGGCCCCTGGAGCTCCGGGACCTGCTTCACTTCTCCAGCCAAGTAGCCCAG GGCATGGCCTTCCTCGCTTCCAAGAATTGCATCCACCGGGACGTGGCAGCGCGTAAC GTGCTGTTGACCAATGGTCATGTGGCCAAGATTGGGGACTTCGGGCTGGCTAGGGAC ATCATGAATGACTCCAACTACATTGTCAAGGGCAATGCCCGCCTGCCTGTGAAGTGGA TGGCCCCAGAGAGCATCTTTGACTGTGTCTACACGGTTCAGAGCGACGTCTGGTCCTA TGGCATCCTCCTCTGGGAGATCTTCTCACTTGGGCTGAATCCCTACCCTGGCATCCTG GTGAACAGCAAGTTCTATAAACTGGTGAAGGATGGATACCAAATGGCCCAGCCTGCAT TTGCCCCAAAGAATATATACAGCATCATGCAGGCCTGCTGGGCCTTGGAGCCCACCCA CAGACCCACCTTCCAGCAGATCTGCTCCTTCCTTCAGGAGCAGGCCCAAGAGGACAGG AGAGAGCGGGACTATACCAATCTGCCGAGCAGCAGCAGAAGCGGTGGCAGCGGCAGC AGCAGCAGTGAGCTGGAGGAGGAGAGCTCTAGTGAGCACCTGACCTGCTGCGAGCAA GGGGATATCGCCCAGCCCTTGCTGCAGCCCAACAACTATCAGTTCTGCTGAGGAGTTG ACGACAGGGAGTACCACTCTCCCCTCCCACAAACTTCAACTCCTCCATGGATGGGGCG ACACGGGGAGAACATACAAACTCTGCCTTCGGTCATTTCACTCAACAGCTCGGCCCAG CTCTGAAACTTGGGAAGGTGAGGGATTCAGGGGAGGTCAGAGGATCCCACTTCCTGA GCATGGGCCATCACTGCCAGTCAGGGGCTGGGGGCTGAGCCCTCACCCCCCCCTCCC CTACTGTTCTCATGGTGTTGGCCTCGTGTTTGCTATGCCAACTAGTAGAACCTTCTTTC CTAATCCCCTTATCTTCATGGAAATGGACTGACTTTATGCCTATGAAGTCCCCAGGAGC TACACTGATACTGAGAAAACCAGGCTCTTTGGGGCTAGACAGACTGGCAGAGAGTGAG ATCTCCCTCTCTGAGAGGAGCAGCAGATGCTCACAGACCACACTCAGCTCAGGCCCCT TGGAGCAGGATGGCTCCTCTAAGAATCTCACAGGACCTCTTAGTCTCTGCCCTATACG CCGCCTTCACTCCACAGCCTCACCCCTCCCACCCCCATACTGGTACTGCTGTAATGAG CCAAGTGGCAGCTAAAAGTTGGGGGTGTTCTGCCCAGTCCCGTCATTCTGGGCTAGAA GGCAGGGGACCTTGGCATGTGGCTGGCCACACCAAGCAGGAAGCACAAACTCCCCCA AGCTGACTCATCCTAACTAACAGTCACGCCGTGGGATGTCTCTGTCCACATTAAACTAA CAGCATTAATGCA. (SEQ ID NO:298; NM_001288705.3), or a nucleic acid sequence that hybridizes to a nucleic acid sequence consisting of SEQ ID NO:298 under stringent hybridization conditions

In some embodiments, serum response factor (SRF, MCM1) comprises the amino acid sequence: MITSETGKALIQTCLNSPDSPPRSDPTTDQRMSATGFEETDLTYQVSESDSSGETKDTLKP AFTVTNLPGTTSTIQTAPSTSTTMQVSSGPSFPITNYLAPVSASVSPSAVSSANGTVLKSTG SGPVSSGGLMQLPTSFTLMPGGAVAQQVPVQAIQVHQAPQQASPSRDSSTDLTQTSSSG TVTLPATIMTSSVPTTVGGHMMYPSPHAVMYAPTSGLGDGSLTVLNAFSQAPSTMQVSHS QVQEPGGVPQVFLTASSGTVQIPVSAVQLHQMAVIGQQAGSSSNLTELQVVNLDTAHSTK SE. (SEQ ID NO:299; NP_001278930.1 or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:299)

In some embodiments, the nucleic acid sequence encoding SRF comprises the nucleic acid sequence:

(SEQ ID NO: 300; NM_001292001.2)
GCAGAGGTGGGAGTGACCGGCGCCAGAGAGGAAGAGGGCCCTTGCTGAG
TGAAGGGGCCTATGAGCTGTCCACGCTGACAGGGACACAGGTGCTGTTG
CTGGTGGCCAGTGAGACAGGCCATGTGTATACCTTTGCCACCCGAAAAC
TGCAGCCCATGATCACCAGTGAGACCGGCAAGGCACTGATTCAGACCTG
CCCCACAACAGACCAGAGAATGAGTGCCACTGGCTTTGAAGAGACAGAT
CCCTCAACTCGCCAGACTCTCCACCCCGTTCAGATCACCTACCAGGTGT
CGGAGTCTGACAGCAGTGGGGAGACCAAGGACACACTGAAGCCGGCGTT
CACAGTCACCAACCTGCCGGGTACAACCTCCACCATCCAAACAGCACCT
AGCACCTCTACCACCATGCAAGTCAGCAGCGGCCCCTCCTTTCCCATCA
CCAACTACCTGGCACCAGTGTCTGCTAGTGTCAGCCCCAGTGCTGTCAG
CAGTGCCAATGGGACTGTGCTGAAGAGTACAGGCAGCGGCCCTGTCTCC
TCTGGGGGCCTTATGCAGCTGCCTACCAGCTTCACCCTCATGCCTGGTG
GGGCAGTGGCCCAGCAGGTCCCAGTGCAGGCCATTCAAGTGCACCAGGC
CCCACAGCAAGCGTCTCCCTCCCGTGACAGCAGCACAGACCTCACGCAG
ACCTCCTCCAGCGGGACAGTGACGCTGCCCGCCACCATCATGACGTCAT
CCGTGCCCACAACTGTGGGTGGCCACATGATGTACCCTAGCCCGCATGC
GGTGATGTATGCCCCCACCTCGGGCCTGGGTGATGGCAGCCTCACCGTG
CTGAATGCCTTCTCCCAGGCACCATCCACCATGCAGGTGTCACACAGCC
AGGTCCAGGAGCCAGGTGGCGTCCCCCAGGTGTTCCTGACAGCATCATC
TGGGACAGTGCAGATCCCTGTTTCAGCAGTTCAGCTCCACCAGATGGCT
GTGATAGGGCAGCAGGCCGGGAGCAGCAGCAACCTCACCGAGCTACAGG
TGGTGAACCTGGACACCGCCCACAGCACCAAGAGTGAATGATCCGCCCG
CCGCCCTGGACAGATGGCCCAAGGGATGGCACCACTTATTTATTGTTGC
CTTTTCACGTTTTCTTTACACACACGTTGACGGGCCGCAGGAGGGAGGC
GGGGAGGAGGAACGGGCAGCCACAGGACTGAGCCCTCTCACTCCAGCCA
AAGAAATGGGCCTGCCTGCCTCCACCCGTCCTCCCTCAGCCTCCCCTTC
TTCCCGCCCCACCTCCCATTTCTGTTGCTGGAGGGGCTGTCCTCCTTCC
TGGGACCCCCTCGCCAGCTTGGCTCGATGTTTGCCATGAGTATTAGCTT
ACCCAATGGGACCGTGCCCCACCTCCCCACACACAGGCCTTCTGTGGGG
CTGGGCACCGTGTCCTCCTCTGAGGAAGCAGTTGGGGCCCTCTTGCCAG
CCTCCTTGCTGACCCCAGGTCAGCCCTGTGTCTGTCACAGGCTGGGTCA
AAAGAGCCCTGGCTCTGCCCCTCAGGGGGCCAGCTGGGGAGATGGGGGC
TTCTTCCTCACACTGCTGTCCTCTCCCCCTTCAGCTCCTGAGTAGCTGG
GCCTGTGCACTGGGCAGGTTCCTGGGGCCGCCTGCCCTGCCTTGCCGCT
CCCCTTGGACCTCCAGGGGCTCCTGGGTTGGAGGGAACCACCAGCGTTC
CCTTCTCCCCCTTGTCTTCCCCCCTCTCCTCCCAGCTGCTTTACTTAAA
GTTGATTTTGAACTTTTTATTTGAGGAGACGAAGTGAAAACAAATCTAT
AAATATATATTTTTAAAATATTTAACTTTTTTTTATGGCGTTTTTCTCG
TCCCCCTCCCTGCCCAAACTCCCCTTCCCTGGGGAGCCCTCAGGCTCCC
CAGAACTGGCTGGGCCCCTGGGGACAGAGCCACCCCATGAGCTCGGGGT
CCACCAGTGTGTGGGGGAGATTCTGGGTTTGCCCAGTCCTGGGTTGTTT
CCAGGAGAAAGCCGGGGGAGGGGCCCTCAGGCCATTCCCCAACGGGGTG
GGGAGGGTGACCCACAGCTCTGGGCCTCTTTTTGCCCTTTAGGGCTGTT
GCTAGGGAGAGGGAAGAGGGAGACCAAATGTCGGGGTTGGGGTGGGAGG
GCGTCAGGCAGAGGCAACTGACTTCATTTGTGCCACACGCATGGGCATT
GCAGCCTTGCGCTGTCCCAGGCATGCAGCTGCCTGGGGCCCAAGTTGCA
GTGAGCAGGGTGGGGTCTGGGAGGGGGTGAGAGGCAGGAATGGGGGTCA
GAAGAAGTGGGAGCAGCTTCTTGGGCTGAGTGCAGCCAAAGGGGAGCCA
GAAATGGGCAGTTCTCCCAGGGAGTGAGCAGCTACTGTAACTTTTTTAA
ATTAAGACAAAAAGCCTTGAAGAAAATGACTTTATTTTTCTAAGTGTAA
CCTCAGTATTTATGTAATTTGTACAGGGGCCATGCCCCACCCCCCTCCT
CCCCCTTTGGGGTAGACCTTGAGGGTGGGCCAGCATAGGGGGGAGGGTC
TTTTACCCTGTGTCAGAGCCTACCTTCACCACCTATATCCAGAAGGGGA
GCTTTTTCAGAAACAGGGCAGCAGTGGGGTGAAATTTTCTTAACCCCTA
AGACTGCCTTCAGTAGGAACAAGCTGGCTTCTGTGATTAGGTGAAGGGA
TGGGGGAAGATTTTATGCACAGCCTAGTTATCAAGGGGATGATTTGCCG
ACATGTTTGAGAACCCCCTAACCTCTAACCCTCATTGCTGTCTTGCCCC
AGTTTGGGGTGCCAAGATGGAAGTCACCTTTCTGGGCTTTCTCCTGGAG
ATAGCTGGGGCTTATGGGTGGCTTTCAAGGCTGGGGCATGGCAAATCAG
GGGCCAGAGAGCAGGGGAGCTTGGGACTCAGGTCTGTAACTGCCCAGCC
CCTTTTCTCTGCTCTTGTTTCACTCCACCATCACTCACTCACTCCCCAC
TCCCCCACCCATGGGGAGGAGACCTTTGATGAATTCTTCCTCTCCTTCC
CACAAAAGACAGACCCAGTGAGTGAATCAGGCAAAGTGCTTATAATGTG
TGTTGTGTGAGCGTGGCCTTGGGAGGACATGCGTGTGTCAGGGATGAGT
TGAGGTGATATTTTTATGTGCAGCGACCCTTGGTGTTTCCCTTCCTCGG
TGGCTCTGGGGTATGTGTGTGTGGGTGTGTGCGCCTGAGTGAGTGTGTG
TGCTTGAATGTGAGTGTGTATGTCAGTGGTTTCTACTTCCCCTGGGATG
CTGACCCAGGAATAGTGGACATGGTCACAGTCCTATGTACAGAGCTTTC
TTTTGTATTAAAAAAAAATACTCTTTCAATAAATGTATCATTTTTGTGC
ACAGA.,

or a nucleic acid sequence that hybridizes to a nucleic acid sequence consisting of SEQ ID NO:300 under stringent hybridization conditions

In some embodiments, GLI family zinc finger 2 (GLI2, CJS, HPE9, PHS2, THP1, THP2) comprises the amino acid sequence: METSASATASEKQEAKSGILEAAGFPDPGKKASPLVVAAAAAAAVAAQGVPQHLLPPFHAP LPIDMRHQEGRYHYEPHSVHGVHGPPALSGSPVISDISLIRLSPHPAGPGESPFNAPHPYV NPHMEHYLRSVHSSPTLSMISAARGLSPADVAQEHLKERGLFGLPAPGTTPSDYYHQMTL VAGHPAPYGDLLMQSGGAASAPHLHDYLNPVDVSRFSSPRVTPRLSRKRALSISPLSDASL DLQRMIRTSPNSLVAYINNSRSSSAASGSYGHLSAGALSPAFTFPHPINPVAYQQILSQQRG LGSAFGHTPPLIQPSPTFLAQQPMALTSINATPTQLSSSSNCLSDTNQNKQSSESAVSSTVN PVAIHKRSKVKTEPEGLRPASPLALTQGQVSGHGSCGCALPLSQEQLADLKEDLDRDDCK QEAEVVIYETNCHWEDCTKEYDTQEQLVHHINNEHIHGEKKEFVCRWQACTREQKPFKAQ YMLVVHMRRHTGEKPHKCTFEGCSKAYSRLENLKTHLRSHTGEKPYVCEHEGCNKAFSN ASDRAKHQNRTHSNEKPYICKIPGCTKRYTDPSSLRKHVKTVHGPDAHVTKKQRNDVHLR TPLLKENGDSEAGTEPGGPESTEASSTSQAVEDCLHVRAIKTESSGLCQSSPGAQSSCSS EPSPLGSAPNNDSGVEMPGTGPGSLGDLTALDDTPPGADTSALAAPSAGGLQLRKHMTT MHRFEQLKKEKLKSLKDSCSWAGPTPHTRNTKLPPLPGSGSILENFSGSGGGGPAGLLPN PRLSELSASEVTMLSQLQERRDSSTSTVSSAYTVSRRSSGISPYFSSRRSSEASPLGAGRP HNASSADSYDPISTDASRRSSEASQCSGGSGLLNLTPAQQYSLRAKYAAATGGPPPTPLP GLERMSLRTRLALLDAPERTLPAGCPRPLGPRRGSDGPTYGHGHAGAAPAFPHEAPGGG ARRASDPVRRPDALSLPRVQRFHSTHNVNPGPLPPCADRRGLRLQSHPSTDGGLARGAY SPRPPSISENVAMEAVAAGVDGAGPEADLGLPEDDLVLPDDVVQYIKAHASGALDEGTGQ VYPTESTGFSDNPRLPSPGLHGQRRMVAADSNVGPSAPMLGGCQLGFGAPSSLNKNNMP VQWNEVSSGTVDALASQVKPPPFPQGNLAVVQQKPAFGQYPGYSPQGLQASPGGLDST QPHLQPRSGAPSQGIPRVNYMQQLRQPVAGSQCPGMTTTMSPHACYGQVHPQLSPSTIS GALNQFPQSCSNMPAKPGHLGHPQQTEVAPDPTTMGNRHRELGVPDSALAGVPPPHPVQ SYPQQSHHLAASMSQEGYHQVPSLLPARQPGFMEPQTGPMGVATAGFGLVQPRPPLEPS PTGRHRGVRAVQQQLAYARATGHAMAAMPSSQETAEAVPKGAMGNMGSVPPQPPPQDA GGAPDHSMLYYYGQIHMYEQDGGLENLGSCQVMRSQPPQPQACQDSIQPQPLPSPGVN QVSSTVDSQLLEAPQIDFDAIMDDGDHSSLFSGALSPSLLHSLSQNSSRLTTPRNSLTLPSIP AGISNMAVGDMSSMLTSLAEESKFLNMMT. (SEQ ID NO:301; NP_001358200.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:301)

In some embodiments, the nucleic acid sequence encoding GLI2 comprises the nucleic acid sequence: GCTGATGGATTGCAGAAGTGCCGGCGCTTGCCAGCCGAGGCAGCACGGCTCCGCGG ACTTTTTTTCAAACTCCCATCAATGAGACTTCGAGGAGGAGCGGGCGGCGGCGGCGG CTGCGACTGCGAACGCGGAGGAAGGCCAGGAGCCGCAGGAGGAGCCGGAGGAAAGA GCTTGGGCCGCGCGGCGCGCCGCAGCCTCGGGGAGCCGCCTGCTCGCCGGCGGTA GGGGCTGCGCGGCGCCCGCCCGCCTCTCGGTCCCCTCTCTTGCCTGGCCCGCCCCG CCCCGGCTGGCTGGAGCCCCGGCACAAGGCAGCCAGCCGAGGGTCGCCGCGCCAGC CAAGGTGGGATGGGGGCCCACAGCCACCGCCCGGCGCCCGAGAGGCCACCTGCGTG CTAGAGGCAAACTTTTGTCTCTCTCGGATTGCCACCCAGGACGATGAGCGGCTGAGAT GGAGACGTCTGCCTCAGCCACTGCCTCCGAGAAGCAAGAAGCCAAAAGTGGGATCCT GGAGGCCGCTGGCTTCCCCGACCCGGGTAAAAAGGCCTCTCCTTTGGTGGTGGCTGC AGCGGCAGCAGCAGCGGTAGCTGCCCAAGGAGTGCCGCAGCATCTCTTGCCACCATT CCATGCGCCCCTACCGATTGACATGCGACACCAGGAAGGAAGGTACCATTACGAGCCT CATTCTGTCCACGGTGTGCACGGGCCCCCTGCCCTCAGCGGCAGCCCTGTCATCTCT GACATCTCCTTGATCCGGCTTTCCCCGCACCCGGCTGGCCCTGGGGAGTCCCCCTTCA ACGCCCCCCACCCGTACGTGAACCCCCACATGGAGCACTACCTCCGTTCTGTGCACAG CAGCCCCACGCTCTCCATGATCTCTGCAGCCAGGGGCCTCAGCCCCGCTGATGTGGC CCAGGAGCACCTTAAGGAGAGGGGACTGTTTGGCCTTCCTGCTCCAGGCACCACCCC CTCAGACTATTACCACCAGATGACCCTCGTGGCAGGCCACCCCGCGCCCTACGGGGA CCTGCTGATGCAGAGCGGGGGCGCTGCCAGCGCACCCCATCTCCACGACTACCTCAA CCCCGTGGACGTGTCCCGTTTCTCCAGCCCGCGGGTGACGCCCCGCCTGAGCCGCAA GCGGGCGCTGTCCATCTCCCCACTCTCAGACGCCAGCCTGGACCTGCAGCGGATGAT CCGCACCTCACCCAACTCGCTAGTGGCCTACATCAACAACTCCCGAAGCAGCTCGGCG GCCAGCGGTTCCTACGGGCATCTGTCAGCGGGTGCCCTCAGCCCAGCCTTCACCTTC CCCCACCCCATCAACCCCGTGGCCTACCAGCAGATTCTGAGCCAGCAGAGGGGTCTG GGGTCAGCCTTTGGACACACACCACCCCTGATCCAGCCCTCACCCACCTTCCTGGCCC AGCAGCCCATGGCCCTCACCTCCATCAATGCCACGCCCACCCAGCTCAGCAGCAGCA GCAACTGTCTGAGTGACACCAACCAGAACAAGCAGAGCAGTGAGTCGGCCGTCAGCA GCACCGTCAACCCTGTCGCCATTCACAAGCGCAGCAAGGTCAAGACCGAGCCTGAGG GCCTGCGGCCGGCCTCCCCTCTGGCGCTGACGCAGGGCCAGGTGTCTGGACACGGC TCATGTGGGTGTGCCCTTCCCCTCTCCCAGGAGCAGCTGGCTGACCTCAAGGAAGATC TGGACAGGGATGACTGTAAGCAGGAGGCTGAGGTGGTCATCTATGAGACCAACTGCCA CTGGGAAGACTGCACCAAGGAGTACGACACCCAGGAGCAGCTGGTGCATCACATCAA CAACGAGCACATCCACGGGGAGAAGAAGGAGTTTGTGTGCCGCTGGCAGGCCTGCAC GCGGGAGCAGAAGCCCTTCAAGGCGCAGTACATGCTGGTGGTGCACATGCGGCGACA CACGGGCGAGAAGCCCCACAAGTGCACGTTCGAGGGCTGCTCGAAGGCCTACTCCCG CCTGGAGAACCTGAAGACACACCTGCGGTCCCACACCGGGGAGAAGCCATATGTGTG TGAGCACGAGGGCTGCAACAAAGCCTTCTCCAACGCCTCGGACCGCGCCAAGCACCA GAATCGCACCCACTCCAACGAGAAACCCTACATCTGCAAGATCCCAGGCTGCACCAAG AGATACACAGACCCCAGCTCTCTCCGGAAGCATGTGAAAACGGTCCACGGCCCAGATG CCCACGTCACCAAGAAGCAGCGCAATGACGTGCACCTCCGCACACCGCTGCTCAAAG AGAATGGGGACAGTGAGGCCGGCACGGAGCCTGGCGGCCCAGAGAGCACCGAGGCC AGCAGCACCAGCCAGGCCGTGGAGGACTGCCTGCACGTCAGAGCCATCAAGACCGAG AGCTCCGGGCTGTGTCAGTCCAGCCCCGGGGCCCAGTCGTCCTGCAGCAGCGAGCC CTCTCCTCTGGGCAGTGCCCCCAACAATGACAGTGGCGTGGAGATGCCGGGGACGGG GCCCGGGAGCCTGGGAGACCTGACGGCACTGGATGACACACCCCCAGGGGCCGACA CCTCAGCCCTGGCTGCCCCCTCCGCTGGTGGCCTCCAGCTGCGCAAACACATGACCA CCATGCACCGGTTCGAGCAGCTCAAGAAGGAGAAGCTCAAGTCACTCAAGGATTCCTG CTCATGGGCCGGGCCGACTCCACACACGCGGAACACCAAGCTGCCTCCCCTCCCGGG AAGTGGCTCCATCCTGGAAAACTTCAGTGGCAGTGGGGGCGGCGGGCCCGCGGGGC TGCTGCCGAACCCGCGGCTGTCGGAGCTGTCCGCGAGCGAGGTGACCATGCTGAGCC AGCTGCAGGAGCGCCGCGACAGCTCCACCAGCACGGTCAGCTCGGCCTACACCGTGA GCCGCCGCTCCTCCGGCATCTCCCCCTACTTCTCCAGCCGCCGCTCCAGCGAGGCCT CGCCCCTGGGCGCCGGCCGCCCGCACAACGCGAGCTCCGCTGACTCCTACGACCCC ATCTCCACGGACGCGTCGCGGCGCTCGAGCGAGGCCAGCCAGTGCAGCGGCGGCTC CGGGCTGCTCAACCTCACGCCGGCGCAGCAGTACAGCCTGCGGGCCAAGTACGCGG CAGCCACTGGCGGCCCCCCGCCCACTCCGCTGCCGGGCCTGGAGCGCATGAGCCTG CGGACCAGGCTGGCGCTGCTGGACGCGCCCGAGCGCACGCTGCCCGCCGGCTGCCC ACGCCCACTGGGGCCGCGGCGTGGCAGCGACGGGCCGACCTATGGCCACGGCCACG CGGGGGCTGCGCCCGCCTTCCCCCACGAGGCTCCAGGCGGCGGAGCCAGGCGGGC CAGCGACCCTGTGCGGCGGCCCGATGCCCTGTCCCTGCCGCGGGTGCAGCGCTTCC ACAGCACCCACAACGTGAACCCCGGCCCGCTGCCGCCCTGTGCCGACAGGCGAGGC CTCCGCCTGCAGAGCCACCCGAGCACCGACGGCGGCCTGGCCCGCGGCGCCTACTC GCCCCGGCCGCCTAGCATCAGCGAGAACGTGGCGATGGAGGCCGTGGCGGCAGGAG TGGACGGCGCGGGGCCCGAGGCCGACCTGGGGCTGCCGGAGGACGACCTGGTGCTT CCAGACGACGTGGTGCAGTACATCAAGGCGCACGCCAGTGGCGCTCTGGACGAGGGC ACCGGGCAGGTGTATCCCACGGAAAGCACTGGCTTCTCTGACAACCCCAGACTACCCA GCCCGGGGCTGCACGGCCAGCGCAGGATGGTGGCTGCGGACTCCAACGTGGGCCCC TCCGCCCCTATGCTGGGAGGATGCCAGTTAGGCTTTGGGGCGCCCTCCAGCCTGAAC AAAAATAACATGCCTGTGCAGTGGAATGAGGTGAGCTCCGGCACCGTAGACGCCCTGG CCAGCCAGGTGAAGCCTCCACCCTTTCCTCAGGGCAACCTGGCGGTGGTGCAGCAGA AGCCTGCCTTTGGCCAGTACCCGGGCTACAGTCCGCAAGGCCTACAGGCTAGCCCTG GGGGCCTGGACAGCACGCAGCCACACCTGCAGCCCCGCAGCGGAGCCCCCTCCCAG GGCATCCCCAGGGTAAACTACATGCAGCAGCTGCGACAGCCAGTGGCAGGCAGCCAG TGTCCTGGCATGACTACCACTATGAGCCCCCATGCCTGCTATGGCCAAGTCCACCCCC AGCTGAGCCCCAGCACCATCAGTGGGGCCCTCAACCAGTTCCCCCAATCCTGCAGCAA CATGCCAGCCAAGCCAGGGCATCTGGGGCACCCTCAGCAGACAGAAGTGGCACCTGA CCCCACCACGATGGGCAATCGCCACAGGGAACTTGGGGTCCCCGATTCAGCCCTGGC TGGAGTGCCACCACCTCACCCAGTCCAGAGCTACCCACAGCAGAGCCATCACCTGGC AGCCTCCATGAGCCAGGAGGGCTACCACCAGGTCCCCAGCCTTCTGCCTGCCCGCCA GCCTGGCTTCATGGAGCCCCAAACAGGCCCGATGGGGGTGGCTACAGCAGGCTTTGG CCTAGTGCAGCCCCGGCCTCCCCTCGAGCCCAGCCCCACTGGCCGCCACCGTGGGG TACGTGCTGTGCAGCAGCAGCTGGCCTACGCCAGGGCCACAGGCCATGCCATGGCTG CCATGCCGTCCAGTCAGGAAACAGCAGAGGCTGTGCCCAAGGGAGCGATGGGCAACA TGGGGTCGGTGCCTCCCCAGCCGCCTCCGCAGGACGCAGGTGGGGCCCCGGACCAC AGCATGCTCTACTACTACGGCCAGATCCACATGTACGAACAGGATGGAGGCCTGGAGA ACCTCGGGAGCTGCCAGGTCATGCGGTCCCAGCCACCACAGCCACAGGCCTGTCAGG ACAGCATCCAGCCCCAGCCCTTGCCCTCACCAGGGGTCAACCAGGTGTCCAGCACTG TGGACTCCCAGCTCCTGGAGGCCCCCCAGATTGACTTCGATGCCATCATGGATGATGG CGATCACTCGAGTTTGTTCTCGGGTGCTCTGAGCCCCAGCCTCCTCCACAGCCTCTCC CAGAACTCCTCCCGCCTCACCACCCCCCGAAACTCCTTGACCCTGCCCTCCATCCCCG CAGGCATCAGCAACATGGCTGTCGGGGACATGAGCTCCATGCTCACCAGCCTCGCCG AGGAGAGCAAGTTCCTGAACATGATGACCTAGAGGCCCGAGCGCCTGGTGCTGAGTG CACCCGGAGGGGTCATCGCTGCCCAGAGCCTGGGGATTCCAGCTGTCTTGTCTTTTTC CAAAAAAGTGTTAAATAGGCTTGAGGGGTTGTTGCGCAATGGCCGCTTCAGATGACAG ATGTTGTAAGAGAAGGTTTATGGGCATCCTCTCTGGTCTTTTGGATTATTCCTCAGAACA ATGAAAAAAGTCTCCATAGGACAGGAAGGAATGCAAAACTCATTTACACAGTGCTTTCC AGCCTTTGGTGCTTACAGGACCGCGCTGTTCCGGCTTCTTCACGGCTGACATTCGGCT AACGAGGGATTACTTTGGCCAAAACCTTTCAAAGGATATGCAGAAAGATGGTAGGGAG CATTTGGGTTTGAATCTGAATGCTATACTGGATACTCTGCTCCGGAAAGATGAGCTTTTT ATTCTACTACTTGGAAGGAAAAGGAATTCCTGGTCCACCTGAATTCCTCTATGAAGCCT AACTCTTGAGGTCTCTAACATACCTTGTCATAGAGGAAAAGCACAGATTATACCTGGAT GATTCAGGAGCACATTCTGATTCCAGGTTTGGTAGAGCTGGCTCTTCTACTCCGTAAAG CCGAGTCTGGGACTGGCAGCCCATCCAAGTGTATATGAATGAATAAAGCATCCAAGTAT ATATGAATGAATAAAGTATGTAAGTATCACCAGAAAAAGGAAAGAAAAAATGTACTCCTT GGGGCAAGCCCAGAAGCTGCCCTGGCCTCTCCAGACCGTGTTTACAGTGTTTGCATGT AGAATGTAGCCCTTCCTGAAAAGAAGACTTGTTTCTAAATACCTCGGGGCTGCTGGAGC CGCTGTGGGTTAGGGATGGACTGAGGCCTCGAGGAGTGAGGGTGCACCCGGGGCCC AGCCTCAGGCTGCCCTAGGGATCTCTCAGTAGGAAGAGGAAGTTGCGTGTTTACCCAA TCCTGTTTCTCCAATGCAACGTCCACCCACTTTACCACCAAAAACTCCAGGGCCTGACG GCAGCCCGGTCCCCCAGCACTCACCAGCAGCCCAGTGTTCTCCACCAAGCCACAGTG TGCATGCCTGGTATCCTCCGGATTCCCTTCCTTCTGCCCGCTGAGTCACTGGGCAGAG AATGATGACATGTGTAGGTGGTGTGGTTGGGGGTGGAAAGGGGAAGGGGTTGATCCT CAGGACTCTGAGGGAGCATCGTTGAATTTTCCTGTTCAGTGTGACCAAGACCCACCTG GAAATGGAATTTGGAACTGGCTTCAGGAGACATCATTCCTGAACACACTGTAGGGTGAA TTGGTGCATCTTCCCCACCATACACACACACACACACACACACACACACACACACACAC ACACACCCCAAACCTTTTCATGGGGAATGTGTGGCAACCTTGCCAAACAGCACCACTC AGAGTGTGACTCTGACTGTGACCTTGGCCTTAATGAGGAACTTCTTAGGAGAGTTTGAG GACAAGGCCAACATCGTCATCTGGGCTCGCTGCGTCCCAGCACATCAAACTCTGTCCA GAGACAAGGCCAACTGCAAATGAAAGCCAGGGAACATTGCTAAGGGTCTGTGGCTCTG TGGTGGTGTTCATCGCCTTCCTGAGATAGGATTTCCCTTGCCAGTCCCAACCTGTATAT ATTCTGTACAGAAGACATCCCTGAATATACTGTAGGTGAGTCGTCCAGCCAAATTTATAT CTCCAAAACATTTTTAGCTTTTTCTACATGCTATGAATTGAGATGACATGCTCAACTTGT AAATAAGTCTTTTTGTACATTAAAAAAGTAATTTTTTCATAATTTATCTTGTCTATCTGCTT CCCCCTTGACAGTAGTTAATGAGAACCTGGGCAGTAAATTTGGTGCATTCGAGCAGAAA TTAGGCTGTATTTTTTCTTAACAGTGTCAAAATTGACTATCCCGCCTTTGCCAAGAAATG TTTAATGCTGAGGCA. (SEQ ID NO:302; NM_001371271.1), or a nucleic acid sequence that hybridizes to a nucleic acid sequence consisting of SEQ ID NO:302 under stringent hybridization conditions

In some embodiments, Sp4 transcription factor (SP4, HF1B, SPR-1) comprises the amino acid sequence: MATEGGKTSEPENNNKKPKTSGSQDSQPSPLALLAATCSKIGTPGENQATGQQQIIIDPSQ GLVQLQNQPQQLELVTTQLAGNAWQLVASTPPASKENNVSQPASSSSSSSSSNNGSASP TKTKSGNSSTPGQFQVIQVQNPSGSVQYQVIPQLQTVEGQQIQINPTSSSSLQDLQGQIQLI SAGNNQAILTAANRTASGNILAQNLANQTVPVQIRPGVSIPLQLQTLPGTQAQVVTTLPINIG GVTLALPVINNVAAGGGTGQVGQPAATADSGTSNGNQLVSTPTNTTTSASTMPESPSSST TCTTTASTSLTSSDTLVSSADTGQYASTSASSSERTIEESQTPAATESEAQSSSQLQPNGM QNAQDQSNSLQQVQIVGQPILQQIQIQQPQQQIIQAIPPQSFQLQSGQTIQTIQQQPLQNVQ LQAVNPTQVLIRAPTLTPSGQISWQTVQVQNIQSLSNLQVQNAGLSQQLTITPVSSSGGTTL AQIAPVAVAGAPITLNTAQLASVPNLQTVSVANLGAAGVQVQGVPVTITSVAGQQQGQDGV KVQQATIAPVTVAVGGIANATIGAVSPDQLTQVHLQQGQQTSDQEVQPGKRLRRVACSCP NCREGEGRGSNEPGKKKQHICHIEGCGKVYGKTSHLRAHLRWHTGERPFICNWMFCGKR FTRSDELQRHRRTHTGEKRFECPECSKRFMRSDHLSKHVKTHQNKKGGGTALAIVTSGEL DSSVTEVLGSPRIVTVAAISQDSNPATPNVSTNMEEF. (SEQ ID NO:303; NP_001313471.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:303)

In some embodiments, the nucleic acid sequence encoding SP4 comprises the nucleic acid sequence: ACAGCCCAGCGGCGGCCATTCGCGGAAAAAGAGGCAGAGCCTGTGCCAGCTACAGCC TCCTCCGAGCCACCGCGGGCGGGCGGGACCGGCCTCTCCTCCCGCCTCGCCCCCAC CCCCACCCACCTCTATCCCAGTGTCTCCGTCTGAGGGTTTGTCCTGTTAATGCGGGAT GAGCGAAGAAGGAGGAGGAGGAGGAGGCGGCAGCGGCAGCGGCGATGGCTACAGAA GGAGGGAAAACCTCTGAGCCAGAGAATAACAATAAAAAACCCAAAACCTCAGGCTCCC AGGACTCTCAGCCCTCTCCTCTGGCTTTACTGGCAGCTACTTGCAGCAAAATAGGGAC TCCTGGTGAAAATCAAGCAACTGGACAACAACAAATTATTATAGATCCAAGTCAAGGAT TGGTGCAACTTCAAAATCAACCACAACAGCTAGAACTGGTAACAACGCAACTTGCTGGA AACGCTTGGCAACTTGTTGCCTCCACTCCTCCTGCTTCAAAAGAGAATAACGTTTCTCA ACCAGCCTCTAGTTCGTCTAGTTCTTCCAGCAGTAATAACGGGAGTGCATCTCCTACAA AAACTAAATCAGGTAATTCTTCCACCCCTGGTCAATTTCAAGTCATACAAGTACAAAATC CAAGTGGTAGTGTACAGTACCAAGTAATTCCACAACTTCAGACAGTGGAAGGTCAACAA ATTCAAATCAATCCAACTAGTAGTTCATCTCTACAGGATTTGCAGGGTCAAATTCAGCTC ATTTCTGCAGGTAATAATCAAGCTATACTCACAGCTGCTAACAGGACAGCTTCTGGGAA TATTCTTGCTCAAAACCTGGCAAATCAGACAGTTCCGGTCCAAATTAGACCTGGTGTTT CAATACCACTGCAGTTACAGACTCTTCCTGGTACTCAGGCTCAAGTTGTAACAACCCTA CCAATTAACATTGGAGGAGTGACTCTAGCTTTGCCAGTGATAAACAACGTGGCTGCCG GAGGAGGGACTGGGCAGGTTGGCCAGCCTGCTGCTACTGCTGATAGTGGGACTTCCA ATGGGAATCAATTAGTTTCCACACCCACCAACACCACTACTTCTGCCAGTACTATGCCA GAATCTCCCTCCTCCTCCACTACCTGCACAACCACTGCTTCAACGTCTTTGACAAGCAG TGACACATTAGTGAGCTCAGCAGATACTGGCCAGTATGCAAGCACATCAGCCAGTAGT TCTGAACGCACCATTGAAGAATCTCAAACACCTGCTGCTACTGAGTCTGAAGCCCAGA GCTCCAGTCAGCTTCAGCCTAATGGAATGCAGAATGCACAGGATCAATCAAATTCTCTT CAGCAGGTGCAAATTGTAGGCCAACCTATCTTACAGCAGATCCAGATCCAACAGCCTC AGCAACAGATCATTCAGGCTATTCCACCACAGTCGTTTCAACTCCAGTCAGGGCAGAC GATTCAGACCATCCAGCAGCAGCCTTTACAGAATGTTCAACTTCAAGCAGTAAATCCGA CTCAGGTGCTTATCAGGGCTCCAACTTTAACACCTTCAGGGCAAATCAGTTGGCAAACT GTACAGGTTCAGAATATTCAGAGTCTTTCAAATTTGCAAGTTCAGAATGCTGGGTTATCC CAACAATTAACCATCACCCCAGTGTCTTCAAGTGGTGGCACAACTCTTGCTCAGATTGC TCCTGTGGCTGTTGCTGGTGCCCCAATAACTTTGAATACTGCCCAGCTTGCATCAGTGC CTAACCTTCAGACAGTGAGCGTTGCCAACCTGGGTGCTGCAGGTGTTCAAGTGCAGGG AGTTCCCGTTACAATCACTAGTGTTGCAGGTCAGCAGCAAGGACAAGATGGAGTAAAA GTCCAGCAAGCTACTATAGCTCCTGTAACTGTAGCAGTTGGAGGAATTGCTAATGCCAC GATAGGTGCTGTTAGTCCTGACCAACTCACACAAGTGCATTTGCAGCAAGGCCAGCAG ACTTCTGATCAAGAGGTACAACCTGGCAAGAGGCTTCGAAGAGTTGCCTGTTCCTGTC CTAATTGTAGGGAAGGAGAAGGAAGAGGCAGTAATGAACCAGGAAAAAAGAAGCAGCA TATCTGTCATATTGAAGGATGTGGTAAAGTTTATGGCAAAACATCTCATTTACGAGCACA TCTTCGCTGGCATACTGGAGAAAGACCTTTTATATGCAACTGGATGTTTTGTGGCAAAA GATTCACACGGAGTGATGAGCTCCAGAGACATAGAAGAACCCATACAGGTGAAAAGAG ATTTGAATGCCCGGAATGTTCTAAAAGGTTTATGCGGAGTGATCATCTCTCCAAACATG TCAAAACGCACCAGAATAAAAAAGGTGGTGGGACAGCTCTTGCCATTGTTACCTCGGG AGAACTGGACTCATCTGTTACAGAGGTGCTTGGCTCCCCAAGAATTGTCACAGTTGCA GCCATTTCTCAAGATTCGAATCCAGCAACTCCCAATGTTTCAACCAACATGGAAGAATT CTGAAAAGTTATTTATAACAGAGACCTCTAGTGCTGCACTTGTTTACACACCTTTGAAAA TCTGGAAATGGGCTGGTCAAGTGGATTACAGAGTAGGAAATTATGTTTTCATTCTTGGC TTCTTTAAGTATTCCAGGGTTTGGGGTCAACACGTGAAGTGTTGAATTTTAAAAAATACA AAAAGCAGACTGATGTACTGGAAACAGAAAAGTATTTCCTCCATACTATAAGTTGTAGTT GTTTGGAAATATATCACATAACCTTTATACAGAATCTTCCCATCTCTTAATATCATGTGTT AACATGTTTAAAAAGACCTTAGTAGTTTGCAGGCTGGACCTTAATTGGACTTATTTTCTT TGAAAGTACTTTGTTATAAATTCAGTCAGTAATAATTTACGTGTATTCTTTTTCTCTATAG CACAGAAAACAGATAGTTAACTGATGATAGGGATAATACTGTATTTCCTTAGCTTGATTT TTGGAAAATCAACCGAAAATAGTTTGGCCGTCTTTTCTAAATGTTAGAAATTCTTCAACA GTTGAATTAGGTAAGTTCCAAAACAGTAATCTGAGATGCATCTCAGATCTTTATTACCAC TACATTATAGTAGTGTGTATGCAGACAATCAGTGAAGTCCAATTACTTTCTCCATTTGGA GACACAAGAGGAACATAGAGTTAAATCTTAGGTTAAATTTTAGGTTGACACCTTAGGAA AATGCTGGGAAAAAAATGGTTAAAACAAAACTCATCATAGCTTCAGAAAAATAAAATGAG GCATCTTAACATGCAATGTTCTAAAGTTAGGATTGATTATATTCCTAACCCTAGGTTGAA CCACAAAATTTCATTTAAAATGTTTATATTTGGAAATATTTGCATAGAGTGTAAATTGTTC TGTAGTTTCATATTTTGTAAATATGAGTTATGTTGACAATGTGCAGAATTCTTTATGCTTT GATGTGGTAGCCAAAGAAAGAATTACACTTTTTTCCAAGGCCAGCAGAAAATTCTCTTTT AACTACATTGTAATTCTTGTTTTCCTCTACTAAAAATTGGCCAGTCCCATTTTATTTCTAG TGCTATGTAAGAAGGTAATTAGGAATTATAACACAGTAATGTTTTTATGTTACATCAATAA CTGAATTTTCCCTAAAAATTAGCCTAATATATAATAGATATATTATGAAGCAAAACTTTTA TTTTTGAAAAGGCAGAATAATTTTCAGTGAAGTAAGTGACTAAAGAAAAAAACTATATTA TTGTTTATGCAAGGGTCTTACAGGAAAGGGTCTTTTTTTTTTTTTTTTTGAGATGGAGTC TCGCTCTCTTGCCCAGGCTGGAGTGCAATGGCACGATCTCAGCTTACTGCAACCGCCG CCTCCCAGGTTCAAGCGATTCTCCTGTCTTAGCCTCCTGAGTAGCTGGGATTAACAGG CGCCTGCCACCATGCCTGGCTAATTTTTGTATTTTTAGTAGAGACAGGGTTTCGCCATG TTGGCCAGGCTGGTTTCAAACTCCTGACCTCAGGTGATCTGCCCACCTCGGCCTCCCA GAGTGCTGGGATTACAGGCATGAGCCACCATGCCCGGCTAGGAAAGGGTCTTACTAG GAAAGATGGCCAAAAGTTTCATATGAAAAAAATTGGATTATAAACCAGTAACTTAAATAT TAATAAGGATATTTTATGTTTTAAAAAAGTATTTACACAGAATCATAATCAGTGAAATTGA CCATTTGAAAACTAAAAGTTTTTACCTACCTGCTCAATTTATTAACATCATTGCTTTGGG GACTGTTTGAATATAGGTACGTGTTTTCTTGTGCATTCTCTATAATTTCAGGAAAAGTAC TAATGCGAATTCTCTTCCAAAATTGTGATGTTTCTTGTATTTTTGATGAAGGAGAAATACT GTAATGATCACTGTTTACACTATGTACACTTTAGGCCAGCCCTTTGTAGCGTTATATAAA ACTGAAGGTCTTTTGTGCTTTCAGTTTGTATAAAAAAGCTTTGAGATTAAAGGAAAAAAA AAATTTTTACACTGTGGTTATAAATTTCAAGTTTCTTAAAGCTTTTGTAGACTTGTAACAG AGTCTTTAAATTTAAGTTGGATTTTGTAAATTGTTTTGTATATTTTATTTAATGTACTCTTA ACAACTGATGTATCTGGCTTTAAAACATCAGAATTGGTTTGTTGTTTTGTTTGGTACAGA GGAGCATTTGGTAGTGTTCATTTTAATTTTATTATATAGGAGCTGAAACATCAAATATATA TTTTATCTATCATTATGCTACACAATCAGTGCTATAAATTTTTTTATGCAAAGAAACTTTC TTAATGTTTTAATCATGTTTCCTCAGAGTGACACTTTTTGTTGTTGTTAATACCAAGTATG AACACTCTGCATCATTATTCCATGAACCAGTTCTAATGCAAACCTATGTATGTCCATTAG AAATGGAAGTTATTTTTTAATCAACAATGAGGCCTATTATAAATTTATCAGATGAATCTAG ATAGCTTTATAGCATATAAAATATGTTAATTTGTGTTAGCAGGTGCACATTTCACCACTG AAATTAGAAATATTTTGACAGTCTGTTCTGCATACCATTCTGAGTCTACTTTTCTGTCTTT AGAAGAATCGTAAATTTCAGTGTCCTTTATTTGACTCAGTGGGATATAGCTGTTATAAGT AATAGGGCACAGATGTGCAGTAGAGTCTTGTTTAATGGCATTTCACTGTTCATTCCCTTT ACCACCGTTATAAAACTTTTCTTTATTGTAATTATCAGTGCAAAGCTATGTATTTATCATG GTAAAACTCCAGTGTTAGAATAGTTTTTTCTTACAGTATACTTTCTTTGGTTAGGTTTGTG TATGTGTTGCTGATTACATTAGAACTTGATGTTAAGTCATTTATCACACTCTCATGAGAG CAGTAATAAAAGTGTGTAATCTAGGAGAAAAAGTTAATTTGTCAAACTTAGATAAGCATG ATGTTTAGGTCCTATTTTTCAATTTTATAACTGTTTTATTGCAACAATATTTGTATTTAAGT CTCCATTTTAATGCCTTGTGGTGTTTTTTTATGCATGTCACTAAGTTGTCATCCCACATA AATTGATGTGCAGCATAGGGTATTAAATCTACATAATGATTTTAAAACAGAAATAGTTGA TGGTAAAATGTAAATGTTTTGCAAAAATTCCTTATAAAAAGTTTTGTAGTAACATTTCACT TGTAAATTTTTTTTGTAAAAAAAAAAAAATGAAAAAAAAAGATGAATCCAGAAAAAAACCT GTTTCCCATATTCTAGAATTTAGACAATTATTCTGCCAGCAAAGCCTCTGGGGCTGTAAT TGACATTTTTACAGTGCTGATTTGTATAAAATTTGTTTTTTGTGGATTTGGAAATAAAATC ATGTACAAGTTGTTGCCTGCAATAACAATTGCAAGTAACCTATTAAAAATTCCCTTGAGT TTAACATGTTTCATTTAATTATGTATACTATAAAGCAGCAATAAATTATTTGAACTATCAA CCTA. (SEQ ID NO:304; NM_001326542.2), or a nucleic acid sequence that hybridizes to a nucleic acid sequence consisting of SEQ ID NO:304 under stringent hybridization conditions

In some embodiments, activating transcription factor 2 (ATF2, HB16, CREB2, TREB7, CRE-BP1) comprises the amino acid sequence: MKFKLHVNSARQYKDLWNMSDDKPFLCTAPGCGQRFTNEDHLAVHKHKHEMTLKFGPAR NDSVIVADQTPTPTRFLKNCEEVGLFNELASPFENEFKKASEDDIKKMPLDLSPLATPIIRSKI EEPSVVETTHQDSPLPHPESTTSDEKEVPLAQTAQPTSAIVRPASLQVPNVLLTSSDSSVIIQ QAVPSPTSSTVITQAPSSNRPIVPVPGPFPLLLHLPNGQTMPVAIPASITSSNVHVPAAVPLV RPVTMVPSVPGIPGPSSPQPVQSEAKMRLKAALTQQHPPVTNGDTVKGHGSGLVRTQSE ESRPQSLQQPATSTTETPASPAHTTPQTQSTSGRRRRAANEDPDEKRRKFLERNRAAASR CRQKRKVWVQSLEKKAEDLSSLNGQLQSEVTLLRNEVAQLKQLLLAHKDCPVTAMQKKSG YHTADKDDSSEDISVPSSPHTEAIQHSSVSTSNGVSSTSKAEAVATSVLTQMADQSTEPAL SQIVMAPSSQSQPSGS. (SEQ ID NO:305; NP_001243019.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:305)

In some embodiments, the nucleic acid sequence encoding ATF2 comprises the nucleic acid sequence: GTCAGTCCGATCTCGCGAGAGAGGACGGAAGCCTGTGGGAGCCCGTGGCCTTTAAAG TGCCGTTCAGCCTTTTCCTCCAGGGGTGCTTTGTAAACACGGCTGTGCTCAGGGCTCG CGGGTGACCGAAAGGATCATGAACTAGTGACCTGGAAAGGGTACTAGATGGAAACTTG AGAAAGGACTGCTTATTGATAACAGCTAAGGTATTCCTGGAAGCAGAGTAAATAAAGCT CATGGCCCACCAGCTAGAAAGATTTCCTGAAGAGGTAGTCTGATTGGCTTAACTCGTAT TCTTGCCATGAGAAAAAGAATGTGATAAGTTATTCAACTTATGAAATTCAAGTTACATGT GAATTCTGCCAGGCAATACAAGGACCTGTGGAATATGAGTGATGACAAACCCTTTCTAT GTACTGCGCCTGGATGTGGCCAGCGTTTTACCAACGAGGATCATTTGGCTGTCCATAA ACATAAACATGAGATGACACTGAAATTTGGTCCAGCACGTAATGACAGTGTCATTGTGG CTGATCAGACCCCAACACCAACAAGATTCTTGAAAAACTGTGAAGAAGTGGGTTTGTTT AATGAGTTGGCGAGTCCATTTGAGAATGAATTCAAGAAAGCTTCAGAAGATGACATTAA AAAAATGCCTCTAGATTTATCCCCTCTTGCAACACCTATCATAAGAAGCAAAATTGAGGA GCCTTCTGTTGTAGAAACAACTCACCAGGATAGTCCTTTACCTCACCCAGAGTCTACTA CCAGTGATGAGAAGGAAGTACCATTGGCACAAACTGCACAGCCCACATCAGCTATTGT TCGTCCAGCATCATTACAGGTTCCCAATGTGCTGCTTACAAGTTCTGACTCAAGTGTAA TTATTCAGCAGGCAGTACCTTCACCAACCTCAAGTACTGTAATCACCCAGGCACCATCC TCTAACAGGCCAATTGTCCCTGTACCAGGCCCATTTCCTCTTCTGTTACATCTTCCTAAT GGACAAACCATGCCTGTTGCTATTCCTGCATCAATTACAAGTTCTAATGTGCATGTTCCA GCTGCAGTCCCACTCGTTCGACCAGTCACCATGGTGCCTAGTGTTCCAGGAATCCCAG GTCCTTCCTCTCCCCAACCAGTACAGTCAGAAGCAAAAATGAGATTAAAAGCTGCTTTG ACCCAGCAACATCCTCCAGTTACCAATGGTGATACTGTCAAAGGTCATGGTAGCGGATT GGTTAGGACTCAGTCAGAGGAATCTCGACCGCAGTCATTACAACAGCCAGCCACATCC ACTACAGAAACTCCGGCTTCTCCAGCTCACACAACTCCACAGACCCAAAGTACAAGTG GTCGTCGGAGAAGAGCAGCTAACGAAGATCCTGATGAAAAAAGGAGAAAGTTTTTAGA GCGAAATAGAGCAGCAGCTTCAAGATGCCGACAAAAAAGGAAAGTCTGGGTTCAGTCT TTAGAGAAGAAAGCTGAAGACTTGAGTTCATTAAATGGTCAGCTGCAGAGTGAAGTCAC CCTGCTGAGAAATGAAGTGGCACAGCTGAAACAGCTTCTTCTGGCTCATAAAGATTGCC CTGTAACCGCCATGCAGAAGAAATCTGGCTATCATACTGCTGATAAAGATGATAGTTCA GAAGACATTTCAGTGCCGAGTAGTCCACATACAGAAGCTATACAGCATAGTTCGGTCAG CACATCCAATGGAGTCAGTTCAACCTCCAAGGCAGAAGCTGTAGCCACTTCAGTCCTC ACCCAGATGGCGGACCAGAGTACAGAGCCTGCTCTTTCACAGATCGTTATGGCTCCTT CCTCCCAGTCACAGCCCTCAGGAAGTTGATTAAAAACCTGCAGTACAACAGTTTTAGAT ACTCATTAGTGACTTCAAAGGGAAATCAAGGAAAGACCAGTTTCCATTTATGCGAAATC TGTGGTTGTAAATTTTTTTTTTTTACTTGAAATTAAATTTGGCTCTAAAGTTGGTGTAGCA GCAGTTGATCAGACTGAAAAACGGTTTTTAGTCTCTGGAAAAAGACTGATTTTGCTTTTT TTATAAATATTATTAGATTTATTAATTTTTCTGTGCTCAATGTGTAAATTGTATTATAATTC ATTGTGATTTATTTCACTTTTAATTTGCTGGTGTTTTAATAAATGGGGGTGTTACTGAATC TTTCTTCCCACTTCCATTTCTTTTGACCACCCCTTAACCCTCAACTGTGACGGTAGTAGT ATTATCATTTATACCAAAGTTTTGCATAGTCCCTGTTGACTTTGTAATGTTAACGGAGTC ATAAAAGCACTAGGCAAGAGAAAGATAGAAATTTGCTTTTAATCTTTTTGCCTTTTATTTT GCACATTATGCAAAAGGAAAAACATTAAAGAACACTTTTTTTTAAGTGAGTGAAAACATG GTAAAGACATACAGTGCTTTTATGCACATTGTTAAGCTAAATCAAGGTCATTTATAATCA TTTTCCTTTTTTATTTAAGATTTTAAGTAAACAAATTTTAGAATTTTCAGCATTTCAAAAAT GATTTTATTTTTCAAGTCTTAAATTCAATATTTTACACCTATGTTTTGAGGCTAAAAATAT GAAATTATATAATGTATGATACAGGGTTATCAAATATCTAATAATTTTTGAAATTAGCTCT TGTTTTTGGTTTTTTTGTTGTTGTTTTTACAGATTTCAGGTTACAAACTGCAAAGTTTATG CATAATTAAGTATGGTATGGTTGCCAGAAAAGCCTAAAATTACTACTTAGAAAATTTAAG ACTGTTTACCCCCATTGTCTTGTACTTGCGAGCTAACTTGTACTATTCTTGTGAAAGCAC TGTCATCTTTTAGTAGCAAATTTTGATAATGTTTCTCGTGGAAAAAAAAATCAGTATCTAT CTTTAGAACAATGTAATTATAATGTGGGAAGTGTGCATGAATGAGAGAGAGTGTGTGTG TATCTGTGTGTGTGTGCGCGTGTGTGTGTGTCTTTTAATAGTTTATGCCAGCAATCTTTG CTTGAATGTTTAACGATGCCTTCAGTGTGATGCTGGCCAATAGATGATTGCAGTTTAAA ATGTCATTATTGTGCAGGCTTGGATAACTAACATTCCATGATGTAGCTTGTTTCTGATGA GATGATTGTAGGTACATTTTTCTCATTATCCAATCATCTGTGGGATACTTAGTTTTCTAAT GTGCCATTATCTATTTTTATTCTGCAGTTATGTTCAAAATACAGTACATATTTTAAAATAG AATAAATTGTTAAACATAAAATTTTAAAAGTAGTAGATGTGCGTAAGAAAACTTTGTAAAA TAGTTATGAGTCCTACCCAGTAGCAACTTCTGGCATTCAAGCAGGATTCCACTATGTAA ATATCTGTAATGCATTTATAATAAGTTGTGTAGTTTGTCCTGCATCCATACTACACTATTT GCTAAAGTCTCAGTGCCATCTCCTAATGAGACTGACATTTTAAAAGTCTGTATGGAATAT CCTTGATAATTCAAGGAAATATCCCTCCTGCCTAAGTTCCAAACTGGGAAACATTCAAAT TATATAAATGACATTTCAGGACTTTAAGTATGAAGATAATGGGAATTTTATTGTTTTGCTT TTTAAAATGAGAGCATTTTTATTTGATAATTTTTTTTAAATTTTTAATTTTTAACTAATTTCA TTATTTTAAAGTAATCAGTTTTTCAAATCATGATTTTGATATCATTATTCTAAGGAGTTATC TCAAAGGCACAAAATATGAATTCTGCAAGAAGGCTATTTTTTATTGTAGTTTGAATGGGT TAGGAAAAGCCTCAATTTTTCACTCTTAAGTCCTTCAGTACATTTTTCTTTCATCTTATTA CTTATGCAAGTTAAGGTTCTTTGGTAACAGAATTCTTGCAACTGTAAAATAAAACTACAT AGATGTAAGAAGTCATGTAAACGGTTAACAAGCTTACCAAGGTTAGCAAAACTTTCATT GTAAATCAGTCTGTACTGAGCAAATAAAAATCATTATTAGTTGTATAAACACAAATTCCAT TTTGACTTTCAGGATGTCATACTACTTCTGTACCTAGCATTTTCAGTCCTTATATTTGCAA TGTTACACAAACTGTACTATTTTCTTTTATGTGCAGTTTGCATGAGTAAACCATCAGAGA ATAAATTCTATCTTTAAATTA. (SEQ ID NO:306; NM_001256090.2), or a nucleic acid sequence that hybridizes to a nucleic acid sequence consisting of SEQ ID NO:306 under stringent hybridization conditions

In some embodiments, activating transcription factor 3 (ATF3) comprises the amino acid sequence: MMLQHPGQVSASEVSASAIVPCLSPPGSLVFEDFANLTPFVKEELRFAIQNKHLCHRMSSA LESVTVSDRPLGVSITKAEVAPEEDERKKRRRERNKIAAAKCRNKKKEKTECLQKESEKLES VNAELKAQIEELKNEKQHLIYMLNLHRPTCIVRAQNGRTPEDERNLFIQQIKEGTLQS. (SEQ ID NO:307; NP_001025458.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:307)

In some embodiments, the nucleic acid sequence encoding ATF3 comprises the nucleic acid sequence: GGCGGAGGTGGGGTTAGCTTCAGTTGACCAACCATGCCTTGAGGATAAATTGGATGGG ATCAGATGGGAAGATGTGACAAGAAGAGAAATCCTCCTCTATATAGGATGCTCTGCTGT TTCCTAAGGATTTTCAGCACCTTGCCCCAAAATCAAAATGATGCTTCAACACCCAGGCC AGGTCTCTGCCTCGGAAGTGAGTGCTTCTGCCATCGTCCCCTGCCTGTCCCCTCCTGG GTCACTGGTGTTTGAGGATTTTGCTAACCTGACGCCCTTTGTCAAGGAAGAGCTGAGG TTTGCCATCCAGAACAAGCACCTCTGCCACCGGATGTCCTCTGCGCTGGAATCAGTCA CTGTCAGCGACAGACCCCTCGGGGTGTCCATCACAAAAGCCGAGGTAGCCCCTGAAG AAGATGAAAGGAAAAAGAGGCGACGAGAAAGAAATAAGATTGCAGCTGCAAAGTGCCG AAACAAGAAGAAGGAGAAGACGGAGTGCCTGCAGAAAGAGTCGGAGAAGCTGGAAAG TGTGAATGCTGAACTGAAGGCTCAGATTGAGGAGCTCAAGAACGAGAAGCAGCATTTG ATATACATGCTCAACCTTCATCGGCCCACGTGTATTGTCCGGGCTCAGAATGGGAGGA CTCCAGAAGATGAGAGAAACCTCTTTATCCAACAGATAAAAGAAGGAACATTGCAGAGC TAAGCAGTCGTGGTATGGGGGCGACTGGGGAGTCCTCATTGAATCCTCATTTTATACC CAAAACCCTGAAGCCATTGGAGAGCTGTCTTCCTGTGTACCTCTAGAATCCCAGCAGC AGAGAACCATCAAGGCGGGAGGGCCTGCAGTGATTCAGCAGGCCCTTCCCATTCTGC CCCAGAGTGGGTCTTGGACCAGGGCAAGTGCATCTTTGCCTCAACTCCAGGATTTAGG CCTTAACACACTGGCCATTCTTATGTTCCAGATGGCCCCCAGCTGGTGTCCTGCCCGC CTTTCATCTGGATTCTACAAAAAACCAGGATGCCCACCGTTAGGATTCAGGCAGCAGTG TCTGTACCTCGGGTGGGAGGGATGGGGCCATCTCCTTCACCGTGGCTACCATTGTCAC TCGTAGGGGATGTGGAGTGAGAACAGCATTTAGTGAAGTTGTGCAACGGCCAGGGTTG TGCTTTCTAGCAAATATGCTGTTATGTCCAGAAATTGTGTGTGCAAGAAAACTAGGCAAT GTACTCTTCCGATGTTTGTGTCACACAACACTGATGTGACTTTTATATGCTTTTTCTCAG ATCTGGTTTCTAAGAGTTTTGGGGGGCGGGGCTGTCACCACGTGCAGTATCTCAAGAT ATTCAGGTGGCCAGAAGAGCTTGTCAGCAAGAGGAGGACAGAATTCTCCCAGCGTTAA CACAAAATCCATGGGCAGTATGATGGCAGGTCCTCTGTTGCAAACTCAGTTCCAAAGTC ACAGGAAGAAAGCAGAAAGTTCAACTTCCAAAGGGTTAGGACTCTCCACTCAATGTCTT AGGTCAGGAGTTGTGTCTAGGCTGGAAGAGCCAAAGAATATTCCATTTTCCTTTCCTTG TGGTTGAAAACCACAGTCAGTGGAGAGATGTTTGGAAACCACAGTCAGTGGAGCCTGG GTGGTACCCAGGCTTTAGCATTATTGGATGTCAATAGCATTGTTTTTGTCATGTAGCTGT TTTAAGAAATCTGGCCCAGGGTGTTTGCAGCTGTGAGAAGTCACTCACACTGGCCACA AGGACGCTGGCTACTGTCTATTAAAATTCTGATGTTTCTGTGAAATTCTCAGAGTGTTTA ATTGTACTCAATGGTATCATTACAATTTTCTGTAAGAGAAAATATTACTTATTTATCCTAG TATTCCTAACCTGTCAGAATAATAAATATTGGAACCAAGACATGGTAAACAAAAAAAAAA AAAA. (SEQ ID NO:308; NM_001030287.3), or a nucleic acid sequence that hybridizes to a nucleic acid sequence consisting of SEQ ID NO:308 under stringent hybridization conditions

In some embodiments, ETS variant transcription factor 5 (ETV5, ERM) comprises the amino acid sequence: MDGFYDQQVPFMVPGKSRSEECRGRPVIDRKRKFLDTDLAHDSEELFQDLSQLQEAWLA EAQVPDDEQFVPDFQSDNLVLHAPPPTKIKRELHSPSSELSSCSHEQALGANYGEKCLYNY CAYDRKPPSGFKPLTPPTTPLSPTHQNPLFPPPQATLPTSGHAPAAGPVQGVGPAPAPHS LPEPGPQQQTFAVPRPPHQPLQMPKMMPENQYPSEQRFQRQLSEPCHPFPPQPGVPGD NRPSYHRQMSEPIVPAAPPPPQGFKQEYHDPLYEHGVPGMPGPPAHGFQSPMGIKQEPR DYCVDSEVPNCQSSYMRGGYFSSSHEGFSYEKDPRLYFDDTCVVPERLEGKVKQEPTMY REGPPYQRRGSLQLWQFLVTLLDDPANAHFIAWTGRGMEFKLIEPEEVARRWGIQKNRPA MNYDKLSRSLRYYYEKGIMQKVAGERYVYKFVCDPDALFSMAFPDNQRPFLKAESECHLS EEDTLPLTHFEDSPAYLLDMDRCSSLPYAEGFAY. (SEQ ID NO:309; NP_004445.1), or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:309)

In some embodiments, the nucleic acid sequence encoding ETV5 comprises the nucleic acid sequence: AGAGTCCAGCCGCTGGTGCGCGGAGCGGTTCACCGTCTTCGGAGCGGTTCGGCCCAG CCTTTCGCCCAGGCGCCCAGGCCCGCTGCGCGCGTGCGTGAGCGCGCCTGCGCCGC CGGGGCCGCTGCAAGGGGAGGAGAGAGGCCGCCTCAGGAGGATCCCTTTTCCCCCA GAAATTACTCAATGCTGAAACCTCTCAAAGTGGTATTAGAGACGCTGAAAGCACCATGG ACGGGTTTTATGATCAGCAAGTCCCTTTTATGGTCCCAGGGAAATCTCGATCTGAGGAA TGCAGAGGGCGGCCTGTGATTGACAGAAAGAGGAAGTTTTTGGACACAGATCTGGCTC ACGATTCTGAAGAGCTATTTCAGGATCTCAGTCAACTTCAAGAGGCTTGGTTAGCTGAA GCACAAGTTCCTGATGATGAACAGTTTGTCCCAGATTTTCAGTCTGATAACCTGGTGCT TCATGCCCCACCTCCAACCAAGATCAAACGGGAGCTGCACAGCCCCTCCTCTGAGCTG TCGTCTTGTAGCCATGAGCAGGCTCTTGGTGCTAACTATGGAGAAAAGTGCCTCTACAA CTATTGTGCCTATGATAGGAAGCCTCCCTCTGGGTTCAAGCCATTAACCCCTCCTACAA CCCCCCTCTCACCCACCCATCAGAATCCCCTATTTCCCCCACCTCAGGCAACTCTGCC CACCTCAGGGCATGCCCCTGCAGCTGGCCCAGTTCAAGGTGTGGGCCCCGCCCCCGC CCCCCATTCGCTTCCAGAGCCTGGACCACAGCAGCAAACATTTGCGGTCCCCCGACCA CCACATCAGCCCCTGCAGATGCCAAAGATGATGCCTGAAAACCAGTATCCATCAGAAC AGAGATTTCAGAGACAACTGTCTGAACCCTGCCACCCCTTCCCTCCTCAGCCAGGAGT TCCTGGAGATAATCGCCCCAGTTACCATCGGCAAATGTCAGAACCTATTGTCCCTGCAG CTCCCCCGCCCCCTCAGGGATTCAAACAAGAATACCATGACCCACTCTATGAACATGG GGTCCCGGGCATGCCAGGGCCCCCAGCACACGGGTTCCAGTCACCAATGGGAATCAA GCAGGAGCCTCGGGATTACTGCGTCGATTCAGAAGTGCCTAACTGCCAGTCATCCTAC ATGAGAGGGGGTTATTTCTCCAGCAGCCATGAAGGTTTTTCATATGAAAAAGATCCCCG ATTATACTTTGACGACACTTGTGTTGTGCCTGAGAGACTGGAAGGCAAAGTCAAACAGG AGCCTACCATGTATCGAGAGGGGCCCCCTTACCAGAGGCGAGGTTCCCTTCAGCTGTG GCAGTTCCTGGTCACCCTTCTTGATGACCCAGCCAATGCCCACTTCATTGCCTGGACA GGTCGAGGCATGGAGTTCAAGCTGATAGAACCGGAAGAGGTTGCTCGGCGCTGGGGC ATCCAGAAGAACCGGCCAGCCATGAACTATGACAAGCTGAGCCGCTCTCTCCGCTATT ACTATGAAAAGGGCATCATGCAGAAGGTGGCTGGAGAGCGATACGTCTACAAATTTGT CTGTGACCCAGATGCCCTCTTCTCCATGGCTTTCCCGGATAACCAGCGTCCGTTCCTG AAGGCAGAGTCCGAGTGCCACCTCAGCGAGGAGGACACCCTGCCGCTGACCCACTTT GAAGACAGCCCCGCTTACCTCCTGGACATGGACCGCTGCAGCAGCCTCCCCTATGCC GAAGGCTTTGCTTACTAAGTTTCTGAGTGGCGGAGTGGCCAAACCCTAGAGCTAGCAG TTCCCATTCAGGCAAACAAGGGCAGTGGTTTTGTTTGTGTTTTTGGTTGTTCCTAAAGCT TGCCCTTTGAGTATTATCTGGAGAACCCAAGCTGTCTCTGGATTGGCACCCTTAAAGAC AGATACATTGGCTGGGGAGTGGGAACAGGGAGGGGCAGAAAACCACCAAAAGGCCAG TGCCTCAACTCTTGATTCTGATGAGGTTTCTGGGAAGAGATCAAAATGGAGTCTCCTTA CCATGGACAATACATGCAAAGCAATATCTTGTTCAGGTTAGTACCCGCAAAACGGGACA TAGTATGTGACAATCTGCATCGATCATGGACTACTAAATGCCTTTACATAGAAGGGCTC TGATTTGCACAATTTGTTGAAAAATCACAAACCCATAGAAAAGTAAGTAGGCTAAGTTGG GGAGGCTCAAACCATTAAGGGTTAAAAATACATCTTAAACATTGGAAAGCTCTTCTAGC TGAATCTGAAATATTACCCCTTGTCTAGAAAAAGGGGGGCAGTCAGAACAGCTGTTCCC CACTCCGTGGTTCTCAAAATCATAAACCATGGCTACTCTTGGGAACCACCCGGCCATGT GGTCGCCAAGTAGAGCAAGCCCCCTTTCTCTTCCCAATCACGTGGCTGAGTGTGGATG ACTTTTATTTTAGGAGAAGGGCGATTAACACTTTTGACAGTATTTTGTTTTGCCCTGATT TGGGGGATTGTTTTGTTTTGGTGGTTGTTTTGGAAAAACAGTTTATAAACTGATTTTTGT AGTTTTGGTATTTAAAGCAAAAAAACGAAAAACAAAAAACAAAAACAAACCTTTTGGTAA CTGTGCACTGTGTCCTTTAGCCAGGGCCGTGCCAACTTATGAAGACACTGCAGCTTGA GAGGGGCTTTGCTGAGGCTTCCCCTTGGCCATGTGAAAGCCCGCCTTGTTGCCTGCTT TGTGCTTTCTGCACCAGACAACCTGATGGAACATTTGCACCTGAGTTGTACATTTTTGA AGTGTGCAGGGCAGCCTGGACACAAGCTTAGATTCTCTATGTATAGTTCCCCGTGTTCA CTAACATGCCCTCTCTGGAAAGCATATGTATATAACATGTGTCATGTCCTTTGGAAACCT GGTCACCTGGTGAAAACCCTTGGGATTCTTCCCTGGGCATGACTGATGACAATTTCCAT TTCATCAGTTTGTTTTGTTTTCCTTTTTCTTTAAATCTTGGACTTTAAACCCTACCTGTGT GATTCAGTAGGGTTTGAGACTTACGTGTGATACTGACAGGTAAGCAACAGTGCTAGCAT TCTAGATTCCTGCCTTTTTTTAAAAAGAAATTATTCTCATTGCTGTATTATATTGGAAAAG TTTTAAACAACCAAGCTAAAGCTATGTGAAAGTTGAGCTCAAAGTAGAGGAAAAGTTAC TGGTGGTACCTTGCTGCCTGCTCTGCTGGTAGAATTCTGTGCTCCCCGTGACACTTAGT ACATTAAGAATGACTACACTGTTCCTCGTATGTGAAGGAGGCAGTGCTGACTCCGTGA GTGTGAGACACGTGCTTTGAACTGCTTTTCTATTCATGGAGCACTCCATAGTCTCAAAC TGTCCCCCTTATGACCAACAGCACATTTGTGAAGAGGTTCGCAGGGATAAGGGGTGCA CTTTATAGCTATGGAAACATGAGATTCTCCTCTATTGGAAGCTAATTAGCCCACAAAGGT GGTAAACCTGTAGATTGGGCCTTAATTAGCATTGTACTCTAATCAAAGGACTCTTTCTAA ACCATATTTATAGCTTTCTTAACCTACACATAGTCTATACATAGATGCATATTTTACCCCC AGCTGGCTAGAGATTTATTTGTTGTAAATGCTGTATAGATTTGGTTTTCCTTTCTTTACTT ACCCTGGTTTGGATTTTTTTTTTTTTTCTTTTGAATGGATTTATGCTGTCTTAGCAATATG ACAATAATCCTCTGTAGCTTGAGCTACCCCTCCCCTGCTGTAACTTACGTGACCTGTGC TGTCACTGGGCATAGGACAGCGGCATCACGGTTGCATTCCCATTGGACTCATGCACCT CCCGGATGGTTTTTGTTTTTTTCGGGGGTTCTTTGGGGTTTGTTTGTTTGCTTCTTTTCC AGAGTGTGGAAAGTCTACAGTGCAGAAAGGCTTGAACCTGCCAGCTGATTTGAAATACT TTCCCCTGCGCAGGGCCGTATGCATCCTGCCAAGCTGCGTTATATTCTGTACTGTGTAC AATAAAGAAGTTTGCTTTTCGTTTACCAAGCA. (SEQ ID NO:310; NM_004454.3), or a nucleic acid sequence that hybridizes to a nucleic acid sequence consisting of SEQ ID NO:310 under stringent hybridization conditions. In order to express a polypeptide or functional nucleic acid, the nucleotide coding sequence may be inserted into appropriate expression vector. Therefore, also disclosed is a non-viral vector comprising a polynucleotide comprising one or more nucleic acid sequences encoding the disclosed transcription factors, wherein the one or more nucleic acid sequences are operably linked to an expression control sequence. In some embodiments, the nucleic acid sequences are operably linked to a single expression control sequence. In other embodiments, the nucleic acid sequences are operably linked to two or more separate expression control sequences. In some embodiments, the non-viral vector comprises a plasmid selected from the group pIRES-hrGFP-21, pAd-IRES-GFP, pCMV6-AC-GFP, and pCDNA3.0.

Methods to construct expression vectors containing genetic sequences and appropriate transcriptional and translational control elements are well known in the art. These methods include in vitro recombinant DNA techniques, synthetic techniques, and in vivo genetic recombination. Such techniques are described in Sambrook et al., Molecular Cloning, A Laboratory Manual (Cold Spring Harbor Press, Plainview, N.Y., 1989), and Ausubel et al., Current Protocols in Molecular Biology (John Wiley & Sons, New York, N.Y., 1989).

Expression vectors generally contain regulatory sequences necessary elements for the translation and/or transcription of the inserted coding sequence. For example, the coding sequence is preferably operably linked to a promoter and/or enhancer to help control the expression of the desired gene product.

The “control elements” or “regulatory sequences” are those non-translated regions of the vector—enhancers, promoters, 5′ and 3′ untranslated regions-which interact with host cellular proteins to carry out transcription and translation. Such elements may vary in their strength and specificity.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression.

An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

Promoters used in biotechnology are of different types according to the intended type of control of gene expression. They can be generally divided into constitutive promoters, tissue-specific or development-stage-specific promoters, inducible promoters, and synthetic promoters.

Constitutive promoters direct expression in virtually all tissues and are largely, if not entirely, independent of environmental and developmental factors. As their expression is normally not conditioned by endogenous factors, constitutive promoters are usually active across species and even across kingdoms. Examples of constitutive promoters include CMV, EF1a, SV40, PGK1, Ubc, Human beta actin, and CAG.

Tissue-specific or development-stage-specific promoters direct the expression of a gene in specific tissue(s) or at certain stages of development. For plants, promoter elements that are expressed or affect the expression of genes in the vascular system, photosynthetic tissues, tubers, roots and other vegetative organs, or seeds and other reproductive organs can be found in heterologous systems (e.g. distantly related species or even other kingdoms) but the most specificity is generally achieved with homologous promoters (i.e. from the same species, genus or family). This is probably because the coordinate expression of transcription factors is necessary for regulation of the promoter's activity.

The performance of inducible promoters is not conditioned to endogenous factors but to environmental conditions and external stimuli that can be artificially controlled. Within this group, there are promoters modulated by abiotic factors such as light, oxygen levels, heat, cold and wounding. Since some of these factors are difficult to control outside an experimental setting, promoters that respond to chemical compounds, not found naturally in the organism of interest, are of particular interest. Along those lines, promoters that respond to antibiotics, copper, alcohol, steroids, and herbicides, among other compounds, have been adapted and refined to allow the induction of gene activity at will and independently of other biotic or abiotic factors.

The two most commonly used inducible expression systems for research of eukaryote cell biology are named Tet-Off and Tet-On. The Tet-Off system makes use of the tetracycline transactivator (tTA) protein, which is created by fusing one protein, TetR (tetracycline repressor), found in Escherichia coli bacteria, with the activation domain of another protein, VP16, found in the Herpes Simplex Virus. The resulting tTA protein is able to bind to DNA at specific TetO operator sequences. In most Tet-Off systems, several repeats of such TetO sequences are placed upstream of a minimal promoter such as the CMV promoter. The entirety of several TetO sequences with a minimal promoter is called a tetracycline response element (TRE), because it responds to binding of the tetracycline transactivator protein tTA by increased expression of the gene or genes downstream of its promoter. In a Tet-Off system, expression of TRE-controlled genes can be repressed by tetracycline and its derivatives. They bind tTA and render it incapable of binding to TRE sequences, thereby preventing transactivation of TRE-controlled genes. A Tet-On system works similarly, but in the opposite fashion. While in a Tet-Off system, tTA is capable of binding the operator only if not bound to tetracycline or one of its derivatives, such as doxycycline, in a Tet-On system, the rtTA protein is capable of binding the operator only if bound by a tetracycline. Thus the introduction of doxycycline to the system initiates the transcription of the genetic product. The Tet-On system is sometimes preferred over Tet-Off for its faster responsiveness.

In some embodiments, the nucleic acid sequences encoding the disclosed transcription factors are operably linked to the same expression control sequence. Alternatively, internal ribosome entry sites (IRES) elements can be used to create multigene, or polycistronic, messages. IRES elements are able to bypass the ribosome scanning model of 5′ methylated Cap dependent translation and begin translation at internal sites. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Disclosed are non-viral vectors containing one or more polynucleotides disclosed herein operably linked to an expression control sequence. Examples of such non-viral vectors include the oligonucleotide alone or in combination with a suitable protein, polysaccharide or lipid formulation. Non-viral methods present certain advantages over viral methods, with simple large scale production and low host immunogenicity being just two. Previously, low levels of transfection and expression of the gene held non-viral methods at a disadvantage; however, recent advances in vector technology have yielded molecules and techniques with transfection efficiencies similar to those of viruses.

Examples of suitable non-viral vectors include, but are not limited to pIRES-hrGFP-2a, pAd-IRES-GFP, and pCDNA3.0.

The compositions disclosed can be used therapeutically in combination with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable..

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The herein disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

### Methods

Also disclosed are methods of reprogramming diseased musculoskeletal cells that involve delivering intracellularly into the somatic cells a polynucleotide comprising one or more nucleic acid sequences encoding the disclosed transcription factors. In some embodiments, the nucleic acid sequences are present in non-viral vectors. In some embodiments, the nucleic acid sequences are operably linked to an expression control sequence. In other embodiments the nucleic acids are operably linked to two or more expression control sequences.

A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion.

In some embodiments, after transfecting target cells with the disclosed polynucleotides, the cells can then pack the transfected genes (e.g. cDNA) into EVs, which can then induce endothelium in other somatic cells. Therefore, also disclosed is a method of reprogramming diseased musculoskeletal cells that involves exposing the cells with an extracellular vesicle produced from a cell containing or expressing the disclosed transcription factors.

Therefore, disclosed are methods of reprogramming diseased musculoskeletal cells that involve exposing the cells to extracellular vesicles (EVs) isolated from cells expressing or containing exogenous polynucleotides comprising one or more nucleic acid sequences encoding the disclosed transcription factors. For example, in some embodiments, the donor cells are transfected with the one or more disclosed polynucleotides and cultured in vitro. EVs secreted by the donor cells can then collected from the culture medium. These EVs can then be administered to the diseased musculoskeletal to reprogram them into healthy cells. In some embodiments, the donor cells can be any viable musculoskeletal cells or skin cells, including (but not limited to) NP, AF, CEPs, Articular Chondrocytes, tenocytes, and osteoblasts.

Exosomes and microvesicles are EVs that differ based on their process of biogenesis and biophysical properties, including size and surface protein markers. Exosomes are homogenous small particles ranging from 40 to 150 nm in size and they are normally derived from the endocytic recycling pathway. In endocytosis, endocytic vesicles form at the plasma membrane and fuse to form early endosomes. These mature and become late endosomes where intraluminal vesicles bud off into an intra-vesicular lumen. Instead of fusing with the lysosome, these multivesicular bodies directly fuse with the plasma membrane and release exosomes into the extracellular space. Exosome biogenesis, protein cargo sorting, and release involve the endosomal sorting complex required for transport (ESCRT complex) and other associated proteins such as Alix and Tsg101. In contrast, microvesicles, are produced directly through the outward budding and fission of membrane vesicles from the plasma membrane, and hence, their surface markers are largely dependent on the composition of the membrane of origin. Further, they tend to constitute a larger and more heterogeneous population of extracellular vesicles, ranging from 150 to 1000 nm in diameter. However, both types of vesicles have been shown to deliver functional mRNA, miRNA and proteins to recipient cells.

In some embodiments, the polynucleotides are delivered to the somatic cells, or the donor cells for EVs, intracellularly via a gene gun, a microparticle or nanoparticle suitable for such delivery, transfection by electroporation, three-dimensional nanochannel electroporation, a tissue nanotransfection device, a liposome suitable for such delivery, or a deep-topical tissue nanoelectroinjection device. In some embodiments, a viral vector can be used. However, in other embodiments, the polynucleotides are not delivered virally.

Electroporation is a technique in which an electrical field is applied to cells in order to increase permeability of the cell membrane, allowing cargo (e.g., reprogramming factors) to be introduced into cells. Electroporation is a common technique for introducing foreign DNA into cells.

Tissue nanotransfection allows for direct cytosolic delivery of cargo (e.g., reprogramming factors) into cells by applying a highly intense and focused electric field through arrayed nanochannels, which benignly nanoporates the juxtaposing tissue cell members, and electrophoretically drives cargo into the cells.

In one embodiment, the disclosed compositions are administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of the disclosed compositions administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

In some embodiments, the disclosed compositions and methods are used to create a vasculature that can serve as a scaffolding structure. This scaffolding structure can then be used, for example, to aid in the repair of nerve tissue. Applications of this include peripheral nerve injuries, and pathological/injurious insults to the central nervous system such as traumatic brain injury or stroke. In some embodiments, the created vasculature can be used to nourish composite tissue transplants, or any tissue graft.

In some embodiments, the disclosed compositions and methods are used to convert “unwanted” tissue (e.g., fat, scar tissue) into vasculature. Such newly formed vasculature is expected to “resorb” under non-ischemic conditions.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: FOXF1 Transfection for Trans-Differentiation of Diseased Intervertebral Disc Cells into a Healthy Phenotype

Low back pain affects 70-85% of the world's population and is the leading cause of disability worldwide with over $100 billion in medical expenses in the U.S.A Alone. Intervertebral Disc degeneration is a main contributor of low back pain but current therapies do not target the underlying disease.

Such treatments include surgical interventions and medication which can result in non-unions, nerve injuries, etc. Some proposed treatments include tissue engineering, cell therapy, and even injectable hydrogel constructs. However, those treatments are under desired by clinicians and lack mechanical integrity of the IVD.

A native healthy IVD is gelatinous in the middle (Nucleus Pulposus) with surrounding fibers (Annulus Fibrosus). It's the largest avascular organ in the body. However, with aging, increased mechanical loads, and unknown disease pathologies, the disc degenerates. This degeneration has shown to cause pain due to pressure on the spinal cord along with unwarranted neurovascular invasion.

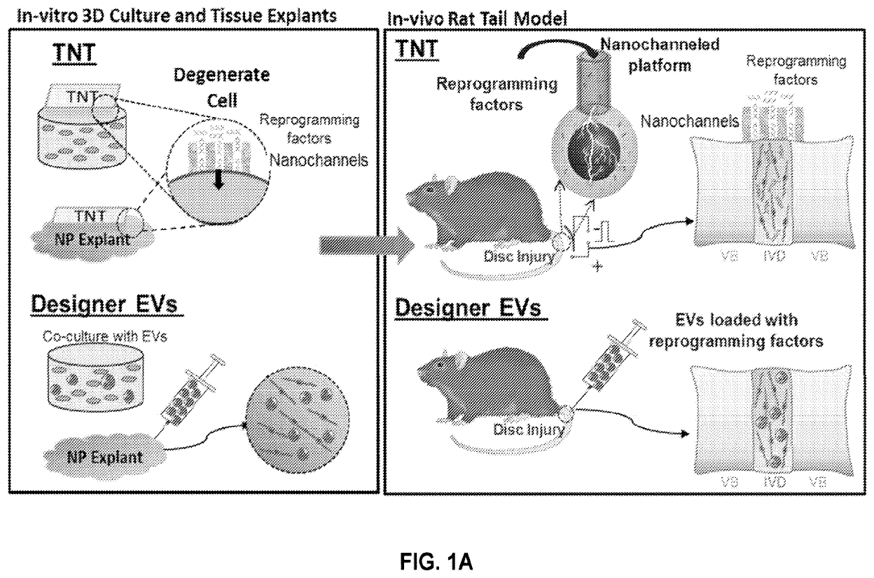

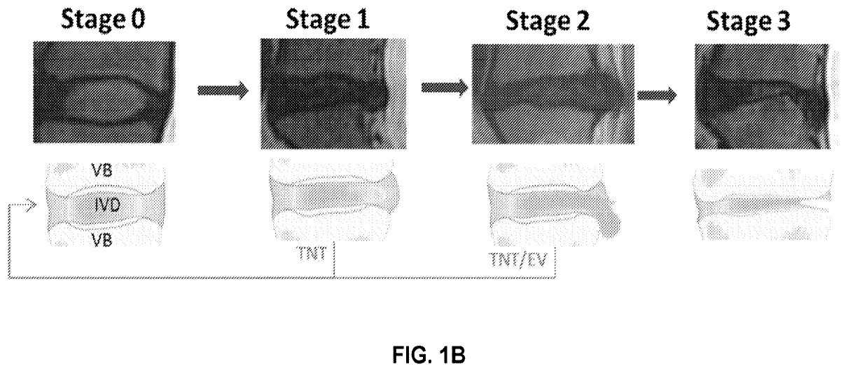

The overarching goal of this technology is to use the combination of transcription factor and TNT/Evs to revert diseased intervertebral disc cells to a healthy phenotype (FIG. 1).

### Methods

Transcription factor plasmid expansion via transformation into DH5a E. coli cells, selectivity via ampicillin resistance and plasmid DNA isolated

NP cells isolated from IVD tissue of human patients (n=5) undergoing spinal surgery and cadaveric tissue and expanded in monolayer until 80% confluent.

FOX Family transcription factor or non-transcription factor containing vector (SHAM) transfected in NP cells via bulk electroporation Neon™ Transfection System MPK5000 (V=1425 Volts, t=30 msec, 1 Pulse). Cells expanded and seeded in 2% Agarose Gels (Ø=8 mm, H=4 mm).

Gels taken down for analysis at Day 0, Week 2, and week 4 and analyzed for: cell viability (Live/Dead Assay Calcein/Ethidium), gene expression (qPCR) and Glycosaminoglycan (GAG) content (Dimethyl methylene Blue Assay (DMMB) normalized to DNA (Hoechst Assay).

Mann Whitney statistical tests used to evaluate significance at α=0.05.

Thompson Grading is a standard grade for disc degeneration where 1=healthy and 5=degenerate. Autopsy samples are graded as they do not come directly from diseased tissue. Surgical samples are diseased tissue removed from patients during routine spinal surgery. Table 2 shows human surgical and autopsy NP cells expanded 2 weeks.

TABLE 2
Human autopsy and surgical specimen demographic with level
Thompson grade for Autopsy and level for Surgical

| Autopsy | | | | | Surgical | | | |
|---|---|---|---|---|---|---|---|---|
| | | Age | | | | | Age | |
| ID | Sex | (years) | Level | Grade | ID | Sex | (years) | Level |
| Hu-4 | Female | 49 | L2-L3 | 2.5 | Hs-2 | Male | 26 | L5-S1 |
| Hu-6 | Male | 45 | L2-L3 | 3 | Hs-11 | Male | 28 | L5-S1 |
| Hu-7 | Female | 56 | L2-L3 | 2.5 | Hs-29 | Female | 70 | L5-S1 |
| Hu-9 | Female | 58 | L4-L5 | 2.5 | Hs-34 | Female | 19 | L5-S1 |
| Hu-16 | Female | 19 | L1-L2 | 1.5 | Hs-39 | Male | 60 | L5-S1 |

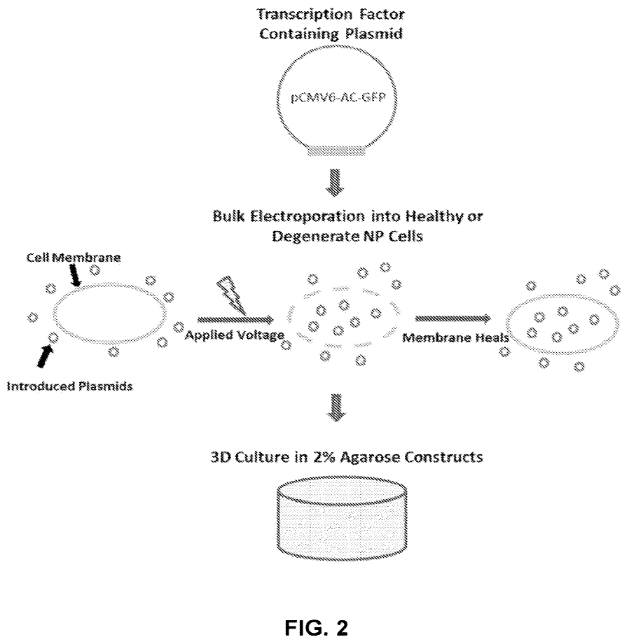

FIG. 2 is a schematic of DNA bulk electroporation into NP cells then seeded in Agarose Gel.

### Results

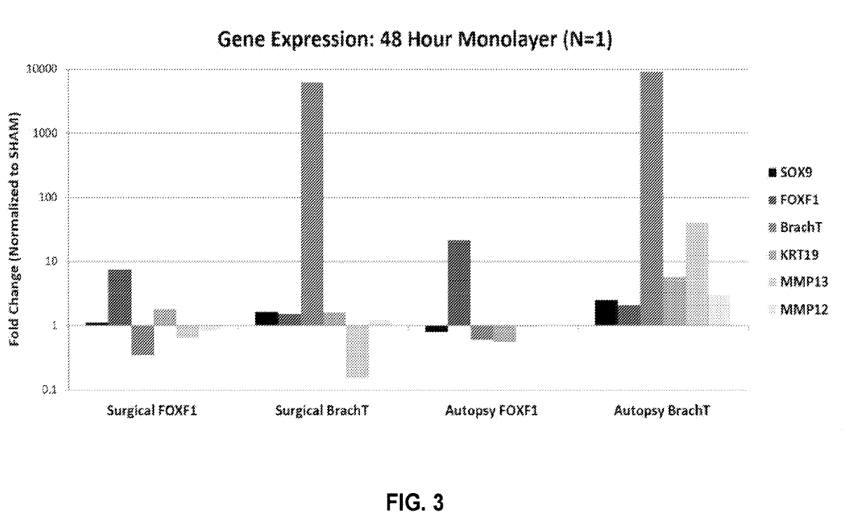

FIG. 3 is a graph showing qPCR Gene expression data validating that the transcription factor was successfully transmitted. X-axis=type of tissue and transcription factor. Colors indicate the gene being tested for.

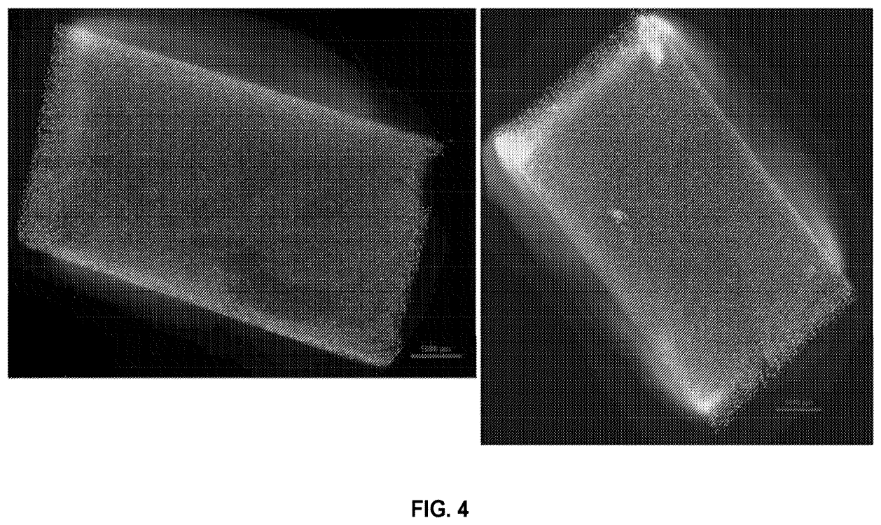

FIG. 4 contains representative viability images (4× Stitched) of Gels at day 0 and 4 Weeks. (Green=Live, Red=Dead).

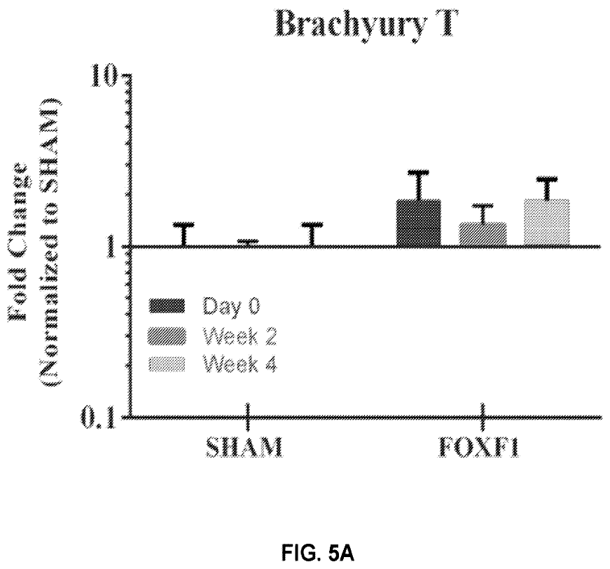

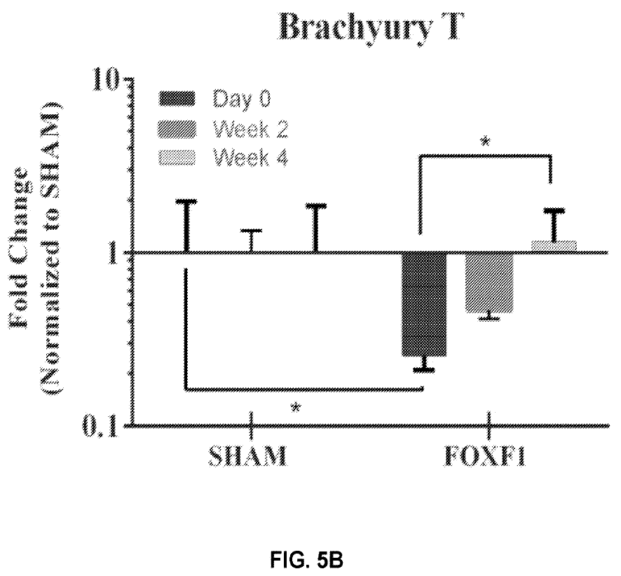

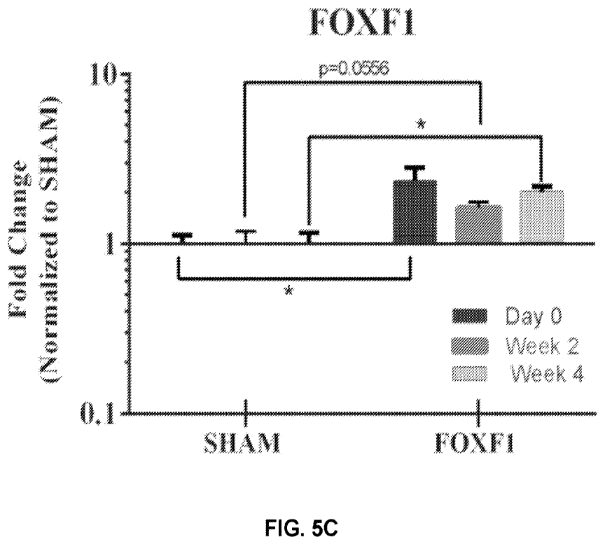

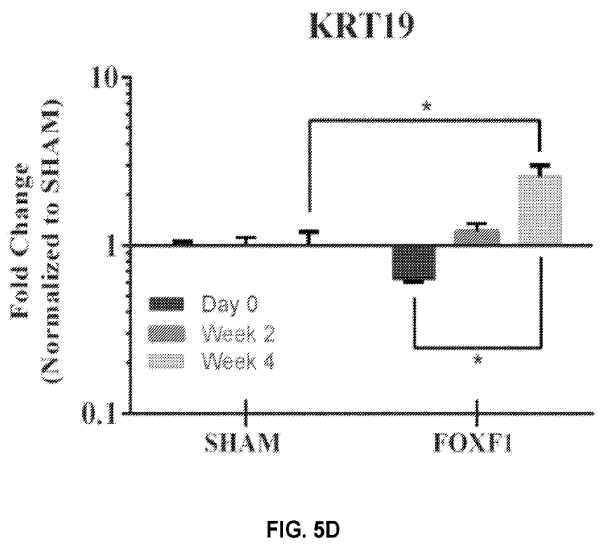

FIGS. 5A and 5B are graphs showing Brachyury T expression in autopsy (FIG. 5A) and surgical (FIG. 5B) nucleus pulposus cells after sham or FOXF1 treatment. FIGS. 5C and 5D are graphs showing FOXF1 (FIG. 5C) and KRT19 (FIG. 5D) expression in healthy nucleus pulposus cells after sham or FOXF1 treatment.

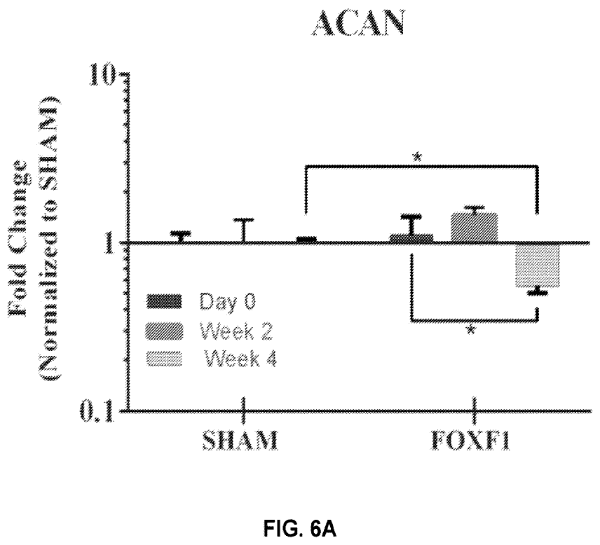

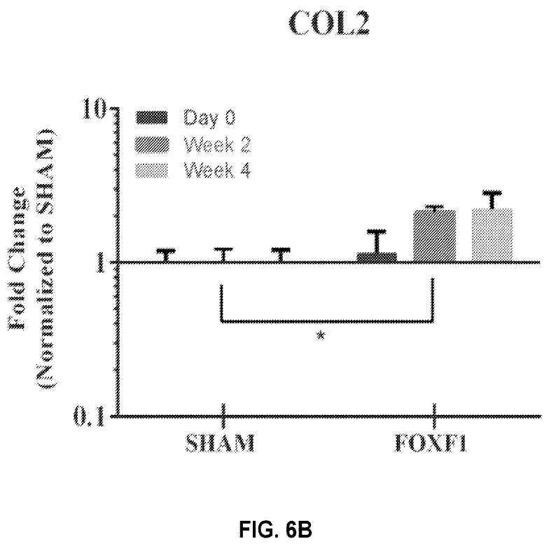

FIGS. 6A and 6B are graphs showing ACAN (FIG. 6A) and COL2 (FIG. 6B) expression in healthy nucleus pulposus cells after sham or FOXF1 treatment.

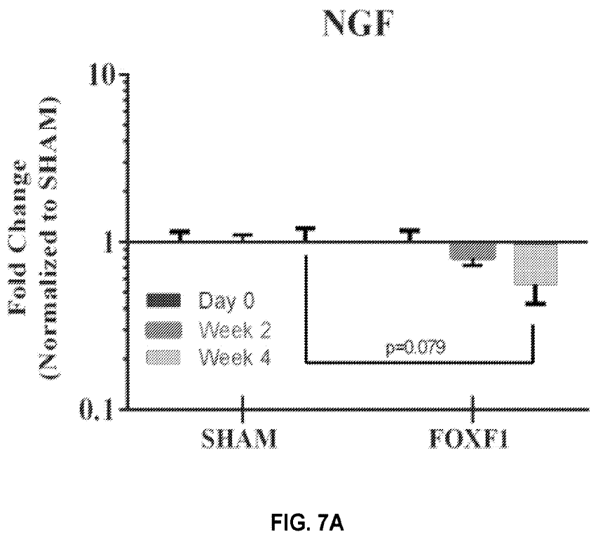

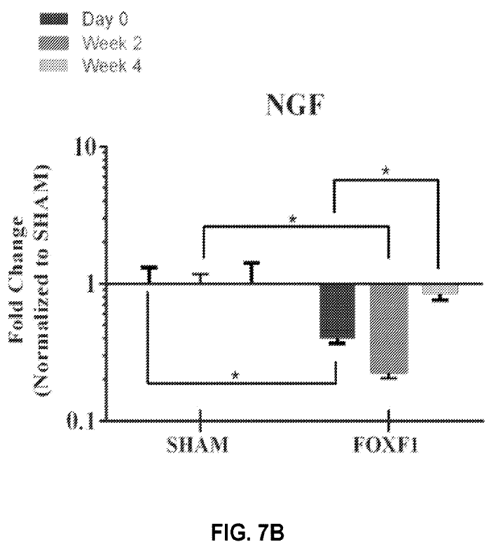

FIGS. 7A and 7B are graphs showing NGF expression in autopsy (FIG. 7A) and surgical (FIG. 7B) nucleus pulposus cells after sham or FOXF1 treatment.

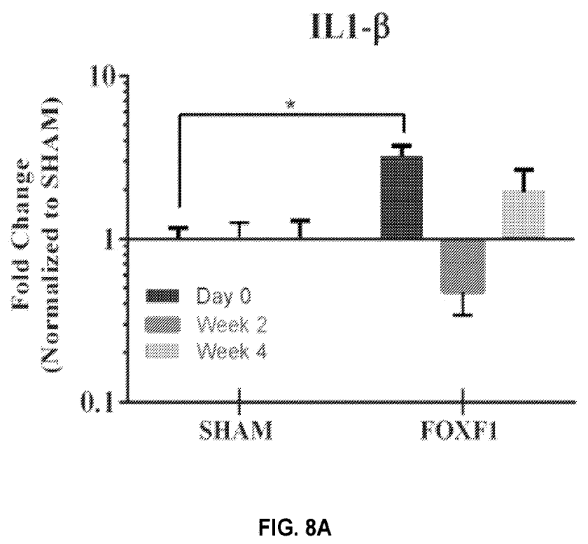

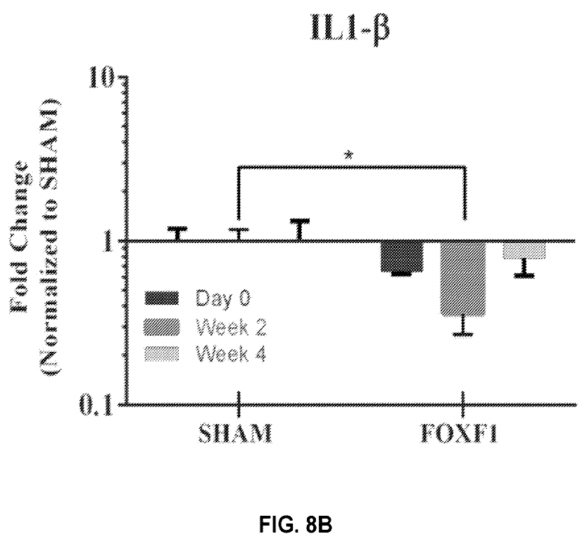

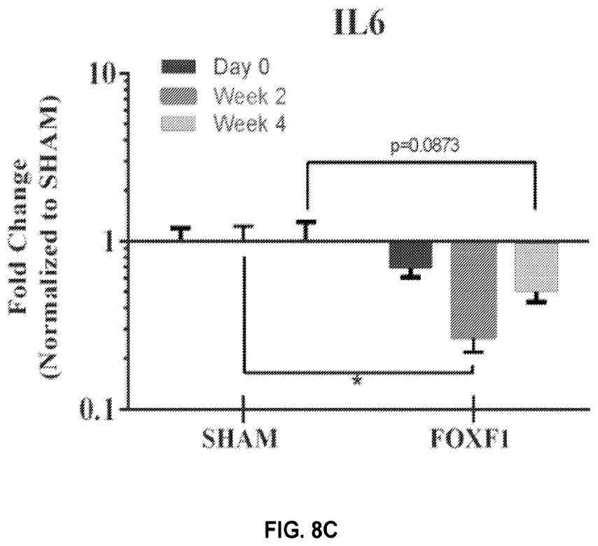

FIGS. 8A and 8B are graphs showing IL1-3 expression in autopsy (FIG. 8A) and surgical (FIG. 8B) nucleus pulposus cells after sham or FOXF1 treatment. FIG. 8C is a graph showing IL6 expression in nucleus pulposus cells after sham or FOXF1 treatment.

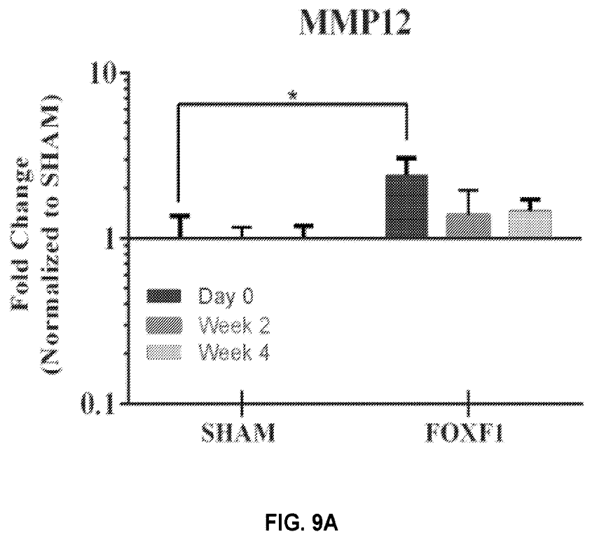

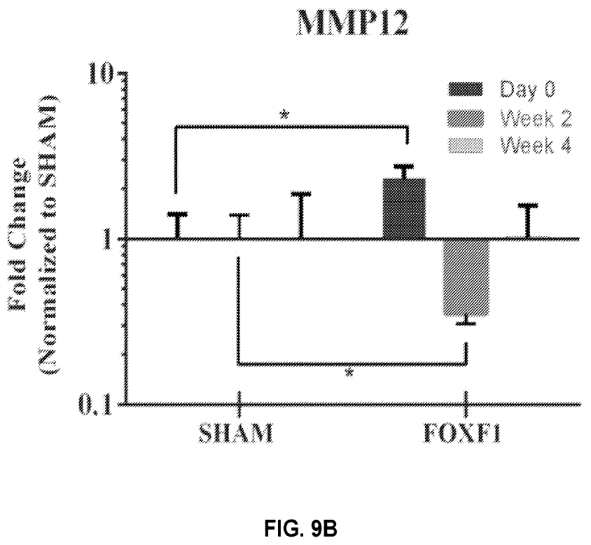

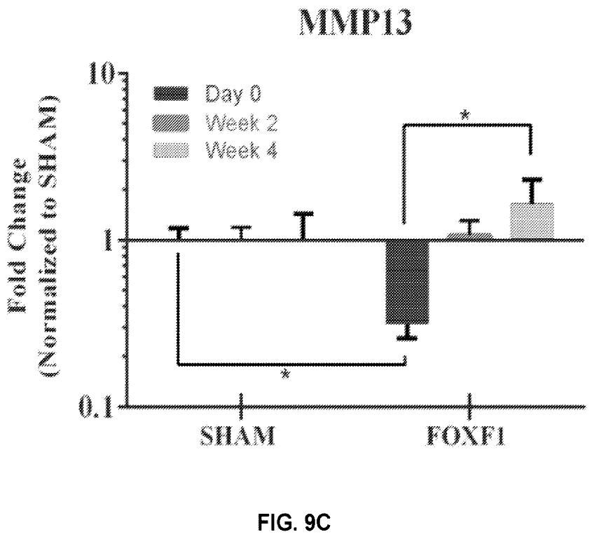

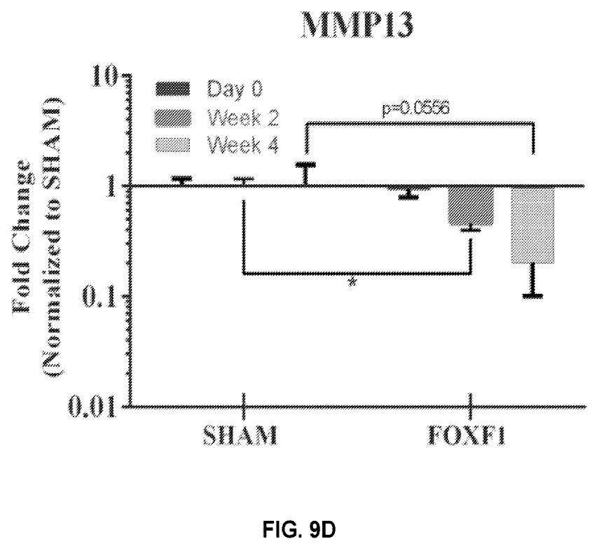

FIGS. 9A and 9B are graphs showing MMP12 expression in autopsy (FIG. 9A) and surgical (FIG. 9B) nucleus pulposus cells after sham or FOXF1 treatment. FIGS. 9C and 9D are graphs showing MMP13 expression in autopsy (FIG. 9C) and surgical (FIG. 9D) nucleus pulposus cells after sham or FOXF1 treatment.

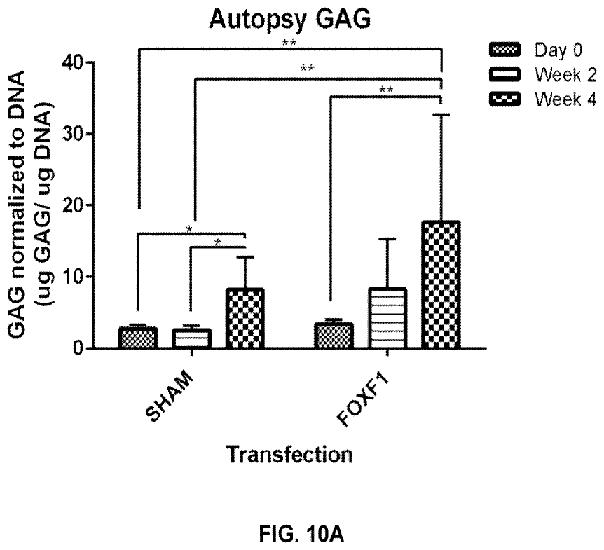

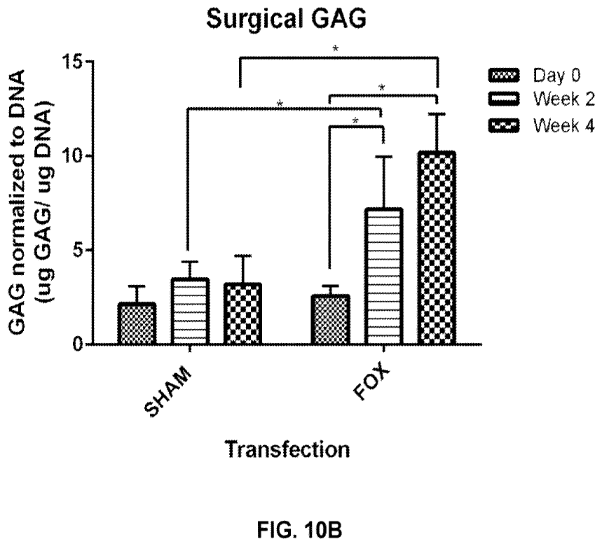

FIGS. 10A and 10B are bar graphs showing GAG content in autopsy (FIG. 10A) and surgical (FIG. 10B) nucleus pulposus cells after sham or FOXF1 treatment.

### Conclusion

This study demonstrates: (i) the ability to transfect degenerate cells using bulk electroporation, (ii) transfected cell maintained viability over 4 weeks of culture in 3D constructs, (iii) introduction of FOX family gene into the cytosolic environment of the cell induces proteoglycan (GAG) production critical for IVD function and (iv) inhibition of inflammatory and neuron growth factor.

### Example 2: Non-Viral Transfection of Human Intervertebral Disc Cells with Developmental Factors Induces Reprogramming to a Healthy Anti-Catabolic/Inflammatory Phenotype with Enhanced Extracellular Matrix Accumulation

Low back pain (LBP) is the leading cause of disability worldwide with an associated socioeconomic burden of over $100 billion annually in the U.S alone [Katz, J. et al, 2006]. Intervertebral disc (IVD) degeneration is a major contributor to LBP and is characterized by decreases in cellularity and proteoglycan synthesis, upregulation of matrix degrading enzymes (MMPs), and increases in pro-inflammatory factors with neurovascular invasion[Rodriques-Pinto R. et al, 2014; Freemont A. J. et al, 2009]. Current treatment strategies are highly invasive and fail to target the underlying pathology or promote tissue repair. Pro-anabolic approaches have been proposed which includes gene therapy through viral infection, but this has raised safety concerns due to mutagenesis and unwarranted immune responses. To avoid such safety risks, electroporation of plasmids carrying DNA for transcription factors can be introduced into endogenous cells without alteration of native DNA to stimulate IVD repair. The transcription factor, Brachyury (BrachT), is expressed in the developing notochord and is associated with maintaining a healthy immature nucleus pulposus (NP) phenotype [Vujovic, S. et al, 2006; Tang, R. et al, 2018]. As disclosed herein, delivery of BrachT into degenerate human IVD cells can reprogram diseased NP cells into healthy cells with increased proteoglycan and decreased inflammatory, catabolic and pain associated factors which are critical for maintaining the structure and function of the healthy IVD. Thus, the overall objective of this study was to examine the effects of BrachT transfection on human NP cell phenotype and function.

### Methods

BrachT transcription factor plasmids (OriGene Tech, Cat: SC303281) were expanded via transformation into DH5a E. coli cells with ampicillin resistance and plasmid DNA isolated for downstream electroporation. Human NP cells were isolated from non-degenerate (ND) cadaveric IVDs from autopsy (n=5, 19-58 y.o) or from the painful-degenerate (PD) IVD tissue of human patients with back pain (n=5, 19-70 y.o, IRB: 2015H0385) undergoing microdiscectomy (2 mg/mL Pronase-1 hour, 2 mg/mL Collagenase II-4 hours). NP cells were expanded in monolayer (p2) until 80% confluent before bulk electroporation with empty plasmids (SHAM) or BrachT plasmids via Neon™ Transfection System MPK5000 (V=1425 Volts, t=30 msec, 1 Pulse). Successful transfection was verified with RT-qPCR at 48 hours. Transfected cells were then expanded in disc cell media (High glucose DMEM, 10% FBS, 1% P/S, 50 μg/ml ascorbic acid fresh) and seeded in 2% 3D agarose gel constructs at 20 E6 cells/mL. Dependent variables were examined at day 0, week 2 and week 4 for cell viability (Calcein/Ethidium staining), extracellular matrix, phenotypic marker and inflammatory/catabolic gene expression (RT-qPCR) and proteoglycan/GAG content (Dimethylmethylene Blue Assay with DNA/Hoechst normalization). Non-parametric statistical tests were used (Mann-Whitney Tests, α=0.05).

### Results

Cell viability remained high for all groups and BrachT gene expression was maintained over 4 weeks in both ND and PD cells with a decline in PD cells at week 4 only (fold change >100).

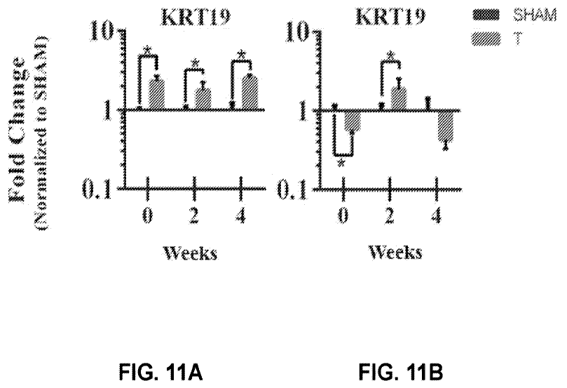

Expression of NP marker KRT19 was significantly increased in BrachT transfected PD cells at 2 weeks compared to SHAM controls while ND cells showed significant increases at all time-points (FIGS. 11A and 11B, p<0.05).

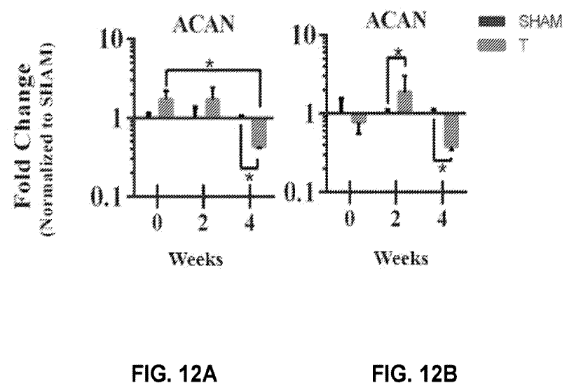

Expression of matrix protein ACAN was increased at 2 weeks in transfected ND and PD cells (significant for PD cells) with significant decreases at 4 weeks for both groups (FIGS. 12A and 12B).

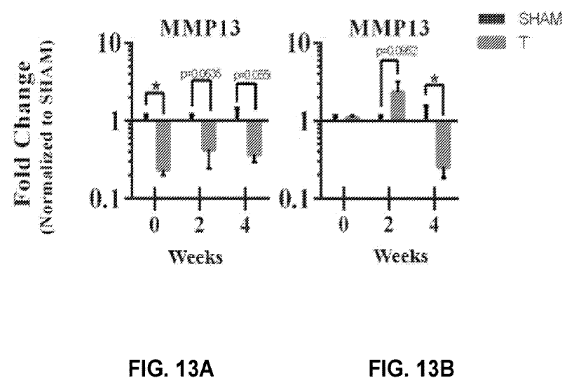

Expression of MMP13 was significantly decreased in transfected ND cells at all time points and demonstrated a significant decrease at week 4 for transfected PD cells (FIGS. 13A and 13B).

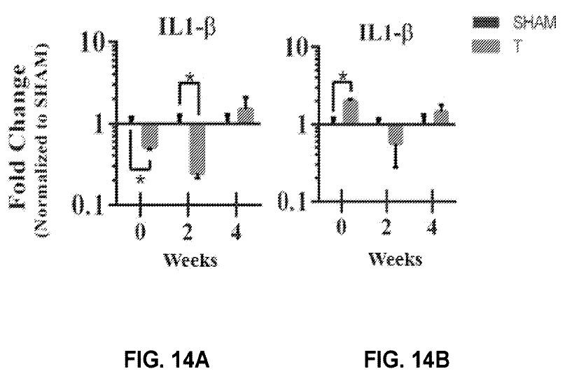

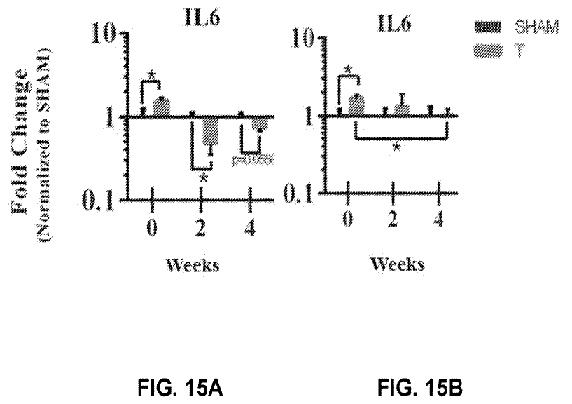

Pro-inflammatory cytokines IL-13 (FIGS. 14A and 14B) and IL-6 (FIGS. 15A and 15B) demonstrated decreased expression at 2 weeks for transfected ND samples but showed an initial increase for transfected PD samples that decreased with time.

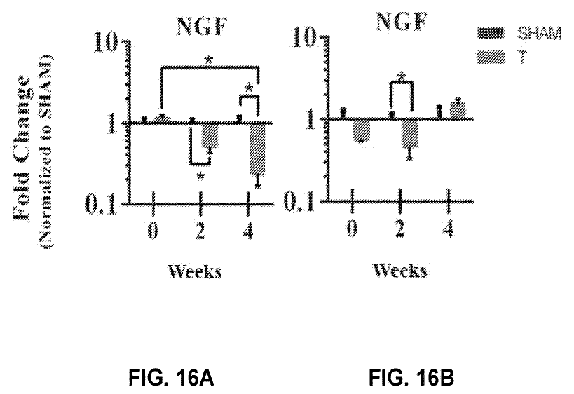

Nerve growth factor (NGF) showed a significant decrease in expression at week 2 for transfected PD cells but significant decreases at 2 and 4 weeks in transfected ND cells (FIGS. 16A and 16B).

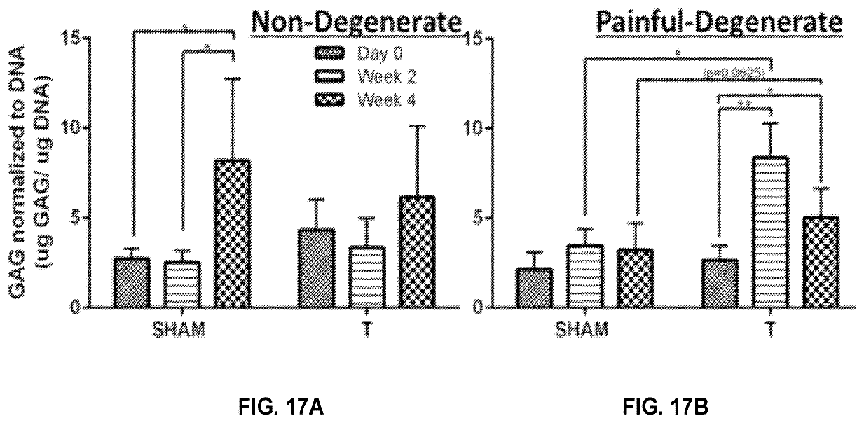

PD cells demonstrated a significant increase in GAG content in BrachT transfected groups at 2 weeks compared to their respective SHAM and this was observed to a lesser extent at week 4 (FIGS. 17A and 17B). Autopsy samples demonstrated an increase in GAG at 4 weeks in SHAM groups with no significant differences in BrachT transfected groups.

### DISCUSSION

These results demonstrate that human NP cells can be successfully transfected with transcription factor BrachT and reprogrammed to a healthy NP phenotype with up-regulation of key phenotypic markers, enhanced proteoglycan synthesis and down-regulation of inflammatory, catabolic and pain-related markers. High expression of BrachT was maintained over 4 weeks in 3D culture without any detrimental effects on cell viability. ND cells transfected with BrachT demonstrated increases in gene expression for healthy NP marker KRT19, decreases in MMP13 suggesting a decrease in catabolism, decreases in pro-inflammatory and pain genes IL-1p, IL-6, and NGF which all suggest reprogramming towards a ‘healthier’ IVD phenotype. While similar effects were observed in PD cells this was considered more temporal with peak anabolic effects observed at 2 weeks. Temporal effects suggest further optimization of the delivery system as bulk electroporation involves disruption of the cellular membrane and is less efficient compared to techniques such as engineered vesicles or tissue nanotransfection [Gallego-Perez et al, 2017]. The same temporal effects are seen at week 2 in GAG content with significant increases in GAG compared to the SHAM group at 2 weeks only. In conclusion, this study demonstrated the potential of BrachT to promote a healthy IVD phenotype via transfection into human ND and PD NP cells with increased GAG accumulation.

This is the first study to demonstrate successful reprogramming of diseased human NP cells into healthy NP cells using non-viral transfection of transcription factor BrachT. Further development of this treatment in conjunction with novel, minimally invasive tissue nanotransfection methods has high potential as a regenerative strategy for the treatment of LBP and other musculoskeletal diseases.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### Example 3: Extracellular Vesicle Delivery of Transcription Factors to Reprogram Cells In-Vitro

### Methods:

FOXF1 plasmids were expanded and transfected into NP as in cells with culture media collected at 48 hours and extracellular vesicles isolated (Total exosome Isolation Kit). Exosomes were introduced to separate NP cells in monolayer with FOXF1 gene expression assessed at 2 and 7 days. Non-parametric statistical tests were used (Mann-Whitney Tests, α=0.05).

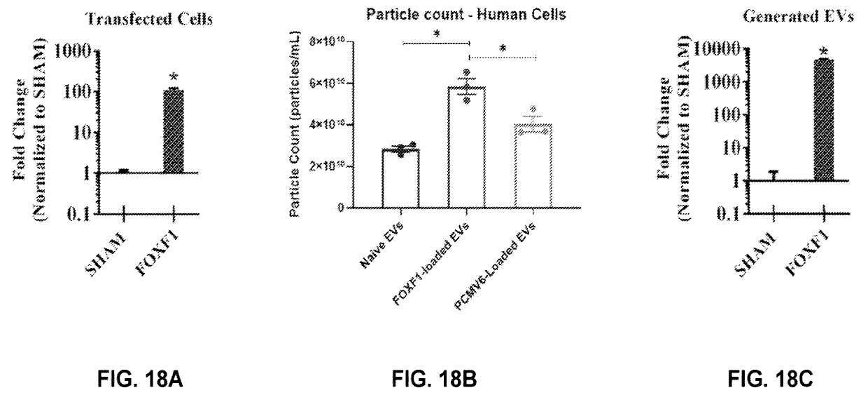

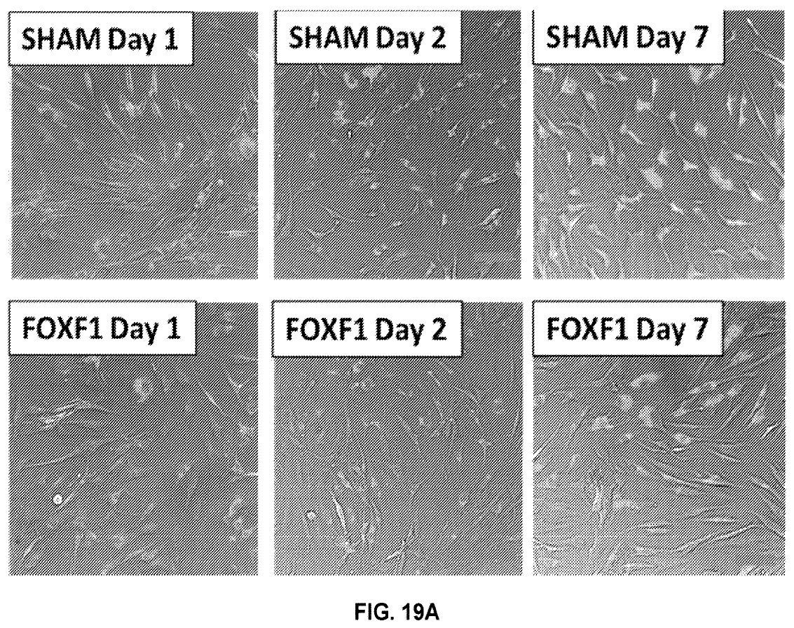

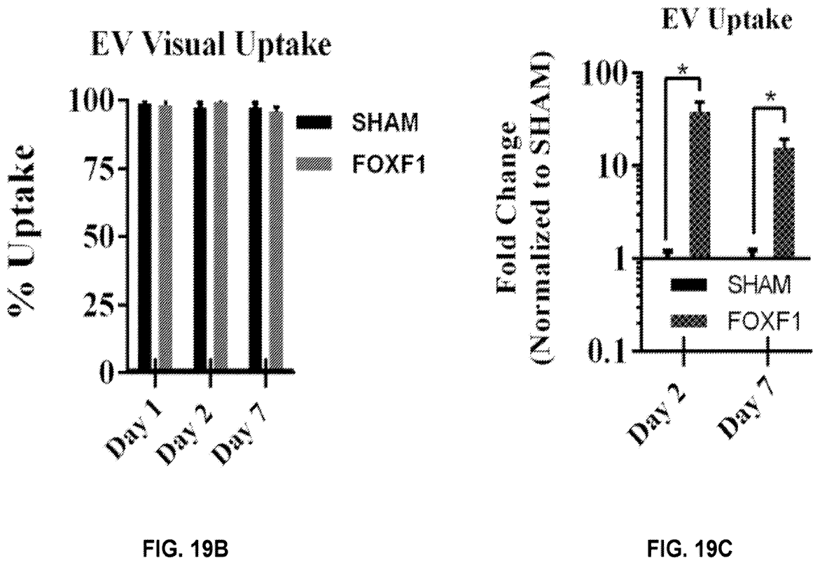

Results: When human EVs labelled with membrane dye PKH26 (red) were incubated with human NP cells in-vitro, uptake of labelled EVs was observed for SHAM and FOXF1 groups (FIG. 19A). In addition, >1000 fold increases in FOXF1 gene expression in FOXF1-EVs compared to SHAM-EVs was also observed. When human NP cells were treated with FOXF1-EVs they demonstrated significant upregulation of FOXF1 at both 2 and 7 days in culture compared to SHAM-EV controls. (FIG. 18B,C) Conclusion:

Tagged EVs showed high efficiency in microscopic images and high expression of FOXF1 gene packaged within EVs. Significant upregulation of FOXF1 in FOXF1 EV treated cells implies successful transfection of NP cells using generated EVs. This study demonstrated the potential of FOXF1 to promote a healthy IVD phenotype via transfection into degenerate human NP cells using EVs as a delivery mechanism.

### Example 4: Extracellular Vesicle Delivery of Transcription Factors to Reprogram Cells In-Vivo

Method: EVs were generated as before and injected into mice lumbar intervertebral disc in-vivo and accessed for 7 days (N=3). In an ongoing study, mice discs were punctured and will be accessed biweekly over 12 and 24 weeks.

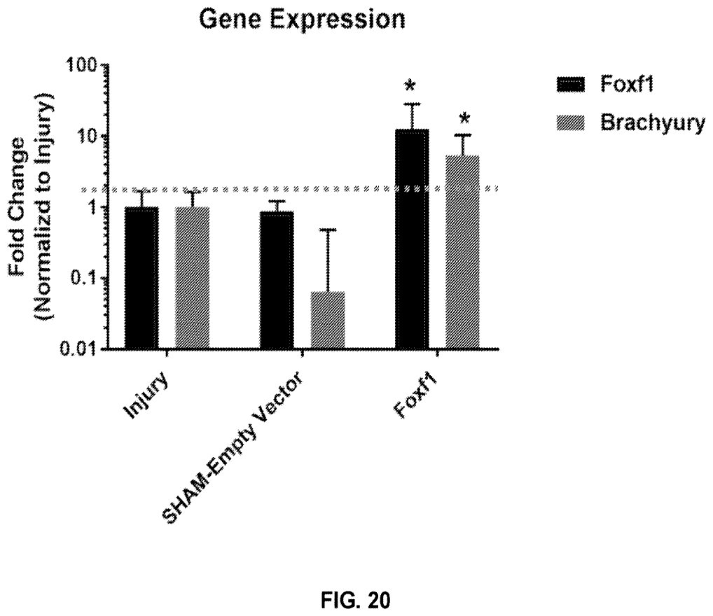

Results: Viability staining of Mouse disc showed no cytotoxicity compared to non-injected control discs and injected discs showed upregulation of FOXF1 along with healthy NP marker brachyury (FIG. 20)

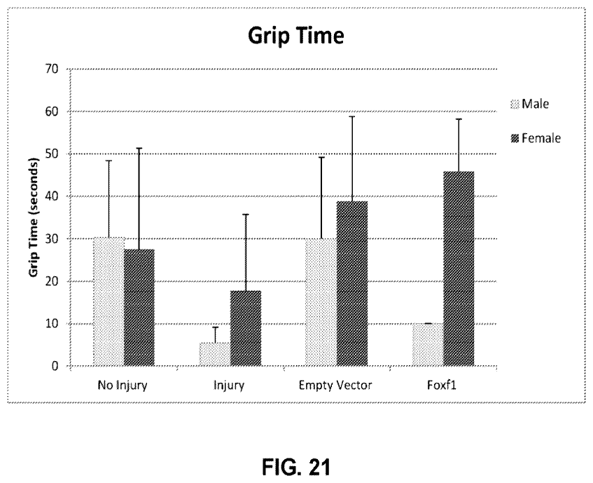

Conclusion: This experiment shows the non-cytotoxic effects of transcription factor delivery via EVs and that the transcription factor successfully integrates into the intervertebral disc space along with upregulation of a healthy marker that was not Injected into the disc. Furthermore, ongoing studies show behavioral differences between injured untreated mice compared to foxf1 treated mice as seen in FIG. 21 where treated mice exhibit longer grip time (indicative of axial strength) compared to injured groups.

What is claimed is:

1. A method for treating a musculoskeletal disease in a subject, comprising
(a) non-virally delivering intracellularly into musculoskeletal cells of the subject one or more transcription factor proteins selected from the group comprising HIF-1α, HIF-2α, T-box family protein, a Forkhead-box (FOX) family protein, a Mohawk family protein, Scleraxis, NFAT Family protein, C-1-1, PGC1α, Osterix, MEF2C, Sonic hedgehog pathway protein, PAX family protein, SOX family protein, notochord homeobox, Tenomodulin, Nkx3-2, RUNX2, API family proteins, Afp36, Ebf family proteins, MAF, NUPR1, Twist family protein, MAGED1, SATB2, LMP3, Oct family protein, Dlx family proteins, C/ECPs, ATF family proteins, SMADs, MEN family proteins, MSX family proteins, NF-1, SP3, Ob1, EGR family proteins, SIX family proteins, EYA family prteisn, PEA3, BCL6, Myo family Proteins, CSFR pathway proteins, SRG, GLI2, Sp4, ATF family proteins, ETV family proteins, and REST or polynucleotides encoding the one or more transcription factor proteins;
(b) exposing the musculoskeletal cells to an extracellular vesicle produced from a cell containing or expressing the one or more transcription factor proteins, or polynucleotides encoding the one or more transcription factor proteins.

2. The method of claim 1, wherein the musculoskeletal cells comprises diseased nucleus pulposus (NP) cells, and wherein the one or more transcription factor proteins comprises HIF-1α, HIF-2α, a T-box family protein, a Sonic Hedgehod signalong pathway, PAX1, SOX family protein, NOTO, a Forkhead-box (FOX) family protein, or any combination thereof.

3. The method of claim 1, wherein the musculoskeletal cells comprises annulus fibrosis (AF) cells, and wherein the one or more transcription factor proteins comprises a Mohawk family protein, Tenomodulin, PAX9, Scleraxis, or any combination thereof.

4. The method of claim 1, wherein the musculoskeletal cells comprises cartilage endplate cells, and wherein the one or more transcription factor proteins comprises NFAT Family protein, C-1-1, PGC1α, Osterix, SOX family protein, Nkx3-2, MEF2C, or any combination thereof.

5. The method of claim 1, wherein the musculoskeletal cells comprises Osteocytes, Osteoclasts, Osteoblasts, and wherein the one or more transcription factor proteins comprises RUNX2, Fox family proteins, API complex proteins, Afp36, Ebf famil proteins, Naf, Nef2c, Twist family proteins, MAGED1, SATB2, LMP3, OCT family proteins, KLF4, MYC, DLX family protens, C/EBPs, ATF family proteins, NFATc, SMADS, Menin, Msx family proteins, SP3, Ob-1, NF-1, or any combination thereof.

6. The method of claim 1, wherein the musculoskeletal cells comprises Tenocytes and Ligament cells, and wherein the one or more transcription factor proteins comprises EGR family proteins, Scleraxis, NKx, SIX family proteins, EYA family proteins, PEA3 and Mohawk.

7. The method of claim 1, wherein the musculoskeletal cells comprises Synoviocytes, and wherein the one or more transcription factor proteins comprises SOX family proteins, NFAT5, BCL-6, HIF-1α, and HIF-2α.

8. The method of claim 1, wherein the musculoskeletal cells comprises Monocytes and Myofibriblasts, and wherein the one or more transcription factor proteins comprises Myo family proteins, PU1, CSFR Pathway proteins, EBPA, SRF, GLI2.

9. The method of claim 1, wherein the musculoskeletal cells comprises Dorsal Root Ganglion Cells, and wherein the one or more transcription factor proteins comprises FOXO, SP4, ATF family proteins, ETV family proteins, SOX11, REST, RUNX1, and RUNX3.

10. A non-viral vector comprising a polynucleotide comprising a nucleic acid sequence encoding two or more transcription factors selected from the group comprising Forkhead-box (FOX) family protein, a Mohawk family protein, Scleraxis, NFAT Family protein, C-1-1, PGC1α, Osterix, and MEF2C, and factors from Table 1 operably linked to an expression control sequence, wherein the non-viral vector is encapsulated in a liposome, microparticle, or nanoparticle suitable for intracellular delivery.
